(19) United States
(12) Patent Application Publication
(10) Pub. No.: US 2024/0107876 A1
(43) Pub. Date: Mar. 28, 2024

(54) ORGANIC ELECTROLUMINESCENCE DEVICE

(71) Applicant: IDEMITSU KOSAN CO.,LTD., Tokyo (JP)

(72) Inventors: Hiroaki TOYOSHIMA, Sodegaura-shi (JP); Masatoshi SAITO, Sodegaura-shi (JP); Kei YOSHIDA, Sodegaura-shi (JP); Masato MITANI, Sodegaura-shi (JP); Sayaka MIZUTANI, Sodegaura-shi (JP)

(73) Assignee: IDEMITSU KOSAN CO.,LTD., Tokyo (JP)

(21) Appl. No.: 18/269,482

(22) PCT Filed: Dec. 6, 2021
(86) PCT No.: PCT/JP2021/044702

§ 371 Date: Jun. 23, 2023

(30) Foreign Application Priority Data
Dec. 25, 2020 (JP) 2020-217143

(51) Int. Cl. H10K 85/60; C07D 401/10; C07D 401/14; C07D 409/14; C09K 11/06

(52) U.S. Cl. CPC H10K 85/615; C07D 401/10; C07D 401/14; C07D 409/14; C09K 11/06; H10K 85/622; H10K 85/654; H10K 85/6572; H10K 85/6576; H10K 50/16

(57) ABSTRACT

An organic electroluminescence device including a cathode, an anode, an emitting layer arranged between the cathode and the anode, and an electron-transporting region arranged between the emitting layer and the cathode, wherein the electron-transporting region includes one or more compounds selected from the group consisting of compounds represented by the following formulas (1) to (4) and a rare earth element, and the electron-transporting region substantially does not include an alkali metal, a compound containing an alkali metal, a metal belonging to Group 13 of the Periodic Table of the Elements, and a compound having a metal belonging to Group 13 of the Periodic Table of the Elements.

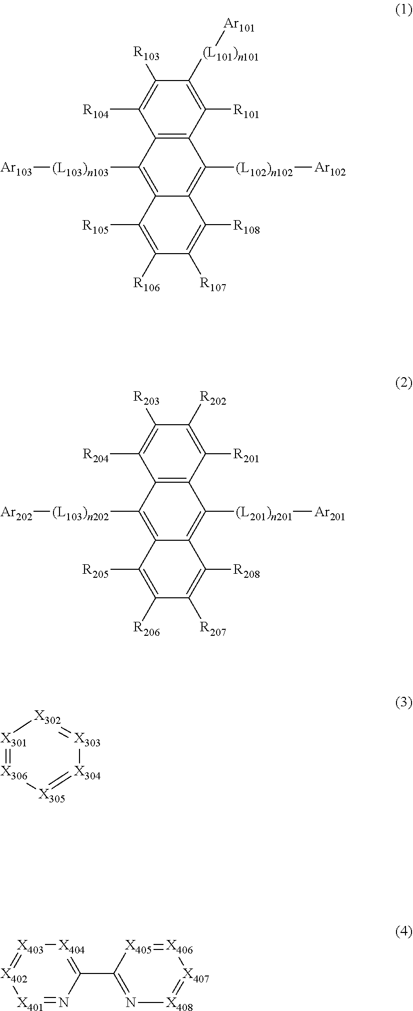

### TECHNICAL FIELD

The present invention relates to an organic electroluminescence device and an electronic apparatus.

### BACKGROUND ART

When voltage is applied to an organic electroluminescence device (hereinafter, also referred to as an organic EL device), holes and electrons are injected into an emitting layer from an anode and a cathode, respectively. Then, thus injected holes and electrons are recombined in the emitting layer, and excitons are formed therein.

In conventional organic EL devices, a compound having high electron-injecting property such as 8-hydroxyquinolinolato-lithium (hereinafter, also referred to as a “Liq”), tris (8-quinolinolato) aluminum (hereinafter, also referred to as an “Alq3”) and bis (2-methyl-8-quinolinolato)-4-(phenylphenolato) aluminum (hereinafter, also referred to as a “BAlq”) is used in combination with other electron-transporting material in an electron-transporting region arranged between the cathode and the emitting layer for ensuring electron-injecting property (for example, see Patent Document 1).

### RELATED ART DOCUMENTS

### Patent Documents

[Patent Document 1] WO 2016/084962 A1

### SUMMARY OF THE INVENTION

When a material such as Liq, Alq3 and BAlq is used for the organic EL device, the device performance thereof is basically increased. However, there was a problem that at least a part of the device performance thereof is decreased, and which was caused by a difference of the electron mobility thereof from that of other material used in the electron-transporting region, and the like. Further, a mixed layer of these materials and electron-transporting materials is formed by co-depositing several materials, but there was a problem that fabricating processes were complicated.

It is an object of the present invention to provide an organic EL device capable of exhibiting high device performance without substantially using a material having high electron-injecting property such as Liq, Alq3 and Balq in the electron-transporting region.

As a result of extensive studies, the present inventors have found that when the specific material is used as a material constituting the electron-transporting region, sufficient electron-transporting property can be ensured without using a material having high electron-injecting property such as Liq, Alq3 and Balq, and further, disadvantages caused by Liq, Alq3, Balq, and the like are overcome to be capable of greatly improving the device performance in some cases as compared to the case where these materials are used, and has completed the present invention.

According to the present invention, the following organic EL device and the like are provided.

An organic electroluminescence device comprising
a cathode,
an anode,
an emitting layer arranged between the cathode and the anode, and
an electron-transporting region arranged between the emitting layer and the cathode,
wherein the electron-transporting region comprises one or more compounds selected from the group consisting of compounds represented by the following formulas (1) to (4) and a rare earth element, and
the electron-transporting region substantially does not comprise an alkali metal, a compound containing an alkali metal, a metal belonging to Group 13 of the Periodic Table of the Elements, and a compound having a metal belonging to Group 13 of the Periodic Table of the Elements:

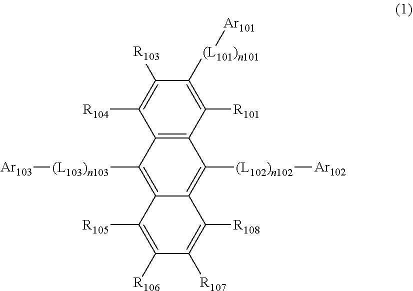

wherein in the formula (1),
R₁₀₁ and R₁₀₃ to R₁₀₈ are independently a hydrogen atom, or a substituent R;
L₁₀₁ is
a single bond,
a substituted or unsubstituted arylene group having 6 to 50 ring carbon atoms, or
a substituted or unsubstituted divalent heterocyclic group having 5 to 50 ring atoms;
L₁₀₂ and L₁₀₃ are independently
a single bond, or
a substituted or unsubstituted arylene group having 6 to 50 ring carbon atoms;
Ar₁₀₁ is
a substituted or unsubstituted monovalent group including a nitrogen-containing six-membered ring, or
a substituted or unsubstituted benzimidazolyl group;
Ar₁₀₂ and Ar₁₀₃ are independently a substituted or unsubstituted aryl group having 6 to 50 ring carbon atoms;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other), a halogen atom, a cyano group, a nitro group,
a substituted or unsubstituted aryl group having 6 to 50 ring carbon atoms, and
a substituted or unsubstituted monovalent heterocyclic group having 5 to 50 ring atoms; when two or more substituents R are present, the two or more substituents R may be the same as or different from each other;
n101 is an integer of 1 to 3; when n101 is 2 or more, two or more L₁₀₁'s may be the same as or different from each other;
n102 is an integer of 1 to 3; when n102 is 2 or more, two or more L₁₀₂'s may be the same as or different from each other;
n103 is an integer of 1 to 3; when n103 is 2 or more, two or more L₁₀₃'s may be the same as or different from each other:

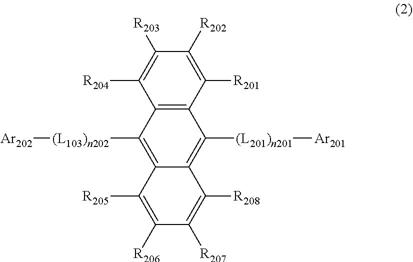

wherein in the formula (2),
R₂₀₁ to R₂₀₈ are independently a hydrogen atom, or a substituent R;
L₂₀₁ and L₂₀₂ are independently
a single bond, or
a substituted or unsubstituted arylene group having 6 to 50 ring carbon atoms;
Ar₂₀₁ and Ar₂₀₂ are independently a substituted or unsubstituted monovalent heterocyclic group having 5 to 50 ring atoms;
n201 is an integer of 1 to 3; when n201 is 2 or more, two or more L₂₀₁'s may be the same as or different from each other;
n202 is an integer of 1 to 3; when n202 is 2 or more, two or more L₂₀₂'s may be the same as or different from each other;
the substituent R is the same as defined in the formula (1):

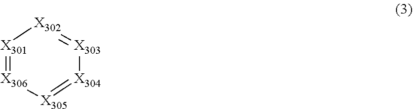

wherein in the formula (3),
X₃₀₁ to X₃₀₆ are independently N, CR₃₀₁ or CR₃₀₂; provided that at least two of X₃₀₁ to X₃₀₆ are N, at least one thereof is CR₃₀₁, and at least two thereof are CR₃₀₂; when two or more R₃₀₁'s are present, the two or more R₃₀₁'s may be the same as or different from each other; two or more R₃₀₂'s may be the same as or different from each other;
R₃₀₁ is a monovalent group represented by the following formula (3A):

(Ar₃₀₁)^{m3A}-(L₃₀₁)_{n3A}-* (3A)

wherein in the formula (3A),
L₃₀₁ is
a single bond,
a substituted or unsubstituted aromatic hydrocarbon ring group having 6 to 50 ring carbon atoms, or
a substituted or unsubstituted heterocyclic group having 5 to 50 ring atoms;
Ar₃₀₁ is
a substituted or unsubstituted dibenzofuranyl group,
a substituted or unsubstituted dibenzothiophenyl group,
a substituted or unsubstituted carbazolyl group,
a substituted or unsubstituted phenanthryl group,
a substituted or unsubstituted monovalent group including a nitrogen-containing five-membered ring, or
a monovalent group represented by the following formula (3B):

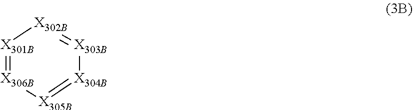

wherein in the formula (3B),
X_{301B} to X_{306B} are independently N, CR_{301B} or CR_{302B}; provided that one of X_{301B} to X_{306B} is CR_{301B}, and at least one thereof is N;
R_{301B} is a single bond bonding with L₃₀₁;
when two or more R_{302B}'S are present, the adjacent two of R_{302B}'S form a substituted or unsubstituted, saturated or unsaturated fused ring by bonding with each other, or do not form the substituted or unsubstituted, saturated or unsaturated fused ring;
R_{302B} which do not form the substituted or unsubstituted, saturated or unsaturated fused ring is a hydrogen atom, or a substituent R;
when two or more R_{302B}'S are present, the two or more R_{302B}'s may be the same as or different from each other;
m3A is an integer of 1 to 5; when m3A is 2 or more, two or more Ar₃₀₁'s may be the same as or different from each other;
n3A is an integer of 1 to 3; when n3A is 2 or more, two or more L₃₀₁'s may be the same as or different from each other; provided that when all of L₃₀₁'s are a single bond, m3A is 1;
R₃₀₂ is a hydrogen atom, or a substituent R;
the substituent R is the same as defined in the formula (1):

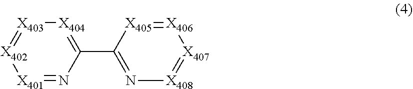

wherein in the formula (4),
X₄₀₁ to X₄₀ₛ are independently N, CR₄₀₁ or CR₄₀₂; provided that at least one of X₄₀₁ to X₄₀₈ is CR₄₀₁; when two or more R₄₀₁'s are present, the two or more R₄₀₁'s may be the same as or different from each other; when two or more R₄₀₂'s are present, the two or more R₄₀₂'s may be the same as or different from each other;
when both of X₄₀₄ and X₄₀₅ are CR₄₀₂, the two R₄₀₂'s form a substituted or unsubstituted, saturated or unsaturated fused ring by bonding with each other, or do not form the substituted or unsubstituted, saturated or unsaturated fused ring;
R₄₀₁ is a monovalent group represented by the following formula (4A):

(Ar₄₀₁)_{m4A}-(L₄₀₁)_{n4A}—* (4A)

wherein in the formula (4A),
L₄₀₁ is
a single bond,
a substituted or unsubstituted aromatic hydrocarbon ring group having 6 to 50 ring carbon atoms, or
a substituted or unsubstituted heterocyclic group having 5 to 50 ring atoms;
Ar₄₀₁ is
a hydrogen atom,
a substituted or unsubstituted aryl group having 6 to 50 ring carbon atoms, or
a substituted or unsubstituted monovalent heterocyclic group having 5 to 50 ring atoms; m4A is an integer of 1 to 5; when m4A is 2 or more, two or more Ar₄₀₁'s may be the same as or different from each other;
n4A is an integer of 1 to 3; when n4A is 2 or more, two or more L₄₀₁'s may be the same as or different from each other; provided that when all of L₄₀₁'s are a single bond, m4A is 1;
R₄₀₂ which do not form the substituted or unsubstituted, saturated or unsaturated fused ring is a hydrogen atom, or a substituent R; and the substituent R is the same as defined in the formula (1).

According to the present invention, there can be provided an organic EL device capable of exhibiting high device performance without substantially using a material having high electron-injecting property such as Liq, Alq3 and Balq in the electron-transporting region.

### BRIEF DESCRIPTION OF THE DRAWINGS

FIG. 1 is a diagram showing a schematic configuration of an organic EL device according to an aspect of the present invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group“is a” ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as described in this specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

“Substituted or unsubstituted aryl group” Specific examples of the “substituted or unsubstituted aryl group” described in this specification (specific example group G1) include the following unsubstituted aryl groups (specific example group G1A), substituted aryl groups (specific example group G11B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”.). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.
Unsubstituted aryl group (specific example group G1A):
   a phenyl group,
   a p-biphenyl group,
   a m-biphenyl group,
   an o-biphenyl group,
   a p-terphenyl-4-yl group,
   a p-terphenyl-3-yl group,
   a p-terphenyl-2-yl group,
   a m-terphenyl-4-yl group,
   a m-terphenyl-3-yl group,
   a m-terphenyl-2-yl group,
   an o-terphenyl-4-yl group,
   an o-terphenyl-3-yl group,
   an o-terphenyl-2-yl group,
   a 1-naphthyl group,
   a 2-naphthyl group,
   an anthryl group,
   a benzanthryl group,
   a phenanthryl group,
   a benzophenanthryl group,
   a phenalenyl group,
   a pyrenyl group,
   a chrysenyl group,
   a benzochrysenyl group,
   a triphenylenyl group,
   a benzotriphenylenyl group,
   a tetracenyl group,
   a pentacenyl group,
   a fluorenyl group,
   a 9,9′-spirobifluorenyl group,
   a benzofluorenyl group,
   a dibenzofluorenyl group,
   a fluoranthenyl group,
   a benzofluoranthenyl group,
   a perylenyl group, and
   a monovalent aryl group derived by removing one hydrogen atom from the ring structures represented by any of the following general formulas (TEMP-1) to (TEMP-15).

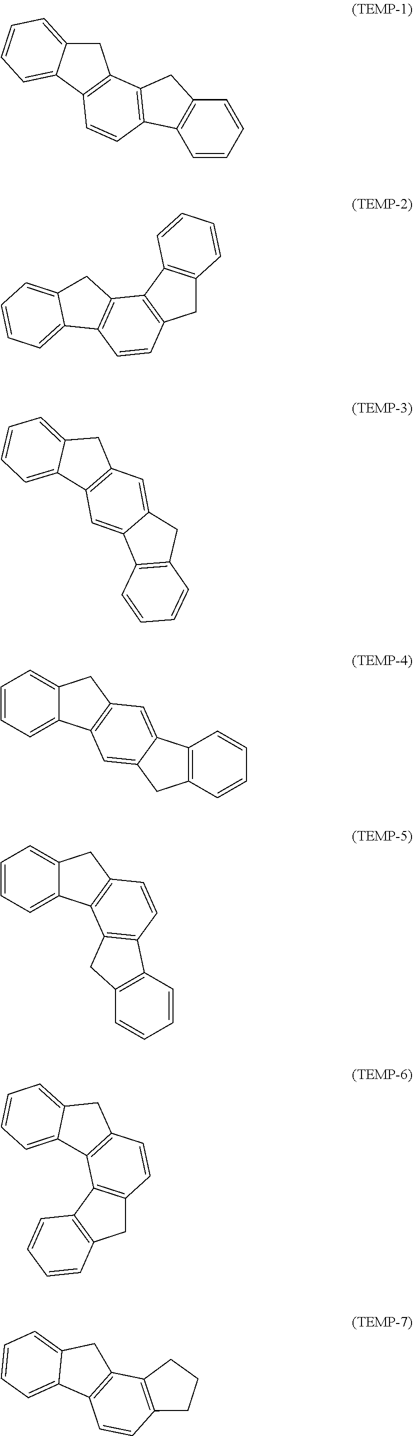

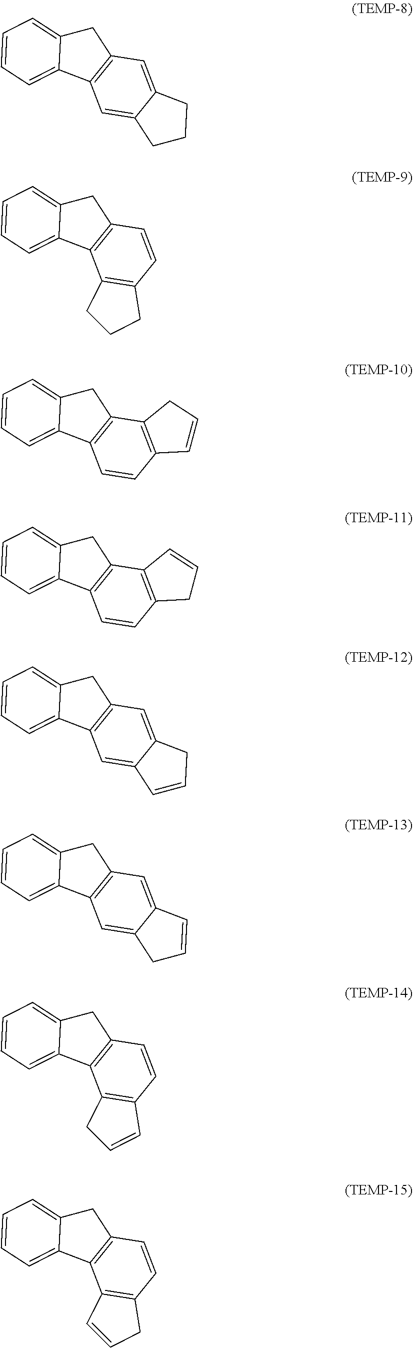

Substituted aryl group (specific example group G1B):
   an o-tolyl group,
   a m-tolyl group,
   a p-tolyl group,
   a p-xylyl group,
   a m-xylyl group,
   an o-xylyl group,
   a p-isopropylphenyl group,
   a m-isopropylphenyl group,
   an o-isopropylphenyl group,
   a p-t-butylphenyl group,
   a m-t-butylphenyl group,
   an o-t-butylphenyl group,
   a 3,4,5-trimethylphenyl group,
   a 9,9-dimethylfluorenyl group,
   a 9,9-diphenylfluorenyl group,
   a 9,9-bis(4-methylphenyl)fluorenyl group,
   a 9,9-bis(4-isopropylphenyl)fluorenyl group,
   a 9,9-bis(4-t-butylphenyl)fluorenyl group,
   a cyanophenyl group,
   a triphenylsilylphenyl group,
   a trimethylsilylphenyl group,
   a phenylnaphthyl group,
   a naphthylphenyl group, and
   a group in which one or more hydrogen atoms of a monovalent group derived from the ring structures represented by any of the general formulas (TEMP-1) to (TEMP-15) are substituted by a substituent.
“Substituted or unsubstituted heterocyclic group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group“is a” heterocyclic group substituted by a substituent”.). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B 1), the following substituted heterocyclic group containing an oxygen atom (specific example group G2B 2), the following substituted heterocyclic group containing a sulfur atom (specific example group G2B 3), and the following group in which one or more hydrogen atoms of the monovalent heterocyclic group derived from the ring structures represented by any of the following general formulas (TEMP-16) to (TEMP-33) are substituted by a substituent (specific example group G2B 4).
Unsubstituted heterocyclic group containing a nitrogen atom (specific example group G2A1):
   a pyrrolyl group,
   an imidazolyl group,
   a pyrazolyl group,
   a triazolyl group,
   a tetrazolyl group,
   an oxazolyl group,
   an isoxazolyl group,
   an oxadiazolyl group,
   a thiazolyl group,
   an isothiazolyl group,
   a thiadiazolyl group,
   a pyridyl group,
   a pyridazinyl group,
   a pyrimidinyl group,
   a pyrazinyl group,
   a triazinyl group,
   an indolyl group,
   an isoindolyl group,
   an indolizinyl group,
   a quinolizinyl group,
   a quinolyl group,
   an isoquinolyl group,
   a cinnolyl group,
   a phthalazinyl group,
   a quinazolinyl group,
   a quinoxalinyl group,
   a benzimidazolyl group,
   an indazolyl group,
   a phenanthrolinyl group,
   a phenanthridinyl group,
   an acridinyl group,
   a phenazinyl group,
   a carbazolyl group,
   a benzocarbazolyl group,
   a morpholino group,
   a phenoxazinyl group,
   a phenothiazinyl group,
   an azacarbazolyl group, and
   a diazacarbazolyl group.
Unsubstituted heterocyclic group containing an oxygen atom (specific example group G2A2):
   a furyl group,
   an oxazolyl group,
   an isoxazolyl group,
   an oxadiazolyl group,
   a xanthenyl group,
   a benzofuranyl group,
   an isobenzofuranyl group,
   a dibenzofuranyl group,
   a naphthobenzofuranyl group,
   a benzoxazolyl group,
   a benzisoxazolyl group,
   a phenoxazinyl group,
   a morpholino group,
   a dinaphthofuranyl group,
   an azadibenzofuranyl group,
   a diazadibenzofuranyl group,
   an azanaphthobenzofuranyl group, and
   a diazanaphthobenzofuranyl group.
Unsubstituted heterocyclic group containing a sulfur atom (specific example group G2A3):
   a thienyl group,
   a thiazolyl group,
   an isothiazolyl group,
   a thiadiazolyl group,
   a benzothiophenyl group (benzothienyl group),
   an isobenzothiophenyl group (isobenzothienyl group),
   a dibenzothiophenyl group (dibenzothienyl group),
   a naphthobenzothiophenyl group (naphthobenzothienyl group),
   a benzothiazolyl group,
   a benzisothiazolyl group,
   a phenothiazinyl group,
   a dinaphthothiophenyl group (dinaphthothienyl group),
   an azadibenzothiophenyl group (azadibenzothienyl group),
   a diazadibenzothiophenyl group (diazadibenzothienyl group),
   an azanaphthobenzothiophenyl group (azanaphthobenzothienyl group), and
   a diazanaphthobenzothiophenyl group (diazanaphthobenzothienyl group).
Monovalent heterocyclic group derived by removing one hydrogen atom from the ring structures represented by any of the following general formulas (TEMP-16) to (TEMP-33) (specific example group G2A4):

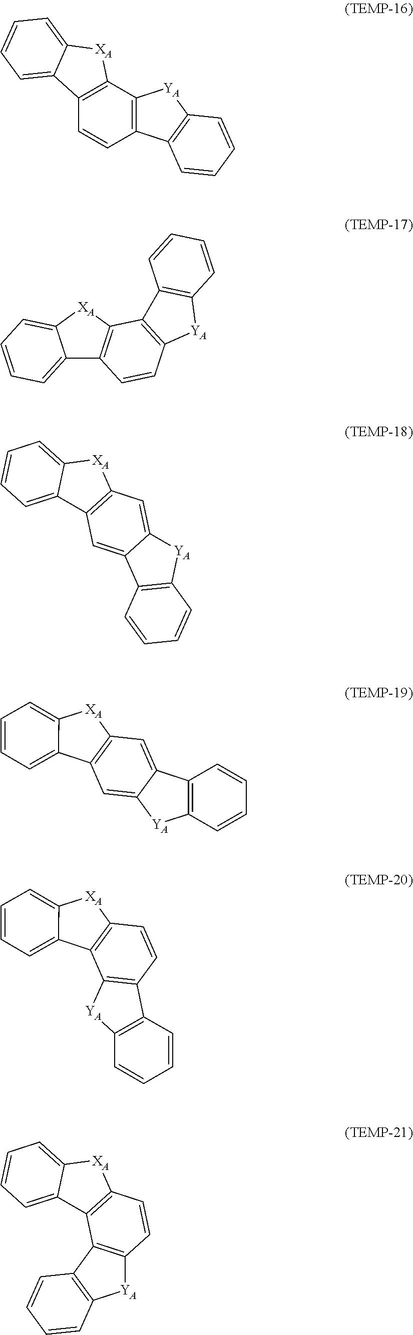

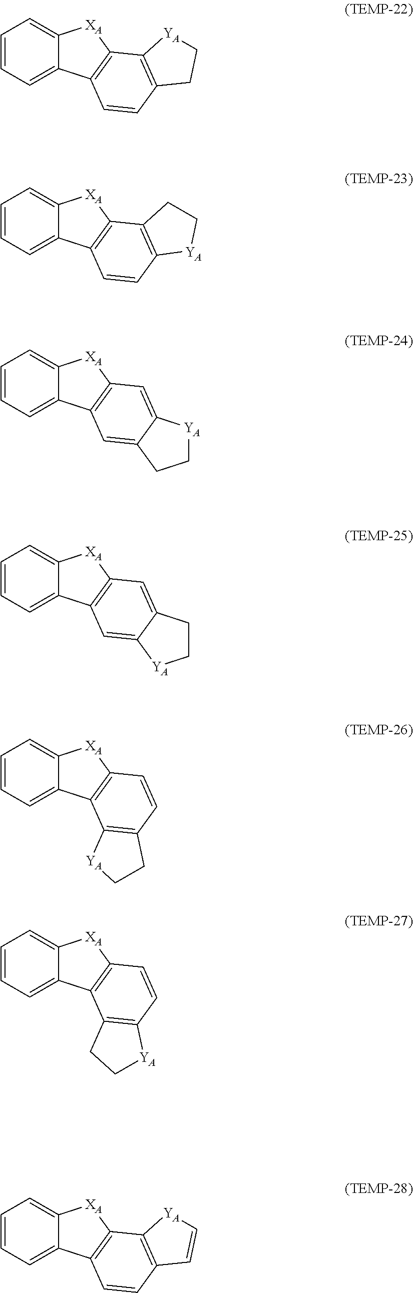

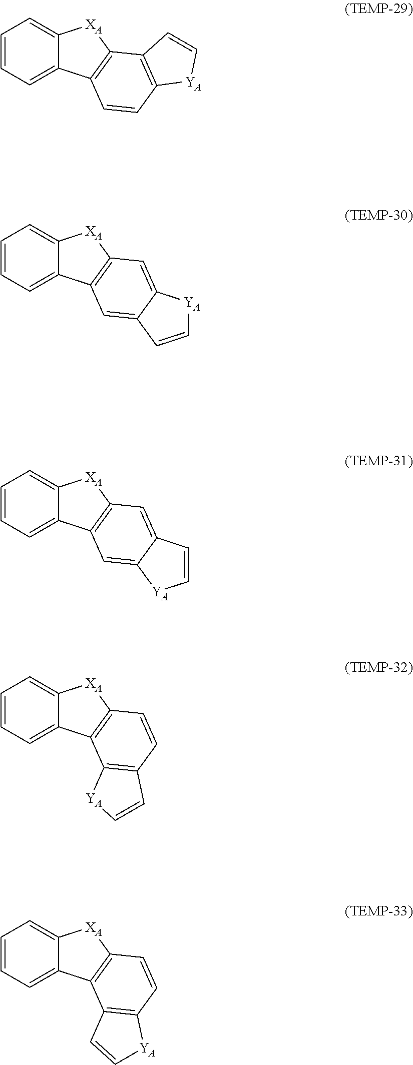

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.
Substituted heterocyclic group containing a nitrogen atom (specific example group G2B 1):
   a (9-phenyl)carbazolyl group,
   a (9-biphenylyl)carbazolyl group,
   a (9-phenyl)phenylcarbazolyl group,
   a (9-naphthyl)carbazolyl group,
   a diphenylcarbazol-9-yl group,
   a phenylcarbazol-9-yl group,
   a methylbenzimidazolyl group,
   an ethylbenzimidazolyl group,
   a phenyltriazinyl group,
   a biphenylyltriazinyl group,
   a diphenyltriazinyl group,
   a phenylquinazolinyl group, and
   a biphenylylquinazolinyl group.
Substituted heterocyclic group containing an oxygen atom (specific example group G2B 2):
   a phenyldibenzofuranyl group,
   a methyldibenzofuranyl group,
   a t-butyldibenzofuranyl group, and
   a monovalent residue of spiro[9H-xanthene-9,9′-[9H]fluorene].
Substituted heterocyclic group containing a sulfur atom (specific example group G2B 3):
   a phenyldibenzothiophenyl group,
   a methyldibenzothiophenyl group,
   a t-butyldibenzothiophenyl group, and
   a monovalent residue of spiro[9H-thioxanthene-9,9′-[9H]fluorene].
Group in which one or more hydrogen atoms of the monovalent heterocyclic group derived from the ring structures represented by any of the following general formulas (TEMP-16) to (TEMP-33) are substituted by a substituent (specific example group G2B 4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.
“Substituted or unsubstituted alkyl group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”.). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.
Unsubstituted alkyl group (specific example group G3A):
   a methyl group,
   an ethyl group,
   a n-propyl group,
   an isopropyl group,
   a n-butyl group,
   an isobutyl group,
   a s-butyl group, and
   a t-butyl group.
Substituted alkyl group (specific example group G3B):
   a heptafluoropropyl group (including isomers),
   a pentafluoroethyl group,
   a 2,2,2-trifluoroethyl group, and
   a trifluoromethyl group.
“Substituted or unsubstituted alkenyl group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group“is a” alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.
Unsubstituted alkenyl group (specific example group G4A):
   a vinyl group,
   an allyl group,
   a 1-butenyl group,
   a 2-butenyl group, and
   a 3-butenyl group.
Substituted alkenyl group (specific example group G4B):
   a 1,3-butanedienyl group,
   a 1-methylvinyl group,
   a 1-methylallyl group,
   a 1,1-dimethylallyl group,
   a 2-methylally group, and
   a 1,2-dimethylallyl group.
“Substituted or unsubstituted alkynyl group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”.). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.
Unsubstituted alkynyl group (specific example group G5A):
   an ethynyl group.
“Substituted or unsubstituted cycloalkyl group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group“is a” cycloalkyl group substituted by a substituent”.). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.
Unsubstituted cycloalkyl group (specific example group G6A):
   a cyclopropyl group,
   a cyclobutyl group,
   a cyclopentyl group,
   a cyclohexyl group,
   a 1-adamantyl group,
   a 2-adamantyl group,
   a 1-norbornyl group, and
   a 2-norbornyl group.
Substituted cycloalkyl group (specific example group G6B):
   a 4-methylcyclohexyl group.
“Group represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.
“Group represented by —O—(R₉₀₄)”

Specific examples of the group represented by —O—(R₉₀₄) in this specification (specific example group G8) include:
—O(G1),
—O(G2),
—O(G3), and
—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.
“Group represented by —S—(R₉₀₅)”

Specific examples of the group represented by —S—(R₉₀₅) in this specification (specific example group G9) include:
—S(G1),
—S(G2),
—S(G3), and
—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group represented by —N(R₉₀₆)(R₉₀₇)”

Specific examples of the group represented by —N(R₉₀₆)(R₉₀₇) in this specification (specific example group G10) include:
—N(G1)(G1),
—N(G2)(G2),
—N(G1)(G2),
—N(G3)(G3), and
—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.
“Halogen atom” Specific examples of the “halogen atom” described in this specification (specific example group G11) include a fluorine atom, a chlorine atom, a bromine atom, an iodine atom, and the like.
“Substituted or unsubstituted fluoroalkyl group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.
“Substituted or unsubstituted haloalkyl group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.
“Substituted or unsubstituted alkoxy group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.
“Substituted or unsubstituted alkylthio group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.
“Substituted or unsubstituted aryloxy group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.
“Substituted or unsubstituted arylthio group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.
“Substituted or unsubstituted trialkylsilyl group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.
“Substituted or unsubstituted aralkyl group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

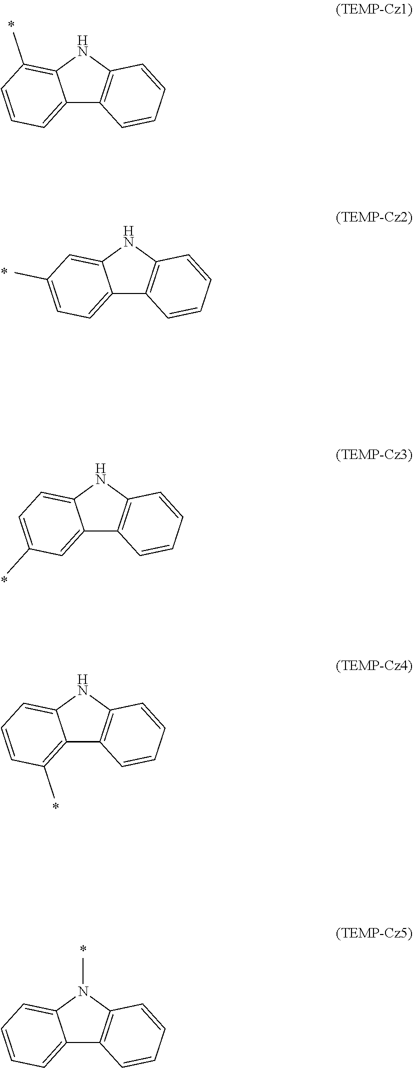

In this specification, the (9-phenyl)carbazolyl group is specifically any of the following groups, unless otherwise specified in this specification.

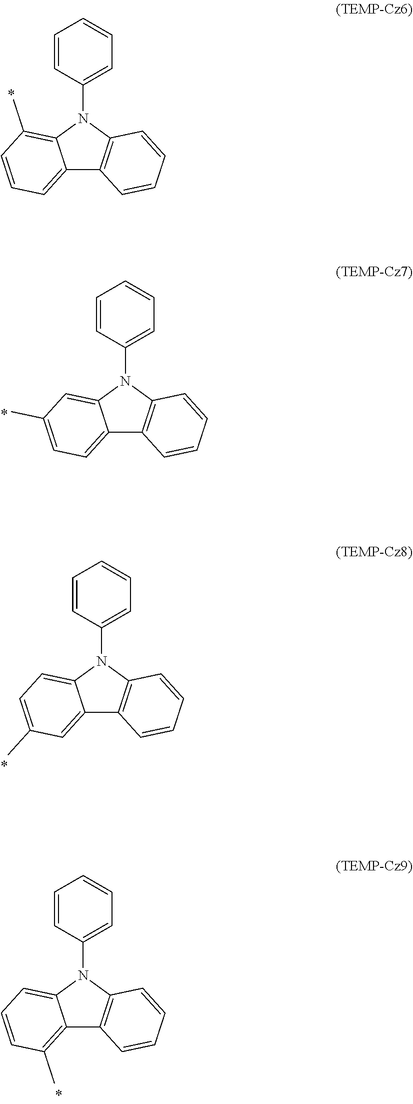 [SMILES: Cc1cccc2c3ccccc3n(-c3ccccc3)c12 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

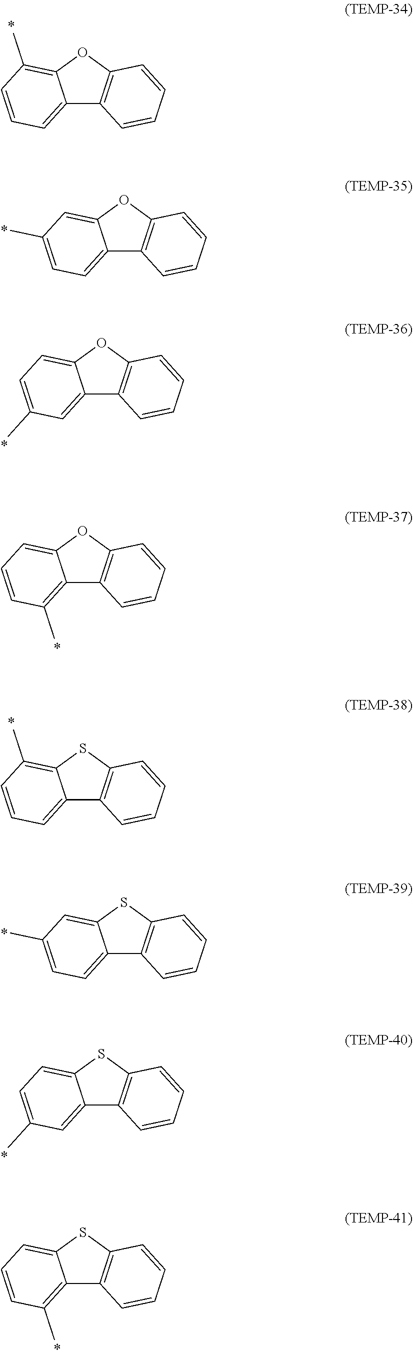

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.
“Substituted or unsubstituted arylene group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.
“Substituted or unsubstituted divalent heterocyclic group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.
“Substituted or unsubstituted alkylene group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

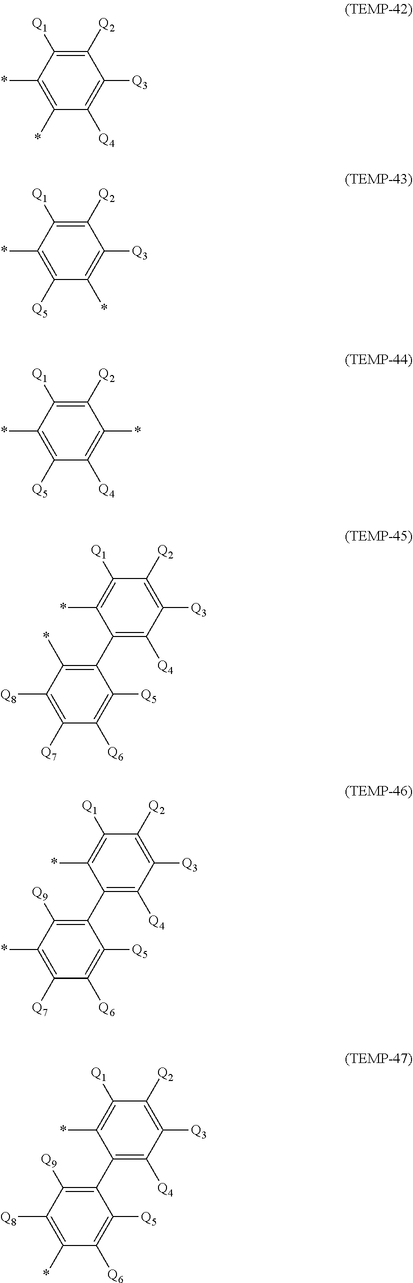

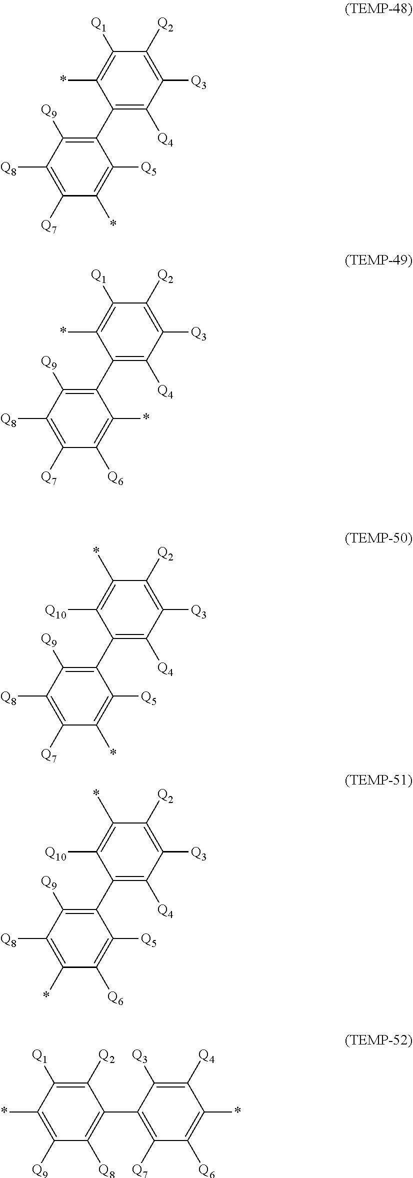

In the general formulas (TEMP-42) to (TEMP-52), Q₁ to Q₁₀ are independently a hydrogen atom or a substituent.

In the general formulas (TEMP-42) to (TEMP-52), * represents a bonding site.

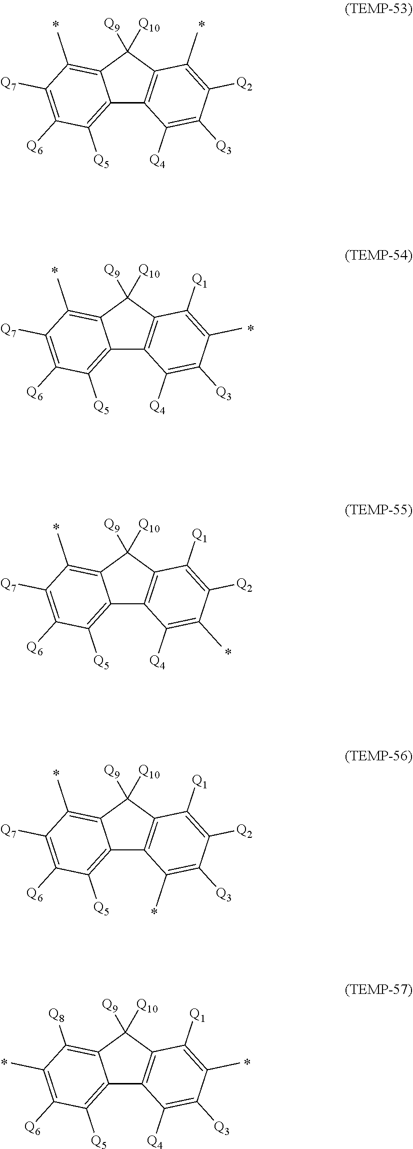

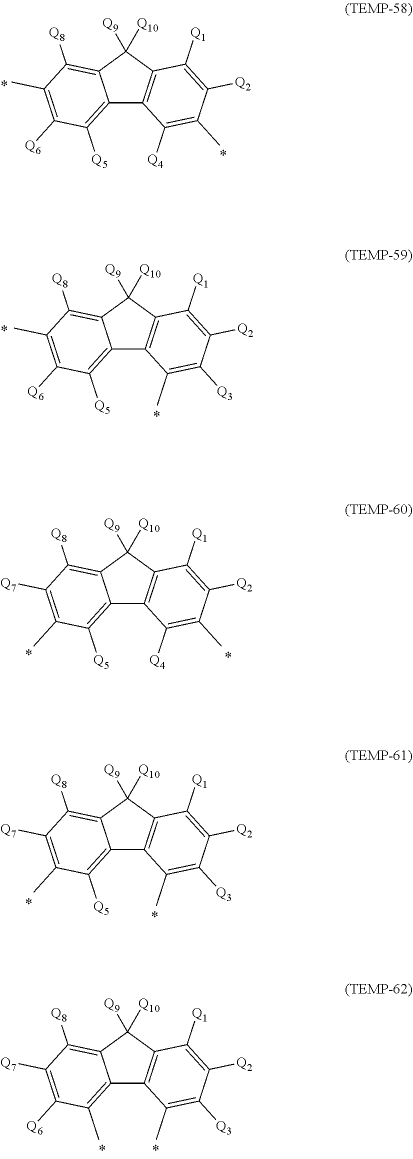

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

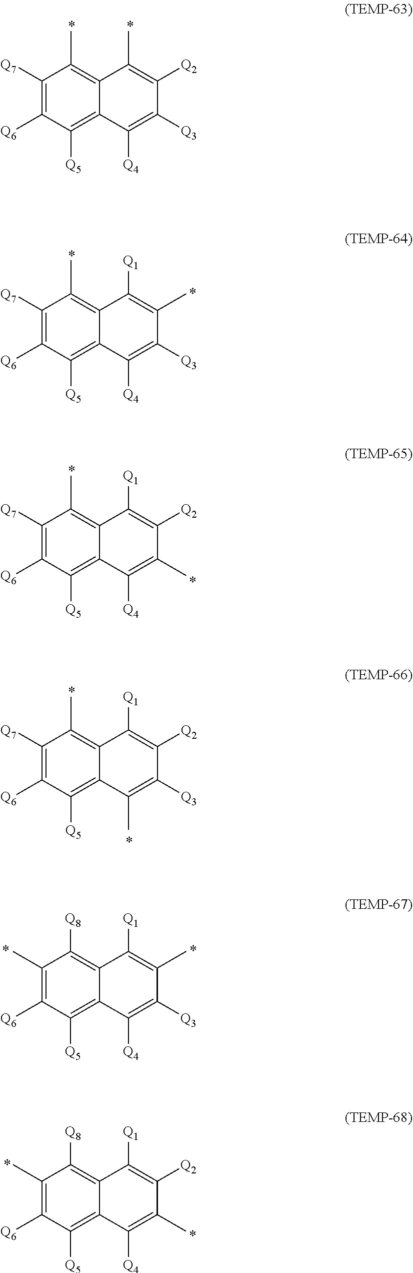

In the general formulas (TEMP-63) to (TEMP-68), Q to Q; are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

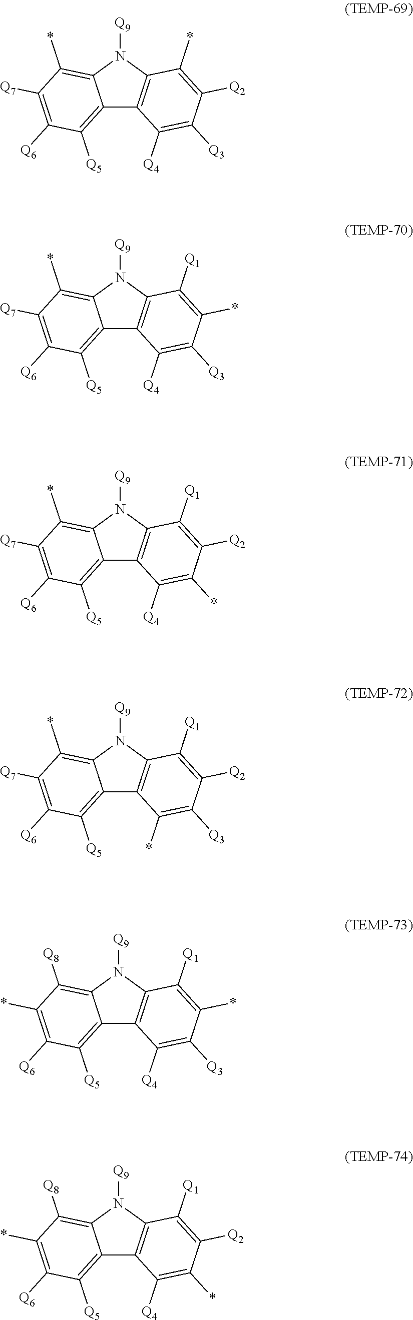

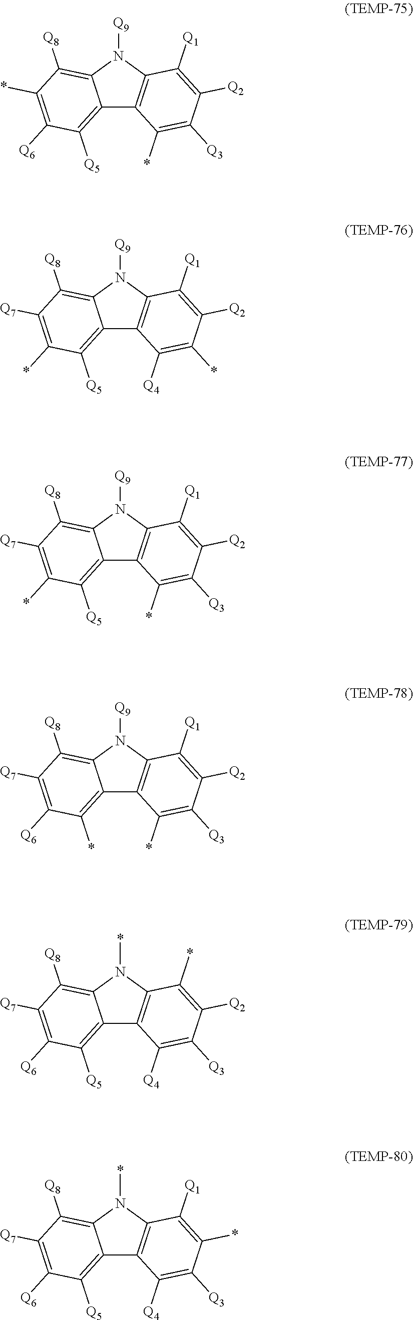

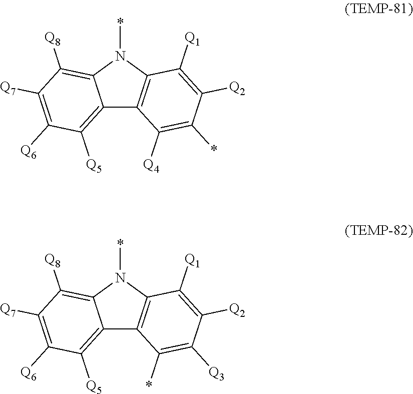

In the general formulas (TEMP-69) to (TEMP-82), Q₁ to Q₉ are independently a hydrogen atom or a substituent.

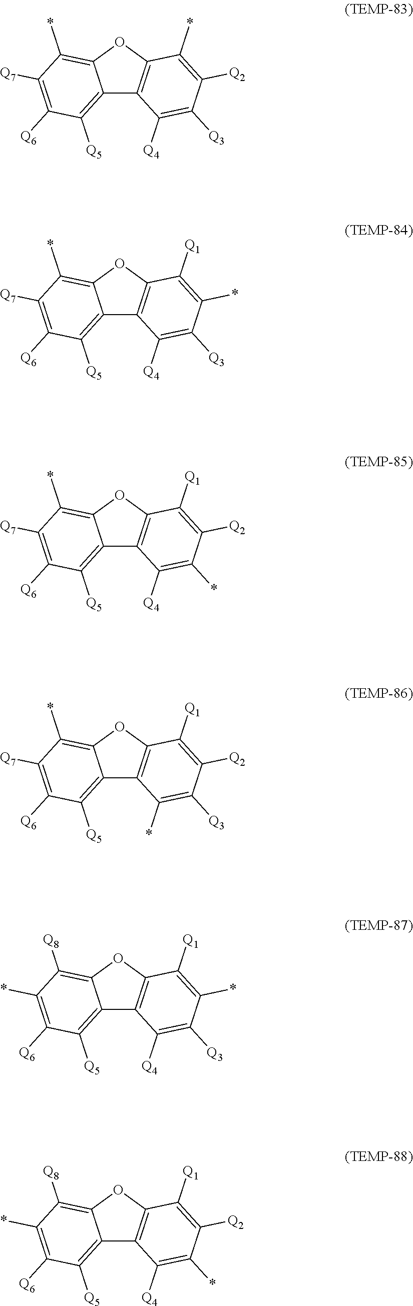

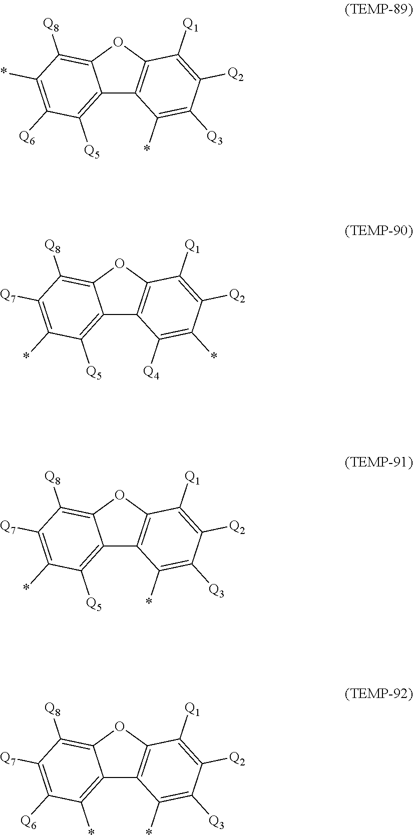

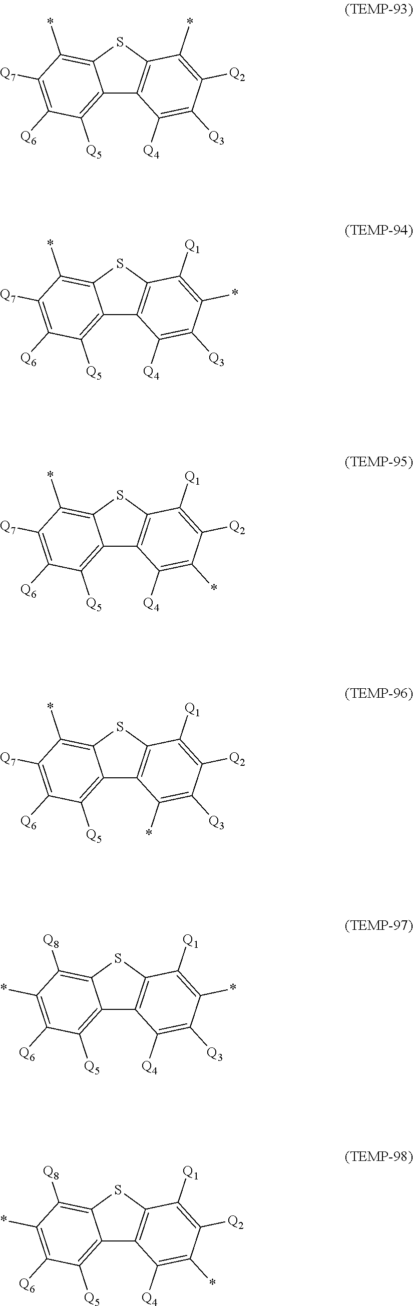

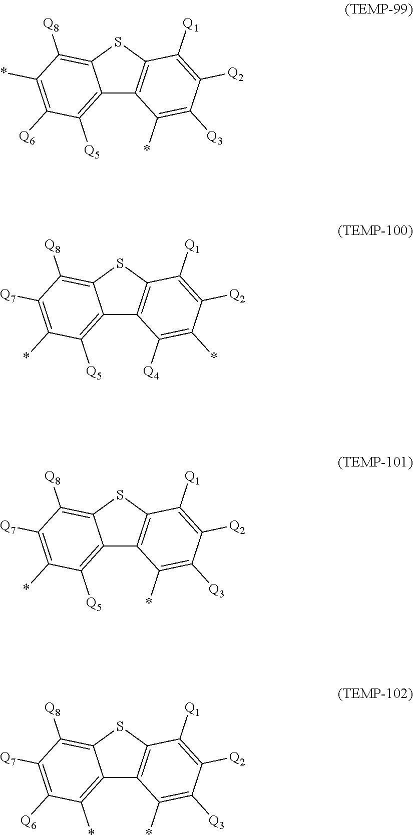

In the general formulas (TEMP-83) to (TEMP-102), Q₁ to Q₈ are independently a hydrogen atom or a substituent.

The above is the explanation of the “Substituent described in this specification.”
“The case where bonded with each other to form a ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

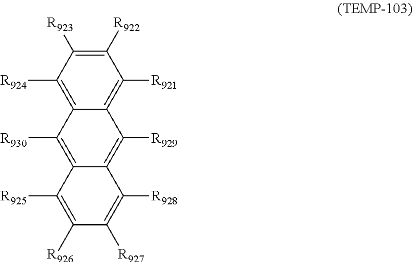

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₃, a pair of R₉₂₃ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring QB by bonding with each other, the anthracene compound represented by the general formula (TEMP-103) is represented by the following general formula (TEMP-104).

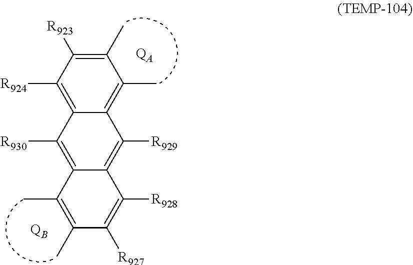

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Qc by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Qc share R₉₂₂.

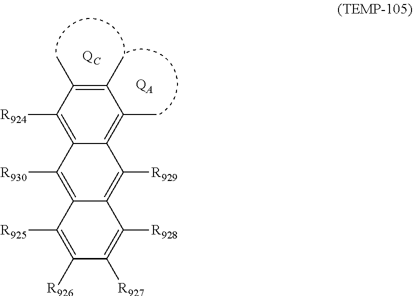

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring QB formed in the general formula (TEMP-104) are independently a “monocycle” or a “fused ring.” The ring Q_{A} and the ring Qc formed in the general formula (TEMP-105) are “fused ring.” The ring Q_{A} and ring Qc of the general formula (TEMP-105) are fused ring by fusing the ring Q_{A} and the ring Qc together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

Substituent in the case of “substituted or unsubstituted” In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁, to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Organic Electroluminescence Device]

An organic EL device according to an aspect of the present invention includes a cathode, an anode, an emitting layer arranged between the cathode and the anode, and an electron-transporting region arranged between the emitting layer and the cathode, wherein the electron-transporting region includes one or more compounds selected from the group consisting of compounds represented by the following formulas (1) to (4) and a rare earth element, and the electron-transporting region substantially does not include an alkali metal, a compound containing an alkali metal, a metal belonging to Group 13 of the Periodic Table of the Elements, and a compound having a metal belonging to Group 13 of the Periodic Table of the Elements.

When the organic EL device according to an aspect of the present invention includes the configurations described above, it can exhibit high device performance without using a material having high electron-transporting property such as Liq, Alq3 and Balq in the electron-transporting region. Further, since it is not required to form a mixed layer using such a material, a co-depositing step is not needed. As a result, it allows process simplicity to be greatly improved.

Each configuration of the organic EL device according to an aspect of the present invention will be described below.

(Electron-Transporting Region) The electron-transporting region indicates a general term for one or two or more layers arranged between the cathode and the emitting layer. The electron-transporting region is configured, for example, from each layer which is referred to as a hole-barrier layer, an electron-transporting layer and an electron-injecting layer from the emitting layer side, and it may be a stacked structure including all of them, or may be a layer configuration merely including a part of them. Further, each of the above layers may be formed by using two or more kinds of layers, and for example, two kinds of electron-transporting layers having different compositions may be stacked, or two kinds of electron-injecting layers having different compositions may be stacked.

Stacked structures of the electron-transporting region in an aspect of the present invention are shown below.
(a) (an emitting layer/) a first layer (an electron-transporting layer)/a second layer (an electron-injecting layer) (/a cathode)
(b) (an emitting layer/) a third layer (a hole-barrier layer)/a first layer (an electron-transporting layer)/a second layer (an electron-injecting layer) (/a cathode)
(c) (an emitting layer /) a third layer (a hole-barrier layer)/a fourth layer (a second electron-transporting layer)/a first layer (a first electron-transporting layer)/a second layer (an electron-injecting layer) (/a cathode)

In an aspect of the present invention, the second layer (electron-injecting layer) is arranged nearest on the cathode side in the electron-transporting region.

The above (b) can be expressed as a configuration that the electron-transporting region includes a third layer between the emitting layer and the first layer, and the electron-transporting region does not include other layers between the emitting layer and the third layer.

The above (c) can be expressed as a configuration that the electron-transporting region includes a fourth layer between the third layer and the first layer.

### (Alkali Metal and the Like)

In the organic EL device according to an aspect of the present invention, the electron-transporting region substantially does not include an alkali metal, a compound containing an alkali metal, a metal belonging to Group 13 of the Periodic Table of the Elements, and a compound having a metal belonging to Group 13 of the Periodic Table of the Elements (hereinafter, also referred to as the “specific metal and specific compound”).

The expression “substantially does not include” refers that the electron-transporting region does not include any specific metal and specific compound or includes at least a part of the specific metal and specific compound as long as the effects of the present invention are not impaired. For example, the electron-transporting region may include at least a part of the specific metal and specific compound contained in a commercially available material (for example, a rare earth element material) as unavoidable impurities.

The amounts (total amount) of the specific metal and specific compound is, for example, 1% by mass or less, 0.5% by mass or less, 0.1% by mass or less, or 0.01% by mass or less in the electron-transporting region. The amounts thereof can be calculated from the total amount of the specific metal and specific compound contained in each material used in formation of the electron-transporting region and the mass of electron-transporting region. Further, Secondary Ion Mass Spectrometry (SIMS analysis) can be used for a measurement of the amounts in the organic EL device being a finished product.

The specific metal and specific compound will be specifically described.

Examples of the alkali metal include lithium, sodium, potassium, rubidium, cesium, and francium.

As the compound containing an alkali metal, fluoride of an alkali metal, oxide thereof and other compounds thereof can be given. Examples thereof include lithium fluoride, lithium oxide, 8-hydroxyquinolinolato-lithium (Liq), cesium fluoride, and the like.

Examples of the metal belonging to Group 13 of the Periodic Table of the Elements include boron, aluminum, gallium, indium and thallium.

As the compound having a metal belonging to Group 13 of the Periodic Table of the Elements, a complex compound of the metal and the like can be given. Examples thereof include tris (8-quinolinolato) aluminum (Alq3), tris(4-methyl-8-quinolinolato)aluminum (also referred to as an “Almq3”), bis(2-methyl-8-quinolinolato)(4-phenylphenolato)aluminum (BAlq), and the like.

### (Compounds of Formulas (1) to (4) and Rare Earth Element)

The organic EL device according to an aspect of the present invention includes one or more compounds selected from the group consisting of compounds represented by the formulas (1) to (4) and a rare earth element in the electron-transporting region. A containing aspect of these materials is not particularly limited, and a layer containing the former and a layer containing the latter may be different layer, or both of them may be included in the specific one layer. The organic EL device according to an aspect of the present invention preferably includes one or more compounds selected from the group consisting of compounds represented by the formulas (1) to (4) in the first layer (electron-transporting layer) of the above-mentioned layer configuration, and the rare earth element in the second layer (electron-injecting layer) thereof.

In such a case, a part of rare earth element included in the second layer may be doped in the first layer.

The rare earth element is scandium (Sc), yttrium (Y), and lanthanoid elements (lanthanum, cerium, praseodymium, neodymium, promethium, samarium, europium, gadolinium, terbium, dysprosium, holmium, erbium, thulium, ytterbium and lutetium).

In an aspect of the present invention, the rare earth element included in the electron-transporting region is Yb.

Each compound represented by the formulas (1) to (4) will be specifically described below.

### [Compound Represented by Formula (1)]

A compound represented by the formula (1) is shown as follows:

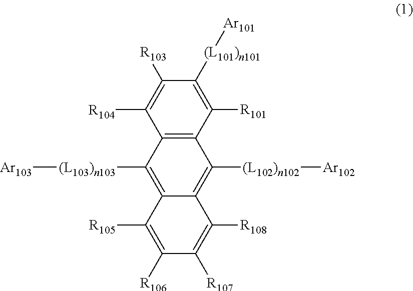

wherein in the formula (1),
R₁₀₁ and R₁₀₃ to R₁₀₈ are independently a hydrogen atom, or a substituent R;
L₁₀₁ is
a single bond,
a substituted or unsubstituted arylene group having 6 to 50 ring carbon atoms, or
a substituted or unsubstituted divalent heterocyclic group having 5 to 50 ring atoms;
L₁₀₂ and L₁₀₃ are independently
a single bond, or
a substituted or unsubstituted arylene group having 6 to 50 ring carbon atoms;
Ar₁₀₁ is
a substituted or unsubstituted monovalent group including a nitrogen-containing six-membered ring, or
a substituted or unsubstituted benzimidazolyl group;
Ar₁₀₂ and Ar₁₀₃ are independently a substituted or unsubstituted aryl group having 6 to 50 ring carbon atoms;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
n101 is an integer of 1 to 3; when n101 is 2 or more, two or more L₁₀₁'s may be the same as or different from each other;
n102 is an integer of 1 to 3; when n102 is 2 or more, two or more L₁₀₂'s may be the same as or different from each other; and
n103 is an integer of 1 to 3; when n103 is 2 or more, two or more L₁₀₃'s may be the same as or different from each other.

When all of L₁₀₁'s is a single bond in the formula (1), Ar₁₀₁ is directly bonded with the anthracene skeleton.

When all of L₁₀₂'s is a single bond in the formula (1), Ar₁₀₂ is directly bonded with the anthracene skeleton.

When all of L₁₀₃'s is a single bond in the formula (1), Ar₁₀₃ is directly bonded with the anthracene skeleton.

In one embodiment, the compound represented by the formula (1) is a compound represented by the following formula (1-1):

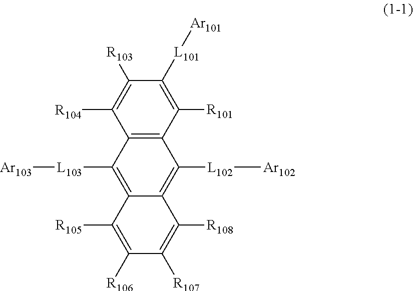

wherein in the formula (1-1),
R₁₀₁ and R₁₀₃ to R₁₀₈, L₁₀ to L₁₀₃, and Ar₁₀₁ to Ar₁₀₃ are the same as defined in the formula (1).

In one embodiment, L₁₀₂ and L₁₀₃ are single bonds.

In one embodiment, R₁₀₁ and R₁₀₃ to R₁₀₈ are hydrogen atoms.

In one embodiment, the compound represented by the formula (1) is a compound represented by the following formula (1-11):

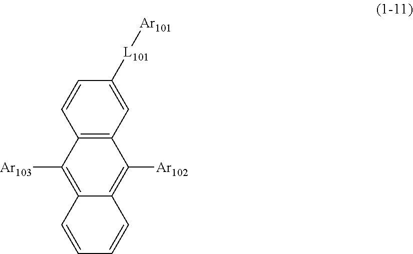

wherein in the formula (1-11),
L₁₀₁, and Ar₁₀₁ to Ar₁₀₃ are the same as defined in the formula (1).

In one embodiment, a substituent in the case of “substituted or unsubstituted” in the formula (1) is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,
—Si(R₄₁)(R₄₂)(R₄₃),
—C(═O)R₄₄, —COOR₄₅,
—S(═O)₂R₄₆,
—P(═O)(R₄₇)(R₄₈),
—Ge(R₄₉)(R₅₀)(R₅₁),
—N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other),
a hydroxy group,
a halogen atom,
a cyano group,
a nitro group,
an aryl group having 6 to 50 ring carbon atoms, and
a monovalent heterocyclic group having 5 to 50 ring atoms.

In one embodiment, a substituent in the case of “substituted or unsubstituted” in the formula (1) is selected from the group consisting of
an alkyl group having 1 to 18 carbon atoms,
an aryl group having 6 to 18 ring carbon atoms, and
a monovalent heterocyclic group having 5 to 18 ring atoms.

The compound represented by the formula (1) can be synthesized by using known alternative reactions or raw materials adapted to the target compound.

Specific examples of the compound represented by the formula (1) will be described below, but these are merely examples, and the compound represented by the formula (1) is not limited to the following specific examples.

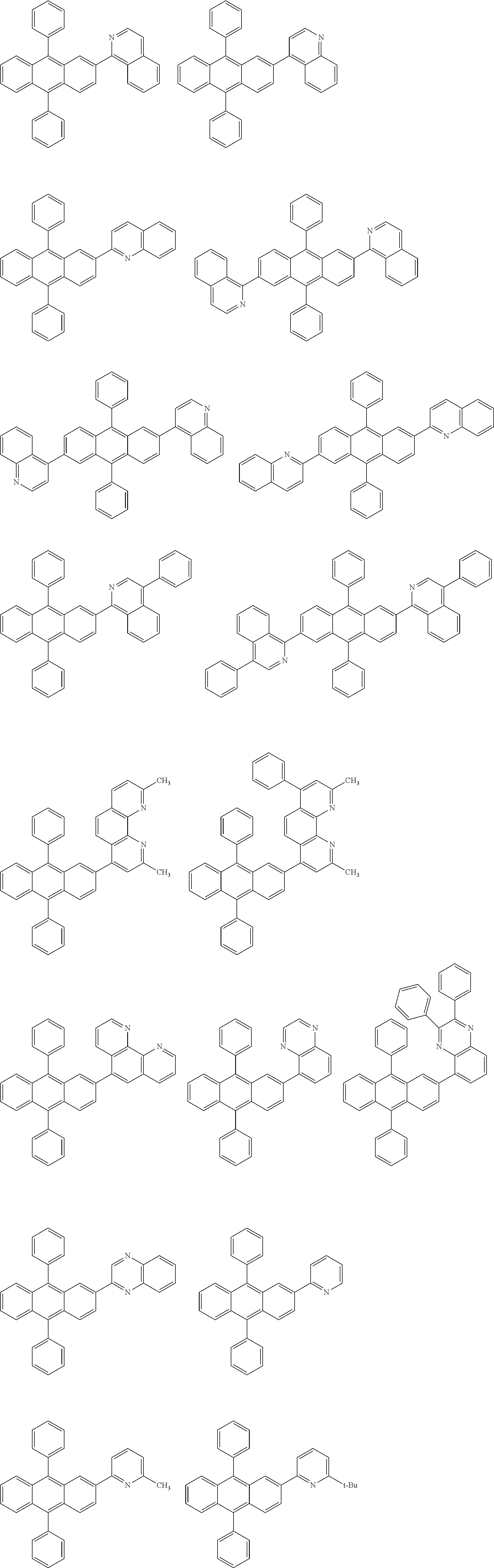

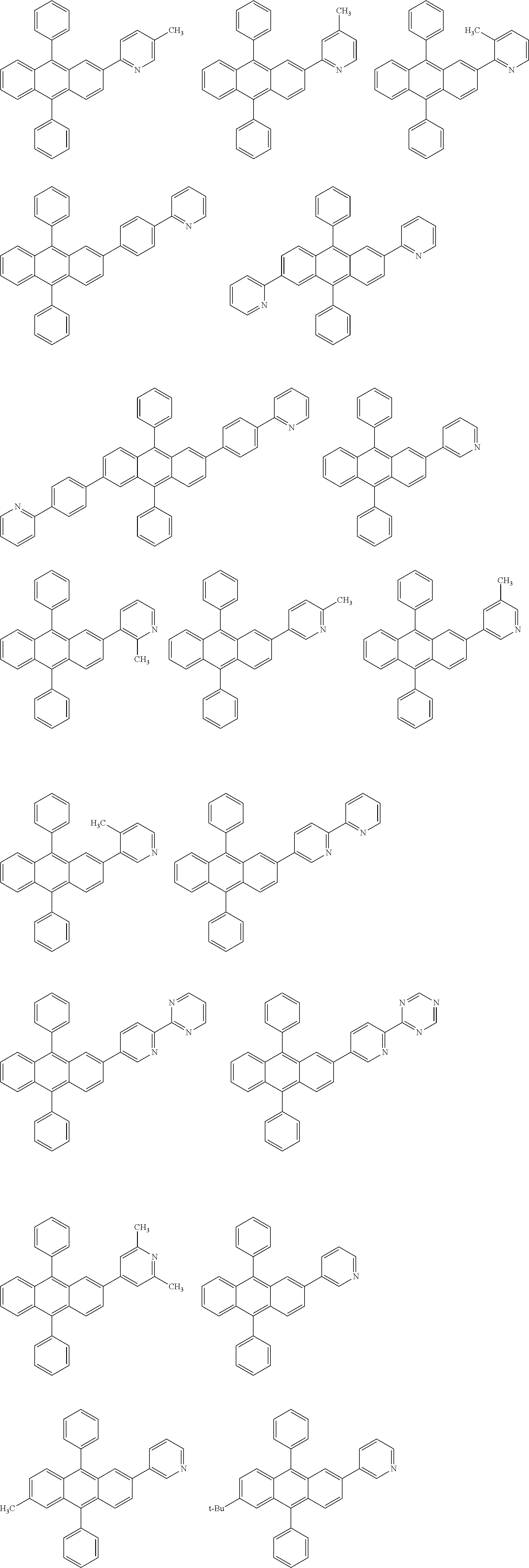

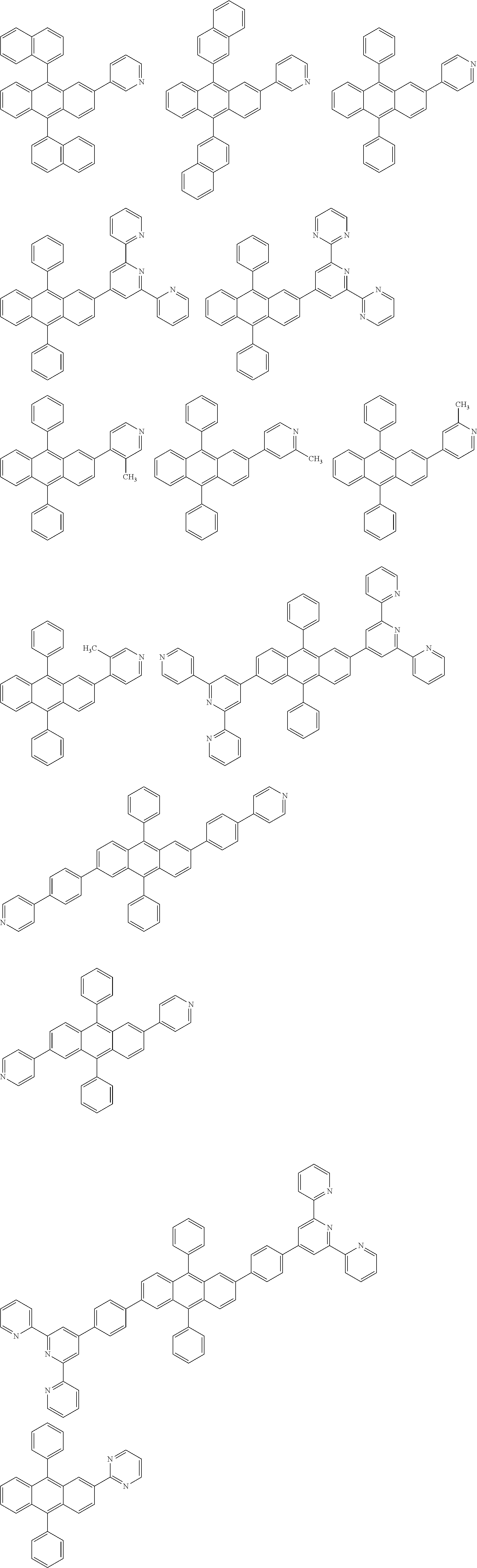

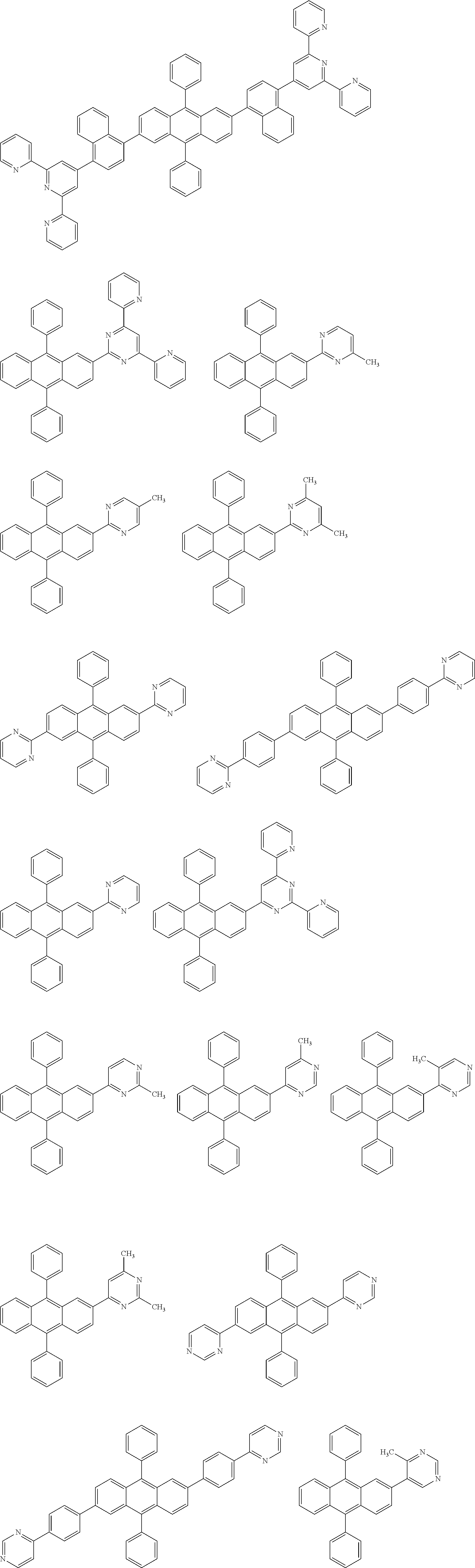

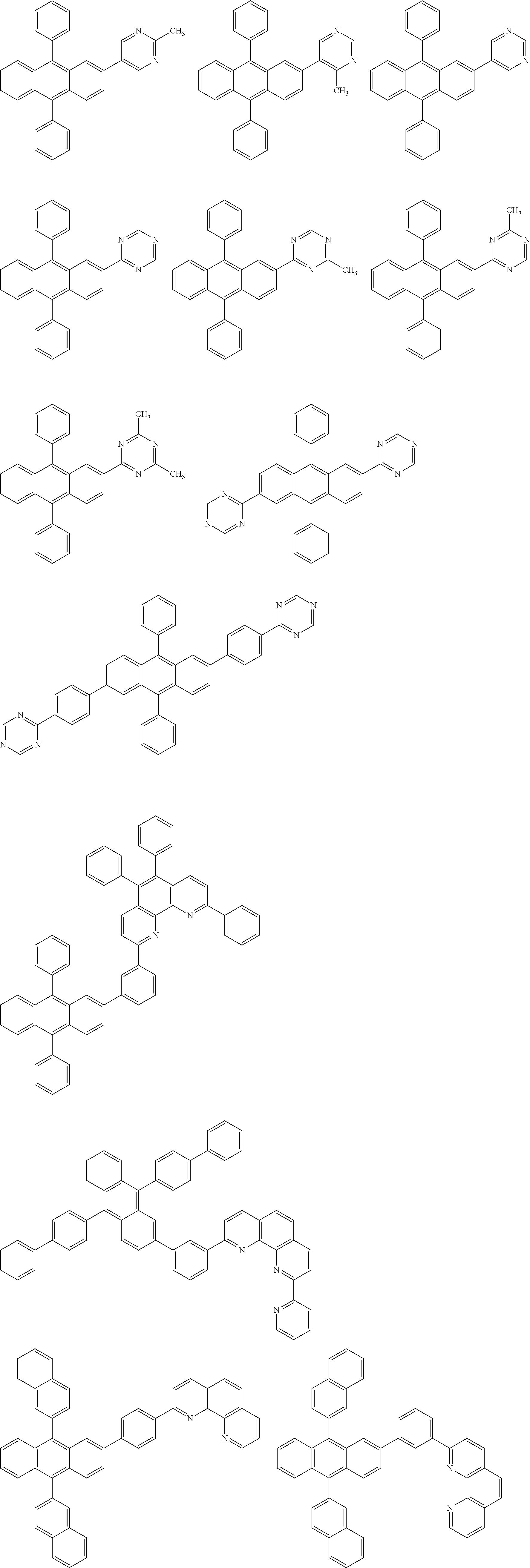

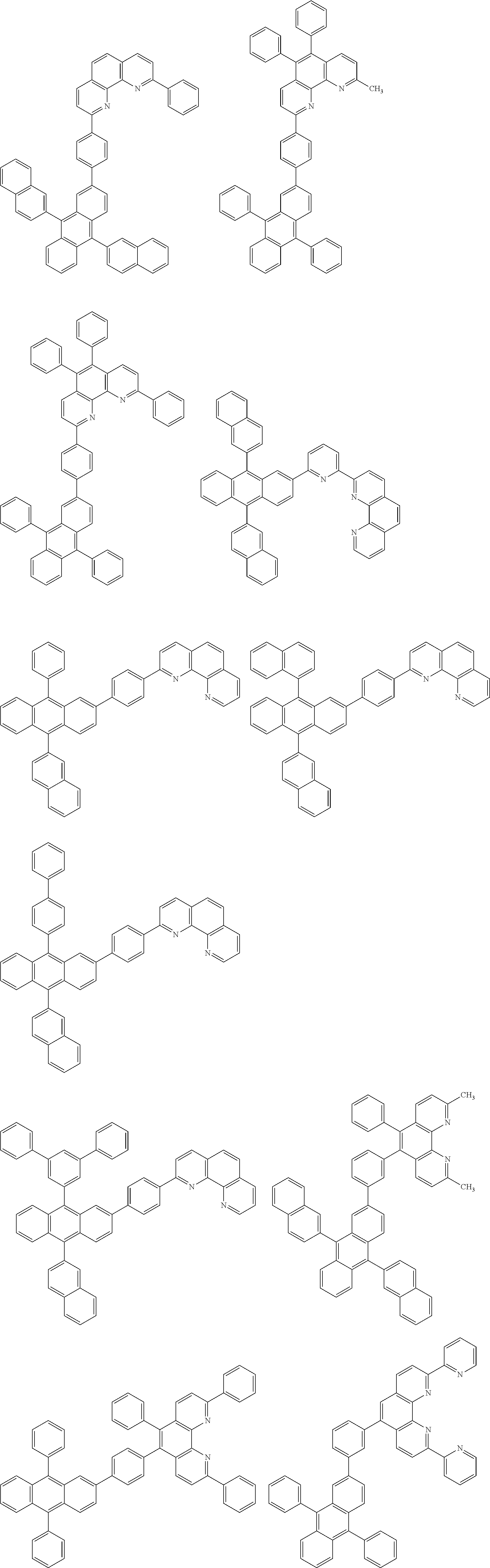

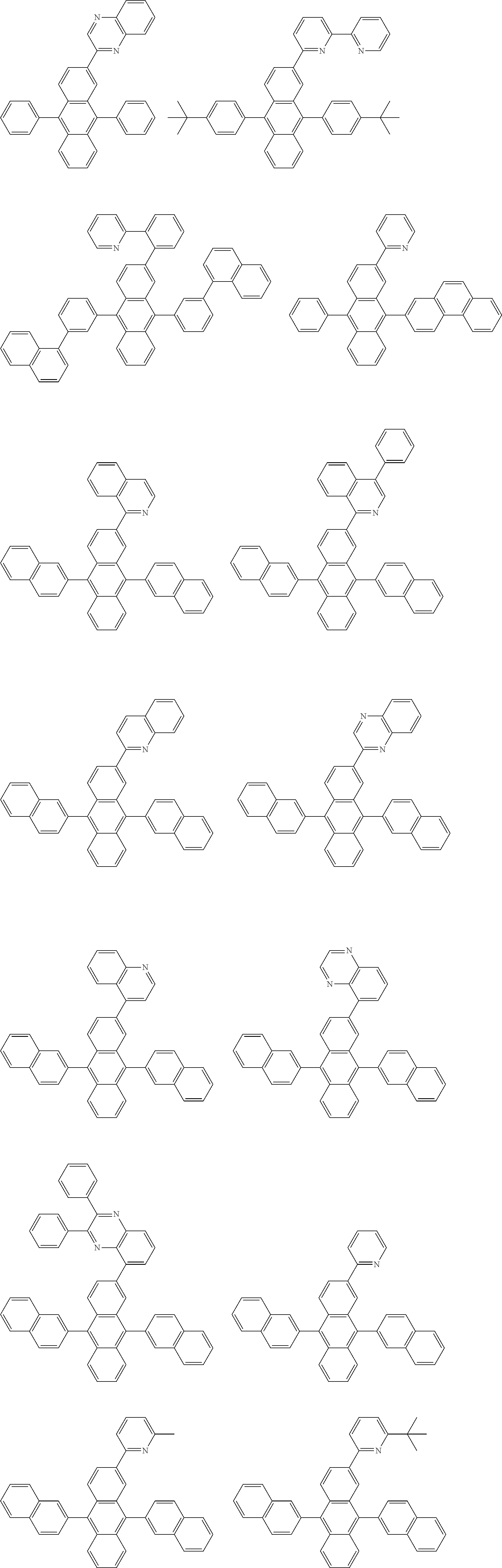

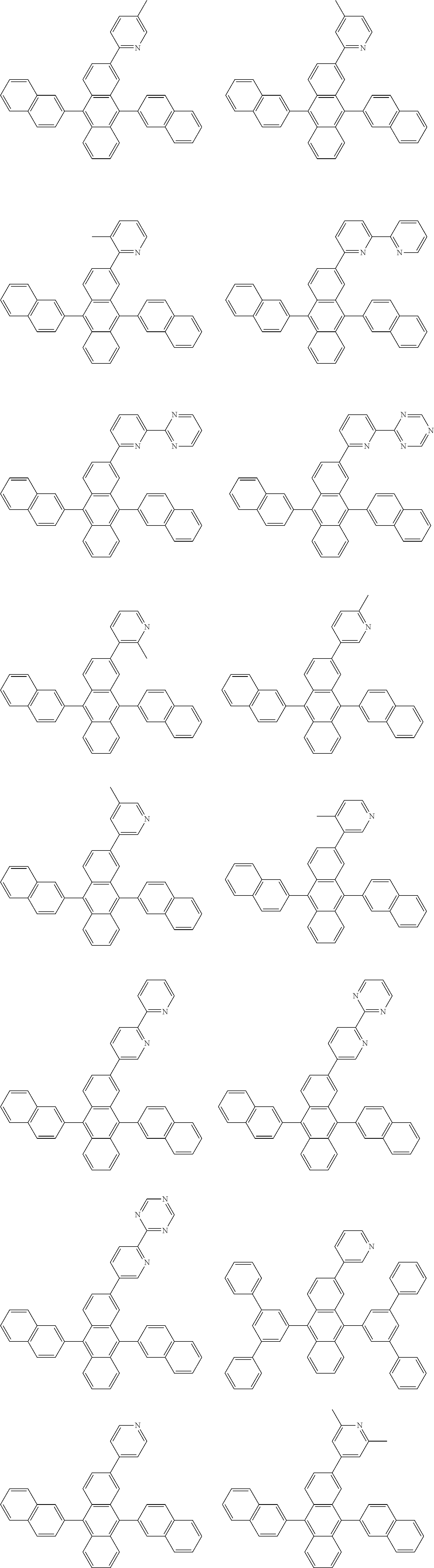

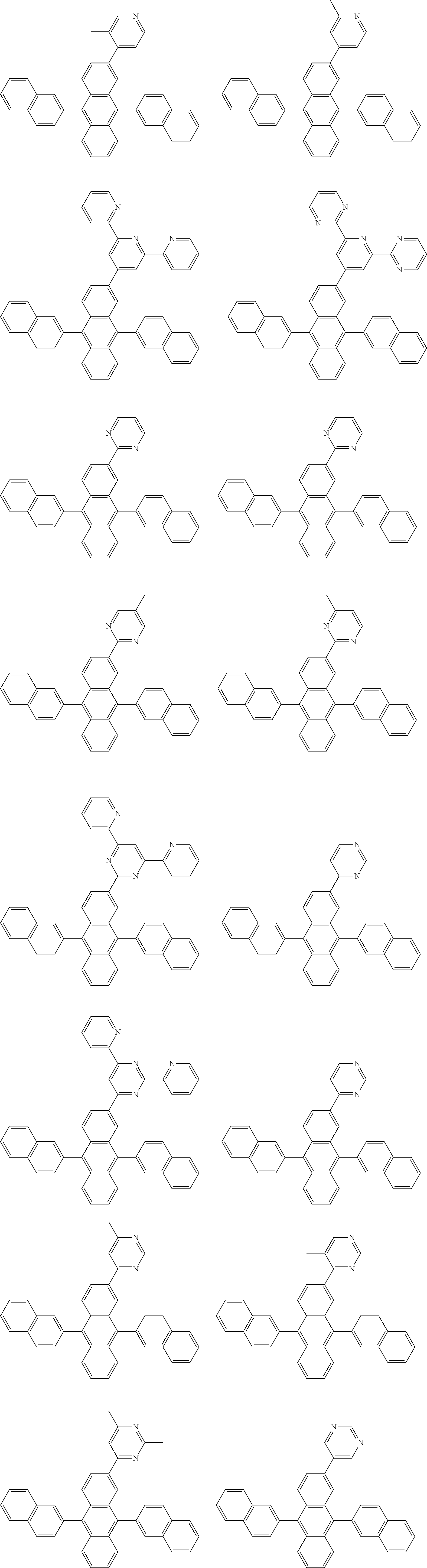

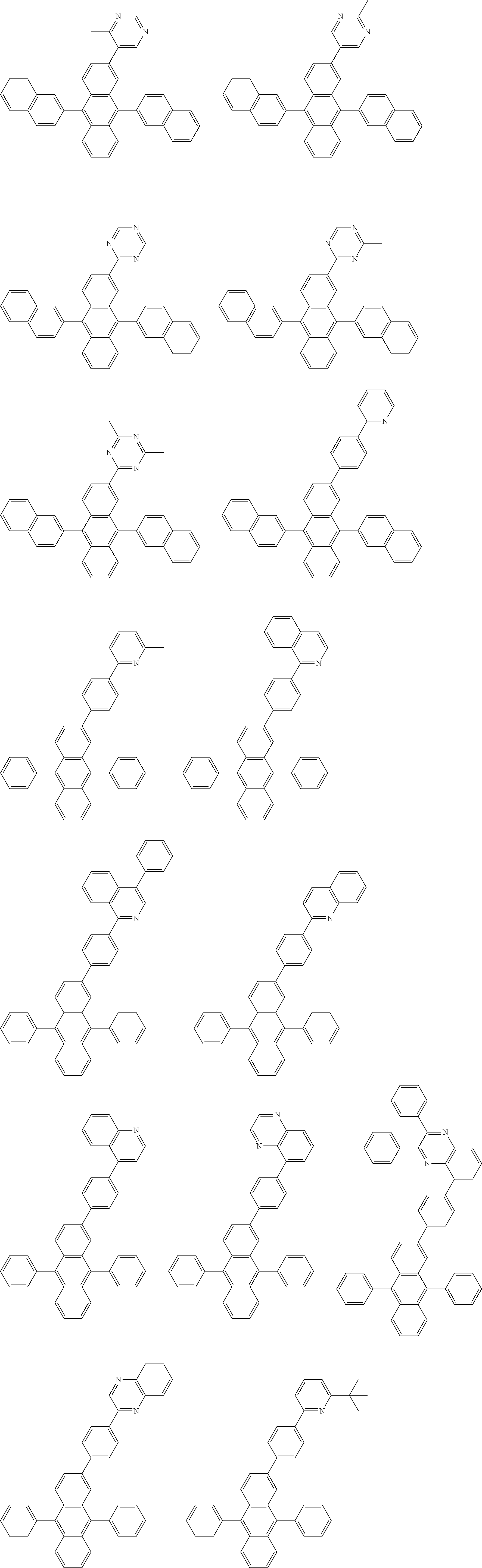

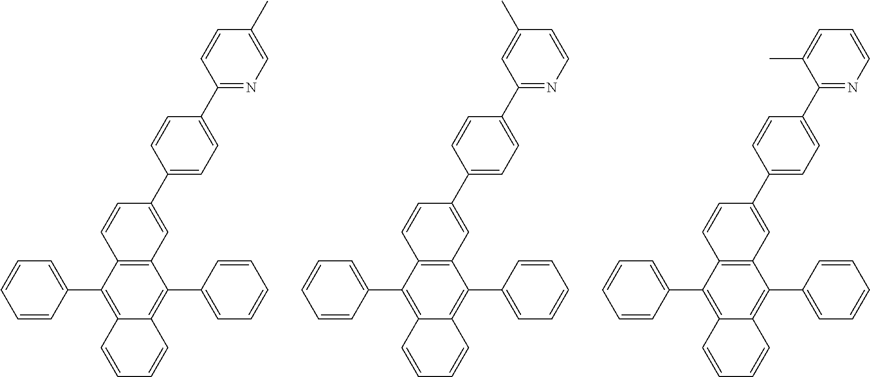

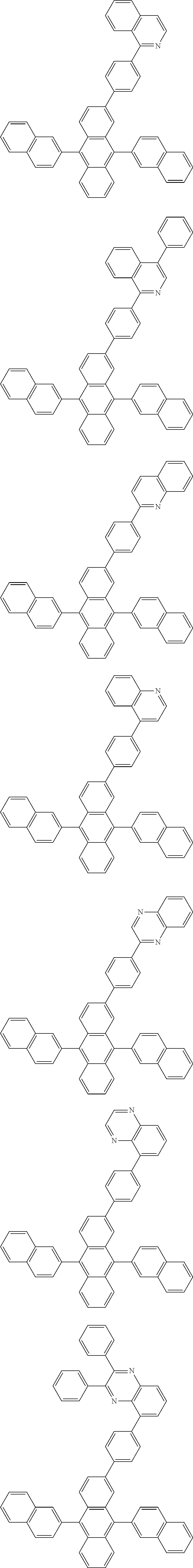

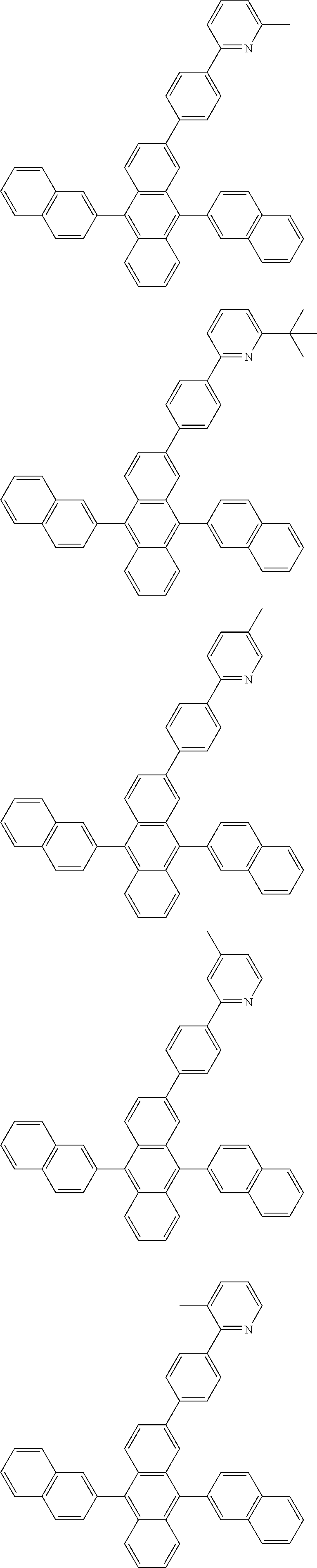

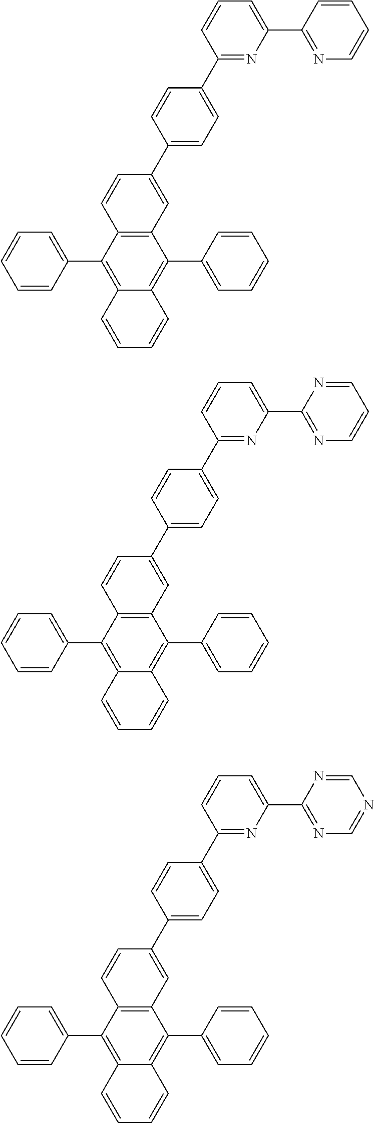 [SMILES: c1ccc(-c2c3ccccc3c(-c3ccccc3)c3cc(-c4ccc(-c5cccc(-c6ccccn6)n5)cc4)ccc23)cc1 ; c1ccc(-c2c3ccccc3c(-c3ccccc3)c3cc(-c4ccc(-c5cccc(-c6ncccn6)n5)cc4)ccc23)cc1 ; c1ccc(-c2c3ccccc3c(-c3ccccc3)c3cc(-c4ccc(-c5cccc(-c6ncncn6)n5)cc4)ccc23)cc1]

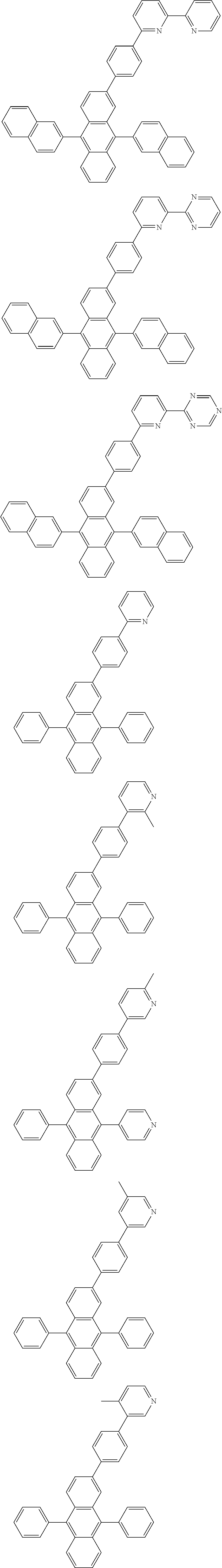

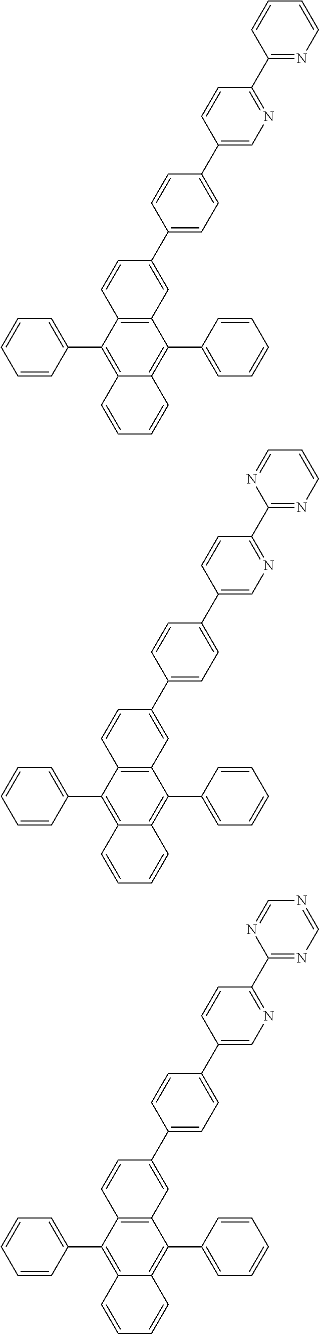 [SMILES: c1ccc(-c2c3ccccc3c(-c3ccccc3)c3cc(-c4ccc(-c5ccc(-c6ccccn6)nc5)cc4)ccc23)cc1 ; c1ccc(-c2c3ccccc3c(-c3ccccc3)c3cc(-c4ccc(-c5ccc(-c6ncccn6)nc5)cc4)ccc23)cc1 ; c1ccc(-c2c3ccccc3c(-c3ccccc3)c3cc(-c4ccc(-c5ccc(-c6ncncn6)nc5)cc4)ccc23)cc1]

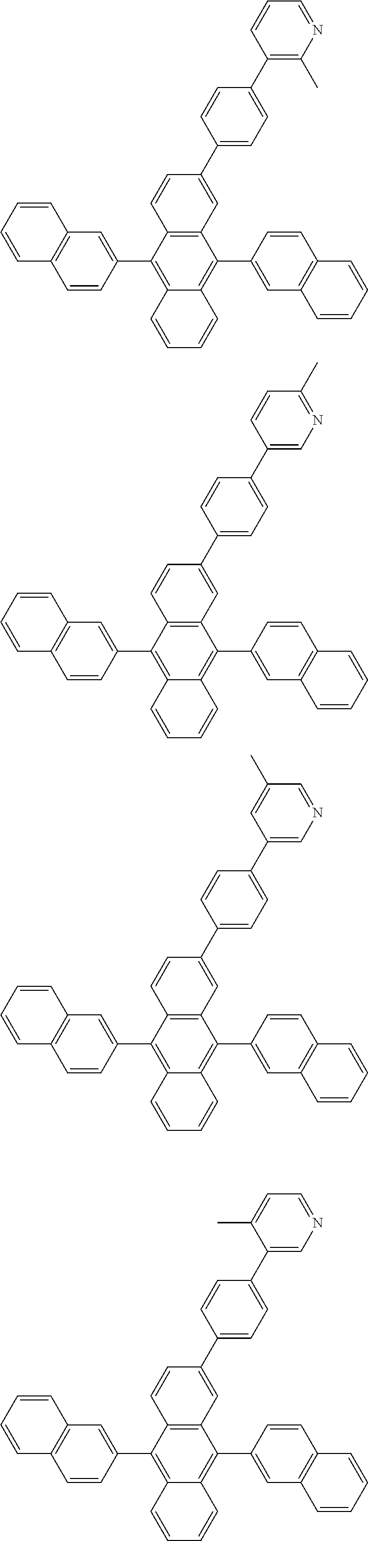

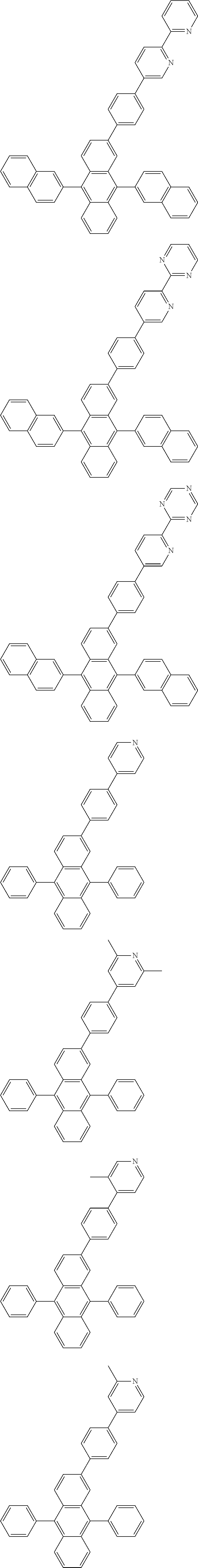

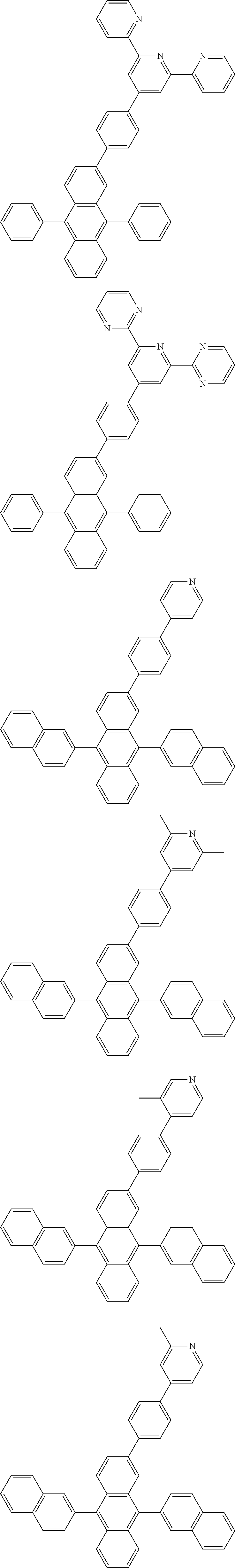

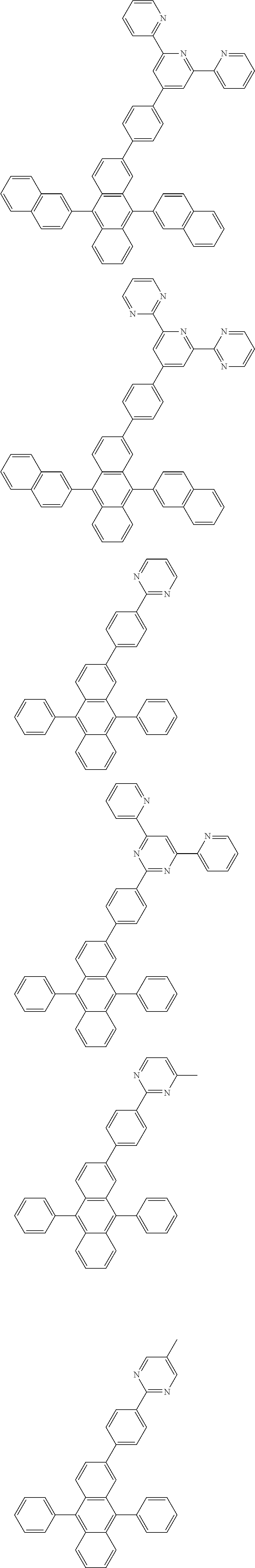

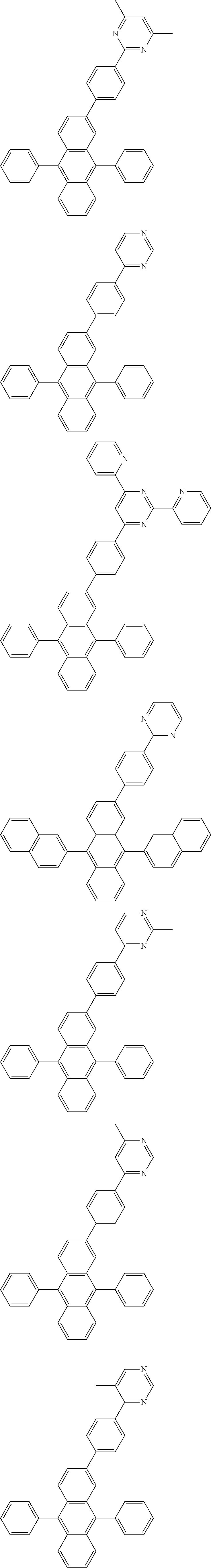

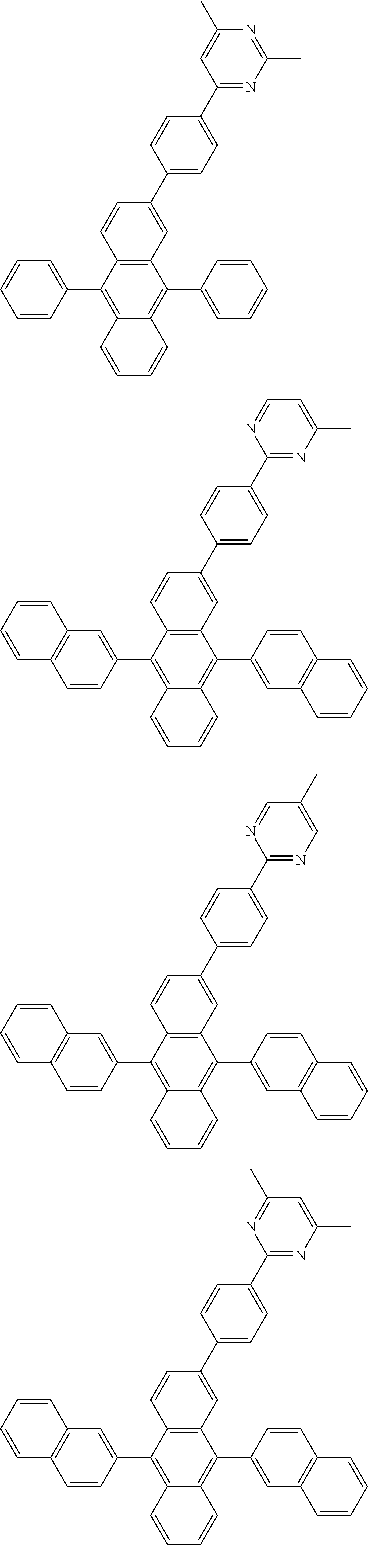 [SMILES: Cc1cc(-c2ccc(-c3ccc4c(-c5ccccc5)c5ccccc5c(-c5ccccc5)c4c3)cc2)nc(C)n1 ; Cc1ccnc(-c2ccc(-c3ccc4c(-c5ccc6ccccc6c5)c5ccccc5c(-c5ccc6ccccc6c5)c4c3)cc2)n1 ; Cc1cnc(-c2ccc(-c3ccc4c(-c5ccc6ccccc6c5)c5ccccc5c(-c5ccc6ccccc6c5)c4c3)cc2)nc1 ; Cc1cc(C)nc(-c2ccc(-c3ccc4c(-c5ccc6ccccc6c5)c5ccccc5c(-c5ccc6ccccc6c5)c4c3)cc2)n1]

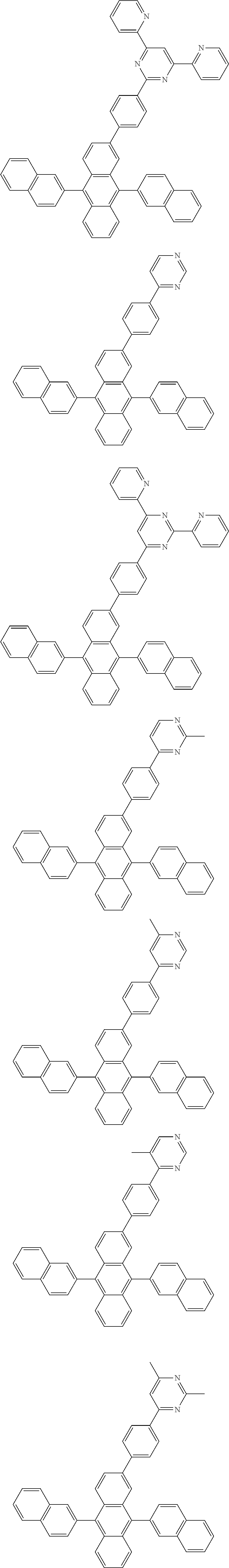

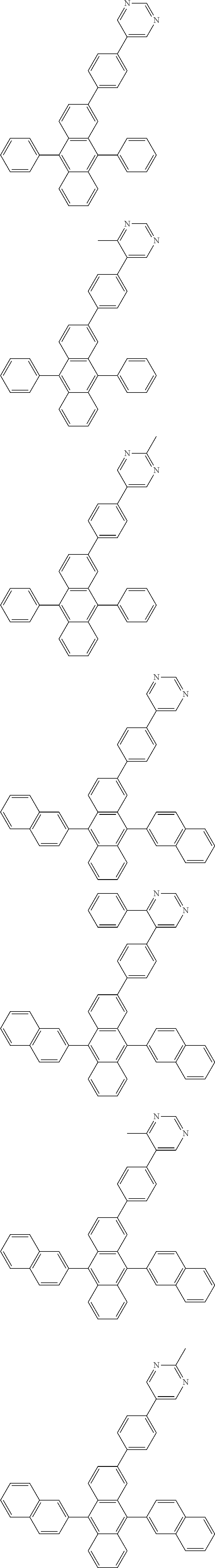

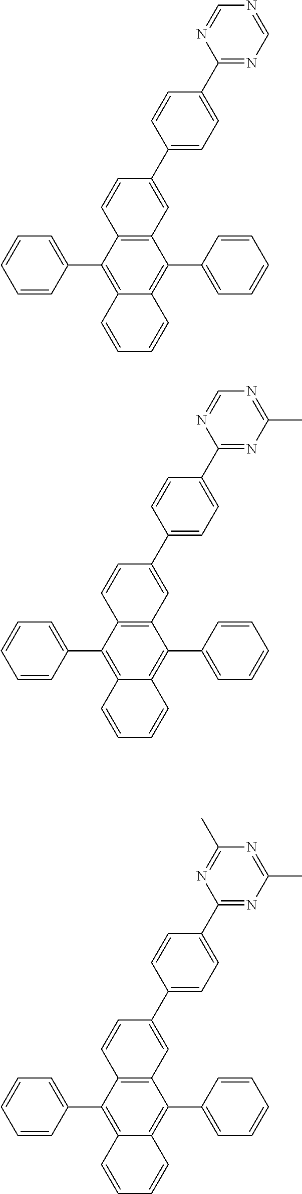 [SMILES: c1ccc(-c2c3ccccc3c(-c3ccccc3)c3cc(-c4ccc(-c5ncncn5)cc4)ccc23)cc1 ; Cc1ncnc(-c2ccc(-c3ccc4c(-c5ccccc5)c5ccccc5c(-c5ccccc5)c4c3)cc2)n1 ; Cc1nc(C)nc(-c2ccc(-c3ccc4c(-c5ccccc5)c5ccccc5c(-c5ccccc5)c4c3)cc2)n1]

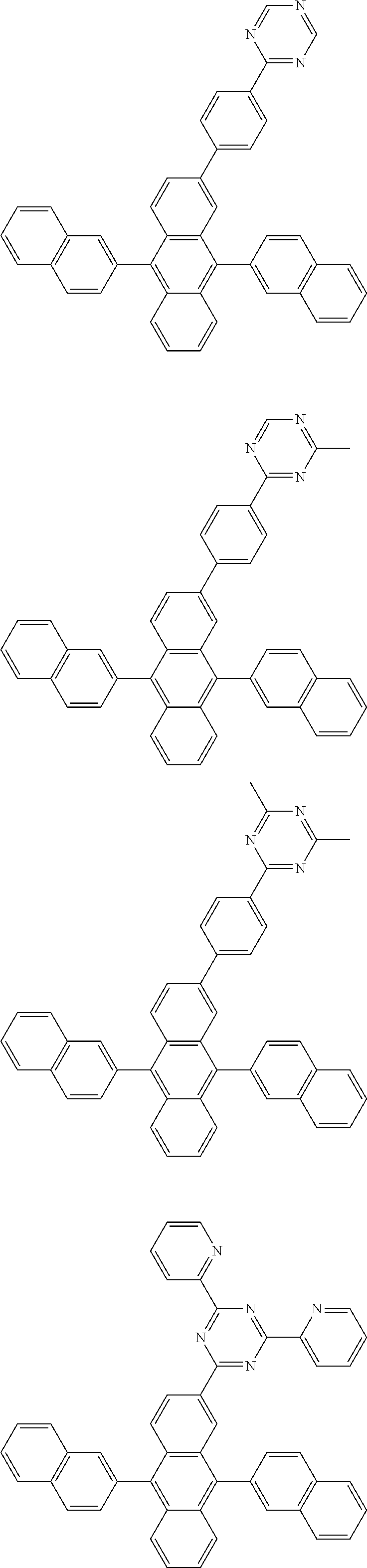 [SMILES: c1ccc2cc(-c3c4ccccc4c(-c4ccc5ccccc5c4)c4cc(-c5ccc(-c6ncncn6)cc5)ccc34)ccc2c1 ; Cc1ncnc(-c2ccc(-c3ccc4c(-c5ccc6ccccc6c5)c5ccccc5c(-c5ccc6ccccc6c5)c4c3)cc2)n1 ; Cc1nc(C)nc(-c2ccc(-c3ccc4c(-c5ccc6ccccc6c5)c5ccccc5c(-c5ccc6ccccc6c5)c4c3)cc2)n1 ; c1ccc(-c2nc(-c3ccc4c(-c5ccc6ccccc6c5)c5ccccc5c(-c5ccc6ccccc6c5)c4c3)nc(-c3ccccn3)n2)nc1]

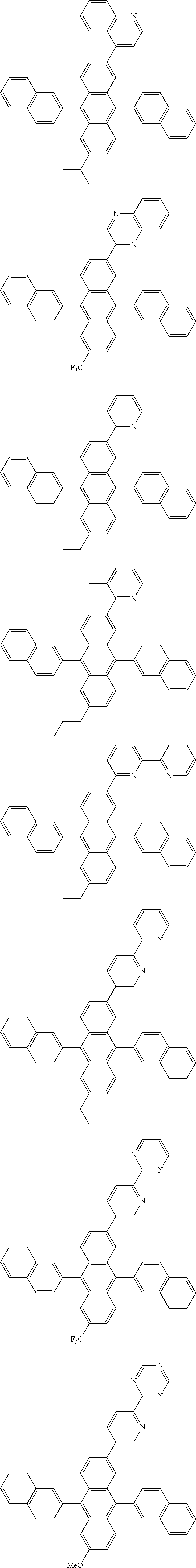

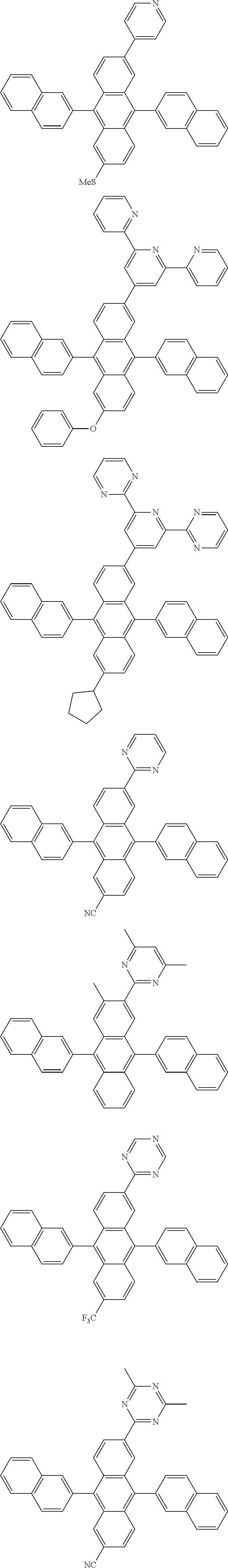

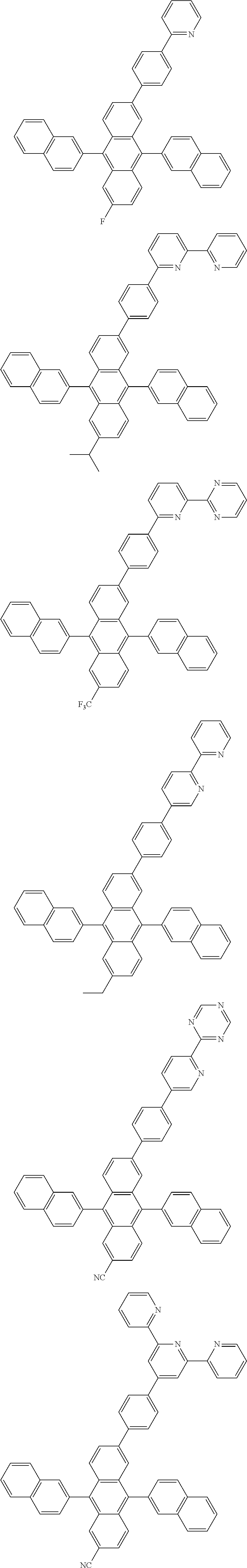

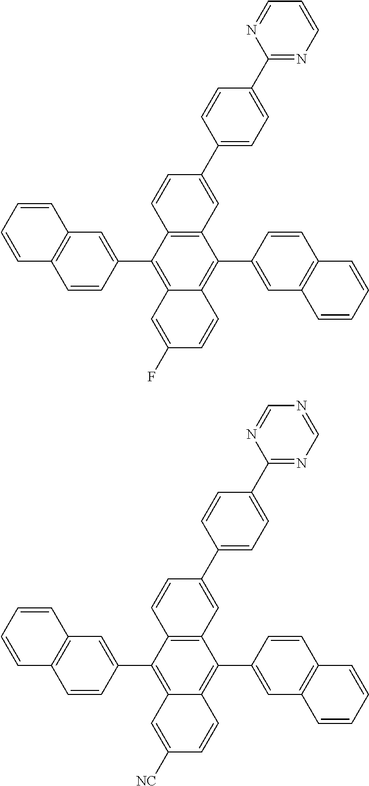 [SMILES: Fc1ccc2c(-c3ccc4ccccc4c3)c3cc(-c4ccc(-c5ncccn5)cc4)ccc3c(-c3ccc4ccccc4c3)c2c1 ; N#Cc1ccc2c(-c3ccc4ccccc4c3)c3cc(-c4ccc(-c5ncncn5)cc4)ccc3c(-c3ccc4ccccc4c3)c2c1]

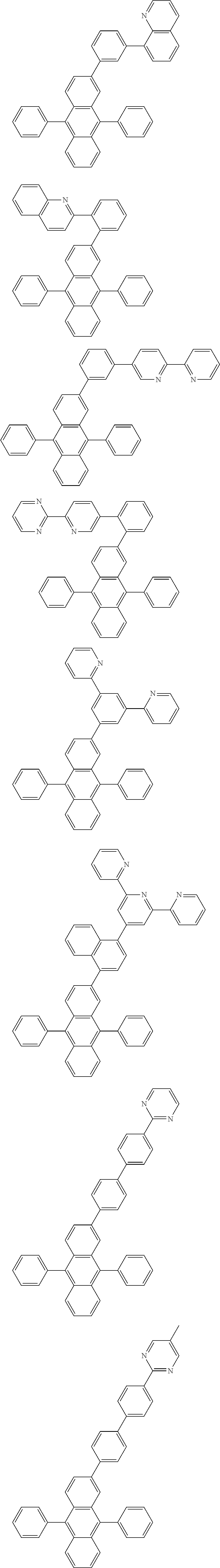

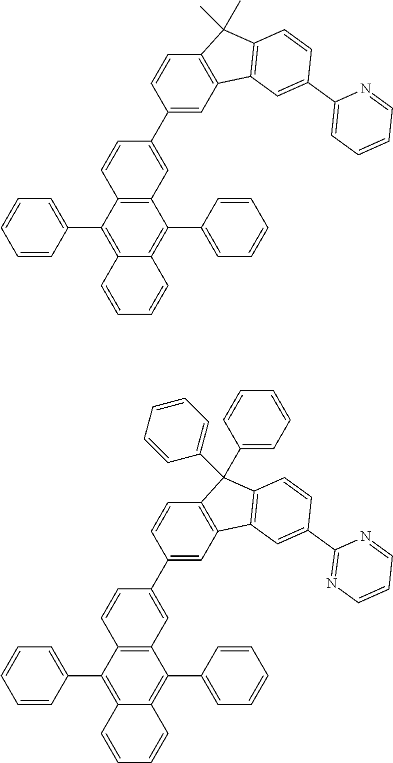 [SMILES: CC1(C)c2ccc(-c3ccc4c(-c5ccccc5)c5ccccc5c(-c5ccccc5)c4c3)cc2-c2cc(-c3ccccn3)ccc21 ; c1ccc(-c2c3ccccc3c(-c3ccccc3)c3cc(-c4ccc5c(c4)-c4cc(-c6ncccn6)ccc4C5(c4ccccc4)c4ccccc4)ccc23)cc1]

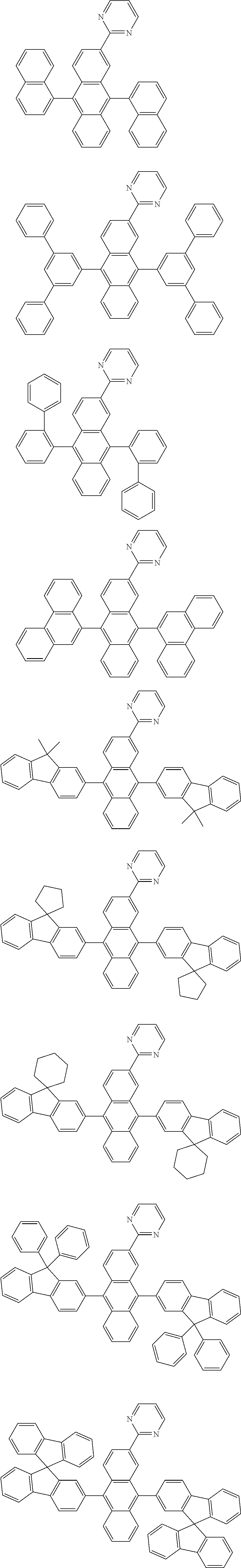

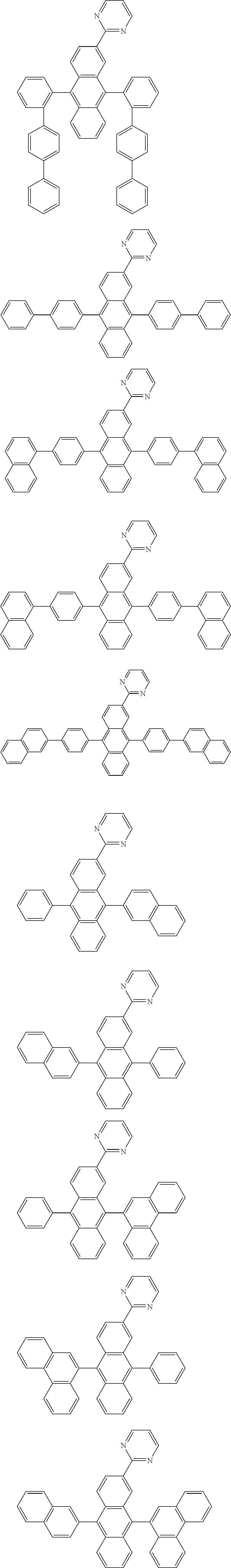

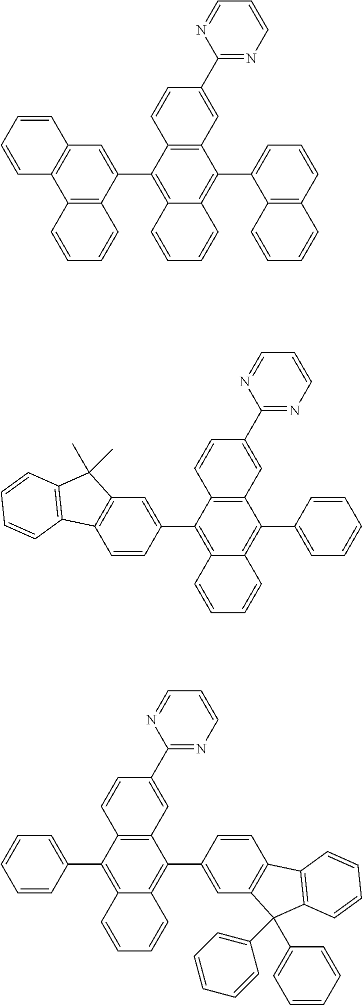 [SMILES: c1cnc(-c2ccc3c(-c4cc5ccccc5c5ccccc45)c4ccccc4c(-c4cccc5ccccc45)c3c2)nc1 ; CC1(C)c2ccccc2-c2ccc(-c3c4ccccc4c(-c4ccccc4)c4cc(-c5ncccn5)ccc34)cc21 ; c1ccc(-c2c3ccccc3c(-c3ccc4c(c3)C(c3ccccc3)(c3ccccc3)c3ccccc3-4)c3cc(-c4ncccn4)ccc23)cc1]

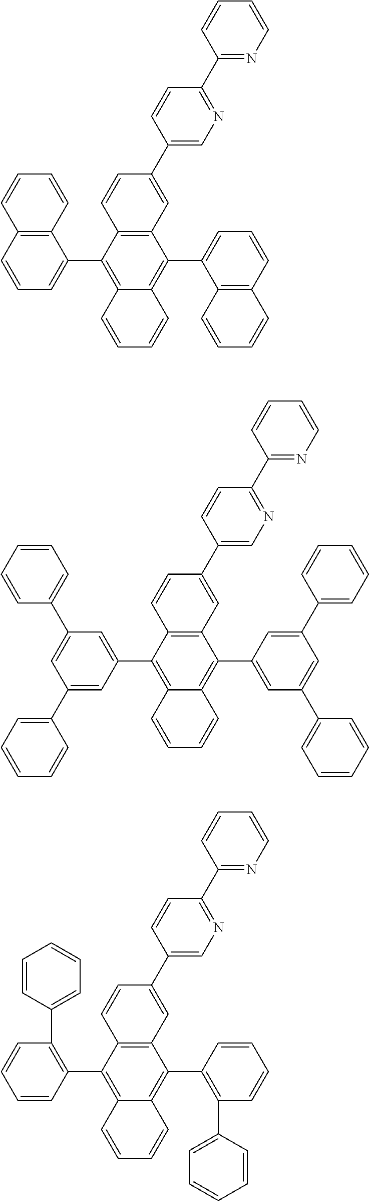 [SMILES: c1ccc(-c2ccc(-c3ccc4c(-c5cccc6ccccc56)c5ccccc5c(-c5cccc6ccccc56)c4c3)cn2)nc1 ; c1ccc(-c2cc(-c3ccccc3)cc(-c3c4ccccc4c(-c4cc(-c5ccccc5)cc(-c5ccccc5)c4)c4cc(-c5ccc(-c6ccccn6)nc5)ccc34)c2)cc1 ; c1ccc(-c2ccccc2-c2c3ccccc3c(-c3ccccc3-c3ccccc3)c3cc(-c4ccc(-c5ccccn5)nc4)ccc23)cc1]

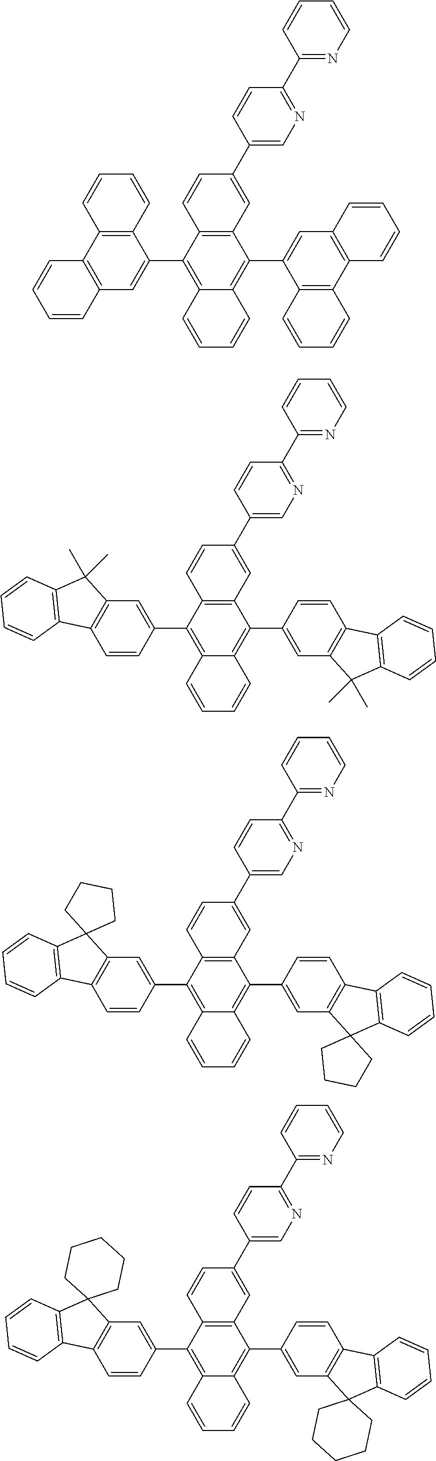

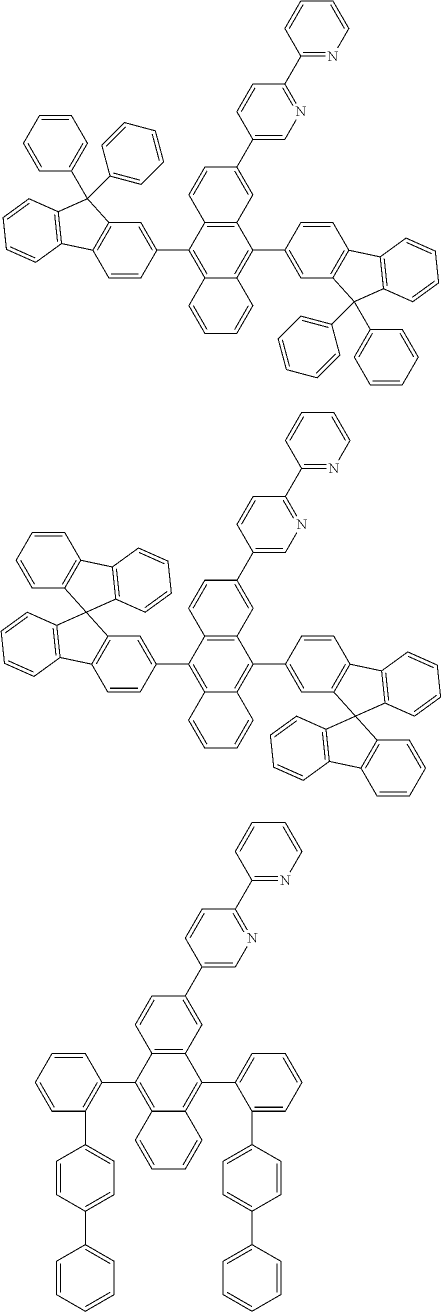

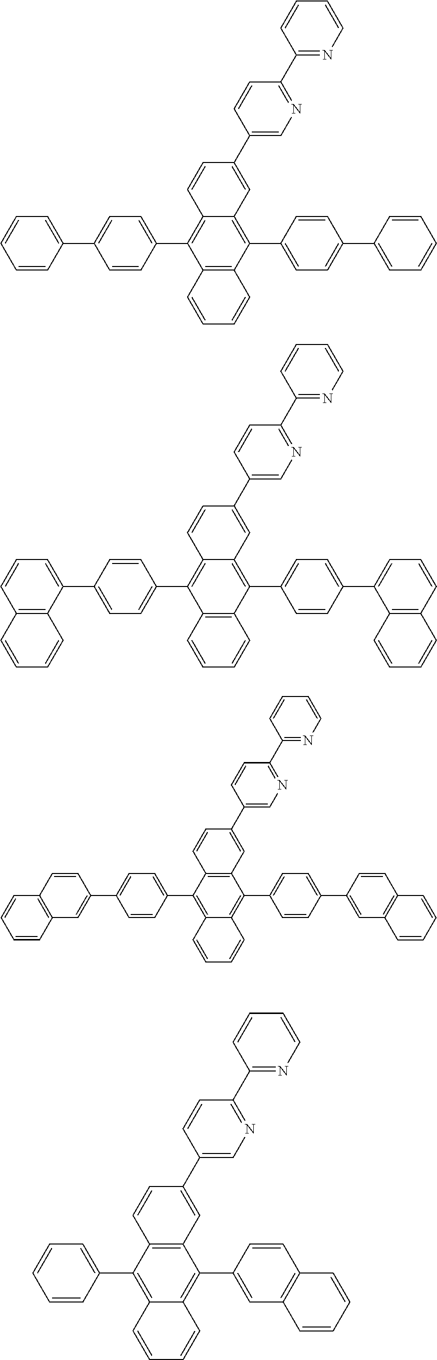 [SMILES: c1ccc(-c2ccc(-c3c4ccccc4c(-c4ccc(-c5ccccc5)cc4)c4cc(-c5ccc(-c6ccccn6)nc5)ccc34)cc2)cc1 ; c1ccc(-c2ccc(-c3ccc4c(-c5ccc(-c6cccc7ccccc67)cc5)c5ccccc5c(-c5ccc(-c6cccc7ccccc67)cc5)c4c3)cn2)nc1 ; c1ccc(-c2ccc(-c3ccc4c(-c5ccc(-c6ccc7ccccc7c6)cc5)c5ccccc5c(-c5ccc(-c6ccc7ccccc7c6)cc5)c4c3)cn2)nc1 ; c1ccc(-c2c3ccccc3c(-c3ccc4ccccc4c3)c3cc(-c4ccc(-c5ccccn5)nc4)ccc23)cc1]

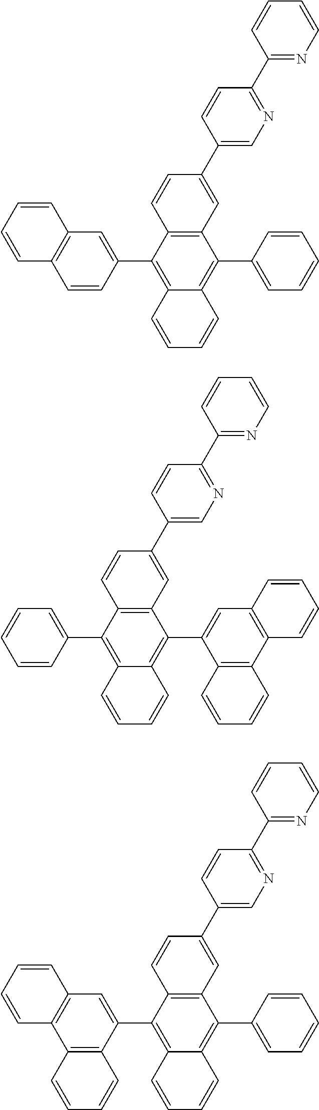

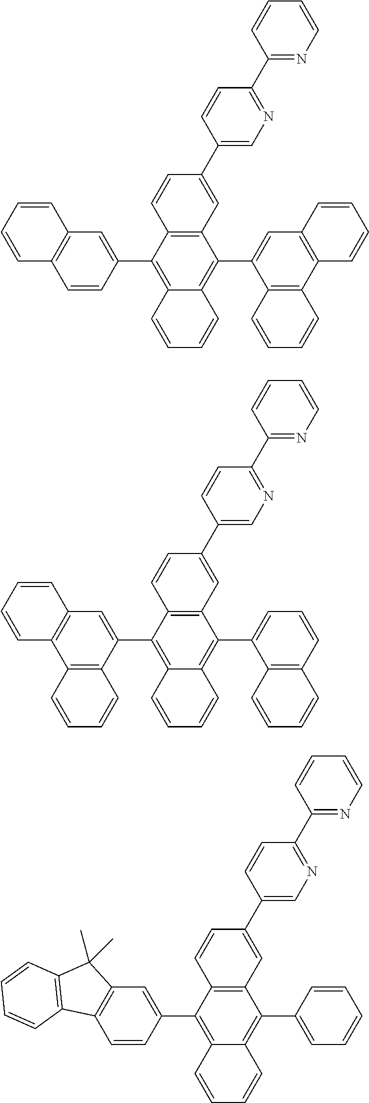

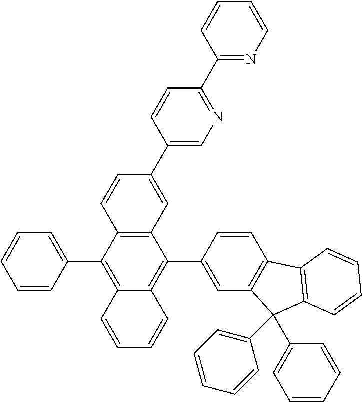 [SMILES: c1ccc(-c2c3ccccc3c(-c3ccc4c(c3)C(c3ccccc3)(c3ccccc3)c3ccccc3-4)c3cc(-c4ccc(-c5ccccn5)nc4)ccc23)cc1]

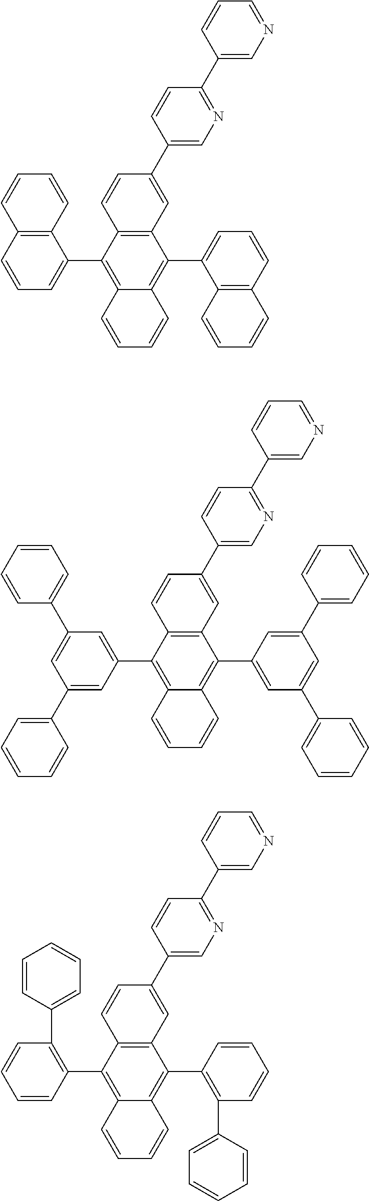 [SMILES: c1cncc(-c2ccc(-c3ccc4c(-c5cccc6ccccc56)c5ccccc5c(-c5cccc6ccccc56)c4c3)cn2)c1 ; c1ccc(-c2cc(-c3ccccc3)cc(-c3c4ccccc4c(-c4cc(-c5ccccc5)cc(-c5ccccc5)c4)c4cc(-c5ccc(-c6cccnc6)nc5)ccc34)c2)cc1 ; c1ccc(-c2ccccc2-c2c3ccccc3c(-c3ccccc3-c3ccccc3)c3cc(-c4ccc(-c5cccnc5)nc4)ccc23)cc1]

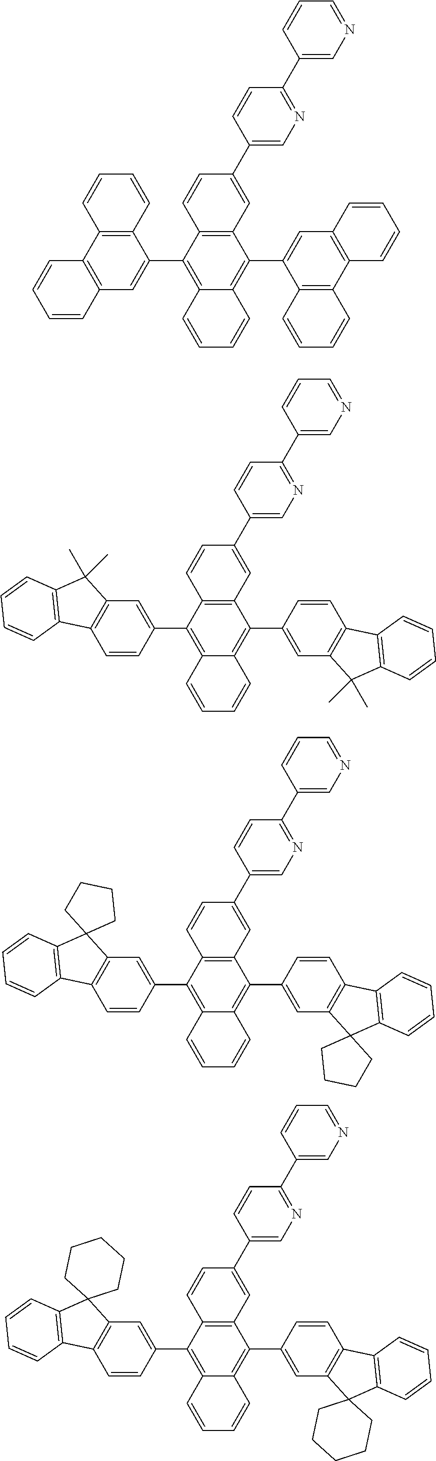

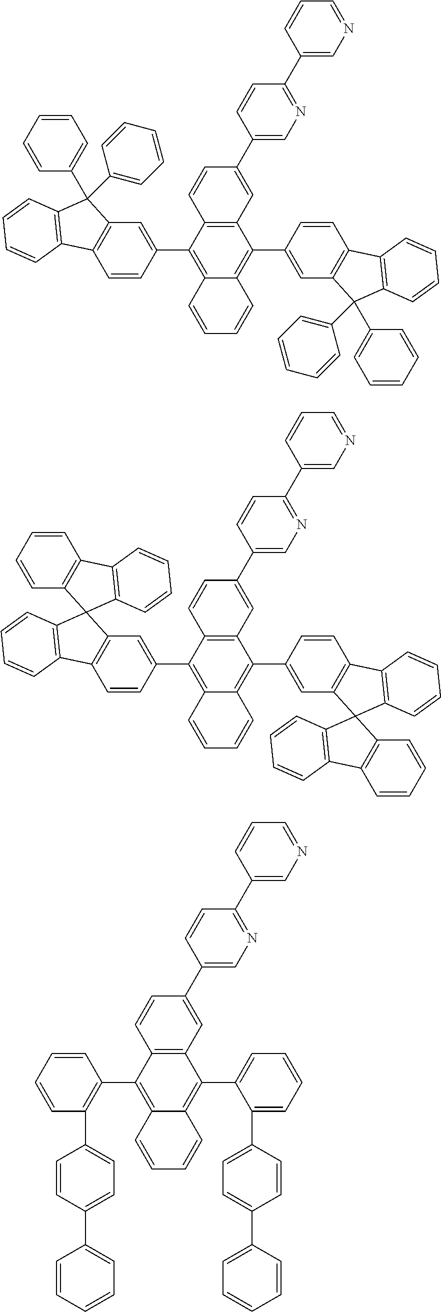

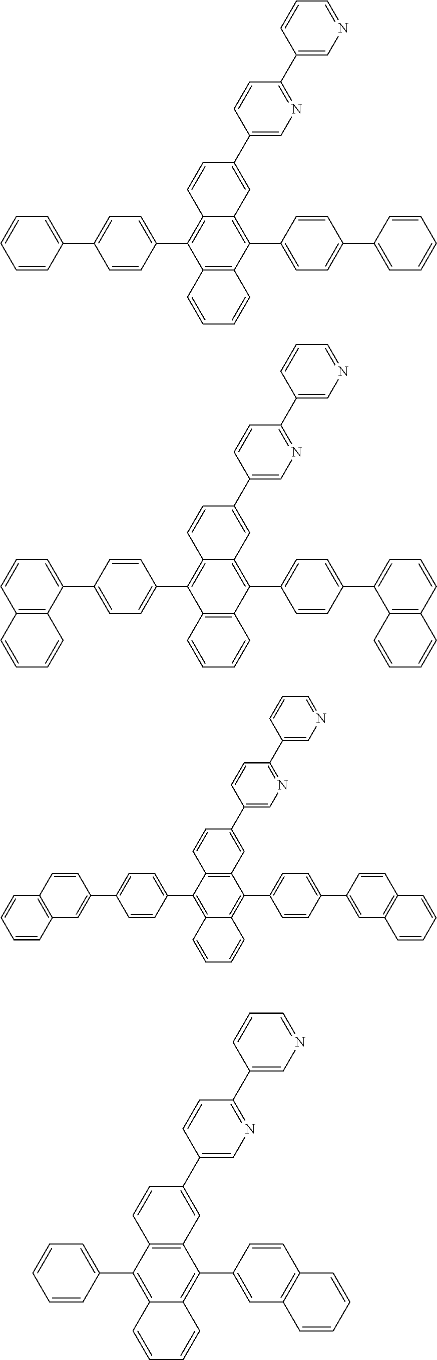 [SMILES: c1ccc(-c2ccc(-c3c4ccccc4c(-c4ccc(-c5ccccc5)cc4)c4cc(-c5ccc(-c6cccnc6)nc5)ccc34)cc2)cc1 ; c1cncc(-c2ccc(-c3ccc4c(-c5ccc(-c6cccc7ccccc67)cc5)c5ccccc5c(-c5ccc(-c6cccc7ccccc67)cc5)c4c3)cn2)c1 ; c1cncc(-c2ccc(-c3ccc4c(-c5ccc(-c6ccc7ccccc7c6)cc5)c5ccccc5c(-c5ccc(-c6ccc7ccccc7c6)cc5)c4c3)cn2)c1 ; c1ccc(-c2c3ccccc3c(-c3ccc4ccccc4c3)c3cc(-c4ccc(-c5cccnc5)nc4)ccc23)cc1]

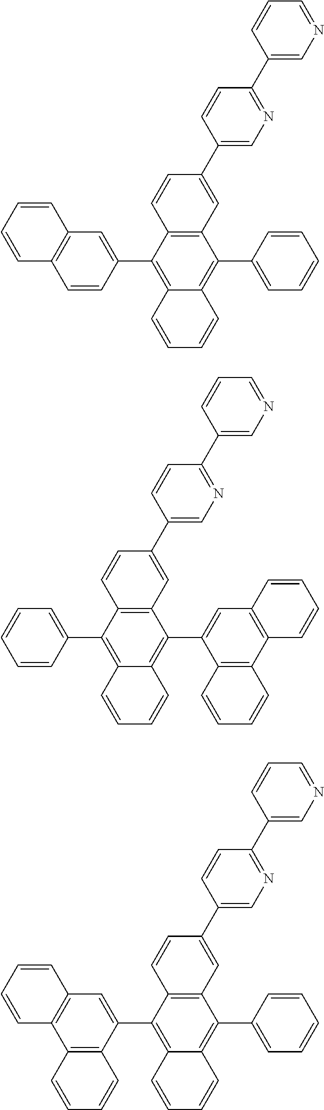

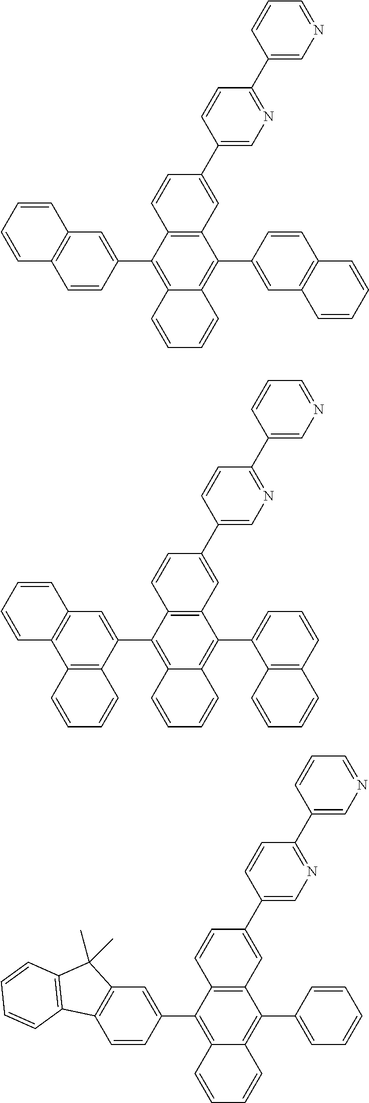

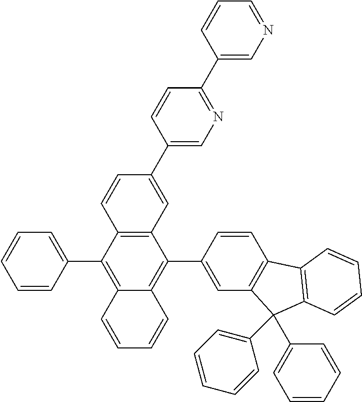 [SMILES: c1ccc(-c2c3ccccc3c(-c3ccc4c(c3)C(c3ccccc3)(c3ccccc3)c3ccccc3-4)c3cc(-c4ccc(-c5cccnc5)nc4)ccc23)cc1]

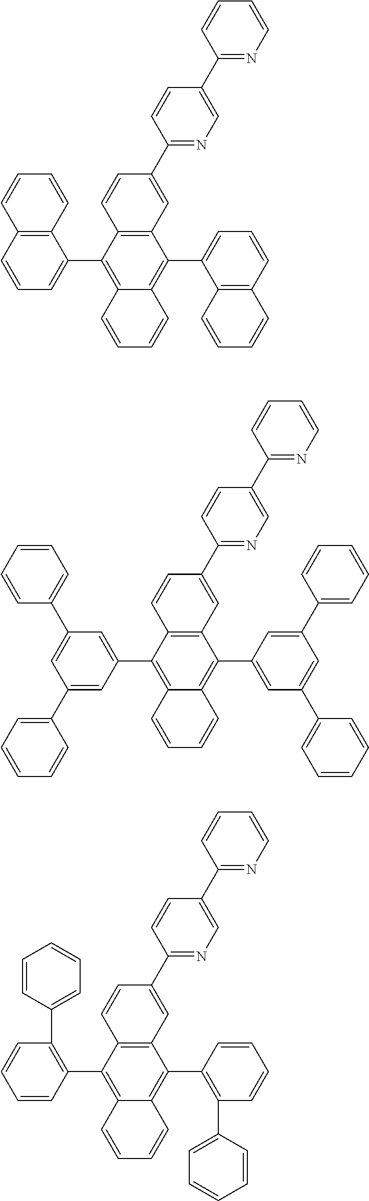 [SMILES: c1ccc(-c2ccc(-c3ccc4c(-c5cccc6ccccc56)c5ccccc5c(-c5cccc6ccccc56)c4c3)nc2)nc1 ; c1ccc(-c2cc(-c3ccccc3)cc(-c3c4ccccc4c(-c4cc(-c5ccccc5)cc(-c5ccccc5)c4)c4cc(-c5ccc(-c6ccccn6)cn5)ccc34)c2)cc1 ; c1ccc(-c2ccccc2-c2c3ccccc3c(-c3ccccc3-c3ccccc3)c3cc(-c4ccc(-c5ccccn5)cn4)ccc23)cc1]

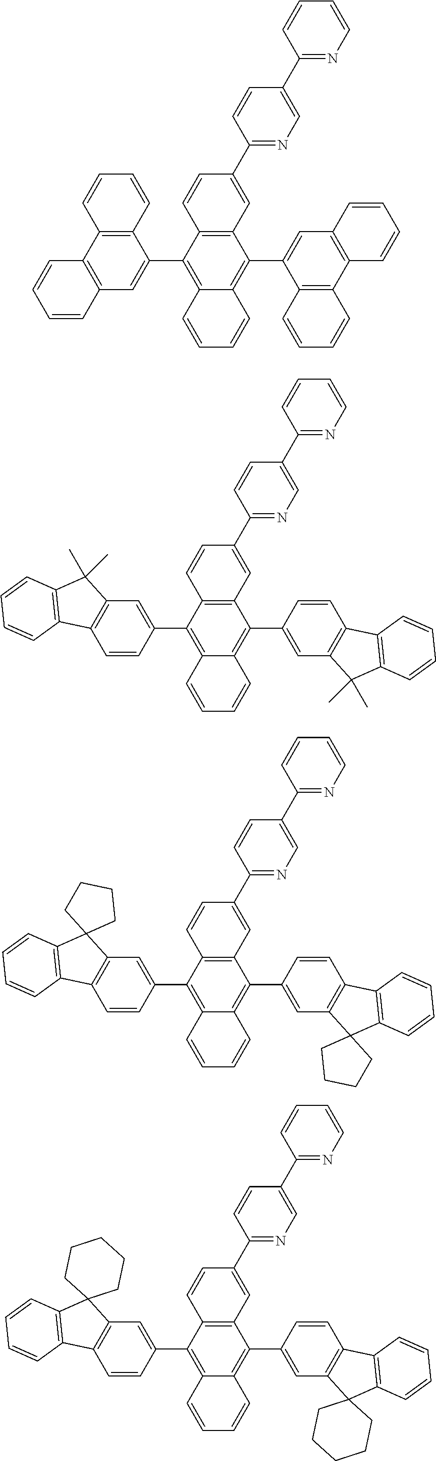

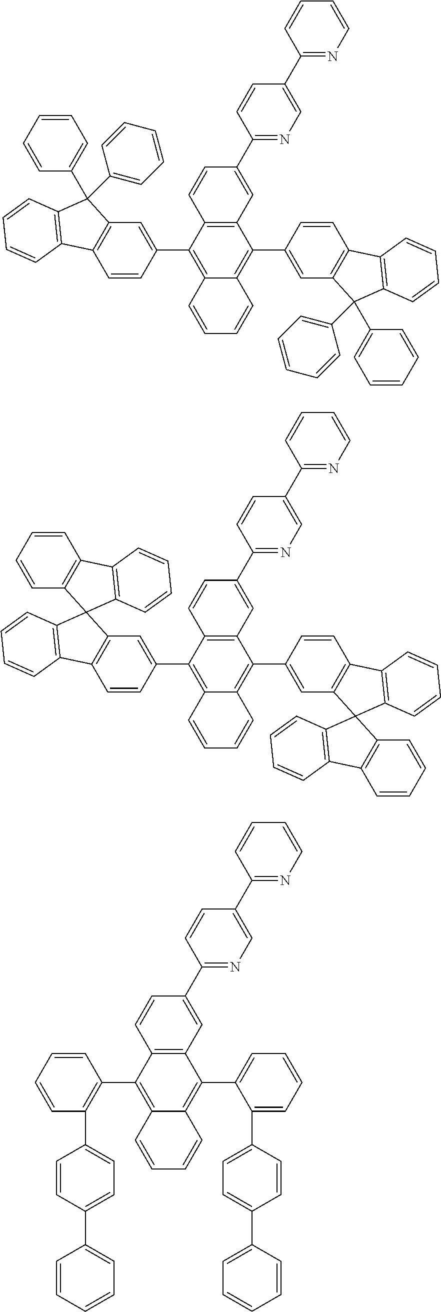

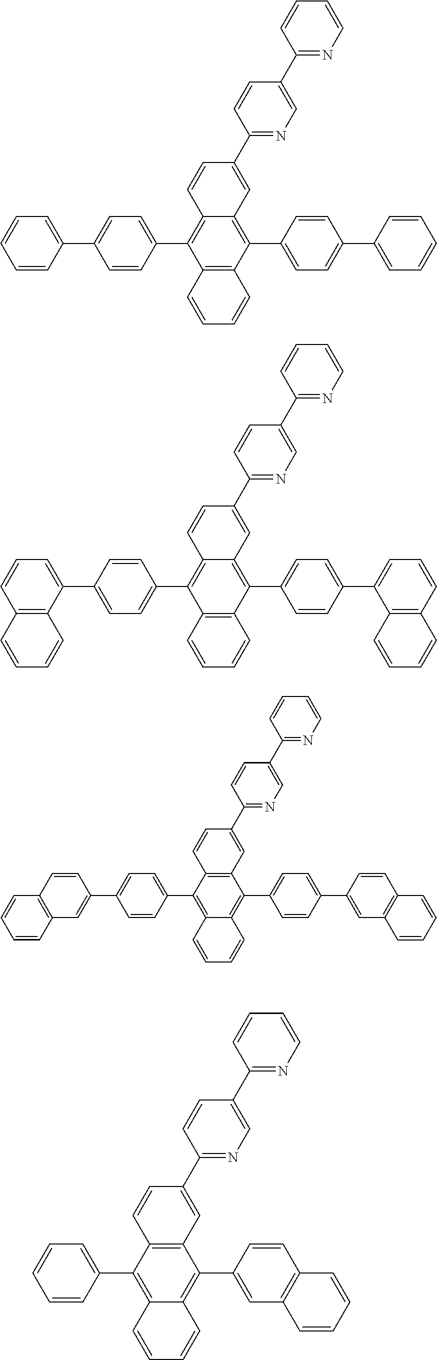 [SMILES: c1ccc(-c2ccc(-c3c4ccccc4c(-c4ccc(-c5ccccc5)cc4)c4cc(-c5ccc(-c6ccccn6)cn5)ccc34)cc2)cc1 ; c1ccc(-c2ccc(-c3ccc4c(-c5ccc(-c6cccc7ccccc67)cc5)c5ccccc5c(-c5ccc(-c6cccc7ccccc67)cc5)c4c3)nc2)nc1 ; c1ccc(-c2ccc(-c3ccc4c(-c5ccc(-c6ccc7ccccc7c6)cc5)c5ccccc5c(-c5ccc(-c6ccc7ccccc7c6)cc5)c4c3)nc2)nc1 ; c1ccc(-c2c3ccccc3c(-c3ccc4ccccc4c3)c3cc(-c4ccc(-c5ccccn5)cn4)ccc23)cc1]

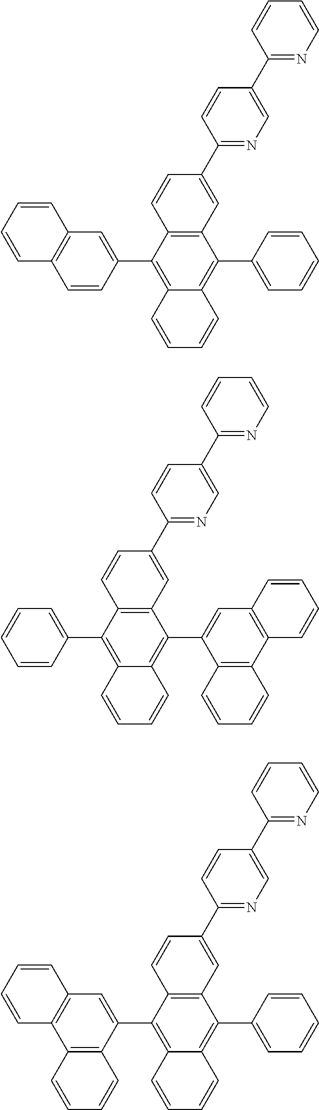

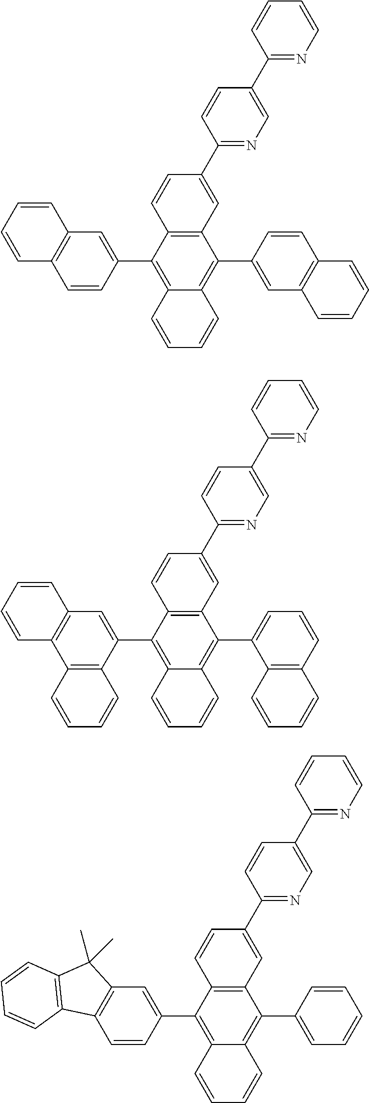

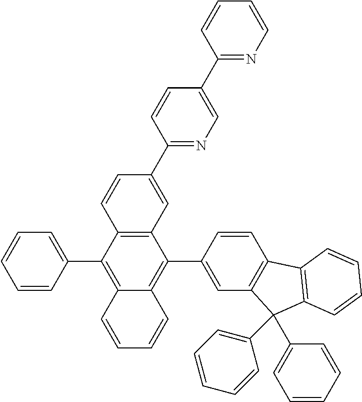 [SMILES: c1ccc(-c2c3ccccc3c(-c3ccc4c(c3)C(c3ccccc3)(c3ccccc3)c3ccccc3-4)c3cc(-c4ccc(-c5ccccn5)cn4)ccc23)cc1]

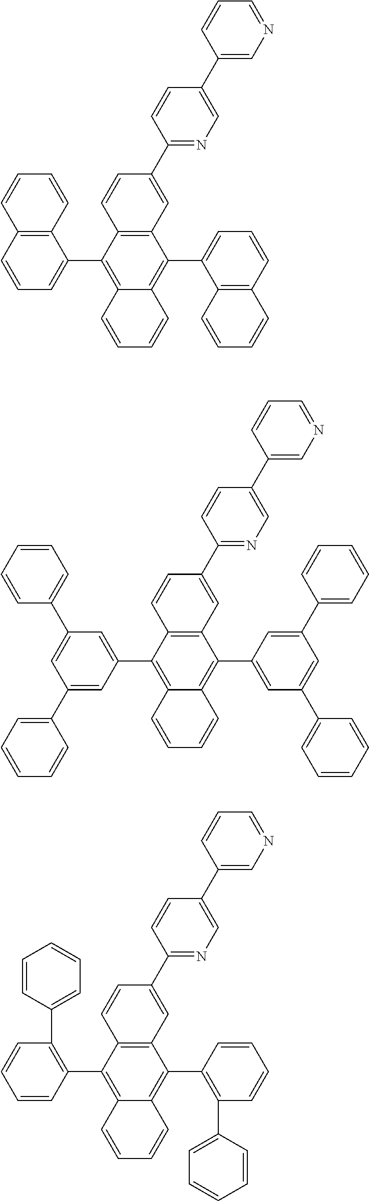 [SMILES: c1cncc(-c2ccc(-c3ccc4c(-c5cccc6ccccc56)c5ccccc5c(-c5cccc6ccccc56)c4c3)nc2)c1 ; c1ccc(-c2cc(-c3ccccc3)cc(-c3c4ccccc4c(-c4cc(-c5ccccc5)cc(-c5ccccc5)c4)c4cc(-c5ccc(-c6cccnc6)cn5)ccc34)c2)cc1 ; c1ccc(-c2ccccc2-c2c3ccccc3c(-c3ccccc3-c3ccccc3)c3cc(-c4ccc(-c5cccnc5)cn4)ccc23)cc1]

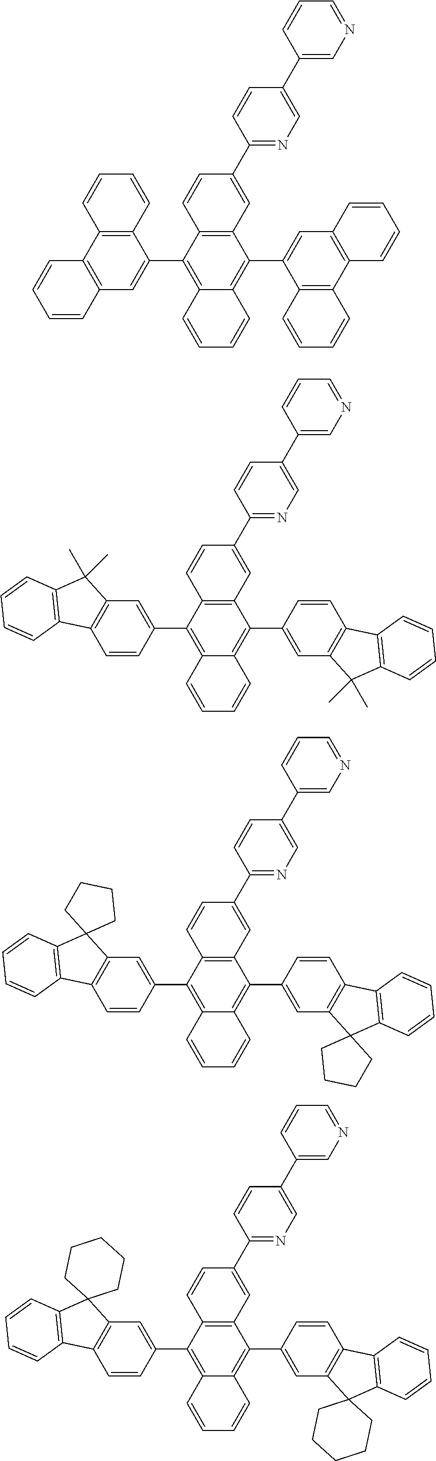

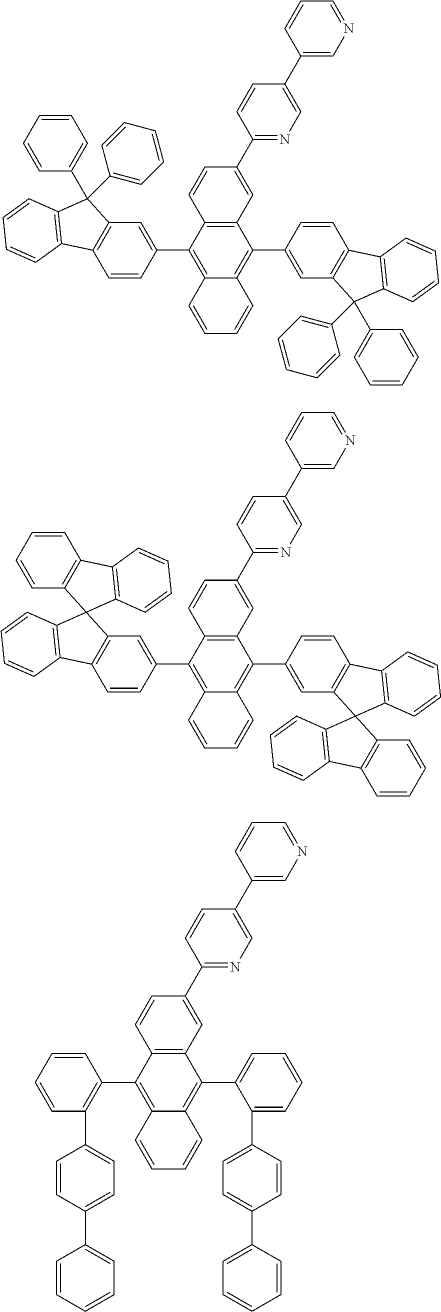

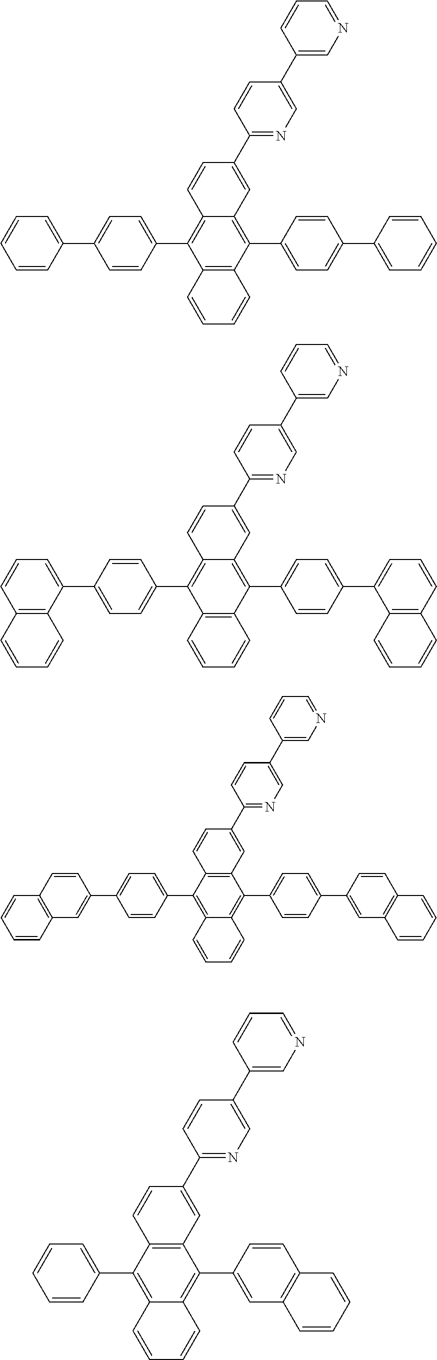 [SMILES: c1ccc(-c2ccc(-c3c4ccccc4c(-c4ccc(-c5ccccc5)cc4)c4cc(-c5ccc(-c6cccnc6)cn5)ccc34)cc2)cc1 ; c1cncc(-c2ccc(-c3ccc4c(-c5ccc(-c6cccc7ccccc67)cc5)c5ccccc5c(-c5ccc(-c6cccc7ccccc67)cc5)c4c3)nc2)c1 ; c1cncc(-c2ccc(-c3ccc4c(-c5ccc(-c6ccc7ccccc7c6)cc5)c5ccccc5c(-c5ccc(-c6ccc7ccccc7c6)cc5)c4c3)nc2)c1 ; c1ccc(-c2c3ccccc3c(-c3ccc4ccccc4c3)c3cc(-c4ccc(-c5cccnc5)cn4)ccc23)cc1]

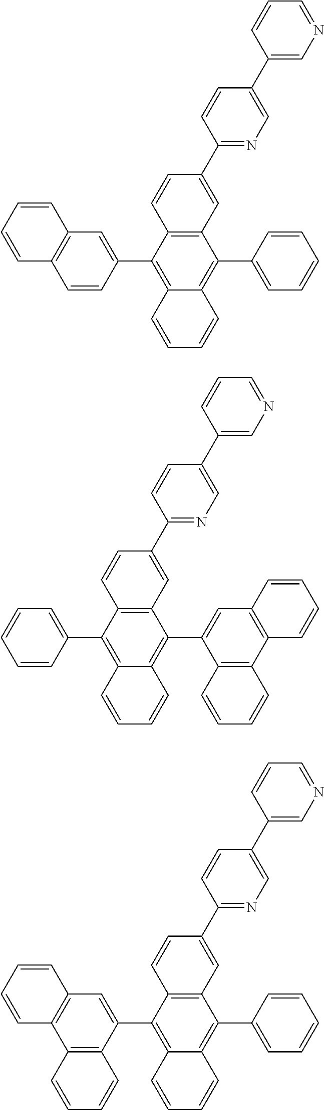

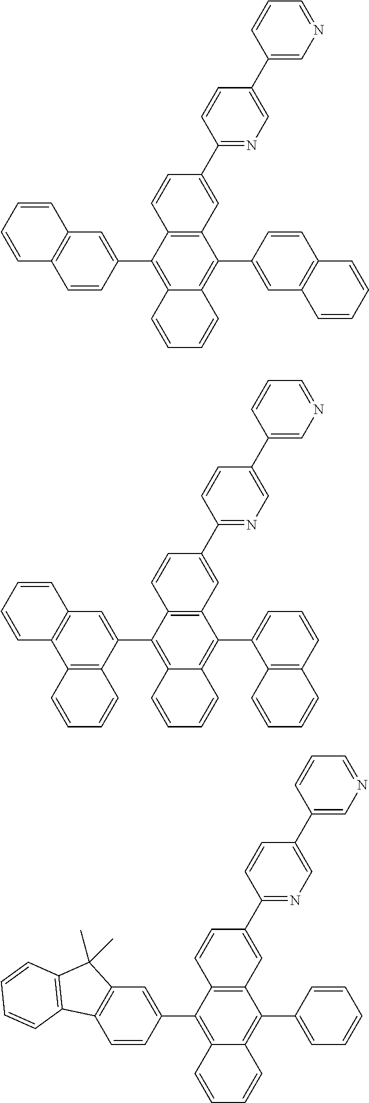

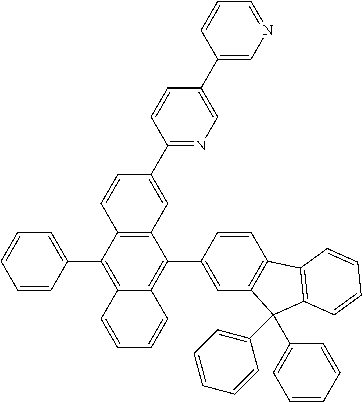 [SMILES: c1ccc(-c2c3ccccc3c(-c3ccc4c(c3)C(c3ccccc3)(c3ccccc3)c3ccccc3-4)c3cc(-c4ccc(-c5cccnc5)cn4)ccc23)cc1]

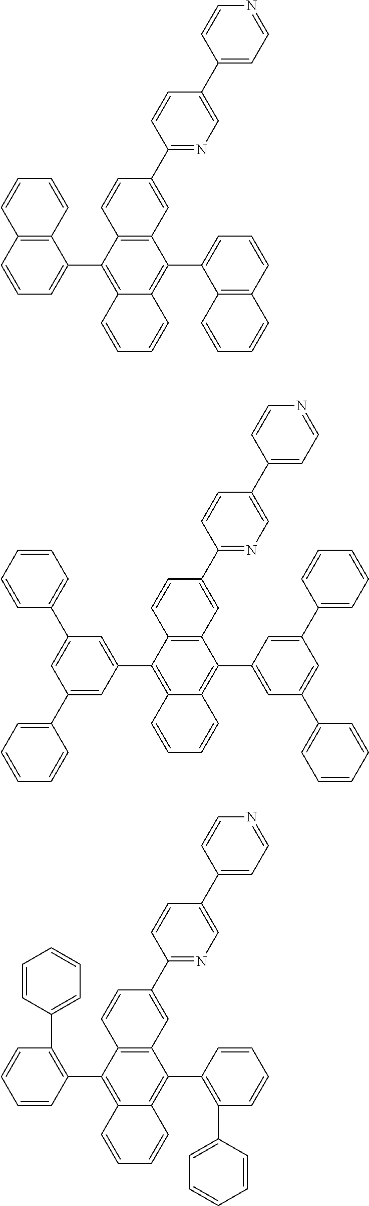 [SMILES: c1ccc2c(-c3c4ccccc4c(-c4cccc5ccccc45)c4cc(-c5ccc(-c6ccncc6)cn5)ccc34)cccc2c1 ; c1ccc(-c2cc(-c3ccccc3)cc(-c3c4ccccc4c(-c4cc(-c5ccccc5)cc(-c5ccccc5)c4)c4cc(-c5ccc(-c6ccncc6)cn5)ccc34)c2)cc1 ; c1ccc(-c2ccccc2-c2c3ccccc3c(-c3ccccc3-c3ccccc3)c3cc(-c4ccc(-c5ccncc5)cn4)ccc23)cc1]

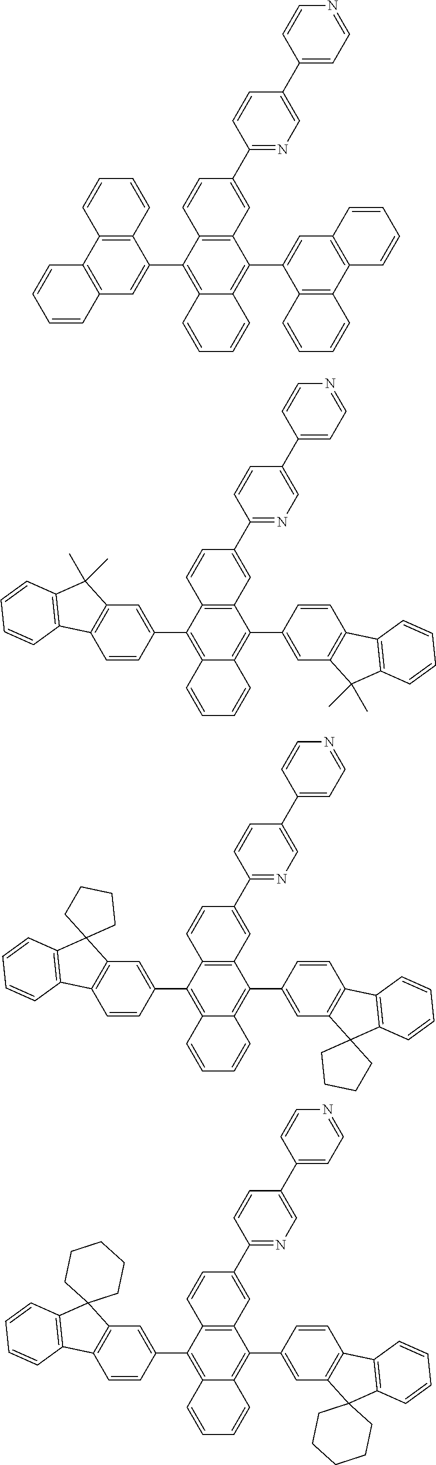

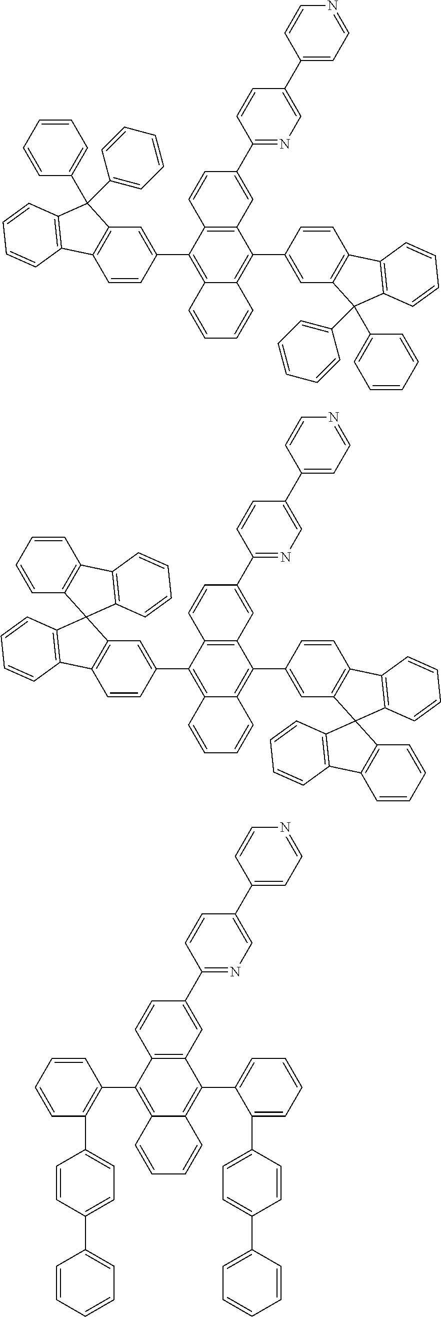

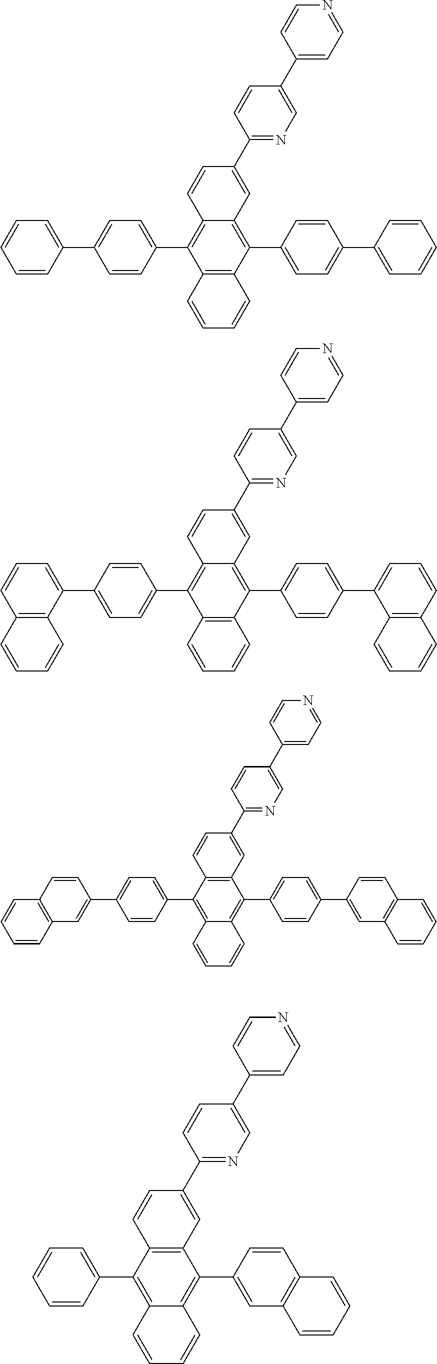 [SMILES: c1ccc(-c2ccc(-c3c4ccccc4c(-c4ccc(-c5ccccc5)cc4)c4cc(-c5ccc(-c6ccncc6)cn5)ccc34)cc2)cc1 ; c1ccc2c(-c3ccc(-c4c5ccccc5c(-c5ccc(-c6cccc7ccccc67)cc5)c5cc(-c6ccc(-c7ccncc7)cn6)ccc45)cc3)cccc2c1 ; c1ccc2cc(-c3ccc(-c4c5ccccc5c(-c5ccc(-c6ccc7ccccc7c6)cc5)c5cc(-c6ccc(-c7ccncc7)cn6)ccc45)cc3)ccc2c1 ; c1ccc(-c2c3ccccc3c(-c3ccc4ccccc4c3)c3cc(-c4ccc(-c5ccncc5)cn4)ccc23)cc1]

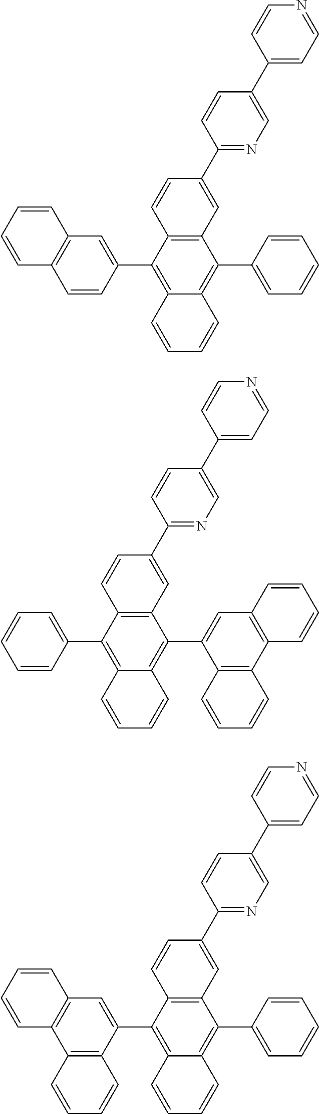

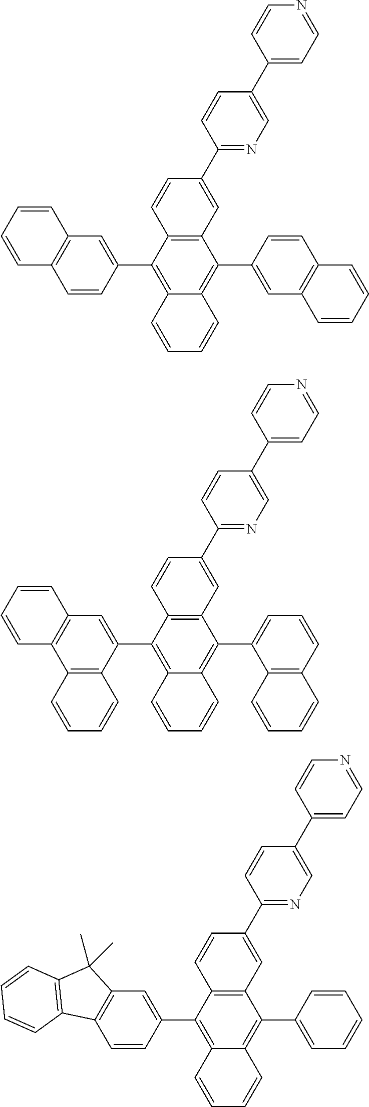

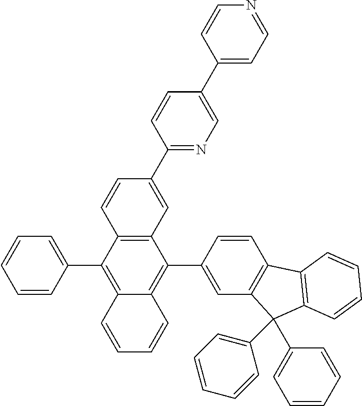 [SMILES: c1ccc(-c2c3ccccc3c(-c3ccc4c(c3)C(c3ccccc3)(c3ccccc3)c3ccccc3-4)c3cc(-c4ccc(-c5ccncc5)cn4)ccc23)cc1]

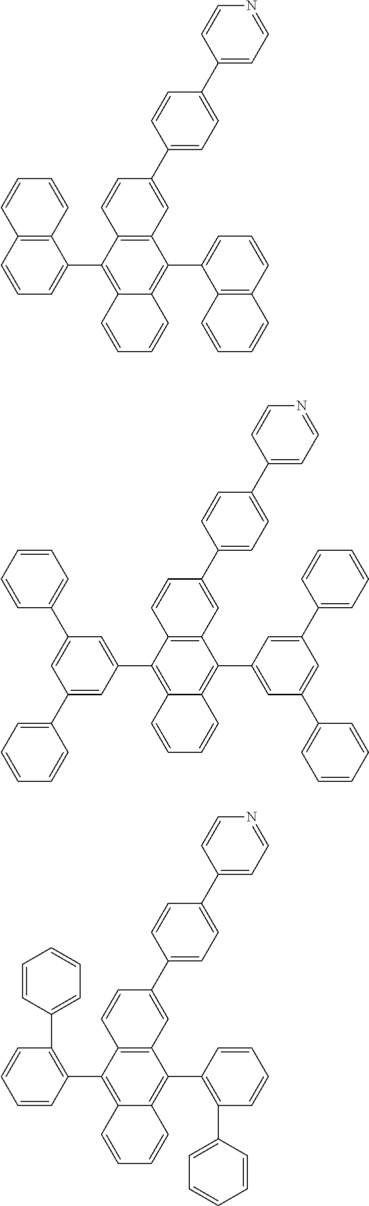 [SMILES: c1ccc2c(-c3c4ccccc4c(-c4cccc5ccccc45)c4cc(-c5ccc(-c6ccncc6)cc5)ccc34)cccc2c1 ; c1ccc(-c2cc(-c3ccccc3)cc(-c3c4ccccc4c(-c4cc(-c5ccccc5)cc(-c5ccccc5)c4)c4cc(-c5ccc(-c6ccncc6)cc5)ccc34)c2)cc1 ; c1ccc(-c2ccccc2-c2c3ccccc3c(-c3ccccc3-c3ccccc3)c3cc(-c4ccc(-c5ccncc5)cc4)ccc23)cc1]

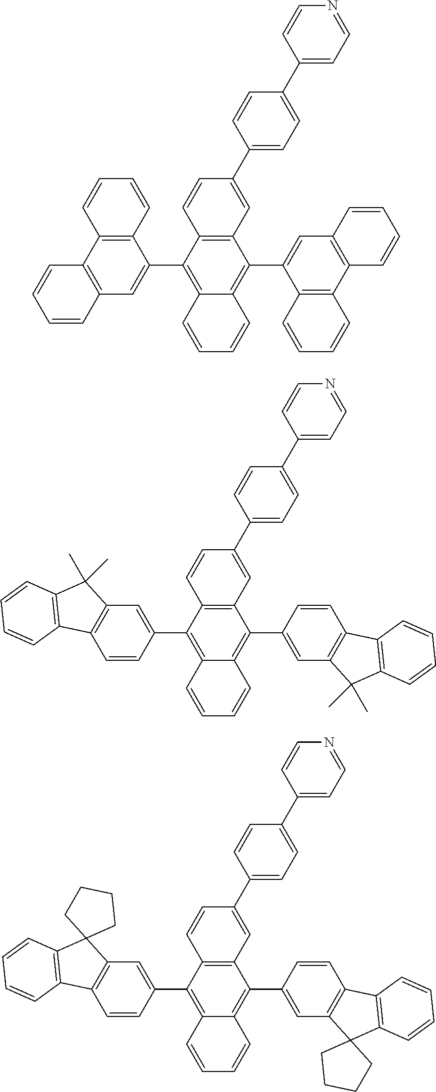

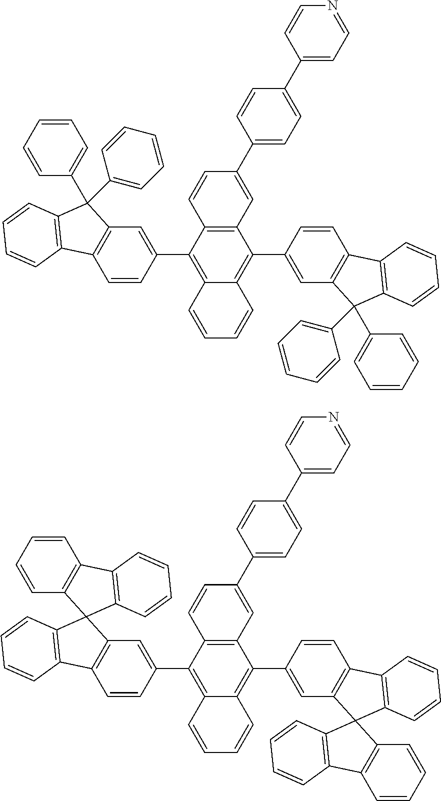

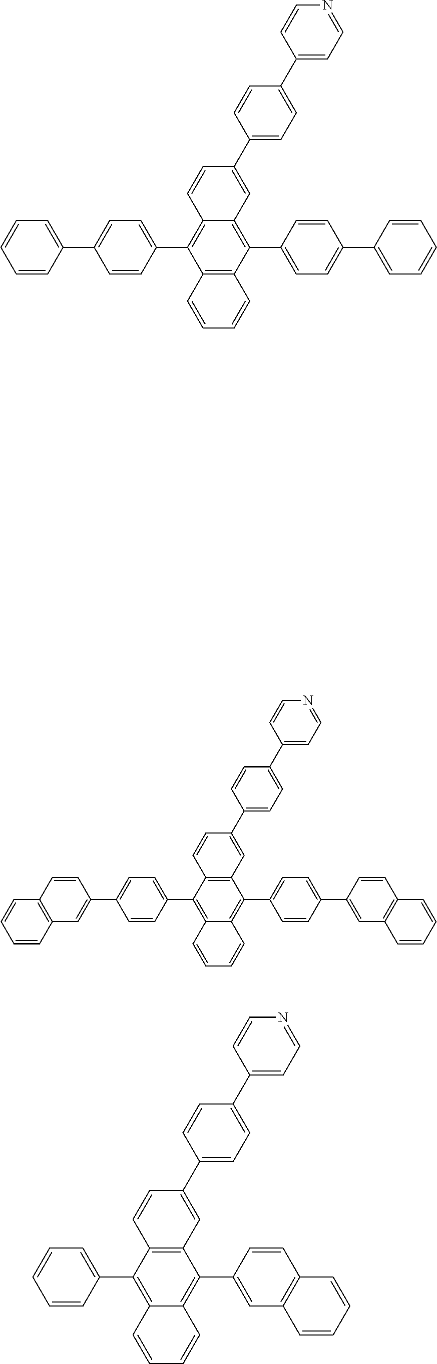 [SMILES: c1ccc(-c2ccc(-c3c4ccccc4c(-c4ccc(-c5ccccc5)cc4)c4cc(-c5ccc(-c6ccncc6)cc5)ccc34)cc2)cc1 ; c1ccc2cc(-c3ccc(-c4c5ccccc5c(-c5ccc(-c6ccc7ccccc7c6)cc5)c5cc(-c6ccc(-c7ccncc7)cc6)ccc45)cc3)ccc2c1 ; c1ccc(-c2c3ccccc3c(-c3ccc4ccccc4c3)c3cc(-c4ccc(-c5ccncc5)cc4)ccc23)cc1]

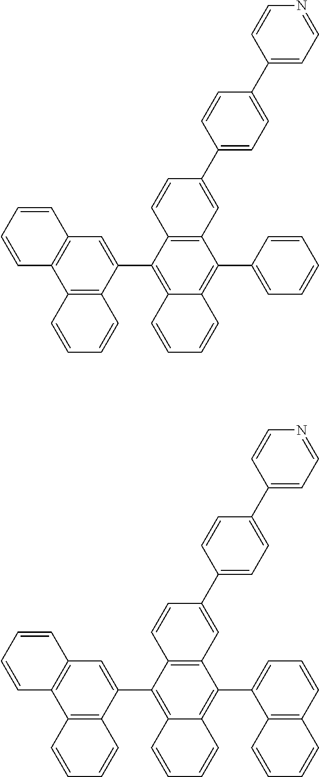 [SMILES: c1ccc(-c2c3ccccc3c(-c3cc4ccccc4c4ccccc34)c3ccc(-c4ccc(-c5ccncc5)cc4)cc23)cc1 ; c1ccc2c(-c3c4ccccc4c(-c4cc5ccccc5c5ccccc45)c4ccc(-c5ccc(-c6ccncc6)cc5)cc34)cccc2c1]

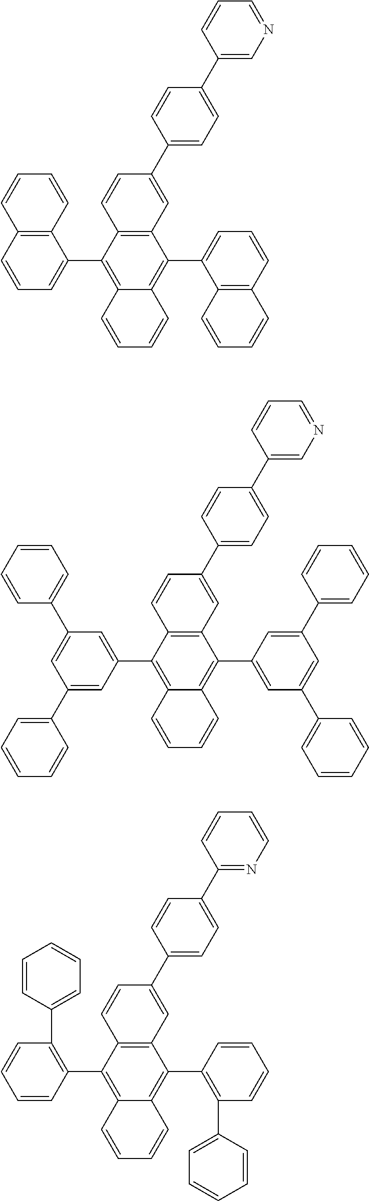 [SMILES: c1cncc(-c2ccc(-c3ccc4c(-c5cccc6ccccc56)c5ccccc5c(-c5cccc6ccccc56)c4c3)cc2)c1 ; c1ccc(-c2cc(-c3ccccc3)cc(-c3c4ccccc4c(-c4cc(-c5ccccc5)cc(-c5ccccc5)c4)c4cc(-c5ccc(-c6cccnc6)cc5)ccc34)c2)cc1 ; c1ccc(-c2ccccc2-c2c3ccccc3c(-c3ccccc3-c3ccccc3)c3cc(-c4ccc(-c5ccccn5)cc4)ccc23)cc1]

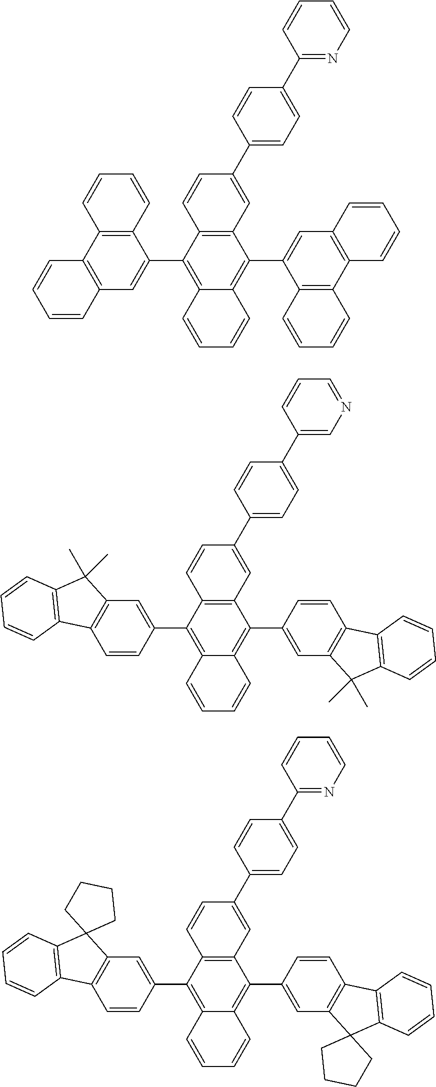

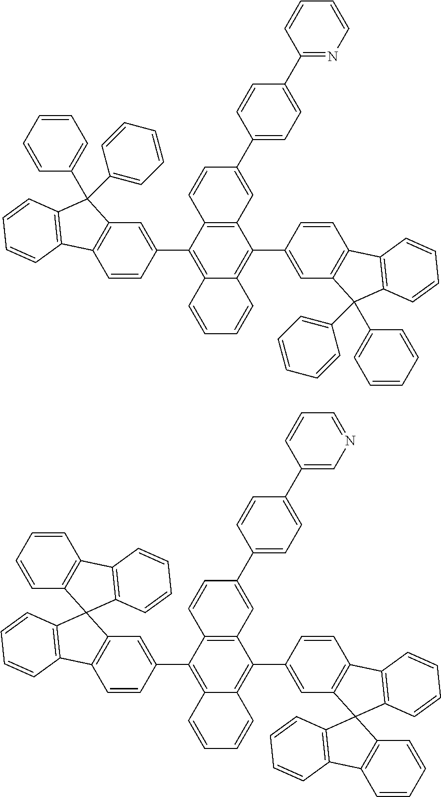

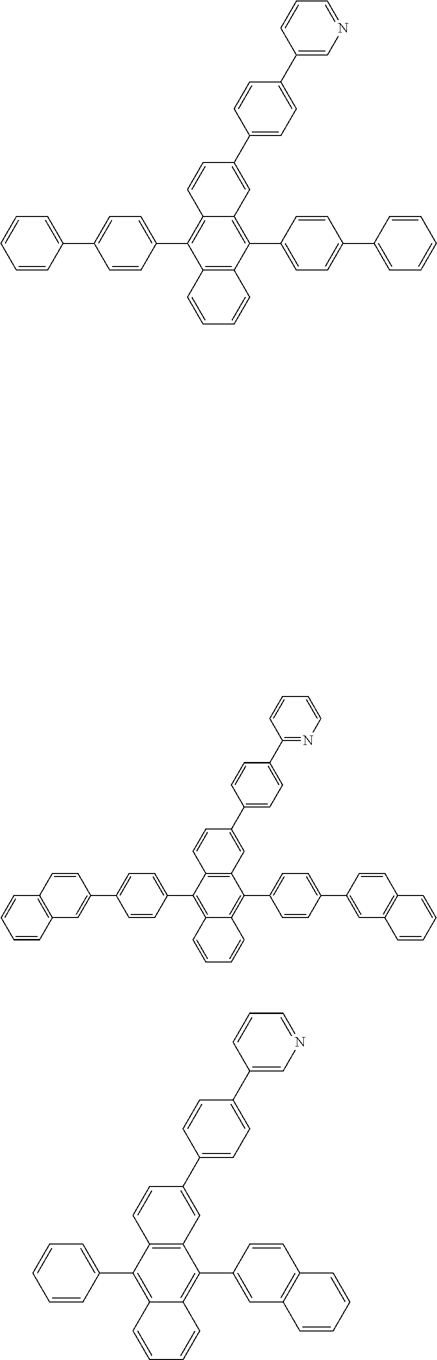 [SMILES: c1ccc(-c2ccc(-c3c4ccccc4c(-c4ccc(-c5ccccc5)cc4)c4cc(-c5ccc(-c6cccnc6)cc5)ccc34)cc2)cc1 ; c1ccc(-c2ccc(-c3ccc4c(-c5ccc(-c6ccc7ccccc7c6)cc5)c5ccccc5c(-c5ccc(-c6ccc7ccccc7c6)cc5)c4c3)cc2)nc1 ; c1ccc(-c2c3ccccc3c(-c3ccc4ccccc4c3)c3cc(-c4ccc(-c5cccnc5)cc4)ccc23)cc1]

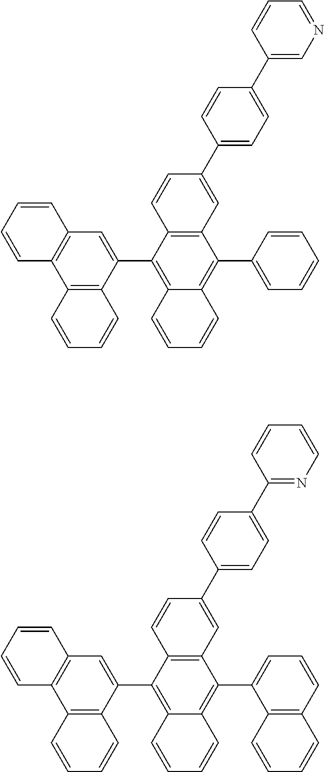 [SMILES: c1ccc(-c2c3ccccc3c(-c3cc4ccccc4c4ccccc34)c3ccc(-c4ccc(-c5cccnc5)cc4)cc23)cc1 ; c1ccc(-c2ccc(-c3ccc4c(-c5cc6ccccc6c6ccccc56)c5ccccc5c(-c5cccc6ccccc56)c4c3)cc2)nc1]

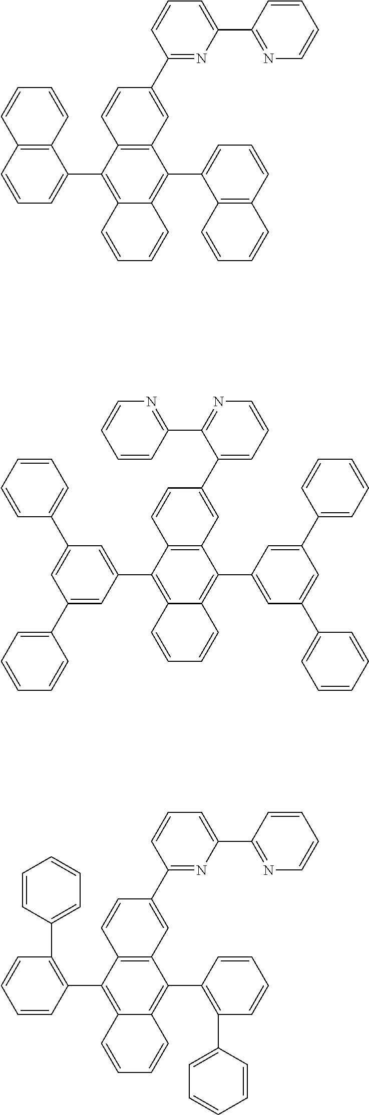 [SMILES: c1ccc(-c2cccc(-c3ccc4c(-c5cccc6ccccc56)c5ccccc5c(-c5cccc6ccccc56)c4c3)n2)nc1 ; c1ccc(-c2cc(-c3ccccc3)cc(-c3c4ccccc4c(-c4cc(-c5ccccc5)cc(-c5ccccc5)c4)c4cc(-c5cccnc5-c5ccccn5)ccc34)c2)cc1 ; c1ccc(-c2ccccc2-c2c3ccccc3c(-c3ccccc3-c3ccccc3)c3cc(-c4cccc(-c5ccccn5)n4)ccc23)cc1]

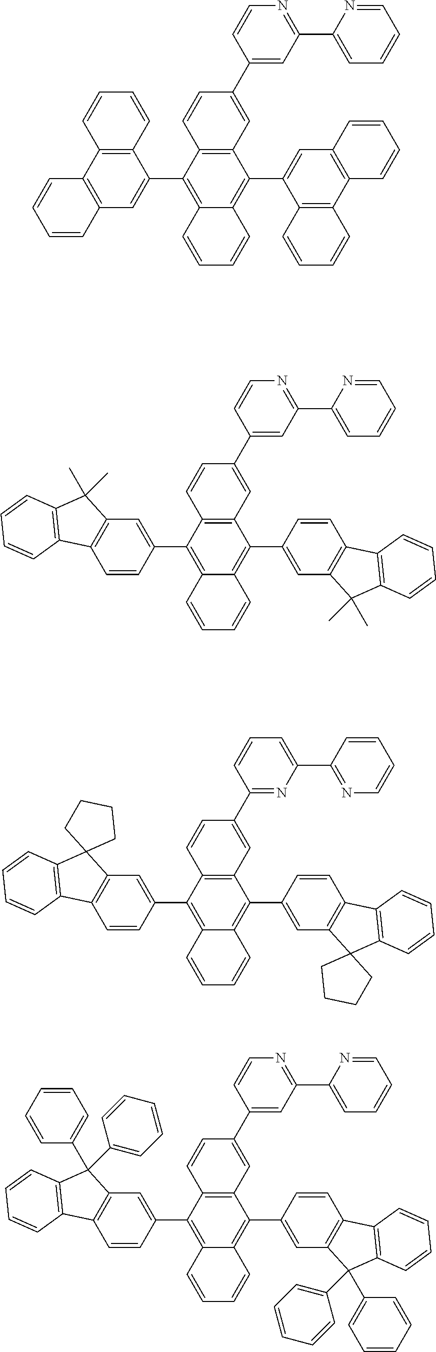 [SMILES: c1ccc(-c2cc(-c3ccc4c(-c5cc6ccccc6c6ccccc56)c5ccccc5c(-c5cc6ccccc6c6ccccc56)c4c3)ccn2)nc1 ; CC1(C)c2ccccc2-c2ccc(-c3c4ccccc4c(-c4ccc5c(c4)C(C)(C)c4ccccc4-5)c4cc(-c5ccnc(-c6ccccn6)c5)ccc34)cc21 ; c1ccc(-c2cccc(-c3ccc4c(-c5ccc6c(c5)C5(CCCC5)c5ccccc5-6)c5ccccc5c(-c5ccc6c(c5)C5(CCCC5)c5ccccc5-6)c4c3)n2)nc1 ; c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccnc(-c7ccccn7)c6)ccc45)cc32)cc1]

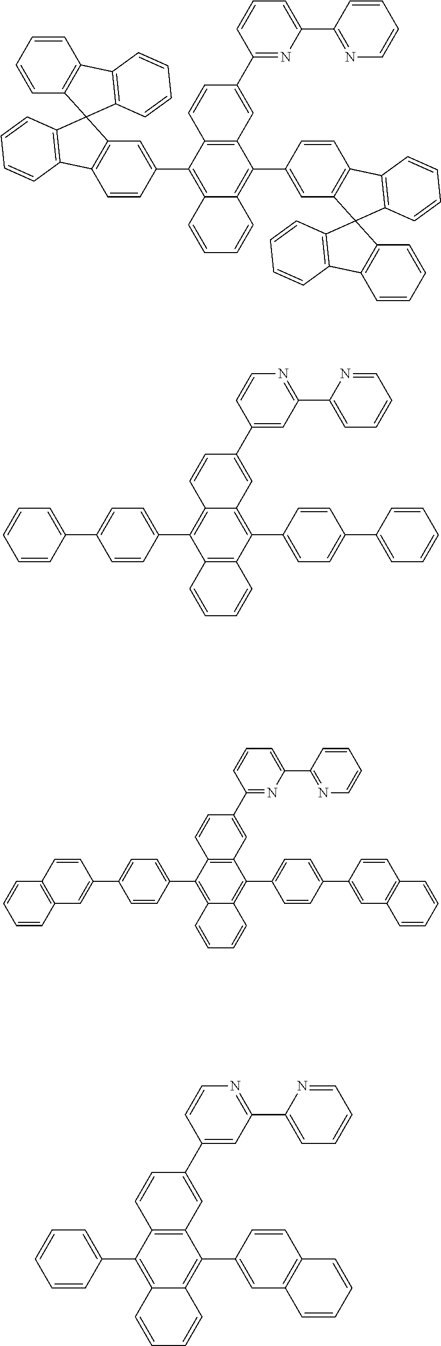 [SMILES: c1ccc(-c2cccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)n2)nc1 ; c1ccc(-c2ccc(-c3c4ccccc4c(-c4ccc(-c5ccccc5)cc4)c4cc(-c5ccnc(-c6ccccn6)c5)ccc34)cc2)cc1 ; c1ccc(-c2cccc(-c3ccc4c(-c5ccc(-c6ccc7ccccc7c6)cc5)c5ccccc5c(-c5ccc(-c6ccc7ccccc7c6)cc5)c4c3)n2)nc1 ; c1ccc(-c2c3ccccc3c(-c3ccc4ccccc4c3)c3cc(-c4ccnc(-c5ccccn5)c4)ccc23)cc1]

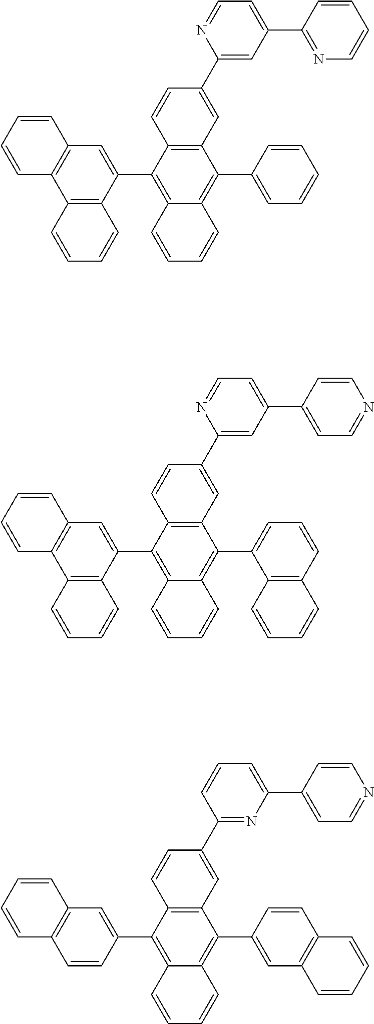 [SMILES: c1ccc(-c2c3ccccc3c(-c3cc4ccccc4c4ccccc34)c3ccc(-c4cc(-c5ccccn5)ccn4)cc23)cc1 ; c1ccc2c(-c3c4ccccc4c(-c4cc5ccccc5c5ccccc45)c4ccc(-c5cc(-c6ccncc6)ccn5)cc34)cccc2c1 ; c1cc(-c2ccncc2)nc(-c2ccc3c(-c4ccc5ccccc5c4)c4ccccc4c(-c4ccc5ccccc5c4)c3c2)c1]

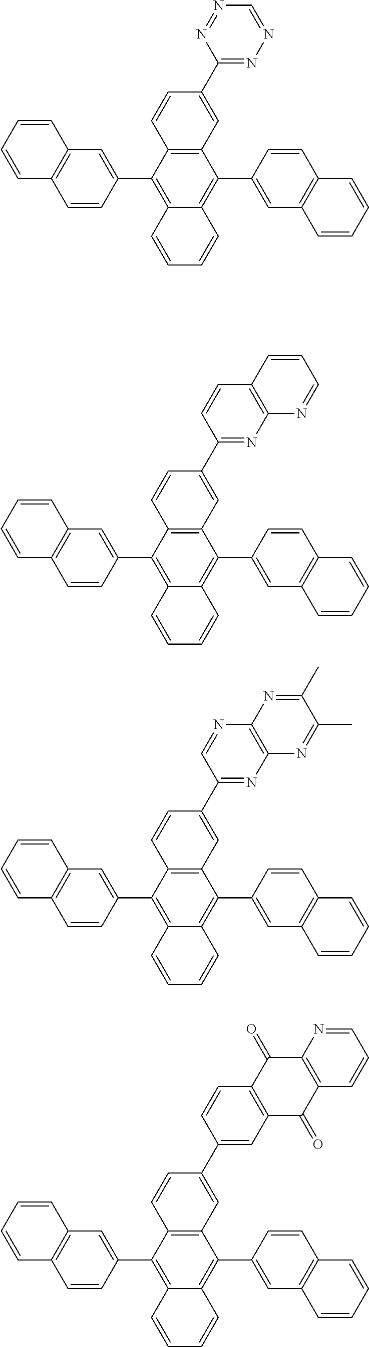 [SMILES: c1ccc2cc(-c3c4ccccc4c(-c4ccc5ccccc5c4)c4cc(-c5nncnn5)ccc34)ccc2c1 ; c1ccc2cc(-c3c4ccccc4c(-c4ccc5ccccc5c4)c4cc(-c5ccc6cccnc6n5)ccc34)ccc2c1 ; Cc1nc2ncc(-c3ccc4c(-c5ccc6ccccc6c5)c5ccccc5c(-c5ccc6ccccc6c5)c4c3)nc2nc1C ; O=C1c2cc(-c3ccc4c(-c5ccc6ccccc6c5)c5ccccc5c(-c5ccc6ccccc6c5)c4c3)ccc2C(=O)c2ncccc21]

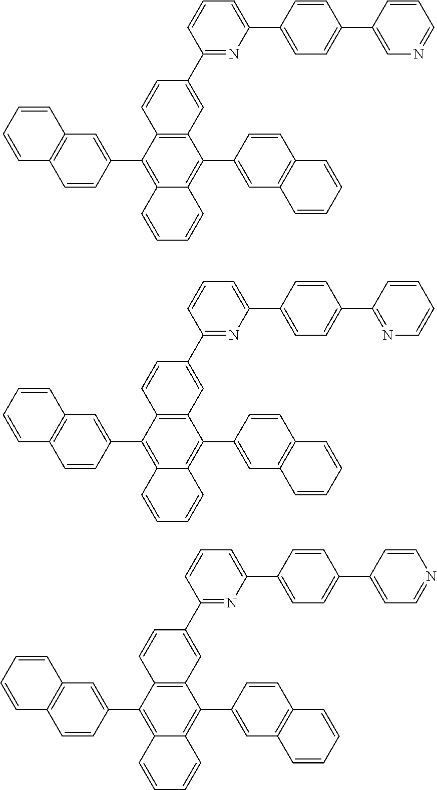

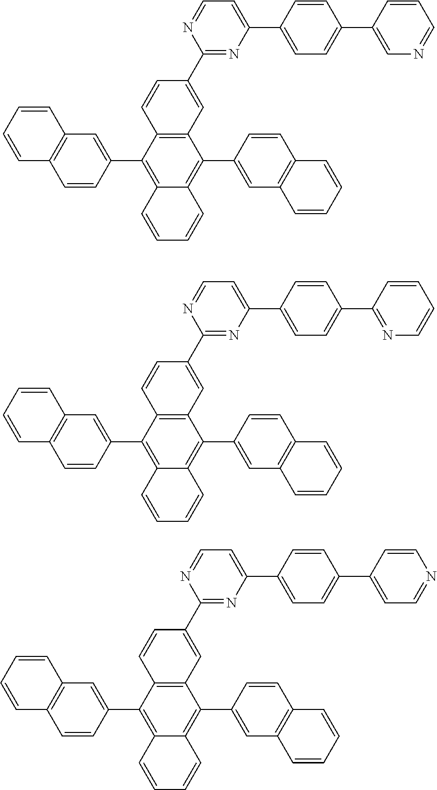

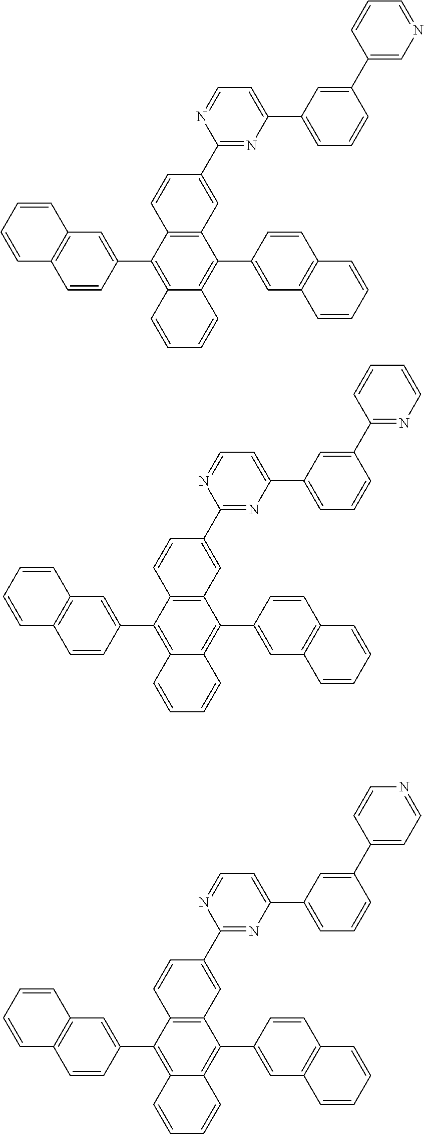 [SMILES: c1cncc(-c2cccc(-c3ccnc(-c4ccc5c(-c6ccc7ccccc7c6)c6ccccc6c(-c6ccc7ccccc7c6)c5c4)n3)c2)c1 ; c1ccc(-c2cccc(-c3ccnc(-c4ccc5c(-c6ccc7ccccc7c6)c6ccccc6c(-c6ccc7ccccc7c6)c5c4)n3)c2)nc1 ; c1cc(-c2ccncc2)cc(-c2ccnc(-c3ccc4c(-c5ccc6ccccc6c5)c5ccccc5c(-c5ccc6ccccc6c5)c4c3)n2)c1]

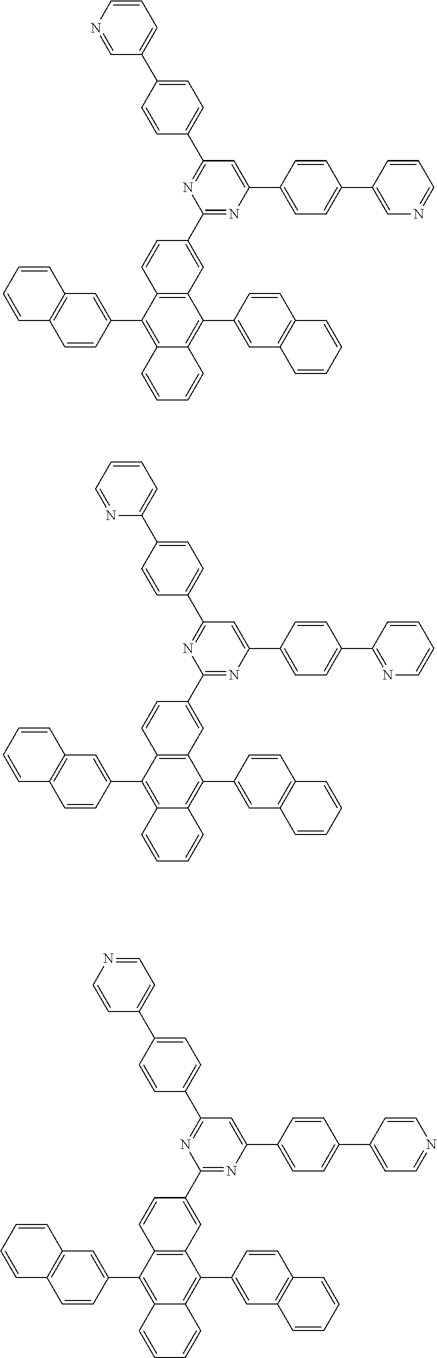 [SMILES: c1cncc(-c2ccc(-c3cc(-c4ccc(-c5cccnc5)cc4)nc(-c4ccc5c(-c6ccc7ccccc7c6)c6ccccc6c(-c6ccc7ccccc7c6)c5c4)n3)cc2)c1 ; c1ccc(-c2ccc(-c3cc(-c4ccc(-c5ccccn5)cc4)nc(-c4ccc5c(-c6ccc7ccccc7c6)c6ccccc6c(-c6ccc7ccccc7c6)c5c4)n3)cc2)nc1 ; c1ccc2cc(-c3c4ccccc4c(-c4ccc5ccccc5c4)c4cc(-c5nc(-c6ccc(-c7ccncc7)cc6)cc(-c6ccc(-c7ccncc7)cc6)n5)ccc34)ccc2c1]

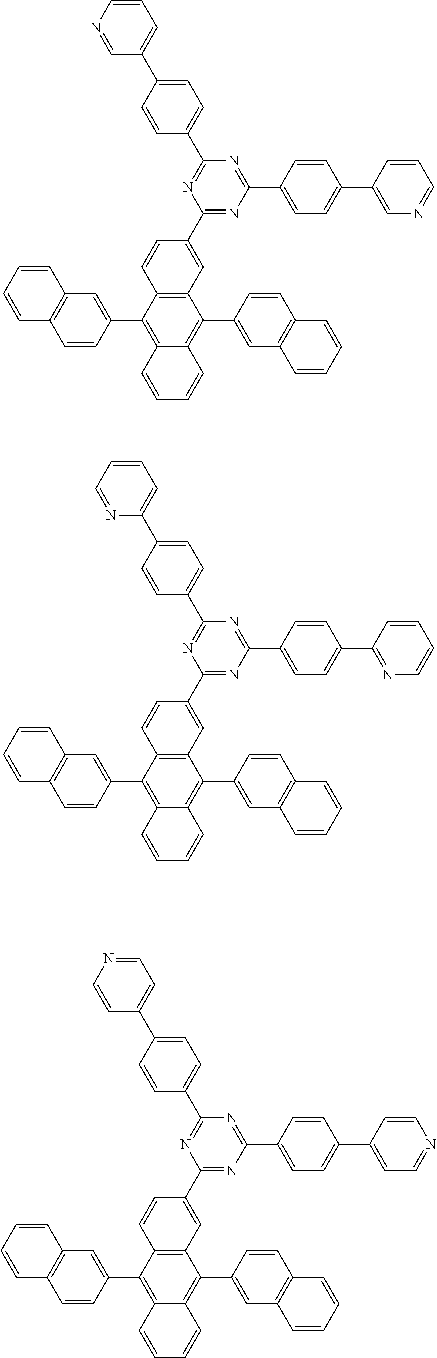 [SMILES: c1cncc(-c2ccc(-c3nc(-c4ccc(-c5cccnc5)cc4)nc(-c4ccc5c(-c6ccc7ccccc7c6)c6ccccc6c(-c6ccc7ccccc7c6)c5c4)n3)cc2)c1 ; c1ccc(-c2ccc(-c3nc(-c4ccc(-c5ccccn5)cc4)nc(-c4ccc5c(-c6ccc7ccccc7c6)c6ccccc6c(-c6ccc7ccccc7c6)c5c4)n3)cc2)nc1 ; c1ccc2cc(-c3c4ccccc4c(-c4ccc5ccccc5c4)c4cc(-c5nc(-c6ccc(-c7ccncc7)cc6)nc(-c6ccc(-c7ccncc7)cc6)n5)ccc34)ccc2c1]

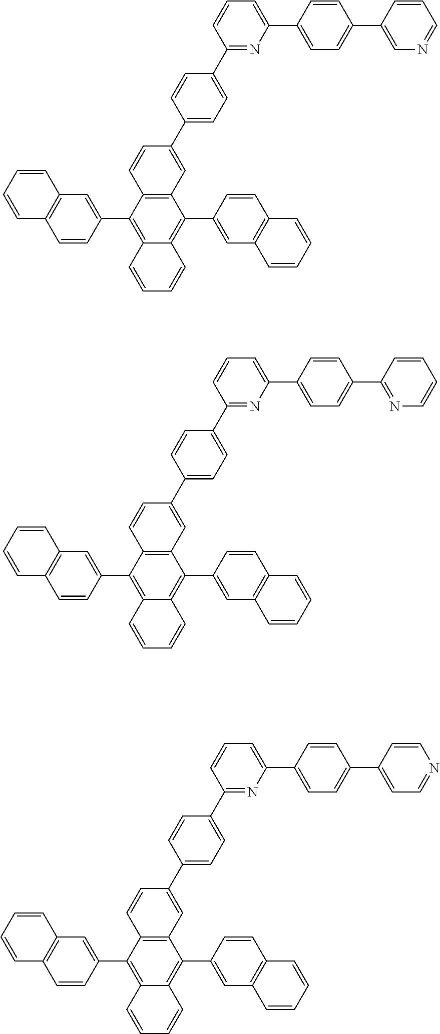 [SMILES: c1cncc(-c2ccc(-c3cccc(-c4ccc(-c5ccc6c(-c7ccc8ccccc8c7)c7ccccc7c(-c7ccc8ccccc8c7)c6c5)cc4)n3)cc2)c1 ; c1ccc(-c2ccc(-c3cccc(-c4ccc(-c5ccc6c(-c7ccc8ccccc8c7)c7ccccc7c(-c7ccc8ccccc8c7)c6c5)cc4)n3)cc2)nc1 ; c1cc(-c2ccc(-c3ccncc3)cc2)nc(-c2ccc(-c3ccc4c(-c5ccc6ccccc6c5)c5ccccc5c(-c5ccc6ccccc6c5)c4c3)cc2)c1]

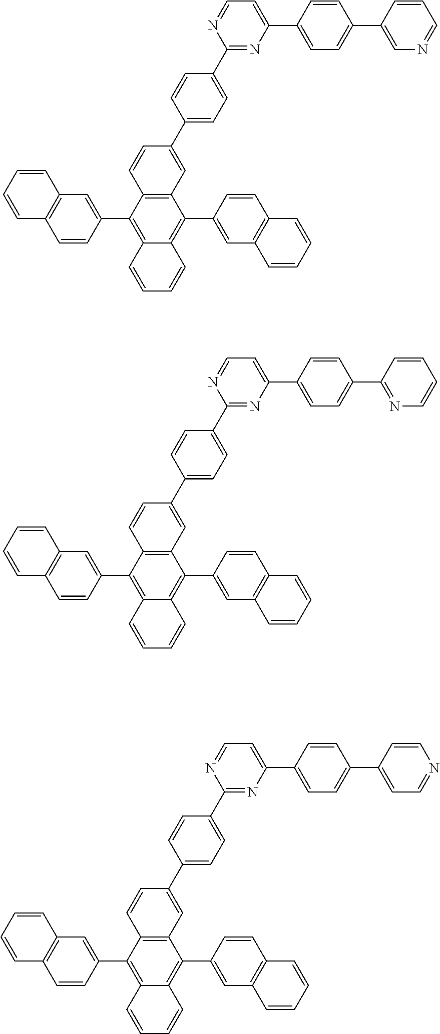 [SMILES: c1cncc(-c2ccc(-c3ccnc(-c4ccc(-c5ccc6c(-c7ccc8ccccc8c7)c7ccccc7c(-c7ccc8ccccc8c7)c6c5)cc4)n3)cc2)c1 ; c1ccc(-c2ccc(-c3ccnc(-c4ccc(-c5ccc6c(-c7ccc8ccccc8c7)c7ccccc7c(-c7ccc8ccccc8c7)c6c5)cc4)n3)cc2)nc1 ; c1ccc2cc(-c3c4ccccc4c(-c4ccc5ccccc5c4)c4cc(-c5ccc(-c6nccc(-c7ccc(-c8ccncc8)cc7)n6)cc5)ccc34)ccc2c1]

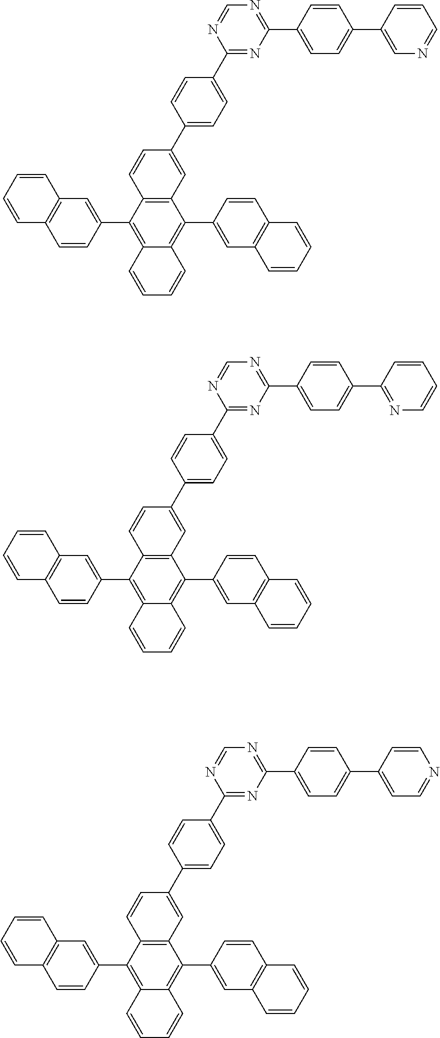 [SMILES: c1cncc(-c2ccc(-c3ncnc(-c4ccc(-c5ccc6c(-c7ccc8ccccc8c7)c7ccccc7c(-c7ccc8ccccc8c7)c6c5)cc4)n3)cc2)c1 ; c1ccc(-c2ccc(-c3ncnc(-c4ccc(-c5ccc6c(-c7ccc8ccccc8c7)c7ccccc7c(-c7ccc8ccccc8c7)c6c5)cc4)n3)cc2)nc1 ; c1ccc2cc(-c3c4ccccc4c(-c4ccc5ccccc5c4)c4cc(-c5ccc(-c6ncnc(-c7ccc(-c8ccncc8)cc7)n6)cc5)ccc34)ccc2c1]

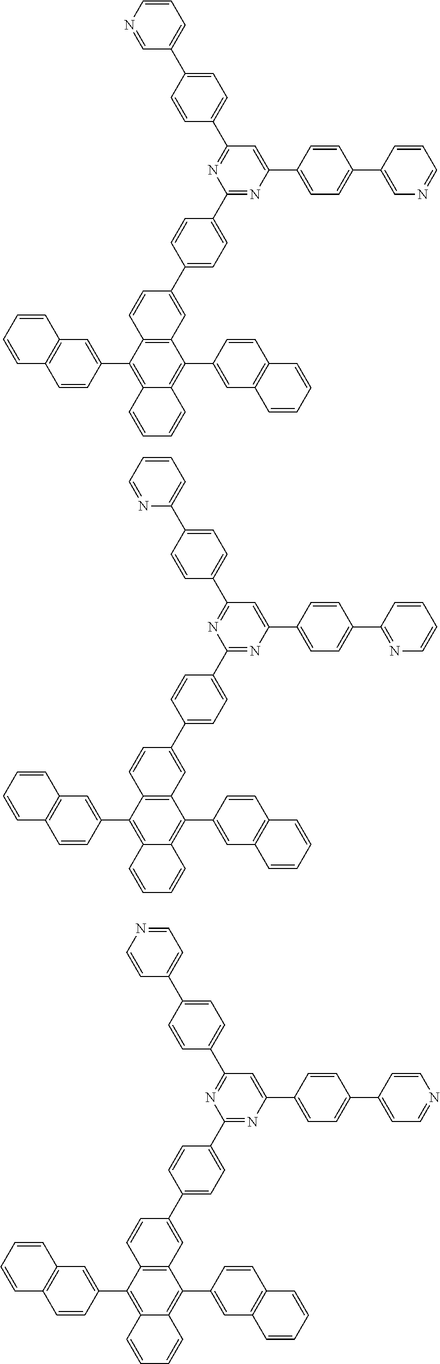

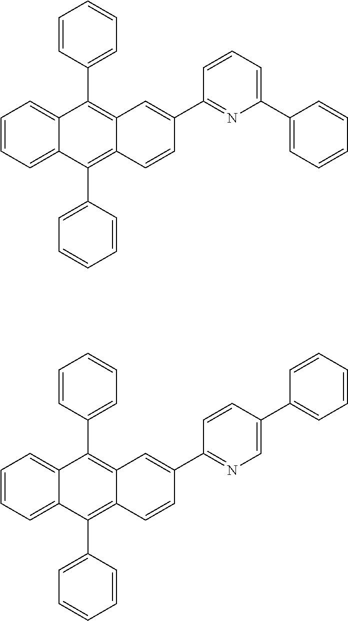 [SMILES: c1ccc(-c2cccc(-c3ccc4c(-c5ccccc5)c5ccccc5c(-c5ccccc5)c4c3)n2)cc1 ; c1ccc(-c2ccc(-c3ccc4c(-c5ccccc5)c5ccccc5c(-c5ccccc5)c4c3)nc2)cc1]

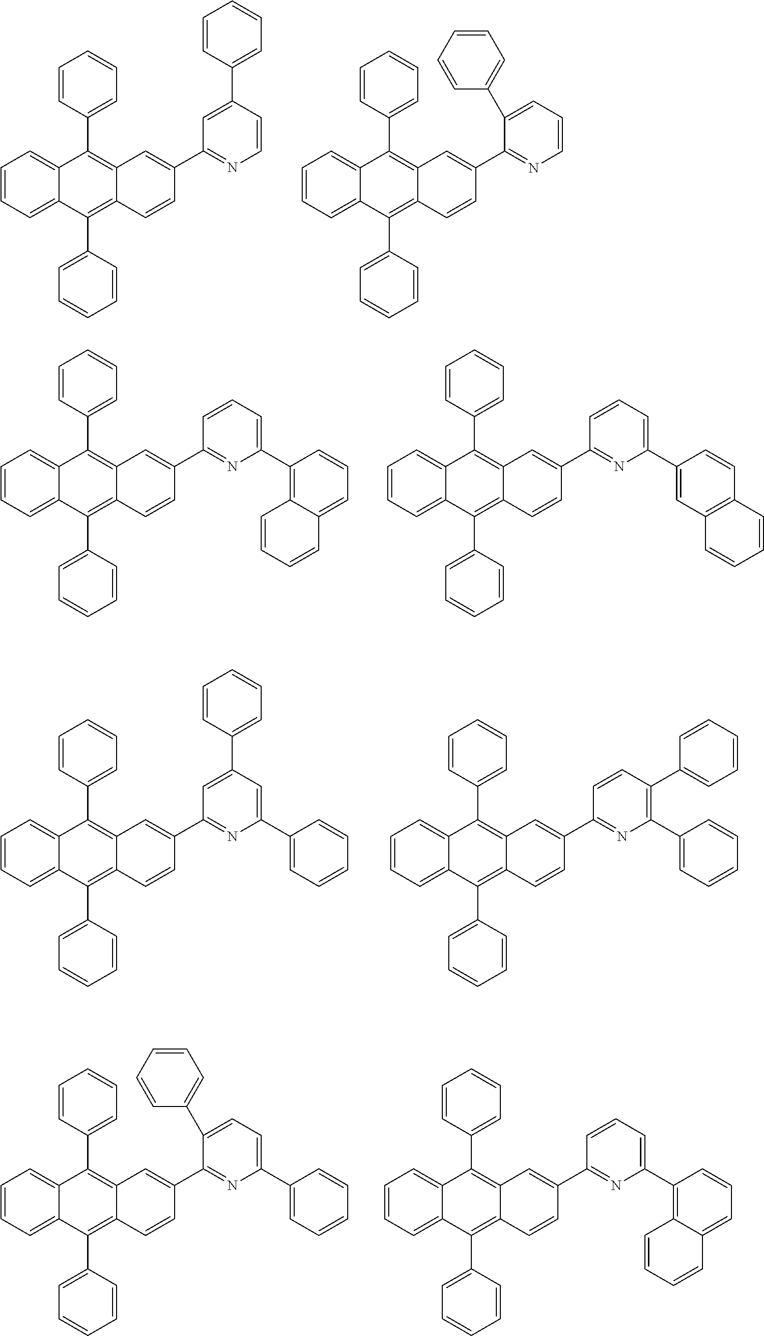

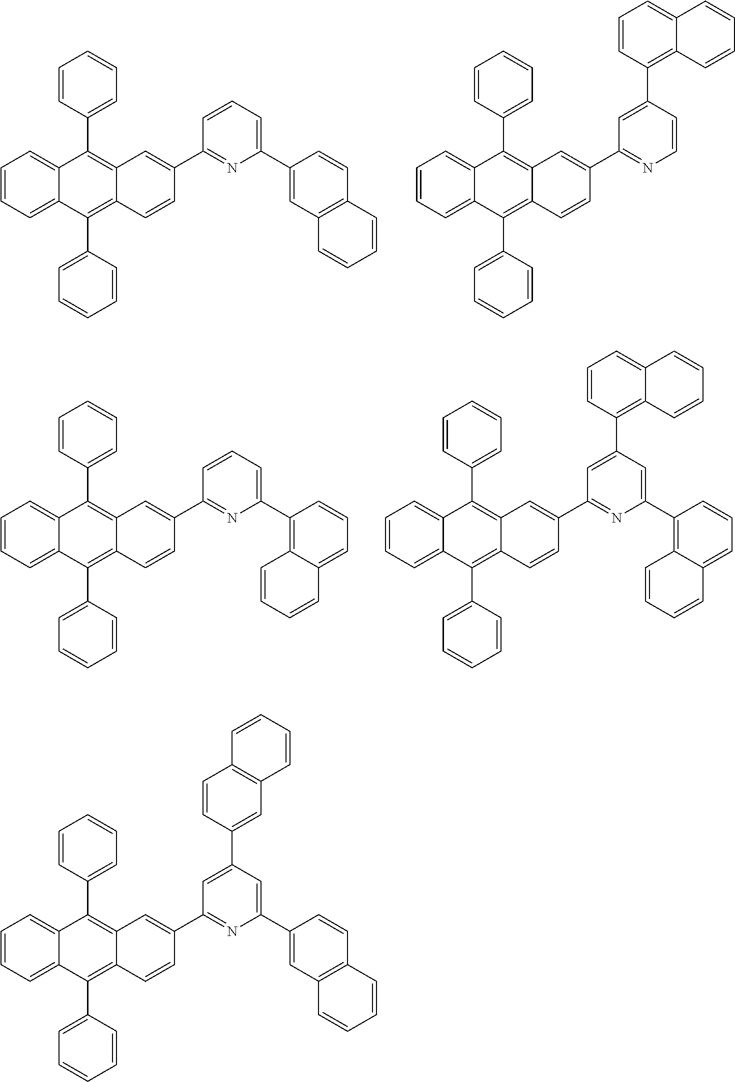

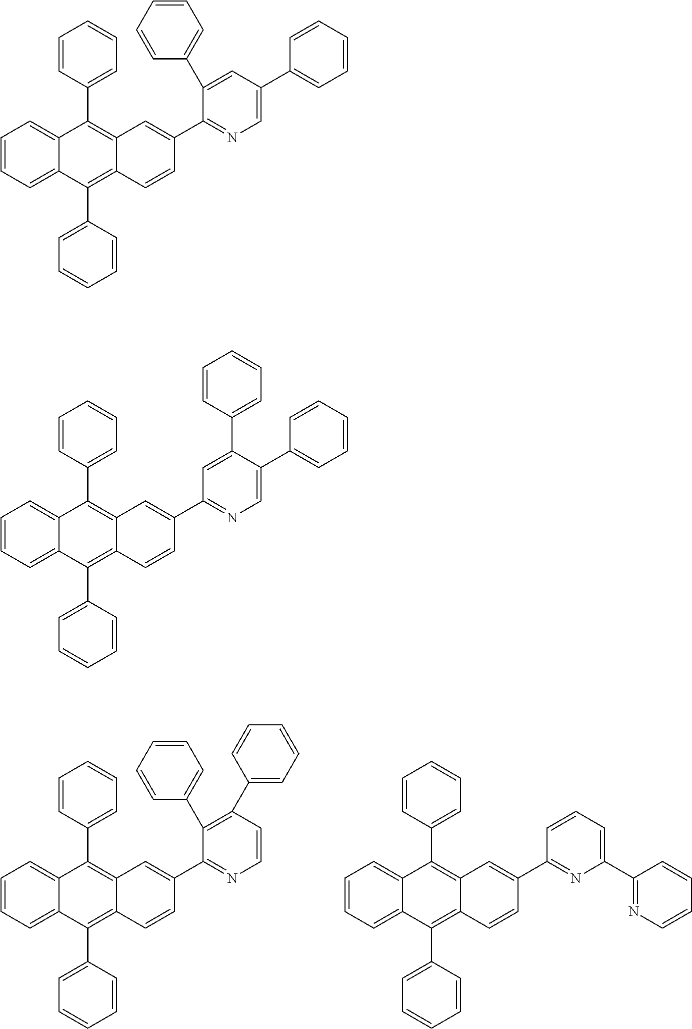 [SMILES: c1ccc(-c2cnc(-c3ccc4c(-c5ccccc5)c5ccccc5c(-c5ccccc5)c4c3)c(-c3ccccc3)c2)cc1 ; c1ccc(-c2cnc(-c3ccc4c(-c5ccccc5)c5ccccc5c(-c5ccccc5)c4c3)cc2-c2ccccc2)cc1 ; c1ccc(-c2ccnc(-c3ccc4c(-c5ccccc5)c5ccccc5c(-c5ccccc5)c4c3)c2-c2ccccc2)cc1 ; c1ccc(-c2c3ccccc3c(-c3ccccc3)c3cc(-c4cccc(-c5ccccn5)n4)ccc23)cc1]

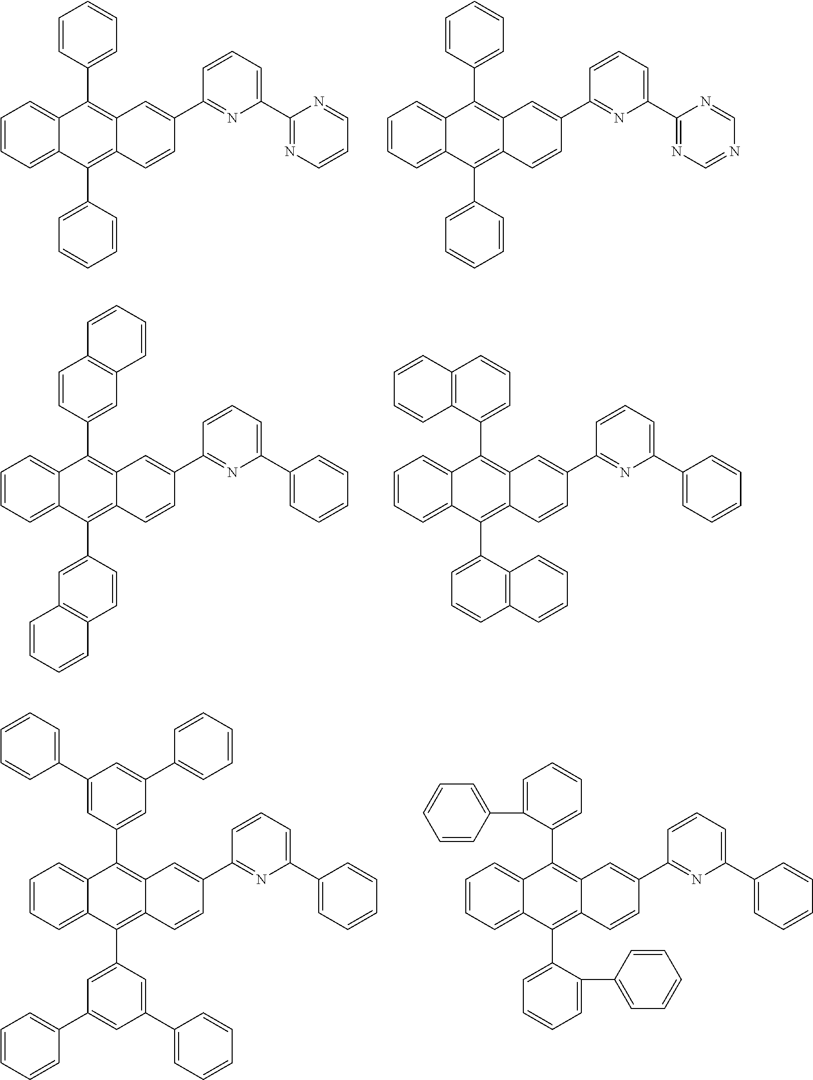

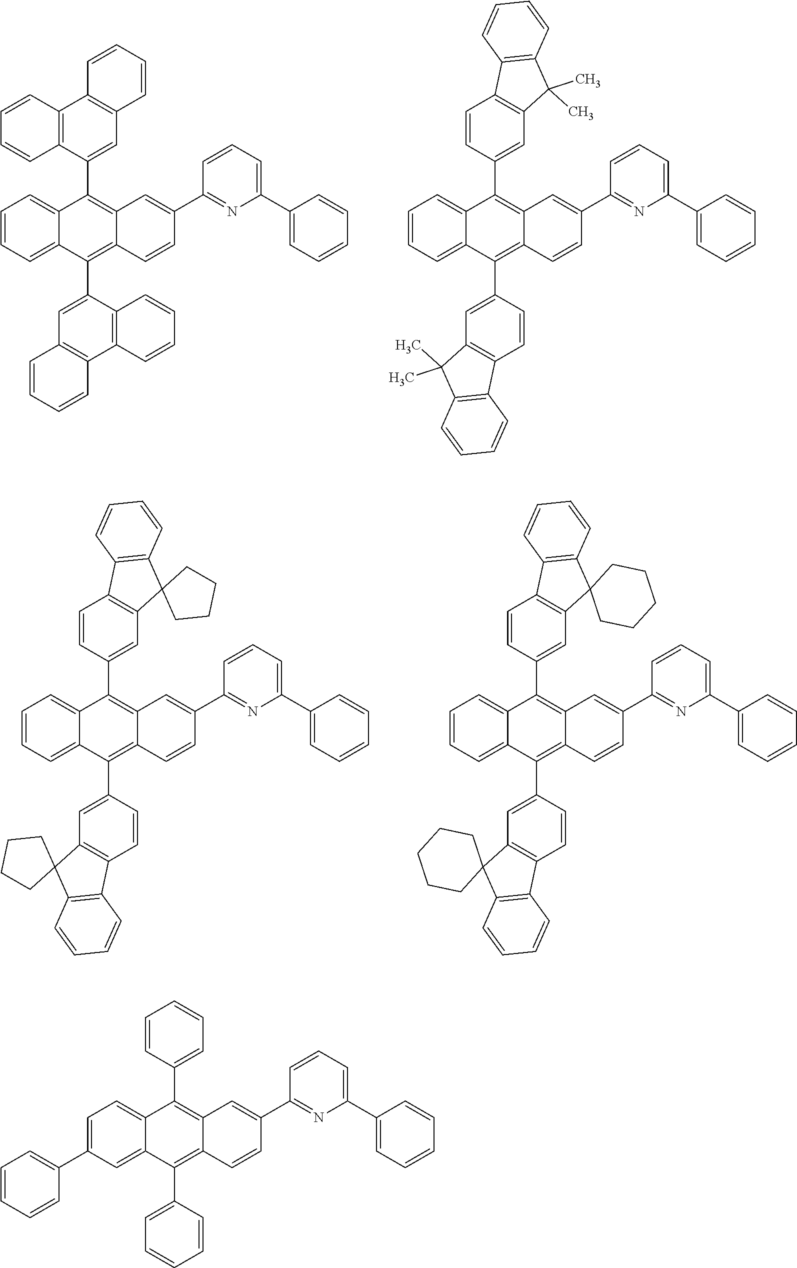 [SMILES: c1ccc(-c2cccc(-c3ccc4c(-c5cc6ccccc6c6ccccc56)c5ccccc5c(-c5cc6ccccc6c6ccccc56)c4c3)n2)cc1 ; CC1(C)c2ccccc2-c2ccc(-c3c4ccccc4c(-c4ccc5c(c4)C(C)(C)c4ccccc4-5)c4cc(-c5cccc(-c6ccccc6)n5)ccc34)cc21 ; c1ccc(-c2cccc(-c3ccc4c(-c5ccc6c(c5)C5(CCCC5)c5ccccc5-6)c5ccccc5c(-c5ccc6c(c5)C5(CCCC5)c5ccccc5-6)c4c3)n2)cc1 ; c1ccc(-c2cccc(-c3ccc4c(-c5ccc6c(c5)C5(CCCCC5)c5ccccc5-6)c5ccccc5c(-c5ccc6c(c5)C5(CCCCC5)c5ccccc5-6)c4c3)n2)cc1 ; c1ccc(-c2ccc3c(-c4ccccc4)c4cc(-c5cccc(-c6ccccc6)n5)ccc4c(-c4ccccc4)c3c2)cc1]

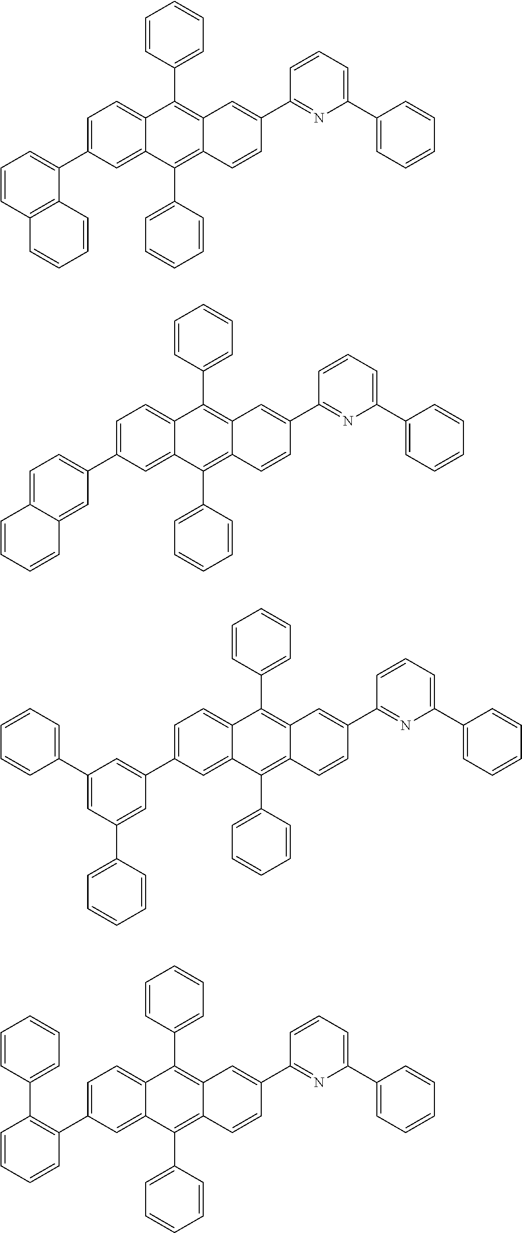

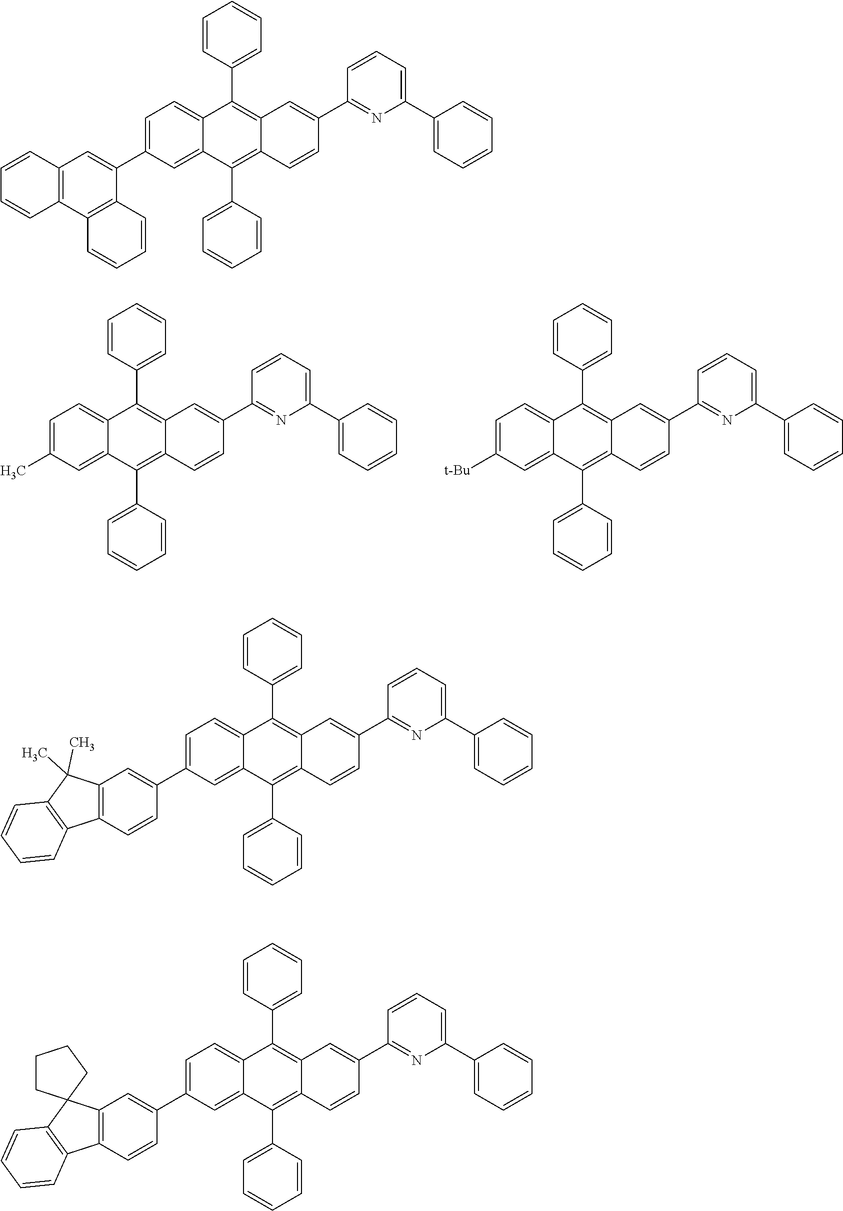

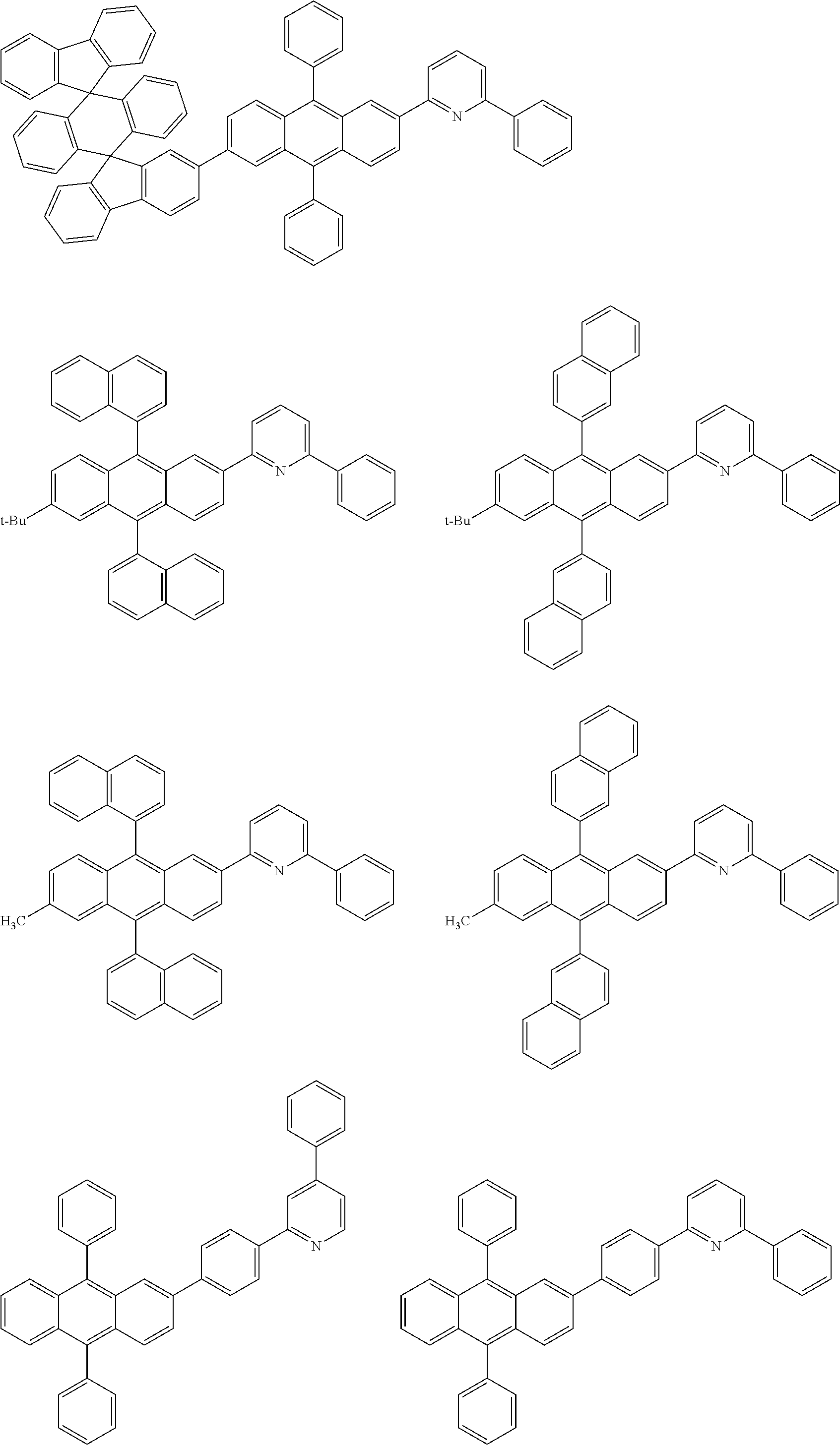

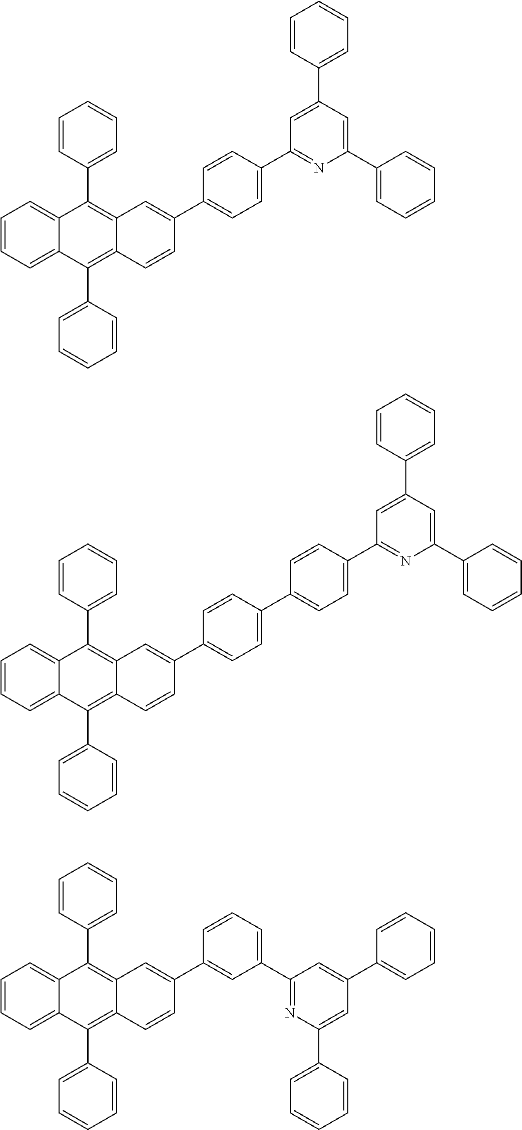 [SMILES: c1ccc(-c2cc(-c3ccccc3)nc(-c3ccc(-c4ccc5c(-c6ccccc6)c6ccccc6c(-c6ccccc6)c5c4)cc3)c2)cc1 ; c1ccc(-c2cc(-c3ccccc3)nc(-c3ccc(-c4ccc(-c5ccc6c(-c7ccccc7)c7ccccc7c(-c7ccccc7)c6c5)cc4)cc3)c2)cc1 ; c1ccc(-c2cc(-c3ccccc3)nc(-c3cccc(-c4ccc5c(-c6ccccc6)c6ccccc6c(-c6ccccc6)c5c4)c3)c2)cc1]

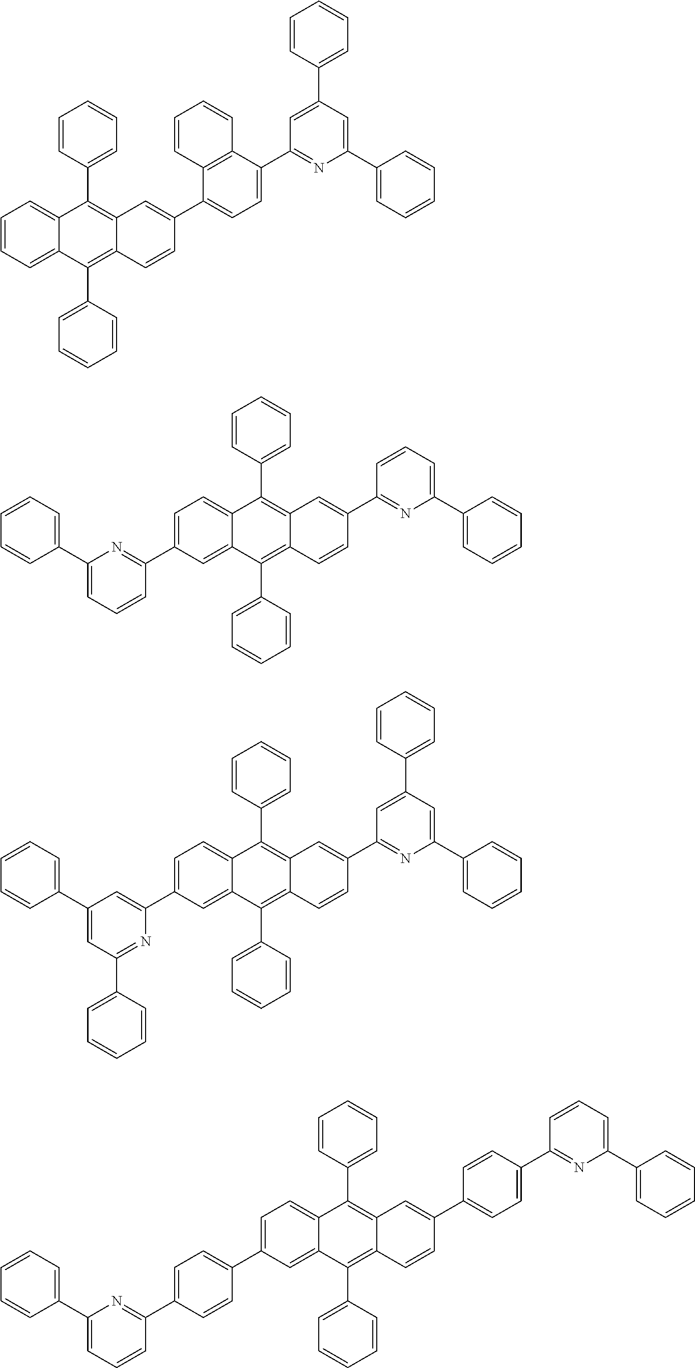 [SMILES: c1ccc(-c2cc(-c3ccccc3)nc(-c3ccc(-c4ccc5c(-c6ccccc6)c6ccccc6c(-c6ccccc6)c5c4)c4ccccc34)c2)cc1 ; c1ccc(-c2cccc(-c3ccc4c(-c5ccccc5)c5cc(-c6cccc(-c7ccccc7)n6)ccc5c(-c5ccccc5)c4c3)n2)cc1 ; c1ccc(-c2cc(-c3ccccc3)nc(-c3ccc4c(-c5ccccc5)c5cc(-c6cc(-c7ccccc7)cc(-c7ccccc7)n6)ccc5c(-c5ccccc5)c4c3)c2)cc1 ; c1ccc(-c2cccc(-c3ccc(-c4ccc5c(-c6ccccc6)c6cc(-c7ccc(-c8cccc(-c9ccccc9)n8)cc7)ccc6c(-c6ccccc6)c5c4)cc3)n2)cc1]

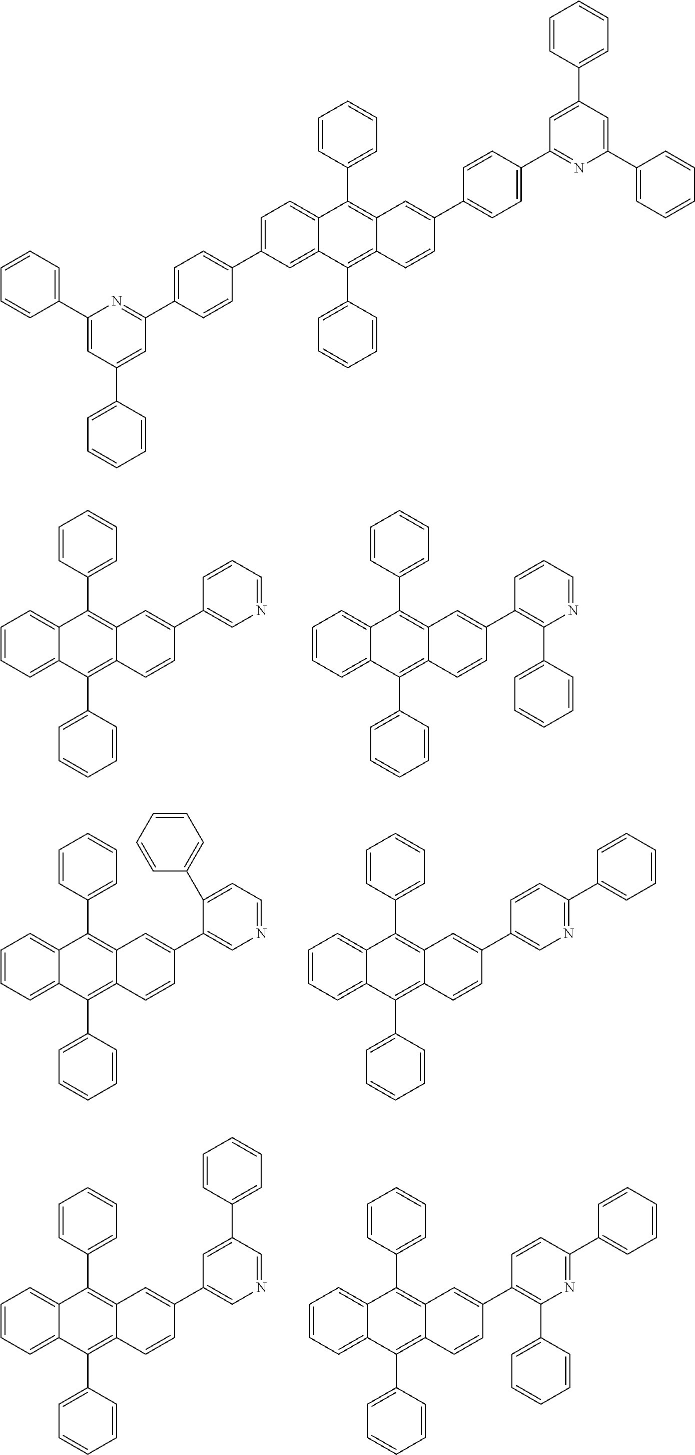

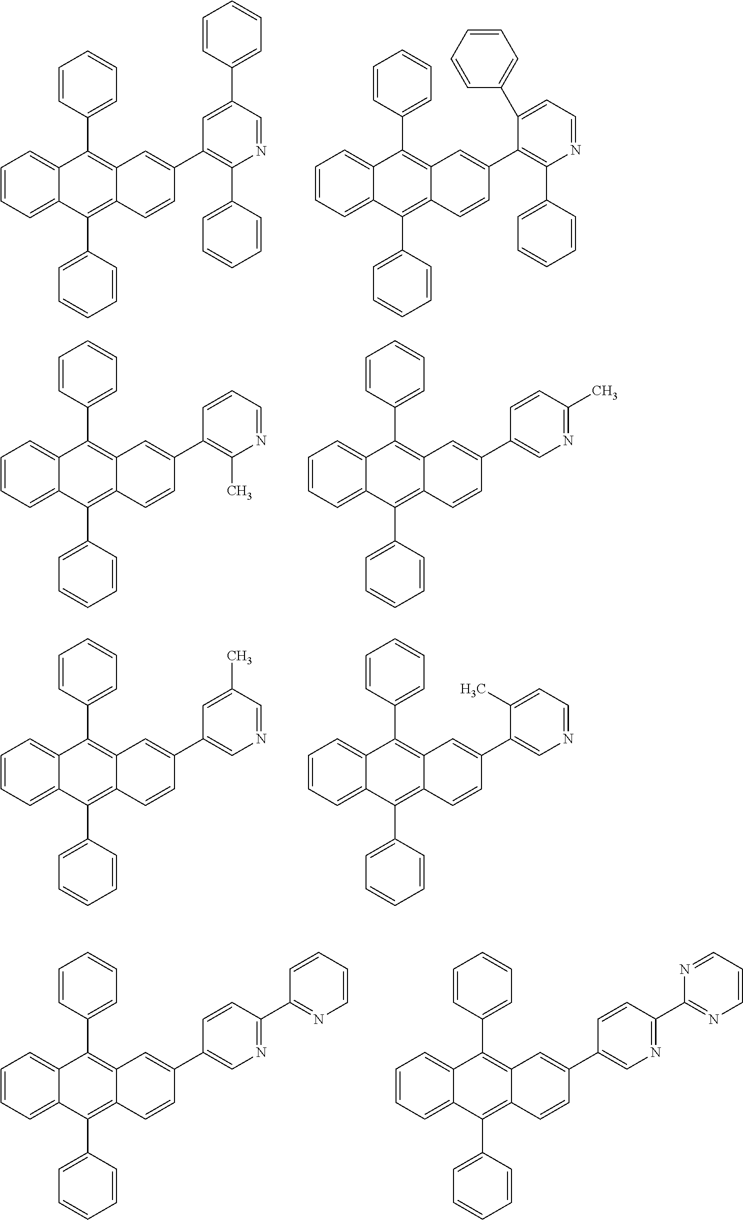

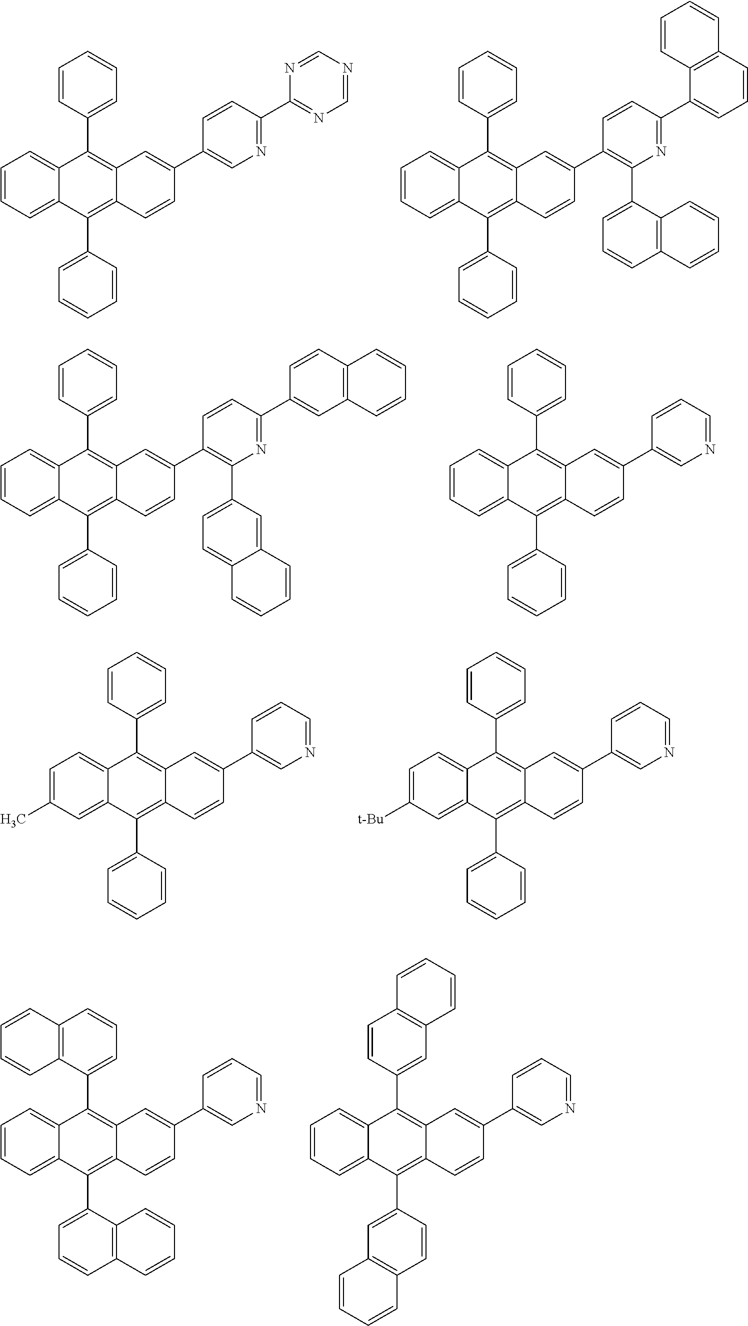

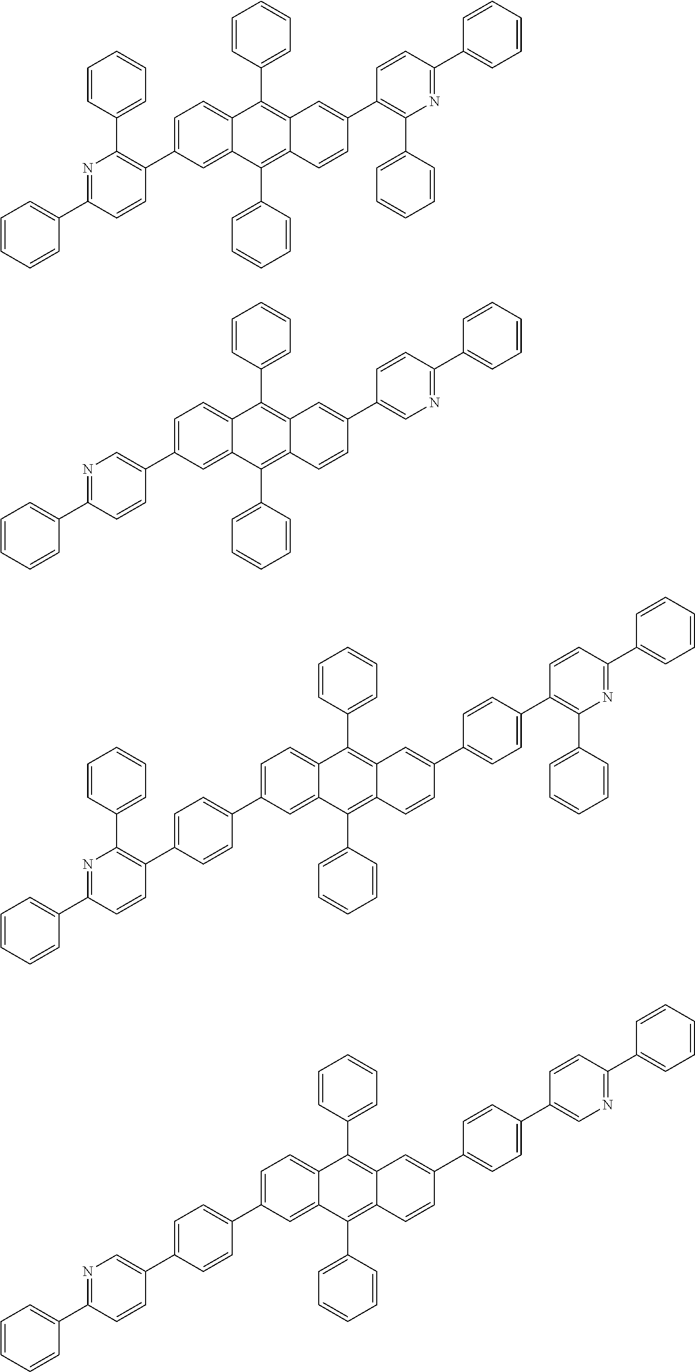

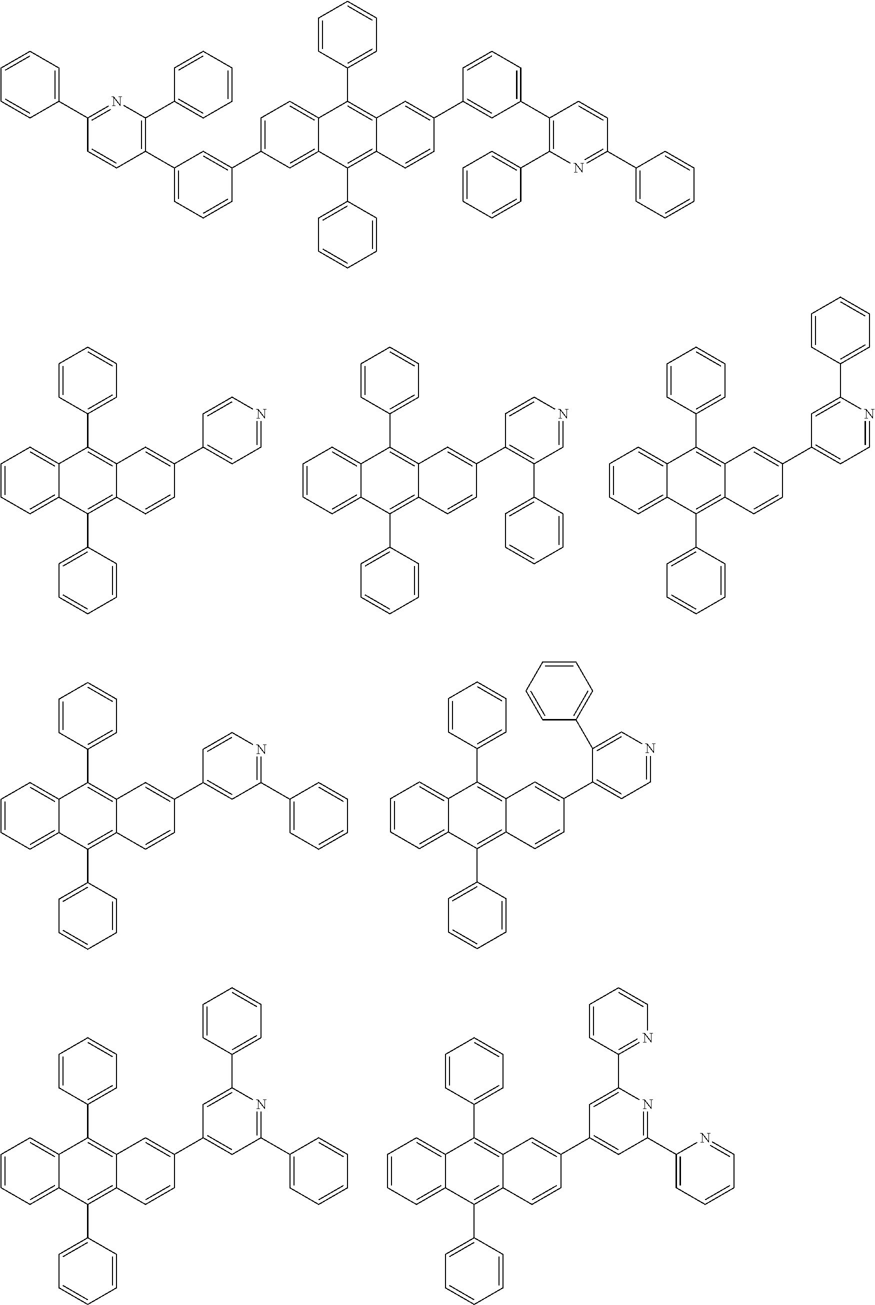

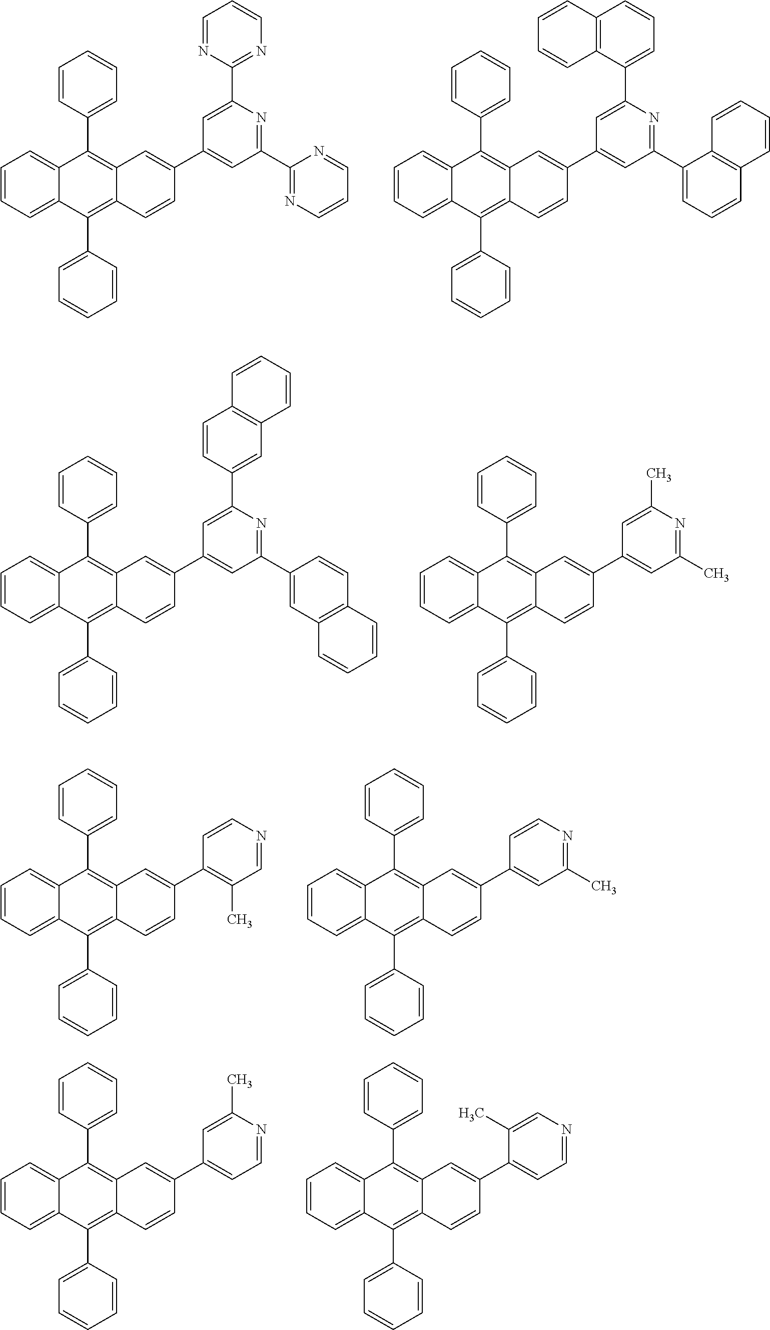

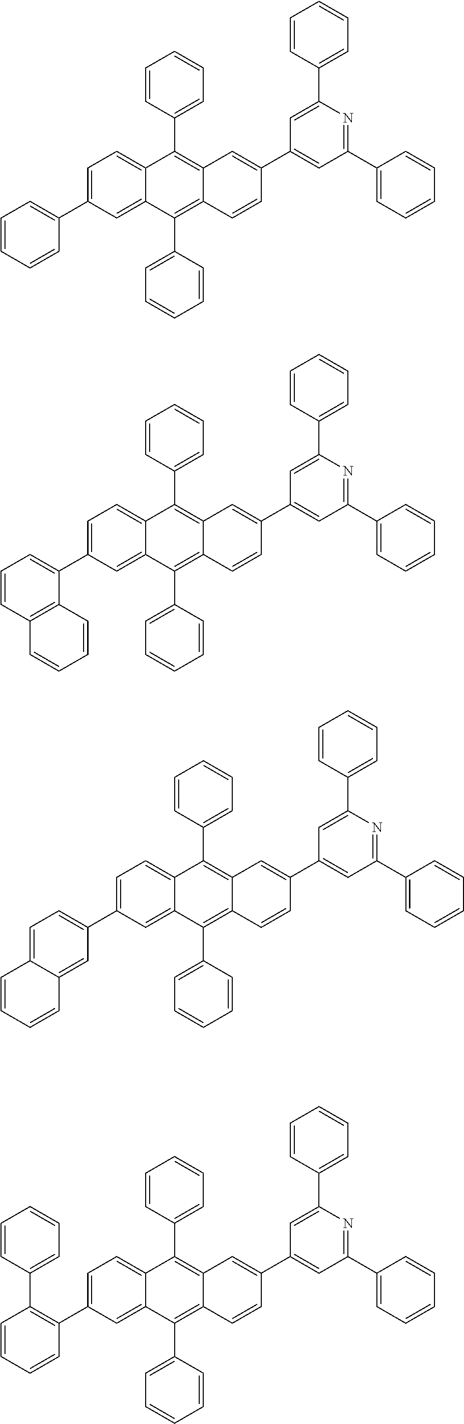 [SMILES: c1ccc(-c2ccc3c(-c4ccccc4)c4cc(-c5cc(-c6ccccc6)nc(-c6ccccc6)c5)ccc4c(-c4ccccc4)c3c2)cc1 ; c1ccc(-c2cc(-c3ccc4c(-c5ccccc5)c5cc(-c6cccc7ccccc67)ccc5c(-c5ccccc5)c4c3)cc(-c3ccccc3)n2)cc1 ; c1ccc(-c2cc(-c3ccc4c(-c5ccccc5)c5cc(-c6ccc7ccccc7c6)ccc5c(-c5ccccc5)c4c3)cc(-c3ccccc3)n2)cc1 ; c1ccc(-c2cc(-c3ccc4c(-c5ccccc5)c5cc(-c6ccccc6-c6ccccc6)ccc5c(-c5ccccc5)c4c3)cc(-c3ccccc3)n2)cc1]

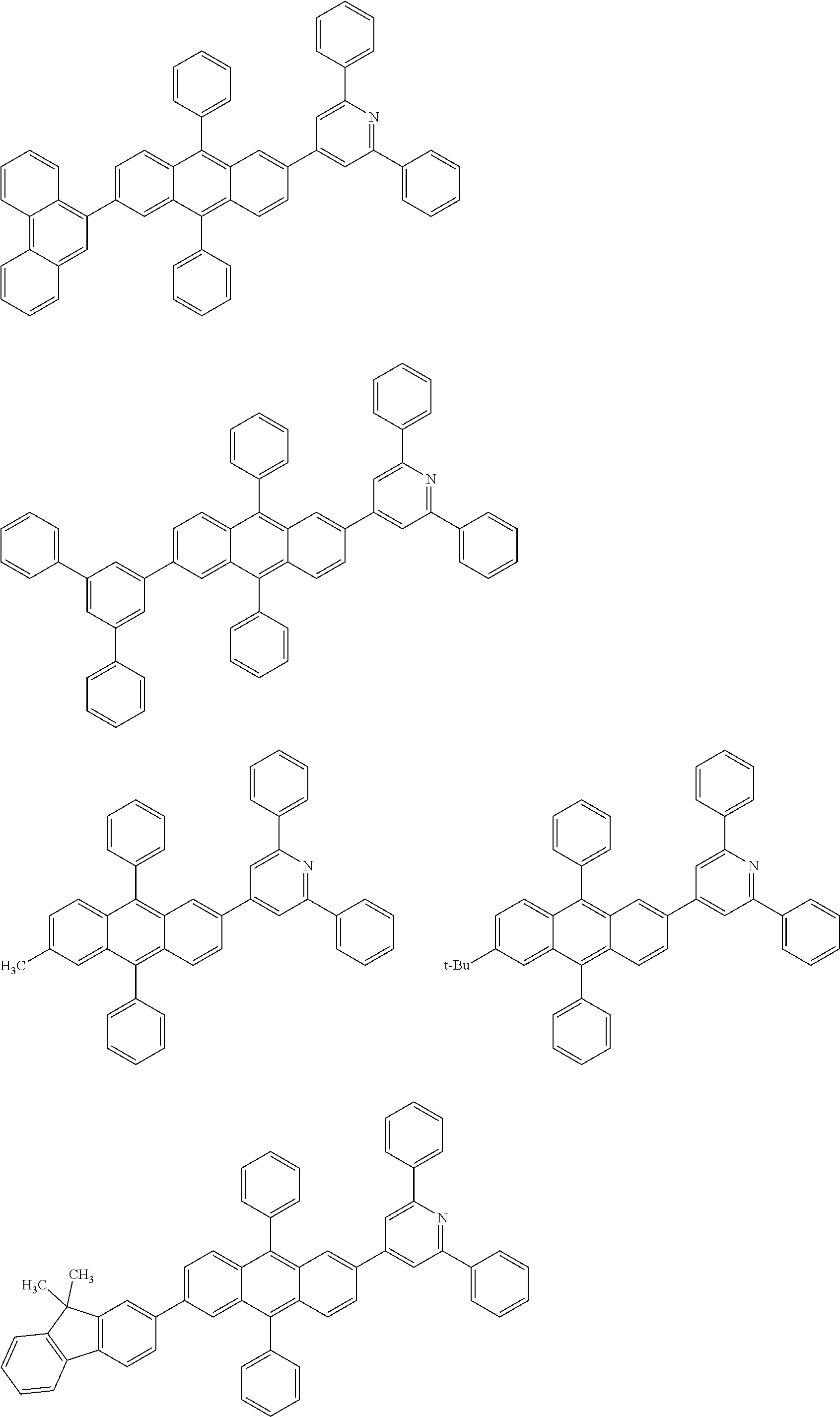 [SMILES: c1ccc(-c2cc(-c3ccc4c(-c5ccccc5)c5cc(-c6cc7ccccc7c7ccccc67)ccc5c(-c5ccccc5)c4c3)cc(-c3ccccc3)n2)cc1 ; c1ccc(-c2cc(-c3ccccc3)cc(-c3ccc4c(-c5ccccc5)c5cc(-c6cc(-c7ccccc7)nc(-c7ccccc7)c6)ccc5c(-c5ccccc5)c4c3)c2)cc1 ; Cc1ccc2c(-c3ccccc3)c3cc(-c4cc(-c5ccccc5)nc(-c5ccccc5)c4)ccc3c(-c3ccccc3)c2c1 ; CC(C)(C)c1ccc2c(-c3ccccc3)c3cc(-c4cc(-c5ccccc5)nc(-c5ccccc5)c4)ccc3c(-c3ccccc3)c2c1 ; CC1(C)c2ccccc2-c2ccc(-c3ccc4c(-c5ccccc5)c5cc(-c6cc(-c7ccccc7)nc(-c7ccccc7)c6)ccc5c(-c5ccccc5)c4c3)cc21]

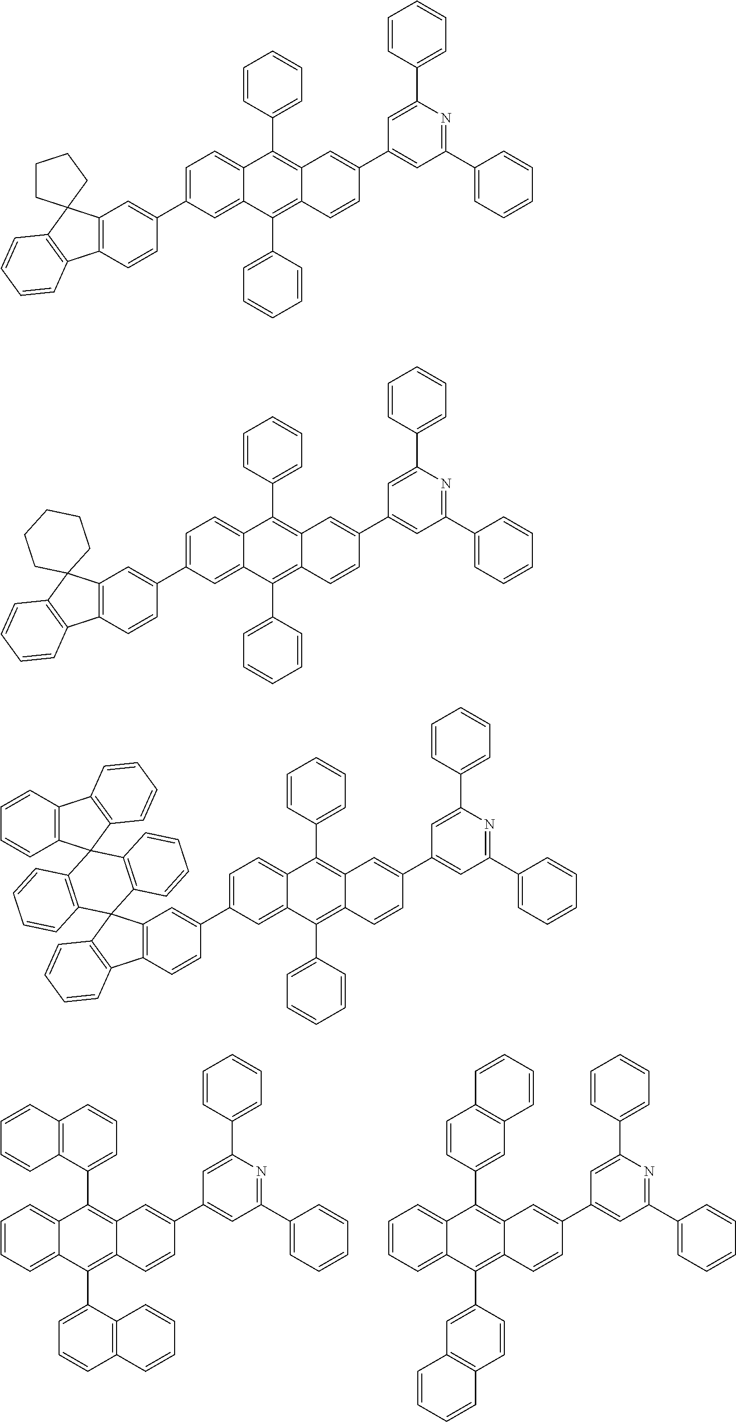

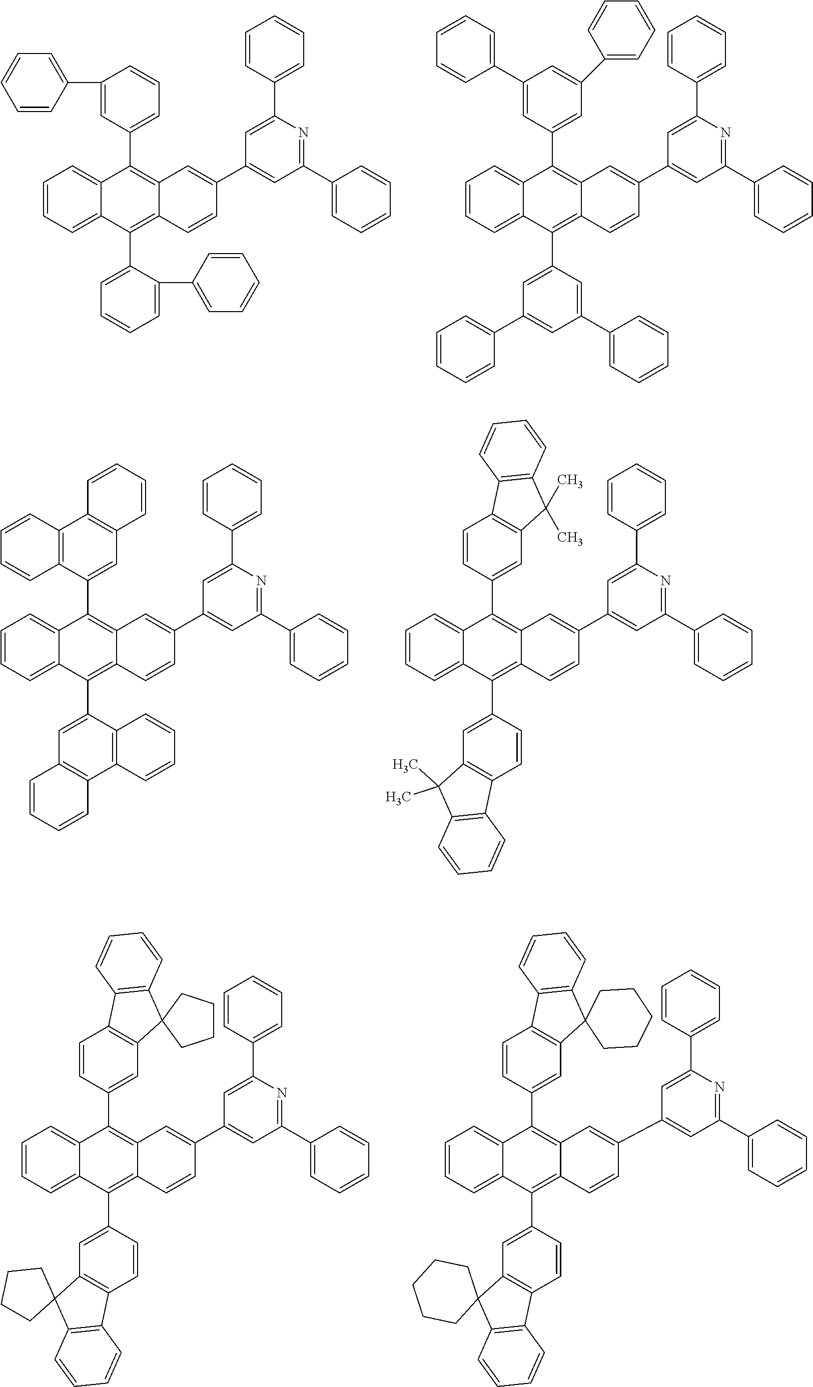

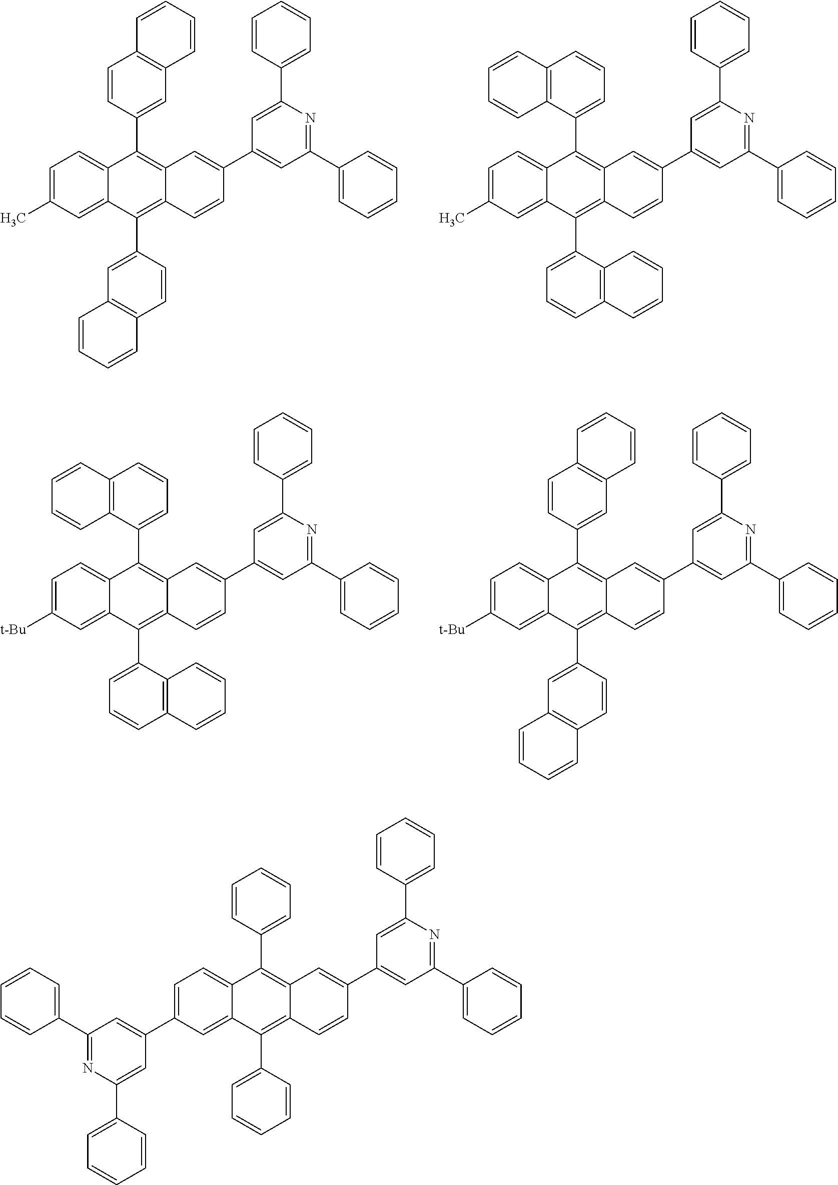 [SMILES: Cc1ccc2c(-c3ccc4ccccc4c3)c3cc(-c4cc(-c5ccccc5)nc(-c5ccccc5)c4)ccc3c(-c3ccc4ccccc4c3)c2c1 ; Cc1ccc2c(-c3cccc4ccccc34)c3cc(-c4cc(-c5ccccc5)nc(-c5ccccc5)c4)ccc3c(-c3cccc4ccccc34)c2c1 ; CC(C)(C)c1ccc2c(-c3cccc4ccccc34)c3cc(-c4cc(-c5ccccc5)nc(-c5ccccc5)c4)ccc3c(-c3cccc4ccccc34)c2c1 ; CC(C)(C)c1ccc2c(-c3ccc4ccccc4c3)c3cc(-c4cc(-c5ccccc5)nc(-c5ccccc5)c4)ccc3c(-c3ccc4ccccc4c3)c2c1 ; c1ccc(-c2cc(-c3ccc4c(-c5ccccc5)c5cc(-c6cc(-c7ccccc7)nc(-c7ccccc7)c6)ccc5c(-c5ccccc5)c4c3)cc(-c3ccccc3)n2)cc1]

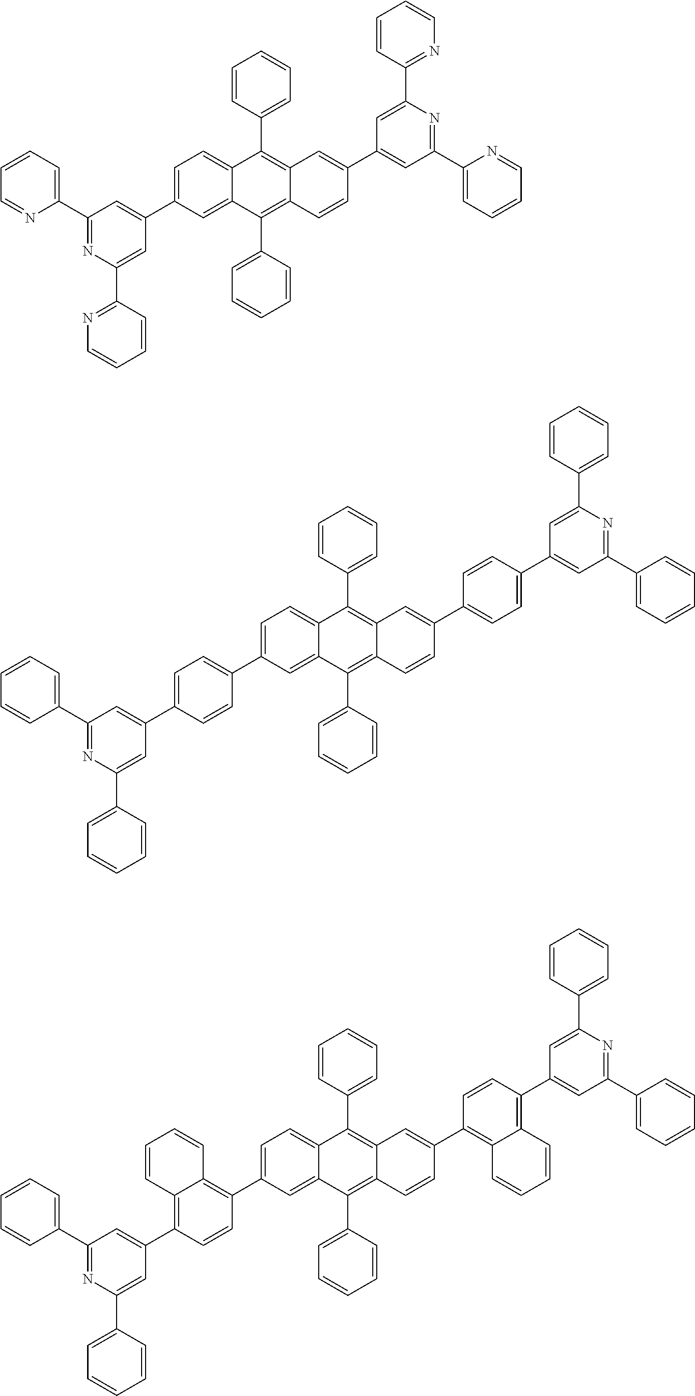 [SMILES: c1ccc(-c2c3ccc(-c4cc(-c5ccccn5)nc(-c5ccccn5)c4)cc3c(-c3ccccc3)c3ccc(-c4cc(-c5ccccn5)nc(-c5ccccn5)c4)cc23)cc1 ; c1ccc(-c2cc(-c3ccc(-c4ccc5c(-c6ccccc6)c6cc(-c7ccc(-c8cc(-c9ccccc9)nc(-c9ccccc9)c8)cc7)ccc6c(-c6ccccc6)c5c4)cc3)cc(-c3ccccc3)n2)cc1 ; c1ccc(-c2cc(-c3ccc(-c4ccc5c(-c6ccccc6)c6cc(-c7ccc(-c8cc(-c9ccccc9)nc(-c9ccccc9)c8)c8ccccc78)ccc6c(-c6ccccc6)c5c4)c4ccccc34)cc(-c3ccccc3)n2)cc1]

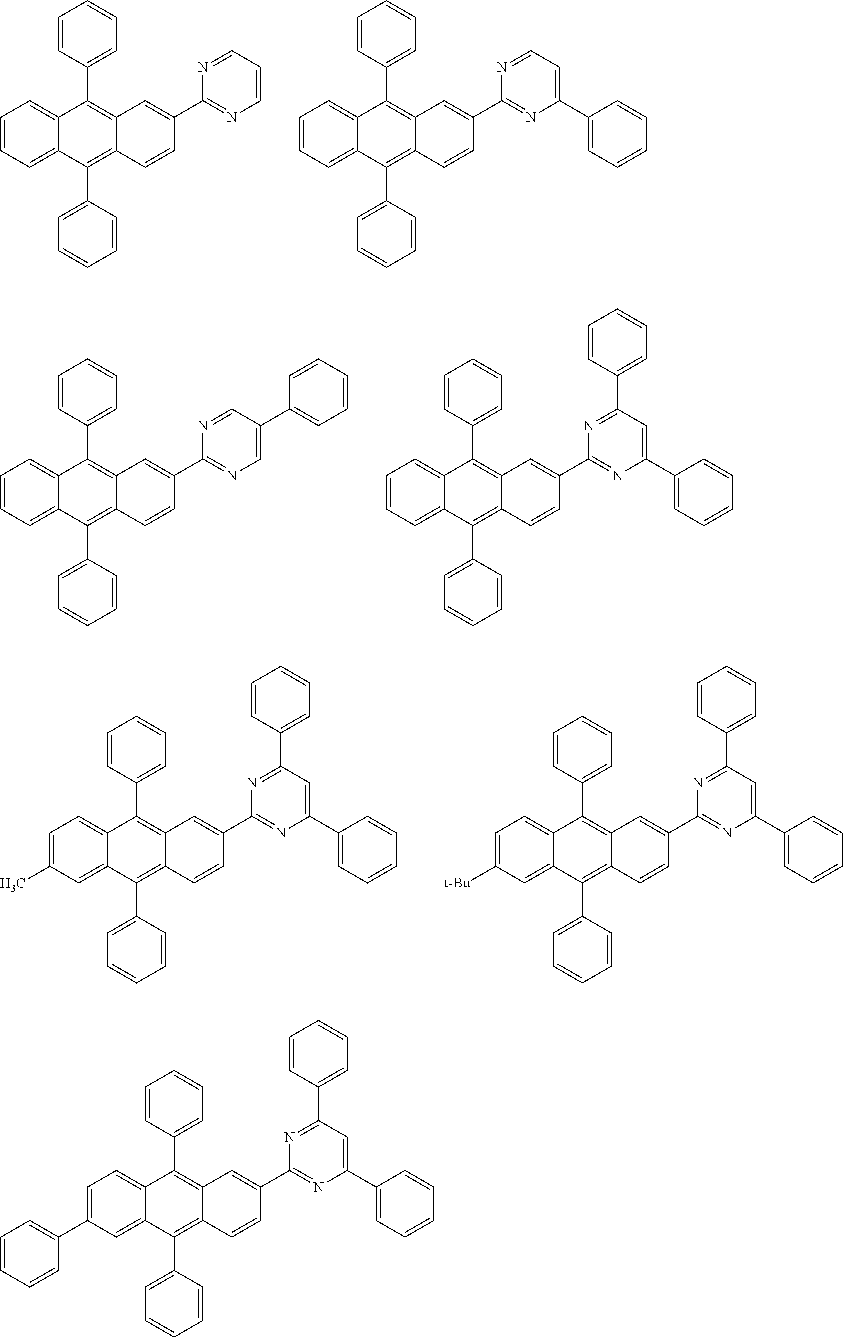

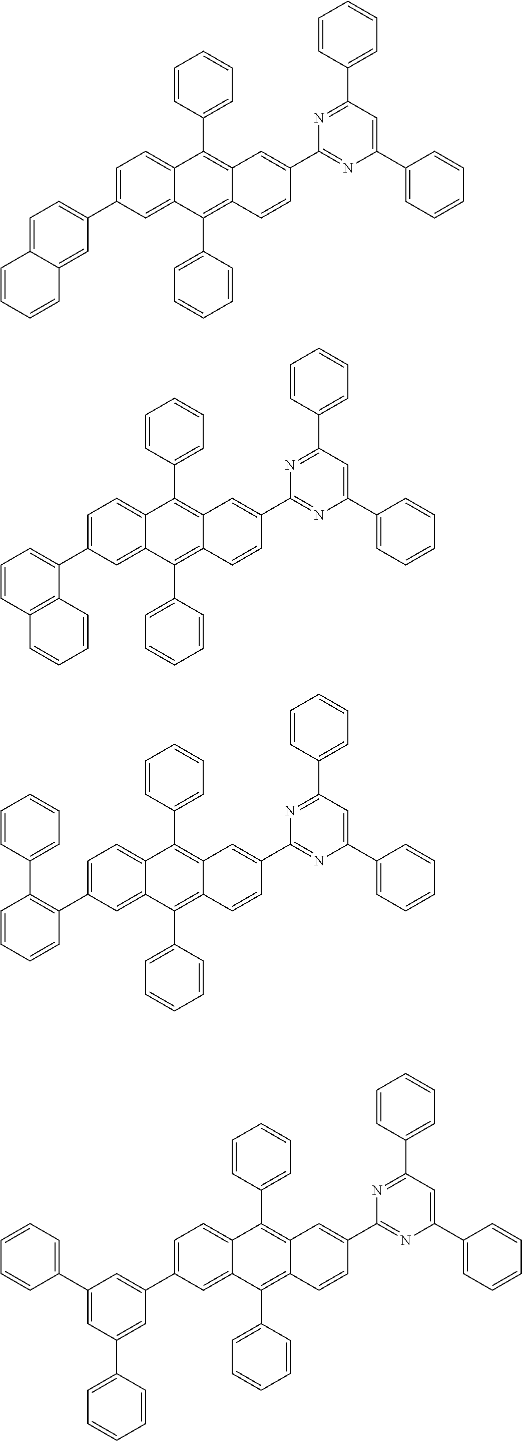 [SMILES: c1ccc(-c2cc(-c3ccccc3)nc(-c3ccc4c(-c5ccccc5)c5cc(-c6ccc7ccccc7c6)ccc5c(-c5ccccc5)c4c3)n2)cc1 ; c1ccc(-c2cc(-c3ccccc3)nc(-c3ccc4c(-c5ccccc5)c5cc(-c6cccc7ccccc67)ccc5c(-c5ccccc5)c4c3)n2)cc1 ; c1ccc(-c2cc(-c3ccccc3)nc(-c3ccc4c(-c5ccccc5)c5cc(-c6ccccc6-c6ccccc6)ccc5c(-c5ccccc5)c4c3)n2)cc1 ; c1ccc(-c2cc(-c3ccccc3)cc(-c3ccc4c(-c5ccccc5)c5cc(-c6nc(-c7ccccc7)cc(-c7ccccc7)n6)ccc5c(-c5ccccc5)c4c3)c2)cc1]

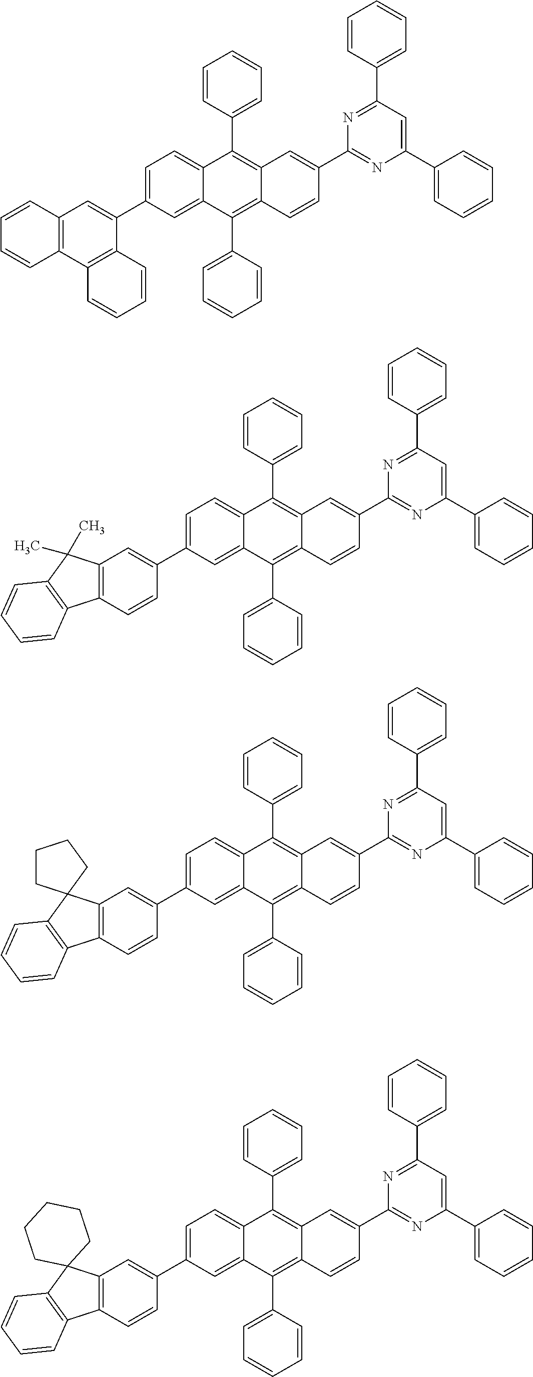 [SMILES: c1ccc(-c2cc(-c3ccccc3)nc(-c3ccc4c(-c5ccccc5)c5cc(-c6cc7ccccc7c7ccccc67)ccc5c(-c5ccccc5)c4c3)n2)cc1 ; CC1(C)c2ccccc2-c2ccc(-c3ccc4c(-c5ccccc5)c5cc(-c6nc(-c7ccccc7)cc(-c7ccccc7)n6)ccc5c(-c5ccccc5)c4c3)cc21 ; c1ccc(-c2cc(-c3ccccc3)nc(-c3ccc4c(-c5ccccc5)c5cc(-c6ccc7c(c6)C6(CCCC6)c6ccccc6-7)ccc5c(-c5ccccc5)c4c3)n2)cc1 ; c1ccc(-c2cc(-c3ccccc3)nc(-c3ccc4c(-c5ccccc5)c5cc(-c6ccc7c(c6)C6(CCCCC6)c6ccccc6-7)ccc5c(-c5ccccc5)c4c3)n2)cc1]

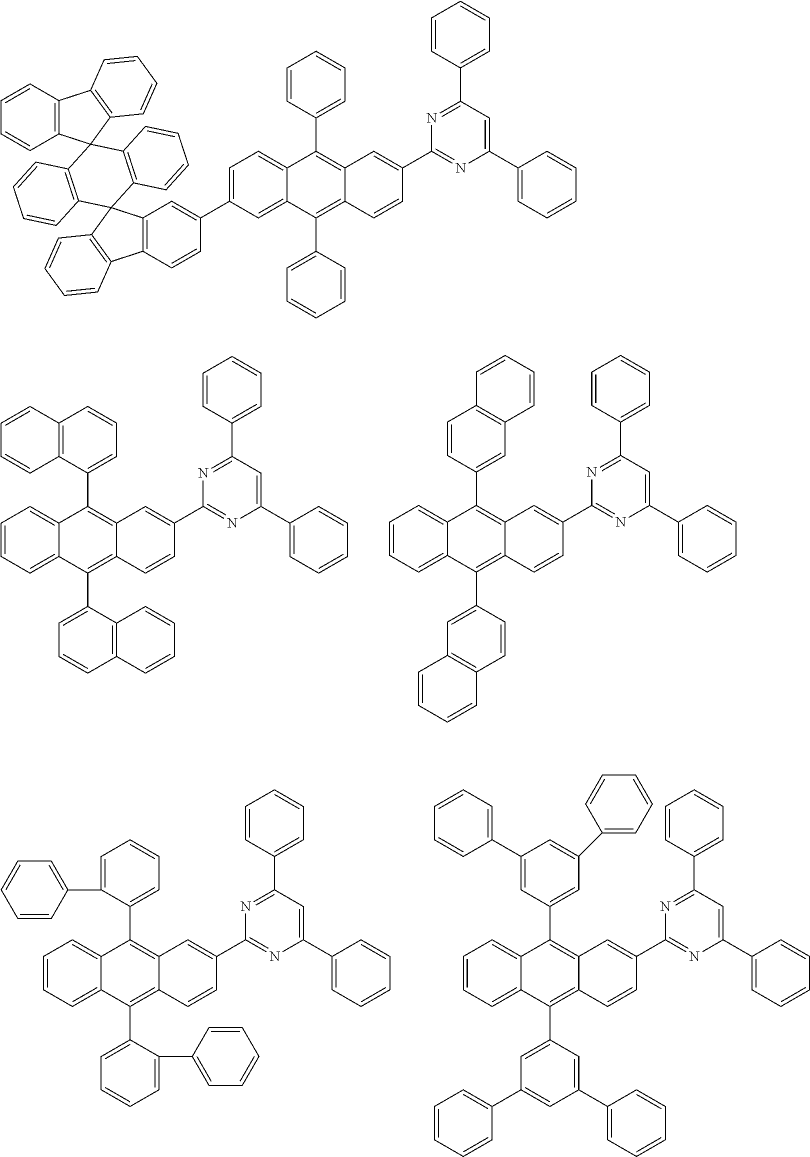

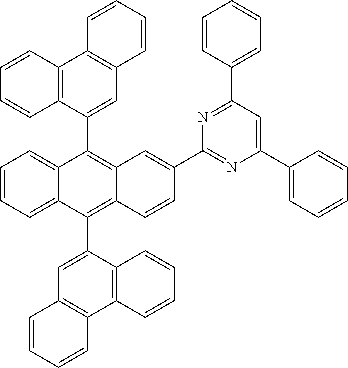 [SMILES: c1ccc(-c2cc(-c3ccccc3)nc(-c3ccc4c(-c5cc6ccccc6c6ccccc56)c5ccccc5c(-c5cc6ccccc6c6ccccc56)c4c3)n2)cc1]

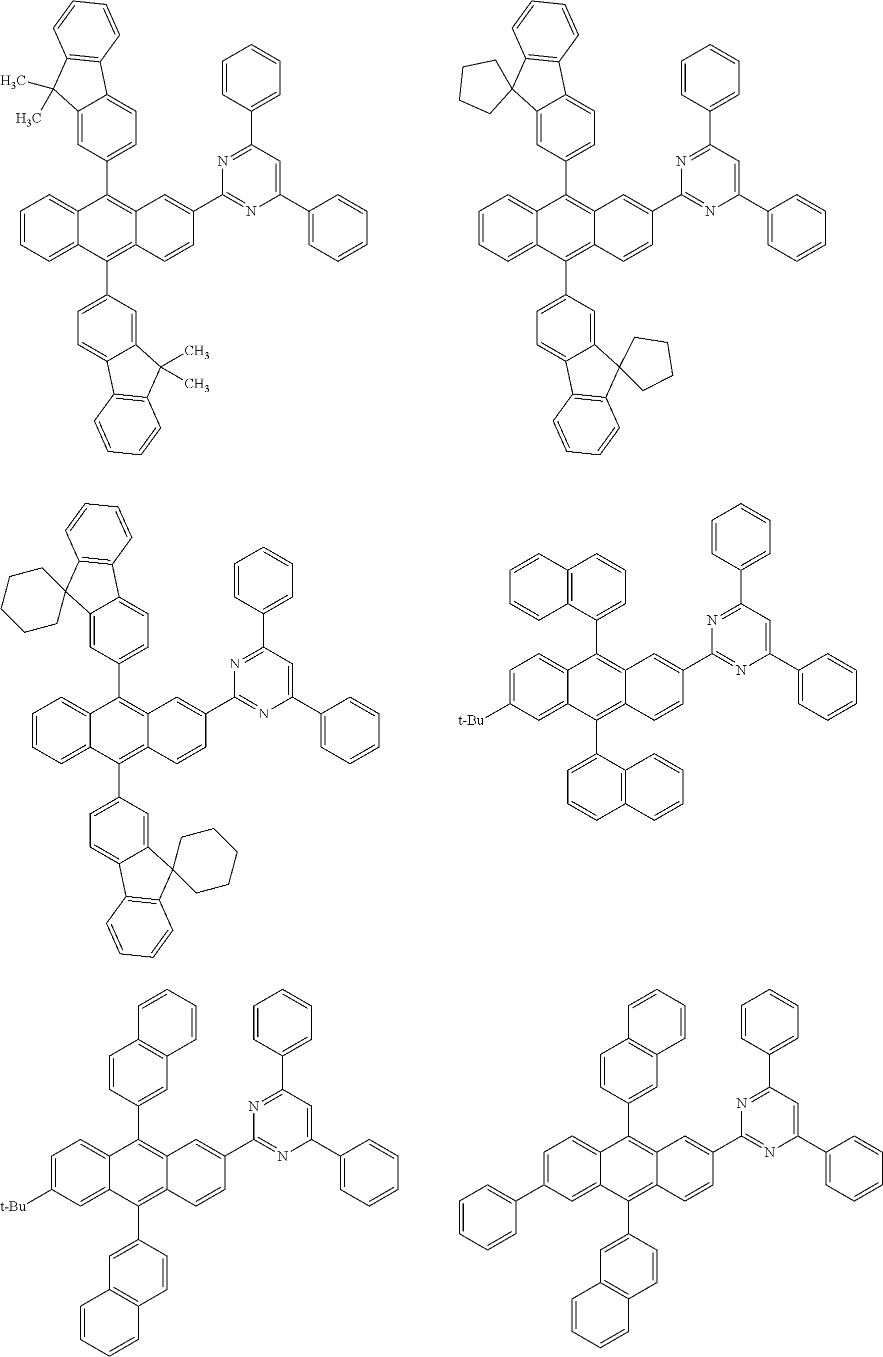

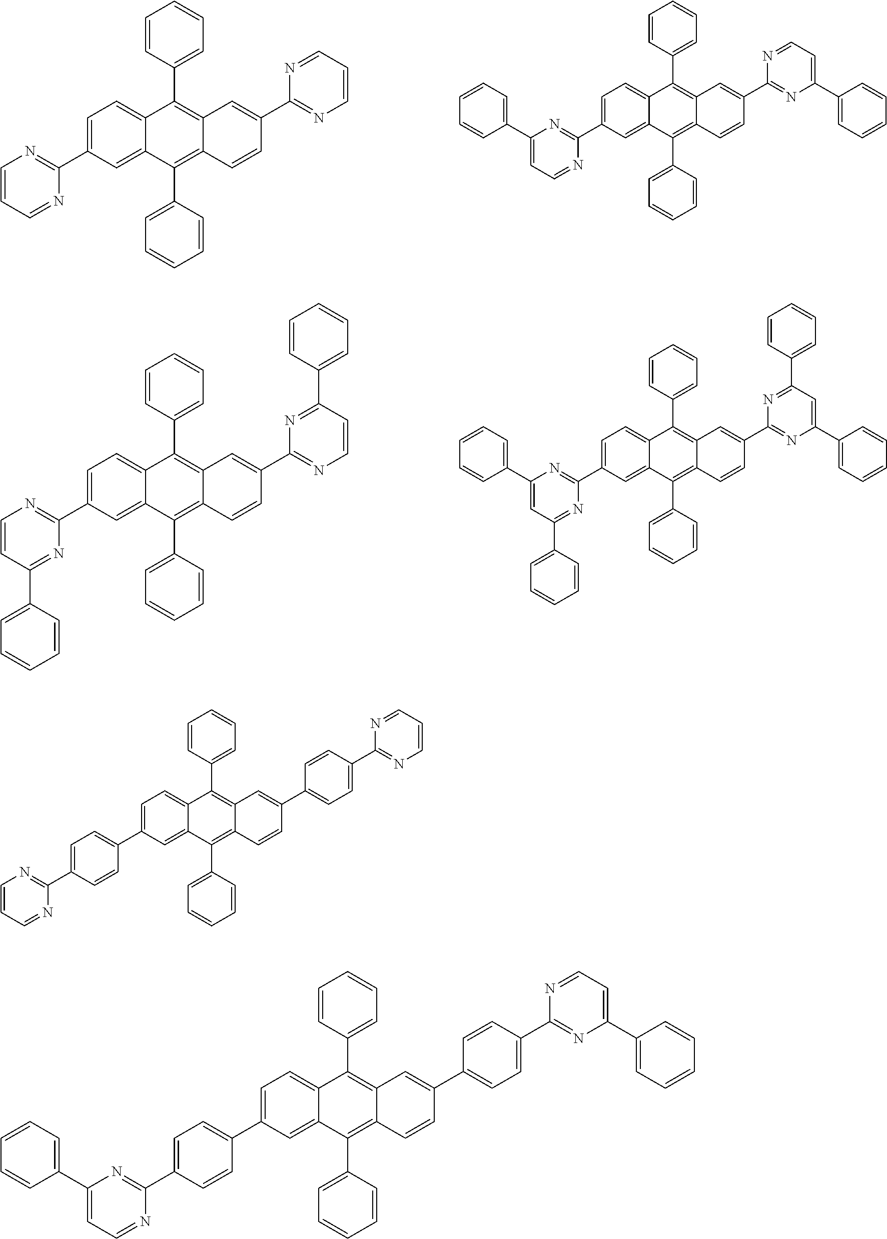

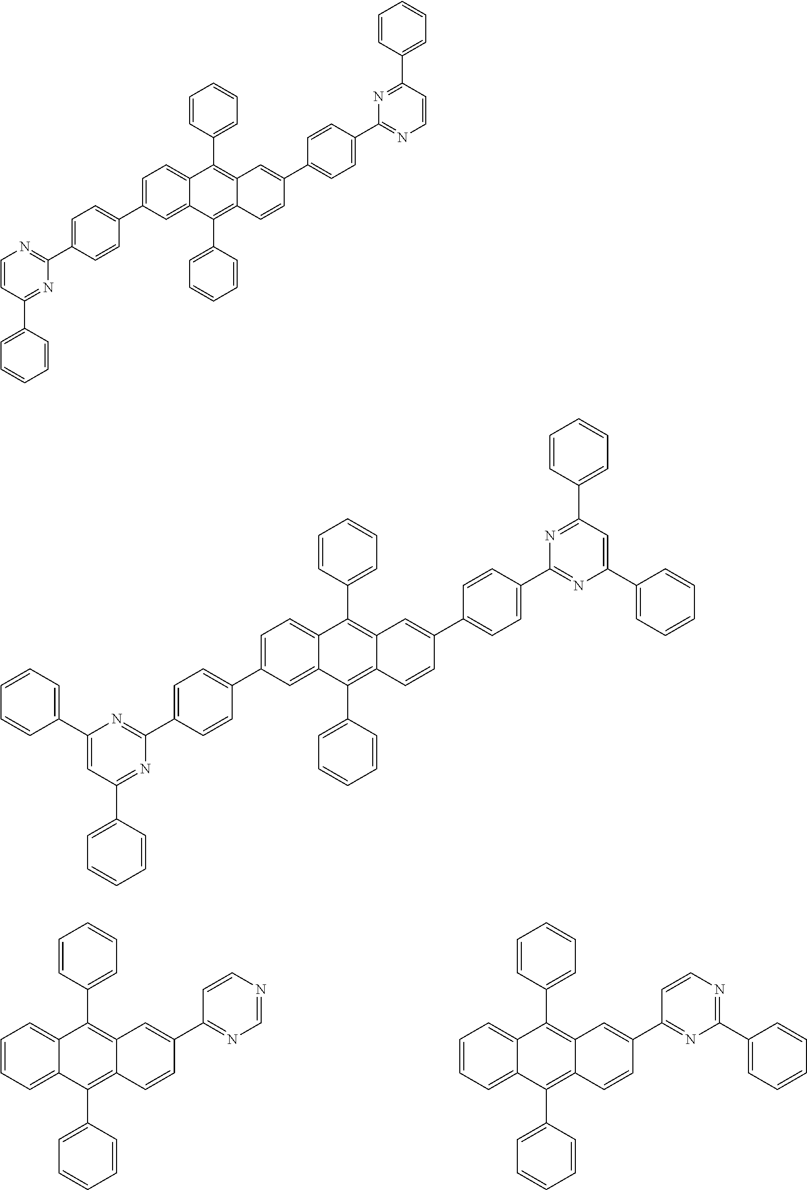 [SMILES: c1ccc(-c2ccnc(-c3ccc(-c4ccc5c(-c6ccccc6)c6cc(-c7ccc(-c8nccc(-c9ccccc9)n8)cc7)ccc6c(-c6ccccc6)c5c4)cc3)n2)cc1 ; c1ccc(-c2cc(-c3ccccc3)nc(-c3ccc(-c4ccc5c(-c6ccccc6)c6cc(-c7ccc(-c8nc(-c9ccccc9)cc(-c9ccccc9)n8)cc7)ccc6c(-c6ccccc6)c5c4)cc3)n2)cc1 ; c1ccc(-c2c3ccccc3c(-c3ccccc3)c3cc(-c4ccncn4)ccc23)cc1 ; c1ccc(-c2nccc(-c3ccc4c(-c5ccccc5)c5ccccc5c(-c5ccccc5)c4c3)n2)cc1]

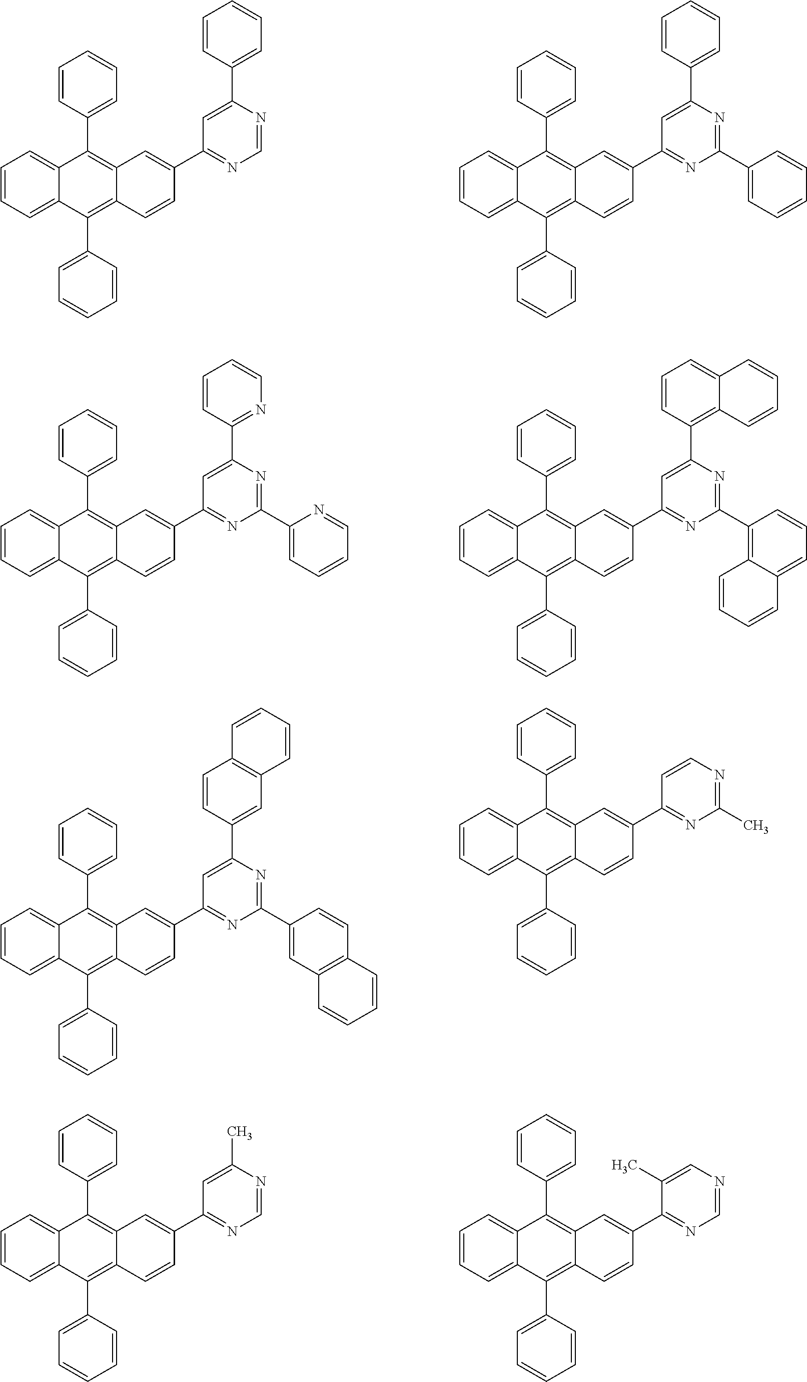

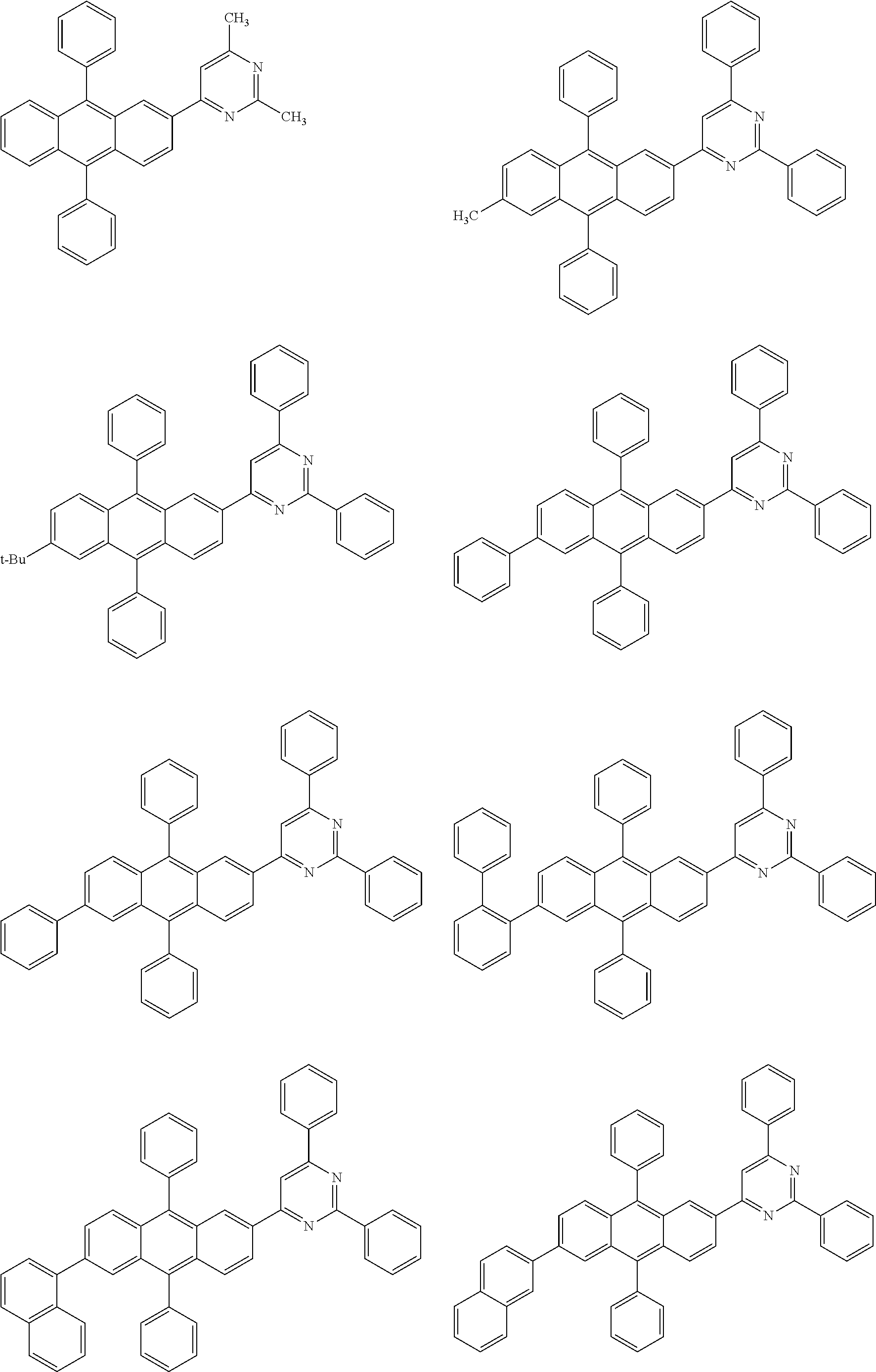

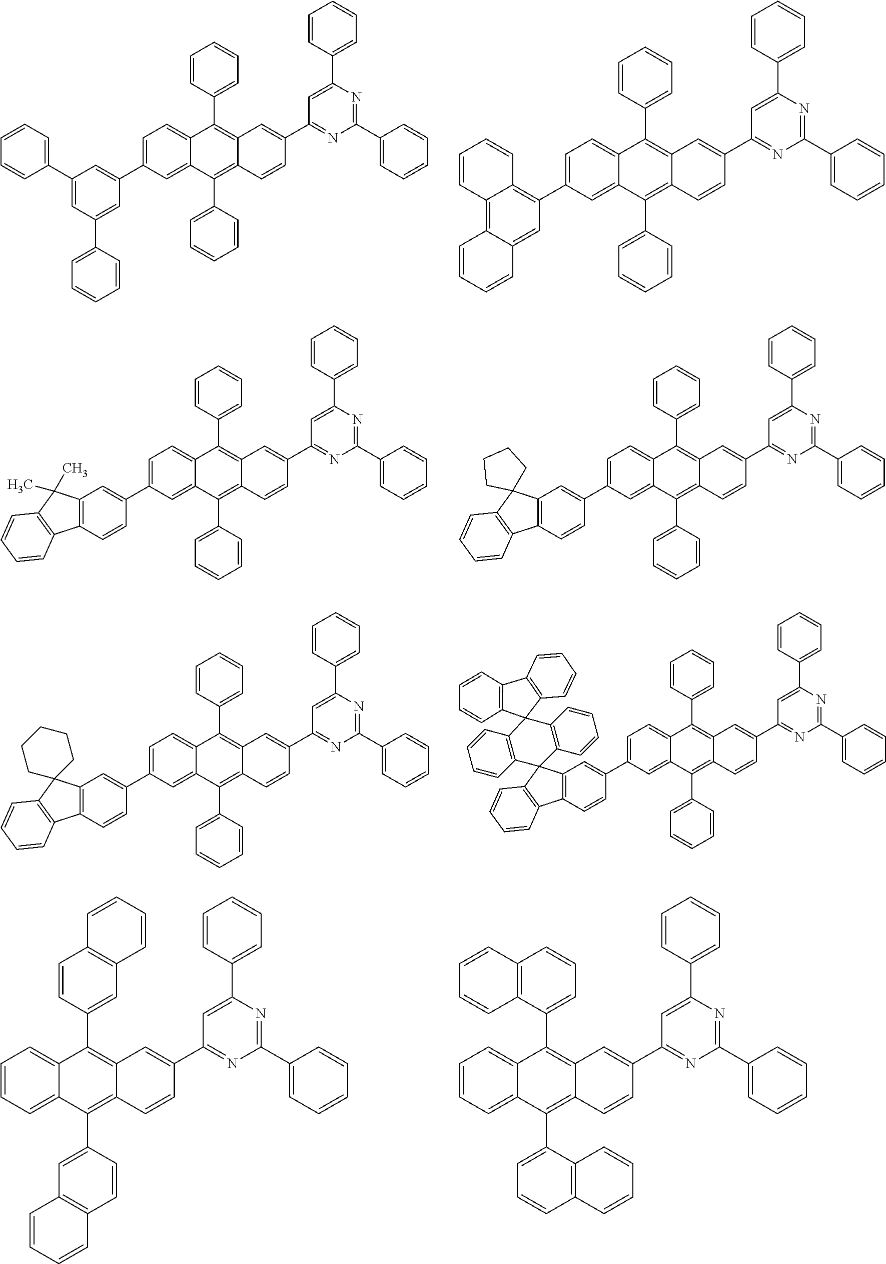

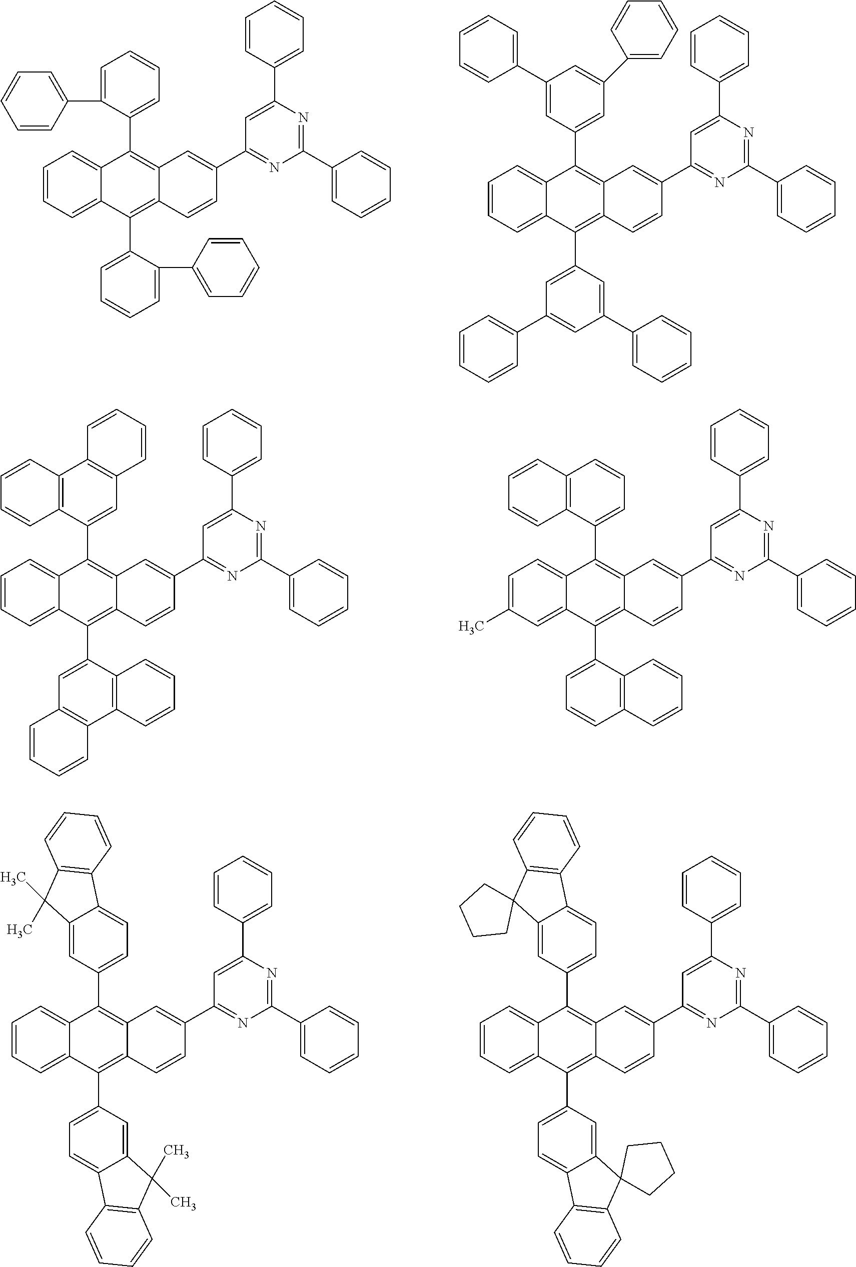

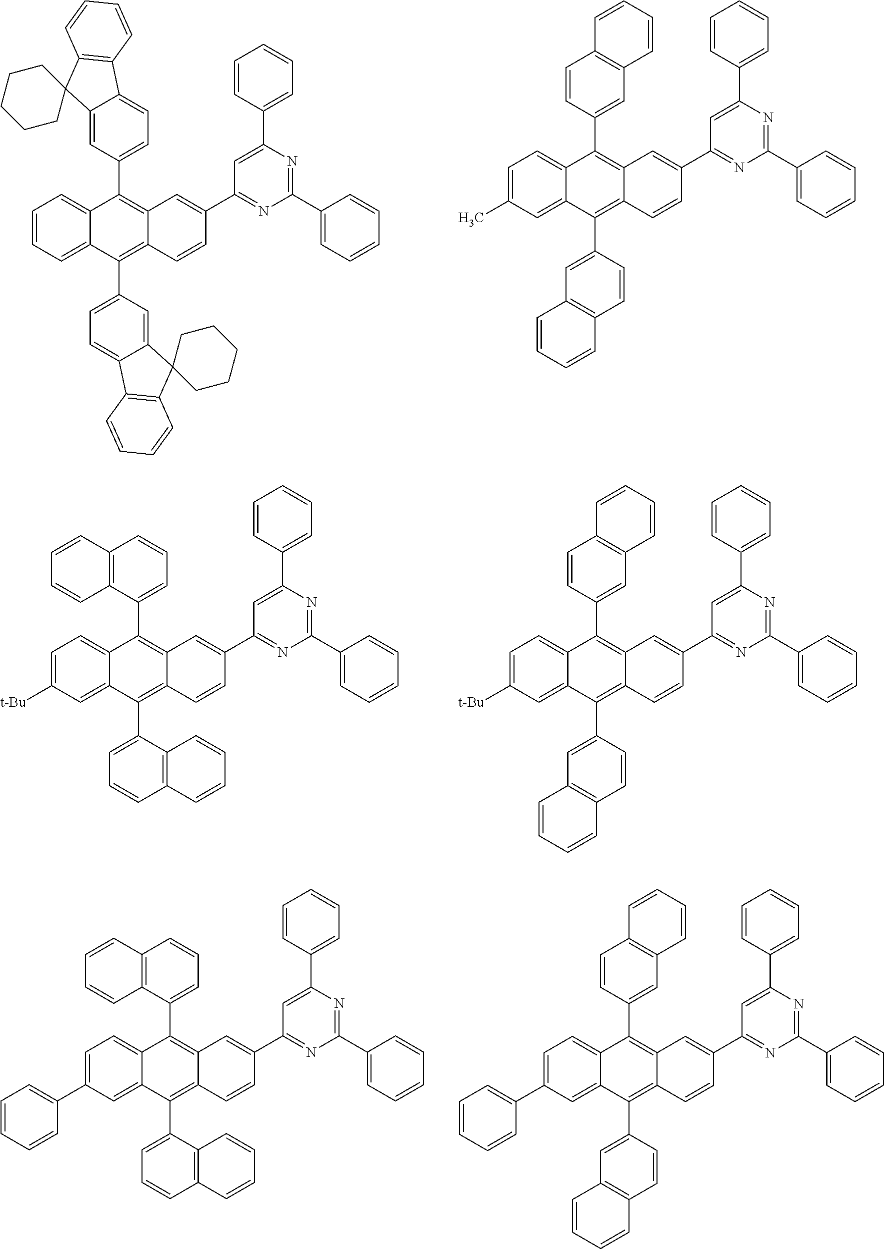

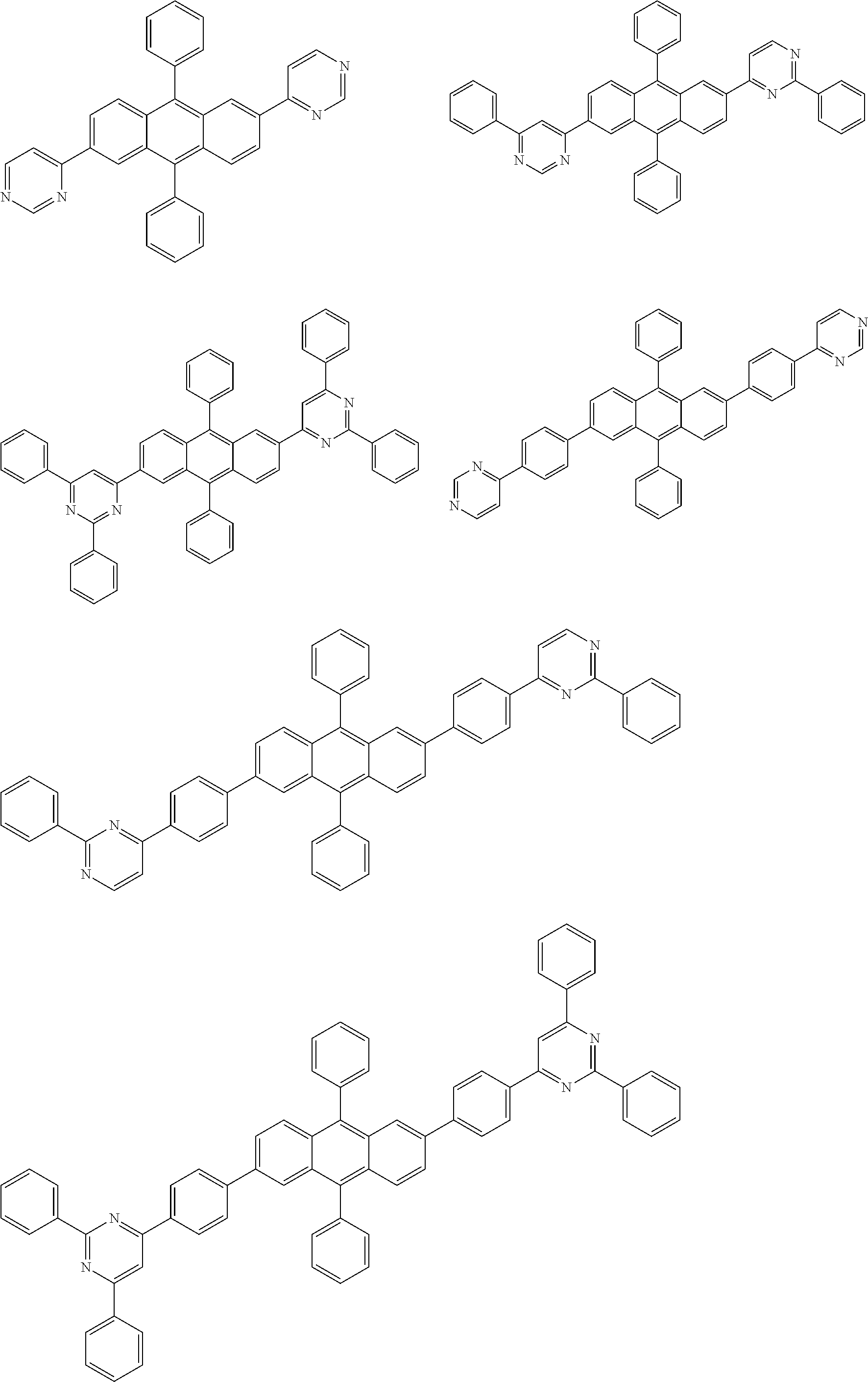

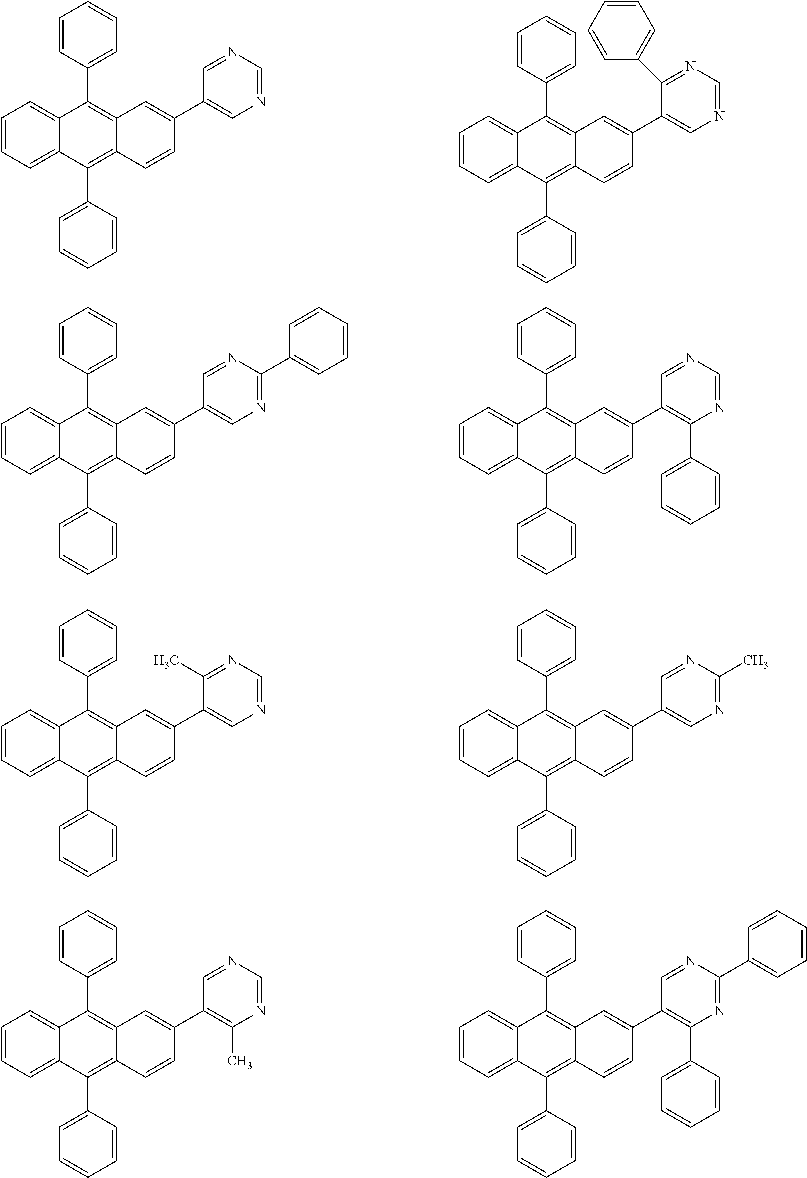

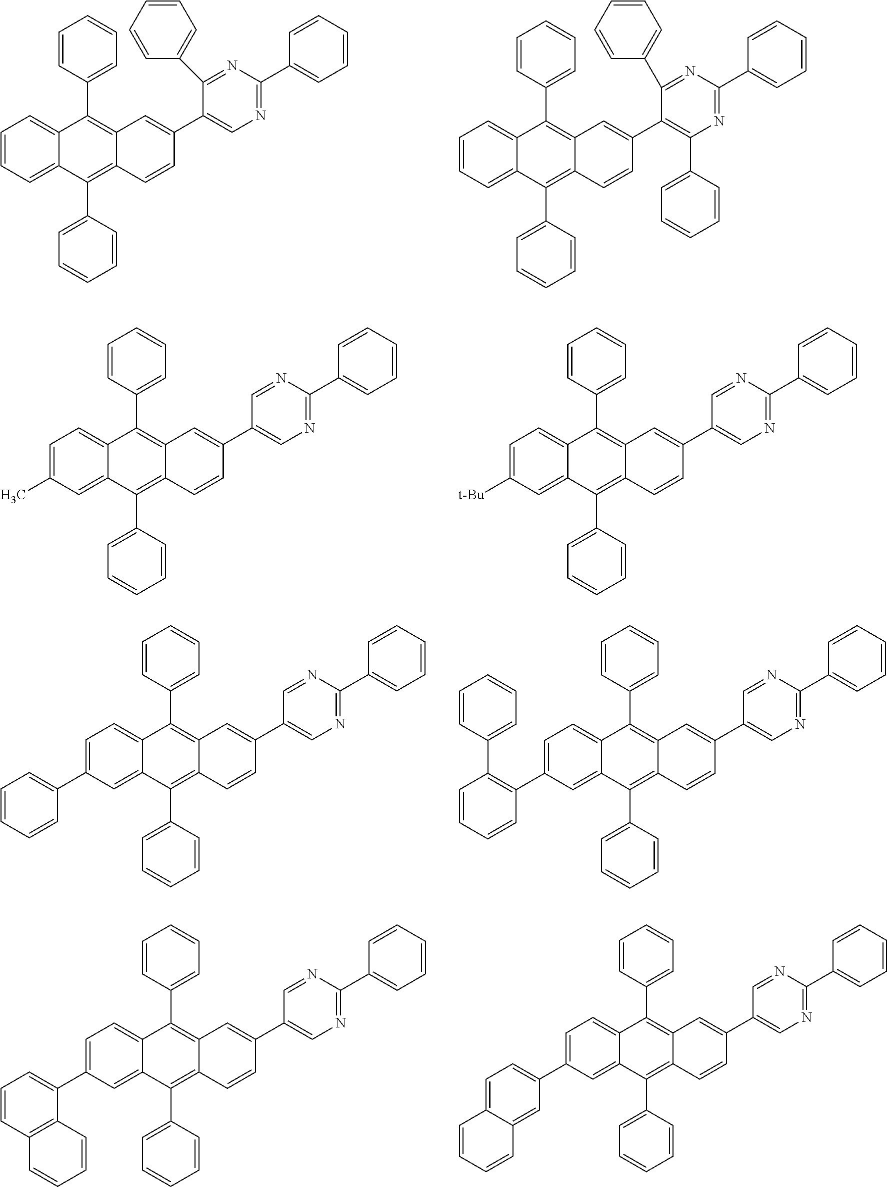

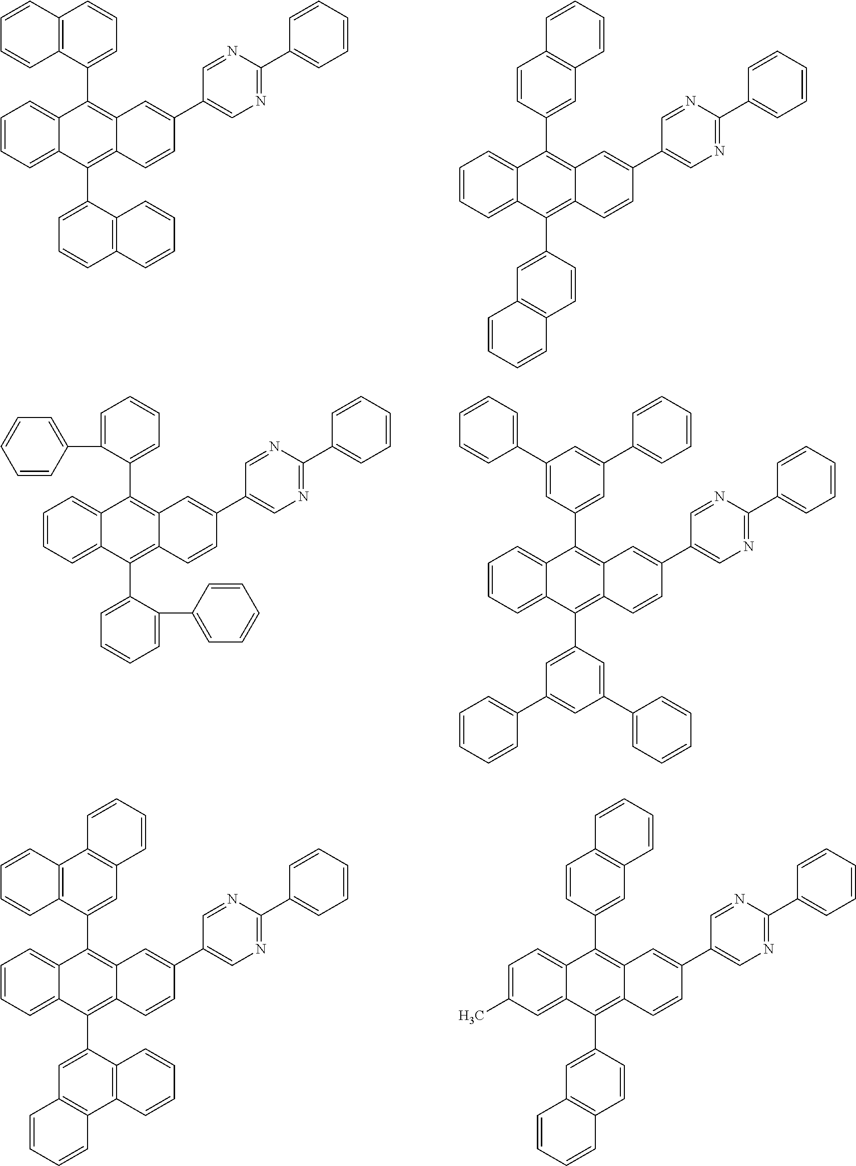

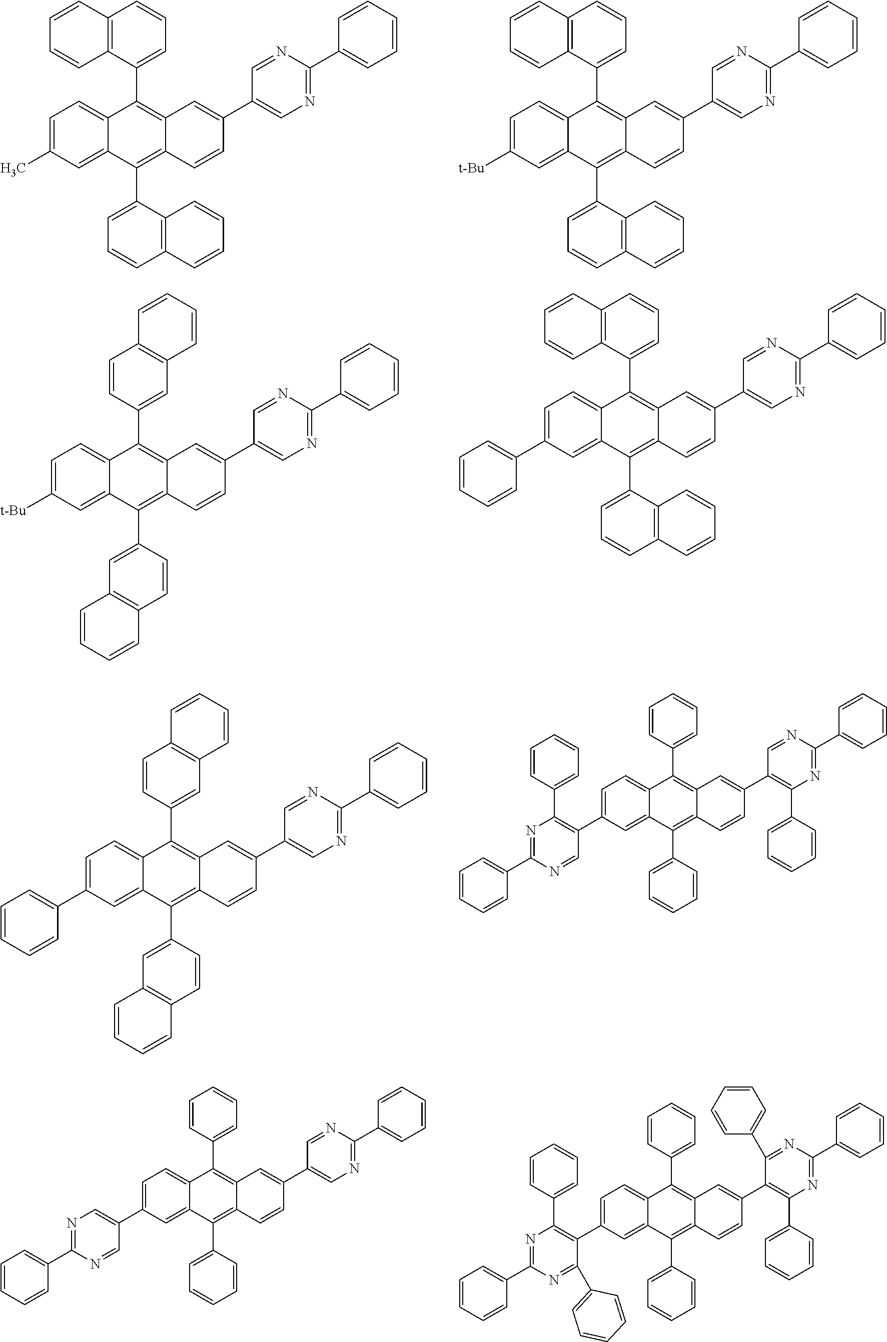

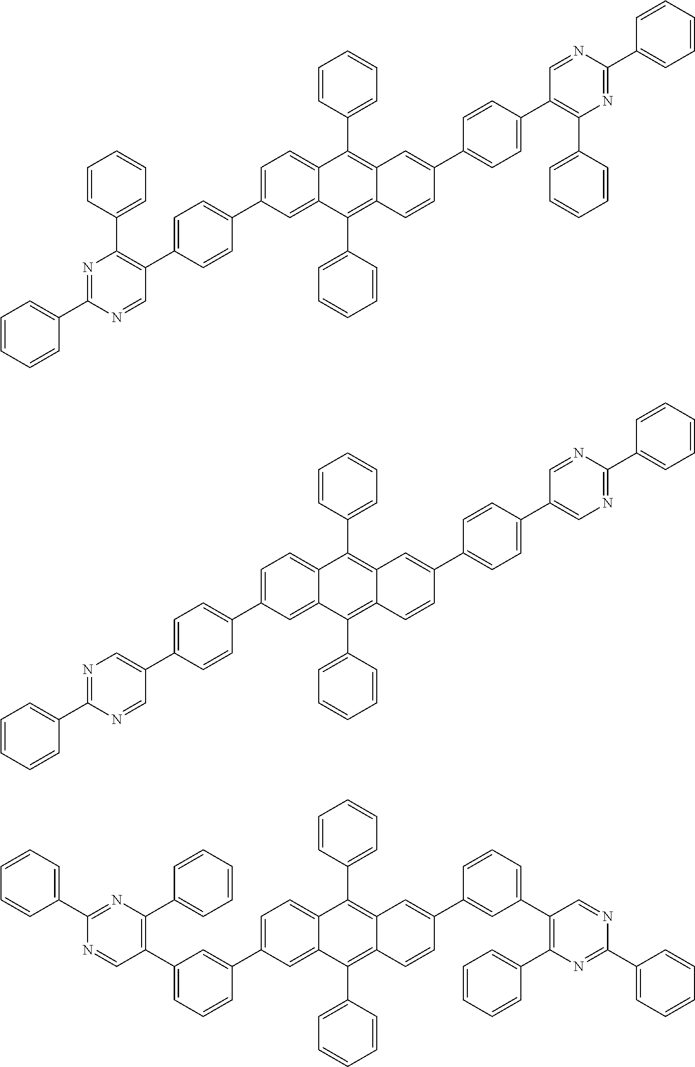 [SMILES: c1ccc(-c2ncc(-c3ccc(-c4ccc5c(-c6ccccc6)c6cc(-c7ccc(-c8cnc(-c9ccccc9)nc8-c8ccccc8)cc7)ccc6c(-c6ccccc6)c5c4)cc3)c(-c3ccccc3)n2)cc1 ; c1ccc(-c2ncc(-c3ccc(-c4ccc5c(-c6ccccc6)c6cc(-c7ccc(-c8cnc(-c9ccccc9)nc8)cc7)ccc6c(-c6ccccc6)c5c4)cc3)cn2)cc1 ; c1ccc(-c2ncc(-c3cccc(-c4ccc5c(-c6ccccc6)c6cc(-c7cccc(-c8cnc(-c9ccccc9)nc8-c8ccccc8)c7)ccc6c(-c6ccccc6)c5c4)c3)c(-c3ccccc3)n2)cc1]

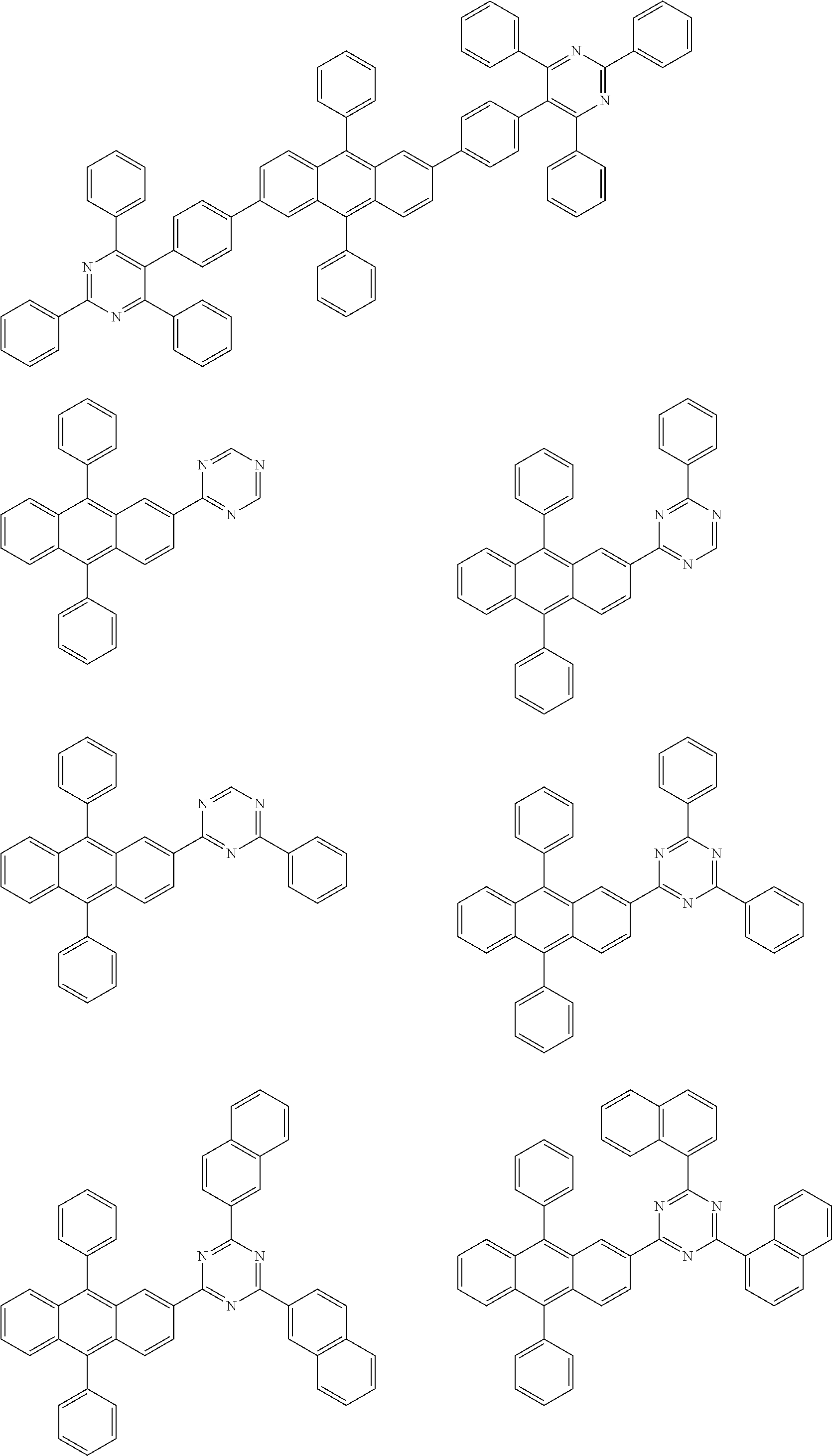

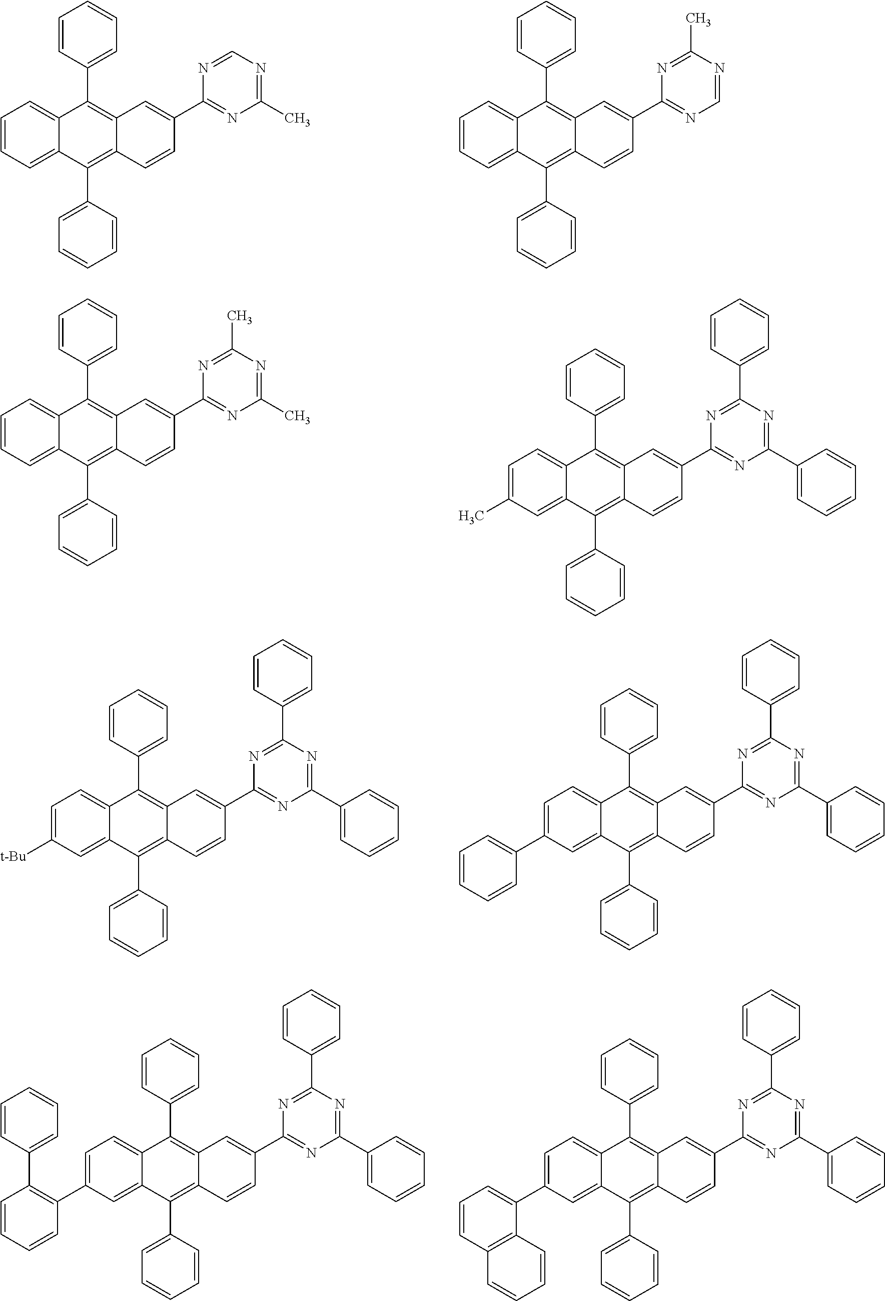

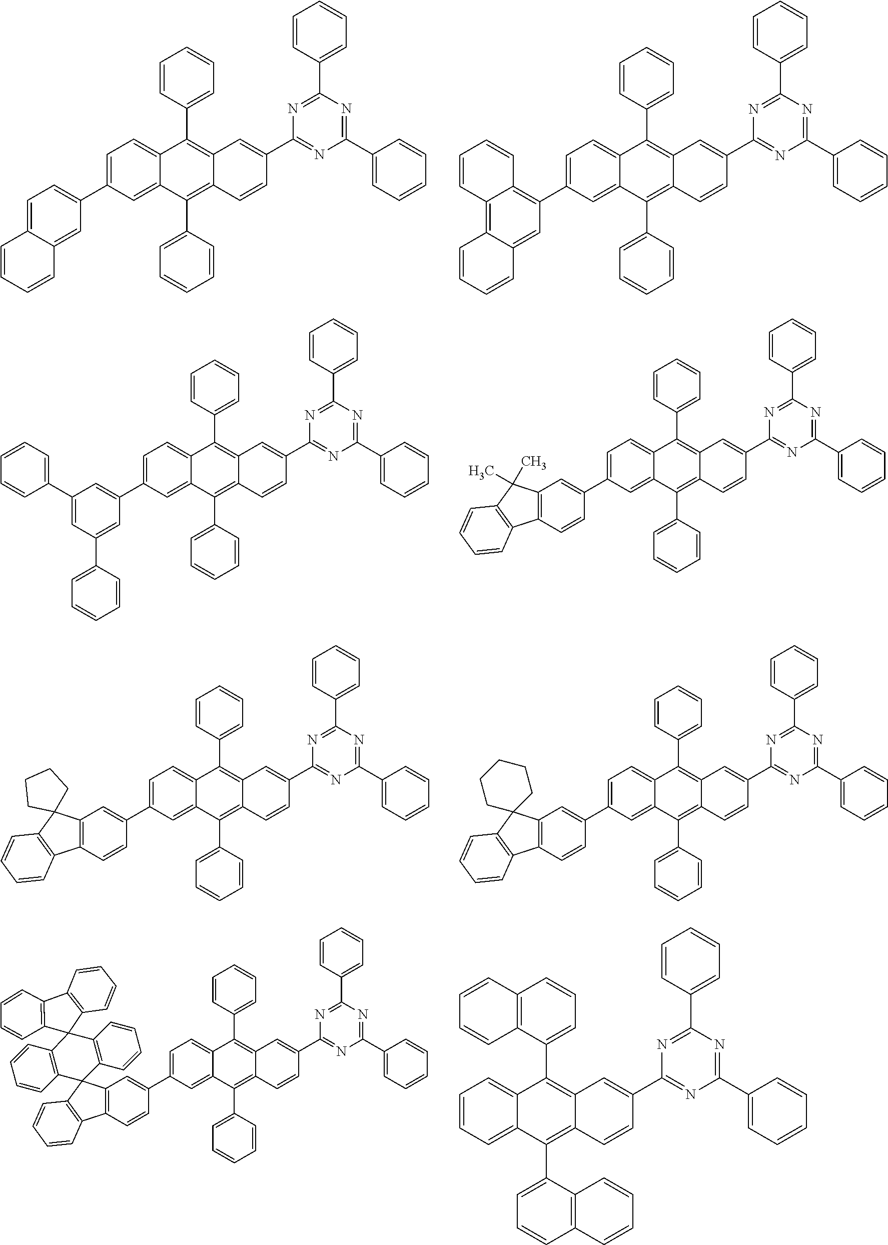

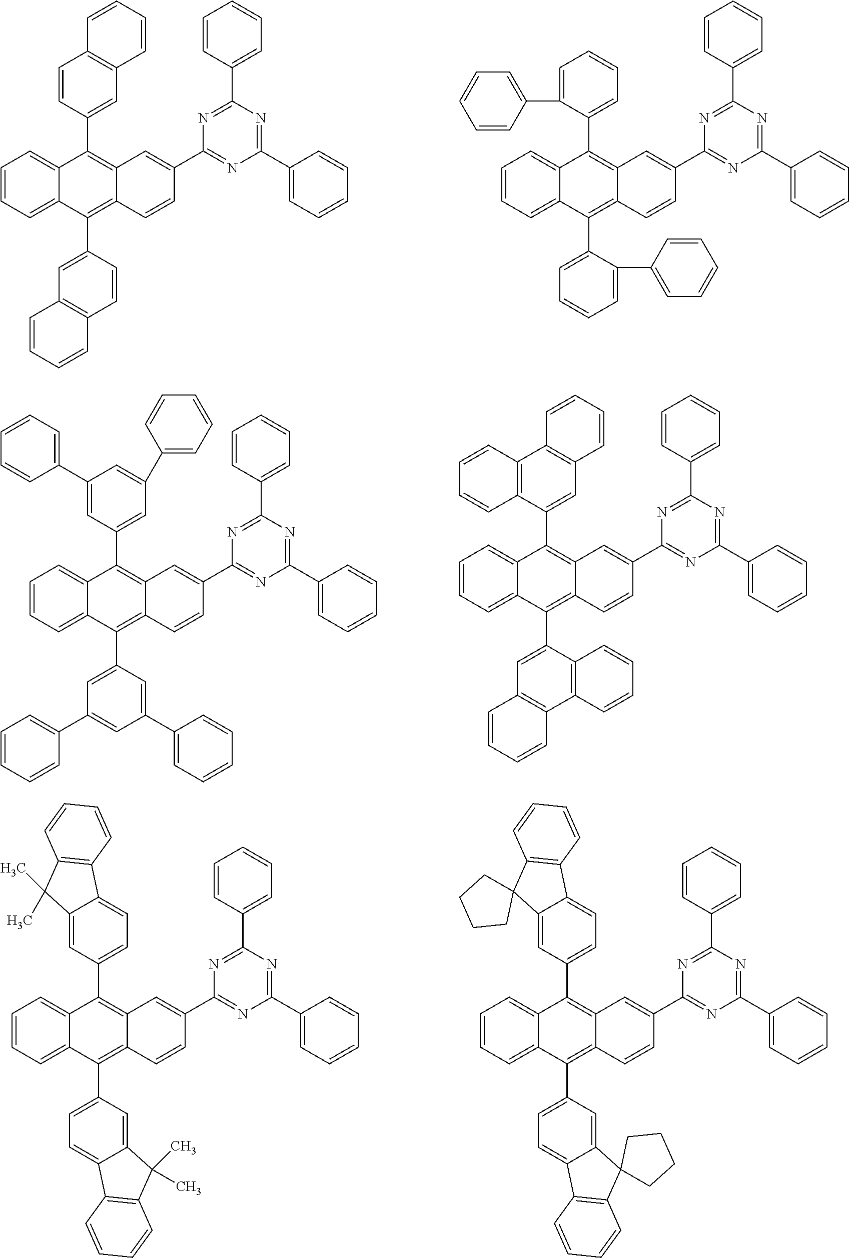

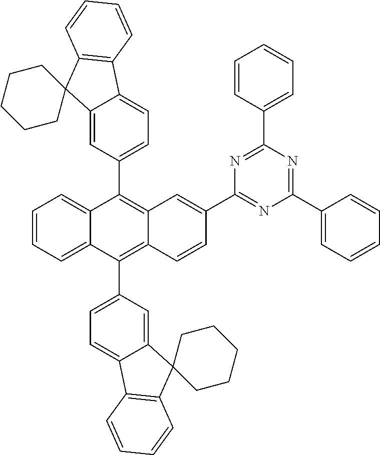 [SMILES: c1ccc(-c2nc(-c3ccccc3)nc(-c3ccc4c(-c5ccc6c(c5)C5(CCCCC5)c5ccccc5-6)c5ccccc5c(-c5ccc6c(c5)C5(CCCCC5)c5ccccc5-6)c4c3)n2)cc1]

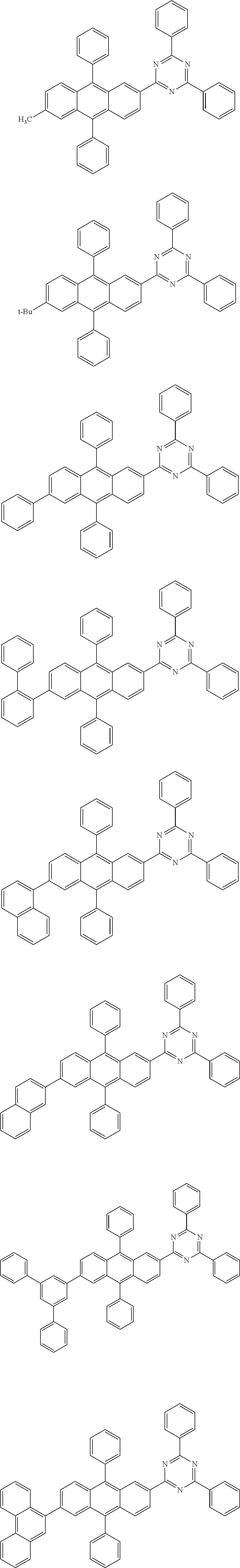

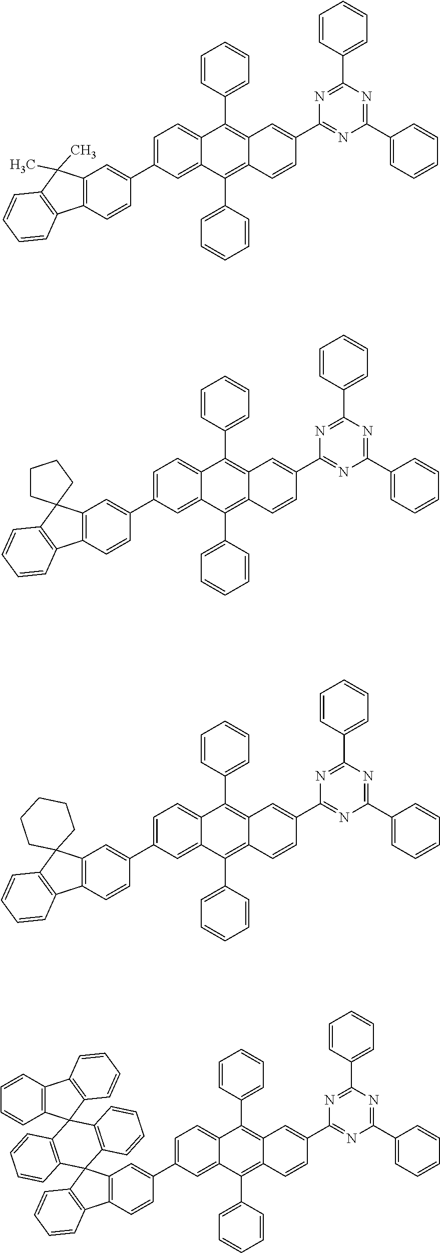 [SMILES: CC1(C)c2ccccc2-c2ccc(-c3ccc4c(-c5ccccc5)c5cc(-c6nc(-c7ccccc7)nc(-c7ccccc7)n6)ccc5c(-c5ccccc5)c4c3)cc21 ; c1ccc(-c2nc(-c3ccccc3)nc(-c3ccc4c(-c5ccccc5)c5cc(-c6ccc7c(c6)C6(CCCC6)c6ccccc6-7)ccc5c(-c5ccccc5)c4c3)n2)cc1 ; c1ccc(-c2nc(-c3ccccc3)nc(-c3ccc4c(-c5ccccc5)c5cc(-c6ccc7c(c6)C6(CCCCC6)c6ccccc6-7)ccc5c(-c5ccccc5)c4c3)n2)cc1 ; c1ccc(-c2nc(-c3ccccc3)nc(-c3ccc4c(-c5ccccc5)c5cc(-c6ccc7c(c6)C6(c8ccccc8-7)c7ccccc7C7(c8ccccc8-c8ccccc87)c7ccccc76)ccc5c(-c5ccccc5)c4c3)n2)cc1]

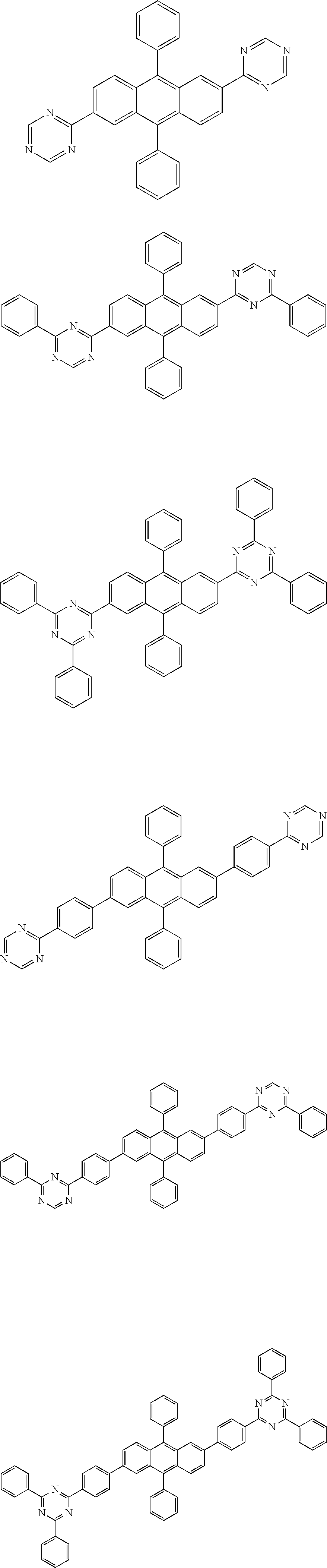

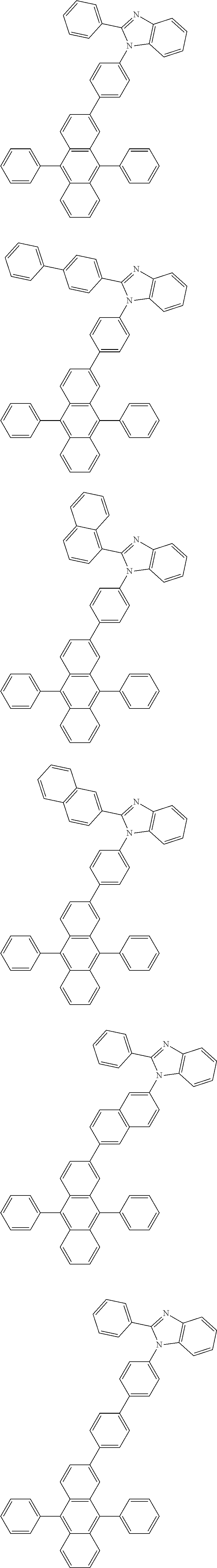

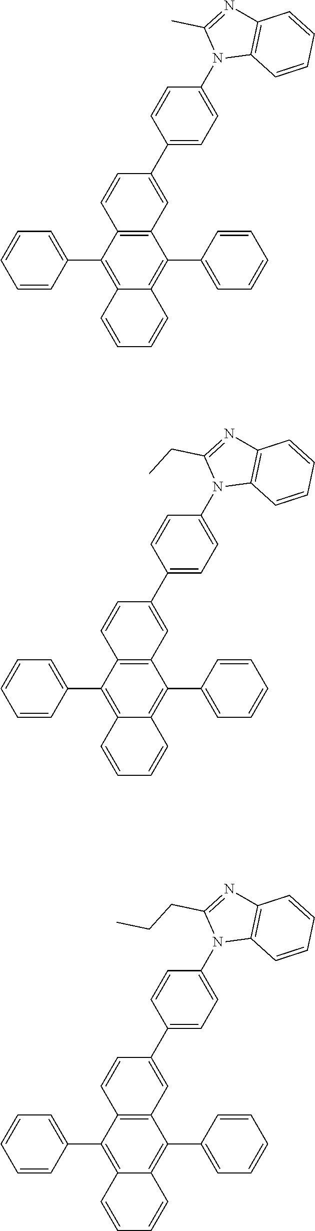

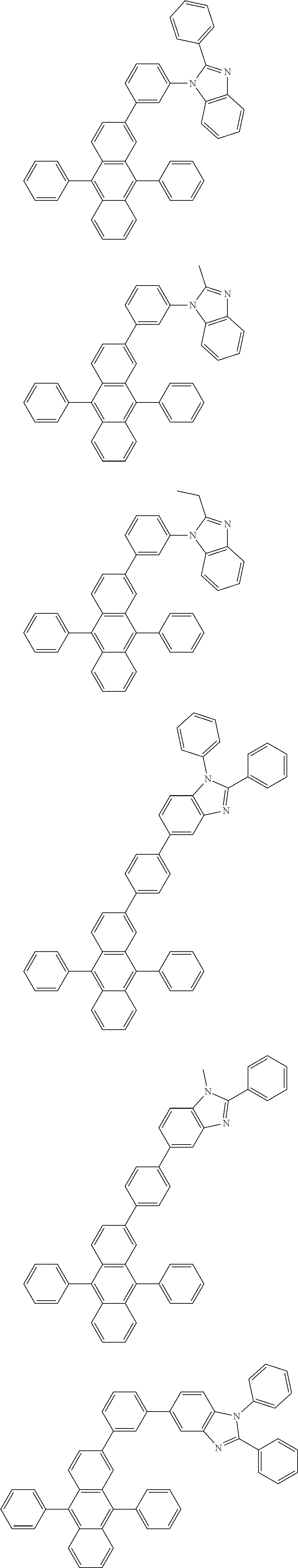 [SMILES: c1ccc(-c2c3ccccc3c(-c3ccccc3)c3cc(-c4cccc(-n5c(-c6ccccc6)nc6ccccc65)c4)ccc23)cc1 ; Cc1nc2ccccc2n1-c1cccc(-c2ccc3c(-c4ccccc4)c4ccccc4c(-c4ccccc4)c3c2)c1 ; CCc1nc2ccccc2n1-c1cccc(-c2ccc3c(-c4ccccc4)c4ccccc4c(-c4ccccc4)c3c2)c1 ; c1ccc(-c2c3ccccc3c(-c3ccccc3)c3cc(-c4ccc(-c5ccc6c(c5)nc(-c5ccccc5)n6-c5ccccc5)cc4)ccc23)cc1 ; Cn1c(-c2ccccc2)nc2cc(-c3ccc(-c4ccc5c(-c6ccccc6)c6ccccc6c(-c6ccccc6)c5c4)cc3)ccc21 ; c1ccc(-c2c3ccccc3c(-c3ccccc3)c3cc(-c4cccc(-c5ccc6c(c5)nc(-c5ccccc5)n6-c5ccccc5)c4)ccc23)cc1]

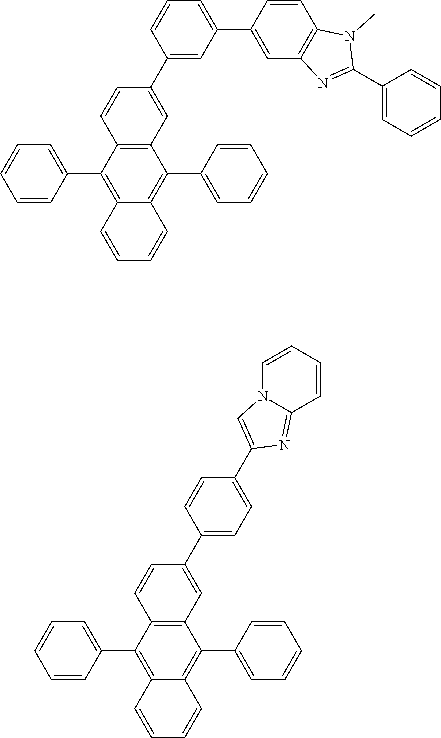 [SMILES: Cn1c(-c2ccccc2)nc2cc(-c3cccc(-c4ccc5c(-c6ccccc6)c6ccccc6c(-c6ccccc6)c5c4)c3)ccc21 ; c1ccc(-c2c3ccccc3c(-c3ccccc3)c3cc(-c4ccc(-c5cn6ccccc6n5)cc4)ccc23)cc1]

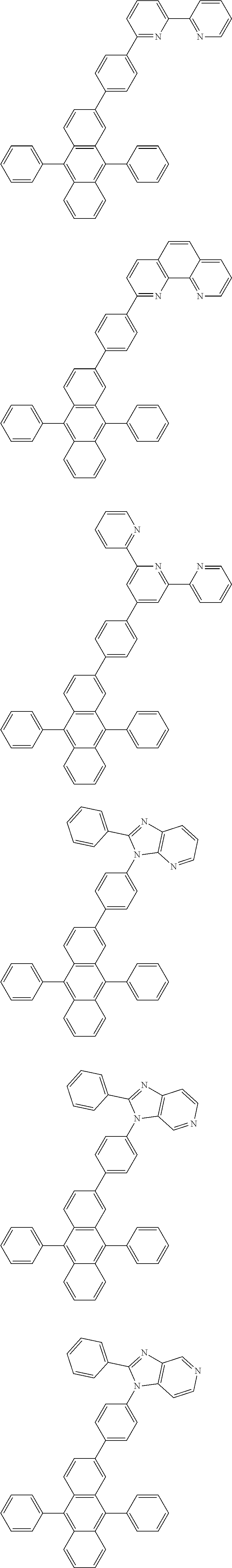

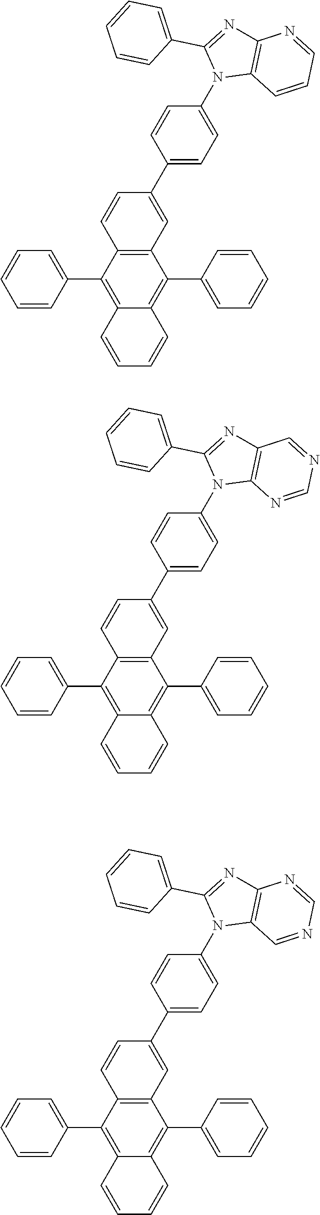

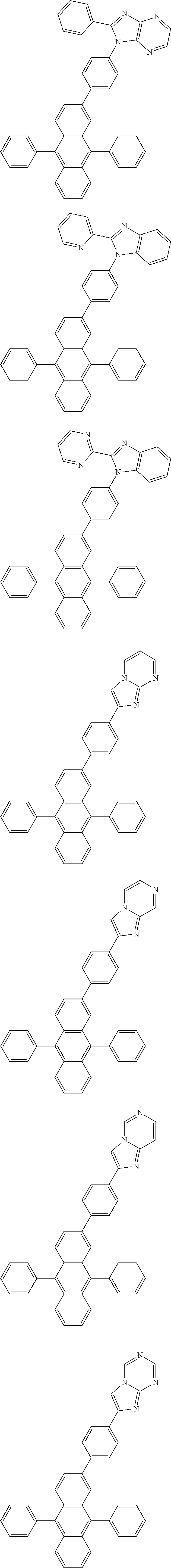

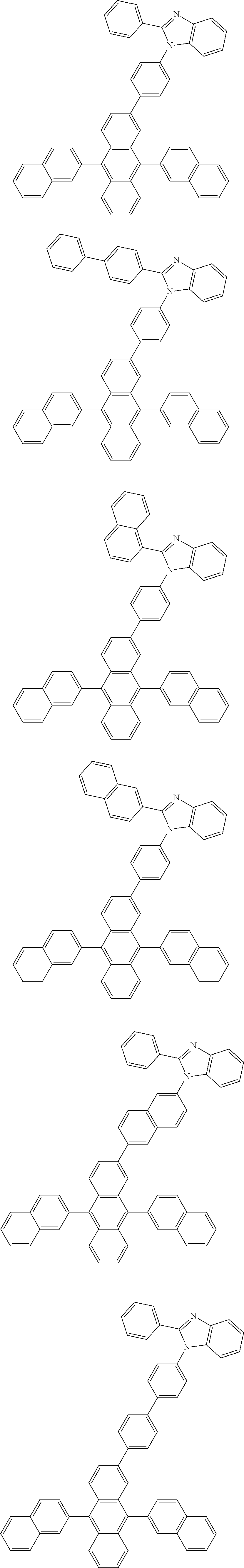

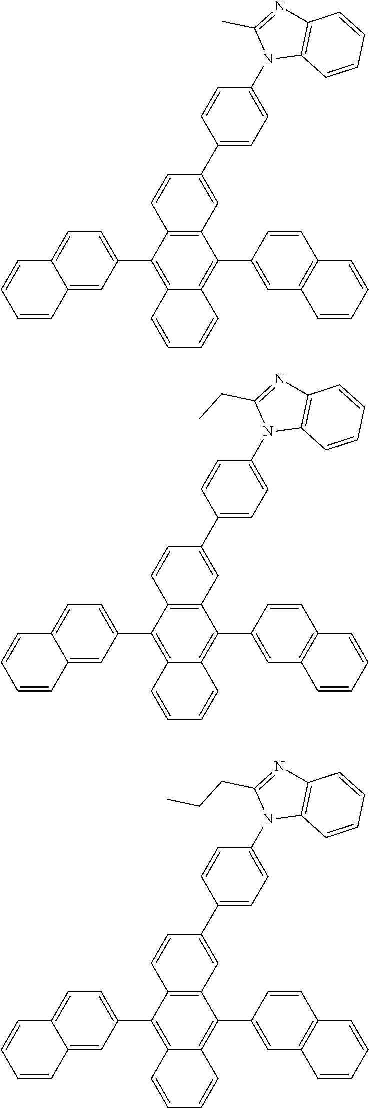 [SMILES: Cc1nc2ccccc2n1-c1ccc(-c2ccc3c(-c4ccc5ccccc5c4)c4ccccc4c(-c4ccc5ccccc5c4)c3c2)cc1 ; CCc1nc2ccccc2n1-c1ccc(-c2ccc3c(-c4ccc5ccccc5c4)c4ccccc4c(-c4ccc5ccccc5c4)c3c2)cc1 ; CCCc1nc2ccccc2n1-c1ccc(-c2ccc3c(-c4ccc5ccccc5c4)c4ccccc4c(-c4ccc5ccccc5c4)c3c2)cc1]

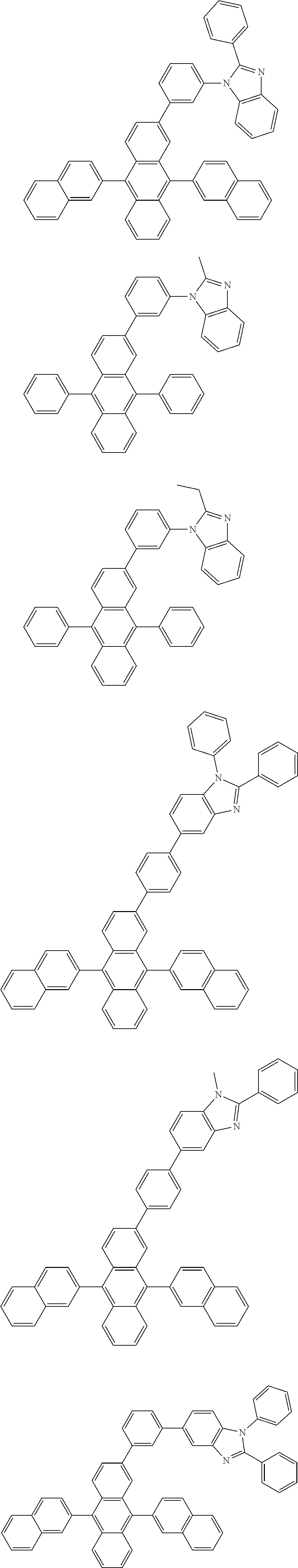

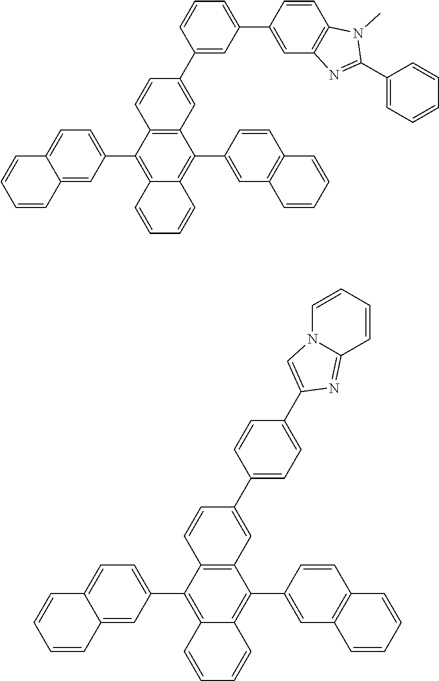 [SMILES: Cn1c(-c2ccccc2)nc2cc(-c3cccc(-c4ccc5c(-c6ccc7ccccc7c6)c6ccccc6c(-c6ccc7ccccc7c6)c5c4)c3)ccc21 ; c1ccc2cc(-c3c4ccccc4c(-c4ccc5ccccc5c4)c4cc(-c5ccc(-c6cn7ccccc7n6)cc5)ccc34)ccc2c1]

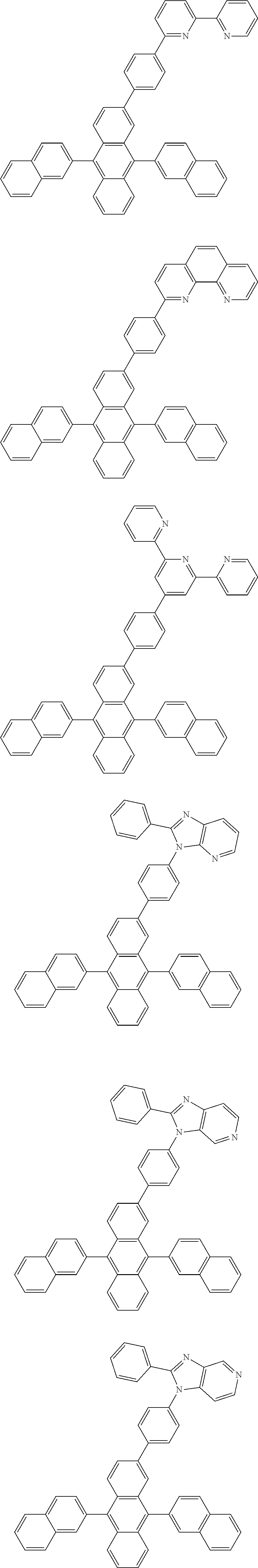

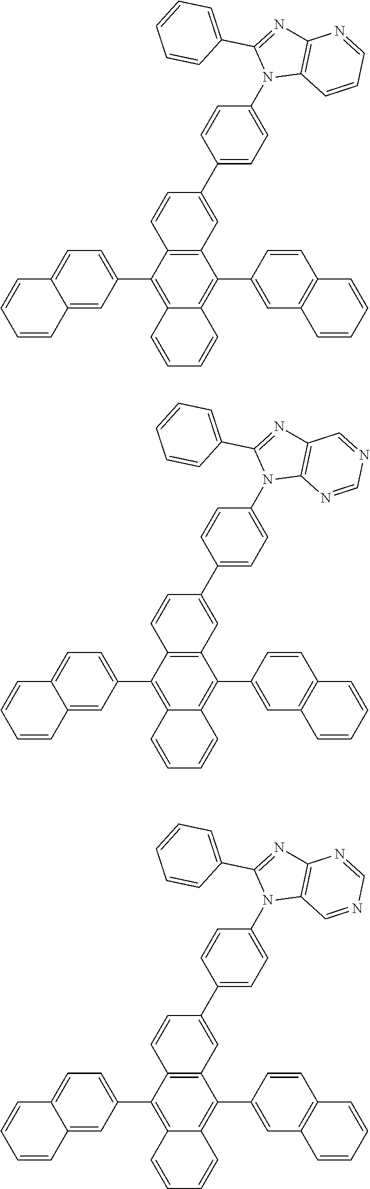

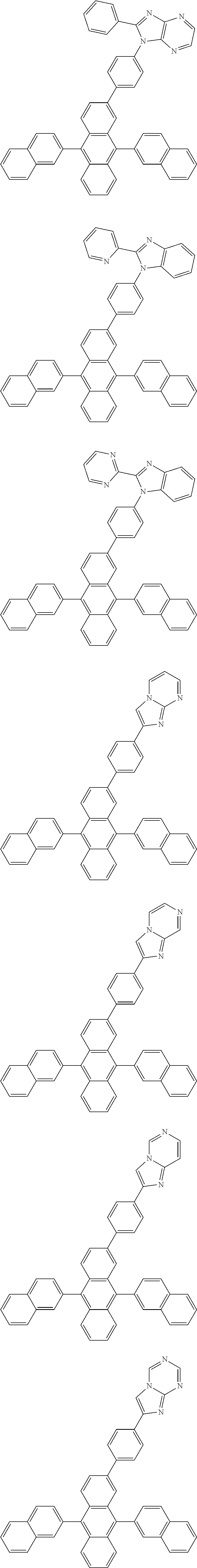

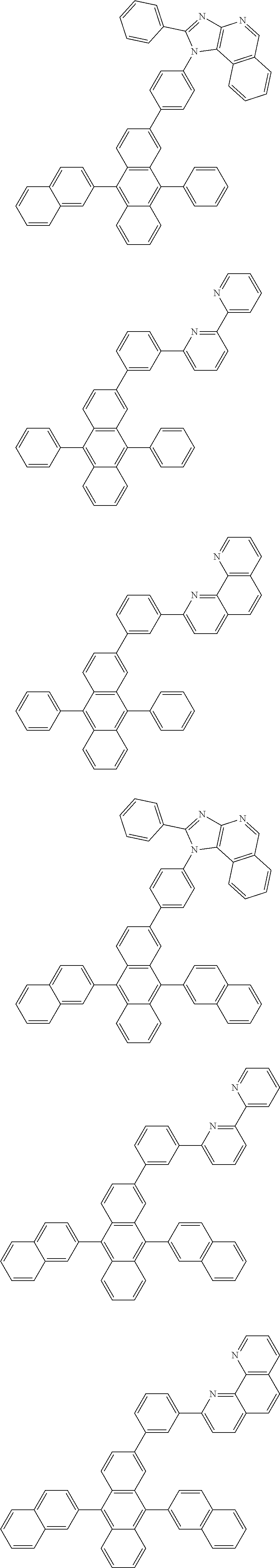

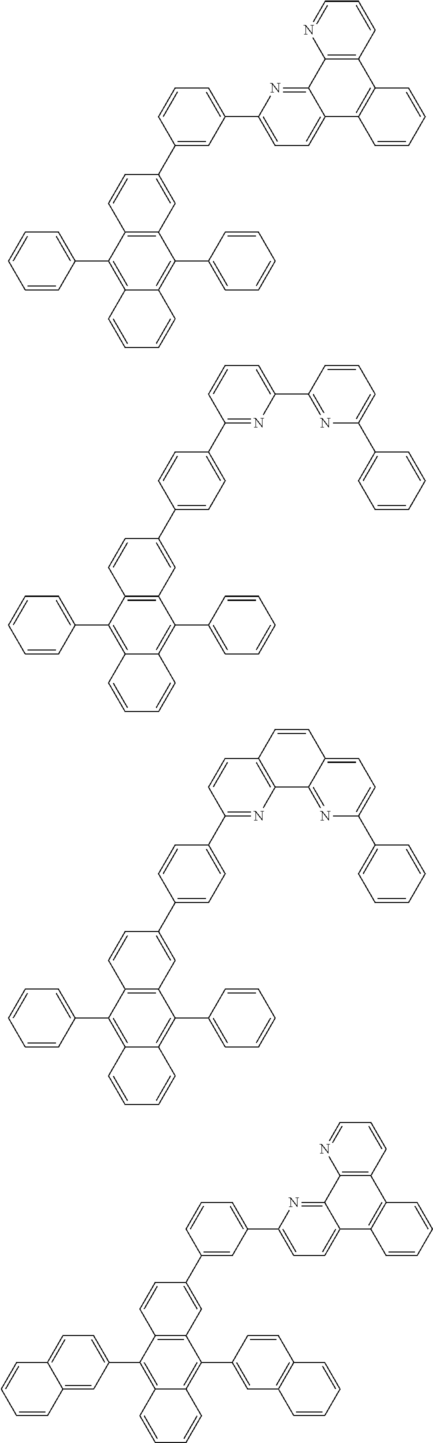 [SMILES: c1ccc(-c2c3ccccc3c(-c3ccccc3)c3cc(-c4cccc(-c5ccc6c7ccccc7c7cccnc7c6n5)c4)ccc23)cc1 ; c1ccc(-c2cccc(-c3cccc(-c4ccc(-c5ccc6c(-c7ccccc7)c7ccccc7c(-c7ccccc7)c6c5)cc4)n3)n2)cc1 ; c1ccc(-c2ccc3ccc4ccc(-c5ccc(-c6ccc7c(-c8ccccc8)c8ccccc8c(-c8ccccc8)c7c6)cc5)nc4c3n2)cc1 ; c1cc(-c2ccc3c(-c4ccc5ccccc5c4)c4ccccc4c(-c4ccc5ccccc5c4)c3c2)cc(-c2ccc3c4ccccc4c4cccnc4c3n2)c1]

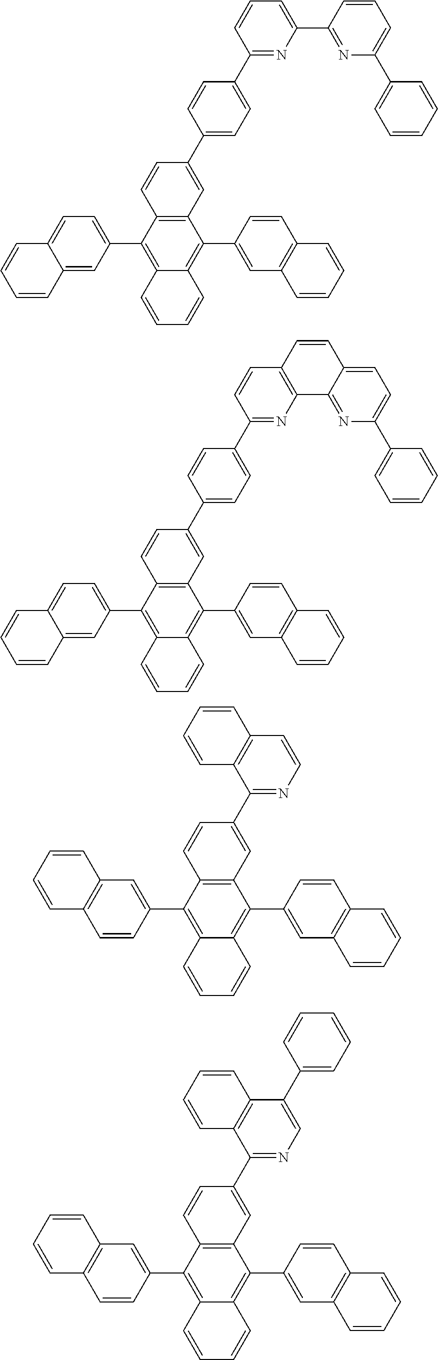

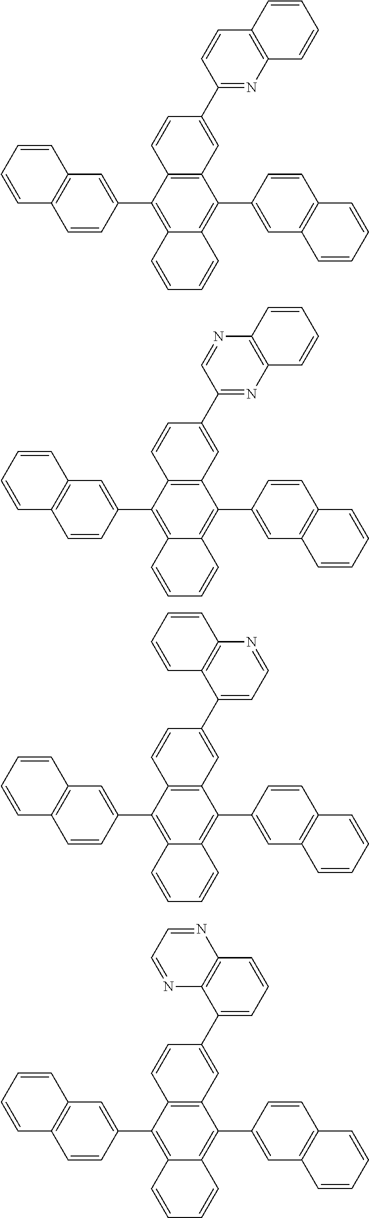

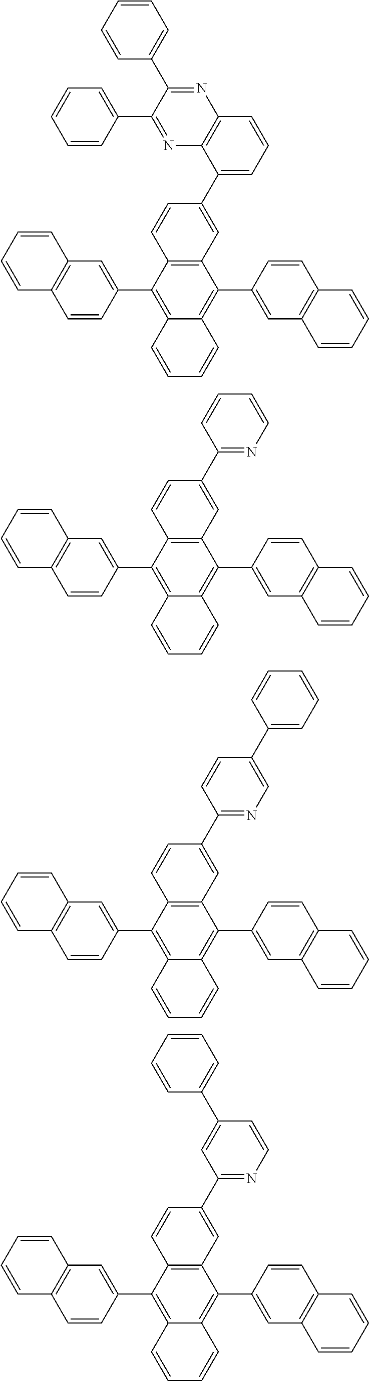

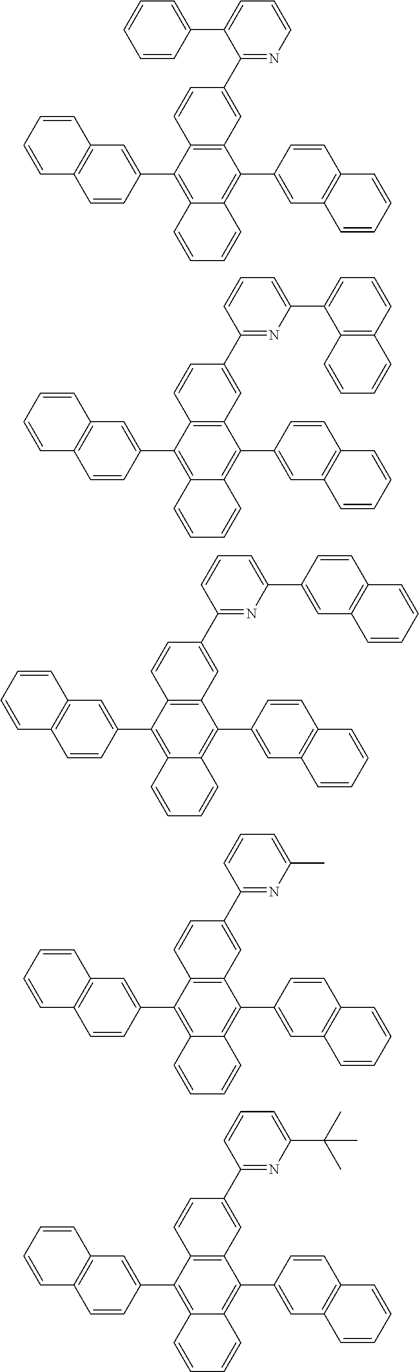

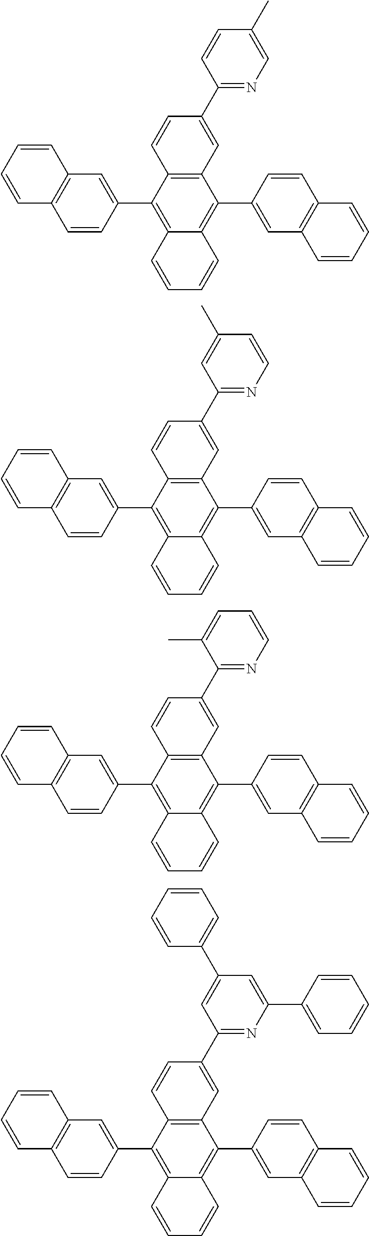

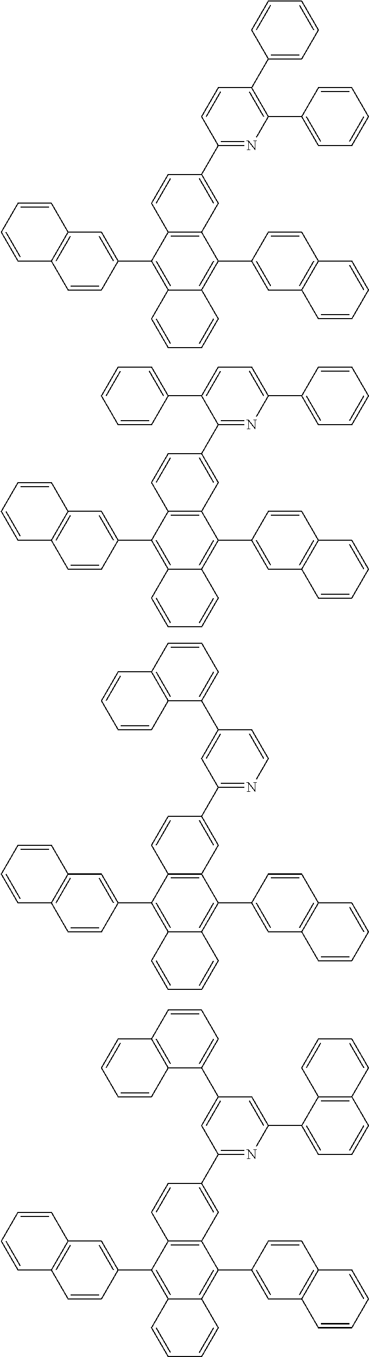

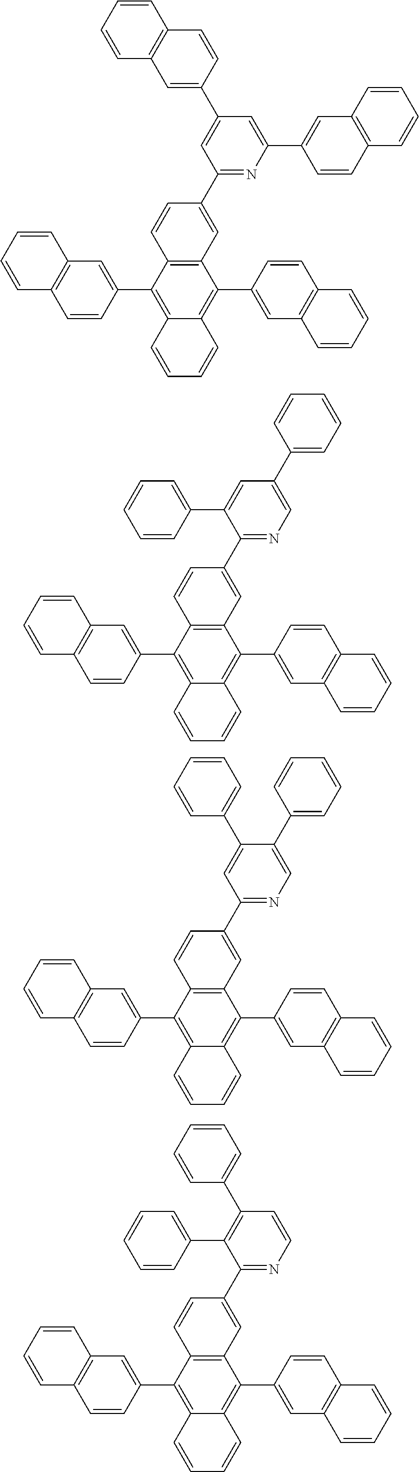

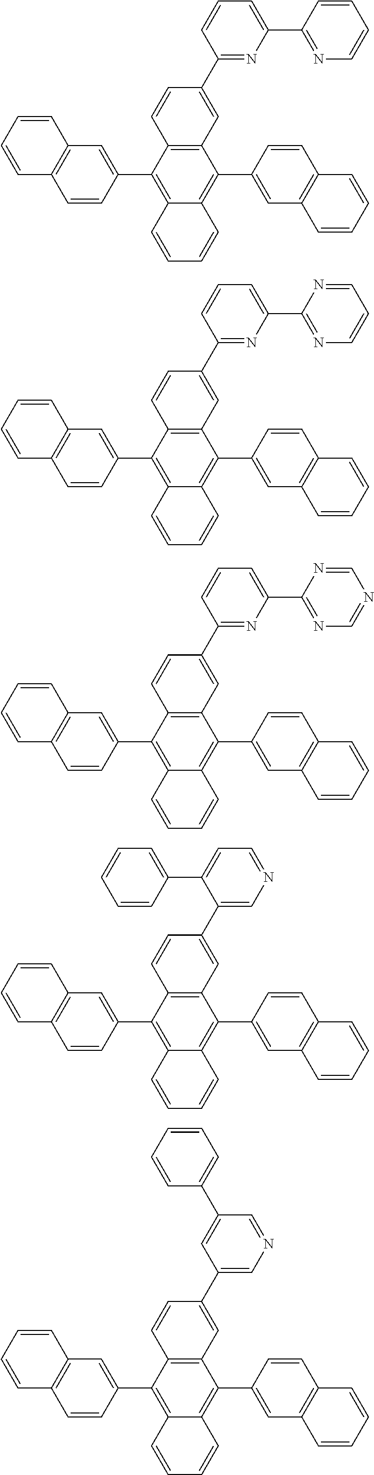

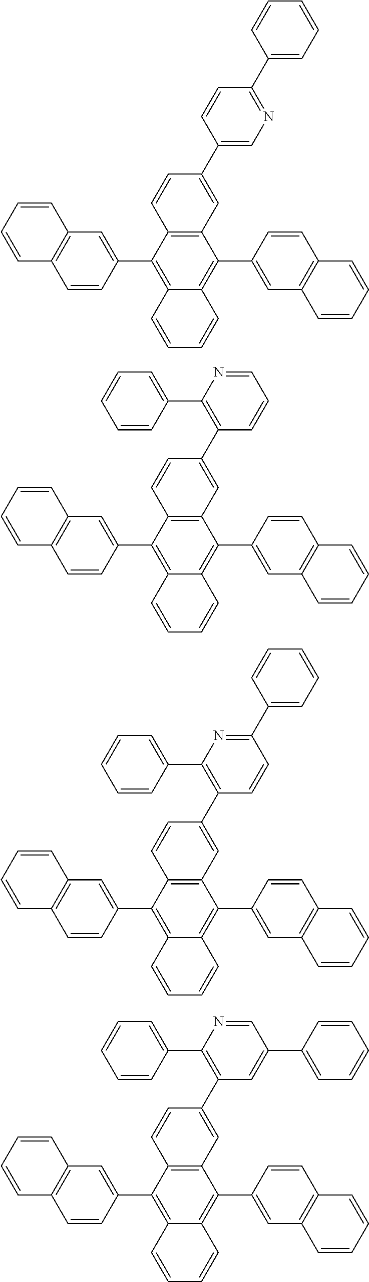

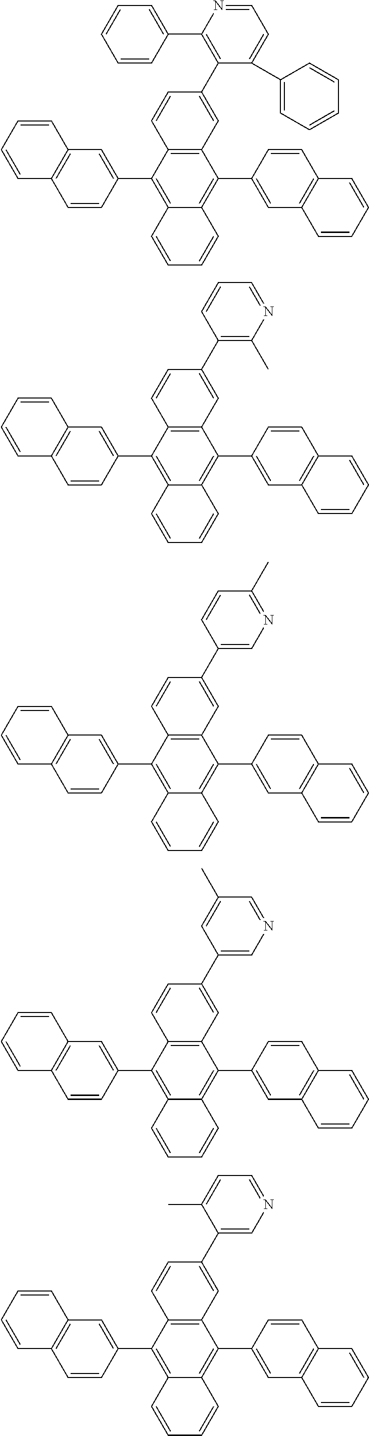

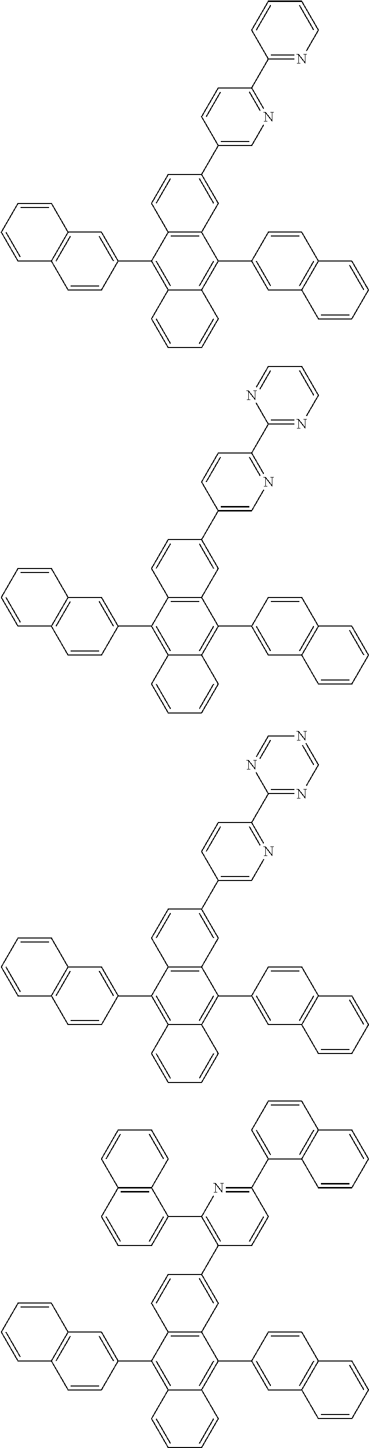 [SMILES: c1ccc(-c2ccc(-c3ccc4c(-c5ccc6ccccc6c5)c5ccccc5c(-c5ccc6ccccc6c5)c4c3)cn2)nc1 ; c1cnc(-c2ccc(-c3ccc4c(-c5ccc6ccccc6c5)c5ccccc5c(-c5ccc6ccccc6c5)c4c3)cn2)nc1 ; c1ccc2cc(-c3c4ccccc4c(-c4ccc5ccccc5c4)c4cc(-c5ccc(-c6ncncn6)nc5)ccc34)ccc2c1 ; c1ccc2cc(-c3c4ccccc4c(-c4ccc5ccccc5c4)c4cc(-c5ccc(-c6cccc7ccccc67)nc5-c5cccc6ccccc56)ccc34)ccc2c1]

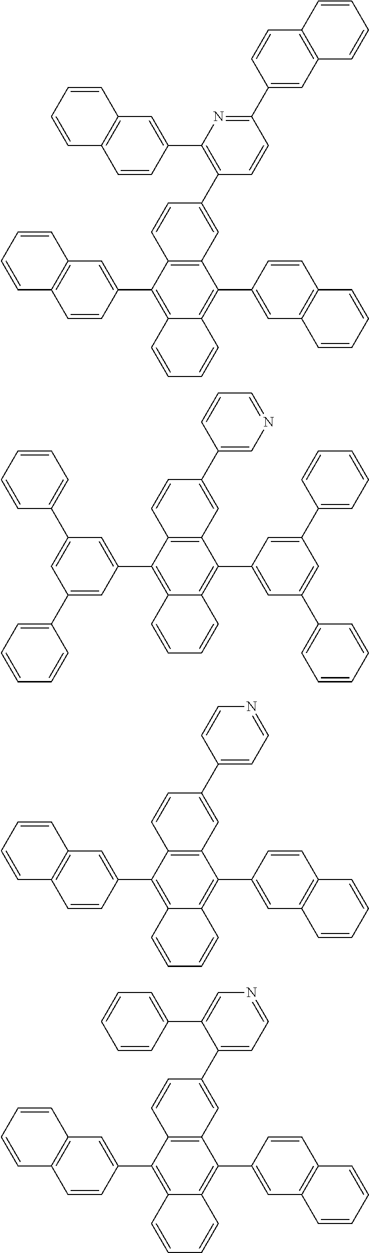

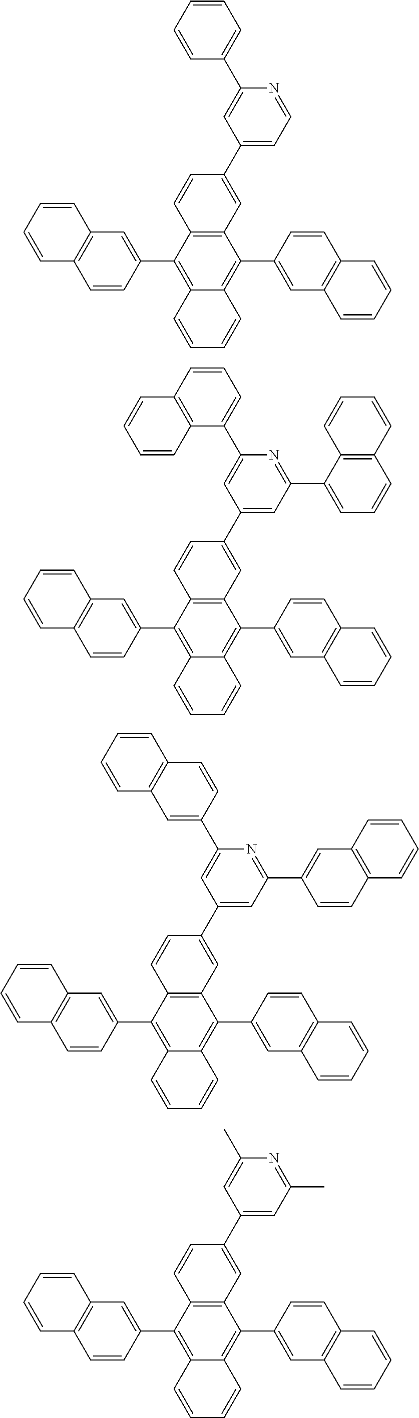

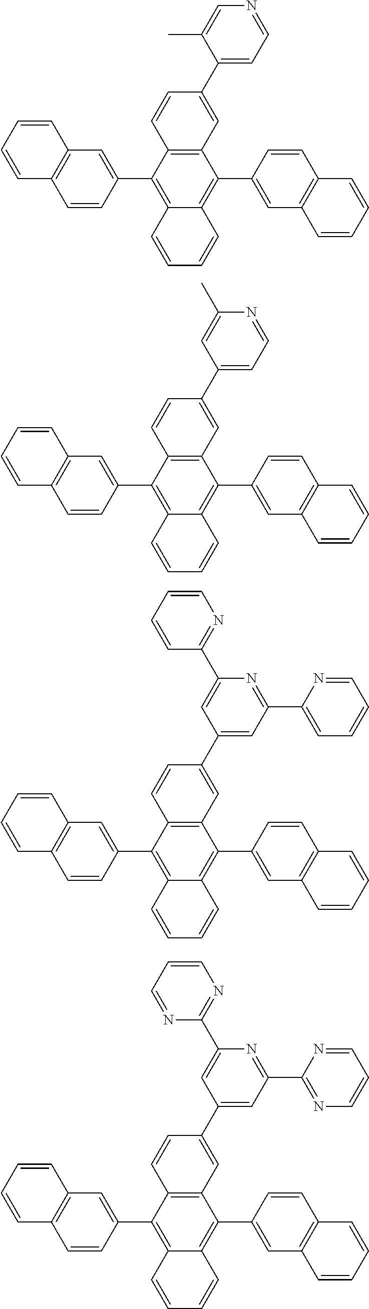

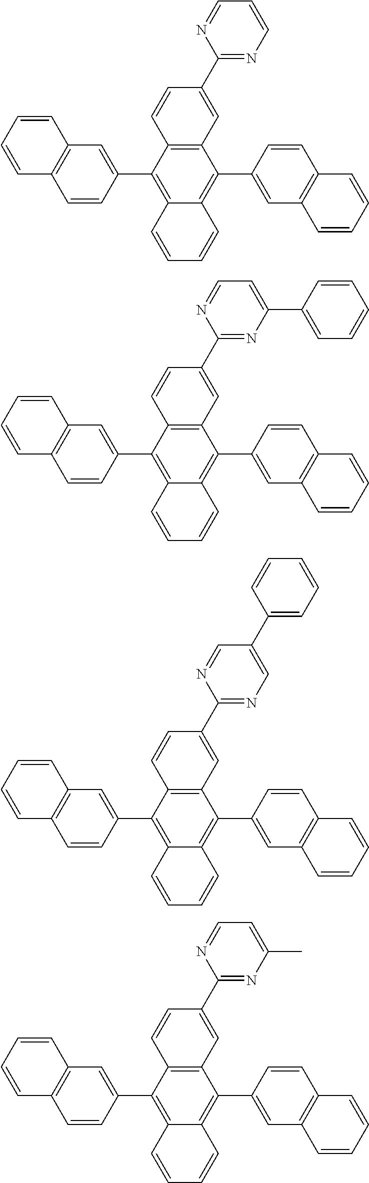

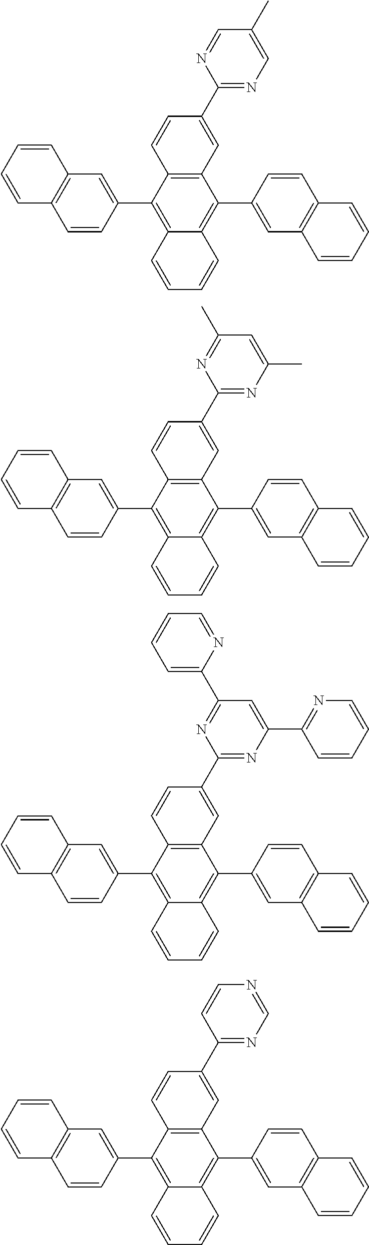

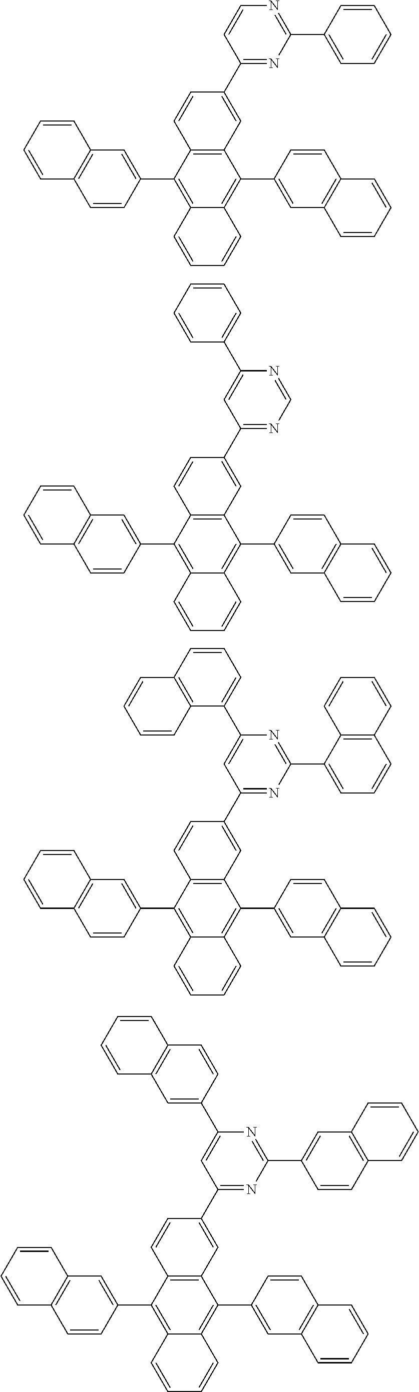

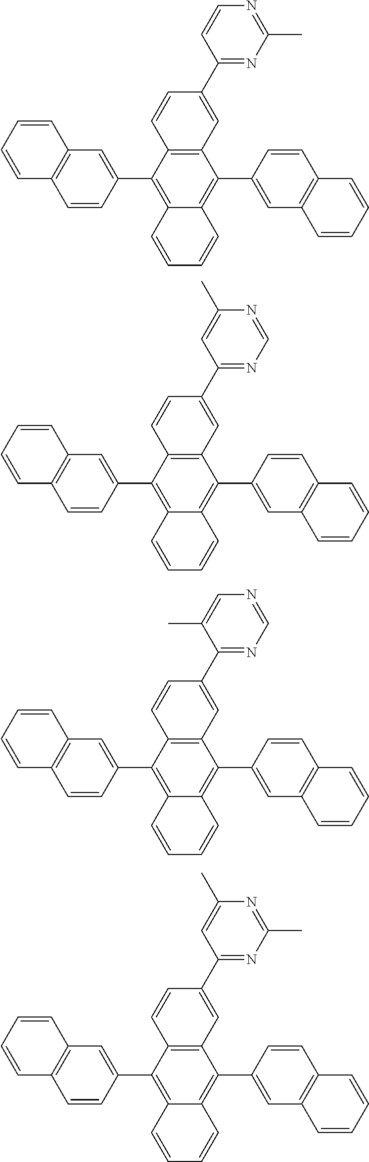

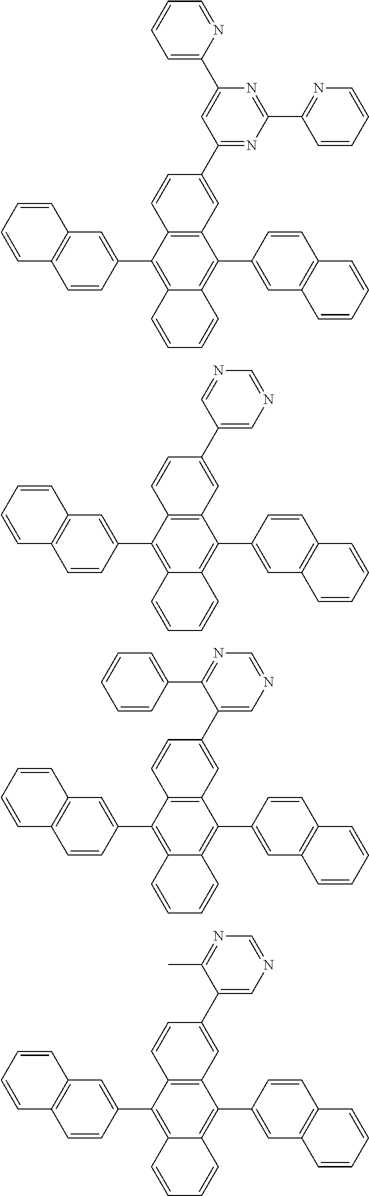

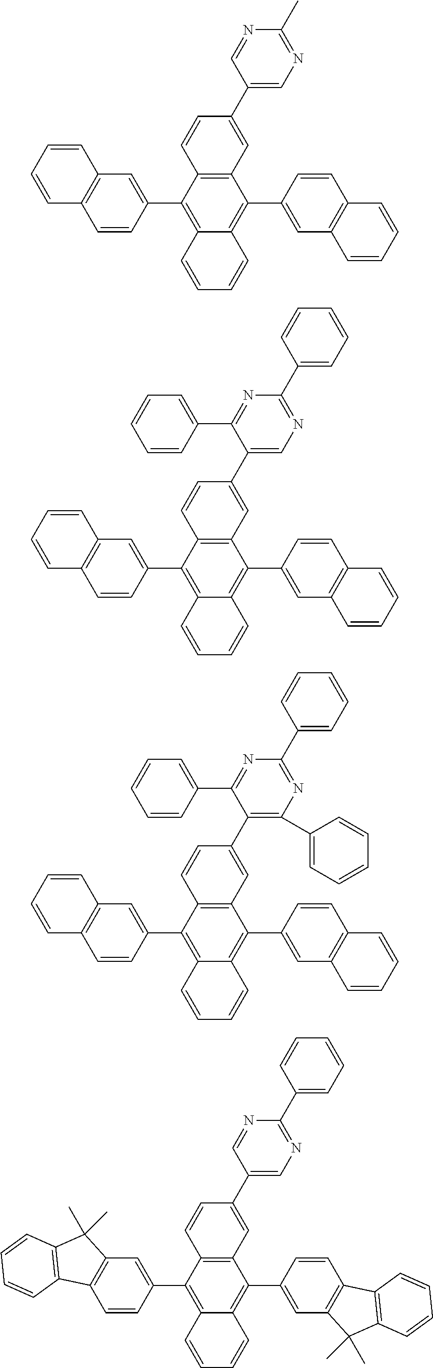 [SMILES: Cc1ncc(-c2ccc3c(-c4ccc5ccccc5c4)c4ccccc4c(-c4ccc5ccccc5c4)c3c2)cn1 ; c1ccc(-c2ncc(-c3ccc4c(-c5ccc6ccccc6c5)c5ccccc5c(-c5ccc6ccccc6c5)c4c3)c(-c3ccccc3)n2)cc1 ; c1ccc(-c2nc(-c3ccccc3)c(-c3ccc4c(-c5ccc6ccccc6c5)c5ccccc5c(-c5ccc6ccccc6c5)c4c3)c(-c3ccccc3)n2)cc1 ; CC1(C)c2ccccc2-c2ccc(-c3c4ccccc4c(-c4ccc5c(c4)C(C)(C)c4ccccc4-5)c4cc(-c5cnc(-c6ccccc6)nc5)ccc34)cc21]

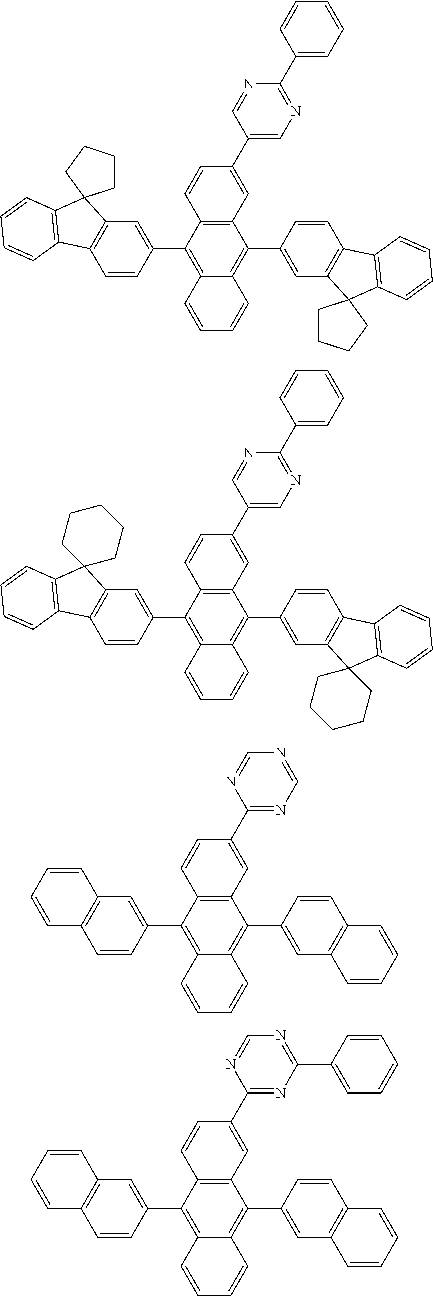

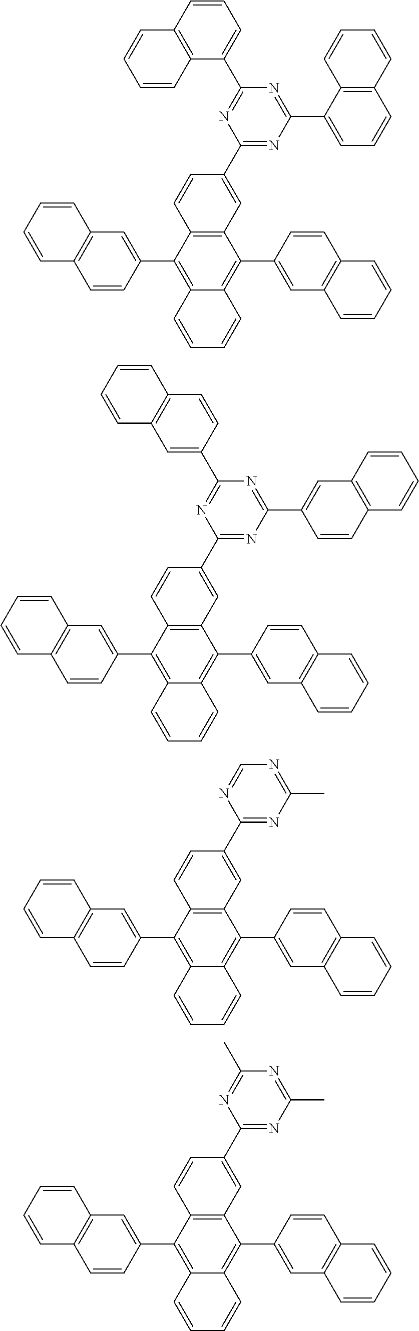

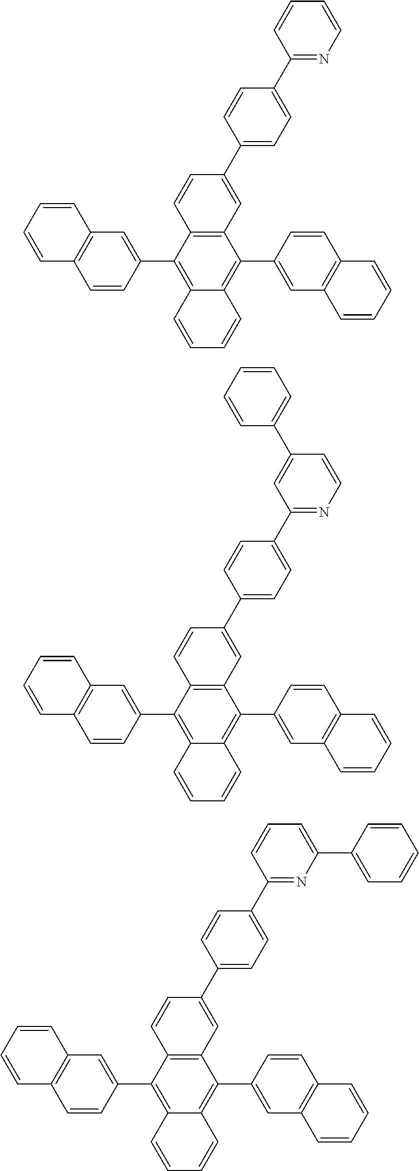

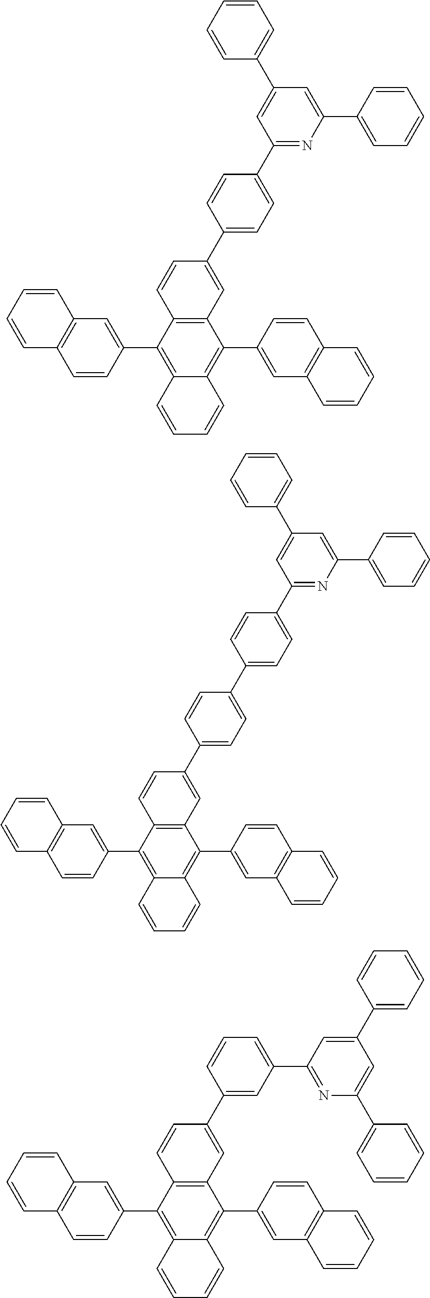

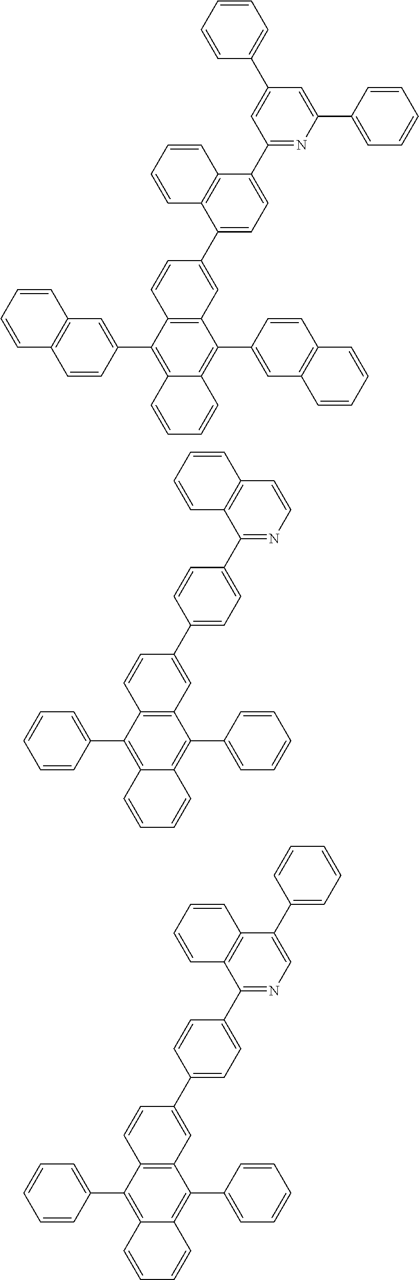

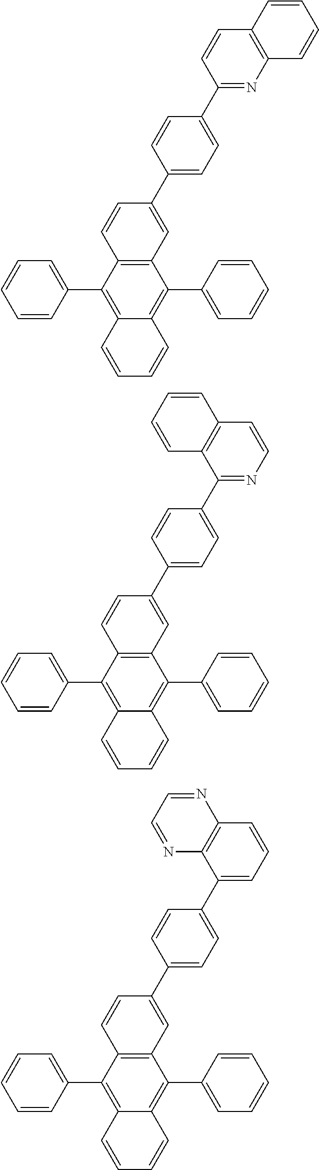

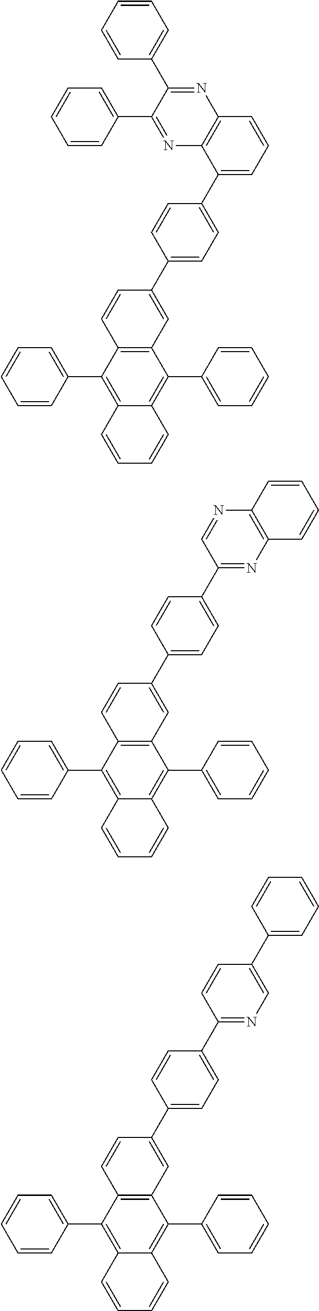 [SMILES: c1ccc(-c2nc3cccc(-c4ccc(-c5ccc6c(-c7ccccc7)c7ccccc7c(-c7ccccc7)c6c5)cc4)c3nc2-c2ccccc2)cc1 ; c1ccc(-c2c3ccccc3c(-c3ccccc3)c3cc(-c4ccc(-c5cnc6ccccc6n5)cc4)ccc23)cc1 ; c1ccc(-c2ccc(-c3ccc(-c4ccc5c(-c6ccccc6)c6ccccc6c(-c6ccccc6)c5c4)cc3)nc2)cc1]

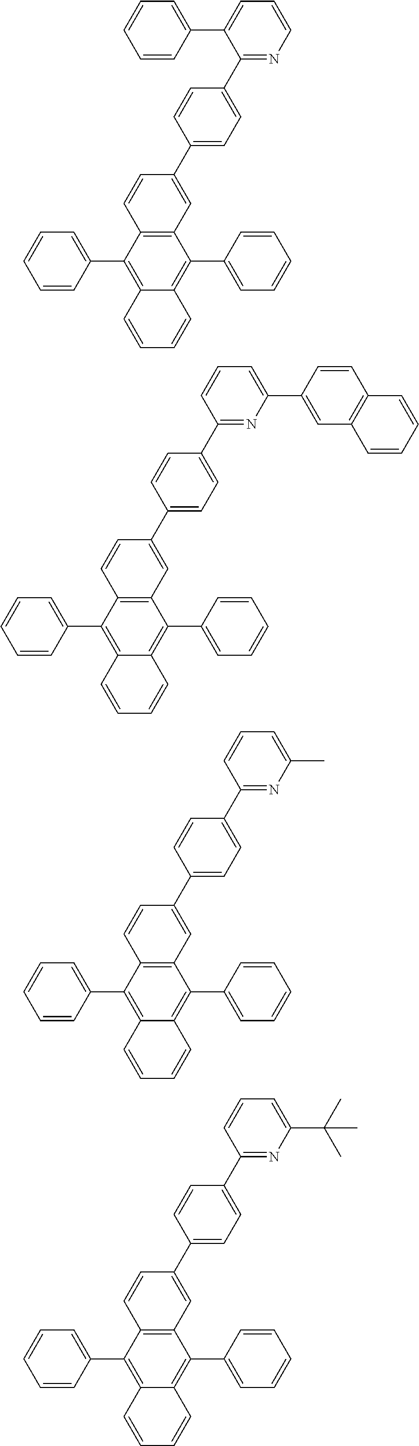 [SMILES: c1ccc(-c2cccnc2-c2ccc(-c3ccc4c(-c5ccccc5)c5ccccc5c(-c5ccccc5)c4c3)cc2)cc1 ; c1ccc(-c2c3ccccc3c(-c3ccccc3)c3cc(-c4ccc(-c5cccc(-c6ccc7ccccc7c6)n5)cc4)ccc23)cc1 ; Cc1cccc(-c2ccc(-c3ccc4c(-c5ccccc5)c5ccccc5c(-c5ccccc5)c4c3)cc2)n1 ; CC(C)(C)c1cccc(-c2ccc(-c3ccc4c(-c5ccccc5)c5ccccc5c(-c5ccccc5)c4c3)cc2)n1]

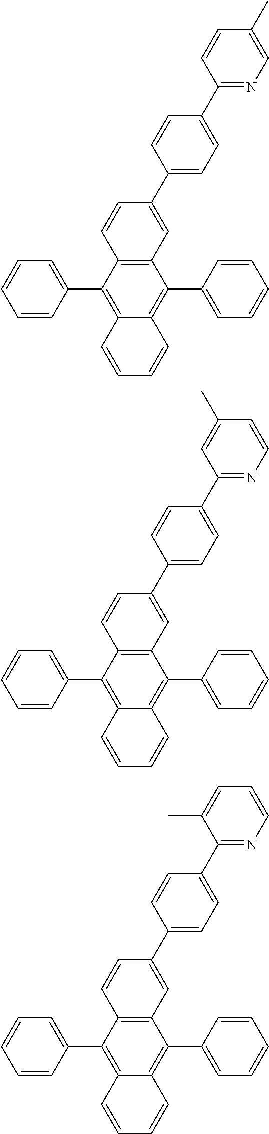 [SMILES: Cc1ccc(-c2ccc(-c3ccc4c(-c5ccccc5)c5ccccc5c(-c5ccccc5)c4c3)cc2)nc1 ; Cc1ccnc(-c2ccc(-c3ccc4c(-c5ccccc5)c5ccccc5c(-c5ccccc5)c4c3)cc2)c1 ; Cc1cccnc1-c1ccc(-c2ccc3c(-c4ccccc4)c4ccccc4c(-c4ccccc4)c3c2)cc1]

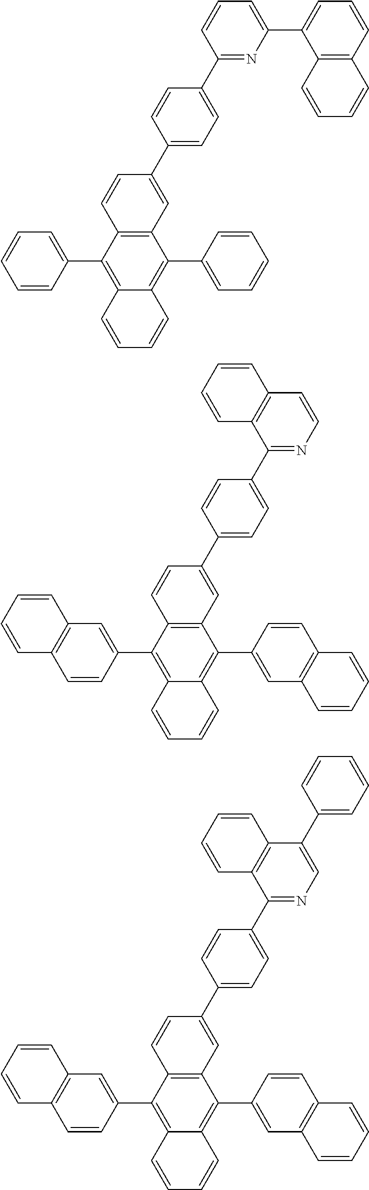

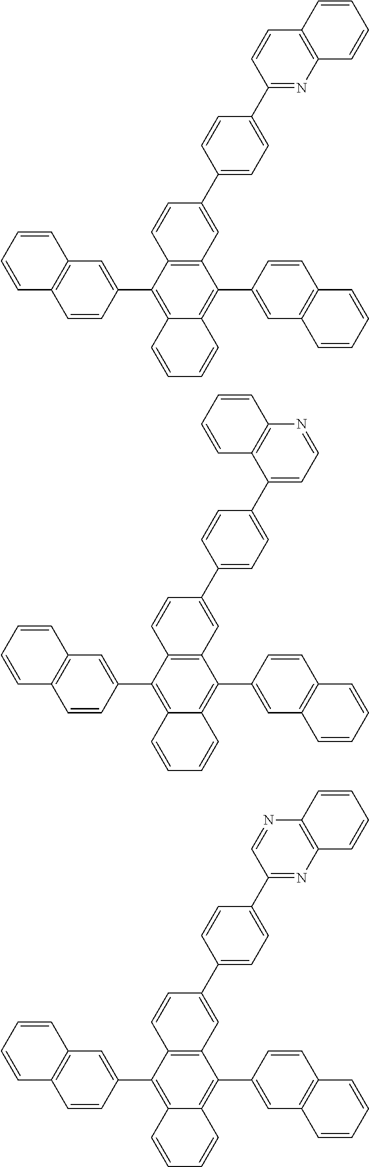

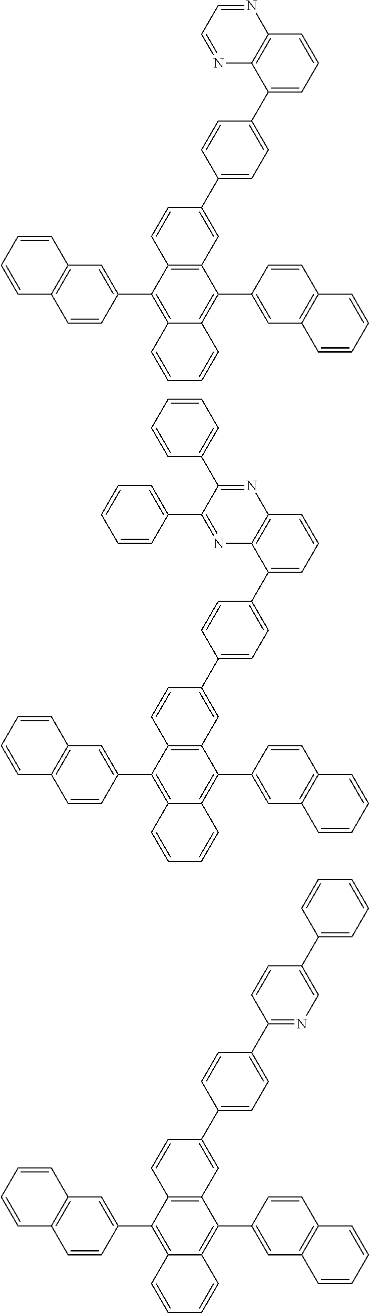

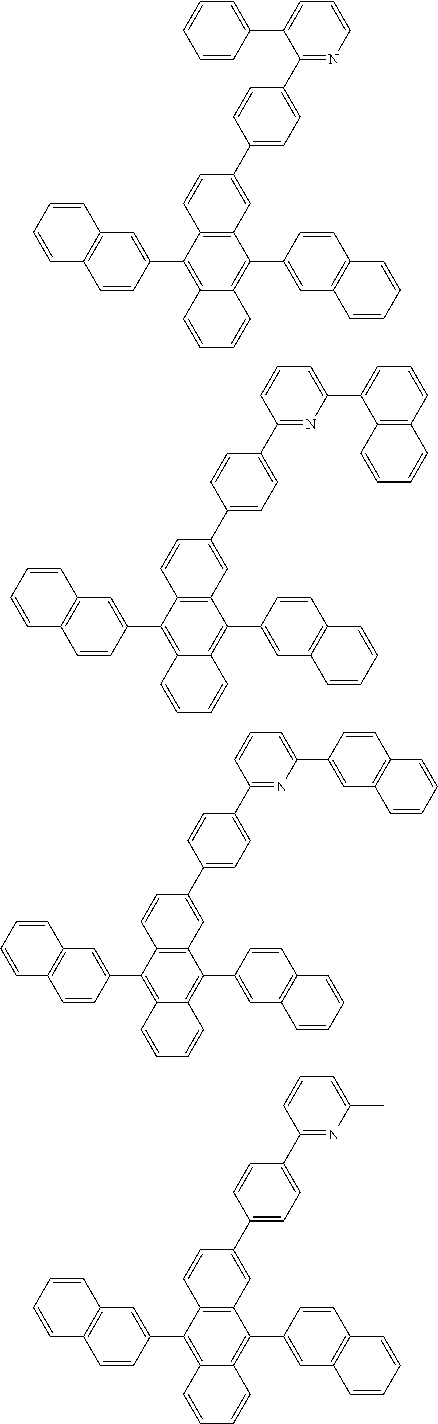

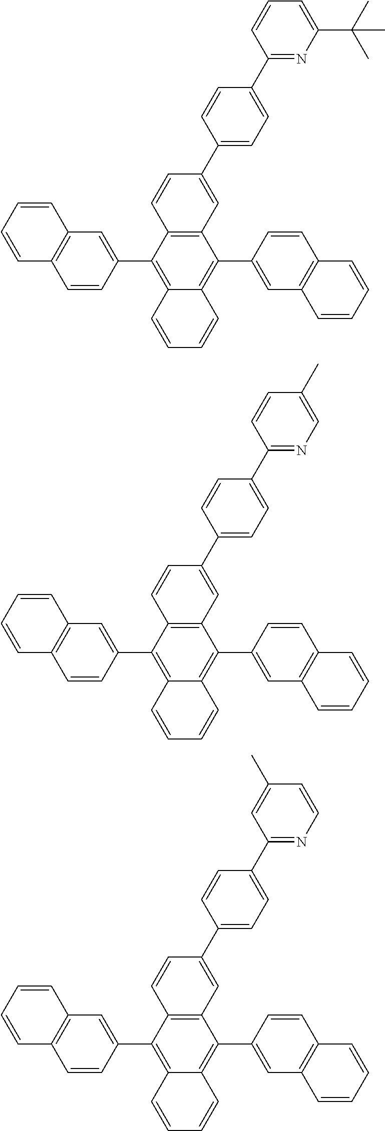

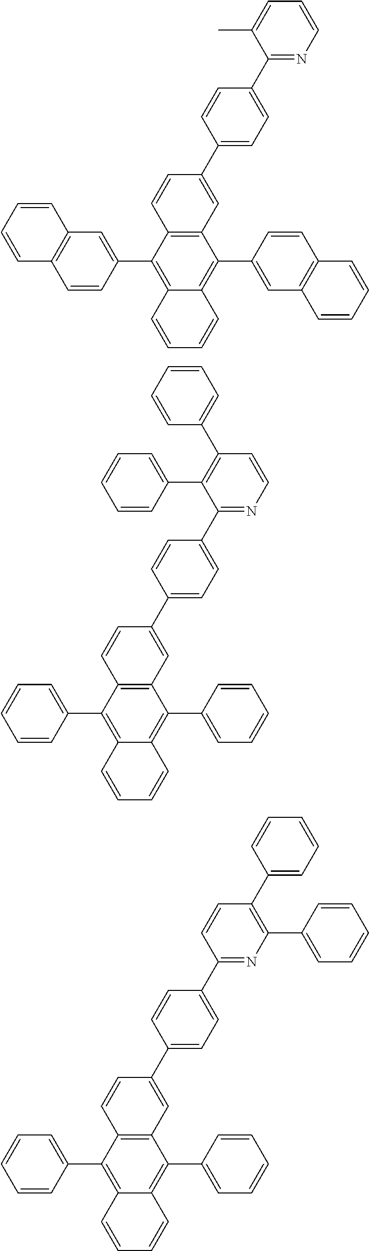

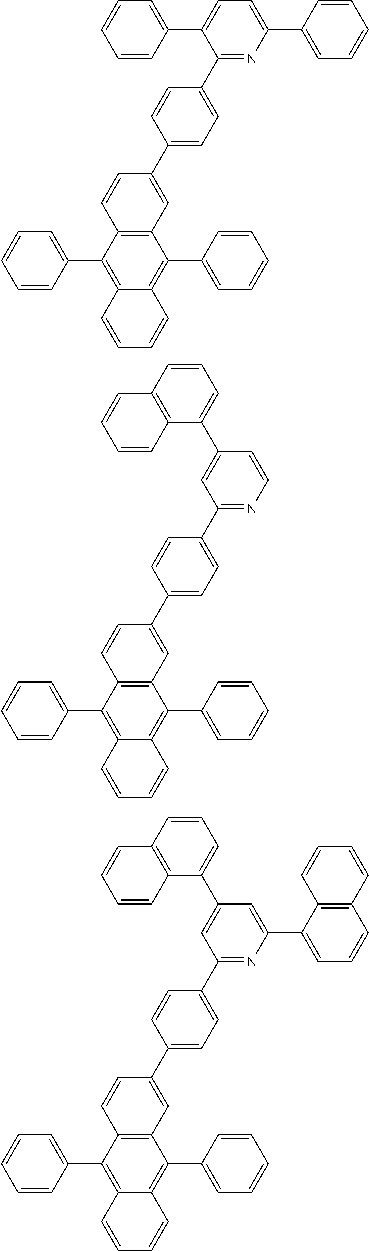

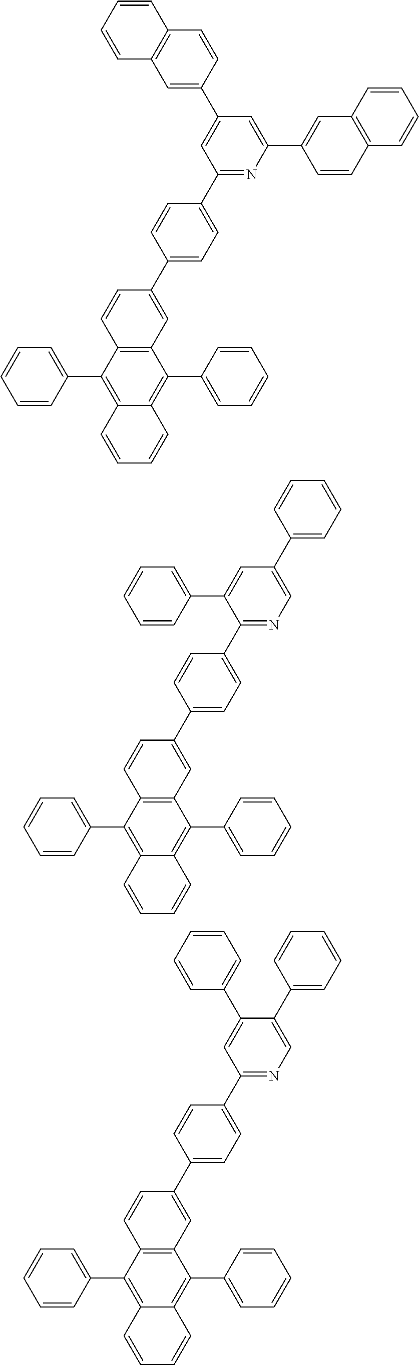

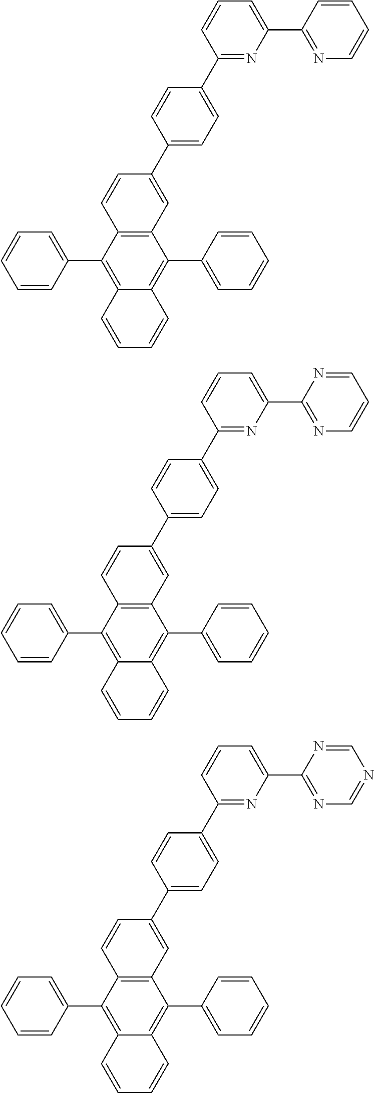 [SMILES: c1ccc(-c2c3ccccc3c(-c3ccccc3)c3cc(-c4ccc(-c5cccc(-c6ccccn6)n5)cc4)ccc23)cc1 ; c1ccc(-c2c3ccccc3c(-c3ccccc3)c3cc(-c4ccc(-c5cccc(-c6ncccn6)n5)cc4)ccc23)cc1 ; c1ccc(-c2c3ccccc3c(-c3ccccc3)c3cc(-c4ccc(-c5cccc(-c6ncncn6)n5)cc4)ccc23)cc1]

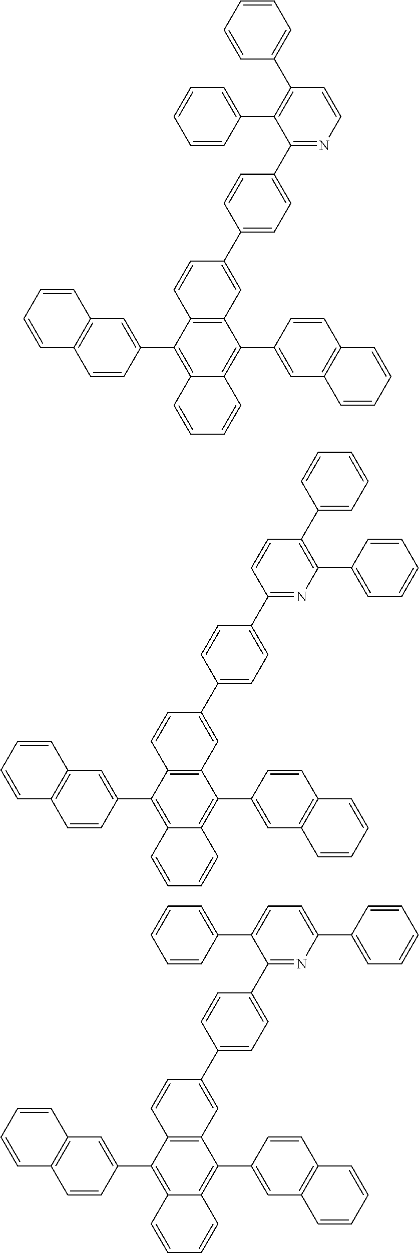

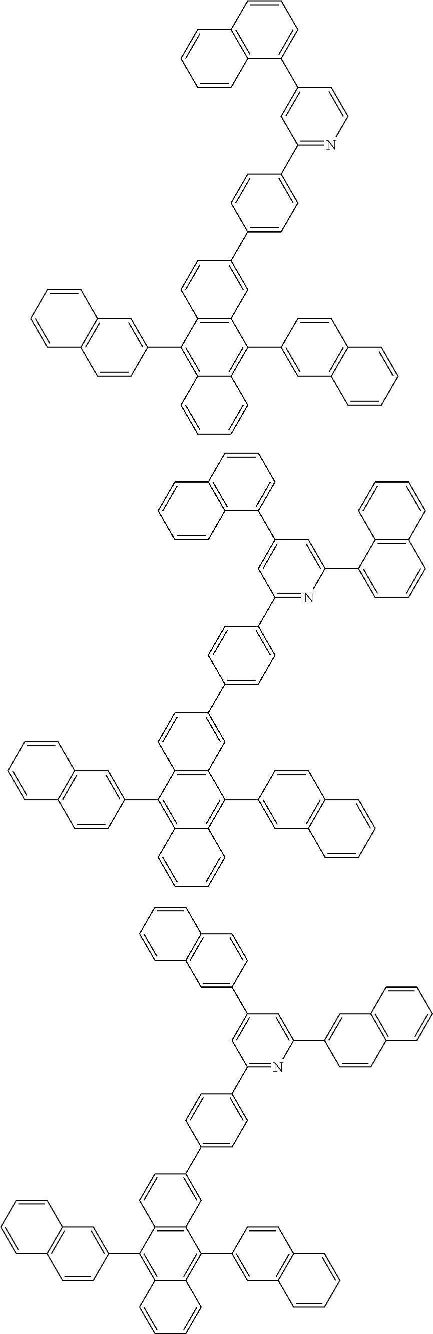

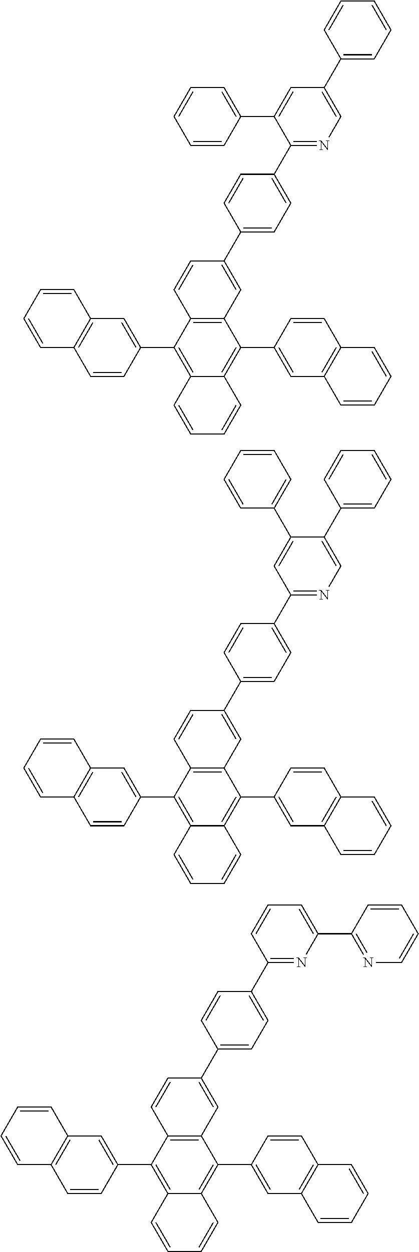

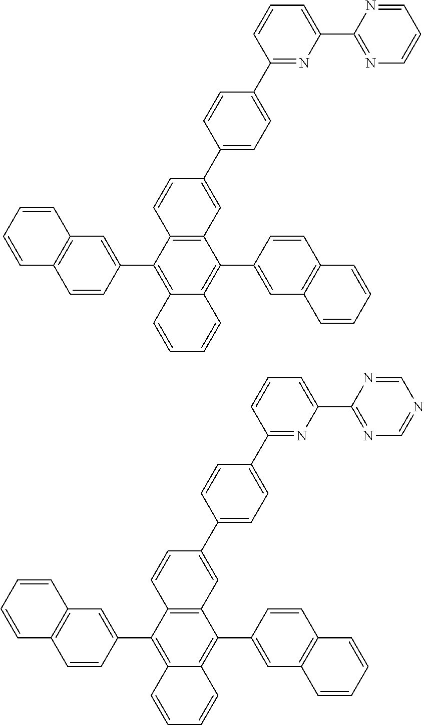 [SMILES: c1cnc(-c2cccc(-c3ccc(-c4ccc5c(-c6ccc7ccccc7c6)c6ccccc6c(-c6ccc7ccccc7c6)c5c4)cc3)n2)nc1 ; c1cc(-c2ccc(-c3ccc4c(-c5ccc6ccccc6c5)c5ccccc5c(-c5ccc6ccccc6c5)c4c3)cc2)nc(-c2ncncn2)c1]

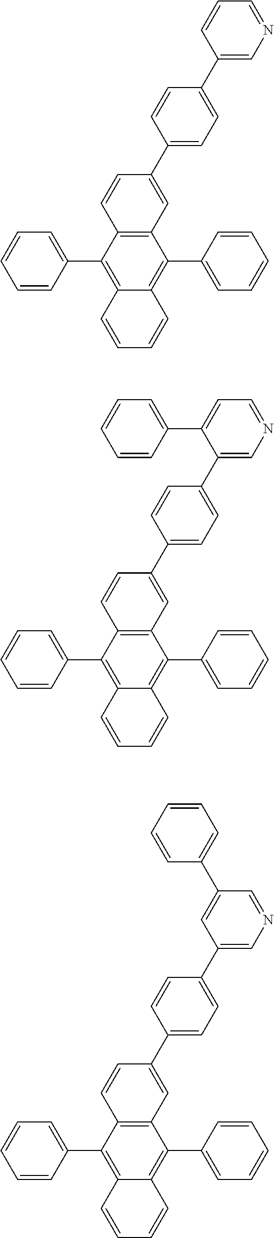 [SMILES: c1ccc(-c2c3ccccc3c(-c3ccccc3)c3cc(-c4ccc(-c5cccnc5)cc4)ccc23)cc1 ; c1ccc(-c2ccncc2-c2ccc(-c3ccc4c(-c5ccccc5)c5ccccc5c(-c5ccccc5)c4c3)cc2)cc1 ; c1ccc(-c2cncc(-c3ccc(-c4ccc5c(-c6ccccc6)c6ccccc6c(-c6ccccc6)c5c4)cc3)c2)cc1]

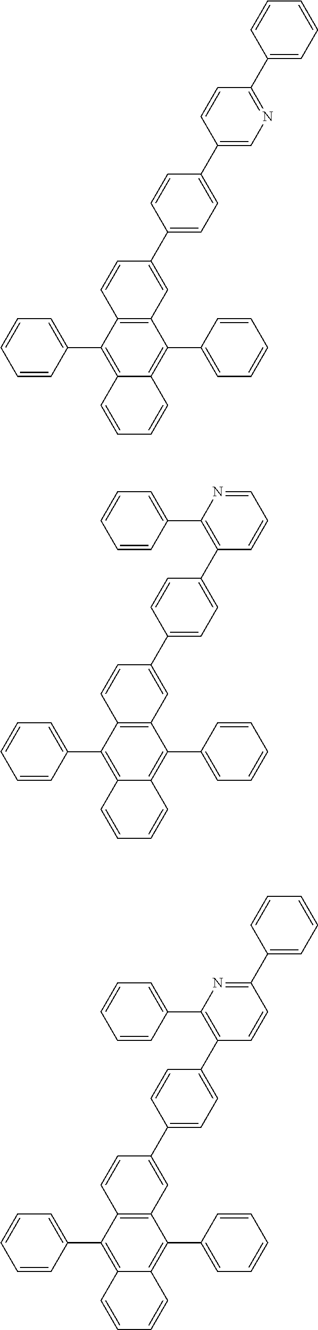 [SMILES: c1ccc(-c2ccc(-c3ccc(-c4ccc5c(-c6ccccc6)c6ccccc6c(-c6ccccc6)c5c4)cc3)cn2)cc1 ; c1ccc(-c2ncccc2-c2ccc(-c3ccc4c(-c5ccccc5)c5ccccc5c(-c5ccccc5)c4c3)cc2)cc1 ; c1ccc(-c2ccc(-c3ccc(-c4ccc5c(-c6ccccc6)c6ccccc6c(-c6ccccc6)c5c4)cc3)c(-c3ccccc3)n2)cc1]

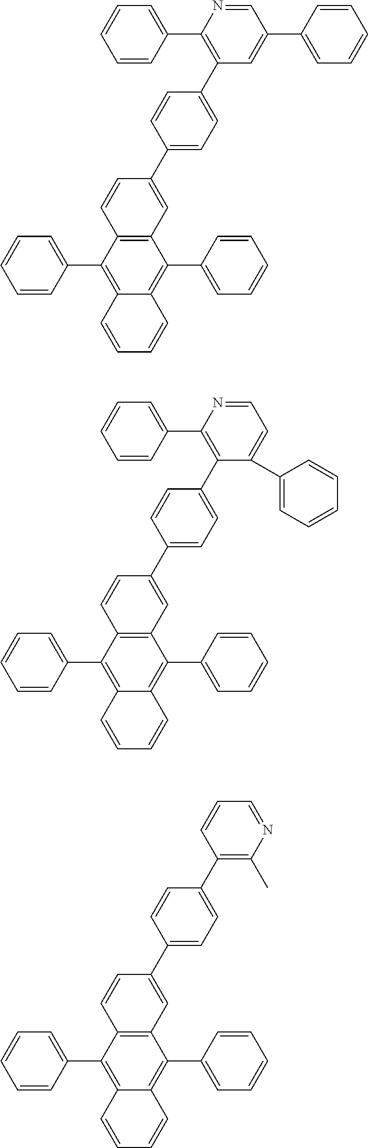 [SMILES: c1ccc(-c2cnc(-c3ccccc3)c(-c3ccc(-c4ccc5c(-c6ccccc6)c6ccccc6c(-c6ccccc6)c5c4)cc3)c2)cc1 ; c1ccc(-c2ccnc(-c3ccccc3)c2-c2ccc(-c3ccc4c(-c5ccccc5)c5ccccc5c(-c5ccccc5)c4c3)cc2)cc1 ; Cc1ncccc1-c1ccc(-c2ccc3c(-c4ccccc4)c4ccccc4c(-c4ccccc4)c3c2)cc1]

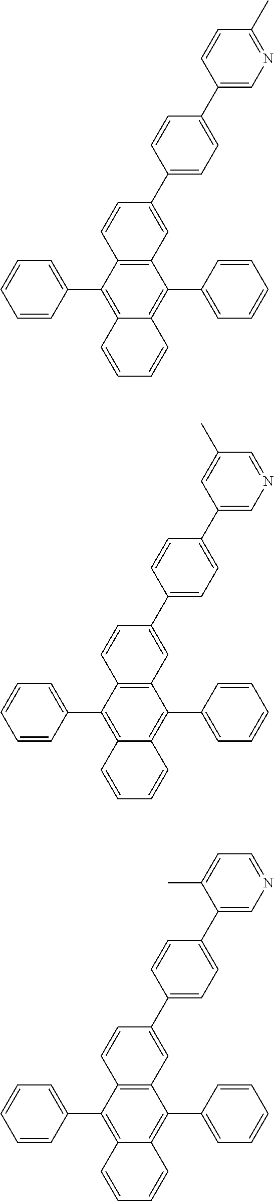 [SMILES: Cc1ccc(-c2ccc(-c3ccc4c(-c5ccccc5)c5ccccc5c(-c5ccccc5)c4c3)cc2)cn1 ; Cc1cncc(-c2ccc(-c3ccc4c(-c5ccccc5)c5ccccc5c(-c5ccccc5)c4c3)cc2)c1 ; Cc1ccncc1-c1ccc(-c2ccc3c(-c4ccccc4)c4ccccc4c(-c4ccccc4)c3c2)cc1]

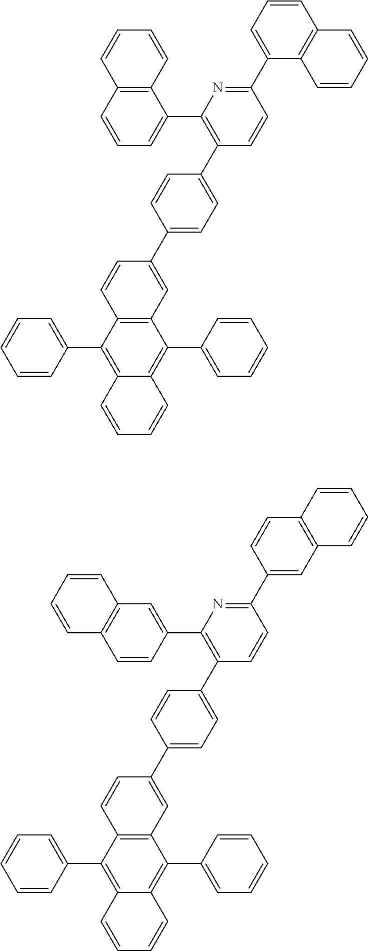 [SMILES: c1ccc(-c2c3ccccc3c(-c3ccccc3)c3cc(-c4ccc(-c5ccc(-c6cccc7ccccc67)nc5-c5cccc6ccccc56)cc4)ccc23)cc1 ; c1ccc(-c2c3ccccc3c(-c3ccccc3)c3cc(-c4ccc(-c5ccc(-c6ccc7ccccc7c6)nc5-c5ccc6ccccc6c5)cc4)ccc23)cc1]

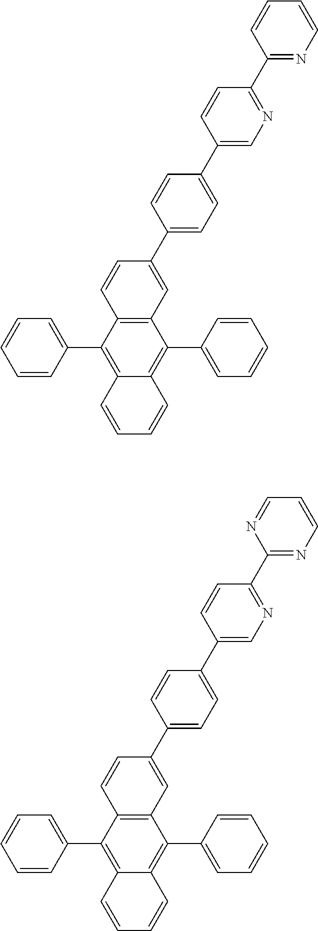 [SMILES: c1ccc(-c2c3ccccc3c(-c3ccccc3)c3cc(-c4ccc(-c5ccc(-c6ccccn6)nc5)cc4)ccc23)cc1 ; c1ccc(-c2c3ccccc3c(-c3ccccc3)c3cc(-c4ccc(-c5ccc(-c6ncccn6)nc5)cc4)ccc23)cc1]

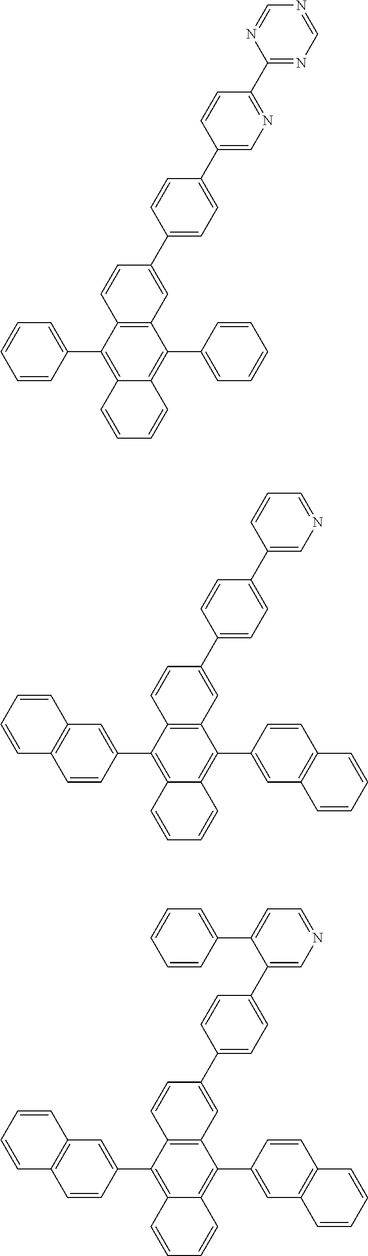 [SMILES: c1ccc(-c2c3ccccc3c(-c3ccccc3)c3cc(-c4ccc(-c5ccc(-c6ncncn6)nc5)cc4)ccc23)cc1 ; c1cncc(-c2ccc(-c3ccc4c(-c5ccc6ccccc6c5)c5ccccc5c(-c5ccc6ccccc6c5)c4c3)cc2)c1 ; c1ccc(-c2ccncc2-c2ccc(-c3ccc4c(-c5ccc6ccccc6c5)c5ccccc5c(-c5ccc6ccccc6c5)c4c3)cc2)cc1]

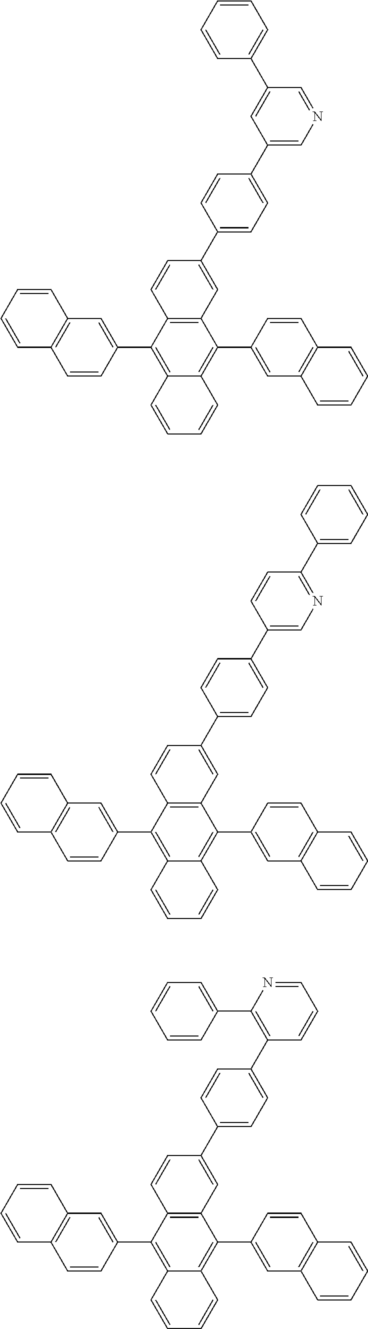 [SMILES: c1ccc(-c2cncc(-c3ccc(-c4ccc5c(-c6ccc7ccccc7c6)c6ccccc6c(-c6ccc7ccccc7c6)c5c4)cc3)c2)cc1 ; c1ccc(-c2ccc(-c3ccc(-c4ccc5c(-c6ccc7ccccc7c6)c6ccccc6c(-c6ccc7ccccc7c6)c5c4)cc3)cn2)cc1 ; c1ccc(-c2ncccc2-c2ccc(-c3ccc4c(-c5ccc6ccccc6c5)c5ccccc5c(-c5ccc6ccccc6c5)c4c3)cc2)cc1]

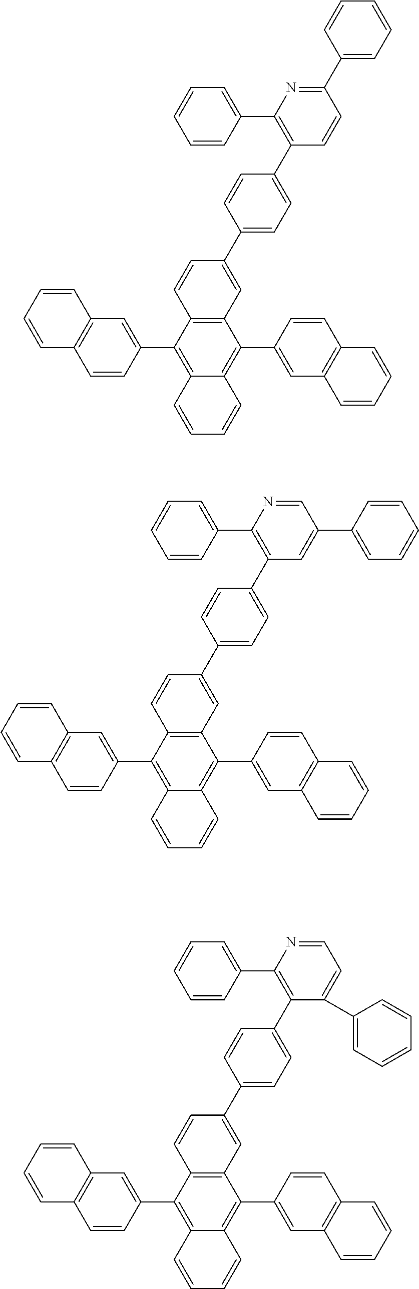 [SMILES: c1ccc(-c2ccc(-c3ccc(-c4ccc5c(-c6ccc7ccccc7c6)c6ccccc6c(-c6ccc7ccccc7c6)c5c4)cc3)c(-c3ccccc3)n2)cc1 ; c1ccc(-c2cnc(-c3ccccc3)c(-c3ccc(-c4ccc5c(-c6ccc7ccccc7c6)c6ccccc6c(-c6ccc7ccccc7c6)c5c4)cc3)c2)cc1 ; c1ccc(-c2ccnc(-c3ccccc3)c2-c2ccc(-c3ccc4c(-c5ccc6ccccc6c5)c5ccccc5c(-c5ccc6ccccc6c5)c4c3)cc2)cc1]

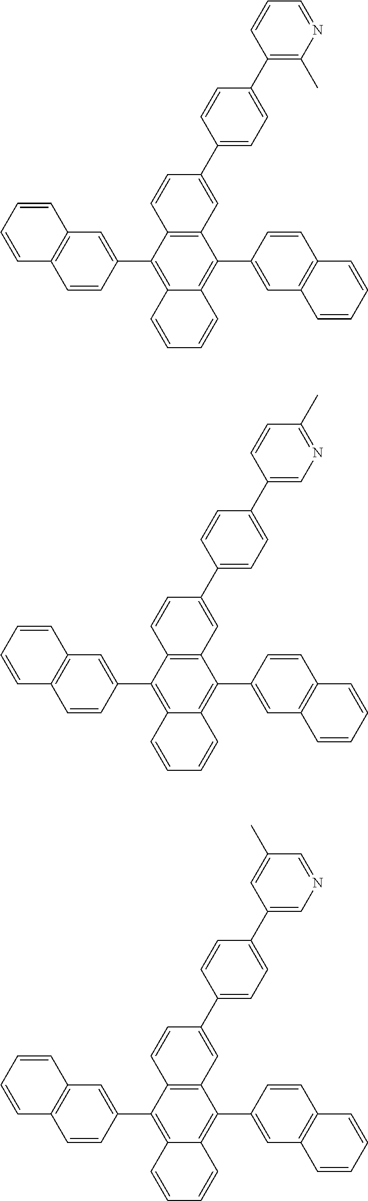 [SMILES: Cc1ncccc1-c1ccc(-c2ccc3c(-c4ccc5ccccc5c4)c4ccccc4c(-c4ccc5ccccc5c4)c3c2)cc1 ; Cc1ccc(-c2ccc(-c3ccc4c(-c5ccc6ccccc6c5)c5ccccc5c(-c5ccc6ccccc6c5)c4c3)cc2)cn1 ; Cc1cncc(-c2ccc(-c3ccc4c(-c5ccc6ccccc6c5)c5ccccc5c(-c5ccc6ccccc6c5)c4c3)cc2)c1]

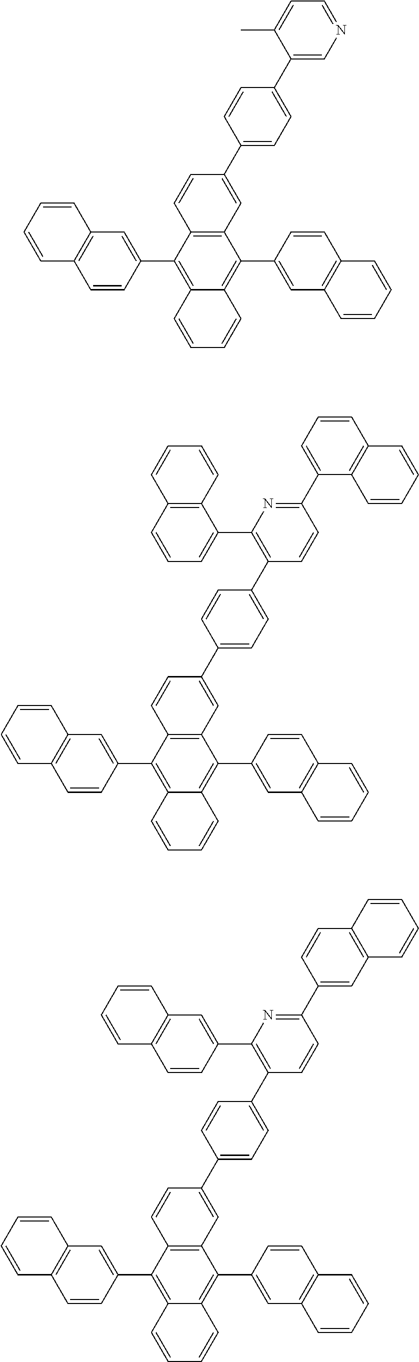 [SMILES: Cc1ccncc1-c1ccc(-c2ccc3c(-c4ccc5ccccc5c4)c4ccccc4c(-c4ccc5ccccc5c4)c3c2)cc1 ; c1ccc2cc(-c3c4ccccc4c(-c4ccc5ccccc5c4)c4cc(-c5ccc(-c6ccc(-c7cccc8ccccc78)nc6-c6cccc7ccccc67)cc5)ccc34)ccc2c1 ; c1ccc2cc(-c3ccc(-c4ccc(-c5ccc6c(-c7ccc8ccccc8c7)c7ccccc7c(-c7ccc8ccccc8c7)c6c5)cc4)c(-c4ccc5ccccc5c4)n3)ccc2c1]

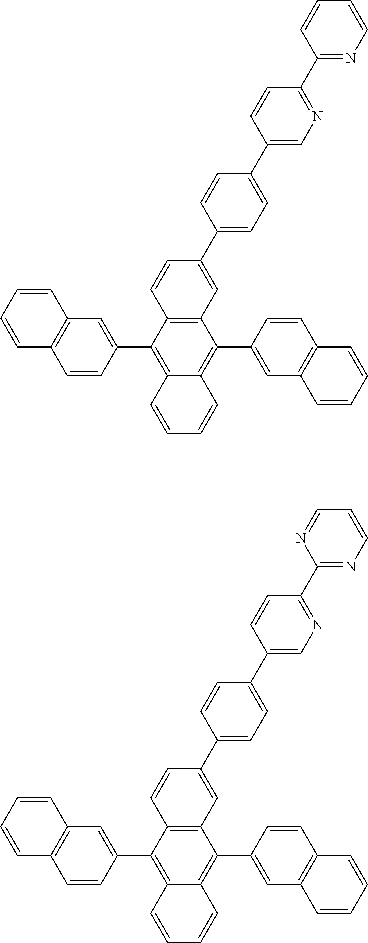 [SMILES: c1ccc(-c2ccc(-c3ccc(-c4ccc5c(-c6ccc7ccccc7c6)c6ccccc6c(-c6ccc7ccccc7c6)c5c4)cc3)cn2)nc1 ; c1cnc(-c2ccc(-c3ccc(-c4ccc5c(-c6ccc7ccccc7c6)c6ccccc6c(-c6ccc7ccccc7c6)c5c4)cc3)cn2)nc1]

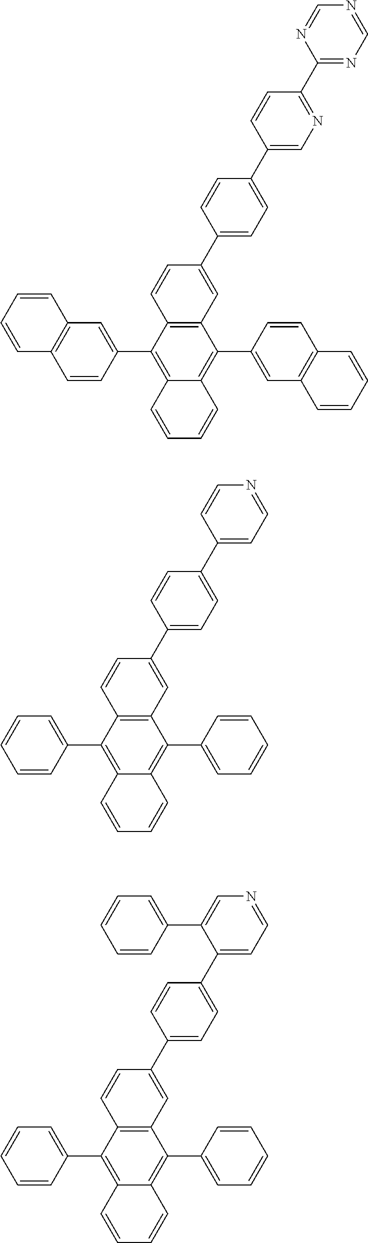 [SMILES: c1ccc2cc(-c3c4ccccc4c(-c4ccc5ccccc5c4)c4cc(-c5ccc(-c6ccc(-c7ncncn7)nc6)cc5)ccc34)ccc2c1 ; c1ccc(-c2c3ccccc3c(-c3ccccc3)c3cc(-c4ccc(-c5ccncc5)cc4)ccc23)cc1 ; c1ccc(-c2cnccc2-c2ccc(-c3ccc4c(-c5ccccc5)c5ccccc5c(-c5ccccc5)c4c3)cc2)cc1]

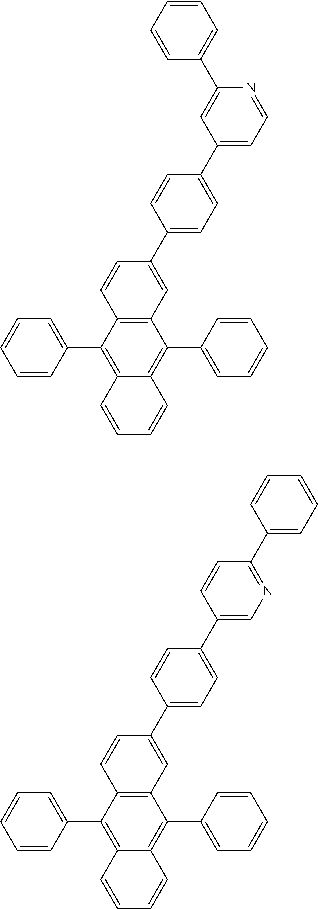 [SMILES: c1ccc(-c2cc(-c3ccc(-c4ccc5c(-c6ccccc6)c6ccccc6c(-c6ccccc6)c5c4)cc3)ccn2)cc1 ; c1ccc(-c2ccc(-c3ccc(-c4ccc5c(-c6ccccc6)c6ccccc6c(-c6ccccc6)c5c4)cc3)cn2)cc1]

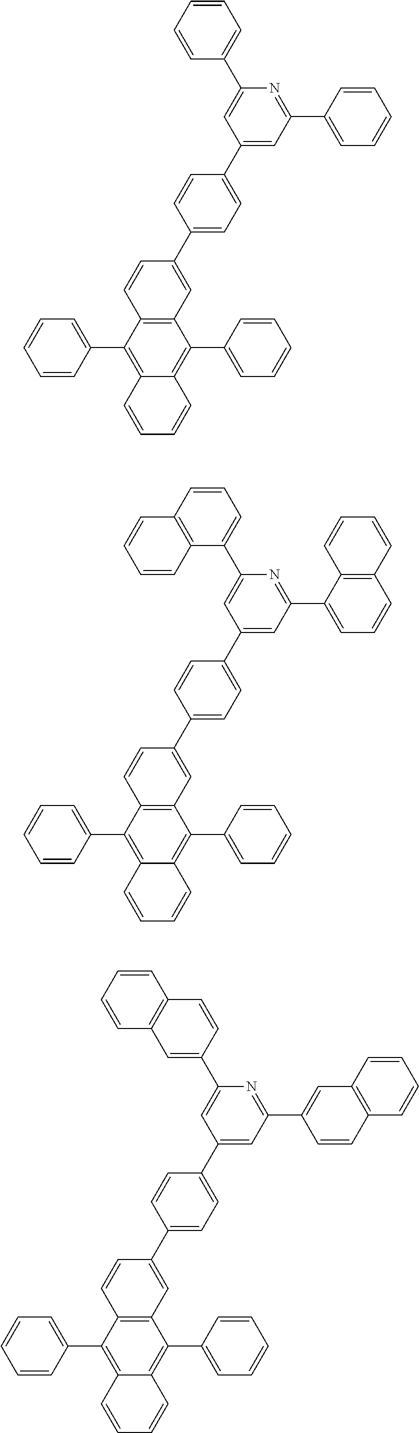 [SMILES: c1ccc(-c2cc(-c3ccc(-c4ccc5c(-c6ccccc6)c6ccccc6c(-c6ccccc6)c5c4)cc3)cc(-c3ccccc3)n2)cc1 ; c1ccc(-c2c3ccccc3c(-c3ccccc3)c3cc(-c4ccc(-c5cc(-c6cccc7ccccc67)nc(-c6cccc7ccccc67)c5)cc4)ccc23)cc1 ; c1ccc(-c2c3ccccc3c(-c3ccccc3)c3cc(-c4ccc(-c5cc(-c6ccc7ccccc7c6)nc(-c6ccc7ccccc7c6)c5)cc4)ccc23)cc1]

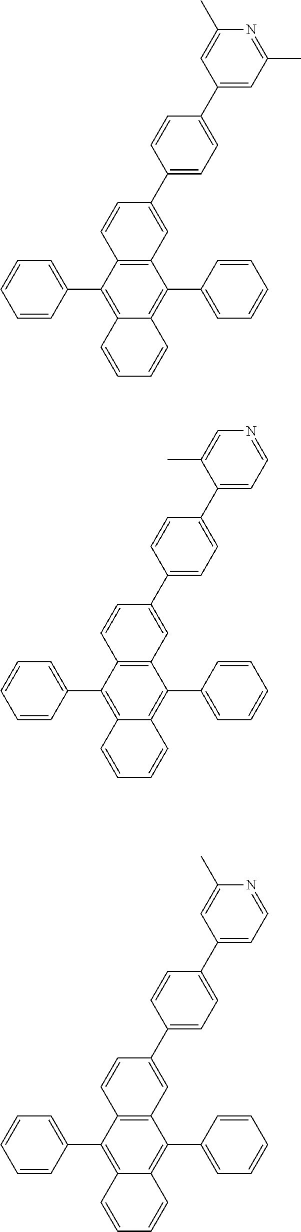 [SMILES: Cc1cc(-c2ccc(-c3ccc4c(-c5ccccc5)c5ccccc5c(-c5ccccc5)c4c3)cc2)cc(C)n1 ; Cc1cnccc1-c1ccc(-c2ccc3c(-c4ccccc4)c4ccccc4c(-c4ccccc4)c3c2)cc1 ; Cc1cc(-c2ccc(-c3ccc4c(-c5ccccc5)c5ccccc5c(-c5ccccc5)c4c3)cc2)ccn1]

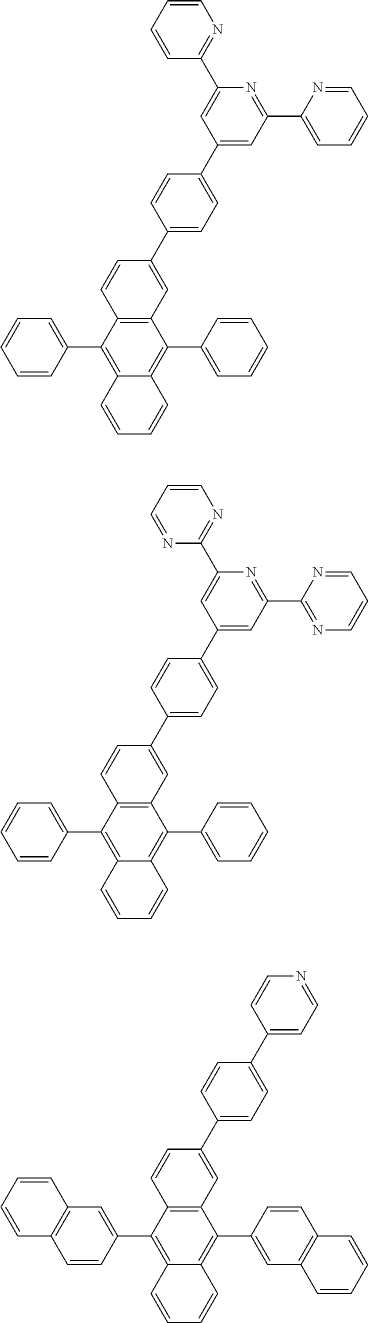 [SMILES: c1ccc(-c2c3ccccc3c(-c3ccccc3)c3cc(-c4ccc(-c5cc(-c6ccccn6)nc(-c6ccccn6)c5)cc4)ccc23)cc1 ; c1ccc(-c2c3ccccc3c(-c3ccccc3)c3cc(-c4ccc(-c5cc(-c6ncccn6)nc(-c6ncccn6)c5)cc4)ccc23)cc1 ; c1ccc2cc(-c3c4ccccc4c(-c4ccc5ccccc5c4)c4cc(-c5ccc(-c6ccncc6)cc5)ccc34)ccc2c1]

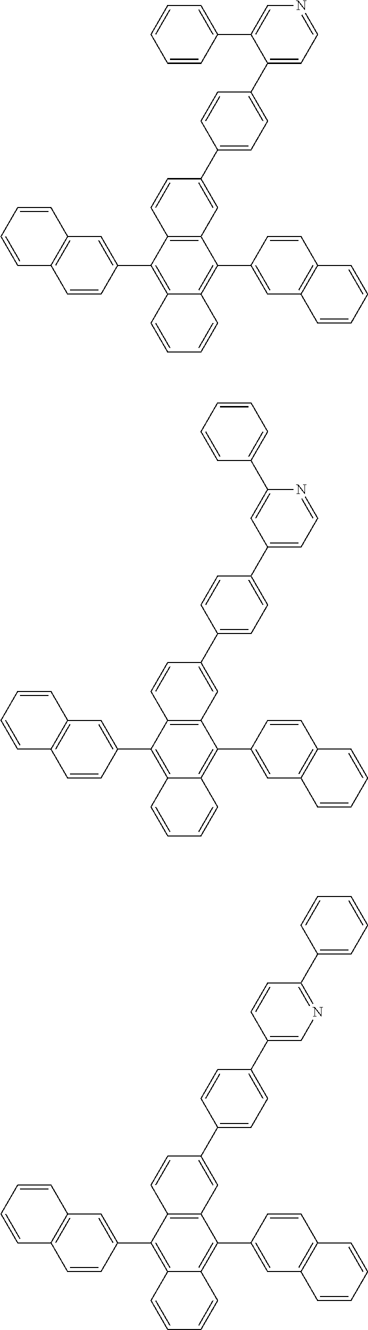 [SMILES: c1ccc(-c2cnccc2-c2ccc(-c3ccc4c(-c5ccc6ccccc6c5)c5ccccc5c(-c5ccc6ccccc6c5)c4c3)cc2)cc1 ; c1ccc(-c2cc(-c3ccc(-c4ccc5c(-c6ccc7ccccc7c6)c6ccccc6c(-c6ccc7ccccc7c6)c5c4)cc3)ccn2)cc1 ; c1ccc(-c2ccc(-c3ccc(-c4ccc5c(-c6ccc7ccccc7c6)c6ccccc6c(-c6ccc7ccccc7c6)c5c4)cc3)cn2)cc1]

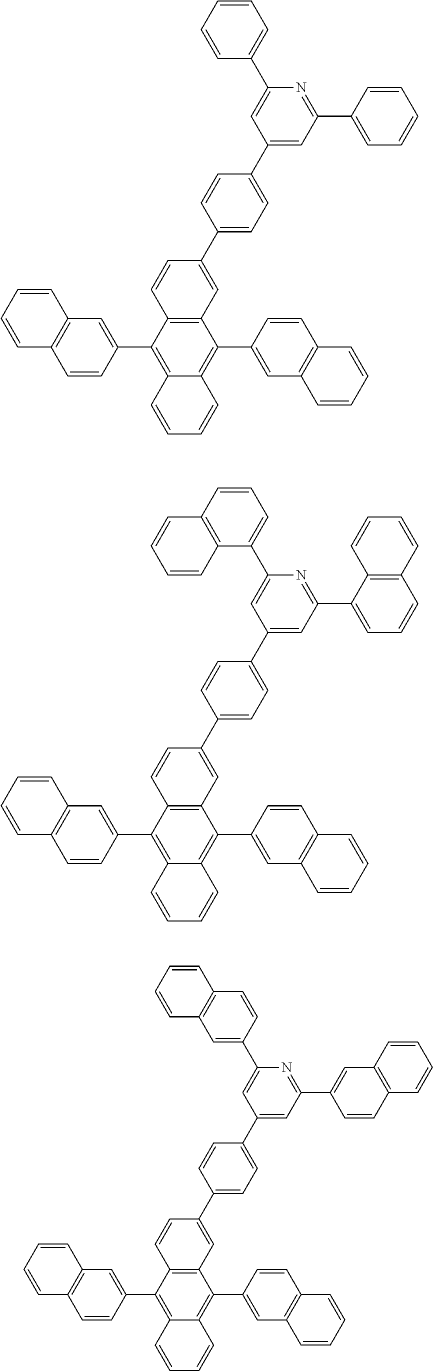

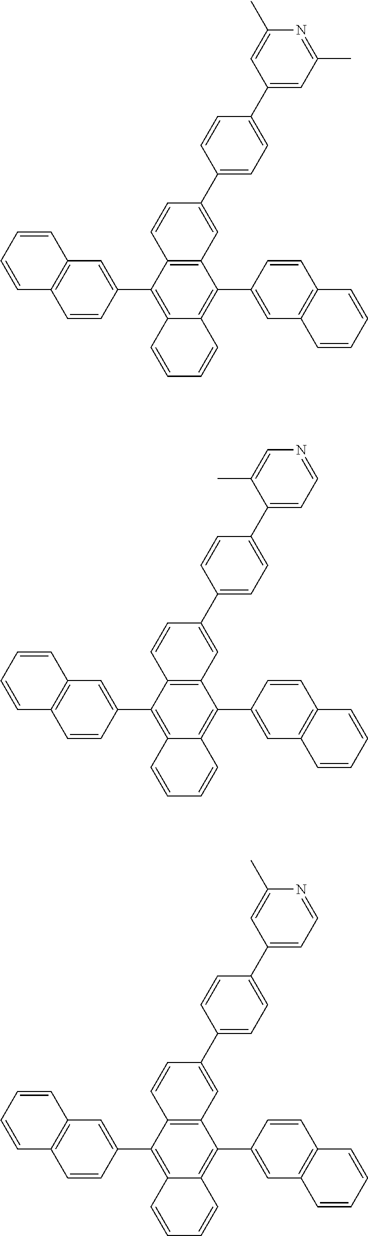 [SMILES: Cc1cc(-c2ccc(-c3ccc4c(-c5ccc6ccccc6c5)c5ccccc5c(-c5ccc6ccccc6c5)c4c3)cc2)cc(C)n1 ; Cc1cnccc1-c1ccc(-c2ccc3c(-c4ccc5ccccc5c4)c4ccccc4c(-c4ccc5ccccc5c4)c3c2)cc1 ; Cc1cc(-c2ccc(-c3ccc4c(-c5ccc6ccccc6c5)c5ccccc5c(-c5ccc6ccccc6c5)c4c3)cc2)ccn1]

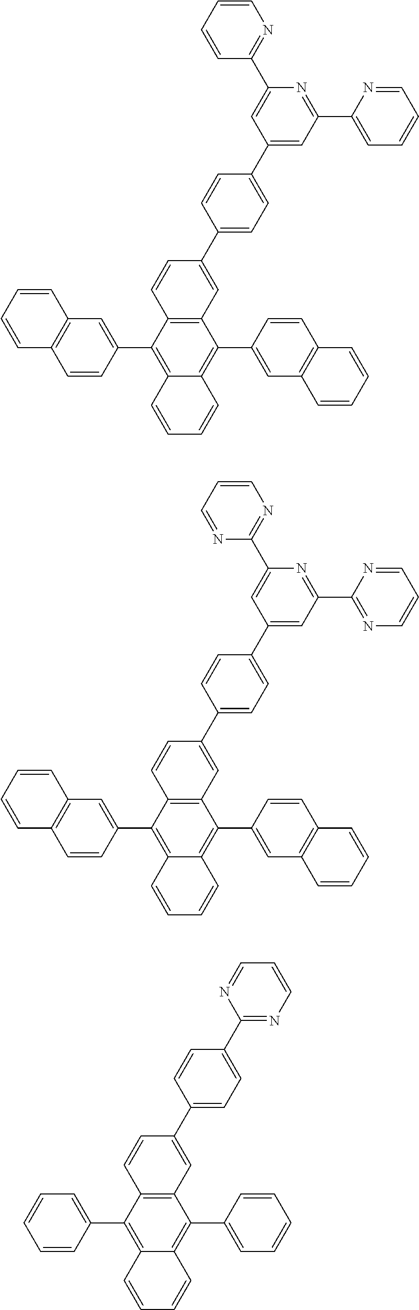 [SMILES: c1ccc(-c2cc(-c3ccc(-c4ccc5c(-c6ccc7ccccc7c6)c6ccccc6c(-c6ccc7ccccc7c6)c5c4)cc3)cc(-c3ccccn3)n2)nc1 ; c1cnc(-c2cc(-c3ccc(-c4ccc5c(-c6ccc7ccccc7c6)c6ccccc6c(-c6ccc7ccccc7c6)c5c4)cc3)cc(-c3ncccn3)n2)nc1 ; c1ccc(-c2c3ccccc3c(-c3ccccc3)c3cc(-c4ccc(-c5ncccn5)cc4)ccc23)cc1]

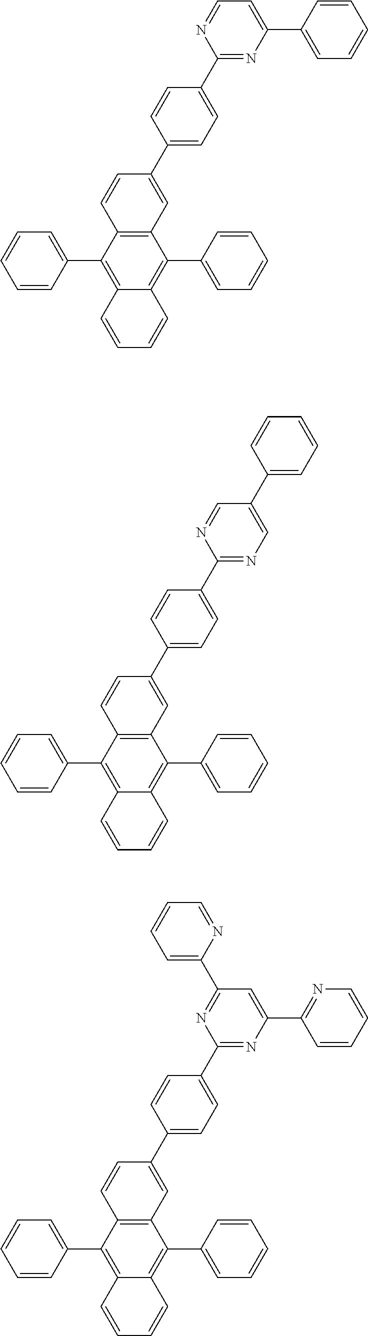 [SMILES: c1ccc(-c2ccnc(-c3ccc(-c4ccc5c(-c6ccccc6)c6ccccc6c(-c6ccccc6)c5c4)cc3)n2)cc1 ; c1ccc(-c2cnc(-c3ccc(-c4ccc5c(-c6ccccc6)c6ccccc6c(-c6ccccc6)c5c4)cc3)nc2)cc1 ; c1ccc(-c2c3ccccc3c(-c3ccccc3)c3cc(-c4ccc(-c5nc(-c6ccccn6)cc(-c6ccccn6)n5)cc4)ccc23)cc1]

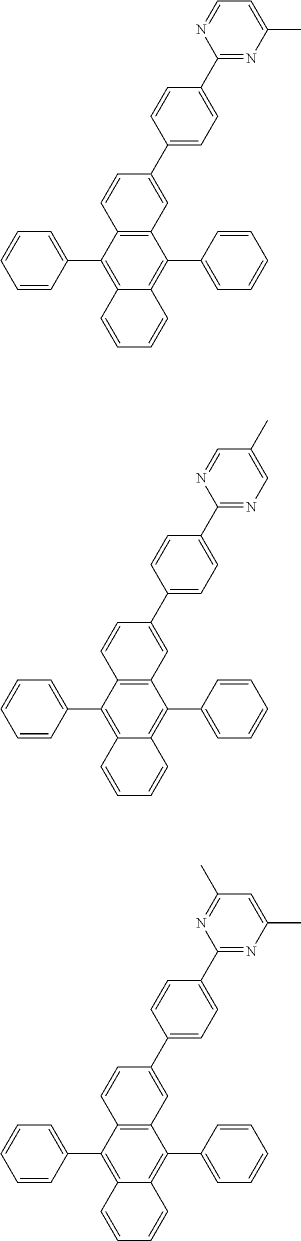 [SMILES: Cc1ccnc(-c2ccc(-c3ccc4c(-c5ccccc5)c5ccccc5c(-c5ccccc5)c4c3)cc2)n1 ; Cc1cnc(-c2ccc(-c3ccc4c(-c5ccccc5)c5ccccc5c(-c5ccccc5)c4c3)cc2)nc1 ; Cc1cc(C)nc(-c2ccc(-c3ccc4c(-c5ccccc5)c5ccccc5c(-c5ccccc5)c4c3)cc2)n1]

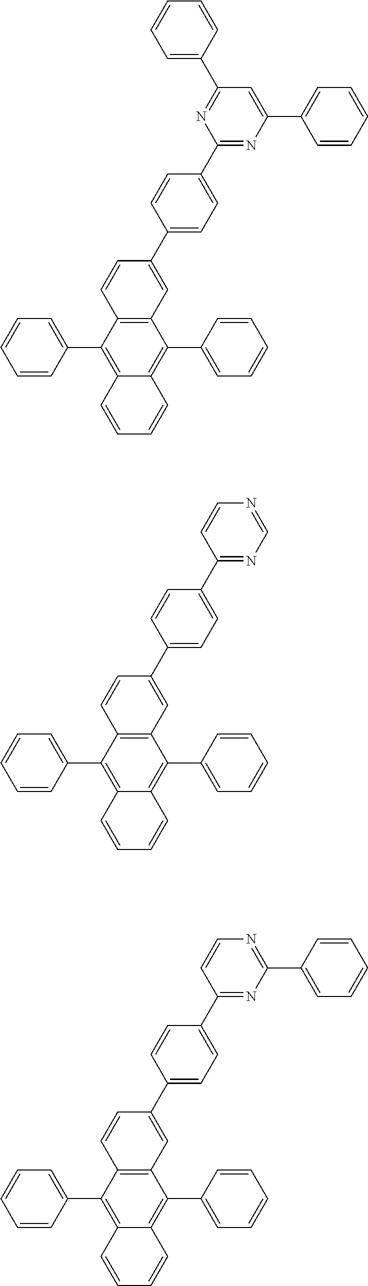 [SMILES: c1ccc(-c2cc(-c3ccccc3)nc(-c3ccc(-c4ccc5c(-c6ccccc6)c6ccccc6c(-c6ccccc6)c5c4)cc3)n2)cc1 ; c1ccc(-c2c3ccccc3c(-c3ccccc3)c3cc(-c4ccc(-c5ccncn5)cc4)ccc23)cc1 ; c1ccc(-c2nccc(-c3ccc(-c4ccc5c(-c6ccccc6)c6ccccc6c(-c6ccccc6)c5c4)cc3)n2)cc1]

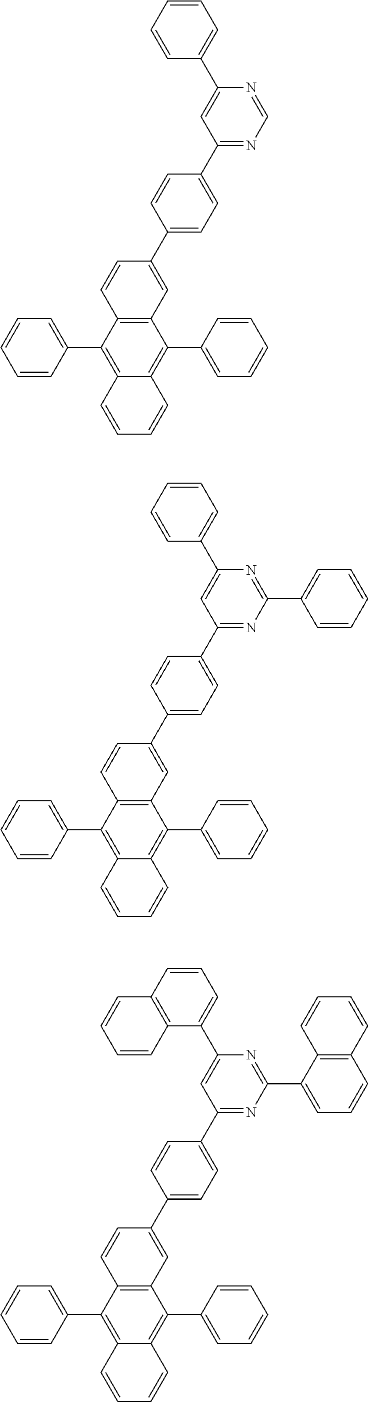 [SMILES: c1ccc(-c2cc(-c3ccc(-c4ccc5c(-c6ccccc6)c6ccccc6c(-c6ccccc6)c5c4)cc3)ncn2)cc1 ; c1ccc(-c2cc(-c3ccc(-c4ccc5c(-c6ccccc6)c6ccccc6c(-c6ccccc6)c5c4)cc3)nc(-c3ccccc3)n2)cc1 ; c1ccc(-c2c3ccccc3c(-c3ccccc3)c3cc(-c4ccc(-c5cc(-c6cccc7ccccc67)nc(-c6cccc7ccccc67)n5)cc4)ccc23)cc1]

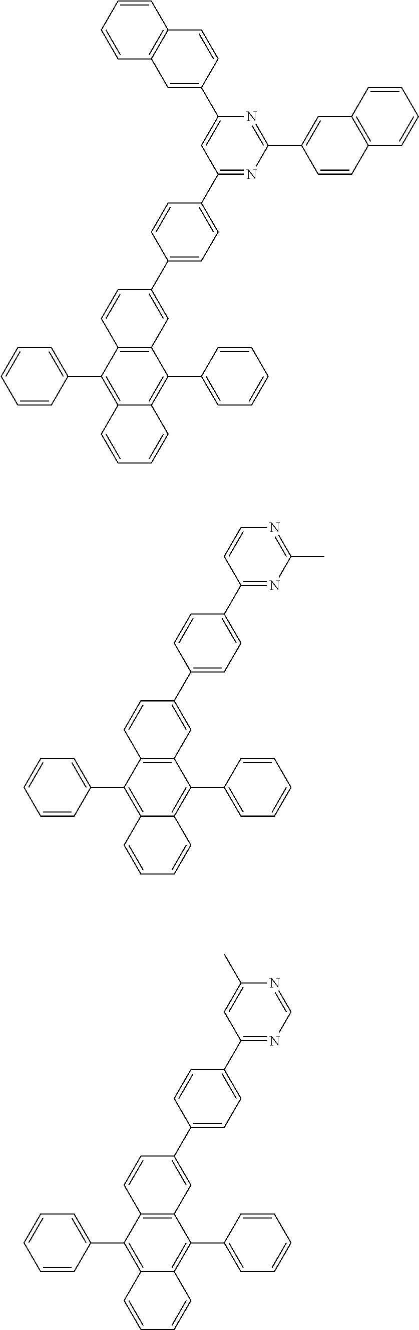 [SMILES: c1ccc(-c2c3ccccc3c(-c3ccccc3)c3cc(-c4ccc(-c5cc(-c6ccc7ccccc7c6)nc(-c6ccc7ccccc7c6)n5)cc4)ccc23)cc1 ; Cc1nccc(-c2ccc(-c3ccc4c(-c5ccccc5)c5ccccc5c(-c5ccccc5)c4c3)cc2)n1 ; Cc1cc(-c2ccc(-c3ccc4c(-c5ccccc5)c5ccccc5c(-c5ccccc5)c4c3)cc2)ncn1]

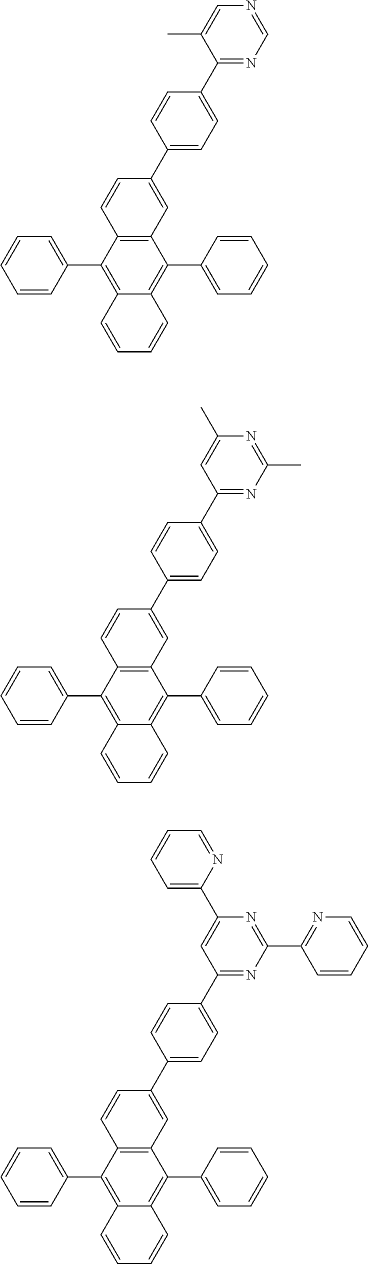 [SMILES: Cc1cncnc1-c1ccc(-c2ccc3c(-c4ccccc4)c4ccccc4c(-c4ccccc4)c3c2)cc1 ; Cc1cc(-c2ccc(-c3ccc4c(-c5ccccc5)c5ccccc5c(-c5ccccc5)c4c3)cc2)nc(C)n1 ; c1ccc(-c2c3ccccc3c(-c3ccccc3)c3cc(-c4ccc(-c5cc(-c6ccccn6)nc(-c6ccccn6)n5)cc4)ccc23)cc1]

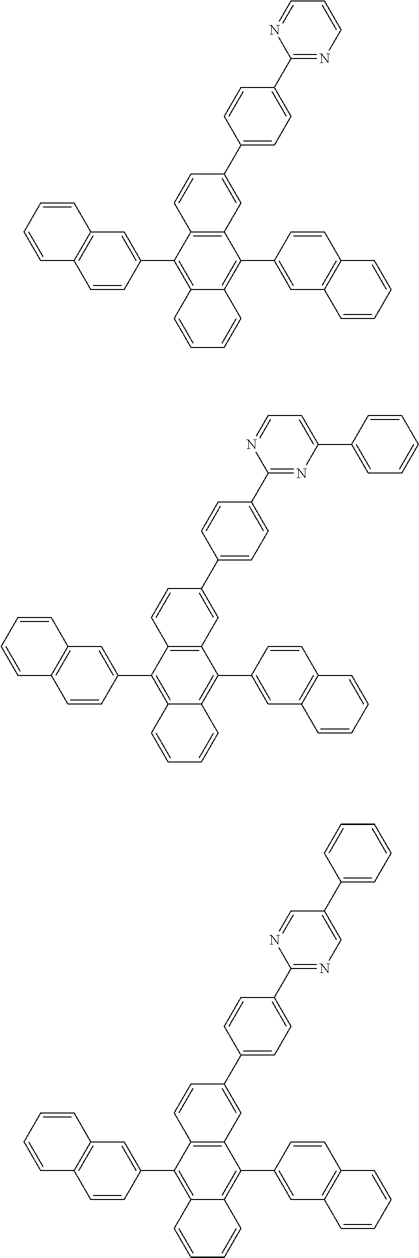 [SMILES: c1cnc(-c2ccc(-c3ccc4c(-c5ccc6ccccc6c5)c5ccccc5c(-c5ccc6ccccc6c5)c4c3)cc2)nc1 ; c1ccc(-c2ccnc(-c3ccc(-c4ccc5c(-c6ccc7ccccc7c6)c6ccccc6c(-c6ccc7ccccc7c6)c5c4)cc3)n2)cc1 ; c1ccc(-c2cnc(-c3ccc(-c4ccc5c(-c6ccc7ccccc7c6)c6ccccc6c(-c6ccc7ccccc7c6)c5c4)cc3)nc2)cc1]

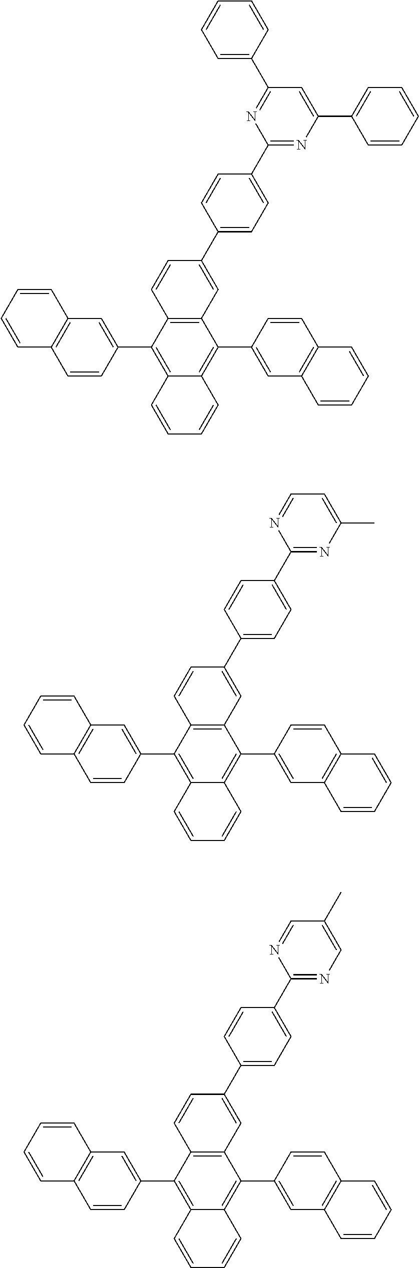 [SMILES: c1ccc(-c2cc(-c3ccccc3)nc(-c3ccc(-c4ccc5c(-c6ccc7ccccc7c6)c6ccccc6c(-c6ccc7ccccc7c6)c5c4)cc3)n2)cc1 ; Cc1ccnc(-c2ccc(-c3ccc4c(-c5ccc6ccccc6c5)c5ccccc5c(-c5ccc6ccccc6c5)c4c3)cc2)n1 ; Cc1cnc(-c2ccc(-c3ccc4c(-c5ccc6ccccc6c5)c5ccccc5c(-c5ccc6ccccc6c5)c4c3)cc2)nc1]

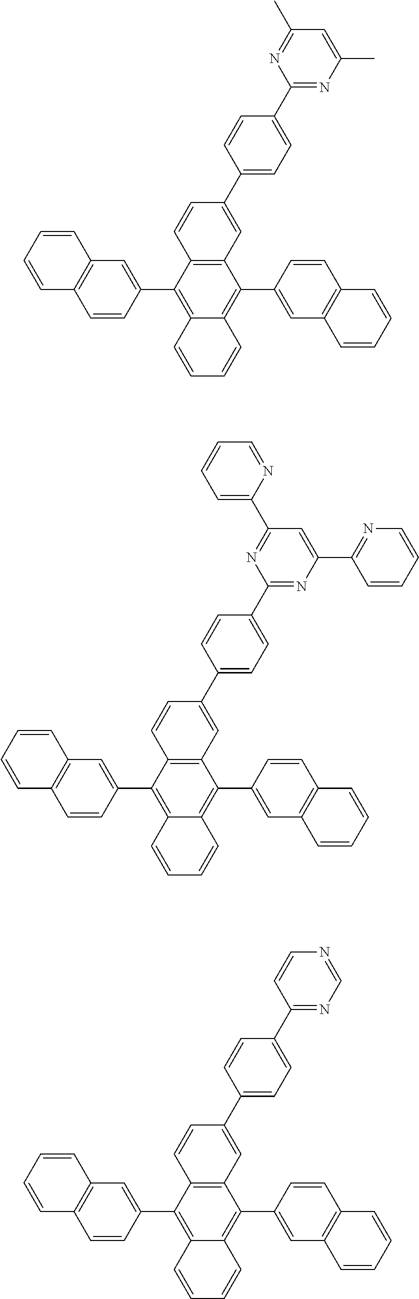 [SMILES: Cc1cc(C)nc(-c2ccc(-c3ccc4c(-c5ccc6ccccc6c5)c5ccccc5c(-c5ccc6ccccc6c5)c4c3)cc2)n1 ; c1ccc(-c2cc(-c3ccccn3)nc(-c3ccc(-c4ccc5c(-c6ccc7ccccc7c6)c6ccccc6c(-c6ccc7ccccc7c6)c5c4)cc3)n2)nc1 ; c1ccc2cc(-c3c4ccccc4c(-c4ccc5ccccc5c4)c4cc(-c5ccc(-c6ccncn6)cc5)ccc34)ccc2c1]

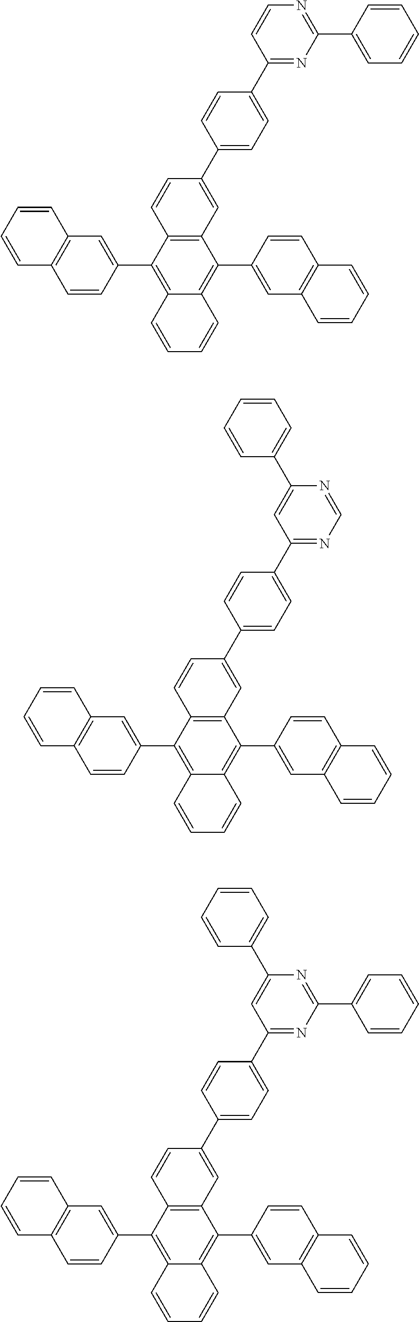 [SMILES: c1ccc(-c2nccc(-c3ccc(-c4ccc5c(-c6ccc7ccccc7c6)c6ccccc6c(-c6ccc7ccccc7c6)c5c4)cc3)n2)cc1 ; c1ccc(-c2cc(-c3ccc(-c4ccc5c(-c6ccc7ccccc7c6)c6ccccc6c(-c6ccc7ccccc7c6)c5c4)cc3)ncn2)cc1 ; c1ccc(-c2cc(-c3ccc(-c4ccc5c(-c6ccc7ccccc7c6)c6ccccc6c(-c6ccc7ccccc7c6)c5c4)cc3)nc(-c3ccccc3)n2)cc1]

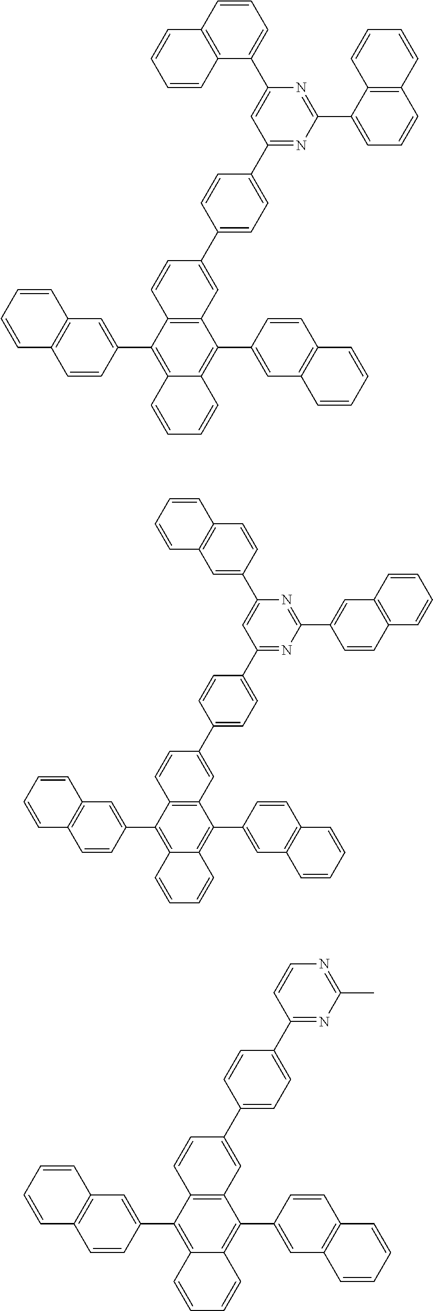 [SMILES: c1ccc2cc(-c3c4ccccc4c(-c4ccc5ccccc5c4)c4cc(-c5ccc(-c6cc(-c7cccc8ccccc78)nc(-c7cccc8ccccc78)n6)cc5)ccc34)ccc2c1 ; c1ccc2cc(-c3cc(-c4ccc(-c5ccc6c(-c7ccc8ccccc8c7)c7ccccc7c(-c7ccc8ccccc8c7)c6c5)cc4)nc(-c4ccc5ccccc5c4)n3)ccc2c1 ; Cc1nccc(-c2ccc(-c3ccc4c(-c5ccc6ccccc6c5)c5ccccc5c(-c5ccc6ccccc6c5)c4c3)cc2)n1]

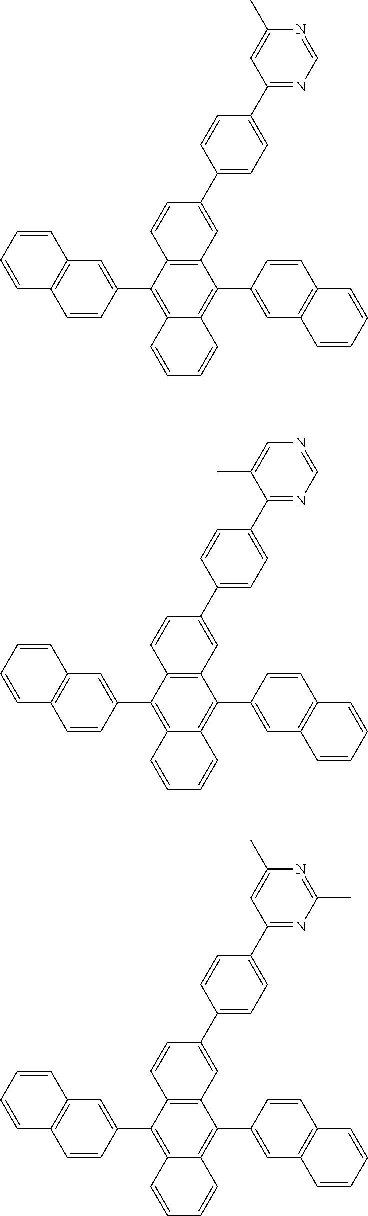 [SMILES: Cc1cc(-c2ccc(-c3ccc4c(-c5ccc6ccccc6c5)c5ccccc5c(-c5ccc6ccccc6c5)c4c3)cc2)ncn1 ; Cc1cncnc1-c1ccc(-c2ccc3c(-c4ccc5ccccc5c4)c4ccccc4c(-c4ccc5ccccc5c4)c3c2)cc1 ; Cc1cc(-c2ccc(-c3ccc4c(-c5ccc6ccccc6c5)c5ccccc5c(-c5ccc6ccccc6c5)c4c3)cc2)nc(C)n1]

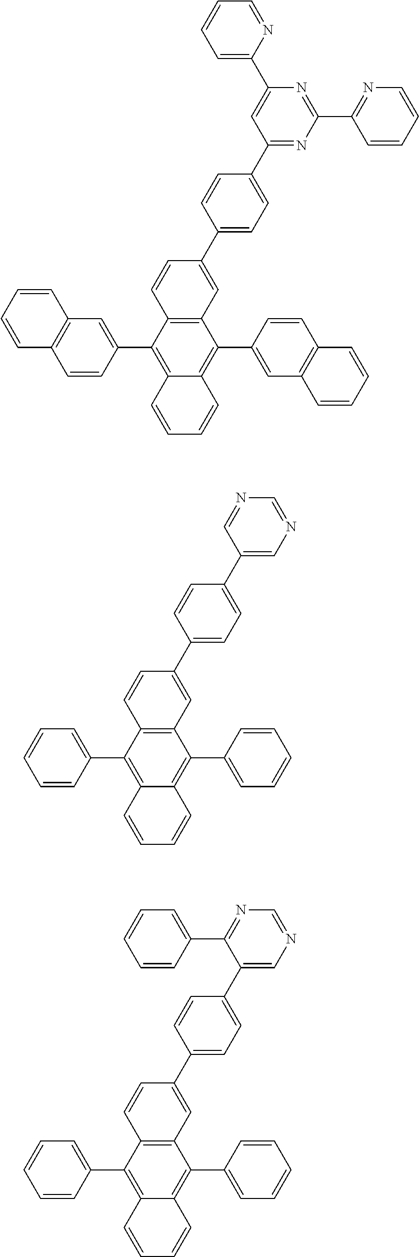 [SMILES: c1ccc(-c2cc(-c3ccc(-c4ccc5c(-c6ccc7ccccc7c6)c6ccccc6c(-c6ccc7ccccc7c6)c5c4)cc3)nc(-c3ccccn3)n2)nc1 ; c1ccc(-c2c3ccccc3c(-c3ccccc3)c3cc(-c4ccc(-c5cncnc5)cc4)ccc23)cc1 ; c1ccc(-c2ncncc2-c2ccc(-c3ccc4c(-c5ccccc5)c5ccccc5c(-c5ccccc5)c4c3)cc2)cc1]

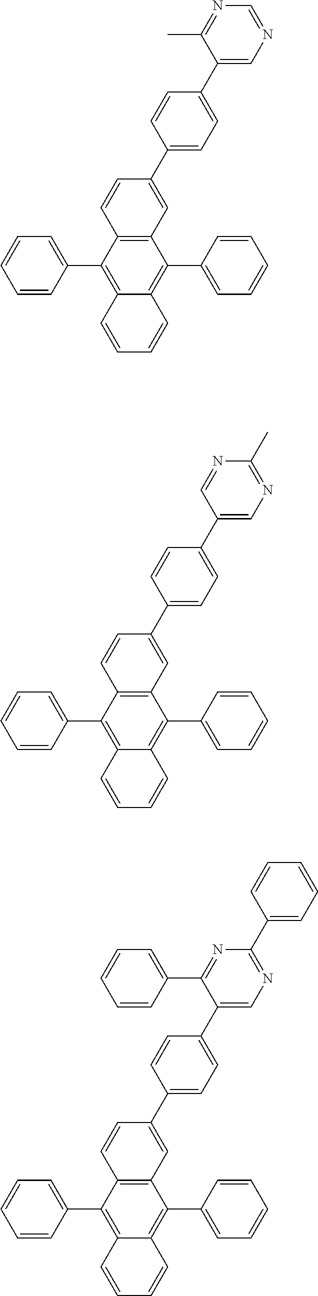 [SMILES: Cc1ncncc1-c1ccc(-c2ccc3c(-c4ccccc4)c4ccccc4c(-c4ccccc4)c3c2)cc1 ; Cc1ncc(-c2ccc(-c3ccc4c(-c5ccccc5)c5ccccc5c(-c5ccccc5)c4c3)cc2)cn1 ; c1ccc(-c2ncc(-c3ccc(-c4ccc5c(-c6ccccc6)c6ccccc6c(-c6ccccc6)c5c4)cc3)c(-c3ccccc3)n2)cc1]

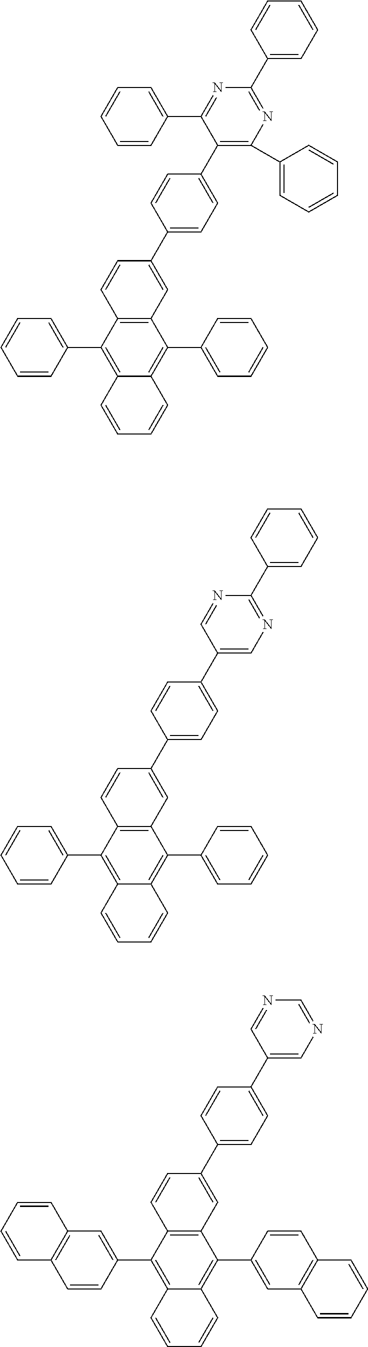 [SMILES: c1ccc(-c2nc(-c3ccccc3)c(-c3ccc(-c4ccc5c(-c6ccccc6)c6ccccc6c(-c6ccccc6)c5c4)cc3)c(-c3ccccc3)n2)cc1 ; c1ccc(-c2ncc(-c3ccc(-c4ccc5c(-c6ccccc6)c6ccccc6c(-c6ccccc6)c5c4)cc3)cn2)cc1 ; c1ccc2cc(-c3c4ccccc4c(-c4ccc5ccccc5c4)c4cc(-c5ccc(-c6cncnc6)cc5)ccc34)ccc2c1]

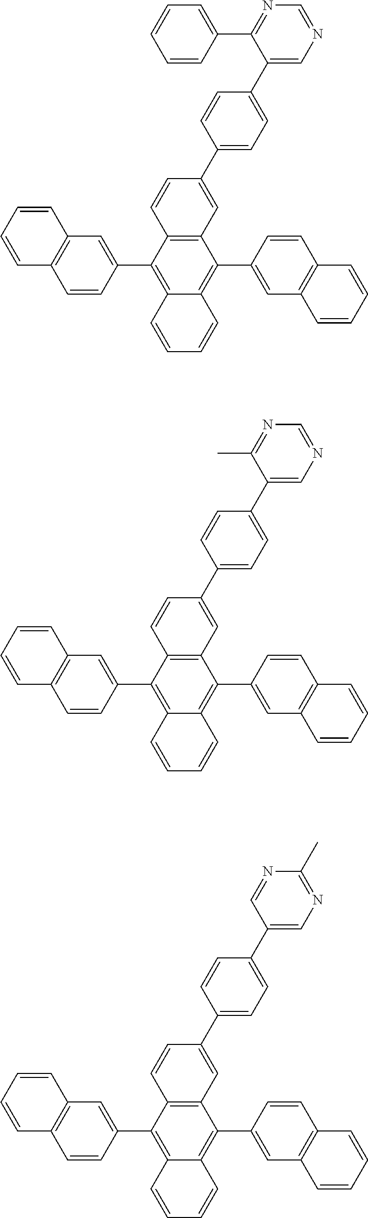 [SMILES: c1ccc(-c2ncncc2-c2ccc(-c3ccc4c(-c5ccc6ccccc6c5)c5ccccc5c(-c5ccc6ccccc6c5)c4c3)cc2)cc1 ; Cc1ncncc1-c1ccc(-c2ccc3c(-c4ccc5ccccc5c4)c4ccccc4c(-c4ccc5ccccc5c4)c3c2)cc1 ; Cc1ncc(-c2ccc(-c3ccc4c(-c5ccc6ccccc6c5)c5ccccc5c(-c5ccc6ccccc6c5)c4c3)cc2)cn1]

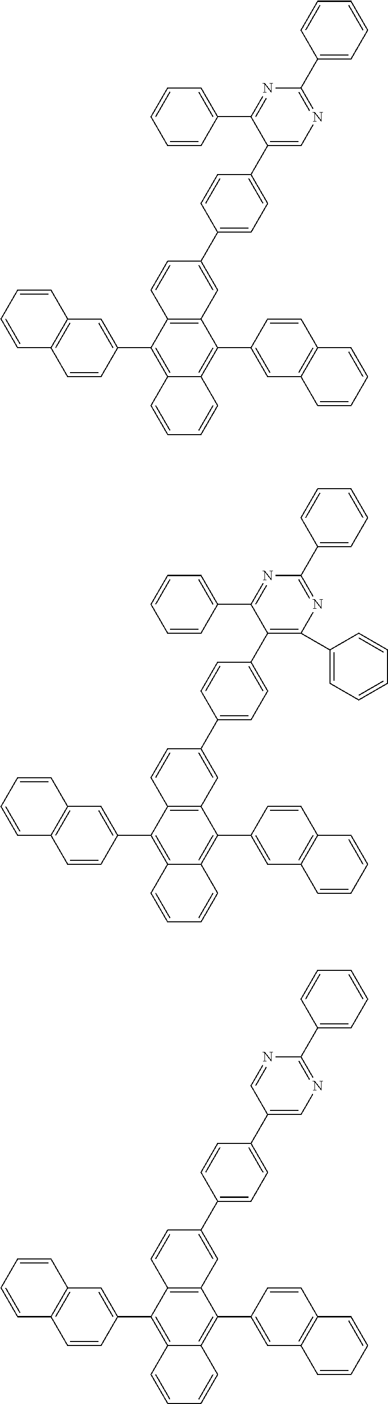 [SMILES: c1ccc(-c2ncc(-c3ccc(-c4ccc5c(-c6ccc7ccccc7c6)c6ccccc6c(-c6ccc7ccccc7c6)c5c4)cc3)c(-c3ccccc3)n2)cc1 ; c1ccc(-c2nc(-c3ccccc3)c(-c3ccc(-c4ccc5c(-c6ccc7ccccc7c6)c6ccccc6c(-c6ccc7ccccc7c6)c5c4)cc3)c(-c3ccccc3)n2)cc1 ; c1ccc(-c2ncc(-c3ccc(-c4ccc5c(-c6ccc7ccccc7c6)c6ccccc6c(-c6ccc7ccccc7c6)c5c4)cc3)cn2)cc1]

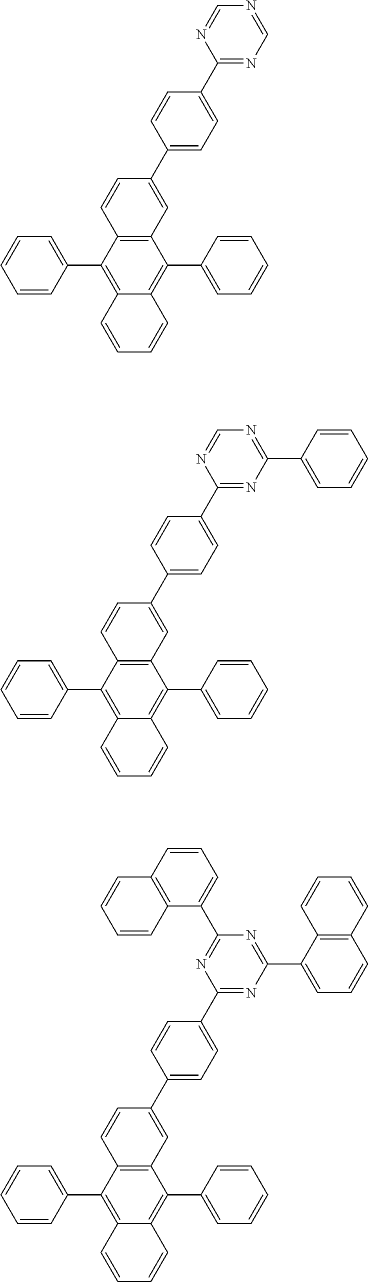 [SMILES: c1ccc(-c2c3ccccc3c(-c3ccccc3)c3cc(-c4ccc(-c5ncncn5)cc4)ccc23)cc1 ; c1ccc(-c2ncnc(-c3ccc(-c4ccc5c(-c6ccccc6)c6ccccc6c(-c6ccccc6)c5c4)cc3)n2)cc1 ; c1ccc(-c2c3ccccc3c(-c3ccccc3)c3cc(-c4ccc(-c5nc(-c6cccc7ccccc67)nc(-c6cccc7ccccc67)n5)cc4)ccc23)cc1]

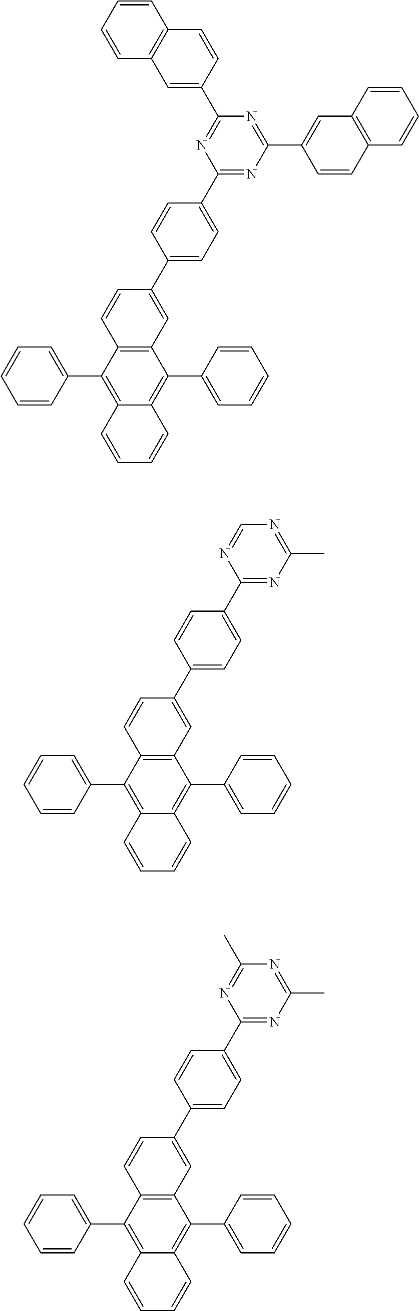 [SMILES: c1ccc(-c2c3ccccc3c(-c3ccccc3)c3cc(-c4ccc(-c5nc(-c6ccc7ccccc7c6)nc(-c6ccc7ccccc7c6)n5)cc4)ccc23)cc1 ; Cc1ncnc(-c2ccc(-c3ccc4c(-c5ccccc5)c5ccccc5c(-c5ccccc5)c4c3)cc2)n1 ; Cc1nc(C)nc(-c2ccc(-c3ccc4c(-c5ccccc5)c5ccccc5c(-c5ccccc5)c4c3)cc2)n1]

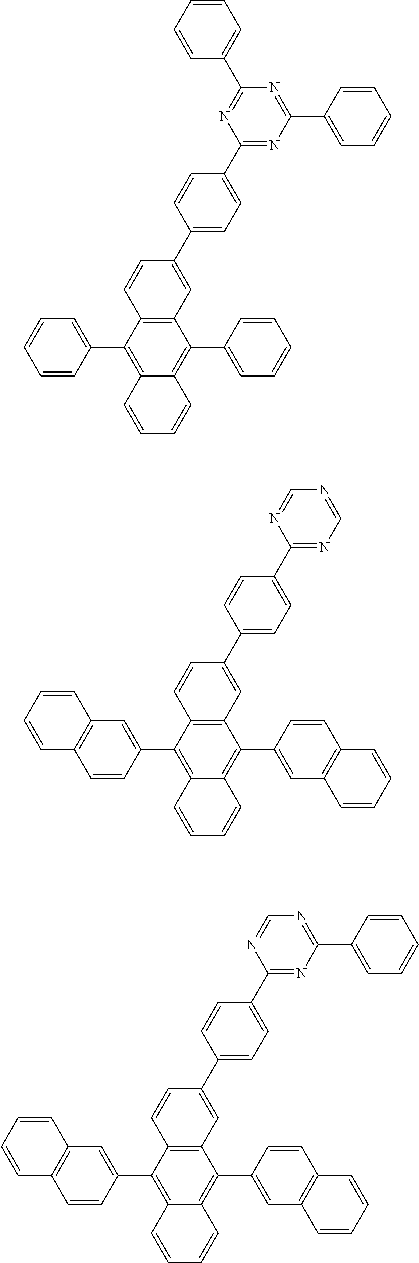 [SMILES: c1ccc(-c2nc(-c3ccccc3)nc(-c3ccc(-c4ccc5c(-c6ccccc6)c6ccccc6c(-c6ccccc6)c5c4)cc3)n2)cc1 ; c1ccc2cc(-c3c4ccccc4c(-c4ccc5ccccc5c4)c4cc(-c5ccc(-c6ncncn6)cc5)ccc34)ccc2c1 ; c1ccc(-c2ncnc(-c3ccc(-c4ccc5c(-c6ccc7ccccc7c6)c6ccccc6c(-c6ccc7ccccc7c6)c5c4)cc3)n2)cc1]

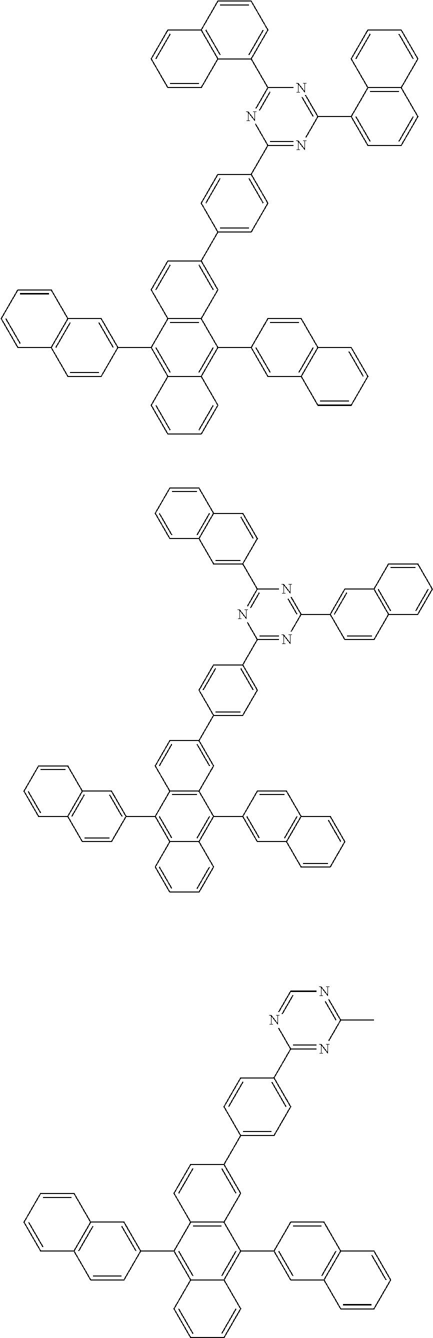 [SMILES: c1ccc2cc(-c3c4ccccc4c(-c4ccc5ccccc5c4)c4cc(-c5ccc(-c6nc(-c7cccc8ccccc78)nc(-c7cccc8ccccc78)n6)cc5)ccc34)ccc2c1 ; c1ccc2cc(-c3nc(-c4ccc(-c5ccc6c(-c7ccc8ccccc8c7)c7ccccc7c(-c7ccc8ccccc8c7)c6c5)cc4)nc(-c4ccc5ccccc5c4)n3)ccc2c1 ; Cc1ncnc(-c2ccc(-c3ccc4c(-c5ccc6ccccc6c5)c5ccccc5c(-c5ccc6ccccc6c5)c4c3)cc2)n1]

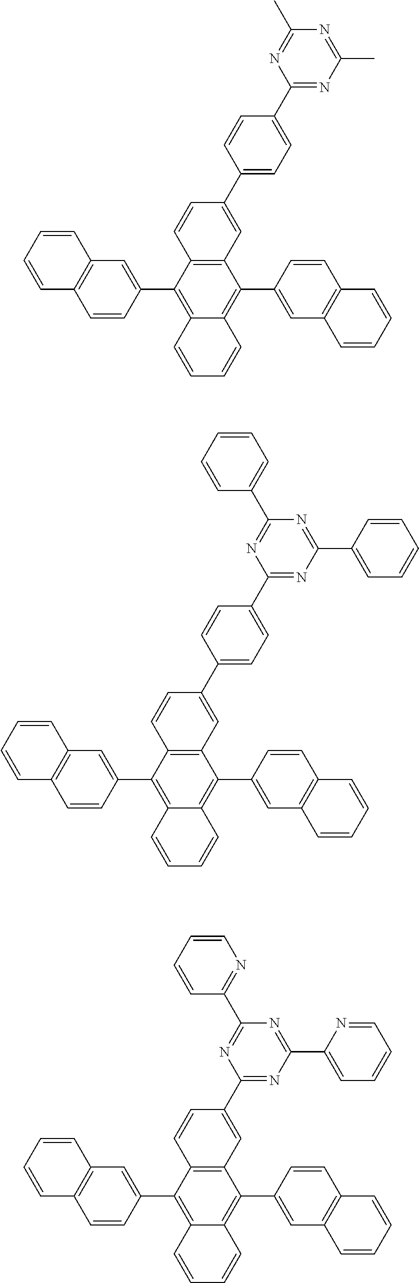 [SMILES: Cc1nc(C)nc(-c2ccc(-c3ccc4c(-c5ccc6ccccc6c5)c5ccccc5c(-c5ccc6ccccc6c5)c4c3)cc2)n1 ; c1ccc(-c2nc(-c3ccccc3)nc(-c3ccc(-c4ccc5c(-c6ccc7ccccc7c6)c6ccccc6c(-c6ccc7ccccc7c6)c5c4)cc3)n2)cc1 ; c1ccc(-c2nc(-c3ccc4c(-c5ccc6ccccc6c5)c5ccccc5c(-c5ccc6ccccc6c5)c4c3)nc(-c3ccccn3)n2)nc1]

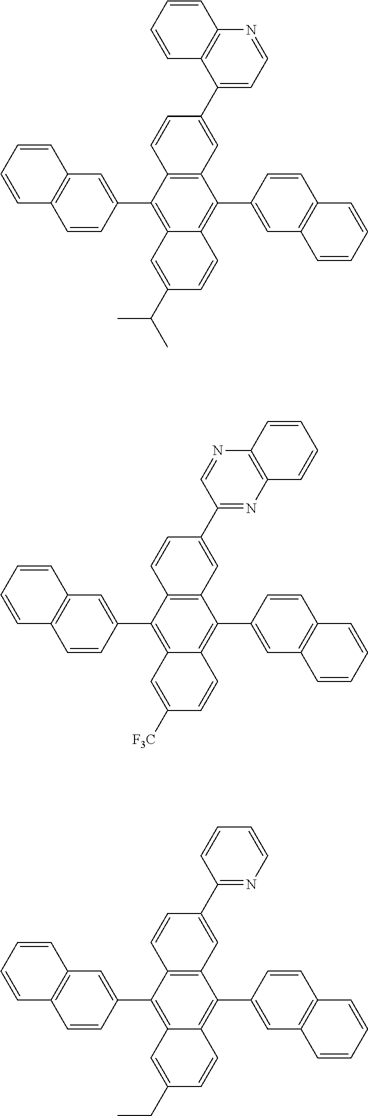 [SMILES: CC(C)c1ccc2c(-c3ccc4ccccc4c3)c3cc(-c4ccnc5ccccc45)ccc3c(-c3ccc4ccccc4c3)c2c1 ; FC(F)(F)c1ccc2c(-c3ccc4ccccc4c3)c3cc(-c4cnc5ccccc5n4)ccc3c(-c3ccc4ccccc4c3)c2c1 ; CCc1ccc2c(-c3ccc4ccccc4c3)c3cc(-c4ccccn4)ccc3c(-c3ccc4ccccc4c3)c2c1]

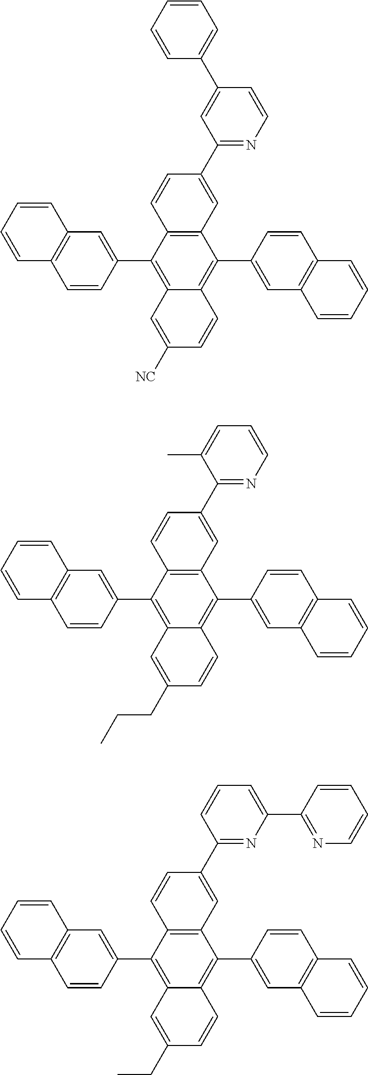 [SMILES: N#Cc1ccc2c(-c3ccc4ccccc4c3)c3cc(-c4cc(-c5ccccc5)ccn4)ccc3c(-c3ccc4ccccc4c3)c2c1 ; CCCc1ccc2c(-c3ccc4ccccc4c3)c3cc(-c4ncccc4C)ccc3c(-c3ccc4ccccc4c3)c2c1 ; CCc1ccc2c(-c3ccc4ccccc4c3)c3cc(-c4cccc(-c5ccccn5)n4)ccc3c(-c3ccc4ccccc4c3)c2c1]

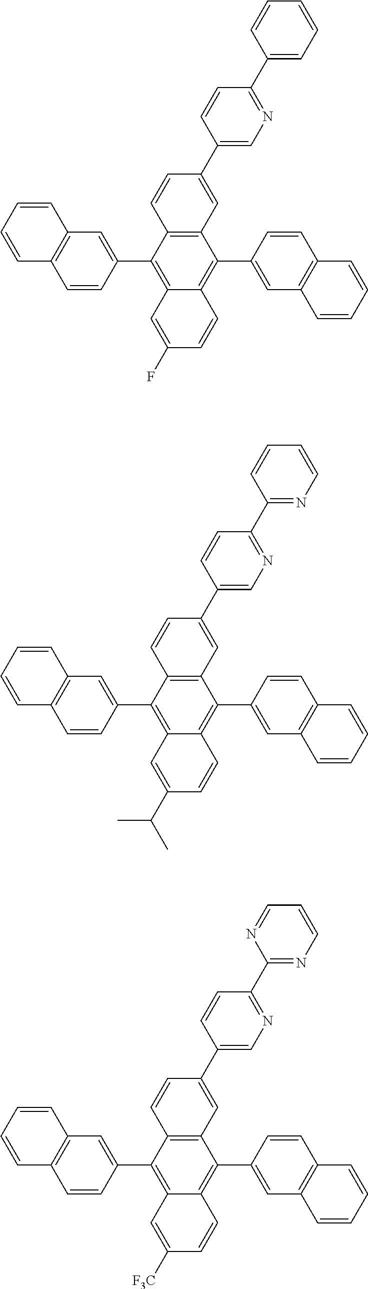 [SMILES: Fc1ccc2c(-c3ccc4ccccc4c3)c3cc(-c4ccc(-c5ccccc5)nc4)ccc3c(-c3ccc4ccccc4c3)c2c1 ; CC(C)c1ccc2c(-c3ccc4ccccc4c3)c3cc(-c4ccc(-c5ccccn5)nc4)ccc3c(-c3ccc4ccccc4c3)c2c1 ; FC(F)(F)c1ccc2c(-c3ccc4ccccc4c3)c3cc(-c4ccc(-c5ncccn5)nc4)ccc3c(-c3ccc4ccccc4c3)c2c1]

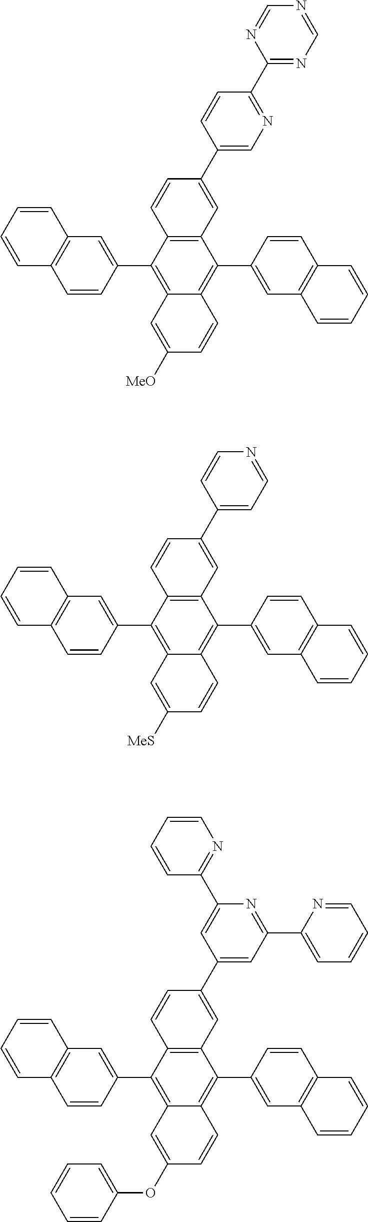 [SMILES: COc1ccc2c(-c3ccc4ccccc4c3)c3cc(-c4ccc(-c5ncncn5)nc4)ccc3c(-c3ccc4ccccc4c3)c2c1 ; CSc1ccc2c(-c3ccc4ccccc4c3)c3cc(-c4ccncc4)ccc3c(-c3ccc4ccccc4c3)c2c1 ; c1ccc(Oc2ccc3c(-c4ccc5ccccc5c4)c4cc(-c5cc(-c6ccccn6)nc(-c6ccccn6)c5)ccc4c(-c4ccc5ccccc5c4)c3c2)cc1]

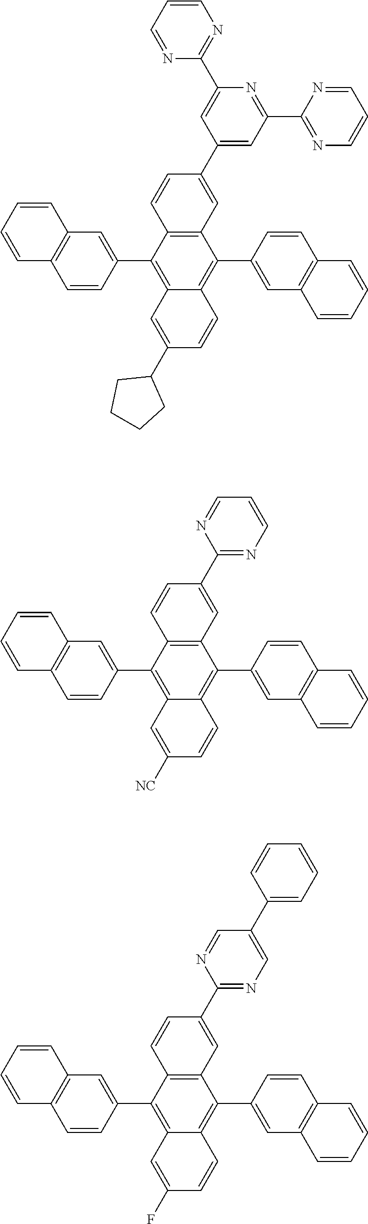 [SMILES: c1cnc(-c2cc(-c3ccc4c(-c5ccc6ccccc6c5)c5cc(C6CCCC6)ccc5c(-c5ccc6ccccc6c5)c4c3)cc(-c3ncccn3)n2)nc1 ; N#Cc1ccc2c(-c3ccc4ccccc4c3)c3cc(-c4ncccn4)ccc3c(-c3ccc4ccccc4c3)c2c1 ; Fc1ccc2c(-c3ccc4ccccc4c3)c3cc(-c4ncc(-c5ccccc5)cn4)ccc3c(-c3ccc4ccccc4c3)c2c1]

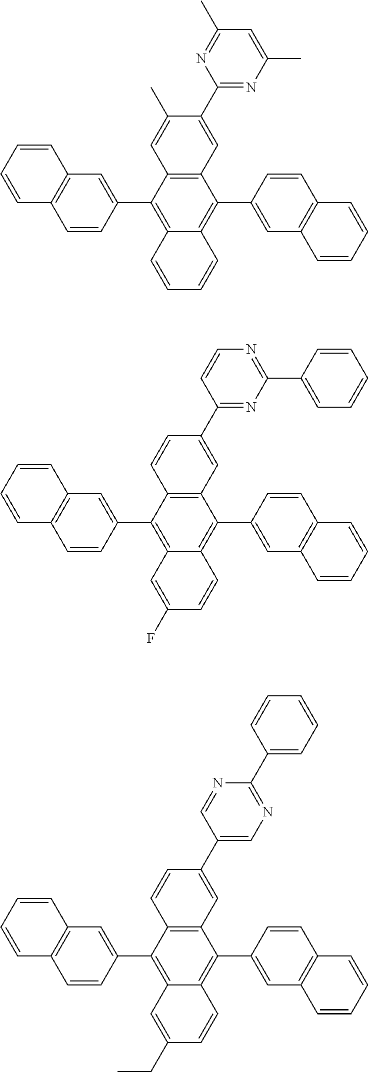 [SMILES: Cc1cc(C)nc(-c2cc3c(-c4ccc5ccccc5c4)c4ccccc4c(-c4ccc5ccccc5c4)c3cc2C)n1 ; Fc1ccc2c(-c3ccc4ccccc4c3)c3cc(-c4ccnc(-c5ccccc5)n4)ccc3c(-c3ccc4ccccc4c3)c2c1 ; CCc1ccc2c(-c3ccc4ccccc4c3)c3cc(-c4cnc(-c5ccccc5)nc4)ccc3c(-c3ccc4ccccc4c3)c2c1]

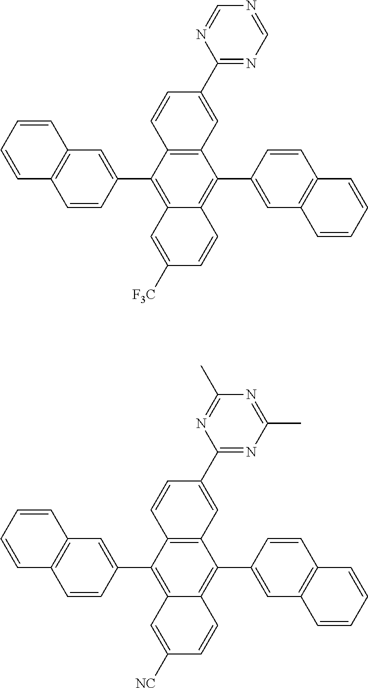 [SMILES: FC(F)(F)c1ccc2c(-c3ccc4ccccc4c3)c3cc(-c4ncncn4)ccc3c(-c3ccc4ccccc4c3)c2c1 ; Cc1nc(C)nc(-c2ccc3c(-c4ccc5ccccc5c4)c4cc(C#N)ccc4c(-c4ccc5ccccc5c4)c3c2)n1]

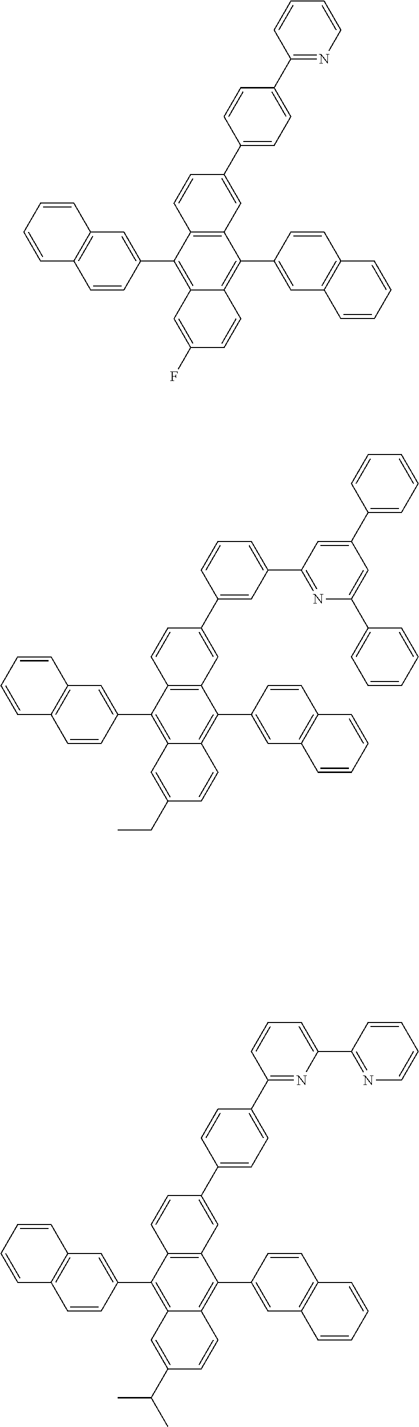 [SMILES: Fc1ccc2c(-c3ccc4ccccc4c3)c3cc(-c4ccc(-c5ccccn5)cc4)ccc3c(-c3ccc4ccccc4c3)c2c1 ; CCc1ccc2c(-c3ccc4ccccc4c3)c3cc(-c4cccc(-c5cc(-c6ccccc6)cc(-c6ccccc6)n5)c4)ccc3c(-c3ccc4ccccc4c3)c2c1 ; CC(C)c1ccc2c(-c3ccc4ccccc4c3)c3cc(-c4ccc(-c5cccc(-c6ccccn6)n5)cc4)ccc3c(-c3ccc4ccccc4c3)c2c1]

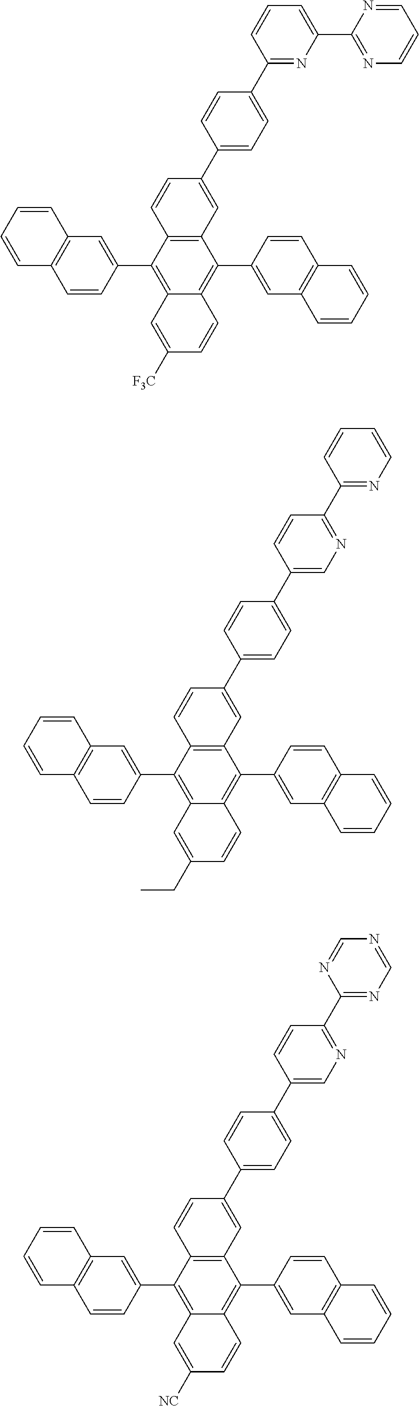 [SMILES: FC(F)(F)c1ccc2c(-c3ccc4ccccc4c3)c3cc(-c4ccc(-c5cccc(-c6ncccn6)n5)cc4)ccc3c(-c3ccc4ccccc4c3)c2c1 ; CCc1ccc2c(-c3ccc4ccccc4c3)c3cc(-c4ccc(-c5ccc(-c6ccccn6)nc5)cc4)ccc3c(-c3ccc4ccccc4c3)c2c1 ; N#Cc1ccc2c(-c3ccc4ccccc4c3)c3cc(-c4ccc(-c5ccc(-c6ncncn6)nc5)cc4)ccc3c(-c3ccc4ccccc4c3)c2c1]

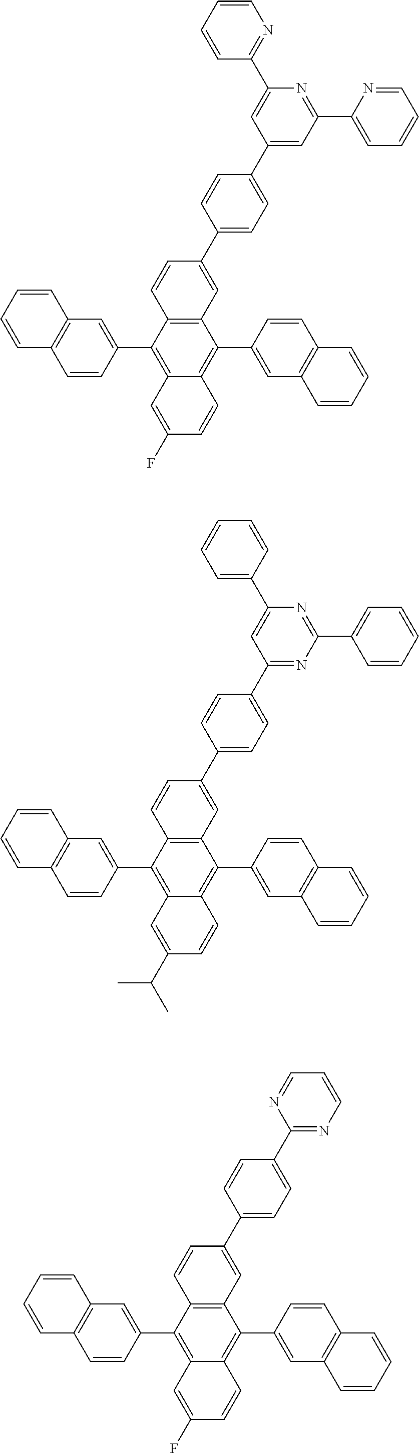 [SMILES: Fc1ccc2c(-c3ccc4ccccc4c3)c3cc(-c4ccc(-c5cc(-c6ccccn6)nc(-c6ccccn6)c5)cc4)ccc3c(-c3ccc4ccccc4c3)c2c1 ; CC(C)c1ccc2c(-c3ccc4ccccc4c3)c3cc(-c4ccc(-c5cc(-c6ccccc6)nc(-c6ccccc6)n5)cc4)ccc3c(-c3ccc4ccccc4c3)c2c1 ; Fc1ccc2c(-c3ccc4ccccc4c3)c3cc(-c4ccc(-c5ncccn5)cc4)ccc3c(-c3ccc4ccccc4c3)c2c1]

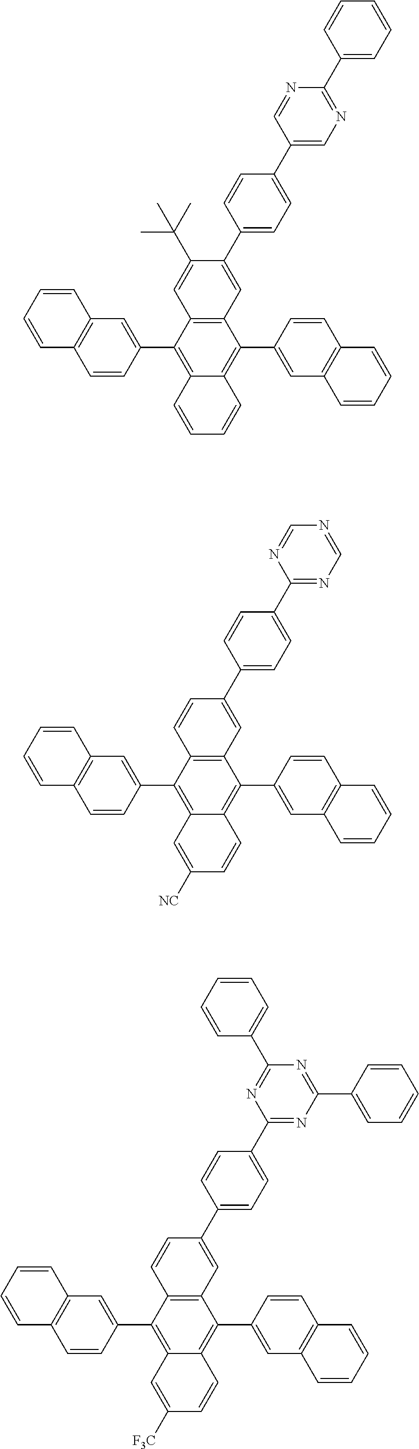 [SMILES: CC(C)(C)c1cc2c(-c3ccc4ccccc4c3)c3ccccc3c(-c3ccc4ccccc4c3)c2cc1-c1ccc(-c2cnc(-c3ccccc3)nc2)cc1 ; N#Cc1ccc2c(-c3ccc4ccccc4c3)c3cc(-c4ccc(-c5ncncn5)cc4)ccc3c(-c3ccc4ccccc4c3)c2c1 ; FC(F)(F)c1ccc2c(-c3ccc4ccccc4c3)c3cc(-c4ccc(-c5nc(-c6ccccc6)nc(-c6ccccc6)n5)cc4)ccc3c(-c3ccc4ccccc4c3)c2c1]

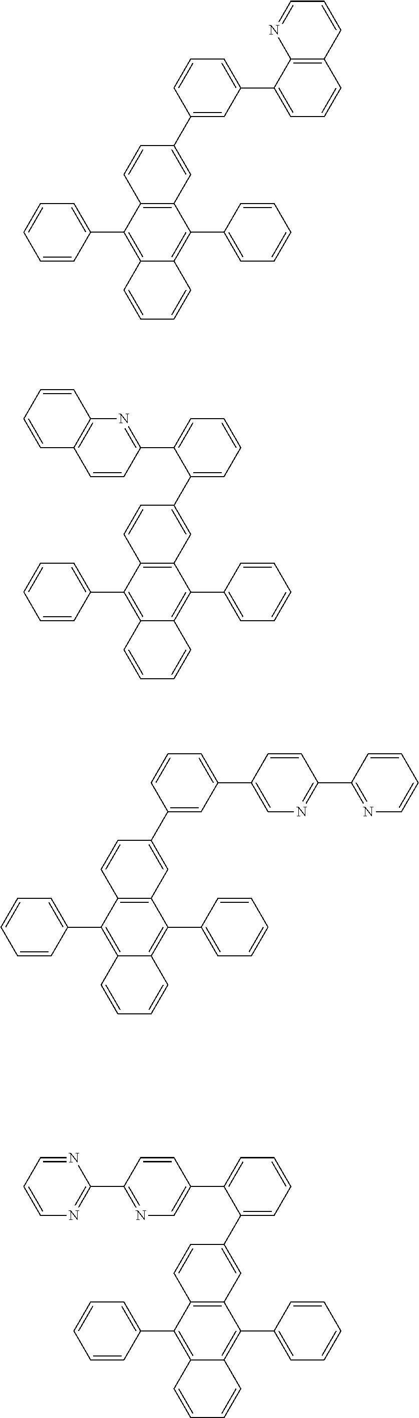 [SMILES: c1ccc(-c2c3ccccc3c(-c3ccccc3)c3cc(-c4cccc(-c5cccc6cccnc56)c4)ccc23)cc1 ; c1ccc(-c2c3ccccc3c(-c3ccccc3)c3cc(-c4ccccc4-c4ccc5ccccc5n4)ccc23)cc1 ; c1ccc(-c2c3ccccc3c(-c3ccccc3)c3cc(-c4cccc(-c5ccc(-c6ccccn6)nc5)c4)ccc23)cc1 ; c1ccc(-c2c3ccccc3c(-c3ccccc3)c3cc(-c4ccccc4-c4ccc(-c5ncccn5)nc4)ccc23)cc1]

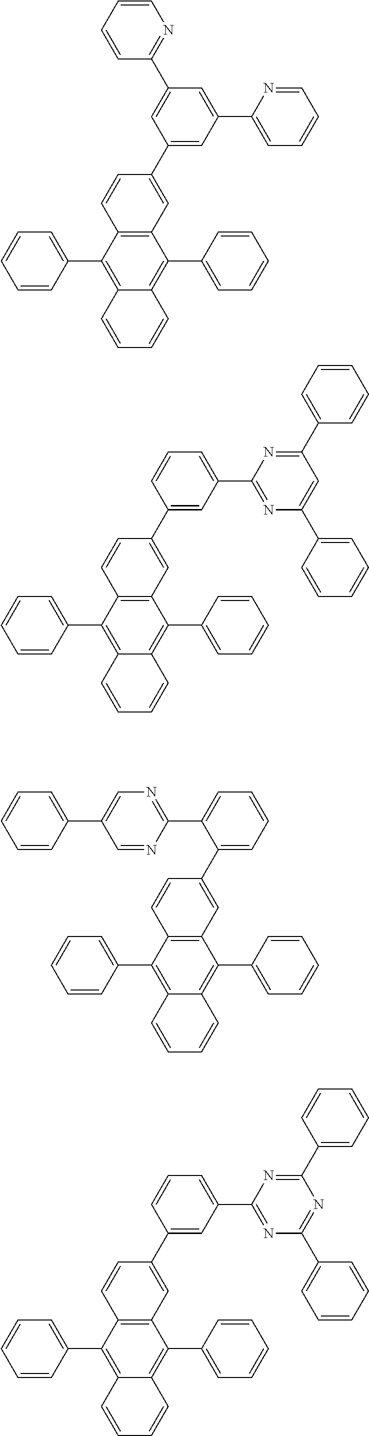 [SMILES: c1ccc(-c2c3ccccc3c(-c3ccccc3)c3cc(-c4cc(-c5ccccn5)cc(-c5ccccn5)c4)ccc23)cc1 ; c1ccc(-c2cc(-c3ccccc3)nc(-c3cccc(-c4ccc5c(-c6ccccc6)c6ccccc6c(-c6ccccc6)c5c4)c3)n2)cc1 ; c1ccc(-c2cnc(-c3ccccc3-c3ccc4c(-c5ccccc5)c5ccccc5c(-c5ccccc5)c4c3)nc2)cc1 ; c1ccc(-c2nc(-c3ccccc3)nc(-c3cccc(-c4ccc5c(-c6ccccc6)c6ccccc6c(-c6ccccc6)c5c4)c3)n2)cc1]

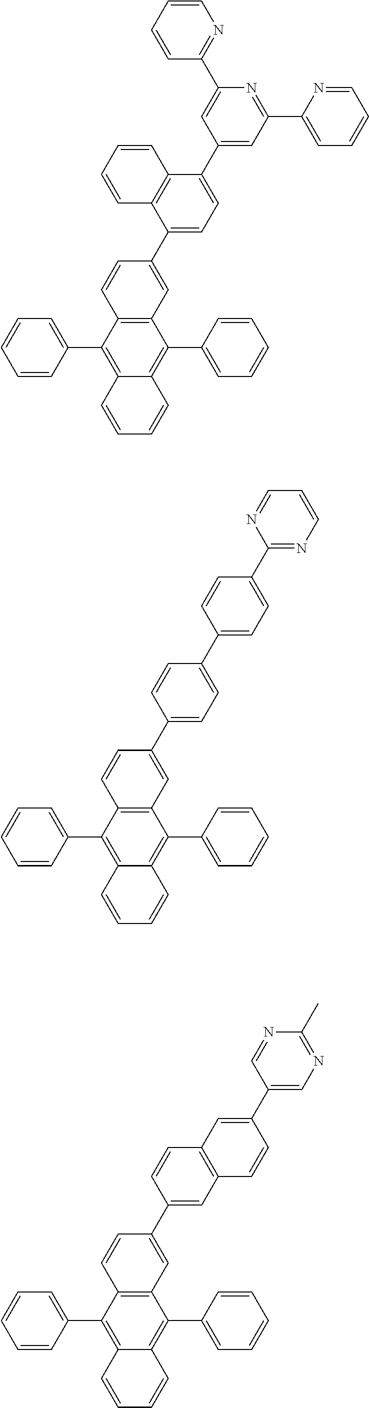 [SMILES: c1ccc(-c2c3ccccc3c(-c3ccccc3)c3cc(-c4ccc(-c5cc(-c6ccccn6)nc(-c6ccccn6)c5)c5ccccc45)ccc23)cc1 ; c1ccc(-c2c3ccccc3c(-c3ccccc3)c3cc(-c4ccc(-c5ccc(-c6ncccn6)cc5)cc4)ccc23)cc1 ; Cc1ncc(-c2ccc3cc(-c4ccc5c(-c6ccccc6)c6ccccc6c(-c6ccccc6)c5c4)ccc3c2)cn1]

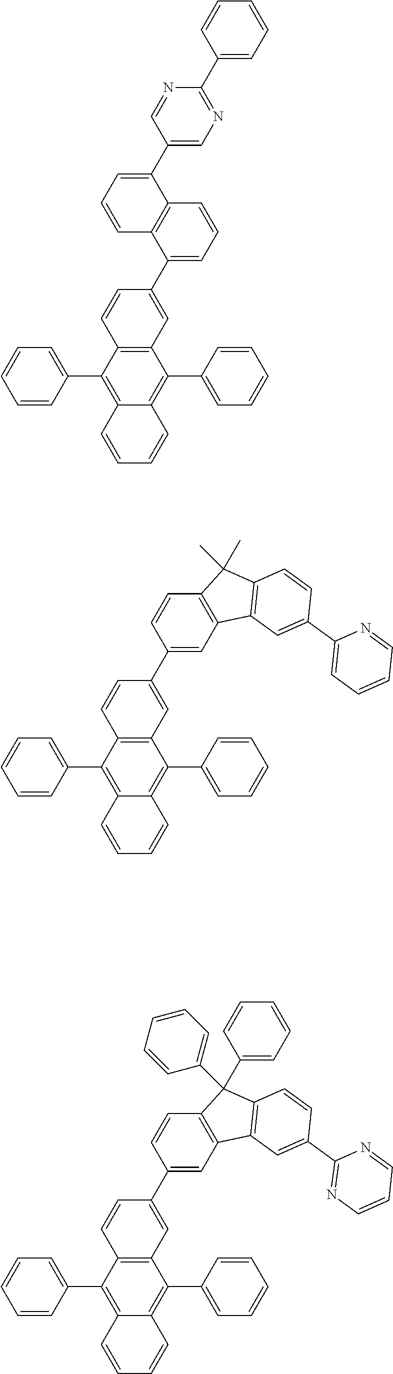 [SMILES: c1ccc(-c2ncc(-c3cccc4c(-c5ccc6c(-c7ccccc7)c7ccccc7c(-c7ccccc7)c6c5)cccc34)cn2)cc1 ; CC1(C)c2ccc(-c3ccc4c(-c5ccccc5)c5ccccc5c(-c5ccccc5)c4c3)cc2-c2cc(-c3ccccn3)ccc21 ; c1ccc(-c2c3ccccc3c(-c3ccccc3)c3cc(-c4ccc5c(c4)-c4cc(-c6ncccn6)ccc4C5(c4ccccc4)c4ccccc4)ccc23)cc1]

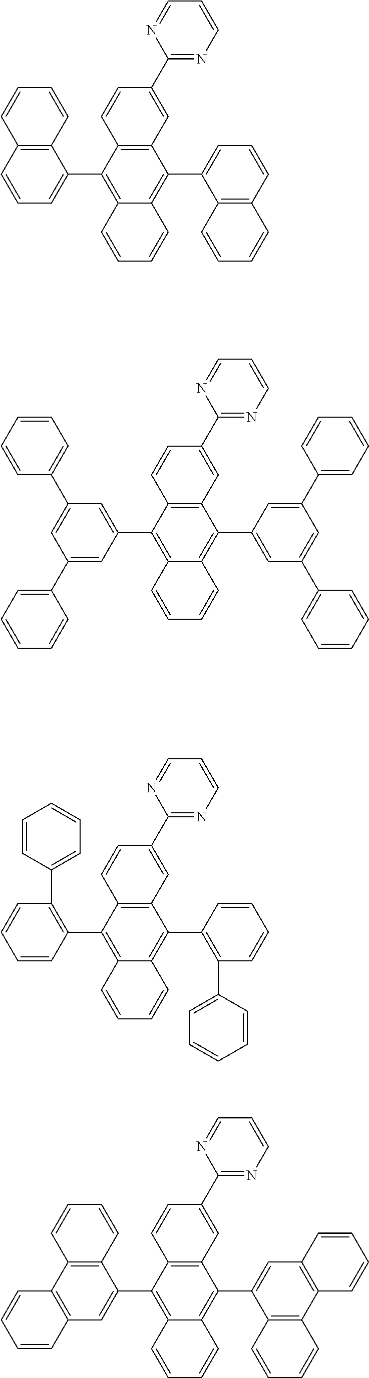 [SMILES: c1cnc(-c2ccc3c(-c4cccc5ccccc45)c4ccccc4c(-c4cccc5ccccc45)c3c2)nc1 ; c1ccc(-c2cc(-c3ccccc3)cc(-c3c4ccccc4c(-c4cc(-c5ccccc5)cc(-c5ccccc5)c4)c4cc(-c5ncccn5)ccc34)c2)cc1 ; c1ccc(-c2ccccc2-c2c3ccccc3c(-c3ccccc3-c3ccccc3)c3cc(-c4ncccn4)ccc23)cc1 ; c1cnc(-c2ccc3c(-c4cc5ccccc5c5ccccc45)c4ccccc4c(-c4cc5ccccc5c5ccccc45)c3c2)nc1]

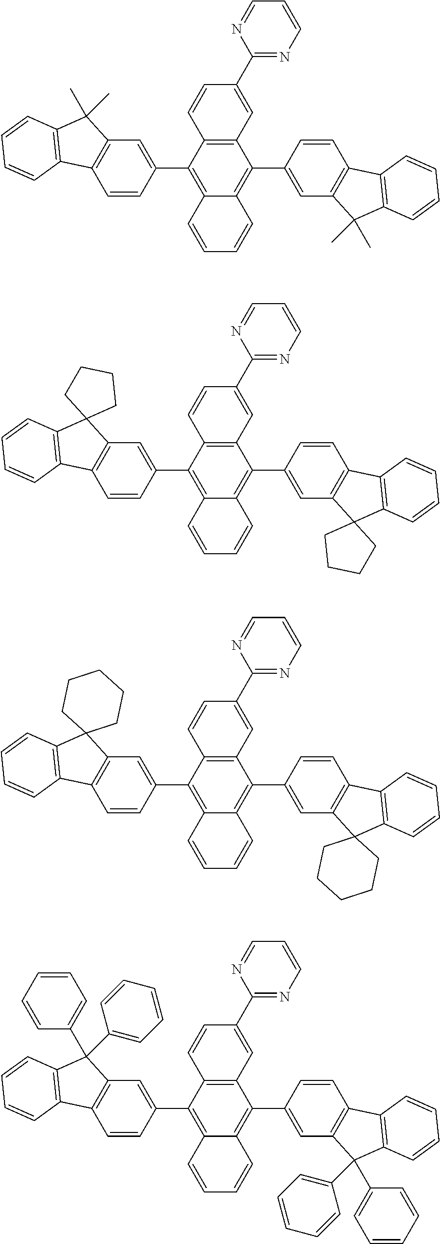 [SMILES: CC1(C)c2ccccc2-c2ccc(-c3c4ccccc4c(-c4ccc5c(c4)C(C)(C)c4ccccc4-5)c4cc(-c5ncccn5)ccc34)cc21 ; c1cnc(-c2ccc3c(-c4ccc5c(c4)C4(CCCC4)c4ccccc4-5)c4ccccc4c(-c4ccc5c(c4)C4(CCCC4)c4ccccc4-5)c3c2)nc1 ; c1cnc(-c2ccc3c(-c4ccc5c(c4)C4(CCCCC4)c4ccccc4-5)c4ccccc4c(-c4ccc5c(c4)C4(CCCCC4)c4ccccc4-5)c3c2)nc1 ; c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ncccn6)ccc45)cc32)cc1]

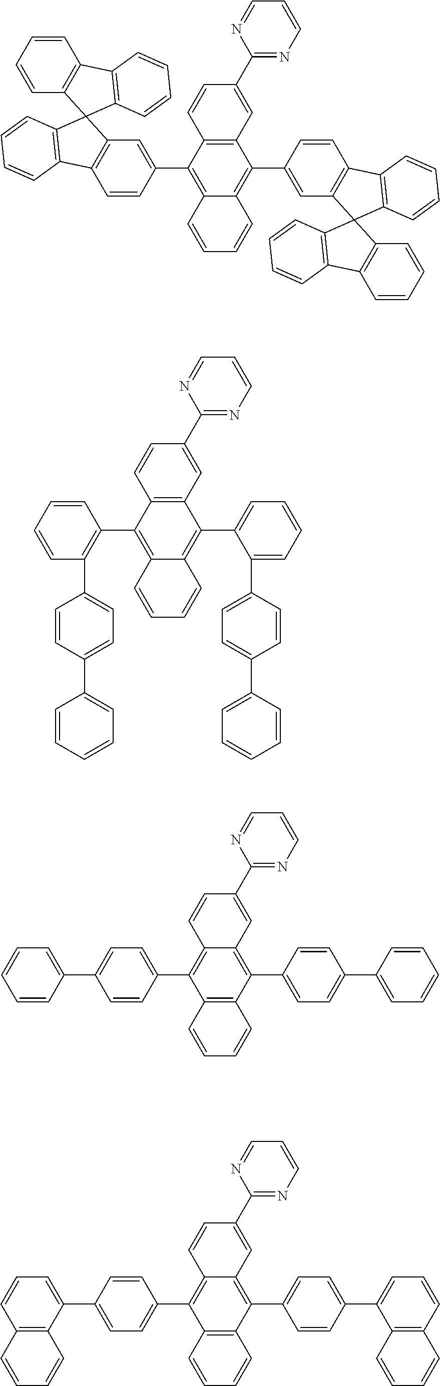 [SMILES: c1cnc(-c2ccc3c(-c4ccc5c(c4)C4(c6ccccc6-c6ccccc64)c4ccccc4-5)c4ccccc4c(-c4ccc5c(c4)C4(c6ccccc6-c6ccccc64)c4ccccc4-5)c3c2)nc1 ; c1ccc(-c2ccc(-c3ccccc3-c3c4ccccc4c(-c4ccccc4-c4ccc(-c5ccccc5)cc4)c4cc(-c5ncccn5)ccc34)cc2)cc1 ; c1ccc(-c2ccc(-c3c4ccccc4c(-c4ccc(-c5ccccc5)cc4)c4cc(-c5ncccn5)ccc34)cc2)cc1 ; c1cnc(-c2ccc3c(-c4ccc(-c5cccc6ccccc56)cc4)c4ccccc4c(-c4ccc(-c5cccc6ccccc56)cc4)c3c2)nc1]

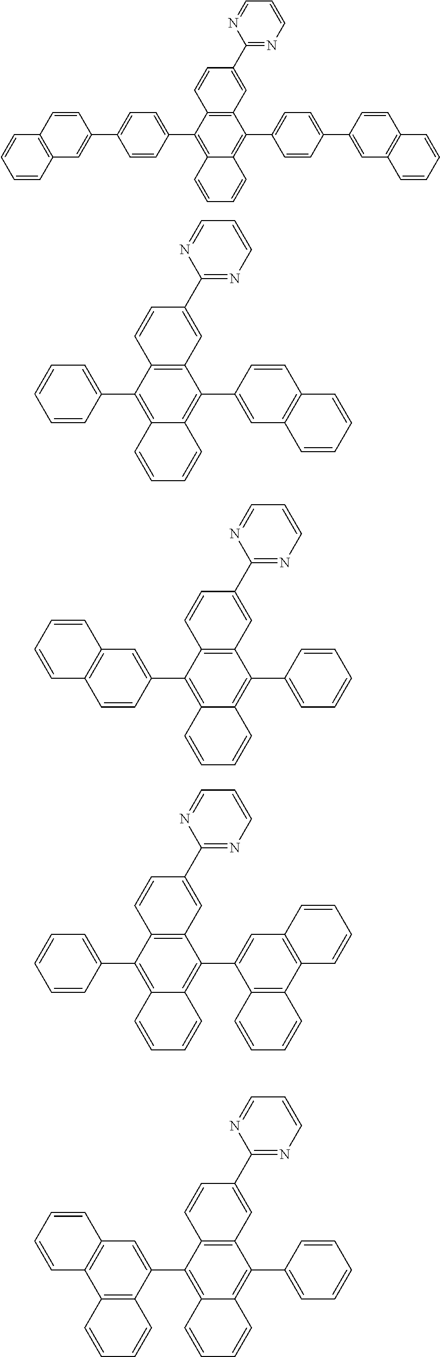

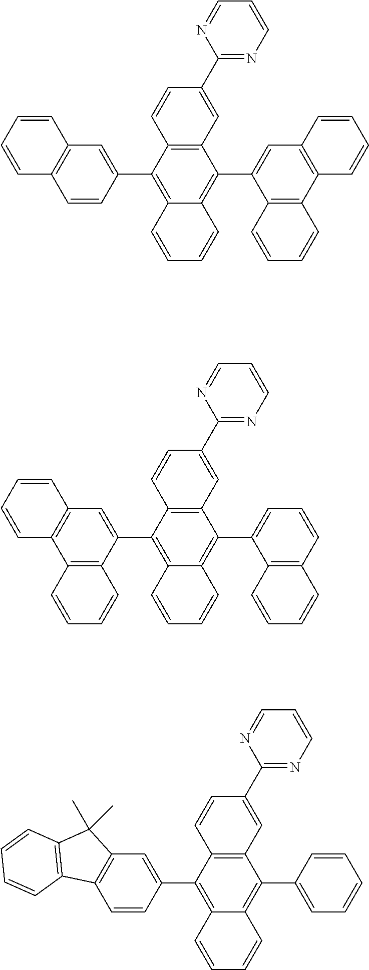 [SMILES: c1cnc(-c2ccc3c(-c4ccc5ccccc5c4)c4ccccc4c(-c4cc5ccccc5c5ccccc45)c3c2)nc1 ; c1cnc(-c2ccc3c(-c4cc5ccccc5c5ccccc45)c4ccccc4c(-c4cccc5ccccc45)c3c2)nc1 ; CC1(C)c2ccccc2-c2ccc(-c3c4ccccc4c(-c4ccccc4)c4cc(-c5ncccn5)ccc34)cc21]

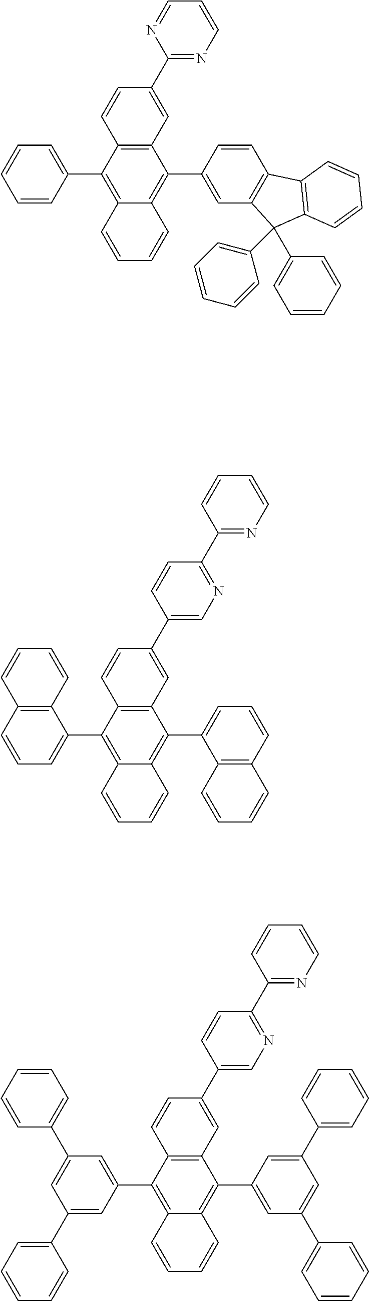 [SMILES: c1ccc(-c2c3ccccc3c(-c3ccc4c(c3)C(c3ccccc3)(c3ccccc3)c3ccccc3-4)c3cc(-c4ncccn4)ccc23)cc1 ; c1ccc(-c2ccc(-c3ccc4c(-c5cccc6ccccc56)c5ccccc5c(-c5cccc6ccccc56)c4c3)cn2)nc1 ; c1ccc(-c2cc(-c3ccccc3)cc(-c3c4ccccc4c(-c4cc(-c5ccccc5)cc(-c5ccccc5)c4)c4cc(-c5ccc(-c6ccccn6)nc5)ccc34)c2)cc1]

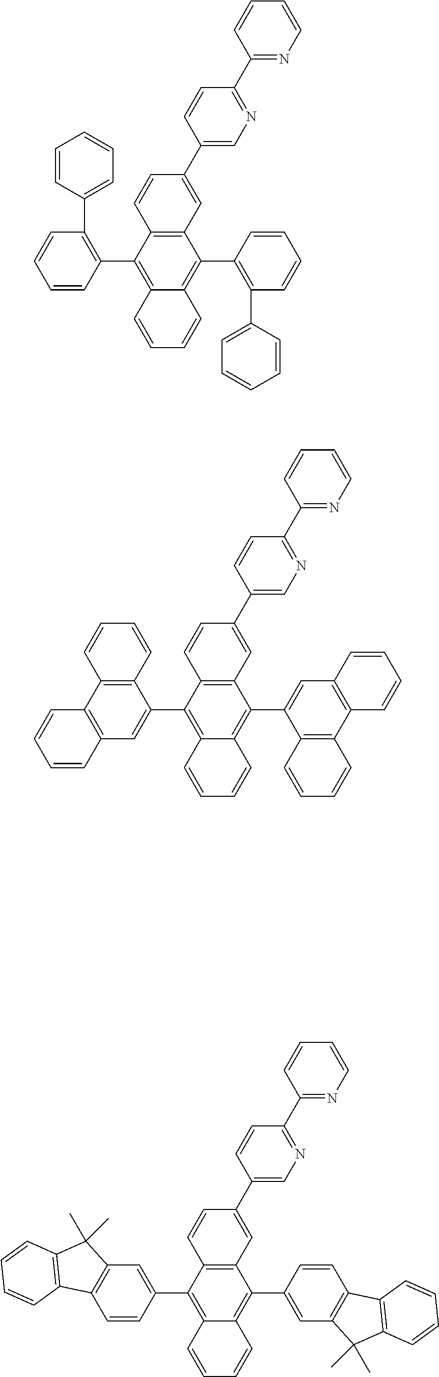 [SMILES: c1ccc(-c2ccccc2-c2c3ccccc3c(-c3ccccc3-c3ccccc3)c3cc(-c4ccc(-c5ccccn5)nc4)ccc23)cc1 ; c1ccc(-c2ccc(-c3ccc4c(-c5cc6ccccc6c6ccccc56)c5ccccc5c(-c5cc6ccccc6c6ccccc56)c4c3)cn2)nc1 ; CC1(C)c2ccccc2-c2ccc(-c3c4ccccc4c(-c4ccc5c(c4)C(C)(C)c4ccccc4-5)c4cc(-c5ccc(-c6ccccn6)nc5)ccc34)cc21]

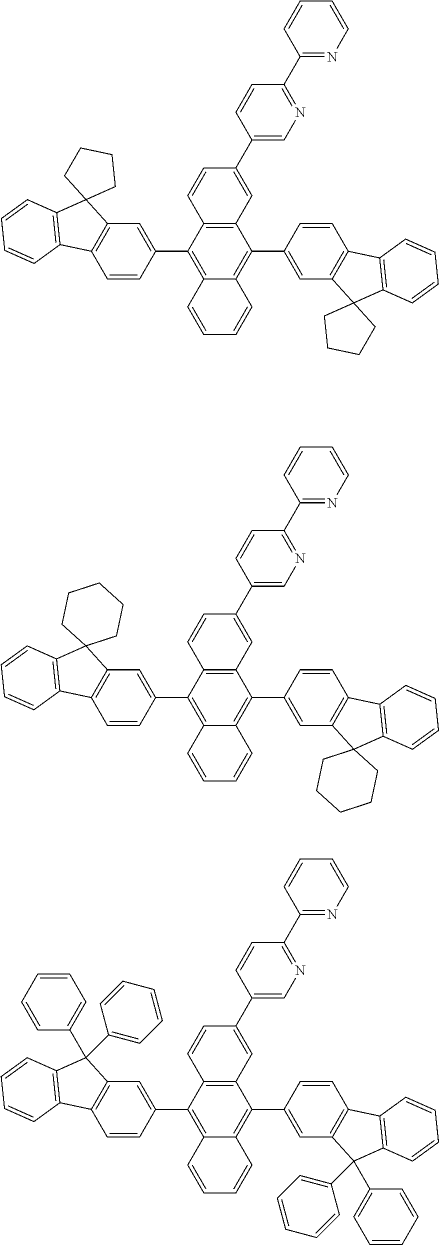 [SMILES: c1ccc(-c2ccc(-c3ccc4c(-c5ccc6c(c5)C5(CCCC5)c5ccccc5-6)c5ccccc5c(-c5ccc6c(c5)C5(CCCC5)c5ccccc5-6)c4c3)cn2)nc1 ; c1ccc(-c2ccc(-c3ccc4c(-c5ccc6c(c5)C5(CCCCC5)c5ccccc5-6)c5ccccc5c(-c5ccc6c(c5)C5(CCCCC5)c5ccccc5-6)c4c3)cn2)nc1 ; c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7ccccn7)nc6)ccc45)cc32)cc1]

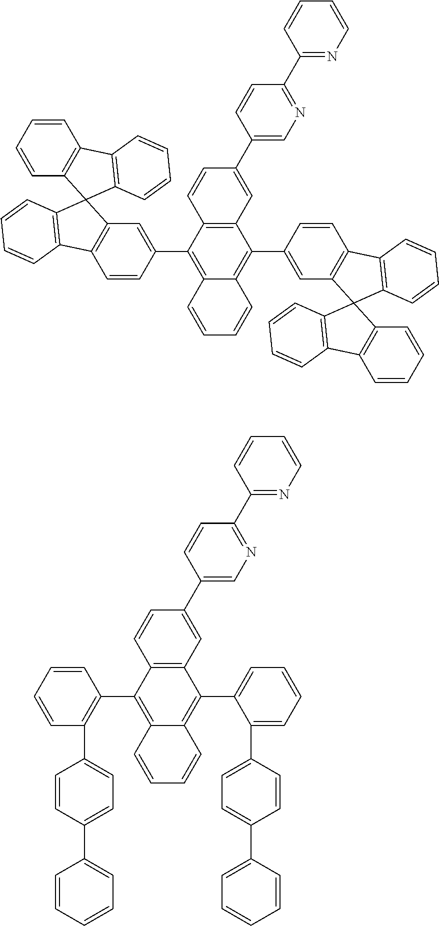 [SMILES: c1ccc(-c2ccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)cn2)nc1 ; c1ccc(-c2ccc(-c3ccccc3-c3c4ccccc4c(-c4ccccc4-c4ccc(-c5ccccc5)cc4)c4cc(-c5ccc(-c6ccccn6)nc5)ccc34)cc2)cc1]

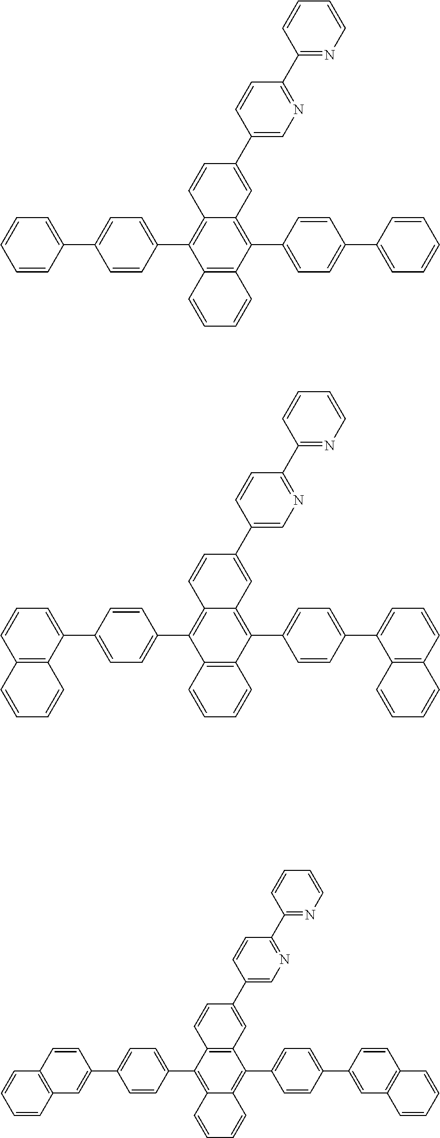 [SMILES: c1ccc(-c2ccc(-c3c4ccccc4c(-c4ccc(-c5ccccc5)cc4)c4cc(-c5ccc(-c6ccccn6)nc5)ccc34)cc2)cc1 ; c1ccc(-c2ccc(-c3ccc4c(-c5ccc(-c6cccc7ccccc67)cc5)c5ccccc5c(-c5ccc(-c6cccc7ccccc67)cc5)c4c3)cn2)nc1 ; c1ccc(-c2ccc(-c3ccc4c(-c5ccc(-c6ccc7ccccc7c6)cc5)c5ccccc5c(-c5ccc(-c6ccc7ccccc7c6)cc5)c4c3)cn2)nc1]

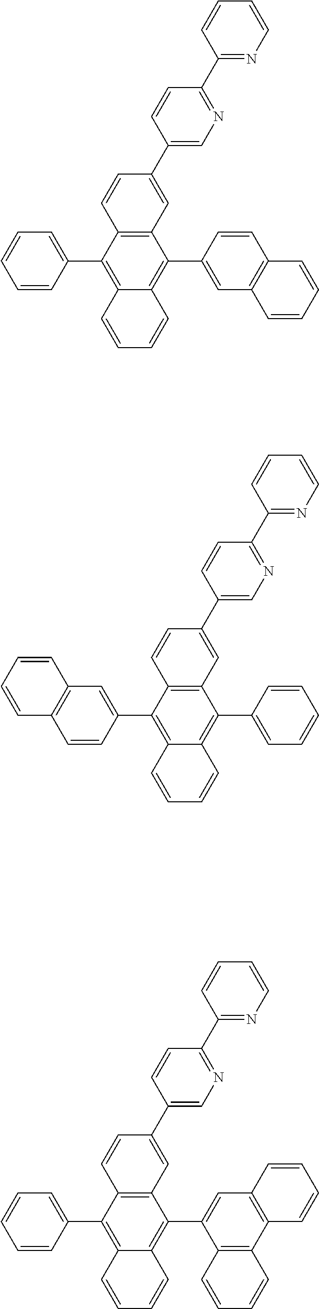 [SMILES: c1ccc(-c2c3ccccc3c(-c3ccc4ccccc4c3)c3cc(-c4ccc(-c5ccccn5)nc4)ccc23)cc1 ; c1ccc(-c2c3ccccc3c(-c3ccc4ccccc4c3)c3ccc(-c4ccc(-c5ccccn5)nc4)cc23)cc1 ; c1ccc(-c2c3ccccc3c(-c3cc4ccccc4c4ccccc34)c3cc(-c4ccc(-c5ccccn5)nc4)ccc23)cc1]

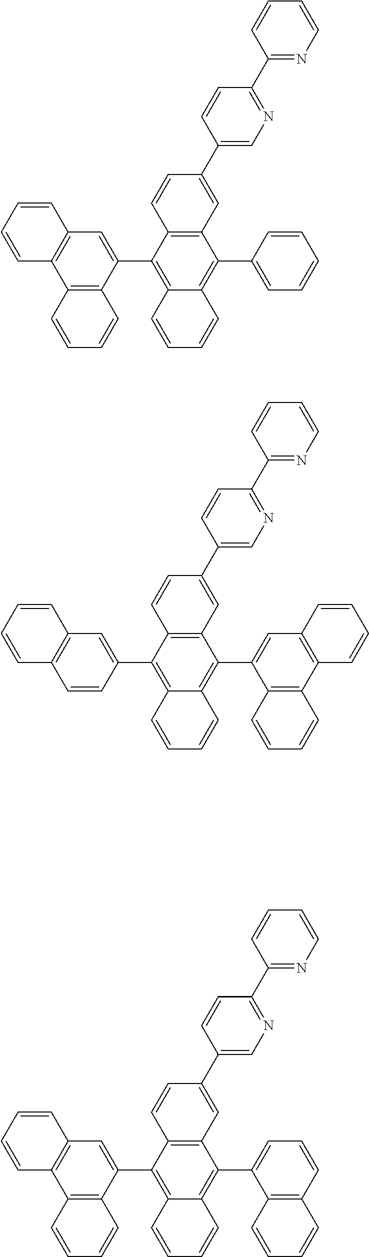 [SMILES: c1ccc(-c2c3ccccc3c(-c3cc4ccccc4c4ccccc34)c3ccc(-c4ccc(-c5ccccn5)nc4)cc23)cc1 ; c1ccc(-c2ccc(-c3ccc4c(-c5ccc6ccccc6c5)c5ccccc5c(-c5cc6ccccc6c6ccccc56)c4c3)cn2)nc1 ; c1ccc(-c2ccc(-c3ccc4c(-c5cc6ccccc6c6ccccc56)c5ccccc5c(-c5cccc6ccccc56)c4c3)cn2)nc1]

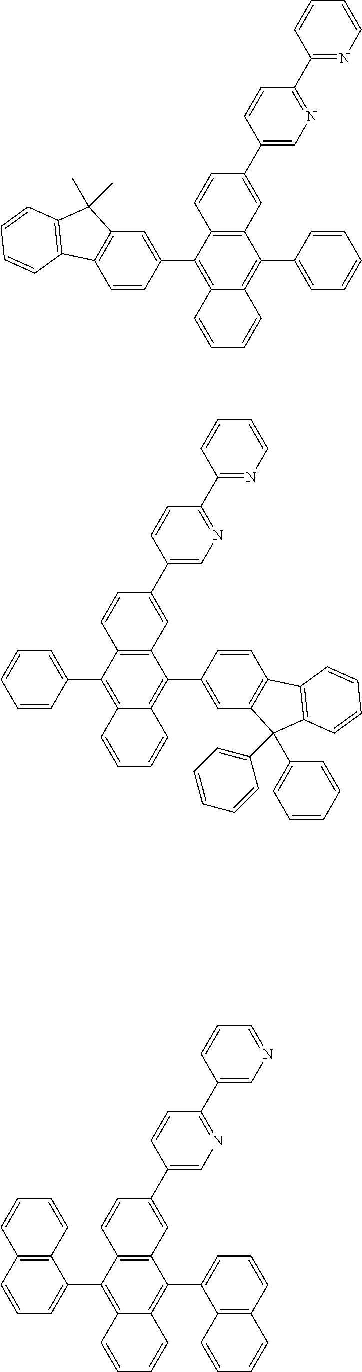 [SMILES: CC1(C)c2ccccc2-c2ccc(-c3c4ccccc4c(-c4ccccc4)c4cc(-c5ccc(-c6ccccn6)nc5)ccc34)cc21 ; c1ccc(-c2c3ccccc3c(-c3ccc4c(c3)C(c3ccccc3)(c3ccccc3)c3ccccc3-4)c3cc(-c4ccc(-c5ccccn5)nc4)ccc23)cc1 ; c1cncc(-c2ccc(-c3ccc4c(-c5cccc6ccccc56)c5ccccc5c(-c5cccc6ccccc56)c4c3)cn2)c1]

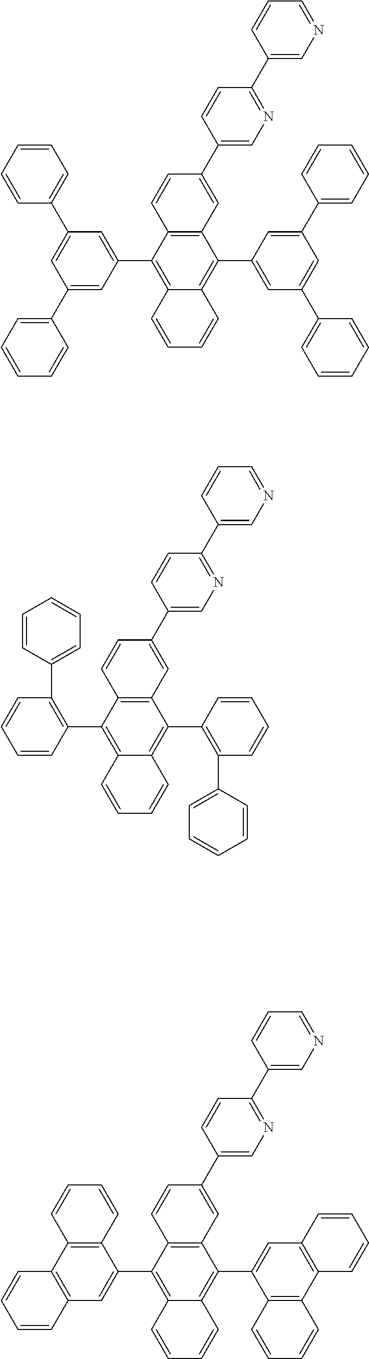 [SMILES: c1ccc(-c2cc(-c3ccccc3)cc(-c3c4ccccc4c(-c4cc(-c5ccccc5)cc(-c5ccccc5)c4)c4cc(-c5ccc(-c6cccnc6)nc5)ccc34)c2)cc1 ; c1ccc(-c2ccccc2-c2c3ccccc3c(-c3ccccc3-c3ccccc3)c3cc(-c4ccc(-c5cccnc5)nc4)ccc23)cc1 ; c1cncc(-c2ccc(-c3ccc4c(-c5cc6ccccc6c6ccccc56)c5ccccc5c(-c5cc6ccccc6c6ccccc56)c4c3)cn2)c1]

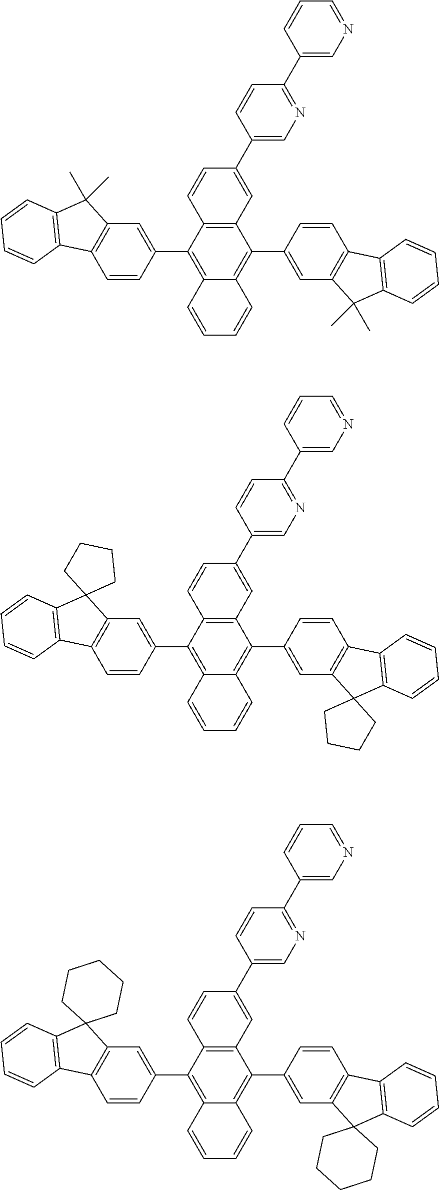 [SMILES: CC1(C)c2ccccc2-c2ccc(-c3c4ccccc4c(-c4ccc5c(c4)C(C)(C)c4ccccc4-5)c4cc(-c5ccc(-c6cccnc6)nc5)ccc34)cc21 ; c1cncc(-c2ccc(-c3ccc4c(-c5ccc6c(c5)C5(CCCC5)c5ccccc5-6)c5ccccc5c(-c5ccc6c(c5)C5(CCCC5)c5ccccc5-6)c4c3)cn2)c1 ; c1cncc(-c2ccc(-c3ccc4c(-c5ccc6c(c5)C5(CCCCC5)c5ccccc5-6)c5ccccc5c(-c5ccc6c(c5)C5(CCCCC5)c5ccccc5-6)c4c3)cn2)c1]

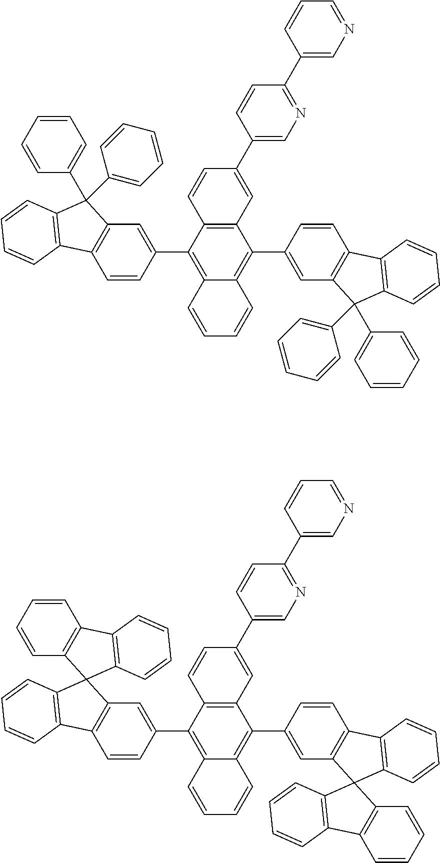 [SMILES: c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7cccnc7)nc6)ccc45)cc32)cc1 ; c1cncc(-c2ccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)cn2)c1]

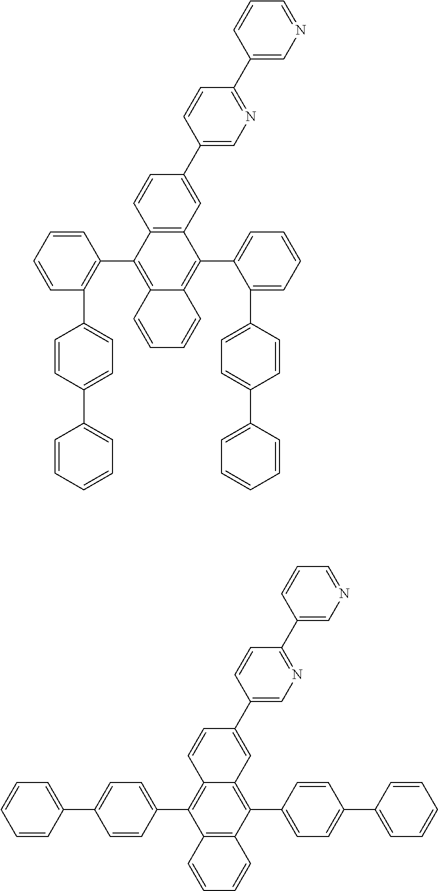 [SMILES: c1ccc(-c2ccc(-c3ccccc3-c3c4ccccc4c(-c4ccccc4-c4ccc(-c5ccccc5)cc4)c4cc(-c5ccc(-c6cccnc6)nc5)ccc34)cc2)cc1 ; c1ccc(-c2ccc(-c3c4ccccc4c(-c4ccc(-c5ccccc5)cc4)c4cc(-c5ccc(-c6cccnc6)nc5)ccc34)cc2)cc1]

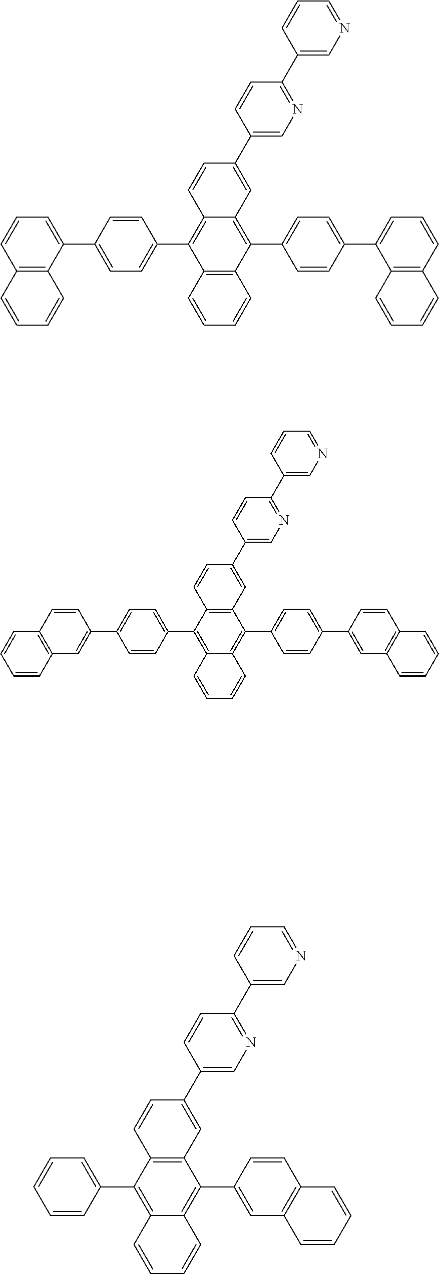 [SMILES: c1cncc(-c2ccc(-c3ccc4c(-c5ccc(-c6cccc7ccccc67)cc5)c5ccccc5c(-c5ccc(-c6cccc7ccccc67)cc5)c4c3)cn2)c1 ; c1cncc(-c2ccc(-c3ccc4c(-c5ccc(-c6ccc7ccccc7c6)cc5)c5ccccc5c(-c5ccc(-c6ccc7ccccc7c6)cc5)c4c3)cn2)c1 ; c1ccc(-c2c3ccccc3c(-c3ccc4ccccc4c3)c3cc(-c4ccc(-c5cccnc5)nc4)ccc23)cc1]

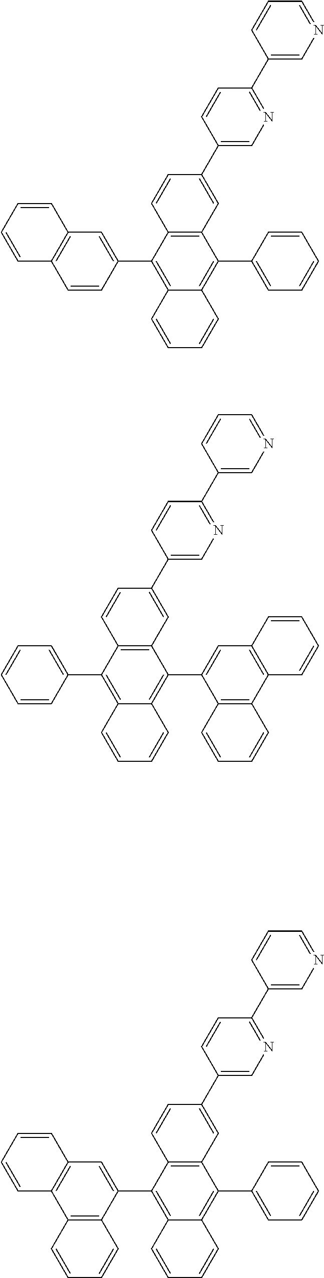 [SMILES: c1ccc(-c2c3ccccc3c(-c3ccc4ccccc4c3)c3ccc(-c4ccc(-c5cccnc5)nc4)cc23)cc1 ; c1ccc(-c2c3ccccc3c(-c3cc4ccccc4c4ccccc34)c3cc(-c4ccc(-c5cccnc5)nc4)ccc23)cc1 ; c1ccc(-c2c3ccccc3c(-c3cc4ccccc4c4ccccc34)c3ccc(-c4ccc(-c5cccnc5)nc4)cc23)cc1]

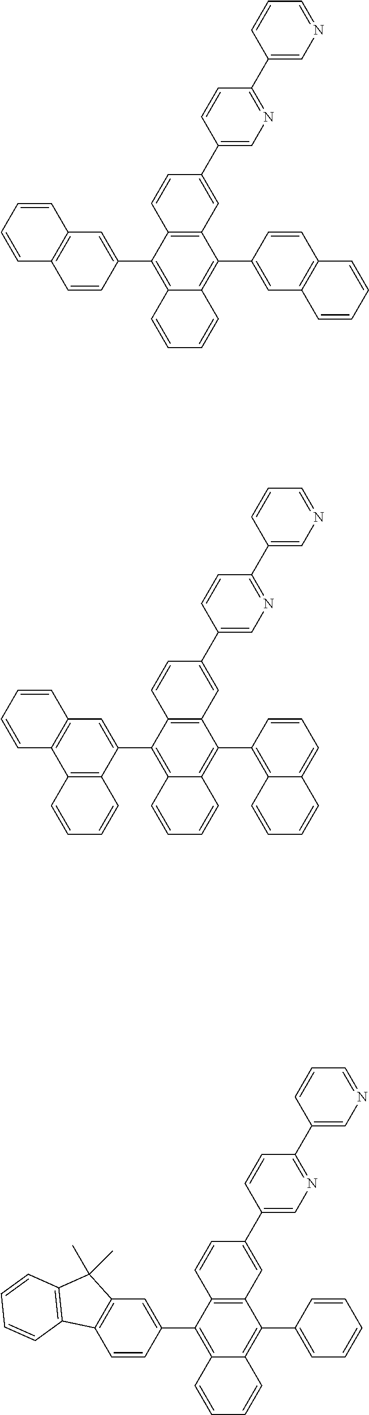 [SMILES: c1cncc(-c2ccc(-c3ccc4c(-c5ccc6ccccc6c5)c5ccccc5c(-c5ccc6ccccc6c5)c4c3)cn2)c1 ; c1cncc(-c2ccc(-c3ccc4c(-c5cc6ccccc6c6ccccc56)c5ccccc5c(-c5cccc6ccccc56)c4c3)cn2)c1 ; CC1(C)c2ccccc2-c2ccc(-c3c4ccccc4c(-c4ccccc4)c4cc(-c5ccc(-c6cccnc6)nc5)ccc34)cc21]

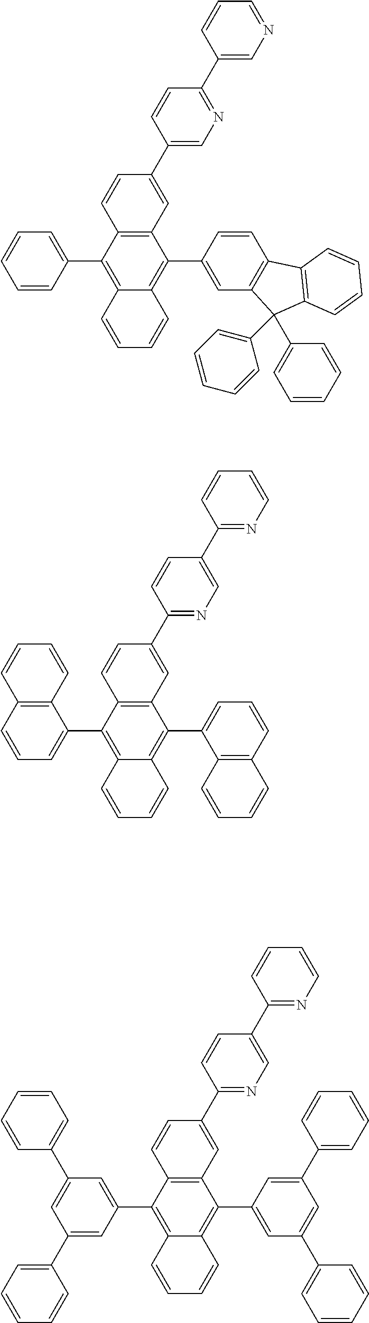 [SMILES: c1ccc(-c2c3ccccc3c(-c3ccc4c(c3)C(c3ccccc3)(c3ccccc3)c3ccccc3-4)c3cc(-c4ccc(-c5cccnc5)nc4)ccc23)cc1 ; c1ccc(-c2ccc(-c3ccc4c(-c5cccc6ccccc56)c5ccccc5c(-c5cccc6ccccc56)c4c3)nc2)nc1 ; c1ccc(-c2cc(-c3ccccc3)cc(-c3c4ccccc4c(-c4cc(-c5ccccc5)cc(-c5ccccc5)c4)c4cc(-c5ccc(-c6ccccn6)cn5)ccc34)c2)cc1]

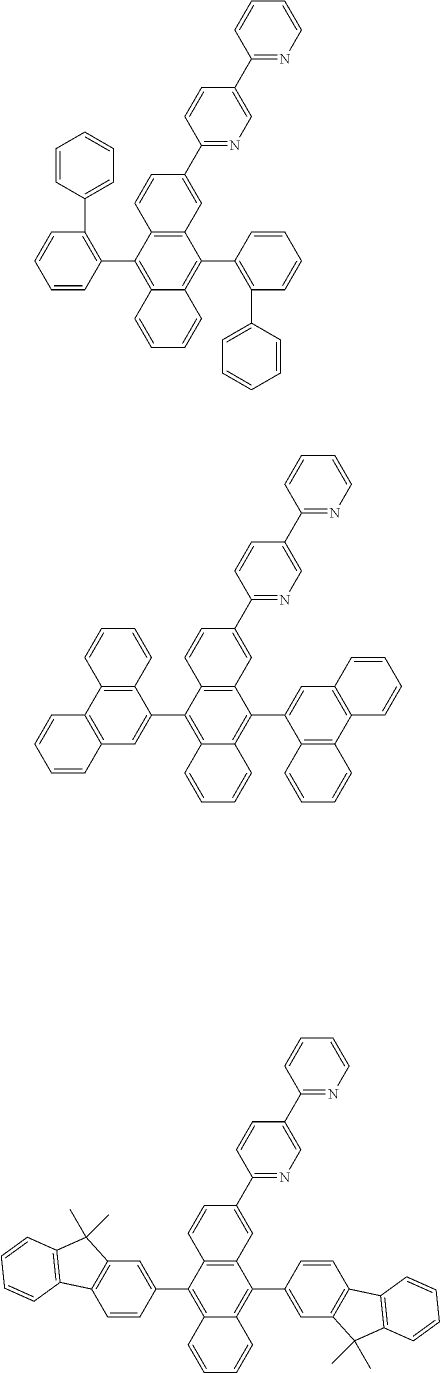 [SMILES: c1ccc(-c2ccccc2-c2c3ccccc3c(-c3ccccc3-c3ccccc3)c3cc(-c4ccc(-c5ccccn5)cn4)ccc23)cc1 ; c1ccc(-c2ccc(-c3ccc4c(-c5cc6ccccc6c6ccccc56)c5ccccc5c(-c5cc6ccccc6c6ccccc56)c4c3)nc2)nc1 ; CC1(C)c2ccccc2-c2ccc(-c3c4ccccc4c(-c4ccc5c(c4)C(C)(C)c4ccccc4-5)c4cc(-c5ccc(-c6ccccn6)cn5)ccc34)cc21]

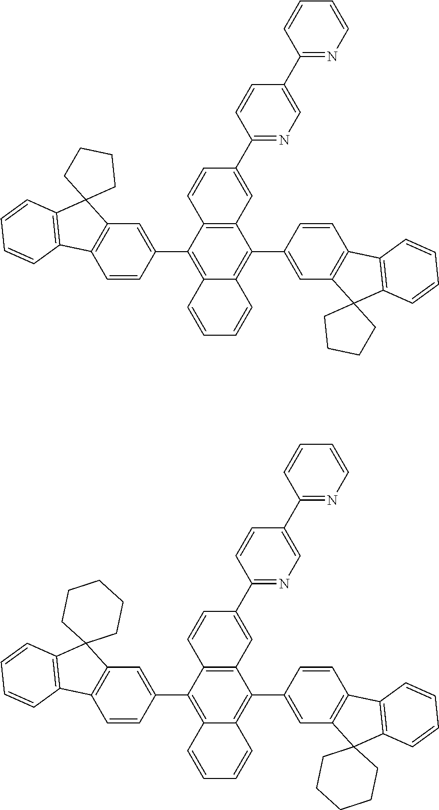 [SMILES: c1ccc(-c2ccc(-c3ccc4c(-c5ccc6c(c5)C5(CCCC5)c5ccccc5-6)c5ccccc5c(-c5ccc6c(c5)C5(CCCC5)c5ccccc5-6)c4c3)nc2)nc1 ; c1ccc(-c2ccc(-c3ccc4c(-c5ccc6c(c5)C5(CCCCC5)c5ccccc5-6)c5ccccc5c(-c5ccc6c(c5)C5(CCCCC5)c5ccccc5-6)c4c3)nc2)nc1]

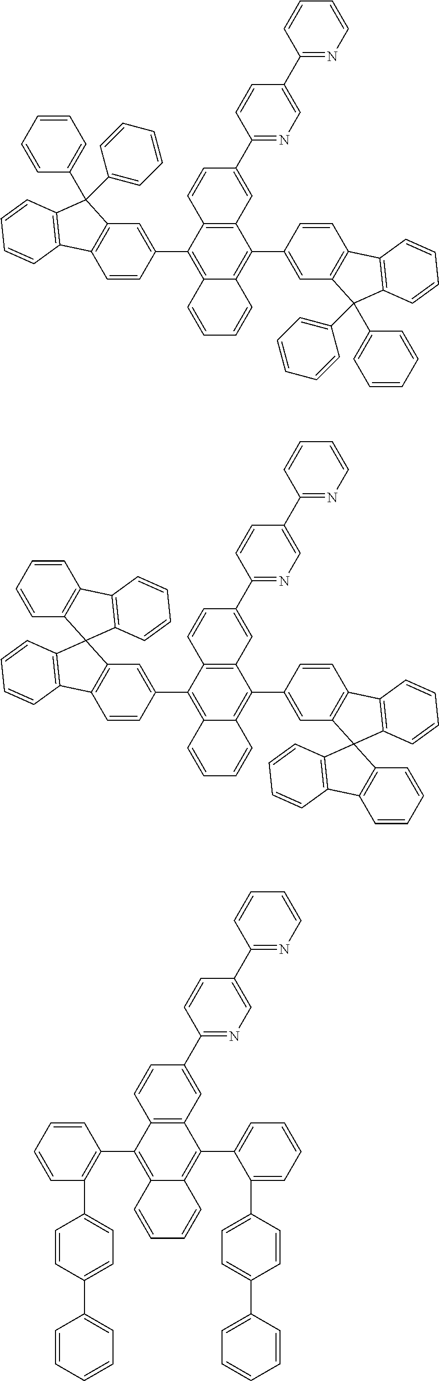 [SMILES: c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7ccccn7)cn6)ccc45)cc32)cc1 ; c1ccc(-c2ccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)nc2)nc1 ; c1ccc(-c2ccc(-c3ccccc3-c3c4ccccc4c(-c4ccccc4-c4ccc(-c5ccccc5)cc4)c4cc(-c5ccc(-c6ccccn6)cn5)ccc34)cc2)cc1]

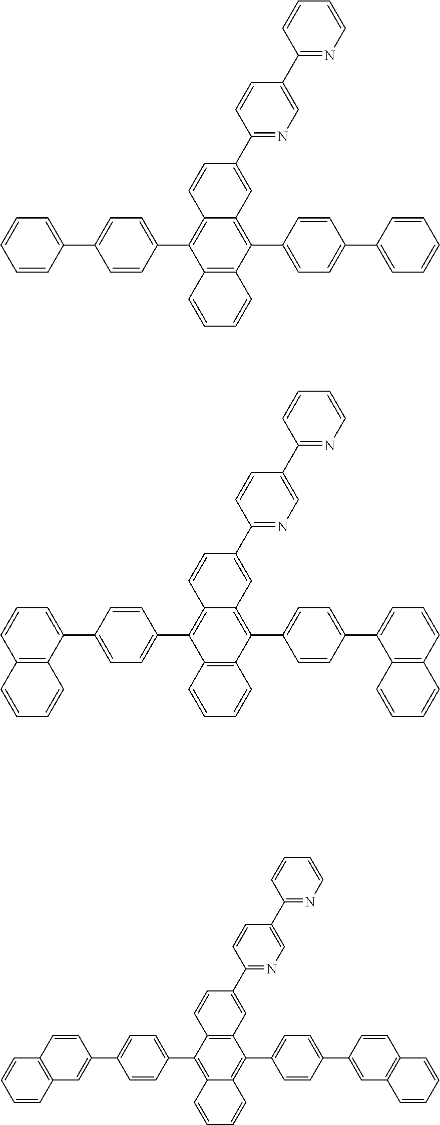 [SMILES: c1ccc(-c2ccc(-c3c4ccccc4c(-c4ccc(-c5ccccc5)cc4)c4cc(-c5ccc(-c6ccccn6)cn5)ccc34)cc2)cc1 ; c1ccc(-c2ccc(-c3ccc4c(-c5ccc(-c6cccc7ccccc67)cc5)c5ccccc5c(-c5ccc(-c6cccc7ccccc67)cc5)c4c3)nc2)nc1 ; c1ccc(-c2ccc(-c3ccc4c(-c5ccc(-c6ccc7ccccc7c6)cc5)c5ccccc5c(-c5ccc(-c6ccc7ccccc7c6)cc5)c4c3)nc2)nc1]

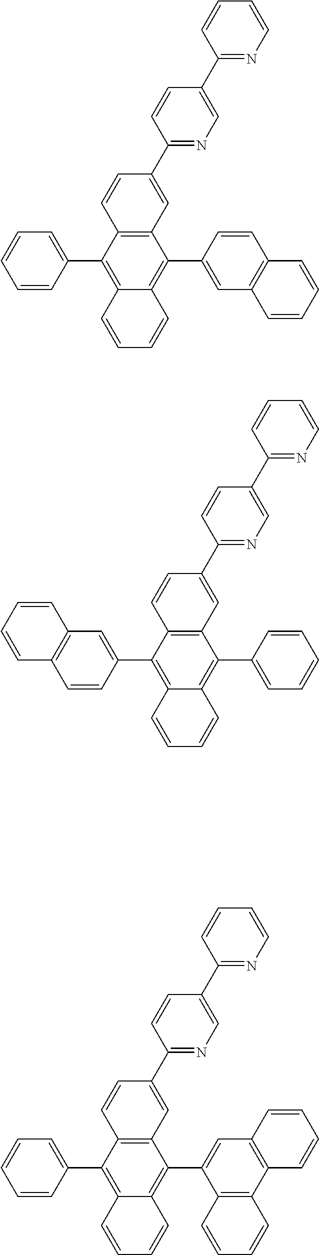 [SMILES: c1ccc(-c2c3ccccc3c(-c3ccc4ccccc4c3)c3cc(-c4ccc(-c5ccccn5)cn4)ccc23)cc1 ; c1ccc(-c2c3ccccc3c(-c3ccc4ccccc4c3)c3ccc(-c4ccc(-c5ccccn5)cn4)cc23)cc1 ; c1ccc(-c2c3ccccc3c(-c3cc4ccccc4c4ccccc34)c3cc(-c4ccc(-c5ccccn5)cn4)ccc23)cc1]

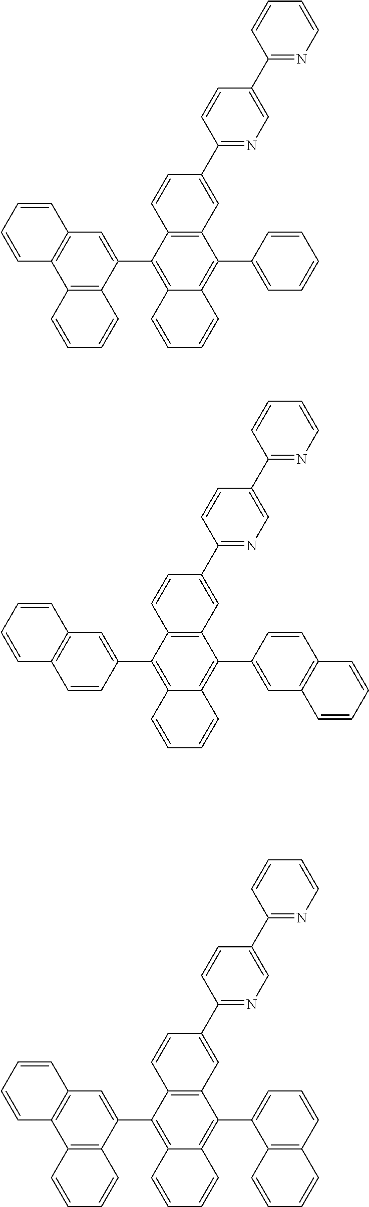 [SMILES: c1ccc(-c2c3ccccc3c(-c3cc4ccccc4c4ccccc34)c3ccc(-c4ccc(-c5ccccn5)cn4)cc23)cc1 ; c1ccc(-c2ccc(-c3ccc4c(-c5ccc6ccccc6c5)c5ccccc5c(-c5ccc6ccccc6c5)c4c3)nc2)nc1 ; c1ccc(-c2ccc(-c3ccc4c(-c5cc6ccccc6c6ccccc56)c5ccccc5c(-c5cccc6ccccc56)c4c3)nc2)nc1]

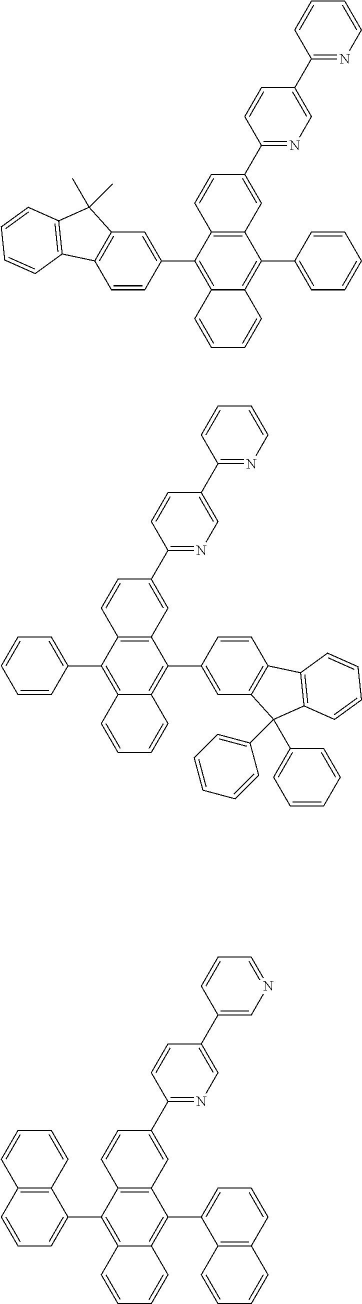 [SMILES: CC1(C)c2ccccc2-c2ccc(-c3c4ccccc4c(-c4ccccc4)c4cc(-c5ccc(-c6ccccn6)cn5)ccc34)cc21 ; c1ccc(-c2c3ccccc3c(-c3ccc4c(c3)C(c3ccccc3)(c3ccccc3)c3ccccc3-4)c3cc(-c4ccc(-c5ccccn5)cn4)ccc23)cc1 ; c1cncc(-c2ccc(-c3ccc4c(-c5cccc6ccccc56)c5ccccc5c(-c5cccc6ccccc56)c4c3)nc2)c1]

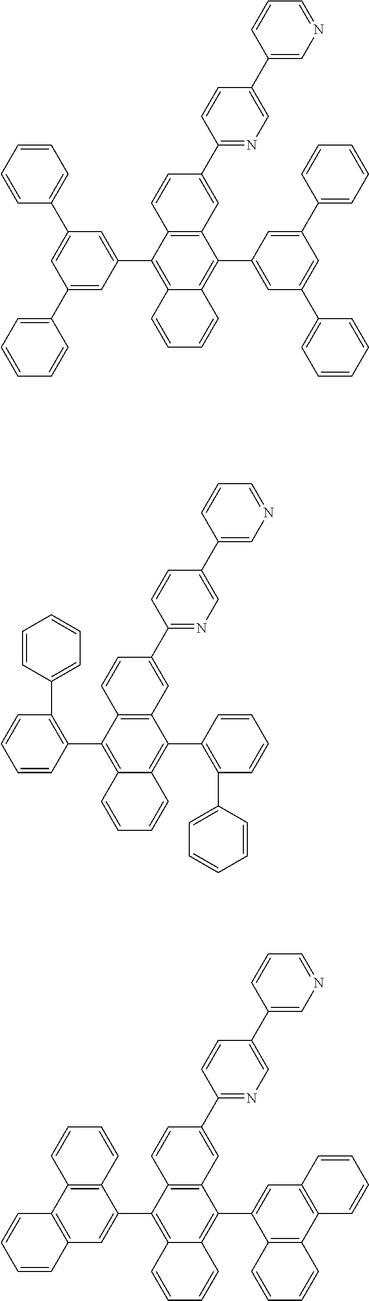 [SMILES: c1ccc(-c2cc(-c3ccccc3)cc(-c3c4ccccc4c(-c4cc(-c5ccccc5)cc(-c5ccccc5)c4)c4cc(-c5ccc(-c6cccnc6)cn5)ccc34)c2)cc1 ; c1ccc(-c2ccccc2-c2c3ccccc3c(-c3ccccc3-c3ccccc3)c3cc(-c4ccc(-c5cccnc5)cn4)ccc23)cc1 ; c1cncc(-c2ccc(-c3ccc4c(-c5cc6ccccc6c6ccccc56)c5ccccc5c(-c5cc6ccccc6c6ccccc56)c4c3)nc2)c1]

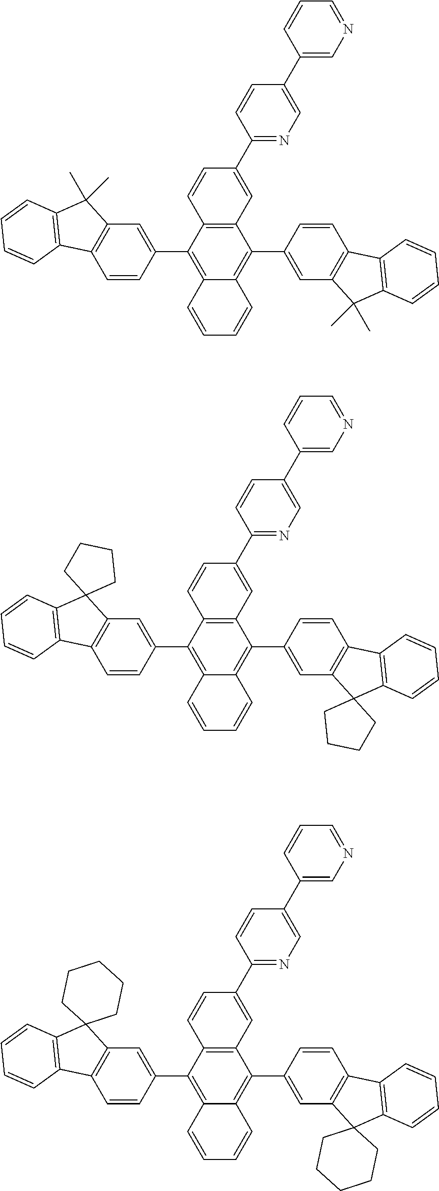 [SMILES: CC1(C)c2ccccc2-c2ccc(-c3c4ccccc4c(-c4ccc5c(c4)C(C)(C)c4ccccc4-5)c4cc(-c5ccc(-c6cccnc6)cn5)ccc34)cc21 ; c1cncc(-c2ccc(-c3ccc4c(-c5ccc6c(c5)C5(CCCC5)c5ccccc5-6)c5ccccc5c(-c5ccc6c(c5)C5(CCCC5)c5ccccc5-6)c4c3)nc2)c1 ; c1cncc(-c2ccc(-c3ccc4c(-c5ccc6c(c5)C5(CCCCC5)c5ccccc5-6)c5ccccc5c(-c5ccc6c(c5)C5(CCCCC5)c5ccccc5-6)c4c3)nc2)c1]

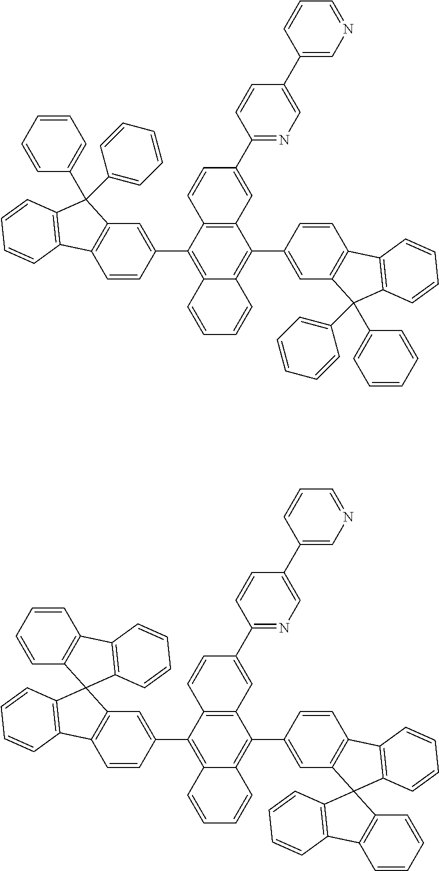 [SMILES: c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7cccnc7)cn6)ccc45)cc32)cc1 ; c1cncc(-c2ccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)nc2)c1]

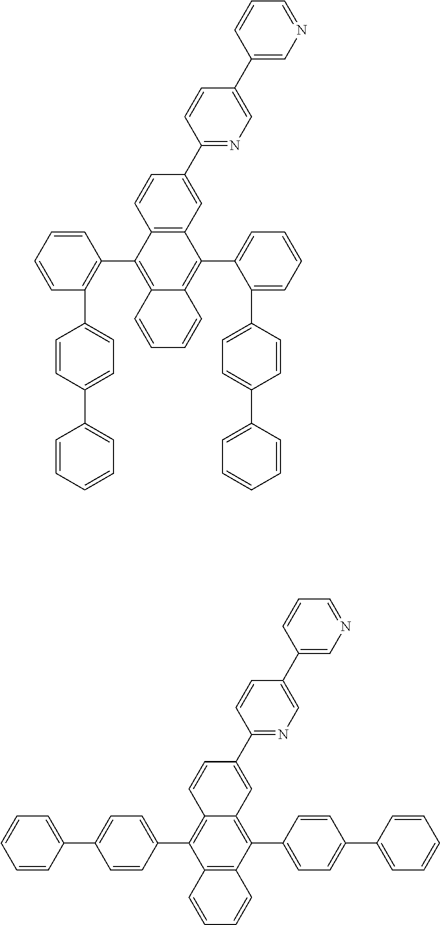 [SMILES: c1ccc(-c2ccc(-c3ccccc3-c3c4ccccc4c(-c4ccccc4-c4ccc(-c5ccccc5)cc4)c4cc(-c5ccc(-c6cccnc6)cn5)ccc34)cc2)cc1 ; c1ccc(-c2ccc(-c3c4ccccc4c(-c4ccc(-c5ccccc5)cc4)c4cc(-c5ccc(-c6cccnc6)cn5)ccc34)cc2)cc1]

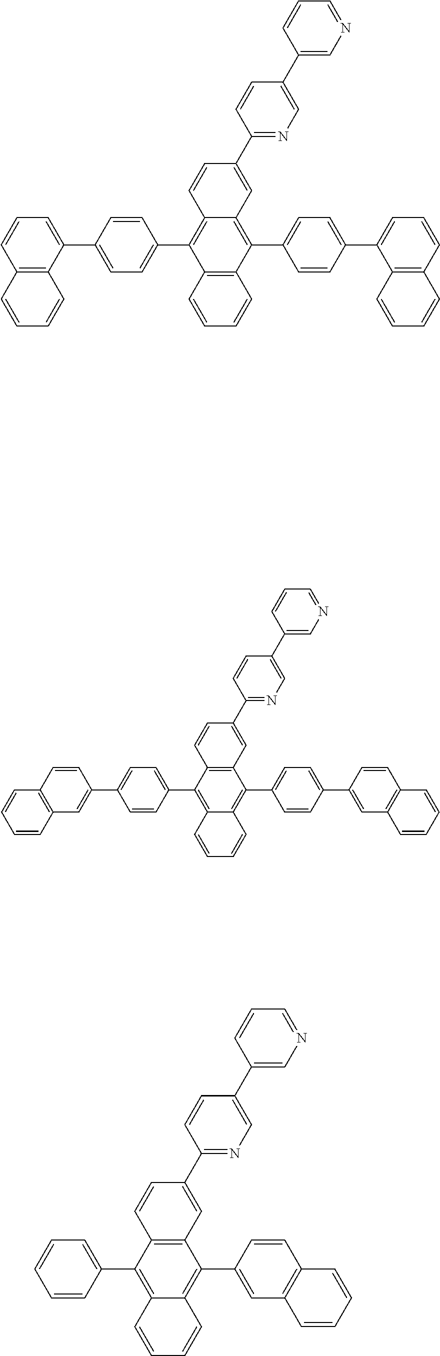 [SMILES: c1cncc(-c2ccc(-c3ccc4c(-c5ccc(-c6cccc7ccccc67)cc5)c5ccccc5c(-c5ccc(-c6cccc7ccccc67)cc5)c4c3)nc2)c1 ; c1cncc(-c2ccc(-c3ccc4c(-c5ccc(-c6ccc7ccccc7c6)cc5)c5ccccc5c(-c5ccc(-c6ccc7ccccc7c6)cc5)c4c3)nc2)c1 ; c1ccc(-c2c3ccccc3c(-c3ccc4ccccc4c3)c3cc(-c4ccc(-c5cccnc5)cn4)ccc23)cc1]

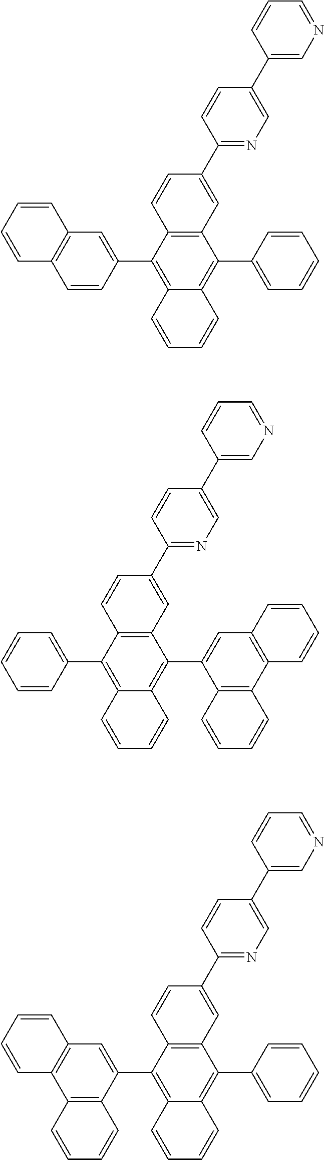 [SMILES: c1ccc(-c2c3ccccc3c(-c3ccc4ccccc4c3)c3ccc(-c4ccc(-c5cccnc5)cn4)cc23)cc1 ; c1ccc(-c2c3ccccc3c(-c3cc4ccccc4c4ccccc34)c3cc(-c4ccc(-c5cccnc5)cn4)ccc23)cc1 ; c1ccc(-c2c3ccccc3c(-c3cc4ccccc4c4ccccc34)c3ccc(-c4ccc(-c5cccnc5)cn4)cc23)cc1]

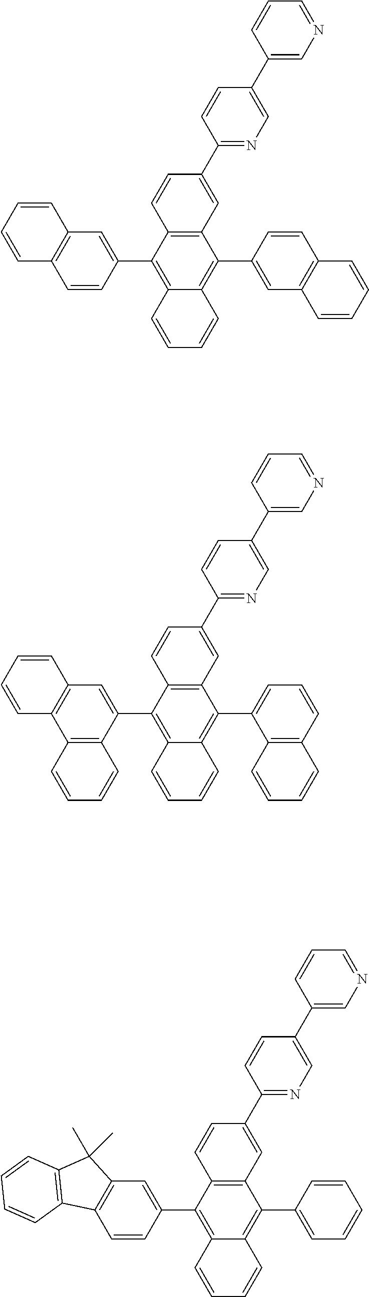 [SMILES: c1cncc(-c2ccc(-c3ccc4c(-c5ccc6ccccc6c5)c5ccccc5c(-c5ccc6ccccc6c5)c4c3)nc2)c1 ; c1cncc(-c2ccc(-c3ccc4c(-c5cc6ccccc6c6ccccc56)c5ccccc5c(-c5cccc6ccccc56)c4c3)nc2)c1 ; CC1(C)c2ccccc2-c2ccc(-c3c4ccccc4c(-c4ccccc4)c4cc(-c5ccc(-c6cccnc6)cn5)ccc34)cc21]

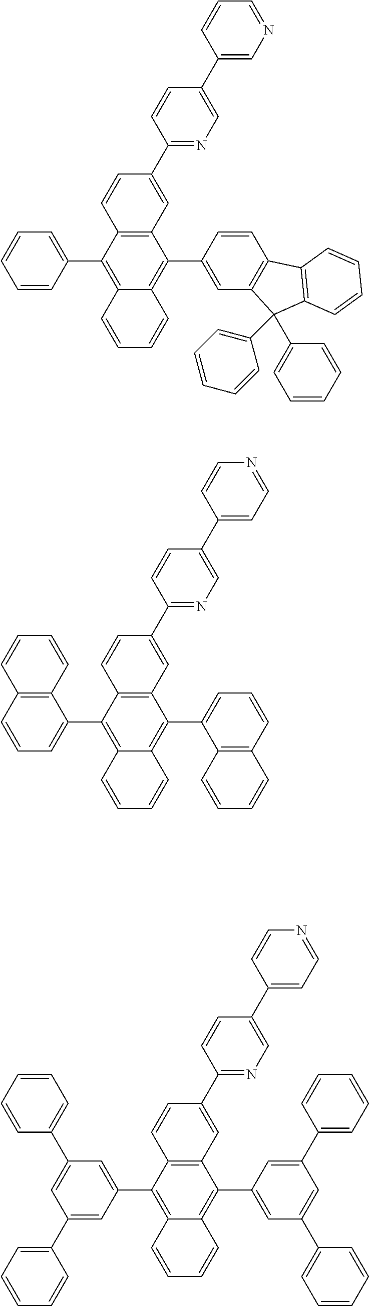 [SMILES: c1ccc(-c2c3ccccc3c(-c3ccc4c(c3)C(c3ccccc3)(c3ccccc3)c3ccccc3-4)c3cc(-c4ccc(-c5cccnc5)cn4)ccc23)cc1 ; c1ccc2c(-c3c4ccccc4c(-c4cccc5ccccc45)c4cc(-c5ccc(-c6ccncc6)cn5)ccc34)cccc2c1 ; c1ccc(-c2cc(-c3ccccc3)cc(-c3c4ccccc4c(-c4cc(-c5ccccc5)cc(-c5ccccc5)c4)c4cc(-c5ccc(-c6ccncc6)cn5)ccc34)c2)cc1]

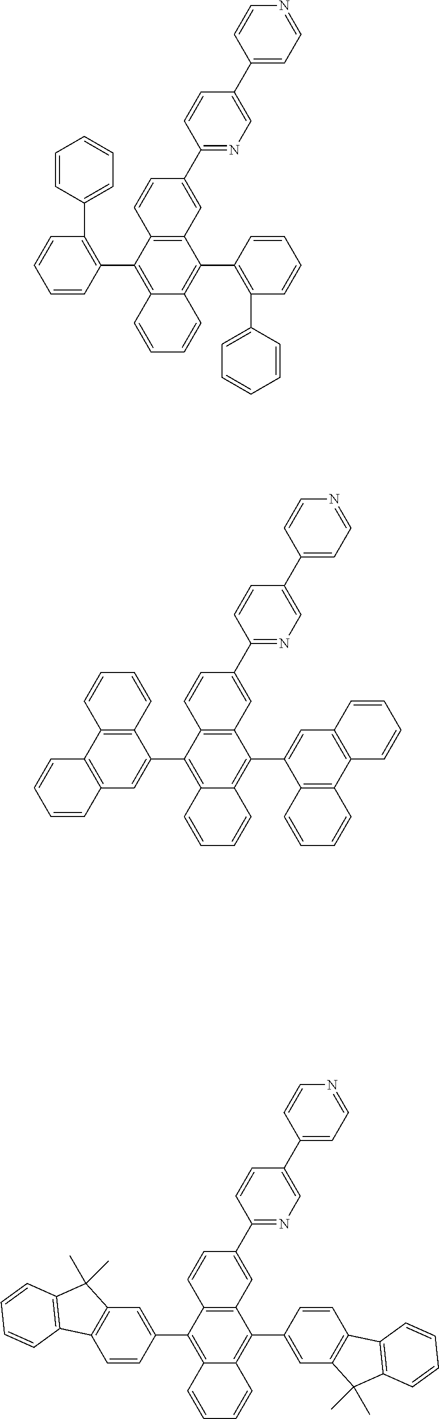 [SMILES: c1ccc(-c2ccccc2-c2c3ccccc3c(-c3ccccc3-c3ccccc3)c3cc(-c4ccc(-c5ccncc5)cn4)ccc23)cc1 ; c1ccc2c(c1)cc(-c1c3ccccc3c(-c3cc4ccccc4c4ccccc34)c3cc(-c4ccc(-c5ccncc5)cn4)ccc13)c1ccccc12 ; CC1(C)c2ccccc2-c2ccc(-c3c4ccccc4c(-c4ccc5c(c4)C(C)(C)c4ccccc4-5)c4cc(-c5ccc(-c6ccncc6)cn5)ccc34)cc21]

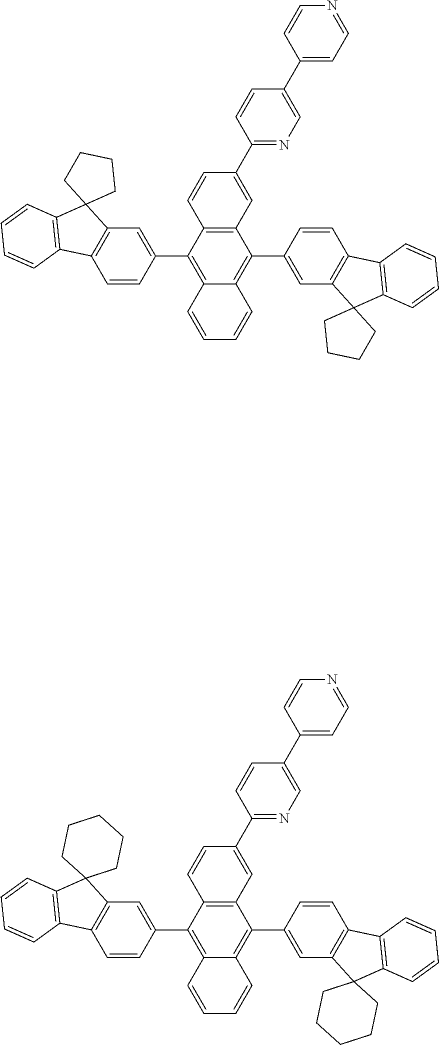 [SMILES: c1ccc2c(c1)-c1ccc(-c3c4ccccc4c(-c4ccc5c(c4)C4(CCCC4)c4ccccc4-5)c4cc(-c5ccc(-c6ccncc6)cn5)ccc34)cc1C21CCCC1 ; c1ccc2c(c1)-c1ccc(-c3c4ccccc4c(-c4ccc5c(c4)C4(CCCCC4)c4ccccc4-5)c4cc(-c5ccc(-c6ccncc6)cn5)ccc34)cc1C21CCCCC1]

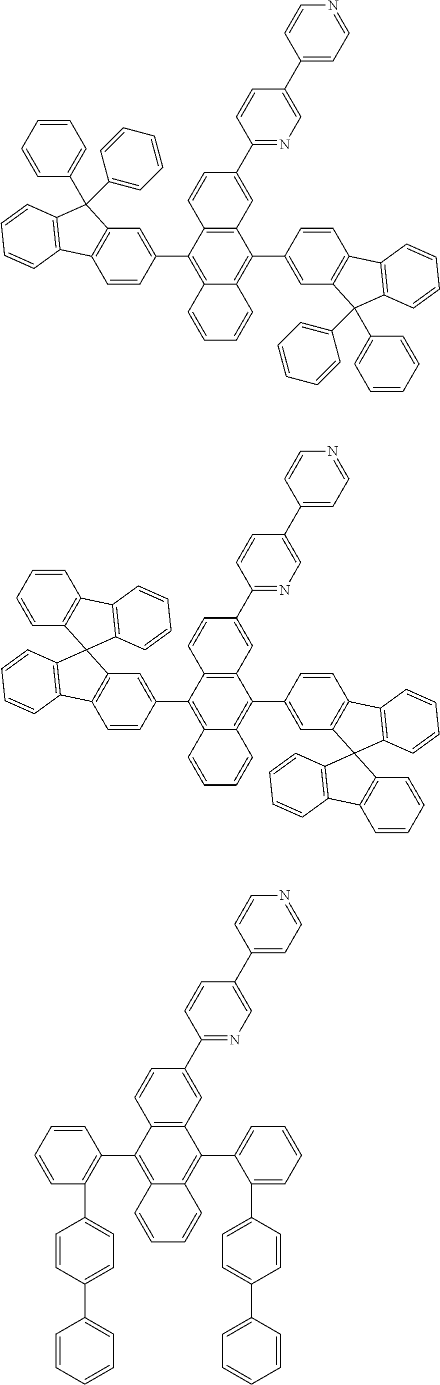 [SMILES: c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7ccncc7)cn6)ccc45)cc32)cc1 ; c1ccc2c(c1)-c1ccccc1C21c2ccccc2-c2ccc(-c3c4ccccc4c(-c4ccc5c(c4)C4(c6ccccc6-c6ccccc64)c4ccccc4-5)c4cc(-c5ccc(-c6ccncc6)cn5)ccc34)cc21 ; c1ccc(-c2ccc(-c3ccccc3-c3c4ccccc4c(-c4ccccc4-c4ccc(-c5ccccc5)cc4)c4cc(-c5ccc(-c6ccncc6)cn5)ccc34)cc2)cc1]

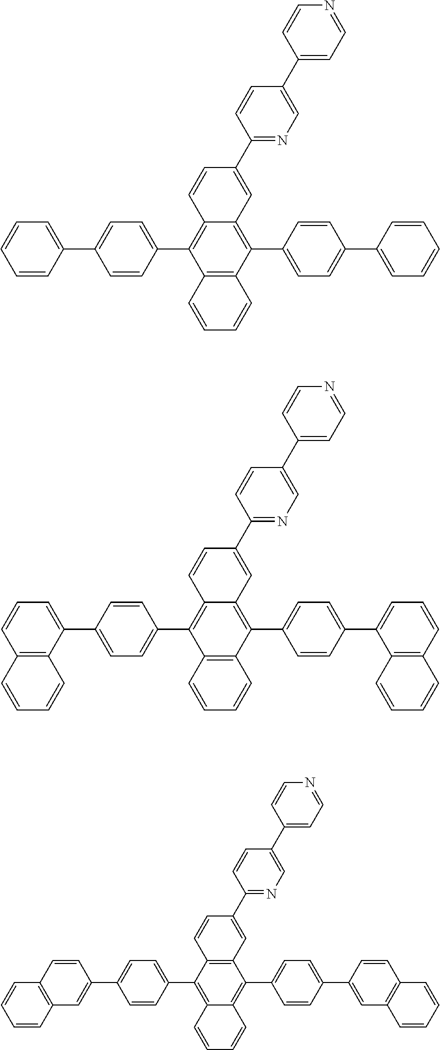 [SMILES: c1ccc(-c2ccc(-c3c4ccccc4c(-c4ccc(-c5ccccc5)cc4)c4cc(-c5ccc(-c6ccncc6)cn5)ccc34)cc2)cc1 ; c1ccc2c(-c3ccc(-c4c5ccccc5c(-c5ccc(-c6cccc7ccccc67)cc5)c5cc(-c6ccc(-c7ccncc7)cn6)ccc45)cc3)cccc2c1 ; c1ccc2cc(-c3ccc(-c4c5ccccc5c(-c5ccc(-c6ccc7ccccc7c6)cc5)c5cc(-c6ccc(-c7ccncc7)cn6)ccc45)cc3)ccc2c1]

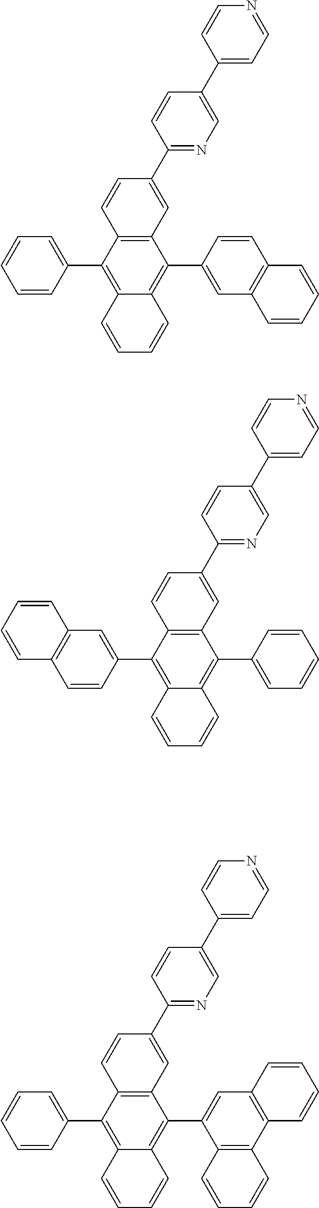 [SMILES: c1ccc(-c2c3ccccc3c(-c3ccc4ccccc4c3)c3cc(-c4ccc(-c5ccncc5)cn4)ccc23)cc1 ; c1ccc(-c2c3ccccc3c(-c3ccc4ccccc4c3)c3ccc(-c4ccc(-c5ccncc5)cn4)cc23)cc1 ; c1ccc(-c2c3ccccc3c(-c3cc4ccccc4c4ccccc34)c3cc(-c4ccc(-c5ccncc5)cn4)ccc23)cc1]

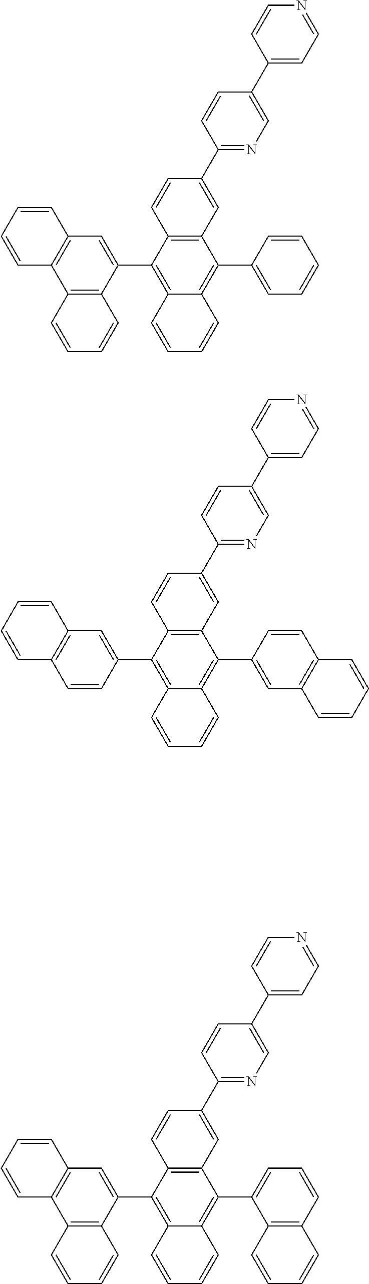 [SMILES: c1ccc(-c2c3ccccc3c(-c3cc4ccccc4c4ccccc34)c3ccc(-c4ccc(-c5ccncc5)cn4)cc23)cc1 ; c1ccc2cc(-c3c4ccccc4c(-c4ccc5ccccc5c4)c4cc(-c5ccc(-c6ccncc6)cn5)ccc34)ccc2c1 ; c1ccc2c(-c3c4ccccc4c(-c4cc5ccccc5c5ccccc45)c4ccc(-c5ccc(-c6ccncc6)cn5)cc34)cccc2c1]

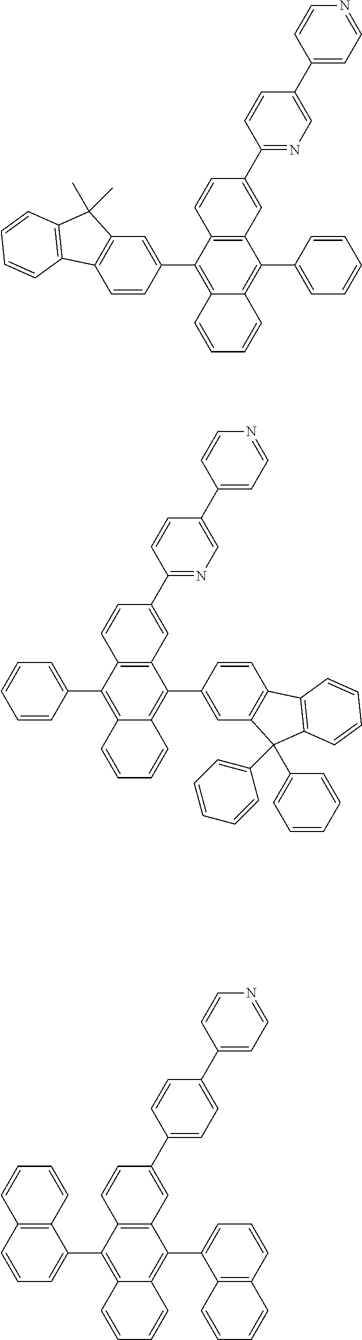 [SMILES: CC1(C)c2ccccc2-c2ccc(-c3c4ccccc4c(-c4ccccc4)c4cc(-c5ccc(-c6ccncc6)cn5)ccc34)cc21 ; c1ccc(-c2c3ccccc3c(-c3ccc4c(c3)C(c3ccccc3)(c3ccccc3)c3ccccc3-4)c3cc(-c4ccc(-c5ccncc5)cn4)ccc23)cc1 ; c1ccc2c(-c3c4ccccc4c(-c4cccc5ccccc45)c4cc(-c5ccc(-c6ccncc6)cc5)ccc34)cccc2c1]

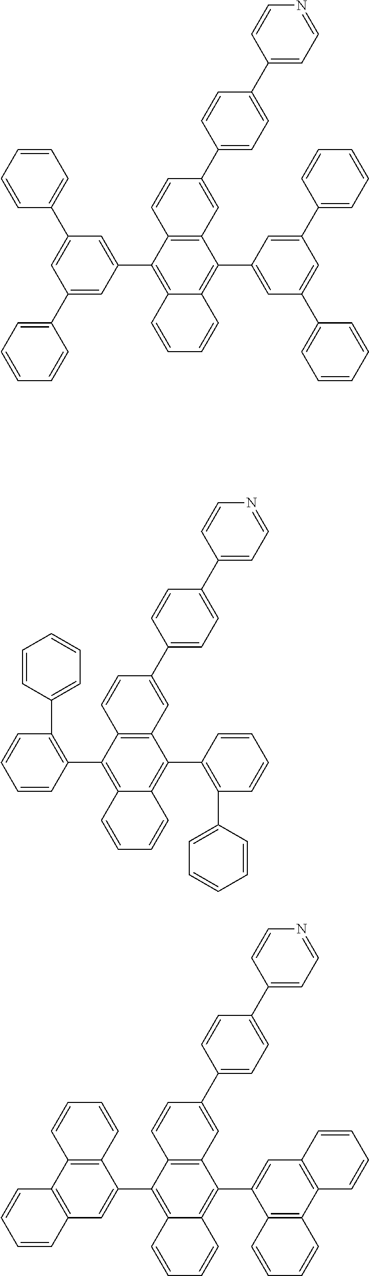 [SMILES: c1ccc(-c2cc(-c3ccccc3)cc(-c3c4ccccc4c(-c4cc(-c5ccccc5)cc(-c5ccccc5)c4)c4cc(-c5ccc(-c6ccncc6)cc5)ccc34)c2)cc1 ; c1ccc(-c2ccccc2-c2c3ccccc3c(-c3ccccc3-c3ccccc3)c3cc(-c4ccc(-c5ccncc5)cc4)ccc23)cc1 ; c1ccc2c(c1)cc(-c1c3ccccc3c(-c3cc4ccccc4c4ccccc34)c3cc(-c4ccc(-c5ccncc5)cc4)ccc13)c1ccccc12]

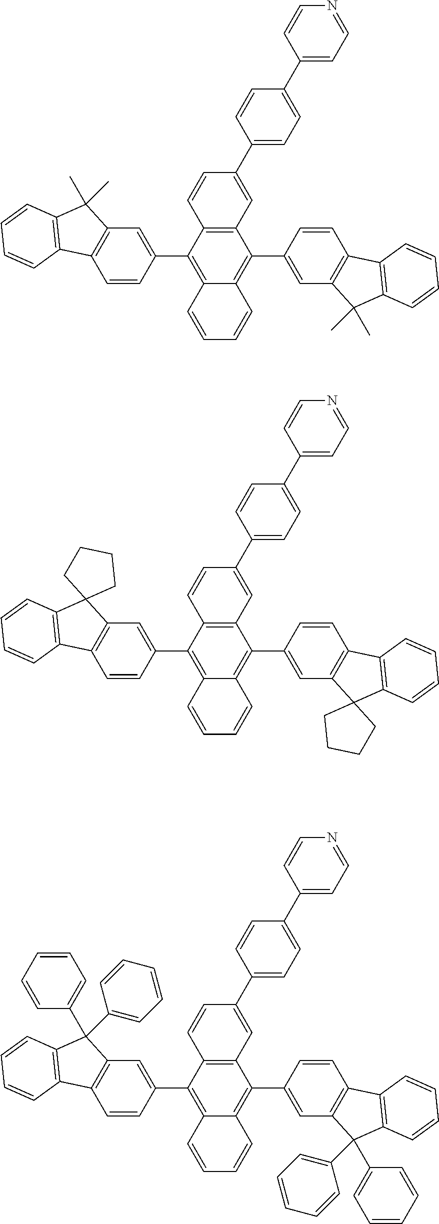 [SMILES: CC1(C)c2ccccc2-c2ccc(-c3c4ccccc4c(-c4ccc5c(c4)C(C)(C)c4ccccc4-5)c4cc(-c5ccc(-c6ccncc6)cc5)ccc34)cc21 ; c1ccc2c(c1)-c1ccc(-c3c4ccccc4c(-c4ccc5c(c4)C4(CCCC4)c4ccccc4-5)c4cc(-c5ccc(-c6ccncc6)cc5)ccc34)cc1C21CCCC1 ; c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7ccncc7)cc6)ccc45)cc32)cc1]

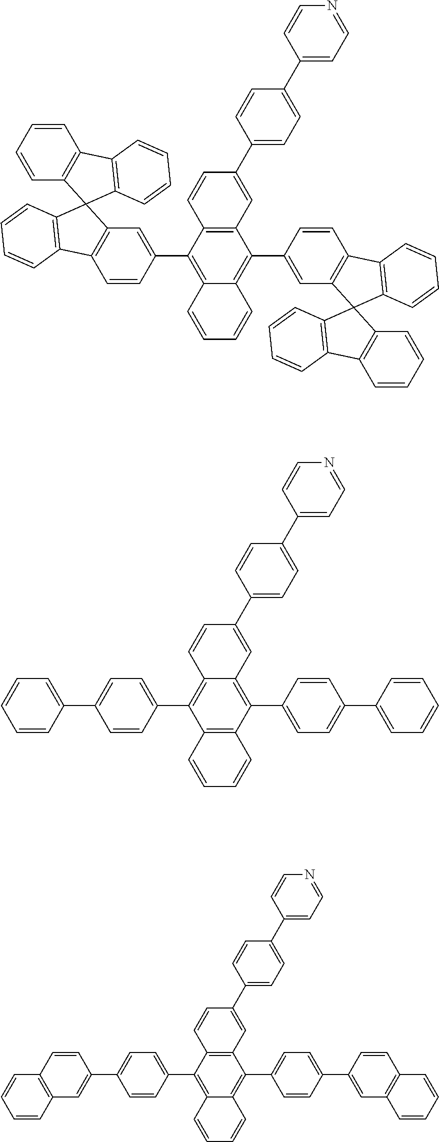 [SMILES: c1ccc2c(c1)-c1ccccc1C21c2ccccc2-c2ccc(-c3c4ccccc4c(-c4ccc5c(c4)C4(c6ccccc6-c6ccccc64)c4ccccc4-5)c4cc(-c5ccc(-c6ccncc6)cc5)ccc34)cc21 ; c1ccc(-c2ccc(-c3c4ccccc4c(-c4ccc(-c5ccccc5)cc4)c4cc(-c5ccc(-c6ccncc6)cc5)ccc34)cc2)cc1 ; c1ccc2cc(-c3ccc(-c4c5ccccc5c(-c5ccc(-c6ccc7ccccc7c6)cc5)c5cc(-c6ccc(-c7ccncc7)cc6)ccc45)cc3)ccc2c1]

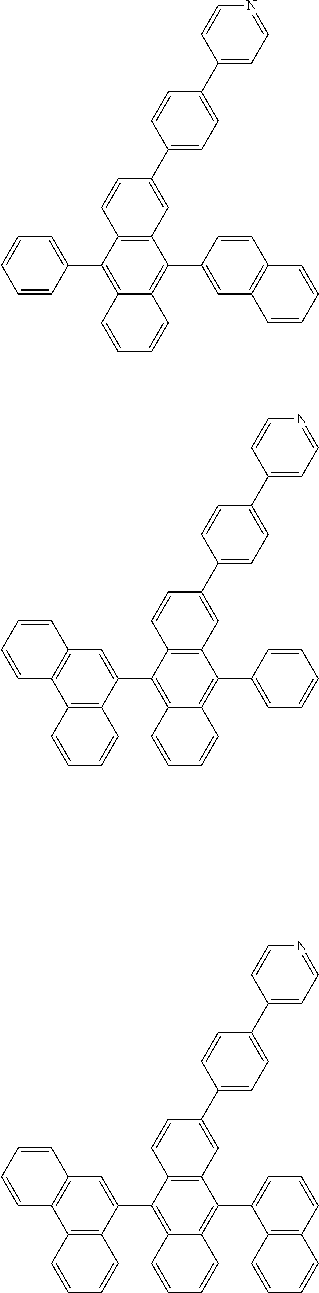 [SMILES: c1ccc(-c2c3ccccc3c(-c3ccc4ccccc4c3)c3cc(-c4ccc(-c5ccncc5)cc4)ccc23)cc1 ; c1ccc(-c2c3ccccc3c(-c3cc4ccccc4c4ccccc34)c3ccc(-c4ccc(-c5ccncc5)cc4)cc23)cc1 ; c1ccc2c(-c3c4ccccc4c(-c4cc5ccccc5c5ccccc45)c4ccc(-c5ccc(-c6ccncc6)cc5)cc34)cccc2c1]

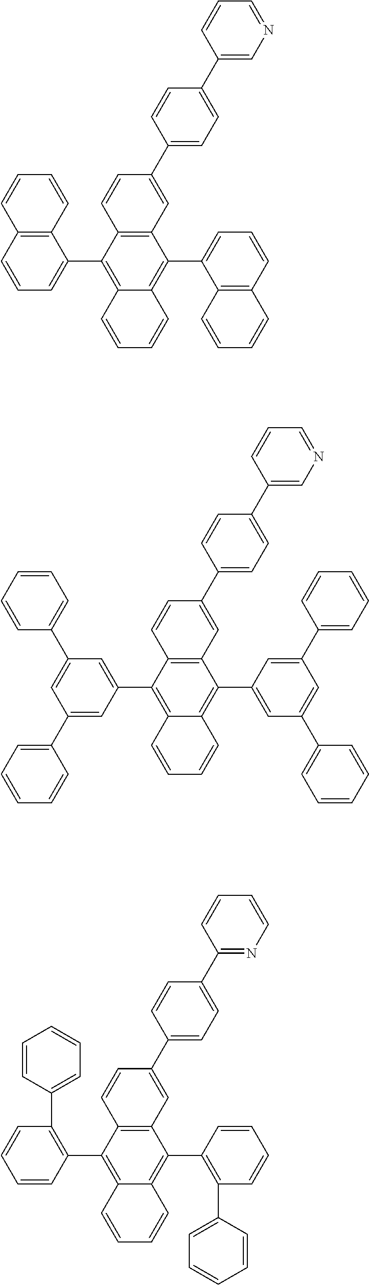 [SMILES: c1cncc(-c2ccc(-c3ccc4c(-c5cccc6ccccc56)c5ccccc5c(-c5cccc6ccccc56)c4c3)cc2)c1 ; c1ccc(-c2cc(-c3ccccc3)cc(-c3c4ccccc4c(-c4cc(-c5ccccc5)cc(-c5ccccc5)c4)c4cc(-c5ccc(-c6cccnc6)cc5)ccc34)c2)cc1 ; c1ccc(-c2ccccc2-c2c3ccccc3c(-c3ccccc3-c3ccccc3)c3cc(-c4ccc(-c5ccccn5)cc4)ccc23)cc1]

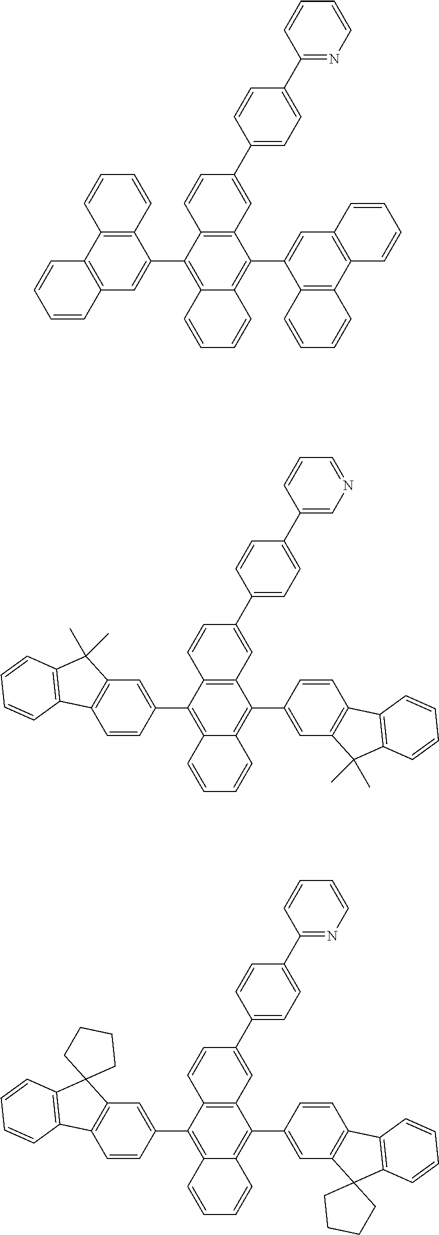 [SMILES: c1ccc(-c2ccc(-c3ccc4c(-c5cc6ccccc6c6ccccc56)c5ccccc5c(-c5cc6ccccc6c6ccccc56)c4c3)cc2)nc1 ; CC1(C)c2ccccc2-c2ccc(-c3c4ccccc4c(-c4ccc5c(c4)C(C)(C)c4ccccc4-5)c4cc(-c5ccc(-c6cccnc6)cc5)ccc34)cc21 ; c1ccc(-c2ccc(-c3ccc4c(-c5ccc6c(c5)C5(CCCC5)c5ccccc5-6)c5ccccc5c(-c5ccc6c(c5)C5(CCCC5)c5ccccc5-6)c4c3)cc2)nc1]

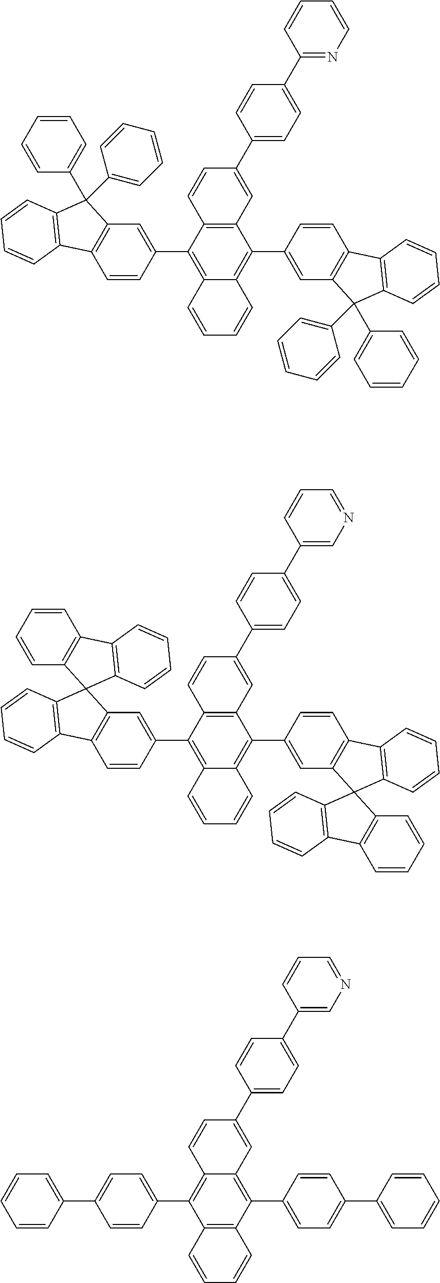 [SMILES: c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7ccccn7)cc6)ccc45)cc32)cc1 ; c1cncc(-c2ccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)cc2)c1 ; c1ccc(-c2ccc(-c3c4ccccc4c(-c4ccc(-c5ccccc5)cc4)c4cc(-c5ccc(-c6cccnc6)cc5)ccc34)cc2)cc1]

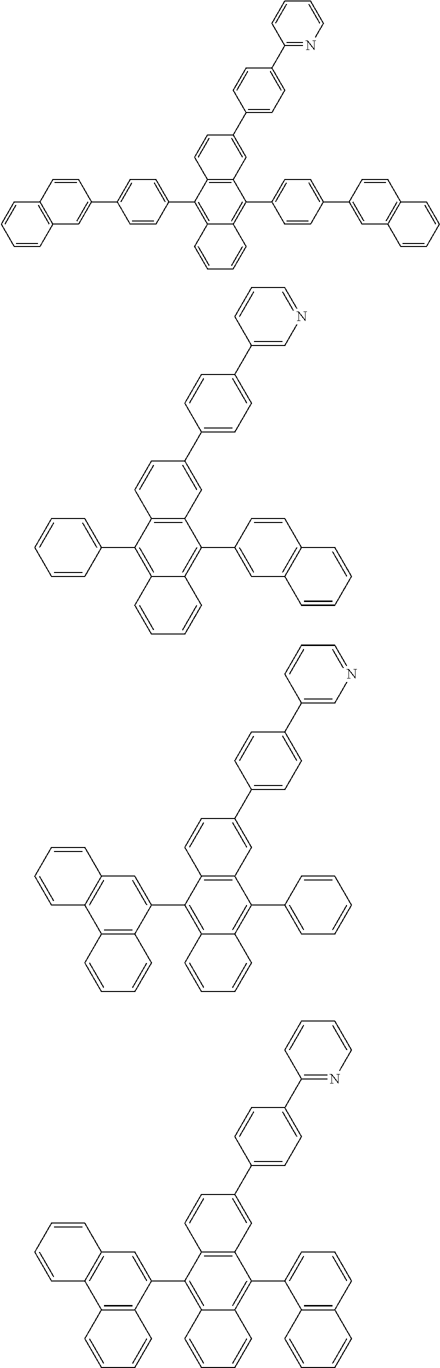

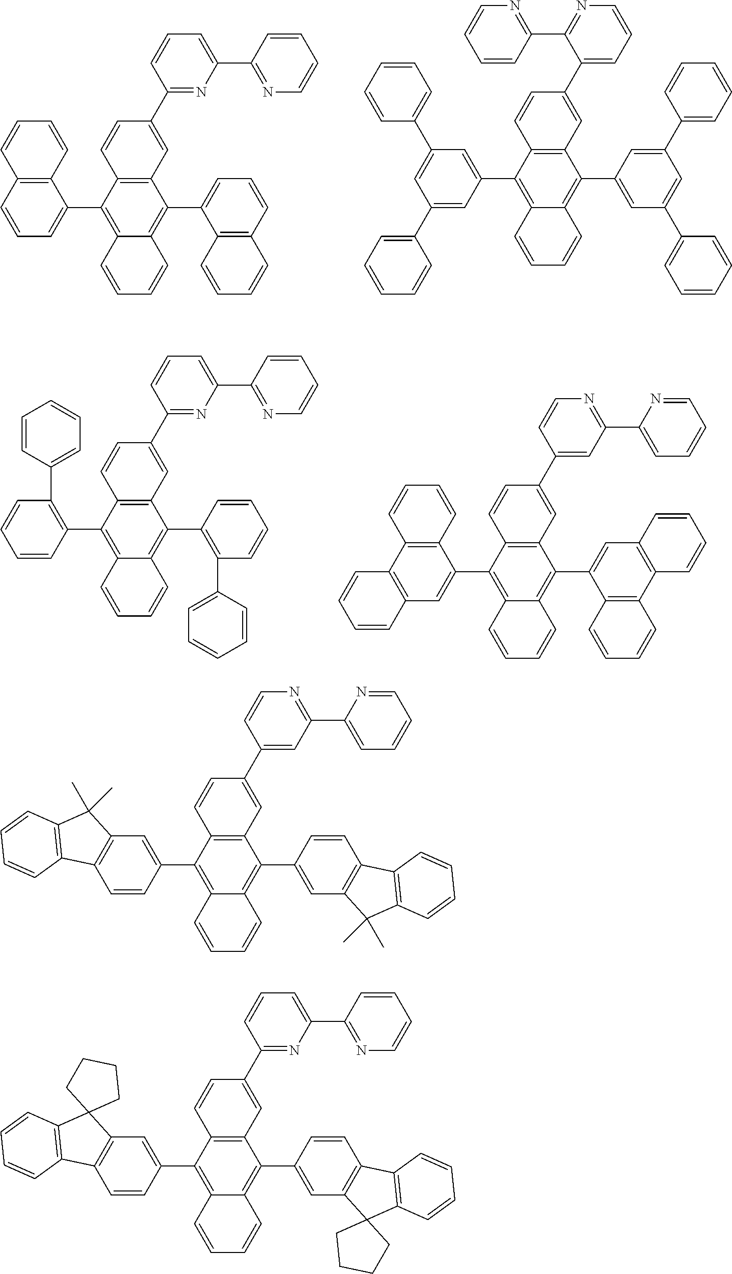

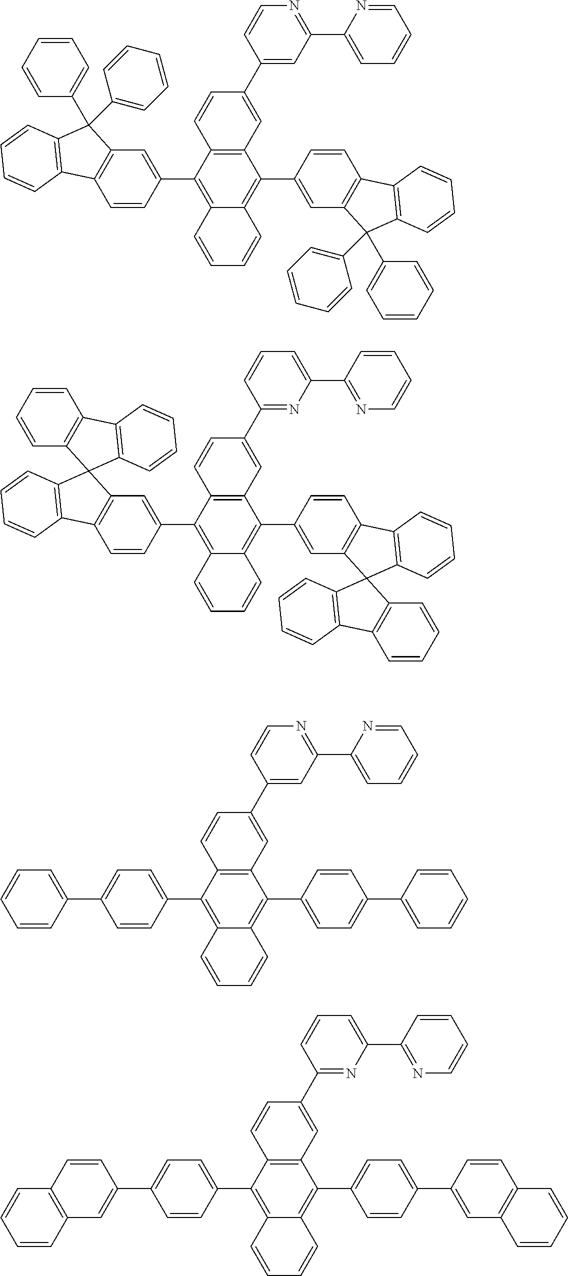

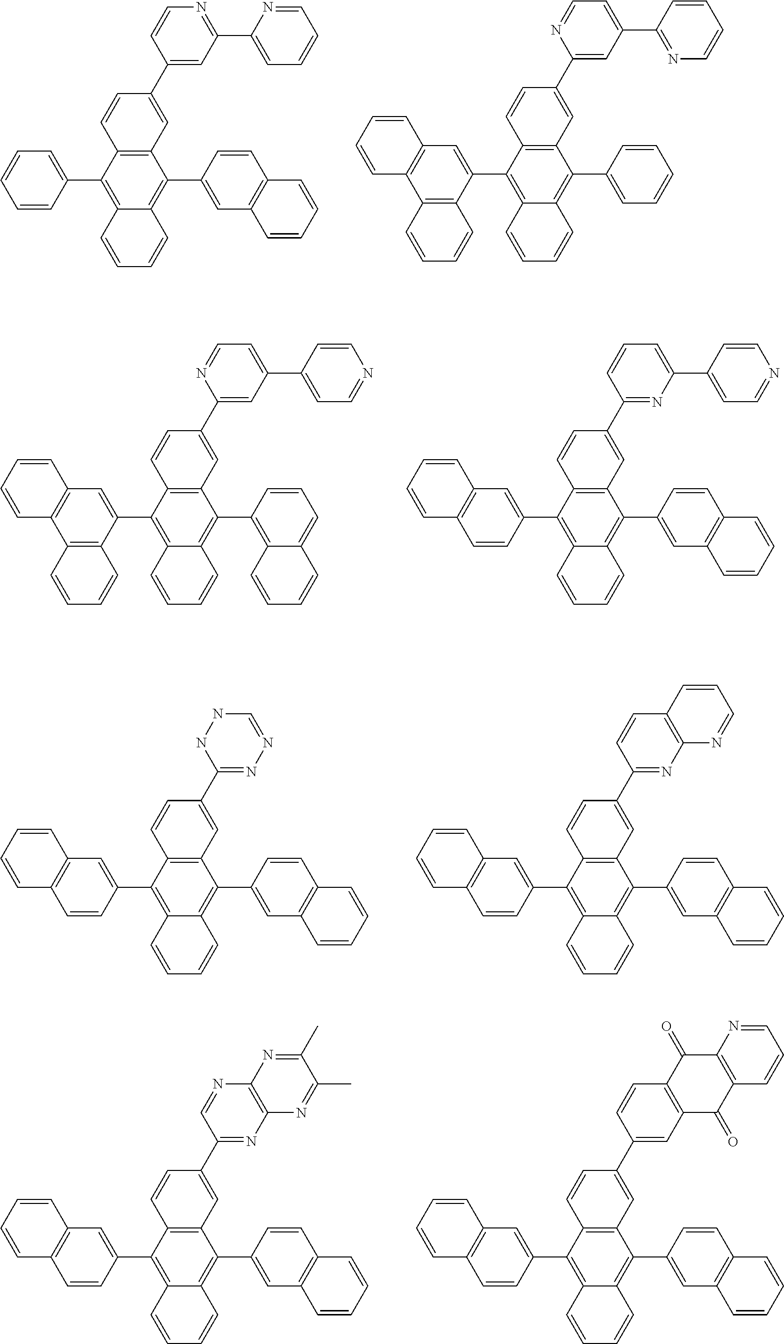

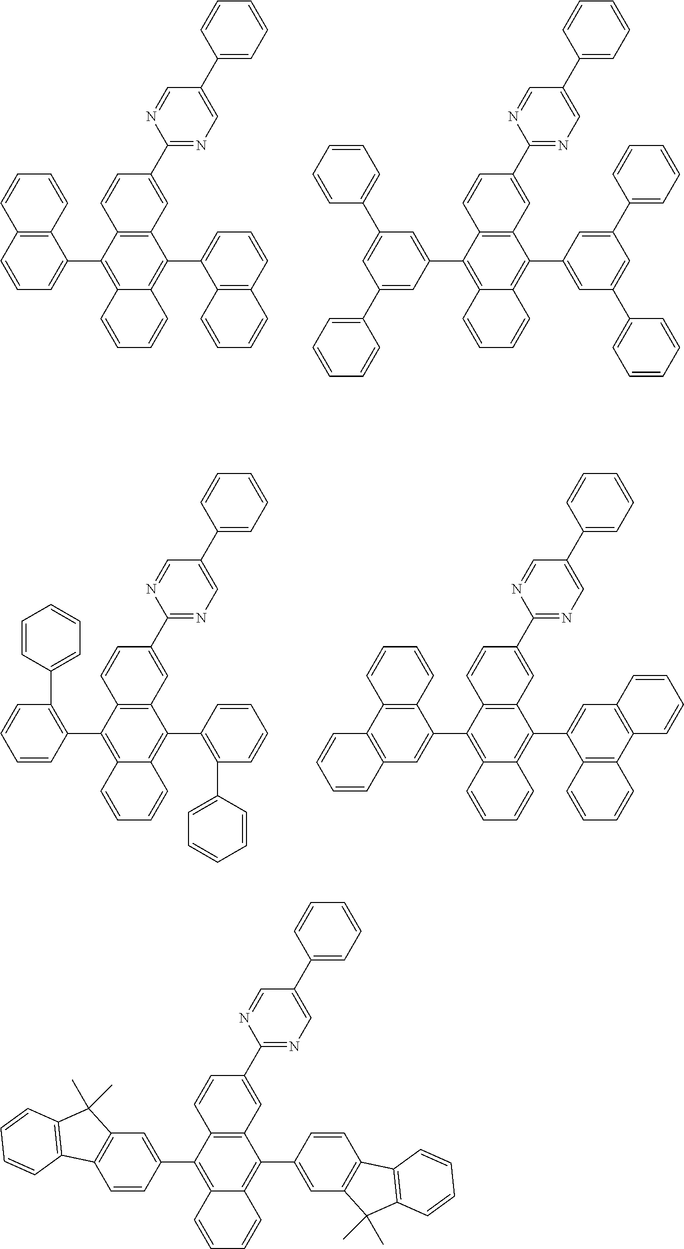 [SMILES: c1ccc(-c2cnc(-c3ccc4c(-c5cccc6ccccc56)c5ccccc5c(-c5cccc6ccccc56)c4c3)nc2)cc1 ; c1ccc(-c2cnc(-c3ccc4c(-c5cc(-c6ccccc6)cc(-c6ccccc6)c5)c5ccccc5c(-c5cc(-c6ccccc6)cc(-c6ccccc6)c5)c4c3)nc2)cc1 ; c1ccc(-c2cnc(-c3ccc4c(-c5ccccc5-c5ccccc5)c5ccccc5c(-c5ccccc5-c5ccccc5)c4c3)nc2)cc1 ; c1ccc(-c2cnc(-c3ccc4c(-c5cc6ccccc6c6ccccc56)c5ccccc5c(-c5cc6ccccc6c6ccccc56)c4c3)nc2)cc1 ; CC1(C)c2ccccc2-c2ccc(-c3c4ccccc4c(-c4ccc5c(c4)C(C)(C)c4ccccc4-5)c4cc(-c5ncc(-c6ccccc6)cn5)ccc34)cc21]

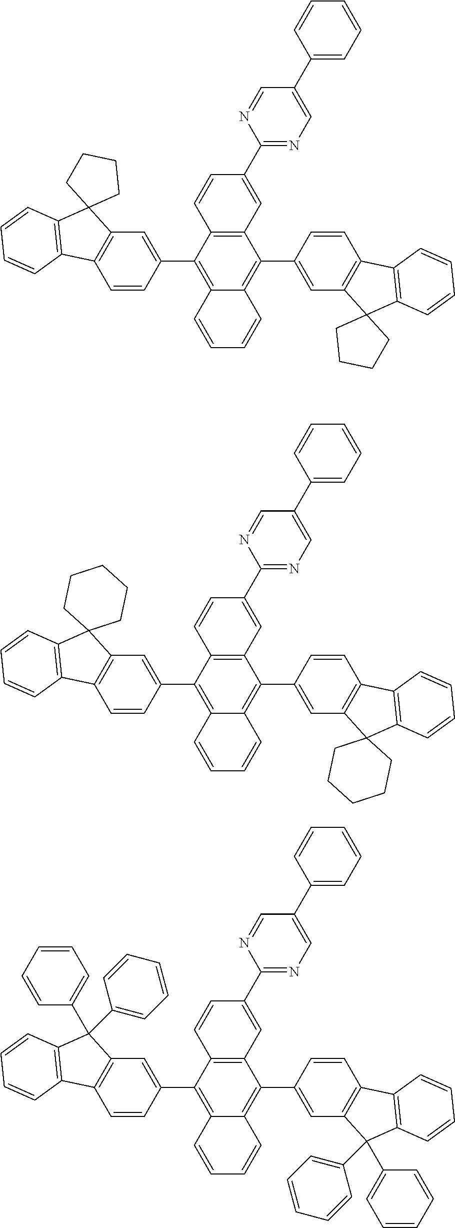

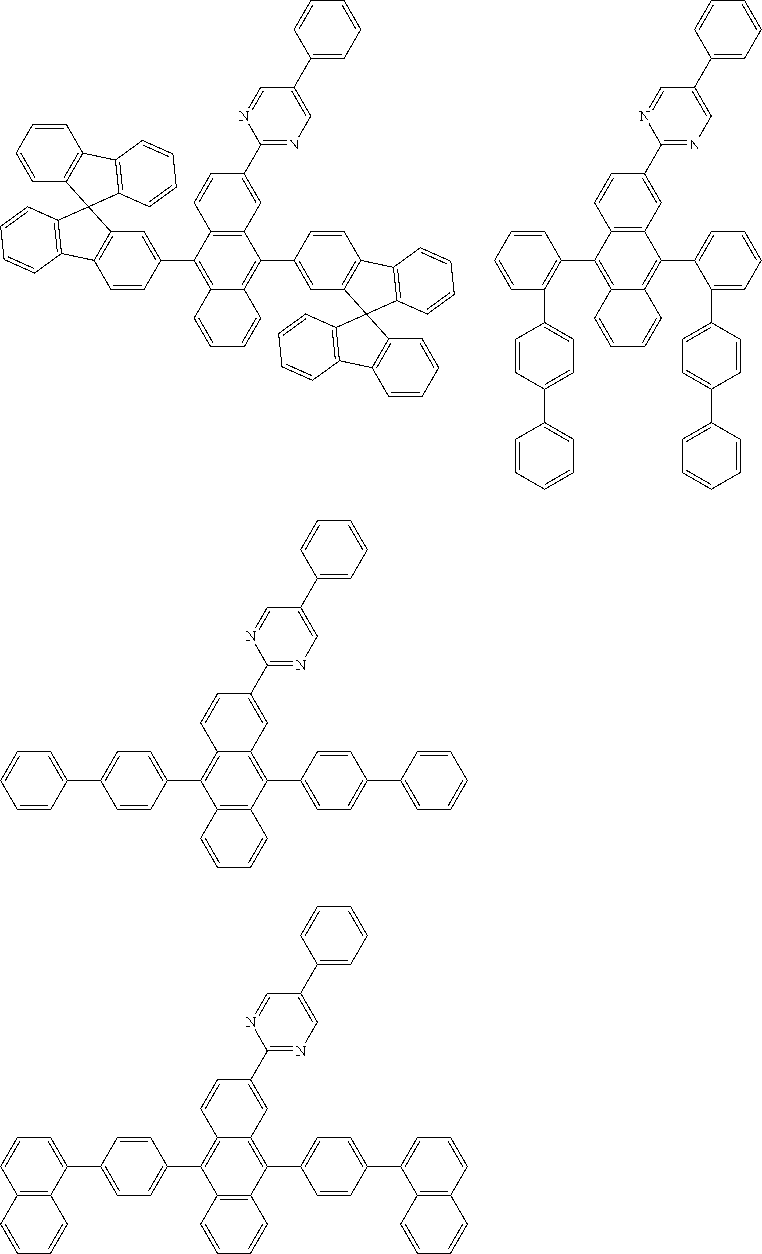

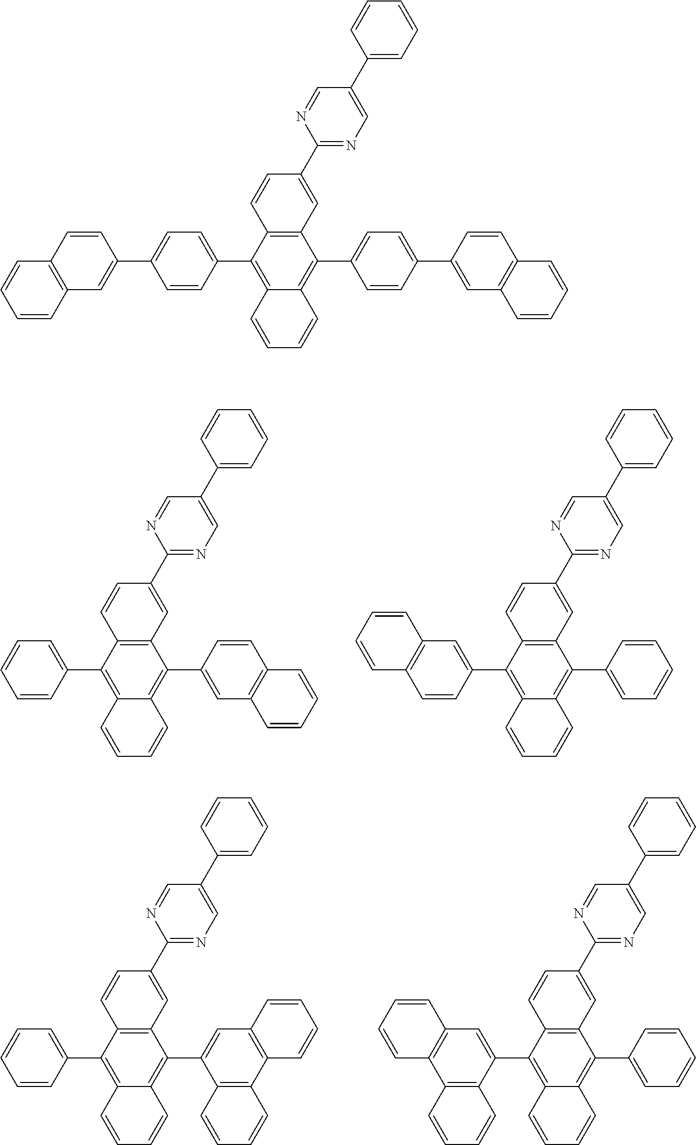 [SMILES: c1ccc(-c2cnc(-c3ccc4c(-c5ccc(-c6ccc7ccccc7c6)cc5)c5ccccc5c(-c5ccc(-c6ccc7ccccc7c6)cc5)c4c3)nc2)cc1 ; c1ccc(-c2cnc(-c3ccc4c(-c5ccccc5)c5ccccc5c(-c5ccc6ccccc6c5)c4c3)nc2)cc1 ; c1ccc(-c2cnc(-c3ccc4c(-c5ccc6ccccc6c5)c5ccccc5c(-c5ccccc5)c4c3)nc2)cc1 ; c1ccc(-c2cnc(-c3ccc4c(-c5ccccc5)c5ccccc5c(-c5cc6ccccc6c6ccccc56)c4c3)nc2)cc1 ; c1ccc(-c2cnc(-c3ccc4c(-c5cc6ccccc6c6ccccc56)c5ccccc5c(-c5ccccc5)c4c3)nc2)cc1]

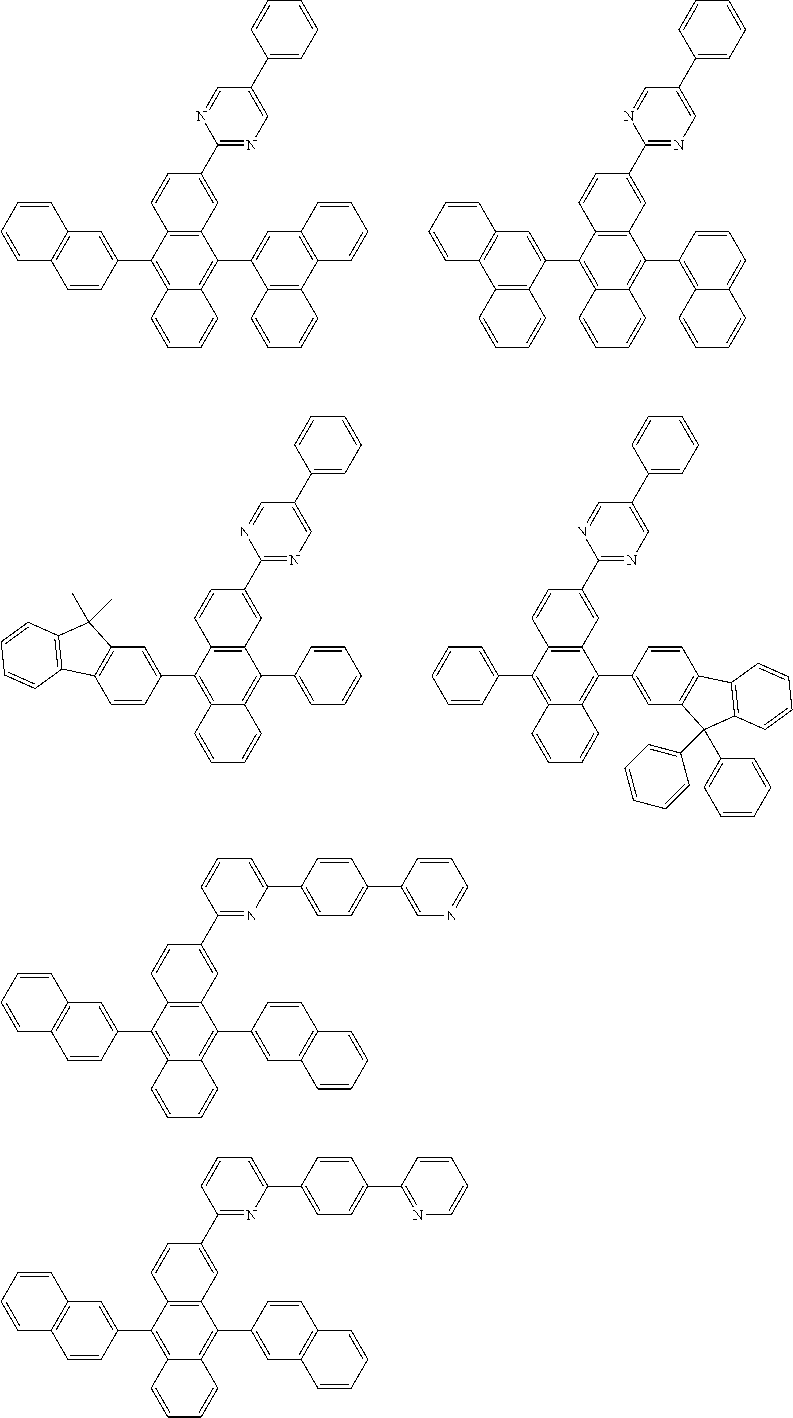

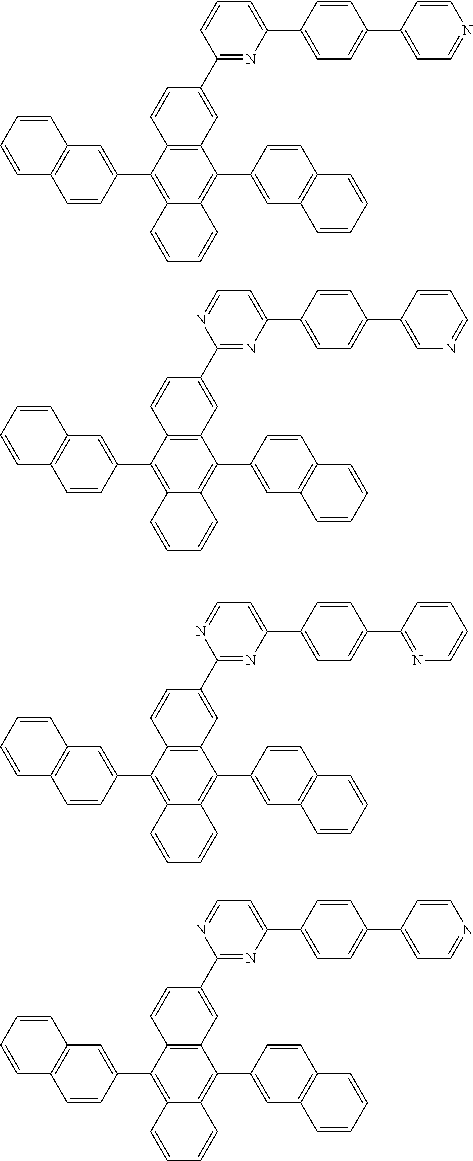

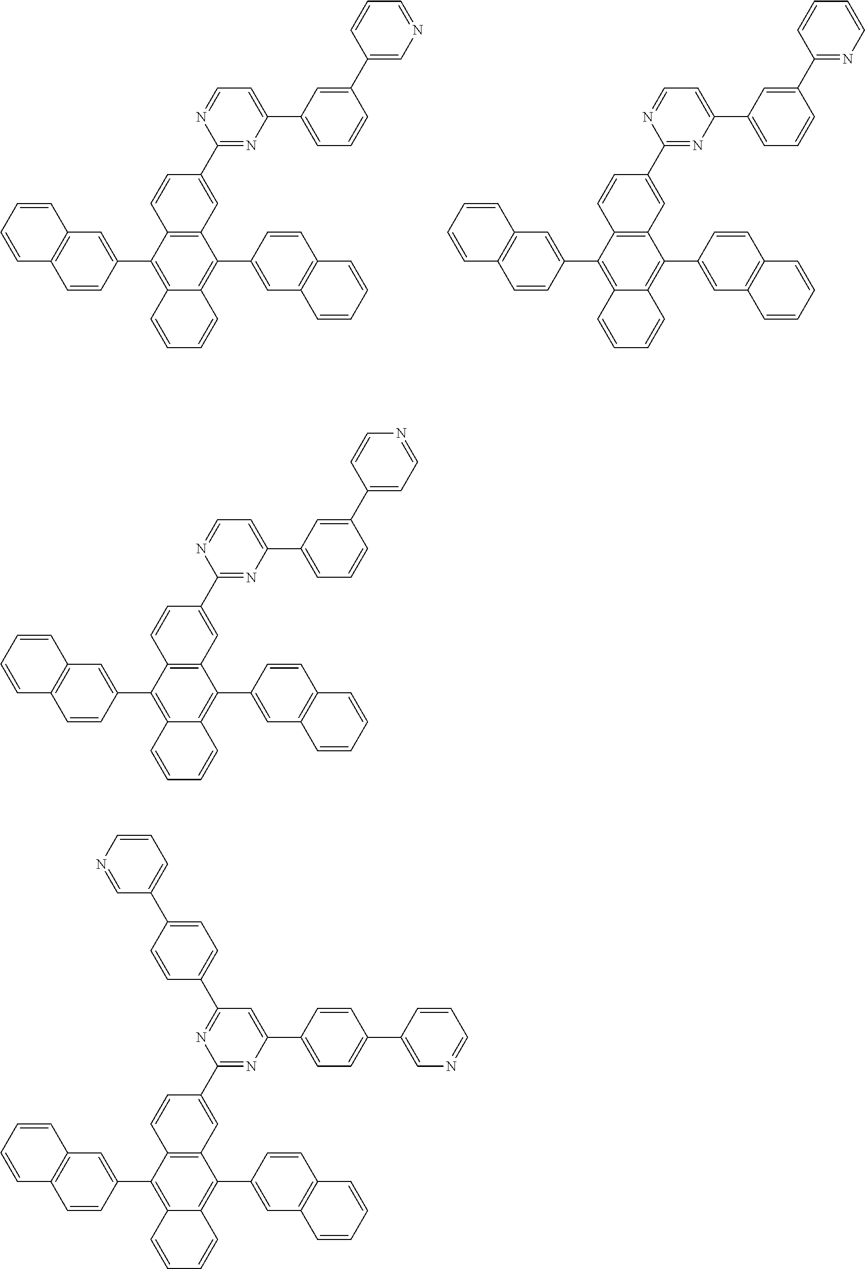 [SMILES: c1cncc(-c2cccc(-c3ccnc(-c4ccc5c(-c6ccc7ccccc7c6)c6ccccc6c(-c6ccc7ccccc7c6)c5c4)n3)c2)c1 ; c1ccc(-c2cccc(-c3ccnc(-c4ccc5c(-c6ccc7ccccc7c6)c6ccccc6c(-c6ccc7ccccc7c6)c5c4)n3)c2)nc1 ; c1cc(-c2ccncc2)cc(-c2ccnc(-c3ccc4c(-c5ccc6ccccc6c5)c5ccccc5c(-c5ccc6ccccc6c5)c4c3)n2)c1 ; c1cncc(-c2ccc(-c3cc(-c4ccc(-c5cccnc5)cc4)nc(-c4ccc5c(-c6ccc7ccccc7c6)c6ccccc6c(-c6ccc7ccccc7c6)c5c4)n3)cc2)c1]

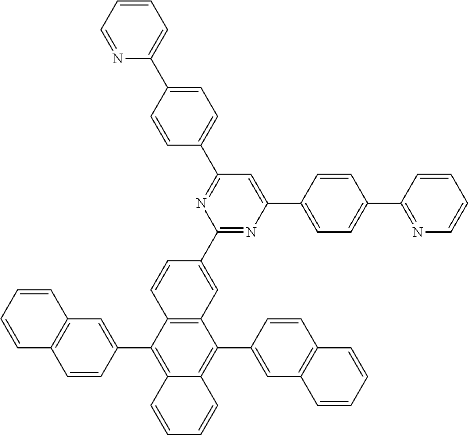 [SMILES: c1ccc(-c2ccc(-c3cc(-c4ccc(-c5ccccn5)cc4)nc(-c4ccc5c(-c6ccc7ccccc7c6)c6ccccc6c(-c6ccc7ccccc7c6)c5c4)n3)cc2)nc1]

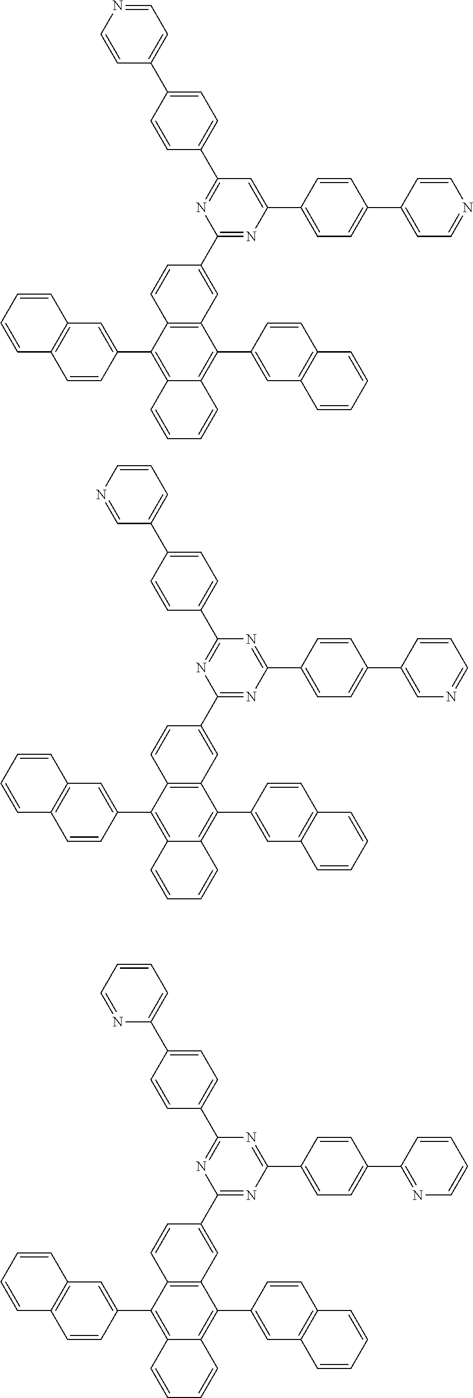

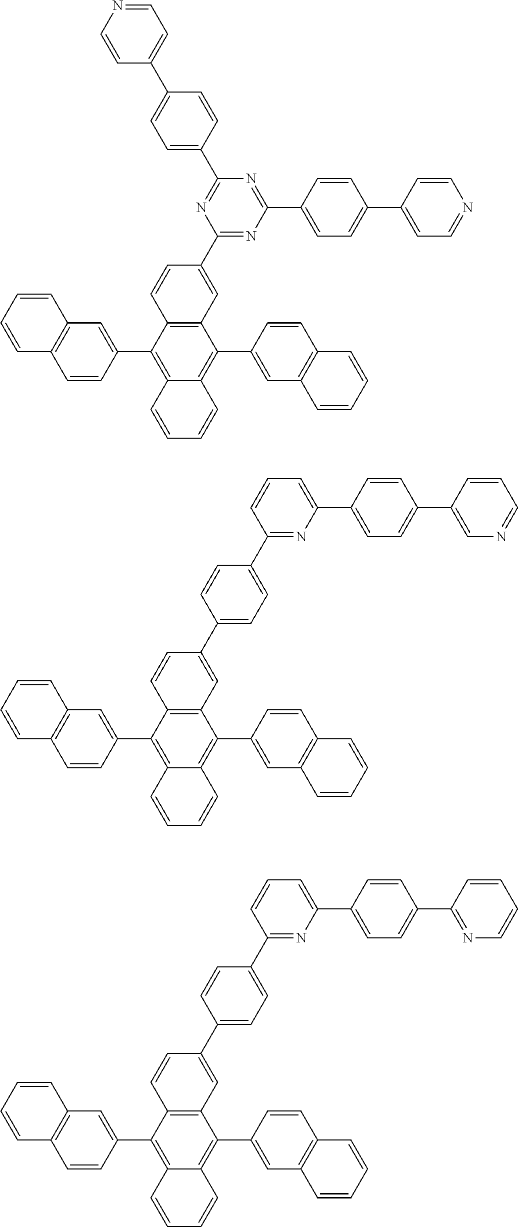 [SMILES: c1ccc2cc(-c3c4ccccc4c(-c4ccc5ccccc5c4)c4cc(-c5nc(-c6ccc(-c7ccncc7)cc6)nc(-c6ccc(-c7ccncc7)cc6)n5)ccc34)ccc2c1 ; c1cncc(-c2ccc(-c3cccc(-c4ccc(-c5ccc6c(-c7ccc8ccccc8c7)c7ccccc7c(-c7ccc8ccccc8c7)c6c5)cc4)n3)cc2)c1 ; c1ccc(-c2ccc(-c3cccc(-c4ccc(-c5ccc6c(-c7ccc8ccccc8c7)c7ccccc7c(-c7ccc8ccccc8c7)c6c5)cc4)n3)cc2)nc1]

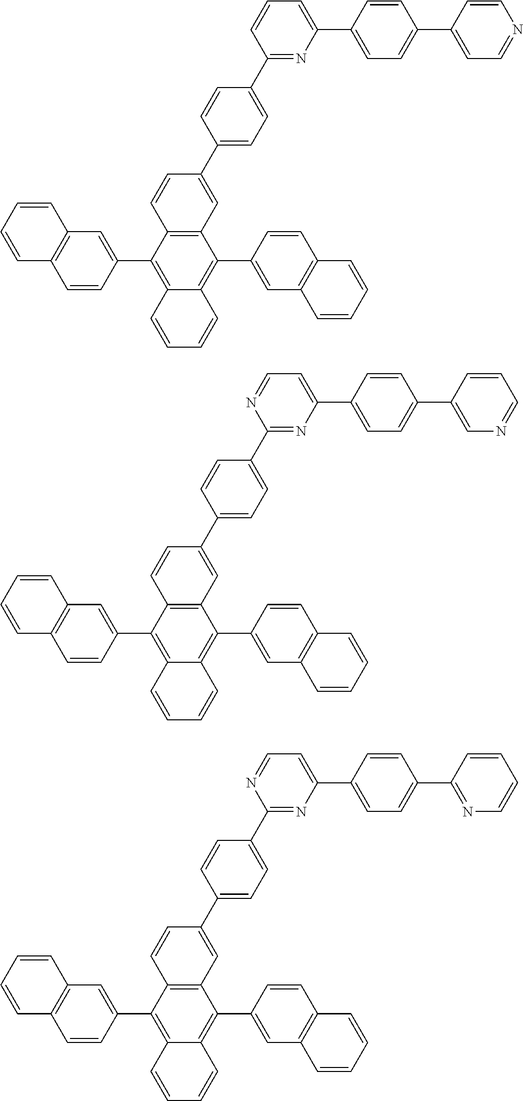 [SMILES: c1cc(-c2ccc(-c3ccncc3)cc2)nc(-c2ccc(-c3ccc4c(-c5ccc6ccccc6c5)c5ccccc5c(-c5ccc6ccccc6c5)c4c3)cc2)c1 ; c1cncc(-c2ccc(-c3ccnc(-c4ccc(-c5ccc6c(-c7ccc8ccccc8c7)c7ccccc7c(-c7ccc8ccccc8c7)c6c5)cc4)n3)cc2)c1 ; c1ccc(-c2ccc(-c3ccnc(-c4ccc(-c5ccc6c(-c7ccc8ccccc8c7)c7ccccc7c(-c7ccc8ccccc8c7)c6c5)cc4)n3)cc2)nc1]

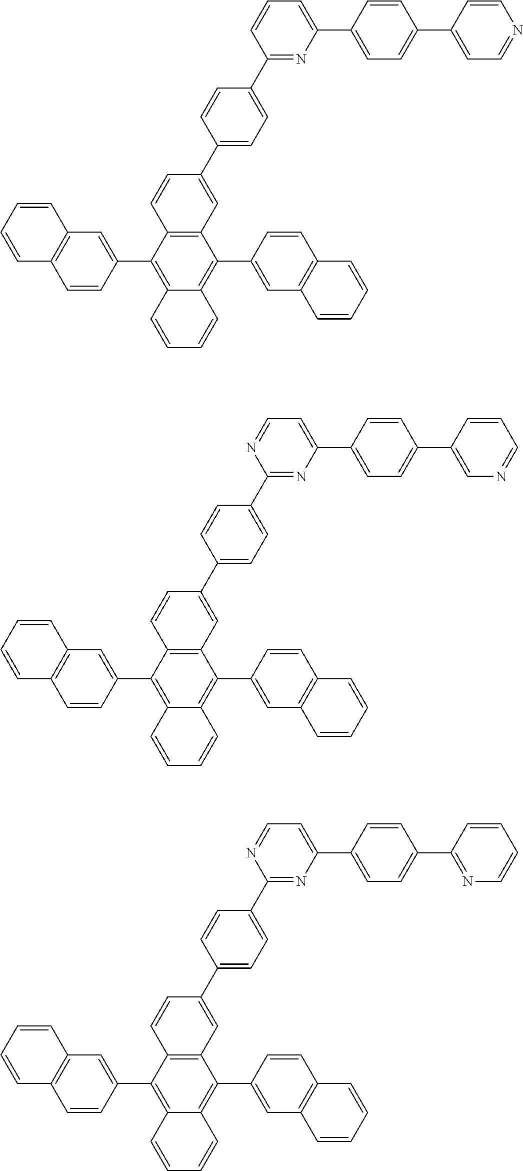 [SMILES: c1cc(-c2ccc(-c3ccncc3)cc2)nc(-c2ccc(-c3ccc4c(-c5ccc6ccccc6c5)c5ccccc5c(-c5ccc6ccccc6c5)c4c3)cc2)c1 ; c1cncc(-c2ccc(-c3ccnc(-c4ccc(-c5ccc6c(-c7ccc8ccccc8c7)c7ccccc7c(-c7ccc8ccccc8c7)c6c5)cc4)n3)cc2)c1 ; c1ccc(-c2ccc(-c3ccnc(-c4ccc(-c5ccc6c(-c7ccc8ccccc8c7)c7ccccc7c(-c7ccc8ccccc8c7)c6c5)cc4)n3)cc2)nc1]

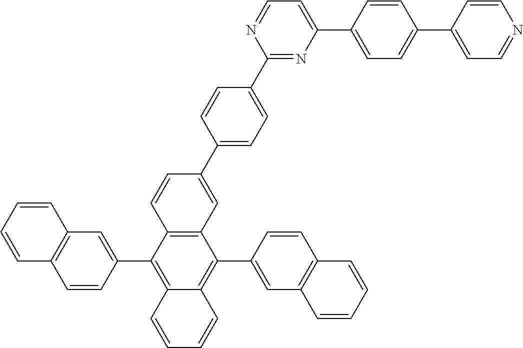 [SMILES: c1ccc2cc(-c3c4ccccc4c(-c4ccc5ccccc5c4)c4cc(-c5ccc(-c6nccc(-c7ccc(-c8ccncc8)cc7)n6)cc5)ccc34)ccc2c1]

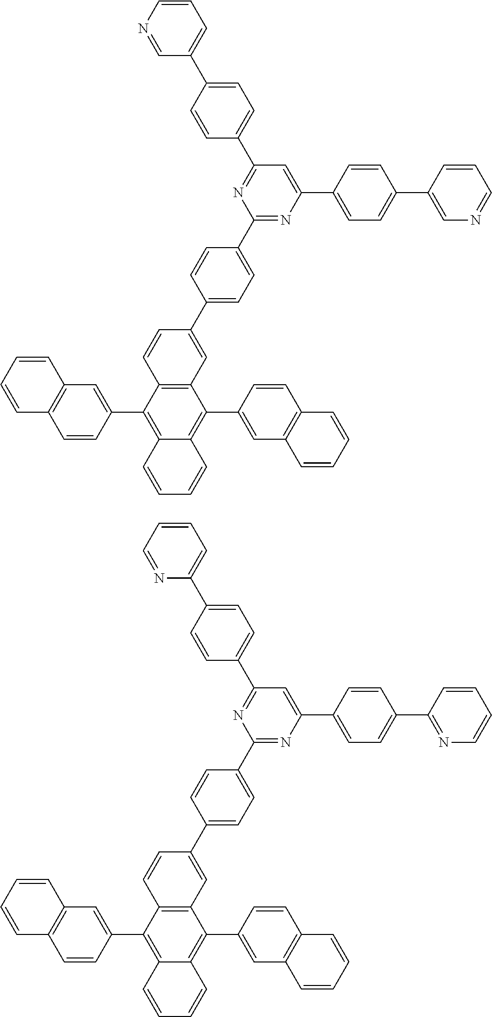

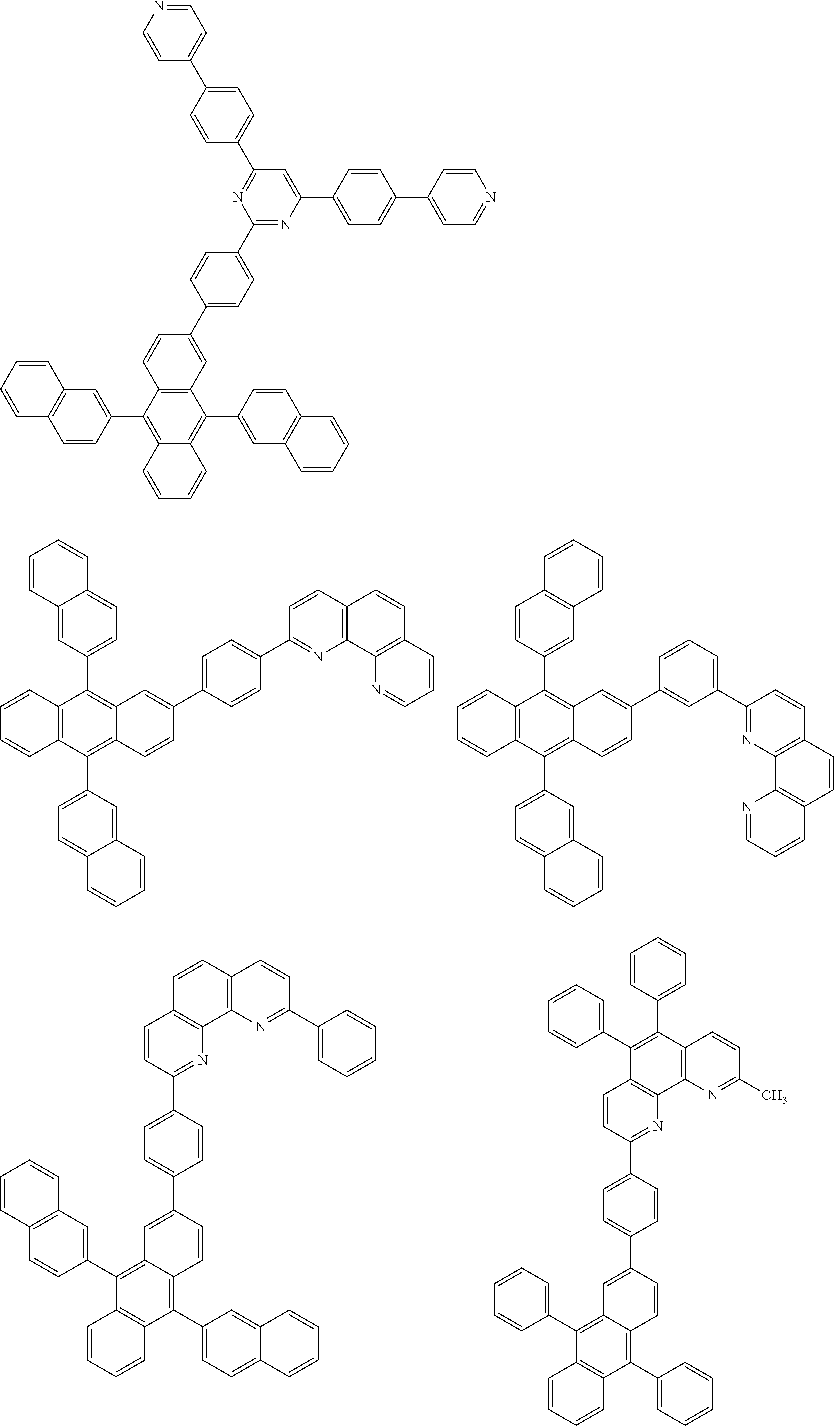

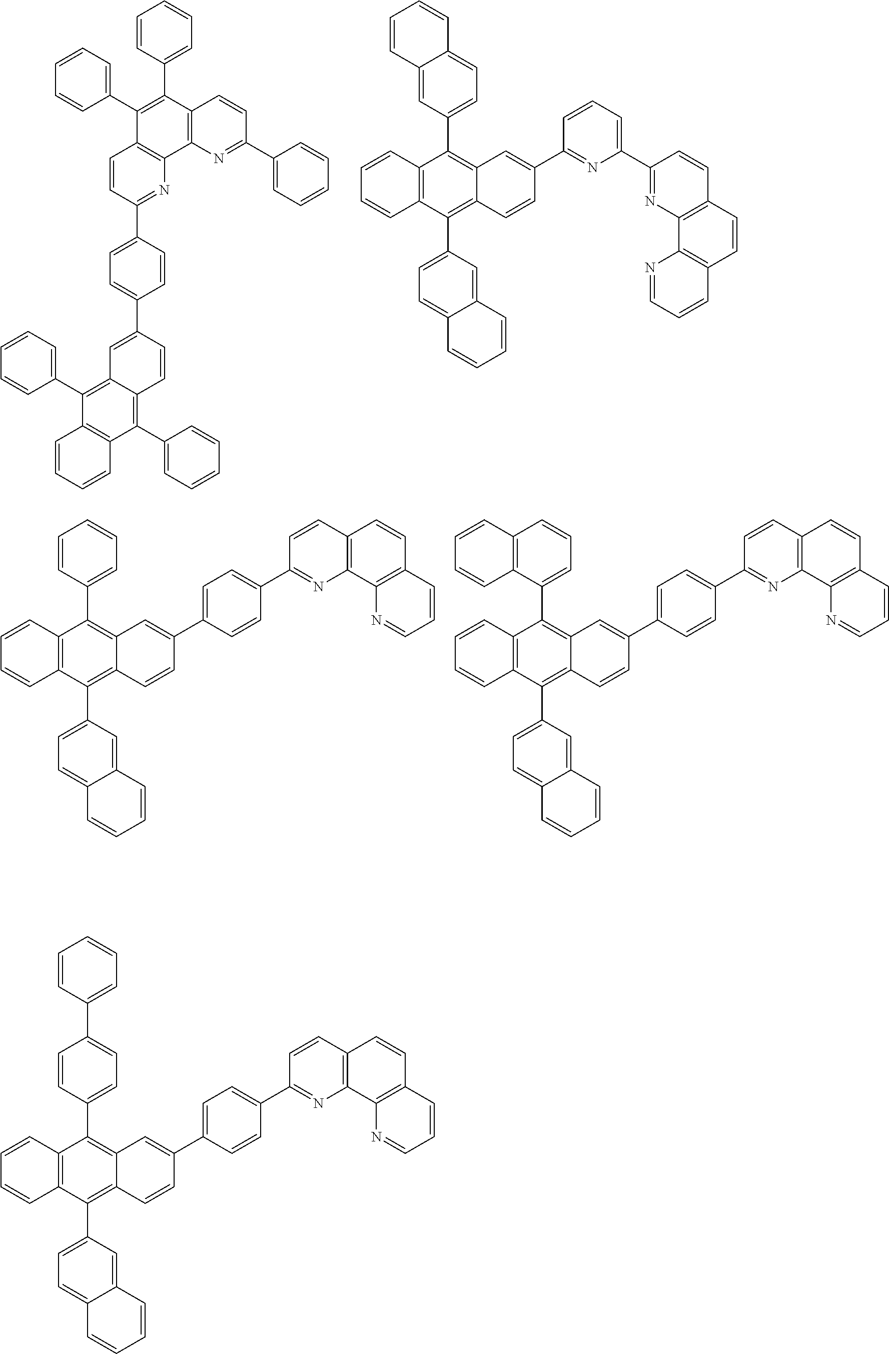

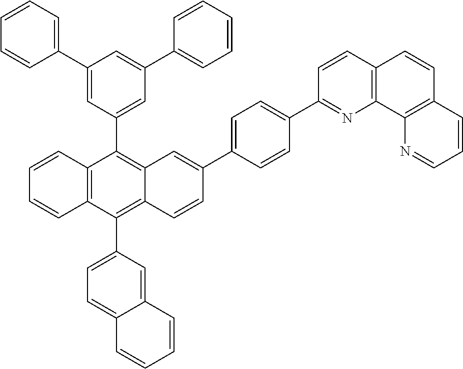 [SMILES: c1ccc(-c2cc(-c3ccccc3)cc(-c3c4ccccc4c(-c4ccc5ccccc5c4)c4ccc(-c5ccc(-c6ccc7ccc8cccnc8c7n6)cc5)cc34)c2)cc1]

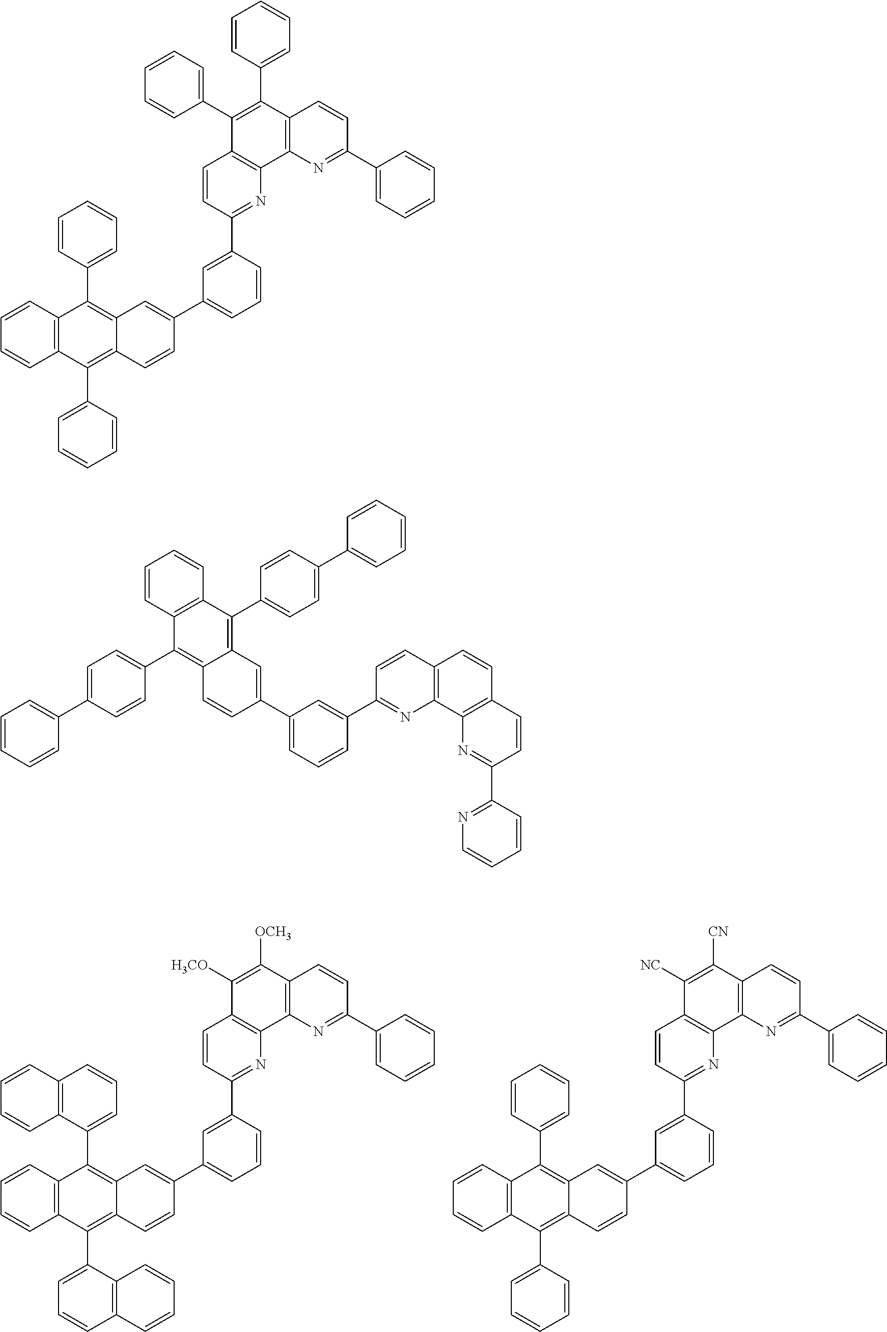 [SMILES: c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1 ; c1ccc(-c2ccc(-c3c4ccccc4c(-c4ccc(-c5ccccc5)cc4)c4cc(-c5cccc(-c6ccc7ccc8ccc(-c9ccccn9)nc8c7n6)c5)ccc34)cc2)cc1 ; COc1c(OC)c2ccc(-c3cccc(-c4ccc5c(-c6cccc7ccccc67)c6ccccc6c(-c6cccc7ccccc67)c5c4)c3)nc2c2nc(-c3ccccc3)ccc12 ; N#Cc1c(C#N)c2ccc(-c3cccc(-c4ccc5c(-c6ccccc6)c6ccccc6c(-c6ccccc6)c5c4)c3)nc2c2nc(-c3ccccc3)ccc12]

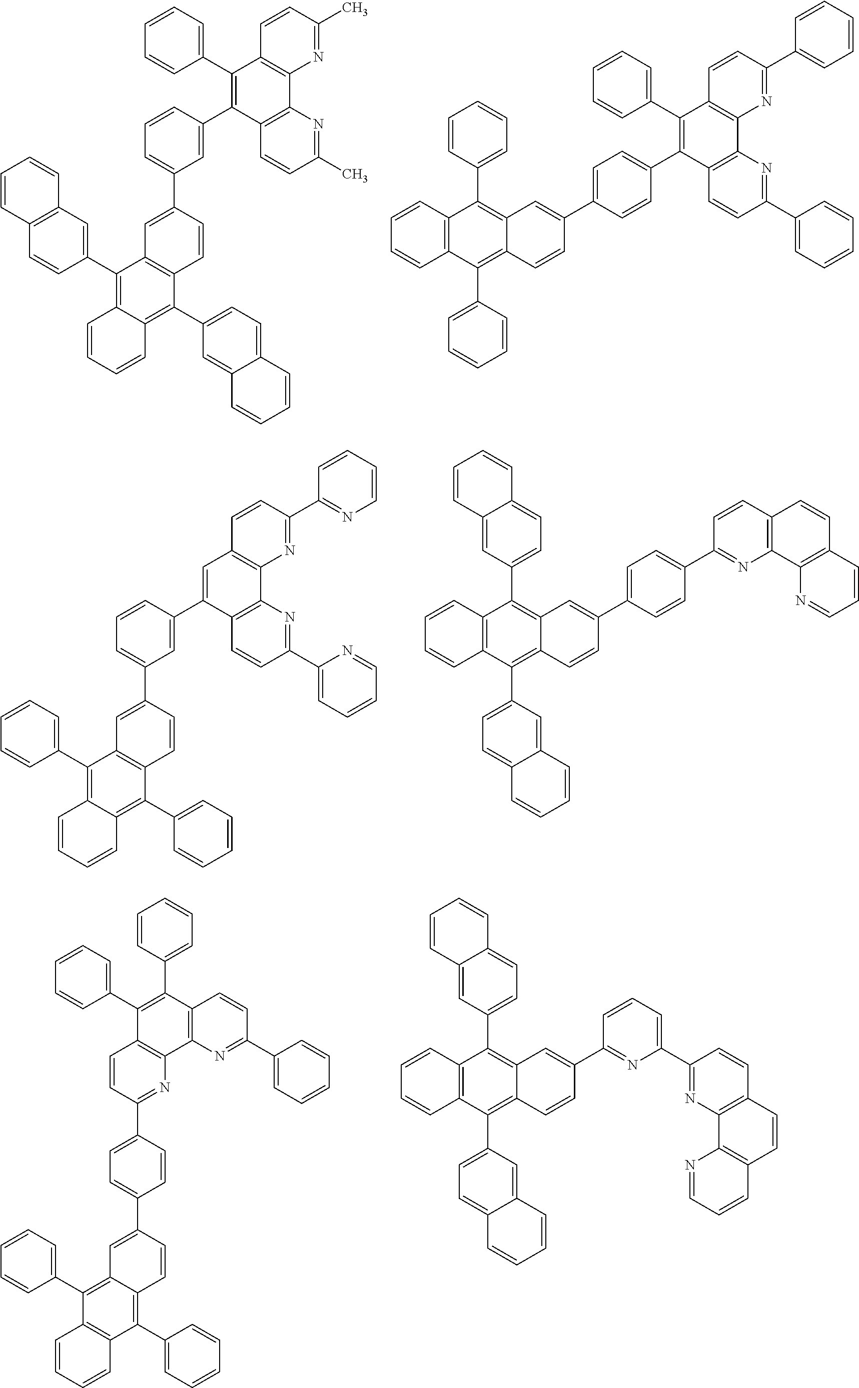

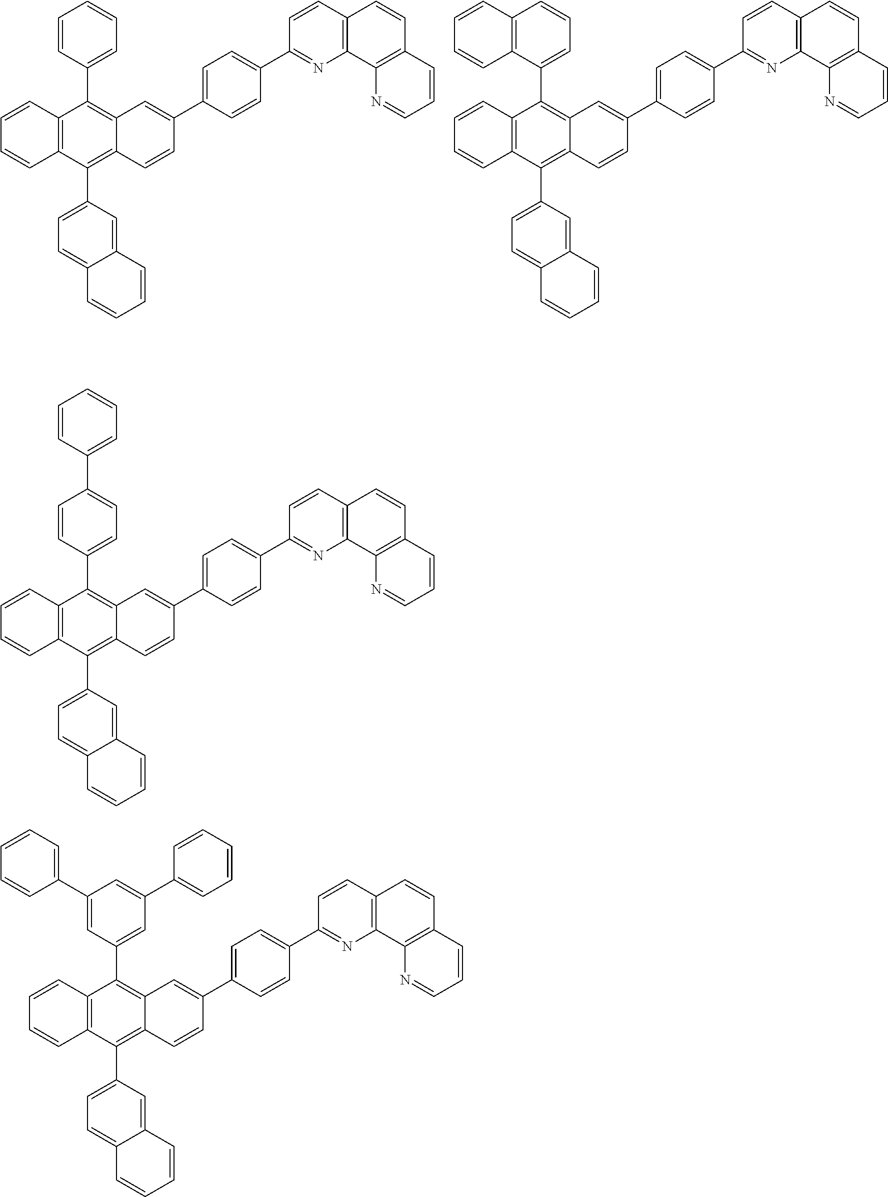

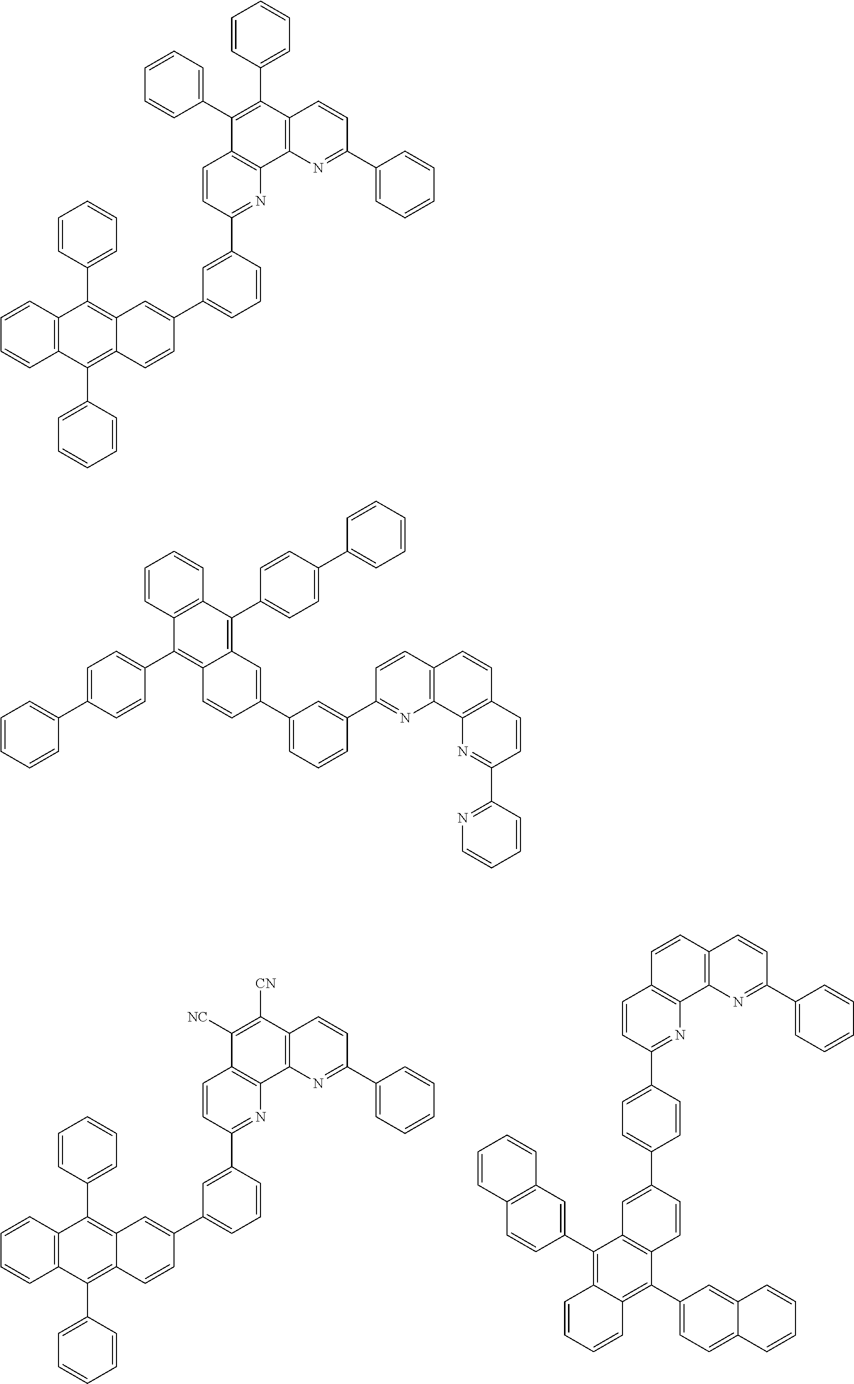 [SMILES: c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1 ; c1ccc(-c2ccc(-c3c4ccccc4c(-c4ccc(-c5ccccc5)cc4)c4cc(-c5cccc(-c6ccc7ccc8ccc(-c9ccccn9)nc8c7n6)c5)ccc34)cc2)cc1 ; N#Cc1c(C#N)c2ccc(-c3cccc(-c4ccc5c(-c6ccccc6)c6ccccc6c(-c6ccccc6)c5c4)c3)nc2c2nc(-c3ccccc3)ccc12 ; c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1]

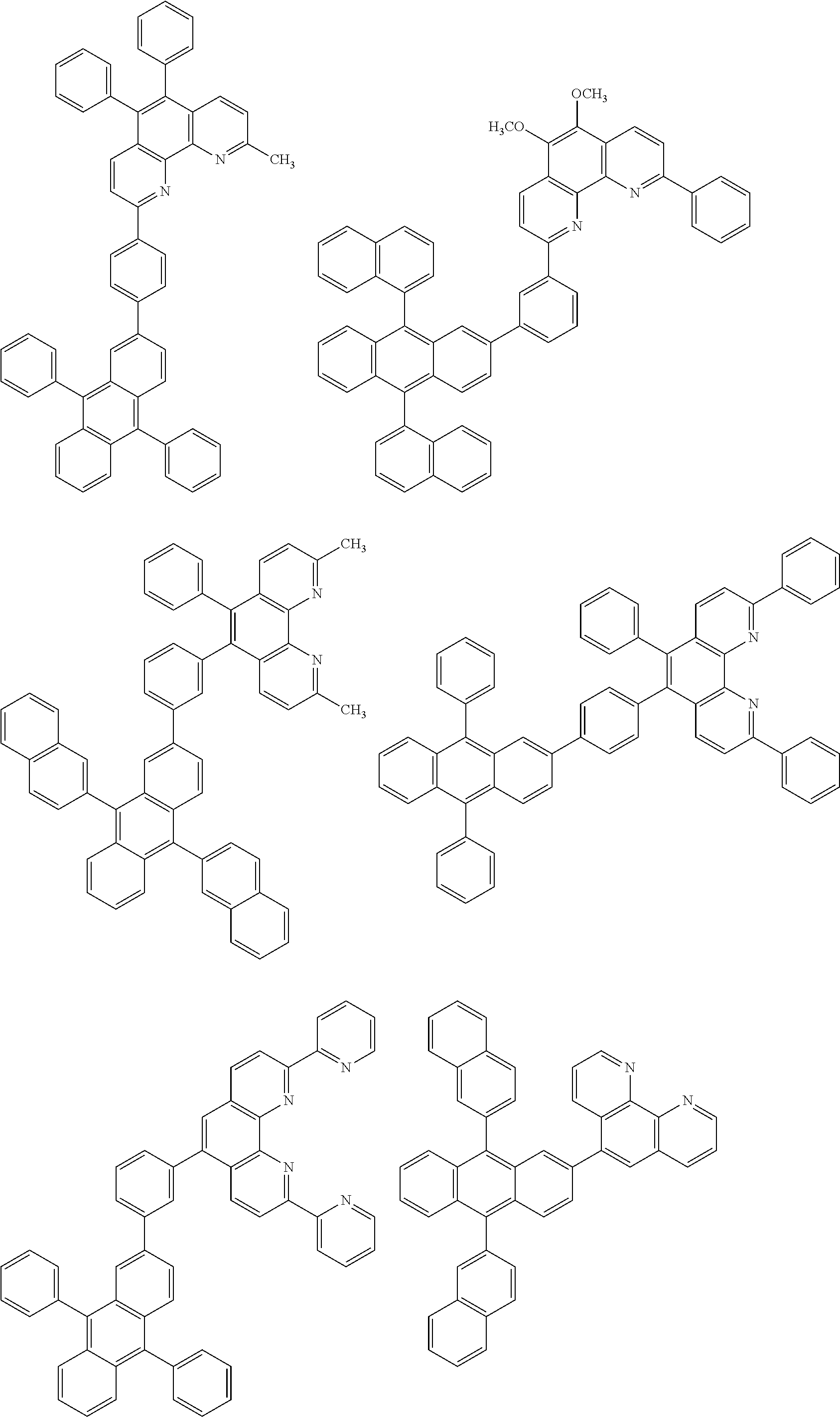

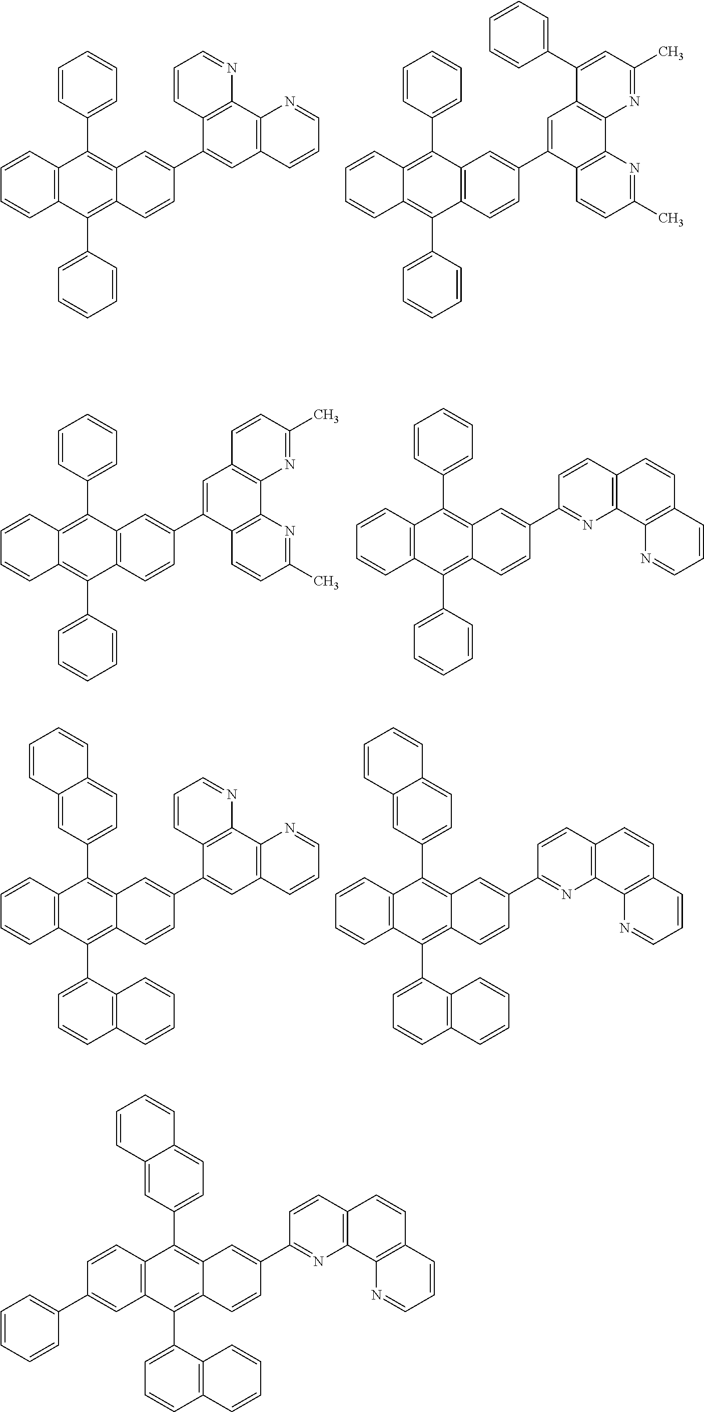

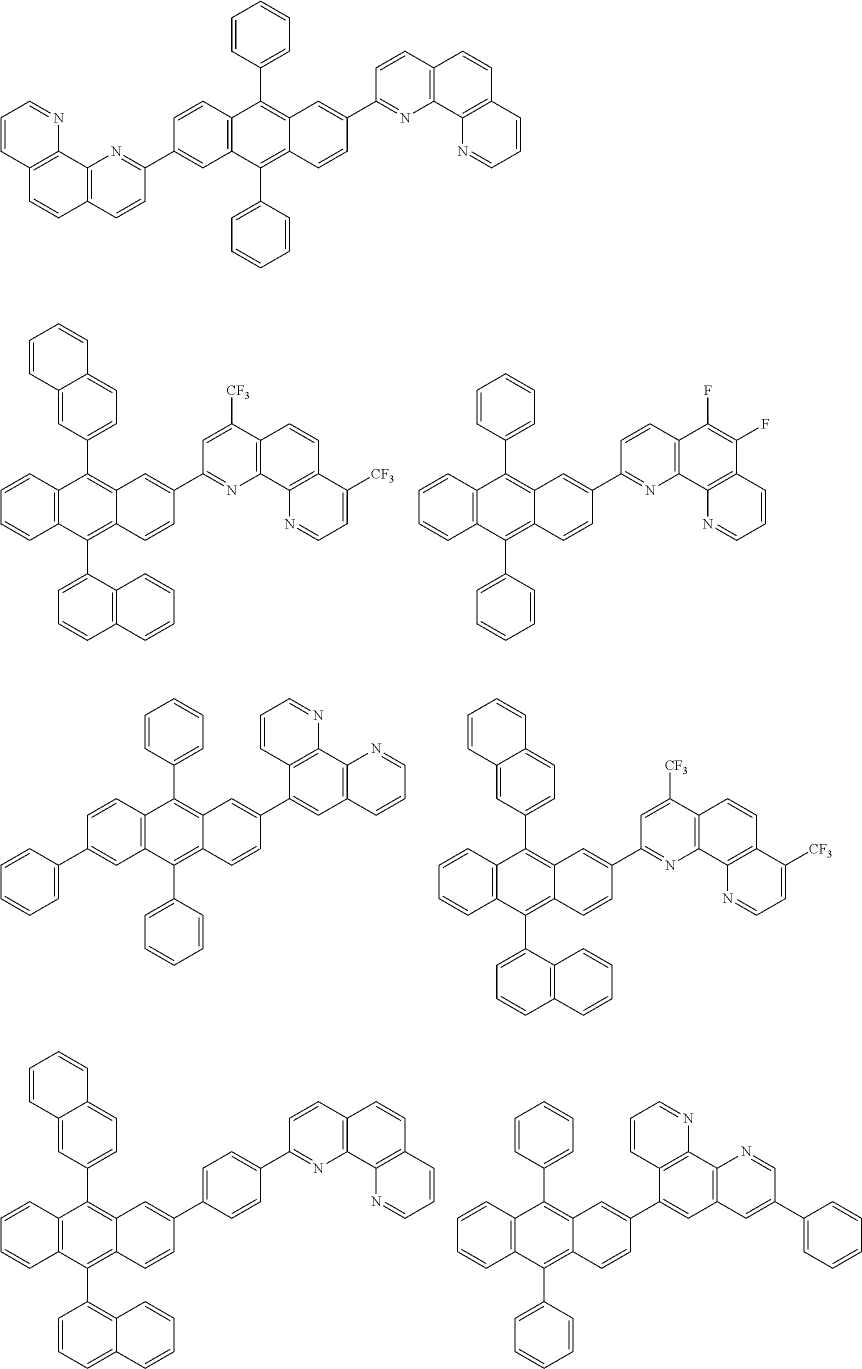

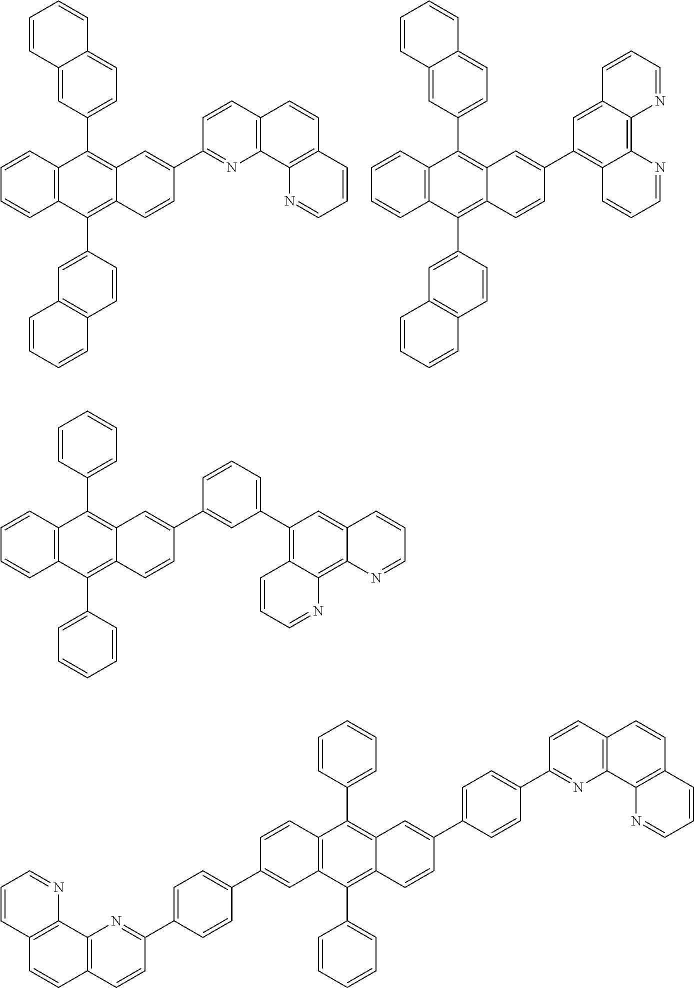

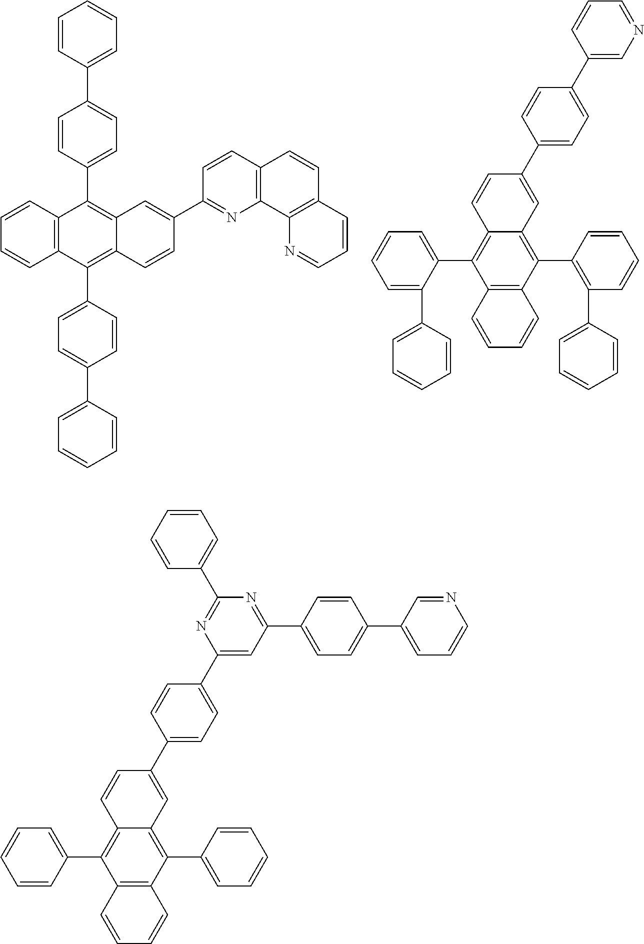

### [Compound Represented by Formula (2)]

A compound represented by the formula (2) is shown as follows:

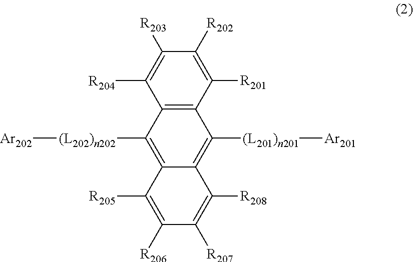

wherein in the formula (2),
R₂₀₁ to R₂₀₈ are independently a hydrogen atom, or a substituent R;
L₂₀₁ and L₂₀₂ are independently
a single bond, or
a substituted or unsubstituted arylene group having 6 to 50 ring carbon atoms;
Ar₂₀₁ and Ar₂₀₂ are independently a substituted or unsubstituted monovalent heterocyclic group having 5 to 50 ring atoms;
n201 is an integer of 1 to 3; when n201 is 2 or more, two or more L₂₀₁'s may be the same as or different from each other;
n202 is an integer of 1 to 3; when n202 is 2 or more, two or more L₂₀₂'s may be the same as or different from each other; and
the substituent R is the same as defined in the formula (1).

When all of L₂₀₁'s is a single bond in the formula (2), Ar₂₀₁ is directly bonded with the anthracene skeleton.

When all of L₂₀₂'s is a single bond in the formula (2), Ar₂₀₂ is directly bonded with the anthracene skeleton.

In one embodiment, the compound represented by the formula (2) is a compound represented by the following formula (2-1):

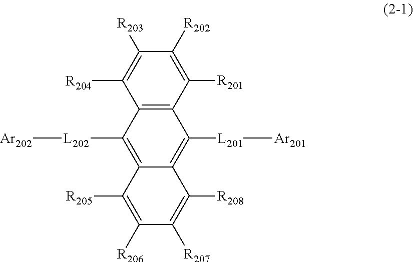

wherein in the formula (2-1),
R₂₀₁ to R₂₀₈, L₂₀₁, L₂₀₂, Ar₂₀₁, and Ar₂₀₂ are the same as defined in the formula (2).

In one embodiment, Ar₂₀₁ is a substituted or unsubstituted benzimidazolyl group.

In one embodiment, R₂₀₁ to R₂₀₅ are hydrogen atoms.

In one embodiment, the compound represented by the formula (2) is a compound represented by the following formula (2-11):

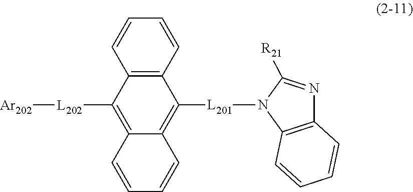

wherein in the formula (2-11),
L₂₀₁, L₂₀₂, and Ar₂₀₂ are the same as defined in the formula (2);
R₂₁₁ is a hydrogen atom, or a substituent R; and
the substituent R is the same as defined in the formula (1).

In one embodiment, a substituent in the case of “substituted or unsubstituted” in the formula (2) is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,
—Si(R₄₁)(R₄₂)(R₄₃),
—C(═O)R₄₄, —COOR₄₅,
—S(═O)₂R₄₆,
—P(═O)(R₄₇)(R₄₃),
—Ge(R₄₉)(R₅₀)(R₅₁),
—N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other),
a hydroxy group,
a halogen atom,
a cyano group,
a nitro group,
an aryl group having 6 to 50 ring carbon atoms, and a monovalent heterocyclic group having 5 to 50 ring atoms.

In one embodiment, a substituent in the case of “substituted or unsubstituted” in the formula (2) is selected from the group consisting of
an alkyl group having 1 to 18 carbon atoms,
an aryl group having 6 to 18 ring carbon atoms, and
a monovalent heterocyclic group having 5 to 18 ring atoms.

The compound represented by the formula (2) can be synthesized by using known alternative reactions or raw materials adapted to the target compound.

Specific examples of the compound represented by the formula (2) will be described below, but these are merely examples, and the compound represented by the formula (2) is not limited to the following specific examples.

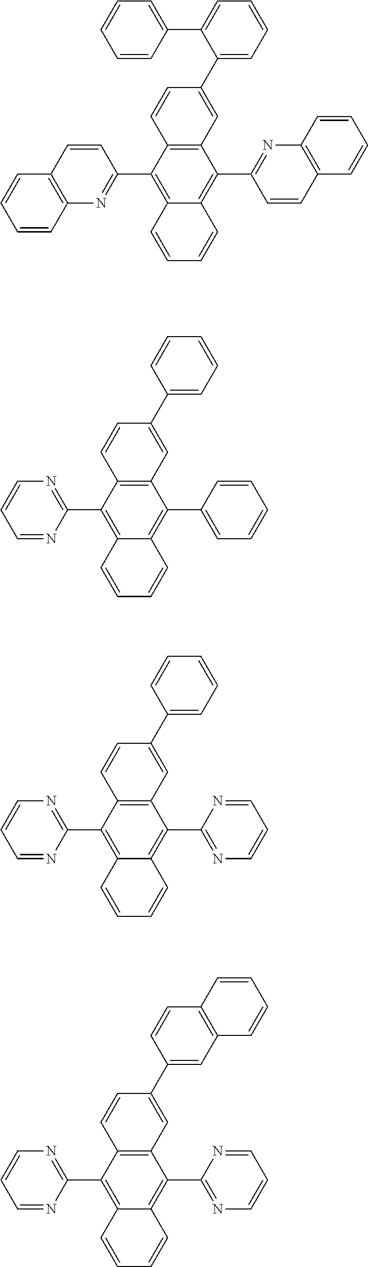 [SMILES: c1ccc(-c2ccccc2-c2ccc3c(-c4ccc5ccccc5n4)c4ccccc4c(-c4ccc5ccccc5n4)c3c2)cc1 ; c1ccc(-c2ccc3c(-c4ncccn4)c4ccccc4c(-c4ccccc4)c3c2)cc1 ; c1ccc(-c2ccc3c(-c4ncccn4)c4ccccc4c(-c4ncccn4)c3c2)cc1 ; c1cnc(-c2c3ccccc3c(-c3ncccn3)c3cc(-c4ccc5ccccc5c4)ccc23)nc1]

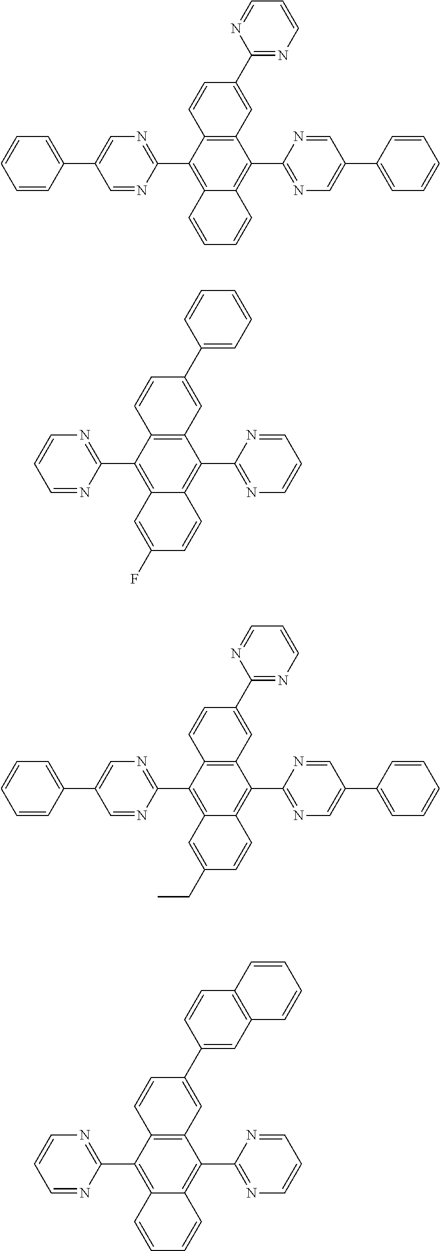 [SMILES: c1ccc(-c2cnc(-c3c4ccccc4c(-c4ncc(-c5ccccc5)cn4)c4cc(-c5ncccn5)ccc34)nc2)cc1 ; Fc1ccc2c(-c3ncccn3)c3cc(-c4ccccc4)ccc3c(-c3ncccn3)c2c1 ; CCc1ccc2c(-c3ncc(-c4ccccc4)cn3)c3cc(-c4ncccn4)ccc3c(-c3ncc(-c4ccccc4)cn3)c2c1 ; c1cnc(-c2c3ccccc3c(-c3ncccn3)c3cc(-c4ccc5ccccc5c4)ccc23)nc1]

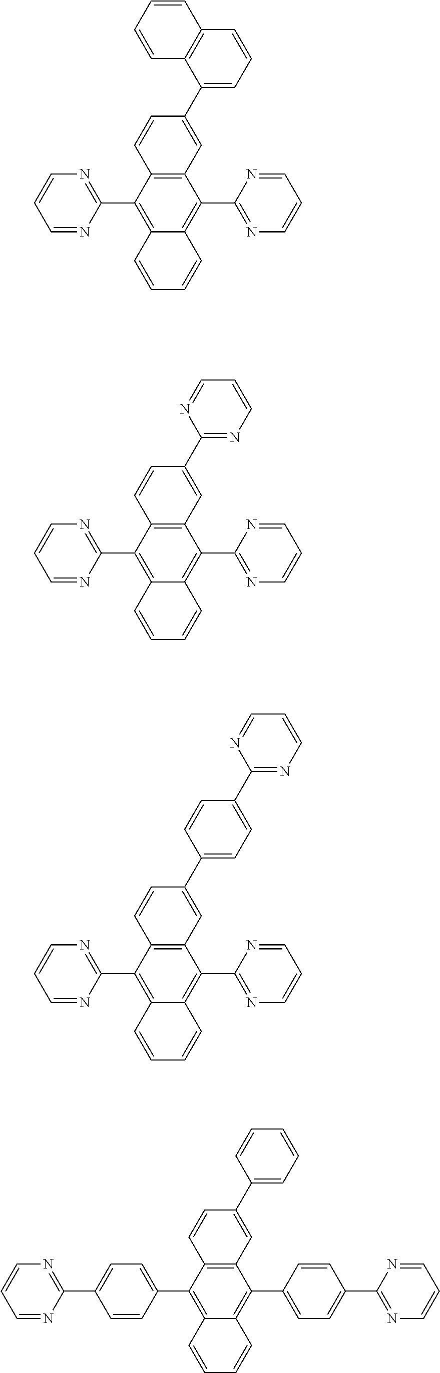 [SMILES: c1cnc(-c2c3ccccc3c(-c3ncccn3)c3cc(-c4cccc5ccccc45)ccc23)nc1 ; c1cnc(-c2ccc3c(-c4ncccn4)c4ccccc4c(-c4ncccn4)c3c2)nc1 ; c1cnc(-c2ccc(-c3ccc4c(-c5ncccn5)c5ccccc5c(-c5ncccn5)c4c3)cc2)nc1 ; c1ccc(-c2ccc3c(-c4ccc(-c5ncccn5)cc4)c4ccccc4c(-c4ccc(-c5ncccn5)cc4)c3c2)cc1]

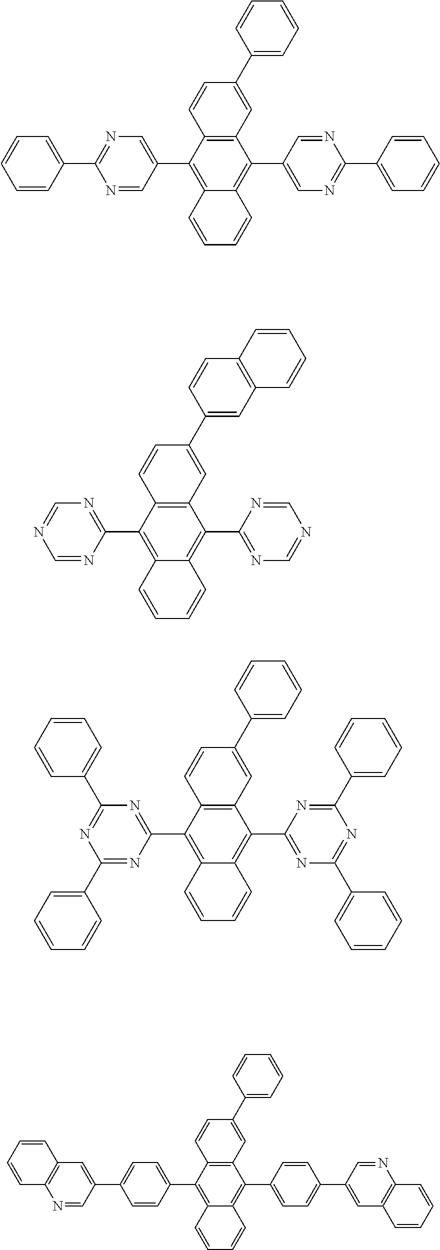 [SMILES: c1ccc(-c2ccc3c(-c4cnc(-c5ccccc5)nc4)c4ccccc4c(-c4cnc(-c5ccccc5)nc4)c3c2)cc1 ; c1ccc2cc(-c3ccc4c(-c5ncncn5)c5ccccc5c(-c5ncncn5)c4c3)ccc2c1 ; c1ccc(-c2ccc3c(-c4nc(-c5ccccc5)nc(-c5ccccc5)n4)c4ccccc4c(-c4nc(-c5ccccc5)nc(-c5ccccc5)n4)c3c2)cc1 ; c1ccc(-c2ccc3c(-c4ccc(-c5cnc6ccccc6c5)cc4)c4ccccc4c(-c4ccc(-c5cnc6ccccc6c5)cc4)c3c2)cc1]

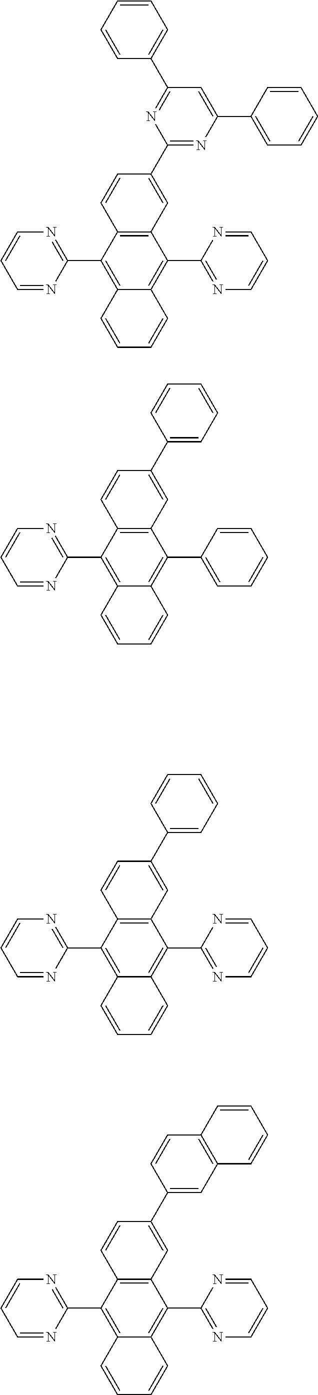

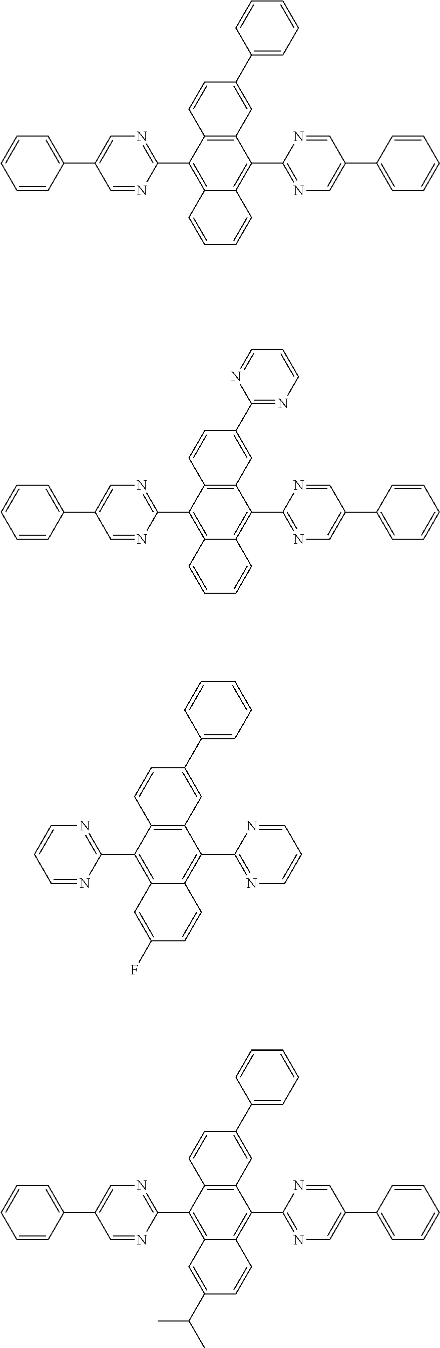 [SMILES: c1ccc(-c2cnc(-c3c4ccccc4c(-c4ncc(-c5ccccc5)cn4)c4cc(-c5ccccc5)ccc34)nc2)cc1 ; c1ccc(-c2cnc(-c3c4ccccc4c(-c4ncc(-c5ccccc5)cn4)c4cc(-c5ncccn5)ccc34)nc2)cc1 ; Fc1ccc2c(-c3ncccn3)c3cc(-c4ccccc4)ccc3c(-c3ncccn3)c2c1 ; CC(C)c1ccc2c(-c3ncc(-c4ccccc4)cn3)c3cc(-c4ccccc4)ccc3c(-c3ncc(-c4ccccc4)cn3)c2c1]

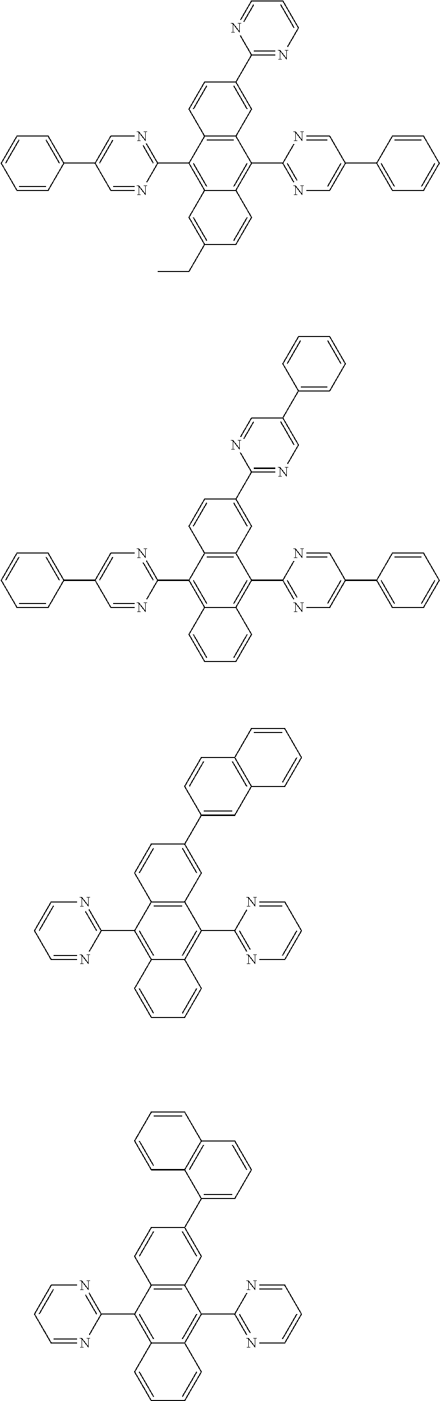 [SMILES: CCc1ccc2c(-c3ncc(-c4ccccc4)cn3)c3cc(-c4ncccn4)ccc3c(-c3ncc(-c4ccccc4)cn3)c2c1 ; c1ccc(-c2cnc(-c3ccc4c(-c5ncc(-c6ccccc6)cn5)c5ccccc5c(-c5ncc(-c6ccccc6)cn5)c4c3)nc2)cc1 ; c1cnc(-c2c3ccccc3c(-c3ncccn3)c3cc(-c4ccc5ccccc5c4)ccc23)nc1 ; c1cnc(-c2c3ccccc3c(-c3ncccn3)c3cc(-c4cccc5ccccc45)ccc23)nc1]

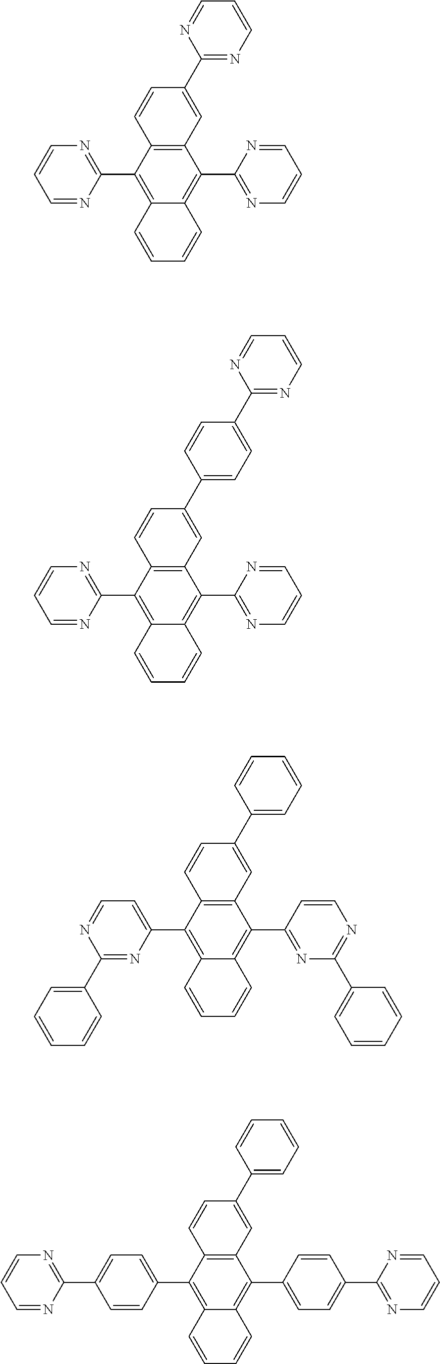 [SMILES: c1cnc(-c2ccc3c(-c4ncccn4)c4ccccc4c(-c4ncccn4)c3c2)nc1 ; c1cnc(-c2ccc(-c3ccc4c(-c5ncccn5)c5ccccc5c(-c5ncccn5)c4c3)cc2)nc1 ; c1ccc(-c2ccc3c(-c4ccnc(-c5ccccc5)n4)c4ccccc4c(-c4ccnc(-c5ccccc5)n4)c3c2)cc1 ; c1ccc(-c2ccc3c(-c4ccc(-c5ncccn5)cc4)c4ccccc4c(-c4ccc(-c5ncccn5)cc4)c3c2)cc1]

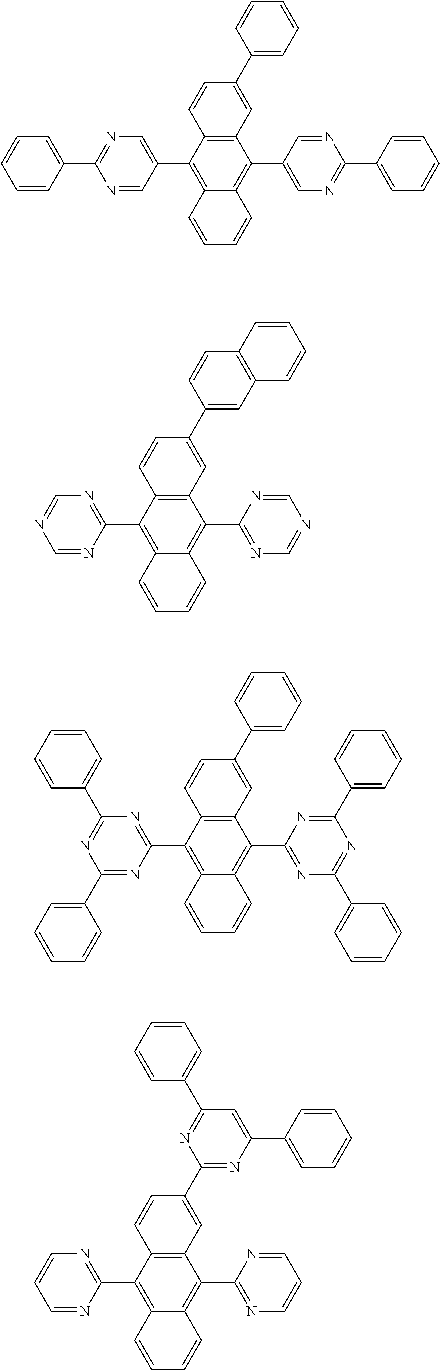 [SMILES: c1ccc(-c2ccc3c(-c4cnc(-c5ccccc5)nc4)c4ccccc4c(-c4cnc(-c5ccccc5)nc4)c3c2)cc1 ; c1ccc2cc(-c3ccc4c(-c5ncncn5)c5ccccc5c(-c5ncncn5)c4c3)ccc2c1 ; c1ccc(-c2ccc3c(-c4nc(-c5ccccc5)nc(-c5ccccc5)n4)c4ccccc4c(-c4nc(-c5ccccc5)nc(-c5ccccc5)n4)c3c2)cc1 ; c1ccc(-c2cc(-c3ccccc3)nc(-c3ccc4c(-c5ncccn5)c5ccccc5c(-c5ncccn5)c4c3)n2)cc1]

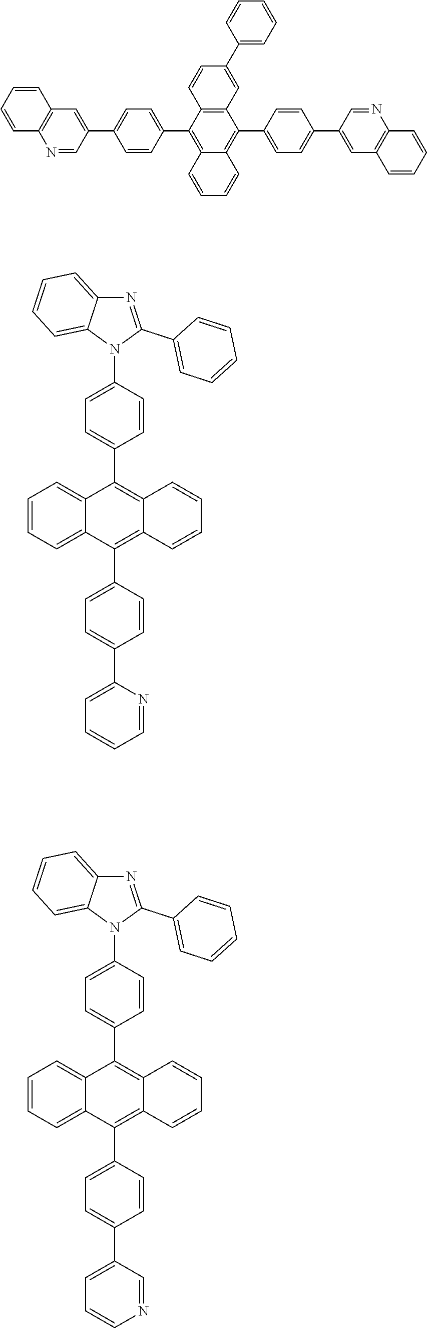 [SMILES: c1ccc(-c2ccc3c(-c4ccc(-c5cnc6ccccc6c5)cc4)c4ccccc4c(-c4ccc(-c5cnc6ccccc6c5)cc4)c3c2)cc1 ; c1ccc(-c2nc3ccccc3n2-c2ccc(-c3c4ccccc4c(-c4ccc(-c5ccccn5)cc4)c4ccccc34)cc2)cc1 ; c1ccc(-c2nc3ccccc3n2-c2ccc(-c3c4ccccc4c(-c4ccc(-c5cccnc5)cc4)c4ccccc34)cc2)cc1]

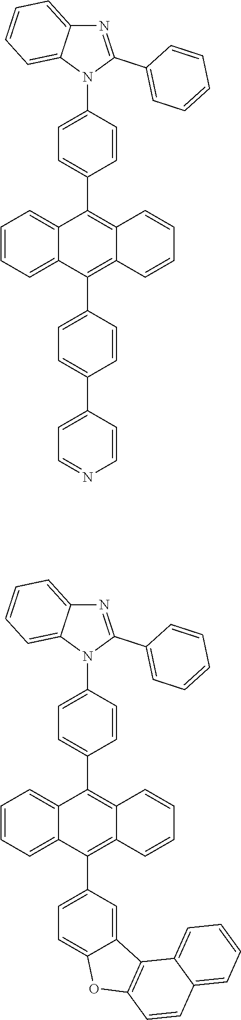 [SMILES: c1ccc(-c2nc3ccccc3n2-c2ccc(-c3c4ccccc4c(-c4ccc(-c5ccncc5)cc4)c4ccccc34)cc2)cc1 ; c1ccc(-c2nc3ccccc3n2-c2ccc(-c3c4ccccc4c(-c4ccc5oc6ccc7ccccc7c6c5c4)c4ccccc34)cc2)cc1]

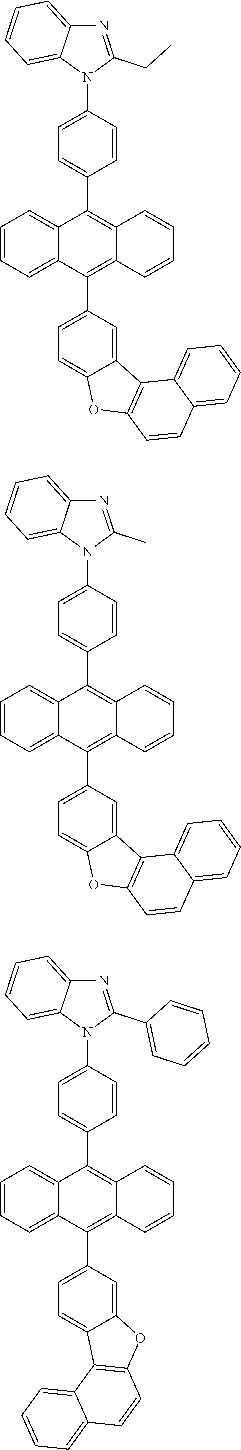 [SMILES: CCc1nc2ccccc2n1-c1ccc(-c2c3ccccc3c(-c3ccc4oc5ccc6ccccc6c5c4c3)c3ccccc23)cc1 ; Cc1nc2ccccc2n1-c1ccc(-c2c3ccccc3c(-c3ccc4oc5ccc6ccccc6c5c4c3)c3ccccc23)cc1 ; c1ccc(-c2nc3ccccc3n2-c2ccc(-c3c4ccccc4c(-c4ccc5c(c4)oc4ccc6ccccc6c45)c4ccccc34)cc2)cc1]

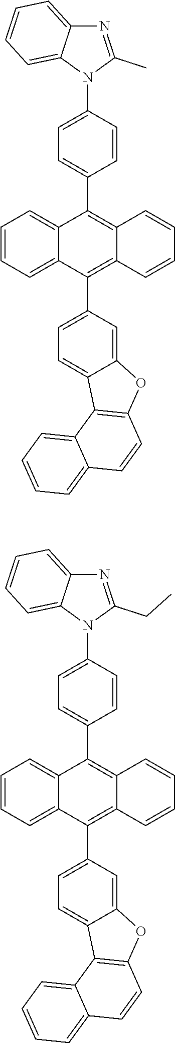 [SMILES: Cc1nc2ccccc2n1-c1ccc(-c2c3ccccc3c(-c3ccc4c(c3)oc3ccc5ccccc5c34)c3ccccc23)cc1 ; CCc1nc2ccccc2n1-c1ccc(-c2c3ccccc3c(-c3ccc4c(c3)oc3ccc5ccccc5c34)c3ccccc23)cc1]

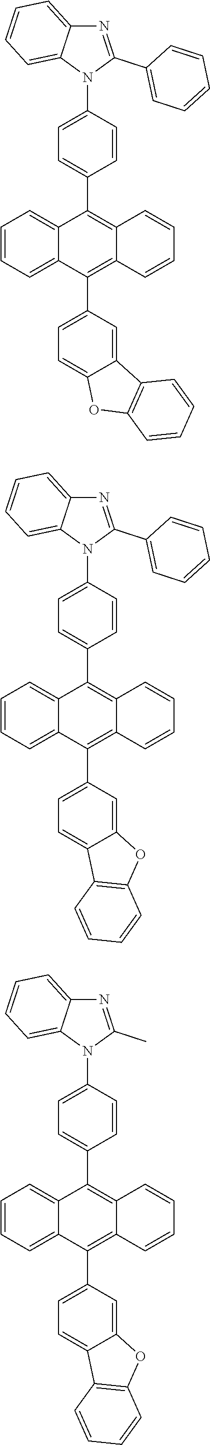 [SMILES: c1ccc(-c2nc3ccccc3n2-c2ccc(-c3c4ccccc4c(-c4ccc5oc6ccccc6c5c4)c4ccccc34)cc2)cc1 ; c1ccc(-c2nc3ccccc3n2-c2ccc(-c3c4ccccc4c(-c4ccc5c(c4)oc4ccccc45)c4ccccc34)cc2)cc1 ; Cc1nc2ccccc2n1-c1ccc(-c2c3ccccc3c(-c3ccc4c(c3)oc3ccccc34)c3ccccc23)cc1]

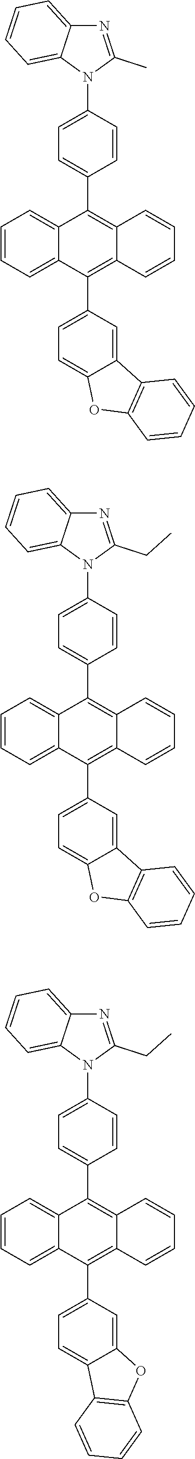 [SMILES: Cc1nc2ccccc2n1-c1ccc(-c2c3ccccc3c(-c3ccc4oc5ccccc5c4c3)c3ccccc23)cc1 ; CCc1nc2ccccc2n1-c1ccc(-c2c3ccccc3c(-c3ccc4oc5ccccc5c4c3)c3ccccc23)cc1 ; CCc1nc2ccccc2n1-c1ccc(-c2c3ccccc3c(-c3ccc4c(c3)oc3ccccc34)c3ccccc23)cc1]

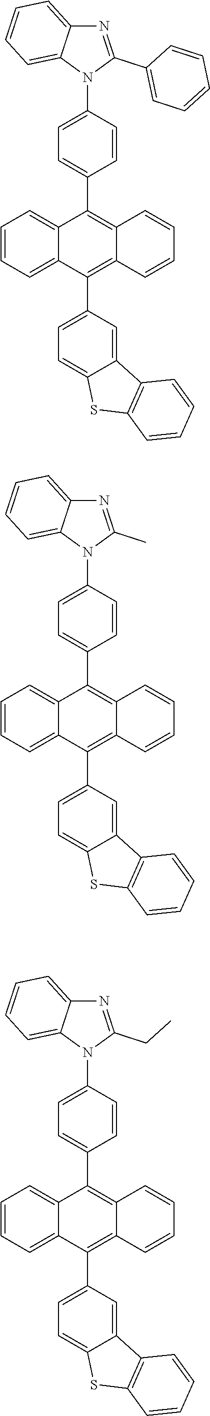 [SMILES: c1ccc(-c2nc3ccccc3n2-c2ccc(-c3c4ccccc4c(-c4ccc5sc6ccccc6c5c4)c4ccccc34)cc2)cc1 ; Cc1nc2ccccc2n1-c1ccc(-c2c3ccccc3c(-c3ccc4sc5ccccc5c4c3)c3ccccc23)cc1 ; CCc1nc2ccccc2n1-c1ccc(-c2c3ccccc3c(-c3ccc4sc5ccccc5c4c3)c3ccccc23)cc1]

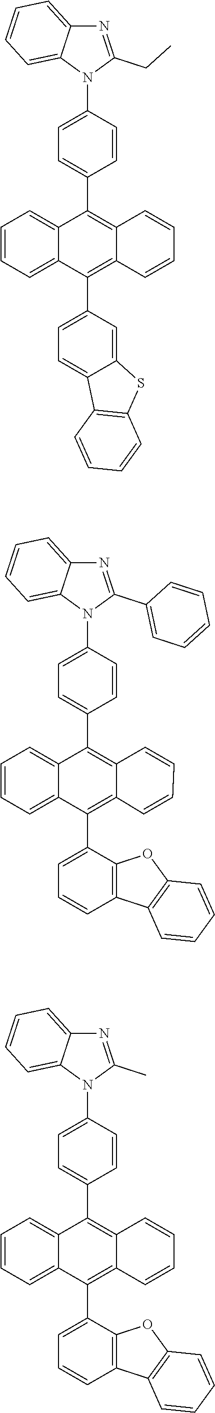 [SMILES: CCc1nc2ccccc2n1-c1ccc(-c2c3ccccc3c(-c3ccc4c(c3)sc3ccccc34)c3ccccc23)cc1 ; c1ccc(-c2nc3ccccc3n2-c2ccc(-c3c4ccccc4c(-c4cccc5c4oc4ccccc45)c4ccccc34)cc2)cc1 ; Cc1nc2ccccc2n1-c1ccc(-c2c3ccccc3c(-c3cccc4c3oc3ccccc34)c3ccccc23)cc1]

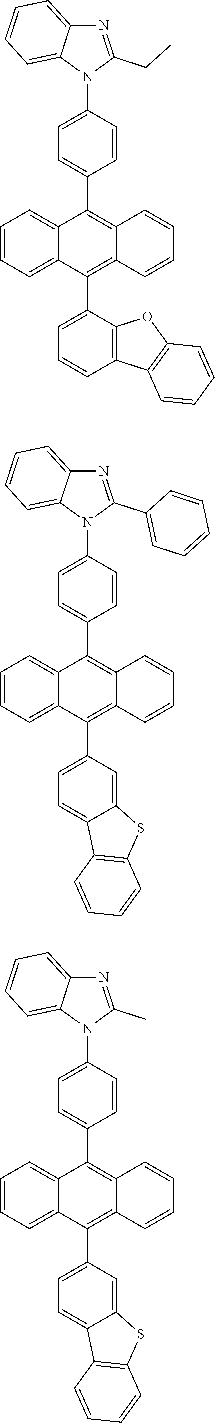 [SMILES: CCc1nc2ccccc2n1-c1ccc(-c2c3ccccc3c(-c3cccc4c3oc3ccccc34)c3ccccc23)cc1 ; c1ccc(-c2nc3ccccc3n2-c2ccc(-c3c4ccccc4c(-c4ccc5c(c4)sc4ccccc45)c4ccccc34)cc2)cc1 ; Cc1nc2ccccc2n1-c1ccc(-c2c3ccccc3c(-c3ccc4c(c3)sc3ccccc34)c3ccccc23)cc1]

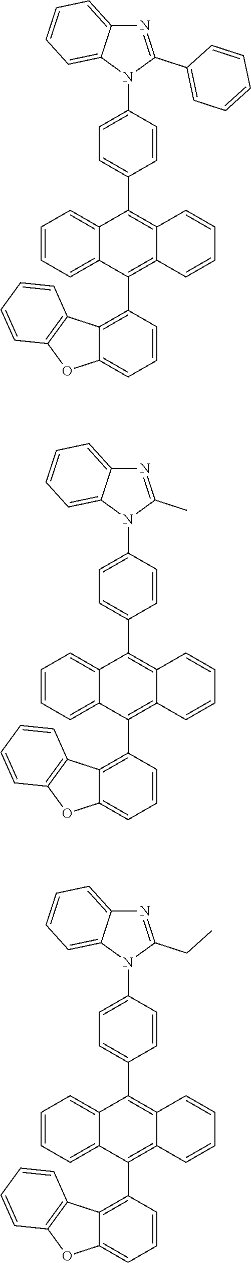 [SMILES: c1ccc(-c2nc3ccccc3n2-c2ccc(-c3c4ccccc4c(-c4cccc5oc6ccccc6c45)c4ccccc34)cc2)cc1 ; Cc1nc2ccccc2n1-c1ccc(-c2c3ccccc3c(-c3cccc4oc5ccccc5c34)c3ccccc23)cc1 ; CCc1nc2ccccc2n1-c1ccc(-c2c3ccccc3c(-c3cccc4oc5ccccc5c34)c3ccccc23)cc1]

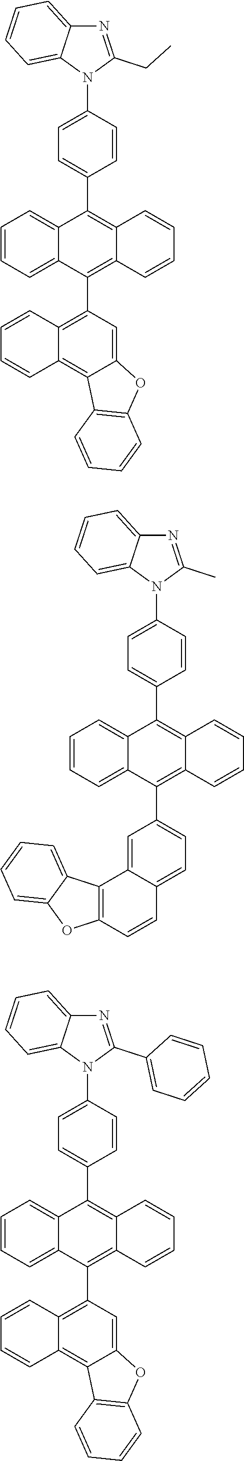

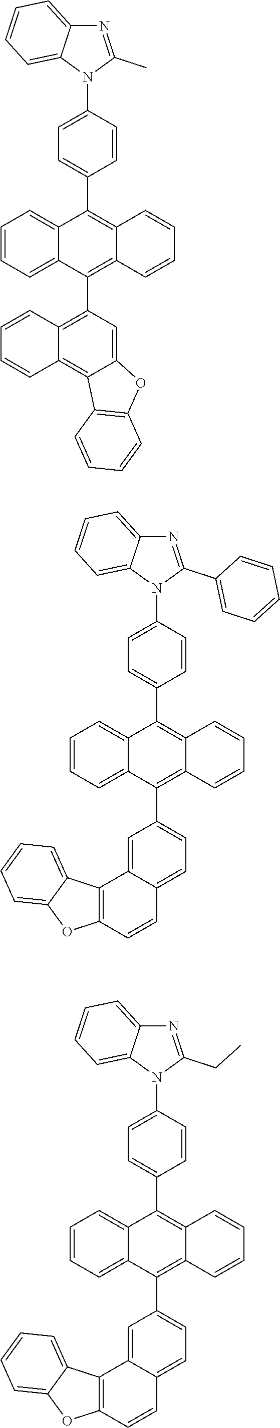

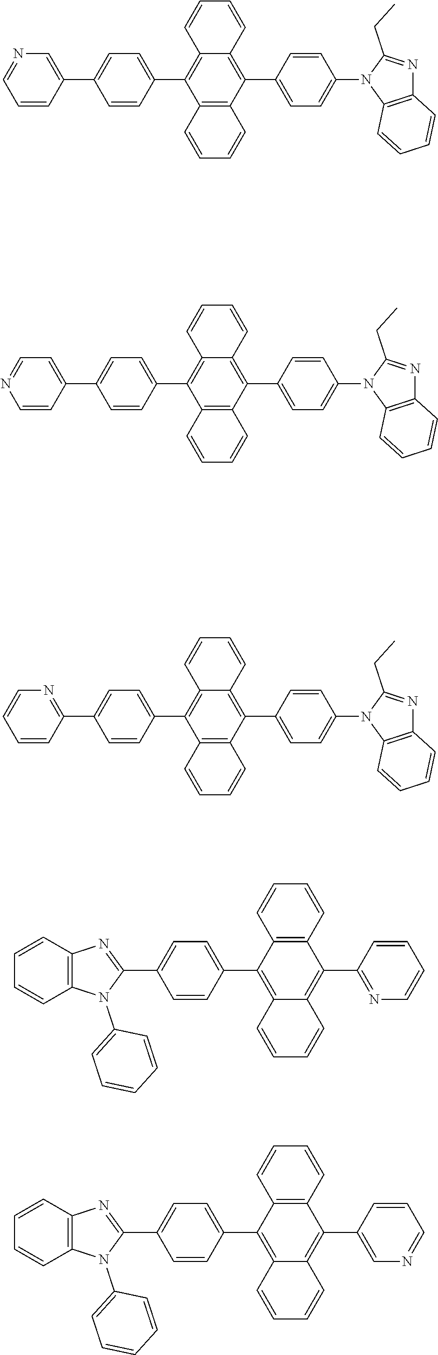 [SMILES: CCc1nc2ccccc2n1-c1ccc(-c2c3ccccc3c(-c3ccc(-c4cccnc4)cc3)c3ccccc23)cc1 ; CCc1nc2ccccc2n1-c1ccc(-c2c3ccccc3c(-c3ccc(-c4ccncc4)cc3)c3ccccc23)cc1 ; CCc1nc2ccccc2n1-c1ccc(-c2c3ccccc3c(-c3ccc(-c4ccccn4)cc3)c3ccccc23)cc1 ; c1ccc(-n2c(-c3ccc(-c4c5ccccc5c(-c5ccccn5)c5ccccc45)cc3)nc3ccccc32)cc1 ; c1ccc(-n2c(-c3ccc(-c4c5ccccc5c(-c5cccnc5)c5ccccc45)cc3)nc3ccccc32)cc1]

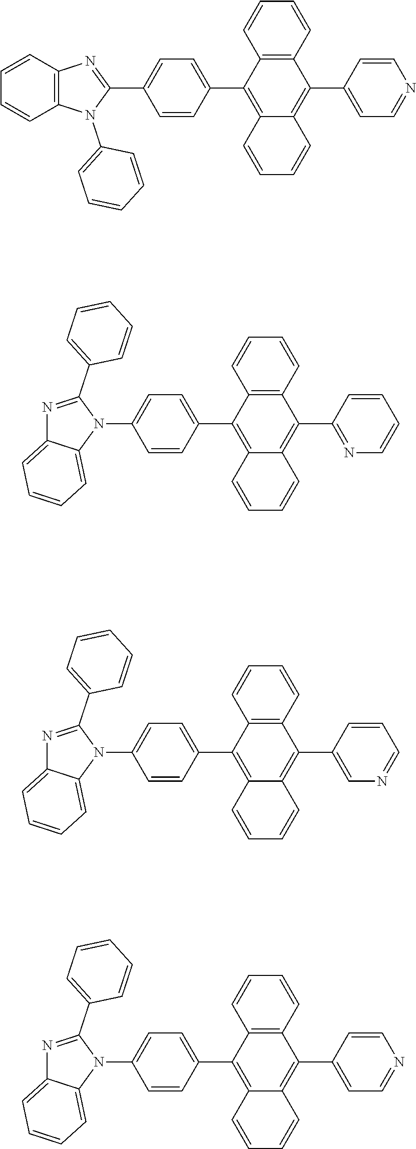 [SMILES: c1ccc(-n2c(-c3ccc(-c4c5ccccc5c(-c5ccncc5)c5ccccc45)cc3)nc3ccccc32)cc1 ; c1ccc(-c2nc3ccccc3n2-c2ccc(-c3c4ccccc4c(-c4ccccn4)c4ccccc34)cc2)cc1 ; c1ccc(-c2nc3ccccc3n2-c2ccc(-c3c4ccccc4c(-c4cccnc4)c4ccccc34)cc2)cc1 ; c1ccc(-c2nc3ccccc3n2-c2ccc(-c3c4ccccc4c(-c4ccncc4)c4ccccc34)cc2)cc1]

### [Compound Represented by Formula (3)]

A compound represented by the formula (3) is shown as follows:

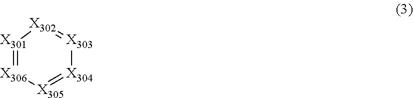

wherein in the formula (3),
X₃₀₁ to X₃₀₆ are independently N, CR₃₀₁ or CR₃₀₂; provided that at least two of X₃₀₁ to X₃₀₆ are N, at least one thereof is CR₃₀₁, and at least two thereof are CR₃₀₂; when two or more R₃₀₁'s are present, the two or more R₃₀₁'s may be the same as or different from each other; two or more R₃₀₂'s may be the same as or different from each other;
R₃₀₁ is a monovalent group represented by the following formula (3A):

(Ar₃₀₁)_{m3A}-(L₃₀₁)_{n3A}-* (3A)

wherein in the formula (3A),
L₃₀₁ is
a single bond,
a substituted or unsubstituted aromatic hydrocarbon ring group having 6 to 50 ring carbon atoms, or
a substituted or unsubstituted heterocyclic group having 5 to 50 ring atoms;
Ar₃₀₁ is
a substituted or unsubstituted dibenzofuranyl group,
a substituted or unsubstituted dibenzothiophenyl group,
a substituted or unsubstituted carbazolyl group,
a substituted or unsubstituted phenanthryl group,
a substituted or unsubstituted monovalent group including a nitrogen-containing five-membered ring, or
a monovalent group represented by the following formula (3B):

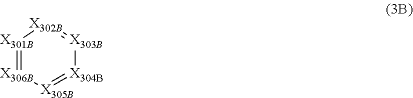

wherein in the formula (3B),
X_{301B} to X_{306B} are independently N, CR_{301B} or CR_{302B}; provided that one of X_{301B} to X_{306B} is CR_{301B}, and at least one thereof is N;
R_{301B} is a single bond bonding with L₃₀₁;
when two or more R_{302B}'s are present, the adjacent two of R_{302B}'S form a substituted or unsubstituted, saturated or unsaturated fused ring by bonding with each other, or do not form the substituted or unsubstituted, saturated or unsaturated fused ring;
R_{302B} which do not form the substituted or unsubstituted, saturated or unsaturated fused ring is a hydrogen atom, or a substituent R;
when two or more R_{302B}'s are present, the two or more R_{302B}'s may be the same as or different from each other;
m3A is an integer of 1 to 5; when m3A is 2 or more, two or more Ar₃₀₁'s may be the same as or different from each other;
n3A is an integer of 1 to 3; when n3A is 2 or more, two or more L₃₀₁'s may be the same as or different from each other; provided that when all of L₃₀₁'s are a single bond, m3A is 1;
R₃₀₂ is a hydrogen atom, or a substituent R; and
the substituent R is the same as defined in the formula (1).

When m3A is 2 or more in the formula (3A), each of two or more Ar₃₀₁'s is directly bonded with L₃₀₁. Further, when a plurality of L₃₀₁'s is present (when n3A is 2 or more), Ar₃₀₁ is directly bonded with leftmost L₃₀₁ on paper. When m3A is 2 or more, each of two or more Ar₃₀₁'s is directly bonded with leftmost L₃₀₁ on paper. For example, when m3A is 2 and nA3 is 2, the group represented by the formula (3A) is the following structure.

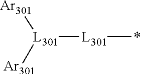

As understood from the above-described definition, since R₃₀₁ is included in the definition of R₃₀₂, R₃₀₁ can be also referred to as the specific aspect of R₃₀₂. That is, in at least two R₃₀₂'s of CR₃₀₂ for X₃₀₁ to X₃₀₆ in the formula (3), both of them may be R₃₀₁, one of them may be R₃₀₁ and the other may be a hydrogen atom or a substituent R other than R₃₀₁, or both of them may be a substituent R other than R₃₀₁.

In one embodiment, R₃₀₂ is a hydrogen atom, or a substituent R′.

The substituent R′ is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,
—Si(R₄₁)(R₄₂)(R₄₃),
—C(═O)R₄₄, —COOR₄₅,
—S(═O)₂R₄₆,
—P(═O)(R₄₇)(R₄₈),
—Ge(R₄₉)(R₅₀)(R₅₁),
—N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other),
a hydroxy group,
a halogen atom,
a cyano group,
a nitro group,
an aryl group having 6 to 50 ring carbon atoms, and a monovalent heterocyclic group having 5 to 50 ring atoms.

In one embodiment, two of X₃₀₁ to X₃₀₆ are N, two thereof are CR₃₀₁, and two thereof are CR₃₀₂.

In one embodiment, the compound represented by the formula (3) is a compound represented by the following formula (3-1):

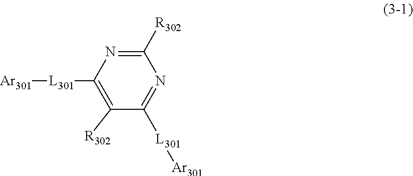

wherein in the formula (3-1),
R₃₀₂, L₃₀₁, and Ar₃₀₁ are the same as defined in the formula (3).

In one embodiment, the compound represented by the formula (3) is a compound represented by the following formula (3-11):

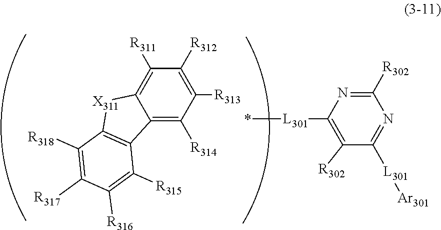

wherein in the formula (3-11),
R₃₀₂, L₃₀₁, and Ar₃₀₁ are the same as defined in the formula (3);
X₃₁₁ is O, S or NR₃₁₉;
one of R₃₁₁ to R₃₁₉ is bonded with L₃₀₁ via a single bond;
R₃₁₁ to R₃₁₉ which are not bonded with L₃₀₁ are independently a hydrogen atom, or a substituent R; and
the substituent R is the same as defined in the formula (1).

In one embodiment, three of X₃₀₁ to X₃₀₆ are N, two thereof are CR₃₀₁, and one thereof are CR₃₀₂.

In one embodiment, the compound represented by the formula (3) is a compound represented by the following formula (3-2):

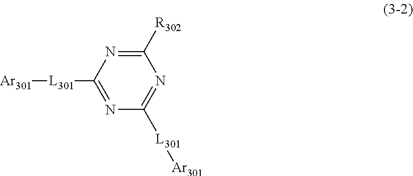

wherein in the formula (3-2),
R₃₀₂, L₃₀₁, and Ar₃₀₁ are the same as defined in the formula (3).

In one embodiment, the compound represented by the formula (3) is a compound represented by the following formula (3-21):

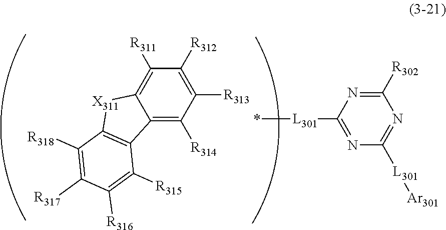

wherein in the formula (3-21),
R₃₀₂, L₃₀₁, and Ar₃₀₁ are the same as defined in the formula (3);
X₃₁₁ is O, S or NR₃₁₉;
one of R₃₁₁ to R₃₁₉ is bonded with L₃₀₁ via a single bond;
R₃₁₁ to R₃₁₉ which are not bonded with L₃₀₁ are independently a hydrogen atom, or a substituent R; and
the substituent R is the same as defined in the formula (1).

In one embodiment, at least two of X₃₀₁ to X₃₀₆ in the formula (3) are N, one thereof is CR₃₀₁, and at least two thereof are CR₃₀₂.

In one embodiment, the compound represented by the formula (3) is a compound represented by the following formula (3-3):

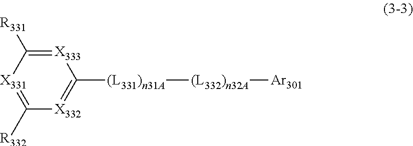

wherein in the formula (3-3), Ar₃₀₁ is the same as defined in the formula (3);
X₃₃₁ to X₃₃₃ are independently N or CR₃₃₃; provided that at least two of X₃₃₁ to X₃₃₃ are N;
R₃₃₁ and R₃₃₂ are independently
a substituted or unsubstituted alkyl group having 1 to 25 carbon atoms,
a substituted or unsubstituted cycloalkyl group having 3 to 18 ring carbon atoms,
a cyano group,
a substituted or unsubstituted aryl group having 6 to 30 ring carbon atoms, or
a substituted or unsubstituted monovalent heterocyclic group having 5 to 30 ring atoms;
R₃₃₃ is
a hydrogen atom,
a substituted or unsubstituted alkyl group having 1 to 25 carbon atoms,
a substituted or unsubstituted cycloalkyl group having 3 to 18 ring carbon atoms,
a cyano group,
a substituted or unsubstituted aryl group having 6 to 30 ring carbon atoms, or
a substituted or unsubstituted monovalent heterocyclic group having 5 to 30 ring atoms;
L₃₃₁ is
a substituted or unsubstituted aromatic hydrocarbon ring group having 6 to 30 ring carbon atoms, or
a substituted or unsubstituted heterocyclic group having 5 to 30 ring atoms;
L₃₃₂ is
a substituted or unsubstituted aromatic hydrocarbon ring group having 6 to 30 ring carbon atoms, or
a substituted or unsubstituted heterocyclic group containing at least one nitrogen atom and having 5 to 30 ring atoms;
n31A is an integer of 0 to 2; when n31A is 2, two L₃₃₁'s may be the same as or different from each other;
n32A is an integer of 0 to 2; when n32A is 2, two L₃₃₂'s may be the same as or different from each other; and
provided that the sum of n31A and n32A is two or more.

In one embodiment, the compound represented by the formula (3-3) is a compound represented by the following formula (3-31):

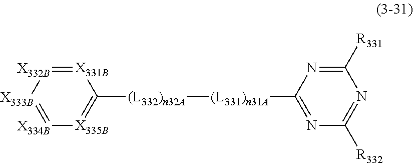

wherein in the formula (3-31),
R₃₃₁, R₃₃₂, L₃₃₁, L₃₃₂, n31A, and n32A are the same as defined in the formula (3-3);
X_{331B} to X_{335B} are independently N or CR_{331B}; provided that at least one of X_{331B} to X_{335B} are N;
when two or more R_{331B}'s are present, the adjacent two of R_{331B}'s form a substituted or unsubstituted, saturated or unsaturated fused ring by bonding with each other, or do not form the substituted or unsubstituted, saturated or unsaturated fused ring;
R_{331B} which do not form the substituted or unsubstituted, saturated or unsaturated fused ring is a hydrogen atom, or a substituent R;
when two or more R_{331B}'s are present, the two or more R_{331B}'s may be the same as or different from each other; and
the substituent R is the same as defined in the formula (1).

In one embodiment, a substituent in the case of “substituted or unsubstituted” in the formula (3) is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,
—Si(R₄₁)(R₄₂)(R₄₃),
—C(═O)R₄₄, —COOR₄₅,
—S(═O)₂R₄₆,
—P(═O)(R₄₇)(R₄₃),
—Ge(R₄₉)(R₅₀)(R₅₁),
—N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other),
a hydroxy group,
a halogen atom,
a cyano group,
a nitro group,
an aryl group having 6 to 50 ring carbon atoms, and
a monovalent heterocyclic group having 5 to 50 ring atoms.

In one embodiment, a substituent in the case of “substituted or unsubstituted” in the formula (3) is selected from the group consisting of
an alkyl group having 1 to 18 carbon atoms,
an aryl group having 6 to 18 ring carbon atoms, and
a monovalent heterocyclic group having 5 to 18 ring atoms.

The compound represented by the formula (3) can be synthesized by using known alternative reactions or raw materials adapted to the target compound.

Specific examples of the compound represented by the formula (3) will be described below, but these are merely examples, and the compound represented by the formula (3) is not limited to the following specific examples.

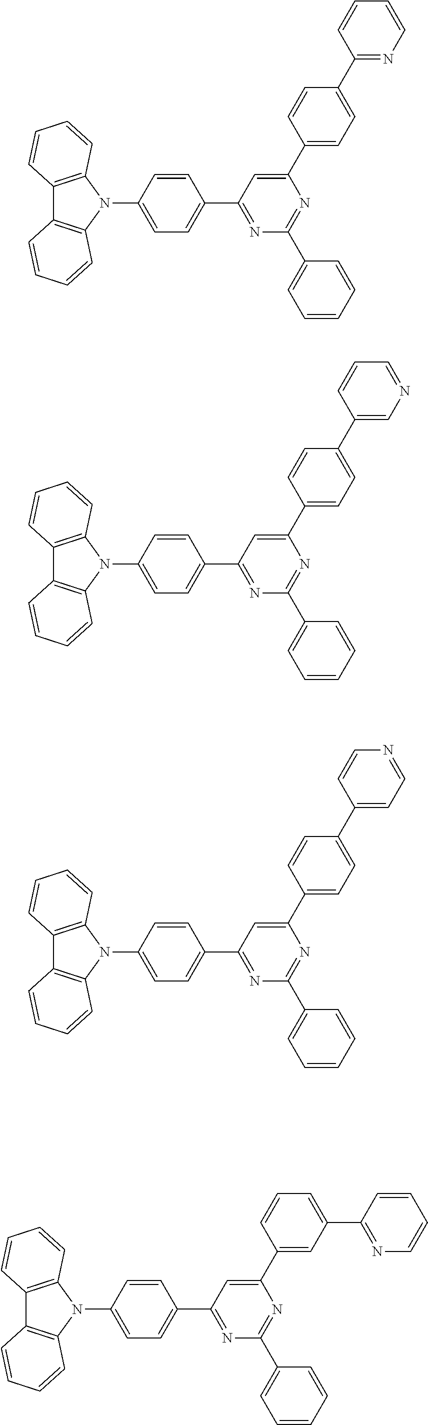

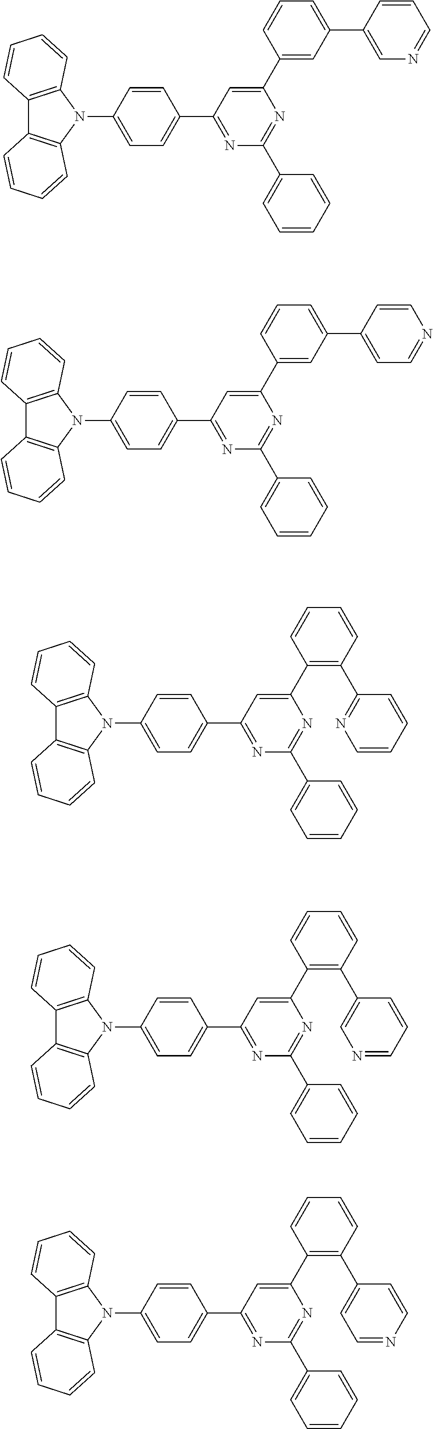 [SMILES: c1ccc(-c2nc(-c3ccc(-n4c5ccccc5c5ccccc54)cc3)cc(-c3cccc(-c4cccnc4)c3)n2)cc1 ; c1ccc(-c2nc(-c3ccc(-n4c5ccccc5c5ccccc54)cc3)cc(-c3cccc(-c4ccncc4)c3)n2)cc1 ; c1ccc(-c2nc(-c3ccc(-n4c5ccccc5c5ccccc54)cc3)cc(-c3ccccc3-c3ccccn3)n2)cc1 ; c1ccc(-c2nc(-c3ccc(-n4c5ccccc5c5ccccc54)cc3)cc(-c3ccccc3-c3cccnc3)n2)cc1 ; c1ccc(-c2nc(-c3ccc(-n4c5ccccc5c5ccccc54)cc3)cc(-c3ccccc3-c3ccncc3)n2)cc1]

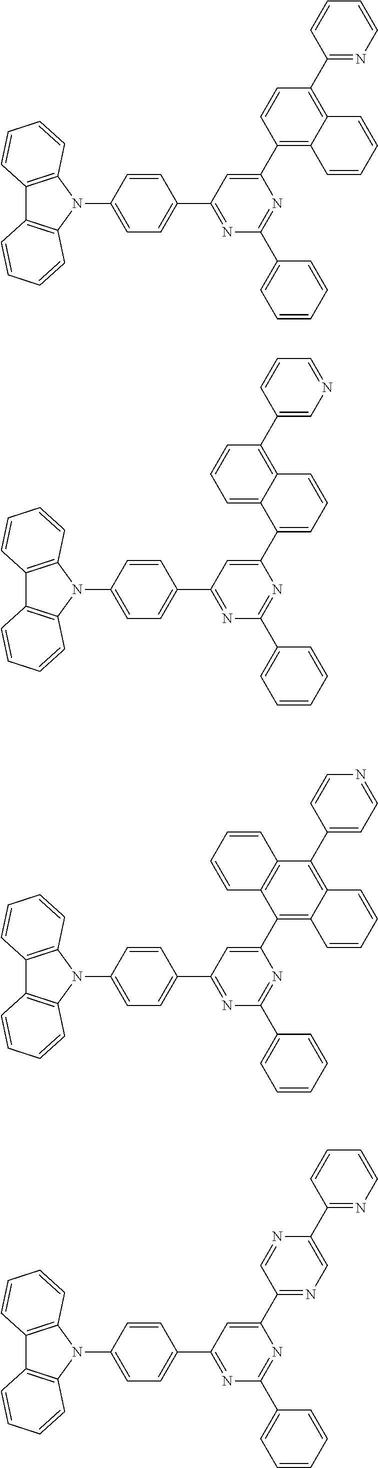

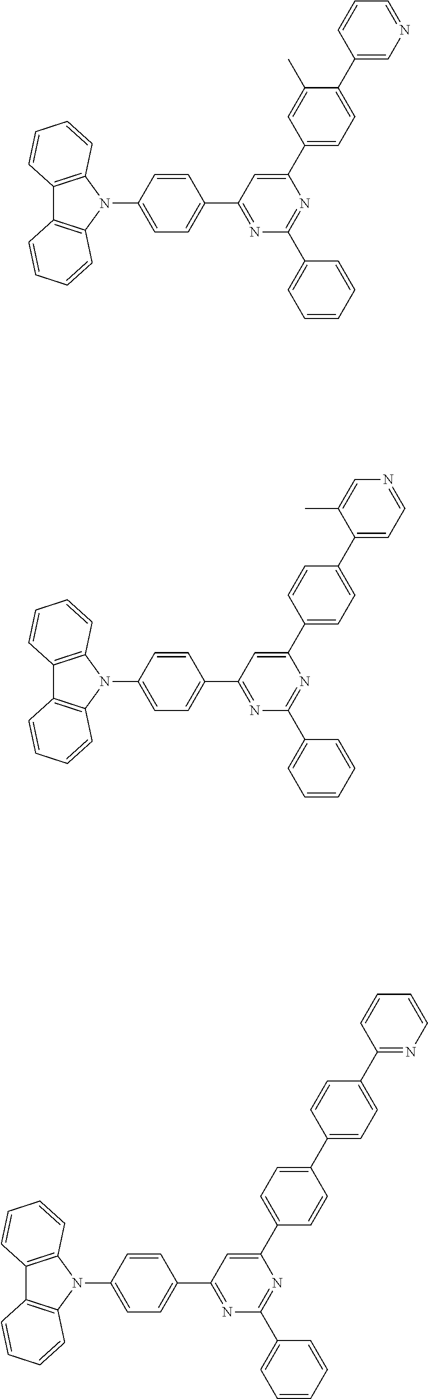 [SMILES: Cc1cc(-c2cc(-c3ccc(-n4c5ccccc5c5ccccc54)cc3)nc(-c3ccccc3)n2)ccc1-c1cccnc1 ; Cc1cnccc1-c1ccc(-c2cc(-c3ccc(-n4c5ccccc5c5ccccc54)cc3)nc(-c3ccccc3)n2)cc1 ; c1ccc(-c2nc(-c3ccc(-c4ccc(-c5ccccn5)cc4)cc3)cc(-c3ccc(-n4c5ccccc5c5ccccc54)cc3)n2)cc1]

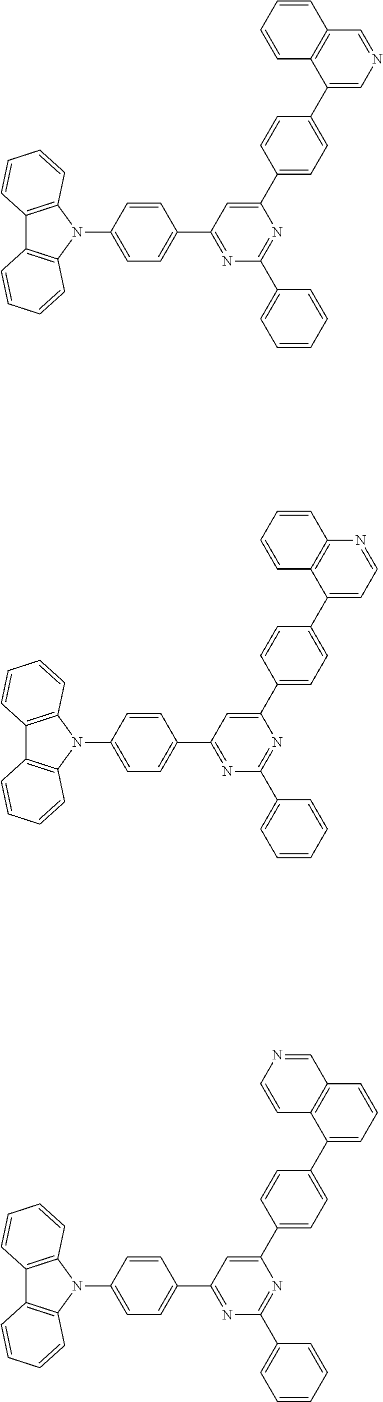 [SMILES: c1ccc(-c2nc(-c3ccc(-c4cncc5ccccc45)cc3)cc(-c3ccc(-n4c5ccccc5c5ccccc54)cc3)n2)cc1 ; c1ccc(-c2nc(-c3ccc(-c4ccnc5ccccc45)cc3)cc(-c3ccc(-n4c5ccccc5c5ccccc54)cc3)n2)cc1 ; c1ccc(-c2nc(-c3ccc(-c4cccc5cnccc45)cc3)cc(-c3ccc(-n4c5ccccc5c5ccccc54)cc3)n2)cc1]

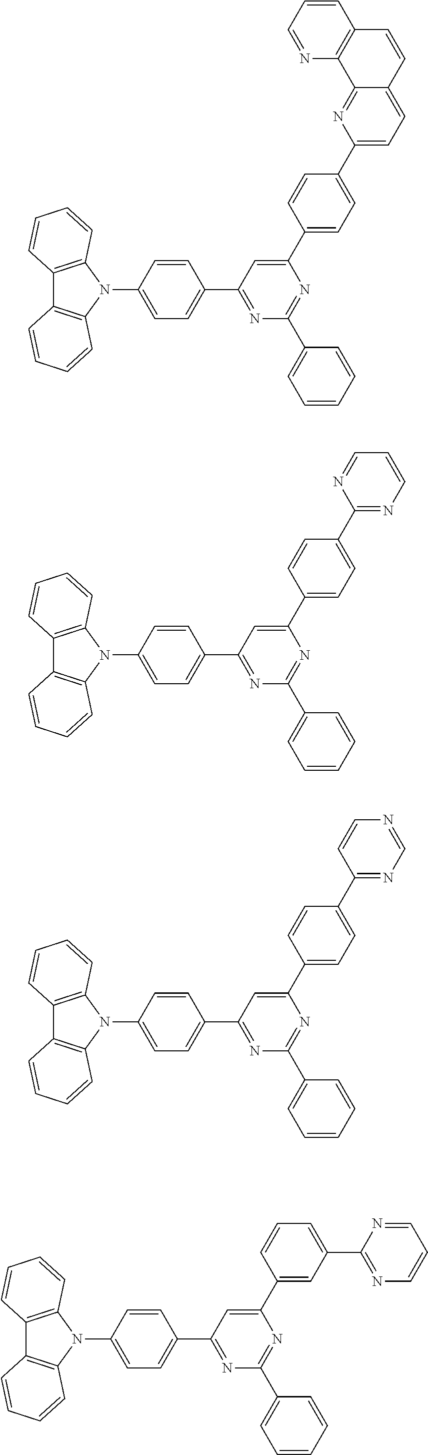 [SMILES: c1ccc(-c2nc(-c3ccc(-c4ccc5ccc6cccnc6c5n4)cc3)cc(-c3ccc(-n4c5ccccc5c5ccccc54)cc3)n2)cc1 ; c1ccc(-c2nc(-c3ccc(-c4ncccn4)cc3)cc(-c3ccc(-n4c5ccccc5c5ccccc54)cc3)n2)cc1 ; c1ccc(-c2nc(-c3ccc(-c4ccncn4)cc3)cc(-c3ccc(-n4c5ccccc5c5ccccc54)cc3)n2)cc1 ; c1ccc(-c2nc(-c3ccc(-n4c5ccccc5c5ccccc54)cc3)cc(-c3cccc(-c4ncccn4)c3)n2)cc1]

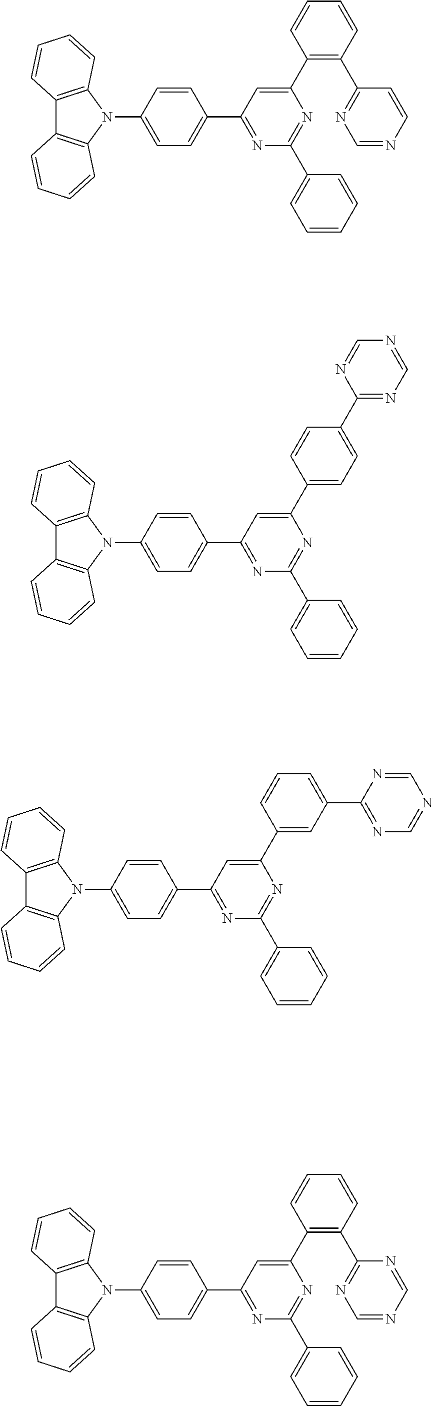 [SMILES: c1ccc(-c2nc(-c3ccc(-n4c5ccccc5c5ccccc54)cc3)cc(-c3ccccc3-c3ccncn3)n2)cc1 ; c1ccc(-c2nc(-c3ccc(-c4ncncn4)cc3)cc(-c3ccc(-n4c5ccccc5c5ccccc54)cc3)n2)cc1 ; c1ccc(-c2nc(-c3ccc(-n4c5ccccc5c5ccccc54)cc3)cc(-c3cccc(-c4ncncn4)c3)n2)cc1 ; c1ccc(-c2nc(-c3ccc(-n4c5ccccc5c5ccccc54)cc3)cc(-c3ccccc3-c3ncncn3)n2)cc1]

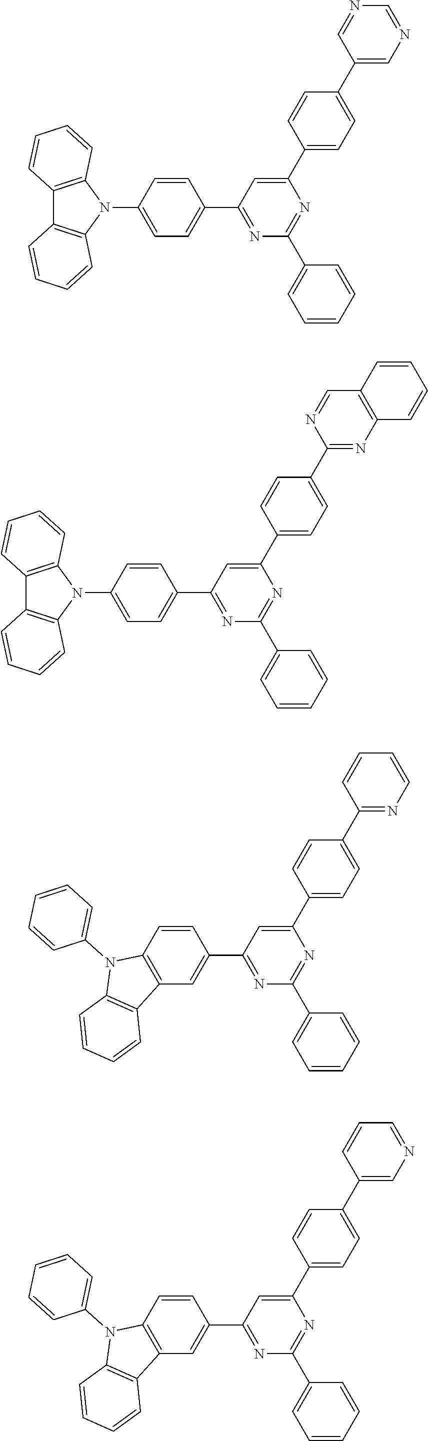 [SMILES: c1ccc(-c2nc(-c3ccc(-c4cncnc4)cc3)cc(-c3ccc(-n4c5ccccc5c5ccccc54)cc3)n2)cc1 ; c1ccc(-c2nc(-c3ccc(-c4ncc5ccccc5n4)cc3)cc(-c3ccc(-n4c5ccccc5c5ccccc54)cc3)n2)cc1 ; c1ccc(-c2nc(-c3ccc(-c4ccccn4)cc3)cc(-c3ccc4c(c3)c3ccccc3n4-c3ccccc3)n2)cc1 ; c1ccc(-c2nc(-c3ccc(-c4cccnc4)cc3)cc(-c3ccc4c(c3)c3ccccc3n4-c3ccccc3)n2)cc1]

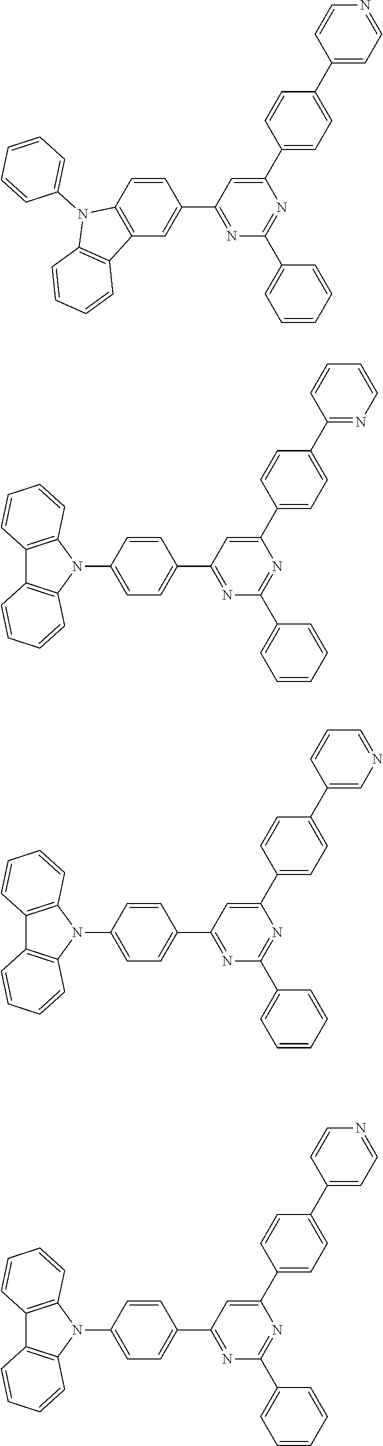

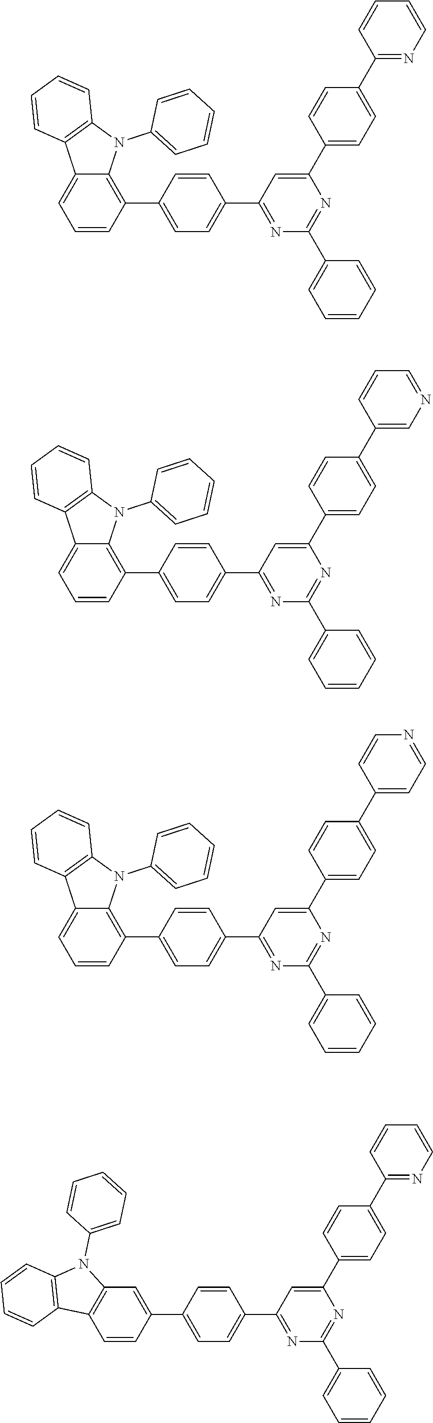 [SMILES: c1ccc(-c2nc(-c3ccc(-c4ccccn4)cc3)cc(-c3ccc(-c4cccc5c6ccccc6n(-c6ccccc6)c45)cc3)n2)cc1 ; c1ccc(-c2nc(-c3ccc(-c4cccnc4)cc3)cc(-c3ccc(-c4cccc5c6ccccc6n(-c6ccccc6)c45)cc3)n2)cc1 ; c1ccc(-c2nc(-c3ccc(-c4ccncc4)cc3)cc(-c3ccc(-c4cccc5c6ccccc6n(-c6ccccc6)c45)cc3)n2)cc1 ; c1ccc(-c2nc(-c3ccc(-c4ccc5c6ccccc6n(-c6ccccc6)c5c4)cc3)cc(-c3ccc(-c4ccccn4)cc3)n2)cc1]

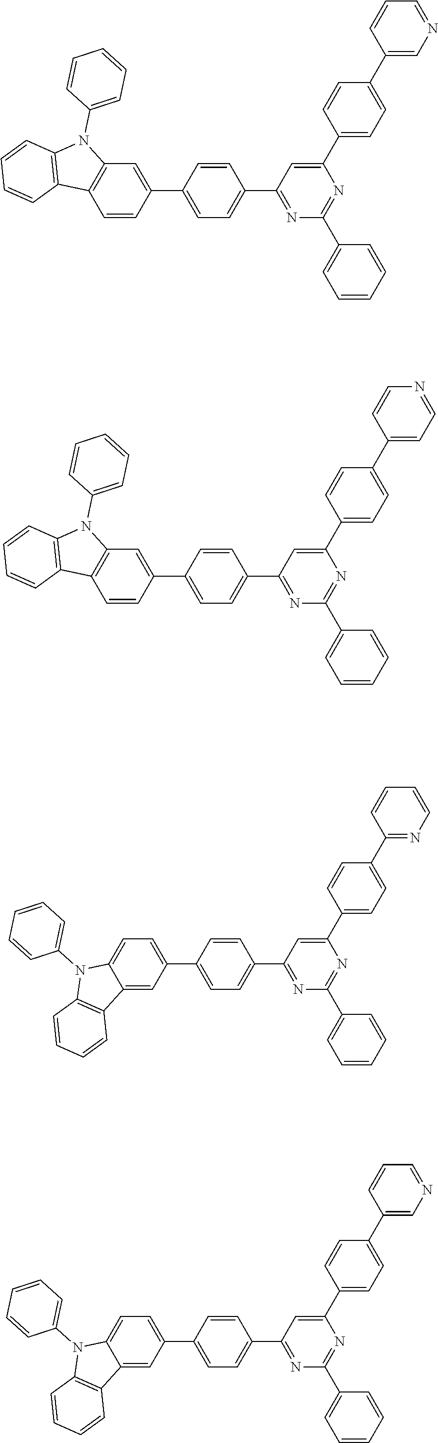 [SMILES: c1ccc(-c2nc(-c3ccc(-c4cccnc4)cc3)cc(-c3ccc(-c4ccc5c6ccccc6n(-c6ccccc6)c5c4)cc3)n2)cc1 ; c1ccc(-c2nc(-c3ccc(-c4ccncc4)cc3)cc(-c3ccc(-c4ccc5c6ccccc6n(-c6ccccc6)c5c4)cc3)n2)cc1 ; c1ccc(-c2nc(-c3ccc(-c4ccc5c(c4)c4ccccc4n5-c4ccccc4)cc3)cc(-c3ccc(-c4ccccn4)cc3)n2)cc1 ; c1ccc(-c2nc(-c3ccc(-c4cccnc4)cc3)cc(-c3ccc(-c4ccc5c(c4)c4ccccc4n5-c4ccccc4)cc3)n2)cc1]

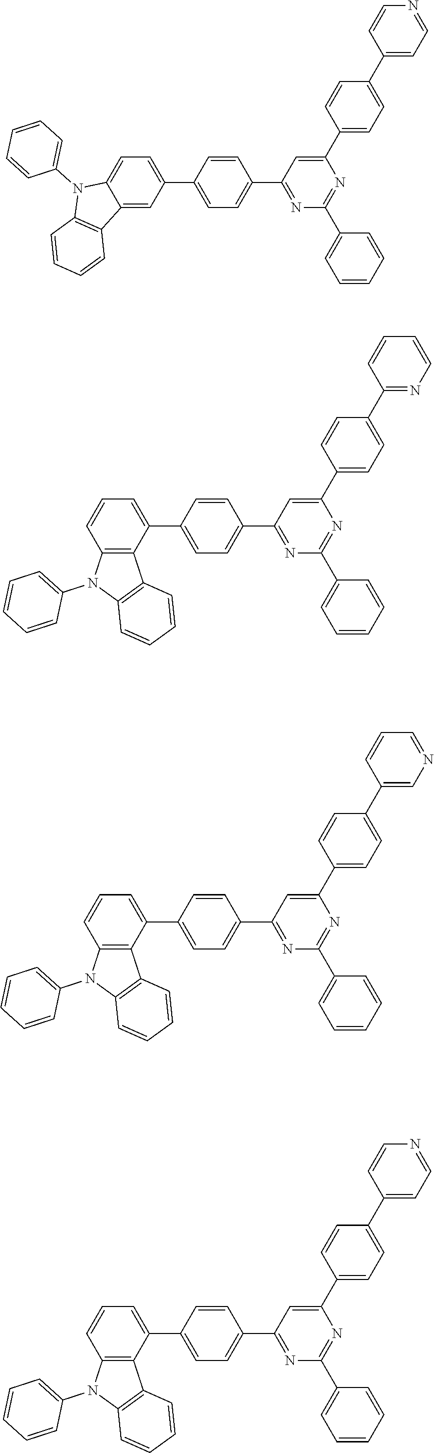 [SMILES: c1ccc(-c2nc(-c3ccc(-c4ccncc4)cc3)cc(-c3ccc(-c4ccc5c(c4)c4ccccc4n5-c4ccccc4)cc3)n2)cc1 ; c1ccc(-c2nc(-c3ccc(-c4ccccn4)cc3)cc(-c3ccc(-c4cccc5c4c4ccccc4n5-c4ccccc4)cc3)n2)cc1 ; c1ccc(-c2nc(-c3ccc(-c4cccnc4)cc3)cc(-c3ccc(-c4cccc5c4c4ccccc4n5-c4ccccc4)cc3)n2)cc1 ; c1ccc(-c2nc(-c3ccc(-c4ccncc4)cc3)cc(-c3ccc(-c4cccc5c4c4ccccc4n5-c4ccccc4)cc3)n2)cc1]

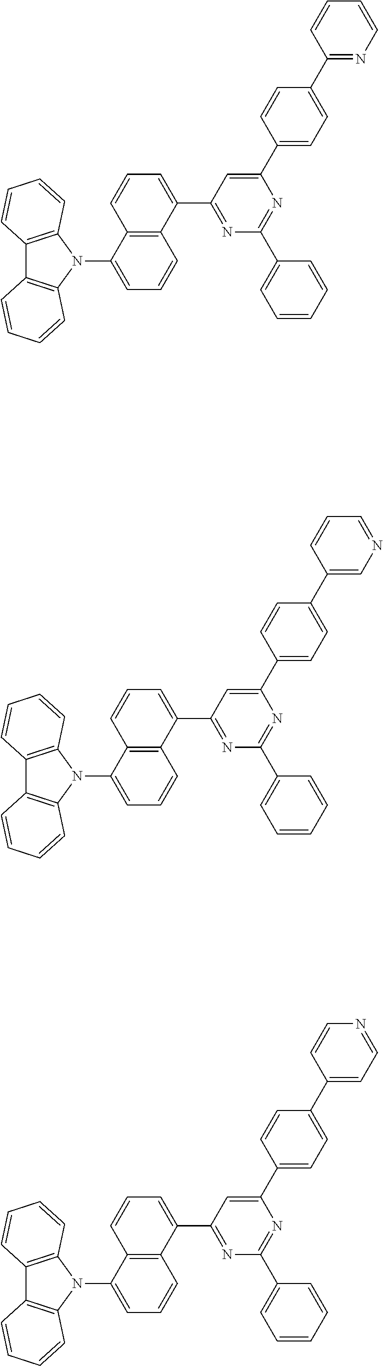 [SMILES: c1ccc(-c2nc(-c3ccc(-c4ccccn4)cc3)cc(-c3cccc4c(-n5c6ccccc6c6ccccc65)cccc34)n2)cc1 ; c1ccc(-c2nc(-c3ccc(-c4cccnc4)cc3)cc(-c3cccc4c(-n5c6ccccc6c6ccccc65)cccc34)n2)cc1 ; c1ccc(-c2nc(-c3ccc(-c4ccncc4)cc3)cc(-c3cccc4c(-n5c6ccccc6c6ccccc65)cccc34)n2)cc1]

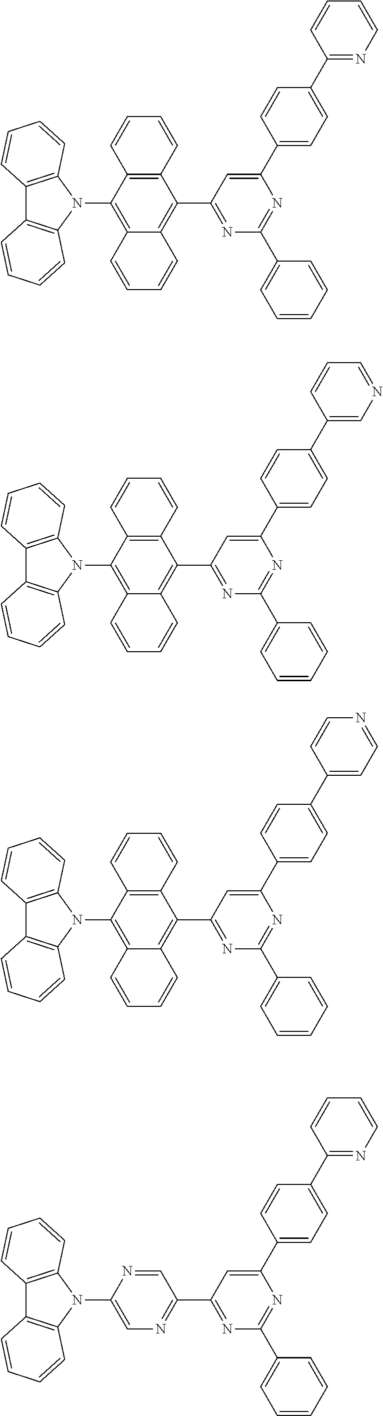

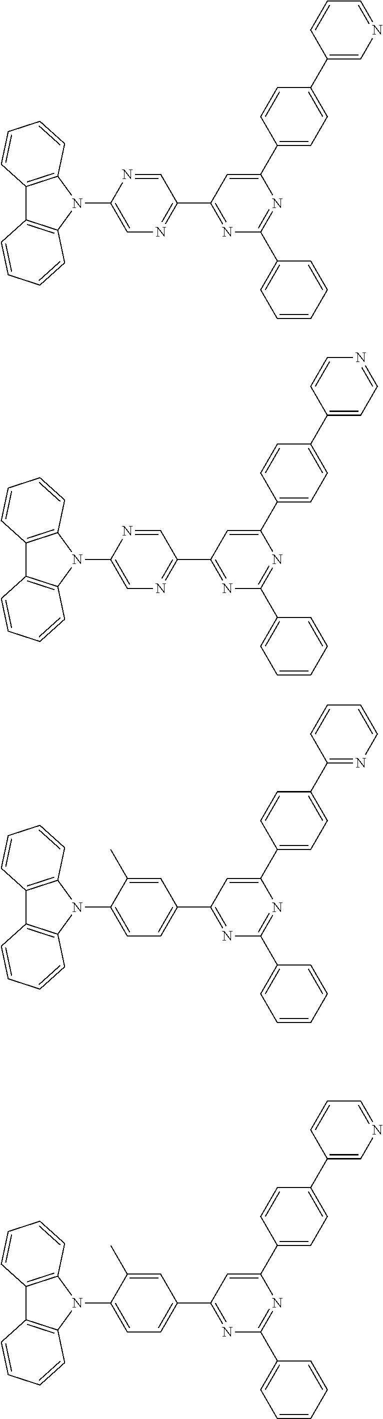

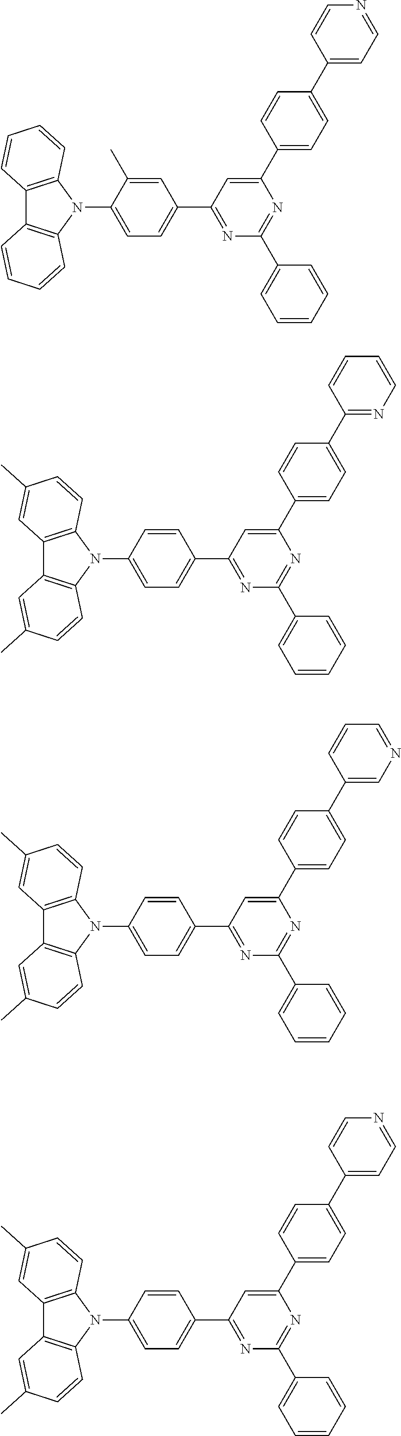 [SMILES: Cc1cc(-c2cc(-c3ccc(-c4ccncc4)cc3)nc(-c3ccccc3)n2)ccc1-n1c2ccccc2c2ccccc21 ; Cc1ccc2c(c1)c1cc(C)ccc1n2-c1ccc(-c2cc(-c3ccc(-c4ccccn4)cc3)nc(-c3ccccc3)n2)cc1 ; Cc1ccc2c(c1)c1cc(C)ccc1n2-c1ccc(-c2cc(-c3ccc(-c4cccnc4)cc3)nc(-c3ccccc3)n2)cc1 ; Cc1ccc2c(c1)c1cc(C)ccc1n2-c1ccc(-c2cc(-c3ccc(-c4ccncc4)cc3)nc(-c3ccccc3)n2)cc1]

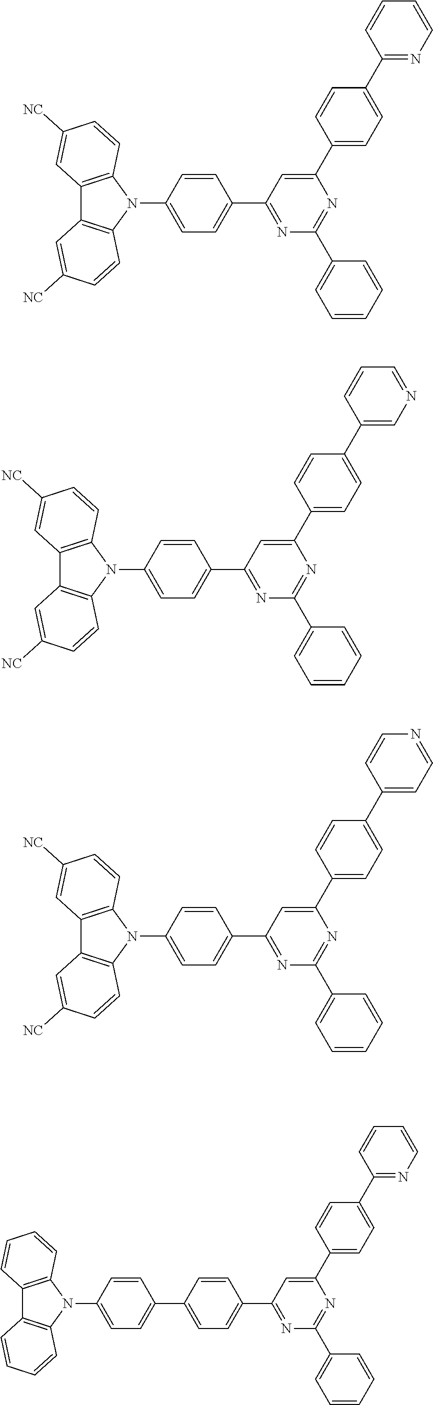 [SMILES: N#Cc1ccc2c(c1)c1cc(C#N)ccc1n2-c1ccc(-c2cc(-c3ccc(-c4ccccn4)cc3)nc(-c3ccccc3)n2)cc1 ; N#Cc1ccc2c(c1)c1cc(C#N)ccc1n2-c1ccc(-c2cc(-c3ccc(-c4cccnc4)cc3)nc(-c3ccccc3)n2)cc1 ; N#Cc1ccc2c(c1)c1cc(C#N)ccc1n2-c1ccc(-c2cc(-c3ccc(-c4ccncc4)cc3)nc(-c3ccccc3)n2)cc1 ; c1ccc(-c2nc(-c3ccc(-c4ccc(-n5c6ccccc6c6ccccc65)cc4)cc3)cc(-c3ccc(-c4ccccn4)cc3)n2)cc1]

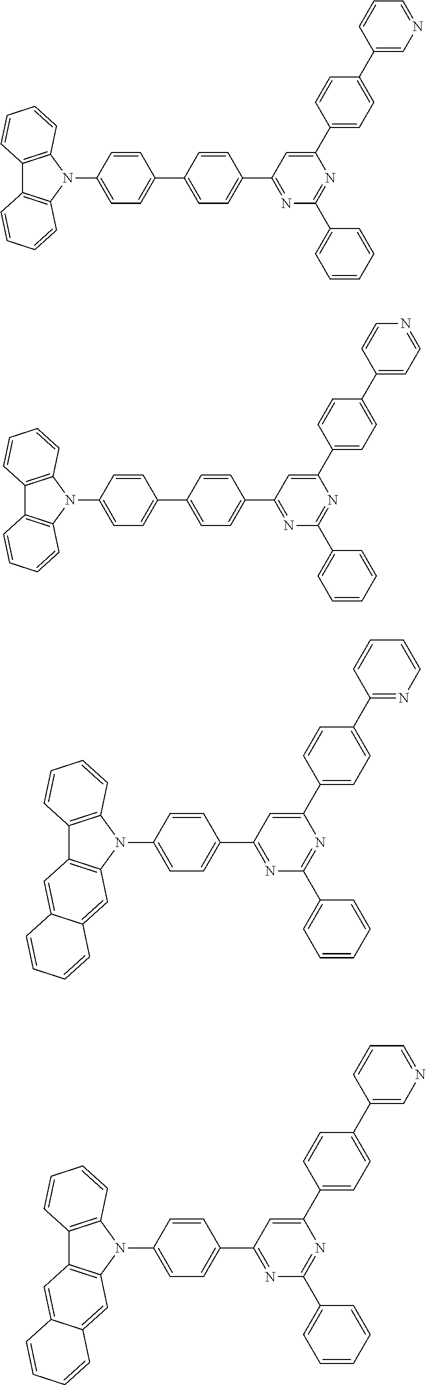

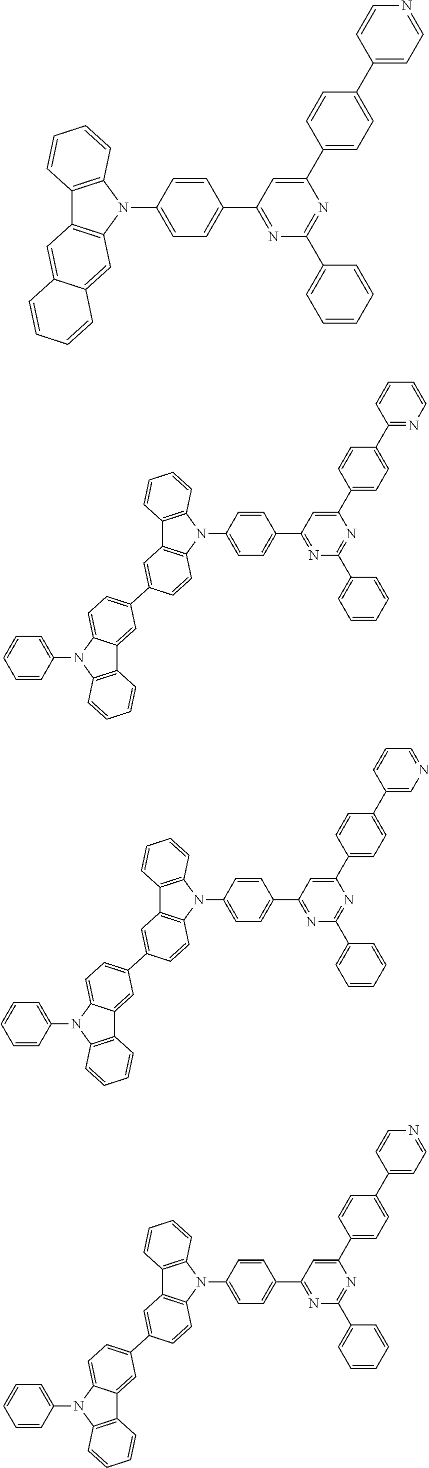 [SMILES: c1ccc(-c2nc(-c3ccc(-c4ccncc4)cc3)cc(-c3ccc(-n4c5ccccc5c5cc6ccccc6cc54)cc3)n2)cc1 ; c1ccc(-c2nc(-c3ccc(-c4ccccn4)cc3)cc(-c3ccc(-n4c5ccccc5c5cc(-c6ccc7c(c6)c6ccccc6n7-c6ccccc6)ccc54)cc3)n2)cc1 ; c1ccc(-c2nc(-c3ccc(-c4cccnc4)cc3)cc(-c3ccc(-n4c5ccccc5c5cc(-c6ccc7c(c6)c6ccccc6n7-c6ccccc6)ccc54)cc3)n2)cc1 ; c1ccc(-c2nc(-c3ccc(-c4ccncc4)cc3)cc(-c3ccc(-n4c5ccccc5c5cc(-c6ccc7c(c6)c6ccccc6n7-c6ccccc6)ccc54)cc3)n2)cc1]

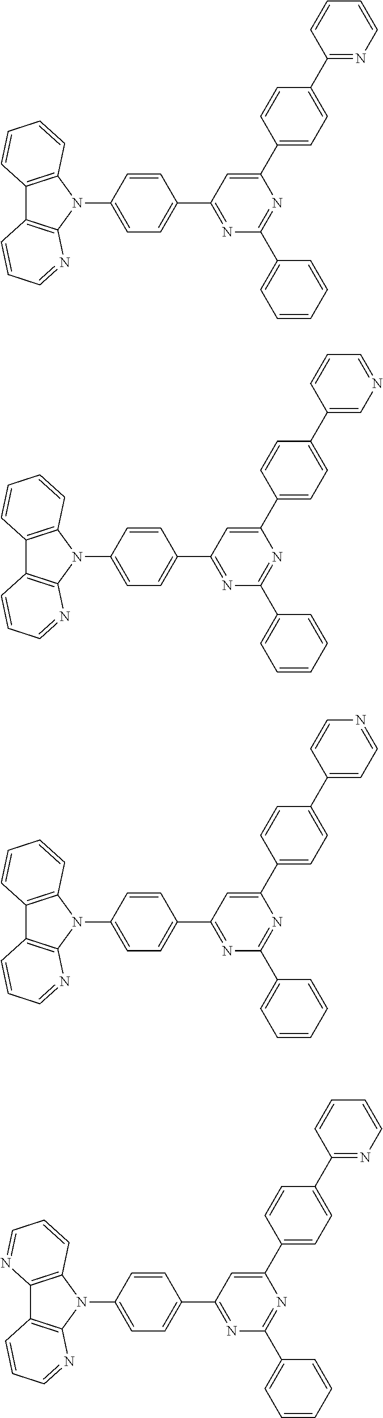

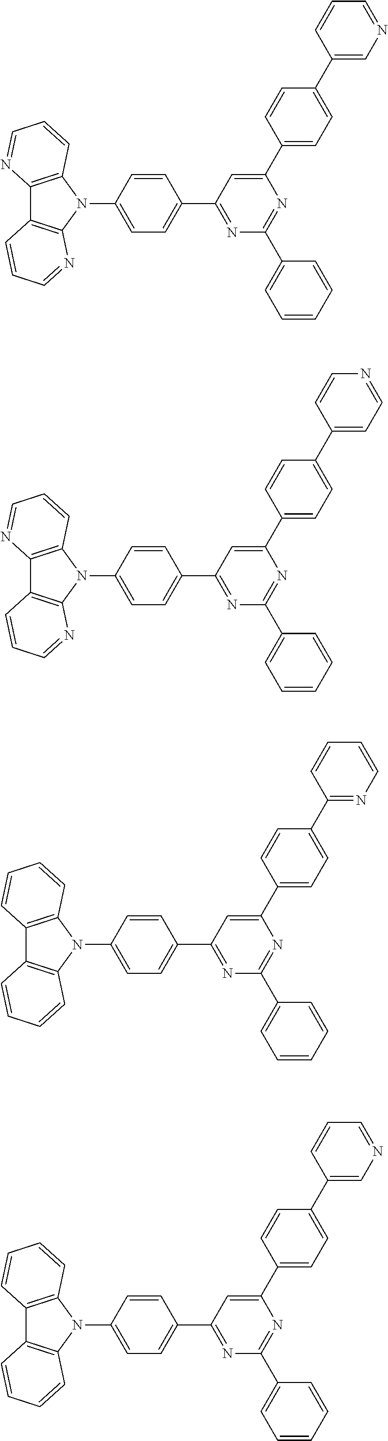 [SMILES: c1ccc(-c2nc(-c3ccc(-c4cccnc4)cc3)cc(-c3ccc(-n4c5cccnc5c5cccnc54)cc3)n2)cc1 ; c1ccc(-c2nc(-c3ccc(-c4ccncc4)cc3)cc(-c3ccc(-n4c5cccnc5c5cccnc54)cc3)n2)cc1 ; c1ccc(-c2nc(-c3ccc(-c4ccccn4)cc3)cc(-c3ccc(-n4c5ccccc5c5ccccc54)cc3)n2)cc1 ; c1ccc(-c2nc(-c3ccc(-c4cccnc4)cc3)cc(-c3ccc(-n4c5ccccc5c5ccccc54)cc3)n2)cc1]

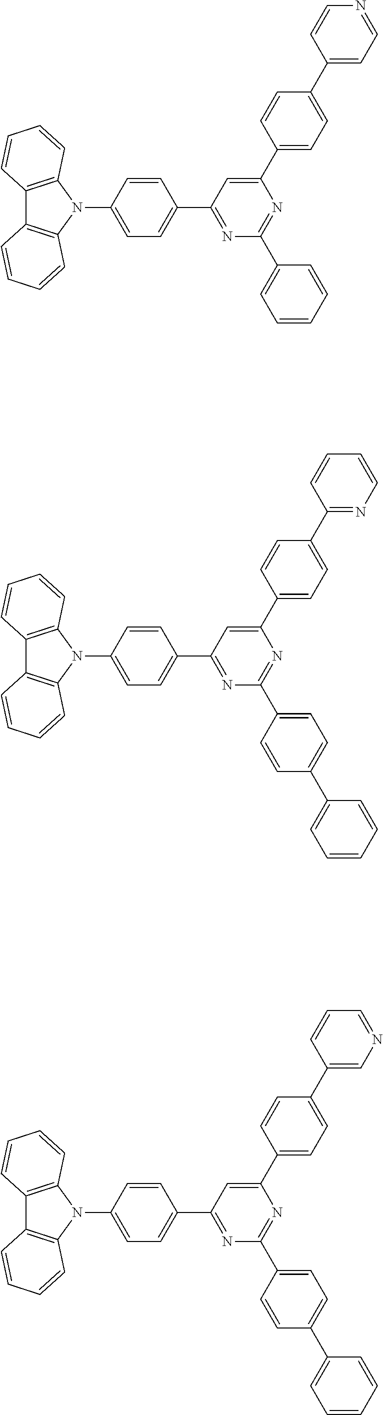 [SMILES: c1ccc(-c2nc(-c3ccc(-c4ccncc4)cc3)cc(-c3ccc(-n4c5ccccc5c5ccccc54)cc3)n2)cc1 ; c1ccc(-c2ccc(-c3nc(-c4ccc(-c5ccccn5)cc4)cc(-c4ccc(-n5c6ccccc6c6ccccc65)cc4)n3)cc2)cc1 ; c1ccc(-c2ccc(-c3nc(-c4ccc(-c5cccnc5)cc4)cc(-c4ccc(-n5c6ccccc6c6ccccc65)cc4)n3)cc2)cc1]

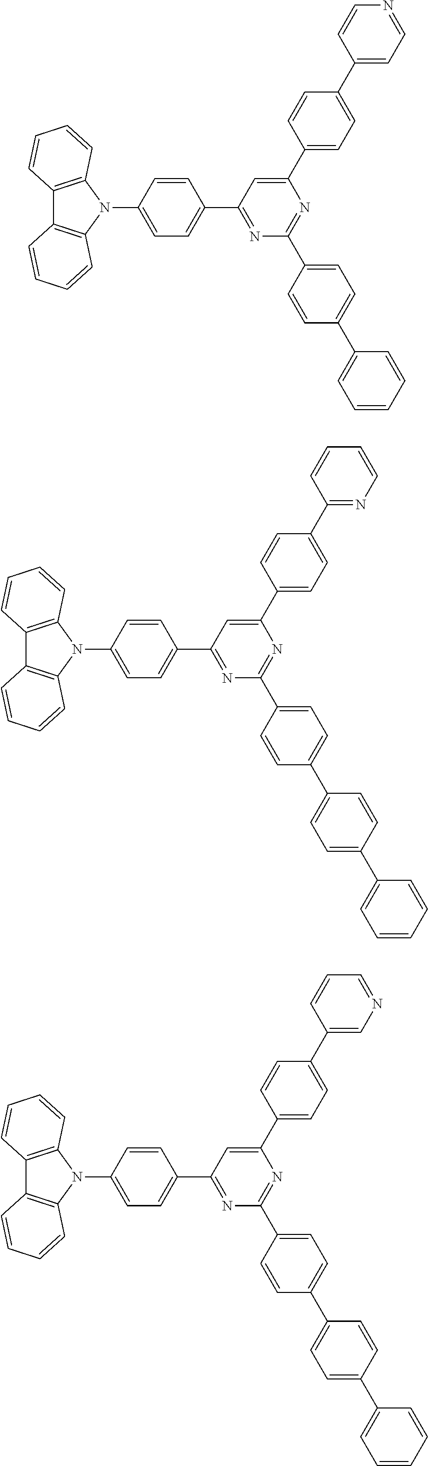

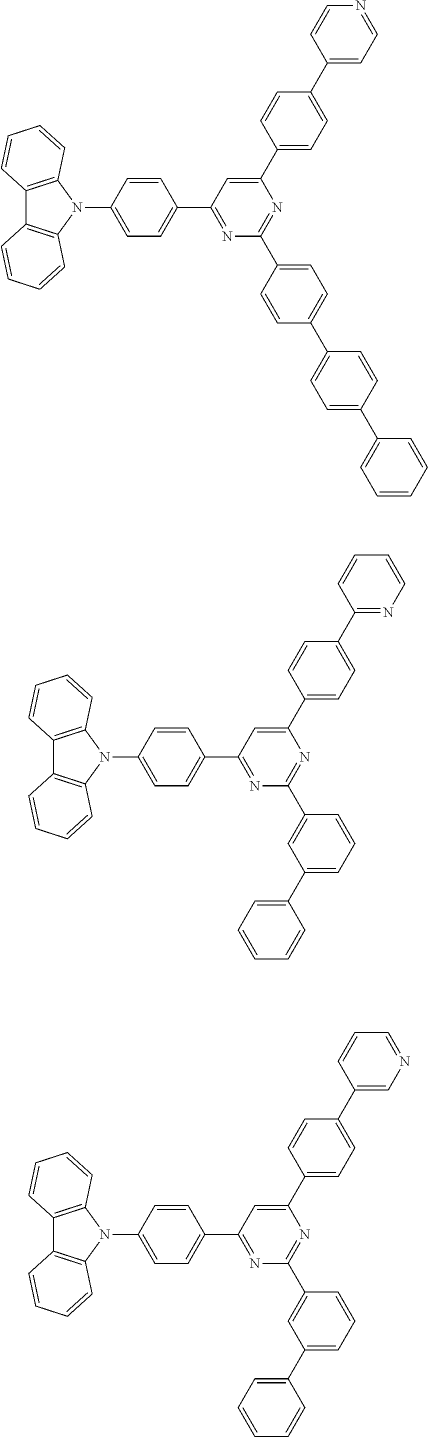 [SMILES: c1ccc(-c2ccc(-c3ccc(-c4nc(-c5ccc(-c6ccncc6)cc5)cc(-c5ccc(-n6c7ccccc7c7ccccc76)cc5)n4)cc3)cc2)cc1 ; c1ccc(-c2cccc(-c3nc(-c4ccc(-c5ccccn5)cc4)cc(-c4ccc(-n5c6ccccc6c6ccccc65)cc4)n3)c2)cc1 ; c1ccc(-c2cccc(-c3nc(-c4ccc(-c5cccnc5)cc4)cc(-c4ccc(-n5c6ccccc6c6ccccc65)cc4)n3)c2)cc1]

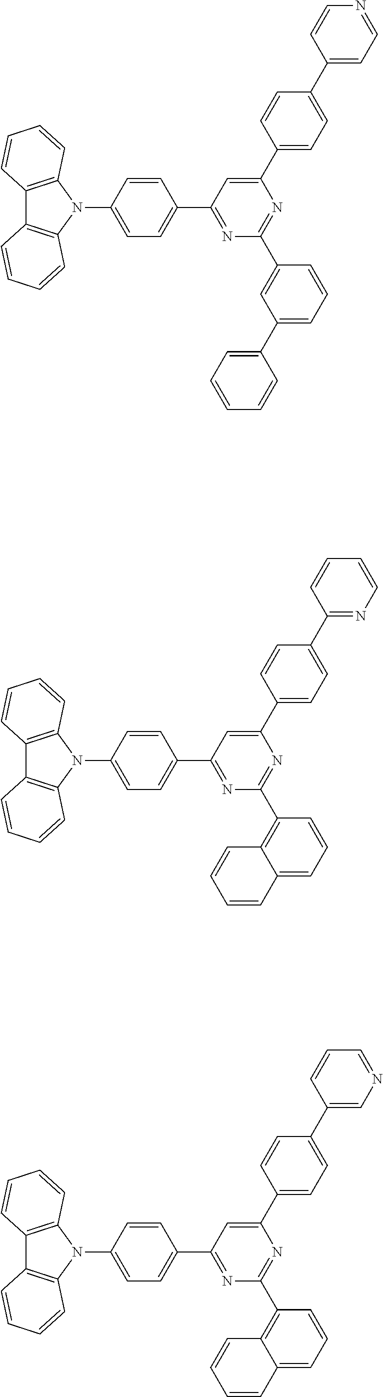 [SMILES: c1ccc(-c2cccc(-c3nc(-c4ccc(-c5ccncc5)cc4)cc(-c4ccc(-n5c6ccccc6c6ccccc65)cc4)n3)c2)cc1 ; c1ccc(-c2ccc(-c3cc(-c4ccc(-n5c6ccccc6c6ccccc65)cc4)nc(-c4cccc5ccccc45)n3)cc2)nc1 ; c1cncc(-c2ccc(-c3cc(-c4ccc(-n5c6ccccc6c6ccccc65)cc4)nc(-c4cccc5ccccc45)n3)cc2)c1]

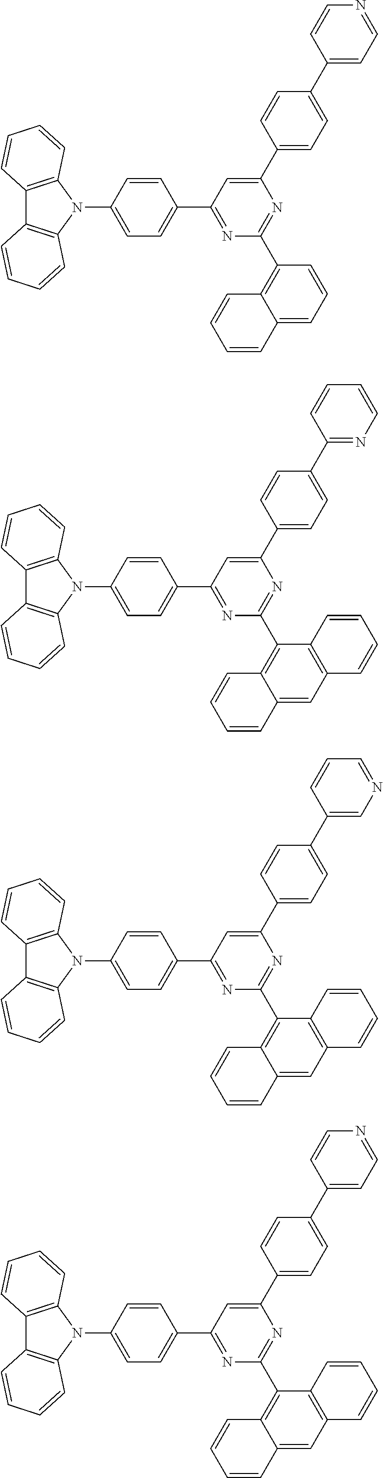 [SMILES: c1ccc2c(-c3nc(-c4ccc(-c5ccncc5)cc4)cc(-c4ccc(-n5c6ccccc6c6ccccc65)cc4)n3)cccc2c1 ; c1ccc(-c2ccc(-c3cc(-c4ccc(-n5c6ccccc6c6ccccc65)cc4)nc(-c4c5ccccc5cc5ccccc45)n3)cc2)nc1 ; c1cncc(-c2ccc(-c3cc(-c4ccc(-n5c6ccccc6c6ccccc65)cc4)nc(-c4c5ccccc5cc5ccccc45)n3)cc2)c1 ; c1ccc2c(-c3nc(-c4ccc(-c5ccncc5)cc4)cc(-c4ccc(-n5c6ccccc6c6ccccc65)cc4)n3)c3ccccc3cc2c1]

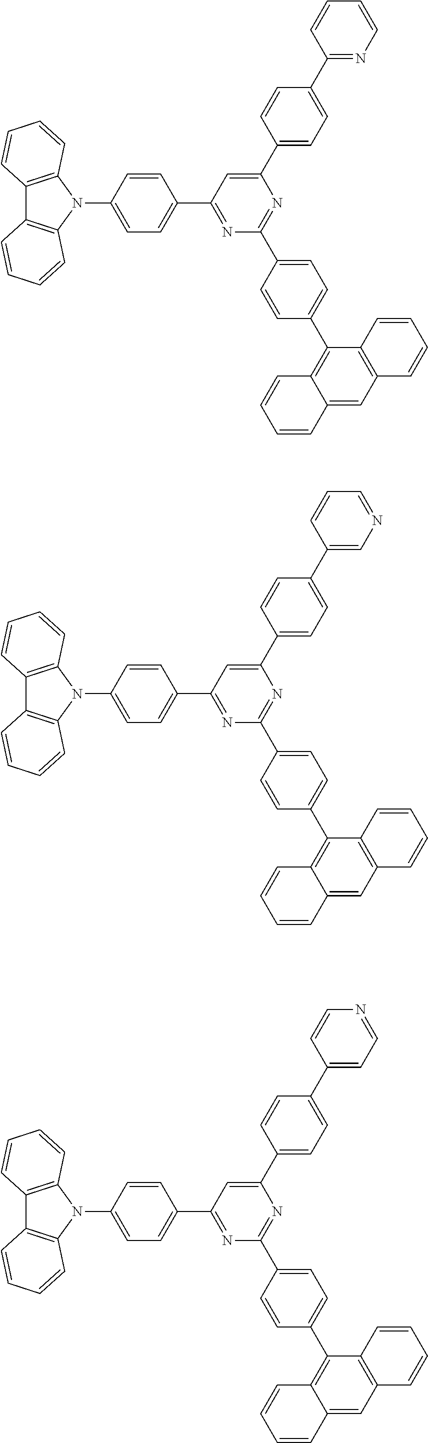 [SMILES: c1ccc(-c2ccc(-c3cc(-c4ccc(-n5c6ccccc6c6ccccc65)cc4)nc(-c4ccc(-c5c6ccccc6cc6ccccc56)cc4)n3)cc2)nc1 ; c1cncc(-c2ccc(-c3cc(-c4ccc(-n5c6ccccc6c6ccccc65)cc4)nc(-c4ccc(-c5c6ccccc6cc6ccccc56)cc4)n3)cc2)c1 ; c1ccc2c(-c3ccc(-c4nc(-c5ccc(-c6ccncc6)cc5)cc(-c5ccc(-n6c7ccccc7c7ccccc76)cc5)n4)cc3)c3ccccc3cc2c1]

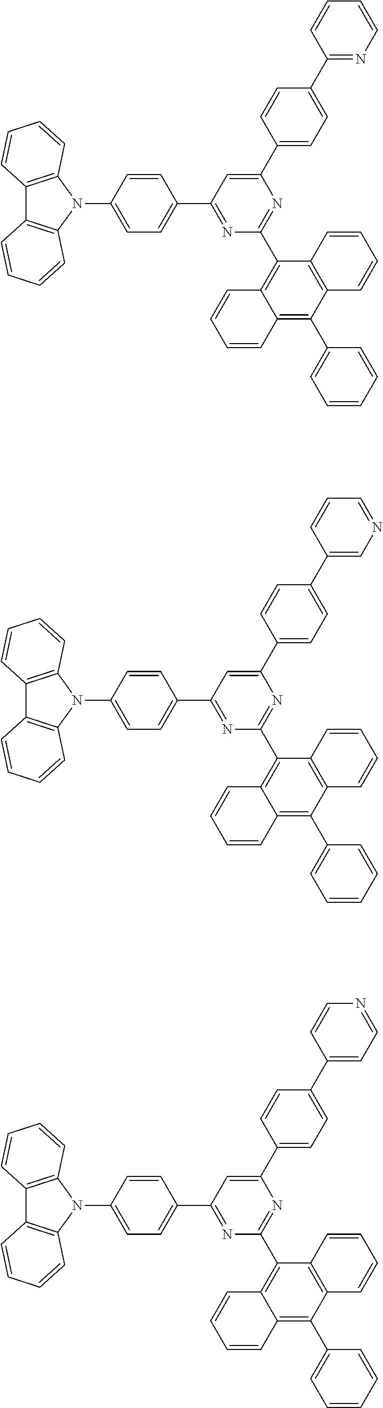 [SMILES: c1ccc(-c2c3ccccc3c(-c3nc(-c4ccc(-c5ccccn5)cc4)cc(-c4ccc(-n5c6ccccc6c6ccccc65)cc4)n3)c3ccccc23)cc1 ; c1ccc(-c2c3ccccc3c(-c3nc(-c4ccc(-c5cccnc5)cc4)cc(-c4ccc(-n5c6ccccc6c6ccccc65)cc4)n3)c3ccccc23)cc1 ; c1ccc(-c2c3ccccc3c(-c3nc(-c4ccc(-c5ccncc5)cc4)cc(-c4ccc(-n5c6ccccc6c6ccccc65)cc4)n3)c3ccccc23)cc1]

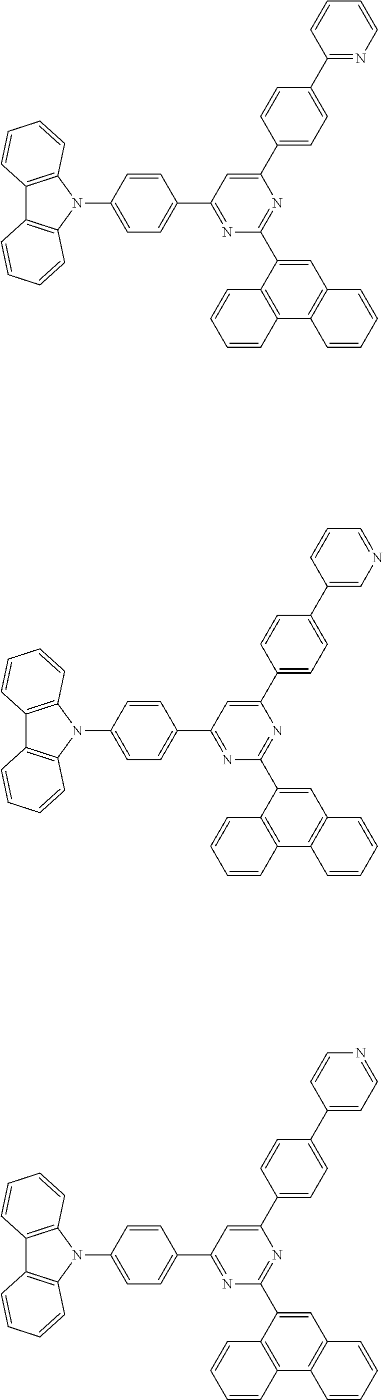 [SMILES: c1ccc(-c2ccc(-c3cc(-c4ccc(-n5c6ccccc6c6ccccc65)cc4)nc(-c4cc5ccccc5c5ccccc45)n3)cc2)nc1 ; c1cncc(-c2ccc(-c3cc(-c4ccc(-n5c6ccccc6c6ccccc65)cc4)nc(-c4cc5ccccc5c5ccccc45)n3)cc2)c1 ; c1ccc2c(c1)cc(-c1nc(-c3ccc(-c4ccncc4)cc3)cc(-c3ccc(-n4c5ccccc5c5ccccc54)cc3)n1)c1ccccc12]

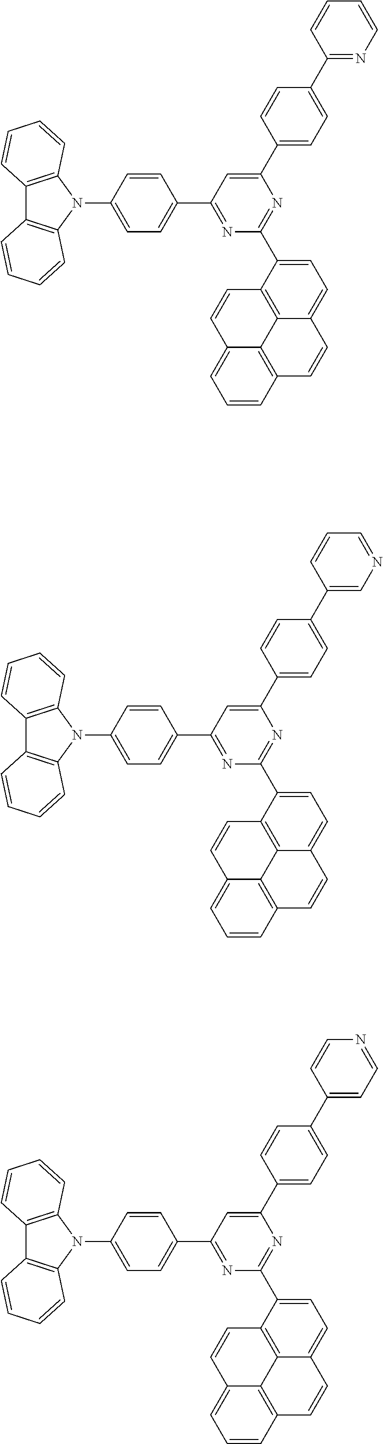 [SMILES: c1ccc(-c2ccc(-c3cc(-c4ccc(-n5c6ccccc6c6ccccc65)cc4)nc(-c4ccc5ccc6cccc7ccc4c5c67)n3)cc2)nc1 ; c1cncc(-c2ccc(-c3cc(-c4ccc(-n5c6ccccc6c6ccccc65)cc4)nc(-c4ccc5ccc6cccc7ccc4c5c67)n3)cc2)c1 ; c1cc2ccc3ccc(-c4nc(-c5ccc(-c6ccncc6)cc5)cc(-c5ccc(-n6c7ccccc7c7ccccc76)cc5)n4)c4ccc(c1)c2c34]

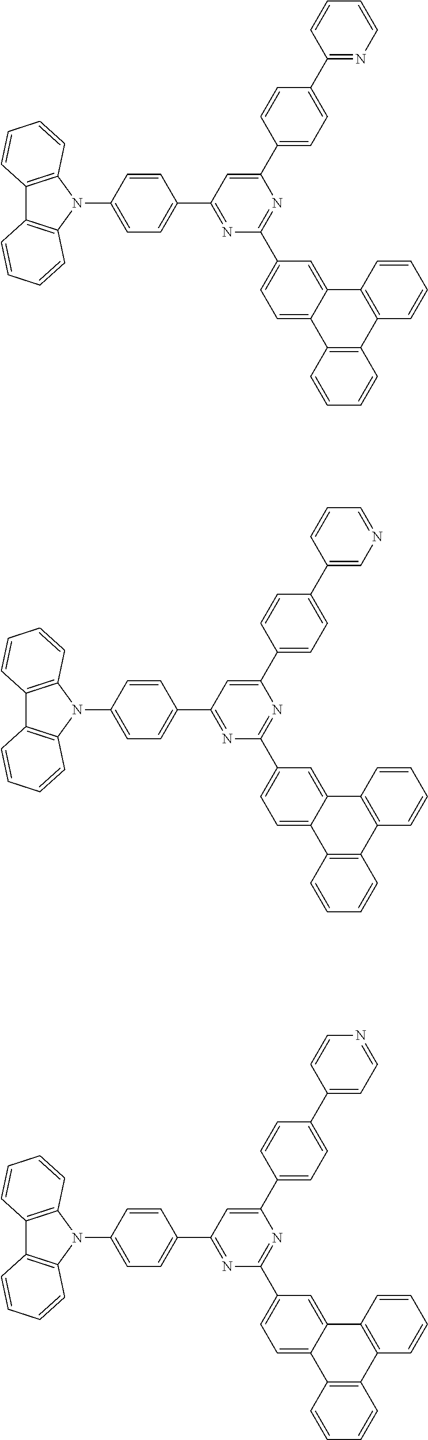 [SMILES: c1ccc(-c2ccc(-c3cc(-c4ccc(-n5c6ccccc6c6ccccc65)cc4)nc(-c4ccc5c6ccccc6c6ccccc6c5c4)n3)cc2)nc1 ; c1cncc(-c2ccc(-c3cc(-c4ccc(-n5c6ccccc6c6ccccc65)cc4)nc(-c4ccc5c6ccccc6c6ccccc6c5c4)n3)cc2)c1 ; c1ccc2c(c1)c1ccccc1c1cc(-c3nc(-c4ccc(-c5ccncc5)cc4)cc(-c4ccc(-n5c6ccccc6c6ccccc65)cc4)n3)ccc21]

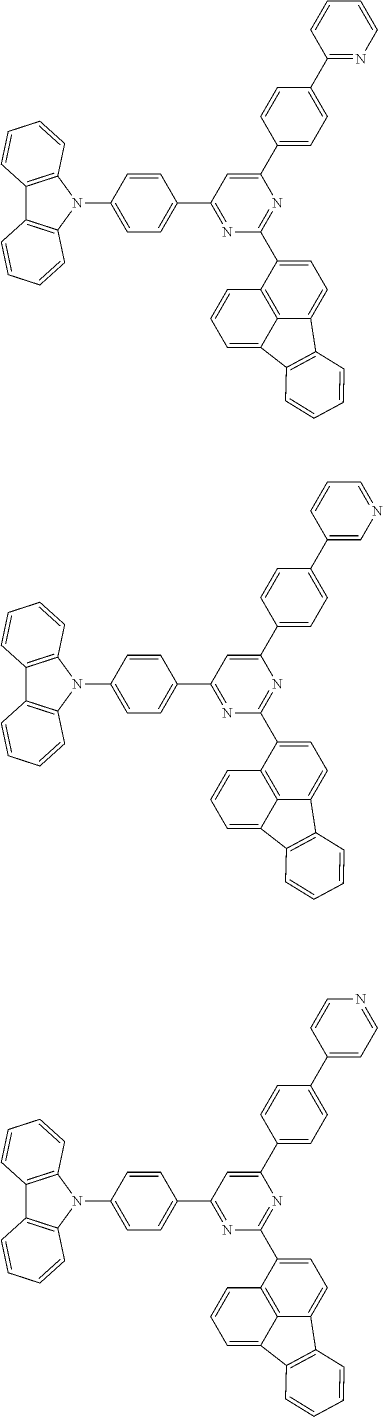 [SMILES: c1ccc(-c2ccc(-c3cc(-c4ccc(-n5c6ccccc6c6ccccc65)cc4)nc(-c4ccc5c6c(cccc46)-c4ccccc4-5)n3)cc2)nc1 ; c1cncc(-c2ccc(-c3cc(-c4ccc(-n5c6ccccc6c6ccccc65)cc4)nc(-c4ccc5c6c(cccc46)-c4ccccc4-5)n3)cc2)c1 ; c1ccc2c(c1)-c1cccc3c(-c4nc(-c5ccc(-c6ccncc6)cc5)cc(-c5ccc(-n6c7ccccc7c7ccccc76)cc5)n4)ccc-2c13]

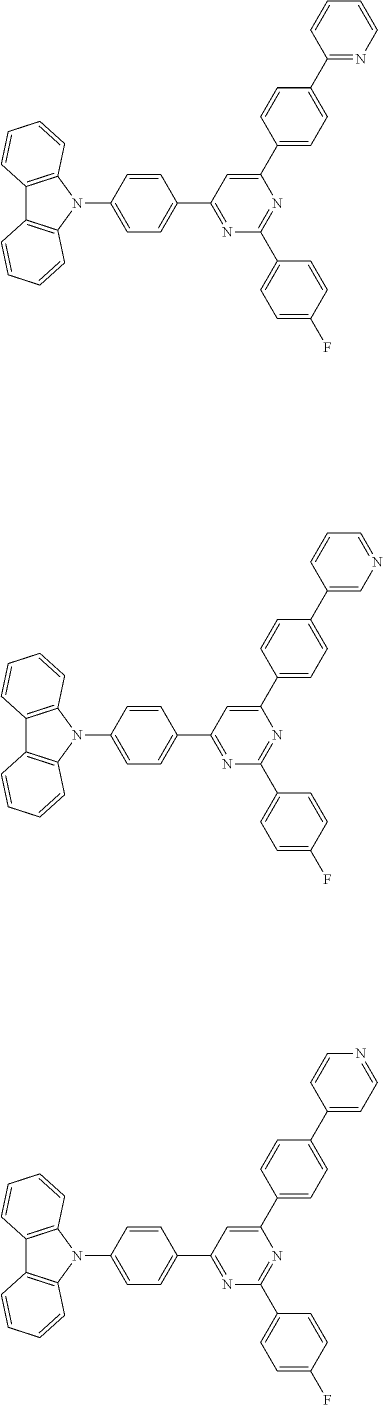 [SMILES: Fc1ccc(-c2nc(-c3ccc(-c4ccccn4)cc3)cc(-c3ccc(-n4c5ccccc5c5ccccc54)cc3)n2)cc1 ; Fc1ccc(-c2nc(-c3ccc(-c4cccnc4)cc3)cc(-c3ccc(-n4c5ccccc5c5ccccc54)cc3)n2)cc1 ; Fc1ccc(-c2nc(-c3ccc(-c4ccncc4)cc3)cc(-c3ccc(-n4c5ccccc5c5ccccc54)cc3)n2)cc1]

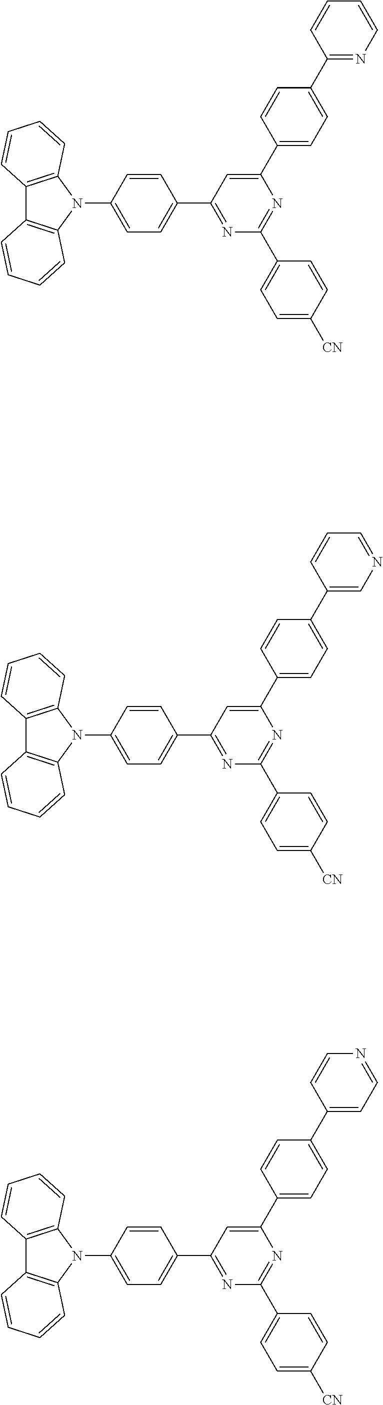 [SMILES: N#Cc1ccc(-c2nc(-c3ccc(-c4ccccn4)cc3)cc(-c3ccc(-n4c5ccccc5c5ccccc54)cc3)n2)cc1 ; N#Cc1ccc(-c2nc(-c3ccc(-c4cccnc4)cc3)cc(-c3ccc(-n4c5ccccc5c5ccccc54)cc3)n2)cc1 ; N#Cc1ccc(-c2nc(-c3ccc(-c4ccncc4)cc3)cc(-c3ccc(-n4c5ccccc5c5ccccc54)cc3)n2)cc1]

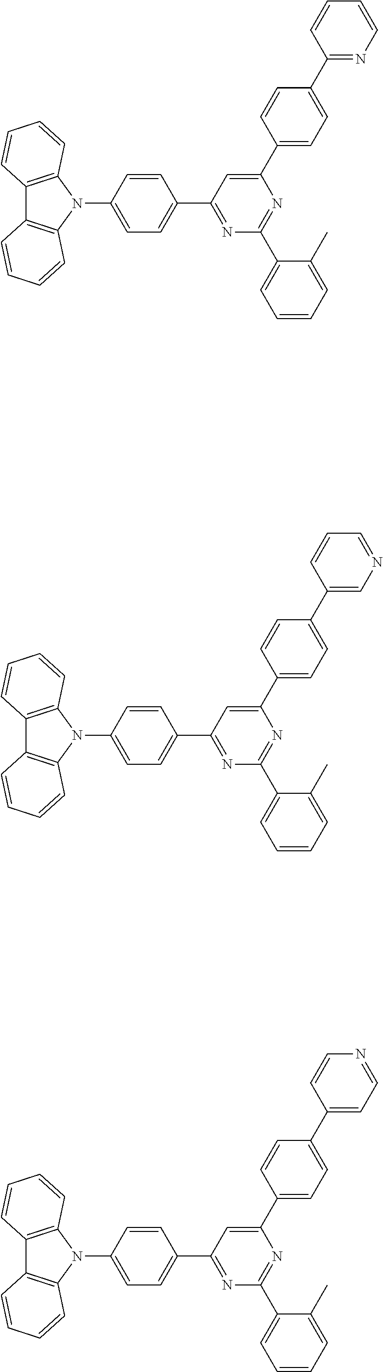 [SMILES: Cc1ccccc1-c1nc(-c2ccc(-c3ccccn3)cc2)cc(-c2ccc(-n3c4ccccc4c4ccccc43)cc2)n1 ; Cc1ccccc1-c1nc(-c2ccc(-c3cccnc3)cc2)cc(-c2ccc(-n3c4ccccc4c4ccccc43)cc2)n1 ; Cc1ccccc1-c1nc(-c2ccc(-c3ccncc3)cc2)cc(-c2ccc(-n3c4ccccc4c4ccccc43)cc2)n1]

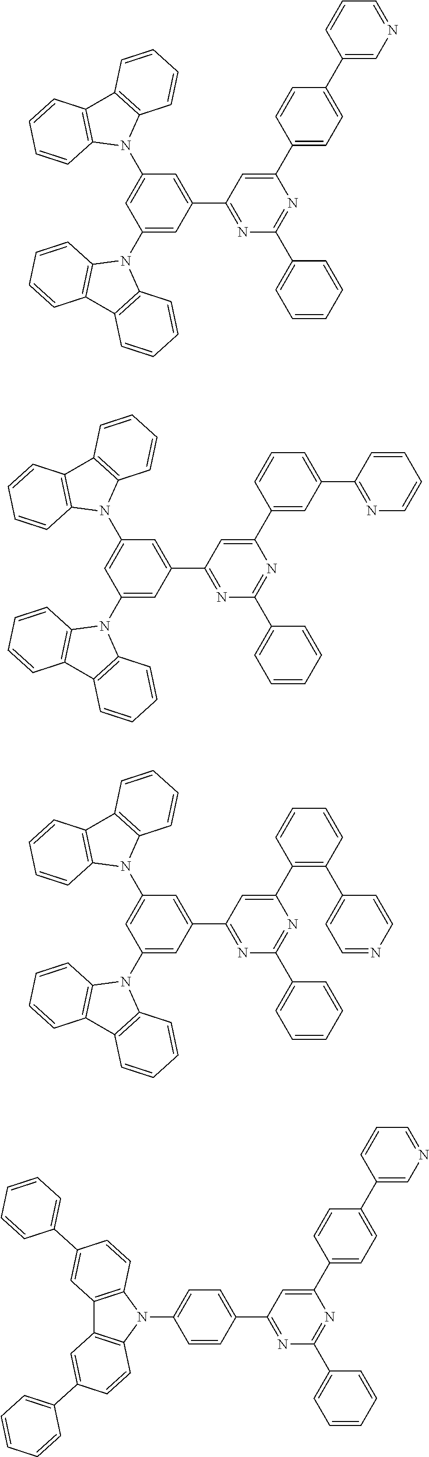 [SMILES: c1ccc(-c2nc(-c3ccc(-c4cccnc4)cc3)cc(-c3cc(-n4c5ccccc5c5ccccc54)cc(-n4c5ccccc5c5ccccc54)c3)n2)cc1 ; c1ccc(-c2nc(-c3cccc(-c4ccccn4)c3)cc(-c3cc(-n4c5ccccc5c5ccccc54)cc(-n4c5ccccc5c5ccccc54)c3)n2)cc1 ; c1ccc(-c2nc(-c3cc(-n4c5ccccc5c5ccccc54)cc(-n4c5ccccc5c5ccccc54)c3)cc(-c3ccccc3-c3ccncc3)n2)cc1 ; c1ccc(-c2ccc3c(c2)c2cc(-c4ccccc4)ccc2n3-c2ccc(-c3cc(-c4ccc(-c5cccnc5)cc4)nc(-c4ccccc4)n3)cc2)cc1]

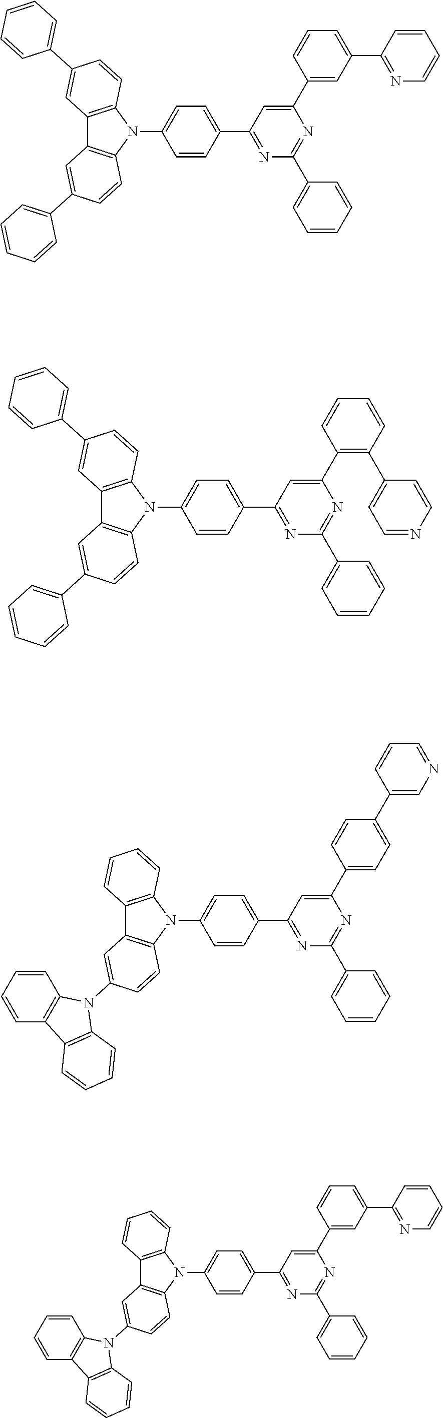 [SMILES: c1ccc(-c2ccc3c(c2)c2cc(-c4ccccc4)ccc2n3-c2ccc(-c3cc(-c4cccc(-c5ccccn5)c4)nc(-c4ccccc4)n3)cc2)cc1 ; c1ccc(-c2ccc3c(c2)c2cc(-c4ccccc4)ccc2n3-c2ccc(-c3cc(-c4ccccc4-c4ccncc4)nc(-c4ccccc4)n3)cc2)cc1 ; c1ccc(-c2nc(-c3ccc(-c4cccnc4)cc3)cc(-c3ccc(-n4c5ccccc5c5cc(-n6c7ccccc7c7ccccc76)ccc54)cc3)n2)cc1 ; c1ccc(-c2nc(-c3ccc(-n4c5ccccc5c5cc(-n6c7ccccc7c7ccccc76)ccc54)cc3)cc(-c3cccc(-c4ccccn4)c3)n2)cc1]

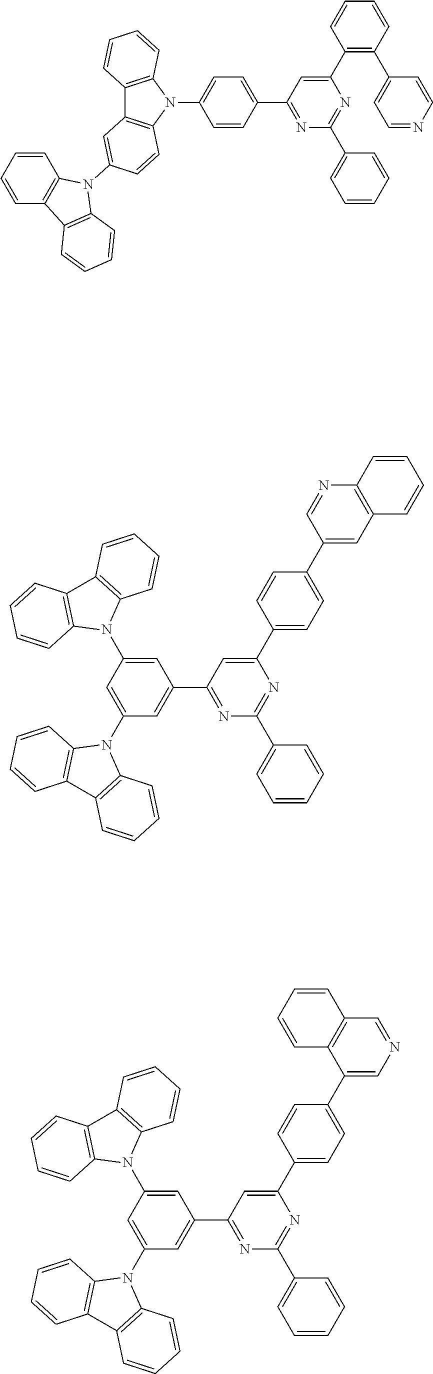 [SMILES: c1ccc(-c2nc(-c3ccc(-n4c5ccccc5c5cc(-n6c7ccccc7c7ccccc76)ccc54)cc3)cc(-c3ccccc3-c3ccncc3)n2)cc1 ; c1ccc(-c2nc(-c3ccc(-c4cnc5ccccc5c4)cc3)cc(-c3cc(-n4c5ccccc5c5ccccc54)cc(-n4c5ccccc5c5ccccc54)c3)n2)cc1 ; c1ccc(-c2nc(-c3ccc(-c4cncc5ccccc45)cc3)cc(-c3cc(-n4c5ccccc5c5ccccc54)cc(-n4c5ccccc5c5ccccc54)c3)n2)cc1]

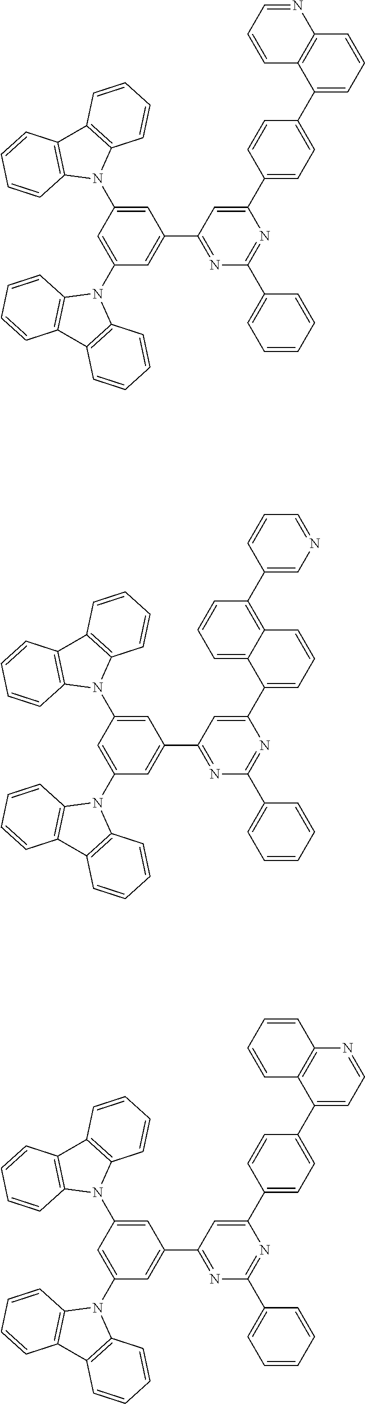 [SMILES: c1ccc(-c2nc(-c3ccc(-c4cccc5ncccc45)cc3)cc(-c3cc(-n4c5ccccc5c5ccccc54)cc(-n4c5ccccc5c5ccccc54)c3)n2)cc1 ; c1ccc(-c2nc(-c3cc(-n4c5ccccc5c5ccccc54)cc(-n4c5ccccc5c5ccccc54)c3)cc(-c3cccc4c(-c5cccnc5)cccc34)n2)cc1 ; c1ccc(-c2nc(-c3ccc(-c4ccnc5ccccc45)cc3)cc(-c3cc(-n4c5ccccc5c5ccccc54)cc(-n4c5ccccc5c5ccccc54)c3)n2)cc1]

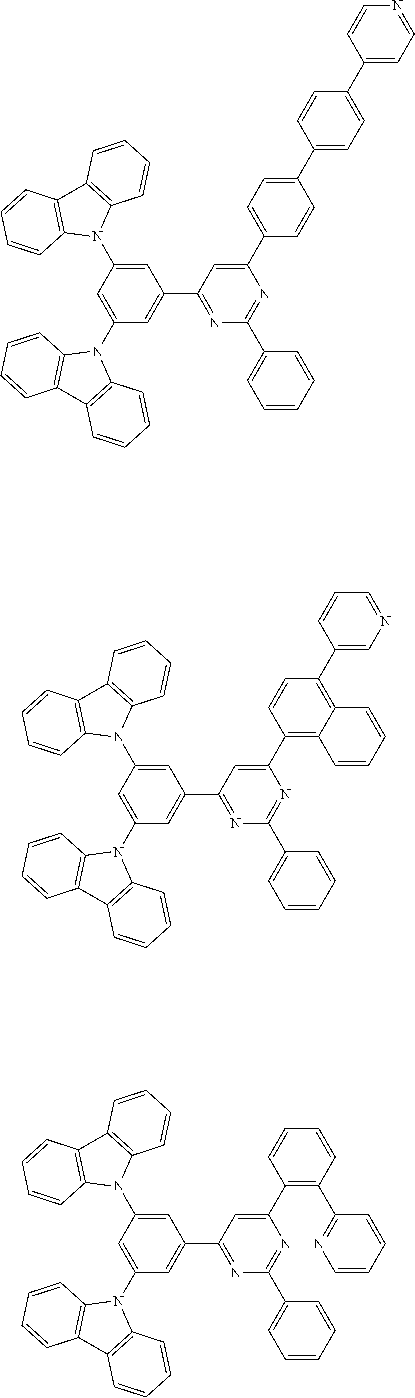 [SMILES: c1ccc(-c2nc(-c3ccc(-c4ccc(-c5ccncc5)cc4)cc3)cc(-c3cc(-n4c5ccccc5c5ccccc54)cc(-n4c5ccccc5c5ccccc54)c3)n2)cc1 ; c1ccc(-c2nc(-c3cc(-n4c5ccccc5c5ccccc54)cc(-n4c5ccccc5c5ccccc54)c3)cc(-c3ccc(-c4cccnc4)c4ccccc34)n2)cc1 ; c1ccc(-c2nc(-c3cc(-n4c5ccccc5c5ccccc54)cc(-n4c5ccccc5c5ccccc54)c3)cc(-c3ccccc3-c3ccccn3)n2)cc1]

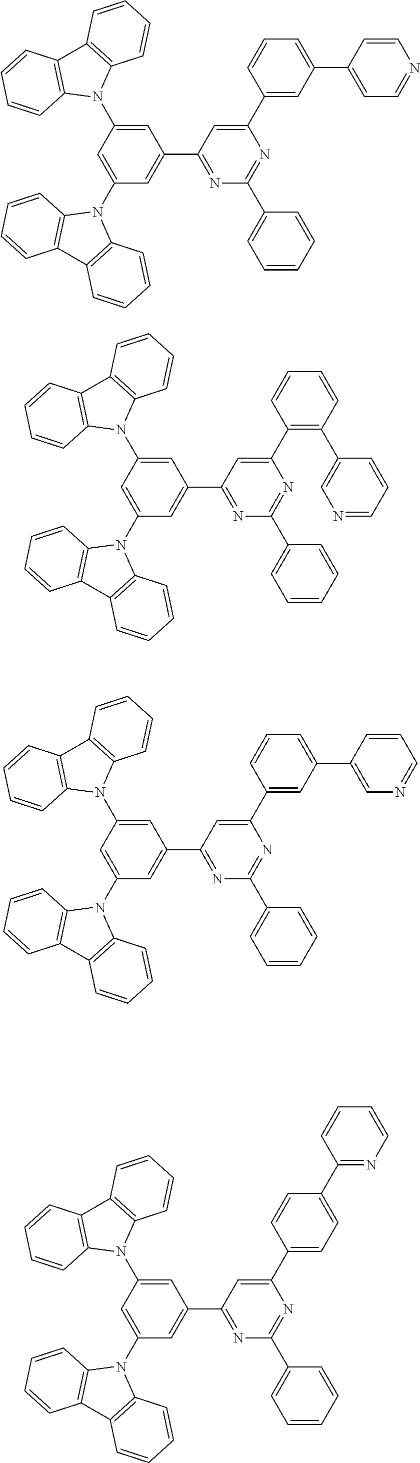

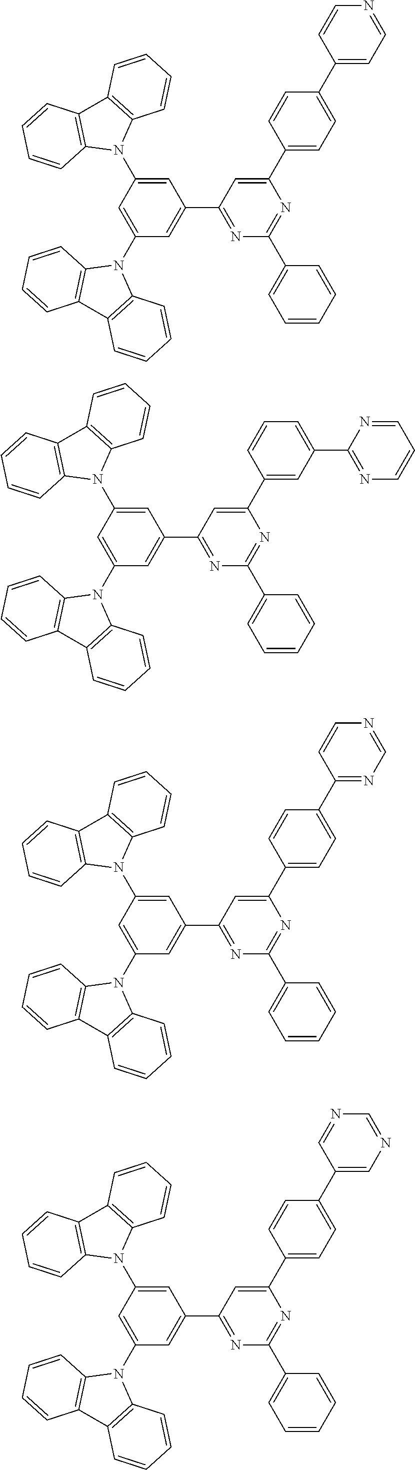

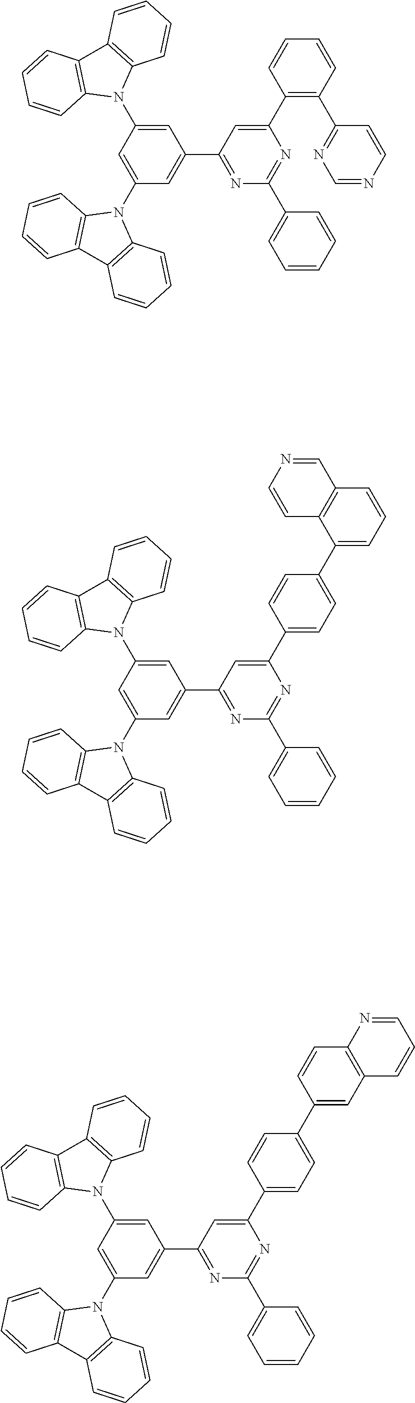 [SMILES: c1ccc(-c2nc(-c3cc(-n4c5ccccc5c5ccccc54)cc(-n4c5ccccc5c5ccccc54)c3)cc(-c3ccccc3-c3ccncn3)n2)cc1 ; c1ccc(-c2nc(-c3ccc(-c4cccc5cnccc45)cc3)cc(-c3cc(-n4c5ccccc5c5ccccc54)cc(-n4c5ccccc5c5ccccc54)c3)n2)cc1 ; c1ccc(-c2nc(-c3ccc(-c4ccc5ncccc5c4)cc3)cc(-c3cc(-n4c5ccccc5c5ccccc54)cc(-n4c5ccccc5c5ccccc54)c3)n2)cc1]

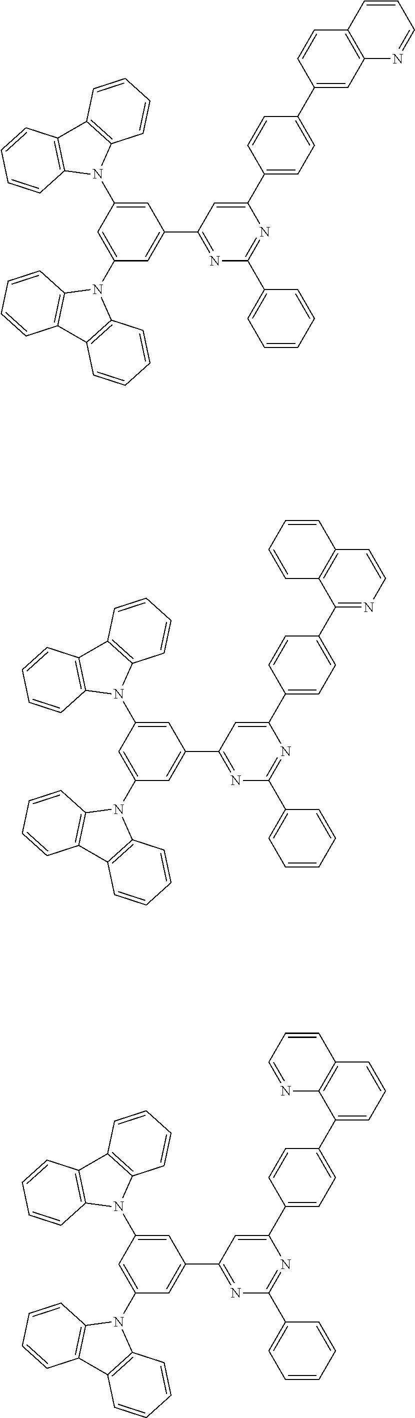 [SMILES: c1ccc(-c2nc(-c3ccc(-c4ccc5cccnc5c4)cc3)cc(-c3cc(-n4c5ccccc5c5ccccc54)cc(-n4c5ccccc5c5ccccc54)c3)n2)cc1 ; c1ccc(-c2nc(-c3ccc(-c4nccc5ccccc45)cc3)cc(-c3cc(-n4c5ccccc5c5ccccc54)cc(-n4c5ccccc5c5ccccc54)c3)n2)cc1 ; c1ccc(-c2nc(-c3ccc(-c4cccc5cccnc45)cc3)cc(-c3cc(-n4c5ccccc5c5ccccc54)cc(-n4c5ccccc5c5ccccc54)c3)n2)cc1]

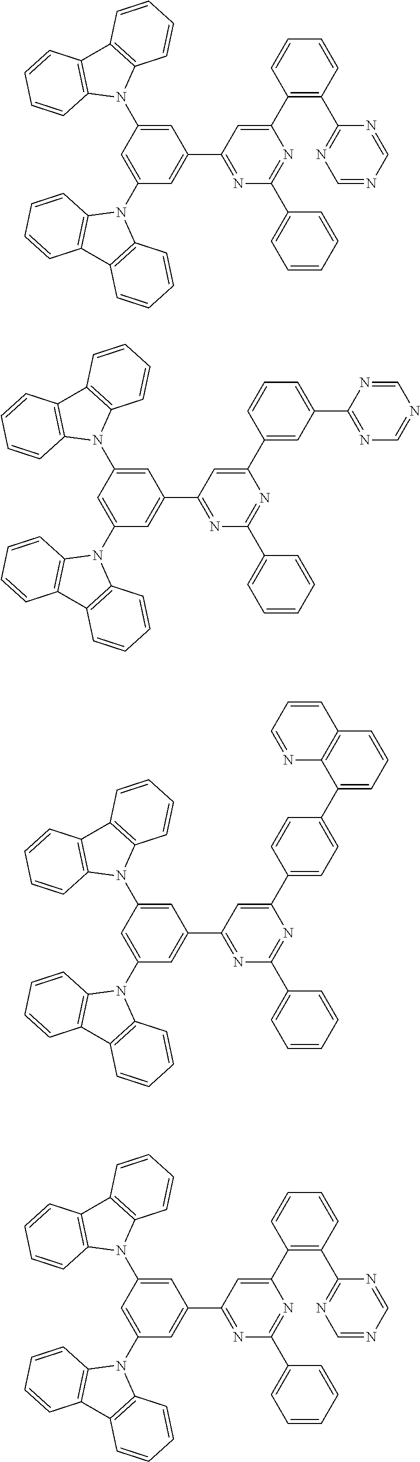

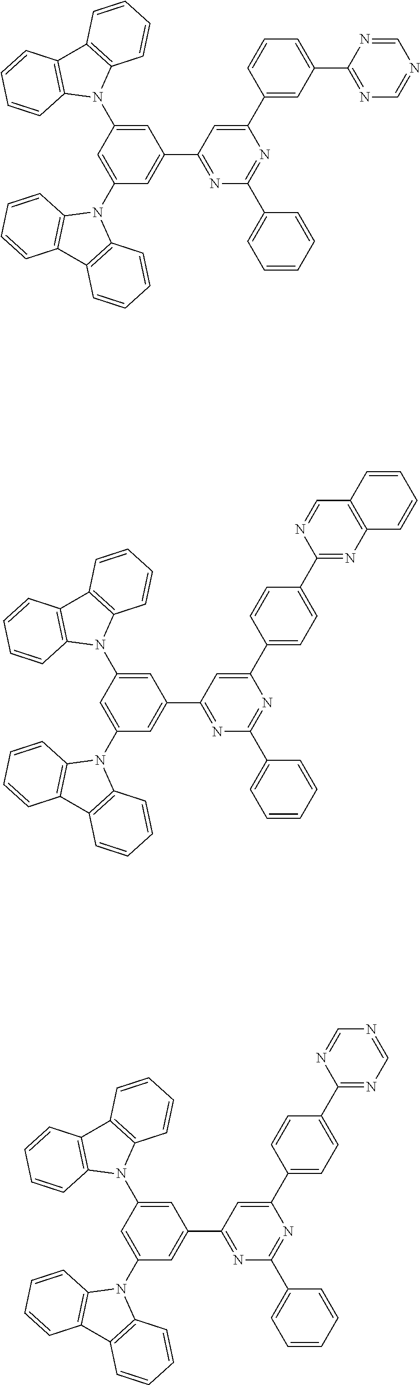 [SMILES: c1ccc(-c2nc(-c3cccc(-c4ncncn4)c3)cc(-c3cc(-n4c5ccccc5c5ccccc54)cc(-n4c5ccccc5c5ccccc54)c3)n2)cc1 ; c1ccc(-c2nc(-c3ccc(-c4ncc5ccccc5n4)cc3)cc(-c3cc(-n4c5ccccc5c5ccccc54)cc(-n4c5ccccc5c5ccccc54)c3)n2)cc1 ; c1ccc(-c2nc(-c3ccc(-c4ncncn4)cc3)cc(-c3cc(-n4c5ccccc5c5ccccc54)cc(-n4c5ccccc5c5ccccc54)c3)n2)cc1]

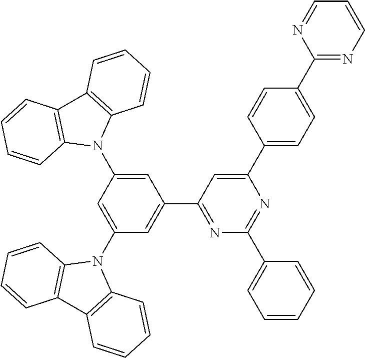 [SMILES: c1ccc(-c2nc(-c3ccc(-c4ncccn4)cc3)cc(-c3cc(-n4c5ccccc5c5ccccc54)cc(-n4c5ccccc5c5ccccc54)c3)n2)cc1]

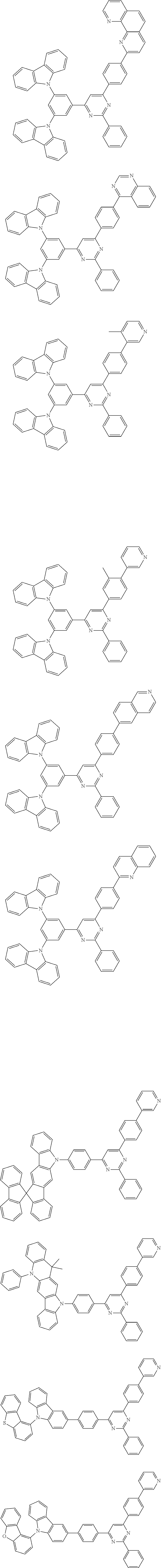

[chemical structure]

[chemical structure]

[chemical structure]

[chemical structure]

[chemical structure]

[chemical structure]

[chemical structure]

[chemical structure]

[chemical structure]

[chemical structure]

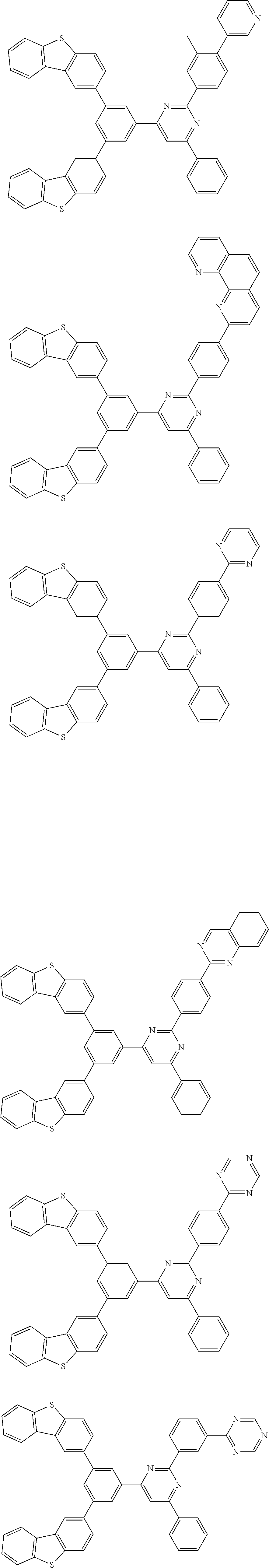

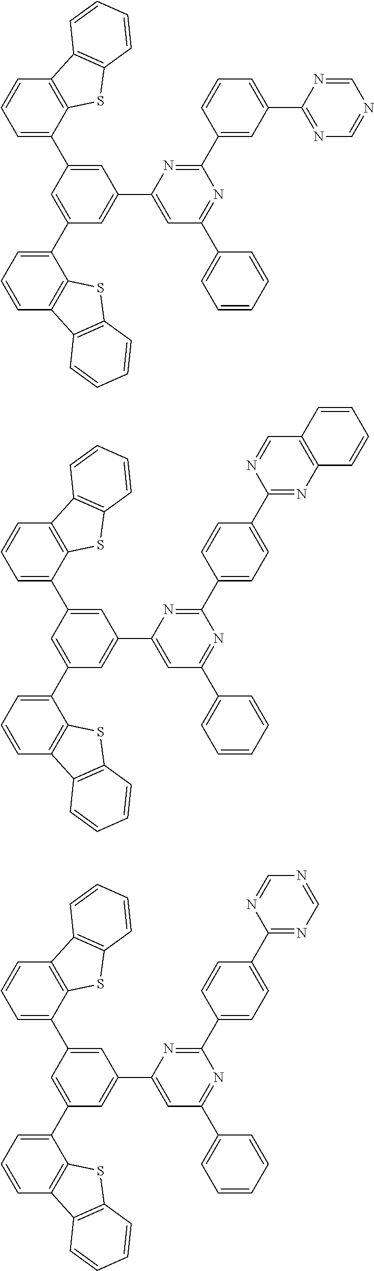 [SMILES: c1ccc(-c2cc(-c3cc(-c4cccc5c4sc4ccccc45)cc(-c4cccc5c4sc4ccccc45)c3)nc(-c3cccc(-c4ncncn4)c3)n2)cc1 ; c1ccc(-c2cc(-c3cc(-c4cccc5c4sc4ccccc45)cc(-c4cccc5c4sc4ccccc45)c3)nc(-c3ccc(-c4ncc5ccccc5n4)cc3)n2)cc1 ; c1ccc(-c2cc(-c3cc(-c4cccc5c4sc4ccccc45)cc(-c4cccc5c4sc4ccccc45)c3)nc(-c3ccc(-c4ncncn4)cc3)n2)cc1]

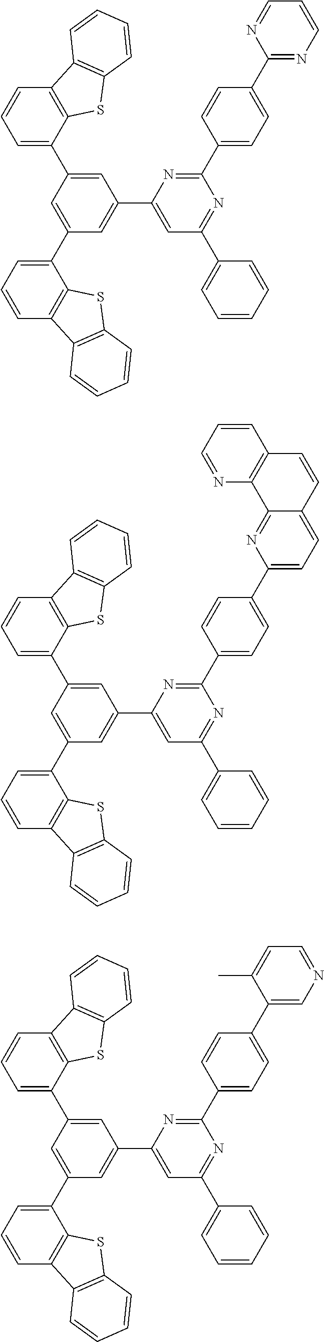 [SMILES: c1ccc(-c2cc(-c3cc(-c4cccc5c4sc4ccccc45)cc(-c4cccc5c4sc4ccccc45)c3)nc(-c3ccc(-c4ncccn4)cc3)n2)cc1 ; c1ccc(-c2cc(-c3cc(-c4cccc5c4sc4ccccc45)cc(-c4cccc5c4sc4ccccc45)c3)nc(-c3ccc(-c4ccc5ccc6cccnc6c5n4)cc3)n2)cc1 ; Cc1ccncc1-c1ccc(-c2nc(-c3ccccc3)cc(-c3cc(-c4cccc5c4sc4ccccc45)cc(-c4cccc5c4sc4ccccc45)c3)n2)cc1]

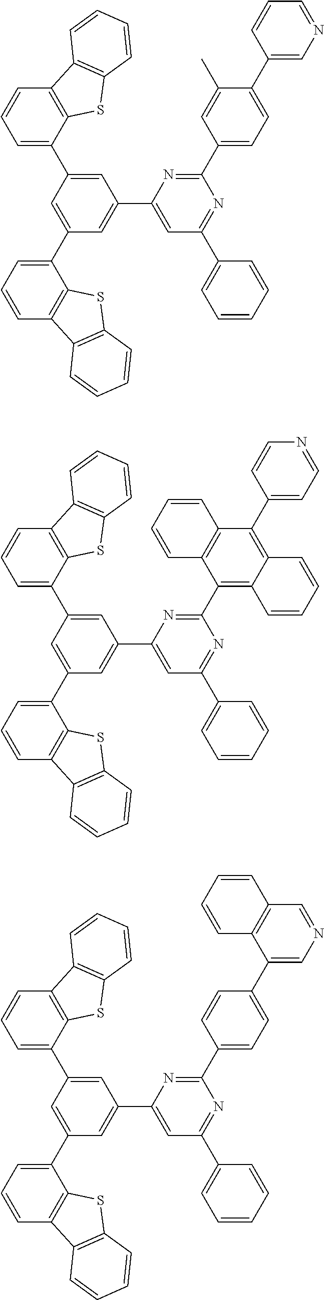 [SMILES: Cc1cc(-c2nc(-c3ccccc3)cc(-c3cc(-c4cccc5c4sc4ccccc45)cc(-c4cccc5c4sc4ccccc45)c3)n2)ccc1-c1cccnc1 ; c1ccc(-c2cc(-c3cc(-c4cccc5c4sc4ccccc45)cc(-c4cccc5c4sc4ccccc45)c3)nc(-c3c4ccccc4c(-c4ccncc4)c4ccccc34)n2)cc1 ; c1ccc(-c2cc(-c3cc(-c4cccc5c4sc4ccccc45)cc(-c4cccc5c4sc4ccccc45)c3)nc(-c3ccc(-c4cncc5ccccc45)cc3)n2)cc1]

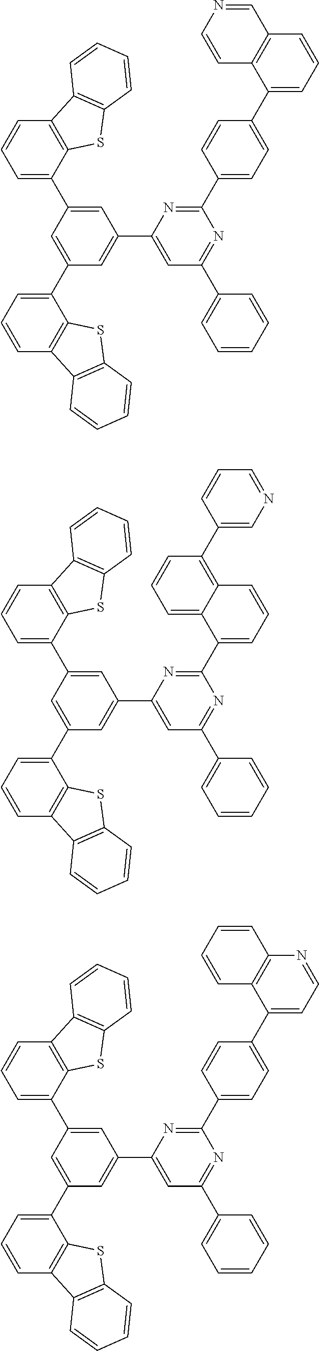 [SMILES: c1ccc(-c2cc(-c3cc(-c4cccc5c4sc4ccccc45)cc(-c4cccc5c4sc4ccccc45)c3)nc(-c3ccc(-c4cccc5cnccc45)cc3)n2)cc1 ; c1ccc(-c2cc(-c3cc(-c4cccc5c4sc4ccccc45)cc(-c4cccc5c4sc4ccccc45)c3)nc(-c3cccc4c(-c5cccnc5)cccc34)n2)cc1 ; c1ccc(-c2cc(-c3cc(-c4cccc5c4sc4ccccc45)cc(-c4cccc5c4sc4ccccc45)c3)nc(-c3ccc(-c4ccnc5ccccc45)cc3)n2)cc1]

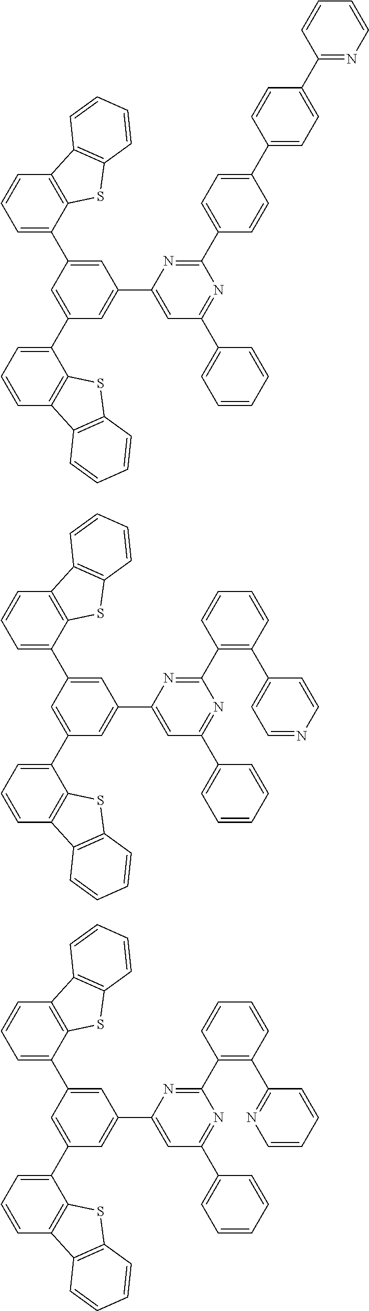

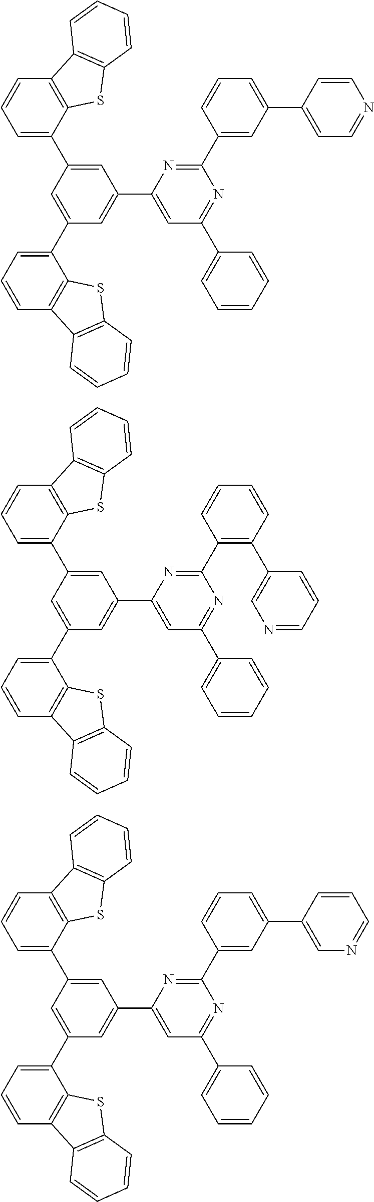

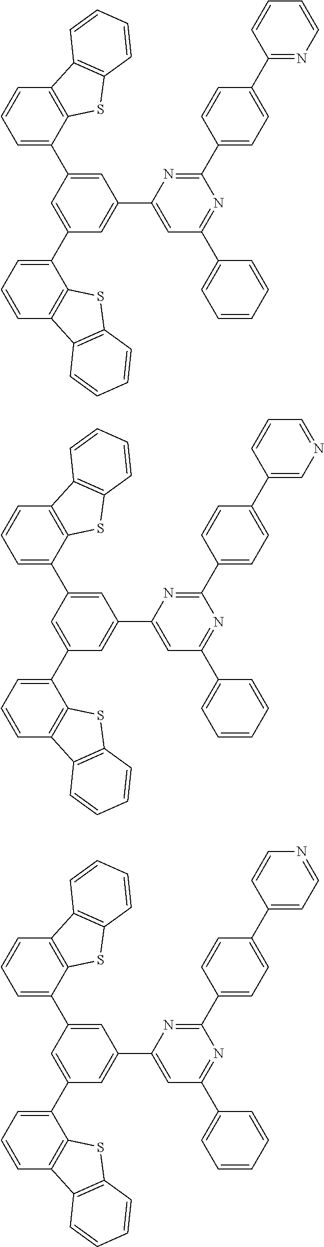

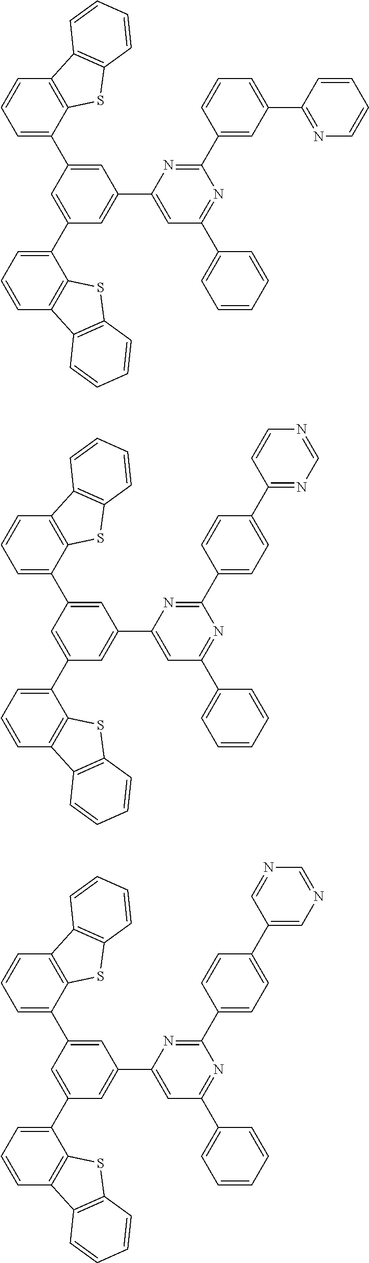 [SMILES: c1ccc(-c2cc(-c3cc(-c4cccc5c4sc4ccccc45)cc(-c4cccc5c4sc4ccccc45)c3)nc(-c3cccc(-c4ccccn4)c3)n2)cc1 ; c1ccc(-c2cc(-c3cc(-c4cccc5c4sc4ccccc45)cc(-c4cccc5c4sc4ccccc45)c3)nc(-c3ccc(-c4ccncn4)cc3)n2)cc1 ; c1ccc(-c2cc(-c3cc(-c4cccc5c4sc4ccccc45)cc(-c4cccc5c4sc4ccccc45)c3)nc(-c3ccc(-c4cncnc4)cc3)n2)cc1]

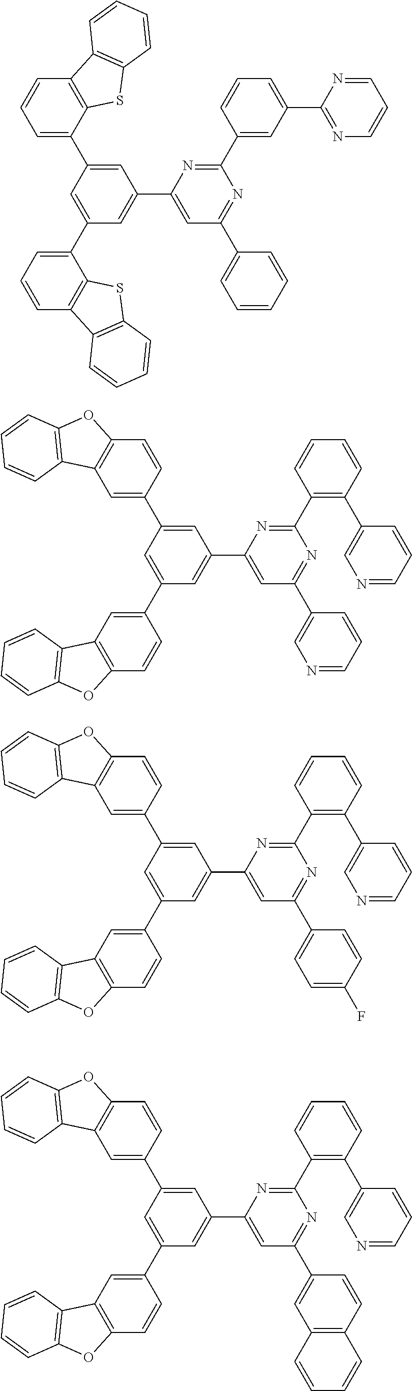

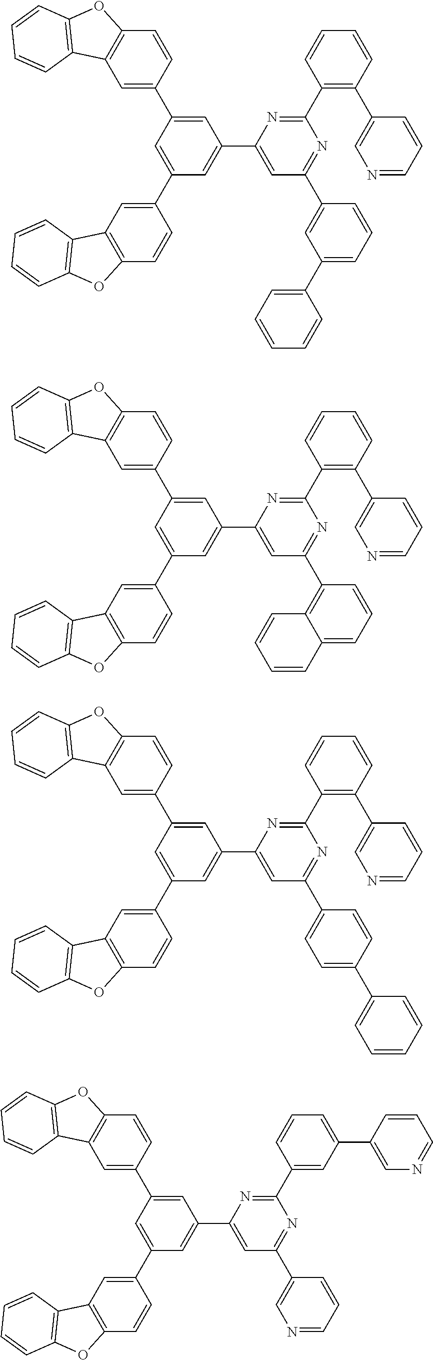

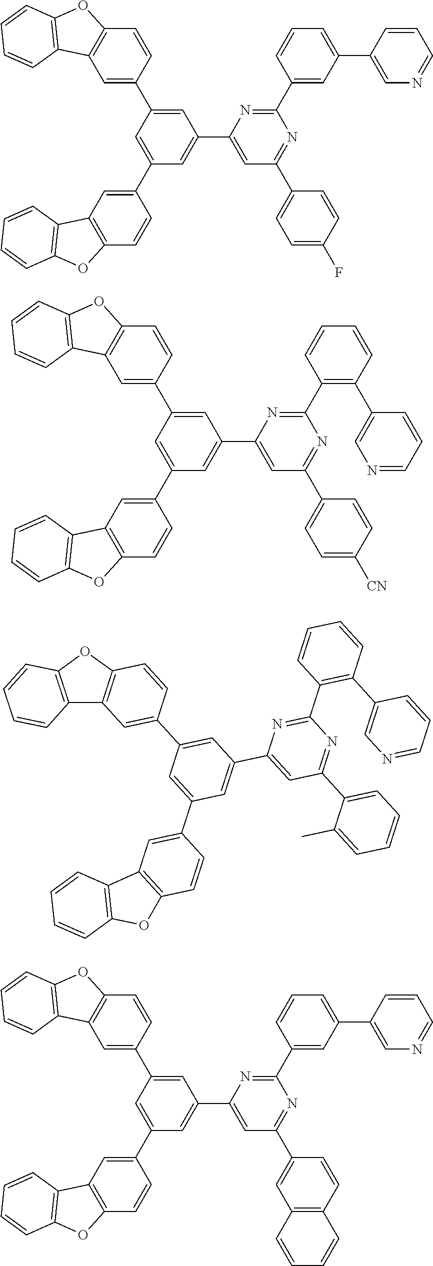

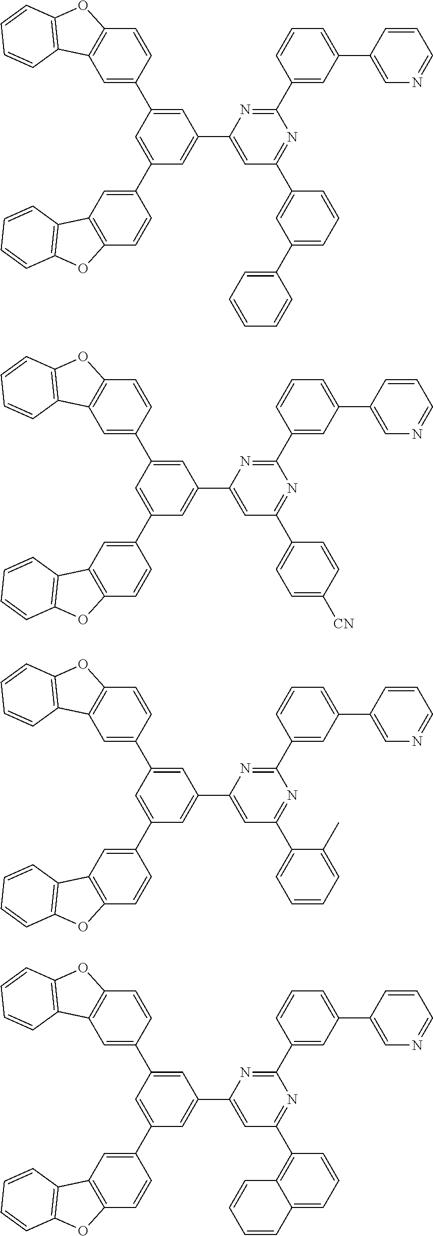

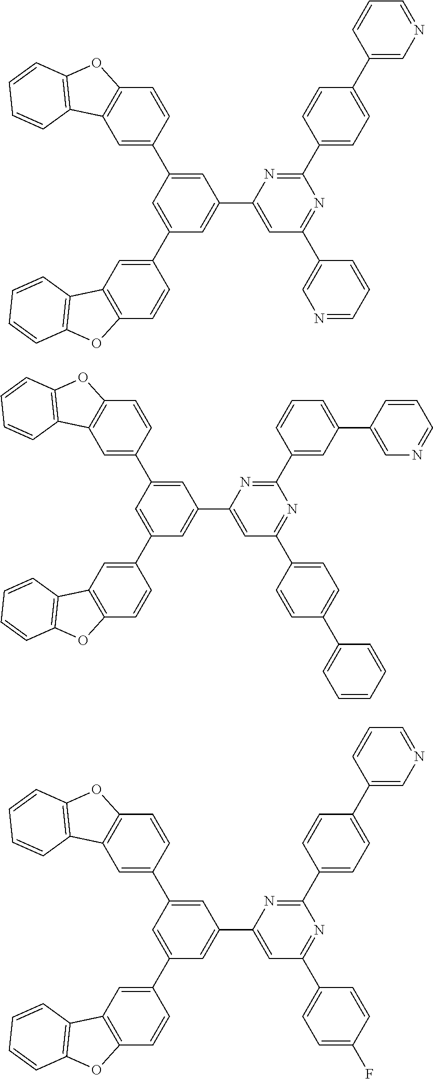

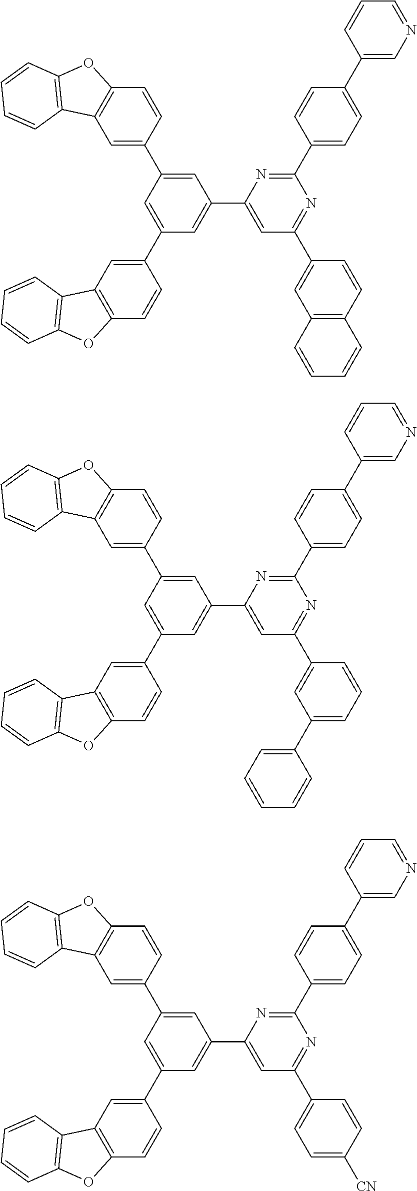

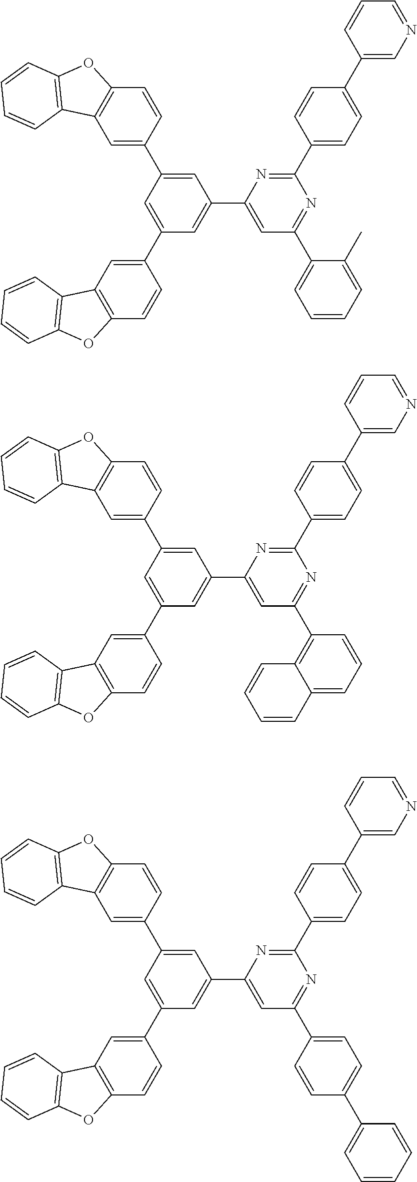 [SMILES: Cc1ccccc1-c1cc(-c2cc(-c3ccc4oc5ccccc5c4c3)cc(-c3ccc4oc5ccccc5c4c3)c2)nc(-c2ccc(-c3cccnc3)cc2)n1 ; c1cncc(-c2ccc(-c3nc(-c4cc(-c5ccc6oc7ccccc7c6c5)cc(-c5ccc6oc7ccccc7c6c5)c4)cc(-c4cccc5ccccc45)n3)cc2)c1 ; c1ccc(-c2ccc(-c3cc(-c4cc(-c5ccc6oc7ccccc7c6c5)cc(-c5ccc6oc7ccccc7c6c5)c4)nc(-c4ccc(-c5cccnc5)cc4)n3)cc2)cc1]

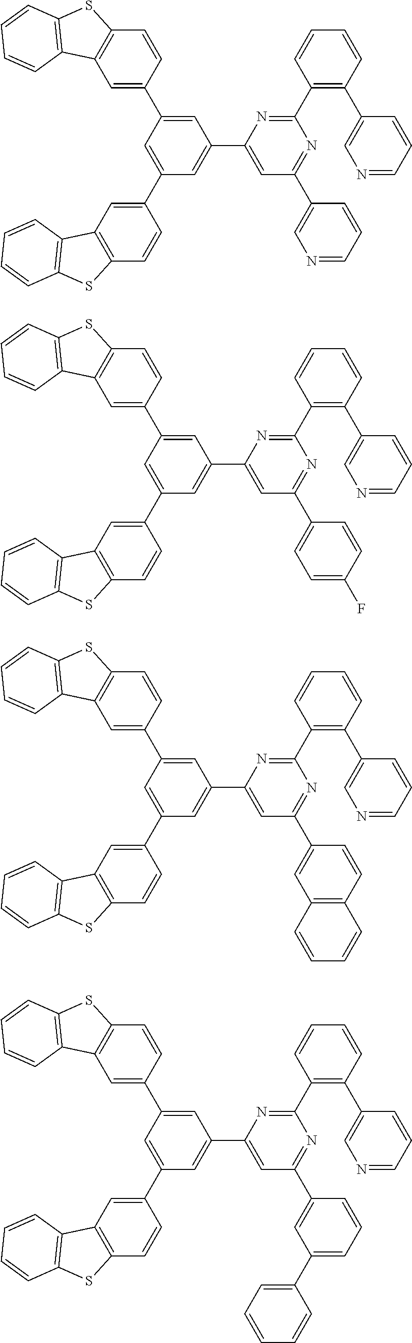

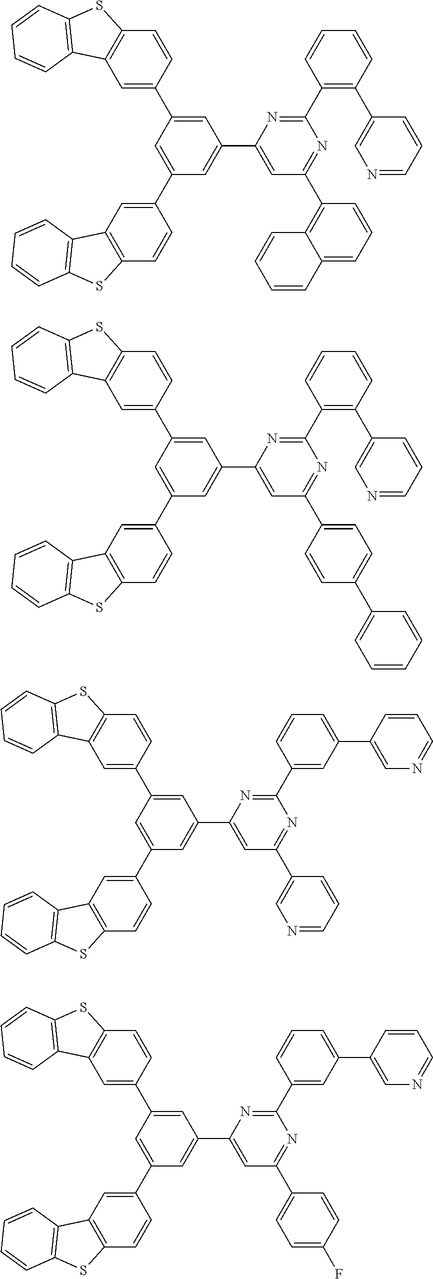

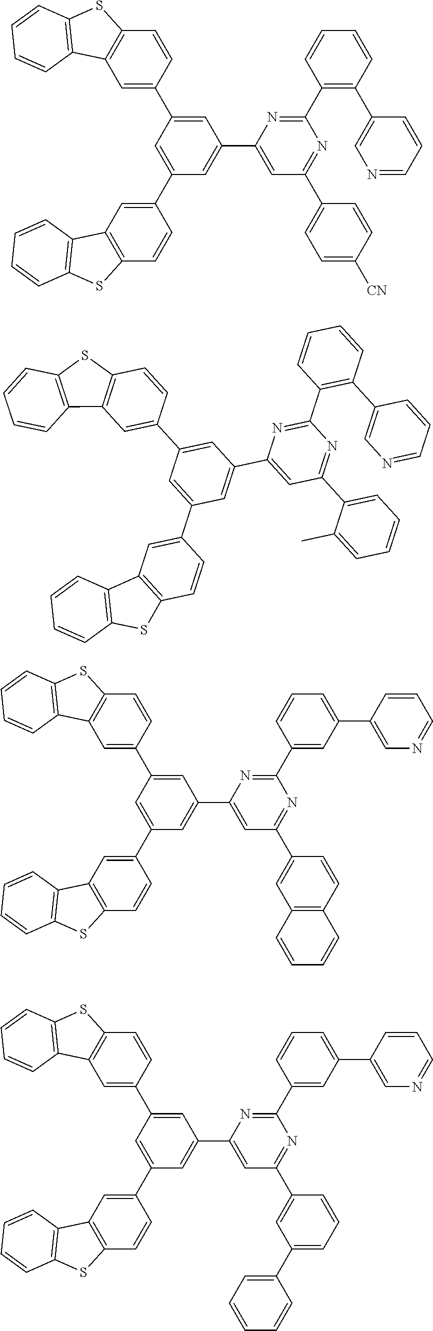

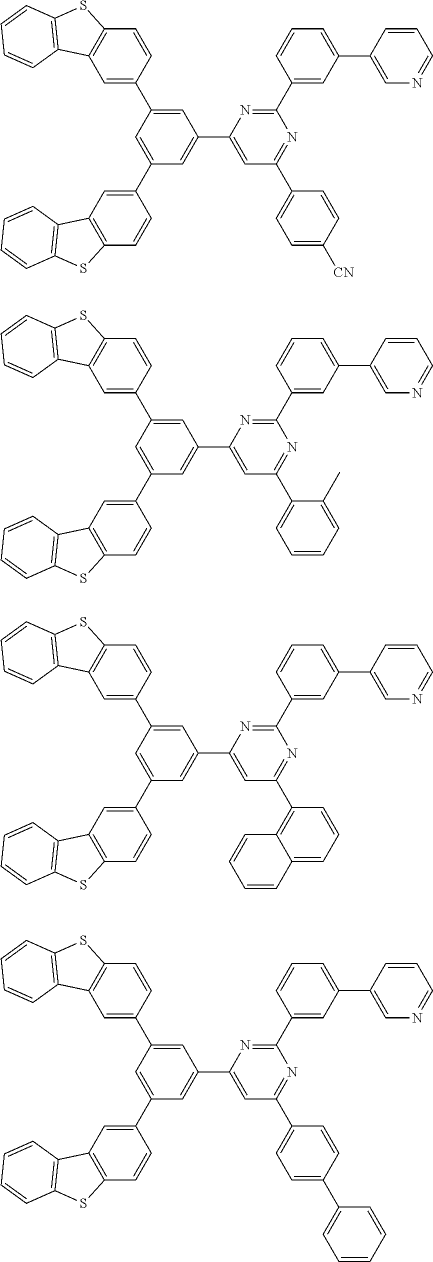

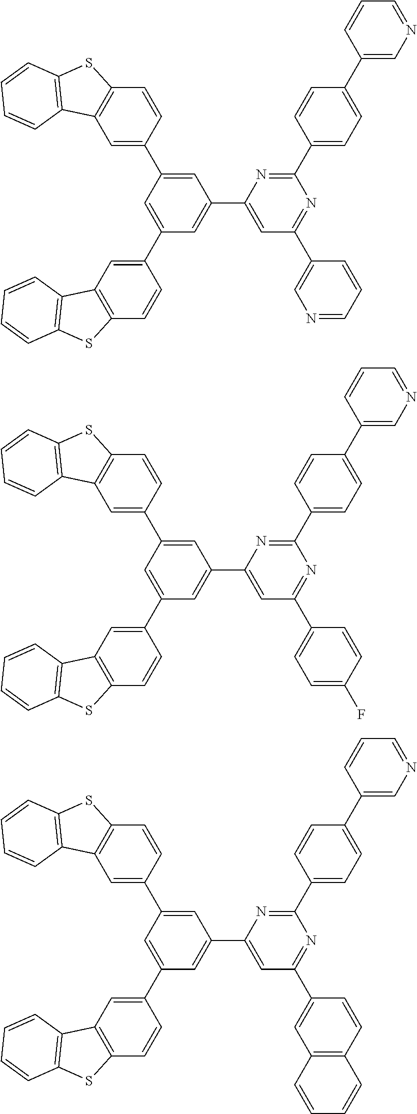

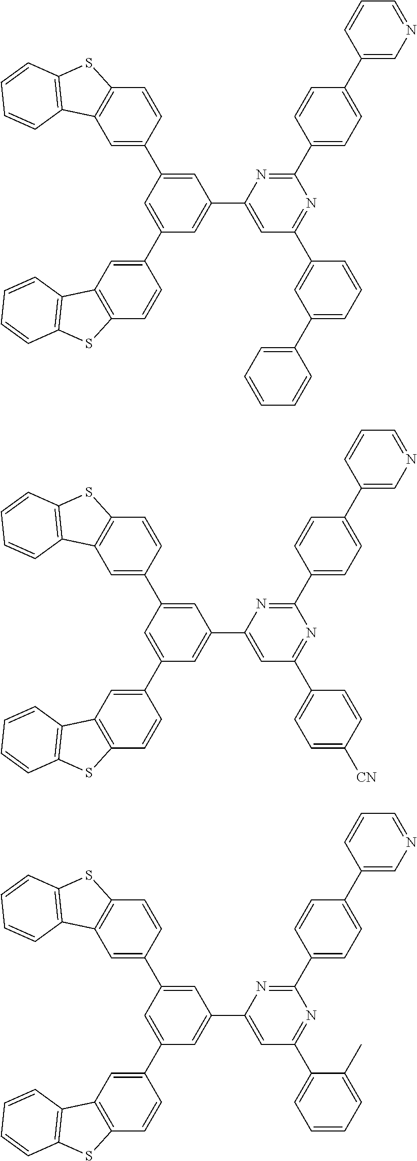

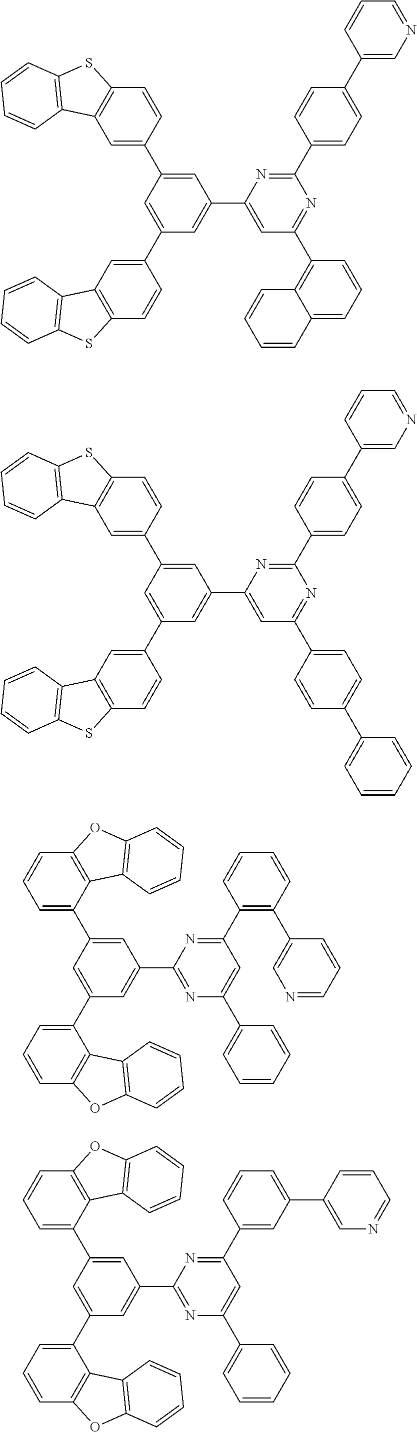

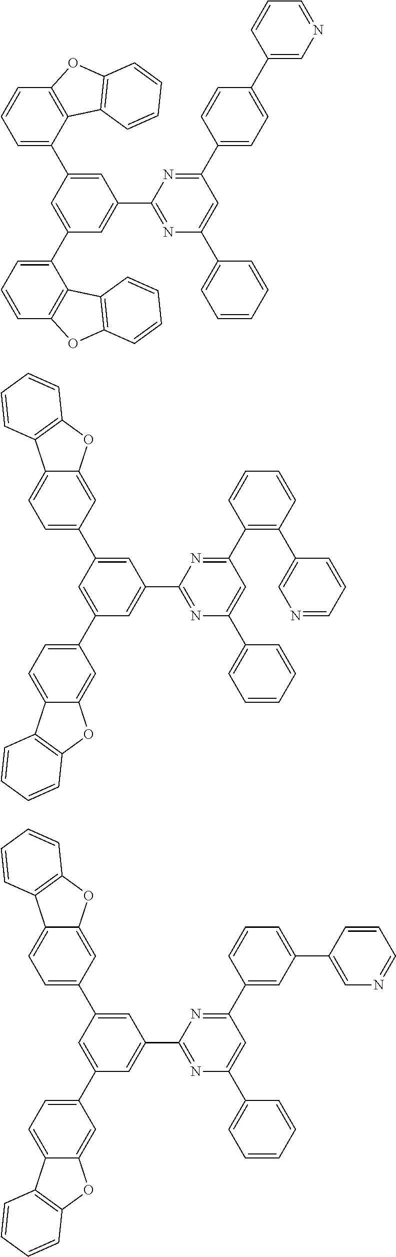

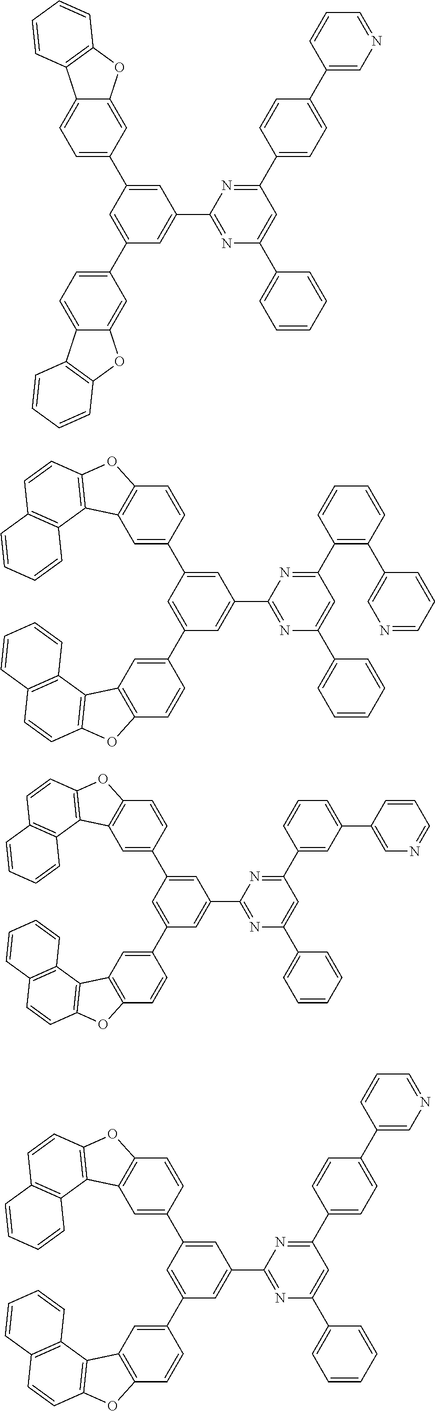

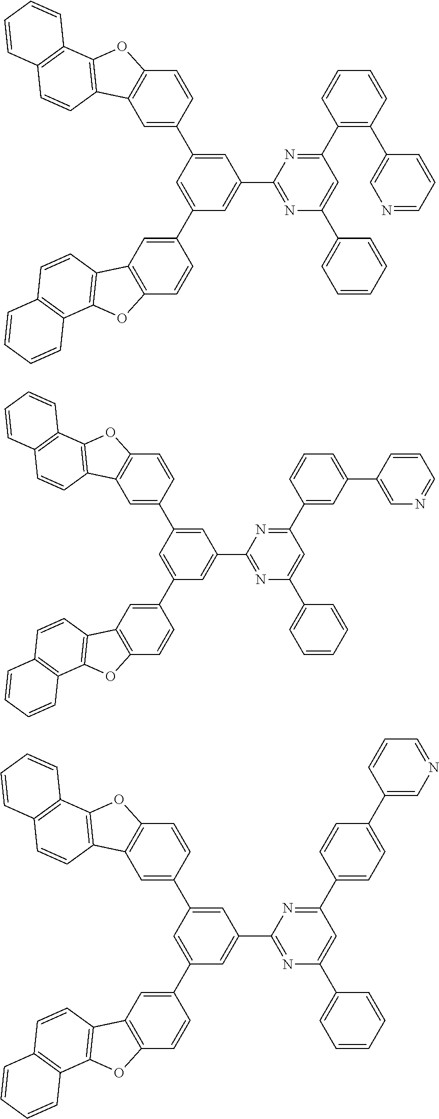

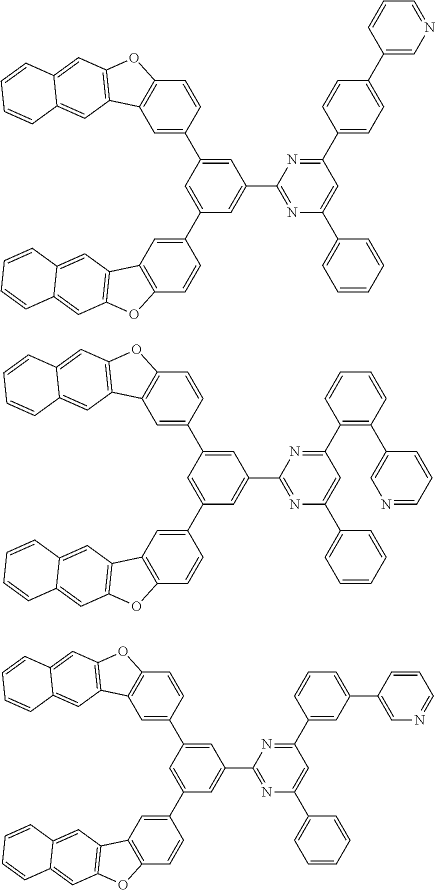

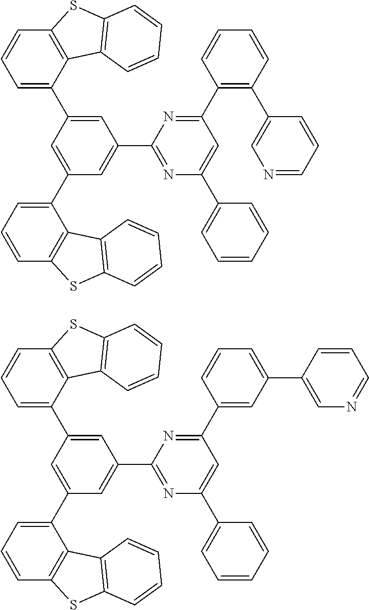

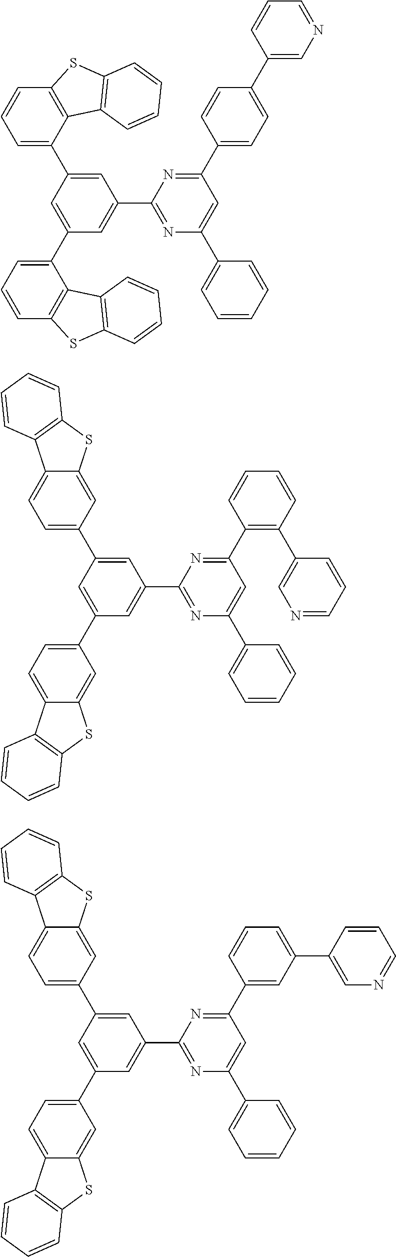

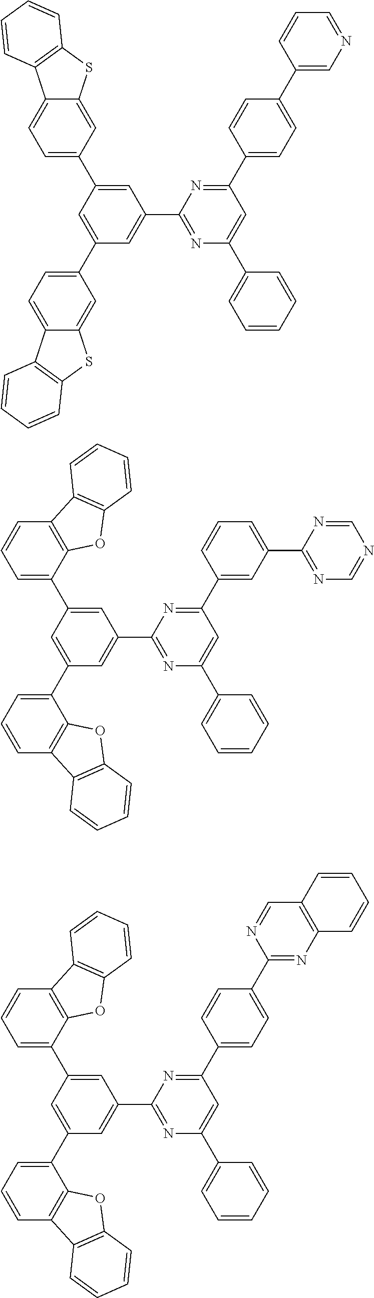 [SMILES: c1ccc(-c2cc(-c3ccc(-c4cccnc4)cc3)nc(-c3cc(-c4ccc5c(c4)sc4ccccc45)cc(-c4ccc5c(c4)sc4ccccc45)c3)n2)cc1 ; c1ccc(-c2cc(-c3cccc(-c4ncncn4)c3)nc(-c3cc(-c4cccc5c4oc4ccccc45)cc(-c4cccc5c4oc4ccccc45)c3)n2)cc1 ; c1ccc(-c2cc(-c3ccc(-c4ncc5ccccc5n4)cc3)nc(-c3cc(-c4cccc5c4oc4ccccc45)cc(-c4cccc5c4oc4ccccc45)c3)n2)cc1]

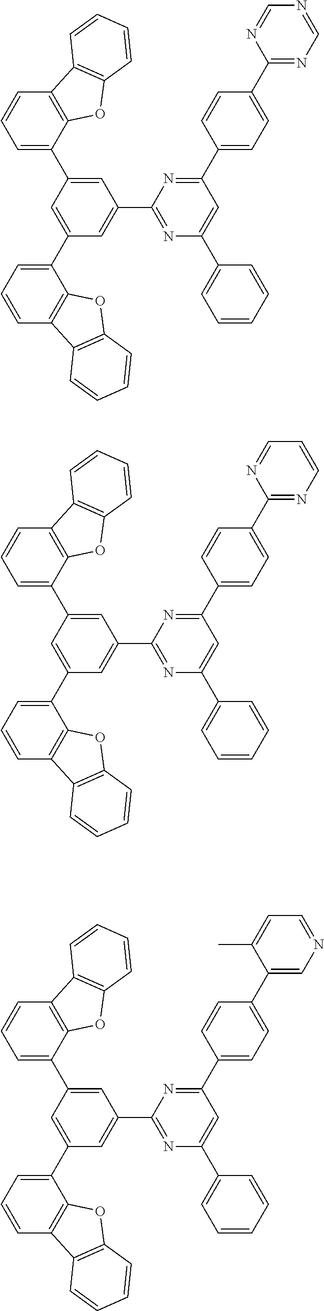 [SMILES: c1ccc(-c2cc(-c3ccc(-c4ncncn4)cc3)nc(-c3cc(-c4cccc5c4oc4ccccc45)cc(-c4cccc5c4oc4ccccc45)c3)n2)cc1 ; c1ccc(-c2cc(-c3ccc(-c4ncccn4)cc3)nc(-c3cc(-c4cccc5c4oc4ccccc45)cc(-c4cccc5c4oc4ccccc45)c3)n2)cc1 ; Cc1ccncc1-c1ccc(-c2cc(-c3ccccc3)nc(-c3cc(-c4cccc5c4oc4ccccc45)cc(-c4cccc5c4oc4ccccc45)c3)n2)cc1]

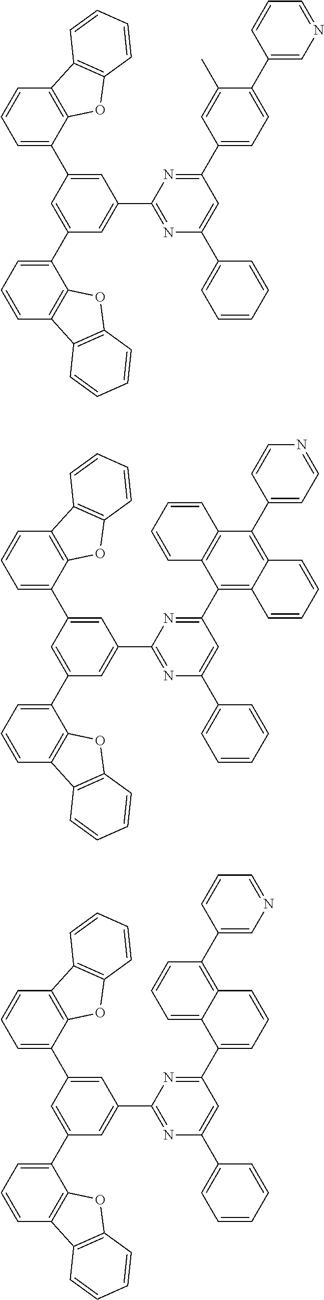 [SMILES: Cc1cc(-c2cc(-c3ccccc3)nc(-c3cc(-c4cccc5c4oc4ccccc45)cc(-c4cccc5c4oc4ccccc45)c3)n2)ccc1-c1cccnc1 ; c1ccc(-c2cc(-c3c4ccccc4c(-c4ccncc4)c4ccccc34)nc(-c3cc(-c4cccc5c4oc4ccccc45)cc(-c4cccc5c4oc4ccccc45)c3)n2)cc1 ; c1ccc(-c2cc(-c3cccc4c(-c5cccnc5)cccc34)nc(-c3cc(-c4cccc5c4oc4ccccc45)cc(-c4cccc5c4oc4ccccc45)c3)n2)cc1]

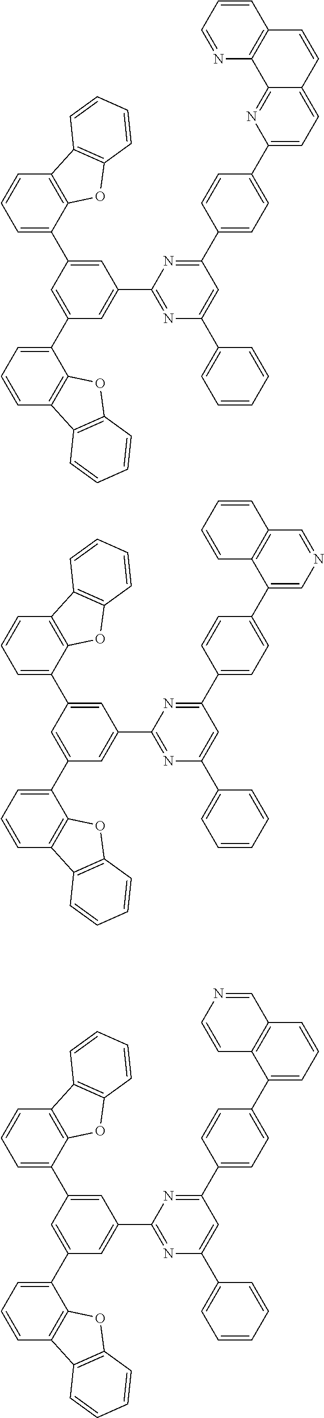 [SMILES: c1ccc(-c2cc(-c3ccc(-c4ccc5ccc6cccnc6c5n4)cc3)nc(-c3cc(-c4cccc5c4oc4ccccc45)cc(-c4cccc5c4oc4ccccc45)c3)n2)cc1 ; c1ccc(-c2cc(-c3ccc(-c4cncc5ccccc45)cc3)nc(-c3cc(-c4cccc5c4oc4ccccc45)cc(-c4cccc5c4oc4ccccc45)c3)n2)cc1 ; c1ccc(-c2cc(-c3ccc(-c4cccc5cnccc45)cc3)nc(-c3cc(-c4cccc5c4oc4ccccc45)cc(-c4cccc5c4oc4ccccc45)c3)n2)cc1]

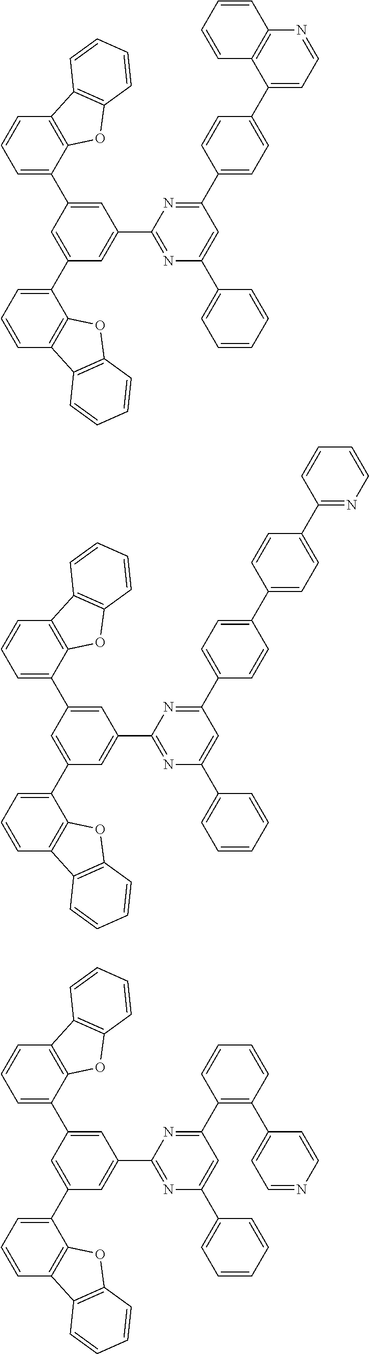

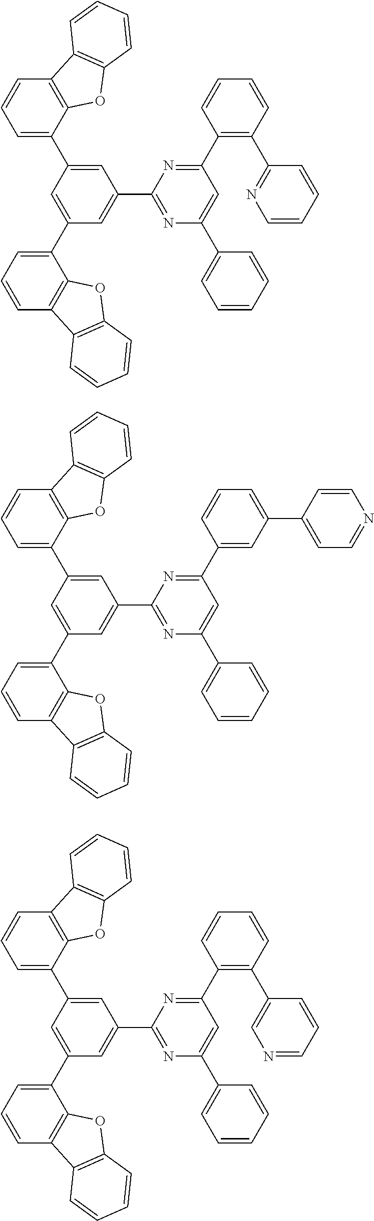

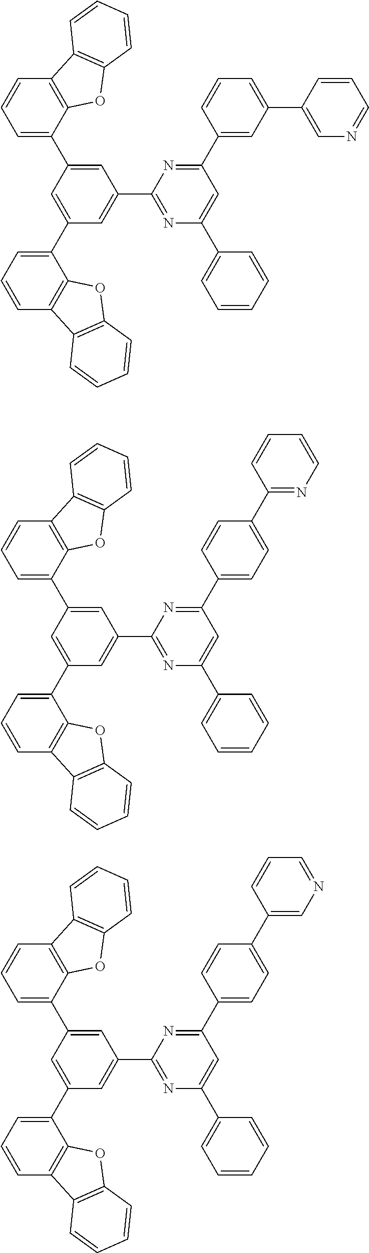

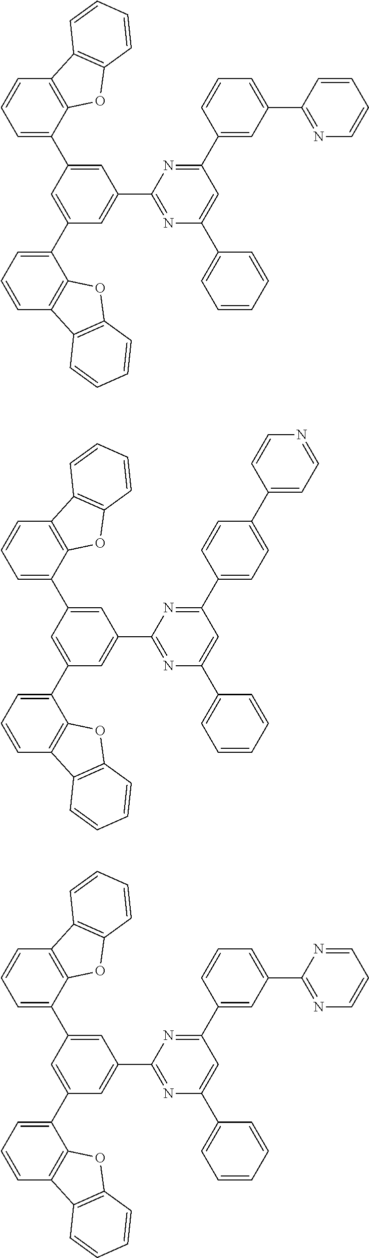

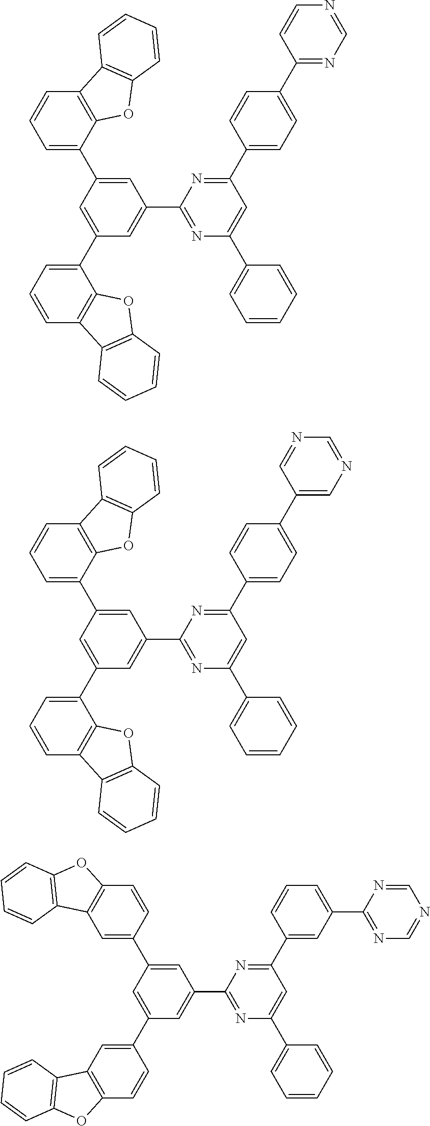

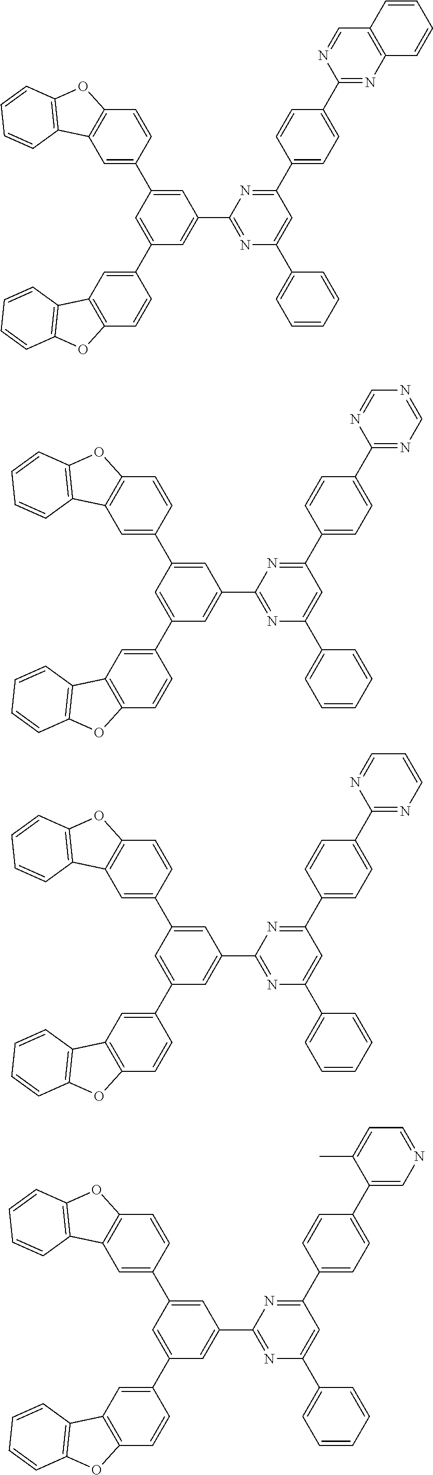 [SMILES: c1ccc(-c2cc(-c3ccc(-c4ncc5ccccc5n4)cc3)nc(-c3cc(-c4ccc5oc6ccccc6c5c4)cc(-c4ccc5oc6ccccc6c5c4)c3)n2)cc1 ; c1ccc(-c2cc(-c3ccc(-c4ncncn4)cc3)nc(-c3cc(-c4ccc5oc6ccccc6c5c4)cc(-c4ccc5oc6ccccc6c5c4)c3)n2)cc1 ; c1ccc(-c2cc(-c3ccc(-c4ncccn4)cc3)nc(-c3cc(-c4ccc5oc6ccccc6c5c4)cc(-c4ccc5oc6ccccc6c5c4)c3)n2)cc1 ; Cc1ccncc1-c1ccc(-c2cc(-c3ccccc3)nc(-c3cc(-c4ccc5oc6ccccc6c5c4)cc(-c4ccc5oc6ccccc6c5c4)c3)n2)cc1]

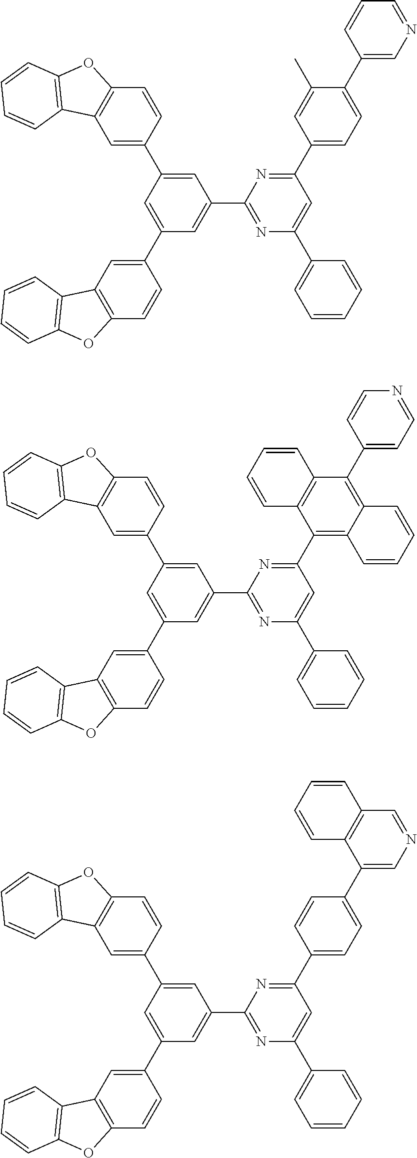 [SMILES: Cc1cc(-c2cc(-c3ccccc3)nc(-c3cc(-c4ccc5oc6ccccc6c5c4)cc(-c4ccc5oc6ccccc6c5c4)c3)n2)ccc1-c1cccnc1 ; c1ccc(-c2cc(-c3c4ccccc4c(-c4ccncc4)c4ccccc34)nc(-c3cc(-c4ccc5oc6ccccc6c5c4)cc(-c4ccc5oc6ccccc6c5c4)c3)n2)cc1 ; c1ccc(-c2cc(-c3ccc(-c4cncc5ccccc45)cc3)nc(-c3cc(-c4ccc5oc6ccccc6c5c4)cc(-c4ccc5oc6ccccc6c5c4)c3)n2)cc1]

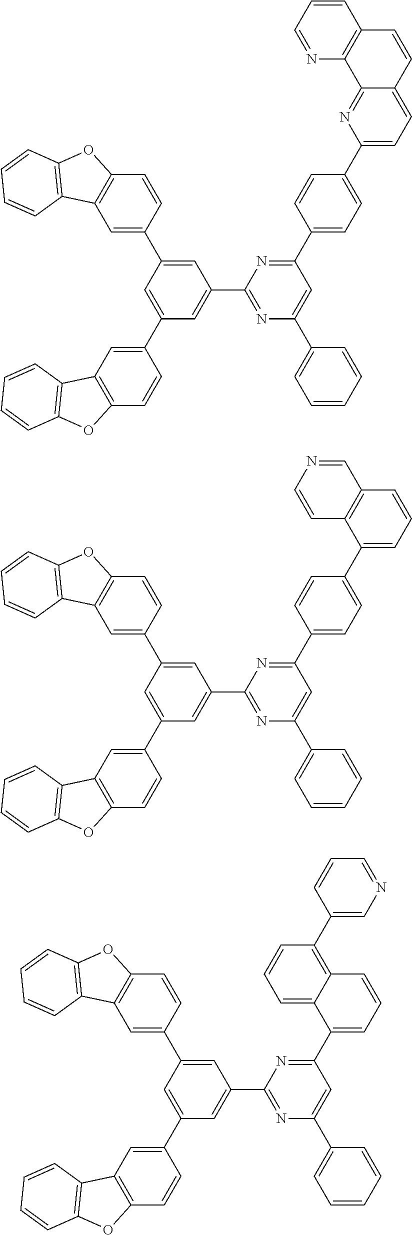 [SMILES: c1ccc(-c2cc(-c3ccc(-c4ccc5ccc6cccnc6c5n4)cc3)nc(-c3cc(-c4ccc5oc6ccccc6c5c4)cc(-c4ccc5oc6ccccc6c5c4)c3)n2)cc1 ; c1ccc(-c2cc(-c3ccc(-c4cccc5cnccc45)cc3)nc(-c3cc(-c4ccc5oc6ccccc6c5c4)cc(-c4ccc5oc6ccccc6c5c4)c3)n2)cc1 ; c1ccc(-c2cc(-c3cccc4c(-c5cccnc5)cccc34)nc(-c3cc(-c4ccc5oc6ccccc6c5c4)cc(-c4ccc5oc6ccccc6c5c4)c3)n2)cc1]

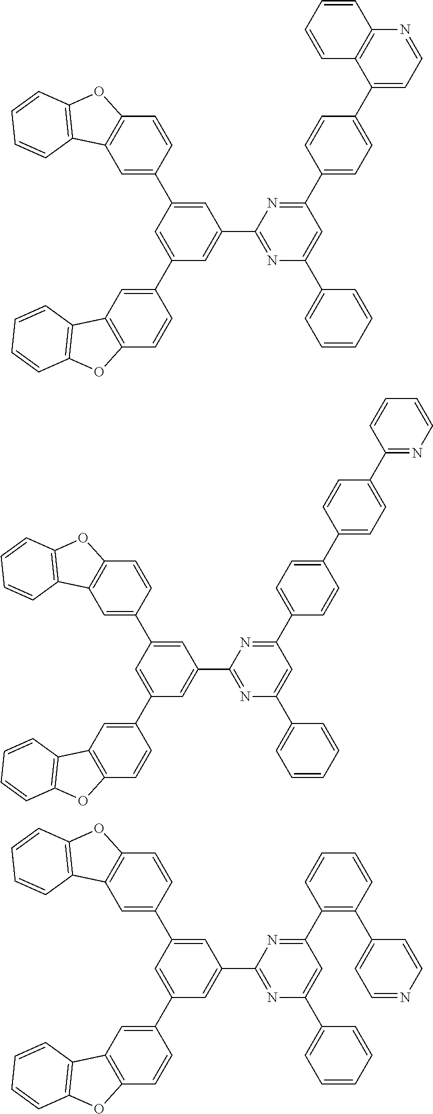

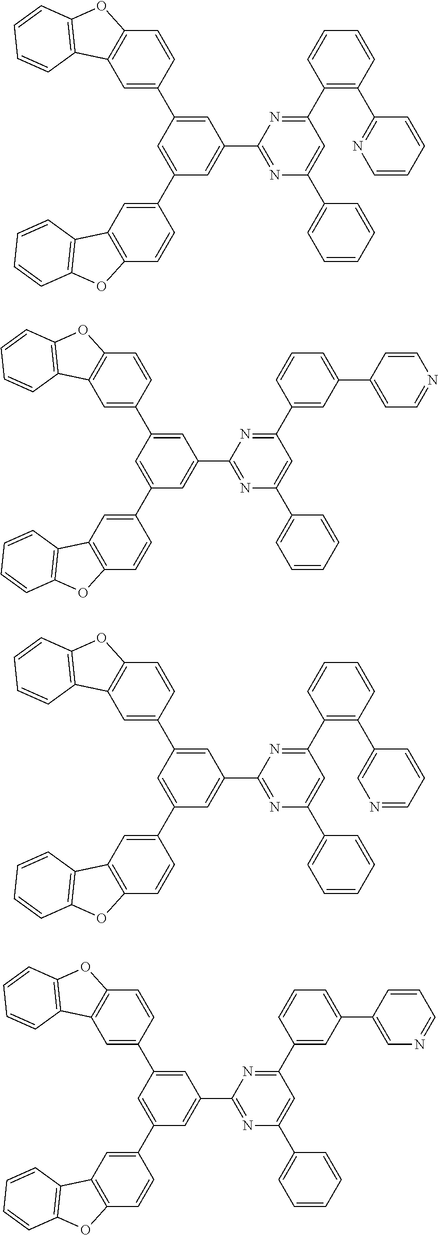 [SMILES: c1ccc(-c2cc(-c3ccccc3-c3ccccn3)nc(-c3cc(-c4ccc5oc6ccccc6c5c4)cc(-c4ccc5oc6ccccc6c5c4)c3)n2)cc1 ; c1ccc(-c2cc(-c3cccc(-c4ccncc4)c3)nc(-c3cc(-c4ccc5oc6ccccc6c5c4)cc(-c4ccc5oc6ccccc6c5c4)c3)n2)cc1 ; c1ccc(-c2cc(-c3ccccc3-c3cccnc3)nc(-c3cc(-c4ccc5oc6ccccc6c5c4)cc(-c4ccc5oc6ccccc6c5c4)c3)n2)cc1 ; c1ccc(-c2cc(-c3cccc(-c4cccnc4)c3)nc(-c3cc(-c4ccc5oc6ccccc6c5c4)cc(-c4ccc5oc6ccccc6c5c4)c3)n2)cc1]

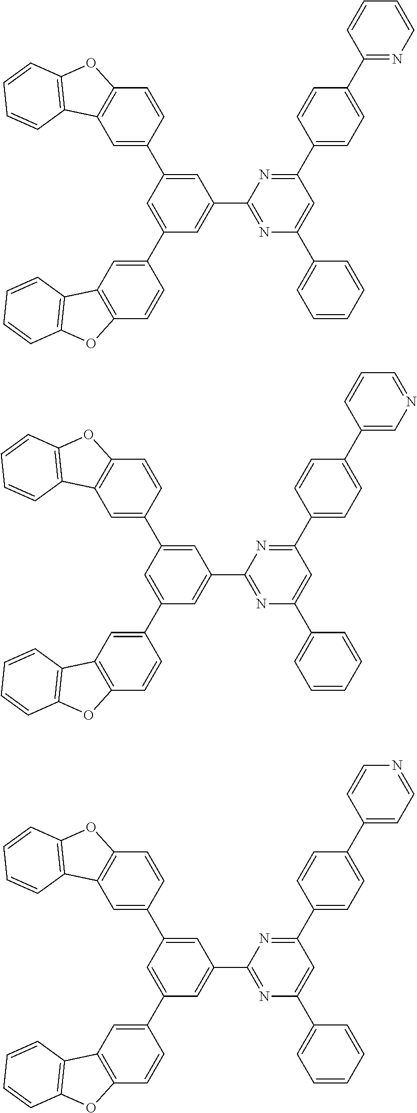 [SMILES: c1ccc(-c2cc(-c3ccc(-c4ccccn4)cc3)nc(-c3cc(-c4ccc5oc6ccccc6c5c4)cc(-c4ccc5oc6ccccc6c5c4)c3)n2)cc1 ; c1ccc(-c2cc(-c3ccc(-c4cccnc4)cc3)nc(-c3cc(-c4ccc5oc6ccccc6c5c4)cc(-c4ccc5oc6ccccc6c5c4)c3)n2)cc1 ; c1ccc(-c2cc(-c3ccc(-c4ccncc4)cc3)nc(-c3cc(-c4ccc5oc6ccccc6c5c4)cc(-c4ccc5oc6ccccc6c5c4)c3)n2)cc1]

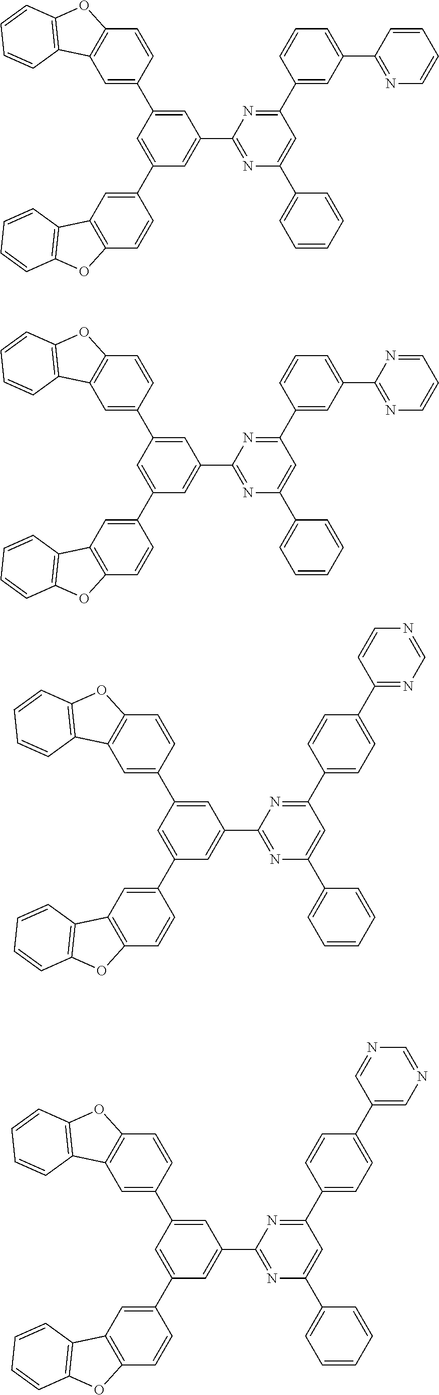 [SMILES: c1ccc(-c2cc(-c3cccc(-c4ccccn4)c3)nc(-c3cc(-c4ccc5oc6ccccc6c5c4)cc(-c4ccc5oc6ccccc6c5c4)c3)n2)cc1 ; c1ccc(-c2cc(-c3cccc(-c4ncccn4)c3)nc(-c3cc(-c4ccc5oc6ccccc6c5c4)cc(-c4ccc5oc6ccccc6c5c4)c3)n2)cc1 ; c1ccc(-c2cc(-c3ccc(-c4ccncn4)cc3)nc(-c3cc(-c4ccc5oc6ccccc6c5c4)cc(-c4ccc5oc6ccccc6c5c4)c3)n2)cc1 ; c1ccc(-c2cc(-c3ccc(-c4cncnc4)cc3)nc(-c3cc(-c4ccc5oc6ccccc6c5c4)cc(-c4ccc5oc6ccccc6c5c4)c3)n2)cc1]

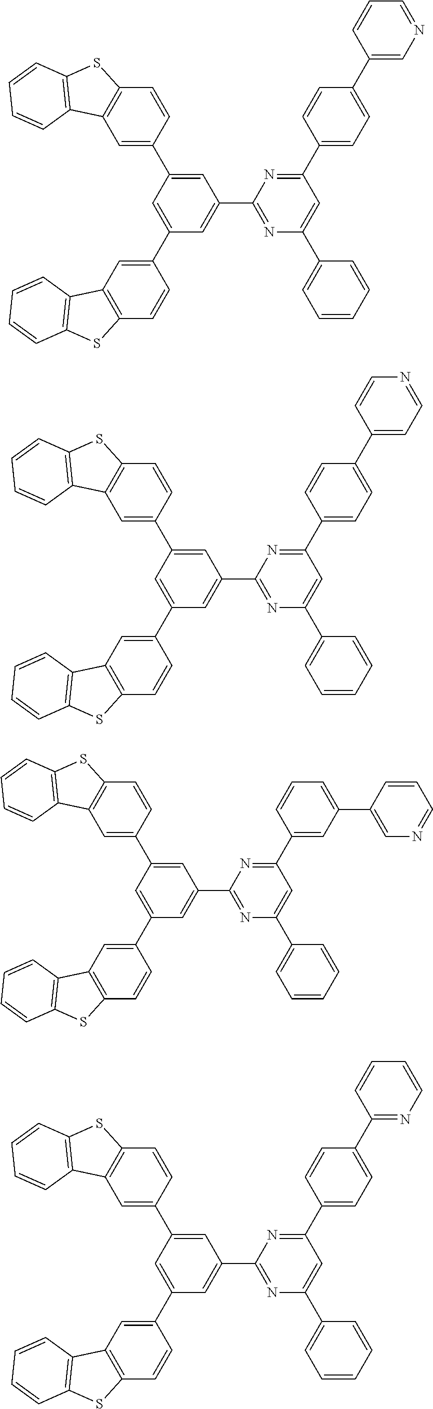

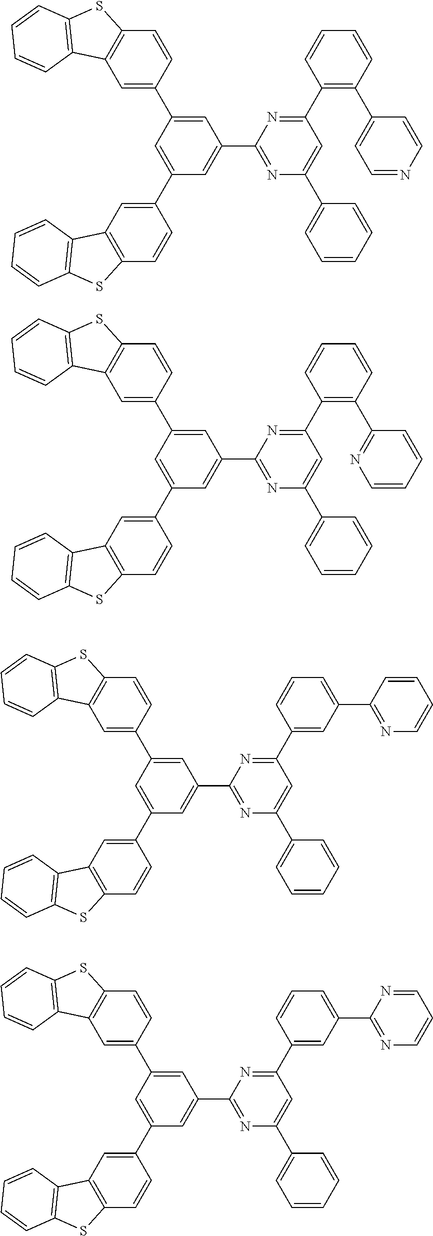

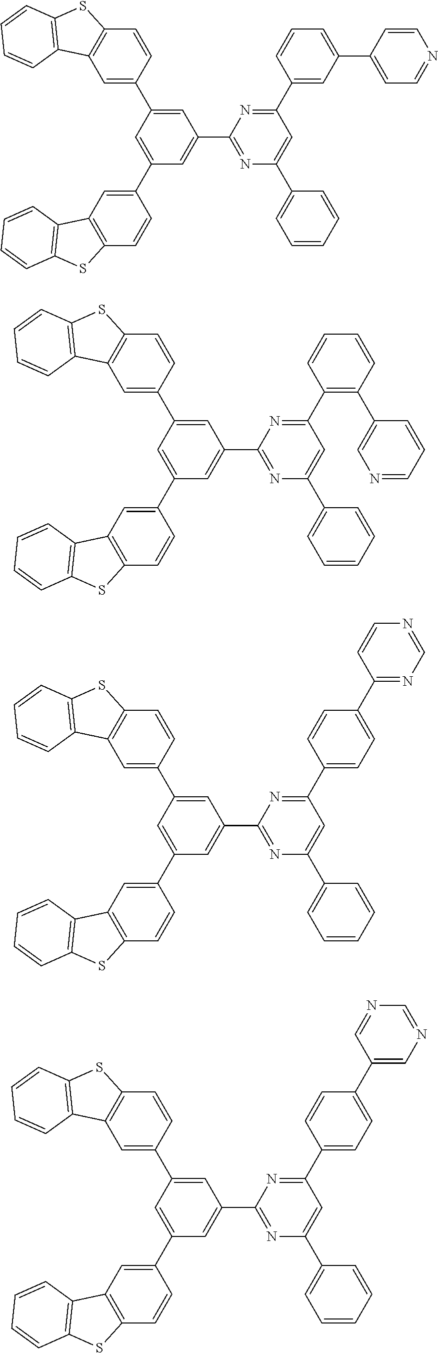

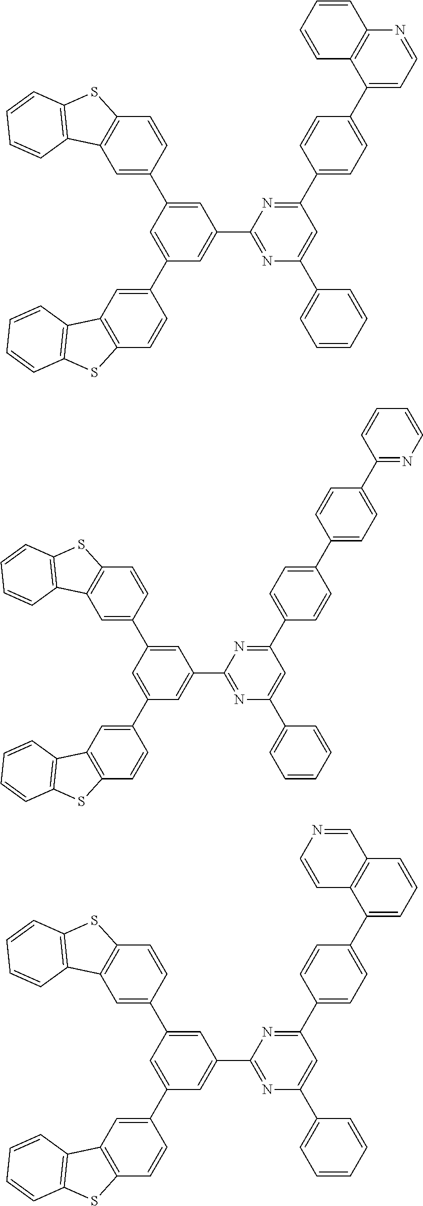 [SMILES: c1ccc(-c2cc(-c3ccc(-c4ccnc5ccccc45)cc3)nc(-c3cc(-c4ccc5sc6ccccc6c5c4)cc(-c4ccc5sc6ccccc6c5c4)c3)n2)cc1 ; c1ccc(-c2cc(-c3ccc(-c4ccc(-c5ccccn5)cc4)cc3)nc(-c3cc(-c4ccc5sc6ccccc6c5c4)cc(-c4ccc5sc6ccccc6c5c4)c3)n2)cc1 ; c1ccc(-c2cc(-c3ccc(-c4cccc5cnccc45)cc3)nc(-c3cc(-c4ccc5sc6ccccc6c5c4)cc(-c4ccc5sc6ccccc6c5c4)c3)n2)cc1]

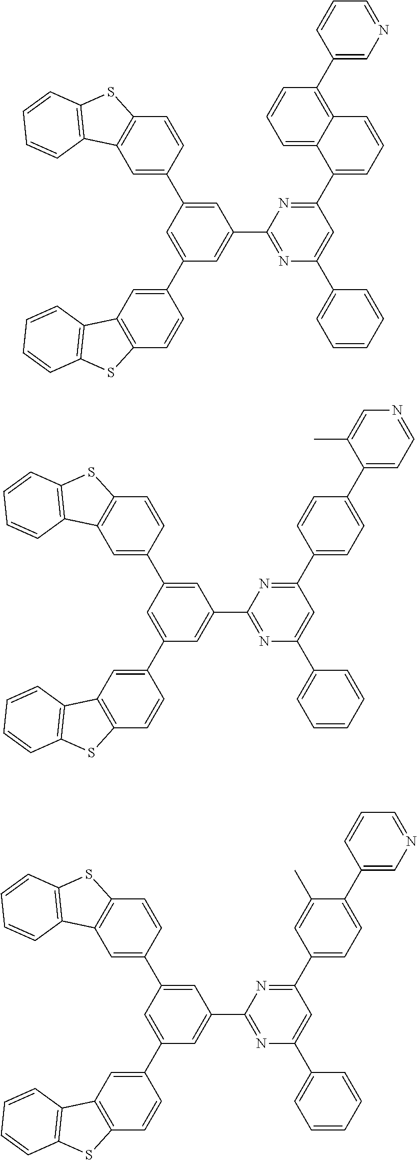 [SMILES: c1ccc(-c2cc(-c3cccc4c(-c5cccnc5)cccc34)nc(-c3cc(-c4ccc5sc6ccccc6c5c4)cc(-c4ccc5sc6ccccc6c5c4)c3)n2)cc1 ; Cc1cnccc1-c1ccc(-c2cc(-c3ccccc3)nc(-c3cc(-c4ccc5sc6ccccc6c5c4)cc(-c4ccc5sc6ccccc6c5c4)c3)n2)cc1 ; Cc1cc(-c2cc(-c3ccccc3)nc(-c3cc(-c4ccc5sc6ccccc6c5c4)cc(-c4ccc5sc6ccccc6c5c4)c3)n2)ccc1-c1cccnc1]

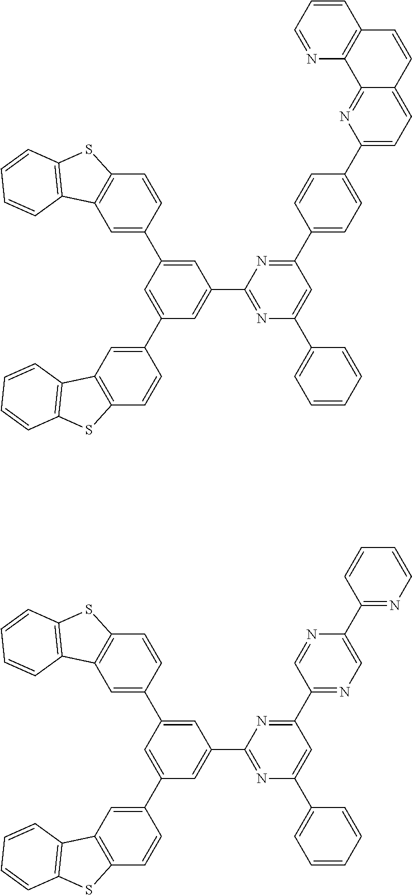 [SMILES: c1ccc(-c2cc(-c3ccc(-c4ccc5ccc6cccnc6c5n4)cc3)nc(-c3cc(-c4ccc5sc6ccccc6c5c4)cc(-c4ccc5sc6ccccc6c5c4)c3)n2)cc1 ; c1ccc(-c2cc(-c3cnc(-c4ccccn4)cn3)nc(-c3cc(-c4ccc5sc6ccccc6c5c4)cc(-c4ccc5sc6ccccc6c5c4)c3)n2)cc1]

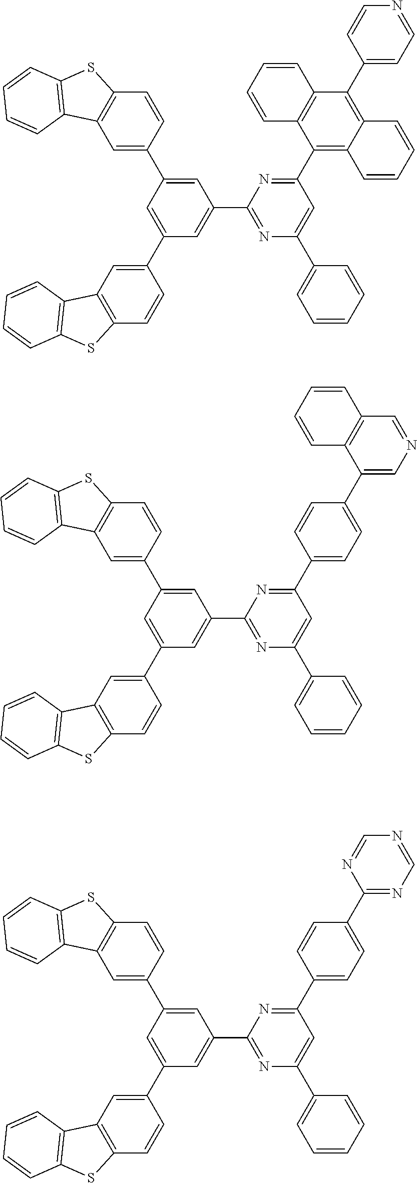 [SMILES: c1ccc(-c2cc(-c3c4ccccc4c(-c4ccncc4)c4ccccc34)nc(-c3cc(-c4ccc5sc6ccccc6c5c4)cc(-c4ccc5sc6ccccc6c5c4)c3)n2)cc1 ; c1ccc(-c2cc(-c3ccc(-c4cncc5ccccc45)cc3)nc(-c3cc(-c4ccc5sc6ccccc6c5c4)cc(-c4ccc5sc6ccccc6c5c4)c3)n2)cc1 ; c1ccc(-c2cc(-c3ccc(-c4ncncn4)cc3)nc(-c3cc(-c4ccc5sc6ccccc6c5c4)cc(-c4ccc5sc6ccccc6c5c4)c3)n2)cc1]

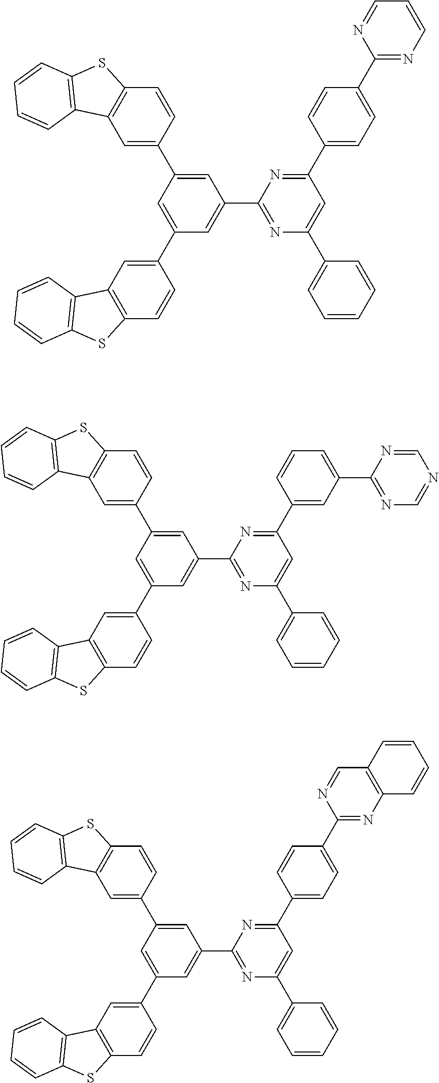 [SMILES: c1ccc(-c2cc(-c3ccc(-c4ncccn4)cc3)nc(-c3cc(-c4ccc5sc6ccccc6c5c4)cc(-c4ccc5sc6ccccc6c5c4)c3)n2)cc1 ; c1ccc(-c2cc(-c3cccc(-c4ncncn4)c3)nc(-c3cc(-c4ccc5sc6ccccc6c5c4)cc(-c4ccc5sc6ccccc6c5c4)c3)n2)cc1 ; c1ccc(-c2cc(-c3ccc(-c4ncc5ccccc5n4)cc3)nc(-c3cc(-c4ccc5sc6ccccc6c5c4)cc(-c4ccc5sc6ccccc6c5c4)c3)n2)cc1]

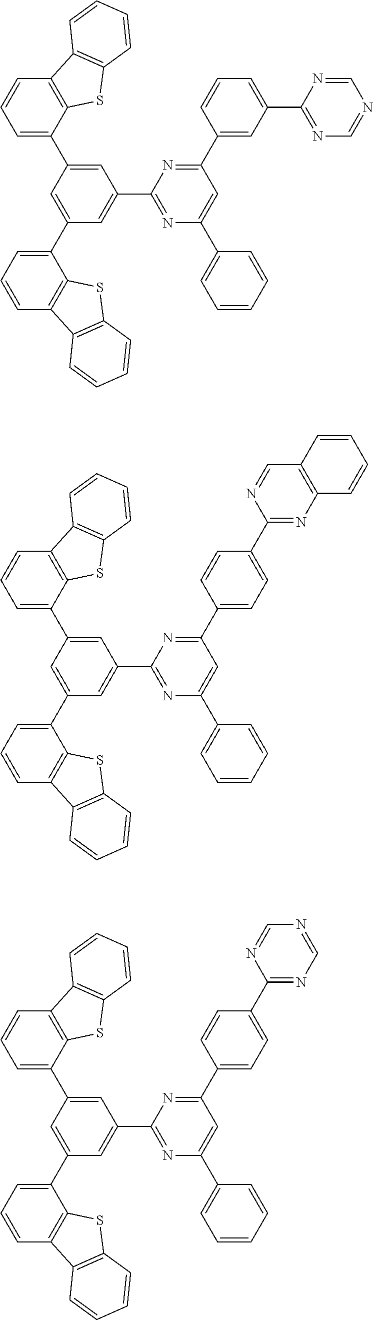 [SMILES: c1ccc(-c2cc(-c3cccc(-c4ncncn4)c3)nc(-c3cc(-c4cccc5c4sc4ccccc45)cc(-c4cccc5c4sc4ccccc45)c3)n2)cc1 ; c1ccc(-c2cc(-c3ccc(-c4ncc5ccccc5n4)cc3)nc(-c3cc(-c4cccc5c4sc4ccccc45)cc(-c4cccc5c4sc4ccccc45)c3)n2)cc1 ; c1ccc(-c2cc(-c3ccc(-c4ncncn4)cc3)nc(-c3cc(-c4cccc5c4sc4ccccc45)cc(-c4cccc5c4sc4ccccc45)c3)n2)cc1]

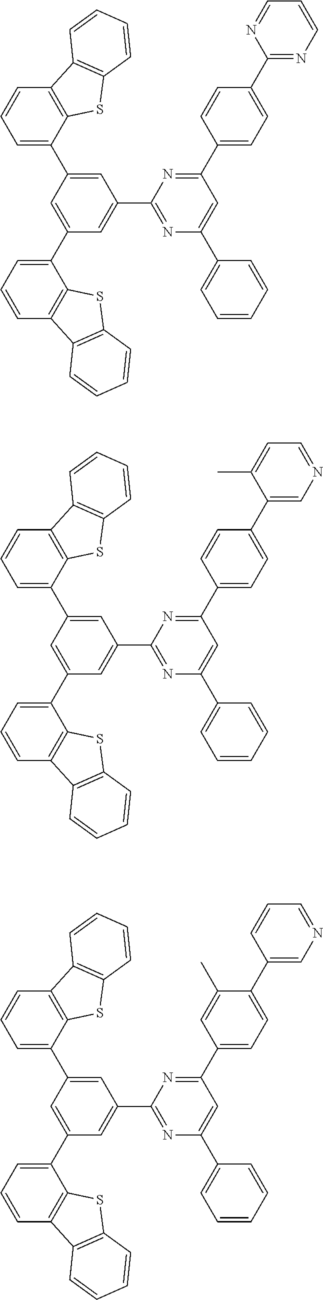 [SMILES: c1ccc(-c2cc(-c3ccc(-c4ncccn4)cc3)nc(-c3cc(-c4cccc5c4sc4ccccc45)cc(-c4cccc5c4sc4ccccc45)c3)n2)cc1 ; Cc1ccncc1-c1ccc(-c2cc(-c3ccccc3)nc(-c3cc(-c4cccc5c4sc4ccccc45)cc(-c4cccc5c4sc4ccccc45)c3)n2)cc1 ; Cc1cc(-c2cc(-c3ccccc3)nc(-c3cc(-c4cccc5c4sc4ccccc45)cc(-c4cccc5c4sc4ccccc45)c3)n2)ccc1-c1cccnc1]

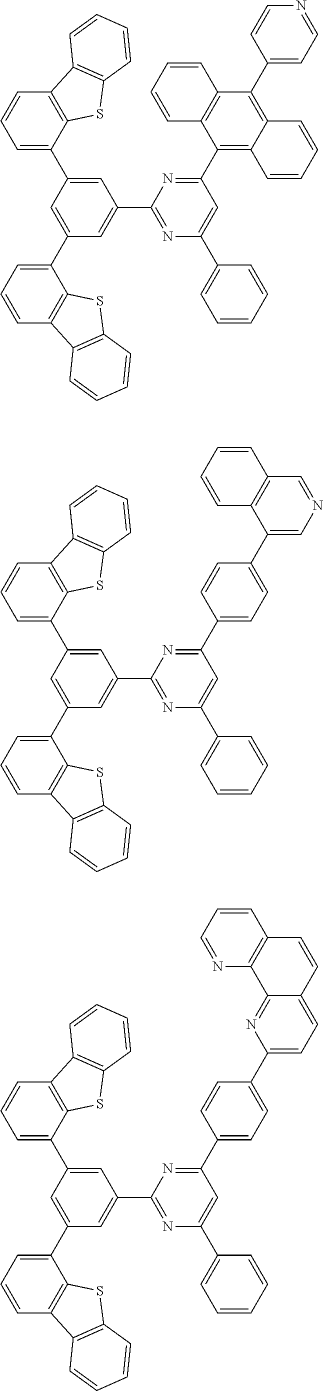 [SMILES: c1ccc(-c2cc(-c3c4ccccc4c(-c4ccncc4)c4ccccc34)nc(-c3cc(-c4cccc5c4sc4ccccc45)cc(-c4cccc5c4sc4ccccc45)c3)n2)cc1 ; c1ccc(-c2cc(-c3ccc(-c4cncc5ccccc45)cc3)nc(-c3cc(-c4cccc5c4sc4ccccc45)cc(-c4cccc5c4sc4ccccc45)c3)n2)cc1 ; c1ccc(-c2cc(-c3ccc(-c4ccc5ccc6cccnc6c5n4)cc3)nc(-c3cc(-c4cccc5c4sc4ccccc45)cc(-c4cccc5c4sc4ccccc45)c3)n2)cc1]

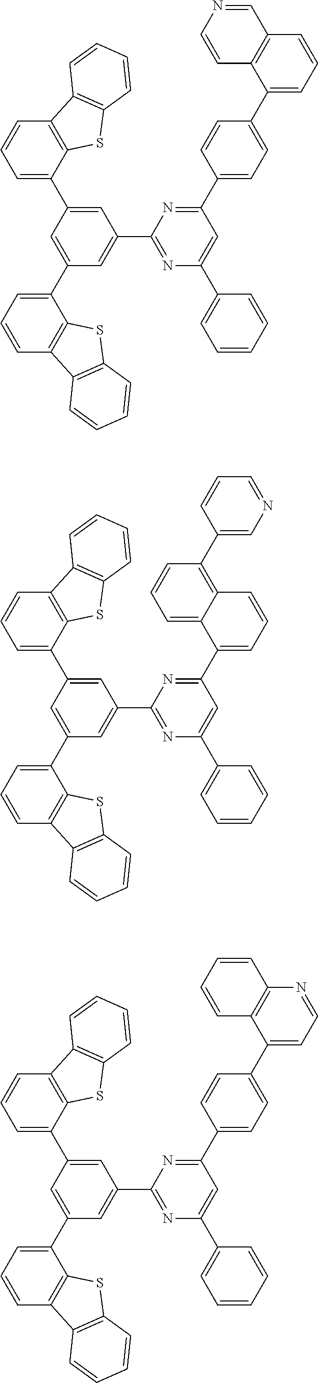 [SMILES: c1ccc(-c2cc(-c3ccc(-c4cccc5cnccc45)cc3)nc(-c3cc(-c4cccc5c4sc4ccccc45)cc(-c4cccc5c4sc4ccccc45)c3)n2)cc1 ; c1ccc(-c2cc(-c3cccc4c(-c5cccnc5)cccc34)nc(-c3cc(-c4cccc5c4sc4ccccc45)cc(-c4cccc5c4sc4ccccc45)c3)n2)cc1 ; c1ccc(-c2cc(-c3ccc(-c4ccnc5ccccc45)cc3)nc(-c3cc(-c4cccc5c4sc4ccccc45)cc(-c4cccc5c4sc4ccccc45)c3)n2)cc1]

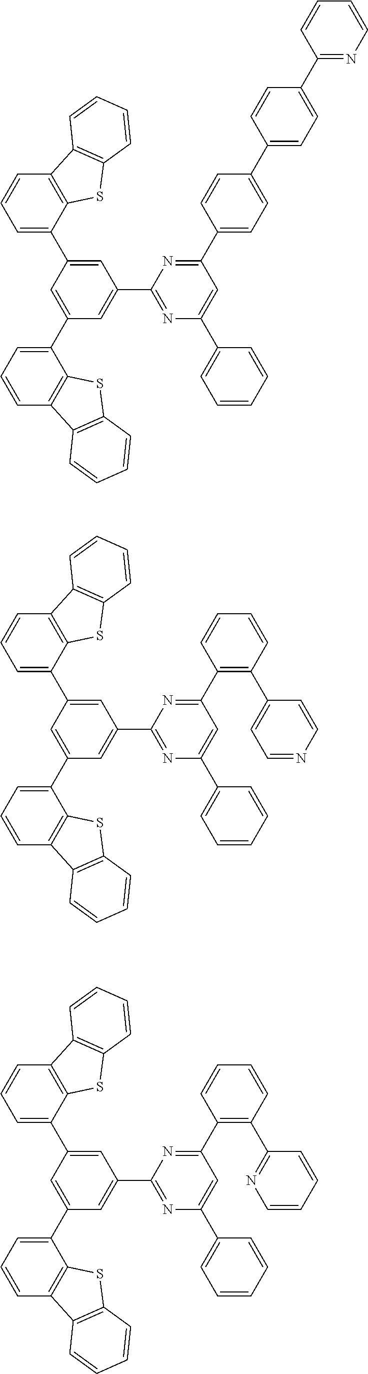 [SMILES: c1ccc(-c2cc(-c3ccc(-c4ccc(-c5ccccn5)cc4)cc3)nc(-c3cc(-c4cccc5c4sc4ccccc45)cc(-c4cccc5c4sc4ccccc45)c3)n2)cc1 ; c1ccc(-c2cc(-c3ccccc3-c3ccncc3)nc(-c3cc(-c4cccc5c4sc4ccccc45)cc(-c4cccc5c4sc4ccccc45)c3)n2)cc1 ; c1ccc(-c2cc(-c3ccccc3-c3ccccn3)nc(-c3cc(-c4cccc5c4sc4ccccc45)cc(-c4cccc5c4sc4ccccc45)c3)n2)cc1]

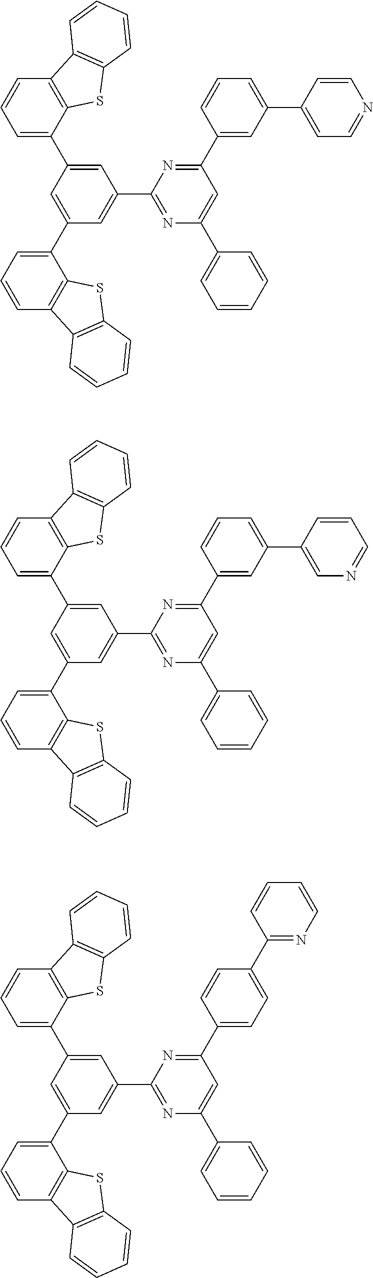 [SMILES: c1ccc(-c2cc(-c3cccc(-c4ccncc4)c3)nc(-c3cc(-c4cccc5c4sc4ccccc45)cc(-c4cccc5c4sc4ccccc45)c3)n2)cc1 ; c1ccc(-c2cc(-c3cccc(-c4cccnc4)c3)nc(-c3cc(-c4cccc5c4sc4ccccc45)cc(-c4cccc5c4sc4ccccc45)c3)n2)cc1 ; c1ccc(-c2cc(-c3ccc(-c4ccccn4)cc3)nc(-c3cc(-c4cccc5c4sc4ccccc45)cc(-c4cccc5c4sc4ccccc45)c3)n2)cc1]

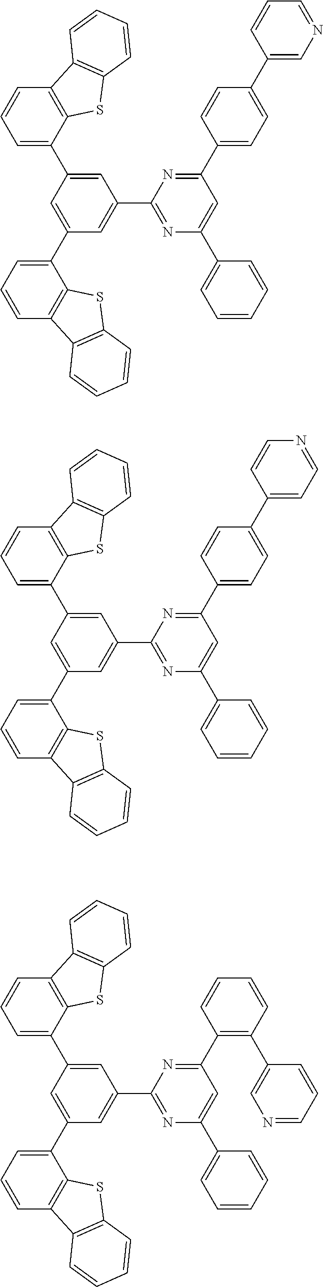 [SMILES: c1ccc(-c2cc(-c3ccc(-c4cccnc4)cc3)nc(-c3cc(-c4cccc5c4sc4ccccc45)cc(-c4cccc5c4sc4ccccc45)c3)n2)cc1 ; c1ccc(-c2cc(-c3ccc(-c4ccncc4)cc3)nc(-c3cc(-c4cccc5c4sc4ccccc45)cc(-c4cccc5c4sc4ccccc45)c3)n2)cc1 ; c1ccc(-c2cc(-c3ccccc3-c3cccnc3)nc(-c3cc(-c4cccc5c4sc4ccccc45)cc(-c4cccc5c4sc4ccccc45)c3)n2)cc1]

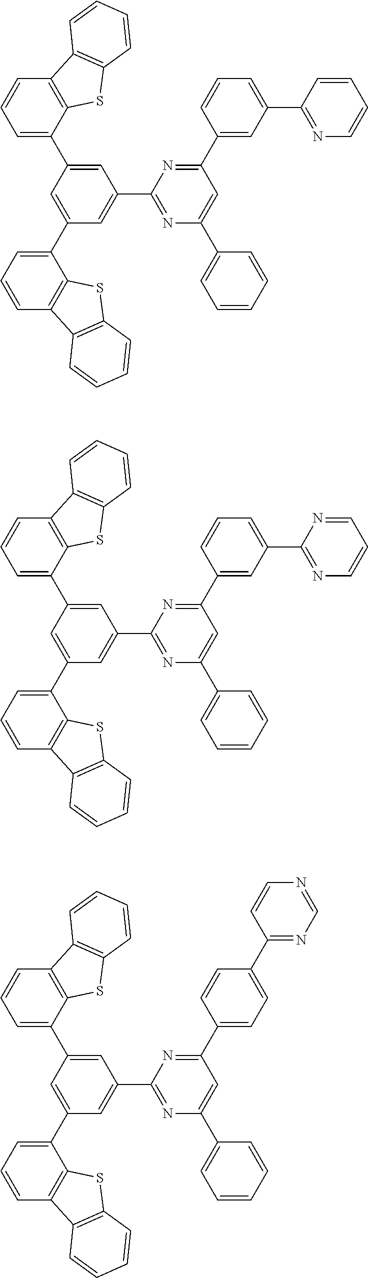 [SMILES: c1ccc(-c2cc(-c3cccc(-c4ccccn4)c3)nc(-c3cc(-c4cccc5c4sc4ccccc45)cc(-c4cccc5c4sc4ccccc45)c3)n2)cc1 ; c1ccc(-c2cc(-c3cccc(-c4ncccn4)c3)nc(-c3cc(-c4cccc5c4sc4ccccc45)cc(-c4cccc5c4sc4ccccc45)c3)n2)cc1 ; c1ccc(-c2cc(-c3ccc(-c4ccncn4)cc3)nc(-c3cc(-c4cccc5c4sc4ccccc45)cc(-c4cccc5c4sc4ccccc45)c3)n2)cc1]

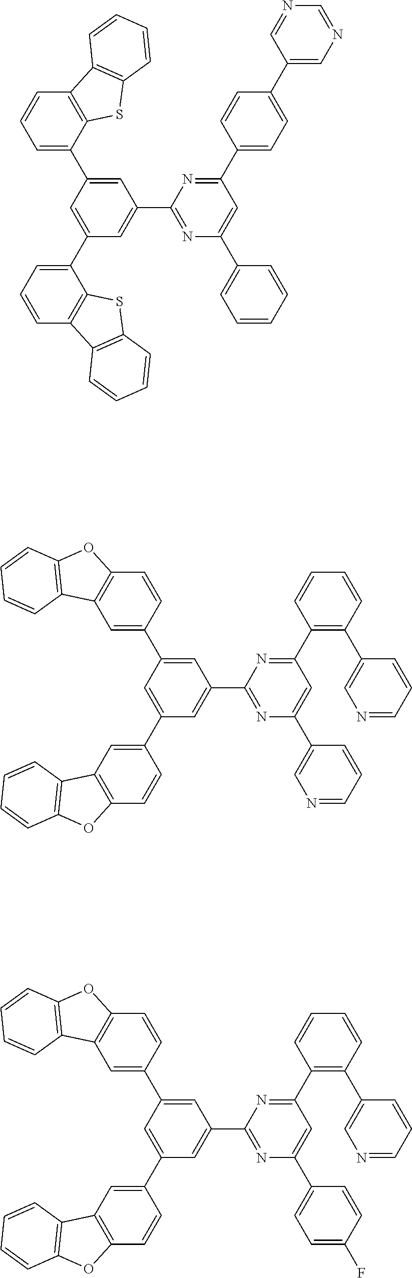 [SMILES: c1ccc(-c2cc(-c3ccc(-c4cncnc4)cc3)nc(-c3cc(-c4cccc5c4sc4ccccc45)cc(-c4cccc5c4sc4ccccc45)c3)n2)cc1 ; c1cncc(-c2cc(-c3ccccc3-c3cccnc3)nc(-c3cc(-c4ccc5oc6ccccc6c5c4)cc(-c4ccc5oc6ccccc6c5c4)c3)n2)c1 ; Fc1ccc(-c2cc(-c3ccccc3-c3cccnc3)nc(-c3cc(-c4ccc5oc6ccccc6c5c4)cc(-c4ccc5oc6ccccc6c5c4)c3)n2)cc1]

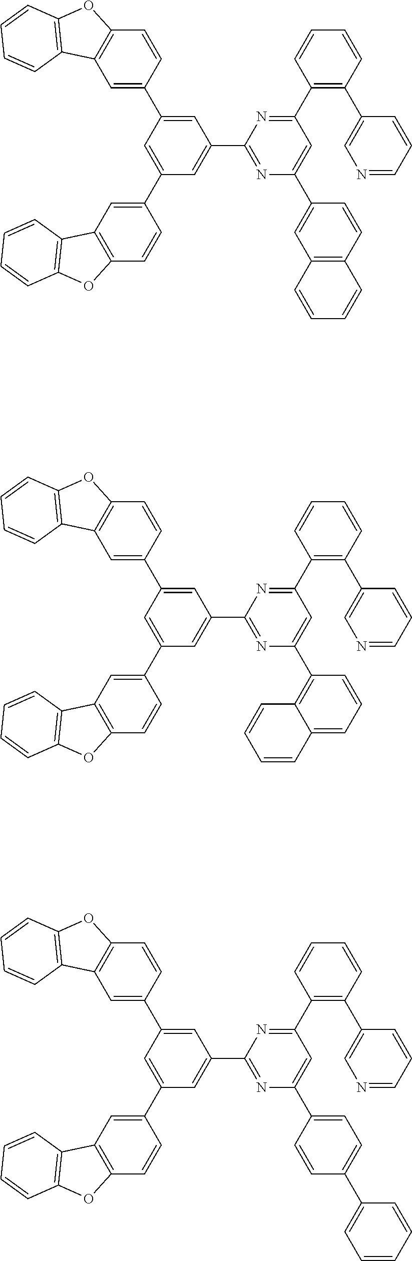 [SMILES: c1cncc(-c2ccccc2-c2cc(-c3ccc4ccccc4c3)nc(-c3cc(-c4ccc5oc6ccccc6c5c4)cc(-c4ccc5oc6ccccc6c5c4)c3)n2)c1 ; c1cncc(-c2ccccc2-c2cc(-c3cccc4ccccc34)nc(-c3cc(-c4ccc5oc6ccccc6c5c4)cc(-c4ccc5oc6ccccc6c5c4)c3)n2)c1 ; c1ccc(-c2ccc(-c3cc(-c4ccccc4-c4cccnc4)nc(-c4cc(-c5ccc6oc7ccccc7c6c5)cc(-c5ccc6oc7ccccc7c6c5)c4)n3)cc2)cc1]

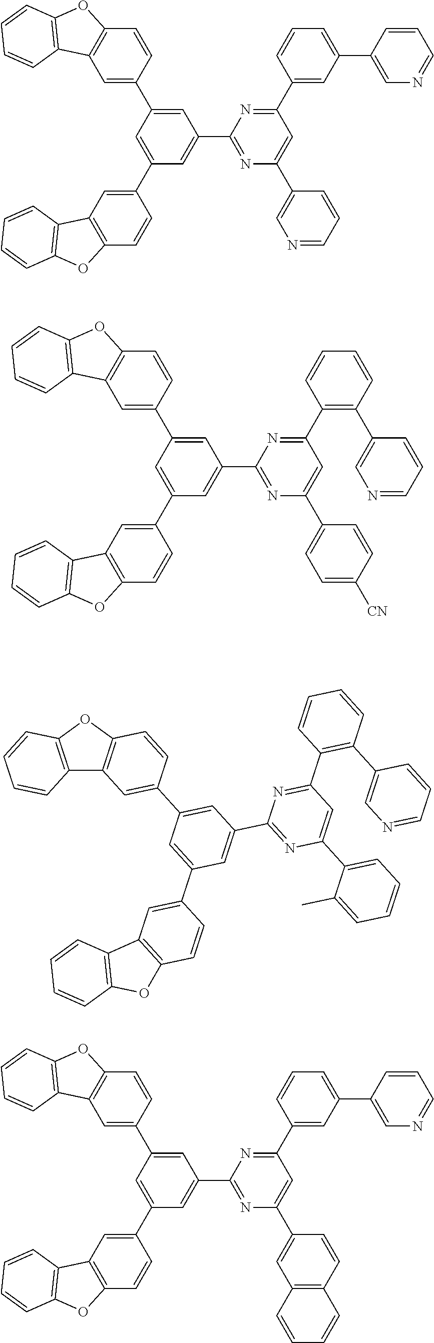

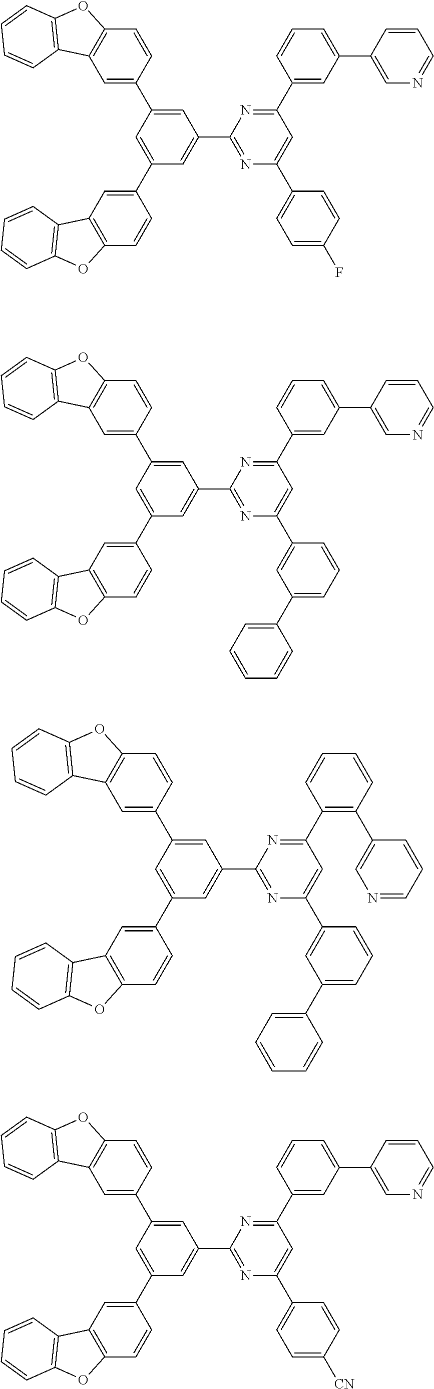 [SMILES: Fc1ccc(-c2cc(-c3cccc(-c4cccnc4)c3)nc(-c3cc(-c4ccc5oc6ccccc6c5c4)cc(-c4ccc5oc6ccccc6c5c4)c3)n2)cc1 ; c1ccc(-c2cccc(-c3cc(-c4cccc(-c5cccnc5)c4)nc(-c4cc(-c5ccc6oc7ccccc7c6c5)cc(-c5ccc6oc7ccccc7c6c5)c4)n3)c2)cc1 ; c1ccc(-c2cccc(-c3cc(-c4ccccc4-c4cccnc4)nc(-c4cc(-c5ccc6oc7ccccc7c6c5)cc(-c5ccc6oc7ccccc7c6c5)c4)n3)c2)cc1 ; N#Cc1ccc(-c2cc(-c3cccc(-c4cccnc4)c3)nc(-c3cc(-c4ccc5oc6ccccc6c5c4)cc(-c4ccc5oc6ccccc6c5c4)c3)n2)cc1]

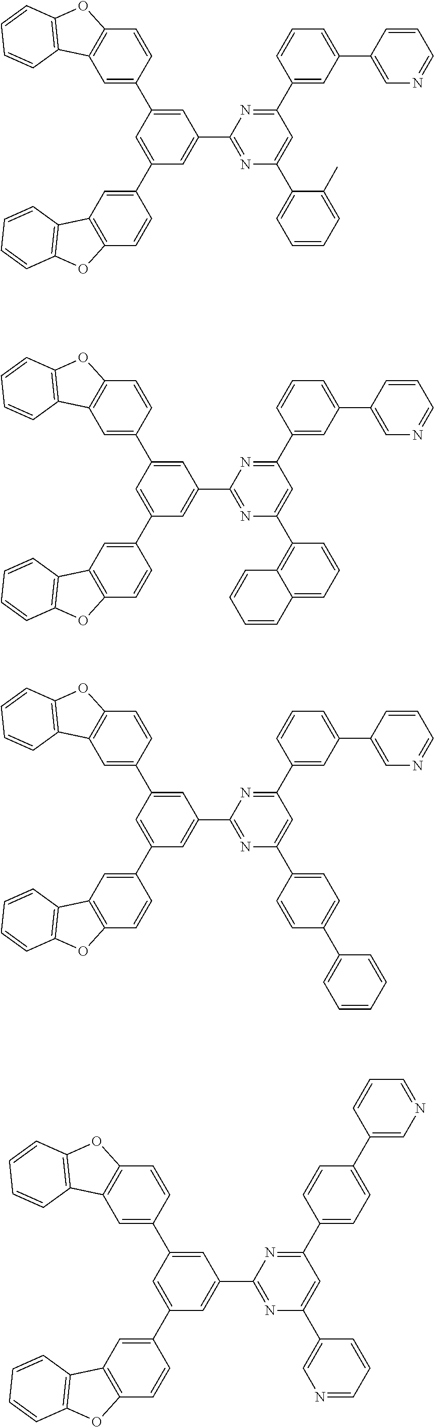 [SMILES: Cc1ccccc1-c1cc(-c2cccc(-c3cccnc3)c2)nc(-c2cc(-c3ccc4oc5ccccc5c4c3)cc(-c3ccc4oc5ccccc5c4c3)c2)n1 ; c1cncc(-c2cccc(-c3cc(-c4cccc5ccccc45)nc(-c4cc(-c5ccc6oc7ccccc7c6c5)cc(-c5ccc6oc7ccccc7c6c5)c4)n3)c2)c1 ; c1ccc(-c2ccc(-c3cc(-c4cccc(-c5cccnc5)c4)nc(-c4cc(-c5ccc6oc7ccccc7c6c5)cc(-c5ccc6oc7ccccc7c6c5)c4)n3)cc2)cc1 ; c1cncc(-c2ccc(-c3cc(-c4cccnc4)nc(-c4cc(-c5ccc6oc7ccccc7c6c5)cc(-c5ccc6oc7ccccc7c6c5)c4)n3)cc2)c1]

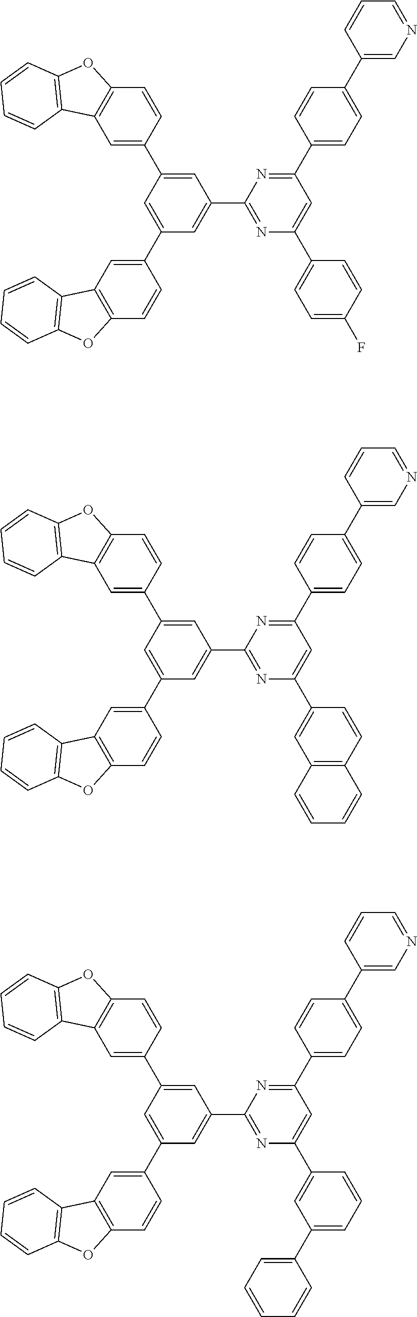 [SMILES: Fc1ccc(-c2cc(-c3ccc(-c4cccnc4)cc3)nc(-c3cc(-c4ccc5oc6ccccc6c5c4)cc(-c4ccc5oc6ccccc6c5c4)c3)n2)cc1 ; c1cncc(-c2ccc(-c3cc(-c4ccc5ccccc5c4)nc(-c4cc(-c5ccc6oc7ccccc7c6c5)cc(-c5ccc6oc7ccccc7c6c5)c4)n3)cc2)c1 ; c1ccc(-c2cccc(-c3cc(-c4ccc(-c5cccnc5)cc4)nc(-c4cc(-c5ccc6oc7ccccc7c6c5)cc(-c5ccc6oc7ccccc7c6c5)c4)n3)c2)cc1]

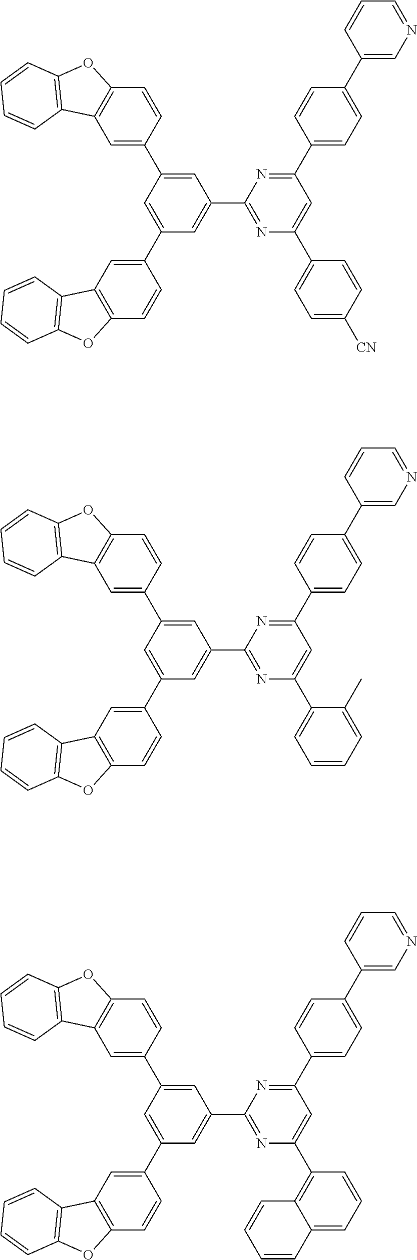 [SMILES: N#Cc1ccc(-c2cc(-c3ccc(-c4cccnc4)cc3)nc(-c3cc(-c4ccc5oc6ccccc6c5c4)cc(-c4ccc5oc6ccccc6c5c4)c3)n2)cc1 ; Cc1ccccc1-c1cc(-c2ccc(-c3cccnc3)cc2)nc(-c2cc(-c3ccc4oc5ccccc5c4c3)cc(-c3ccc4oc5ccccc5c4c3)c2)n1 ; c1cncc(-c2ccc(-c3cc(-c4cccc5ccccc45)nc(-c4cc(-c5ccc6oc7ccccc7c6c5)cc(-c5ccc6oc7ccccc7c6c5)c4)n3)cc2)c1]

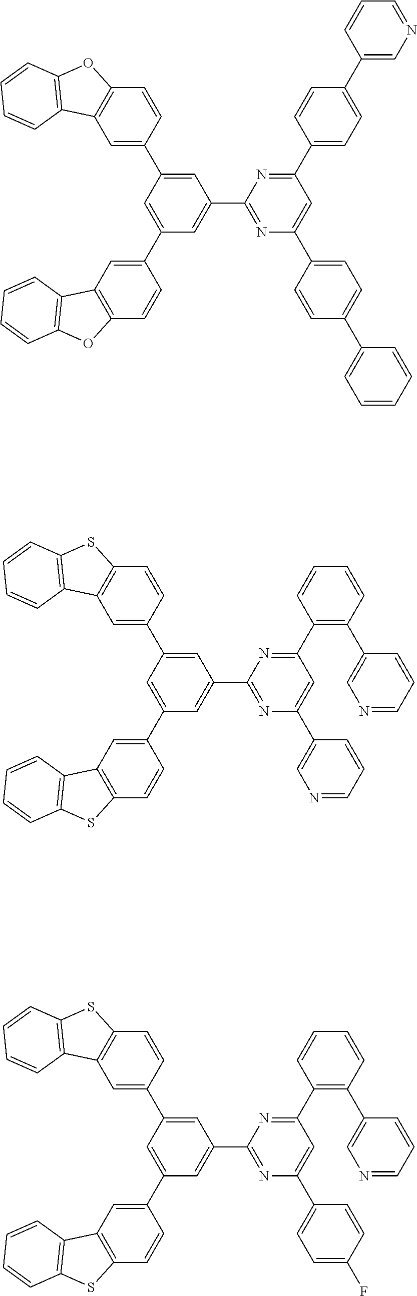 [SMILES: c1ccc(-c2ccc(-c3cc(-c4ccc(-c5cccnc5)cc4)nc(-c4cc(-c5ccc6oc7ccccc7c6c5)cc(-c5ccc6oc7ccccc7c6c5)c4)n3)cc2)cc1 ; c1cncc(-c2cc(-c3ccccc3-c3cccnc3)nc(-c3cc(-c4ccc5sc6ccccc6c5c4)cc(-c4ccc5sc6ccccc6c5c4)c3)n2)c1 ; Fc1ccc(-c2cc(-c3ccccc3-c3cccnc3)nc(-c3cc(-c4ccc5sc6ccccc6c5c4)cc(-c4ccc5sc6ccccc6c5c4)c3)n2)cc1]

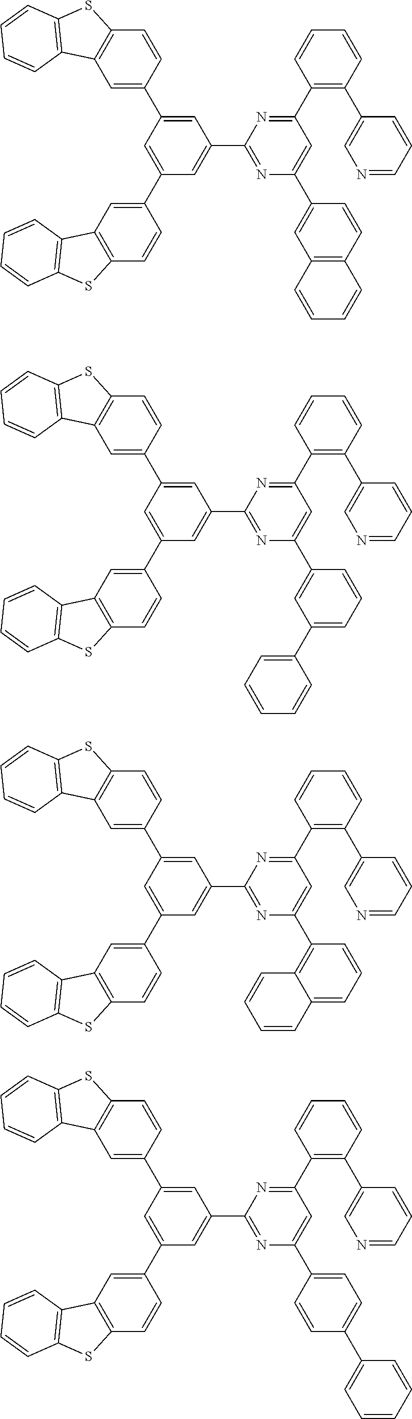 [SMILES: c1cncc(-c2ccccc2-c2cc(-c3ccc4ccccc4c3)nc(-c3cc(-c4ccc5sc6ccccc6c5c4)cc(-c4ccc5sc6ccccc6c5c4)c3)n2)c1 ; c1ccc(-c2cccc(-c3cc(-c4ccccc4-c4cccnc4)nc(-c4cc(-c5ccc6sc7ccccc7c6c5)cc(-c5ccc6sc7ccccc7c6c5)c4)n3)c2)cc1 ; c1cncc(-c2ccccc2-c2cc(-c3cccc4ccccc34)nc(-c3cc(-c4ccc5sc6ccccc6c5c4)cc(-c4ccc5sc6ccccc6c5c4)c3)n2)c1 ; c1ccc(-c2ccc(-c3cc(-c4ccccc4-c4cccnc4)nc(-c4cc(-c5ccc6sc7ccccc7c6c5)cc(-c5ccc6sc7ccccc7c6c5)c4)n3)cc2)cc1]

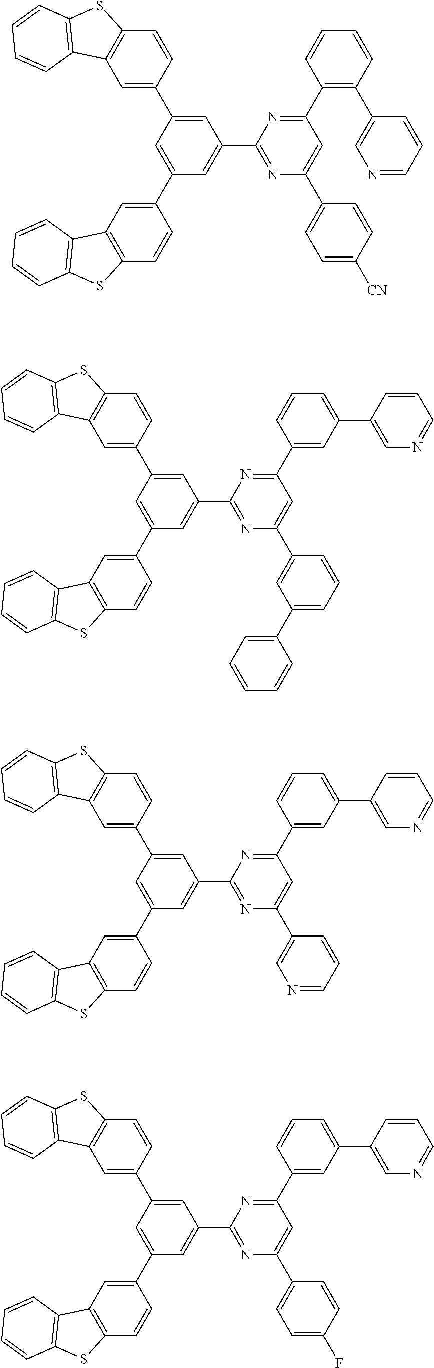 [SMILES: N#Cc1ccc(-c2cc(-c3ccccc3-c3cccnc3)nc(-c3cc(-c4ccc5sc6ccccc6c5c4)cc(-c4ccc5sc6ccccc6c5c4)c3)n2)cc1 ; c1ccc(-c2cccc(-c3cc(-c4cccc(-c5cccnc5)c4)nc(-c4cc(-c5ccc6sc7ccccc7c6c5)cc(-c5ccc6sc7ccccc7c6c5)c4)n3)c2)cc1 ; c1cncc(-c2cccc(-c3cc(-c4cccnc4)nc(-c4cc(-c5ccc6sc7ccccc7c6c5)cc(-c5ccc6sc7ccccc7c6c5)c4)n3)c2)c1 ; Fc1ccc(-c2cc(-c3cccc(-c4cccnc4)c3)nc(-c3cc(-c4ccc5sc6ccccc6c5c4)cc(-c4ccc5sc6ccccc6c5c4)c3)n2)cc1]

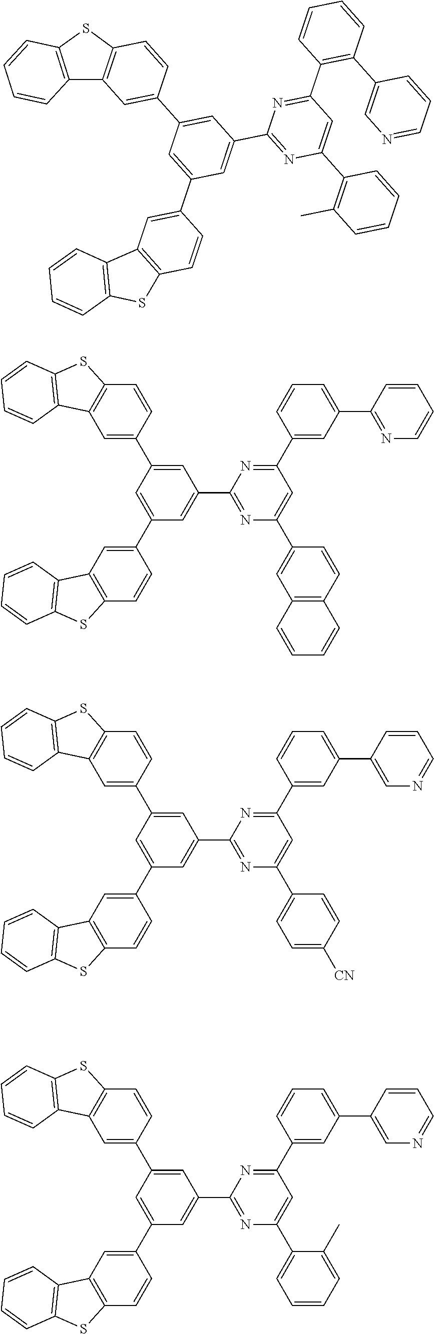 [SMILES: Cc1ccccc1-c1cc(-c2ccccc2-c2cccnc2)nc(-c2cc(-c3ccc4sc5ccccc5c4c3)cc(-c3ccc4sc5ccccc5c4c3)c2)n1 ; c1ccc(-c2cccc(-c3cc(-c4ccc5ccccc5c4)nc(-c4cc(-c5ccc6sc7ccccc7c6c5)cc(-c5ccc6sc7ccccc7c6c5)c4)n3)c2)nc1 ; N#Cc1ccc(-c2cc(-c3cccc(-c4cccnc4)c3)nc(-c3cc(-c4ccc5sc6ccccc6c5c4)cc(-c4ccc5sc6ccccc6c5c4)c3)n2)cc1 ; Cc1ccccc1-c1cc(-c2cccc(-c3cccnc3)c2)nc(-c2cc(-c3ccc4sc5ccccc5c4c3)cc(-c3ccc4sc5ccccc5c4c3)c2)n1]

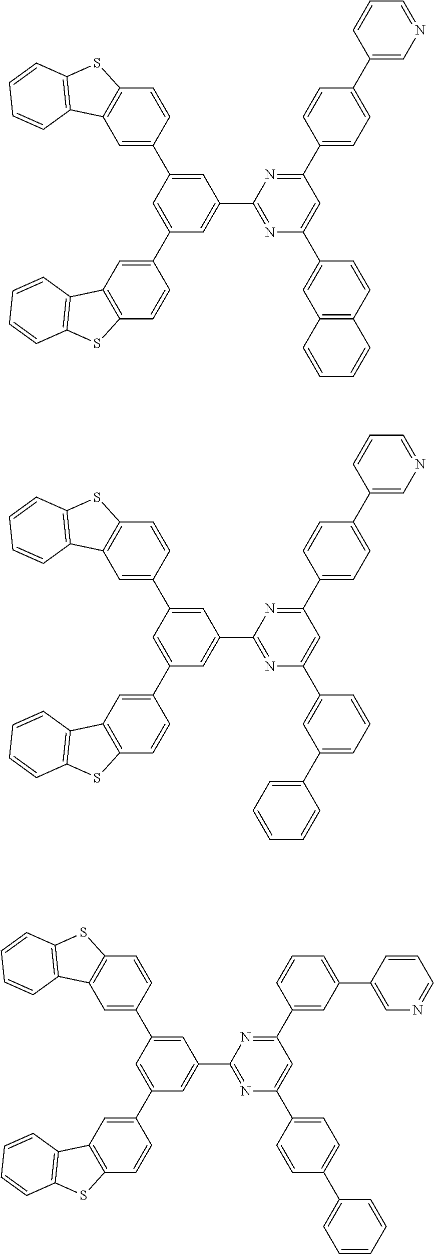 [SMILES: c1cncc(-c2ccc(-c3cc(-c4ccc5ccccc5c4)nc(-c4cc(-c5ccc6sc7ccccc7c6c5)cc(-c5ccc6sc7ccccc7c6c5)c4)n3)cc2)c1 ; c1ccc(-c2cccc(-c3cc(-c4ccc(-c5cccnc5)cc4)nc(-c4cc(-c5ccc6sc7ccccc7c6c5)cc(-c5ccc6sc7ccccc7c6c5)c4)n3)c2)cc1 ; c1ccc(-c2ccc(-c3cc(-c4cccc(-c5cccnc5)c4)nc(-c4cc(-c5ccc6sc7ccccc7c6c5)cc(-c5ccc6sc7ccccc7c6c5)c4)n3)cc2)cc1]

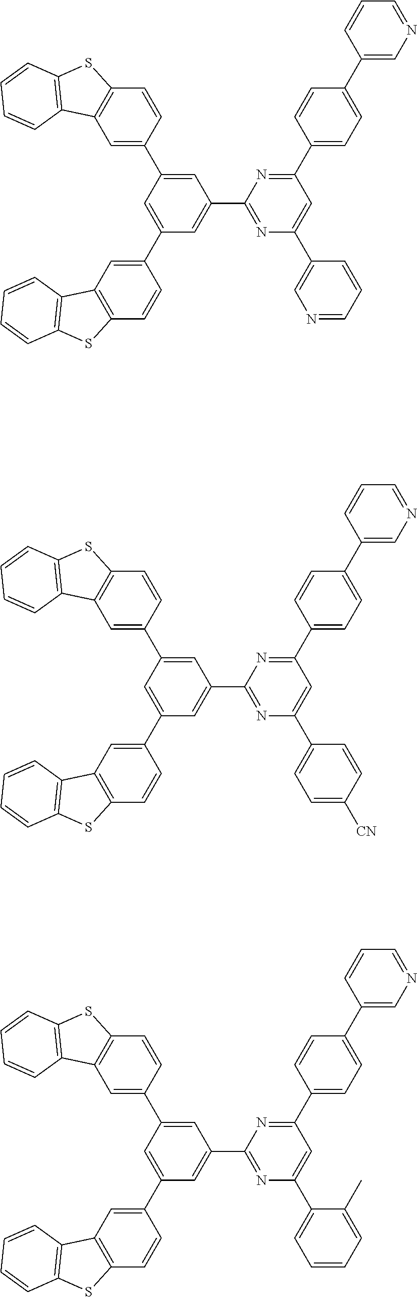 [SMILES: c1cncc(-c2ccc(-c3cc(-c4cccnc4)nc(-c4cc(-c5ccc6sc7ccccc7c6c5)cc(-c5ccc6sc7ccccc7c6c5)c4)n3)cc2)c1 ; N#Cc1ccc(-c2cc(-c3ccc(-c4cccnc4)cc3)nc(-c3cc(-c4ccc5sc6ccccc6c5c4)cc(-c4ccc5sc6ccccc6c5c4)c3)n2)cc1 ; Cc1ccccc1-c1cc(-c2ccc(-c3cccnc3)cc2)nc(-c2cc(-c3ccc4sc5ccccc5c4c3)cc(-c3ccc4sc5ccccc5c4c3)c2)n1]

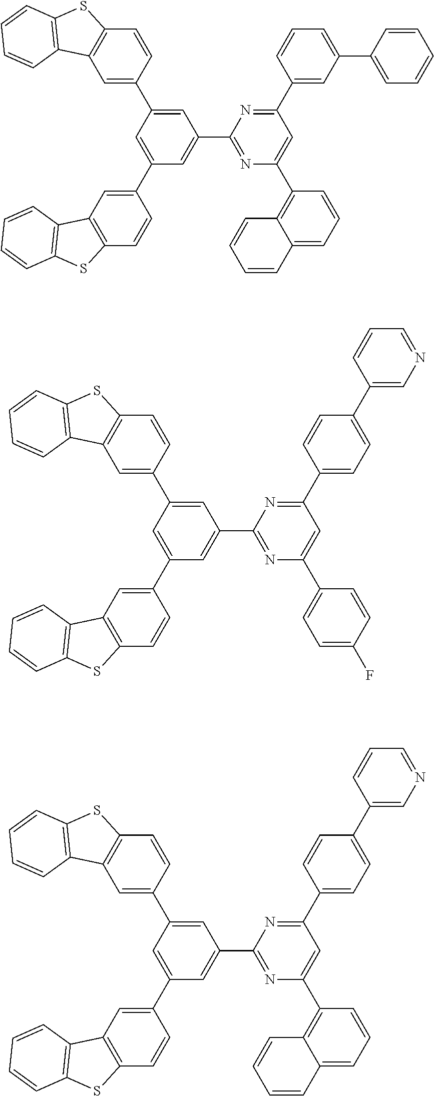 [SMILES: c1ccc(-c2cccc(-c3cc(-c4cccc5ccccc45)nc(-c4cc(-c5ccc6sc7ccccc7c6c5)cc(-c5ccc6sc7ccccc7c6c5)c4)n3)c2)cc1 ; Fc1ccc(-c2cc(-c3ccc(-c4cccnc4)cc3)nc(-c3cc(-c4ccc5sc6ccccc6c5c4)cc(-c4ccc5sc6ccccc6c5c4)c3)n2)cc1 ; c1cncc(-c2ccc(-c3cc(-c4cccc5ccccc45)nc(-c4cc(-c5ccc6sc7ccccc7c6c5)cc(-c5ccc6sc7ccccc7c6c5)c4)n3)cc2)c1]

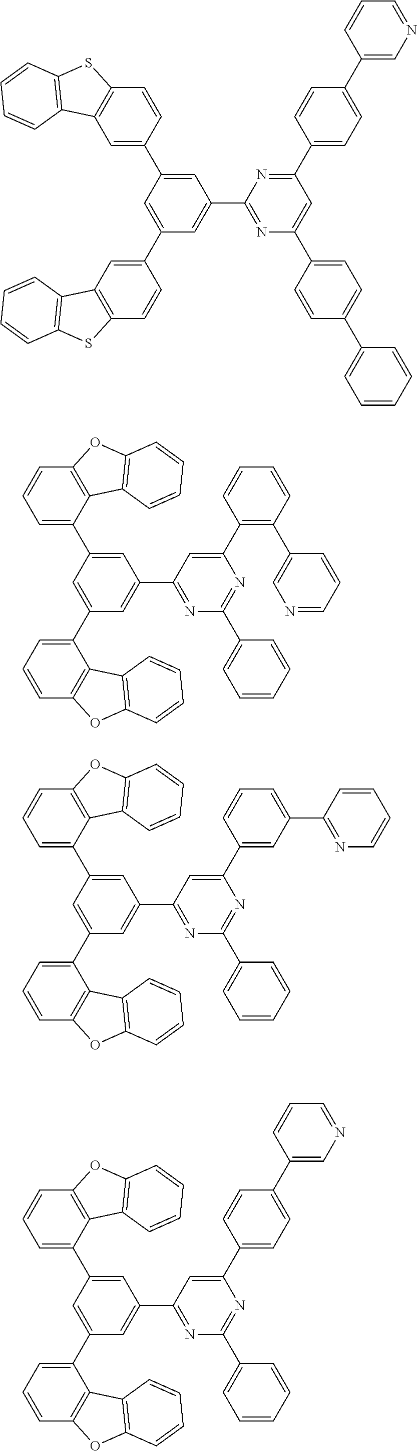

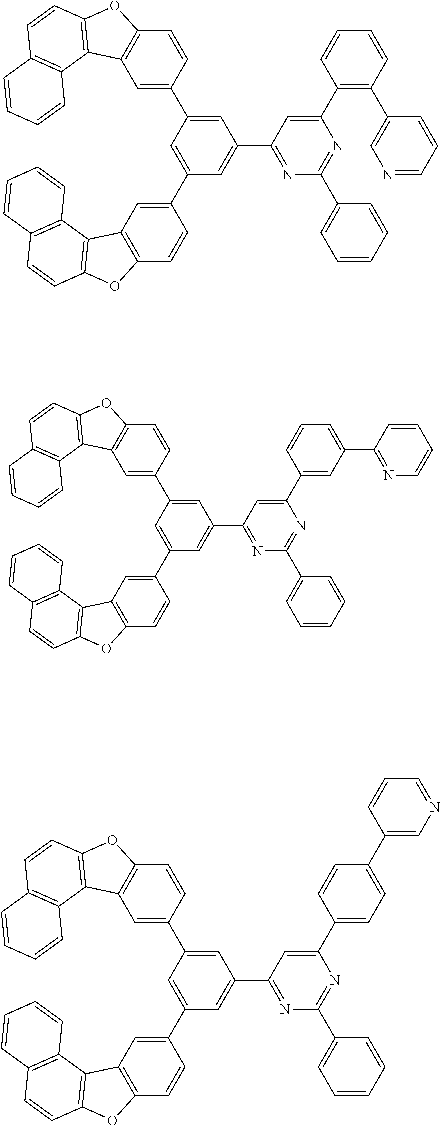 [SMILES: c1ccc(-c2nc(-c3cc(-c4ccc5oc6ccc7ccccc7c6c5c4)cc(-c4ccc5oc6ccc7ccccc7c6c5c4)c3)cc(-c3ccccc3-c3cccnc3)n2)cc1 ; c1ccc(-c2nc(-c3cc(-c4ccc5oc6ccc7ccccc7c6c5c4)cc(-c4ccc5oc6ccc7ccccc7c6c5c4)c3)cc(-c3cccc(-c4ccccn4)c3)n2)cc1 ; c1ccc(-c2nc(-c3ccc(-c4cccnc4)cc3)cc(-c3cc(-c4ccc5oc6ccc7ccccc7c6c5c4)cc(-c4ccc5oc6ccc7ccccc7c6c5c4)c3)n2)cc1]

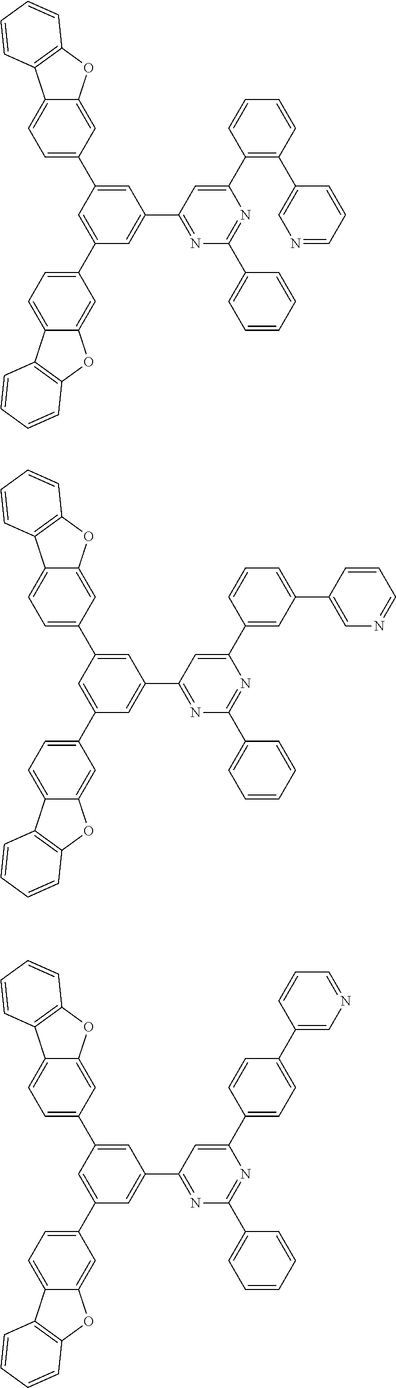

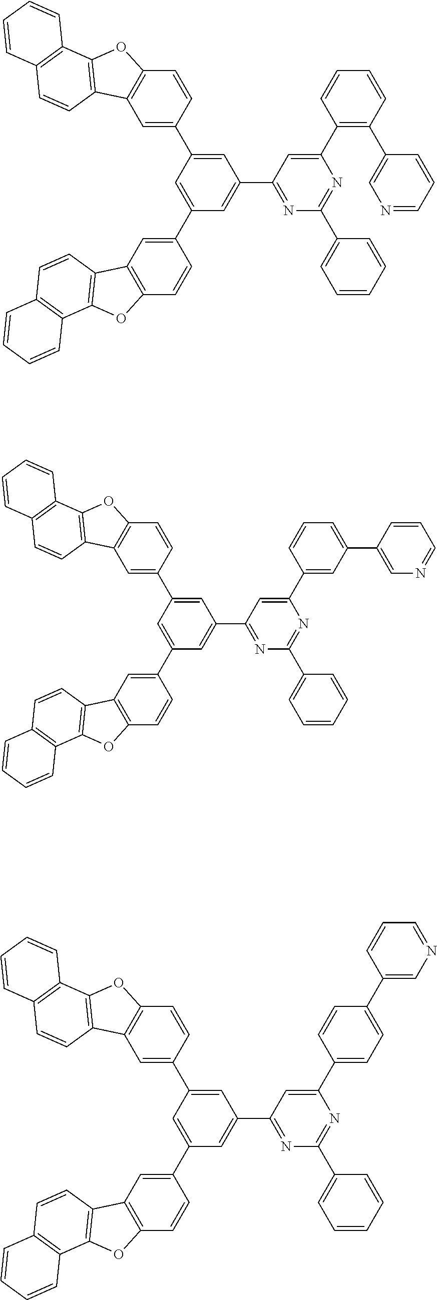 [SMILES: c1ccc(-c2nc(-c3cc(-c4ccc5oc6c7ccccc7ccc6c5c4)cc(-c4ccc5oc6c7ccccc7ccc6c5c4)c3)cc(-c3ccccc3-c3cccnc3)n2)cc1 ; c1ccc(-c2nc(-c3cccc(-c4cccnc4)c3)cc(-c3cc(-c4ccc5oc6c7ccccc7ccc6c5c4)cc(-c4ccc5oc6c7ccccc7ccc6c5c4)c3)n2)cc1 ; c1ccc(-c2nc(-c3ccc(-c4cccnc4)cc3)cc(-c3cc(-c4ccc5oc6c7ccccc7ccc6c5c4)cc(-c4ccc5oc6c7ccccc7ccc6c5c4)c3)n2)cc1]

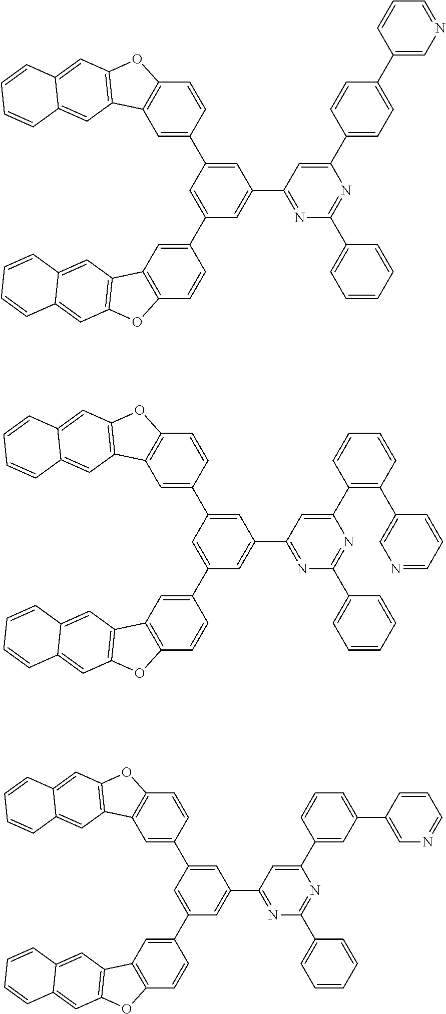 [SMILES: c1ccc(-c2nc(-c3ccc(-c4cccnc4)cc3)cc(-c3cc(-c4ccc5oc6cc7ccccc7cc6c5c4)cc(-c4ccc5oc6cc7ccccc7cc6c5c4)c3)n2)cc1 ; c1ccc(-c2nc(-c3cc(-c4ccc5oc6cc7ccccc7cc6c5c4)cc(-c4ccc5oc6cc7ccccc7cc6c5c4)c3)cc(-c3ccccc3-c3cccnc3)n2)cc1 ; c1ccc(-c2nc(-c3cccc(-c4cccnc4)c3)cc(-c3cc(-c4ccc5oc6cc7ccccc7cc6c5c4)cc(-c4ccc5oc6cc7ccccc7cc6c5c4)c3)n2)cc1]

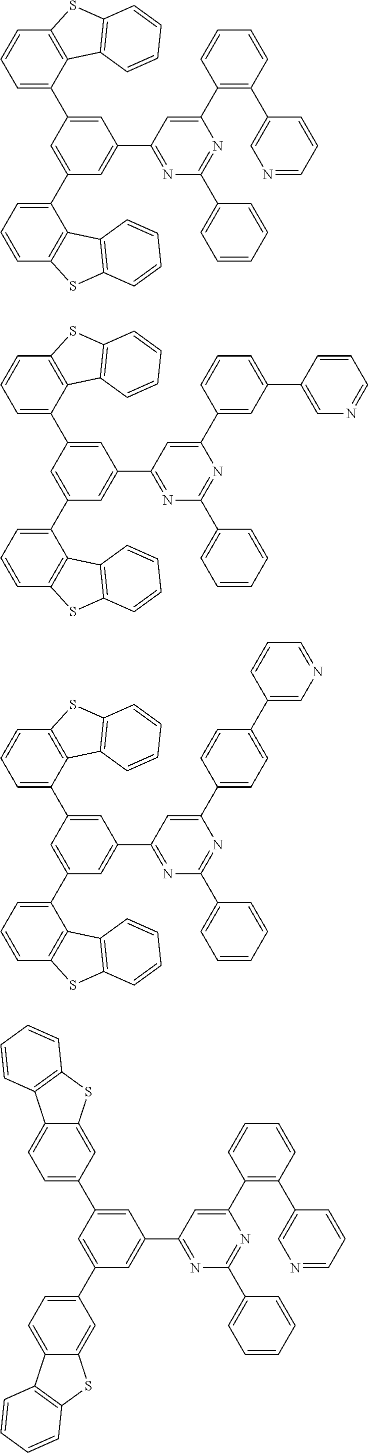

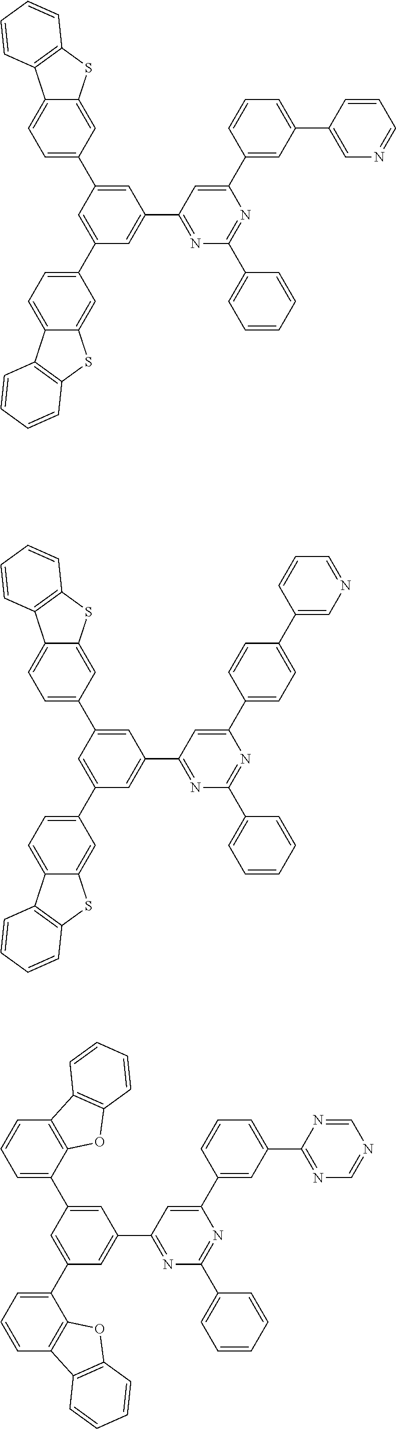 [SMILES: c1ccc(-c2nc(-c3cccc(-c4cccnc4)c3)cc(-c3cc(-c4ccc5c(c4)sc4ccccc45)cc(-c4ccc5c(c4)sc4ccccc45)c3)n2)cc1 ; c1ccc(-c2nc(-c3ccc(-c4cccnc4)cc3)cc(-c3cc(-c4ccc5c(c4)sc4ccccc45)cc(-c4ccc5c(c4)sc4ccccc45)c3)n2)cc1 ; c1ccc(-c2nc(-c3cccc(-c4ncncn4)c3)cc(-c3cc(-c4cccc5c4oc4ccccc45)cc(-c4cccc5c4oc4ccccc45)c3)n2)cc1]

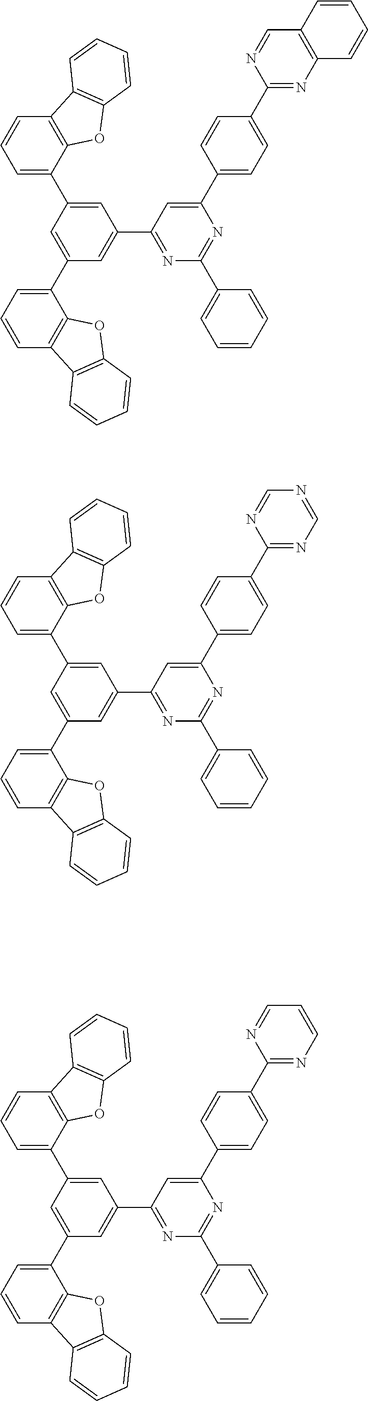 [SMILES: c1ccc(-c2nc(-c3ccc(-c4ncc5ccccc5n4)cc3)cc(-c3cc(-c4cccc5c4oc4ccccc45)cc(-c4cccc5c4oc4ccccc45)c3)n2)cc1 ; c1ccc(-c2nc(-c3ccc(-c4ncncn4)cc3)cc(-c3cc(-c4cccc5c4oc4ccccc45)cc(-c4cccc5c4oc4ccccc45)c3)n2)cc1 ; c1ccc(-c2nc(-c3ccc(-c4ncccn4)cc3)cc(-c3cc(-c4cccc5c4oc4ccccc45)cc(-c4cccc5c4oc4ccccc45)c3)n2)cc1]

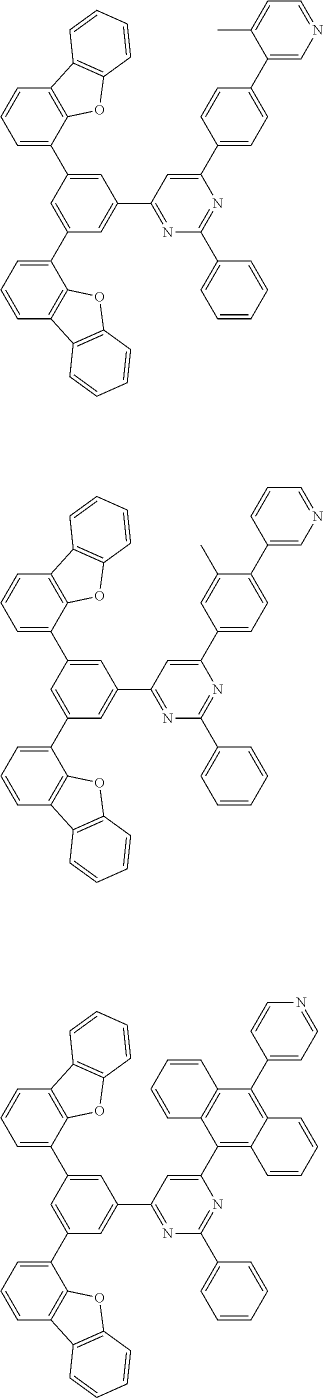 [SMILES: Cc1ccncc1-c1ccc(-c2cc(-c3cc(-c4cccc5c4oc4ccccc45)cc(-c4cccc5c4oc4ccccc45)c3)nc(-c3ccccc3)n2)cc1 ; Cc1cc(-c2cc(-c3cc(-c4cccc5c4oc4ccccc45)cc(-c4cccc5c4oc4ccccc45)c3)nc(-c3ccccc3)n2)ccc1-c1cccnc1 ; c1ccc(-c2nc(-c3cc(-c4cccc5c4oc4ccccc45)cc(-c4cccc5c4oc4ccccc45)c3)cc(-c3c4ccccc4c(-c4ccncc4)c4ccccc34)n2)cc1]

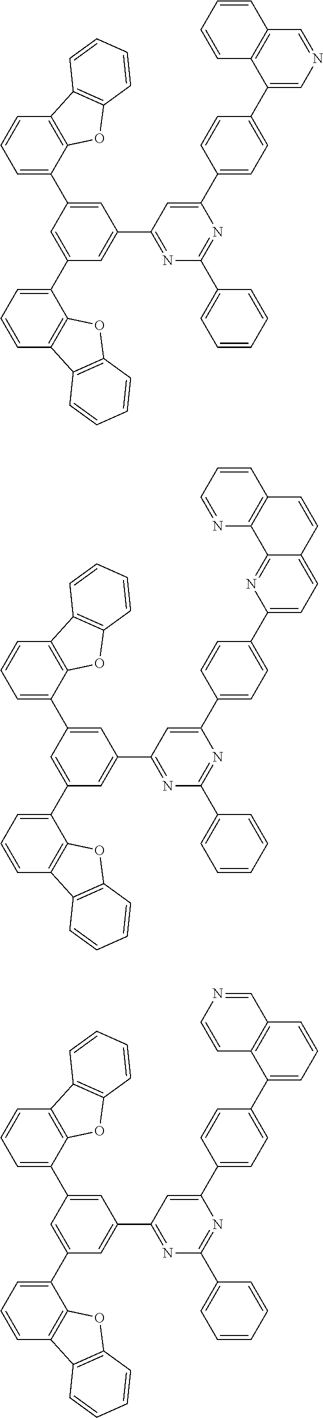 [SMILES: c1ccc(-c2nc(-c3ccc(-c4cncc5ccccc45)cc3)cc(-c3cc(-c4cccc5c4oc4ccccc45)cc(-c4cccc5c4oc4ccccc45)c3)n2)cc1 ; c1ccc(-c2nc(-c3ccc(-c4ccc5ccc6cccnc6c5n4)cc3)cc(-c3cc(-c4cccc5c4oc4ccccc45)cc(-c4cccc5c4oc4ccccc45)c3)n2)cc1 ; c1ccc(-c2nc(-c3ccc(-c4cccc5cnccc45)cc3)cc(-c3cc(-c4cccc5c4oc4ccccc45)cc(-c4cccc5c4oc4ccccc45)c3)n2)cc1]

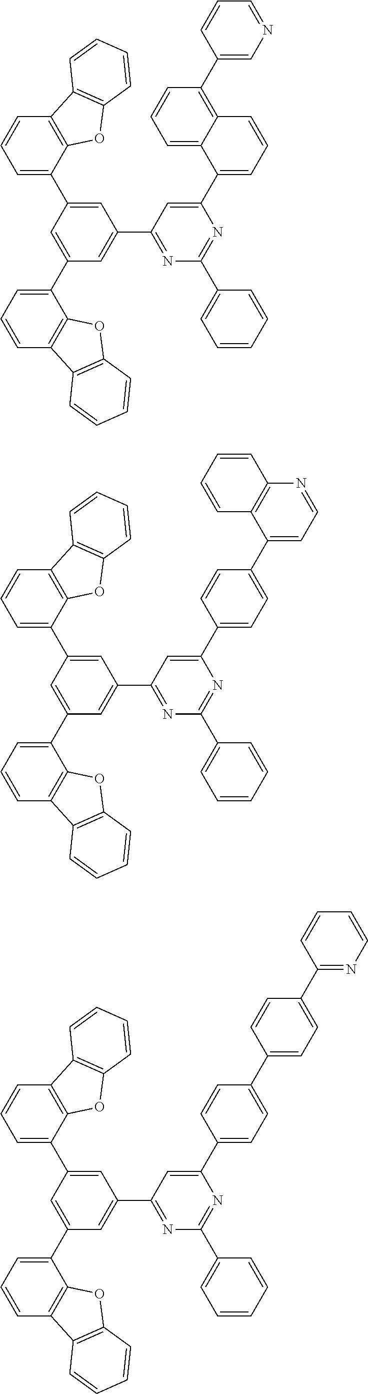 [SMILES: c1ccc(-c2nc(-c3cc(-c4cccc5c4oc4ccccc45)cc(-c4cccc5c4oc4ccccc45)c3)cc(-c3cccc4c(-c5cccnc5)cccc34)n2)cc1 ; c1ccc(-c2nc(-c3ccc(-c4ccnc5ccccc45)cc3)cc(-c3cc(-c4cccc5c4oc4ccccc45)cc(-c4cccc5c4oc4ccccc45)c3)n2)cc1 ; c1ccc(-c2nc(-c3ccc(-c4ccc(-c5ccccn5)cc4)cc3)cc(-c3cc(-c4cccc5c4oc4ccccc45)cc(-c4cccc5c4oc4ccccc45)c3)n2)cc1]

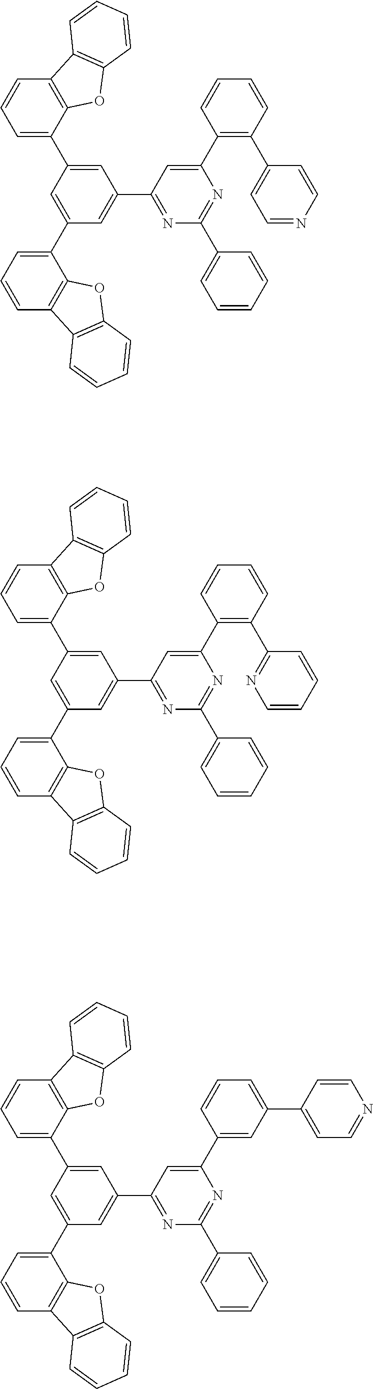 [SMILES: c1ccc(-c2nc(-c3cc(-c4cccc5c4oc4ccccc45)cc(-c4cccc5c4oc4ccccc45)c3)cc(-c3ccccc3-c3ccncc3)n2)cc1 ; c1ccc(-c2nc(-c3cc(-c4cccc5c4oc4ccccc45)cc(-c4cccc5c4oc4ccccc45)c3)cc(-c3ccccc3-c3ccccn3)n2)cc1 ; c1ccc(-c2nc(-c3cccc(-c4ccncc4)c3)cc(-c3cc(-c4cccc5c4oc4ccccc45)cc(-c4cccc5c4oc4ccccc45)c3)n2)cc1]

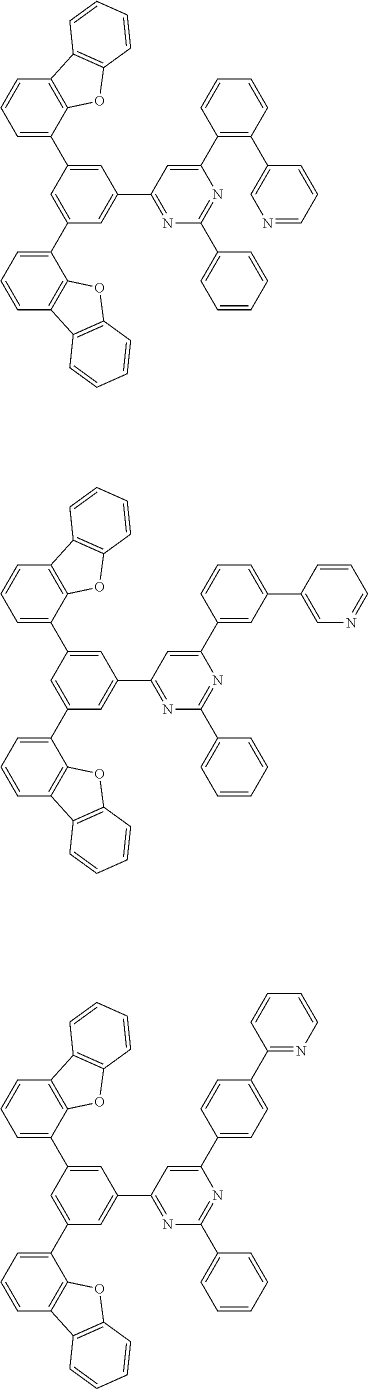 [SMILES: c1ccc(-c2nc(-c3cc(-c4cccc5c4oc4ccccc45)cc(-c4cccc5c4oc4ccccc45)c3)cc(-c3ccccc3-c3cccnc3)n2)cc1 ; c1ccc(-c2nc(-c3cccc(-c4cccnc4)c3)cc(-c3cc(-c4cccc5c4oc4ccccc45)cc(-c4cccc5c4oc4ccccc45)c3)n2)cc1 ; c1ccc(-c2nc(-c3ccc(-c4ccccn4)cc3)cc(-c3cc(-c4cccc5c4oc4ccccc45)cc(-c4cccc5c4oc4ccccc45)c3)n2)cc1]

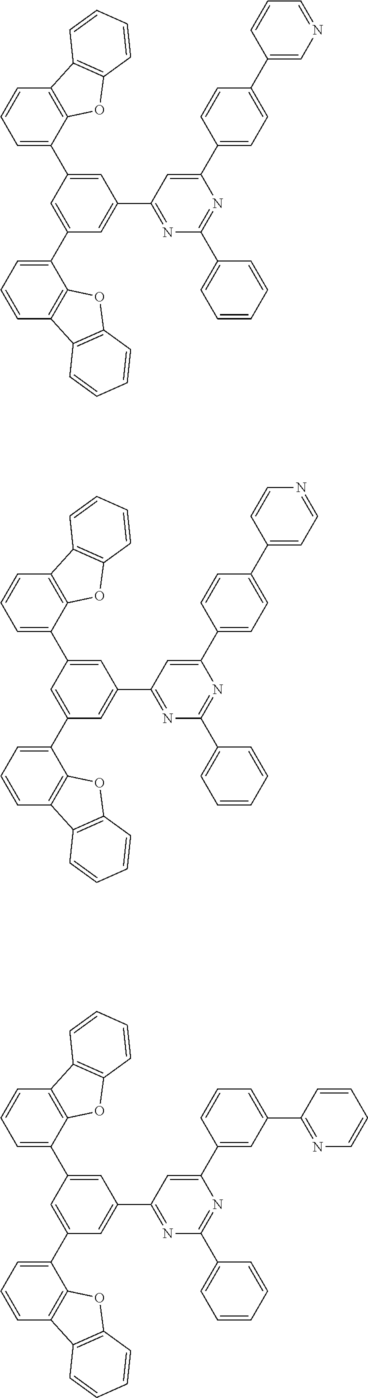 [SMILES: c1ccc(-c2nc(-c3ccc(-c4cccnc4)cc3)cc(-c3cc(-c4cccc5c4oc4ccccc45)cc(-c4cccc5c4oc4ccccc45)c3)n2)cc1 ; c1ccc(-c2nc(-c3ccc(-c4ccncc4)cc3)cc(-c3cc(-c4cccc5c4oc4ccccc45)cc(-c4cccc5c4oc4ccccc45)c3)n2)cc1 ; c1ccc(-c2nc(-c3cccc(-c4ccccn4)c3)cc(-c3cc(-c4cccc5c4oc4ccccc45)cc(-c4cccc5c4oc4ccccc45)c3)n2)cc1]

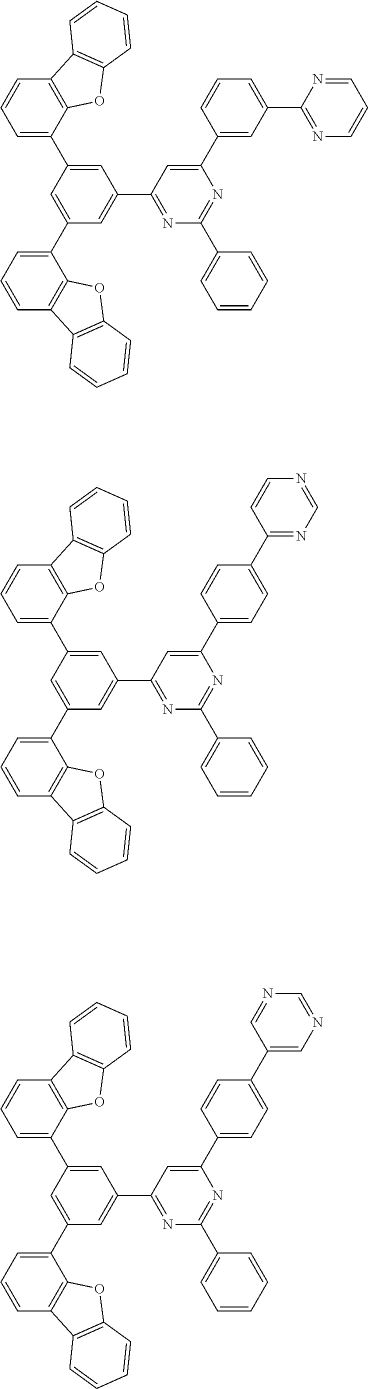 [SMILES: c1ccc(-c2nc(-c3cccc(-c4ncccn4)c3)cc(-c3cc(-c4cccc5c4oc4ccccc45)cc(-c4cccc5c4oc4ccccc45)c3)n2)cc1 ; c1ccc(-c2nc(-c3ccc(-c4ccncn4)cc3)cc(-c3cc(-c4cccc5c4oc4ccccc45)cc(-c4cccc5c4oc4ccccc45)c3)n2)cc1 ; c1ccc(-c2nc(-c3ccc(-c4cncnc4)cc3)cc(-c3cc(-c4cccc5c4oc4ccccc45)cc(-c4cccc5c4oc4ccccc45)c3)n2)cc1]

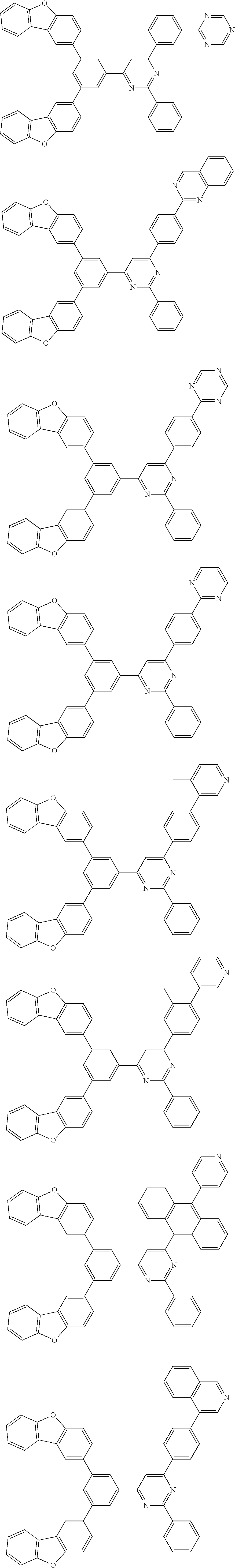

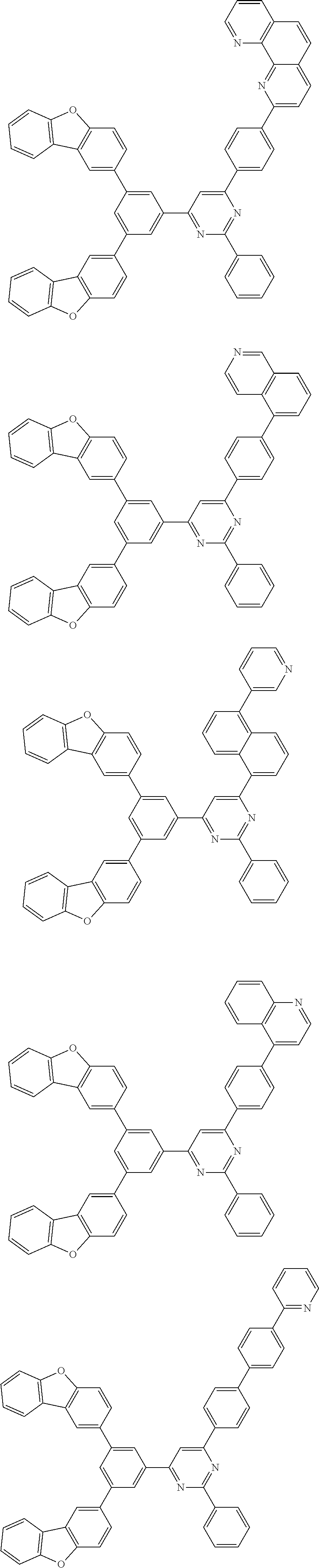

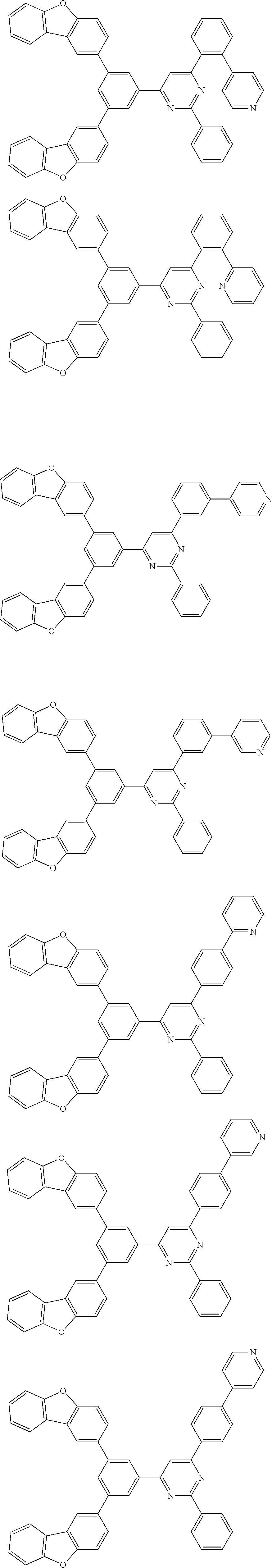

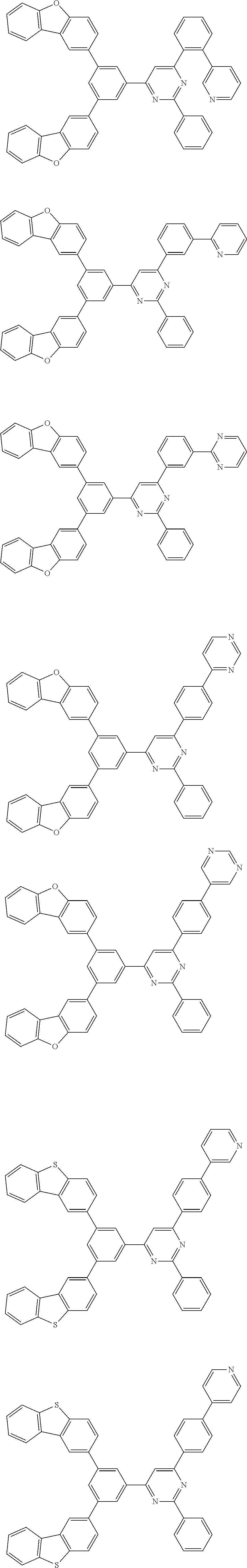

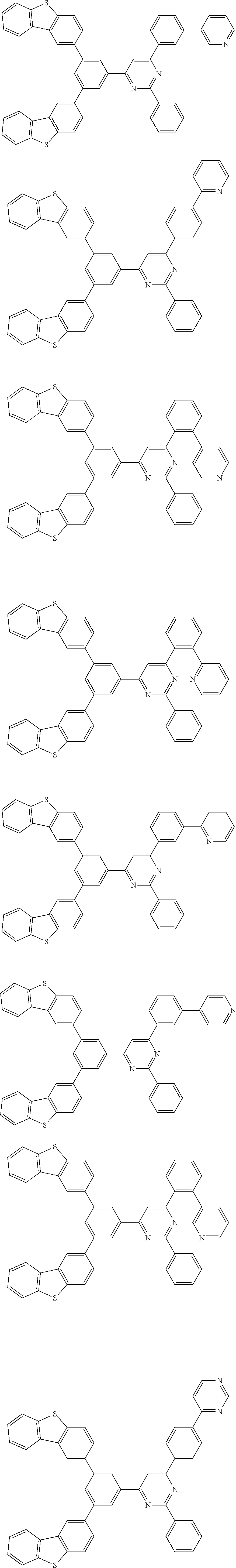

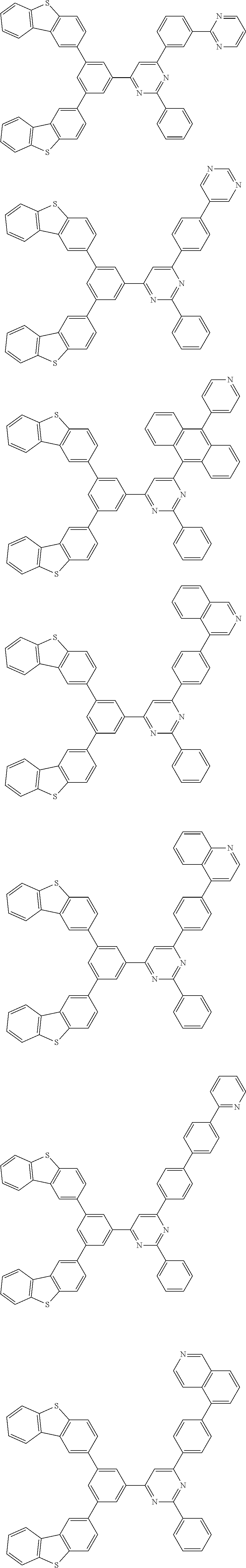

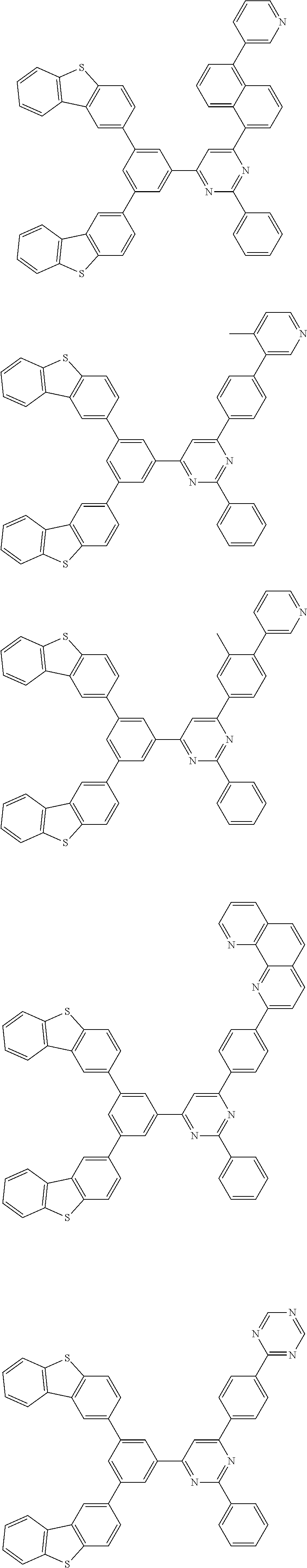

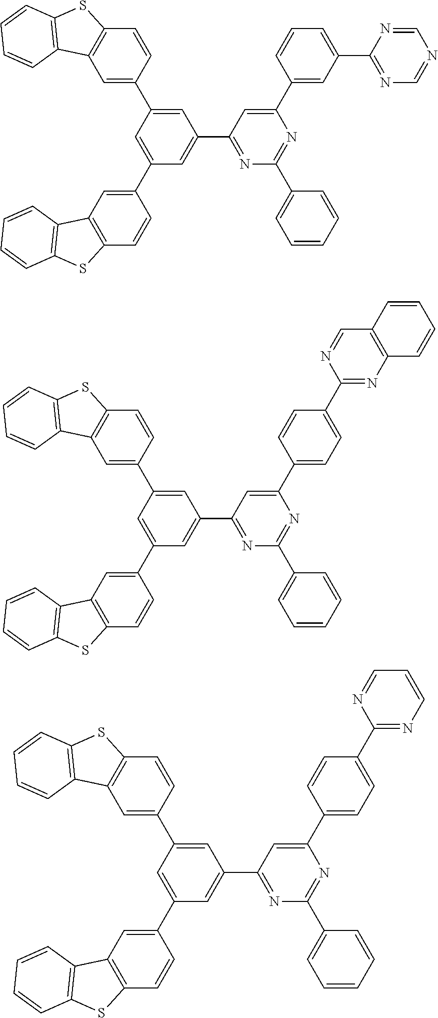 [SMILES: c1ccc(-c2nc(-c3cc(-c4ccc5sc6ccccc6c5c4)cc(-c4ccc5sc6ccccc6c5c4)c3)cc(-c3cccc(-c4ncncn4)c3)n2)cc1 ; c1ccc(-c2nc(-c3ccc(-c4ncc5ccccc5n4)cc3)cc(-c3cc(-c4ccc5sc6ccccc6c5c4)cc(-c4ccc5sc6ccccc6c5c4)c3)n2)cc1 ; c1ccc(-c2nc(-c3ccc(-c4ncccn4)cc3)cc(-c3cc(-c4ccc5sc6ccccc6c5c4)cc(-c4ccc5sc6ccccc6c5c4)c3)n2)cc1]

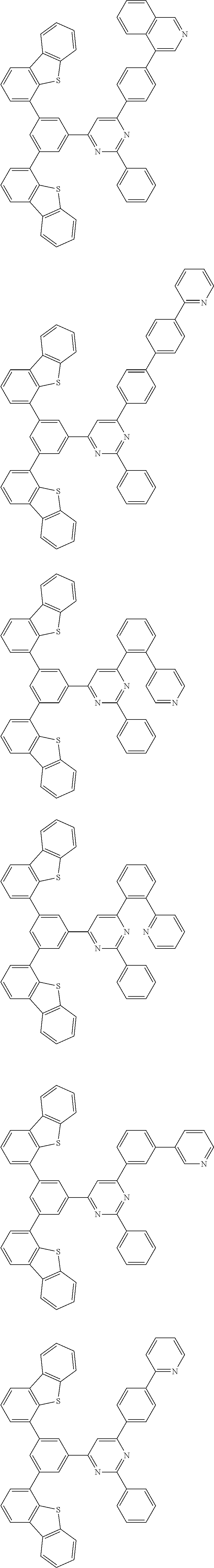

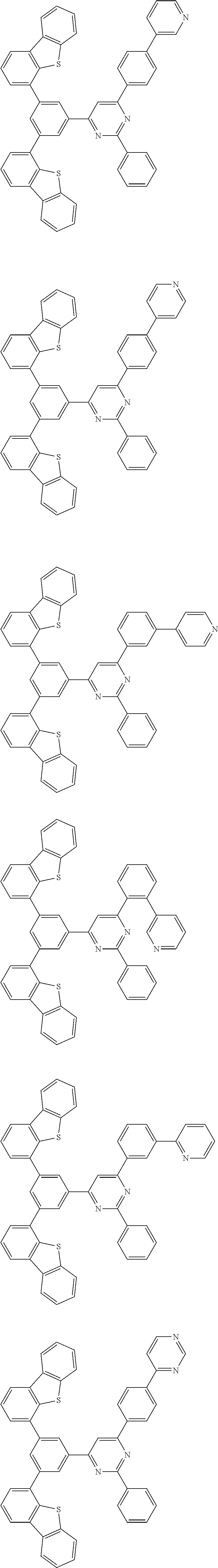

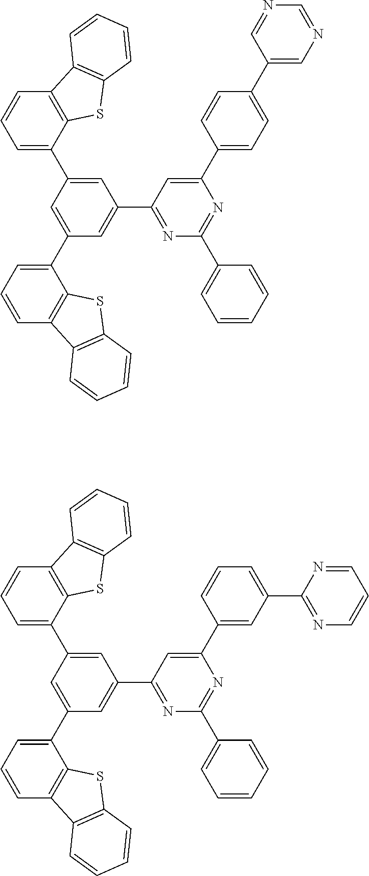 [SMILES: c1ccc(-c2nc(-c3ccc(-c4cncnc4)cc3)cc(-c3cc(-c4cccc5c4sc4ccccc45)cc(-c4cccc5c4sc4ccccc45)c3)n2)cc1 ; c1ccc(-c2nc(-c3cccc(-c4ncccn4)c3)cc(-c3cc(-c4cccc5c4sc4ccccc45)cc(-c4cccc5c4sc4ccccc45)c3)n2)cc1]

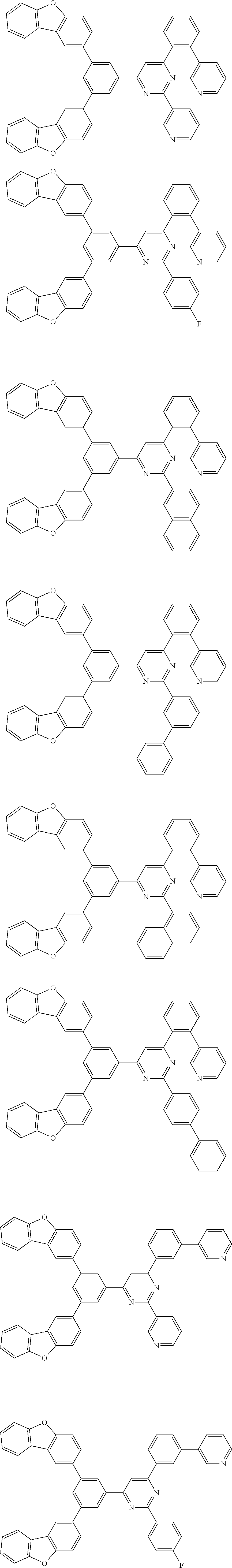

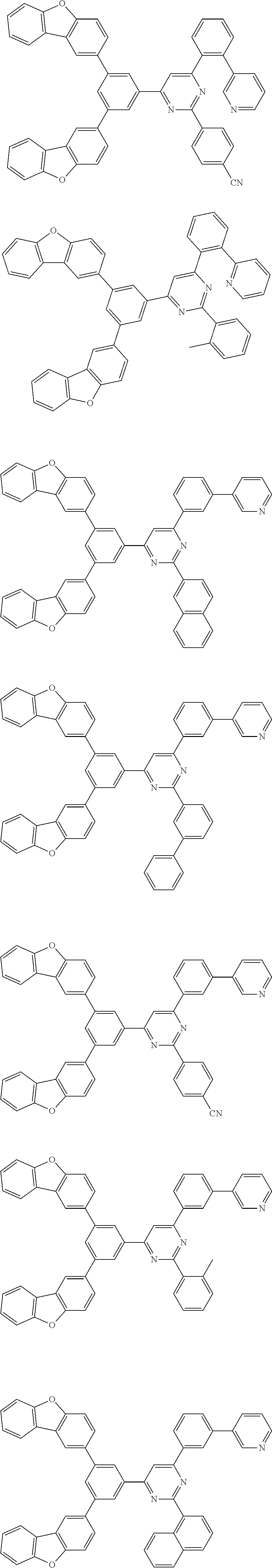

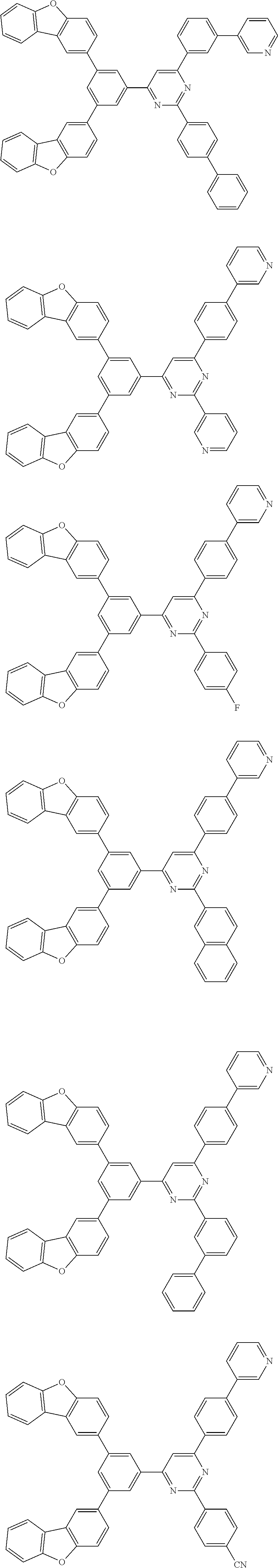

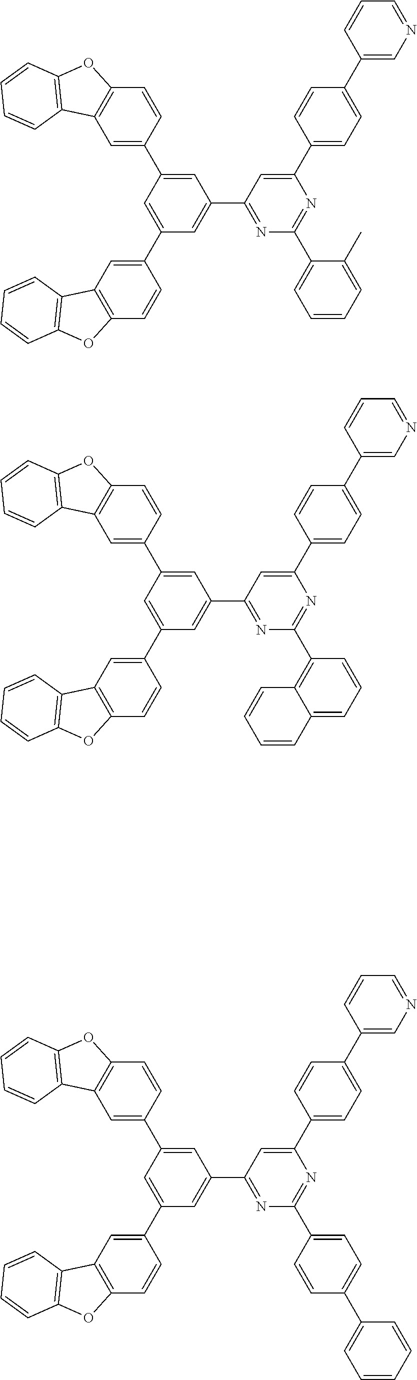 [SMILES: Cc1ccccc1-c1nc(-c2ccc(-c3cccnc3)cc2)cc(-c2cc(-c3ccc4oc5ccccc5c4c3)cc(-c3ccc4oc5ccccc5c4c3)c2)n1 ; c1cncc(-c2ccc(-c3cc(-c4cc(-c5ccc6oc7ccccc7c6c5)cc(-c5ccc6oc7ccccc7c6c5)c4)nc(-c4cccc5ccccc45)n3)cc2)c1 ; c1ccc(-c2ccc(-c3nc(-c4ccc(-c5cccnc5)cc4)cc(-c4cc(-c5ccc6oc7ccccc7c6c5)cc(-c5ccc6oc7ccccc7c6c5)c4)n3)cc2)cc1]

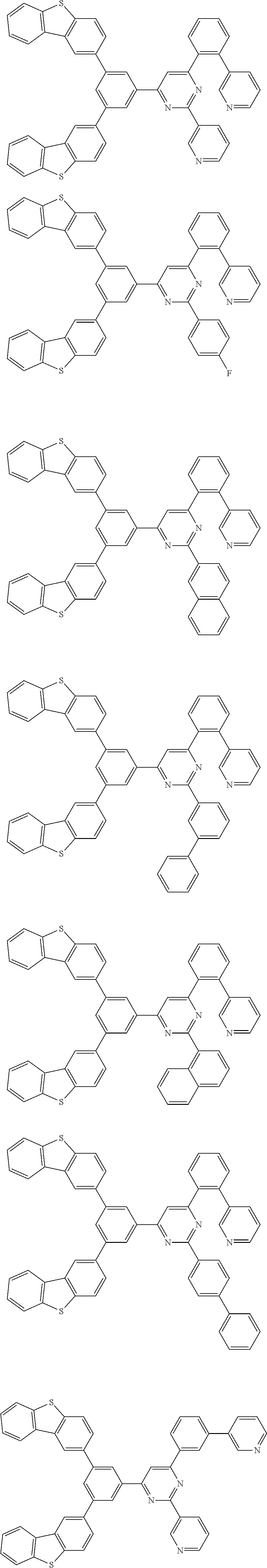

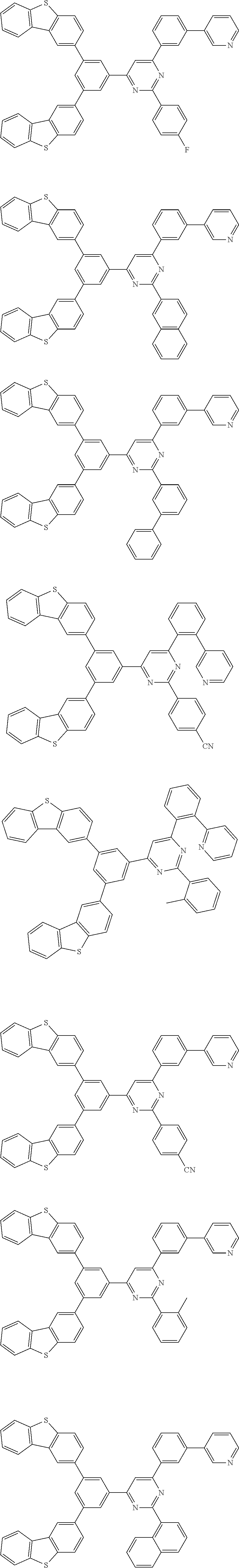

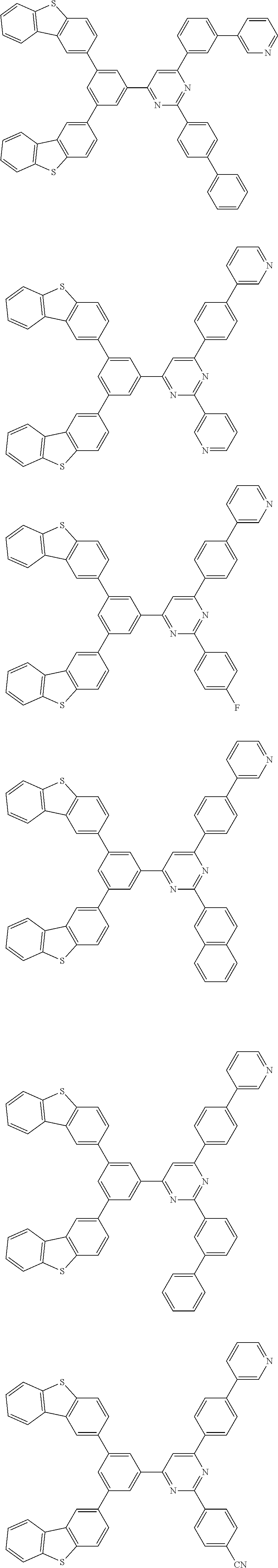

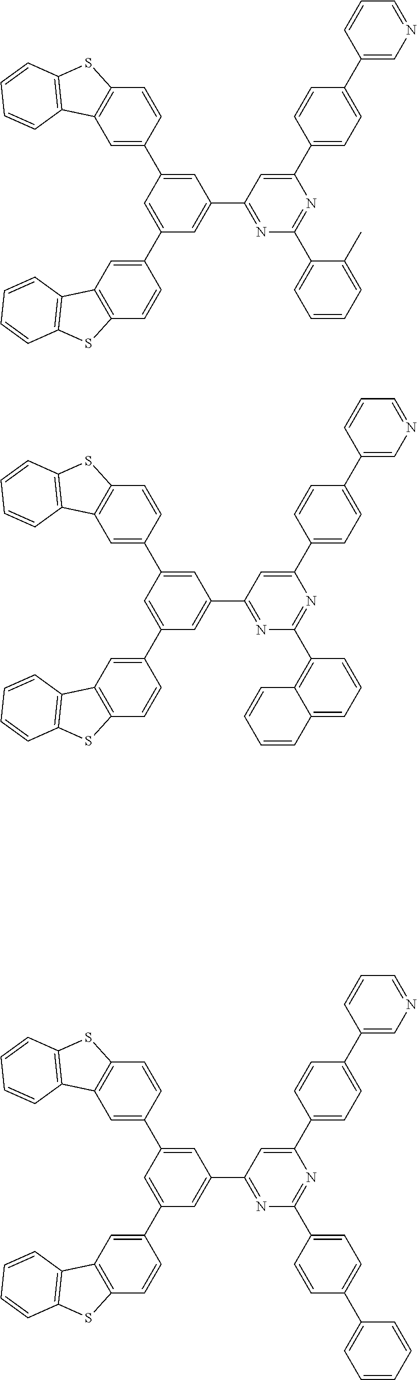 [SMILES: Cc1ccccc1-c1nc(-c2ccc(-c3cccnc3)cc2)cc(-c2cc(-c3ccc4sc5ccccc5c4c3)cc(-c3ccc4sc5ccccc5c4c3)c2)n1 ; c1cncc(-c2ccc(-c3cc(-c4cc(-c5ccc6sc7ccccc7c6c5)cc(-c5ccc6sc7ccccc7c6c5)c4)nc(-c4cccc5ccccc45)n3)cc2)c1 ; c1ccc(-c2ccc(-c3nc(-c4ccc(-c5cccnc5)cc4)cc(-c4cc(-c5ccc6sc7ccccc7c6c5)cc(-c5ccc6sc7ccccc7c6c5)c4)n3)cc2)cc1]

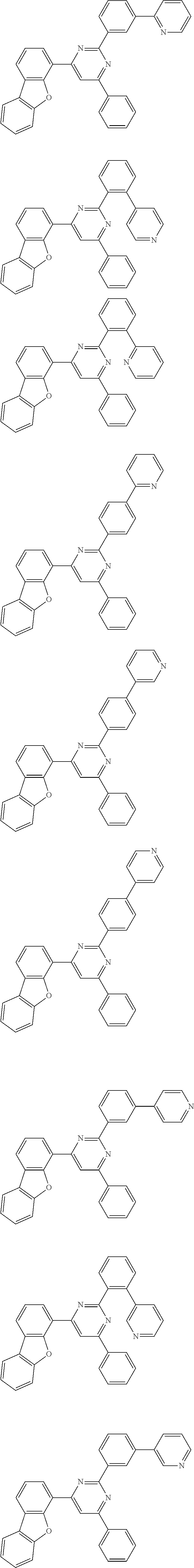

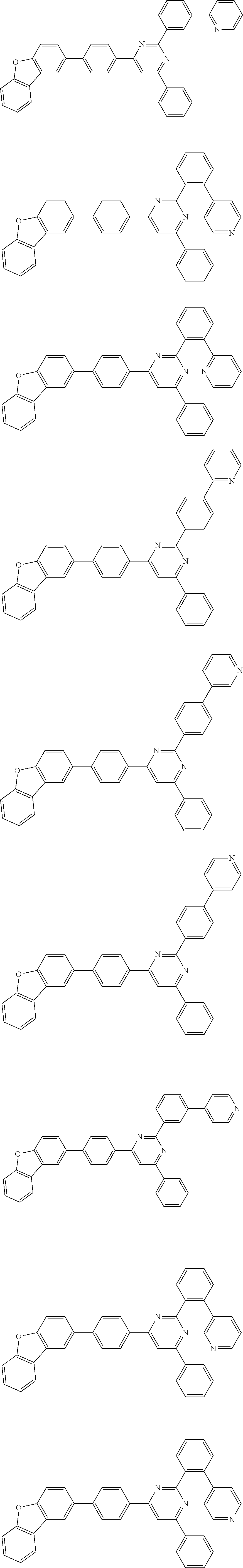

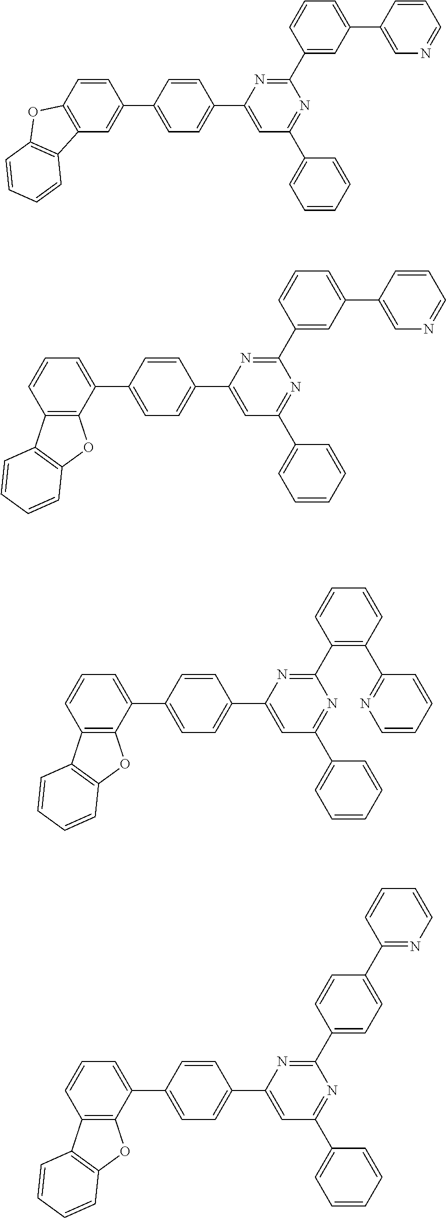 [SMILES: c1ccc(-c2cc(-c3ccc(-c4ccc5oc6ccccc6c5c4)cc3)nc(-c3cccc(-c4cccnc4)c3)n2)cc1 ; c1ccc(-c2cc(-c3ccc(-c4cccc5c4oc4ccccc45)cc3)nc(-c3cccc(-c4cccnc4)c3)n2)cc1 ; c1ccc(-c2cc(-c3ccc(-c4cccc5c4oc4ccccc45)cc3)nc(-c3ccccc3-c3ccccn3)n2)cc1 ; c1ccc(-c2cc(-c3ccc(-c4cccc5c4oc4ccccc45)cc3)nc(-c3ccc(-c4ccccn4)cc3)n2)cc1]

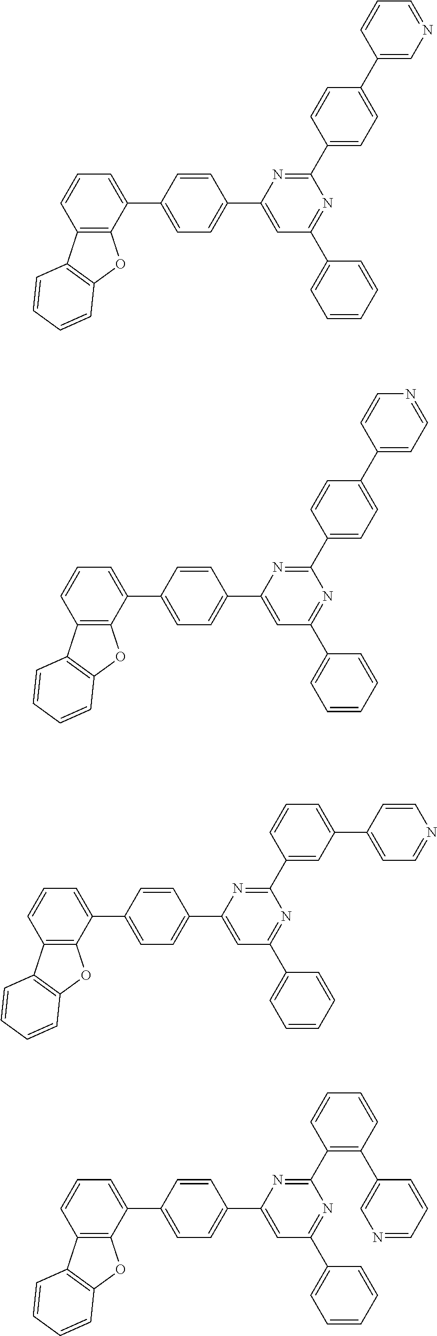 [SMILES: c1ccc(-c2cc(-c3ccc(-c4cccc5c4oc4ccccc45)cc3)nc(-c3ccc(-c4cccnc4)cc3)n2)cc1 ; c1ccc(-c2cc(-c3ccc(-c4cccc5c4oc4ccccc45)cc3)nc(-c3ccc(-c4ccncc4)cc3)n2)cc1 ; c1ccc(-c2cc(-c3ccc(-c4cccc5c4oc4ccccc45)cc3)nc(-c3cccc(-c4ccncc4)c3)n2)cc1 ; c1ccc(-c2cc(-c3ccc(-c4cccc5c4oc4ccccc45)cc3)nc(-c3ccccc3-c3cccnc3)n2)cc1]

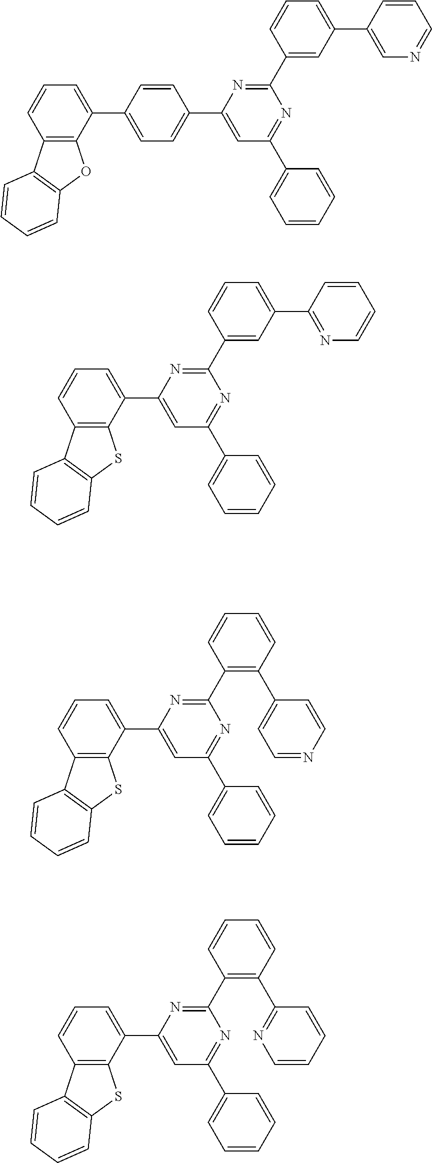 [SMILES: c1ccc(-c2cc(-c3ccc(-c4cccc5c4oc4ccccc45)cc3)nc(-c3cccc(-c4cccnc4)c3)n2)cc1 ; c1ccc(-c2cc(-c3cccc4c3sc3ccccc34)nc(-c3cccc(-c4ccccn4)c3)n2)cc1 ; c1ccc(-c2cc(-c3cccc4c3sc3ccccc34)nc(-c3ccccc3-c3ccncc3)n2)cc1 ; c1ccc(-c2cc(-c3cccc4c3sc3ccccc34)nc(-c3ccccc3-c3ccccn3)n2)cc1]

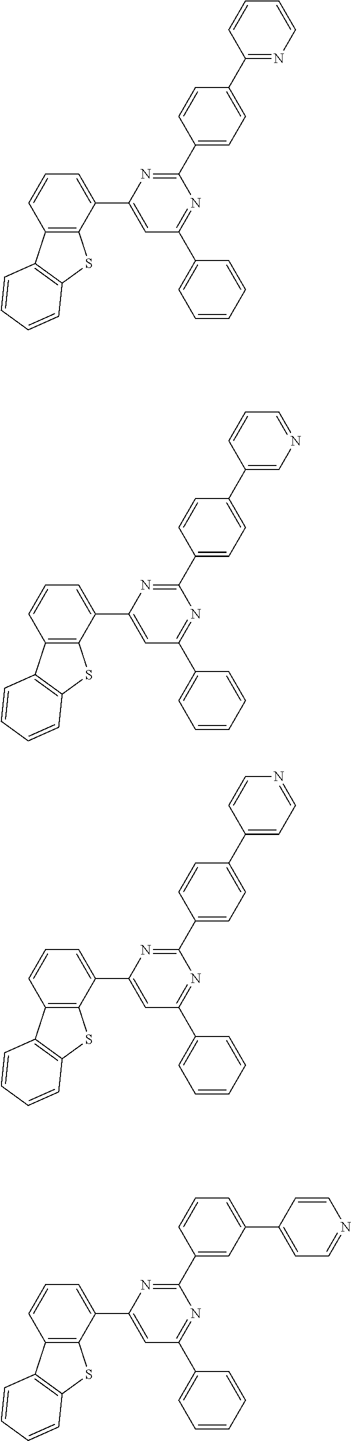 [SMILES: c1ccc(-c2cc(-c3cccc4c3sc3ccccc34)nc(-c3ccc(-c4ccccn4)cc3)n2)cc1 ; c1ccc(-c2cc(-c3cccc4c3sc3ccccc34)nc(-c3ccc(-c4cccnc4)cc3)n2)cc1 ; c1ccc(-c2cc(-c3cccc4c3sc3ccccc34)nc(-c3ccc(-c4ccncc4)cc3)n2)cc1 ; c1ccc(-c2cc(-c3cccc4c3sc3ccccc34)nc(-c3cccc(-c4ccncc4)c3)n2)cc1]

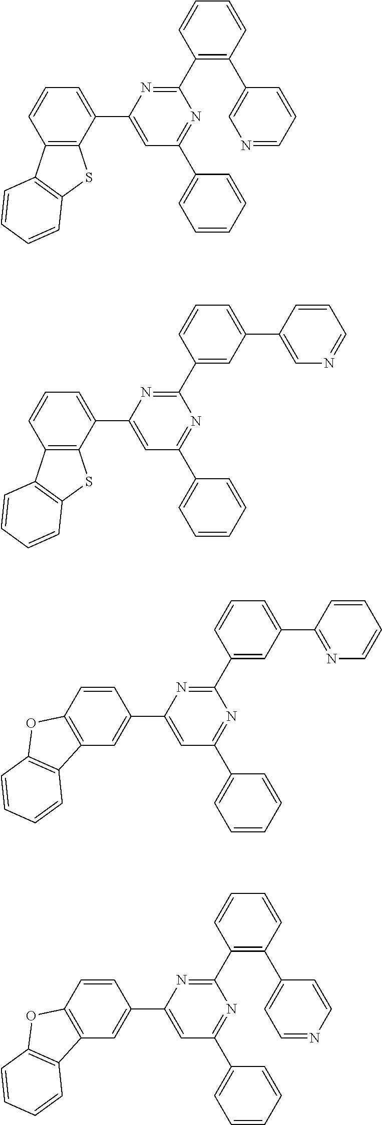 [SMILES: c1ccc(-c2cc(-c3cccc4c3sc3ccccc34)nc(-c3ccccc3-c3cccnc3)n2)cc1 ; c1ccc(-c2cc(-c3cccc4c3sc3ccccc34)nc(-c3cccc(-c4cccnc4)c3)n2)cc1 ; c1ccc(-c2cc(-c3ccc4oc5ccccc5c4c3)nc(-c3cccc(-c4ccccn4)c3)n2)cc1 ; c1ccc(-c2cc(-c3ccc4oc5ccccc5c4c3)nc(-c3ccccc3-c3ccncc3)n2)cc1]

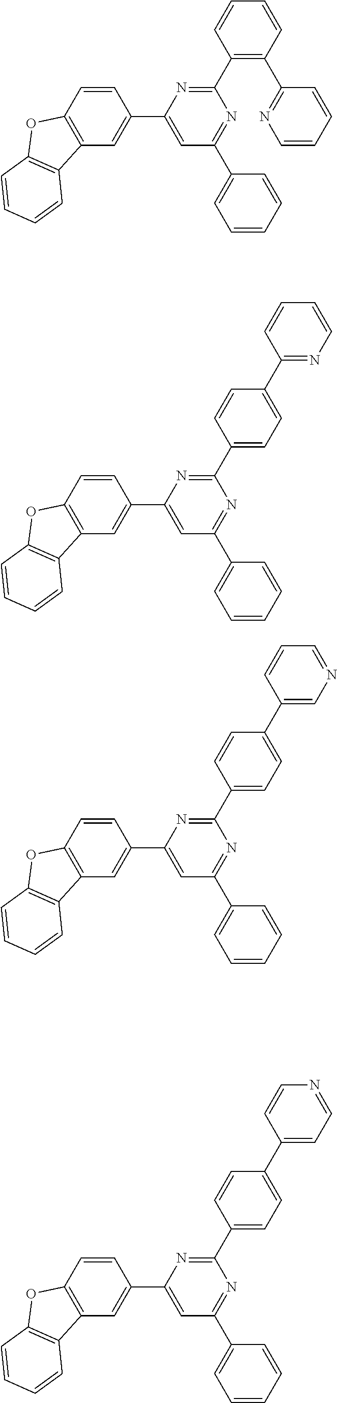

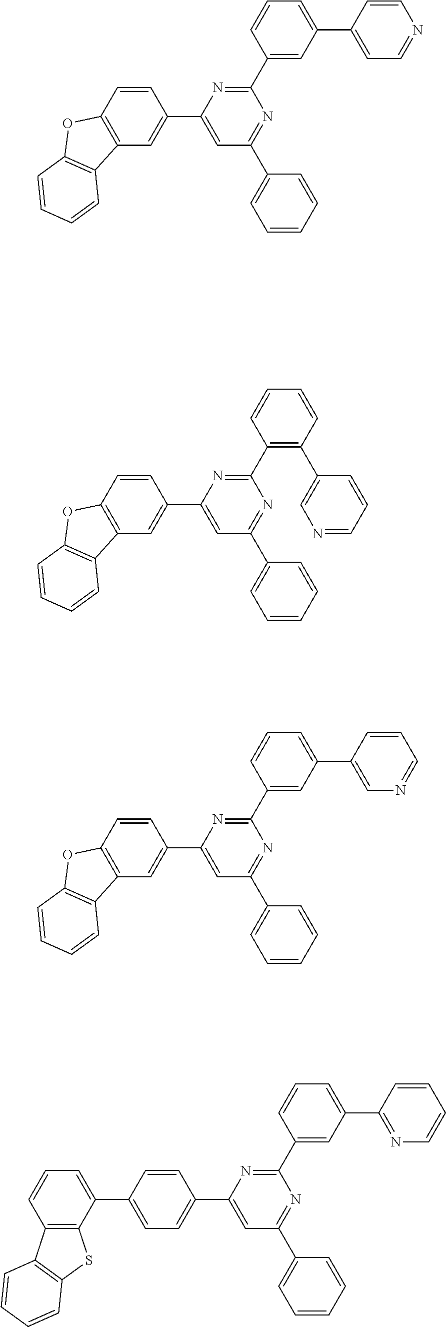 [SMILES: c1ccc(-c2cc(-c3ccc4oc5ccccc5c4c3)nc(-c3cccc(-c4ccncc4)c3)n2)cc1 ; c1ccc(-c2cc(-c3ccc4oc5ccccc5c4c3)nc(-c3ccccc3-c3cccnc3)n2)cc1 ; c1ccc(-c2cc(-c3ccc4oc5ccccc5c4c3)nc(-c3cccc(-c4cccnc4)c3)n2)cc1 ; c1ccc(-c2cc(-c3ccc(-c4cccc5c4sc4ccccc45)cc3)nc(-c3cccc(-c4ccccn4)c3)n2)cc1]

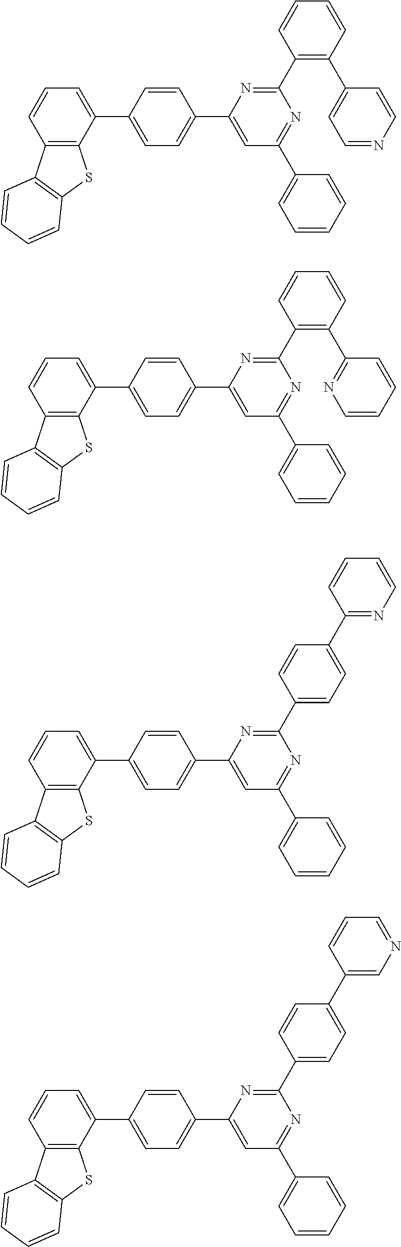

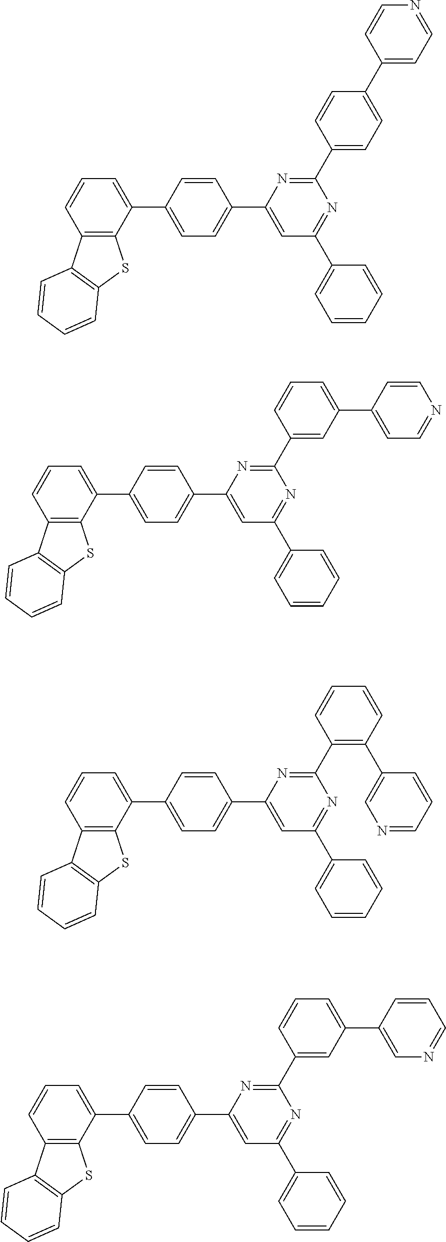 [SMILES: c1ccc(-c2cc(-c3ccc(-c4cccc5c4sc4ccccc45)cc3)nc(-c3ccc(-c4ccncc4)cc3)n2)cc1 ; c1ccc(-c2cc(-c3ccc(-c4cccc5c4sc4ccccc45)cc3)nc(-c3cccc(-c4ccncc4)c3)n2)cc1 ; c1ccc(-c2cc(-c3ccc(-c4cccc5c4sc4ccccc45)cc3)nc(-c3ccccc3-c3cccnc3)n2)cc1 ; c1ccc(-c2cc(-c3ccc(-c4cccc5c4sc4ccccc45)cc3)nc(-c3cccc(-c4cccnc4)c3)n2)cc1]

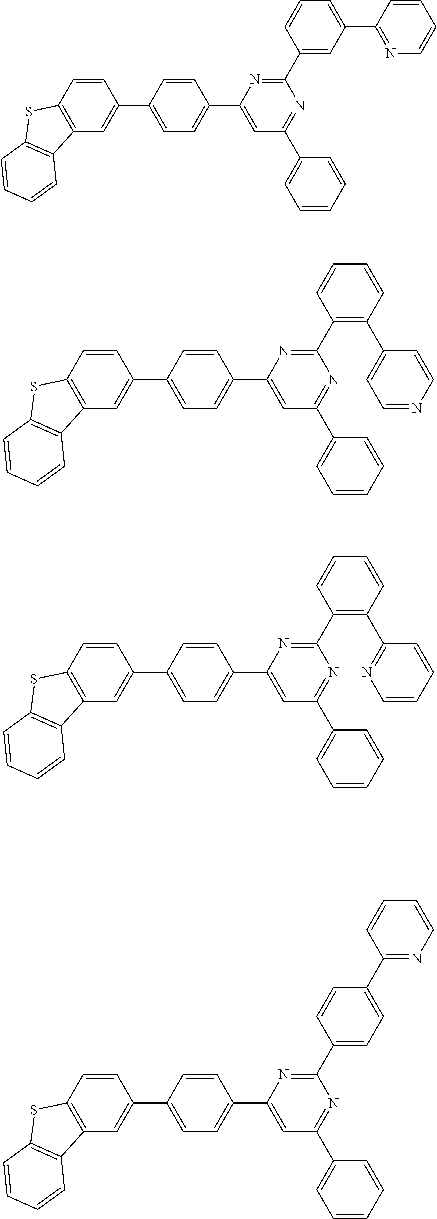 [SMILES: c1ccc(-c2cc(-c3ccc(-c4ccc5sc6ccccc6c5c4)cc3)nc(-c3cccc(-c4ccccn4)c3)n2)cc1 ; c1ccc(-c2cc(-c3ccc(-c4ccc5sc6ccccc6c5c4)cc3)nc(-c3ccccc3-c3ccncc3)n2)cc1 ; c1ccc(-c2cc(-c3ccc(-c4ccc5sc6ccccc6c5c4)cc3)nc(-c3ccccc3-c3ccccn3)n2)cc1 ; c1ccc(-c2cc(-c3ccc(-c4ccc5sc6ccccc6c5c4)cc3)nc(-c3ccc(-c4ccccn4)cc3)n2)cc1]

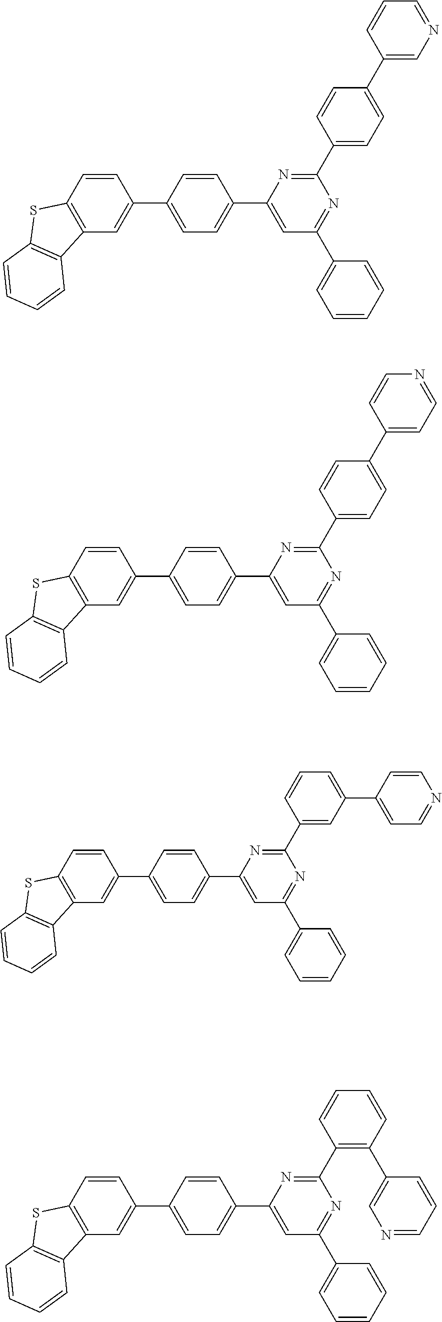 [SMILES: c1ccc(-c2cc(-c3ccc(-c4ccc5sc6ccccc6c5c4)cc3)nc(-c3ccc(-c4cccnc4)cc3)n2)cc1 ; c1ccc(-c2cc(-c3ccc(-c4ccc5sc6ccccc6c5c4)cc3)nc(-c3ccc(-c4ccncc4)cc3)n2)cc1 ; c1ccc(-c2cc(-c3ccc(-c4ccc5sc6ccccc6c5c4)cc3)nc(-c3cccc(-c4ccncc4)c3)n2)cc1 ; c1ccc(-c2cc(-c3ccc(-c4ccc5sc6ccccc6c5c4)cc3)nc(-c3ccccc3-c3cccnc3)n2)cc1]

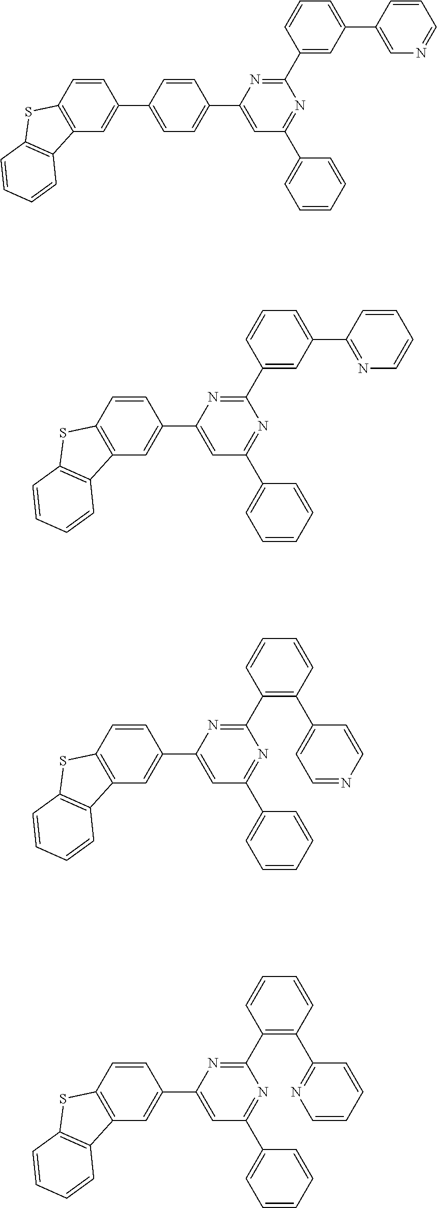 [SMILES: c1ccc(-c2cc(-c3ccc(-c4ccc5sc6ccccc6c5c4)cc3)nc(-c3cccc(-c4cccnc4)c3)n2)cc1 ; c1ccc(-c2cc(-c3ccc4sc5ccccc5c4c3)nc(-c3cccc(-c4ccccn4)c3)n2)cc1 ; c1ccc(-c2cc(-c3ccc4sc5ccccc5c4c3)nc(-c3ccccc3-c3ccncc3)n2)cc1 ; c1ccc(-c2cc(-c3ccc4sc5ccccc5c4c3)nc(-c3ccccc3-c3ccccn3)n2)cc1]

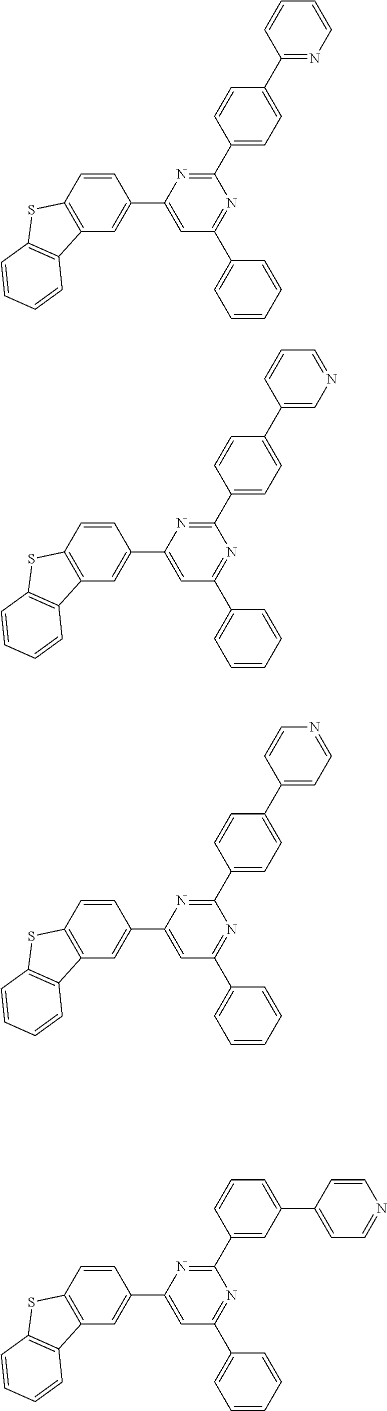

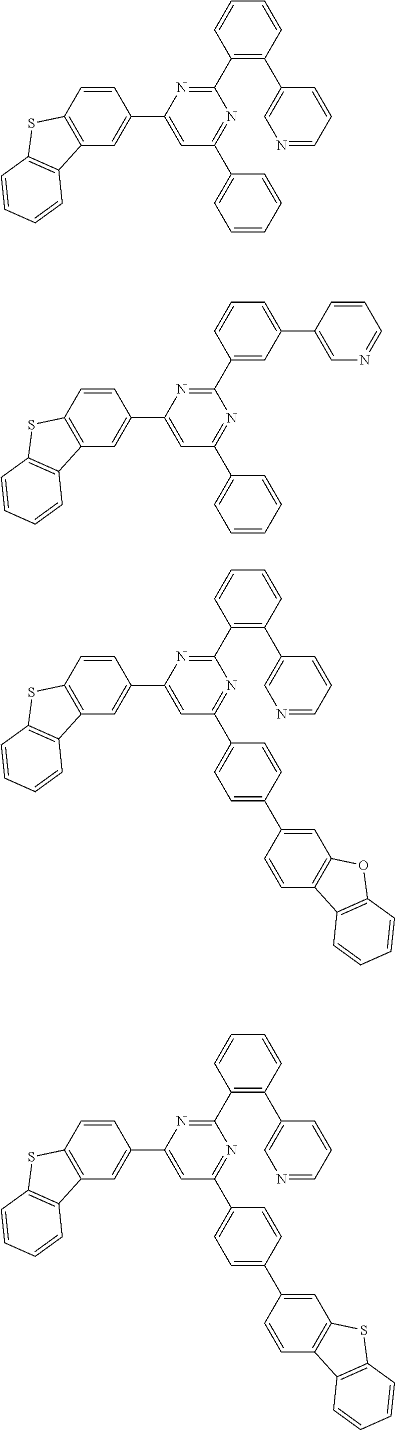

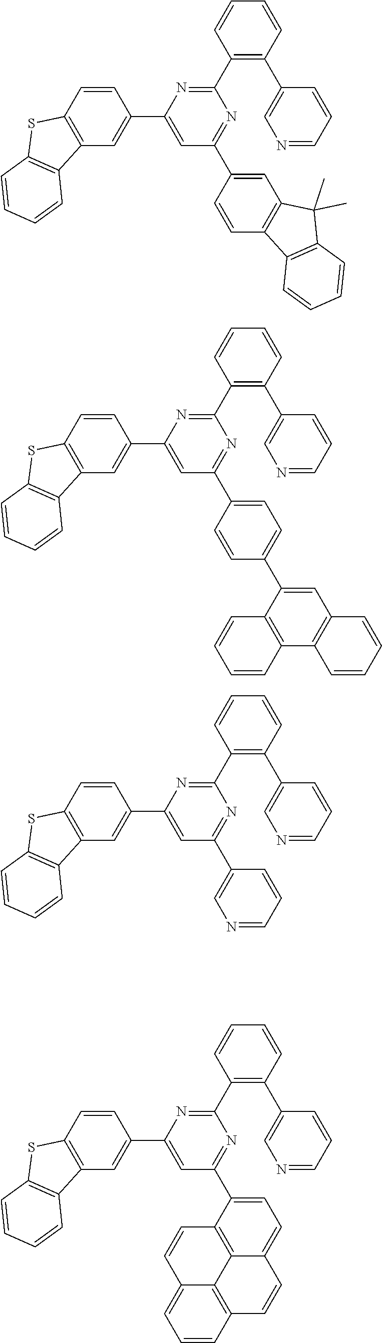

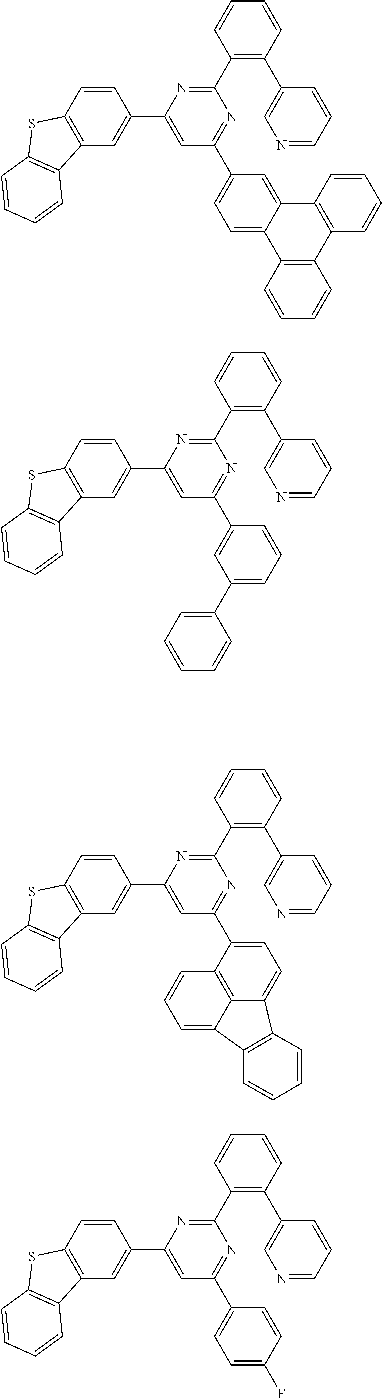

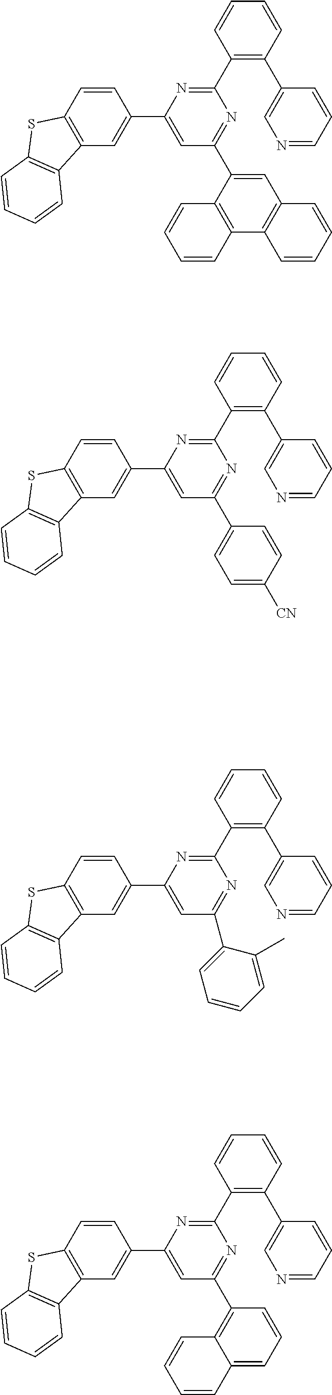 [SMILES: c1cncc(-c2ccccc2-c2nc(-c3ccc4sc5ccccc5c4c3)cc(-c3cc4ccccc4c4ccccc34)n2)c1 ; N#Cc1ccc(-c2cc(-c3ccc4sc5ccccc5c4c3)nc(-c3ccccc3-c3cccnc3)n2)cc1 ; Cc1ccccc1-c1cc(-c2ccc3sc4ccccc4c3c2)nc(-c2ccccc2-c2cccnc2)n1 ; c1cncc(-c2ccccc2-c2nc(-c3ccc4sc5ccccc5c4c3)cc(-c3cccc4ccccc34)n2)c1]

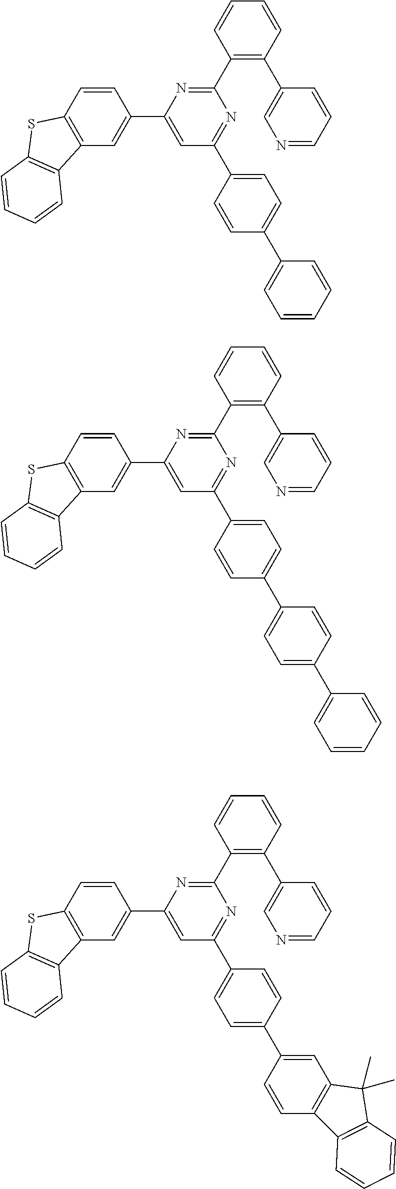

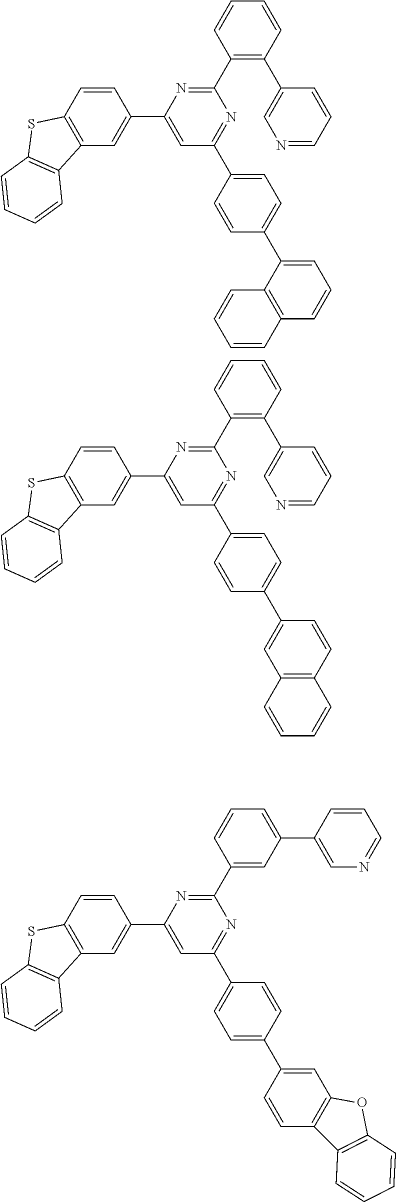

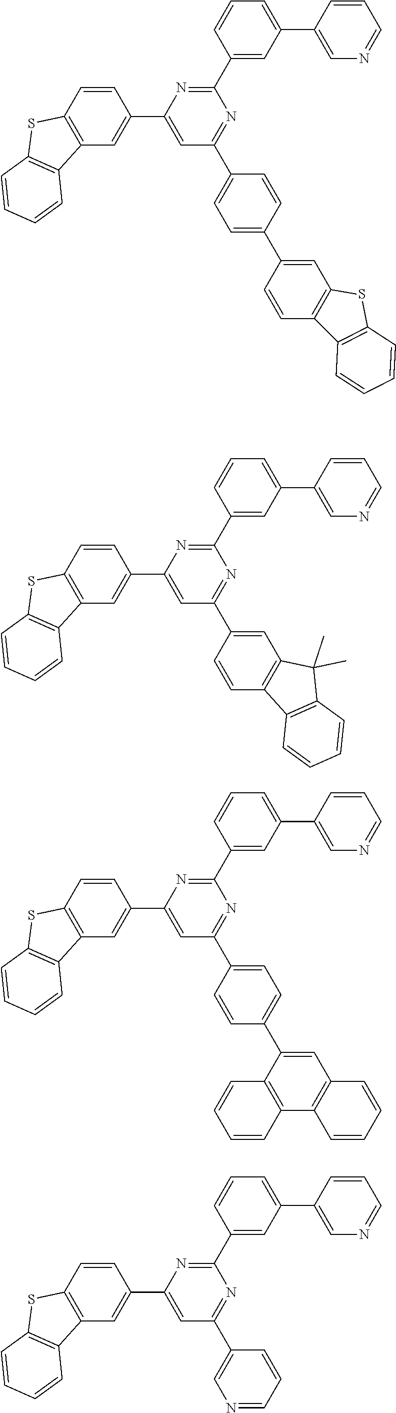

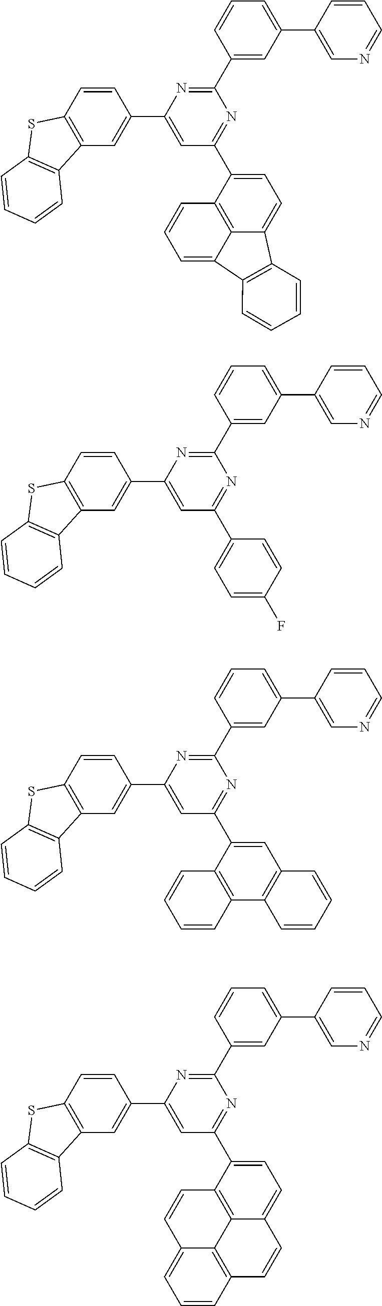

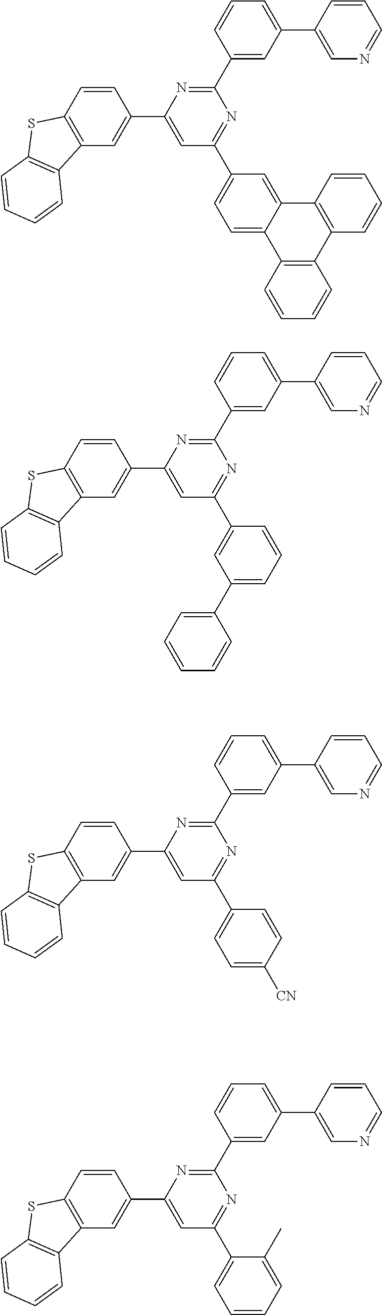

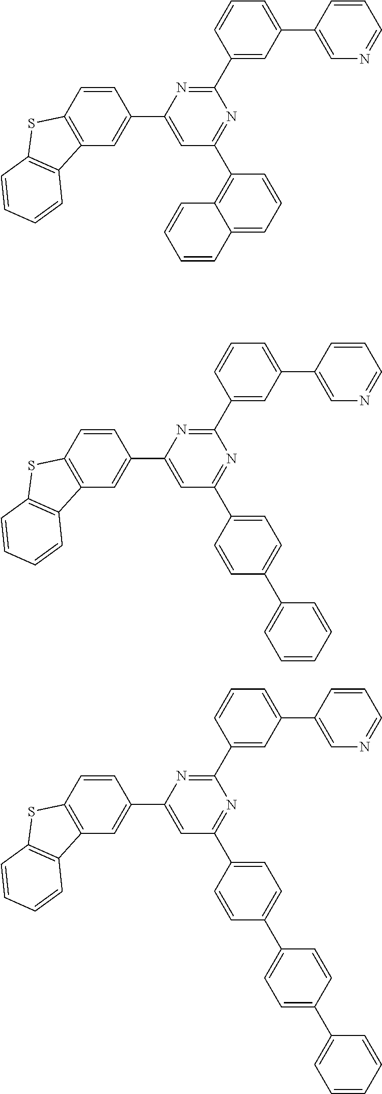

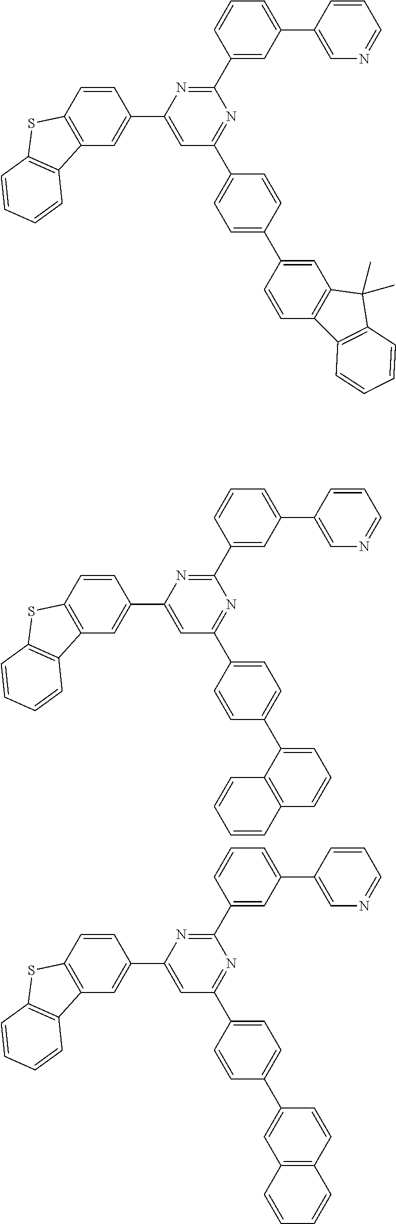

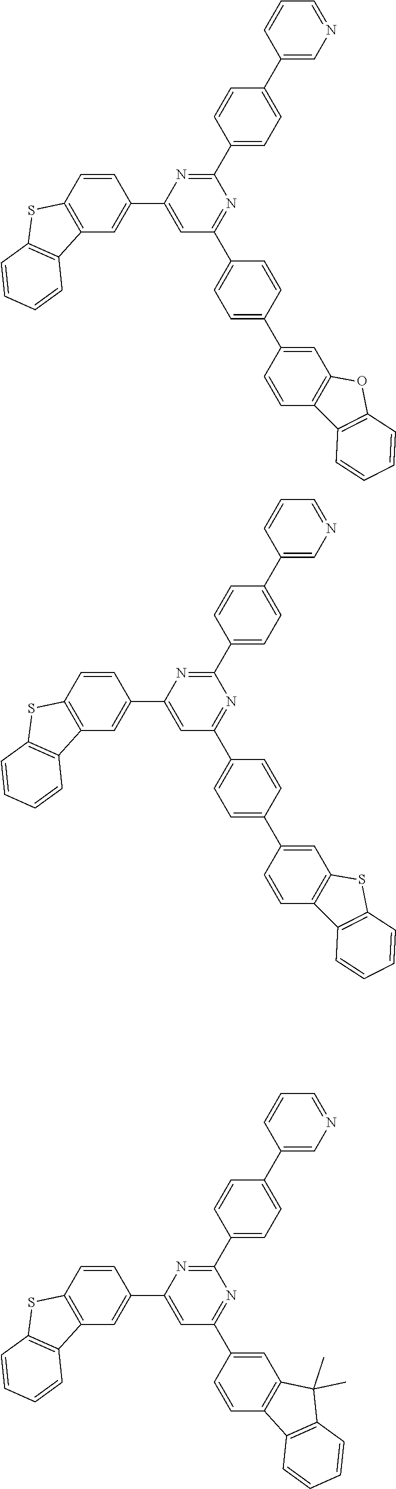

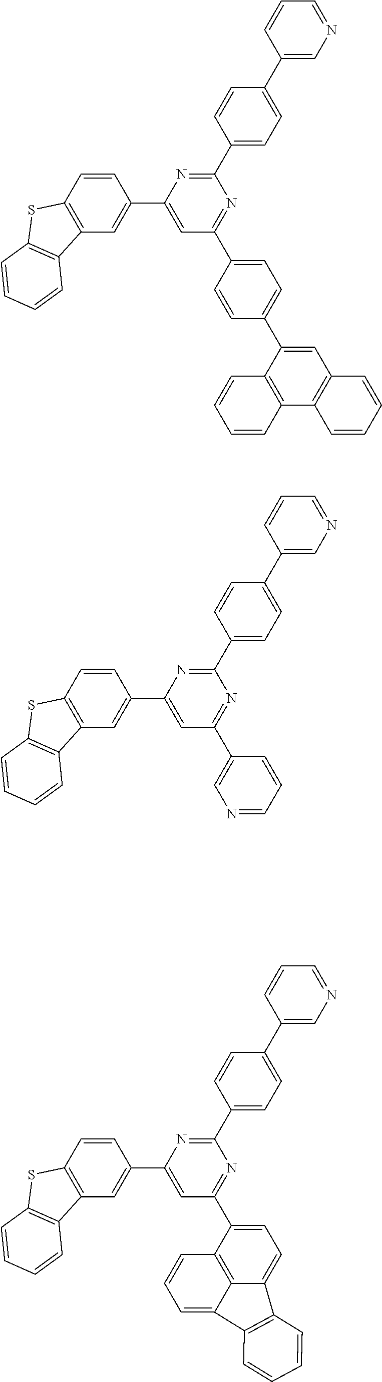 [SMILES: c1cncc(-c2ccc(-c3nc(-c4ccc(-c5cc6ccccc6c6ccccc56)cc4)cc(-c4ccc5sc6ccccc6c5c4)n3)cc2)c1 ; c1cncc(-c2ccc(-c3nc(-c4cccnc4)cc(-c4ccc5sc6ccccc6c5c4)n3)cc2)c1 ; c1cncc(-c2ccc(-c3nc(-c4ccc5sc6ccccc6c5c4)cc(-c4ccc5c6c(cccc46)-c4ccccc4-5)n3)cc2)c1]

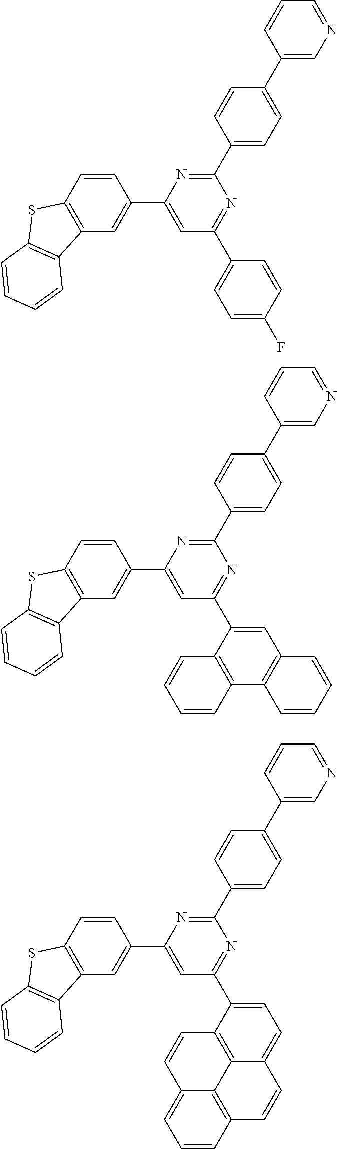

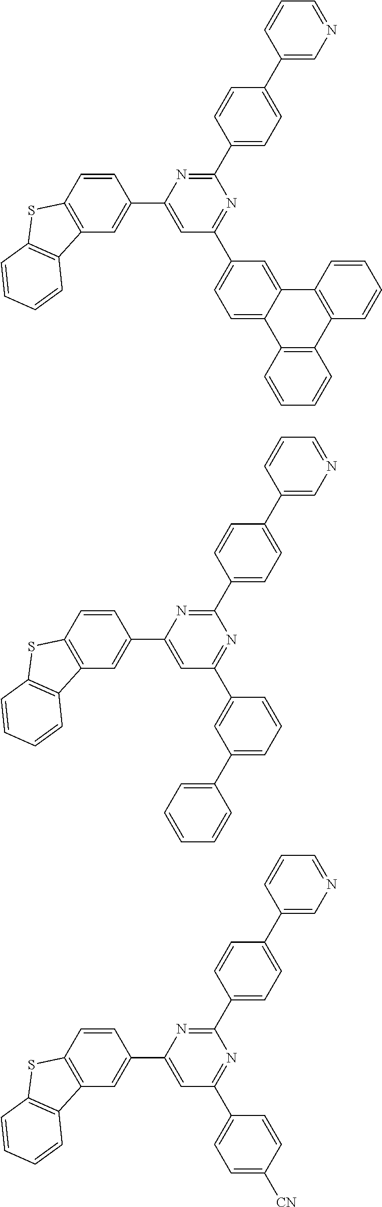

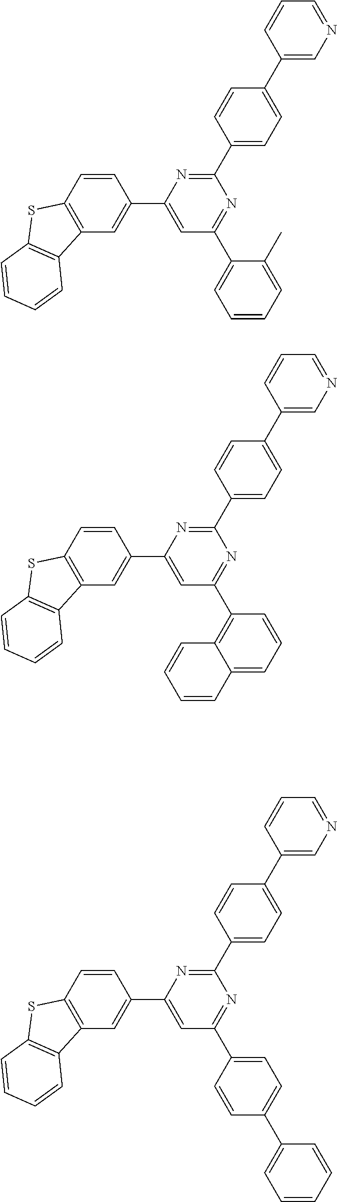

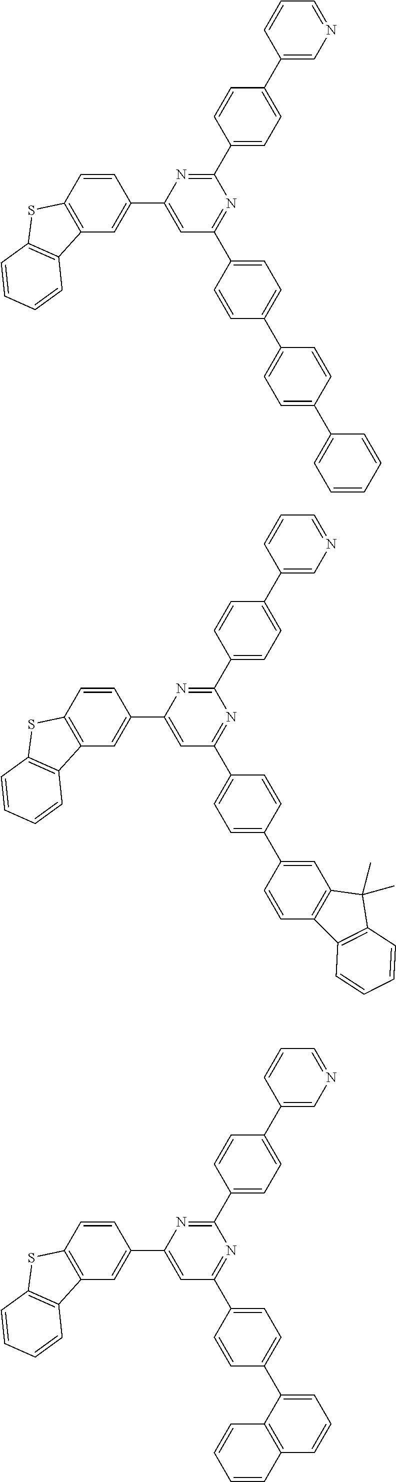

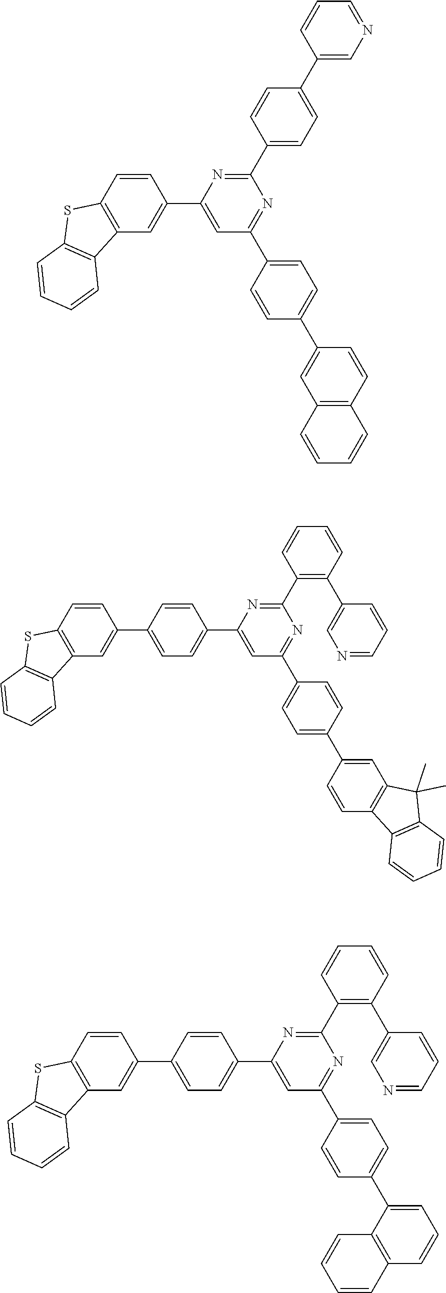 [SMILES: c1cncc(-c2ccc(-c3nc(-c4ccc(-c5ccc6ccccc6c5)cc4)cc(-c4ccc5sc6ccccc6c5c4)n3)cc2)c1 ; CC1(C)c2ccccc2-c2ccc(-c3ccc(-c4cc(-c5ccc(-c6ccc7sc8ccccc8c7c6)cc5)nc(-c5ccccc5-c5cccnc5)n4)cc3)cc21 ; c1cncc(-c2ccccc2-c2nc(-c3ccc(-c4ccc5sc6ccccc6c5c4)cc3)cc(-c3ccc(-c4cccc5ccccc45)cc3)n2)c1]

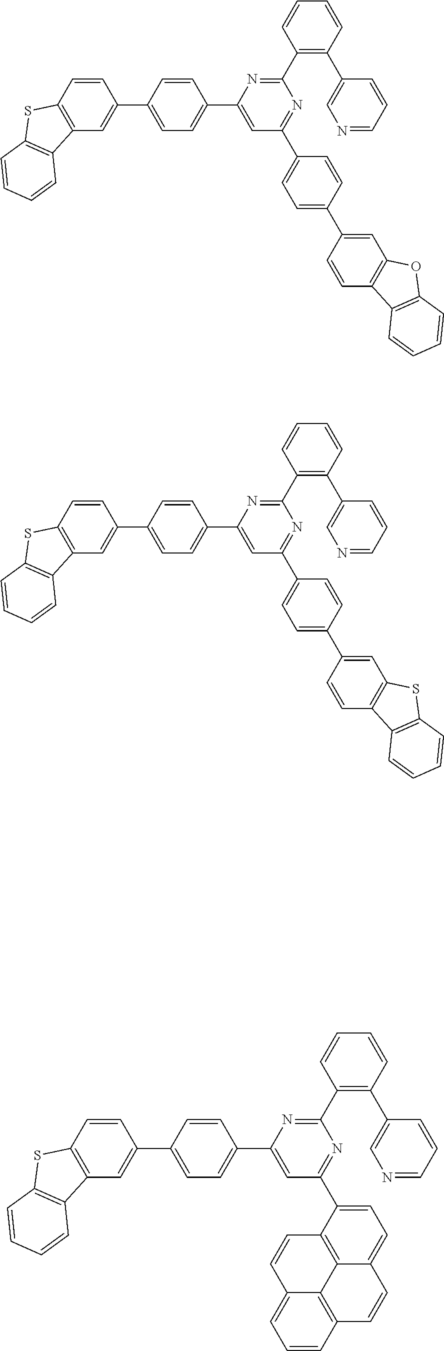 [SMILES: c1cncc(-c2ccccc2-c2nc(-c3ccc(-c4ccc5c(c4)oc4ccccc45)cc3)cc(-c3ccc(-c4ccc5sc6ccccc6c5c4)cc3)n2)c1 ; c1cncc(-c2ccccc2-c2nc(-c3ccc(-c4ccc5c(c4)sc4ccccc45)cc3)cc(-c3ccc(-c4ccc5sc6ccccc6c5c4)cc3)n2)c1 ; c1cncc(-c2ccccc2-c2nc(-c3ccc(-c4ccc5sc6ccccc6c5c4)cc3)cc(-c3ccc4ccc5cccc6ccc3c4c56)n2)c1]

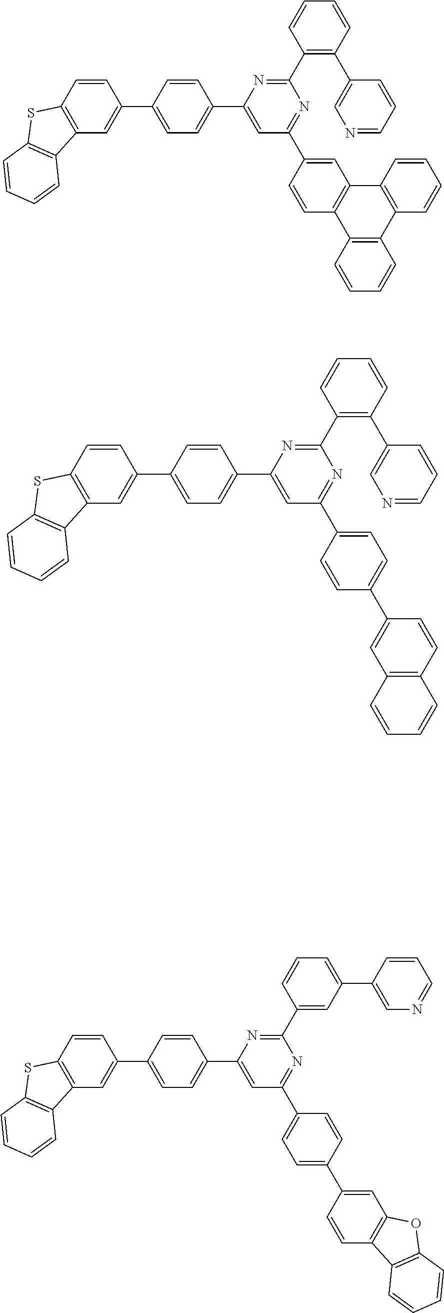 [SMILES: c1cncc(-c2ccccc2-c2nc(-c3ccc(-c4ccc5sc6ccccc6c5c4)cc3)cc(-c3ccc4c5ccccc5c5ccccc5c4c3)n2)c1 ; c1cncc(-c2ccccc2-c2nc(-c3ccc(-c4ccc5ccccc5c4)cc3)cc(-c3ccc(-c4ccc5sc6ccccc6c5c4)cc3)n2)c1 ; c1cncc(-c2cccc(-c3nc(-c4ccc(-c5ccc6c(c5)oc5ccccc56)cc4)cc(-c4ccc(-c5ccc6sc7ccccc7c6c5)cc4)n3)c2)c1]

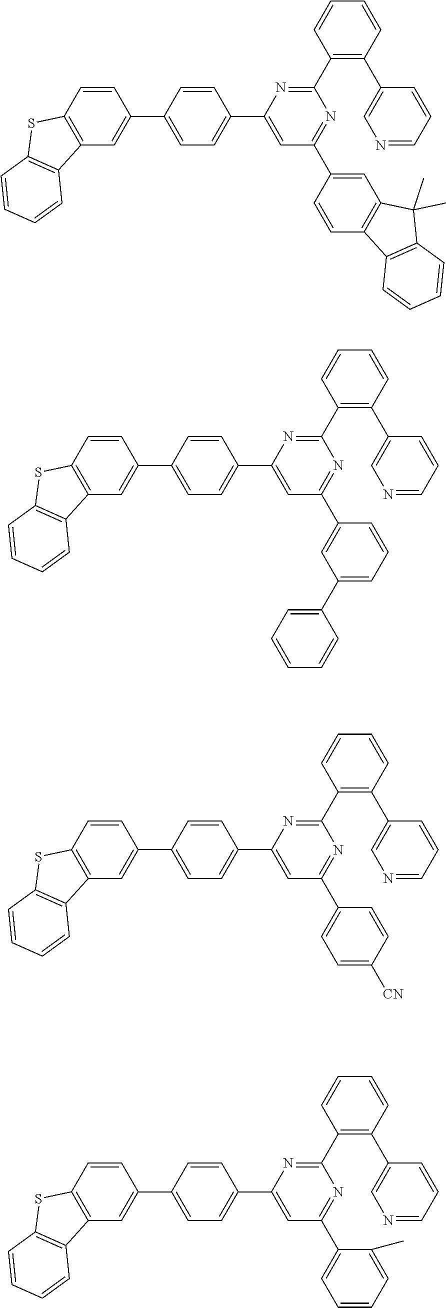 [SMILES: CC1(C)c2ccccc2-c2ccc(-c3cc(-c4ccc(-c5ccc6sc7ccccc7c6c5)cc4)nc(-c4ccccc4-c4cccnc4)n3)cc21 ; c1ccc(-c2cccc(-c3cc(-c4ccc(-c5ccc6sc7ccccc7c6c5)cc4)nc(-c4ccccc4-c4cccnc4)n3)c2)cc1 ; N#Cc1ccc(-c2cc(-c3ccc(-c4ccc5sc6ccccc6c5c4)cc3)nc(-c3ccccc3-c3cccnc3)n2)cc1 ; Cc1ccccc1-c1cc(-c2ccc(-c3ccc4sc5ccccc5c4c3)cc2)nc(-c2ccccc2-c2cccnc2)n1]

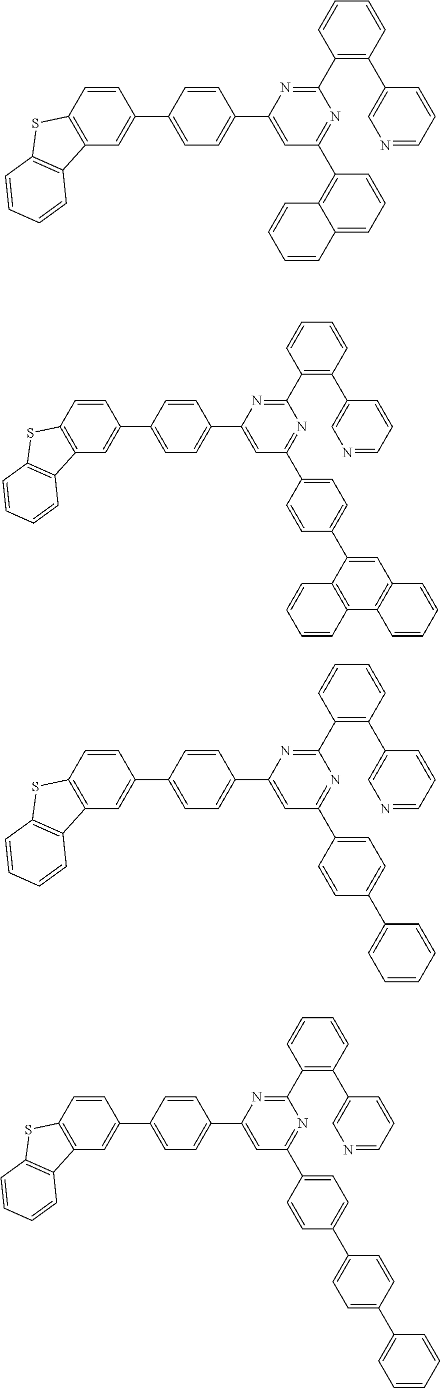

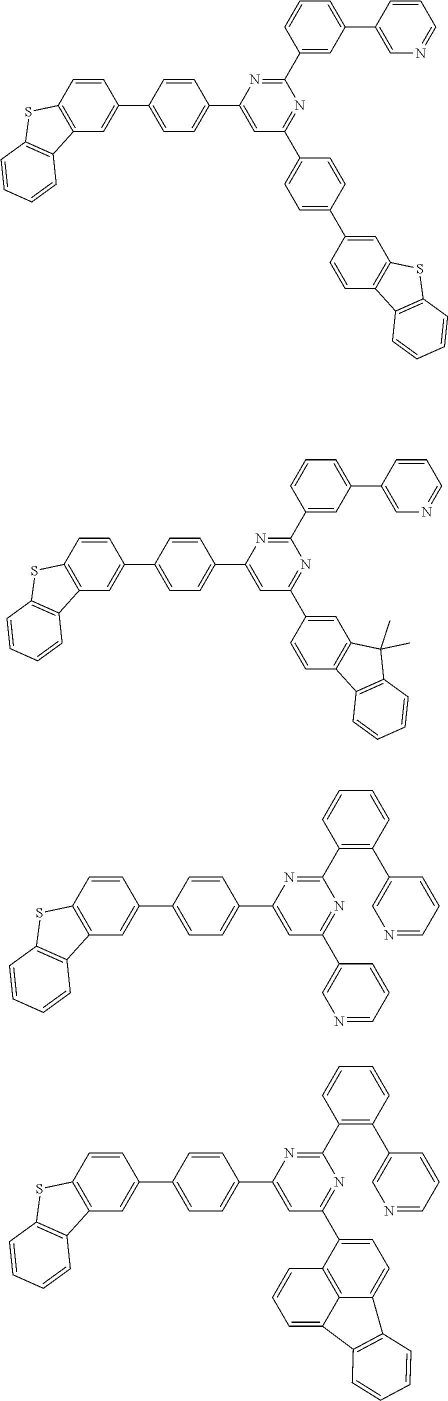 [SMILES: c1cncc(-c2cccc(-c3nc(-c4ccc(-c5ccc6c(c5)sc5ccccc56)cc4)cc(-c4ccc(-c5ccc6sc7ccccc7c6c5)cc4)n3)c2)c1 ; CC1(C)c2ccccc2-c2ccc(-c3cc(-c4ccc(-c5ccc6sc7ccccc7c6c5)cc4)nc(-c4cccc(-c5cccnc5)c4)n3)cc21 ; c1cncc(-c2cc(-c3ccc(-c4ccc5sc6ccccc6c5c4)cc3)nc(-c3ccccc3-c3cccnc3)n2)c1 ; c1cncc(-c2ccccc2-c2nc(-c3ccc(-c4ccc5sc6ccccc6c5c4)cc3)cc(-c3ccc4c5c(cccc35)-c3ccccc3-4)n2)c1]

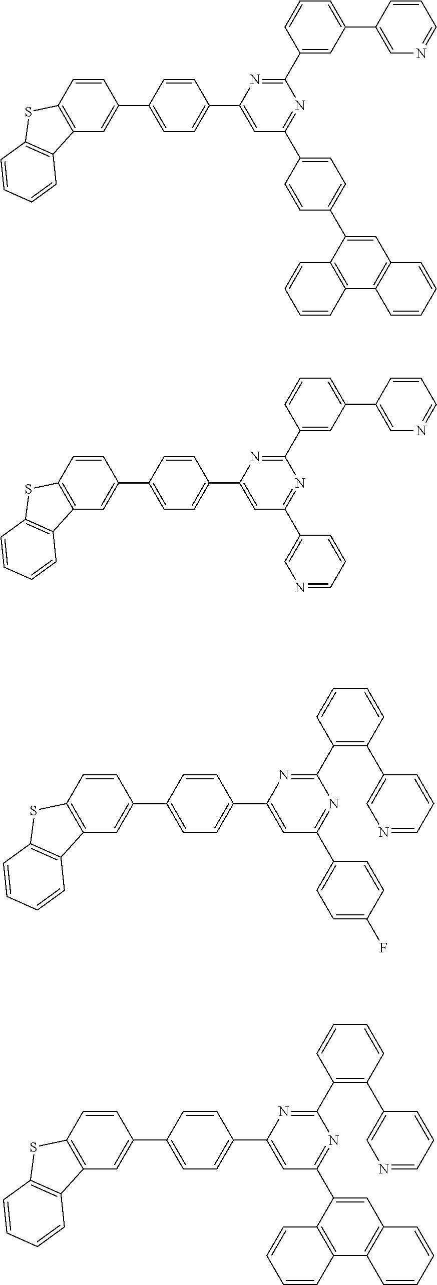 [SMILES: c1cncc(-c2cccc(-c3nc(-c4ccc(-c5ccc6sc7ccccc7c6c5)cc4)cc(-c4ccc(-c5cc6ccccc6c6ccccc56)cc4)n3)c2)c1 ; c1cncc(-c2cccc(-c3nc(-c4ccc(-c5ccc6sc7ccccc7c6c5)cc4)cc(-c4cccnc4)n3)c2)c1 ; Fc1ccc(-c2cc(-c3ccc(-c4ccc5sc6ccccc6c5c4)cc3)nc(-c3ccccc3-c3cccnc3)n2)cc1 ; c1cncc(-c2ccccc2-c2nc(-c3ccc(-c4ccc5sc6ccccc6c5c4)cc3)cc(-c3cc4ccccc4c4ccccc34)n2)c1]

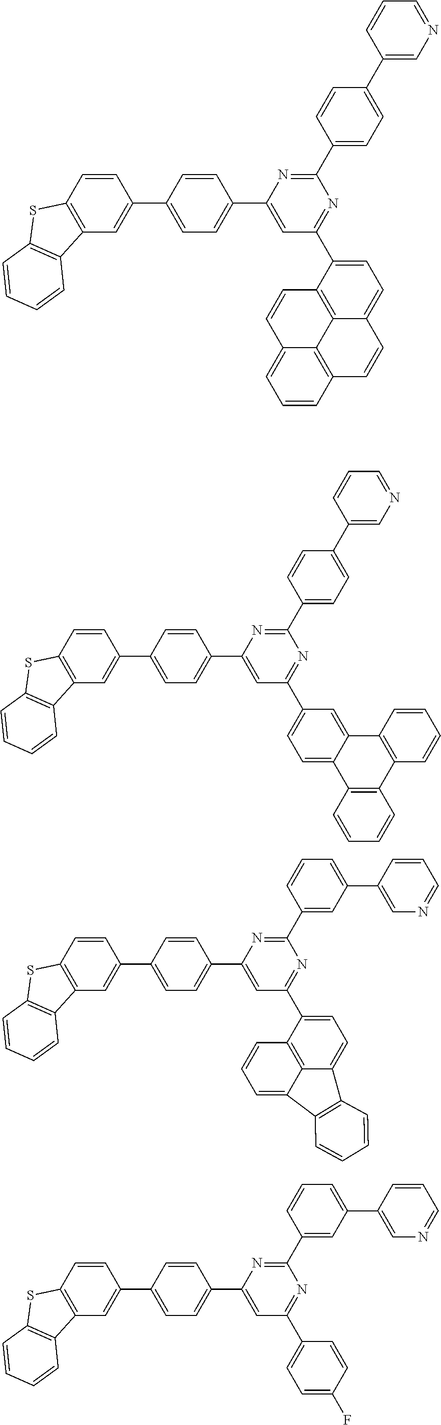

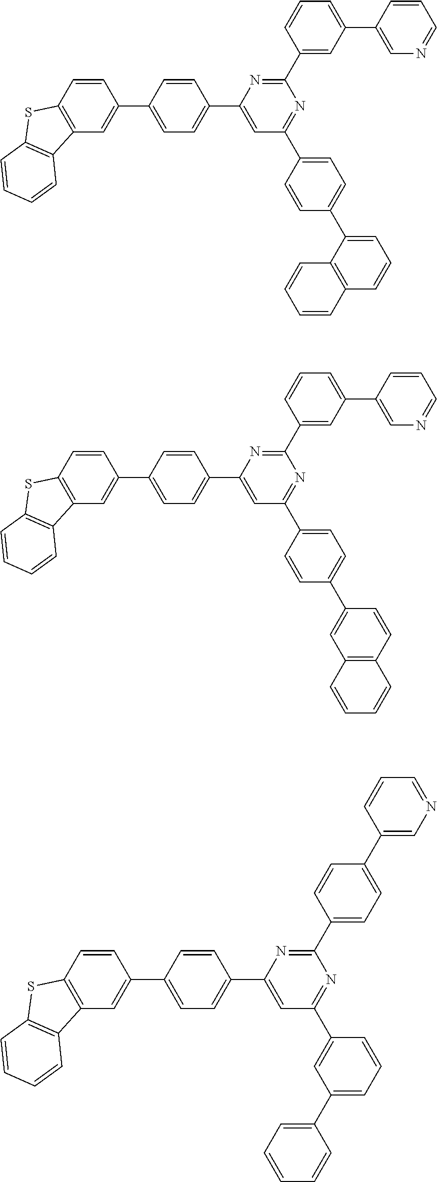 [SMILES: c1cncc(-c2cccc(-c3nc(-c4ccc(-c5ccc6sc7ccccc7c6c5)cc4)cc(-c4ccc(-c5cccc6ccccc56)cc4)n3)c2)c1 ; c1cncc(-c2cccc(-c3nc(-c4ccc(-c5ccc6ccccc6c5)cc4)cc(-c4ccc(-c5ccc6sc7ccccc7c6c5)cc4)n3)c2)c1 ; c1ccc(-c2cccc(-c3cc(-c4ccc(-c5ccc6sc7ccccc7c6c5)cc4)nc(-c4ccc(-c5cccnc5)cc4)n3)c2)cc1]

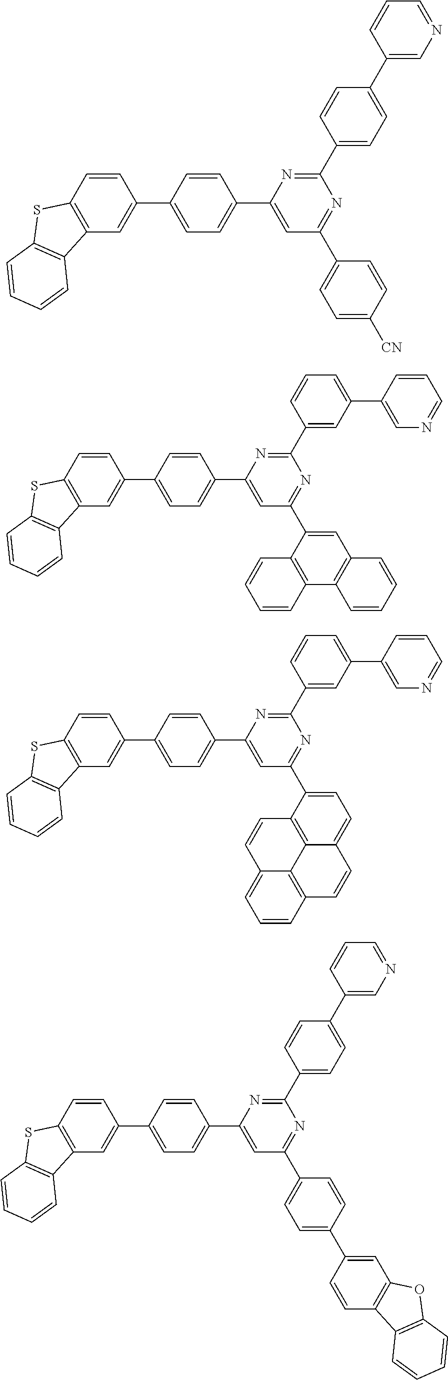

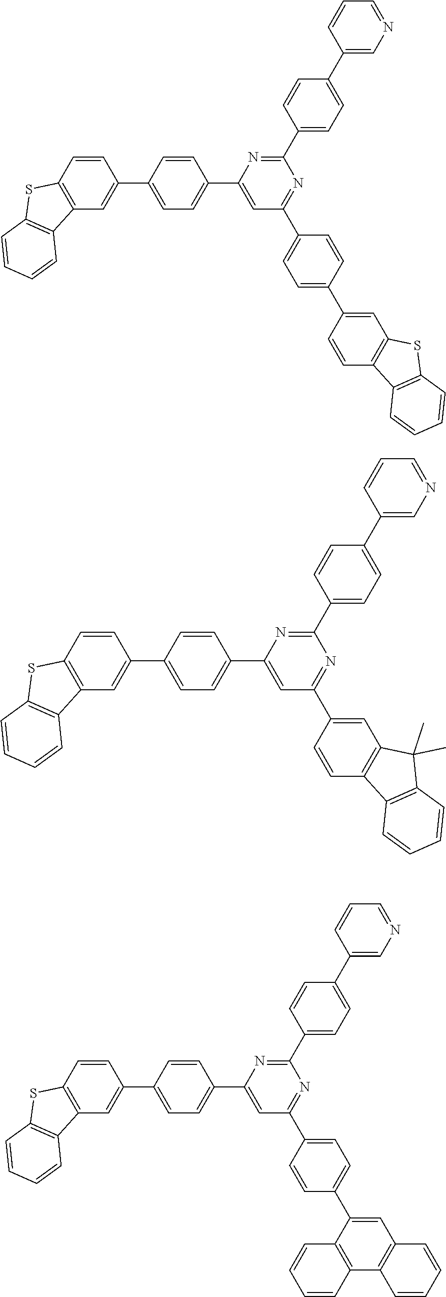

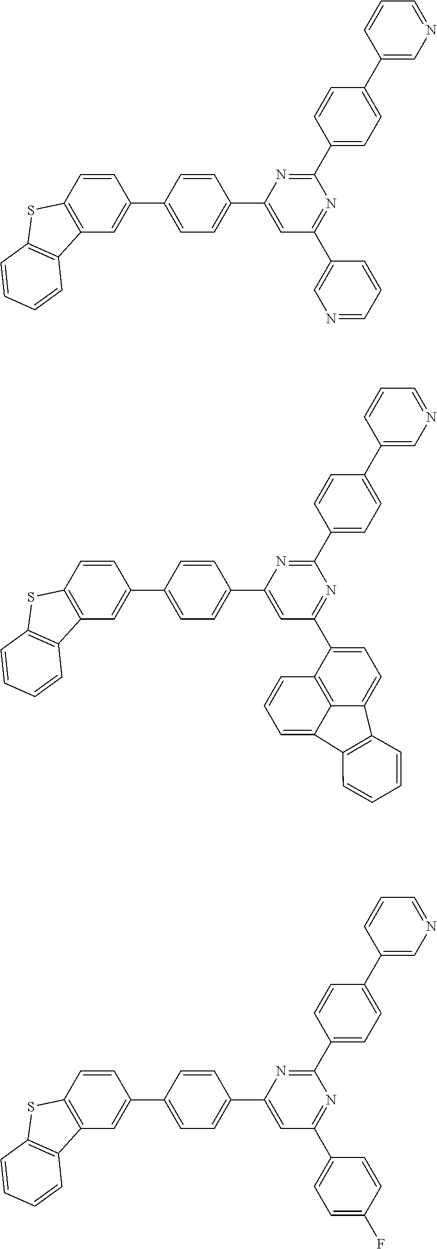 [SMILES: c1cncc(-c2ccc(-c3nc(-c4ccc(-c5ccc6sc7ccccc7c6c5)cc4)cc(-c4cccnc4)n3)cc2)c1 ; c1cncc(-c2ccc(-c3nc(-c4ccc(-c5ccc6sc7ccccc7c6c5)cc4)cc(-c4ccc5c6c(cccc46)-c4ccccc4-5)n3)cc2)c1 ; Fc1ccc(-c2cc(-c3ccc(-c4ccc5sc6ccccc6c5c4)cc3)nc(-c3ccc(-c4cccnc4)cc3)n2)cc1]

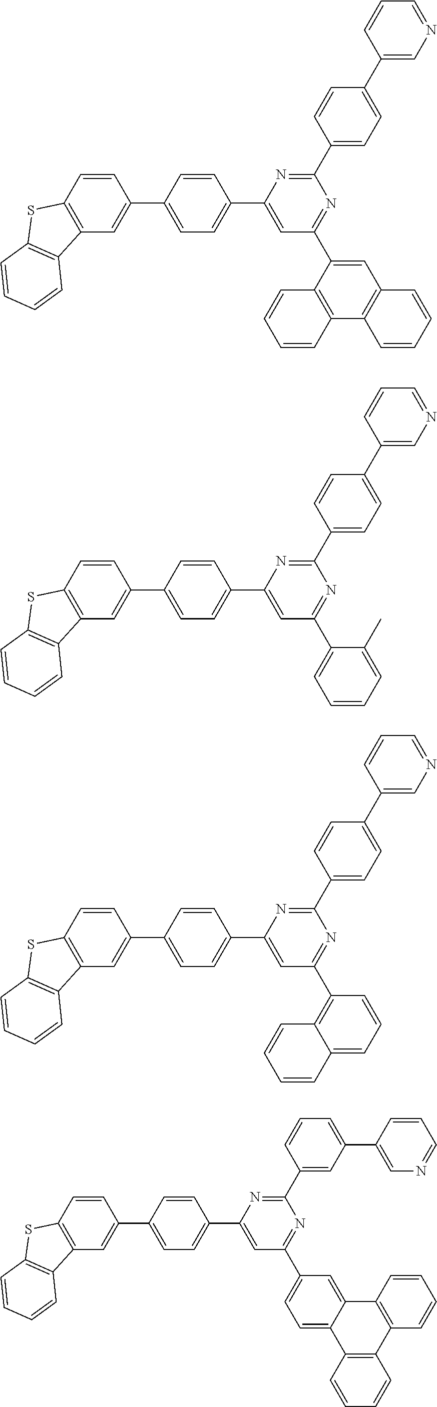

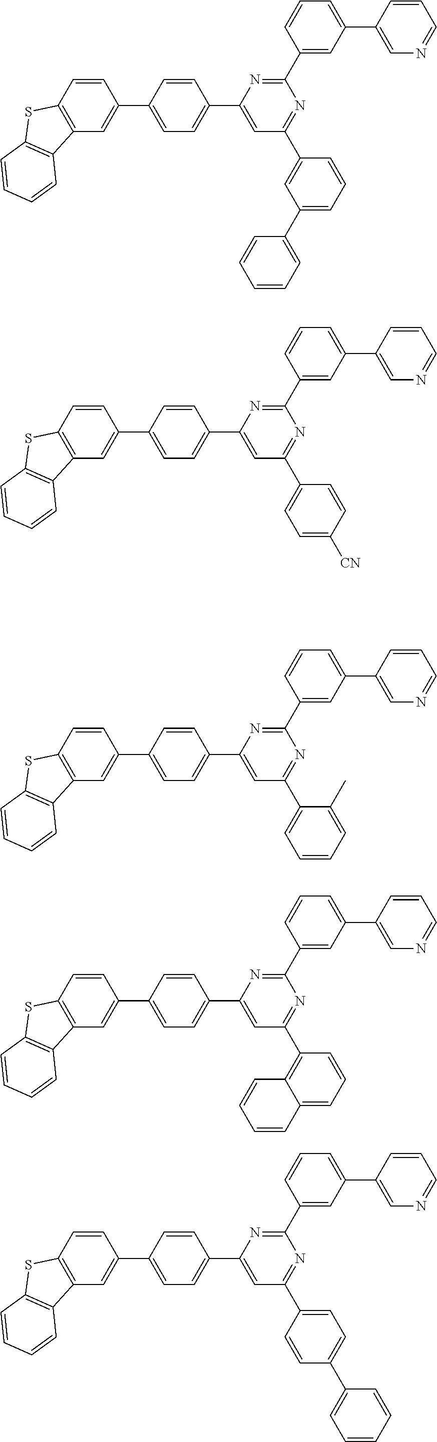

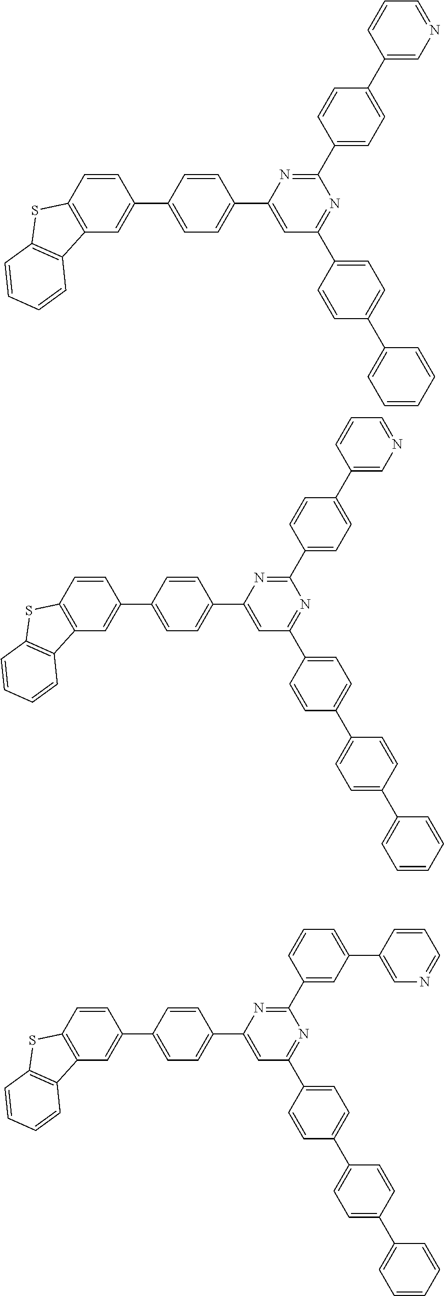

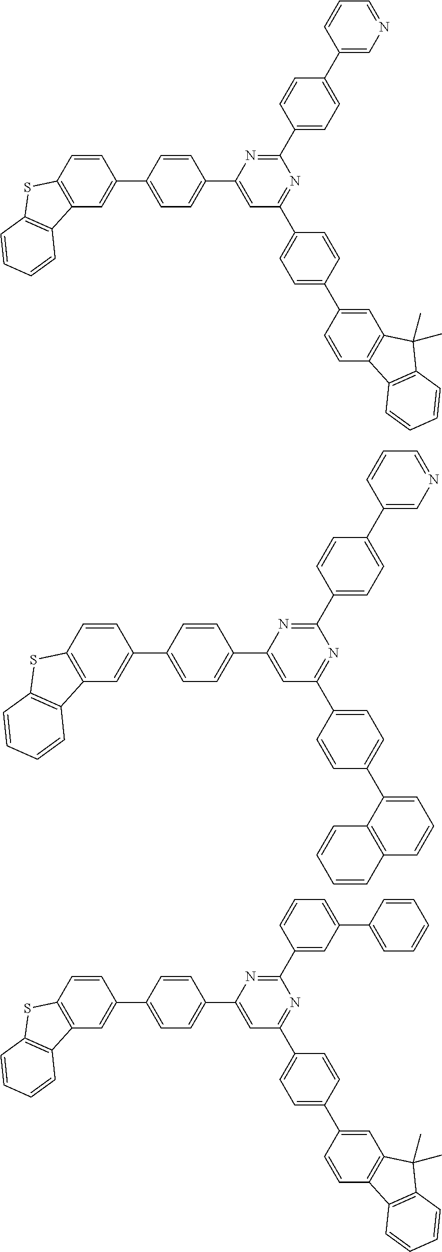

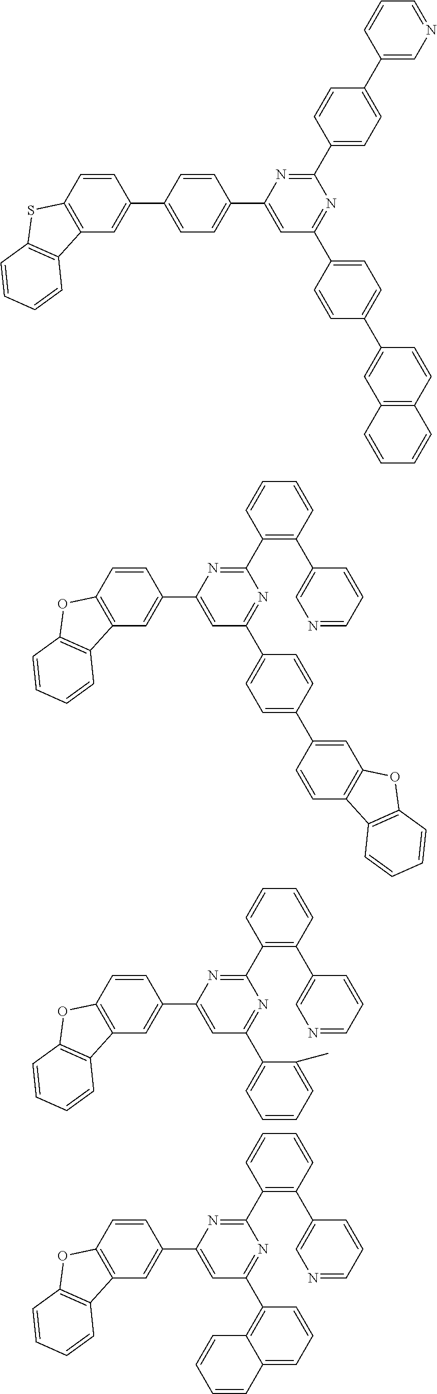

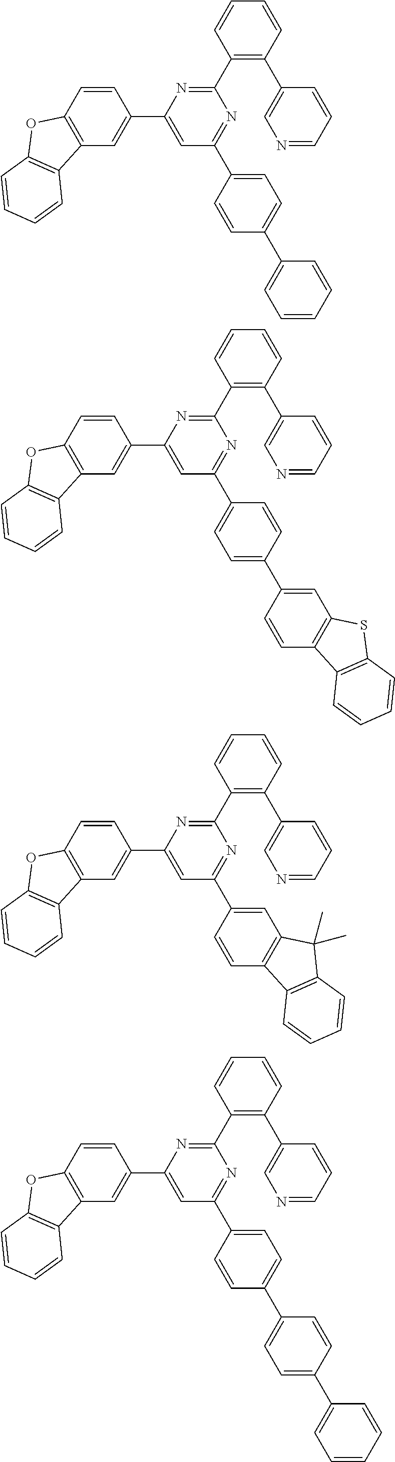

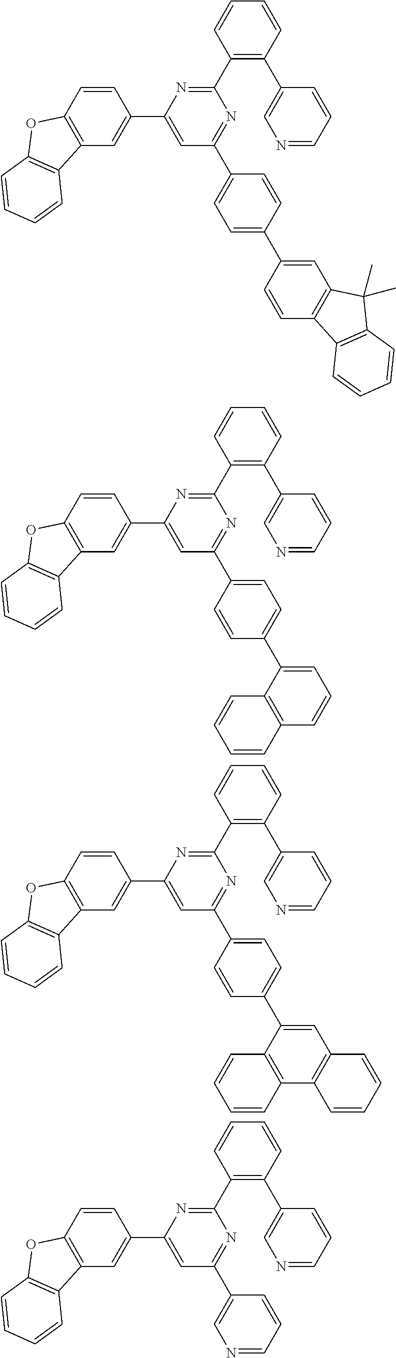

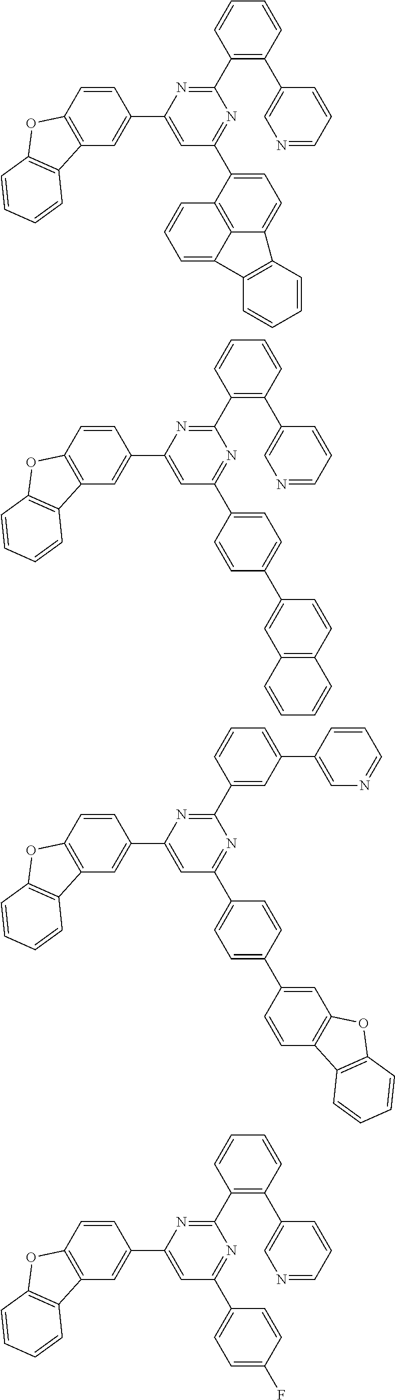

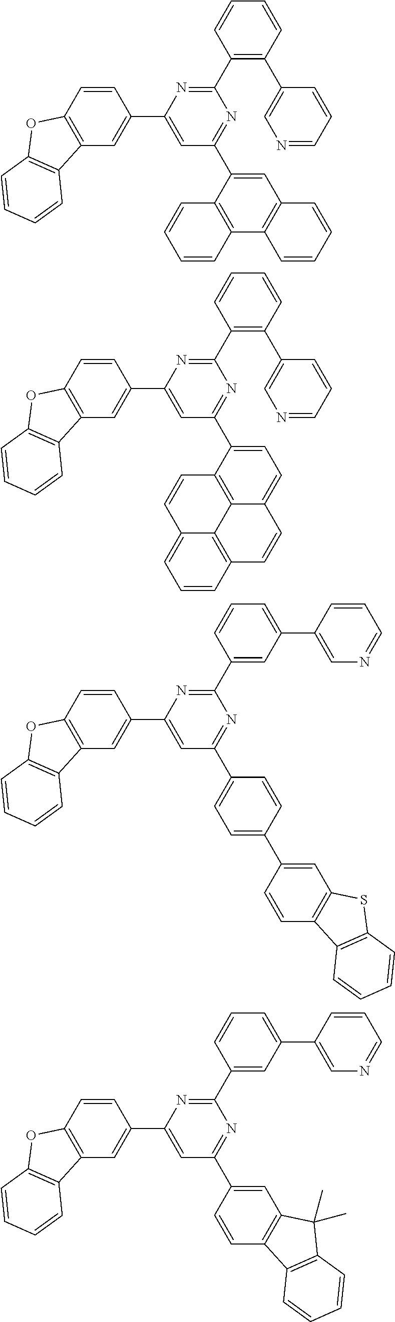

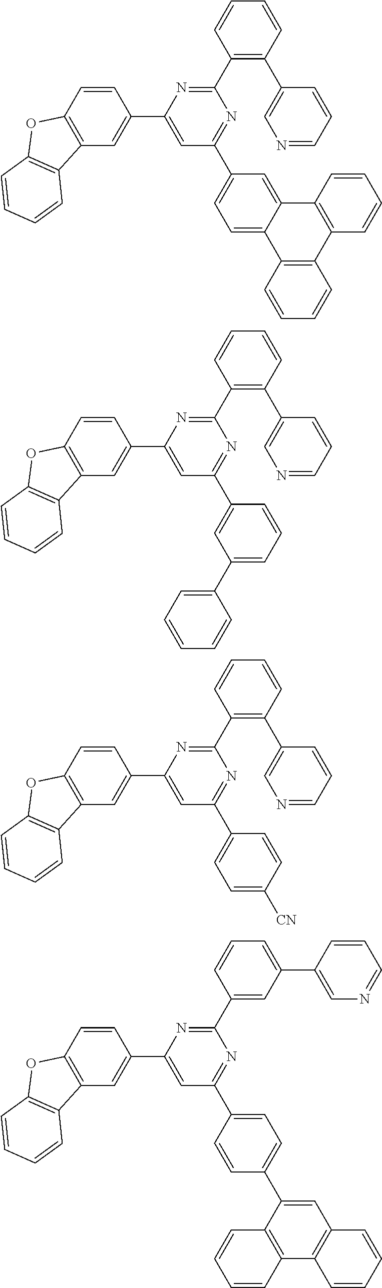

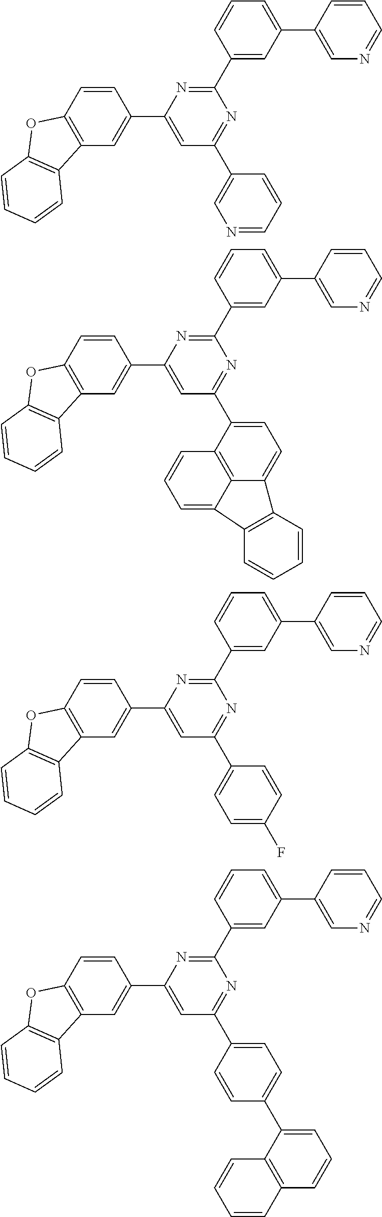

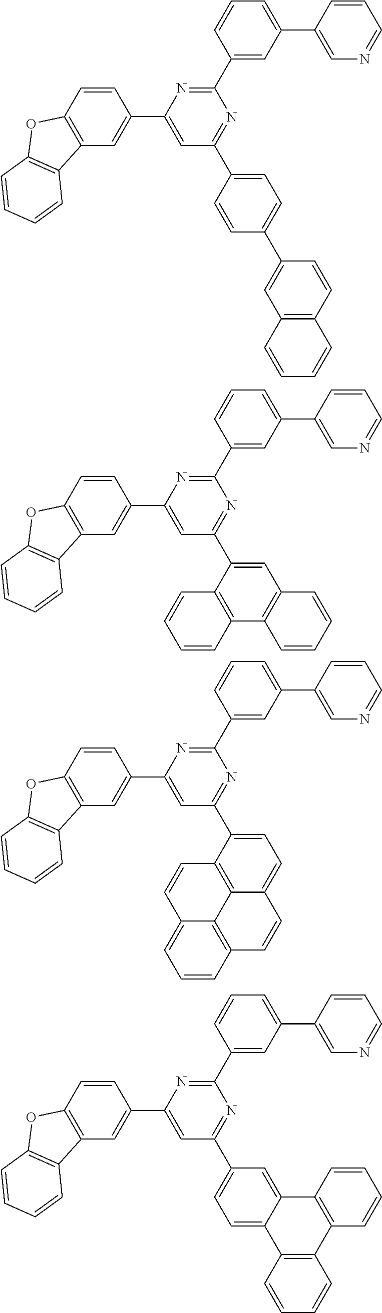

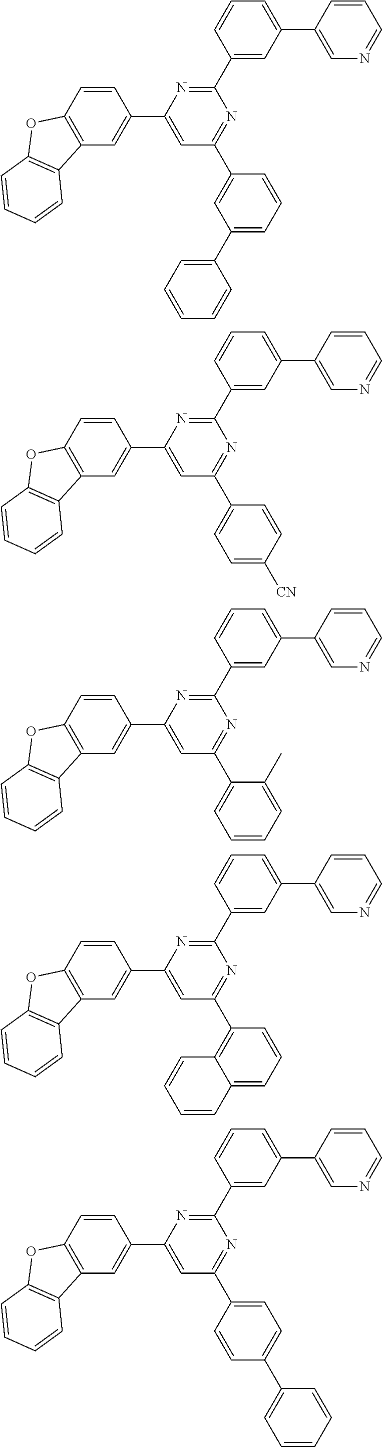

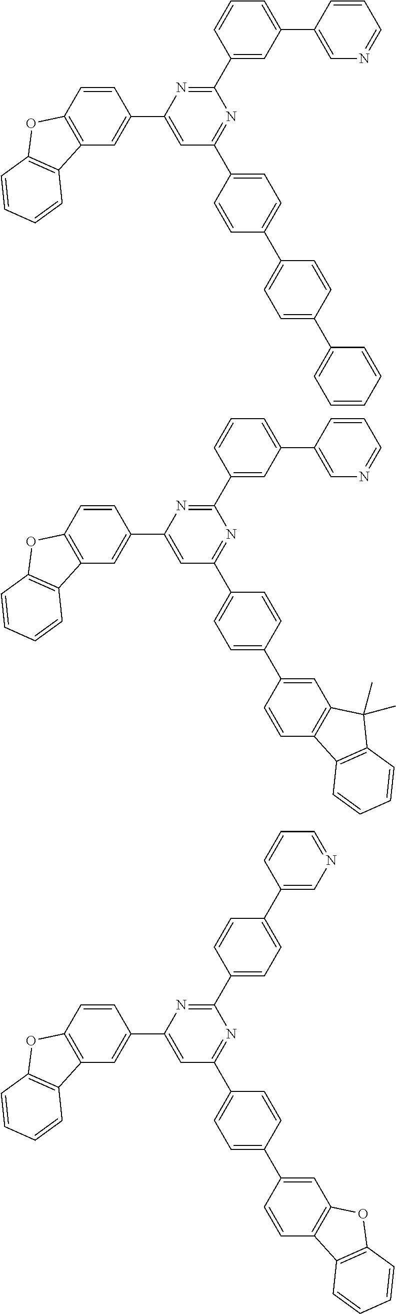

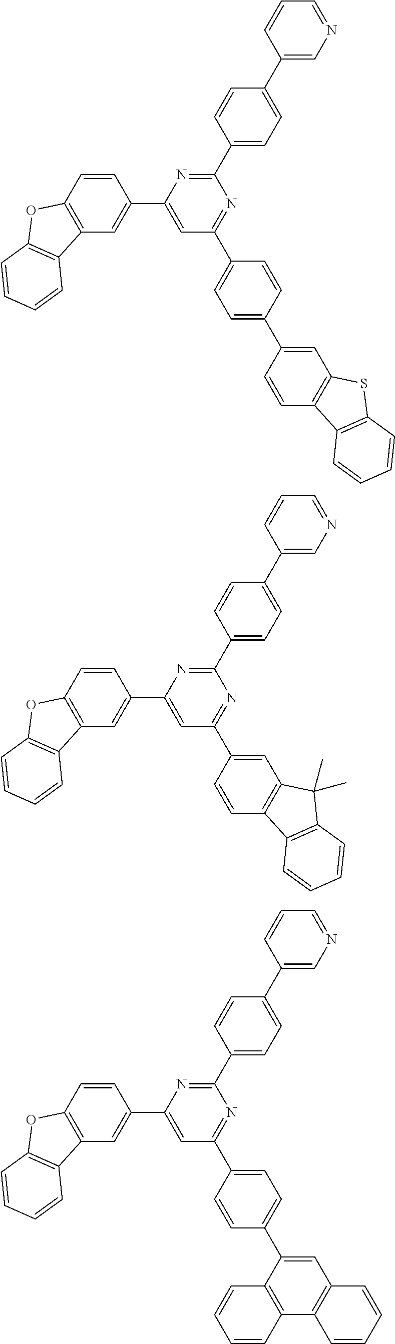

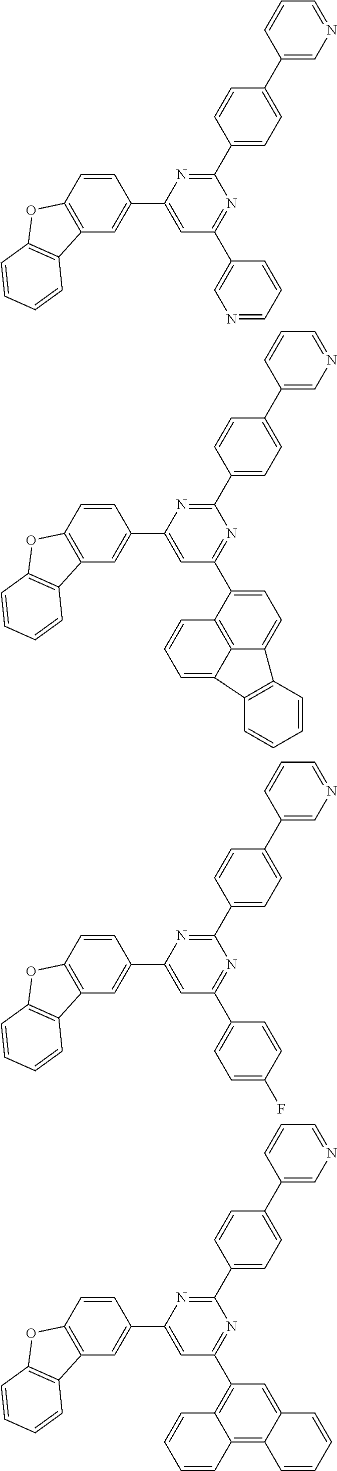

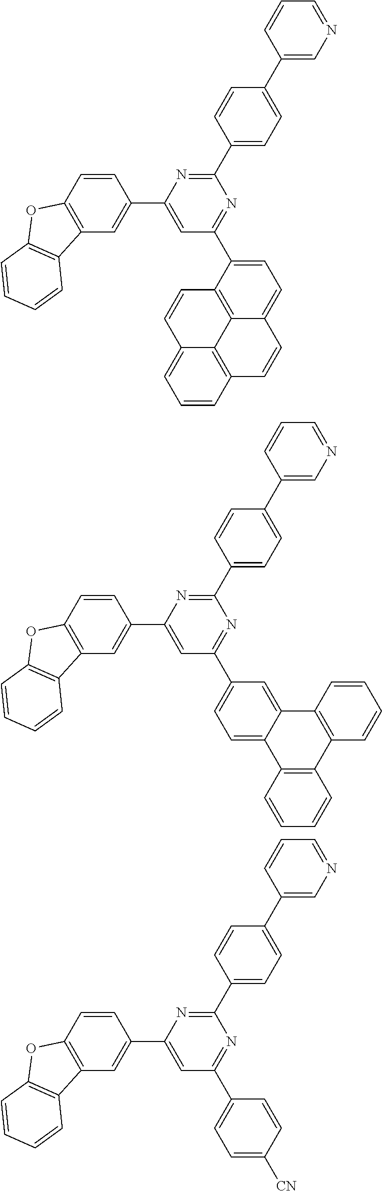

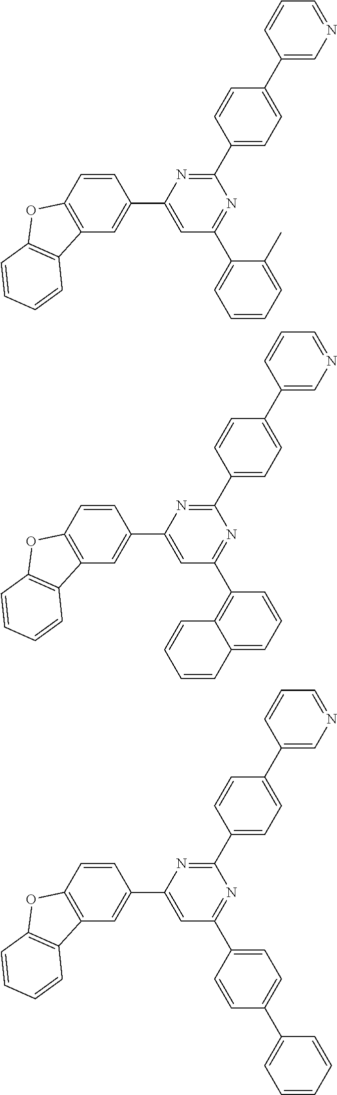

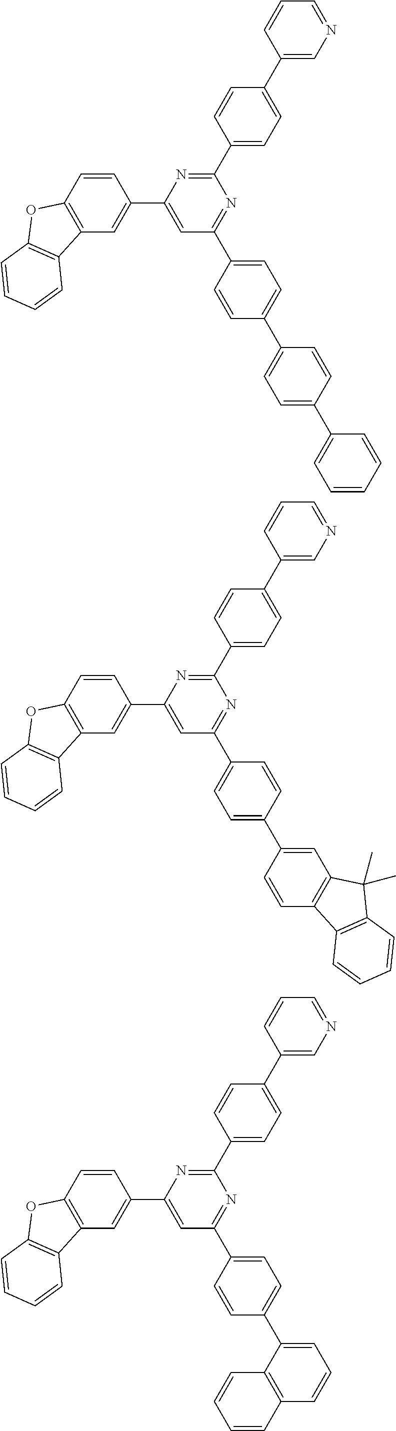

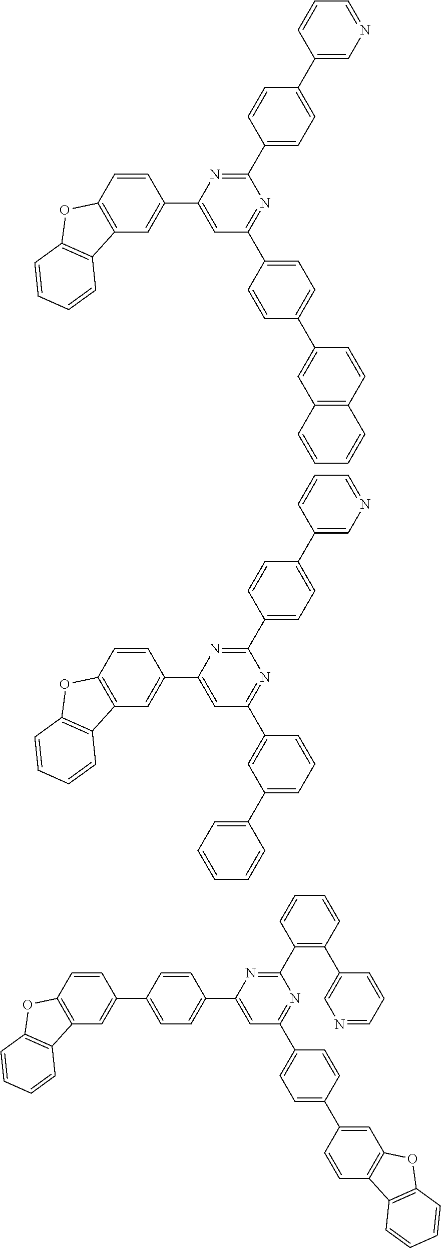

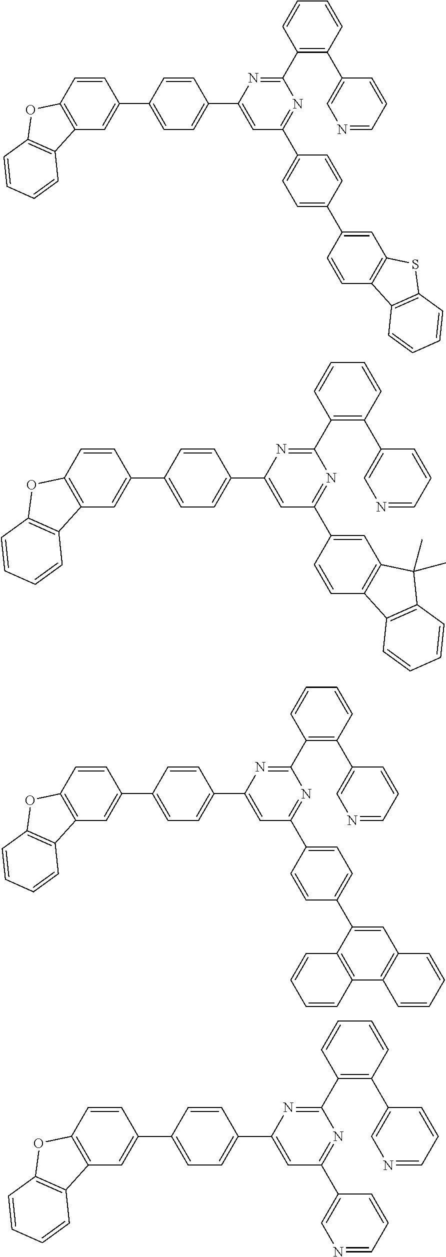

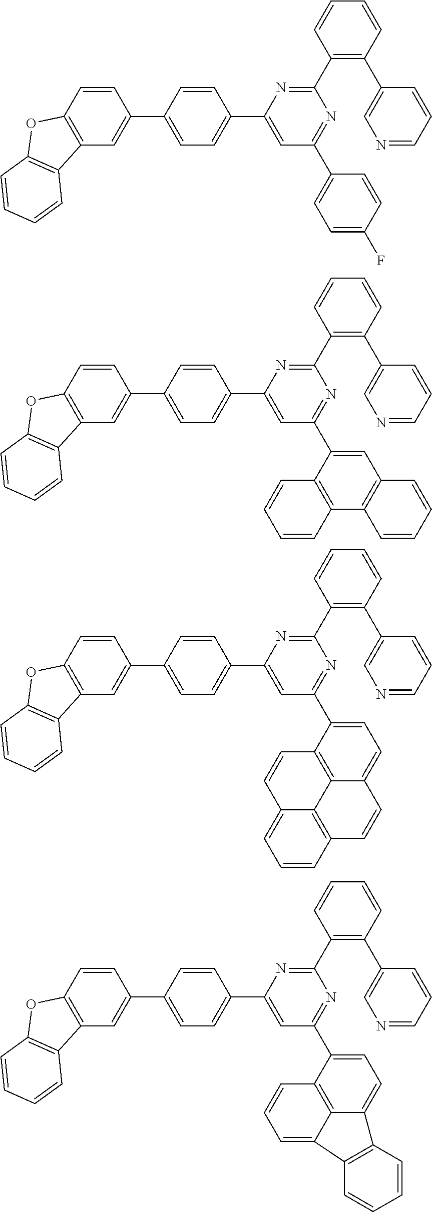

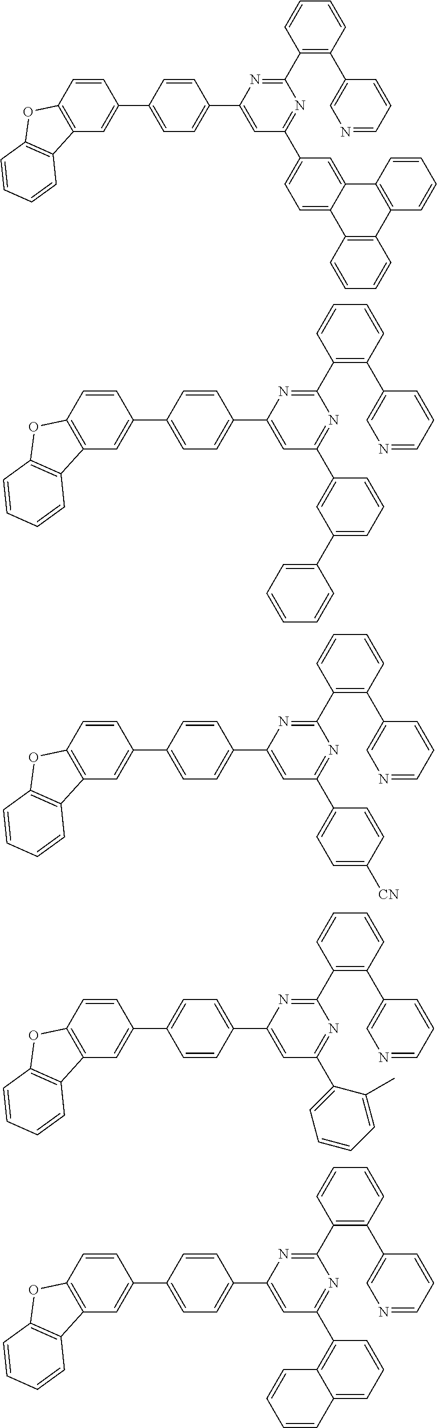

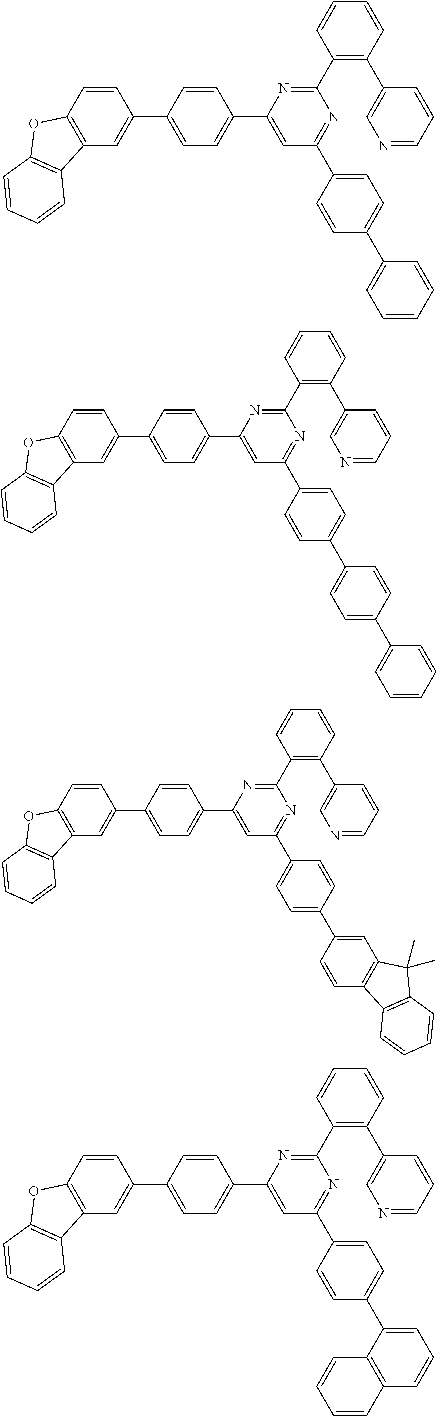

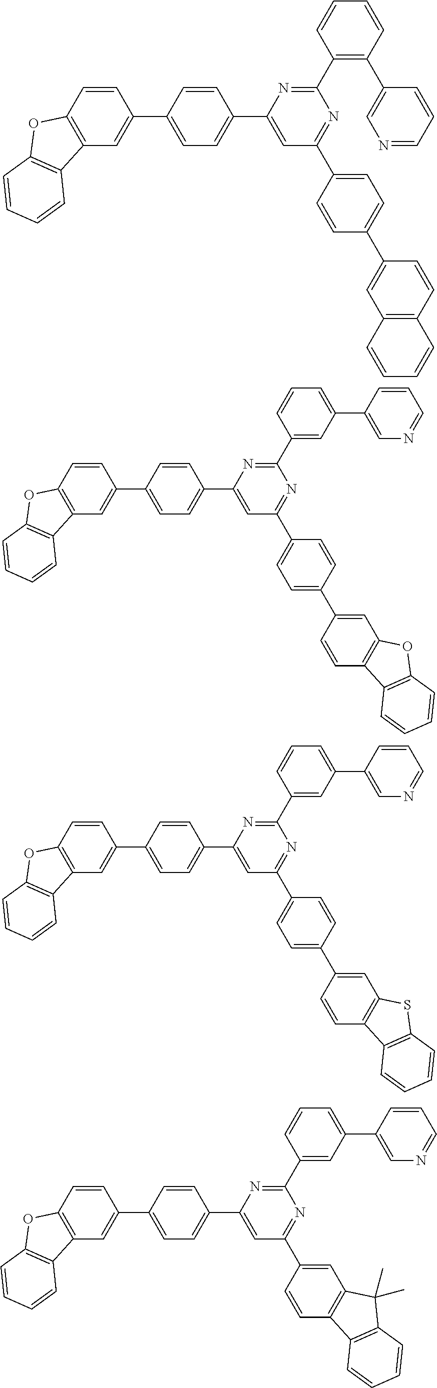

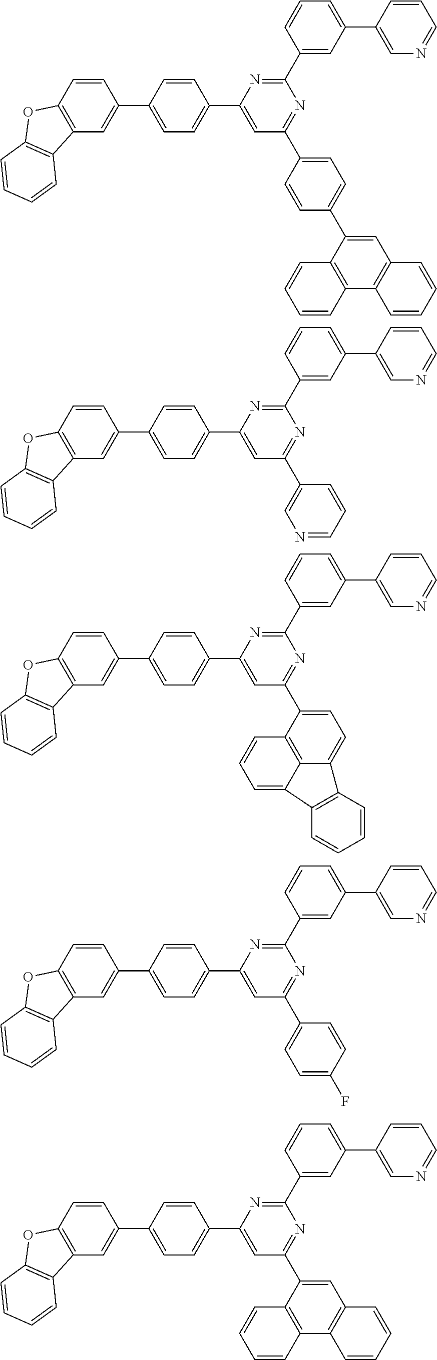

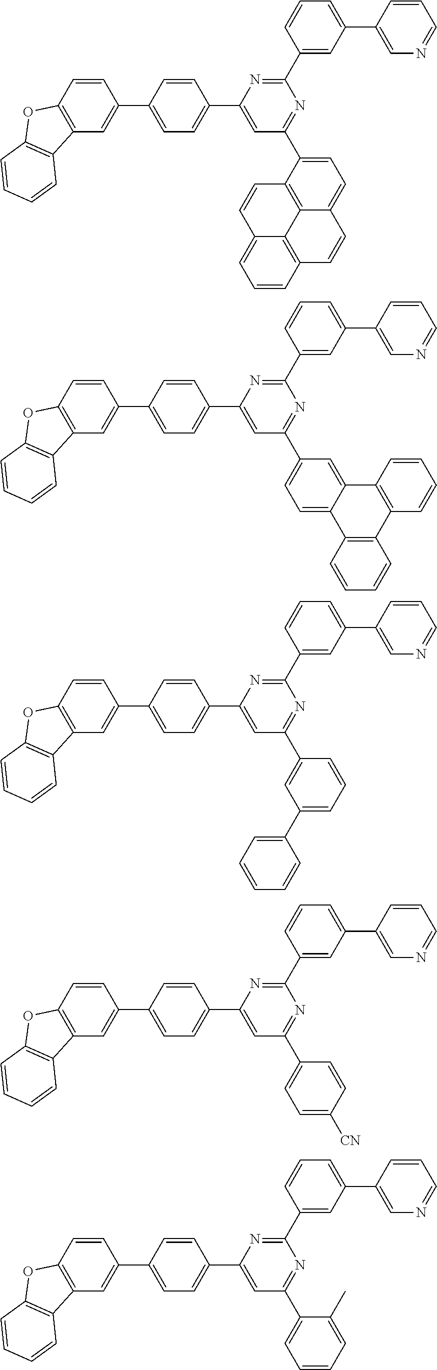

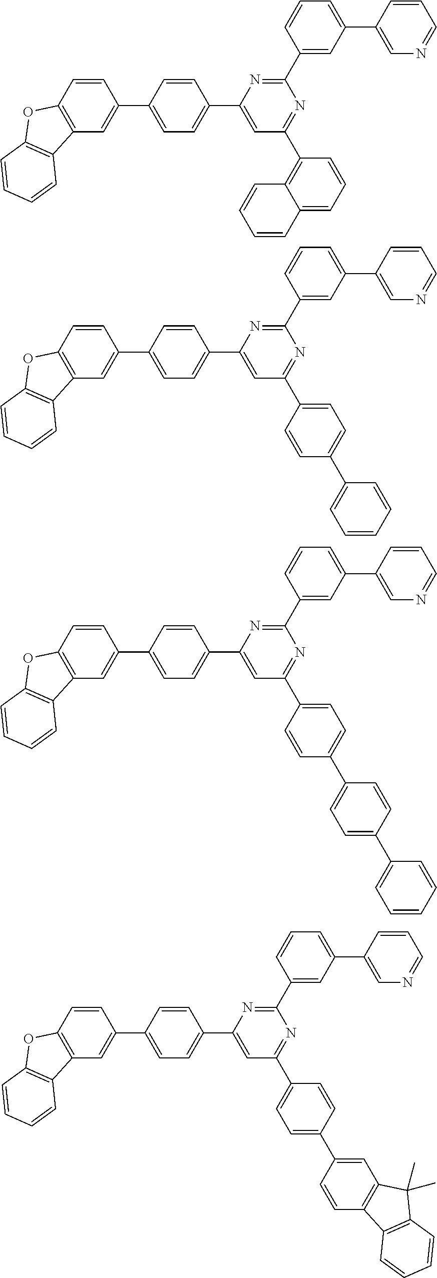

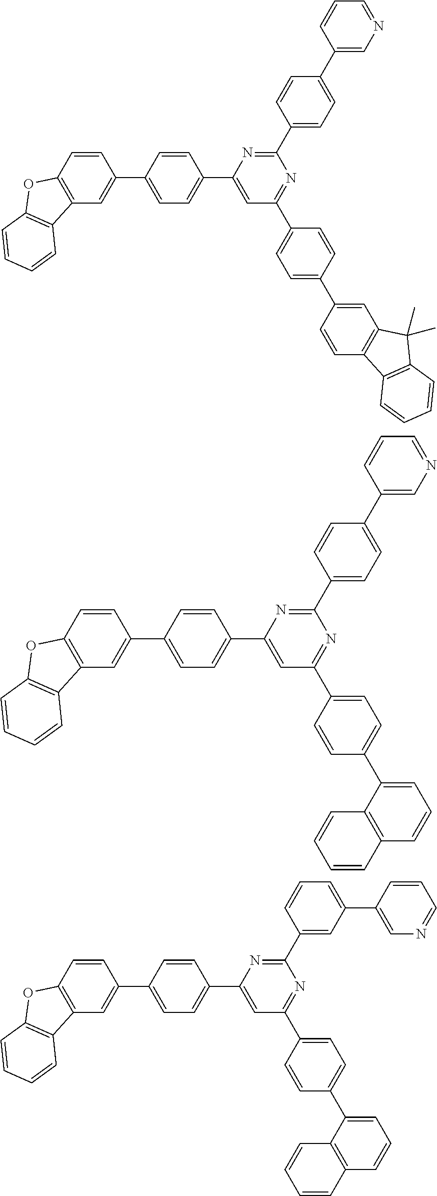

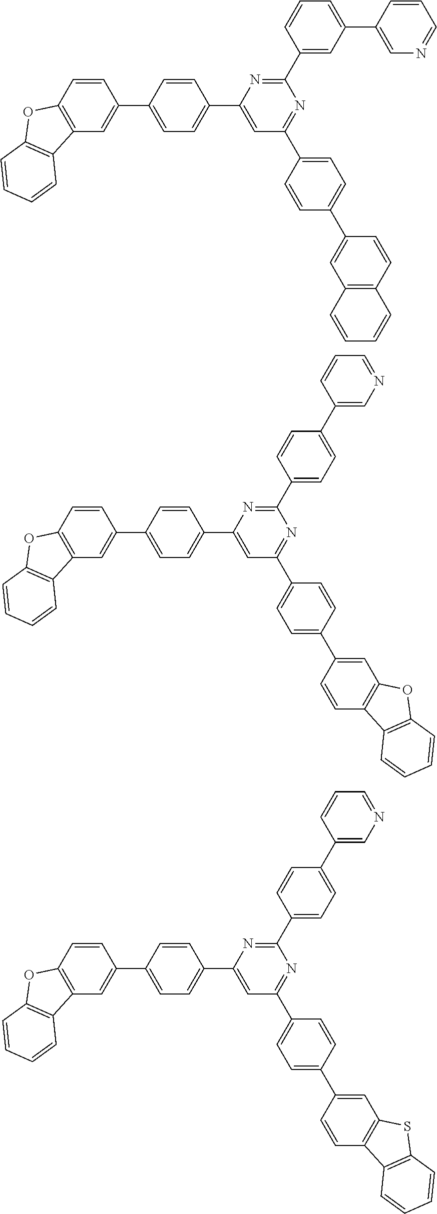

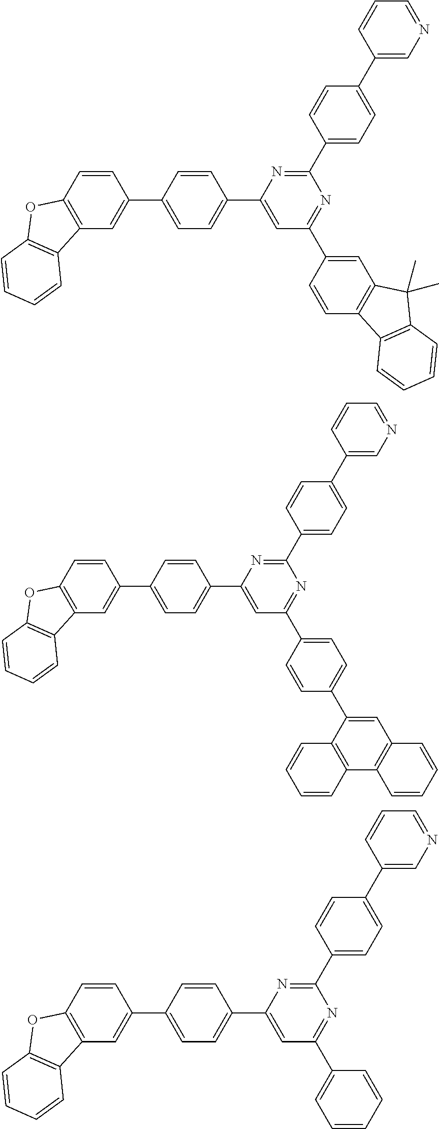

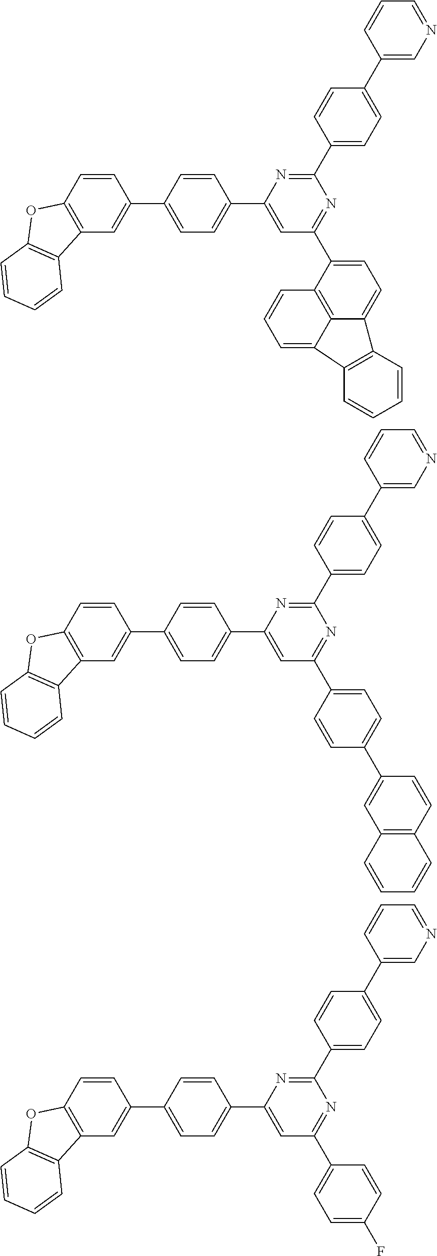

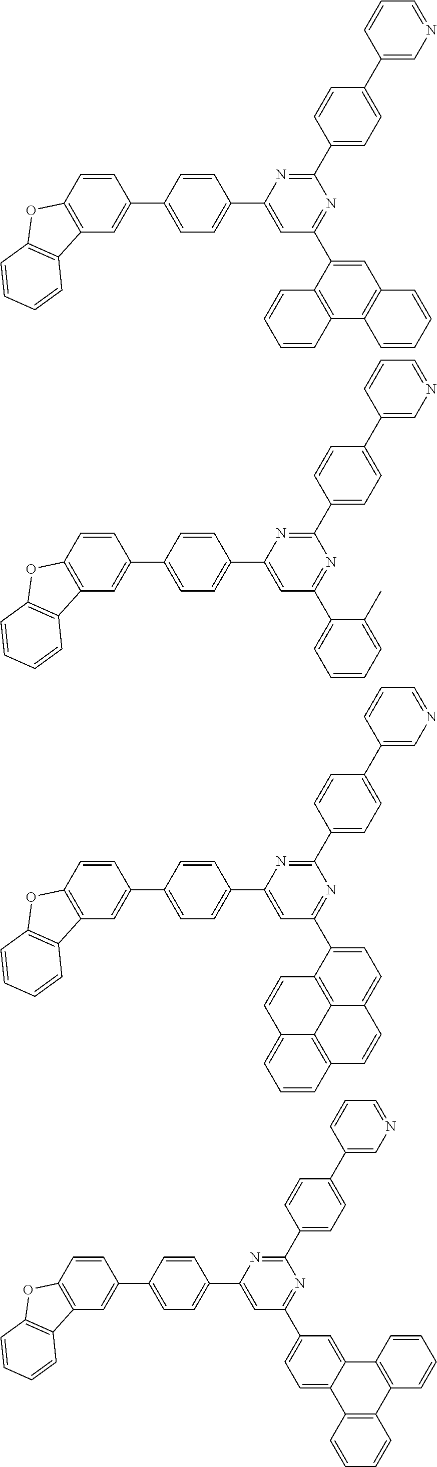

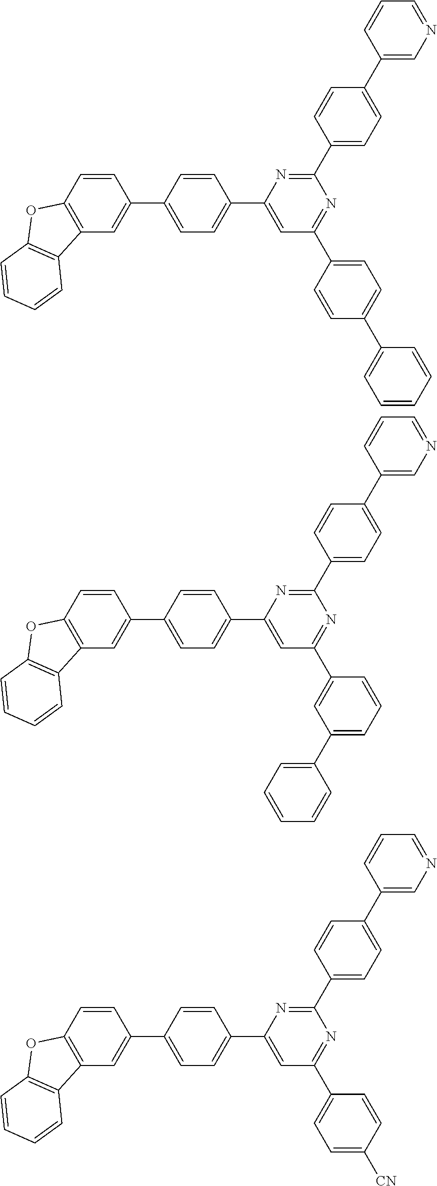

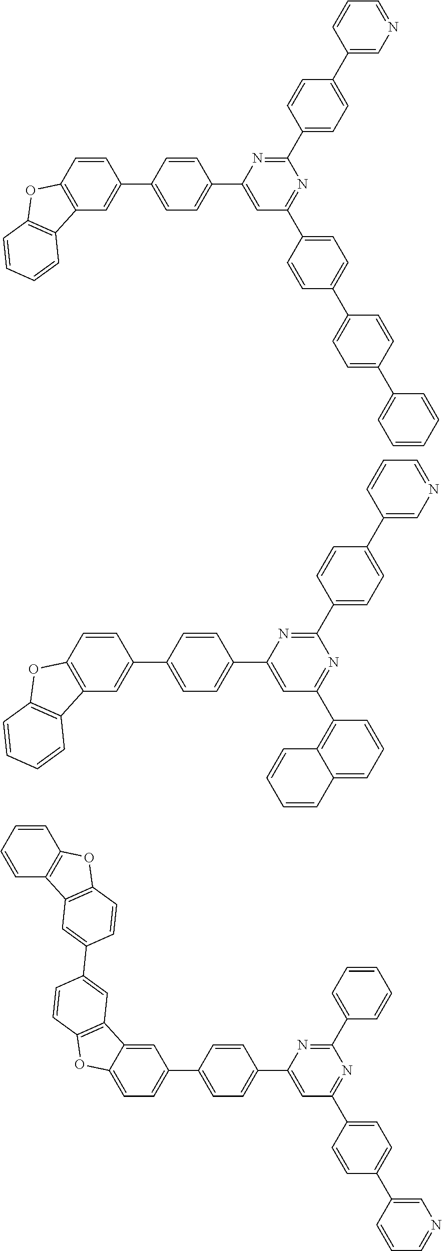

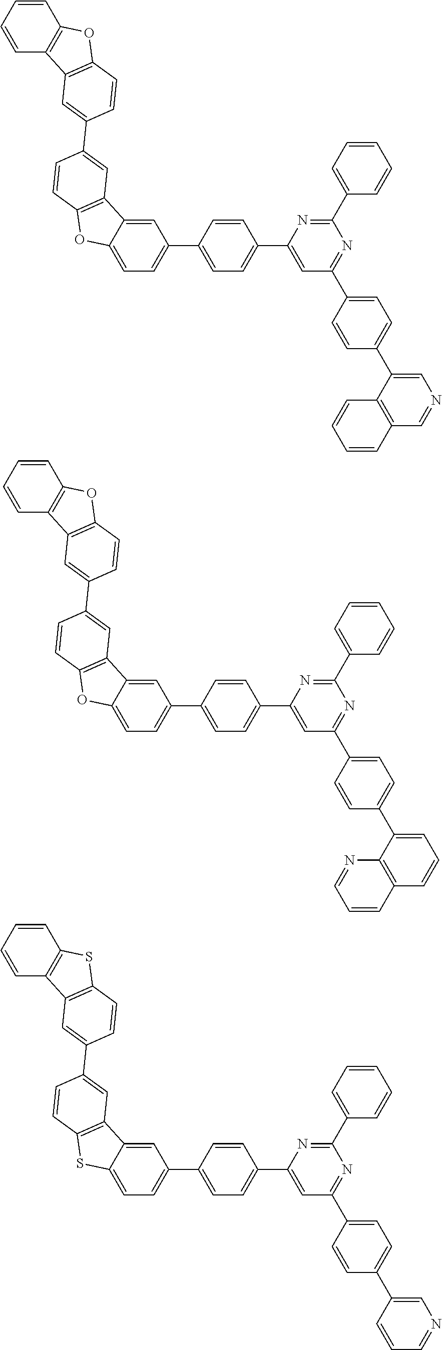 [SMILES: c1ccc(-c2nc(-c3ccc(-c4ccc5oc6ccc(-c7ccc8oc9ccccc9c8c7)cc6c5c4)cc3)cc(-c3ccc(-c4cncc5ccccc45)cc3)n2)cc1 ; c1ccc(-c2nc(-c3ccc(-c4ccc5oc6ccc(-c7ccc8oc9ccccc9c8c7)cc6c5c4)cc3)cc(-c3ccc(-c4cccc5cccnc45)cc3)n2)cc1 ; c1ccc(-c2nc(-c3ccc(-c4cccnc4)cc3)cc(-c3ccc(-c4ccc5sc6ccc(-c7ccc8sc9ccccc9c8c7)cc6c5c4)cc3)n2)cc1]

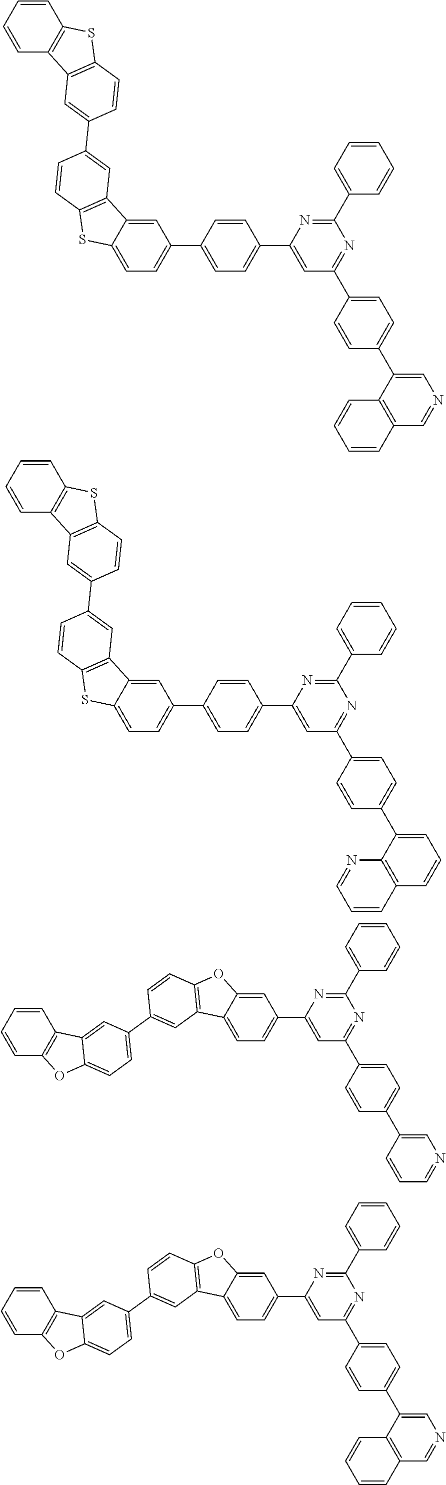

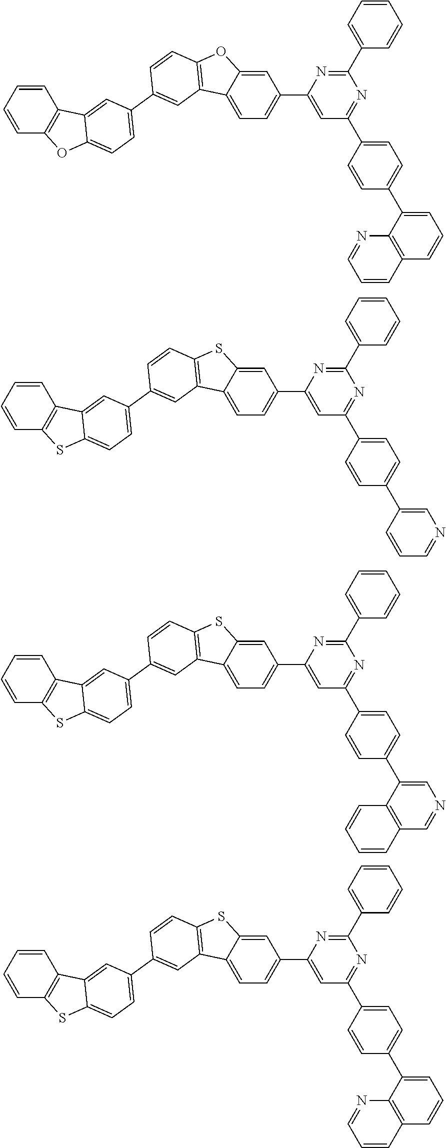

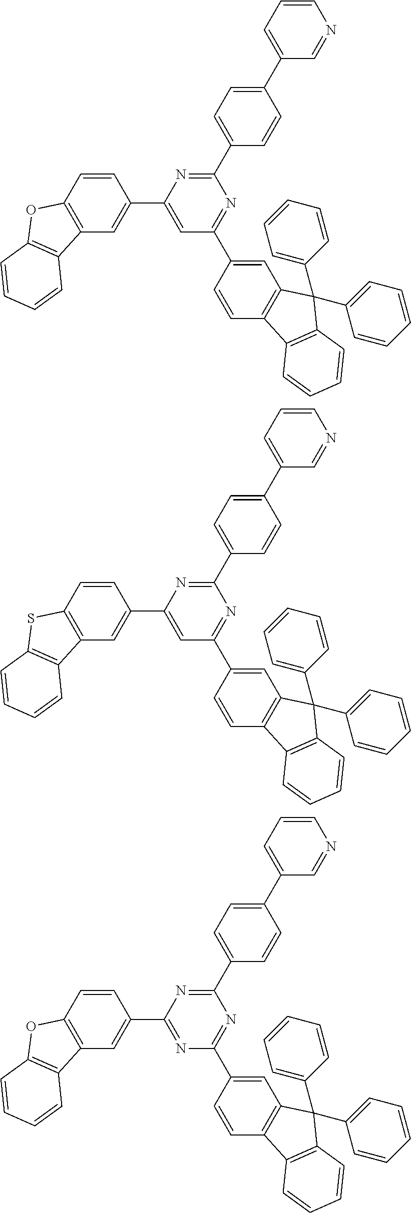

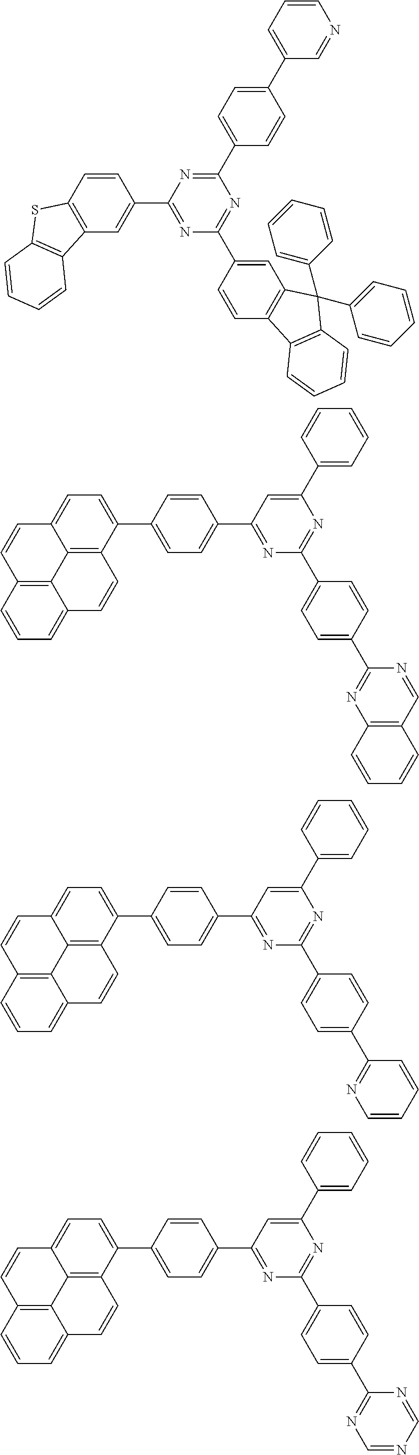

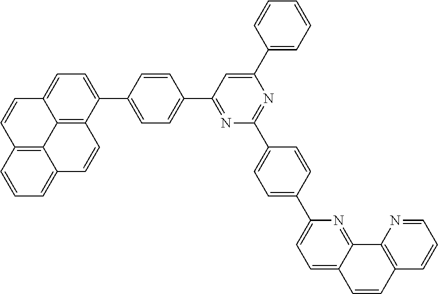 [SMILES: c1ccc(-c2cc(-c3ccc(-c4ccc5ccc6cccc7ccc4c5c67)cc3)nc(-c3ccc(-c4ccc5ccc6cccnc6c5n4)cc3)n2)cc1]

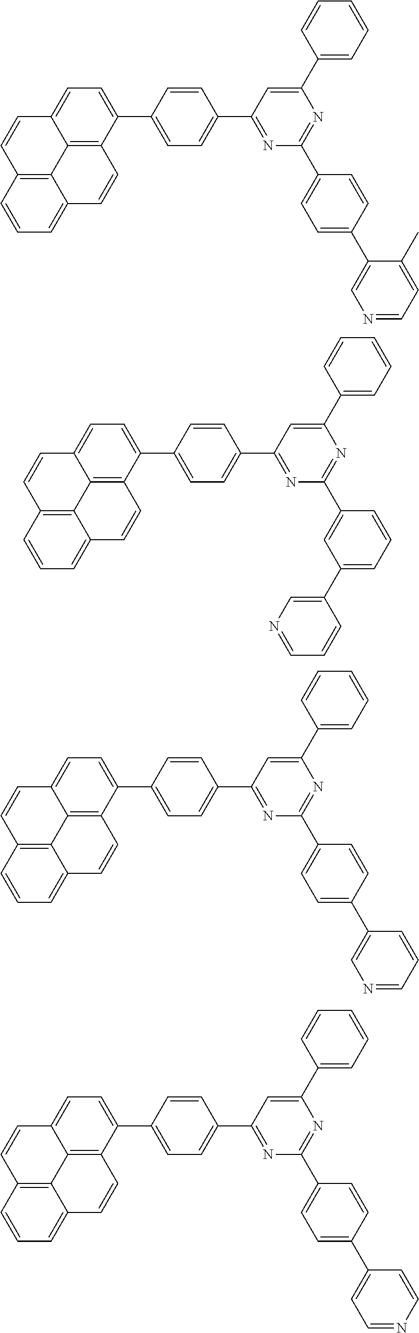

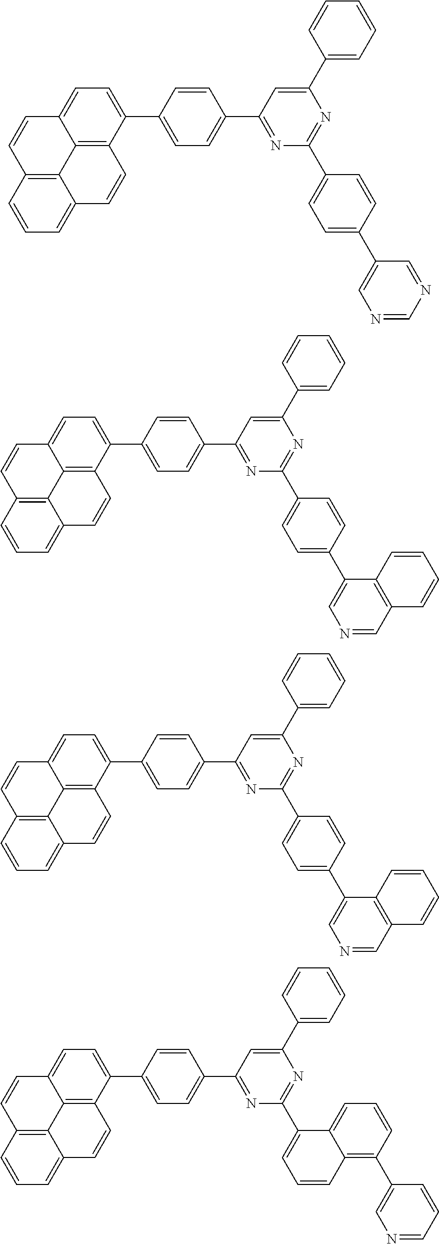

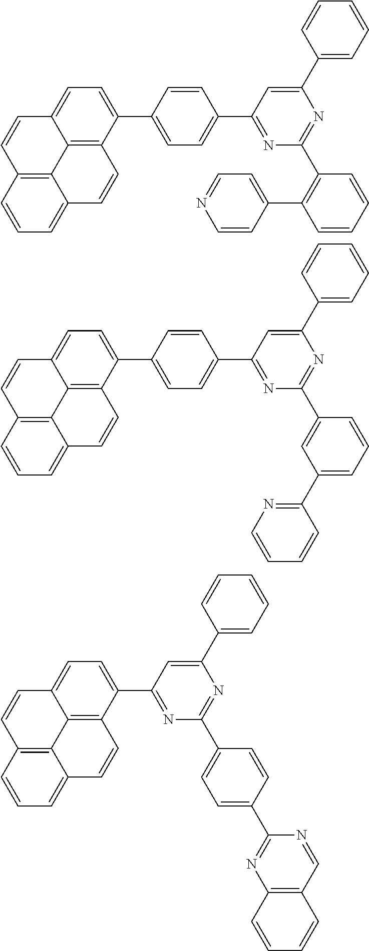

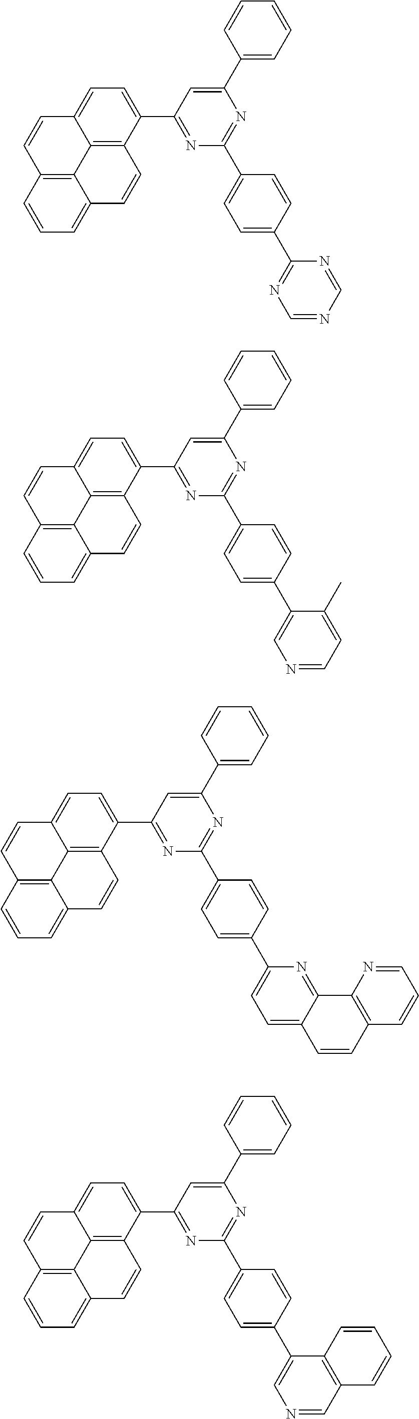

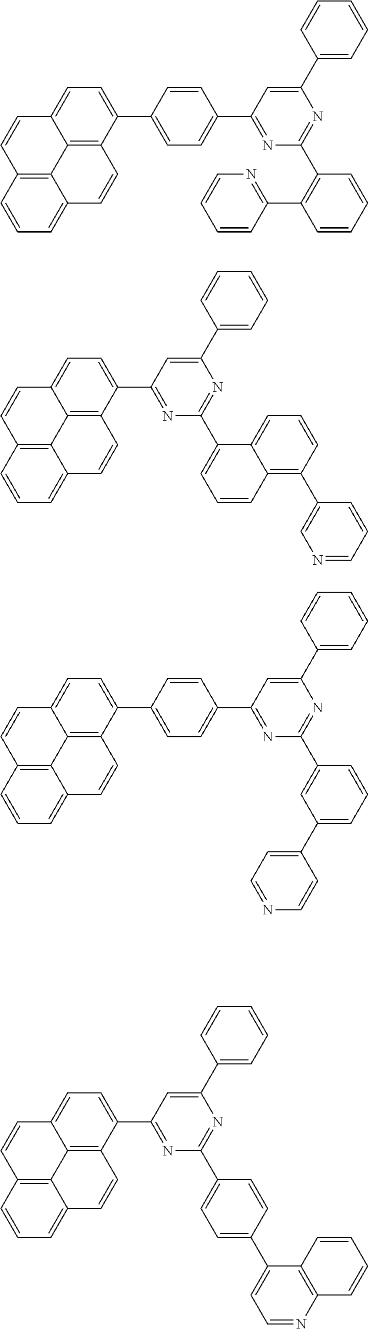

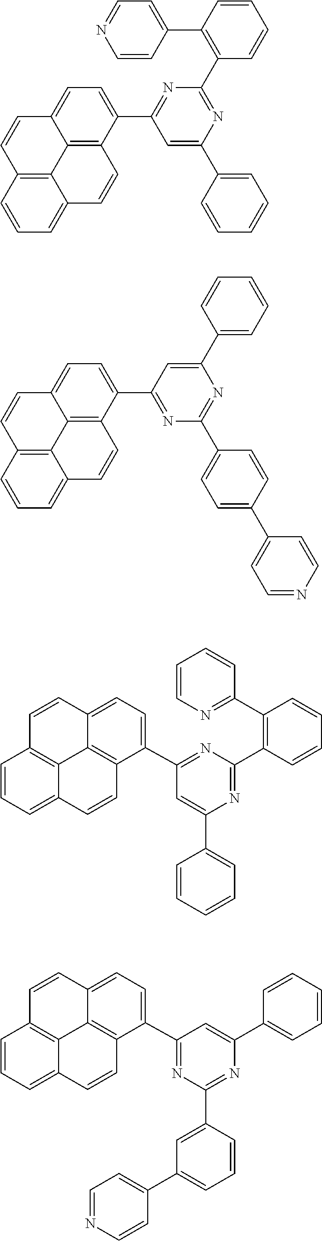

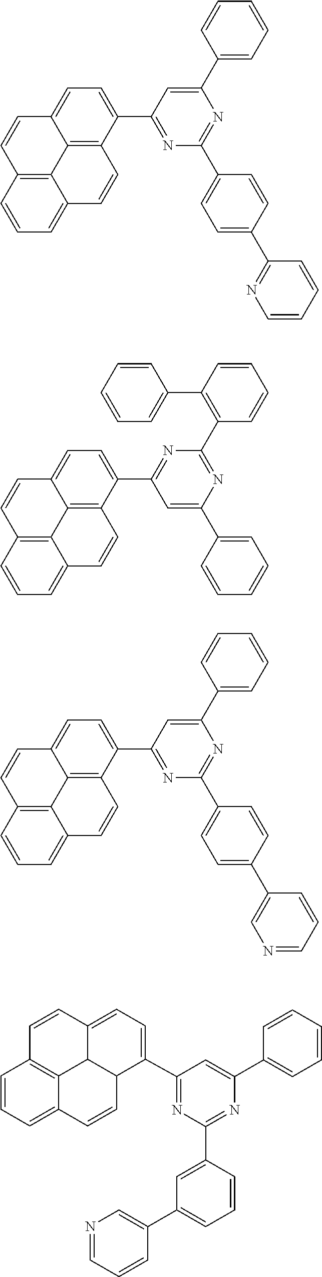

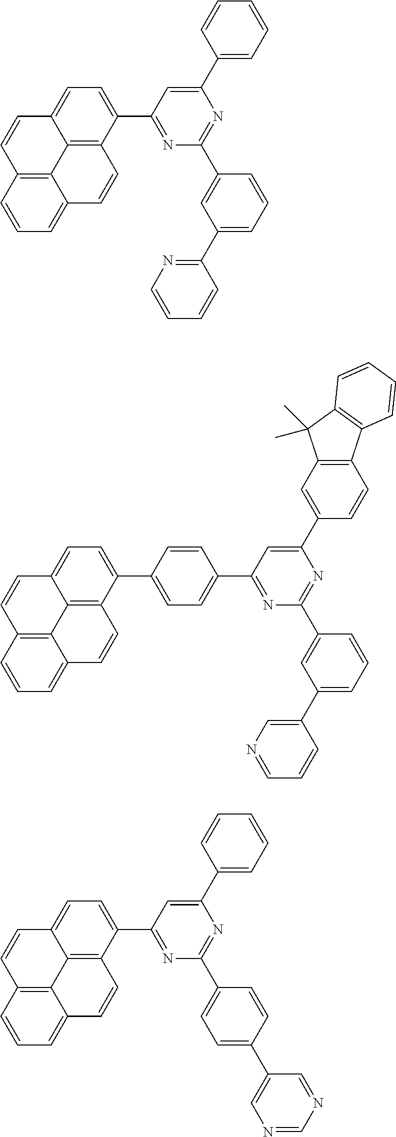

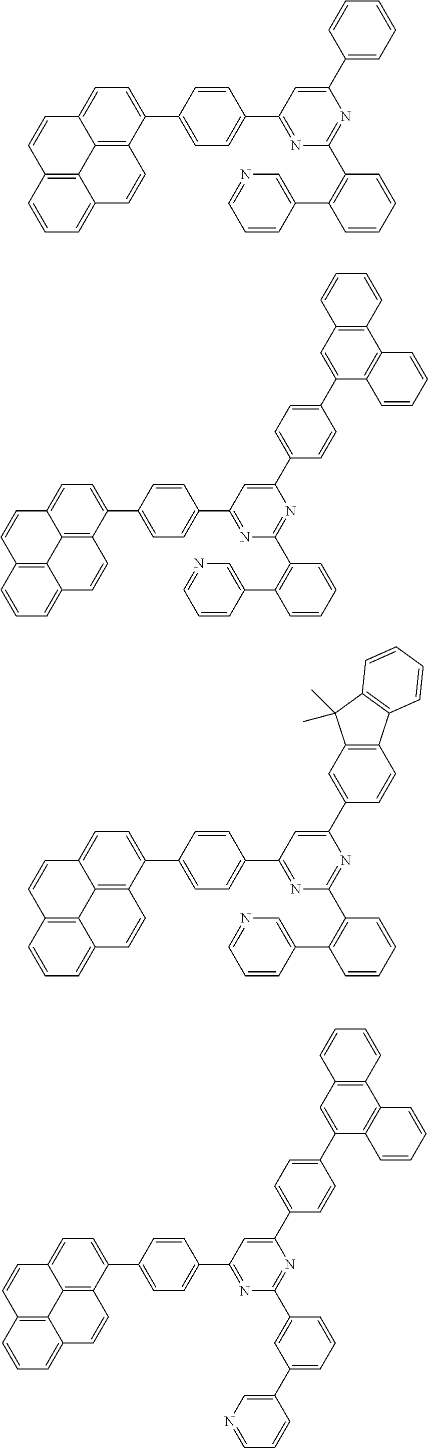 [SMILES: c1ccc(-c2cc(-c3ccc(-c4ccc5ccc6cccc7ccc4c5c67)cc3)nc(-c3ccccc3-c3cccnc3)n2)cc1 ; c1cncc(-c2ccccc2-c2nc(-c3ccc(-c4cc5ccccc5c5ccccc45)cc3)cc(-c3ccc(-c4ccc5ccc6cccc7ccc4c5c67)cc3)n2)c1 ; CC1(C)c2ccccc2-c2ccc(-c3cc(-c4ccc(-c5ccc6ccc7cccc8ccc5c6c78)cc4)nc(-c4ccccc4-c4cccnc4)n3)cc21 ; c1cncc(-c2cccc(-c3nc(-c4ccc(-c5cc6ccccc6c6ccccc56)cc4)cc(-c4ccc(-c5ccc6ccc7cccc8ccc5c6c78)cc4)n3)c2)c1]

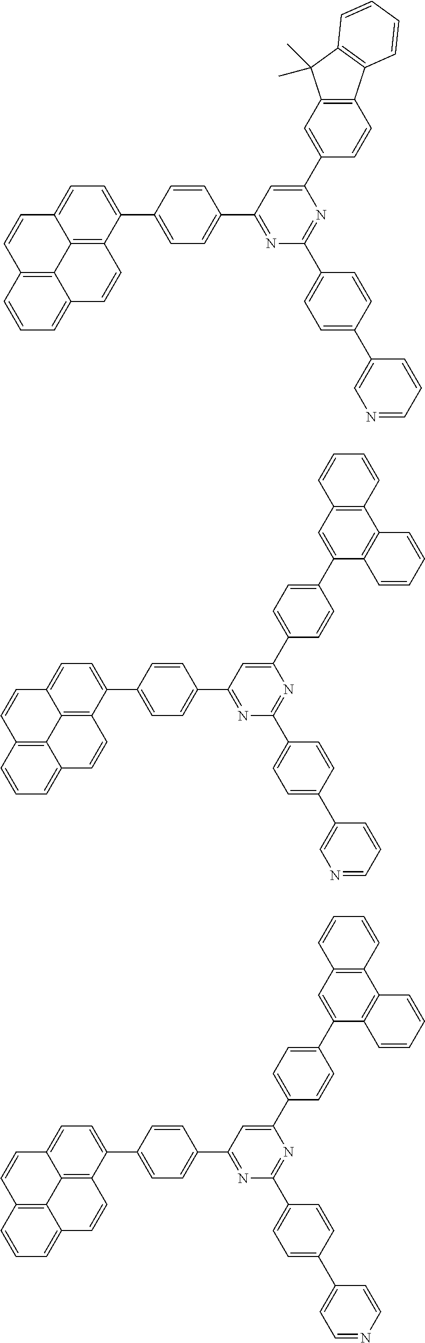

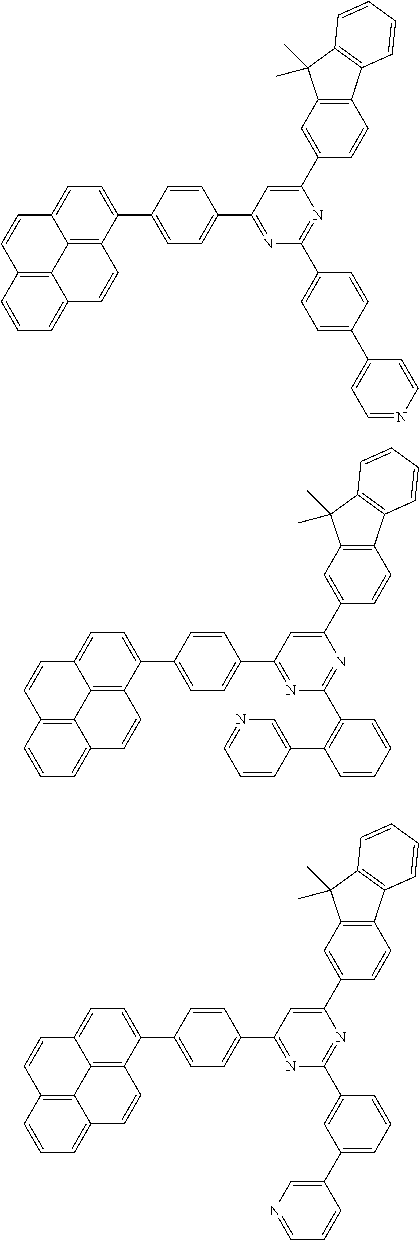

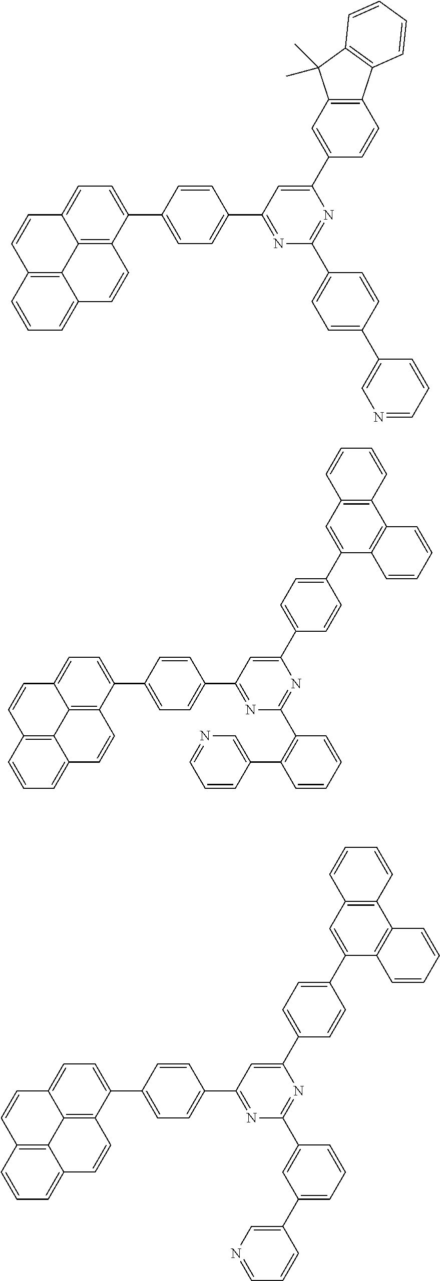

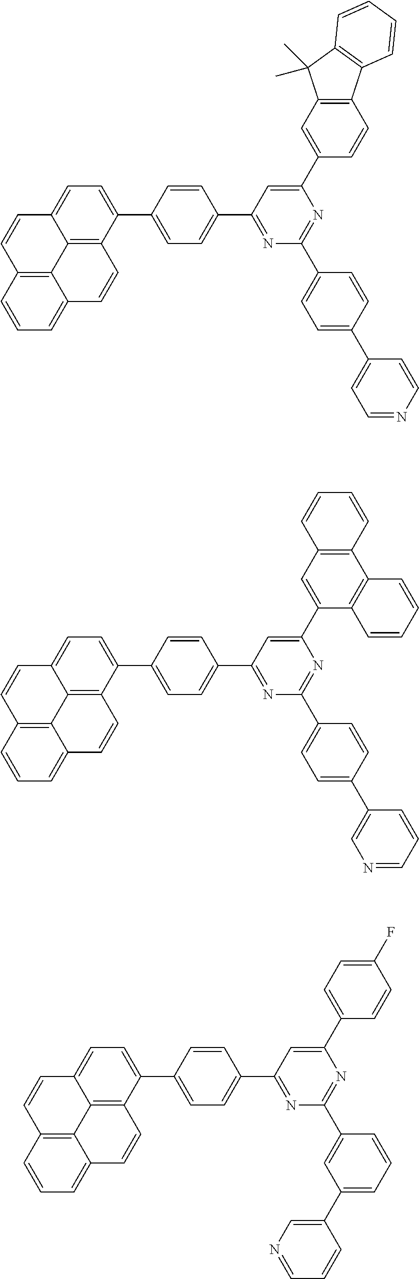 [SMILES: CC1(C)c2ccccc2-c2ccc(-c3cc(-c4ccc(-c5ccc6ccc7cccc8ccc5c6c78)cc4)nc(-c4ccc(-c5ccncc5)cc4)n3)cc21 ; c1cncc(-c2ccc(-c3nc(-c4ccc(-c5ccc6ccc7cccc8ccc5c6c78)cc4)cc(-c4cc5ccccc5c5ccccc45)n3)cc2)c1 ; Fc1ccc(-c2cc(-c3ccc(-c4ccc5ccc6cccc7ccc4c5c67)cc3)nc(-c3cccc(-c4cccnc4)c3)n2)cc1]

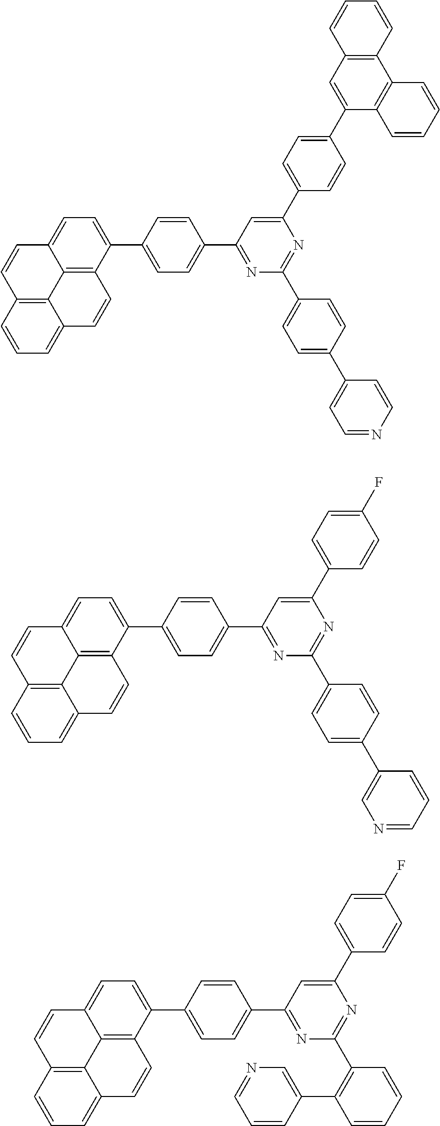

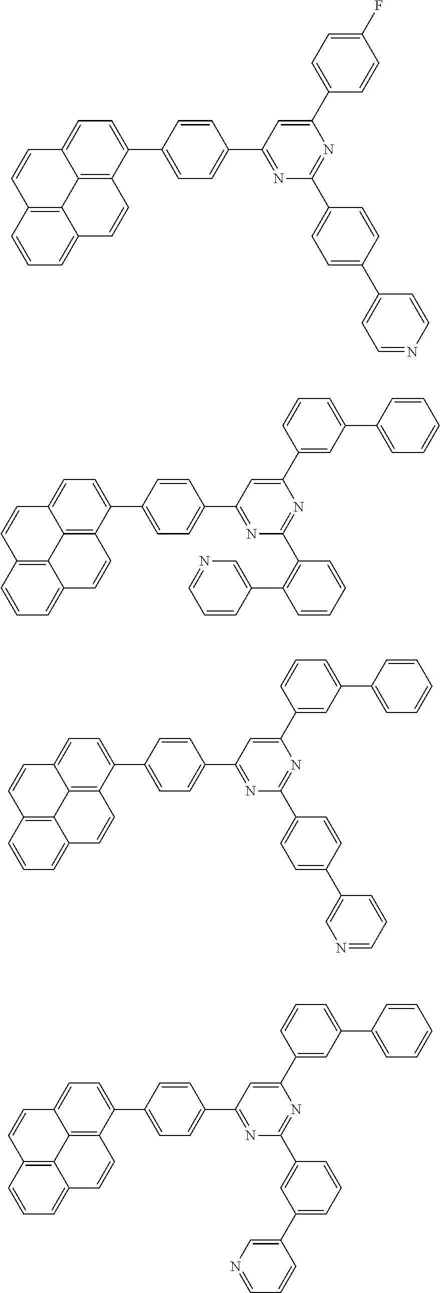 [SMILES: Fc1ccc(-c2cc(-c3ccc(-c4ccc5ccc6cccc7ccc4c5c67)cc3)nc(-c3ccc(-c4ccncc4)cc3)n2)cc1 ; c1ccc(-c2cccc(-c3cc(-c4ccc(-c5ccc6ccc7cccc8ccc5c6c78)cc4)nc(-c4ccccc4-c4cccnc4)n3)c2)cc1 ; c1ccc(-c2cccc(-c3cc(-c4ccc(-c5ccc6ccc7cccc8ccc5c6c78)cc4)nc(-c4ccc(-c5cccnc5)cc4)n3)c2)cc1 ; c1ccc(-c2cccc(-c3cc(-c4ccc(-c5ccc6ccc7cccc8ccc5c6c78)cc4)nc(-c4cccc(-c5cccnc5)c4)n3)c2)cc1]

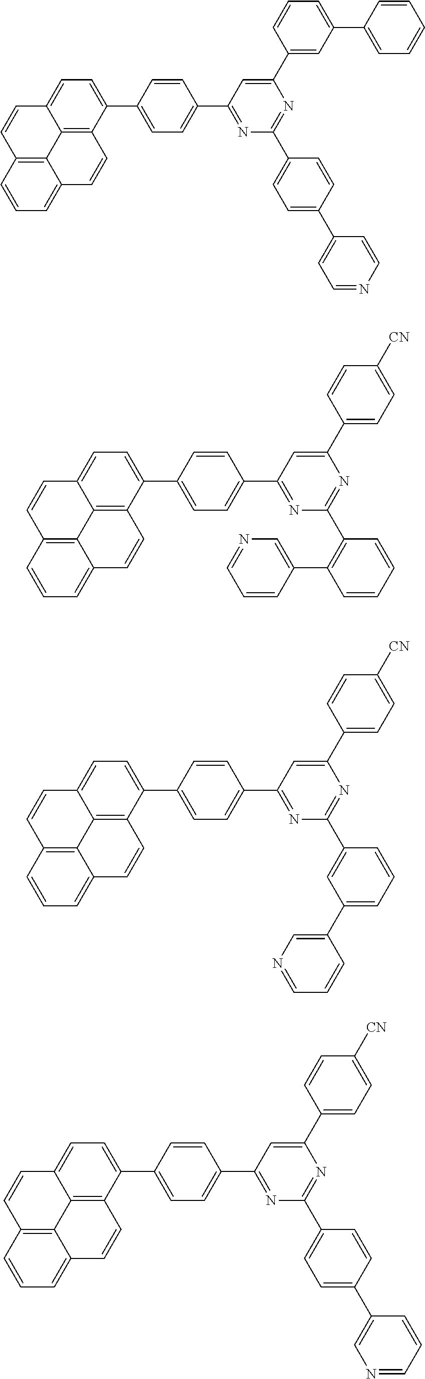 [SMILES: c1ccc(-c2cccc(-c3cc(-c4ccc(-c5ccc6ccc7cccc8ccc5c6c78)cc4)nc(-c4ccc(-c5ccncc5)cc4)n3)c2)cc1 ; N#Cc1ccc(-c2cc(-c3ccc(-c4ccc5ccc6cccc7ccc4c5c67)cc3)nc(-c3ccccc3-c3cccnc3)n2)cc1 ; N#Cc1ccc(-c2cc(-c3ccc(-c4ccc5ccc6cccc7ccc4c5c67)cc3)nc(-c3cccc(-c4cccnc4)c3)n2)cc1 ; N#Cc1ccc(-c2cc(-c3ccc(-c4ccc5ccc6cccc7ccc4c5c67)cc3)nc(-c3ccc(-c4cccnc4)cc3)n2)cc1]

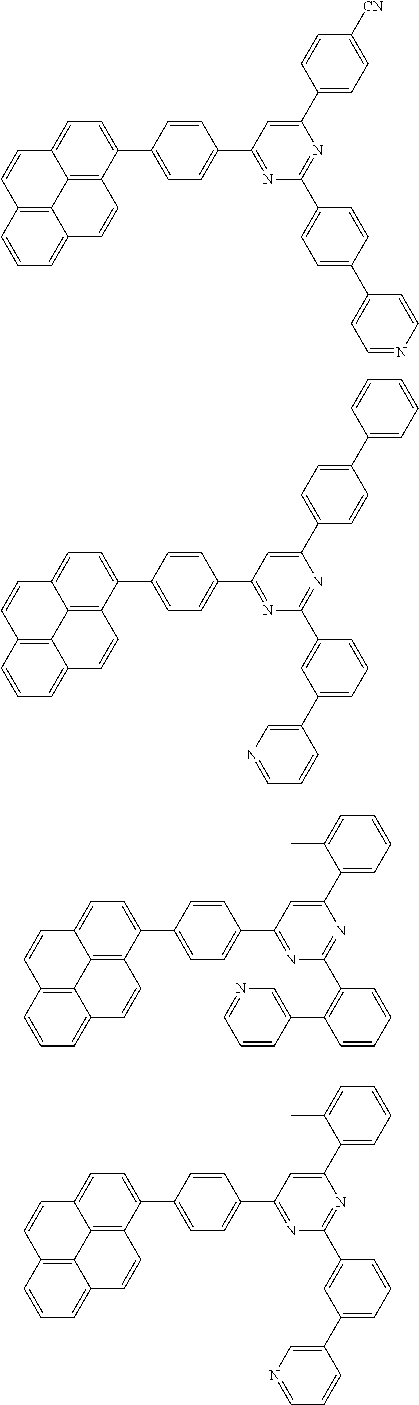

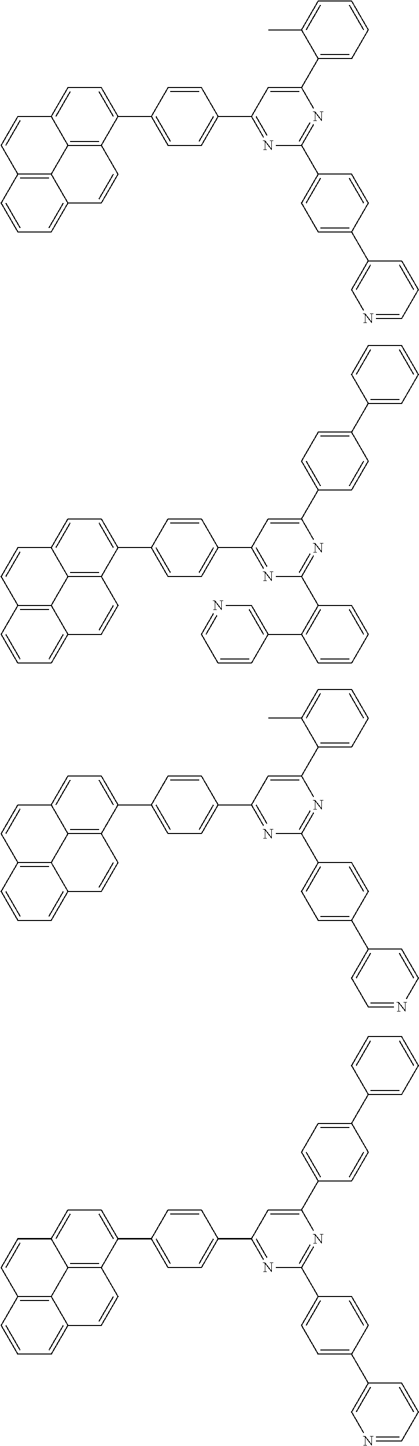

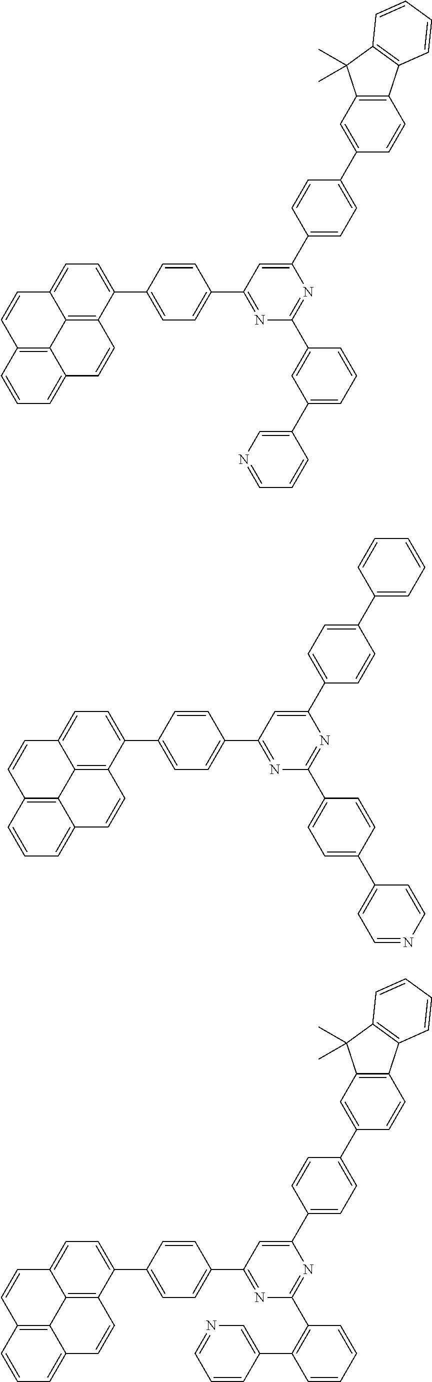

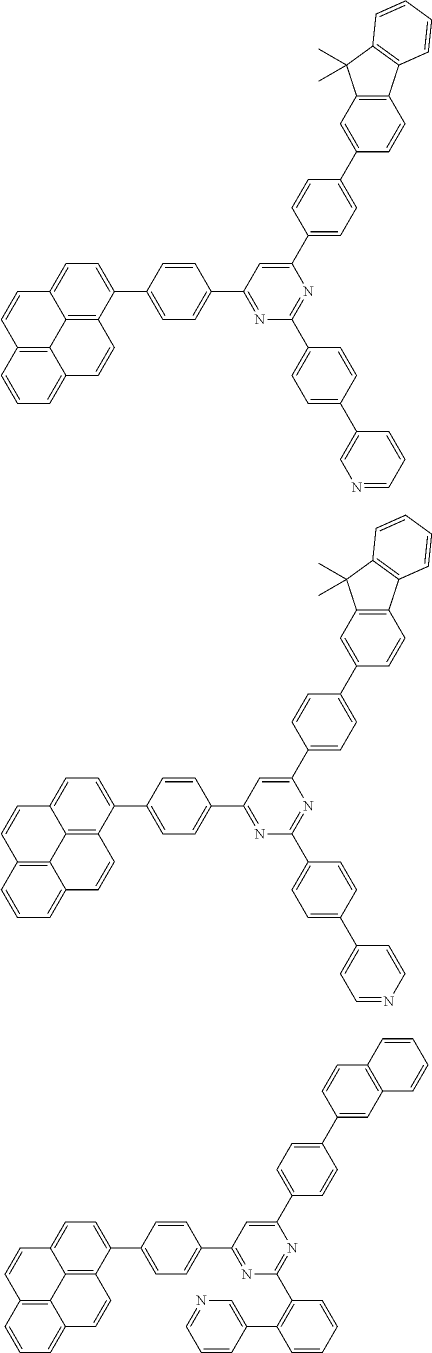

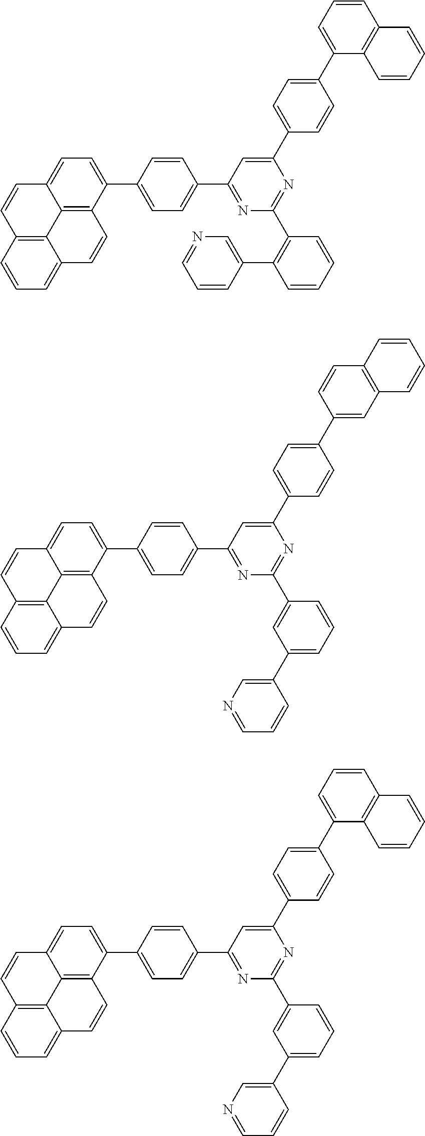 [SMILES: c1cncc(-c2ccccc2-c2nc(-c3ccc(-c4cccc5ccccc45)cc3)cc(-c3ccc(-c4ccc5ccc6cccc7ccc4c5c67)cc3)n2)c1 ; c1cncc(-c2cccc(-c3nc(-c4ccc(-c5ccc6ccccc6c5)cc4)cc(-c4ccc(-c5ccc6ccc7cccc8ccc5c6c78)cc4)n3)c2)c1 ; c1cncc(-c2cccc(-c3nc(-c4ccc(-c5cccc6ccccc56)cc4)cc(-c4ccc(-c5ccc6ccc7cccc8ccc5c6c78)cc4)n3)c2)c1]

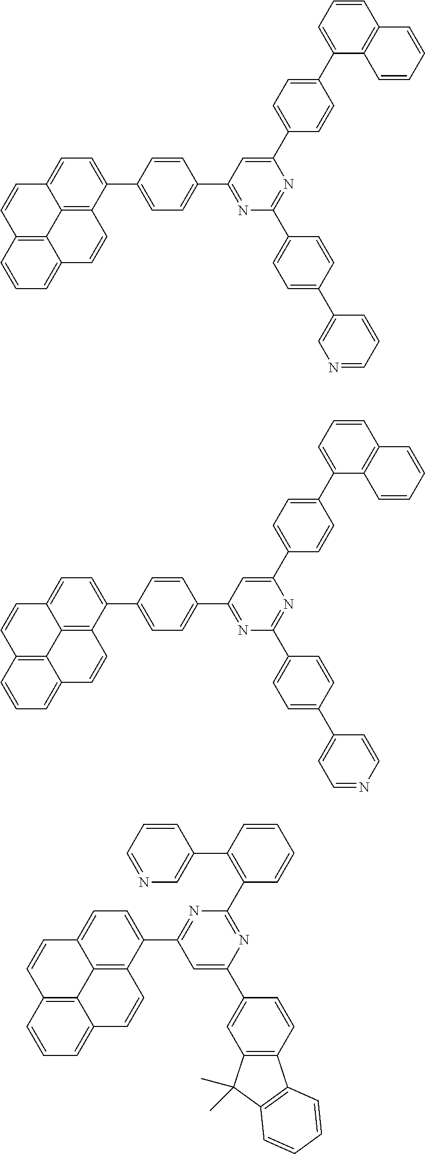 [SMILES: c1cncc(-c2ccc(-c3nc(-c4ccc(-c5cccc6ccccc56)cc4)cc(-c4ccc(-c5ccc6ccc7cccc8ccc5c6c78)cc4)n3)cc2)c1 ; c1ccc2c(-c3ccc(-c4cc(-c5ccc(-c6ccc7ccc8cccc9ccc6c7c89)cc5)nc(-c5ccc(-c6ccncc6)cc5)n4)cc3)cccc2c1 ; CC1(C)c2ccccc2-c2ccc(-c3cc(-c4ccc5ccc6cccc7ccc4c5c67)nc(-c4ccccc4-c4cccnc4)n3)cc21]

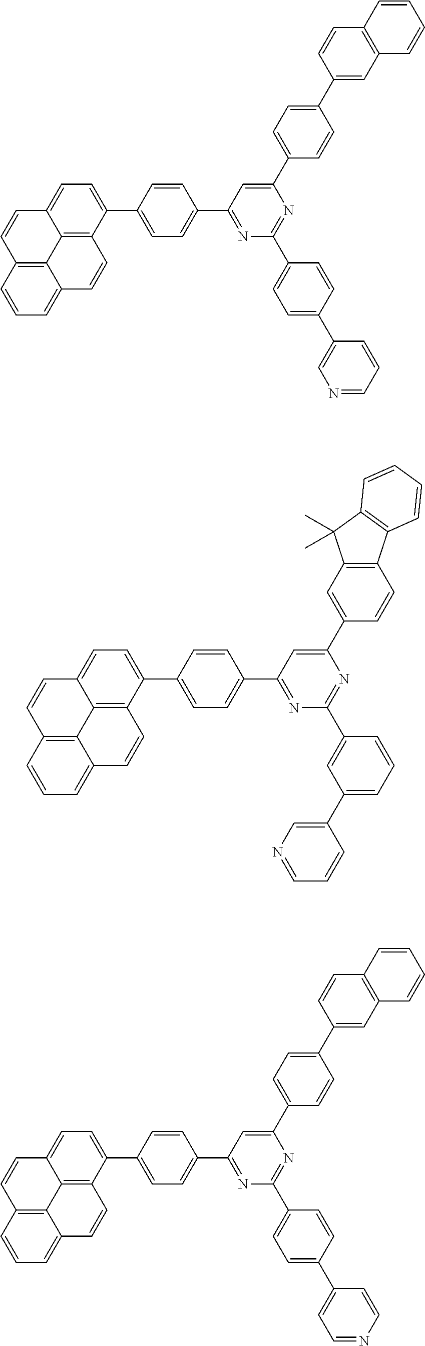 [SMILES: c1cncc(-c2ccc(-c3nc(-c4ccc(-c5ccc6ccccc6c5)cc4)cc(-c4ccc(-c5ccc6ccc7cccc8ccc5c6c78)cc4)n3)cc2)c1 ; CC1(C)c2ccccc2-c2ccc(-c3cc(-c4ccc(-c5ccc6ccc7cccc8ccc5c6c78)cc4)nc(-c4cccc(-c5cccnc5)c4)n3)cc21 ; c1ccc2cc(-c3ccc(-c4cc(-c5ccc(-c6ccc7ccc8cccc9ccc6c7c89)cc5)nc(-c5ccc(-c6ccncc6)cc5)n4)cc3)ccc2c1]

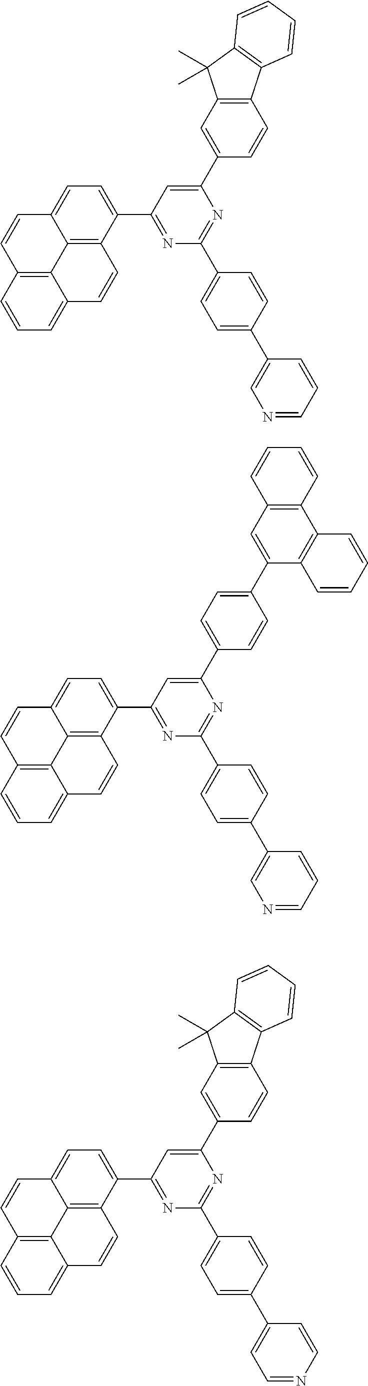

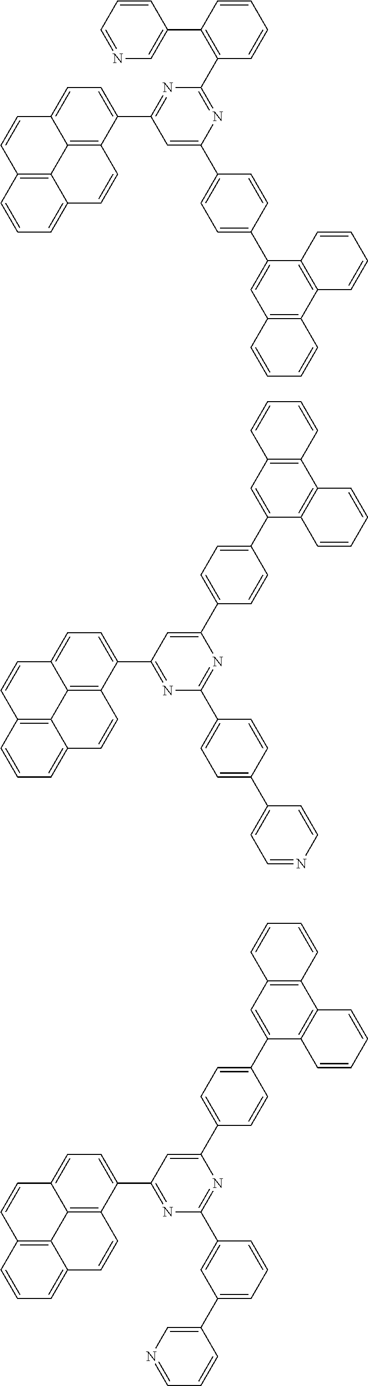

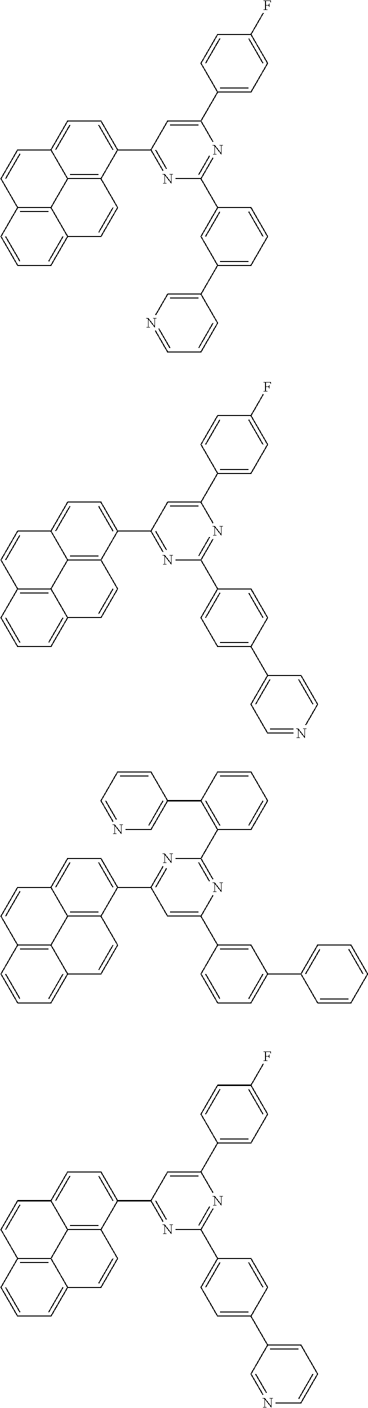

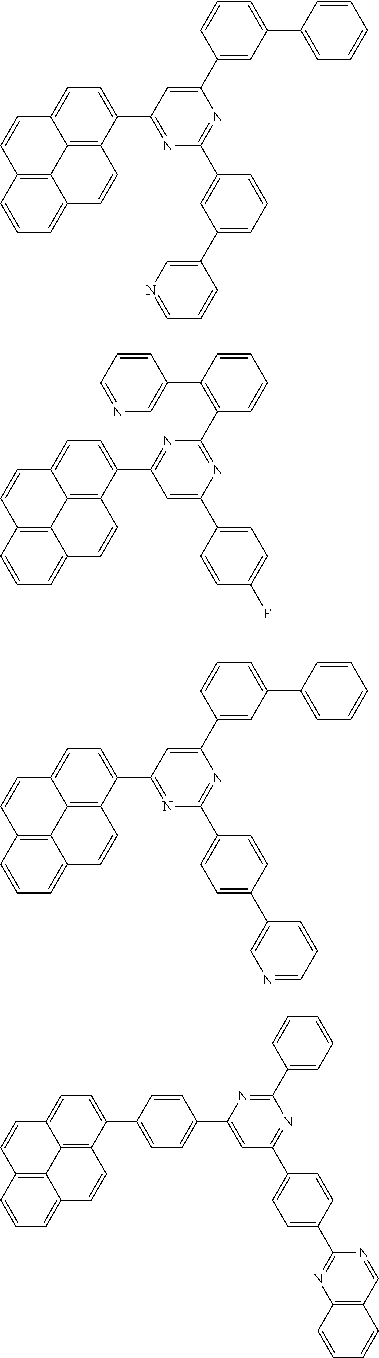

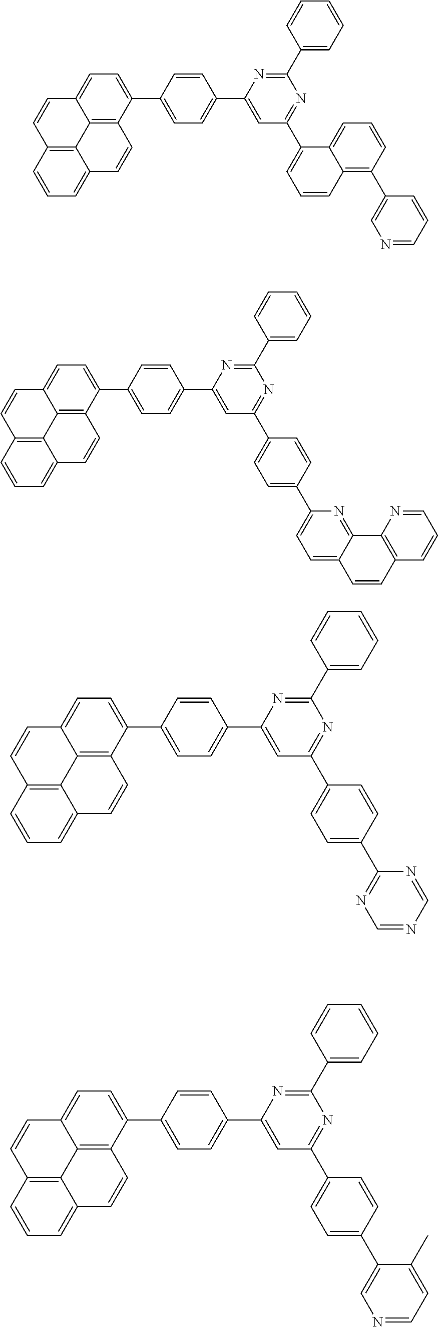

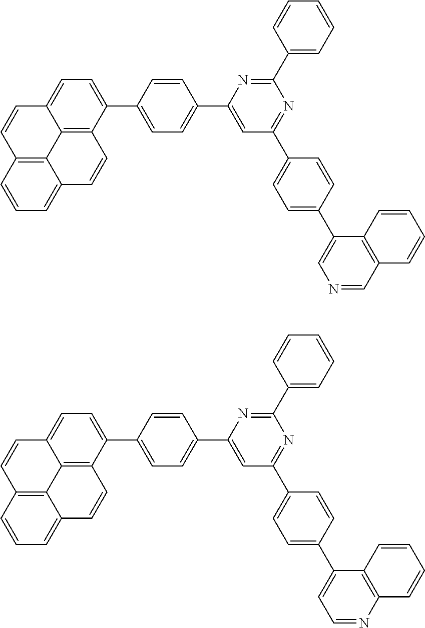 [SMILES: c1ccc(-c2nc(-c3ccc(-c4cncc5ccccc45)cc3)cc(-c3ccc(-c4ccc5ccc6cccc7ccc4c5c67)cc3)n2)cc1 ; c1ccc(-c2nc(-c3ccc(-c4ccnc5ccccc45)cc3)cc(-c3ccc(-c4ccc5ccc6cccc7ccc4c5c67)cc3)n2)cc1]

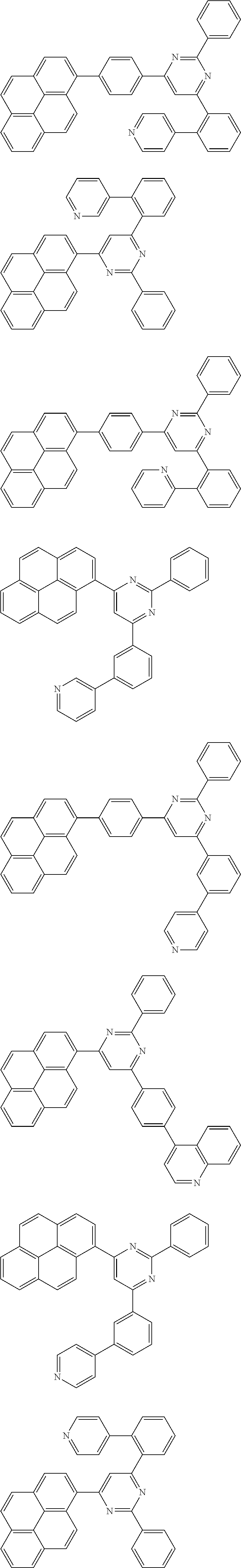

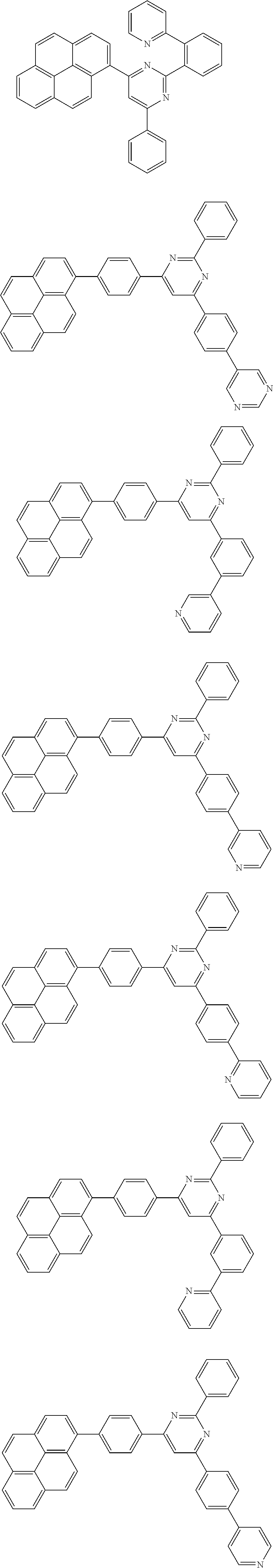

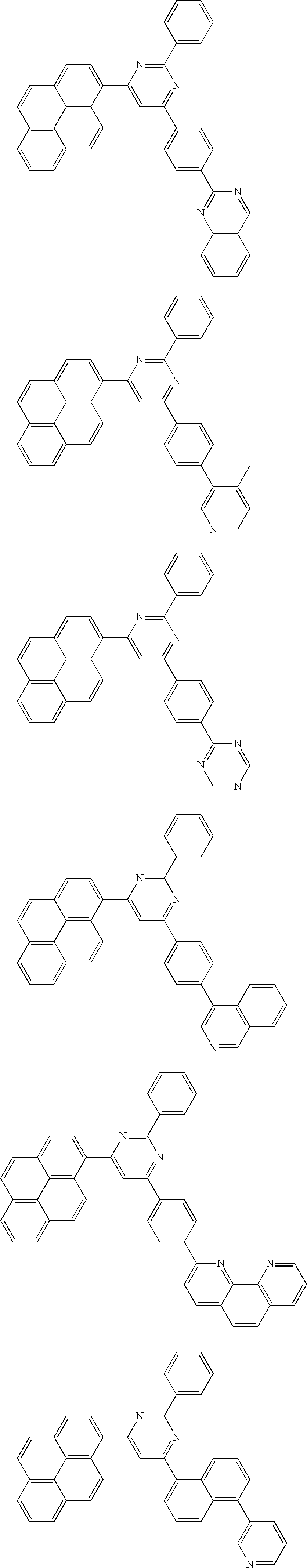

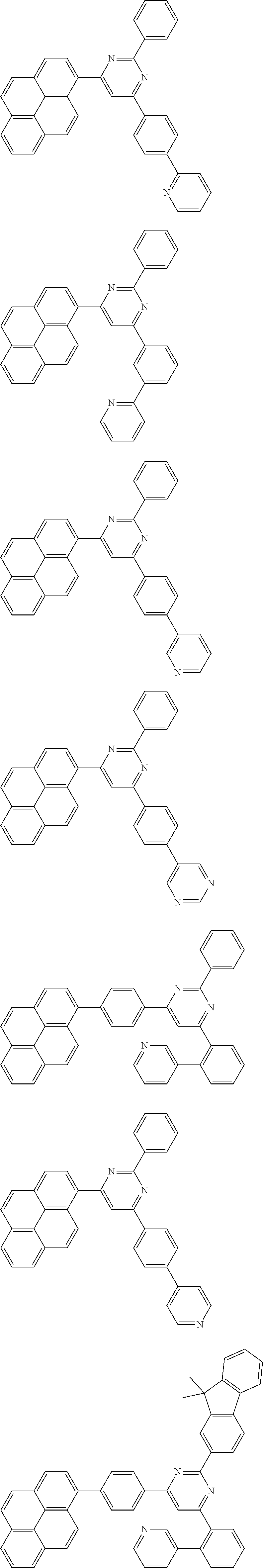

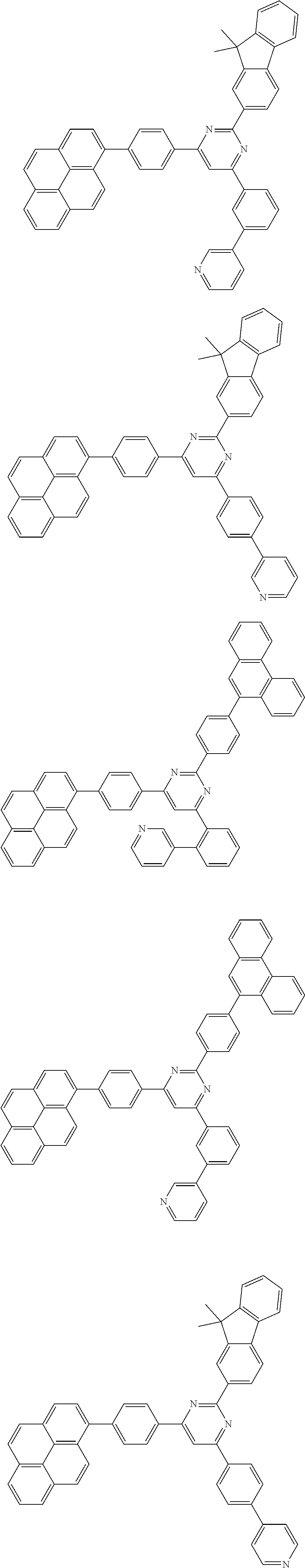

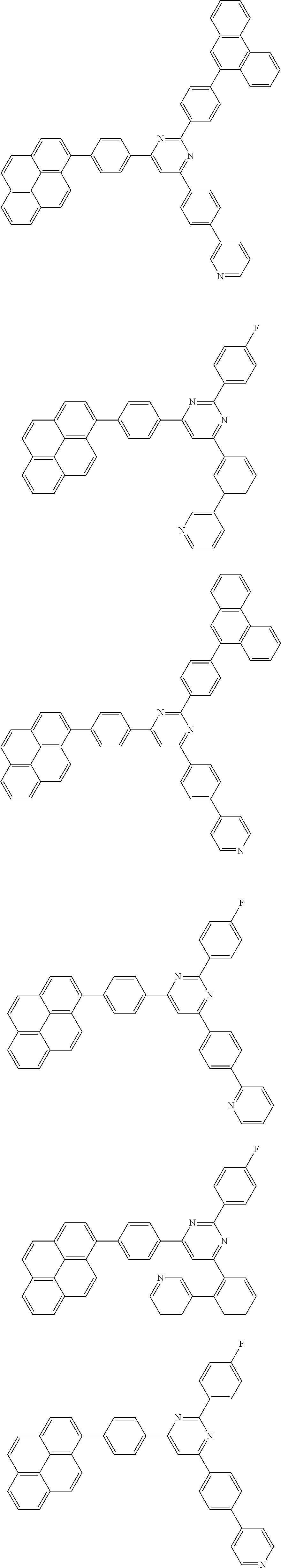

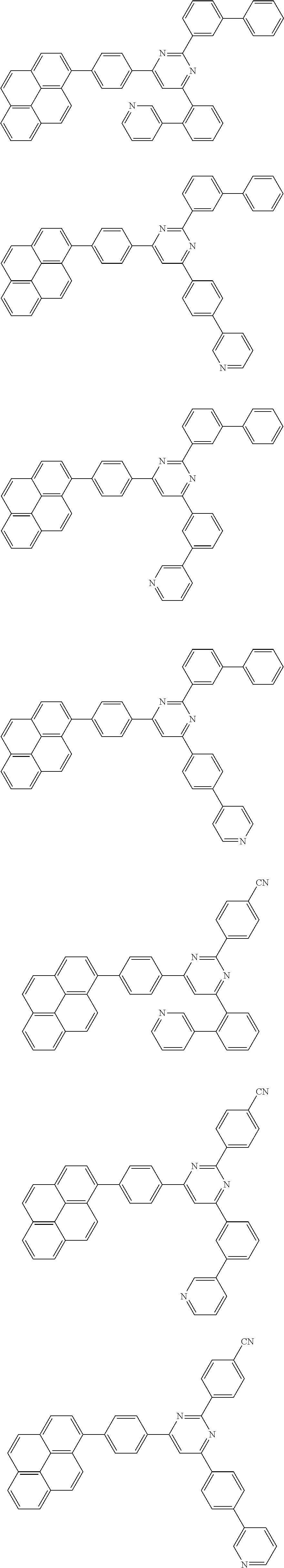

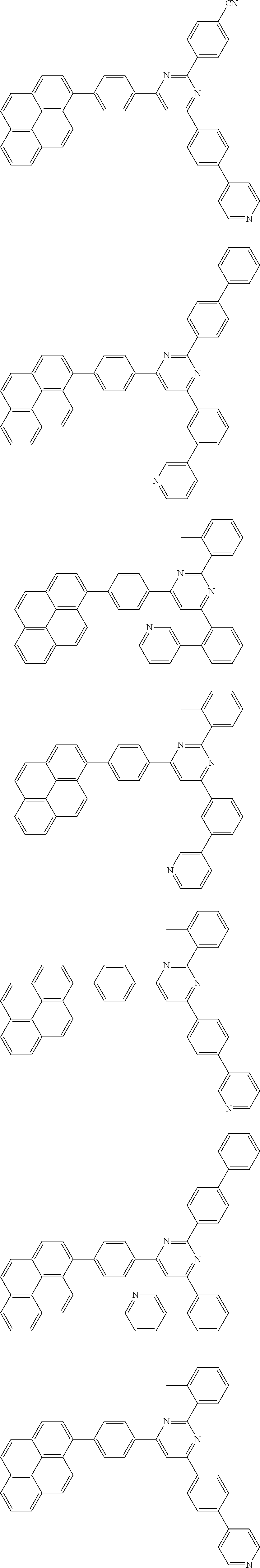

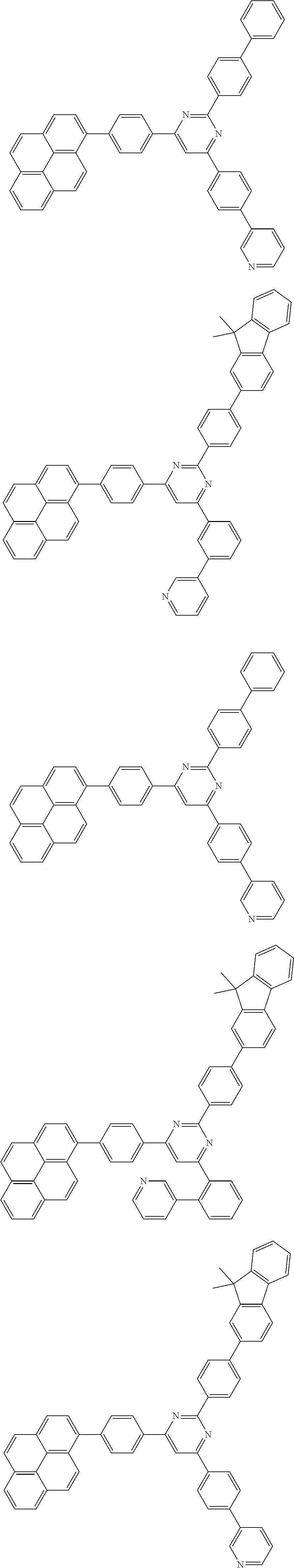

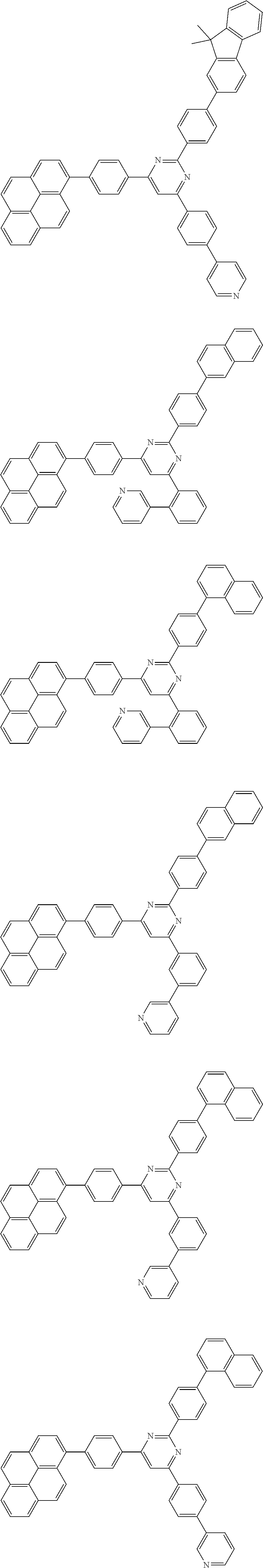

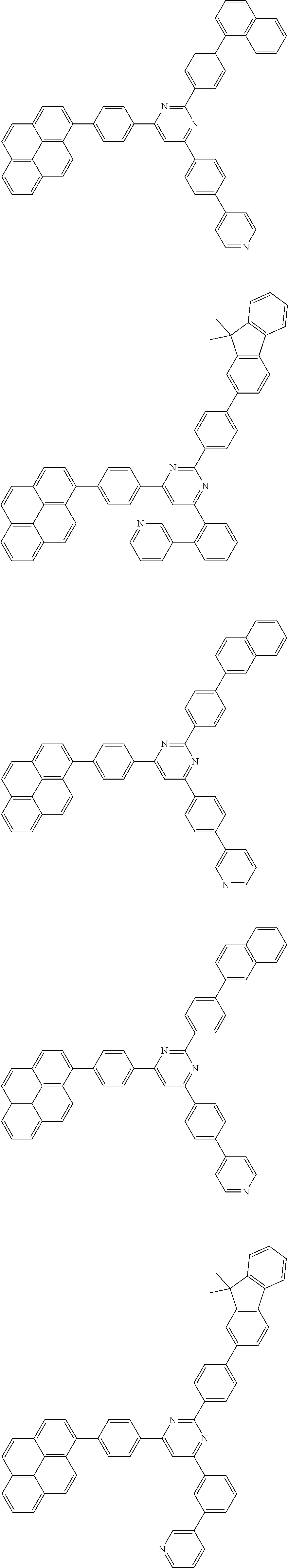

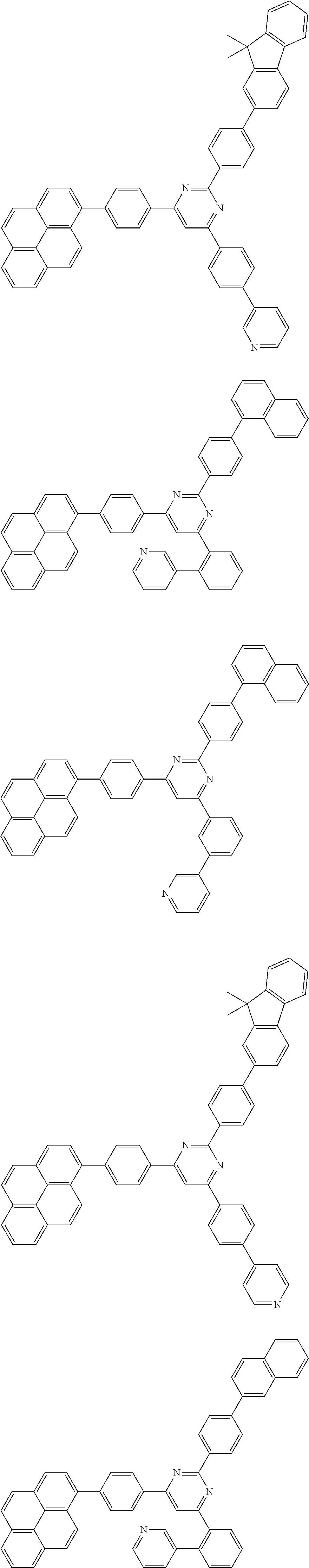

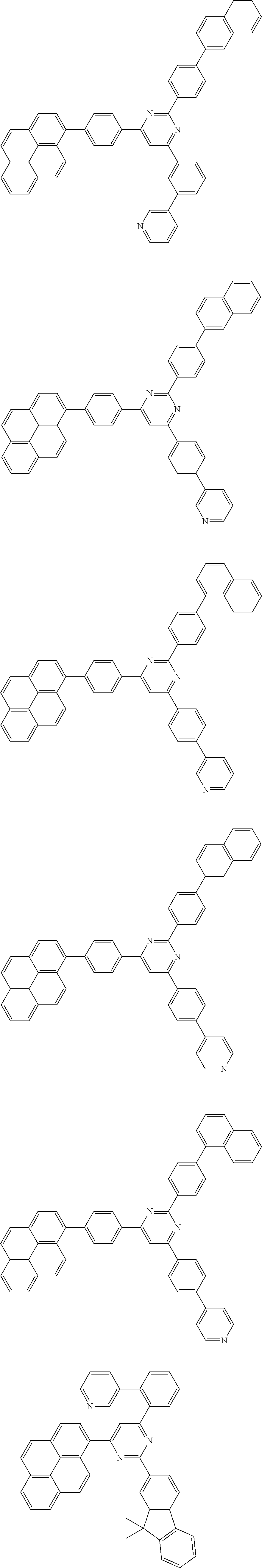

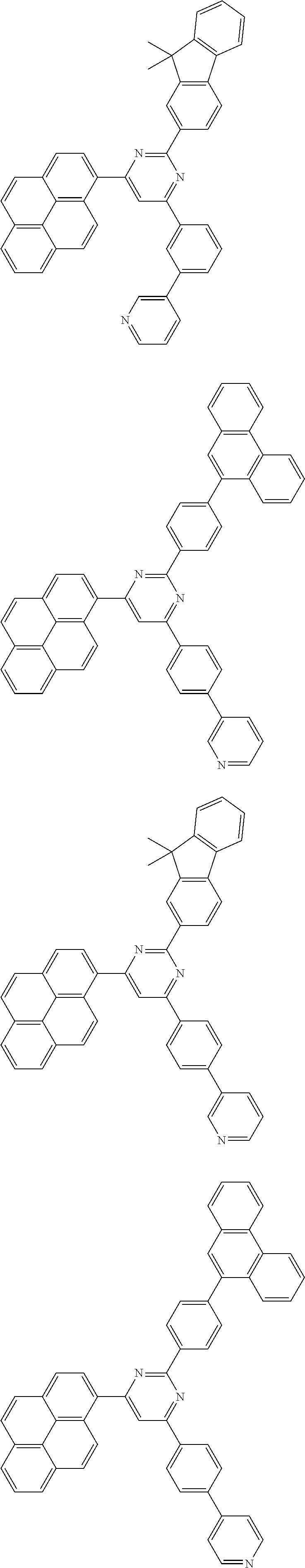

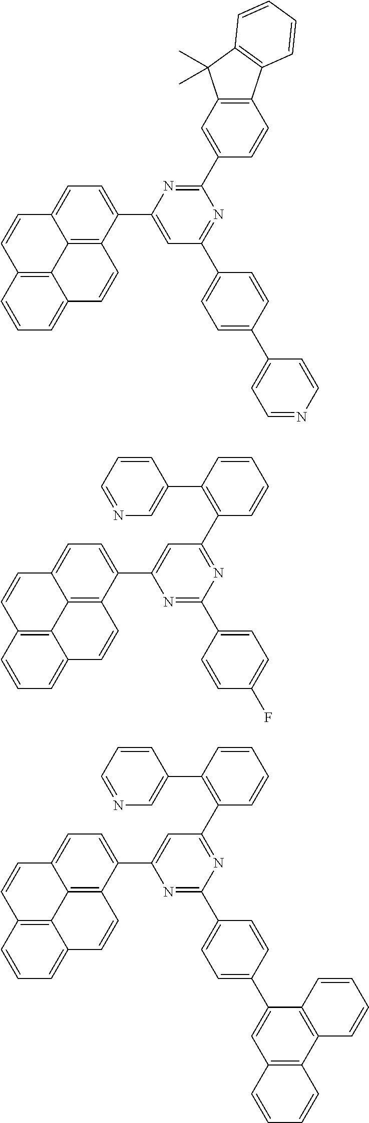

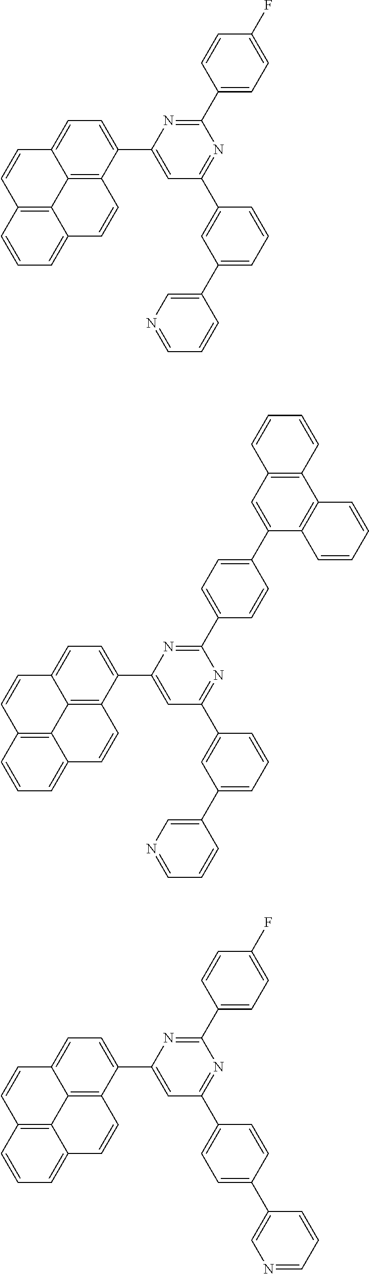 [SMILES: Fc1ccc(-c2nc(-c3cccc(-c4cccnc4)c3)cc(-c3ccc4ccc5cccc6ccc3c4c56)n2)cc1 ; c1cncc(-c2cccc(-c3cc(-c4ccc5ccc6cccc7ccc4c5c67)nc(-c4ccc(-c5cc6ccccc6c6ccccc56)cc4)n3)c2)c1 ; Fc1ccc(-c2nc(-c3ccc(-c4cccnc4)cc3)cc(-c3ccc4ccc5cccc6ccc3c4c56)n2)cc1]

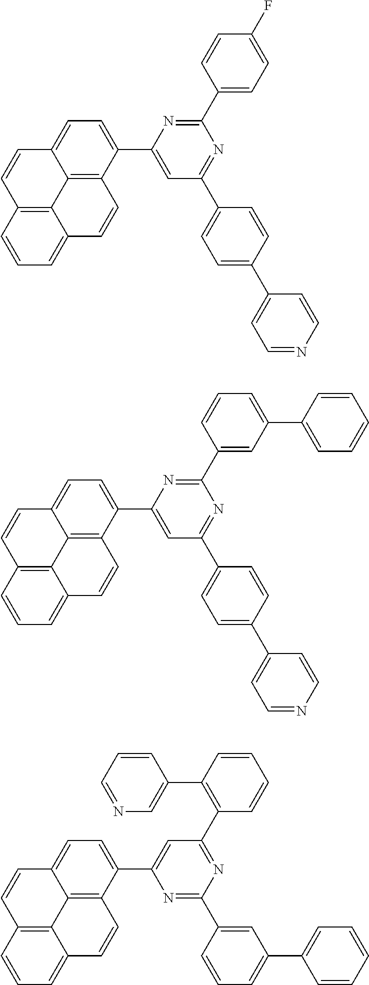

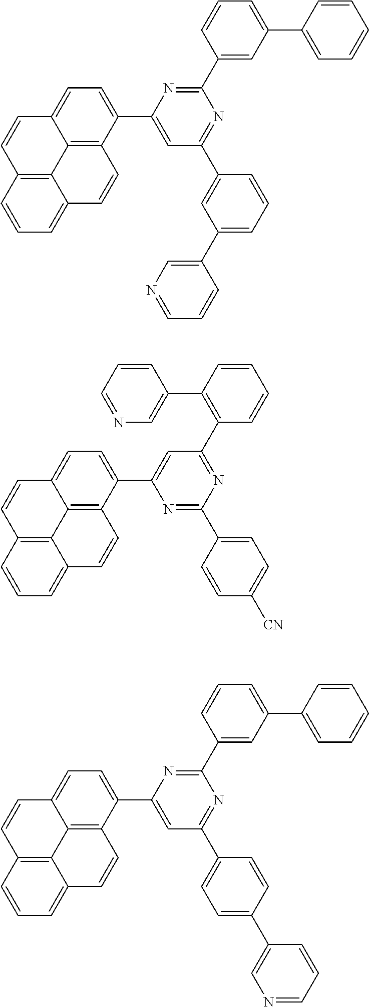 [SMILES: c1ccc(-c2cccc(-c3nc(-c4cccc(-c5cccnc5)c4)cc(-c4ccc5ccc6cccc7ccc4c5c67)n3)c2)cc1 ; N#Cc1ccc(-c2nc(-c3ccccc3-c3cccnc3)cc(-c3ccc4ccc5cccc6ccc3c4c56)n2)cc1 ; c1ccc(-c2cccc(-c3nc(-c4ccc(-c5cccnc5)cc4)cc(-c4ccc5ccc6cccc7ccc4c5c67)n3)c2)cc1]

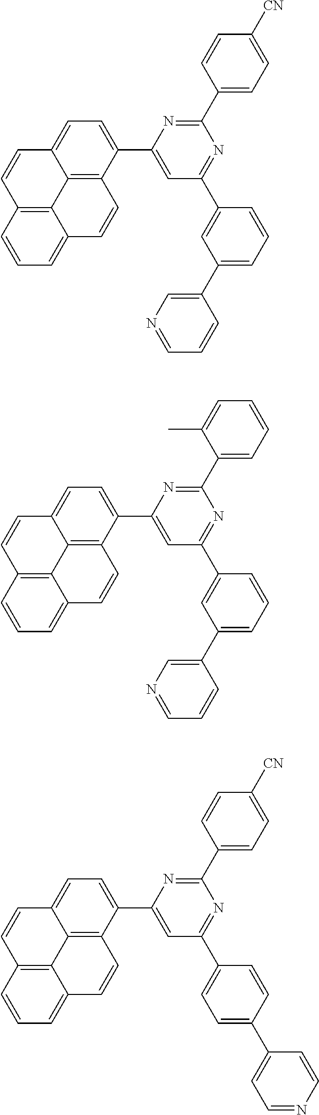 [SMILES: N#Cc1ccc(-c2nc(-c3cccc(-c4cccnc4)c3)cc(-c3ccc4ccc5cccc6ccc3c4c56)n2)cc1 ; Cc1ccccc1-c1nc(-c2cccc(-c3cccnc3)c2)cc(-c2ccc3ccc4cccc5ccc2c3c45)n1 ; N#Cc1ccc(-c2nc(-c3ccc(-c4ccncc4)cc3)cc(-c3ccc4ccc5cccc6ccc3c4c56)n2)cc1]

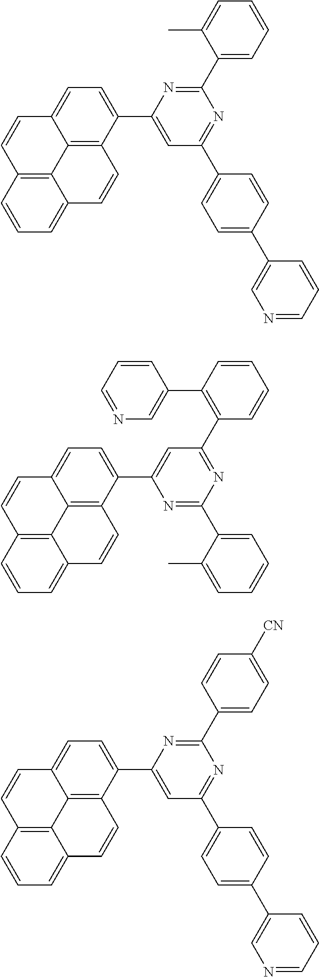

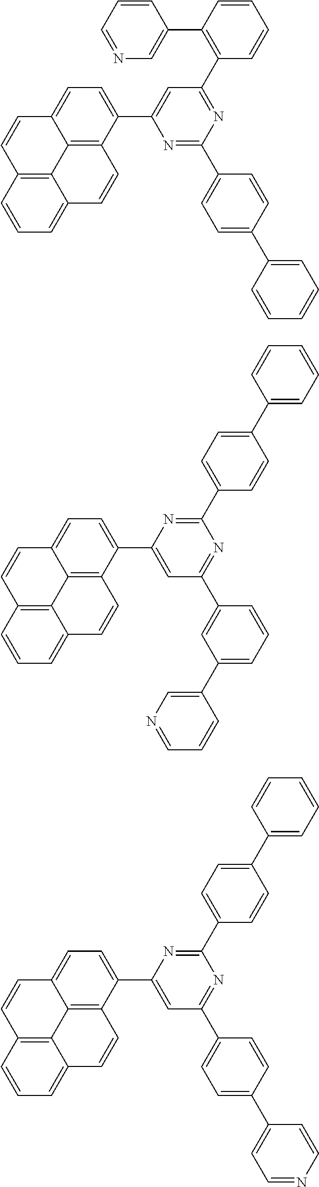

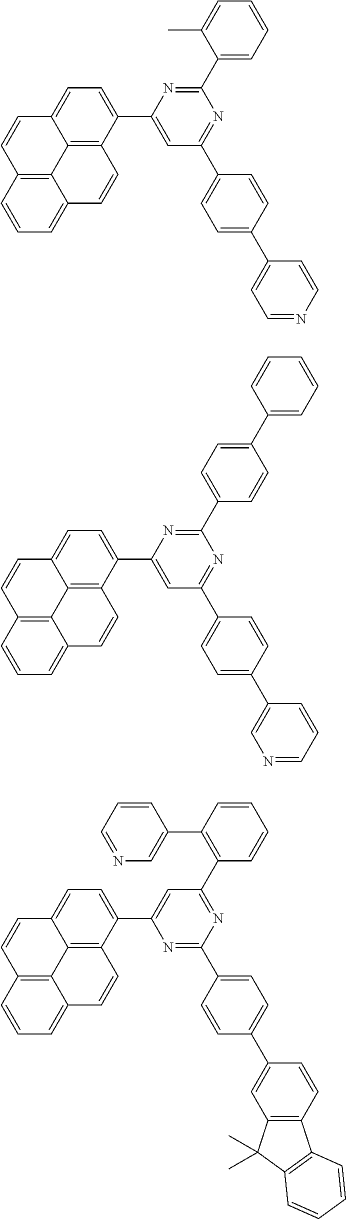

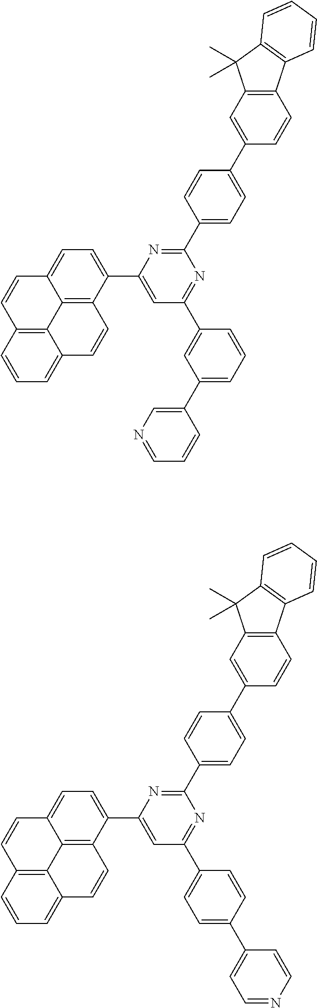 [SMILES: CC1(C)c2ccccc2-c2ccc(-c3ccc(-c4nc(-c5cccc(-c6cccnc6)c5)cc(-c5ccc6ccc7cccc8ccc5c6c78)n4)cc3)cc21 ; CC1(C)c2ccccc2-c2ccc(-c3ccc(-c4nc(-c5ccc(-c6ccncc6)cc5)cc(-c5ccc6ccc7cccc8ccc5c6c78)n4)cc3)cc21]

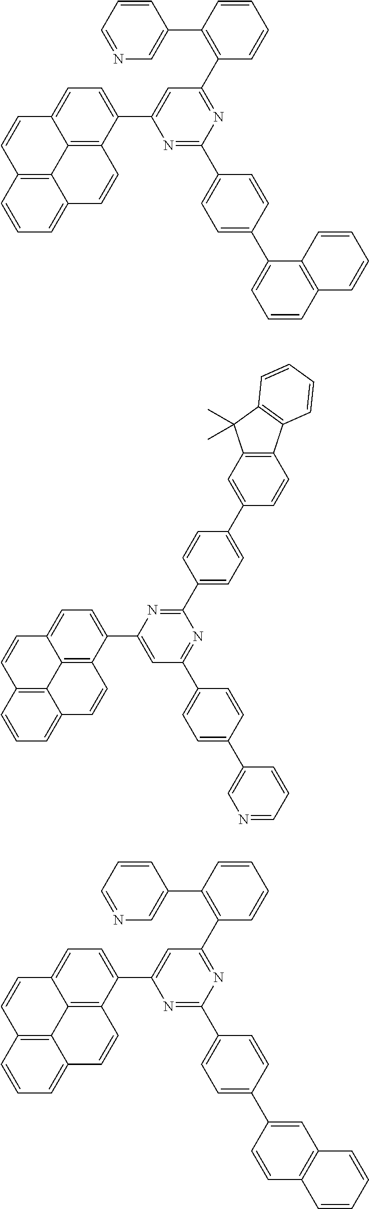

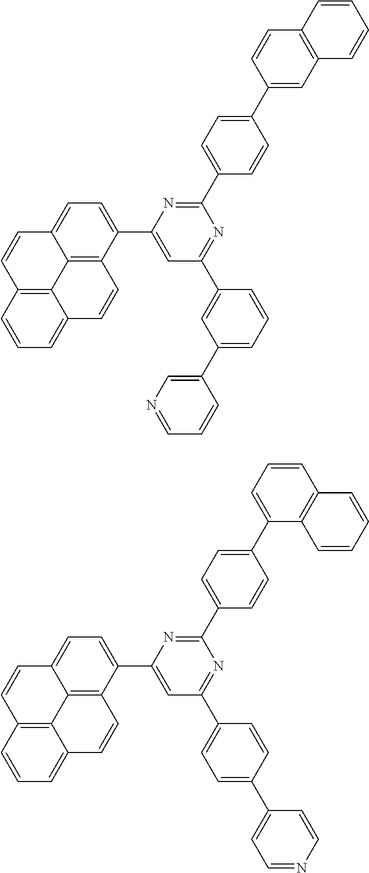 [SMILES: c1cncc(-c2cccc(-c3cc(-c4ccc5ccc6cccc7ccc4c5c67)nc(-c4ccc(-c5ccc6ccccc6c5)cc4)n3)c2)c1 ; c1ccc2c(-c3ccc(-c4nc(-c5ccc(-c6ccncc6)cc5)cc(-c5ccc6ccc7cccc8ccc5c6c78)n4)cc3)cccc2c1]

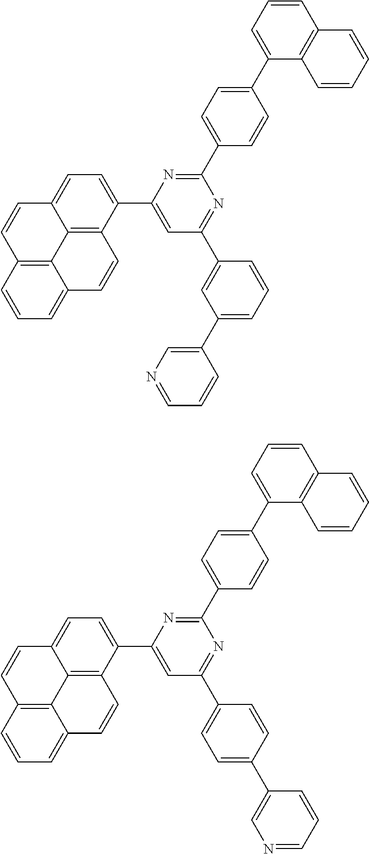 [SMILES: c1cncc(-c2cccc(-c3cc(-c4ccc5ccc6cccc7ccc4c5c67)nc(-c4ccc(-c5cccc6ccccc56)cc4)n3)c2)c1 ; c1cncc(-c2ccc(-c3cc(-c4ccc5ccc6cccc7ccc4c5c67)nc(-c4ccc(-c5cccc6ccccc56)cc4)n3)cc2)c1]

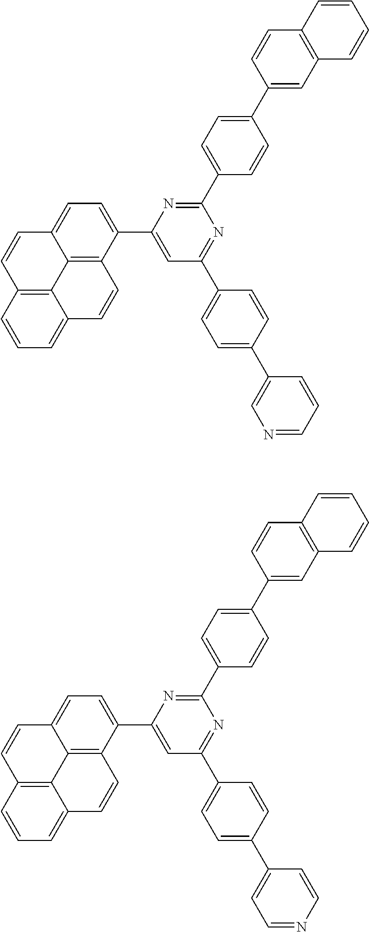 [SMILES: c1cncc(-c2ccc(-c3cc(-c4ccc5ccc6cccc7ccc4c5c67)nc(-c4ccc(-c5ccc6ccccc6c5)cc4)n3)cc2)c1 ; c1ccc2cc(-c3ccc(-c4nc(-c5ccc(-c6ccncc6)cc5)cc(-c5ccc6ccc7cccc8ccc5c6c78)n4)cc3)ccc2c1]

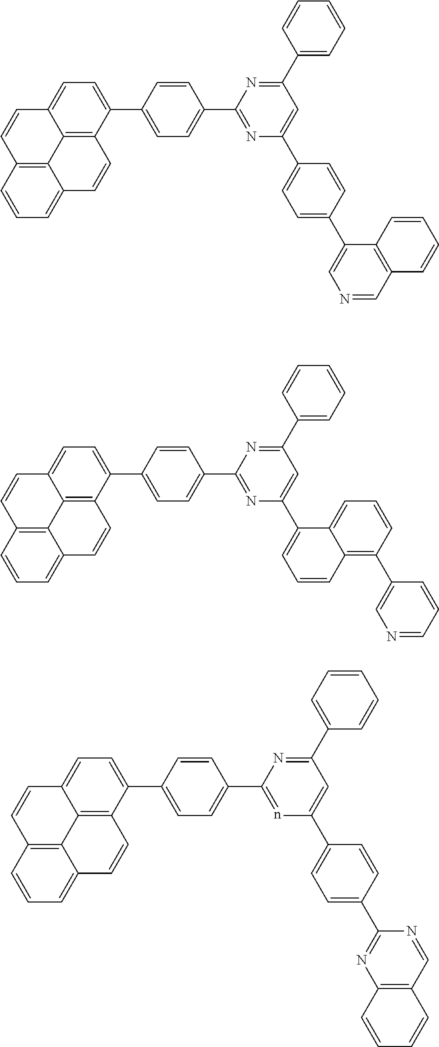

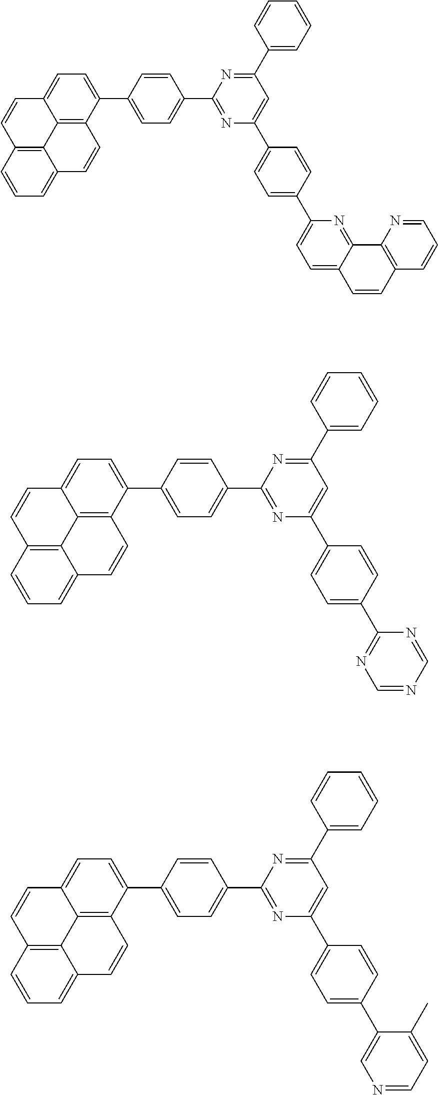 [SMILES: c1ccc(-c2cc(-c3ccc(-c4ccc5ccc6cccnc6c5n4)cc3)nc(-c3ccc(-c4ccc5ccc6cccc7ccc4c5c67)cc3)n2)cc1 ; c1ccc(-c2cc(-c3ccc(-c4ncncn4)cc3)nc(-c3ccc(-c4ccc5ccc6cccc7ccc4c5c67)cc3)n2)cc1 ; Cc1ccncc1-c1ccc(-c2cc(-c3ccccc3)nc(-c3ccc(-c4ccc5ccc6cccc7ccc4c5c67)cc3)n2)cc1]

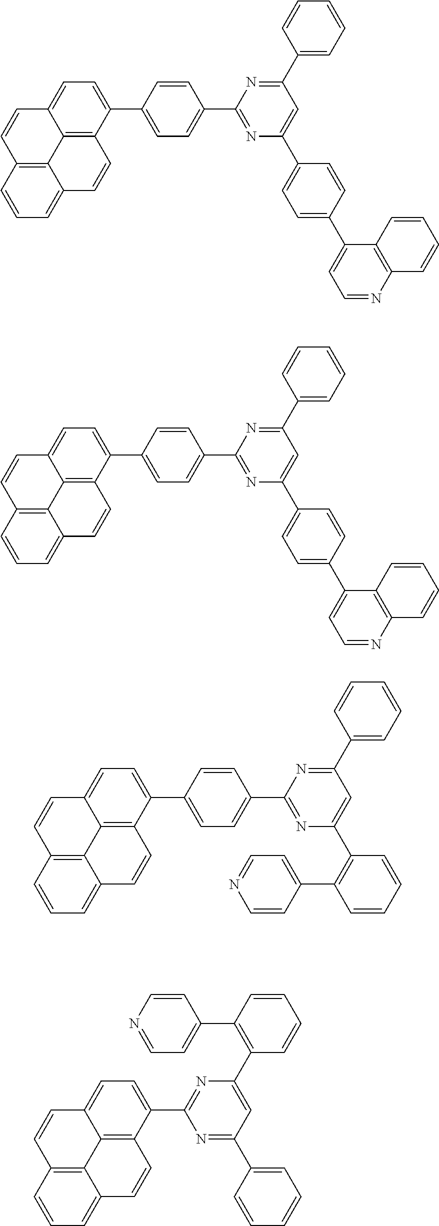

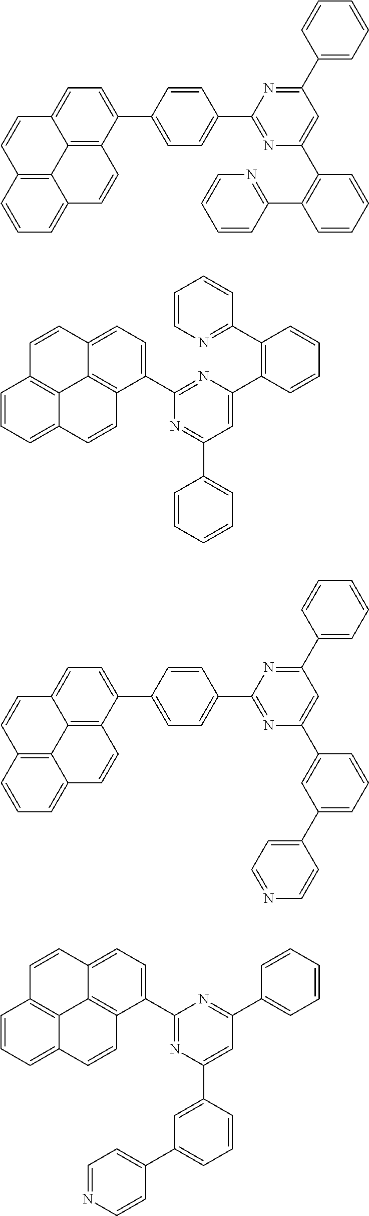

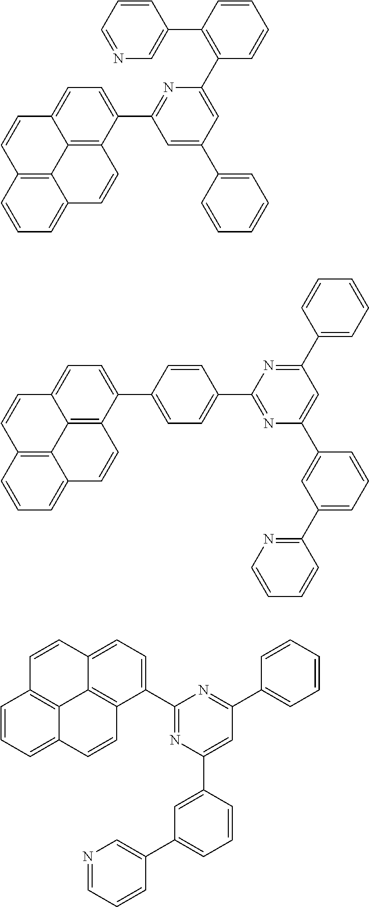

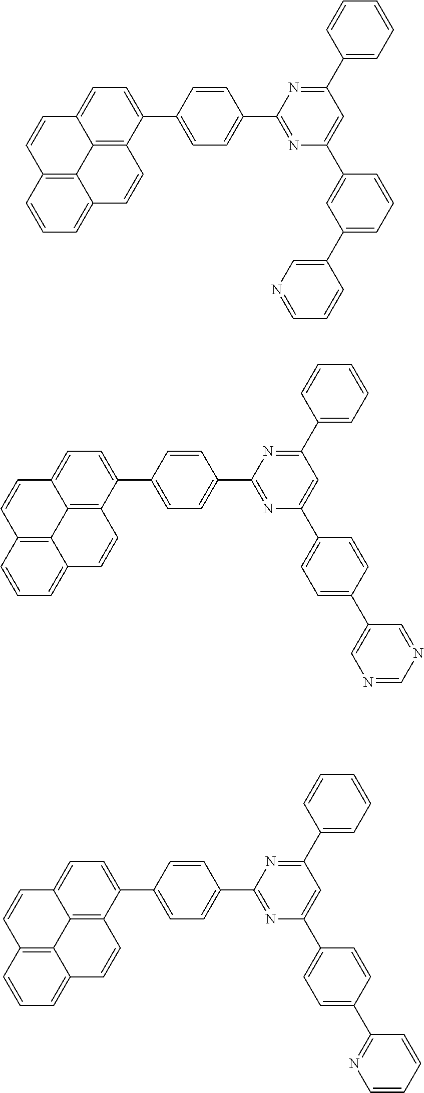 [SMILES: c1ccc(-c2cc(-c3cccc(-c4cccnc4)c3)nc(-c3ccc(-c4ccc5ccc6cccc7ccc4c5c67)cc3)n2)cc1 ; c1ccc(-c2cc(-c3ccc(-c4cncnc4)cc3)nc(-c3ccc(-c4ccc5ccc6cccc7ccc4c5c67)cc3)n2)cc1 ; c1ccc(-c2cc(-c3ccc(-c4ccccn4)cc3)nc(-c3ccc(-c4ccc5ccc6cccc7ccc4c5c67)cc3)n2)cc1]

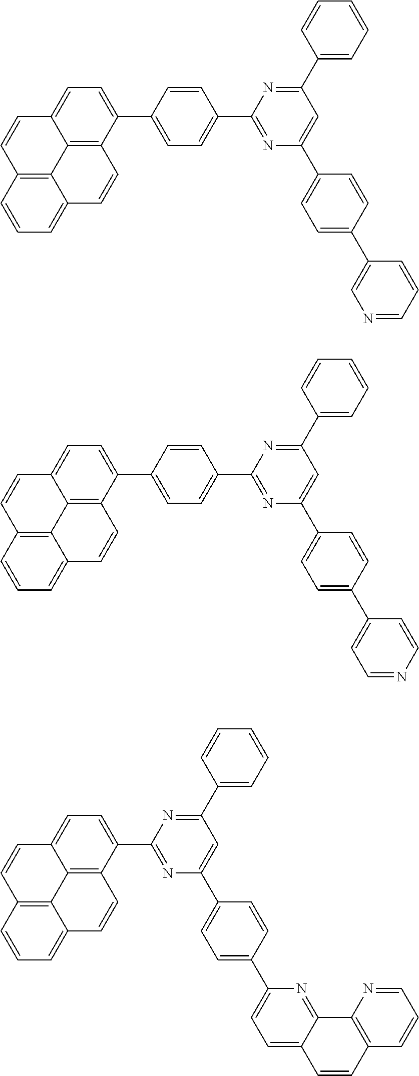

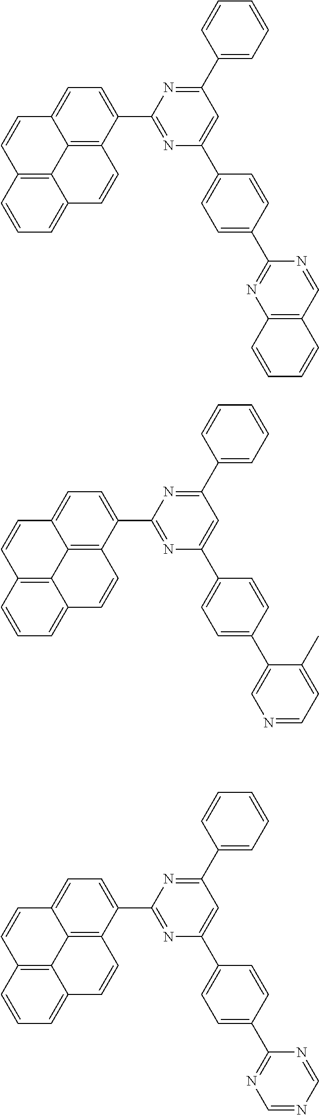

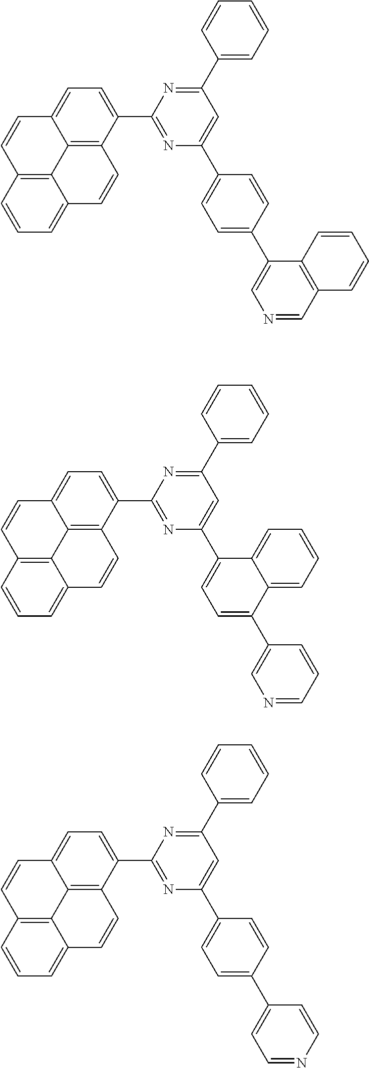

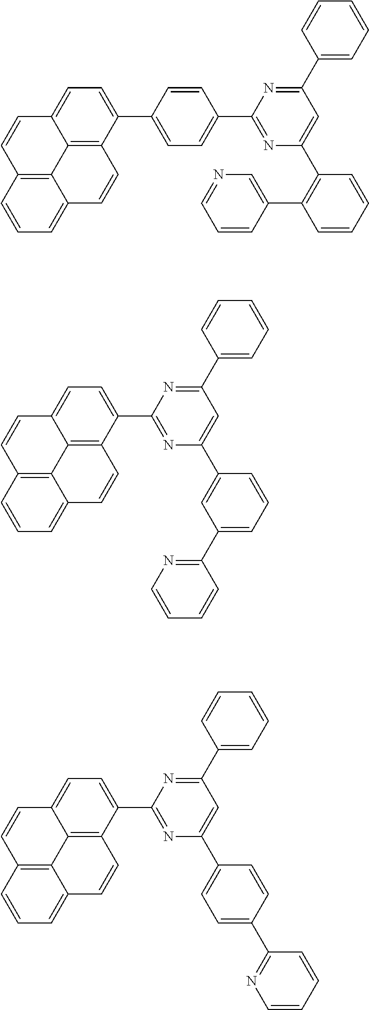 [SMILES: c1ccc(-c2cc(-c3ccccc3-c3cccnc3)nc(-c3ccc(-c4ccc5ccc6cccc7ccc4c5c67)cc3)n2)cc1 ; c1ccc(-c2cc(-c3cccc(-c4ccccn4)c3)nc(-c3ccc4ccc5cccc6ccc3c4c56)n2)cc1 ; c1ccc(-c2cc(-c3ccc(-c4ccccn4)cc3)nc(-c3ccc4ccc5cccc6ccc3c4c56)n2)cc1]

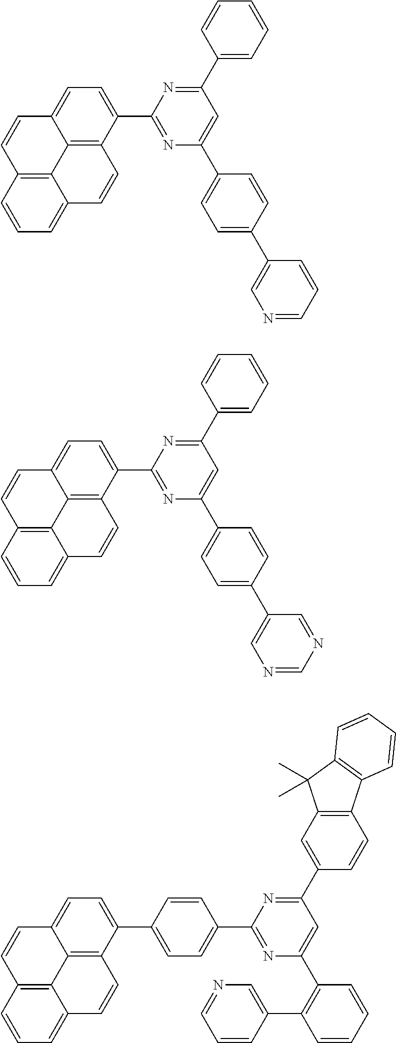

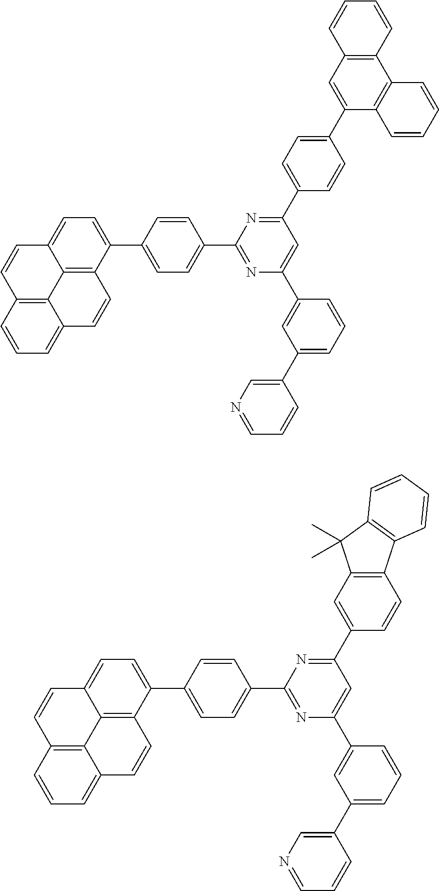 [SMILES: c1cncc(-c2cccc(-c3cc(-c4ccc(-c5cc6ccccc6c6ccccc56)cc4)nc(-c4ccc(-c5ccc6ccc7cccc8ccc5c6c78)cc4)n3)c2)c1 ; CC1(C)c2ccccc2-c2ccc(-c3cc(-c4cccc(-c5cccnc5)c4)nc(-c4ccc(-c5ccc6ccc7cccc8ccc5c6c78)cc4)n3)cc21]

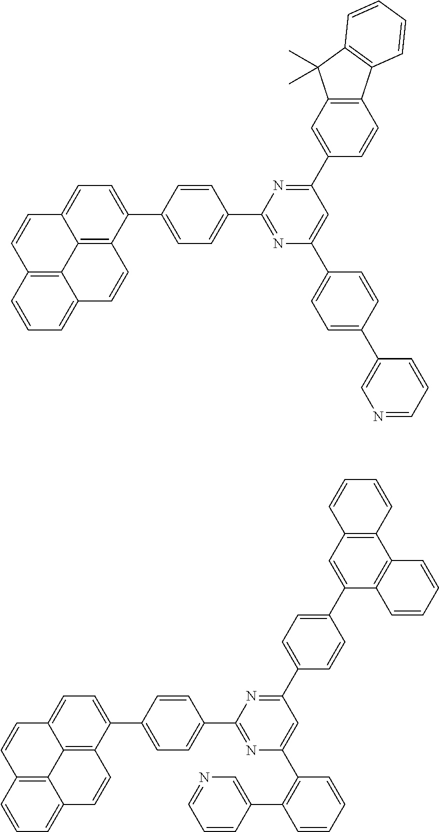 [SMILES: CC1(C)c2ccccc2-c2ccc(-c3cc(-c4ccc(-c5cccnc5)cc4)nc(-c4ccc(-c5ccc6ccc7cccc8ccc5c6c78)cc4)n3)cc21 ; c1cncc(-c2ccccc2-c2cc(-c3ccc(-c4cc5ccccc5c5ccccc45)cc3)nc(-c3ccc(-c4ccc5ccc6cccc7ccc4c5c67)cc3)n2)c1]

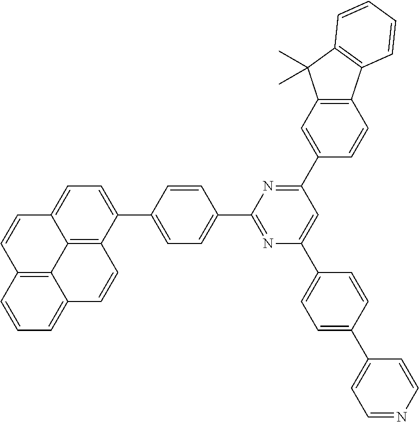 [SMILES: CC1(C)c2ccccc2-c2ccc(-c3cc(-c4ccc(-c5ccncc5)cc4)nc(-c4ccc(-c5ccc6ccc7cccc8ccc5c6c78)cc4)n3)cc21]

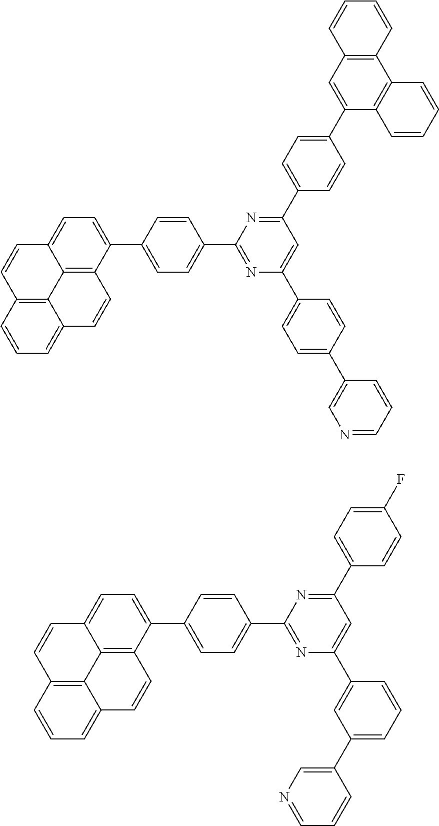 [SMILES: c1cncc(-c2ccc(-c3cc(-c4ccc(-c5cc6ccccc6c6ccccc56)cc4)nc(-c4ccc(-c5ccc6ccc7cccc8ccc5c6c78)cc4)n3)cc2)c1 ; Fc1ccc(-c2cc(-c3cccc(-c4cccnc4)c3)nc(-c3ccc(-c4ccc5ccc6cccc7ccc4c5c67)cc3)n2)cc1]

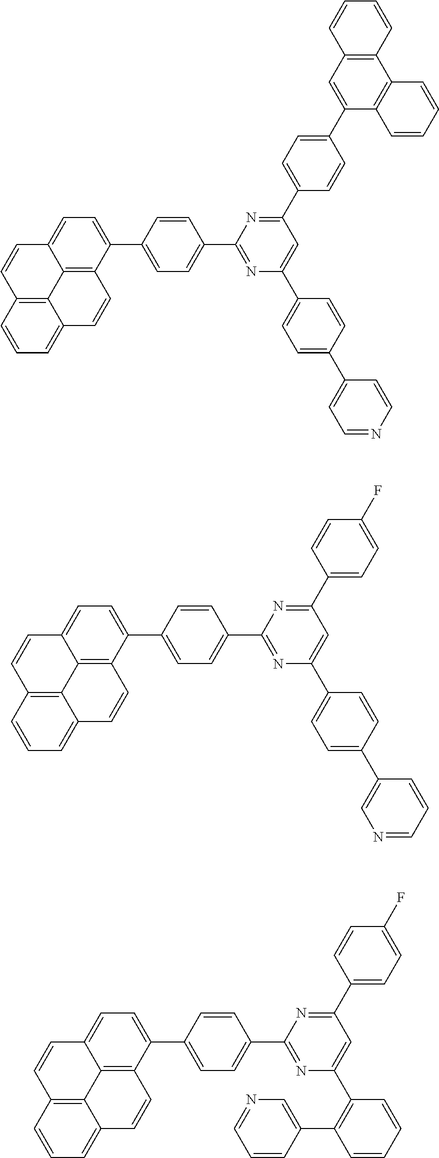 [SMILES: c1ccc2c(c1)cc(-c1ccc(-c3cc(-c4ccc(-c5ccncc5)cc4)nc(-c4ccc(-c5ccc6ccc7cccc8ccc5c6c78)cc4)n3)cc1)c1ccccc12 ; Fc1ccc(-c2cc(-c3ccc(-c4cccnc4)cc3)nc(-c3ccc(-c4ccc5ccc6cccc7ccc4c5c67)cc3)n2)cc1 ; Fc1ccc(-c2cc(-c3ccccc3-c3cccnc3)nc(-c3ccc(-c4ccc5ccc6cccc7ccc4c5c67)cc3)n2)cc1]

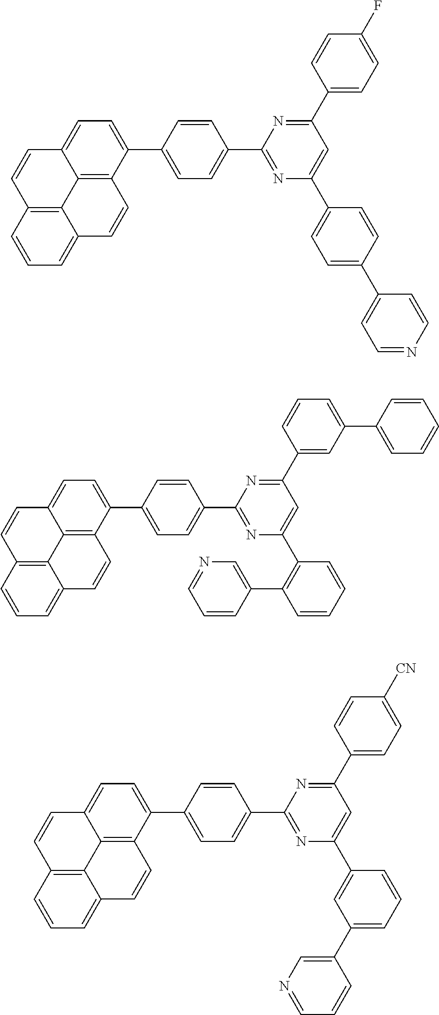 [SMILES: Fc1ccc(-c2cc(-c3ccc(-c4ccncc4)cc3)nc(-c3ccc(-c4ccc5ccc6cccc7ccc4c5c67)cc3)n2)cc1 ; c1ccc(-c2cccc(-c3cc(-c4ccccc4-c4cccnc4)nc(-c4ccc(-c5ccc6ccc7cccc8ccc5c6c78)cc4)n3)c2)cc1 ; N#Cc1ccc(-c2cc(-c3cccc(-c4cccnc4)c3)nc(-c3ccc(-c4ccc5ccc6cccc7ccc4c5c67)cc3)n2)cc1]

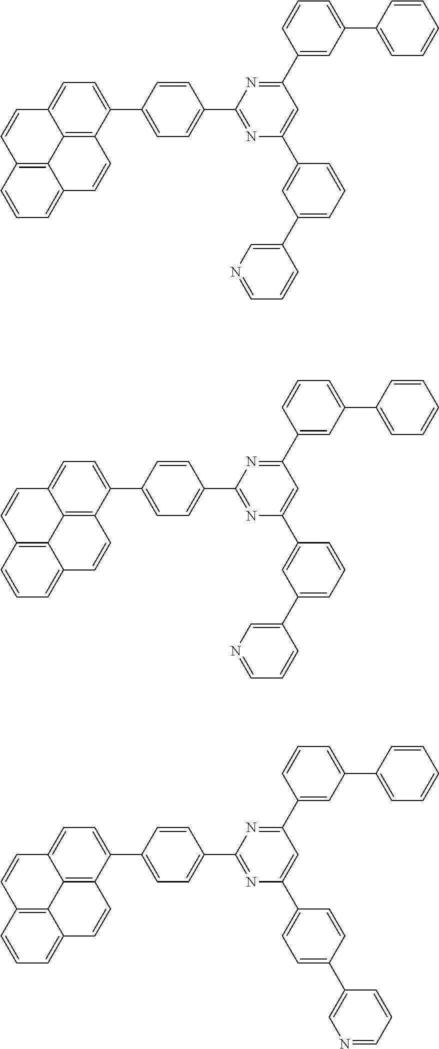 [SMILES: c1ccc(-c2cccc(-c3cc(-c4cccc(-c5cccnc5)c4)nc(-c4ccc(-c5ccc6ccc7cccc8ccc5c6c78)cc4)n3)c2)cc1 ; c1ccc(-c2cccc(-c3cc(-c4cccc(-c5cccnc5)c4)nc(-c4ccc(-c5ccc6ccc7cccc8ccc5c6c78)cc4)n3)c2)cc1 ; c1ccc(-c2cccc(-c3cc(-c4ccc(-c5cccnc5)cc4)nc(-c4ccc(-c5ccc6ccc7cccc8ccc5c6c78)cc4)n3)c2)cc1]

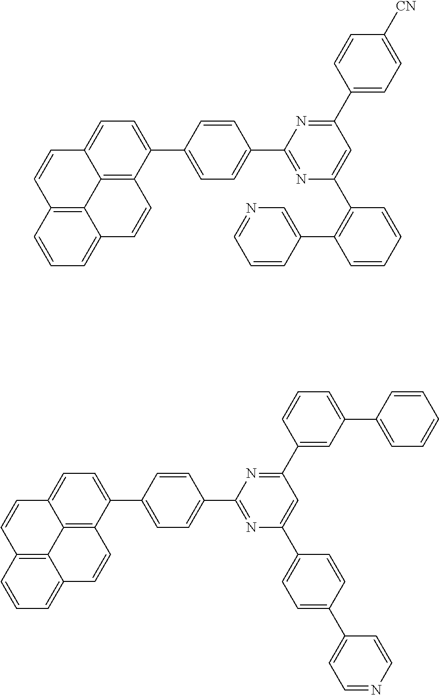 [SMILES: N#Cc1ccc(-c2cc(-c3ccccc3-c3cccnc3)nc(-c3ccc(-c4ccc5ccc6cccc7ccc4c5c67)cc3)n2)cc1 ; c1ccc(-c2cccc(-c3cc(-c4ccc(-c5ccncc5)cc4)nc(-c4ccc(-c5ccc6ccc7cccc8ccc5c6c78)cc4)n3)c2)cc1]

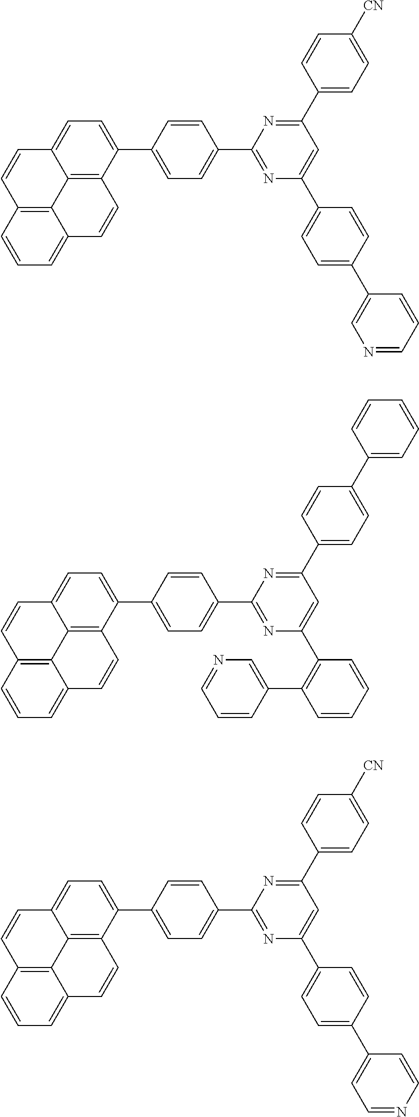 [SMILES: N#Cc1ccc(-c2cc(-c3ccc(-c4cccnc4)cc3)nc(-c3ccc(-c4ccc5ccc6cccc7ccc4c5c67)cc3)n2)cc1 ; c1ccc(-c2ccc(-c3cc(-c4ccccc4-c4cccnc4)nc(-c4ccc(-c5ccc6ccc7cccc8ccc5c6c78)cc4)n3)cc2)cc1 ; N#Cc1ccc(-c2cc(-c3ccc(-c4ccncc4)cc3)nc(-c3ccc(-c4ccc5ccc6cccc7ccc4c5c67)cc3)n2)cc1]

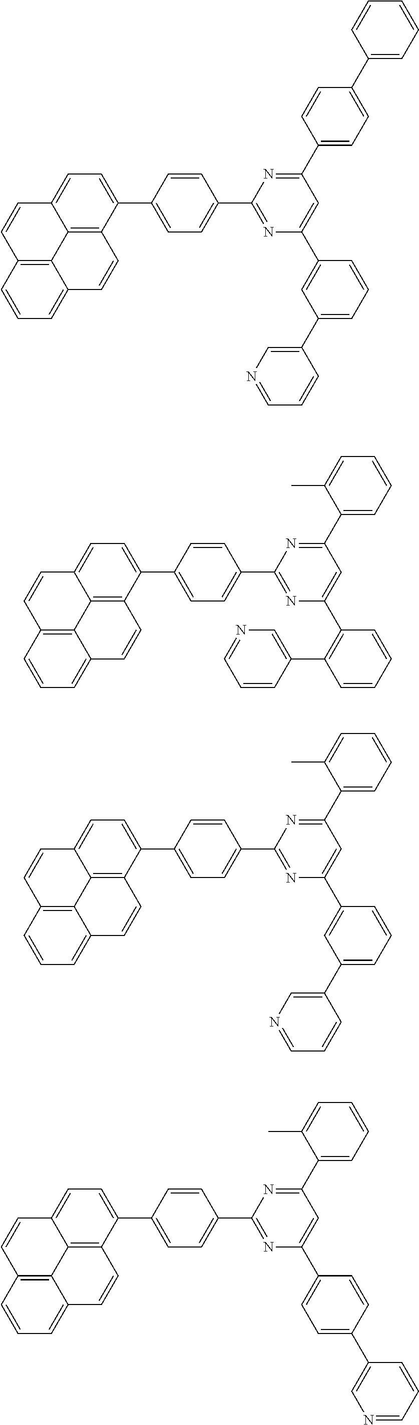

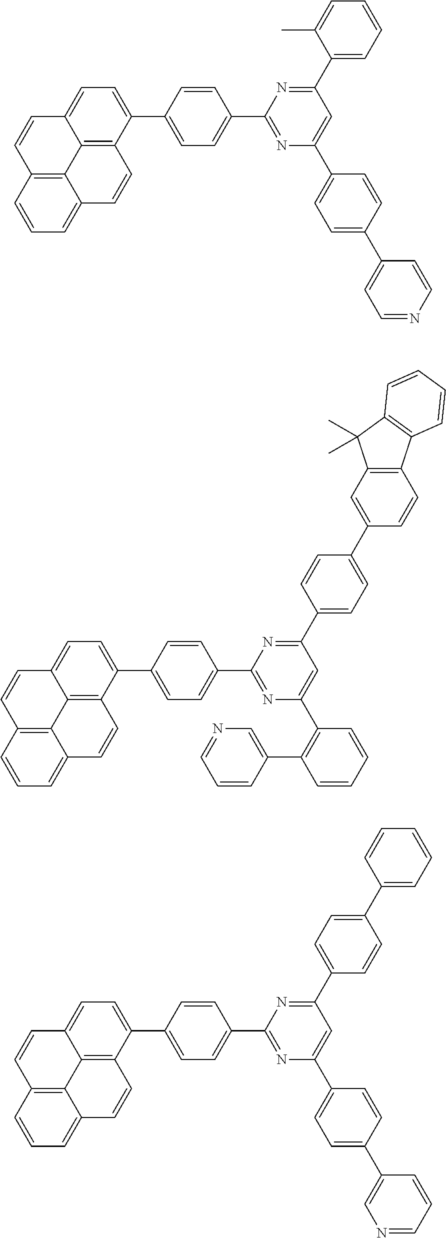 [SMILES: Cc1ccccc1-c1cc(-c2ccc(-c3ccncc3)cc2)nc(-c2ccc(-c3ccc4ccc5cccc6ccc3c4c56)cc2)n1 ; CC1(C)c2ccccc2-c2ccc(-c3ccc(-c4cc(-c5ccccc5-c5cccnc5)nc(-c5ccc(-c6ccc7ccc8cccc9ccc6c7c89)cc5)n4)cc3)cc21 ; c1ccc(-c2ccc(-c3cc(-c4ccc(-c5cccnc5)cc4)nc(-c4ccc(-c5ccc6ccc7cccc8ccc5c6c78)cc4)n3)cc2)cc1]

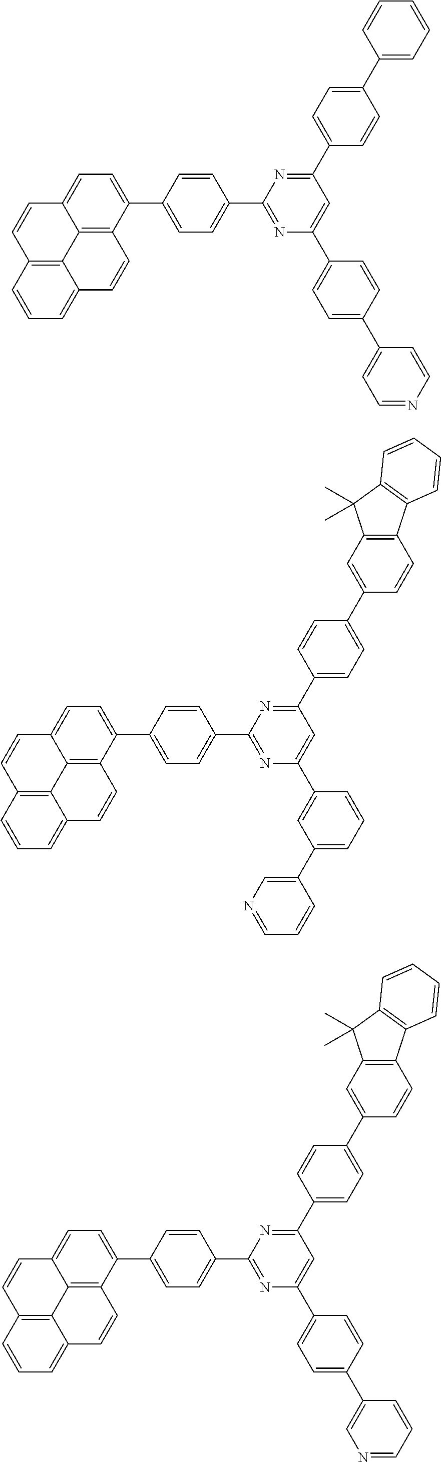

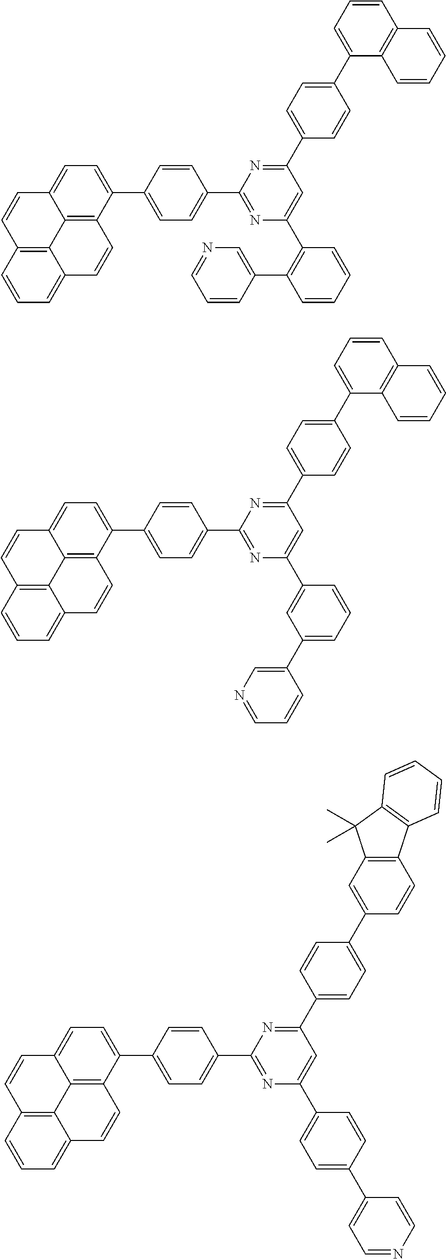

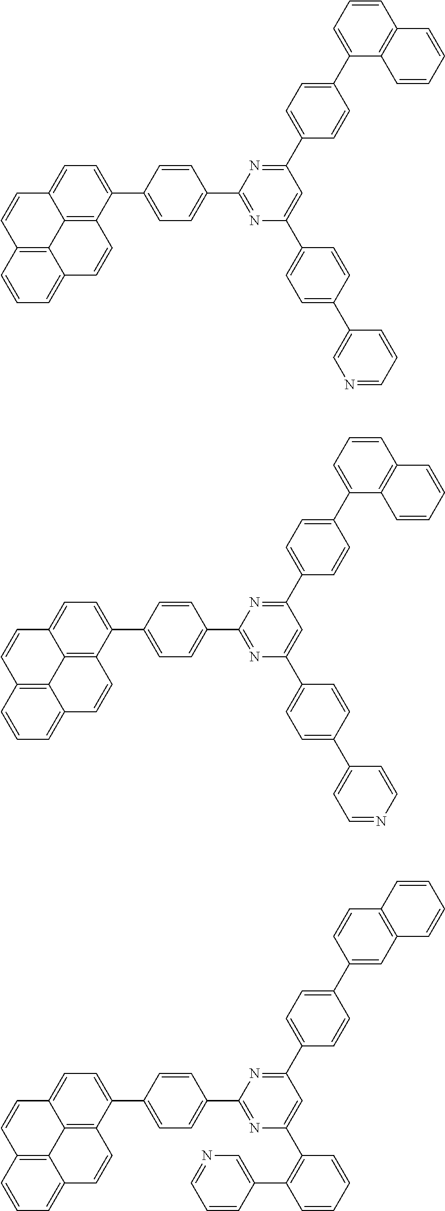 [SMILES: c1cncc(-c2ccc(-c3cc(-c4ccc(-c5cccc6ccccc56)cc4)nc(-c4ccc(-c5ccc6ccc7cccc8ccc5c6c78)cc4)n3)cc2)c1 ; c1ccc2c(-c3ccc(-c4cc(-c5ccc(-c6ccncc6)cc5)nc(-c5ccc(-c6ccc7ccc8cccc9ccc6c7c89)cc5)n4)cc3)cccc2c1 ; c1cncc(-c2ccccc2-c2cc(-c3ccc(-c4ccc5ccccc5c4)cc3)nc(-c3ccc(-c4ccc5ccc6cccc7ccc4c5c67)cc3)n2)c1]

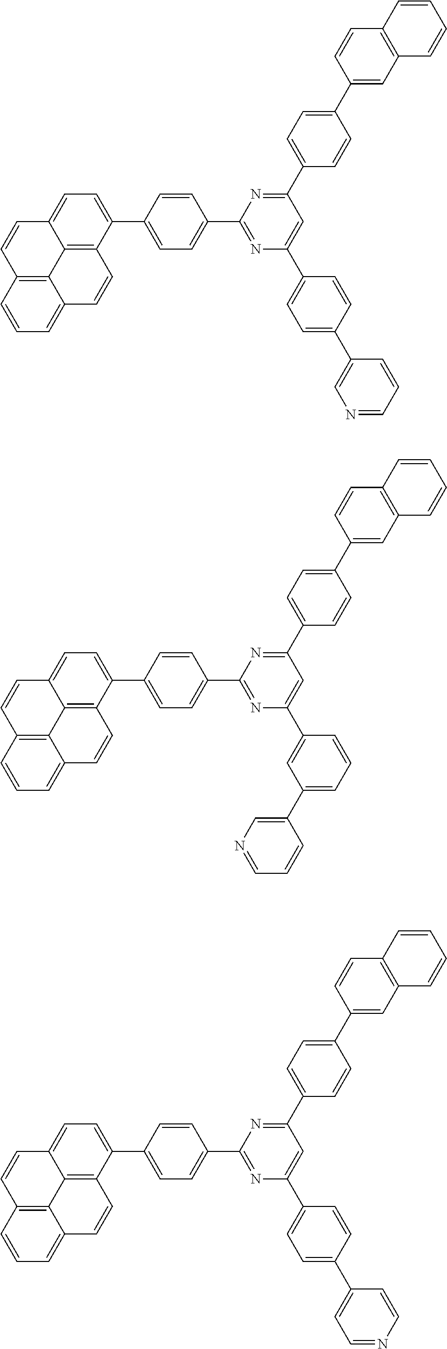 [SMILES: c1cncc(-c2ccc(-c3cc(-c4ccc(-c5ccc6ccccc6c5)cc4)nc(-c4ccc(-c5ccc6ccc7cccc8ccc5c6c78)cc4)n3)cc2)c1 ; c1cncc(-c2cccc(-c3cc(-c4ccc(-c5ccc6ccccc6c5)cc4)nc(-c4ccc(-c5ccc6ccc7cccc8ccc5c6c78)cc4)n3)c2)c1 ; c1ccc2cc(-c3ccc(-c4cc(-c5ccc(-c6ccncc6)cc5)nc(-c5ccc(-c6ccc7ccc8cccc9ccc6c7c89)cc5)n4)cc3)ccc2c1]

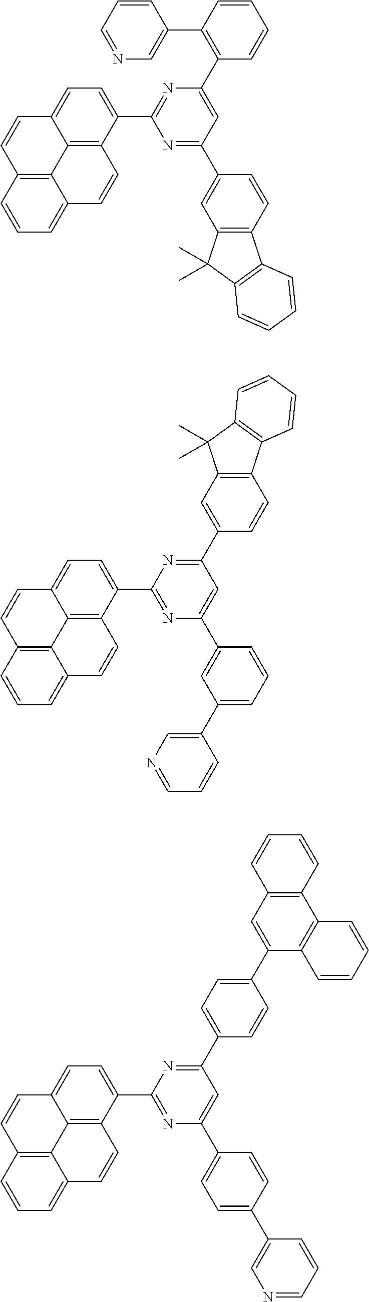

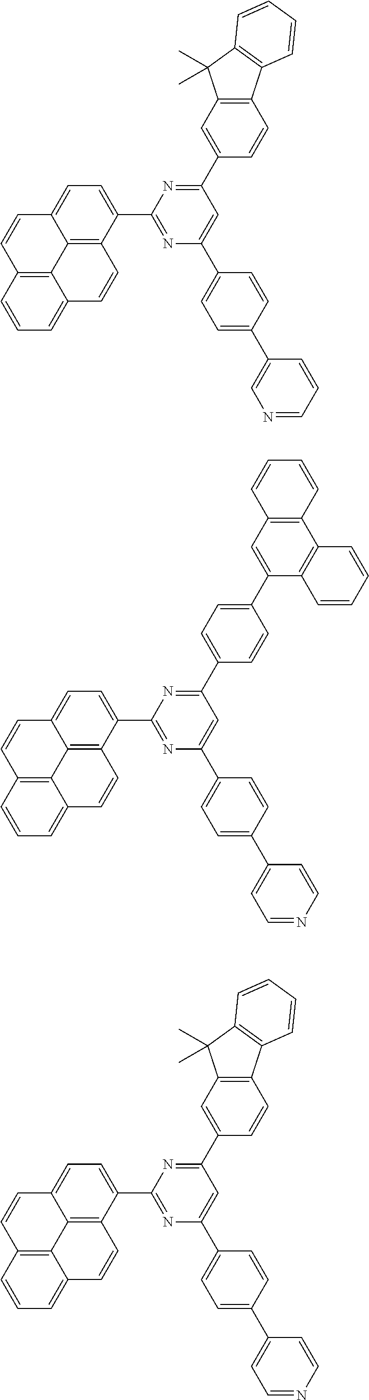

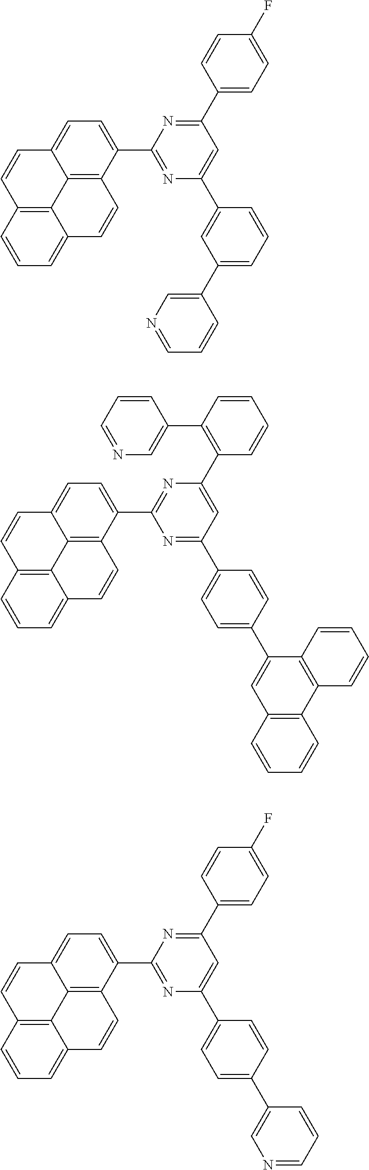

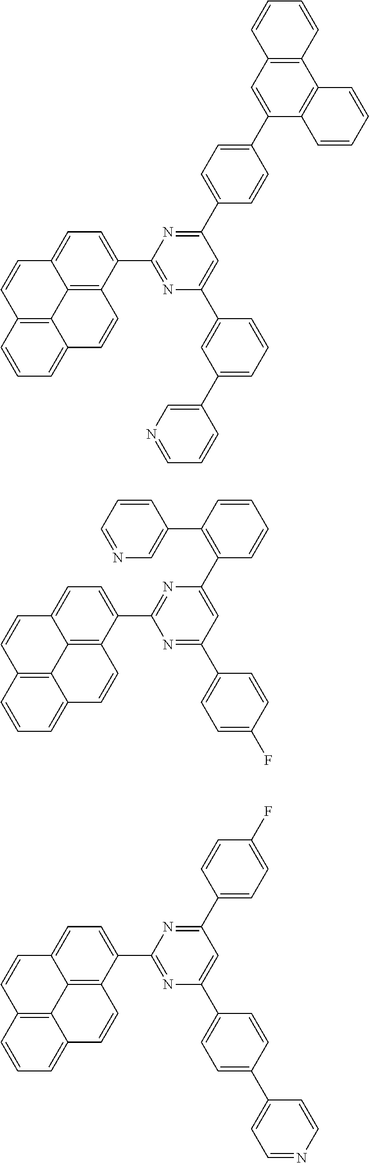

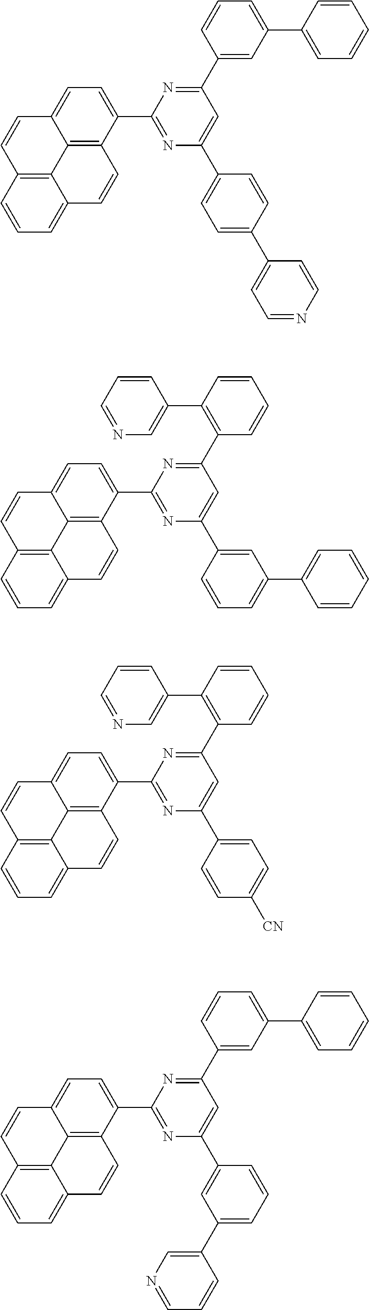 [SMILES: c1ccc(-c2cccc(-c3cc(-c4ccc(-c5ccncc5)cc4)nc(-c4ccc5ccc6cccc7ccc4c5c67)n3)c2)cc1 ; c1ccc(-c2cccc(-c3cc(-c4ccccc4-c4cccnc4)nc(-c4ccc5ccc6cccc7ccc4c5c67)n3)c2)cc1 ; N#Cc1ccc(-c2cc(-c3ccccc3-c3cccnc3)nc(-c3ccc4ccc5cccc6ccc3c4c56)n2)cc1 ; c1ccc(-c2cccc(-c3cc(-c4cccc(-c5cccnc5)c4)nc(-c4ccc5ccc6cccc7ccc4c5c67)n3)c2)cc1]

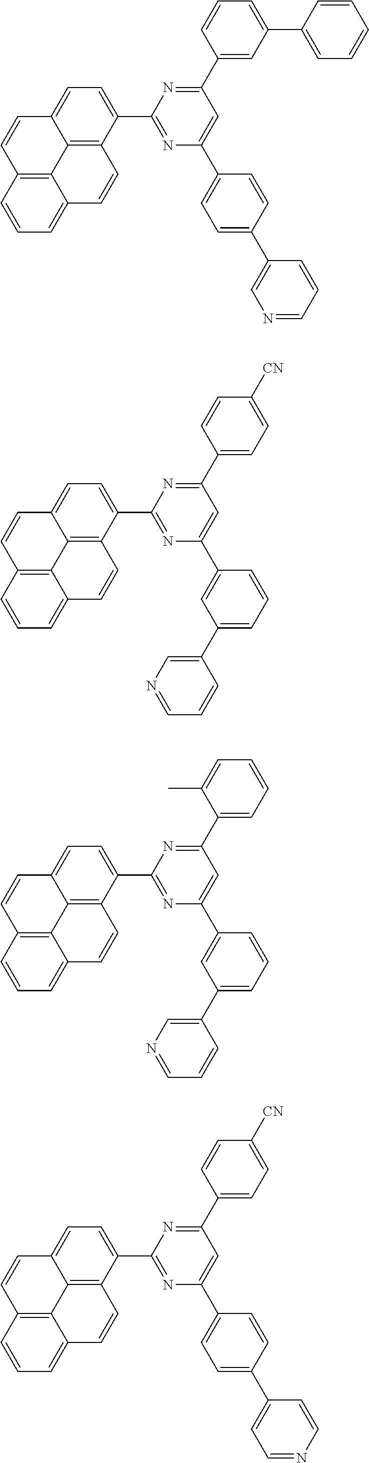 [SMILES: c1ccc(-c2cccc(-c3cc(-c4ccc(-c5cccnc5)cc4)nc(-c4ccc5ccc6cccc7ccc4c5c67)n3)c2)cc1 ; N#Cc1ccc(-c2cc(-c3cccc(-c4cccnc4)c3)nc(-c3ccc4ccc5cccc6ccc3c4c56)n2)cc1 ; Cc1ccccc1-c1cc(-c2cccc(-c3cccnc3)c2)nc(-c2ccc3ccc4cccc5ccc2c3c45)n1 ; N#Cc1ccc(-c2cc(-c3ccc(-c4ccncc4)cc3)nc(-c3ccc4ccc5cccc6ccc3c4c56)n2)cc1]

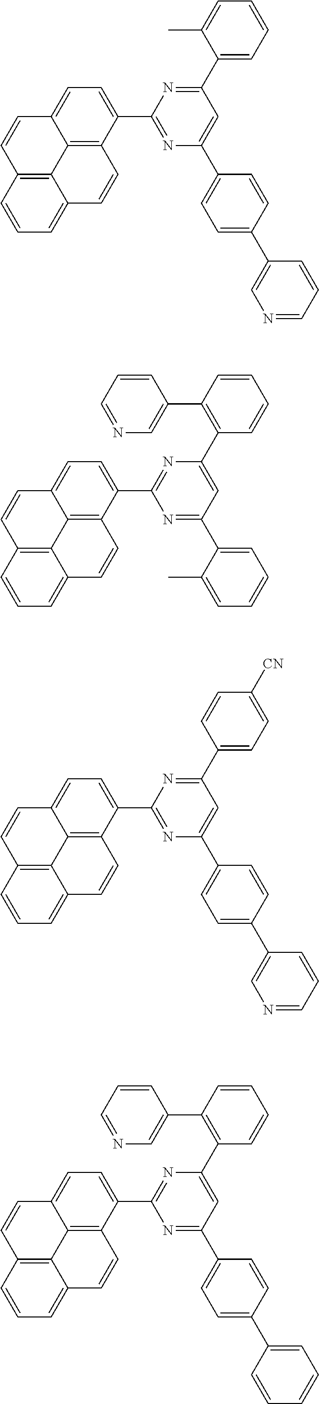 [SMILES: Cc1ccccc1-c1cc(-c2ccc(-c3cccnc3)cc2)nc(-c2ccc3ccc4cccc5ccc2c3c45)n1 ; Cc1ccccc1-c1cc(-c2ccccc2-c2cccnc2)nc(-c2ccc3ccc4cccc5ccc2c3c45)n1 ; N#Cc1ccc(-c2cc(-c3ccc(-c4cccnc4)cc3)nc(-c3ccc4ccc5cccc6ccc3c4c56)n2)cc1 ; c1ccc(-c2ccc(-c3cc(-c4ccccc4-c4cccnc4)nc(-c4ccc5ccc6cccc7ccc4c5c67)n3)cc2)cc1]

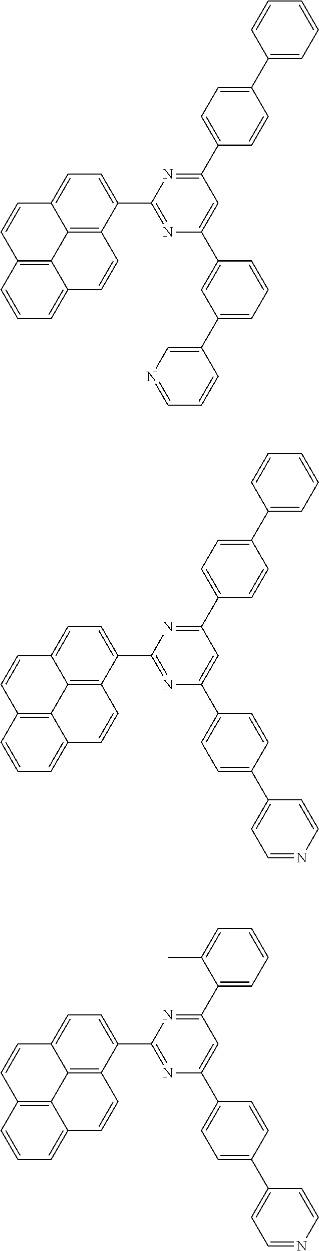 [SMILES: c1ccc(-c2ccc(-c3cc(-c4cccc(-c5cccnc5)c4)nc(-c4ccc5ccc6cccc7ccc4c5c67)n3)cc2)cc1 ; c1ccc(-c2ccc(-c3cc(-c4ccc(-c5ccncc5)cc4)nc(-c4ccc5ccc6cccc7ccc4c5c67)n3)cc2)cc1 ; Cc1ccccc1-c1cc(-c2ccc(-c3ccncc3)cc2)nc(-c2ccc3ccc4cccc5ccc2c3c45)n1]

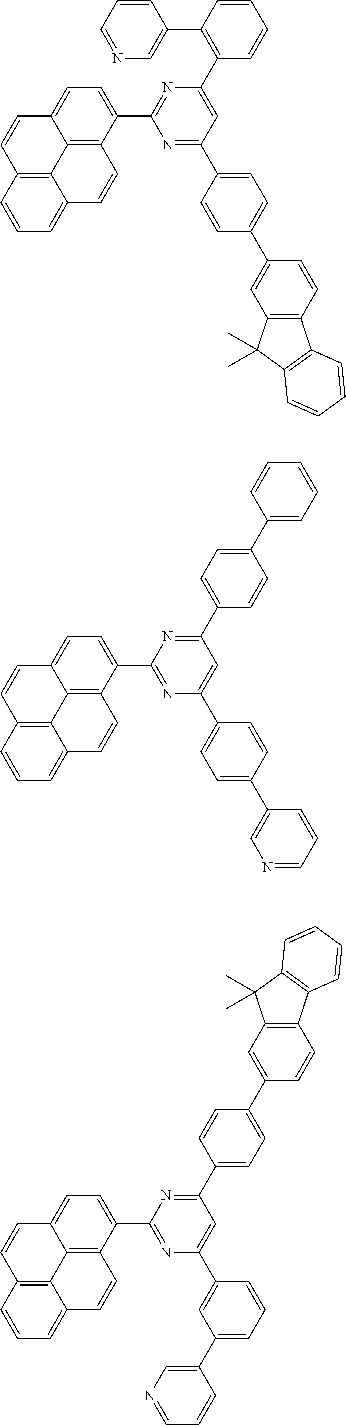 [SMILES: CC1(C)c2ccccc2-c2ccc(-c3ccc(-c4cc(-c5ccccc5-c5cccnc5)nc(-c5ccc6ccc7cccc8ccc5c6c78)n4)cc3)cc21 ; c1ccc(-c2ccc(-c3cc(-c4ccc(-c5cccnc5)cc4)nc(-c4ccc5ccc6cccc7ccc4c5c67)n3)cc2)cc1 ; CC1(C)c2ccccc2-c2ccc(-c3ccc(-c4cc(-c5cccc(-c6cccnc6)c5)nc(-c5ccc6ccc7cccc8ccc5c6c78)n4)cc3)cc21]

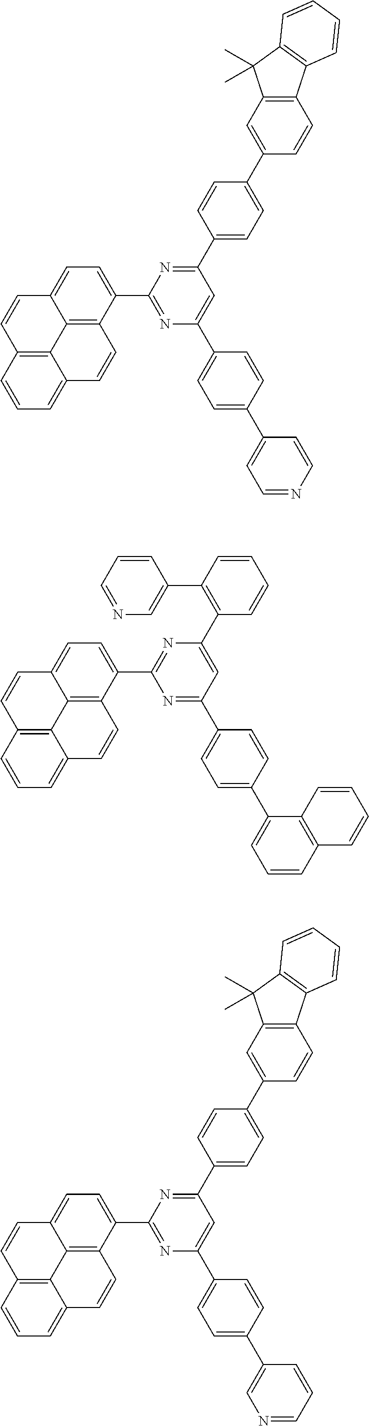 [SMILES: CC1(C)c2ccccc2-c2ccc(-c3ccc(-c4cc(-c5ccc(-c6ccncc6)cc5)nc(-c5ccc6ccc7cccc8ccc5c6c78)n4)cc3)cc21 ; c1cncc(-c2ccccc2-c2cc(-c3ccc(-c4cccc5ccccc45)cc3)nc(-c3ccc4ccc5cccc6ccc3c4c56)n2)c1 ; CC1(C)c2ccccc2-c2ccc(-c3ccc(-c4cc(-c5ccc(-c6cccnc6)cc5)nc(-c5ccc6ccc7cccc8ccc5c6c78)n4)cc3)cc21]

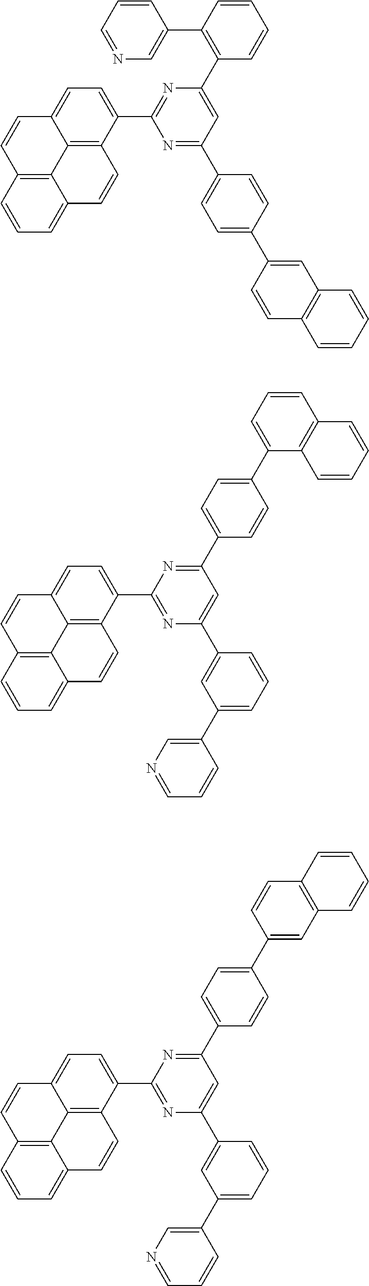 [SMILES: c1cncc(-c2ccccc2-c2cc(-c3ccc(-c4ccc5ccccc5c4)cc3)nc(-c3ccc4ccc5cccc6ccc3c4c56)n2)c1 ; c1cncc(-c2cccc(-c3cc(-c4ccc(-c5cccc6ccccc56)cc4)nc(-c4ccc5ccc6cccc7ccc4c5c67)n3)c2)c1 ; c1cncc(-c2cccc(-c3cc(-c4ccc(-c5ccc6ccccc6c5)cc4)nc(-c4ccc5ccc6cccc7ccc4c5c67)n3)c2)c1]

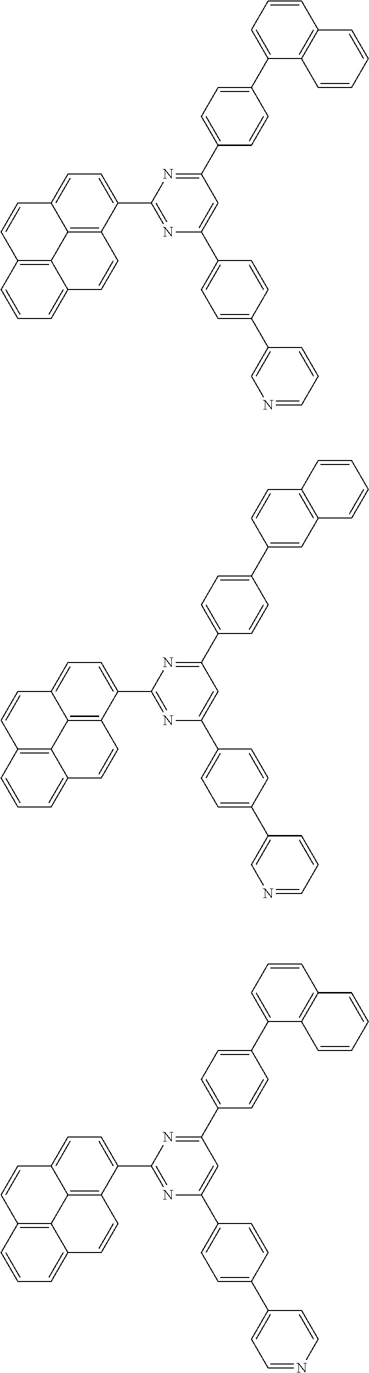 [SMILES: c1cncc(-c2ccc(-c3cc(-c4ccc(-c5cccc6ccccc56)cc4)nc(-c4ccc5ccc6cccc7ccc4c5c67)n3)cc2)c1 ; c1cncc(-c2ccc(-c3cc(-c4ccc(-c5ccc6ccccc6c5)cc4)nc(-c4ccc5ccc6cccc7ccc4c5c67)n3)cc2)c1 ; c1ccc2c(-c3ccc(-c4cc(-c5ccc(-c6ccncc6)cc5)nc(-c5ccc6ccc7cccc8ccc5c6c78)n4)cc3)cccc2c1]

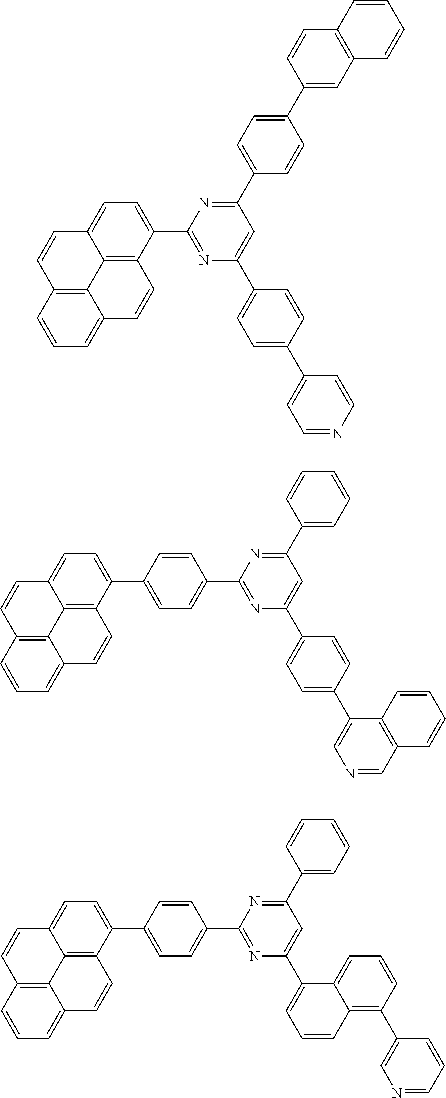

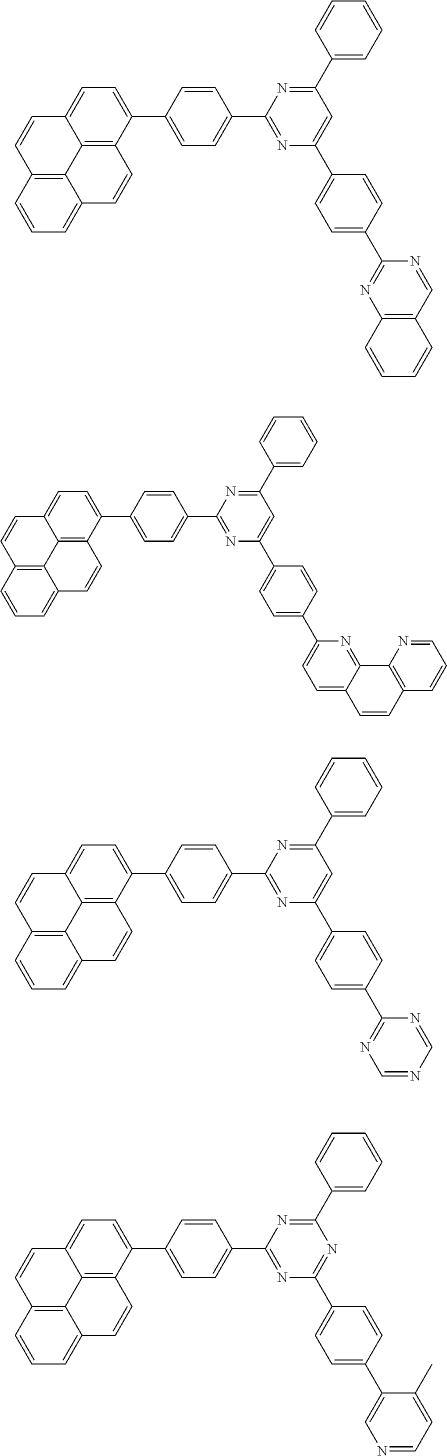

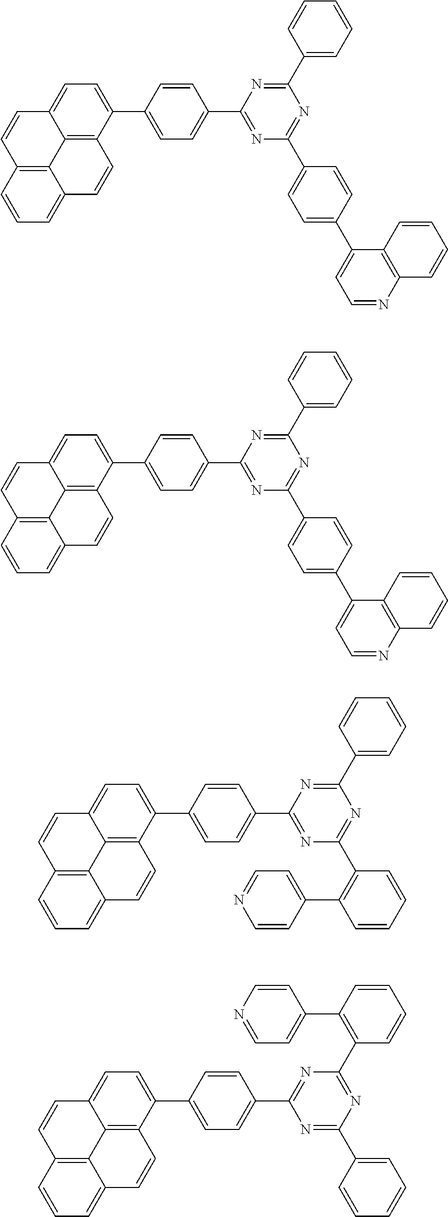

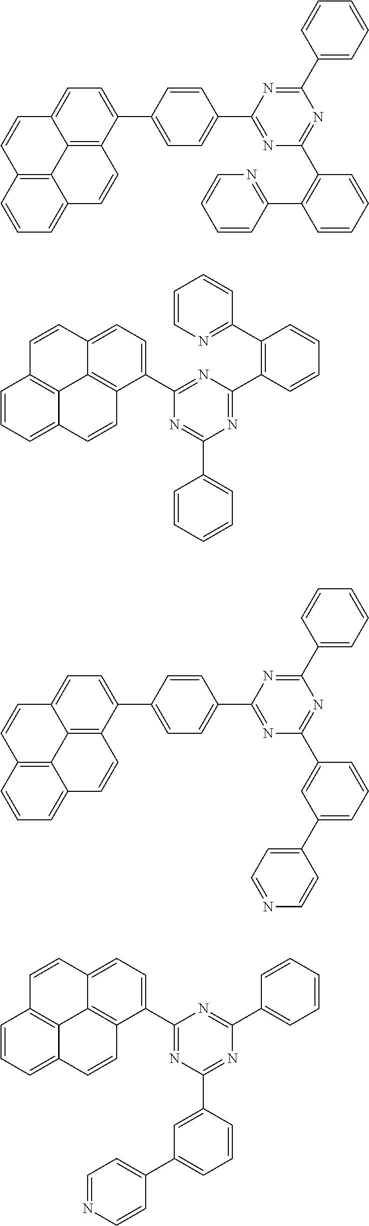

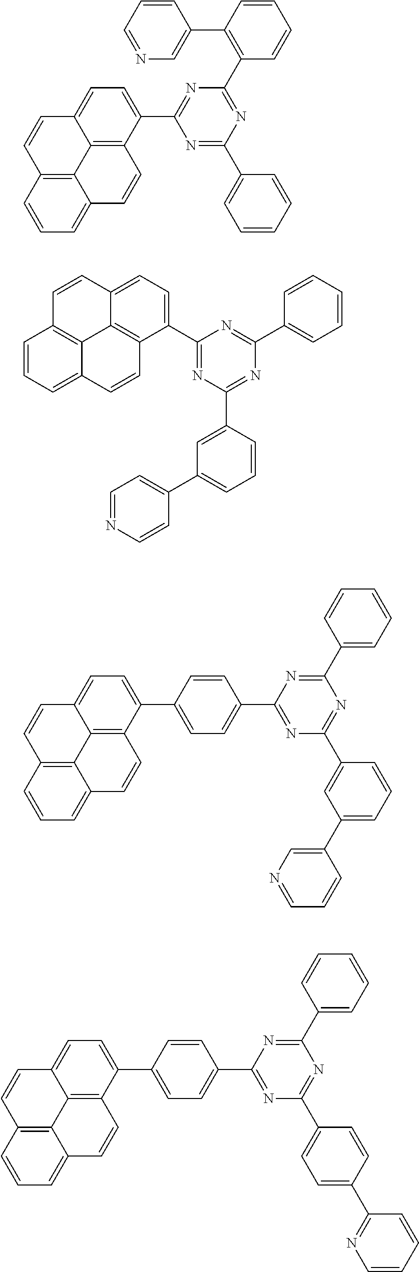

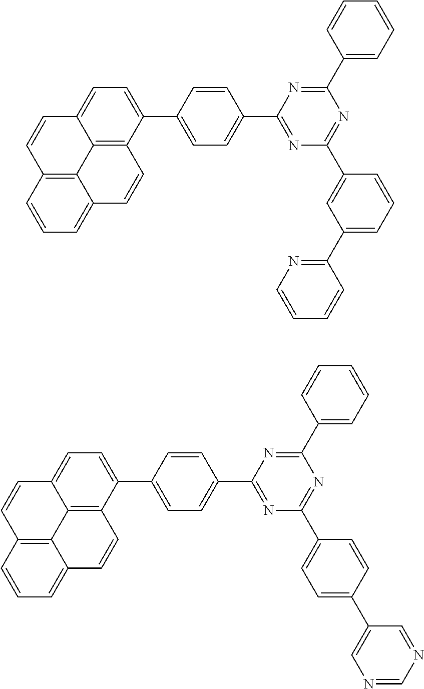 [SMILES: c1ccc(-c2nc(-c3ccc(-c4ccc5ccc6cccc7ccc4c5c67)cc3)nc(-c3cccc(-c4ccccn4)c3)n2)cc1 ; c1ccc(-c2nc(-c3ccc(-c4cncnc4)cc3)nc(-c3ccc(-c4ccc5ccc6cccc7ccc4c5c67)cc3)n2)cc1]

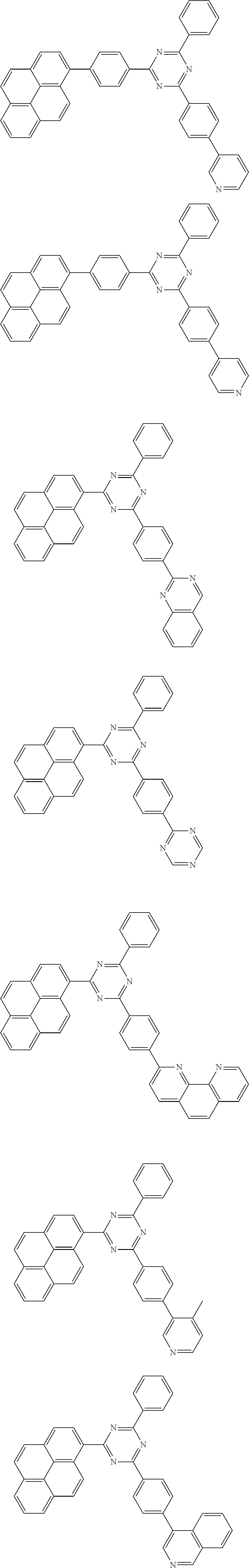

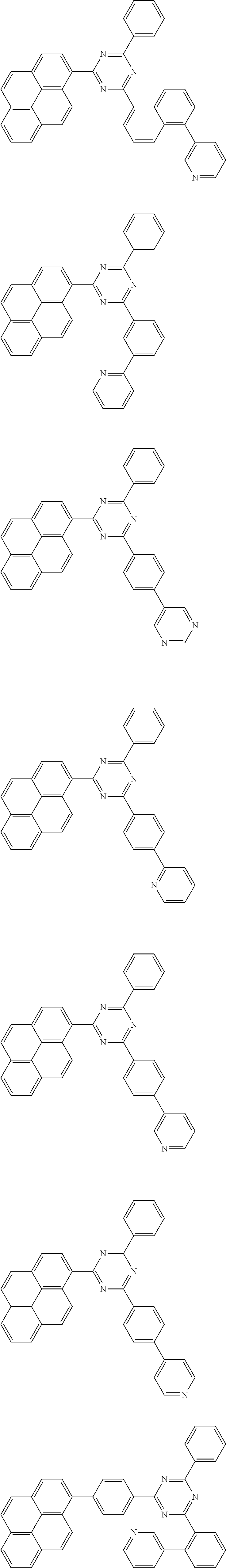

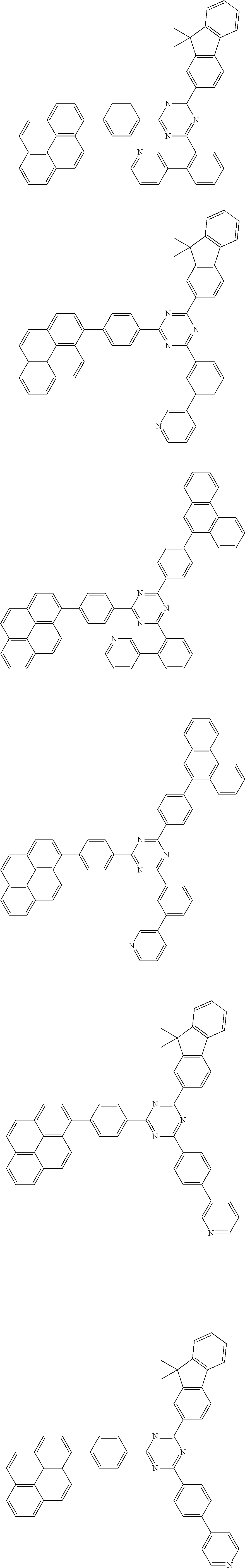

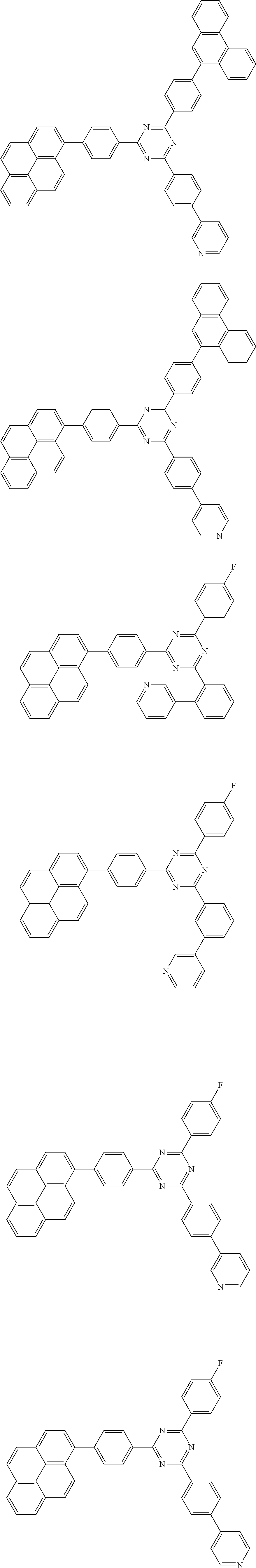

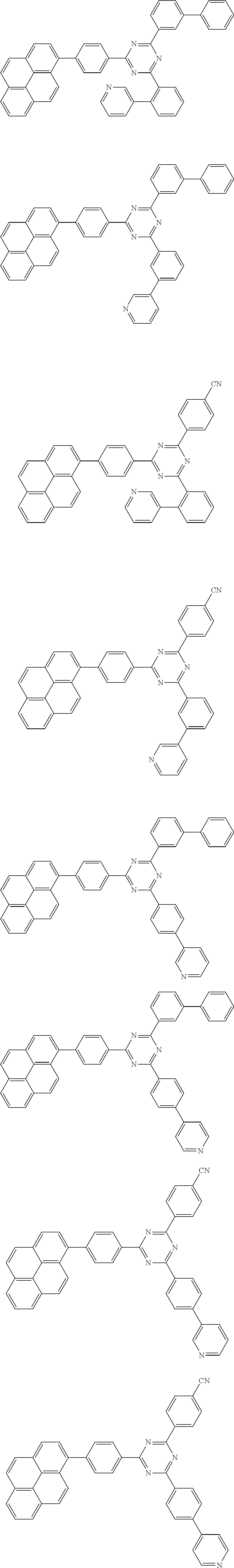

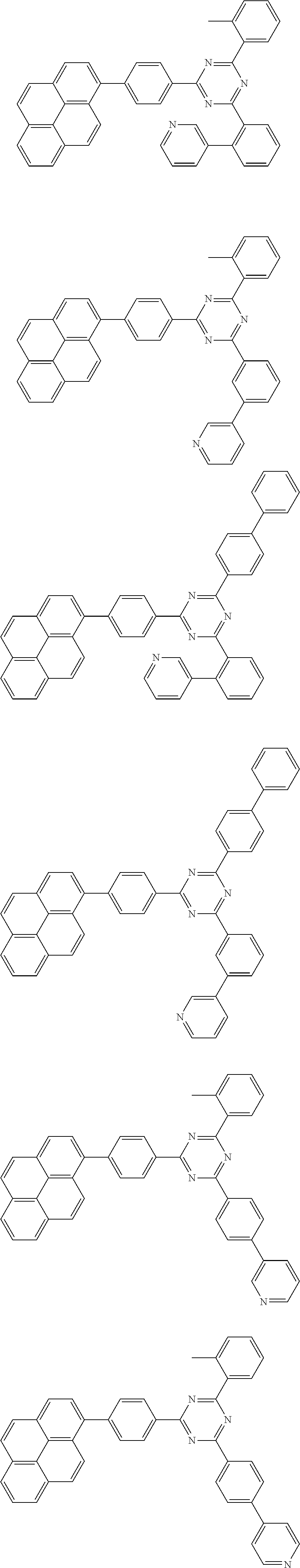

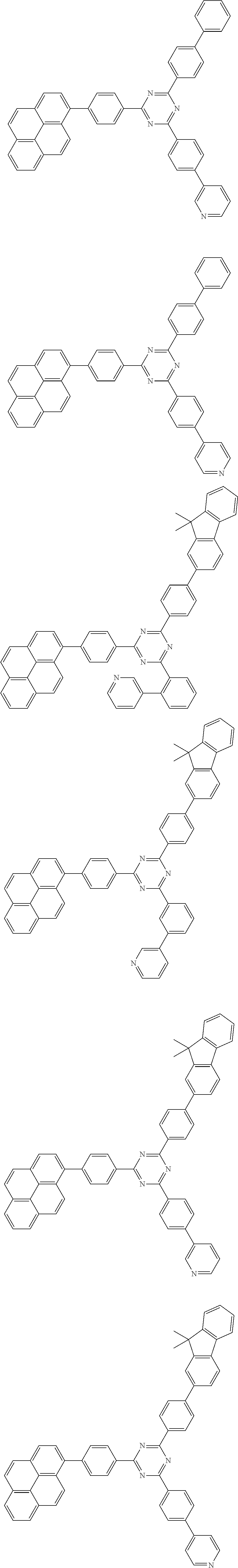

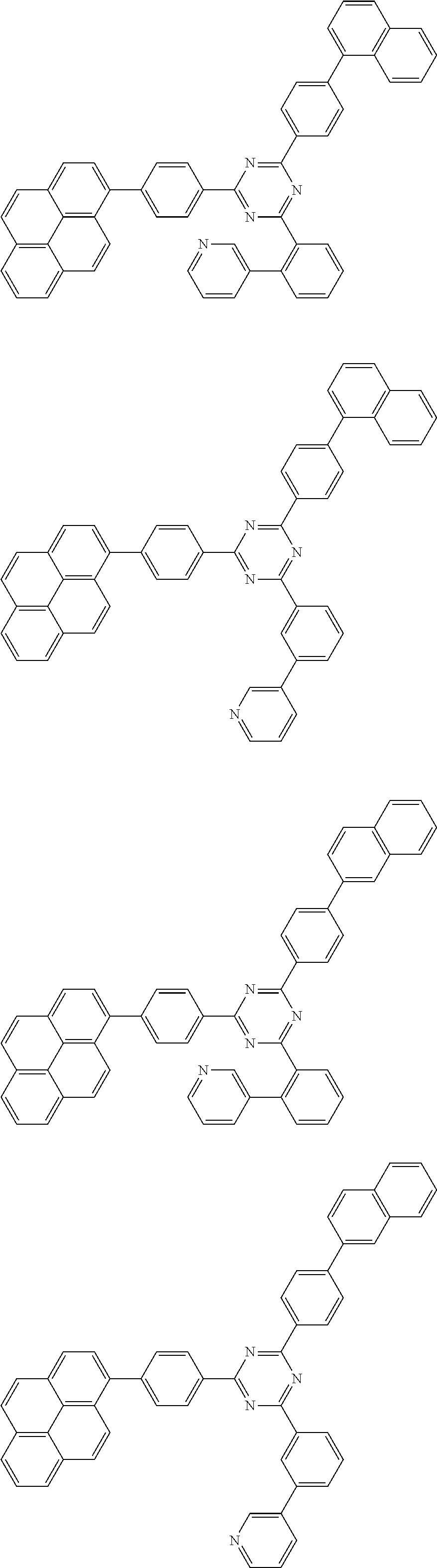 [SMILES: c1cncc(-c2ccccc2-c2nc(-c3ccc(-c4cccc5ccccc45)cc3)nc(-c3ccc(-c4ccc5ccc6cccc7ccc4c5c67)cc3)n2)c1 ; c1cncc(-c2cccc(-c3nc(-c4ccc(-c5cccc6ccccc56)cc4)nc(-c4ccc(-c5ccc6ccc7cccc8ccc5c6c78)cc4)n3)c2)c1 ; c1cncc(-c2ccccc2-c2nc(-c3ccc(-c4ccc5ccccc5c4)cc3)nc(-c3ccc(-c4ccc5ccc6cccc7ccc4c5c67)cc3)n2)c1 ; c1cncc(-c2cccc(-c3nc(-c4ccc(-c5ccc6ccccc6c5)cc4)nc(-c4ccc(-c5ccc6ccc7cccc8ccc5c6c78)cc4)n3)c2)c1]

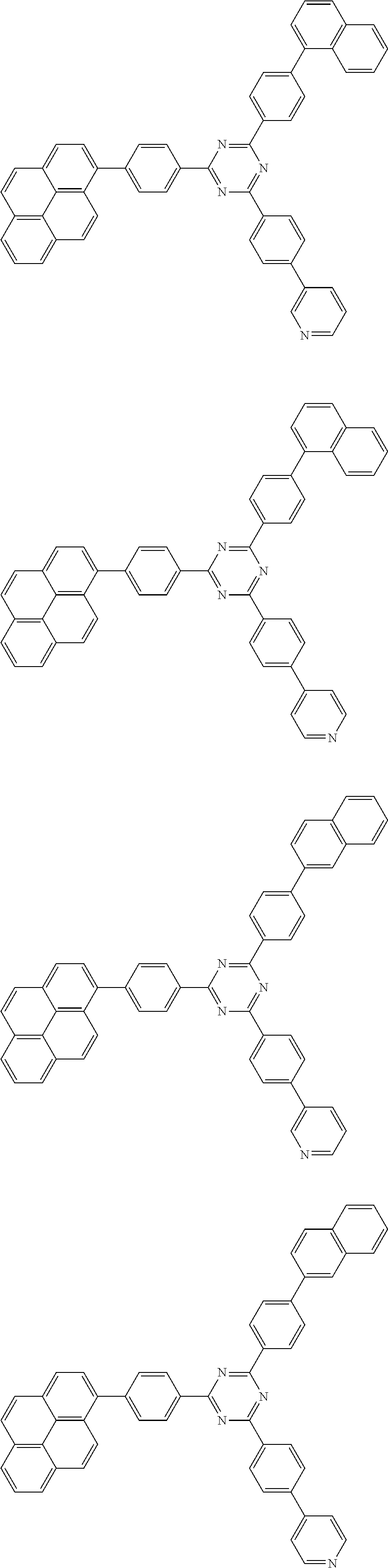 [SMILES: c1cncc(-c2ccc(-c3nc(-c4ccc(-c5cccc6ccccc56)cc4)nc(-c4ccc(-c5ccc6ccc7cccc8ccc5c6c78)cc4)n3)cc2)c1 ; c1ccc2c(-c3ccc(-c4nc(-c5ccc(-c6ccncc6)cc5)nc(-c5ccc(-c6ccc7ccc8cccc9ccc6c7c89)cc5)n4)cc3)cccc2c1 ; c1cncc(-c2ccc(-c3nc(-c4ccc(-c5ccc6ccccc6c5)cc4)nc(-c4ccc(-c5ccc6ccc7cccc8ccc5c6c78)cc4)n3)cc2)c1 ; c1ccc2cc(-c3ccc(-c4nc(-c5ccc(-c6ccncc6)cc5)nc(-c5ccc(-c6ccc7ccc8cccc9ccc6c7c89)cc5)n4)cc3)ccc2c1]

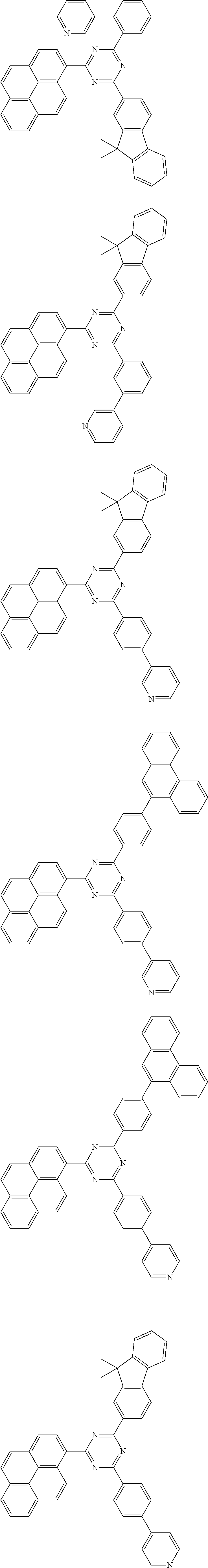

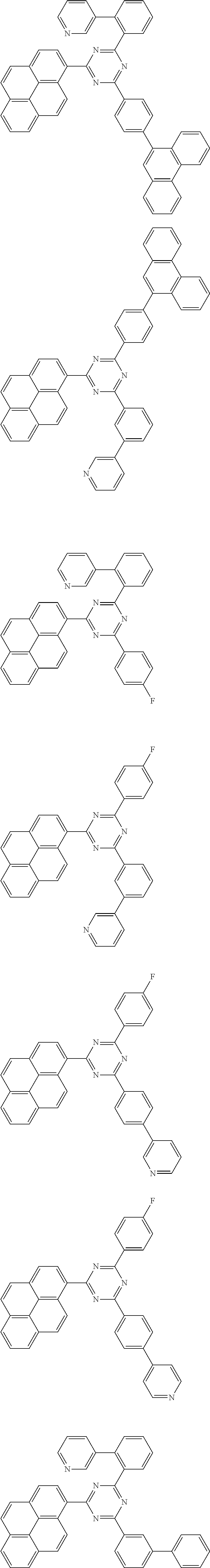

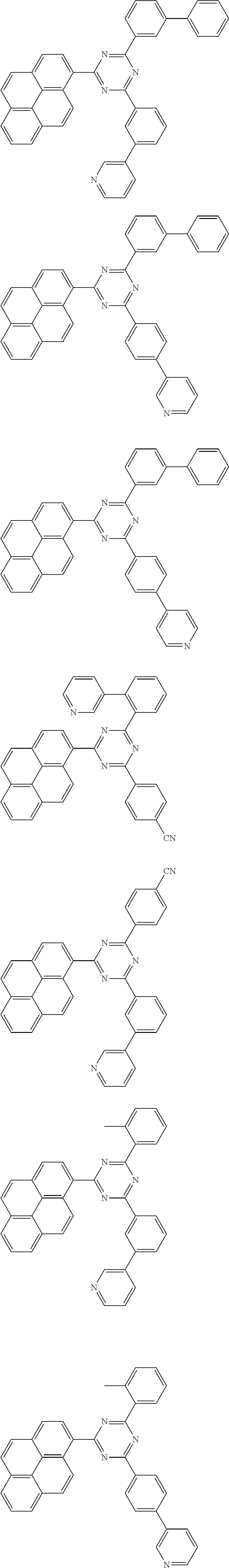

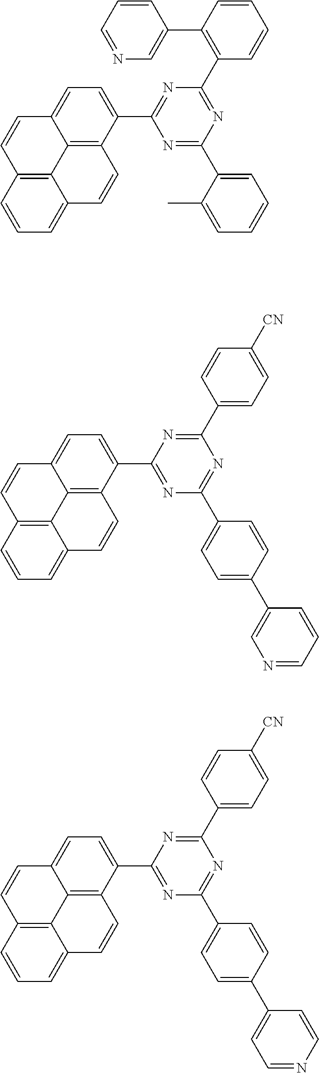 [SMILES: Cc1ccccc1-c1nc(-c2ccccc2-c2cccnc2)nc(-c2ccc3ccc4cccc5ccc2c3c45)n1 ; N#Cc1ccc(-c2nc(-c3ccc(-c4cccnc4)cc3)nc(-c3ccc4ccc5cccc6ccc3c4c56)n2)cc1 ; N#Cc1ccc(-c2nc(-c3ccc(-c4ccncc4)cc3)nc(-c3ccc4ccc5cccc6ccc3c4c56)n2)cc1]

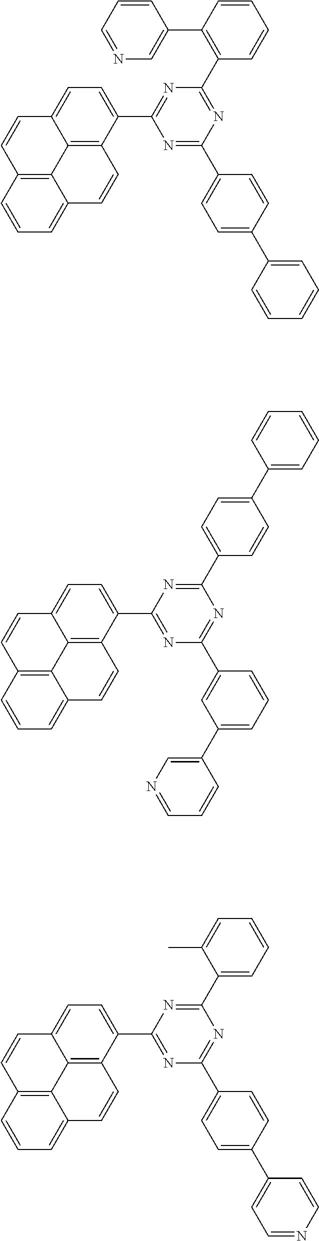 [SMILES: c1ccc(-c2ccc(-c3nc(-c4ccccc4-c4cccnc4)nc(-c4ccc5ccc6cccc7ccc4c5c67)n3)cc2)cc1 ; c1ccc(-c2ccc(-c3nc(-c4cccc(-c5cccnc5)c4)nc(-c4ccc5ccc6cccc7ccc4c5c67)n3)cc2)cc1 ; Cc1ccccc1-c1nc(-c2ccc(-c3ccncc3)cc2)nc(-c2ccc3ccc4cccc5ccc2c3c45)n1]

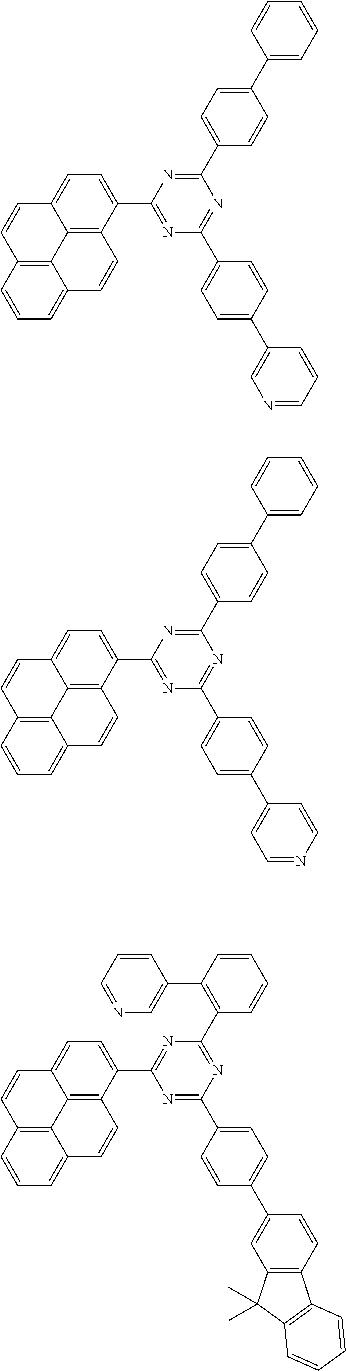 [SMILES: c1ccc(-c2ccc(-c3nc(-c4ccc(-c5cccnc5)cc4)nc(-c4ccc5ccc6cccc7ccc4c5c67)n3)cc2)cc1 ; c1ccc(-c2ccc(-c3nc(-c4ccc(-c5ccncc5)cc4)nc(-c4ccc5ccc6cccc7ccc4c5c67)n3)cc2)cc1 ; CC1(C)c2ccccc2-c2ccc(-c3ccc(-c4nc(-c5ccccc5-c5cccnc5)nc(-c5ccc6ccc7cccc8ccc5c6c78)n4)cc3)cc21]

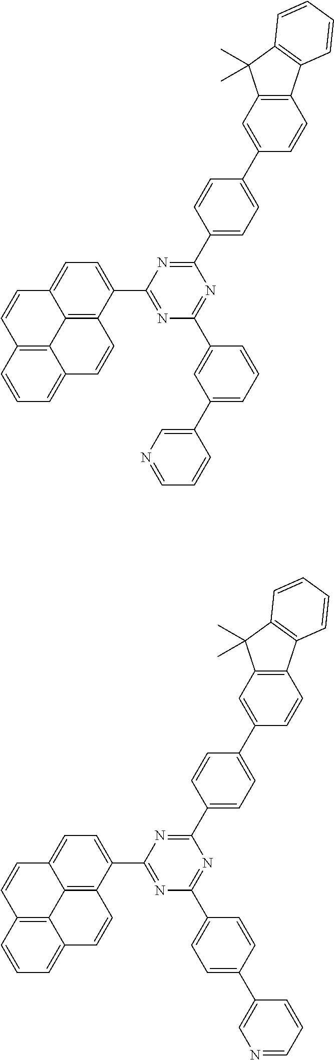 [SMILES: CC1(C)c2ccccc2-c2ccc(-c3ccc(-c4nc(-c5cccc(-c6cccnc6)c5)nc(-c5ccc6ccc7cccc8ccc5c6c78)n4)cc3)cc21 ; CC1(C)c2ccccc2-c2ccc(-c3ccc(-c4nc(-c5ccc(-c6cccnc6)cc5)nc(-c5ccc6ccc7cccc8ccc5c6c78)n4)cc3)cc21]

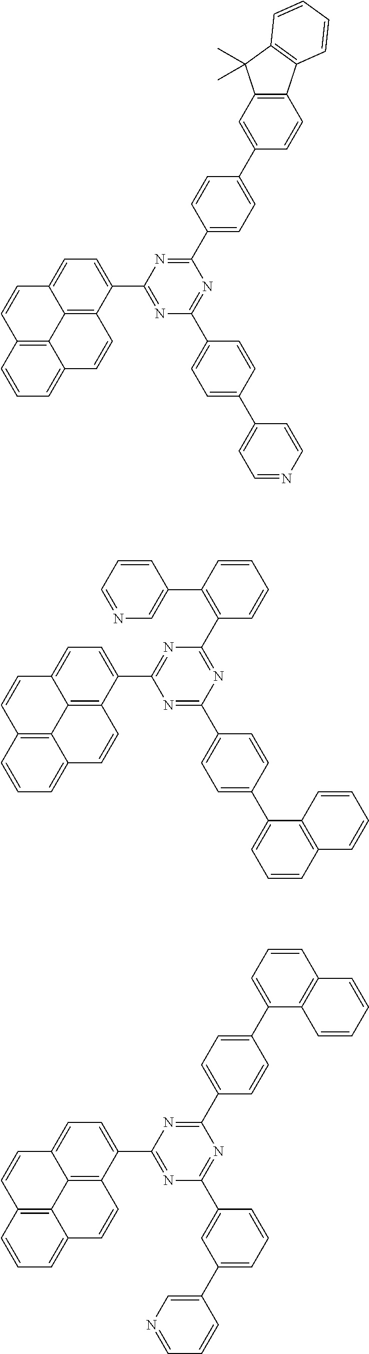 [SMILES: CC1(C)c2ccccc2-c2ccc(-c3ccc(-c4nc(-c5ccc(-c6ccncc6)cc5)nc(-c5ccc6ccc7cccc8ccc5c6c78)n4)cc3)cc21 ; c1cncc(-c2ccccc2-c2nc(-c3ccc(-c4cccc5ccccc45)cc3)nc(-c3ccc4ccc5cccc6ccc3c4c56)n2)c1 ; c1cncc(-c2cccc(-c3nc(-c4ccc(-c5cccc6ccccc56)cc4)nc(-c4ccc5ccc6cccc7ccc4c5c67)n3)c2)c1]

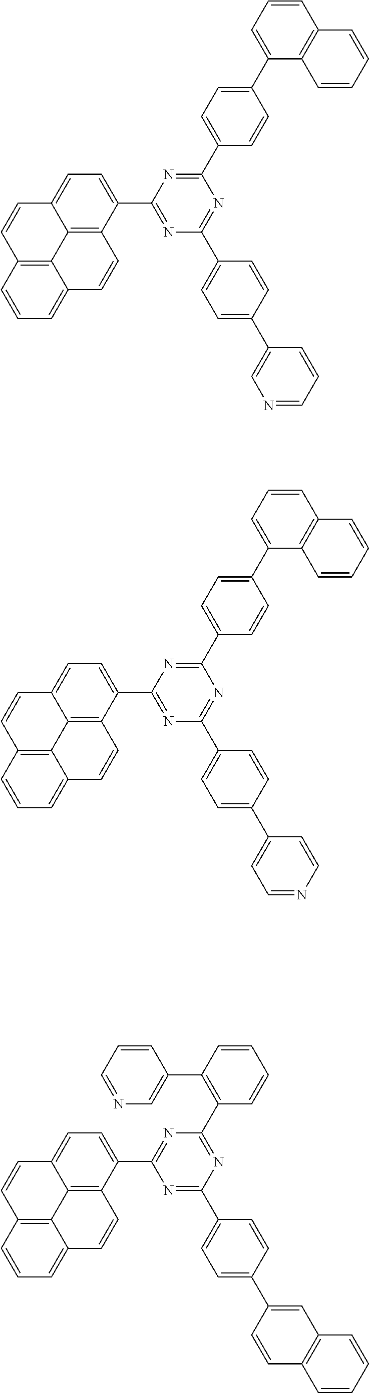 [SMILES: c1cncc(-c2ccc(-c3nc(-c4ccc(-c5cccc6ccccc56)cc4)nc(-c4ccc5ccc6cccc7ccc4c5c67)n3)cc2)c1 ; c1ccc2c(-c3ccc(-c4nc(-c5ccc(-c6ccncc6)cc5)nc(-c5ccc6ccc7cccc8ccc5c6c78)n4)cc3)cccc2c1 ; c1cncc(-c2ccccc2-c2nc(-c3ccc(-c4ccc5ccccc5c4)cc3)nc(-c3ccc4ccc5cccc6ccc3c4c56)n2)c1]

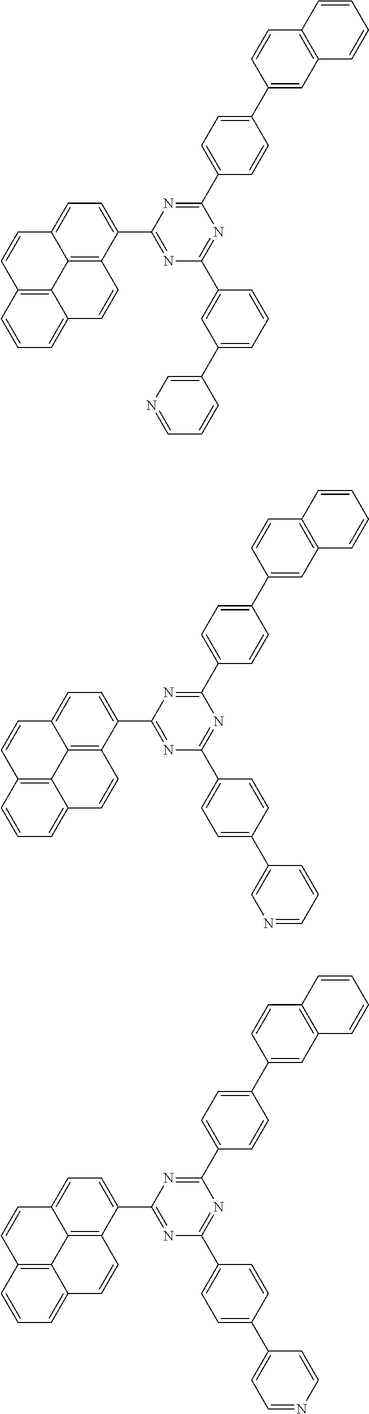 [SMILES: c1cncc(-c2cccc(-c3nc(-c4ccc(-c5ccc6ccccc6c5)cc4)nc(-c4ccc5ccc6cccc7ccc4c5c67)n3)c2)c1 ; c1cncc(-c2ccc(-c3nc(-c4ccc(-c5ccc6ccccc6c5)cc4)nc(-c4ccc5ccc6cccc7ccc4c5c67)n3)cc2)c1 ; c1ccc2cc(-c3ccc(-c4nc(-c5ccc(-c6ccncc6)cc5)nc(-c5ccc6ccc7cccc8ccc5c6c78)n4)cc3)ccc2c1]

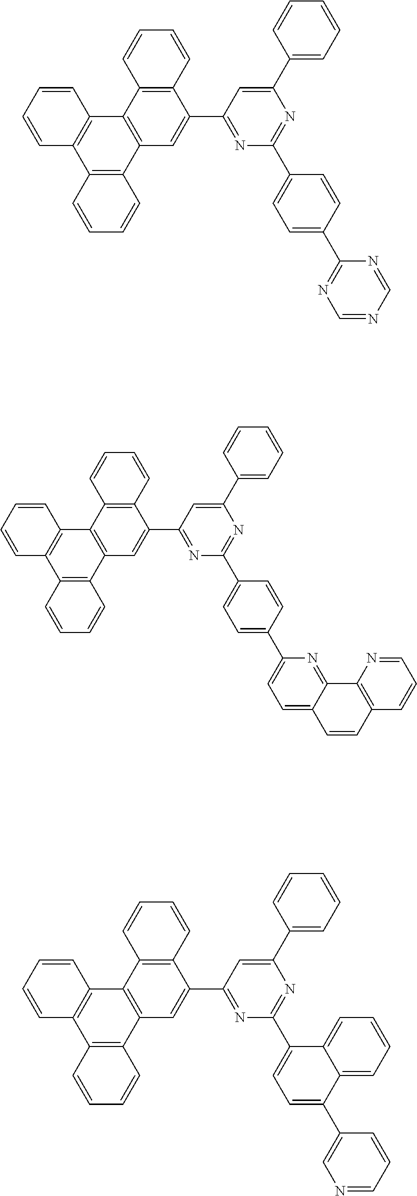 [SMILES: c1ccc(-c2cc(-c3cc4c5ccccc5c5ccccc5c4c4ccccc34)nc(-c3ccc(-c4ncncn4)cc3)n2)cc1 ; c1ccc(-c2cc(-c3cc4c5ccccc5c5ccccc5c4c4ccccc34)nc(-c3ccc(-c4ccc5ccc6cccnc6c5n4)cc3)n2)cc1 ; c1ccc(-c2cc(-c3cc4c5ccccc5c5ccccc5c4c4ccccc34)nc(-c3ccc(-c4cccnc4)c4ccccc34)n2)cc1]

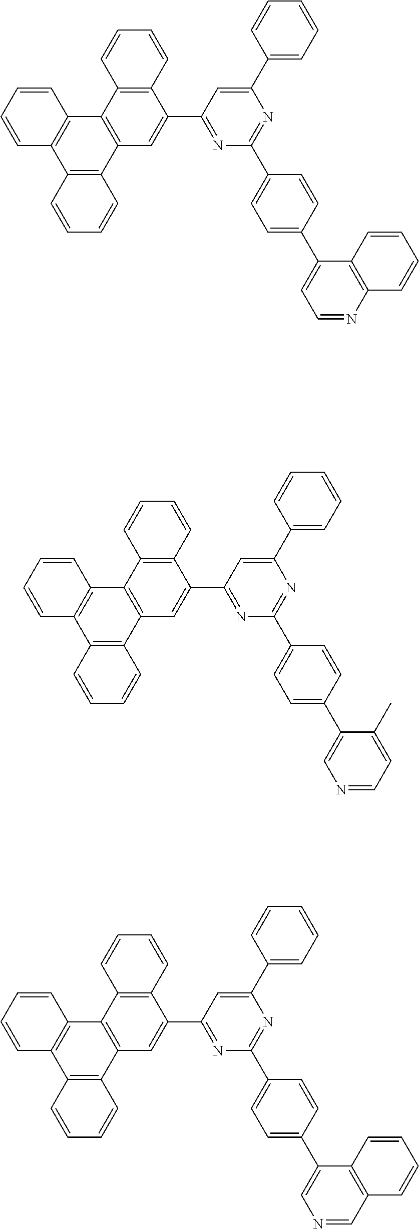 [SMILES: c1ccc(-c2cc(-c3cc4c5ccccc5c5ccccc5c4c4ccccc34)nc(-c3ccc(-c4ccnc5ccccc45)cc3)n2)cc1 ; Cc1ccncc1-c1ccc(-c2nc(-c3ccccc3)cc(-c3cc4c5ccccc5c5ccccc5c4c4ccccc34)n2)cc1 ; c1ccc(-c2cc(-c3cc4c5ccccc5c5ccccc5c4c4ccccc34)nc(-c3ccc(-c4cncc5ccccc45)cc3)n2)cc1]

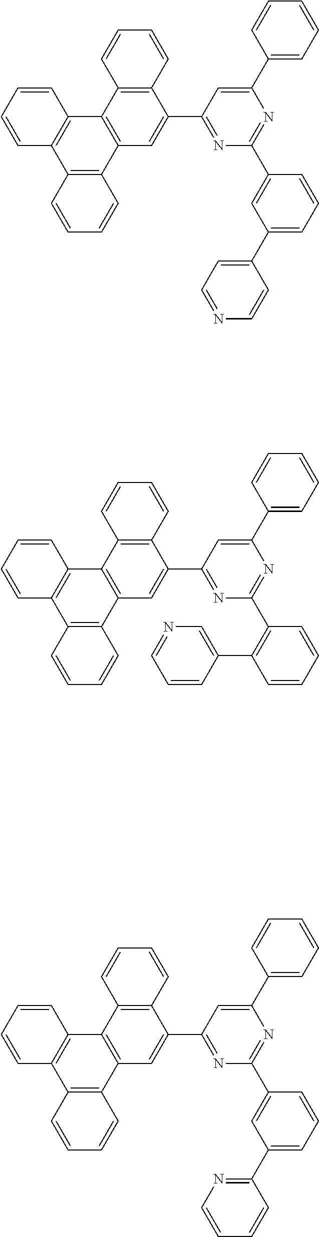 [SMILES: c1ccc(-c2cc(-c3cc4c5ccccc5c5ccccc5c4c4ccccc34)nc(-c3cccc(-c4ccncc4)c3)n2)cc1 ; c1ccc(-c2cc(-c3cc4c5ccccc5c5ccccc5c4c4ccccc34)nc(-c3ccccc3-c3cccnc3)n2)cc1 ; c1ccc(-c2cc(-c3cc4c5ccccc5c5ccccc5c4c4ccccc34)nc(-c3cccc(-c4ccccn4)c3)n2)cc1]

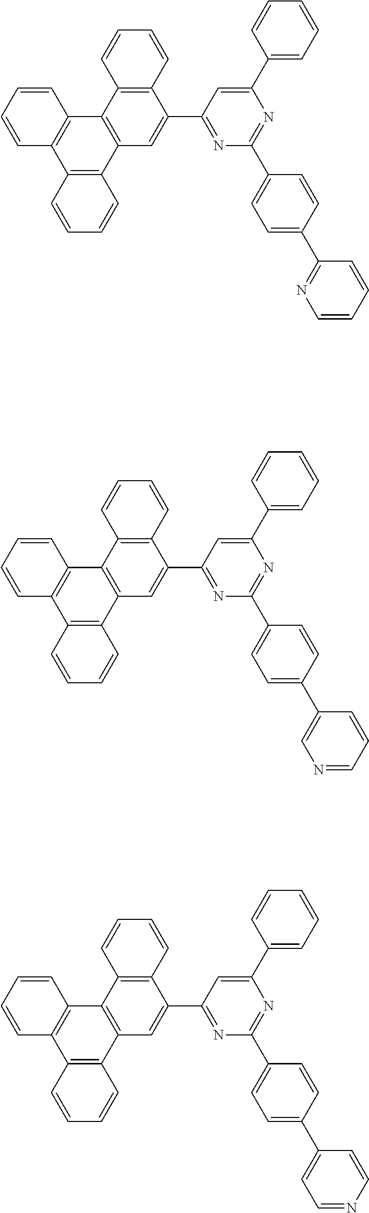 [SMILES: c1ccc(-c2cc(-c3cc4c5ccccc5c5ccccc5c4c4ccccc34)nc(-c3ccc(-c4ccccn4)cc3)n2)cc1 ; c1ccc(-c2cc(-c3cc4c5ccccc5c5ccccc5c4c4ccccc34)nc(-c3ccc(-c4cccnc4)cc3)n2)cc1 ; c1ccc(-c2cc(-c3cc4c5ccccc5c5ccccc5c4c4ccccc34)nc(-c3ccc(-c4ccncc4)cc3)n2)cc1]

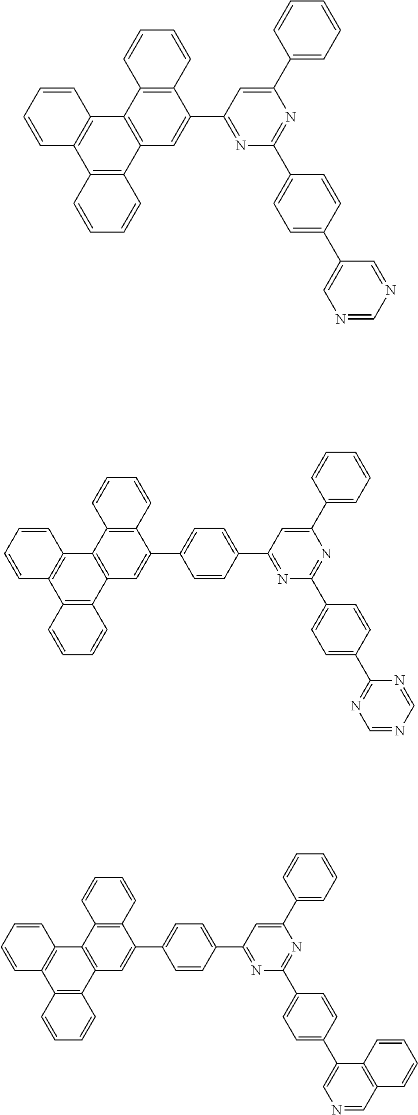 [SMILES: c1ccc(-c2cc(-c3cc4c5ccccc5c5ccccc5c4c4ccccc34)nc(-c3ccc(-c4cncnc4)cc3)n2)cc1 ; c1ccc(-c2cc(-c3ccc(-c4cc5c6ccccc6c6ccccc6c5c5ccccc45)cc3)nc(-c3ccc(-c4ncncn4)cc3)n2)cc1 ; c1ccc(-c2cc(-c3ccc(-c4cc5c6ccccc6c6ccccc6c5c5ccccc45)cc3)nc(-c3ccc(-c4cncc5ccccc45)cc3)n2)cc1]

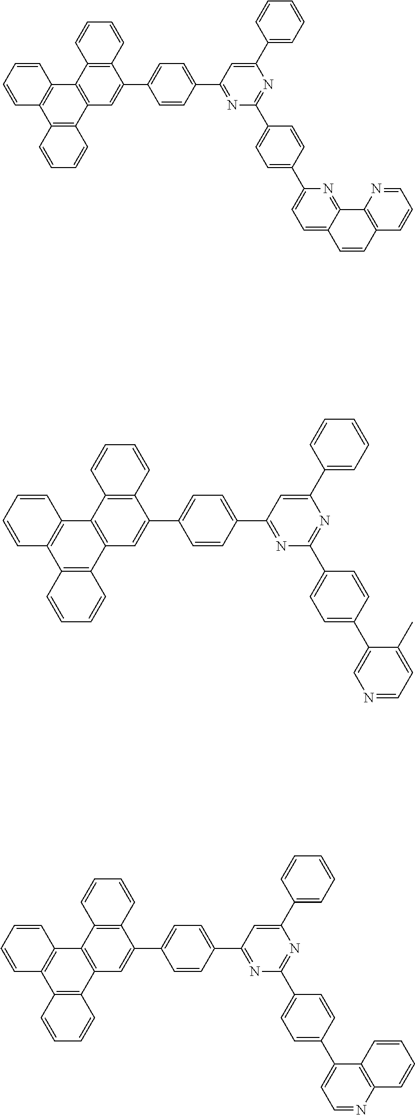 [SMILES: c1ccc(-c2cc(-c3ccc(-c4cc5c6ccccc6c6ccccc6c5c5ccccc45)cc3)nc(-c3ccc(-c4ccc5ccc6cccnc6c5n4)cc3)n2)cc1 ; Cc1ccncc1-c1ccc(-c2nc(-c3ccccc3)cc(-c3ccc(-c4cc5c6ccccc6c6ccccc6c5c5ccccc45)cc3)n2)cc1 ; c1ccc(-c2cc(-c3ccc(-c4cc5c6ccccc6c6ccccc6c5c5ccccc45)cc3)nc(-c3ccc(-c4ccnc5ccccc45)cc3)n2)cc1]

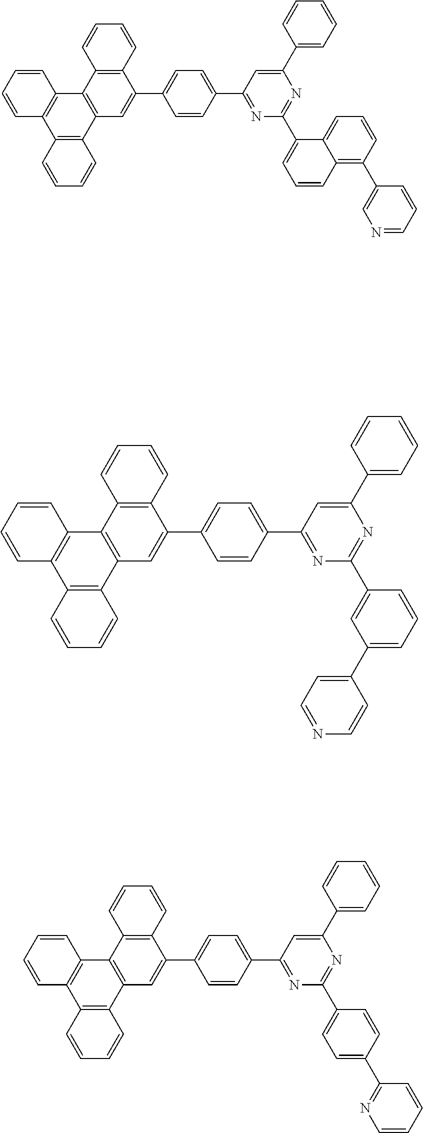 [SMILES: c1ccc(-c2cc(-c3ccc(-c4cc5c6ccccc6c6ccccc6c5c5ccccc45)cc3)nc(-c3cccc4c(-c5cccnc5)cccc34)n2)cc1 ; c1ccc(-c2cc(-c3ccc(-c4cc5c6ccccc6c6ccccc6c5c5ccccc45)cc3)nc(-c3cccc(-c4ccncc4)c3)n2)cc1 ; c1ccc(-c2cc(-c3ccc(-c4cc5c6ccccc6c6ccccc6c5c5ccccc45)cc3)nc(-c3ccc(-c4ccccn4)cc3)n2)cc1]

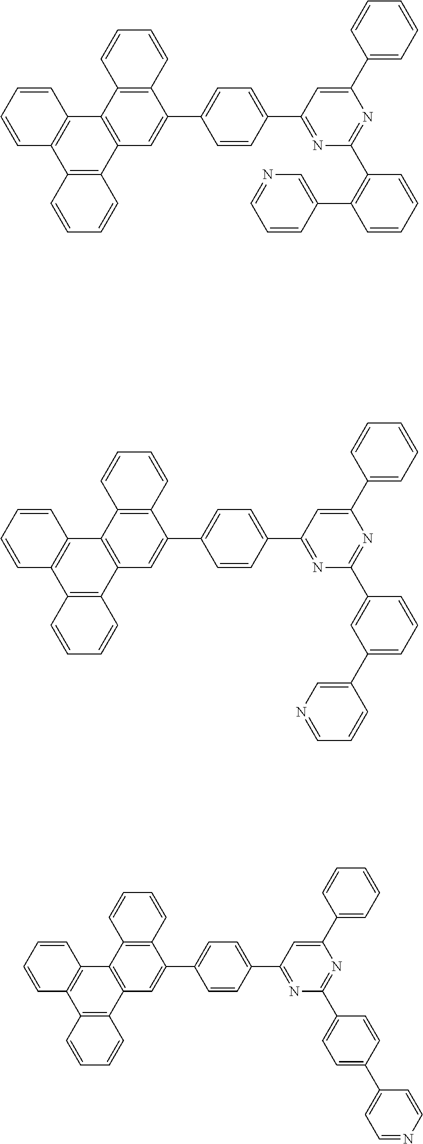 [SMILES: c1ccc(-c2cc(-c3ccc(-c4cc5c6ccccc6c6ccccc6c5c5ccccc45)cc3)nc(-c3ccccc3-c3cccnc3)n2)cc1 ; c1ccc(-c2cc(-c3ccc(-c4cc5c6ccccc6c6ccccc6c5c5ccccc45)cc3)nc(-c3cccc(-c4cccnc4)c3)n2)cc1 ; c1ccc(-c2cc(-c3ccc(-c4cc5c6ccccc6c6ccccc6c5c5ccccc45)cc3)nc(-c3ccc(-c4ccncc4)cc3)n2)cc1]

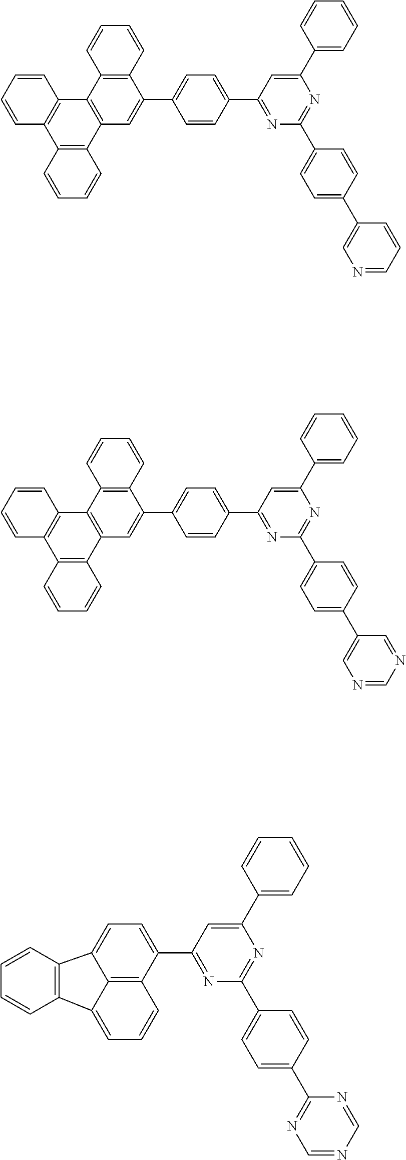 [SMILES: c1ccc(-c2cc(-c3ccc(-c4cc5c6ccccc6c6ccccc6c5c5ccccc45)cc3)nc(-c3ccc(-c4cccnc4)cc3)n2)cc1 ; c1ccc(-c2cc(-c3ccc(-c4cc5c6ccccc6c6ccccc6c5c5ccccc45)cc3)nc(-c3ccc(-c4cncnc4)cc3)n2)cc1 ; c1ccc(-c2cc(-c3ccc4c5c(cccc35)-c3ccccc3-4)nc(-c3ccc(-c4ncncn4)cc3)n2)cc1]

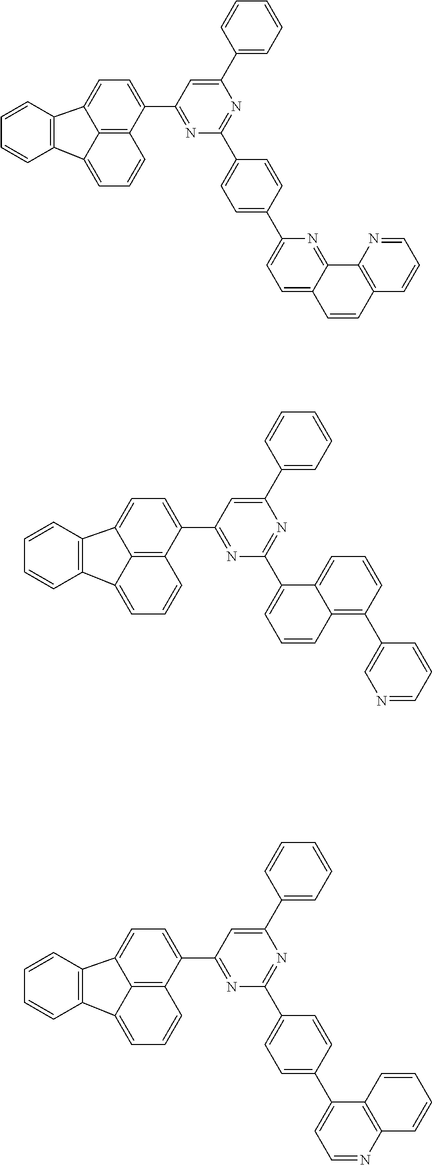 [SMILES: c1ccc(-c2cc(-c3ccc4c5c(cccc35)-c3ccccc3-4)nc(-c3ccc(-c4ccc5ccc6cccnc6c5n4)cc3)n2)cc1 ; c1ccc(-c2cc(-c3ccc4c5c(cccc35)-c3ccccc3-4)nc(-c3cccc4c(-c5cccnc5)cccc34)n2)cc1 ; c1ccc(-c2cc(-c3ccc4c5c(cccc35)-c3ccccc3-4)nc(-c3ccc(-c4ccnc5ccccc45)cc3)n2)cc1]

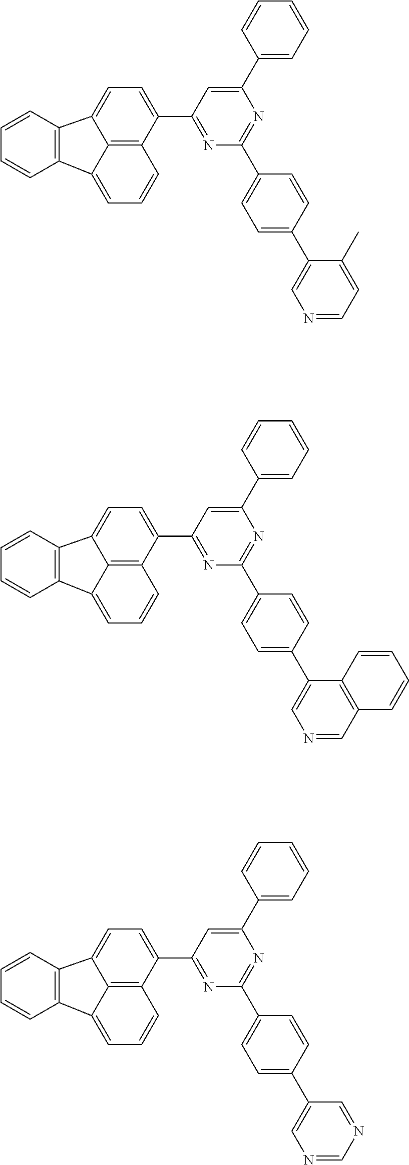 [SMILES: Cc1ccncc1-c1ccc(-c2nc(-c3ccccc3)cc(-c3ccc4c5c(cccc35)-c3ccccc3-4)n2)cc1 ; c1ccc(-c2cc(-c3ccc4c5c(cccc35)-c3ccccc3-4)nc(-c3ccc(-c4cncc5ccccc45)cc3)n2)cc1 ; c1ccc(-c2cc(-c3ccc4c5c(cccc35)-c3ccccc3-4)nc(-c3ccc(-c4cncnc4)cc3)n2)cc1]

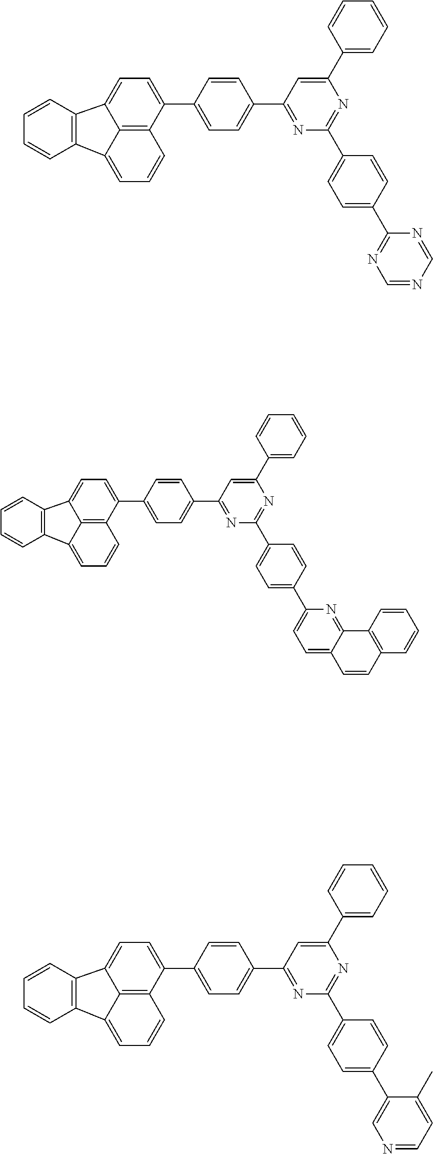 [SMILES: c1ccc(-c2cc(-c3ccc(-c4ccc5c6c(cccc46)-c4ccccc4-5)cc3)nc(-c3ccc(-c4ncncn4)cc3)n2)cc1 ; c1ccc(-c2cc(-c3ccc(-c4ccc5c6c(cccc46)-c4ccccc4-5)cc3)nc(-c3ccc(-c4ccc5ccc6ccccc6c5n4)cc3)n2)cc1 ; Cc1ccncc1-c1ccc(-c2nc(-c3ccccc3)cc(-c3ccc(-c4ccc5c6c(cccc46)-c4ccccc4-5)cc3)n2)cc1]

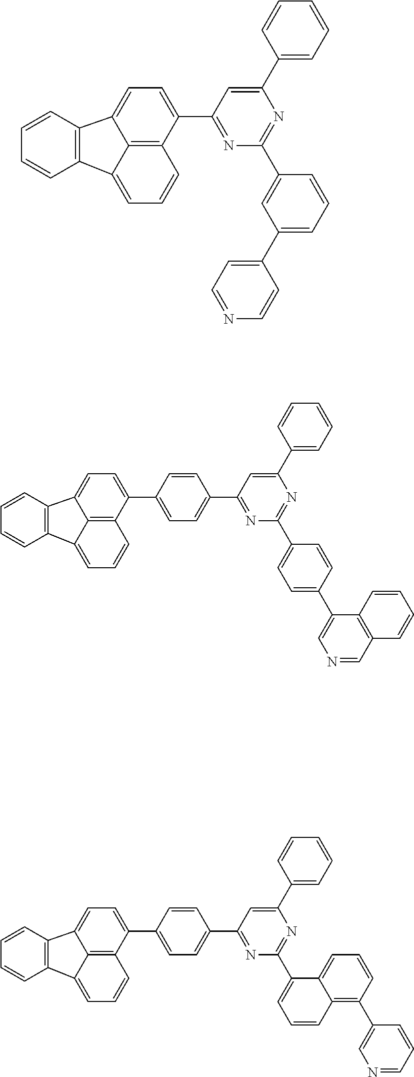 [SMILES: c1ccc(-c2cc(-c3ccc4c5c(cccc35)-c3ccccc3-4)nc(-c3cccc(-c4ccncc4)c3)n2)cc1 ; c1ccc(-c2cc(-c3ccc(-c4ccc5c6c(cccc46)-c4ccccc4-5)cc3)nc(-c3ccc(-c4cncc5ccccc45)cc3)n2)cc1 ; c1ccc(-c2cc(-c3ccc(-c4ccc5c6c(cccc46)-c4ccccc4-5)cc3)nc(-c3cccc4c(-c5cccnc5)cccc34)n2)cc1]

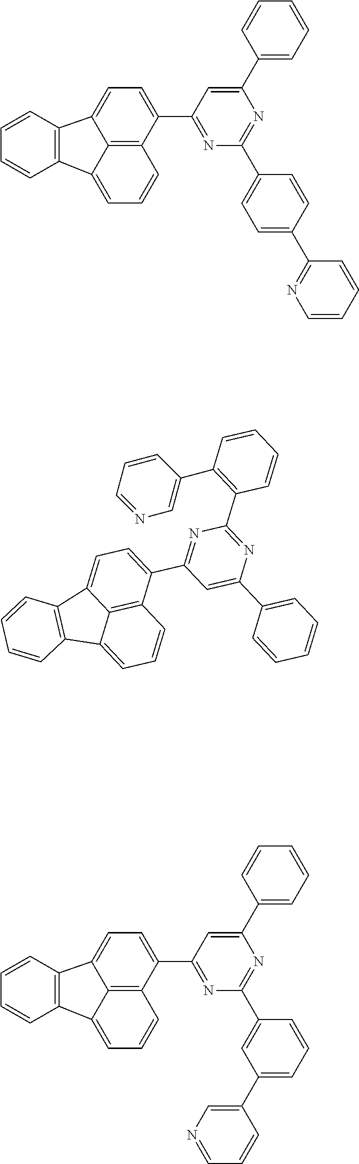 [SMILES: c1ccc(-c2cc(-c3ccc4c5c(cccc35)-c3ccccc3-4)nc(-c3ccc(-c4ccccn4)cc3)n2)cc1 ; c1ccc(-c2cc(-c3ccc4c5c(cccc35)-c3ccccc3-4)nc(-c3ccccc3-c3cccnc3)n2)cc1 ; c1ccc(-c2cc(-c3ccc4c5c(cccc35)-c3ccccc3-4)nc(-c3cccc(-c4cccnc4)c3)n2)cc1]

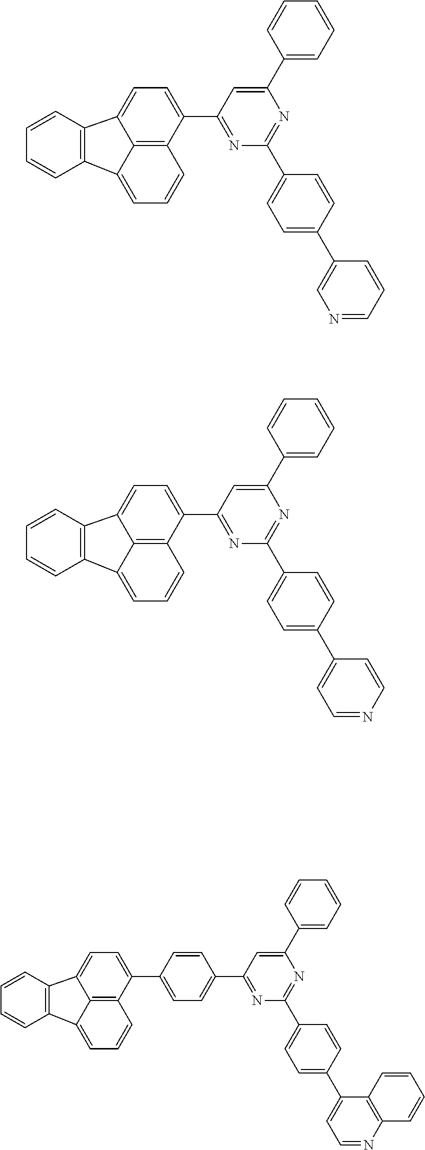 [SMILES: c1ccc(-c2cc(-c3ccc4c5c(cccc35)-c3ccccc3-4)nc(-c3ccc(-c4cccnc4)cc3)n2)cc1 ; c1ccc(-c2cc(-c3ccc4c5c(cccc35)-c3ccccc3-4)nc(-c3ccc(-c4ccncc4)cc3)n2)cc1 ; c1ccc(-c2cc(-c3ccc(-c4ccc5c6c(cccc46)-c4ccccc4-5)cc3)nc(-c3ccc(-c4ccnc5ccccc45)cc3)n2)cc1]

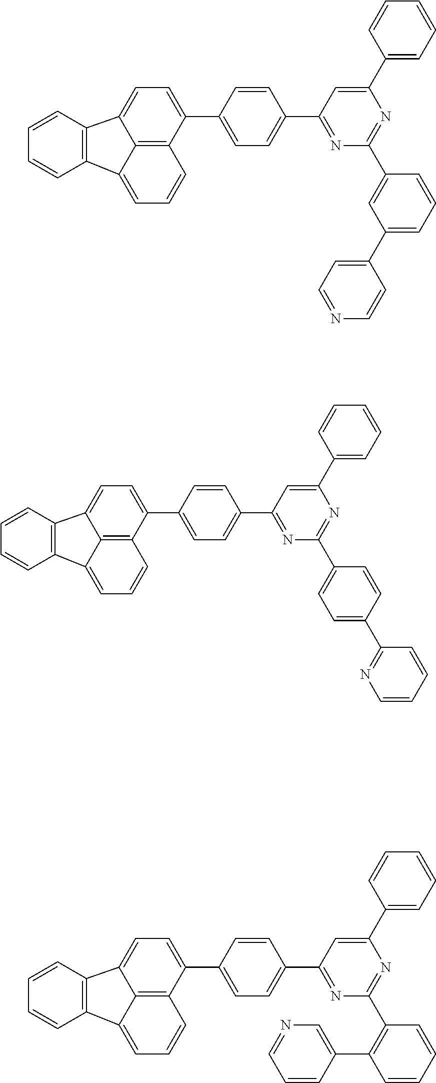 [SMILES: c1ccc(-c2cc(-c3ccc(-c4ccc5c6c(cccc46)-c4ccccc4-5)cc3)nc(-c3cccc(-c4ccncc4)c3)n2)cc1 ; c1ccc(-c2cc(-c3ccc(-c4ccc5c6c(cccc46)-c4ccccc4-5)cc3)nc(-c3ccc(-c4ccccn4)cc3)n2)cc1 ; c1ccc(-c2cc(-c3ccc(-c4ccc5c6c(cccc46)-c4ccccc4-5)cc3)nc(-c3ccccc3-c3cccnc3)n2)cc1]

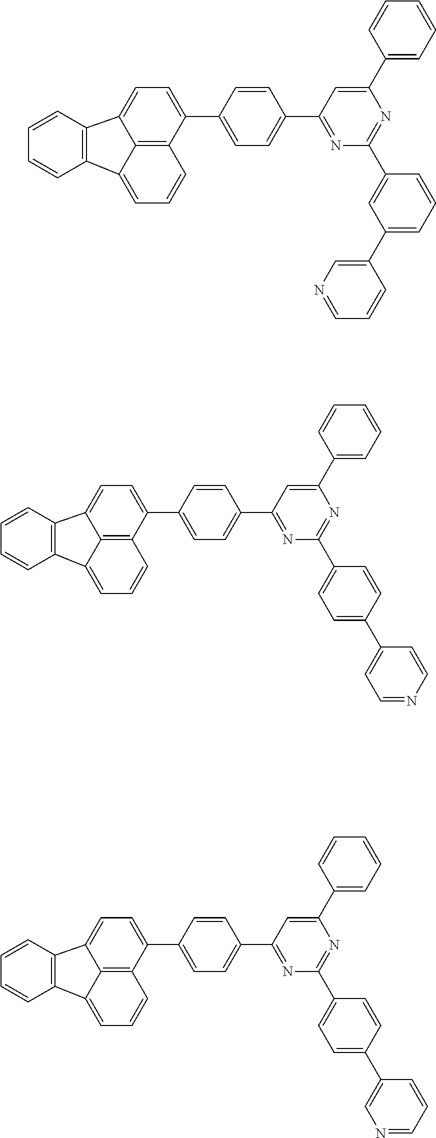 [SMILES: c1ccc(-c2cc(-c3ccc(-c4ccc5c6c(cccc46)-c4ccccc4-5)cc3)nc(-c3cccc(-c4cccnc4)c3)n2)cc1 ; c1ccc(-c2cc(-c3ccc(-c4ccc5c6c(cccc46)-c4ccccc4-5)cc3)nc(-c3ccc(-c4ccncc4)cc3)n2)cc1 ; c1ccc(-c2cc(-c3ccc(-c4ccc5c6c(cccc46)-c4ccccc4-5)cc3)nc(-c3ccc(-c4cccnc4)cc3)n2)cc1]

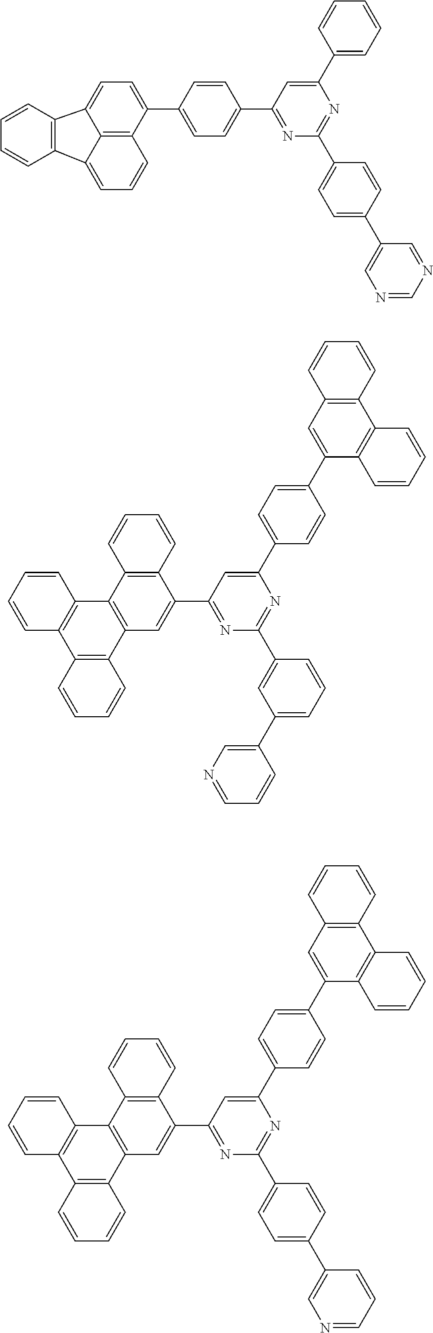 [SMILES: c1ccc(-c2cc(-c3ccc(-c4ccc5c6c(cccc46)-c4ccccc4-5)cc3)nc(-c3ccc(-c4cncnc4)cc3)n2)cc1 ; c1cncc(-c2cccc(-c3nc(-c4ccc(-c5cc6ccccc6c6ccccc56)cc4)cc(-c4cc5c6ccccc6c6ccccc6c5c5ccccc45)n3)c2)c1 ; c1cncc(-c2ccc(-c3nc(-c4ccc(-c5cc6ccccc6c6ccccc56)cc4)cc(-c4cc5c6ccccc6c6ccccc6c5c5ccccc45)n3)cc2)c1]

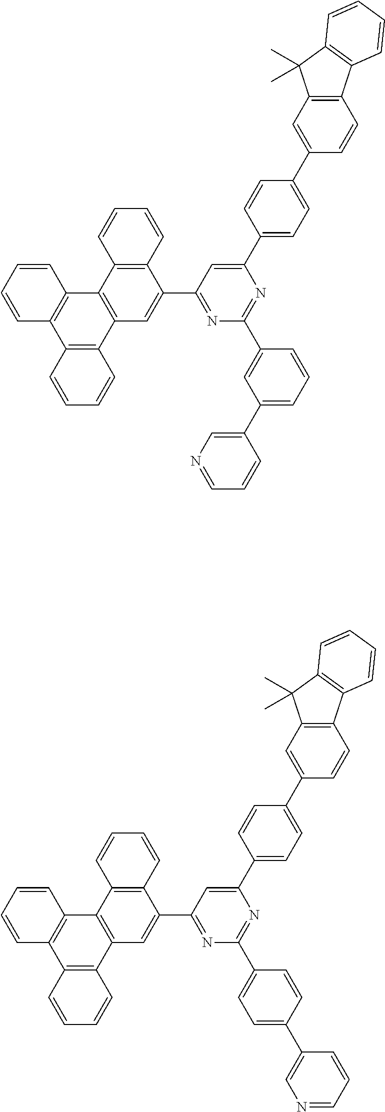 [SMILES: CC1(C)c2ccccc2-c2ccc(-c3ccc(-c4cc(-c5cc6c7ccccc7c7ccccc7c6c6ccccc56)nc(-c5cccc(-c6cccnc6)c5)n4)cc3)cc21 ; CC1(C)c2ccccc2-c2ccc(-c3ccc(-c4cc(-c5cc6c7ccccc7c7ccccc7c6c6ccccc56)nc(-c5ccc(-c6cccnc6)cc5)n4)cc3)cc21]

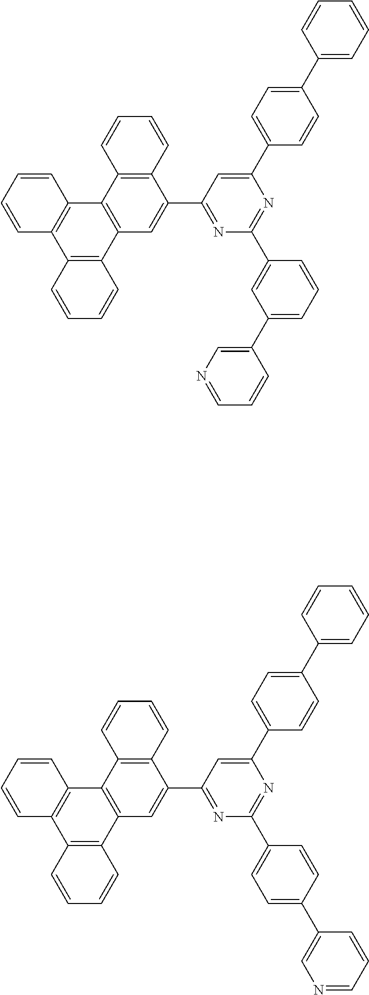 [SMILES: c1ccc(-c2ccc(-c3cc(-c4cc5c6ccccc6c6ccccc6c5c5ccccc45)nc(-c4cccc(-c5cccnc5)c4)n3)cc2)cc1 ; c1ccc(-c2ccc(-c3cc(-c4cc5c6ccccc6c6ccccc6c5c5ccccc45)nc(-c4ccc(-c5cccnc5)cc4)n3)cc2)cc1]

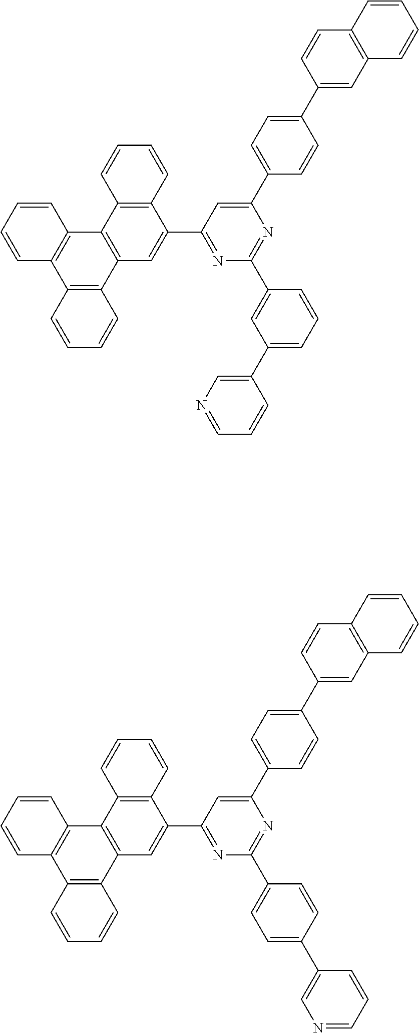 [SMILES: c1cncc(-c2cccc(-c3nc(-c4ccc(-c5ccc6ccccc6c5)cc4)cc(-c4cc5c6ccccc6c6ccccc6c5c5ccccc45)n3)c2)c1 ; c1cncc(-c2ccc(-c3nc(-c4ccc(-c5ccc6ccccc6c5)cc4)cc(-c4cc5c6ccccc6c6ccccc6c5c5ccccc45)n3)cc2)c1]

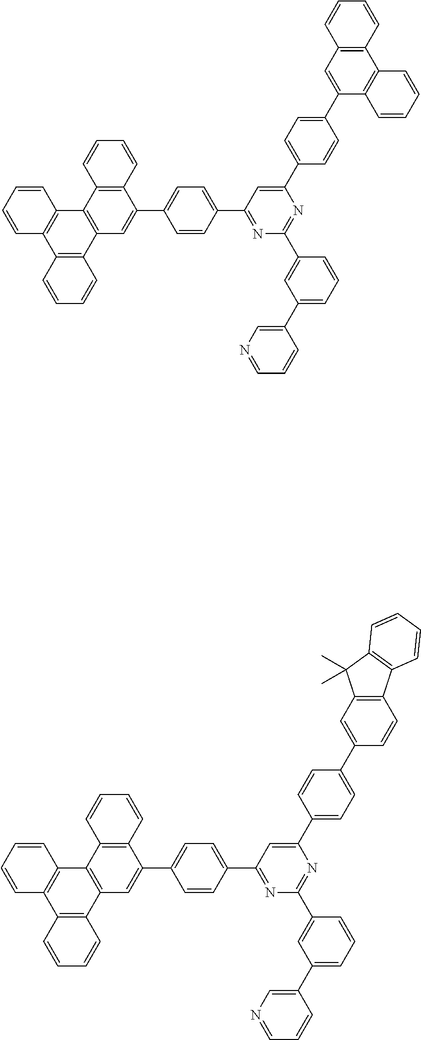 [SMILES: c1cncc(-c2cccc(-c3nc(-c4ccc(-c5cc6ccccc6c6ccccc56)cc4)cc(-c4ccc(-c5cc6c7ccccc7c7ccccc7c6c6ccccc56)cc4)n3)c2)c1 ; CC1(C)c2ccccc2-c2ccc(-c3ccc(-c4cc(-c5ccc(-c6cc7c8ccccc8c8ccccc8c7c7ccccc67)cc5)nc(-c5cccc(-c6cccnc6)c5)n4)cc3)cc21]

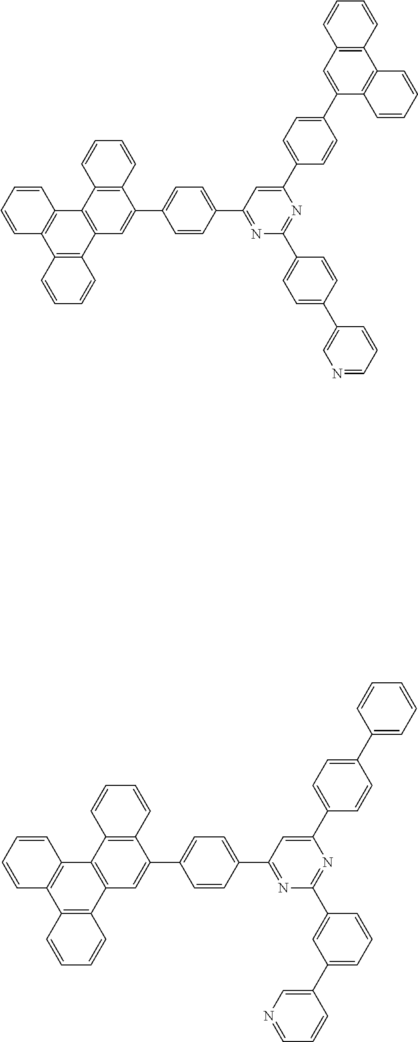 [SMILES: c1cncc(-c2ccc(-c3nc(-c4ccc(-c5cc6ccccc6c6ccccc56)cc4)cc(-c4ccc(-c5cc6c7ccccc7c7ccccc7c6c6ccccc56)cc4)n3)cc2)c1 ; c1ccc(-c2ccc(-c3cc(-c4ccc(-c5cc6c7ccccc7c7ccccc7c6c6ccccc56)cc4)nc(-c4cccc(-c5cccnc5)c4)n3)cc2)cc1]

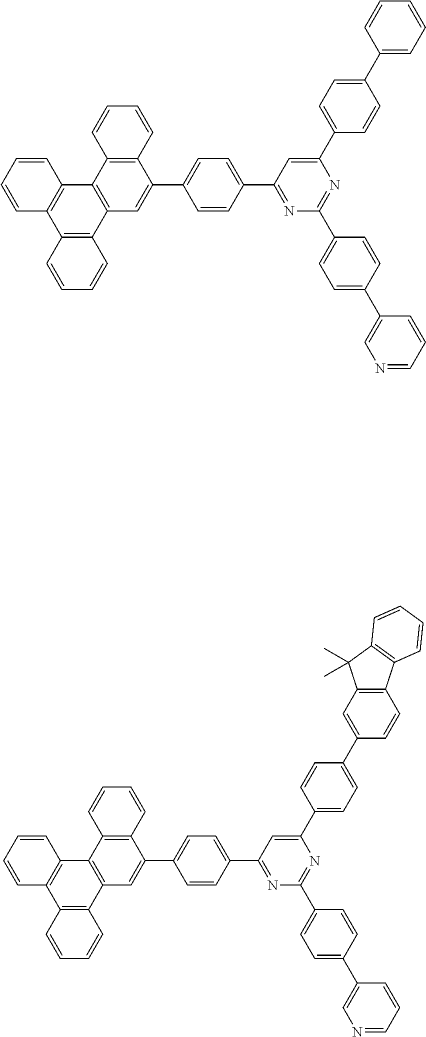 [SMILES: c1ccc(-c2ccc(-c3cc(-c4ccc(-c5cc6c7ccccc7c7ccccc7c6c6ccccc56)cc4)nc(-c4ccc(-c5cccnc5)cc4)n3)cc2)cc1 ; CC1(C)c2ccccc2-c2ccc(-c3ccc(-c4cc(-c5ccc(-c6cc7c8ccccc8c8ccccc8c7c7ccccc67)cc5)nc(-c5ccc(-c6cccnc6)cc5)n4)cc3)cc21]

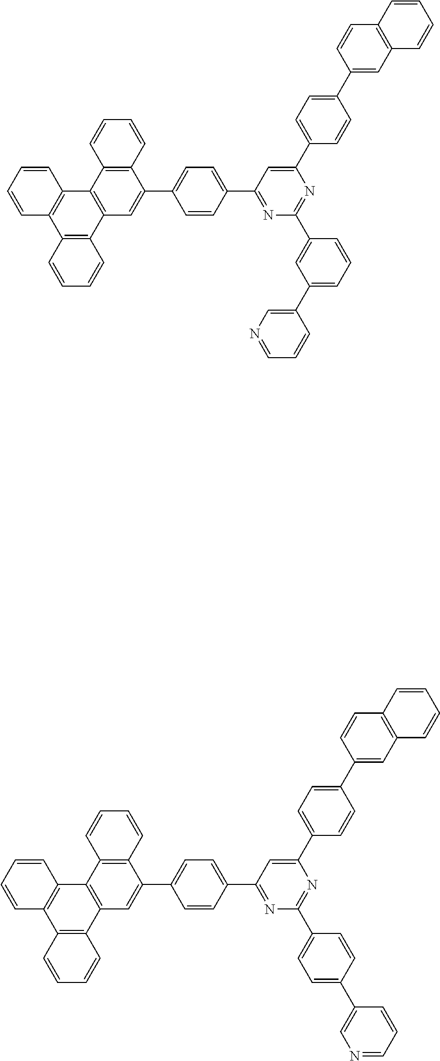 [SMILES: c1cncc(-c2cccc(-c3nc(-c4ccc(-c5ccc6ccccc6c5)cc4)cc(-c4ccc(-c5cc6c7ccccc7c7ccccc7c6c6ccccc56)cc4)n3)c2)c1 ; c1cncc(-c2ccc(-c3nc(-c4ccc(-c5ccc6ccccc6c5)cc4)cc(-c4ccc(-c5cc6c7ccccc7c7ccccc7c6c6ccccc56)cc4)n3)cc2)c1]

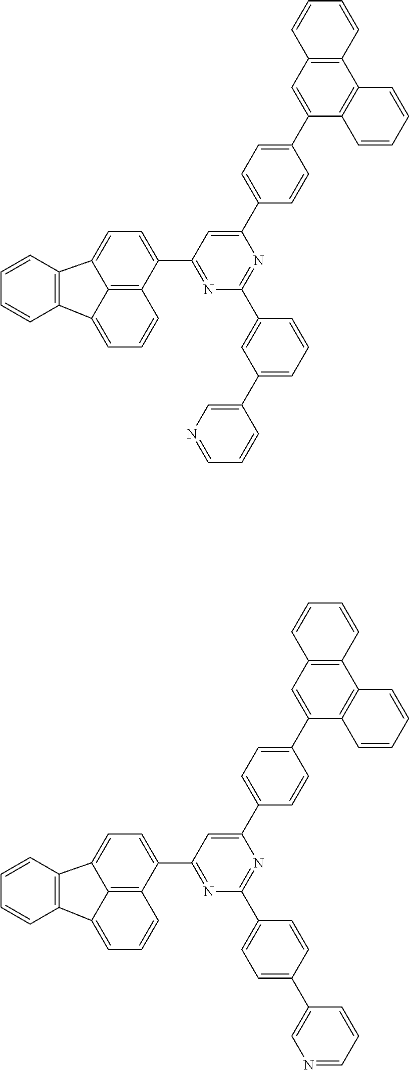 [SMILES: c1cncc(-c2cccc(-c3nc(-c4ccc(-c5cc6ccccc6c6ccccc56)cc4)cc(-c4ccc5c6c(cccc46)-c4ccccc4-5)n3)c2)c1 ; c1cncc(-c2ccc(-c3nc(-c4ccc(-c5cc6ccccc6c6ccccc56)cc4)cc(-c4ccc5c6c(cccc46)-c4ccccc4-5)n3)cc2)c1]

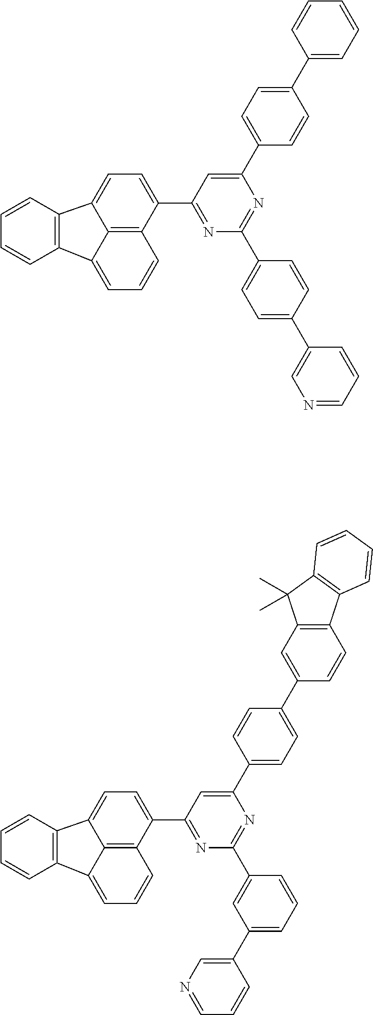 [SMILES: c1ccc(-c2ccc(-c3cc(-c4ccc5c6c(cccc46)-c4ccccc4-5)nc(-c4ccc(-c5cccnc5)cc4)n3)cc2)cc1 ; CC1(C)c2ccccc2-c2ccc(-c3ccc(-c4cc(-c5ccc6c7c(cccc57)-c5ccccc5-6)nc(-c5cccc(-c6cccnc6)c5)n4)cc3)cc21]

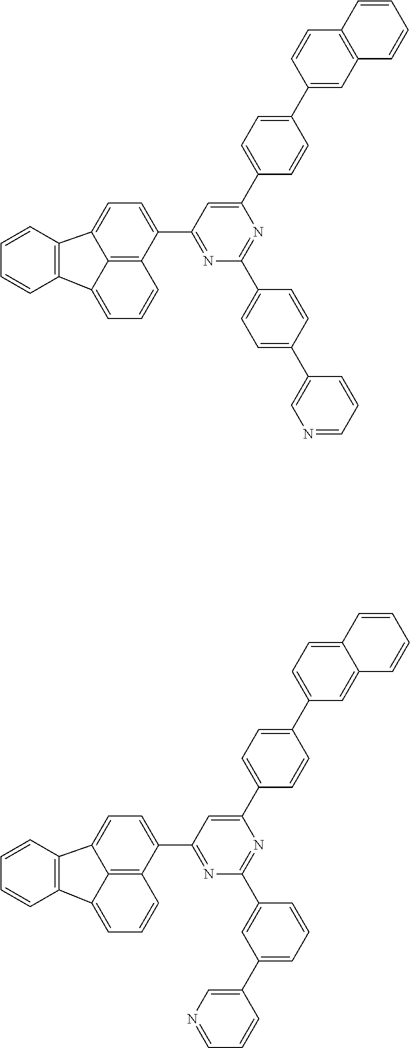 [SMILES: c1cncc(-c2ccc(-c3nc(-c4ccc(-c5ccc6ccccc6c5)cc4)cc(-c4ccc5c6c(cccc46)-c4ccccc4-5)n3)cc2)c1 ; c1cncc(-c2cccc(-c3nc(-c4ccc(-c5ccc6ccccc6c5)cc4)cc(-c4ccc5c6c(cccc46)-c4ccccc4-5)n3)c2)c1]

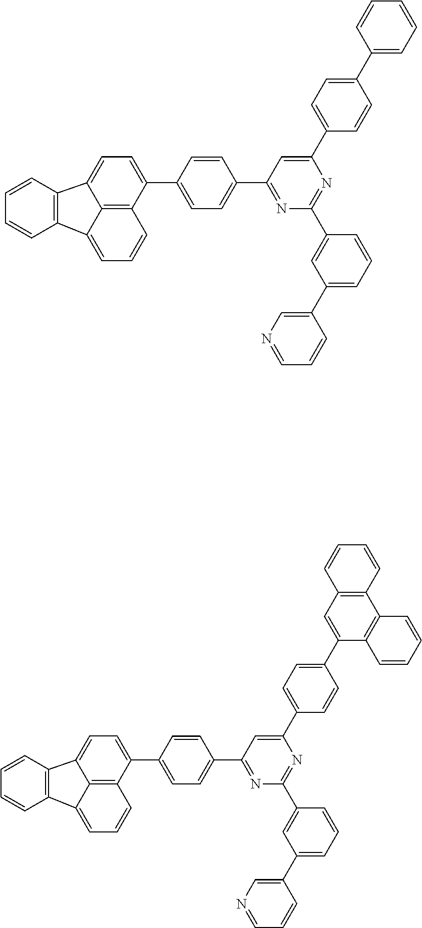 [SMILES: c1ccc(-c2ccc(-c3cc(-c4ccc(-c5ccc6c7c(cccc57)-c5ccccc5-6)cc4)nc(-c4cccc(-c5cccnc5)c4)n3)cc2)cc1 ; c1cncc(-c2cccc(-c3nc(-c4ccc(-c5ccc6c7c(cccc57)-c5ccccc5-6)cc4)cc(-c4ccc(-c5cc6ccccc6c6ccccc56)cc4)n3)c2)c1]

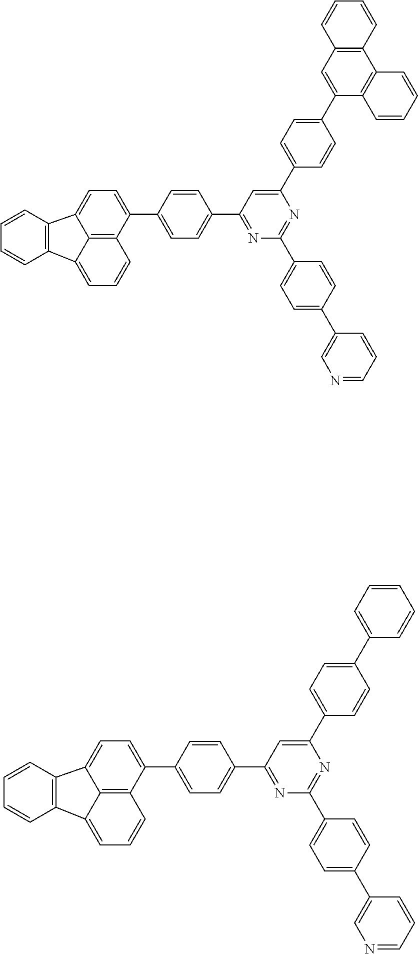 [SMILES: c1cncc(-c2ccc(-c3nc(-c4ccc(-c5ccc6c7c(cccc57)-c5ccccc5-6)cc4)cc(-c4ccc(-c5cc6ccccc6c6ccccc56)cc4)n3)cc2)c1 ; c1ccc(-c2ccc(-c3cc(-c4ccc(-c5ccc6c7c(cccc57)-c5ccccc5-6)cc4)nc(-c4ccc(-c5cccnc5)cc4)n3)cc2)cc1]

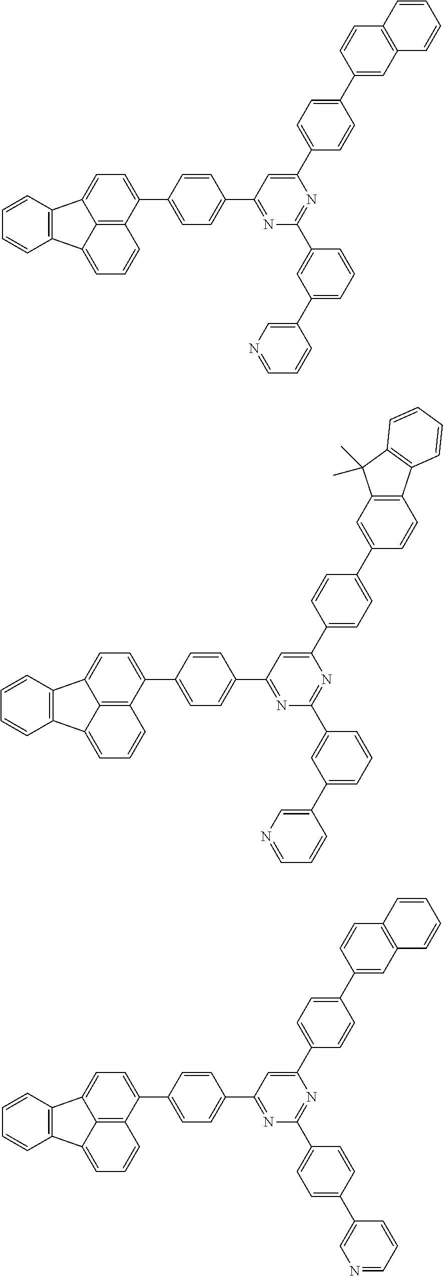 [SMILES: c1cncc(-c2cccc(-c3nc(-c4ccc(-c5ccc6ccccc6c5)cc4)cc(-c4ccc(-c5ccc6c7c(cccc57)-c5ccccc5-6)cc4)n3)c2)c1 ; CC1(C)c2ccccc2-c2ccc(-c3ccc(-c4cc(-c5ccc(-c6ccc7c8c(cccc68)-c6ccccc6-7)cc5)nc(-c5cccc(-c6cccnc6)c5)n4)cc3)cc21 ; c1cncc(-c2ccc(-c3nc(-c4ccc(-c5ccc6ccccc6c5)cc4)cc(-c4ccc(-c5ccc6c7c(cccc57)-c5ccccc5-6)cc4)n3)cc2)c1]

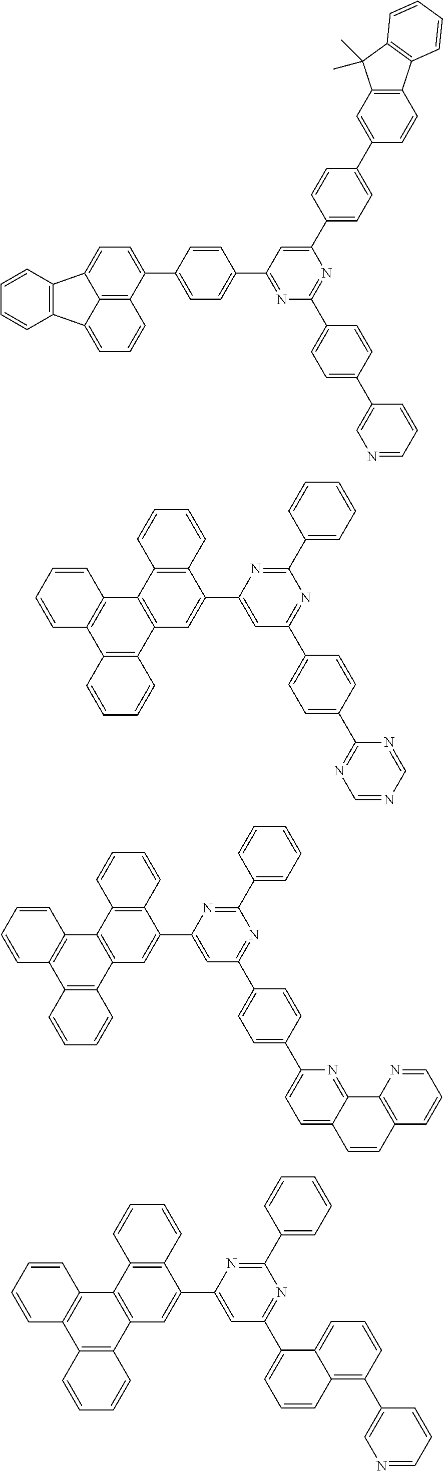

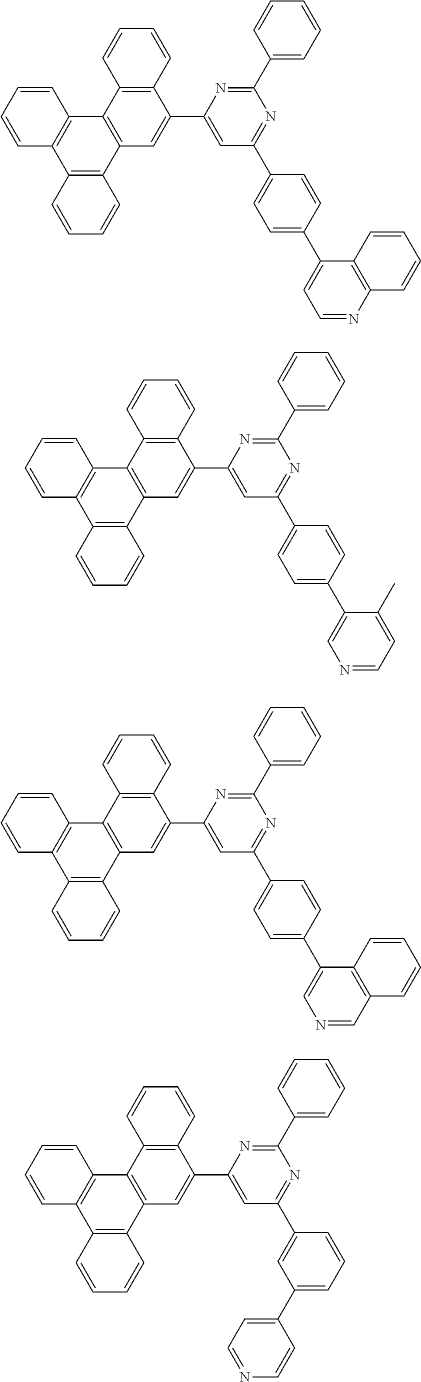

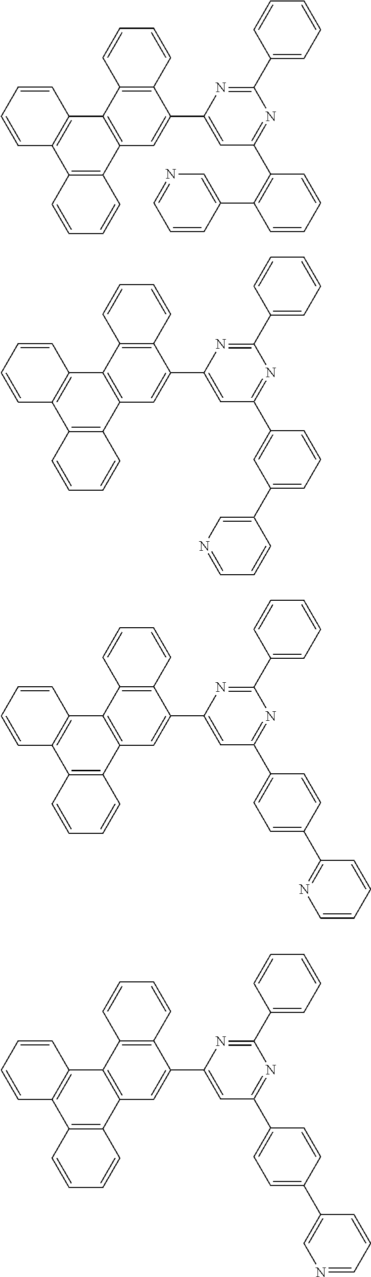

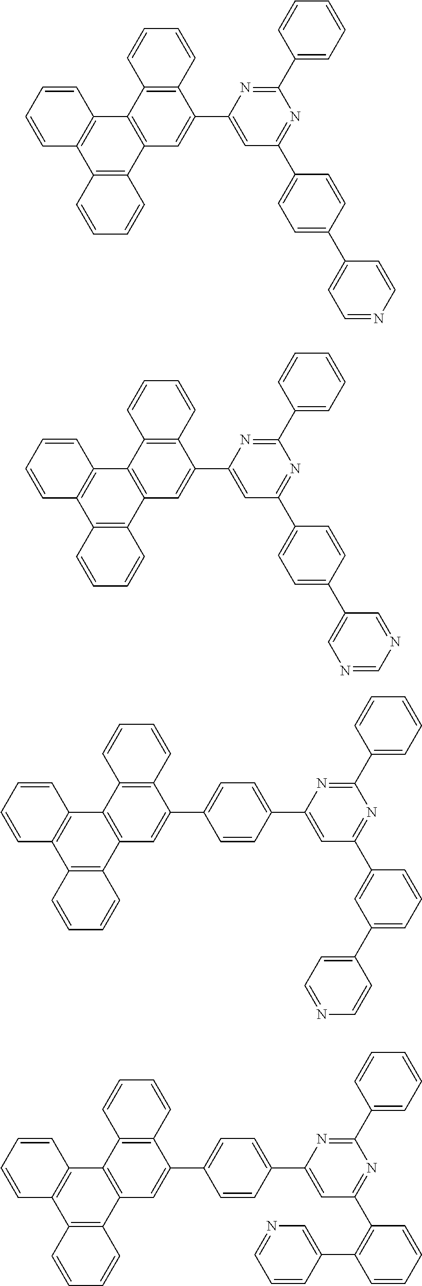

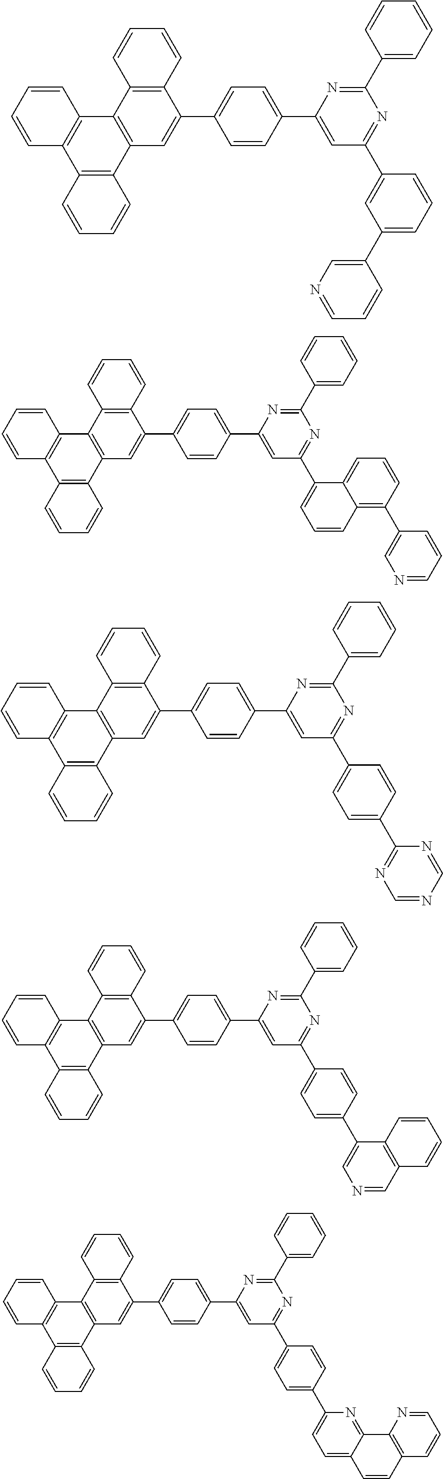

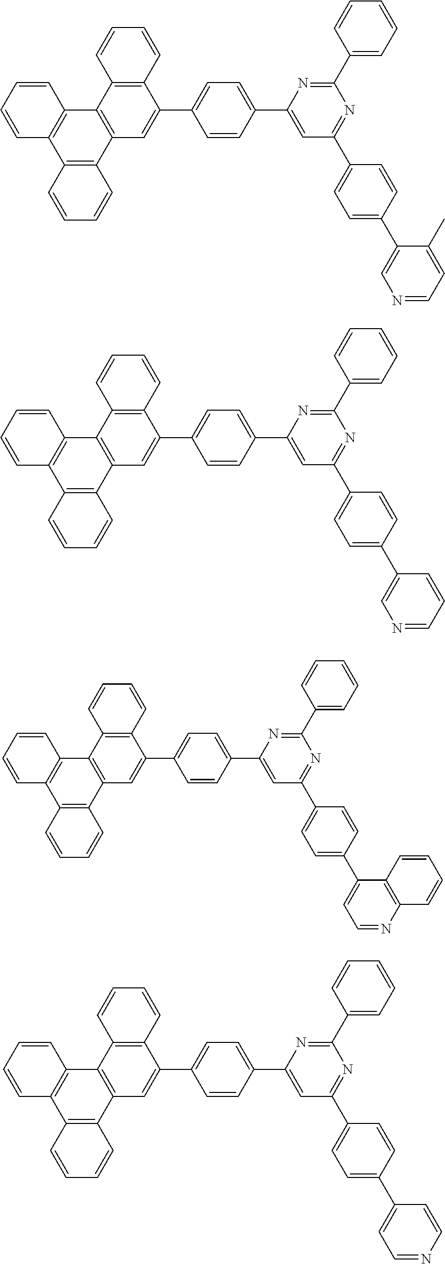

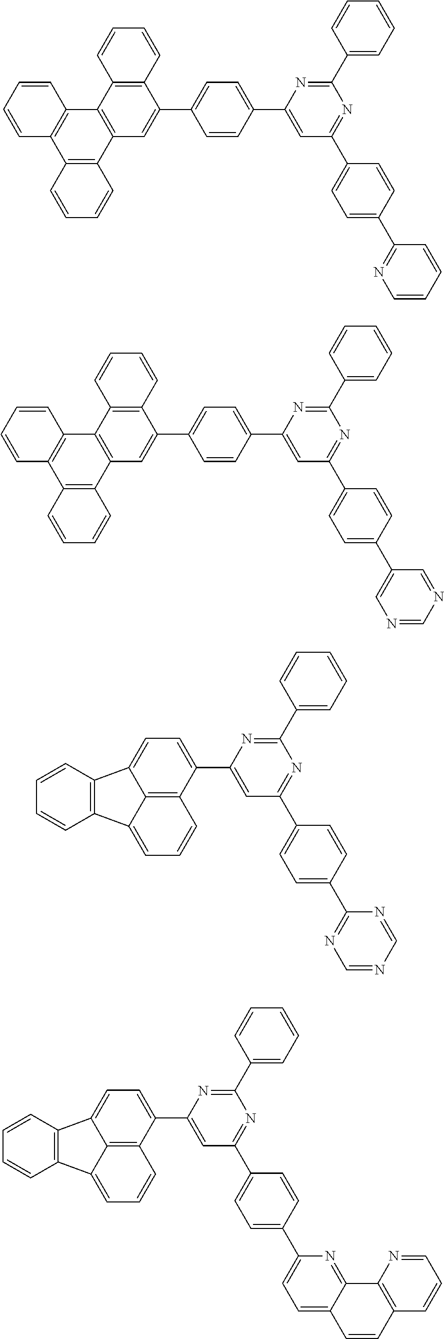

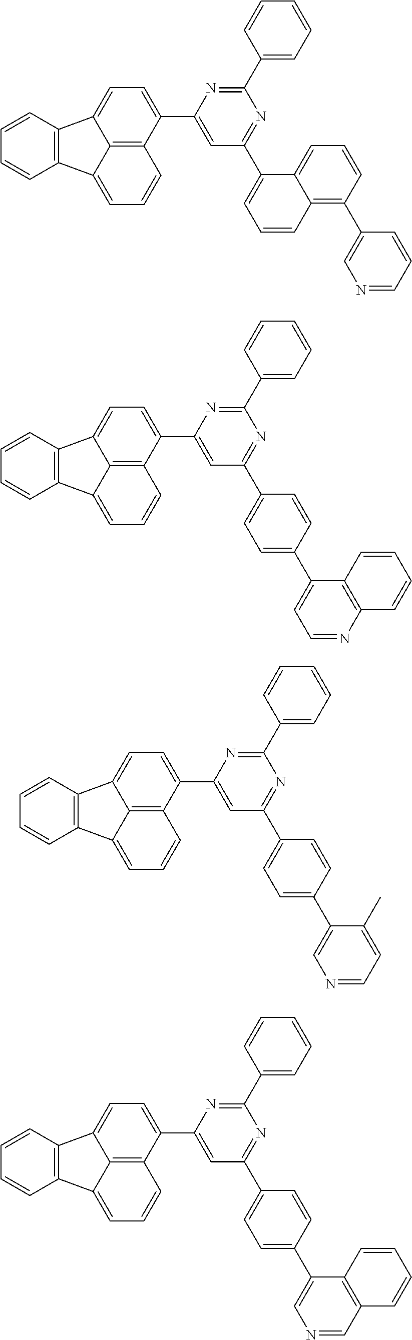

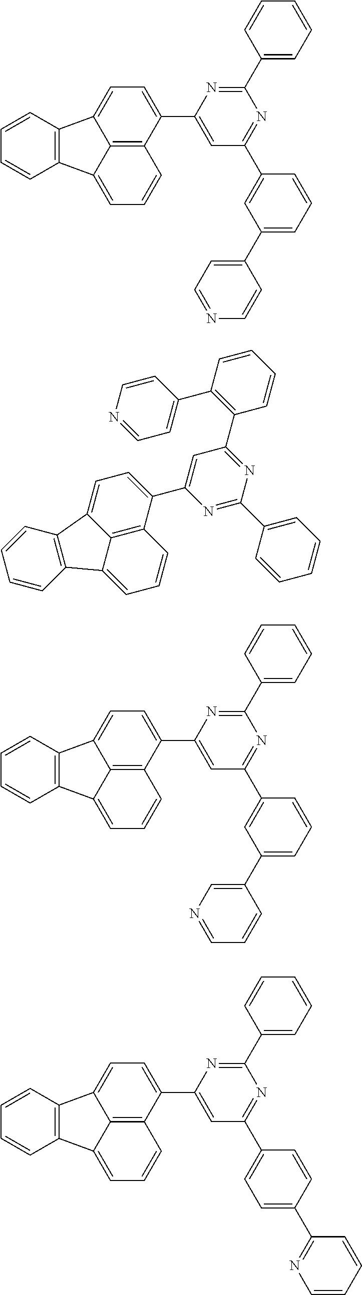

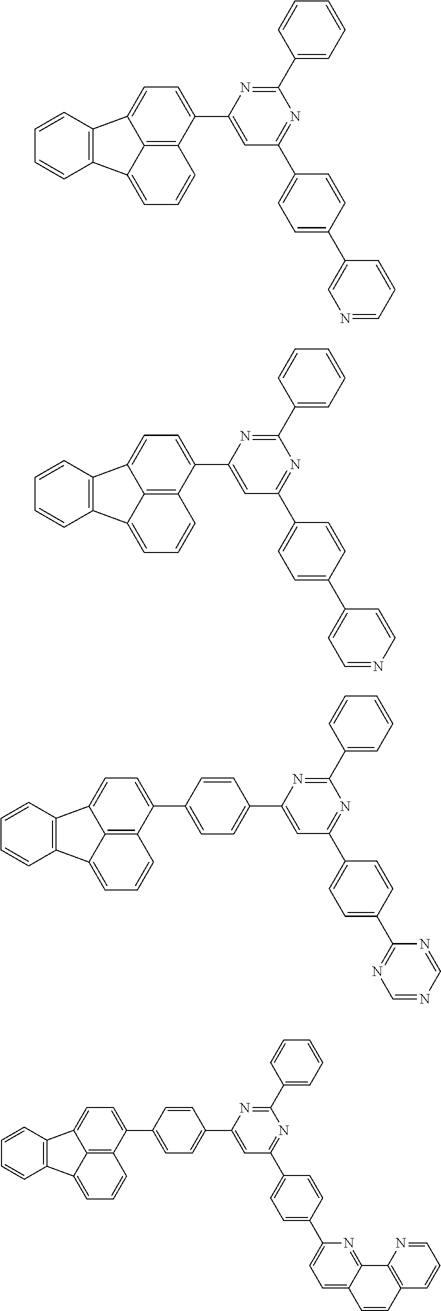

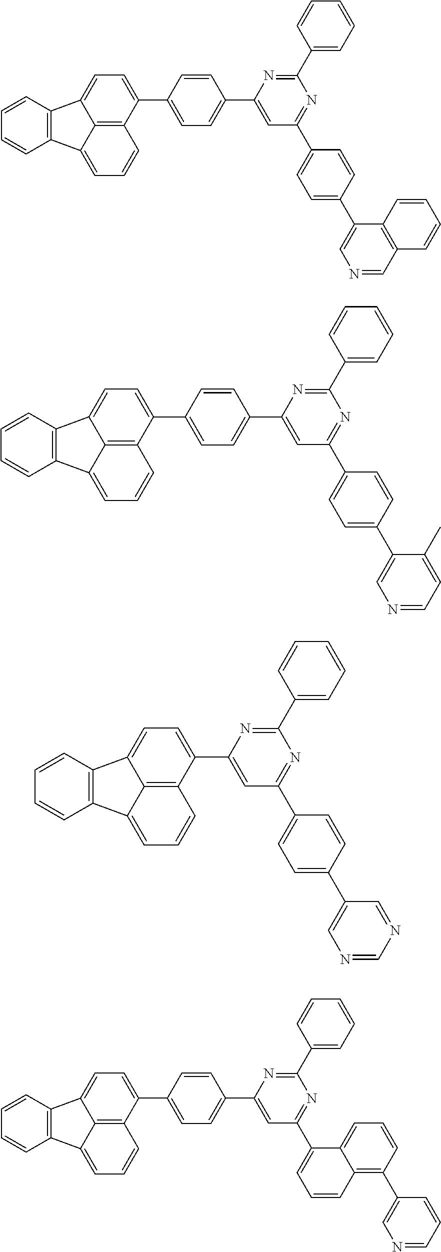

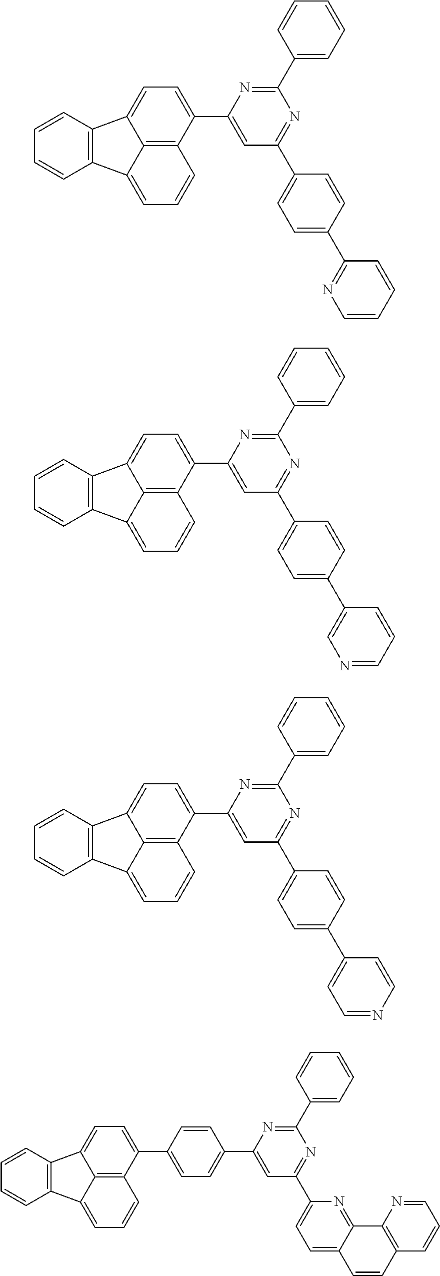

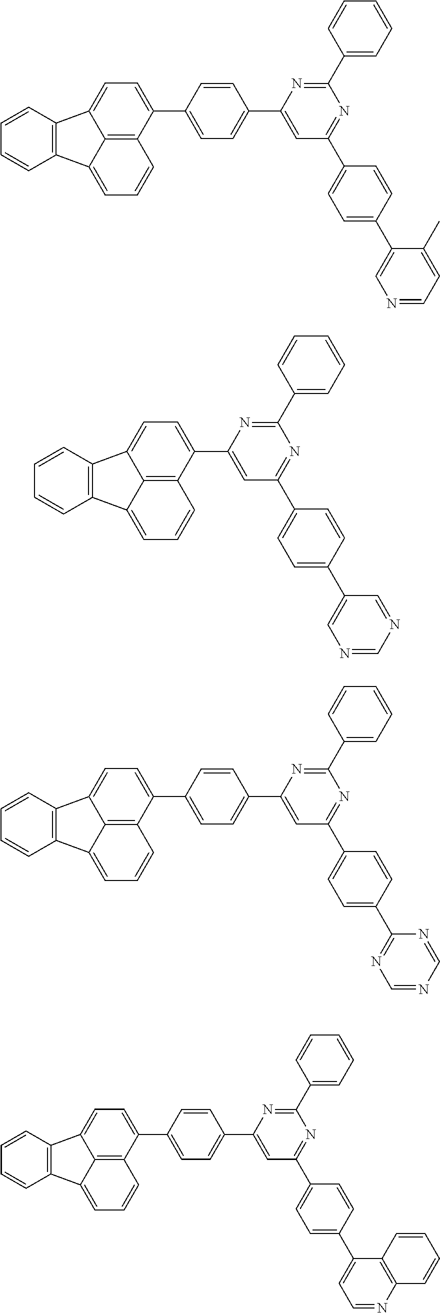

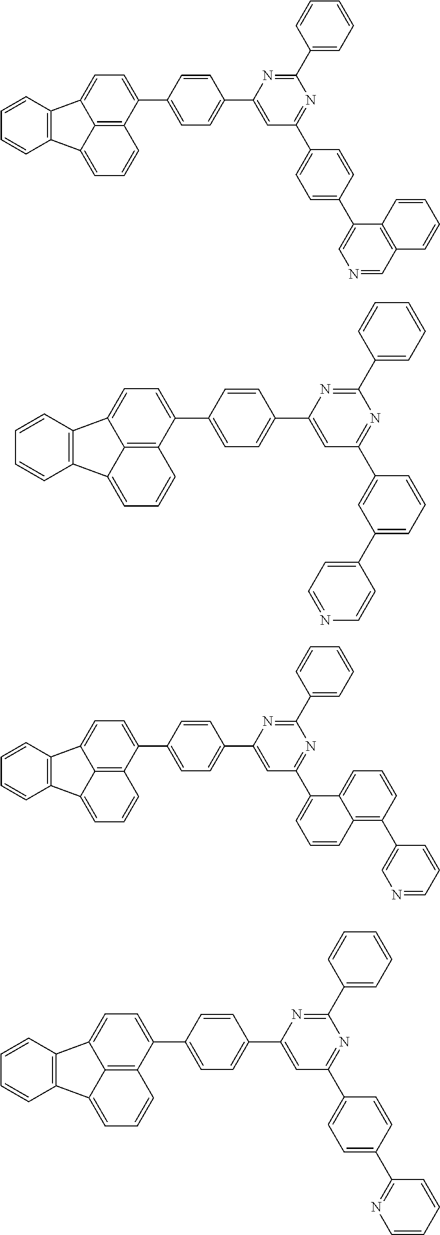

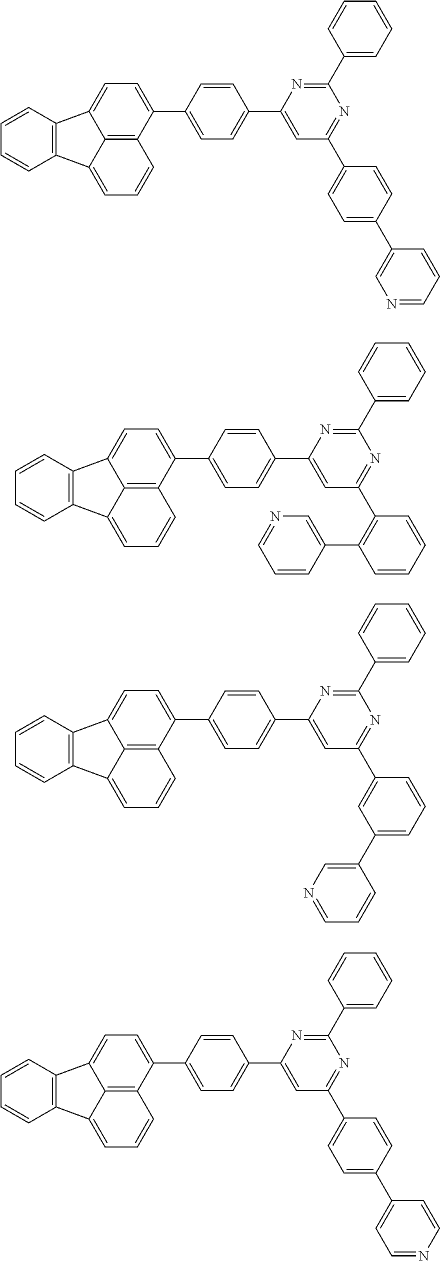

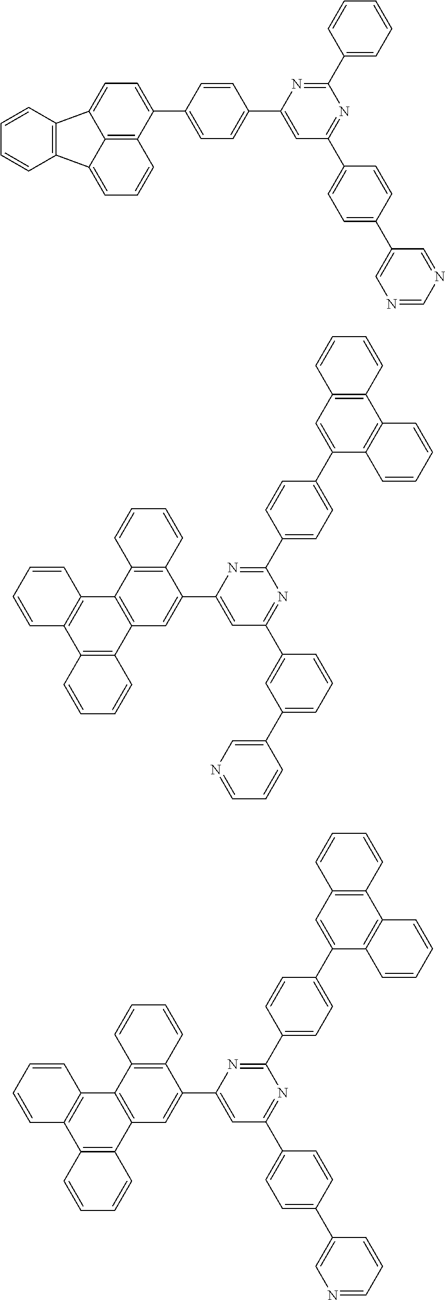

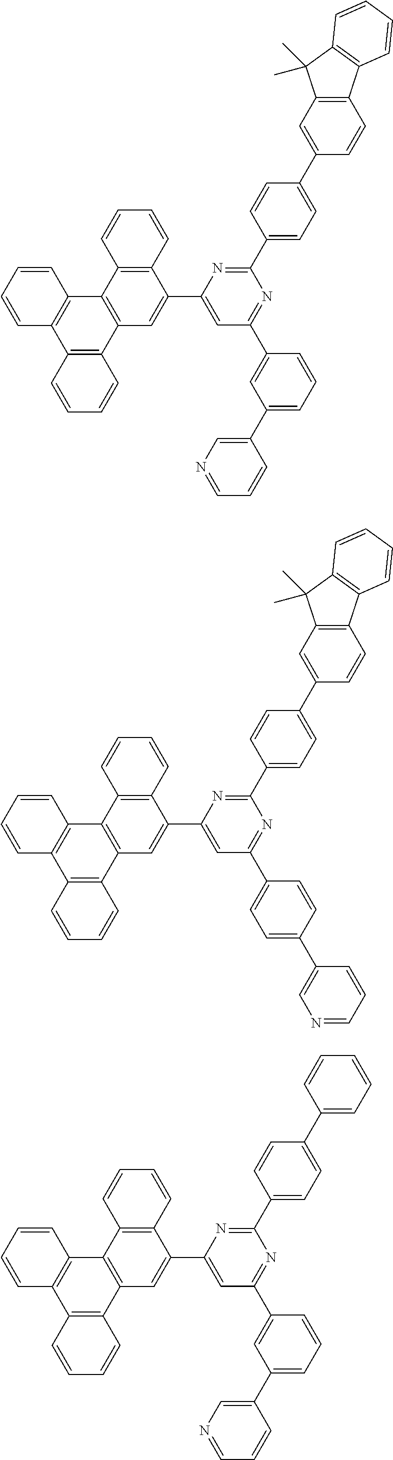

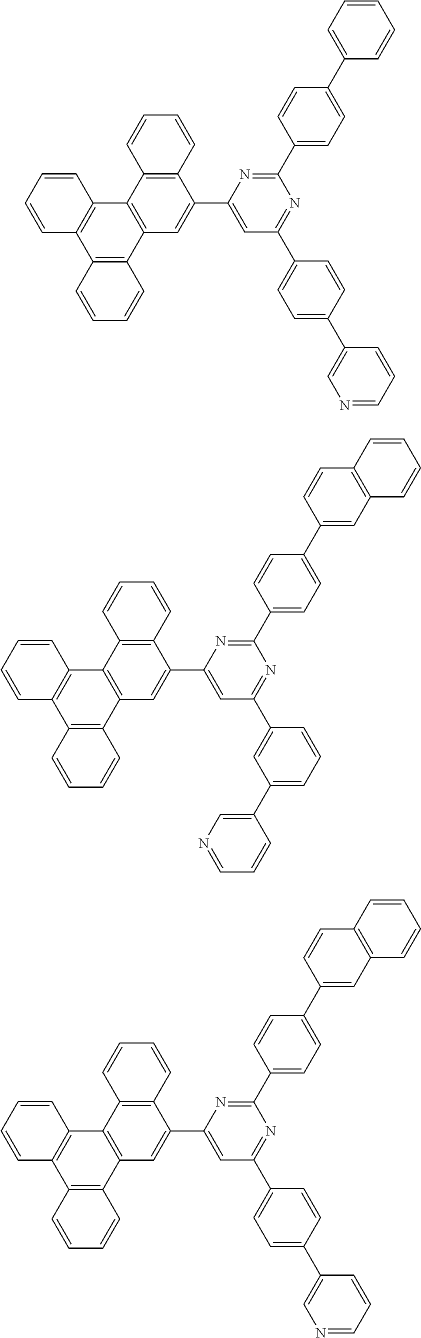

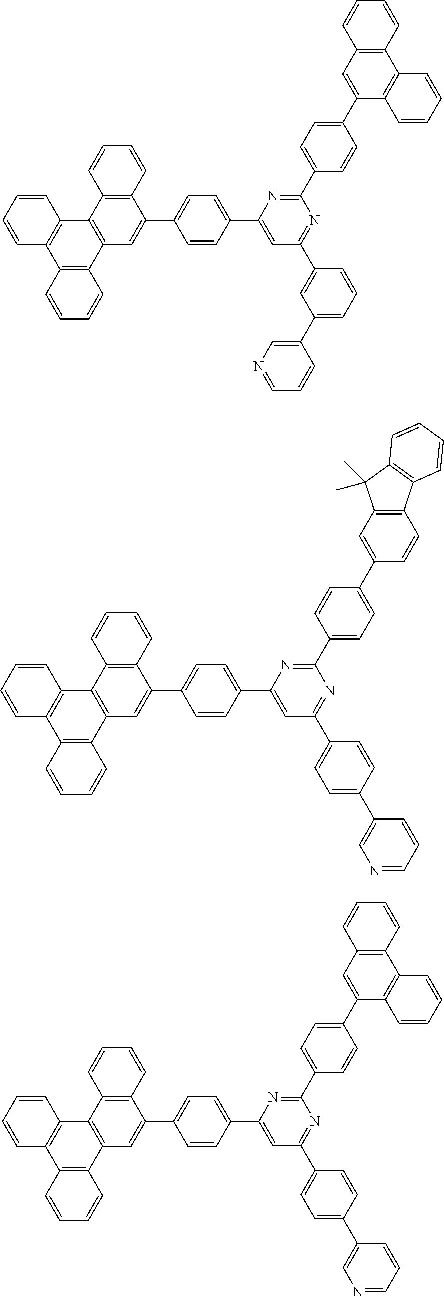

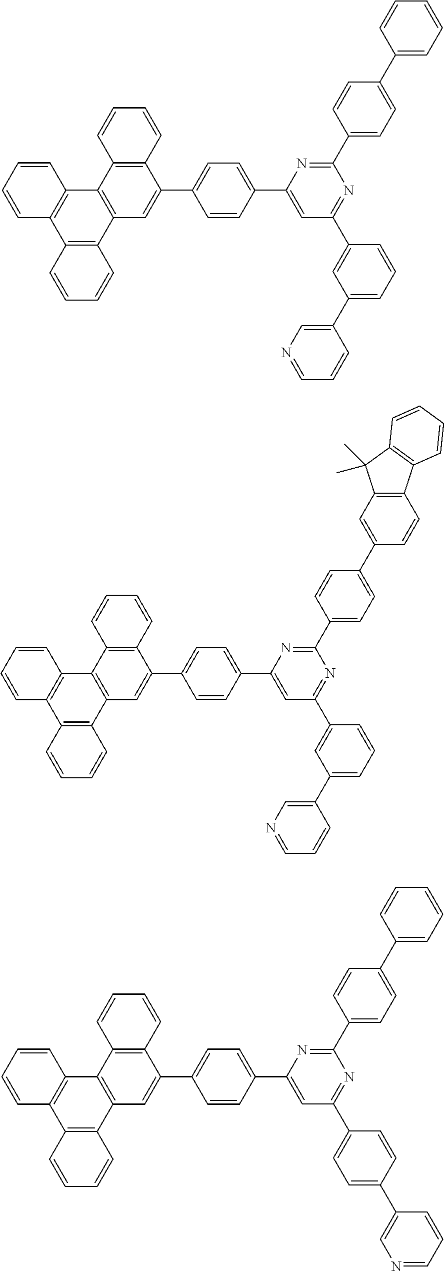 [SMILES: c1ccc(-c2ccc(-c3nc(-c4ccc(-c5cc6c7ccccc7c7ccccc7c6c6ccccc56)cc4)cc(-c4cccc(-c5cccnc5)c4)n3)cc2)cc1 ; CC1(C)c2ccccc2-c2ccc(-c3ccc(-c4nc(-c5ccc(-c6cc7c8ccccc8c8ccccc8c7c7ccccc67)cc5)cc(-c5cccc(-c6cccnc6)c5)n4)cc3)cc21 ; c1ccc(-c2ccc(-c3nc(-c4ccc(-c5cccnc5)cc4)cc(-c4ccc(-c5cc6c7ccccc7c7ccccc7c6c6ccccc56)cc4)n3)cc2)cc1]

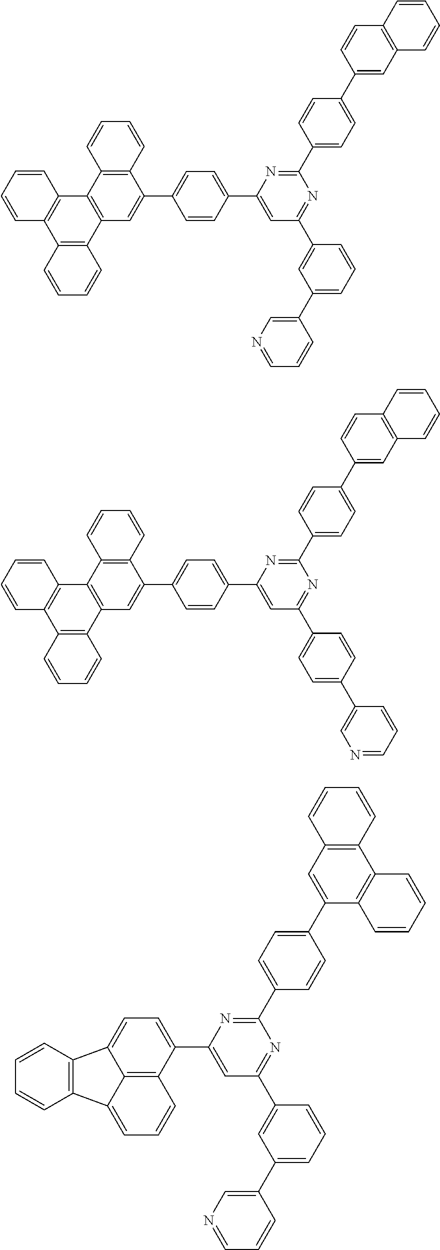

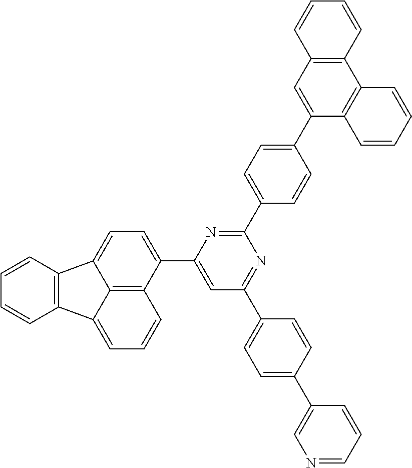 [SMILES: c1cncc(-c2ccc(-c3cc(-c4ccc5c6c(cccc46)-c4ccccc4-5)nc(-c4ccc(-c5cc6ccccc6c6ccccc56)cc4)n3)cc2)c1]

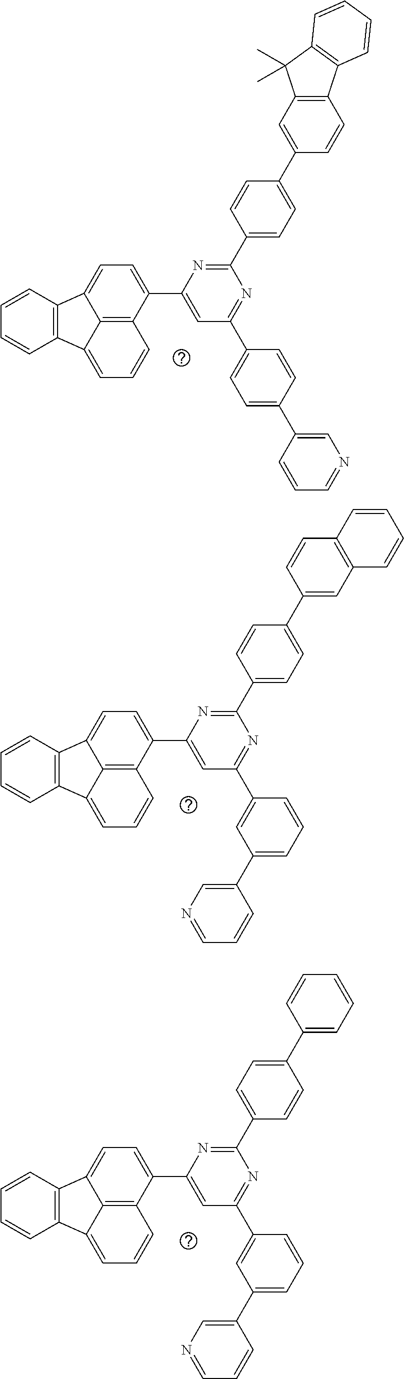

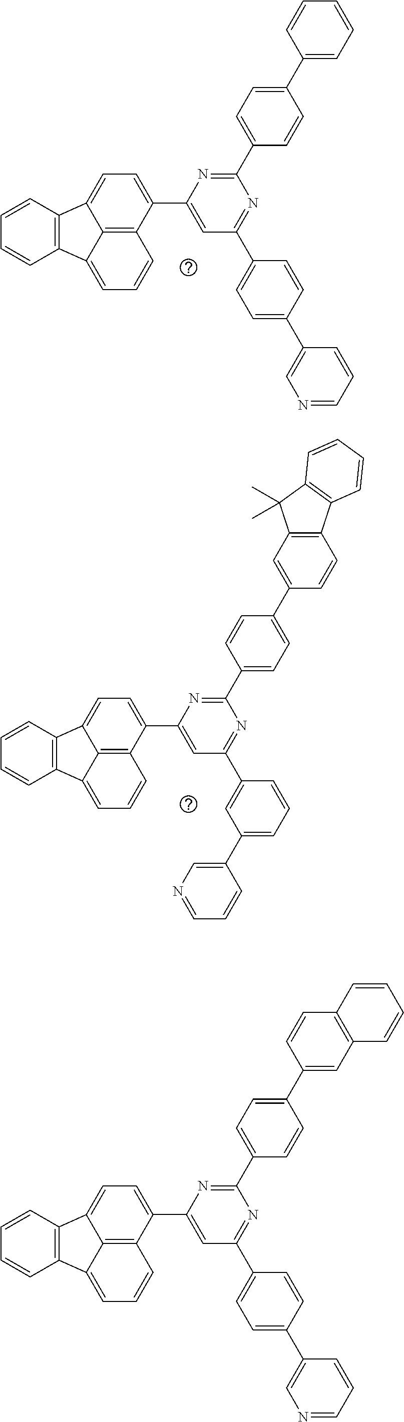

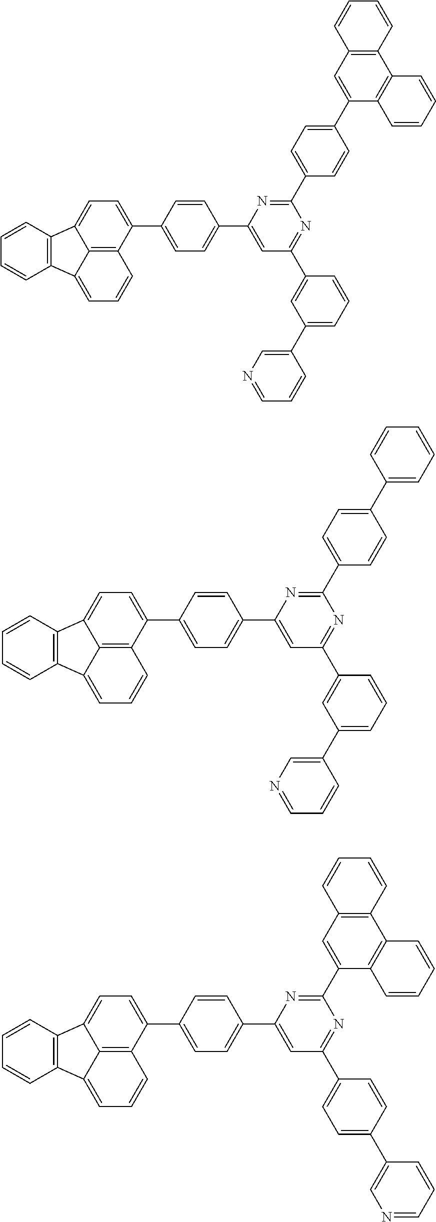 [SMILES: c1cncc(-c2cccc(-c3cc(-c4ccc(-c5ccc6c7c(cccc57)-c5ccccc5-6)cc4)nc(-c4ccc(-c5cc6ccccc6c6ccccc56)cc4)n3)c2)c1 ; c1ccc(-c2ccc(-c3nc(-c4ccc(-c5ccc6c7c(cccc57)-c5ccccc5-6)cc4)cc(-c4cccc(-c5cccnc5)c4)n3)cc2)cc1 ; c1cncc(-c2ccc(-c3cc(-c4ccc(-c5ccc6c7c(cccc57)-c5ccccc5-6)cc4)nc(-c4cc5ccccc5c5ccccc45)n3)cc2)c1]

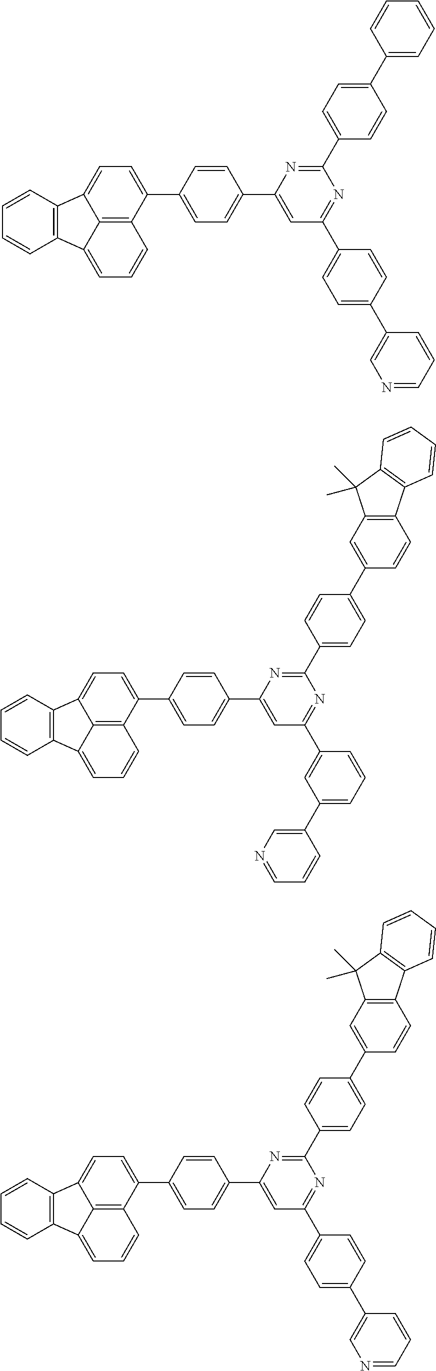

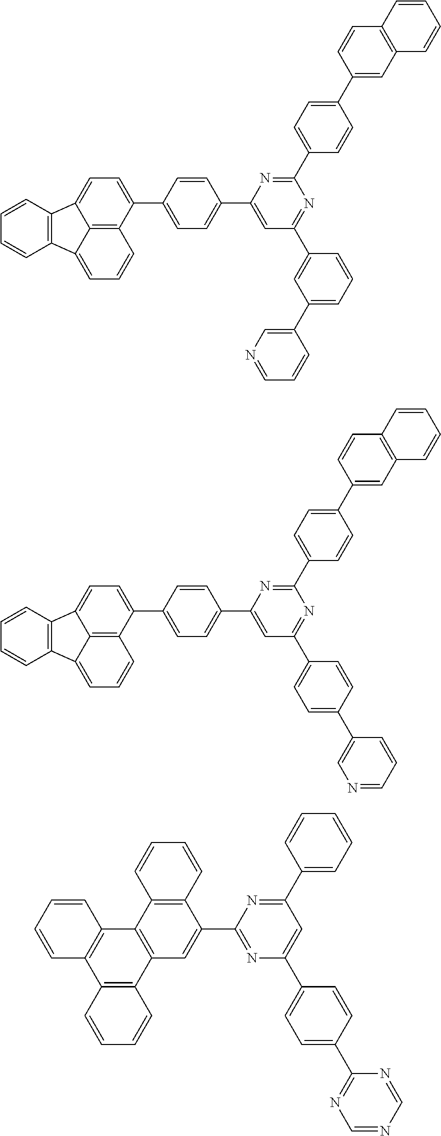

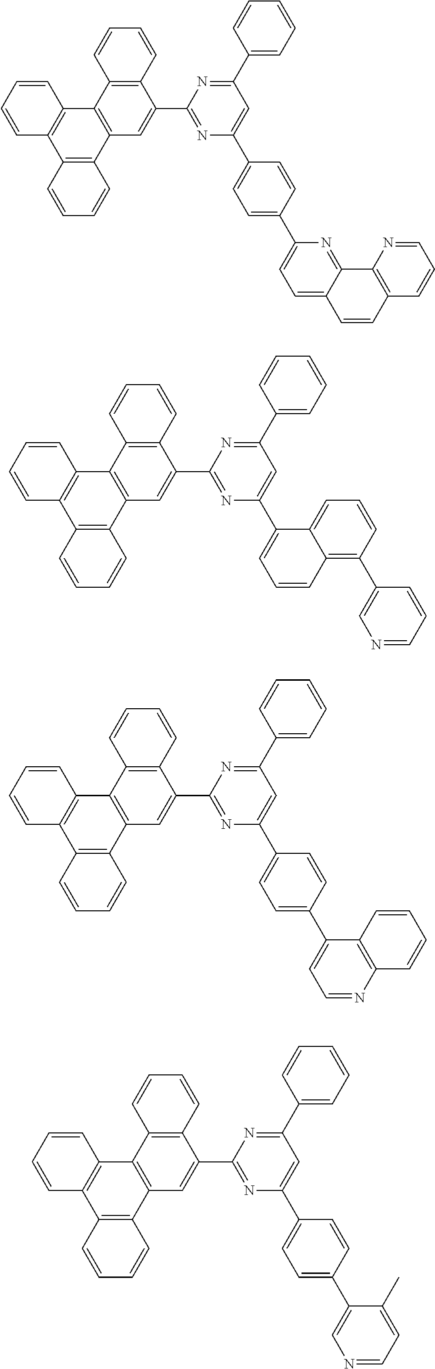

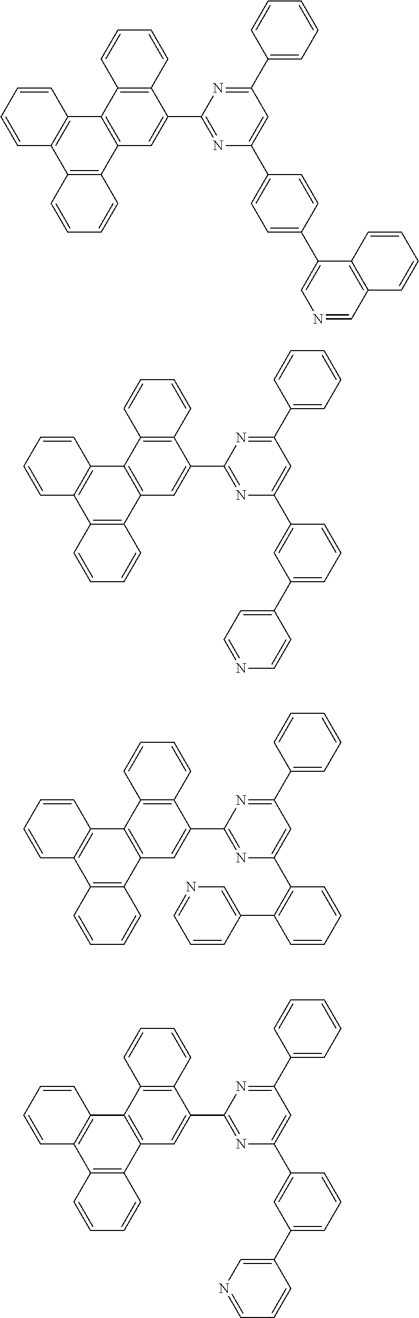

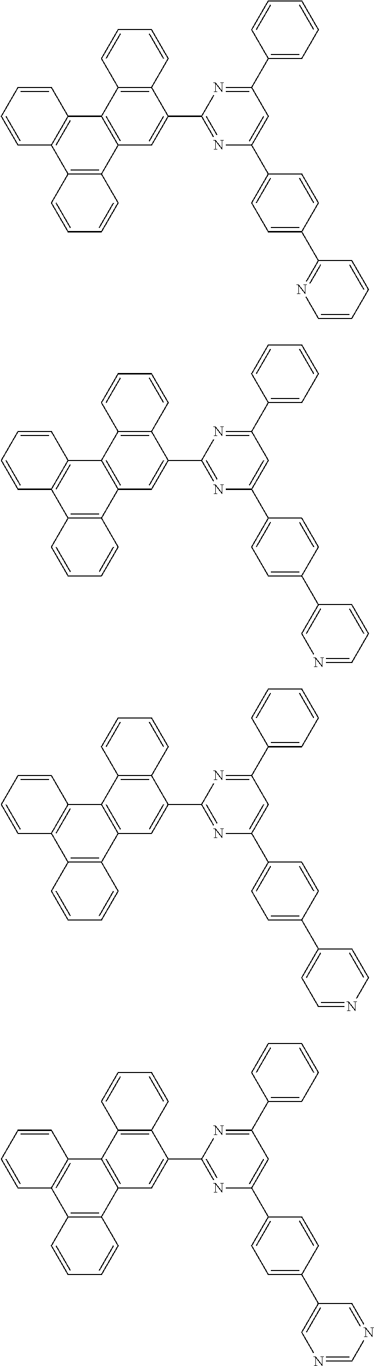

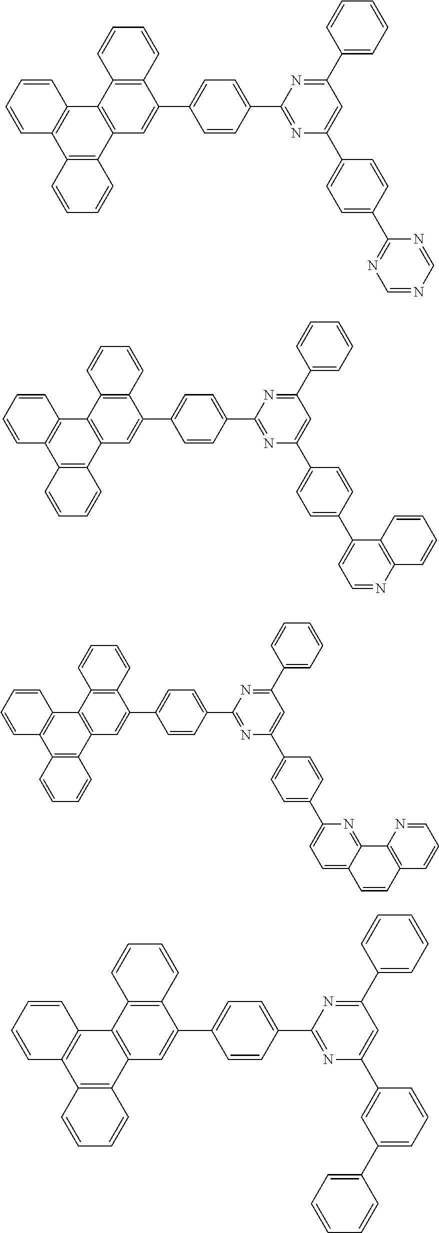

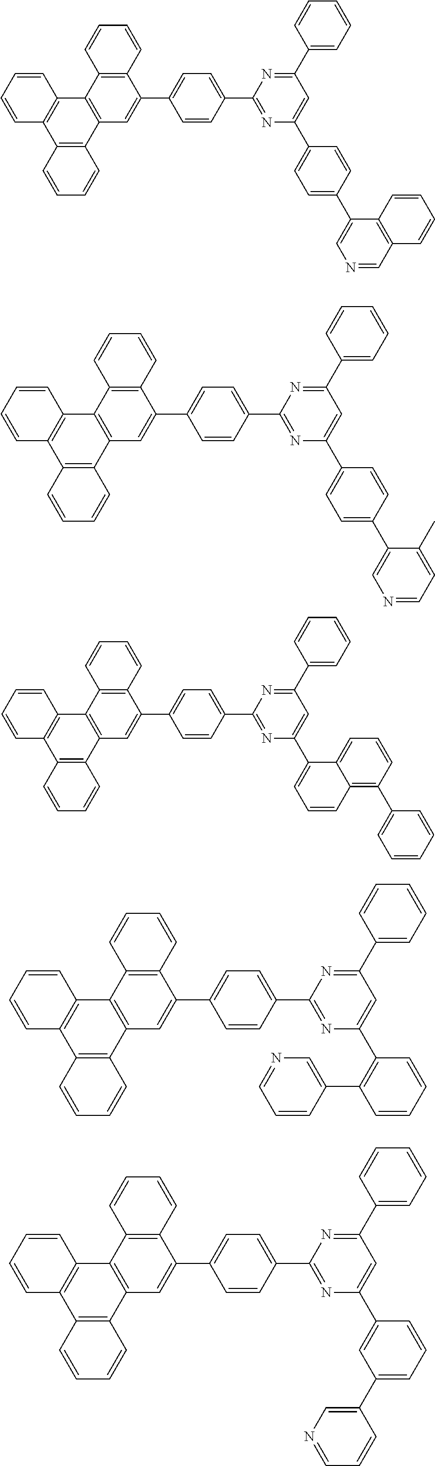

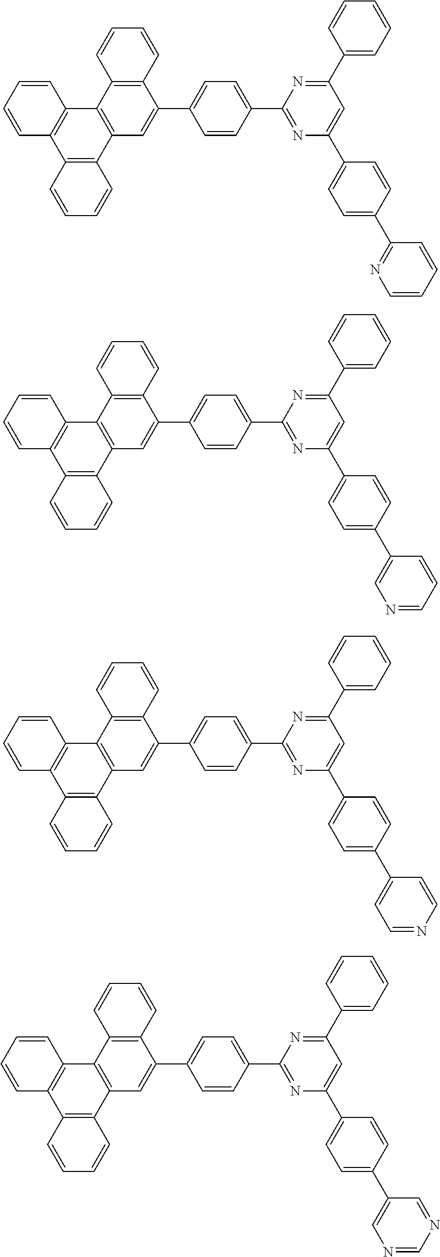

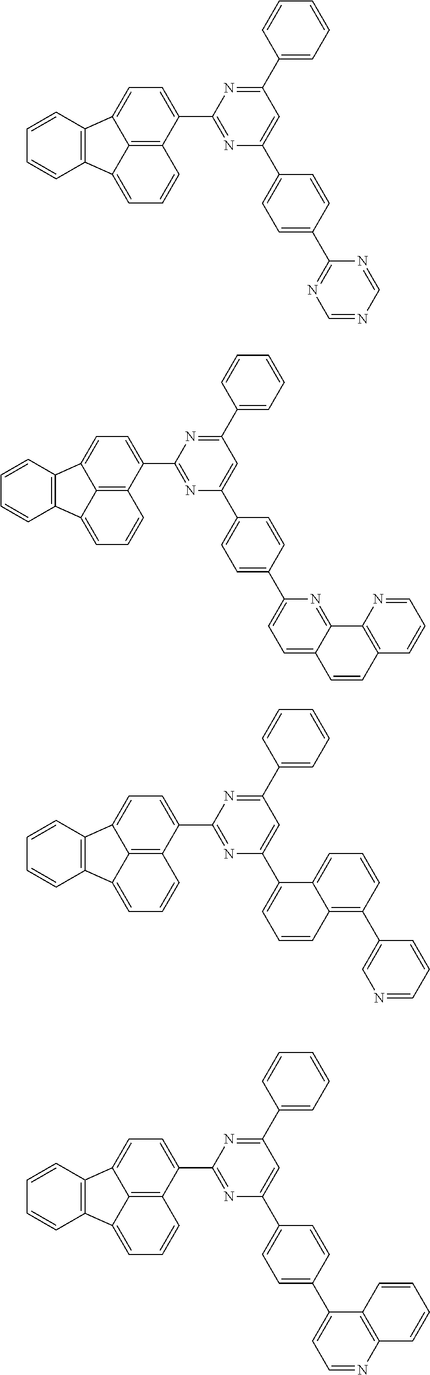

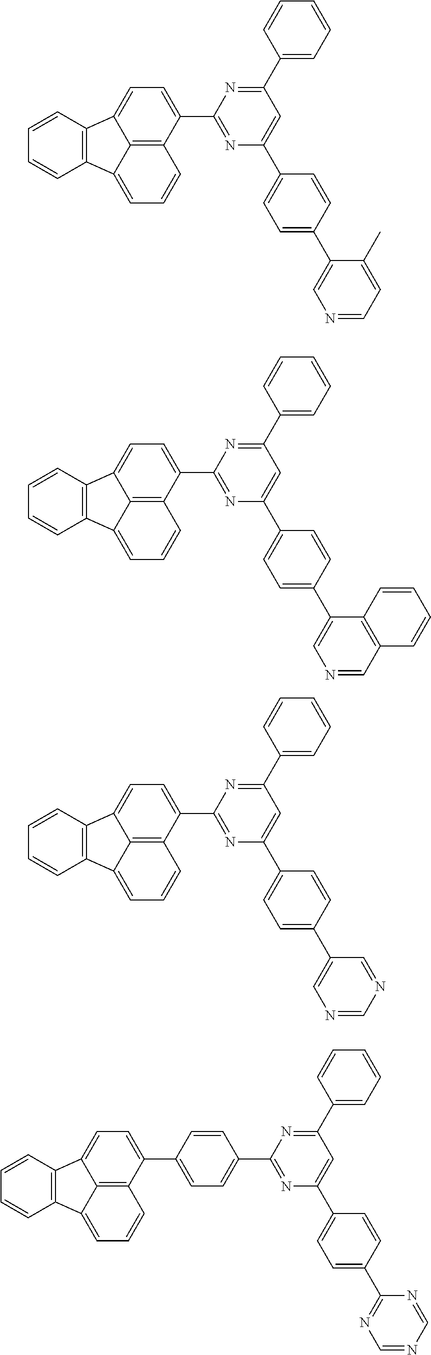

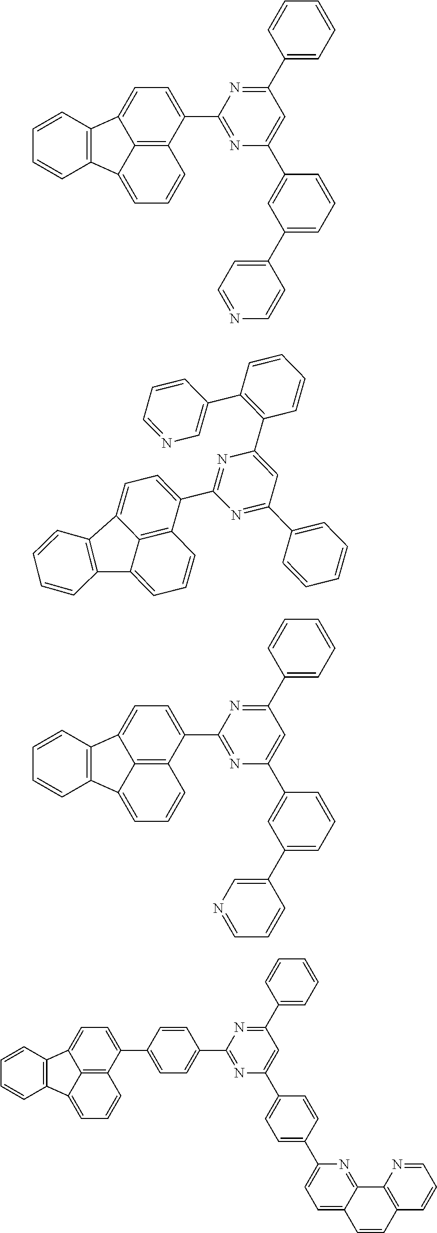

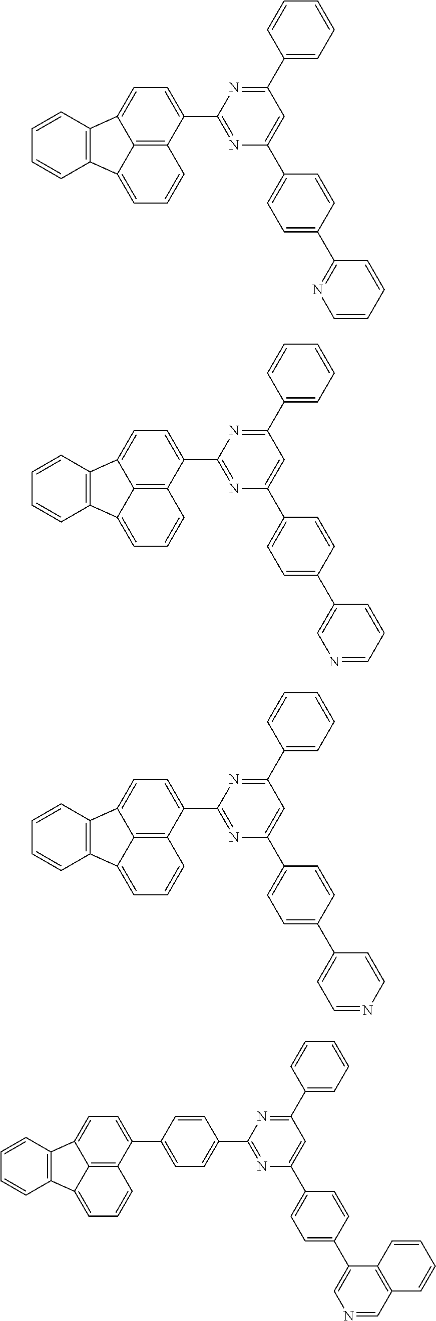

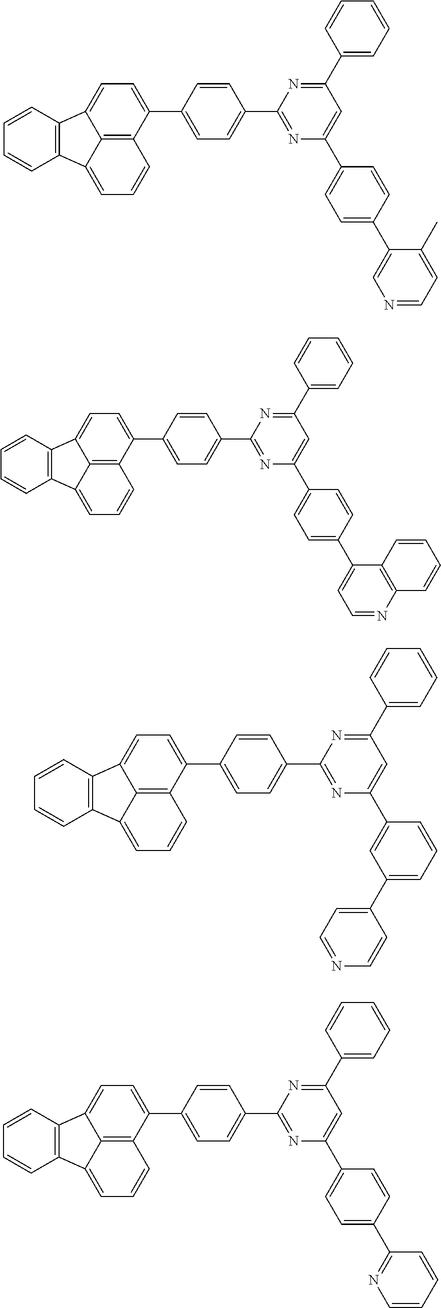

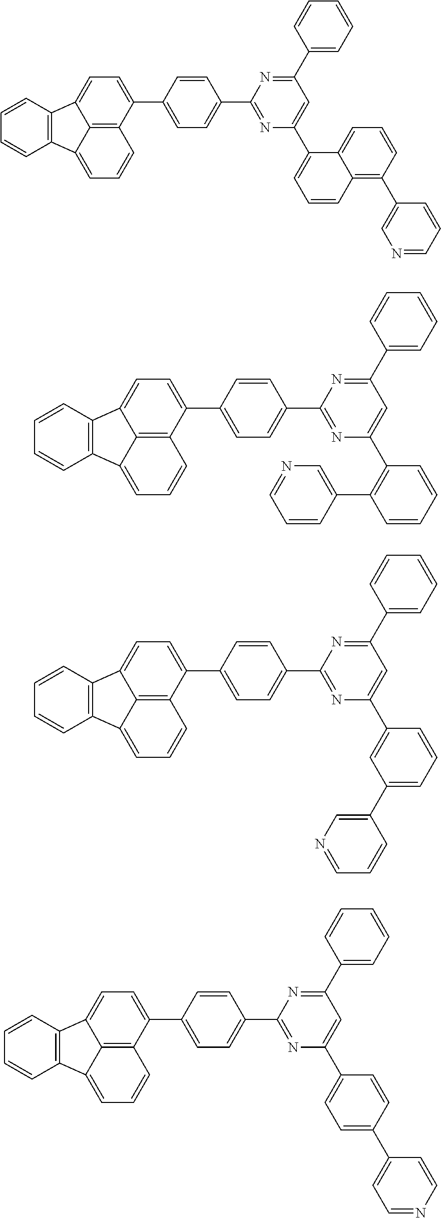

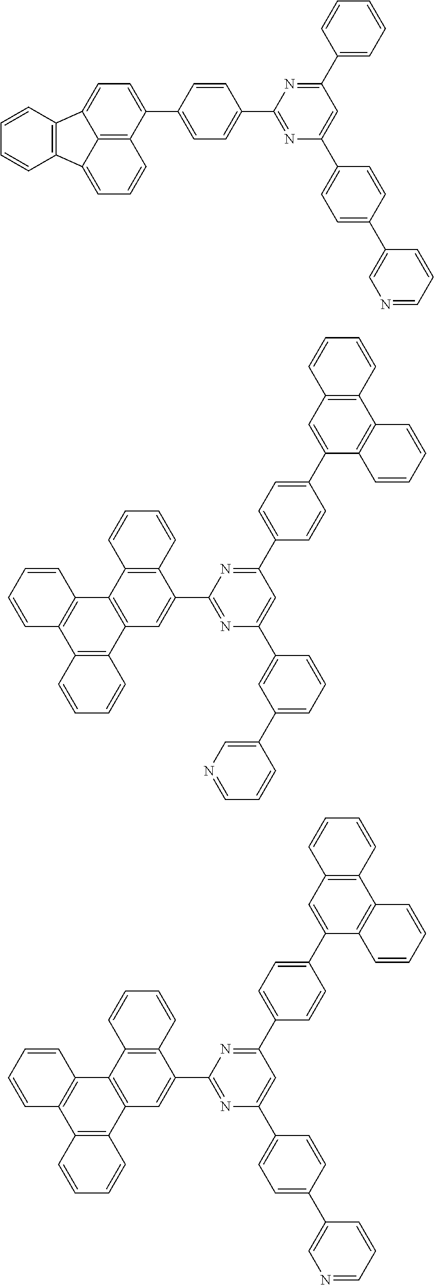

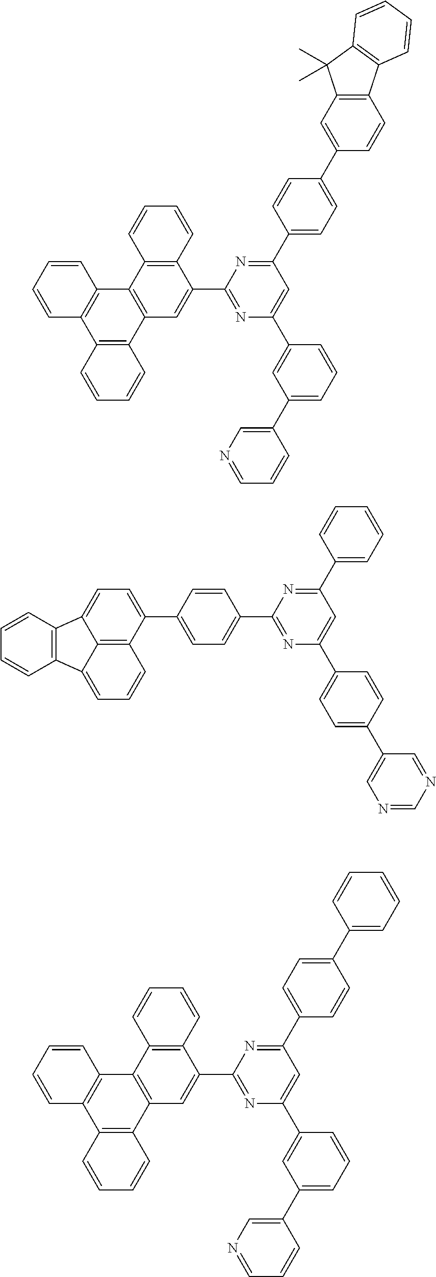 [SMILES: CC1(C)c2ccccc2-c2ccc(-c3ccc(-c4cc(-c5cccc(-c6cccnc6)c5)nc(-c5cc6c7ccccc7c7ccccc7c6c6ccccc56)n4)cc3)cc21 ; c1ccc(-c2cc(-c3ccc(-c4cncnc4)cc3)nc(-c3ccc(-c4ccc5c6c(cccc46)-c4ccccc4-5)cc3)n2)cc1 ; c1ccc(-c2ccc(-c3cc(-c4cccc(-c5cccnc5)c4)nc(-c4cc5c6ccccc6c6ccccc6c5c5ccccc45)n3)cc2)cc1]

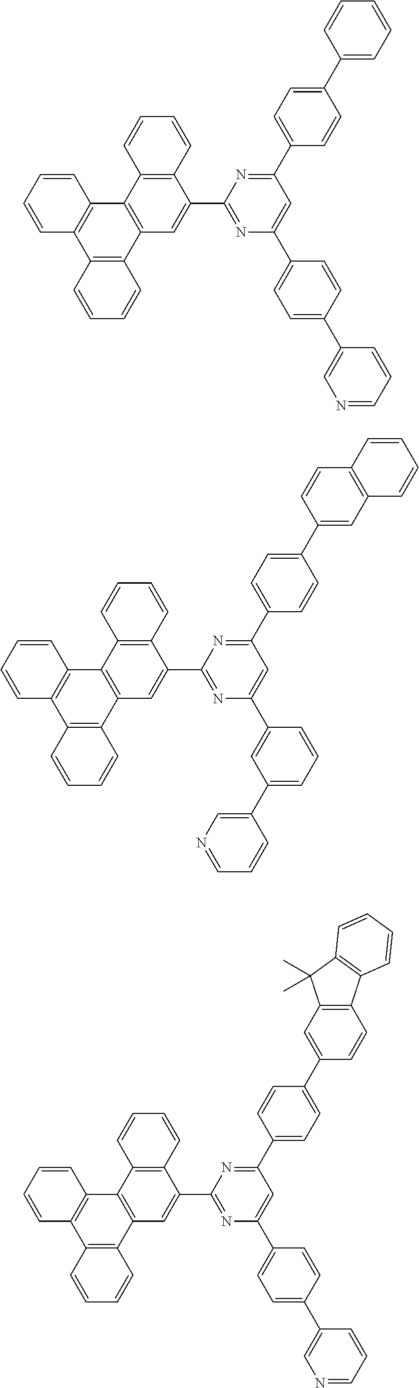

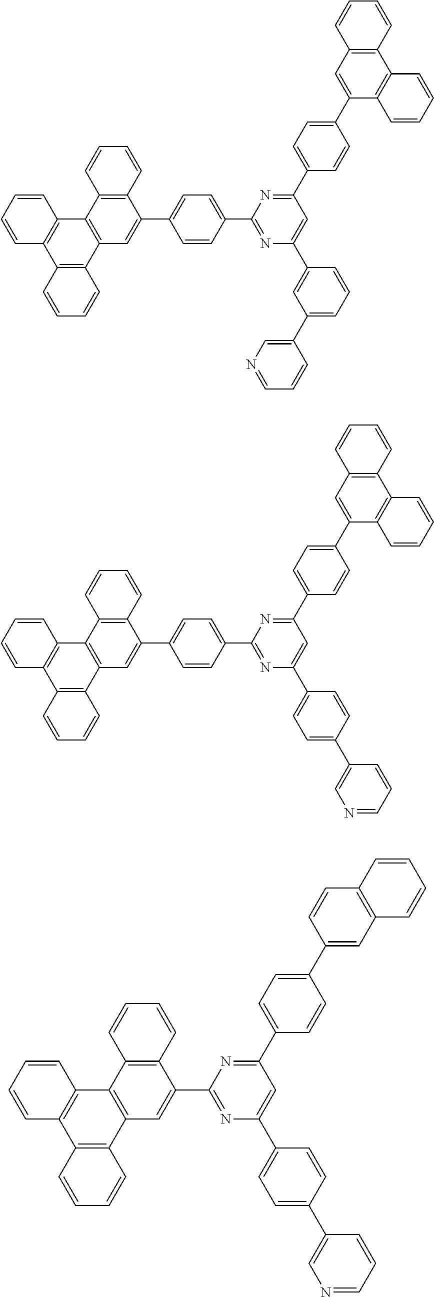 [SMILES: c1cncc(-c2cccc(-c3cc(-c4ccc(-c5cc6ccccc6c6ccccc56)cc4)nc(-c4ccc(-c5cc6c7ccccc7c7ccccc7c6c6ccccc56)cc4)n3)c2)c1 ; c1cncc(-c2ccc(-c3cc(-c4ccc(-c5cc6ccccc6c6ccccc56)cc4)nc(-c4ccc(-c5cc6c7ccccc7c7ccccc7c6c6ccccc56)cc4)n3)cc2)c1 ; c1cncc(-c2ccc(-c3cc(-c4ccc(-c5ccc6ccccc6c5)cc4)nc(-c4cc5c6ccccc6c6ccccc6c5c5ccccc45)n3)cc2)c1]

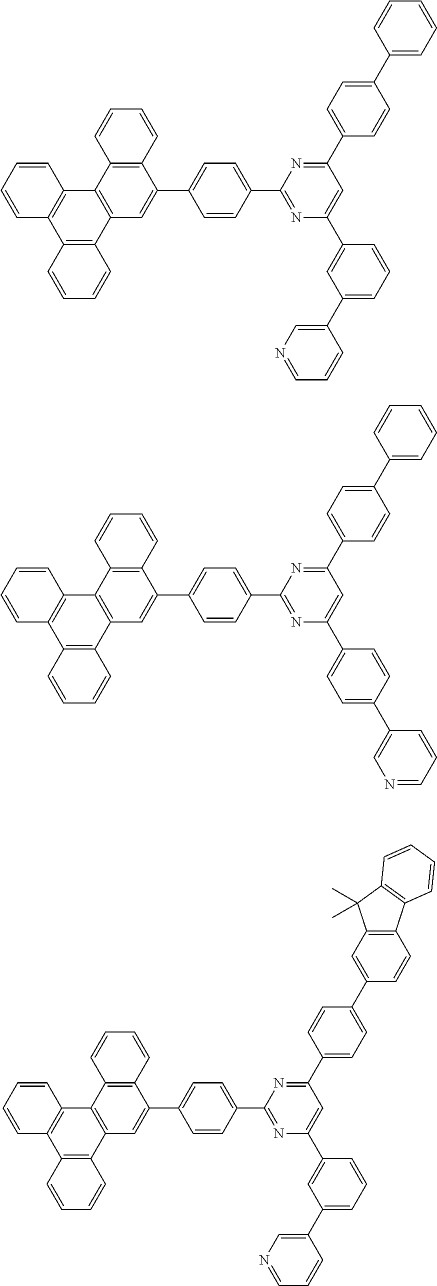 [SMILES: c1ccc(-c2ccc(-c3cc(-c4cccc(-c5cccnc5)c4)nc(-c4ccc(-c5cc6c7ccccc7c7ccccc7c6c6ccccc56)cc4)n3)cc2)cc1 ; c1ccc(-c2ccc(-c3cc(-c4ccc(-c5cccnc5)cc4)nc(-c4ccc(-c5cc6c7ccccc7c7ccccc7c6c6ccccc56)cc4)n3)cc2)cc1 ; CC1(C)c2ccccc2-c2ccc(-c3ccc(-c4cc(-c5cccc(-c6cccnc6)c5)nc(-c5ccc(-c6cc7c8ccccc8c8ccccc8c7c7ccccc67)cc5)n4)cc3)cc21]

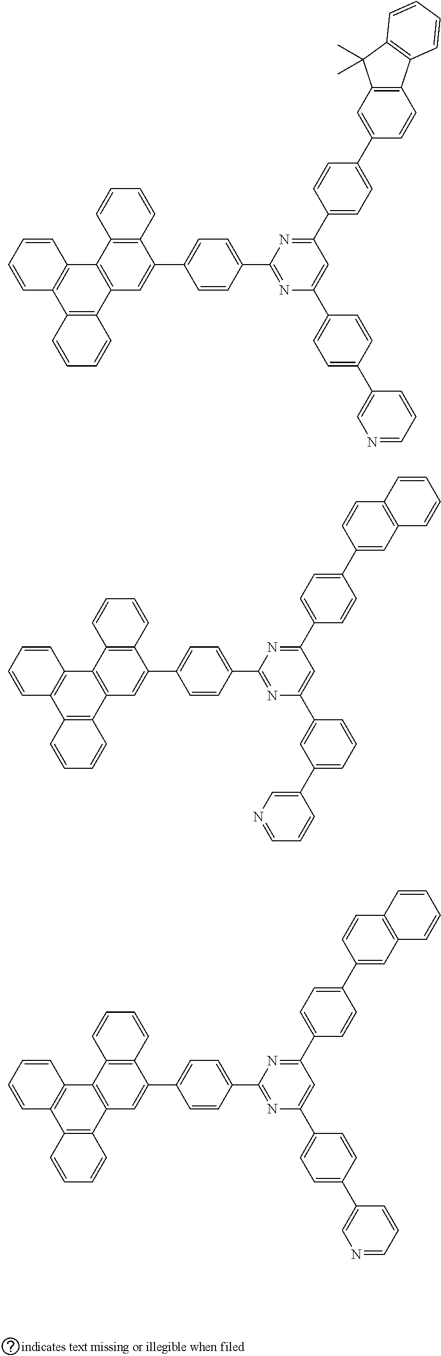

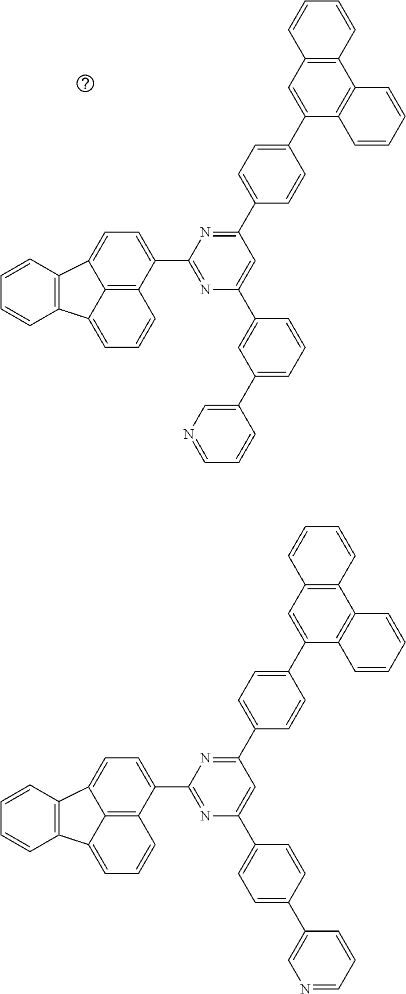 [SMILES: c1cncc(-c2cccc(-c3cc(-c4ccc(-c5cc6ccccc6c6ccccc56)cc4)nc(-c4ccc5c6c(cccc46)-c4ccccc4-5)n3)c2)c1 ; c1cncc(-c2ccc(-c3cc(-c4ccc(-c5cc6ccccc6c6ccccc56)cc4)nc(-c4ccc5c6c(cccc46)-c4ccccc4-5)n3)cc2)c1]

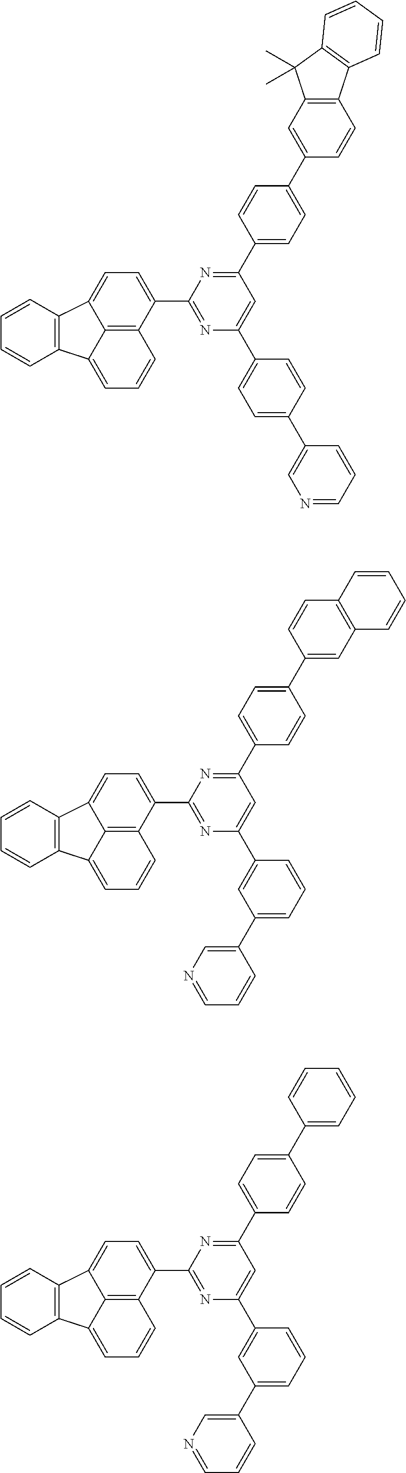 [SMILES: CC1(C)c2ccccc2-c2ccc(-c3ccc(-c4cc(-c5ccc(-c6cccnc6)cc5)nc(-c5ccc6c7c(cccc57)-c5ccccc5-6)n4)cc3)cc21 ; c1cncc(-c2cccc(-c3cc(-c4ccc(-c5ccc6ccccc6c5)cc4)nc(-c4ccc5c6c(cccc46)-c4ccccc4-5)n3)c2)c1 ; c1ccc(-c2ccc(-c3cc(-c4cccc(-c5cccnc5)c4)nc(-c4ccc5c6c(cccc46)-c4ccccc4-5)n3)cc2)cc1]

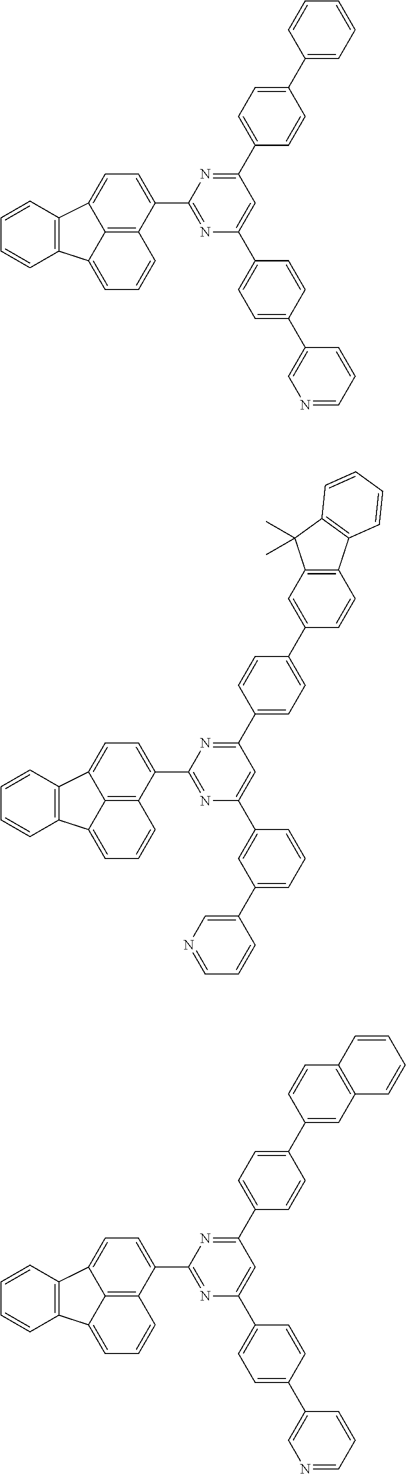 [SMILES: c1ccc(-c2ccc(-c3cc(-c4ccc(-c5cccnc5)cc4)nc(-c4ccc5c6c(cccc46)-c4ccccc4-5)n3)cc2)cc1 ; CC1(C)c2ccccc2-c2ccc(-c3ccc(-c4cc(-c5cccc(-c6cccnc6)c5)nc(-c5ccc6c7c(cccc57)-c5ccccc5-6)n4)cc3)cc21 ; c1cncc(-c2ccc(-c3cc(-c4ccc(-c5ccc6ccccc6c5)cc4)nc(-c4ccc5c6c(cccc46)-c4ccccc4-5)n3)cc2)c1]

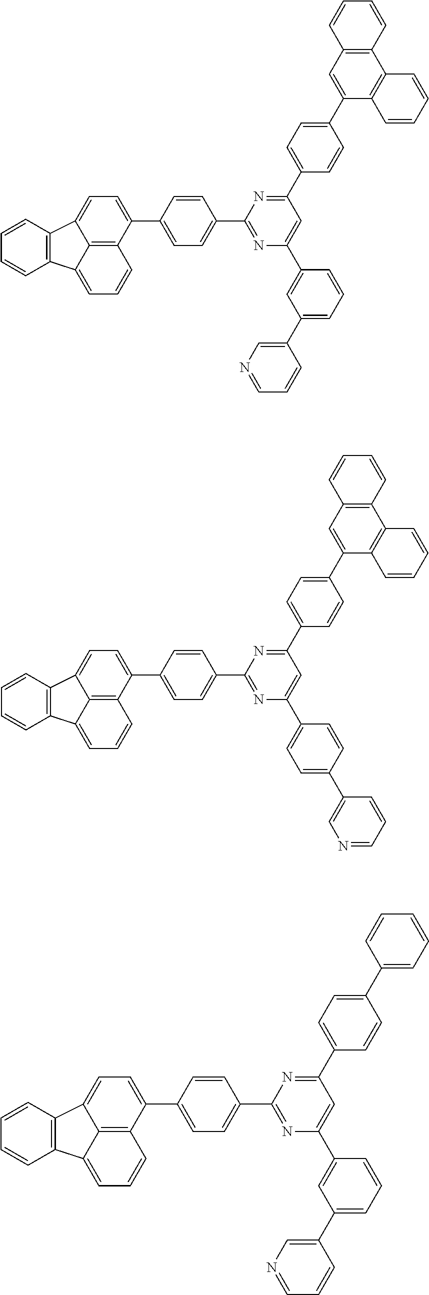 [SMILES: c1cncc(-c2cccc(-c3cc(-c4ccc(-c5cc6ccccc6c6ccccc56)cc4)nc(-c4ccc(-c5ccc6c7c(cccc57)-c5ccccc5-6)cc4)n3)c2)c1 ; c1cncc(-c2ccc(-c3cc(-c4ccc(-c5cc6ccccc6c6ccccc56)cc4)nc(-c4ccc(-c5ccc6c7c(cccc57)-c5ccccc5-6)cc4)n3)cc2)c1 ; c1ccc(-c2ccc(-c3cc(-c4cccc(-c5cccnc5)c4)nc(-c4ccc(-c5ccc6c7c(cccc57)-c5ccccc5-6)cc4)n3)cc2)cc1]

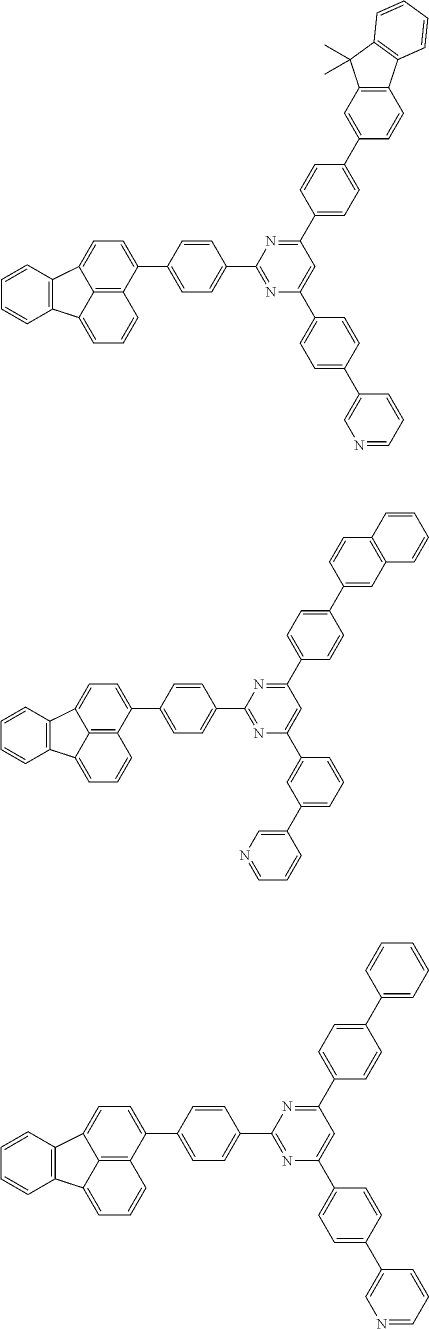 [SMILES: CC1(C)c2ccccc2-c2ccc(-c3ccc(-c4cc(-c5ccc(-c6cccnc6)cc5)nc(-c5ccc(-c6ccc7c8c(cccc68)-c6ccccc6-7)cc5)n4)cc3)cc21 ; c1cncc(-c2cccc(-c3cc(-c4ccc(-c5ccc6ccccc6c5)cc4)nc(-c4ccc(-c5ccc6c7c(cccc57)-c5ccccc5-6)cc4)n3)c2)c1 ; c1ccc(-c2ccc(-c3cc(-c4ccc(-c5cccnc5)cc4)nc(-c4ccc(-c5ccc6c7c(cccc57)-c5ccccc5-6)cc4)n3)cc2)cc1]

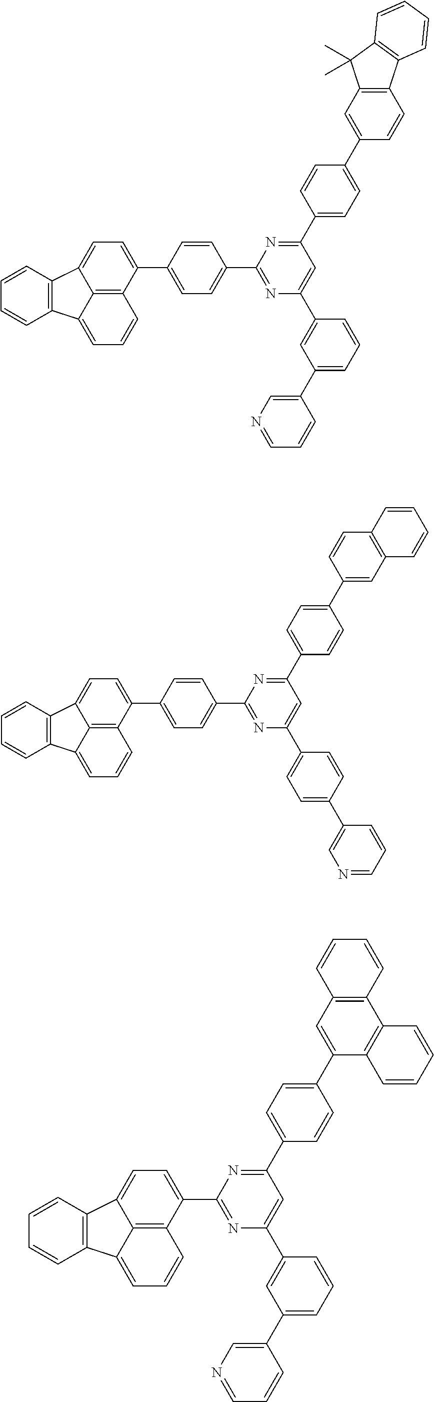 [SMILES: CC1(C)c2ccccc2-c2ccc(-c3ccc(-c4cc(-c5cccc(-c6cccnc6)c5)nc(-c5ccc(-c6ccc7c8c(cccc68)-c6ccccc6-7)cc5)n4)cc3)cc21 ; c1cncc(-c2ccc(-c3cc(-c4ccc(-c5ccc6ccccc6c5)cc4)nc(-c4ccc(-c5ccc6c7c(cccc57)-c5ccccc5-6)cc4)n3)cc2)c1 ; c1cncc(-c2cccc(-c3cc(-c4ccc(-c5cc6ccccc6c6ccccc56)cc4)nc(-c4ccc5c6c(cccc46)-c4ccccc4-5)n3)c2)c1]

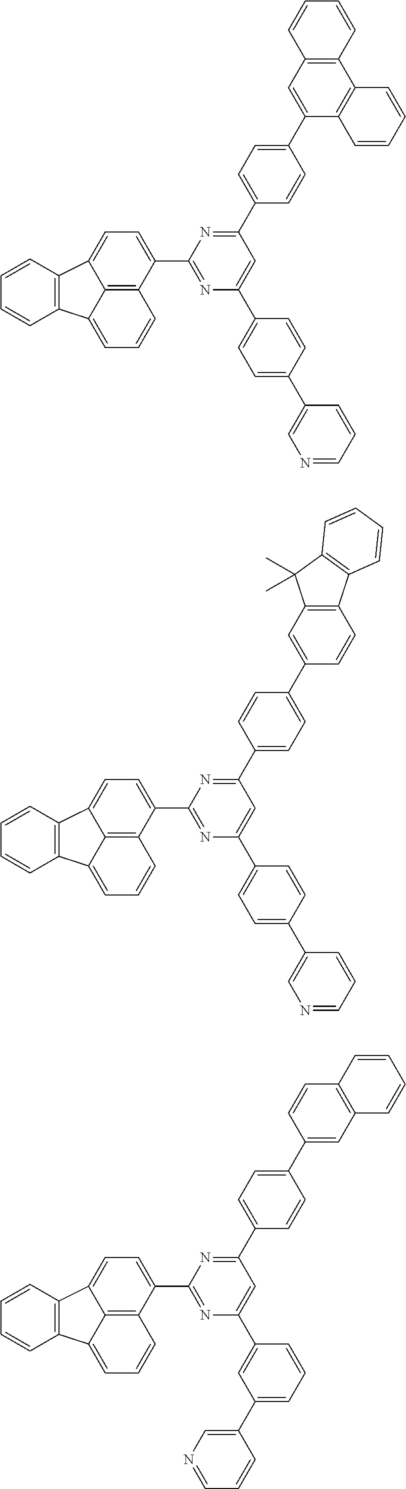 [SMILES: c1cncc(-c2ccc(-c3cc(-c4ccc(-c5cc6ccccc6c6ccccc56)cc4)nc(-c4ccc5c6c(cccc46)-c4ccccc4-5)n3)cc2)c1 ; CC1(C)c2ccccc2-c2ccc(-c3ccc(-c4cc(-c5ccc(-c6cccnc6)cc5)nc(-c5ccc6c7c(cccc57)-c5ccccc5-6)n4)cc3)cc21 ; c1cncc(-c2cccc(-c3cc(-c4ccc(-c5ccc6ccccc6c5)cc4)nc(-c4ccc5c6c(cccc46)-c4ccccc4-5)n3)c2)c1]

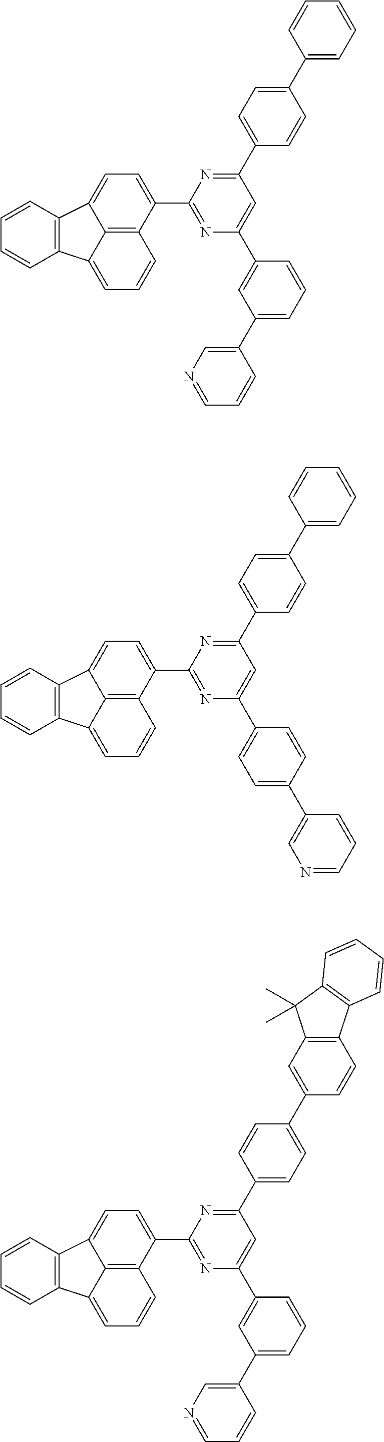 [SMILES: c1ccc(-c2ccc(-c3cc(-c4cccc(-c5cccnc5)c4)nc(-c4ccc5c6c(cccc46)-c4ccccc4-5)n3)cc2)cc1 ; c1ccc(-c2ccc(-c3cc(-c4ccc(-c5cccnc5)cc4)nc(-c4ccc5c6c(cccc46)-c4ccccc4-5)n3)cc2)cc1 ; CC1(C)c2ccccc2-c2ccc(-c3ccc(-c4cc(-c5cccc(-c6cccnc6)c5)nc(-c5ccc6c7c(cccc57)-c5ccccc5-6)n4)cc3)cc21]

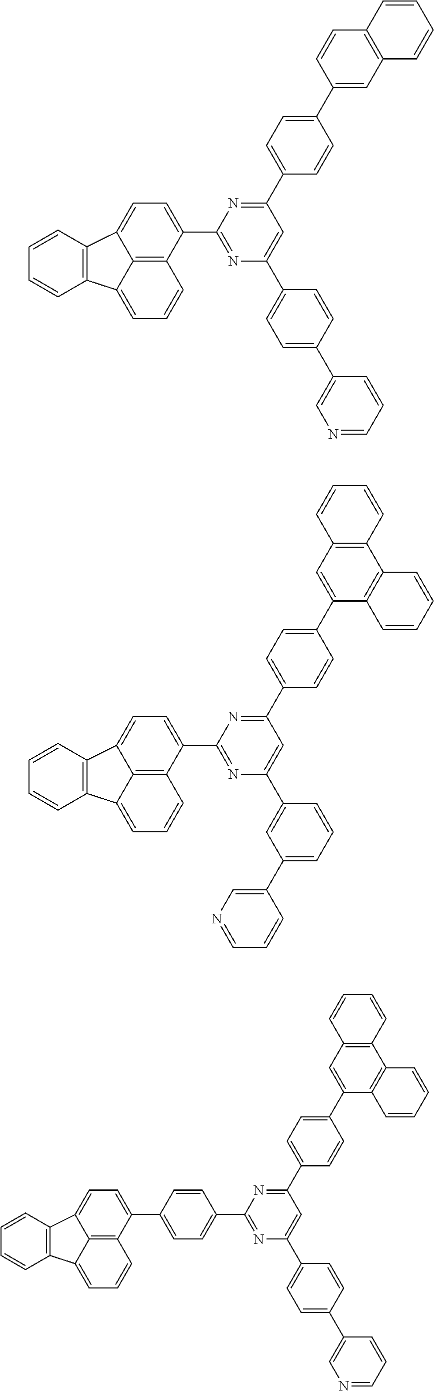 [SMILES: c1cncc(-c2ccc(-c3cc(-c4ccc(-c5ccc6ccccc6c5)cc4)nc(-c4ccc5c6c(cccc46)-c4ccccc4-5)n3)cc2)c1 ; c1cncc(-c2cccc(-c3cc(-c4ccc(-c5cc6ccccc6c6ccccc56)cc4)nc(-c4ccc5c6c(cccc46)-c4ccccc4-5)n3)c2)c1 ; c1cncc(-c2ccc(-c3cc(-c4ccc(-c5cc6ccccc6c6ccccc56)cc4)nc(-c4ccc(-c5ccc6c7c(cccc57)-c5ccccc5-6)cc4)n3)cc2)c1]

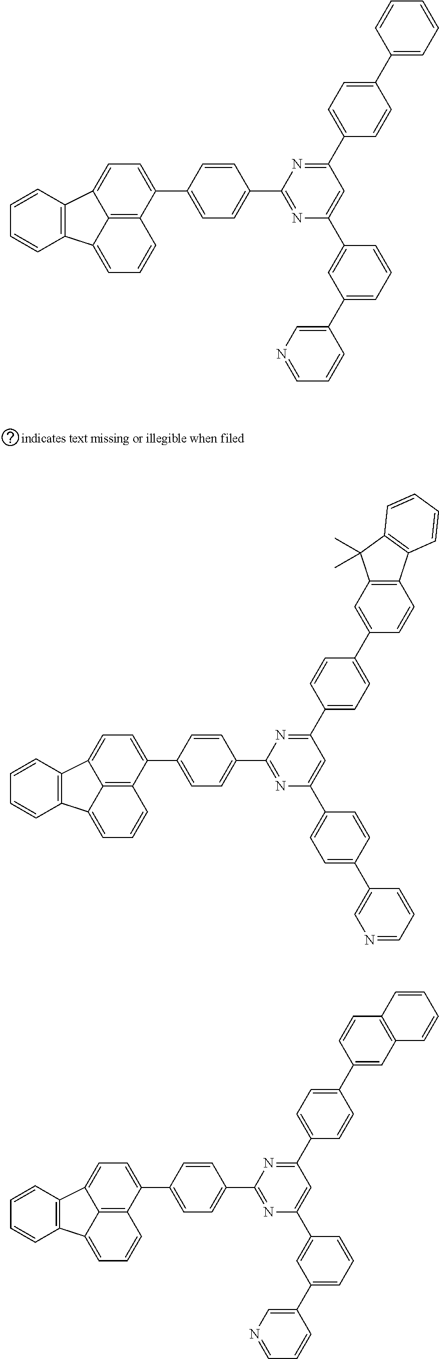 [SMILES: c1ccc(-c2ccc(-c3cc(-c4cccc(-c5cccnc5)c4)nc(-c4ccc(-c5ccc6c7c(cccc57)-c5ccccc5-6)cc4)n3)cc2)cc1 ; CC1(C)c2ccccc2-c2ccc(-c3ccc(-c4cc(-c5ccc(-c6cccnc6)cc5)nc(-c5ccc(-c6ccc7c8c(cccc68)-c6ccccc6-7)cc5)n4)cc3)cc21 ; c1cncc(-c2cccc(-c3cc(-c4ccc(-c5ccc6ccccc6c5)cc4)nc(-c4ccc(-c5ccc6c7c(cccc57)-c5ccccc5-6)cc4)n3)c2)c1]

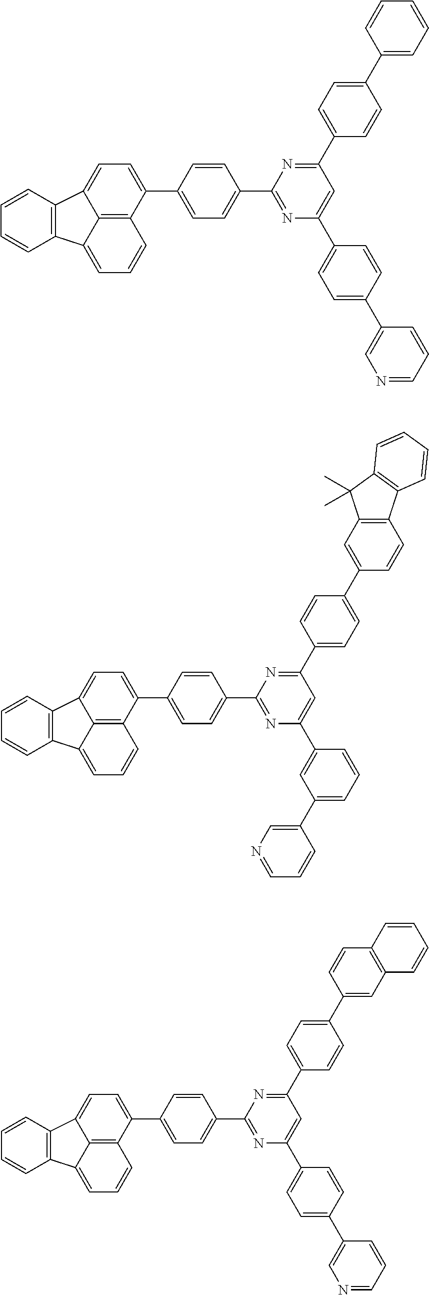 [SMILES: c1ccc(-c2ccc(-c3cc(-c4ccc(-c5cccnc5)cc4)nc(-c4ccc(-c5ccc6c7c(cccc57)-c5ccccc5-6)cc4)n3)cc2)cc1 ; CC1(C)c2ccccc2-c2ccc(-c3ccc(-c4cc(-c5cccc(-c6cccnc6)c5)nc(-c5ccc(-c6ccc7c8c(cccc68)-c6ccccc6-7)cc5)n4)cc3)cc21 ; c1cncc(-c2ccc(-c3cc(-c4ccc(-c5ccc6ccccc6c5)cc4)nc(-c4ccc(-c5ccc6c7c(cccc57)-c5ccccc5-6)cc4)n3)cc2)c1]

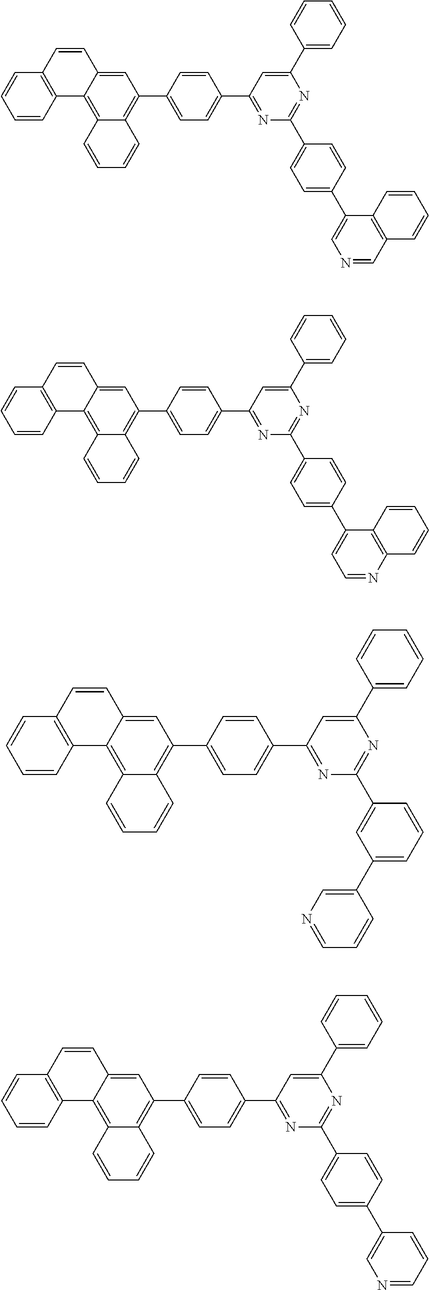 [SMILES: c1ccc(-c2cc(-c3ccc(-c4cc5ccc6ccccc6c5c5ccccc45)cc3)nc(-c3ccc(-c4cncc5ccccc45)cc3)n2)cc1 ; c1ccc(-c2cc(-c3ccc(-c4cc5ccc6ccccc6c5c5ccccc45)cc3)nc(-c3ccc(-c4ccnc5ccccc45)cc3)n2)cc1 ; c1ccc(-c2cc(-c3ccc(-c4cc5ccc6ccccc6c5c5ccccc45)cc3)nc(-c3cccc(-c4cccnc4)c3)n2)cc1 ; c1ccc(-c2cc(-c3ccc(-c4cc5ccc6ccccc6c5c5ccccc45)cc3)nc(-c3ccc(-c4cccnc4)cc3)n2)cc1]

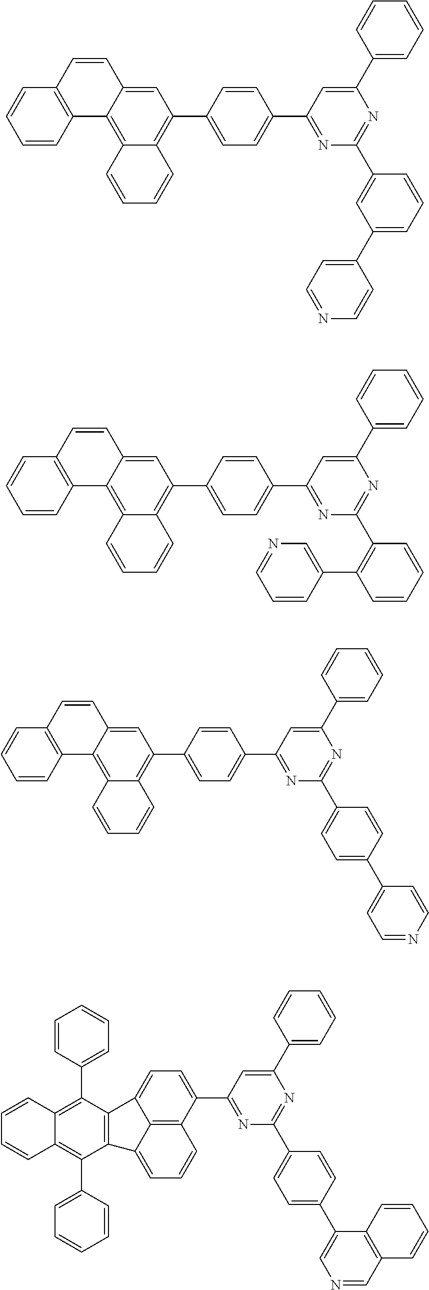 [SMILES: c1ccc(-c2cc(-c3ccc(-c4cc5ccc6ccccc6c5c5ccccc45)cc3)nc(-c3cccc(-c4ccncc4)c3)n2)cc1 ; c1ccc(-c2cc(-c3ccc(-c4cc5ccc6ccccc6c5c5ccccc45)cc3)nc(-c3ccccc3-c3cccnc3)n2)cc1 ; c1ccc(-c2cc(-c3ccc(-c4cc5ccc6ccccc6c5c5ccccc45)cc3)nc(-c3ccc(-c4ccncc4)cc3)n2)cc1 ; c1ccc(-c2cc(-c3ccc4c5c(cccc35)-c3c-4c(-c4ccccc4)c4ccccc4c3-c3ccccc3)nc(-c3ccc(-c4cncc5ccccc45)cc3)n2)cc1]

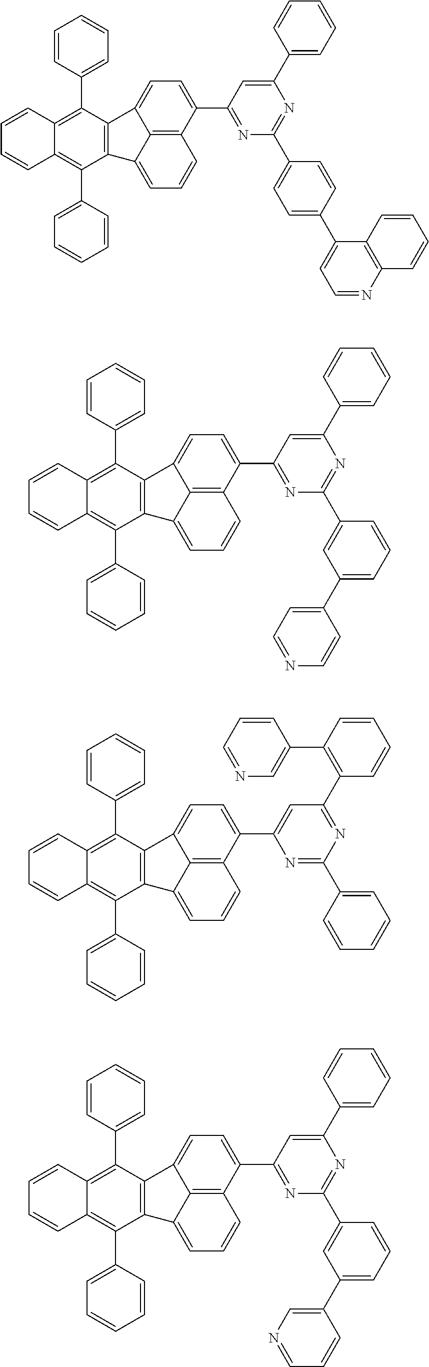 [SMILES: c1ccc(-c2cc(-c3ccc4c5c(cccc35)-c3c-4c(-c4ccccc4)c4ccccc4c3-c3ccccc3)nc(-c3ccc(-c4ccnc5ccccc45)cc3)n2)cc1 ; c1ccc(-c2cc(-c3ccc4c5c(cccc35)-c3c-4c(-c4ccccc4)c4ccccc4c3-c3ccccc3)nc(-c3cccc(-c4ccncc4)c3)n2)cc1 ; c1ccc(-c2nc(-c3ccccc3-c3cccnc3)cc(-c3ccc4c5c(cccc35)-c3c-4c(-c4ccccc4)c4ccccc4c3-c3ccccc3)n2)cc1 ; c1ccc(-c2cc(-c3ccc4c5c(cccc35)-c3c-4c(-c4ccccc4)c4ccccc4c3-c3ccccc3)nc(-c3cccc(-c4cccnc4)c3)n2)cc1]

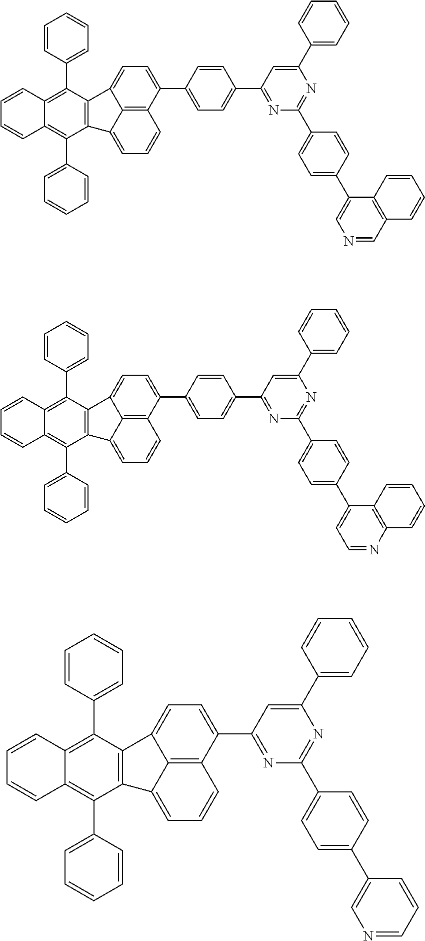 [SMILES: c1ccc(-c2cc(-c3ccc(-c4ccc5c6c(cccc46)-c4c-5c(-c5ccccc5)c5ccccc5c4-c4ccccc4)cc3)nc(-c3ccc(-c4cncc5ccccc45)cc3)n2)cc1 ; c1ccc(-c2cc(-c3ccc(-c4ccc5c6c(cccc46)-c4c-5c(-c5ccccc5)c5ccccc5c4-c4ccccc4)cc3)nc(-c3ccc(-c4ccnc5ccccc45)cc3)n2)cc1 ; c1ccc(-c2cc(-c3ccc4c5c(cccc35)-c3c-4c(-c4ccccc4)c4ccccc4c3-c3ccccc3)nc(-c3ccc(-c4cccnc4)cc3)n2)cc1]

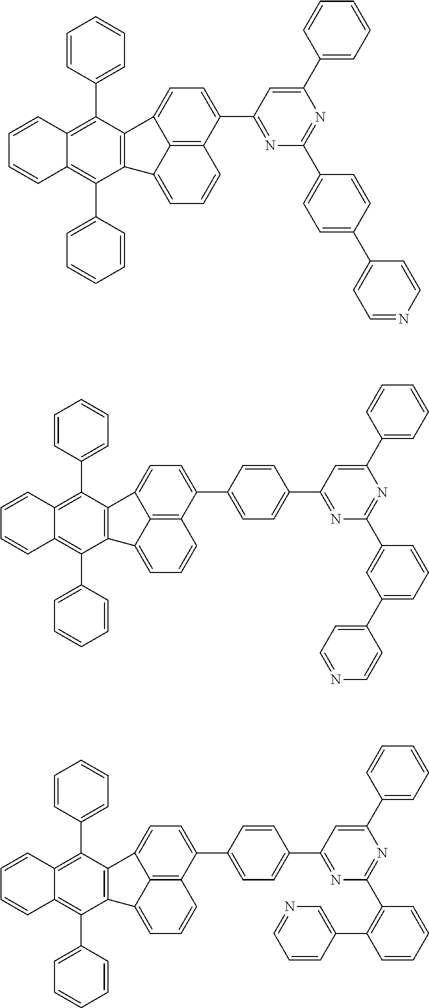 [SMILES: c1ccc(-c2cc(-c3ccc4c5c(cccc35)-c3c-4c(-c4ccccc4)c4ccccc4c3-c3ccccc3)nc(-c3ccc(-c4ccncc4)cc3)n2)cc1 ; c1ccc(-c2cc(-c3ccc(-c4ccc5c6c(cccc46)-c4c-5c(-c5ccccc5)c5ccccc5c4-c4ccccc4)cc3)nc(-c3cccc(-c4ccncc4)c3)n2)cc1 ; c1ccc(-c2cc(-c3ccc(-c4ccc5c6c(cccc46)-c4c-5c(-c5ccccc5)c5ccccc5c4-c4ccccc4)cc3)nc(-c3ccccc3-c3cccnc3)n2)cc1]

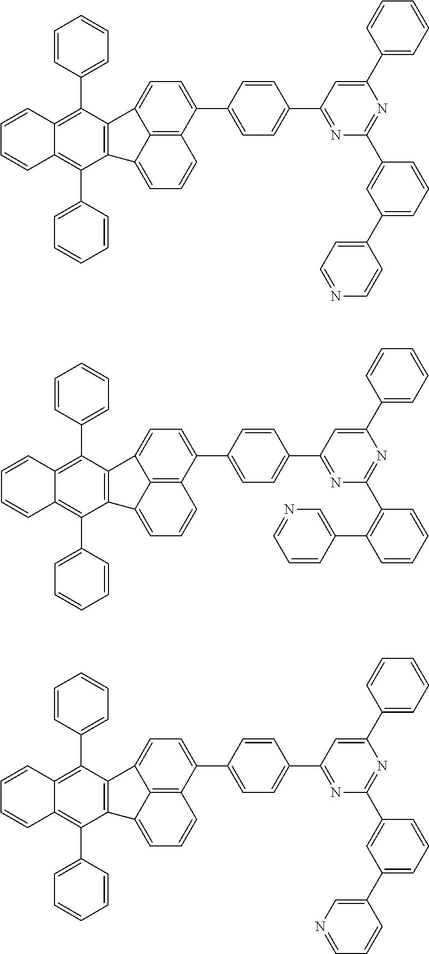 [SMILES: c1ccc(-c2cc(-c3ccc(-c4ccc5c6c(cccc46)-c4c-5c(-c5ccccc5)c5ccccc5c4-c4ccccc4)cc3)nc(-c3cccc(-c4ccncc4)c3)n2)cc1 ; c1ccc(-c2cc(-c3ccc(-c4ccc5c6c(cccc46)-c4c-5c(-c5ccccc5)c5ccccc5c4-c4ccccc4)cc3)nc(-c3ccccc3-c3cccnc3)n2)cc1 ; c1ccc(-c2cc(-c3ccc(-c4ccc5c6c(cccc46)-c4c-5c(-c5ccccc5)c5ccccc5c4-c4ccccc4)cc3)nc(-c3cccc(-c4cccnc4)c3)n2)cc1]

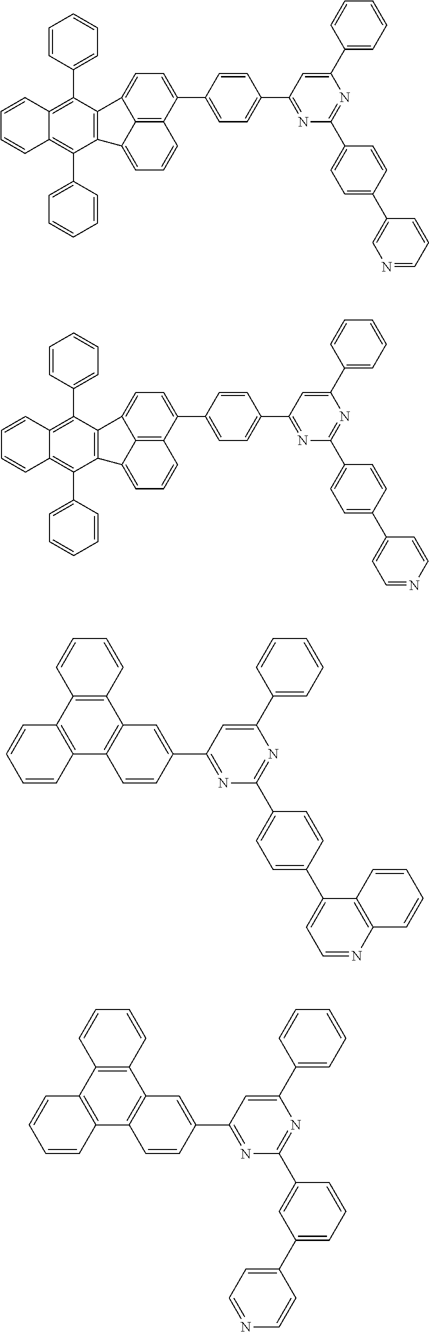 [SMILES: c1ccc(-c2cc(-c3ccc(-c4ccc5c6c(cccc46)-c4c-5c(-c5ccccc5)c5ccccc5c4-c4ccccc4)cc3)nc(-c3ccc(-c4cccnc4)cc3)n2)cc1 ; c1ccc(-c2cc(-c3ccc(-c4ccc5c6c(cccc46)-c4c-5c(-c5ccccc5)c5ccccc5c4-c4ccccc4)cc3)nc(-c3ccc(-c4ccncc4)cc3)n2)cc1 ; c1ccc(-c2cc(-c3ccc4c5ccccc5c5ccccc5c4c3)nc(-c3ccc(-c4ccnc5ccccc45)cc3)n2)cc1 ; c1ccc(-c2cc(-c3ccc4c5ccccc5c5ccccc5c4c3)nc(-c3cccc(-c4ccncc4)c3)n2)cc1]

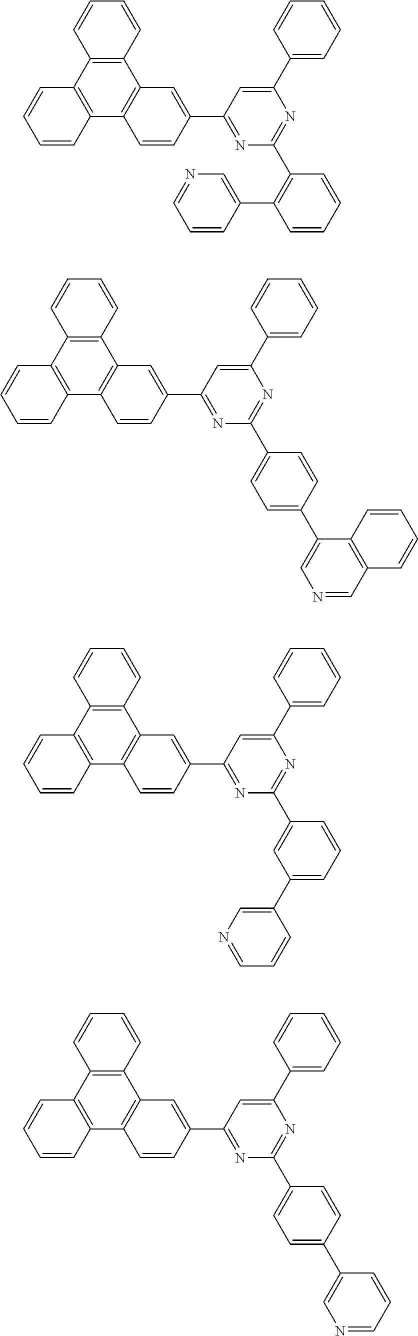 [SMILES: c1ccc(-c2cc(-c3ccc4c5ccccc5c5ccccc5c4c3)nc(-c3ccccc3-c3cccnc3)n2)cc1 ; c1ccc(-c2cc(-c3ccc4c5ccccc5c5ccccc5c4c3)nc(-c3ccc(-c4cncc5ccccc45)cc3)n2)cc1 ; c1ccc(-c2cc(-c3ccc4c5ccccc5c5ccccc5c4c3)nc(-c3cccc(-c4cccnc4)c3)n2)cc1 ; c1ccc(-c2cc(-c3ccc4c5ccccc5c5ccccc5c4c3)nc(-c3ccc(-c4cccnc4)cc3)n2)cc1]

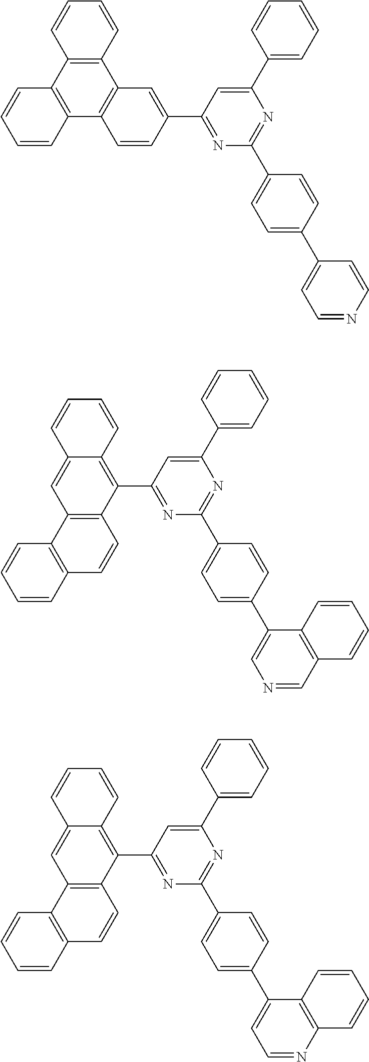 [SMILES: c1ccc(-c2cc(-c3ccc4c5ccccc5c5ccccc5c4c3)nc(-c3ccc(-c4ccncc4)cc3)n2)cc1 ; c1ccc(-c2cc(-c3c4ccccc4cc4c3ccc3ccccc34)nc(-c3ccc(-c4cncc5ccccc45)cc3)n2)cc1 ; c1ccc(-c2cc(-c3c4ccccc4cc4c3ccc3ccccc34)nc(-c3ccc(-c4ccnc5ccccc45)cc3)n2)cc1]

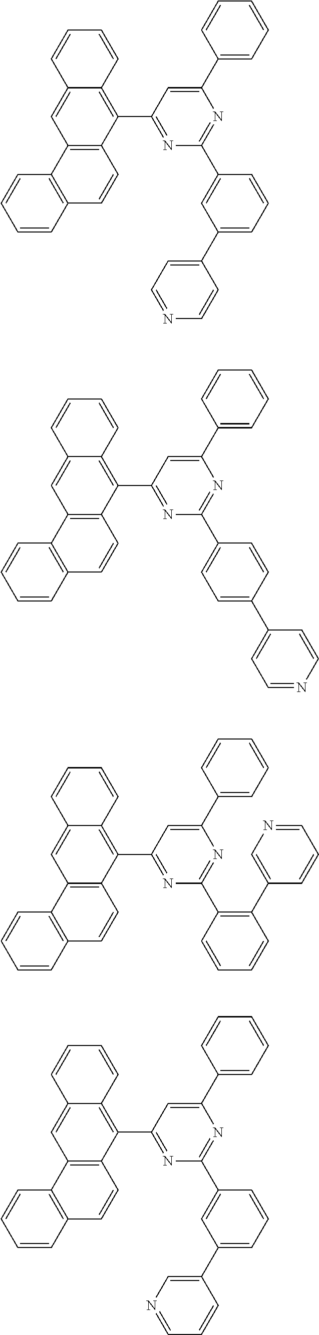 [SMILES: c1ccc(-c2cc(-c3c4ccccc4cc4c3ccc3ccccc34)nc(-c3cccc(-c4ccncc4)c3)n2)cc1 ; c1ccc(-c2cc(-c3c4ccccc4cc4c3ccc3ccccc34)nc(-c3ccc(-c4ccncc4)cc3)n2)cc1 ; c1ccc(-c2cc(-c3c4ccccc4cc4c3ccc3ccccc34)nc(-c3ccccc3-c3cccnc3)n2)cc1 ; c1ccc(-c2cc(-c3c4ccccc4cc4c3ccc3ccccc34)nc(-c3cccc(-c4cccnc4)c3)n2)cc1]

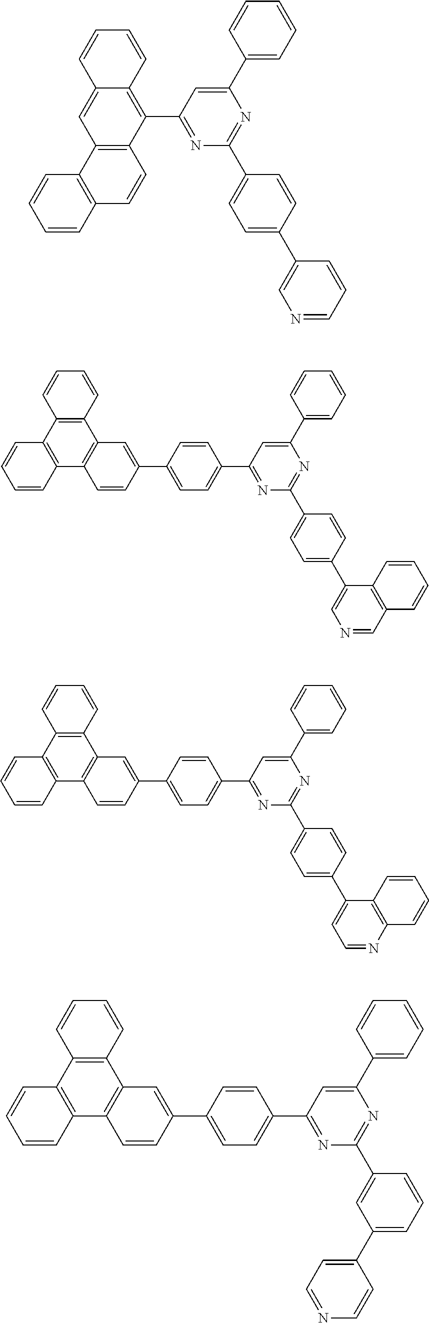 [SMILES: c1ccc(-c2cc(-c3c4ccccc4cc4c3ccc3ccccc34)nc(-c3ccc(-c4cccnc4)cc3)n2)cc1 ; c1ccc(-c2cc(-c3ccc(-c4ccc5c6ccccc6c6ccccc6c5c4)cc3)nc(-c3ccc(-c4cncc5ccccc45)cc3)n2)cc1 ; c1ccc(-c2cc(-c3ccc(-c4ccc5c6ccccc6c6ccccc6c5c4)cc3)nc(-c3ccc(-c4ccnc5ccccc45)cc3)n2)cc1 ; c1ccc(-c2cc(-c3ccc(-c4ccc5c6ccccc6c6ccccc6c5c4)cc3)nc(-c3cccc(-c4ccncc4)c3)n2)cc1]

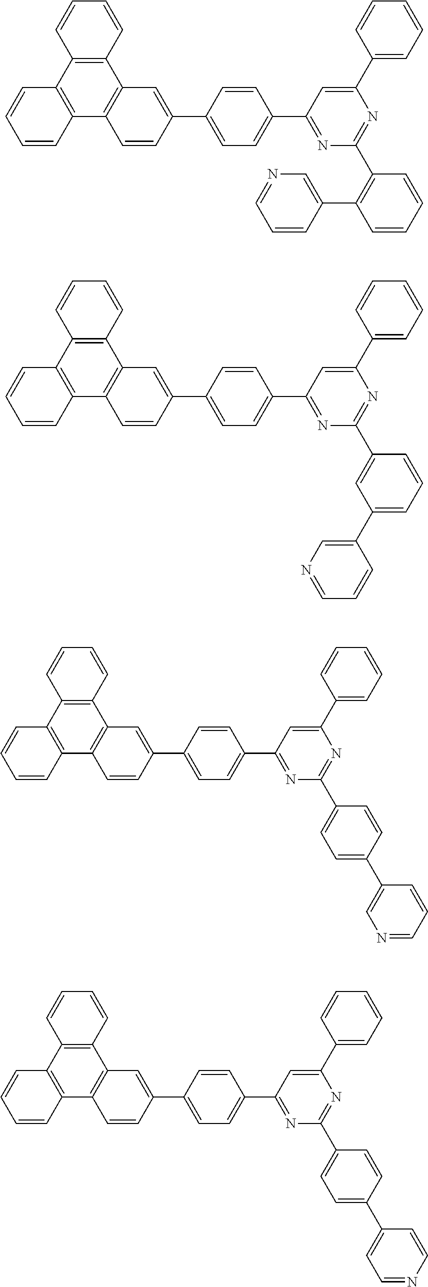 [SMILES: c1ccc(-c2cc(-c3ccc(-c4ccc5c6ccccc6c6ccccc6c5c4)cc3)nc(-c3ccccc3-c3cccnc3)n2)cc1 ; c1ccc(-c2cc(-c3ccc(-c4ccc5c6ccccc6c6ccccc6c5c4)cc3)nc(-c3cccc(-c4cccnc4)c3)n2)cc1 ; c1ccc(-c2cc(-c3ccc(-c4ccc5c6ccccc6c6ccccc6c5c4)cc3)nc(-c3ccc(-c4cccnc4)cc3)n2)cc1 ; c1ccc(-c2cc(-c3ccc(-c4ccc5c6ccccc6c6ccccc6c5c4)cc3)nc(-c3ccc(-c4ccncc4)cc3)n2)cc1]

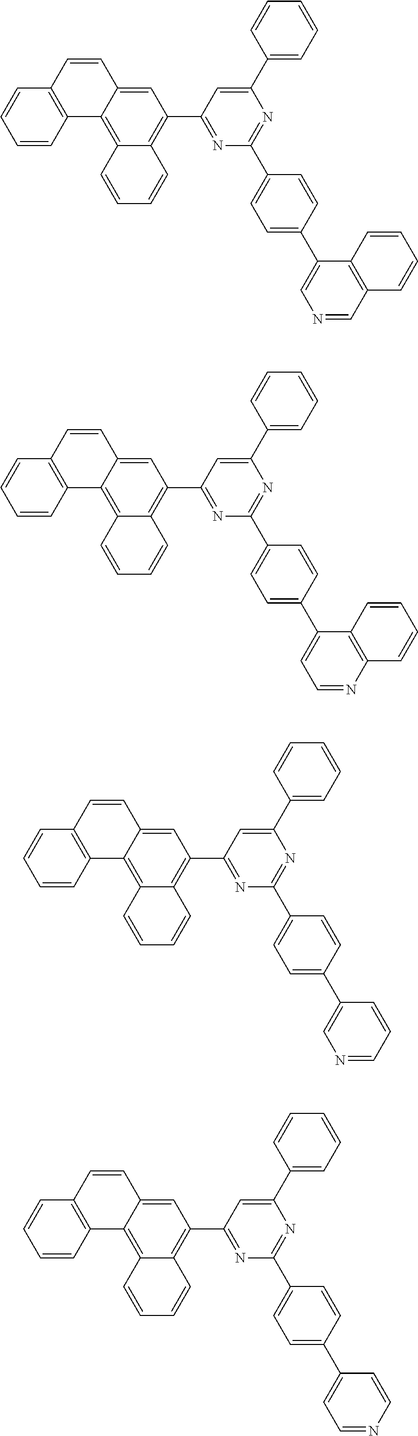 [SMILES: c1ccc(-c2cc(-c3cc4ccc5ccccc5c4c4ccccc34)nc(-c3ccc(-c4cncc5ccccc45)cc3)n2)cc1 ; c1ccc(-c2cc(-c3cc4ccc5ccccc5c4c4ccccc34)nc(-c3ccc(-c4ccnc5ccccc45)cc3)n2)cc1 ; c1ccc(-c2cc(-c3cc4ccc5ccccc5c4c4ccccc34)nc(-c3ccc(-c4cccnc4)cc3)n2)cc1 ; c1ccc(-c2cc(-c3cc4ccc5ccccc5c4c4ccccc34)nc(-c3ccc(-c4ccncc4)cc3)n2)cc1]

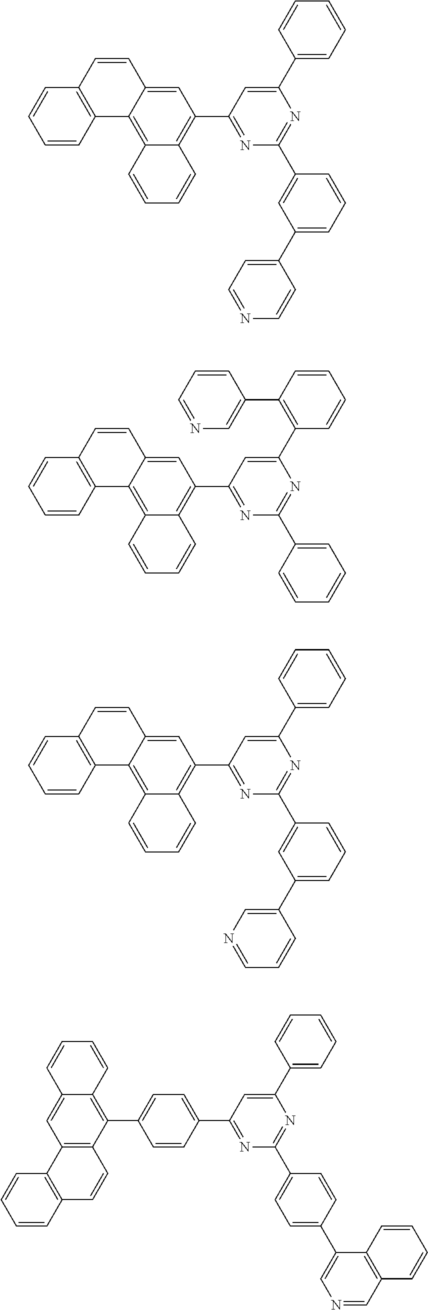 [SMILES: c1ccc(-c2cc(-c3cc4ccc5ccccc5c4c4ccccc34)nc(-c3cccc(-c4ccncc4)c3)n2)cc1 ; c1ccc(-c2nc(-c3ccccc3-c3cccnc3)cc(-c3cc4ccc5ccccc5c4c4ccccc34)n2)cc1 ; c1ccc(-c2cc(-c3cc4ccc5ccccc5c4c4ccccc34)nc(-c3cccc(-c4cccnc4)c3)n2)cc1 ; c1ccc(-c2cc(-c3ccc(-c4c5ccccc5cc5c4ccc4ccccc45)cc3)nc(-c3ccc(-c4cncc5ccccc45)cc3)n2)cc1]

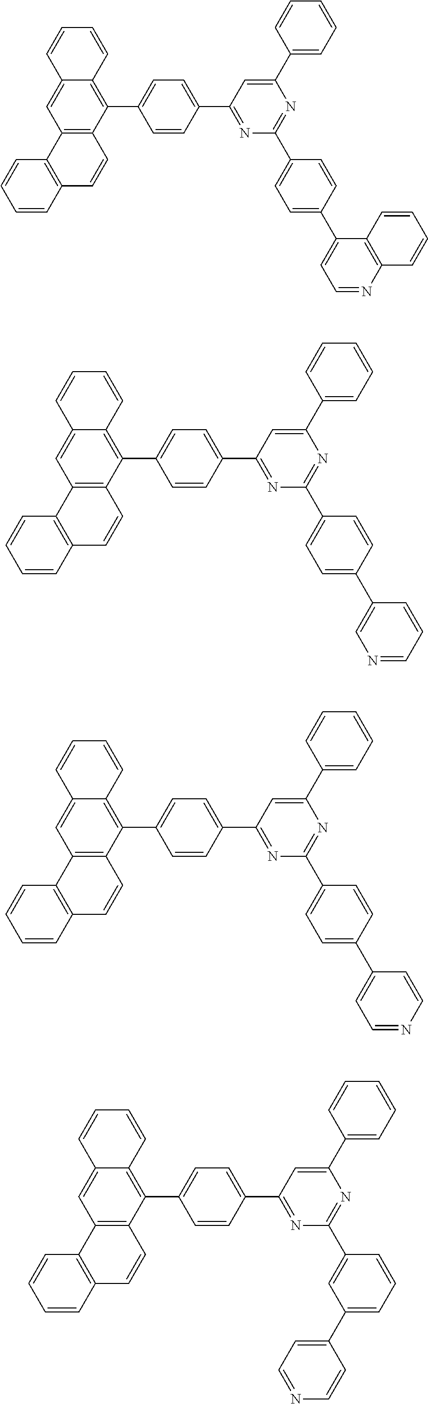 [SMILES: c1ccc(-c2cc(-c3ccc(-c4c5ccccc5cc5c4ccc4ccccc45)cc3)nc(-c3ccc(-c4ccnc5ccccc45)cc3)n2)cc1 ; c1ccc(-c2cc(-c3ccc(-c4c5ccccc5cc5c4ccc4ccccc45)cc3)nc(-c3ccc(-c4cccnc4)cc3)n2)cc1 ; c1ccc(-c2cc(-c3ccc(-c4c5ccccc5cc5c4ccc4ccccc45)cc3)nc(-c3ccc(-c4ccncc4)cc3)n2)cc1 ; c1ccc(-c2cc(-c3ccc(-c4c5ccccc5cc5c4ccc4ccccc45)cc3)nc(-c3cccc(-c4ccncc4)c3)n2)cc1]

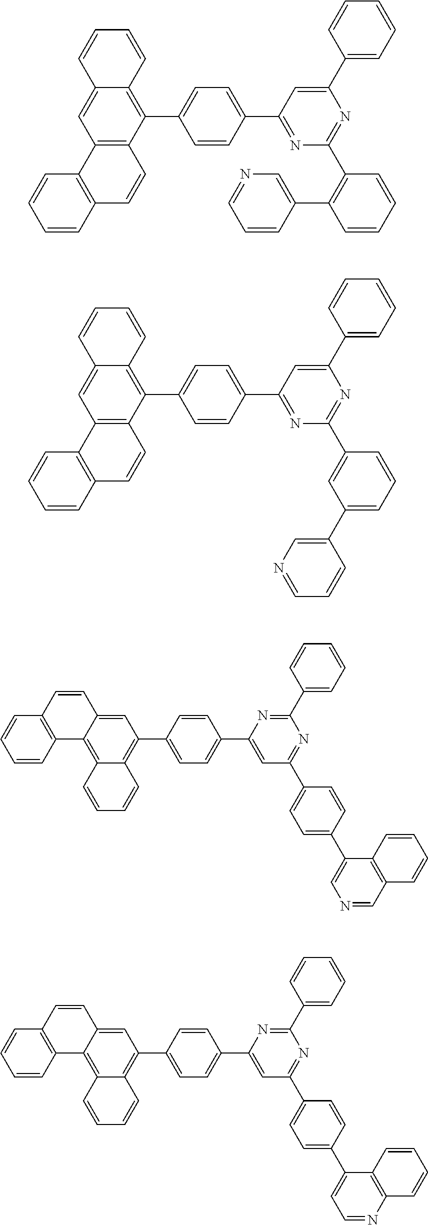 [SMILES: c1ccc(-c2cc(-c3ccc(-c4c5ccccc5cc5c4ccc4ccccc45)cc3)nc(-c3ccccc3-c3cccnc3)n2)cc1 ; c1ccc(-c2cc(-c3ccc(-c4c5ccccc5cc5c4ccc4ccccc45)cc3)nc(-c3cccc(-c4cccnc4)c3)n2)cc1 ; c1ccc(-c2nc(-c3ccc(-c4cncc5ccccc45)cc3)cc(-c3ccc(-c4cc5ccc6ccccc6c5c5ccccc45)cc3)n2)cc1 ; c1ccc(-c2nc(-c3ccc(-c4ccnc5ccccc45)cc3)cc(-c3ccc(-c4cc5ccc6ccccc6c5c5ccccc45)cc3)n2)cc1]

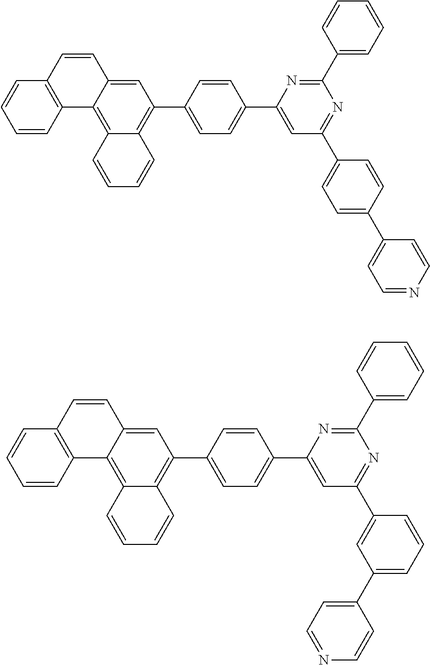 [SMILES: c1ccc(-c2nc(-c3ccc(-c4ccncc4)cc3)cc(-c3ccc(-c4cc5ccc6ccccc6c5c5ccccc45)cc3)n2)cc1 ; c1ccc(-c2nc(-c3ccc(-c4cc5ccc6ccccc6c5c5ccccc45)cc3)cc(-c3cccc(-c4ccncc4)c3)n2)cc1]

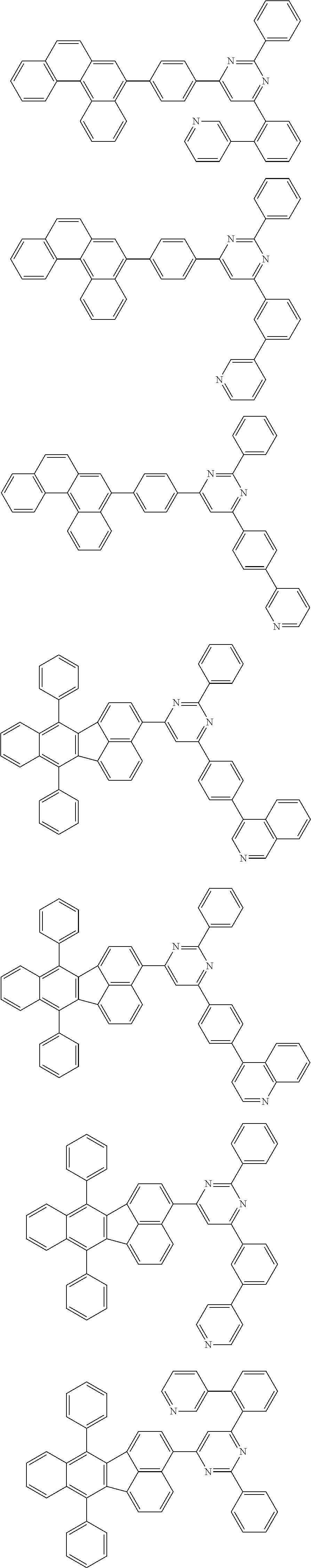

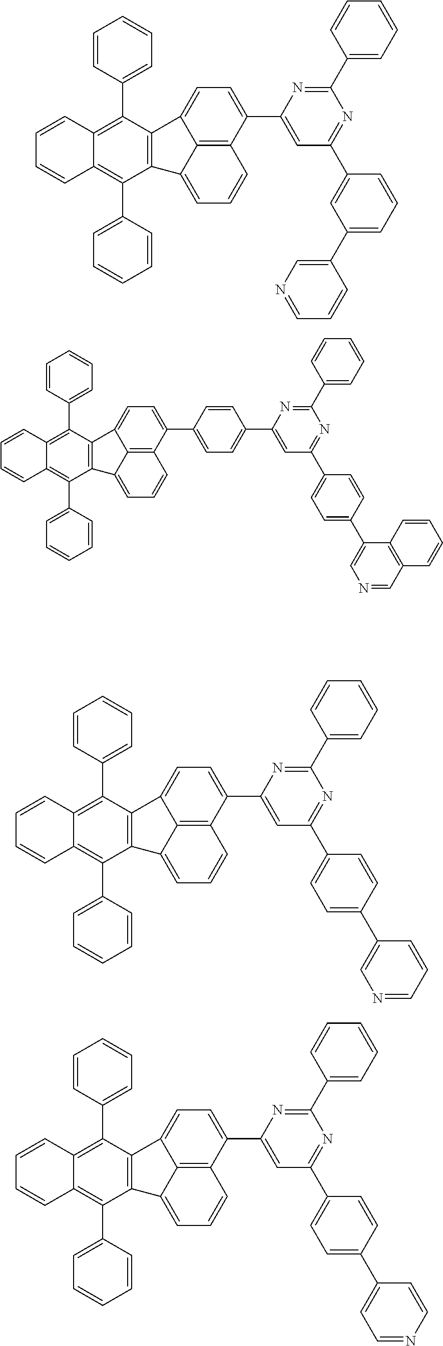

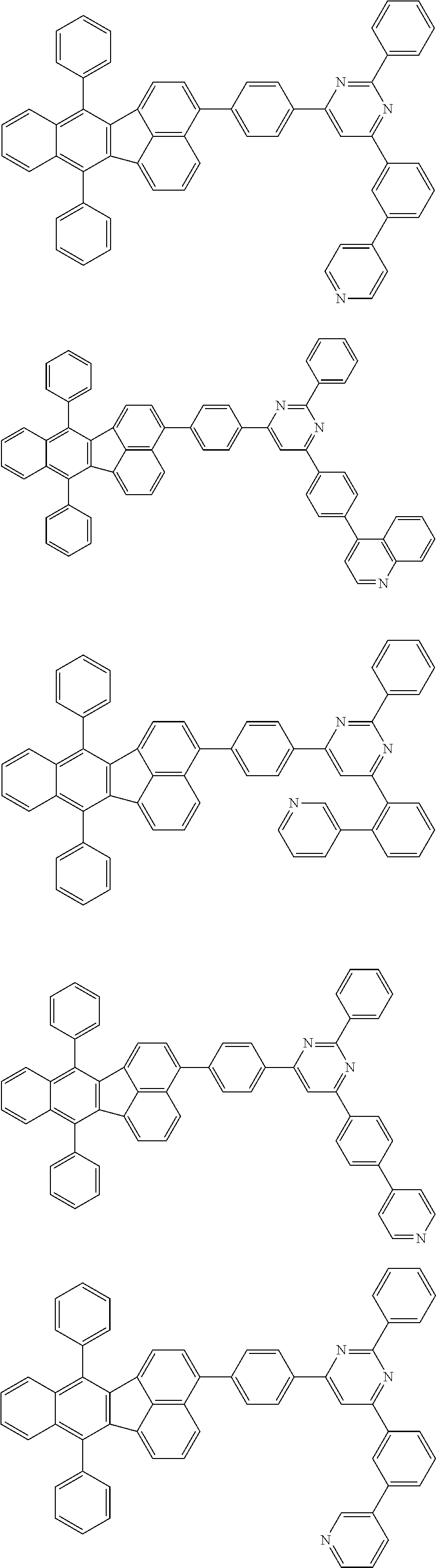

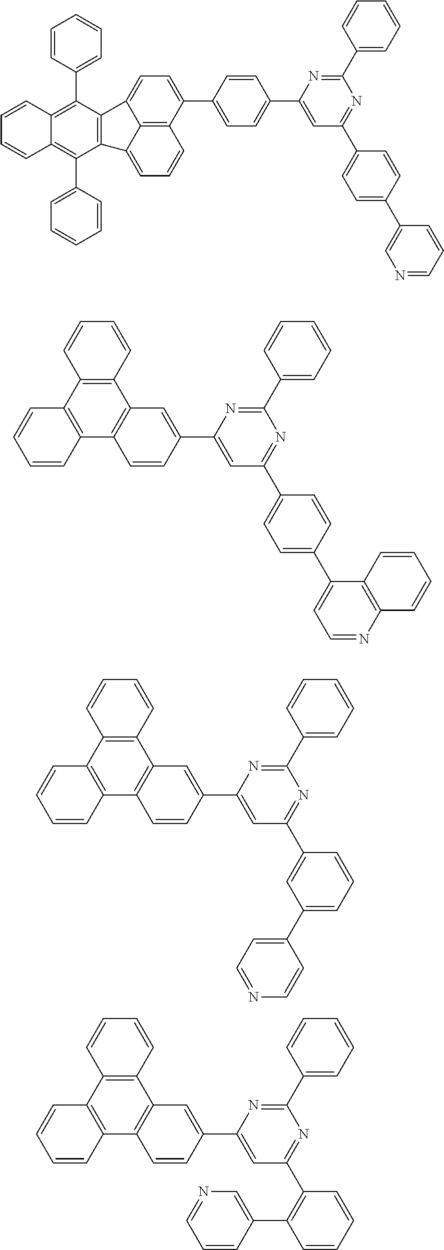

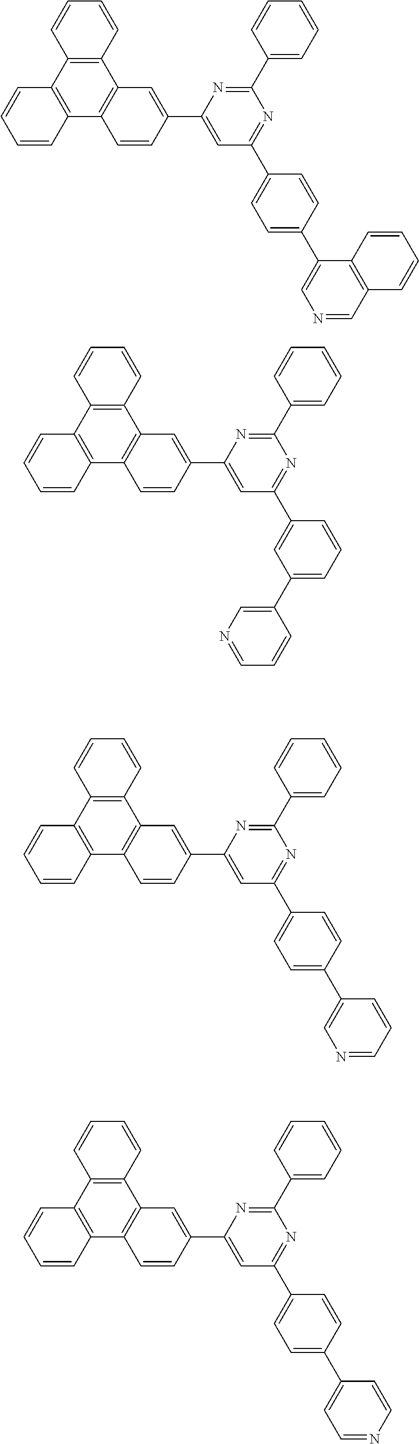

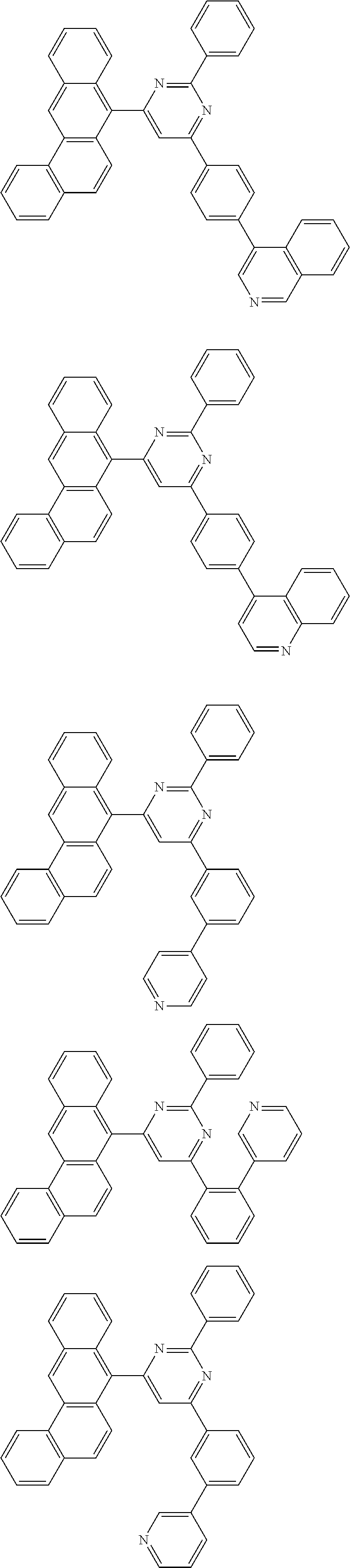

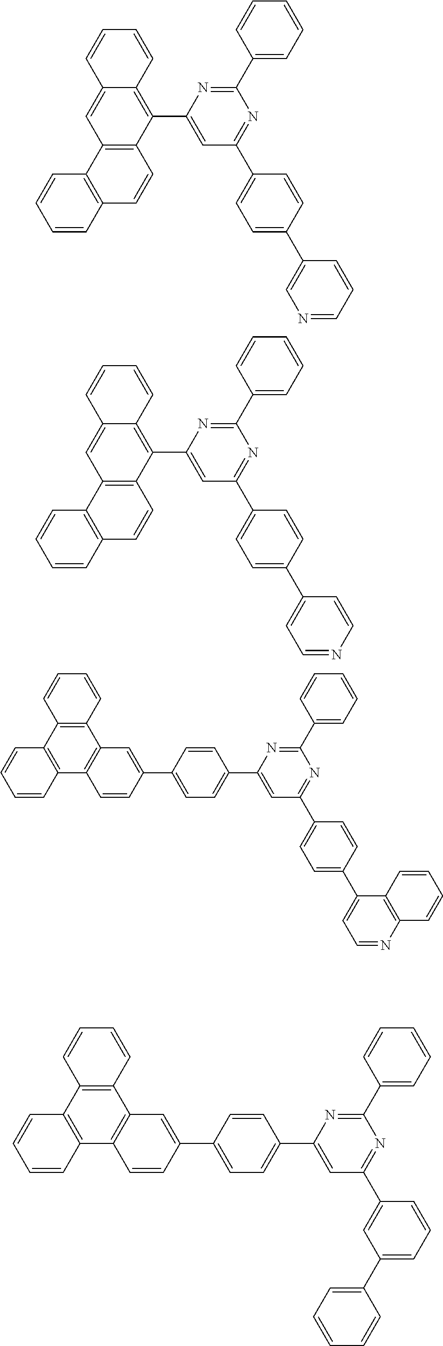

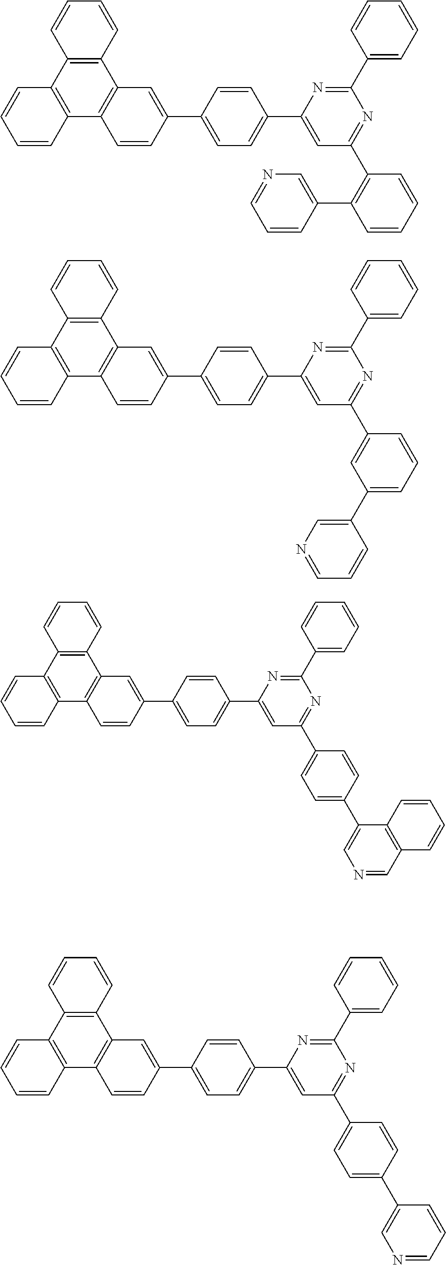

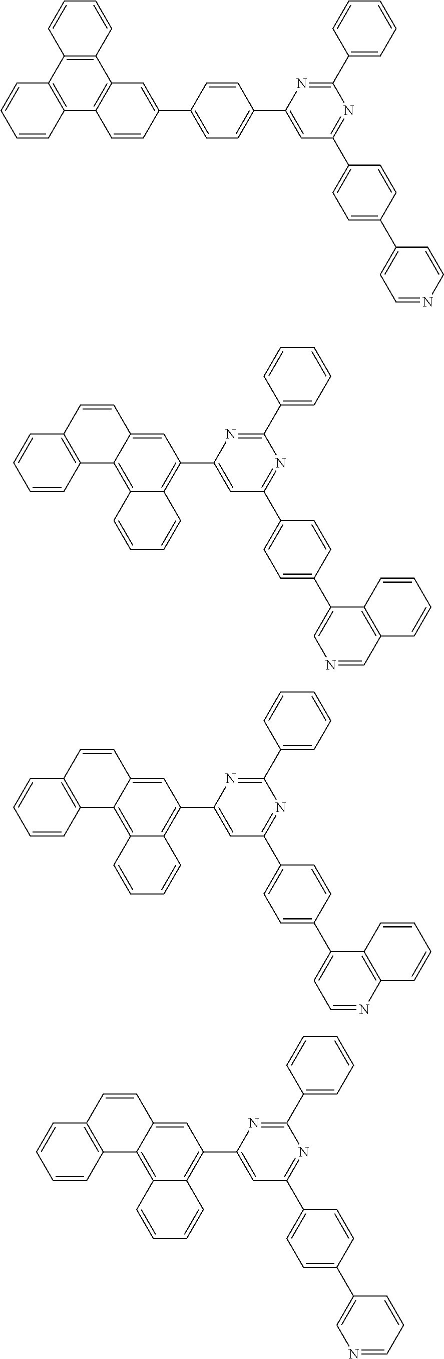

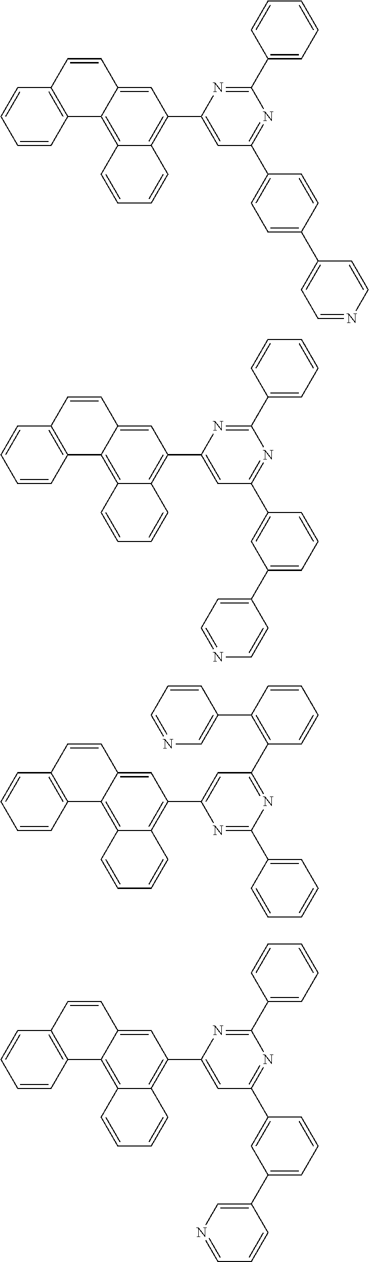

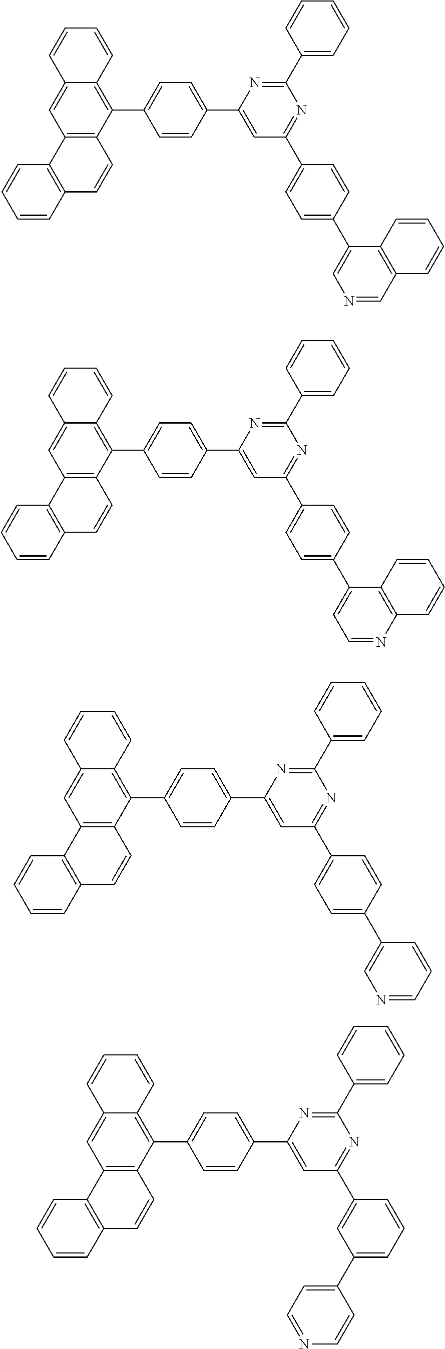

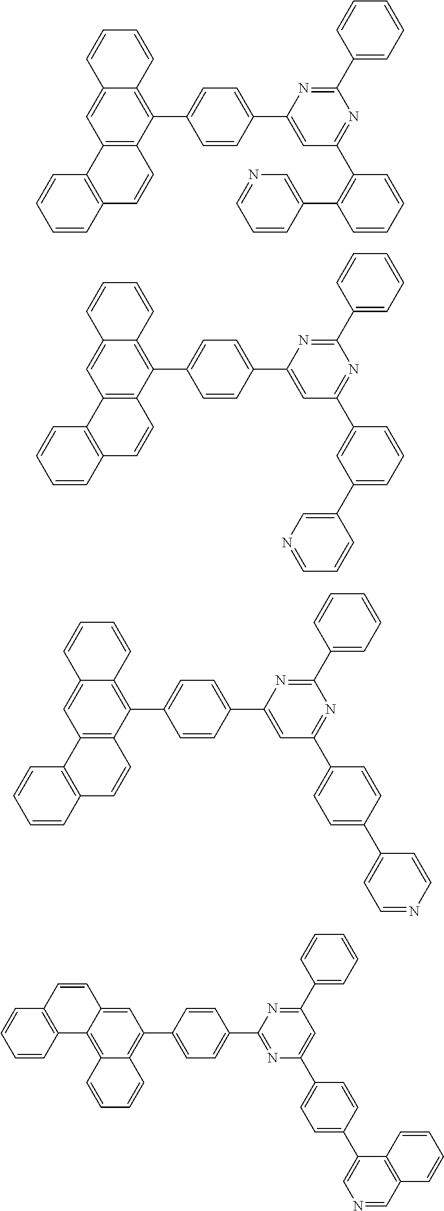

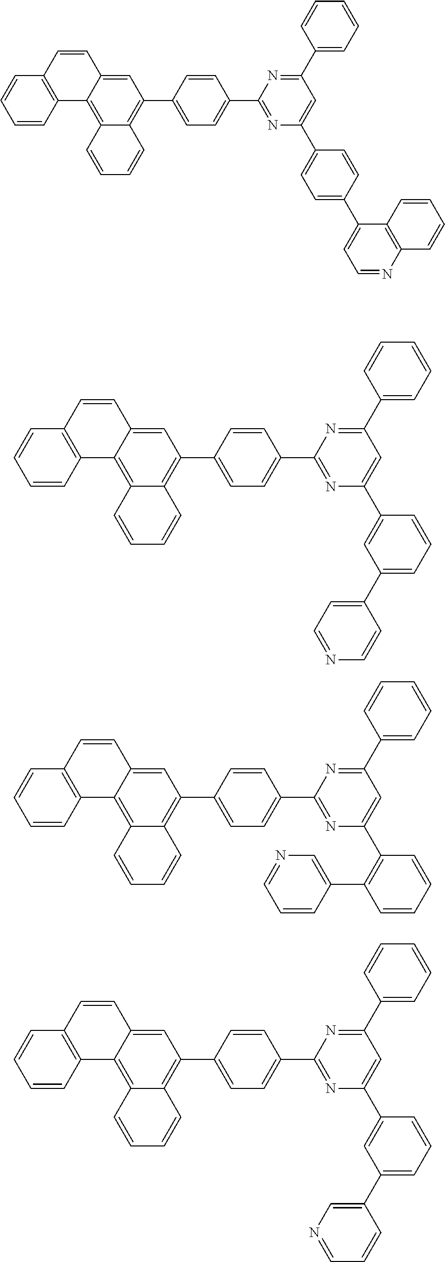

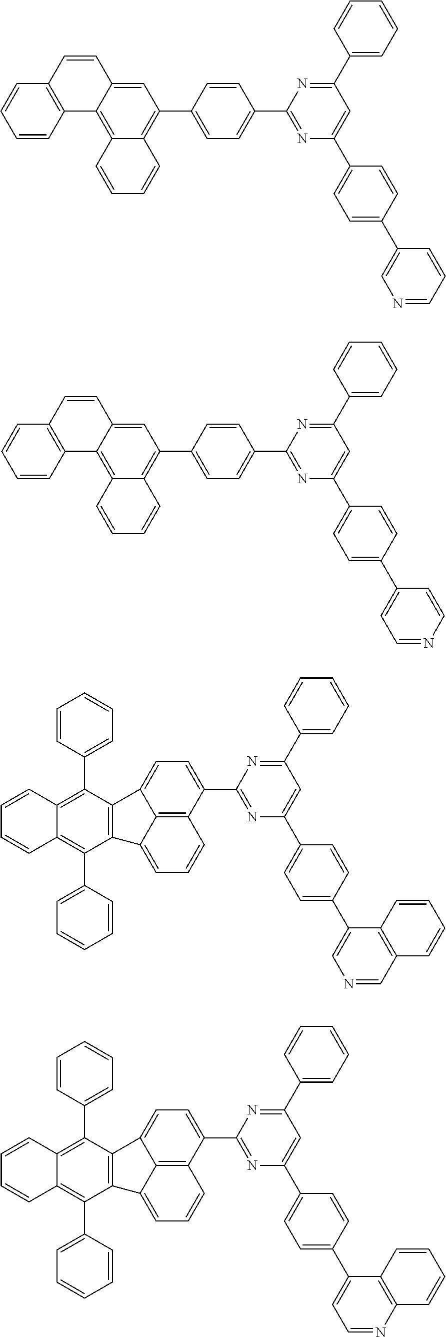

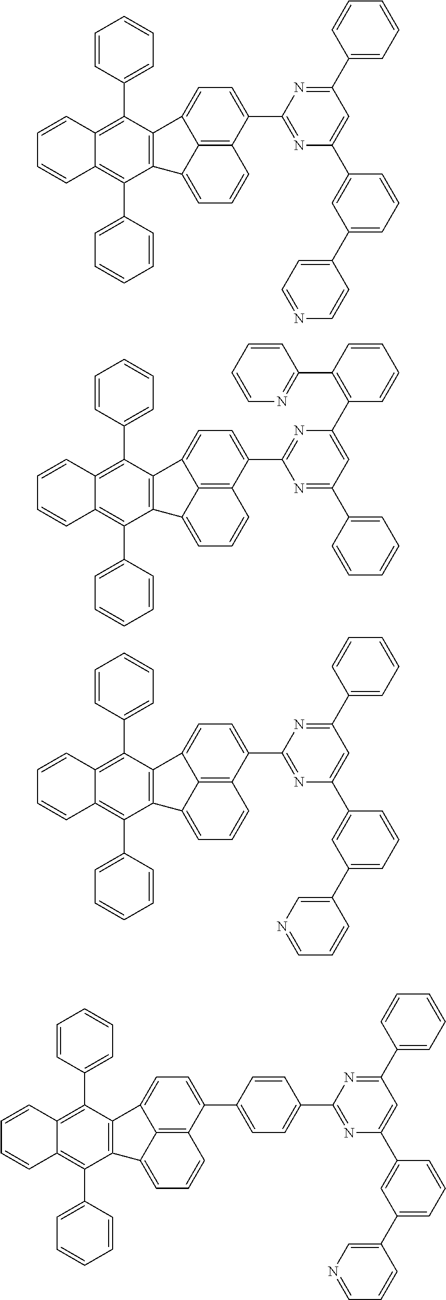

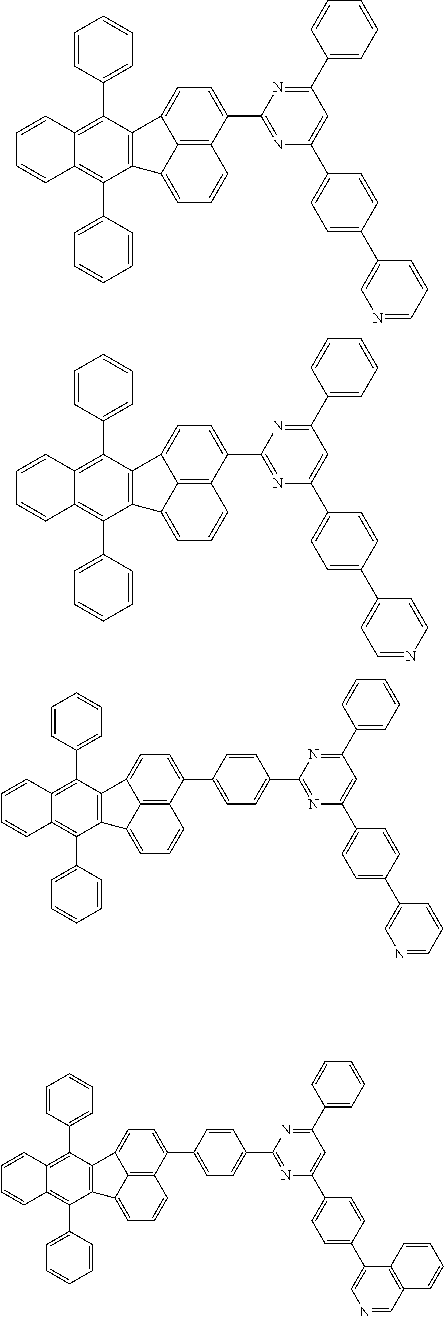

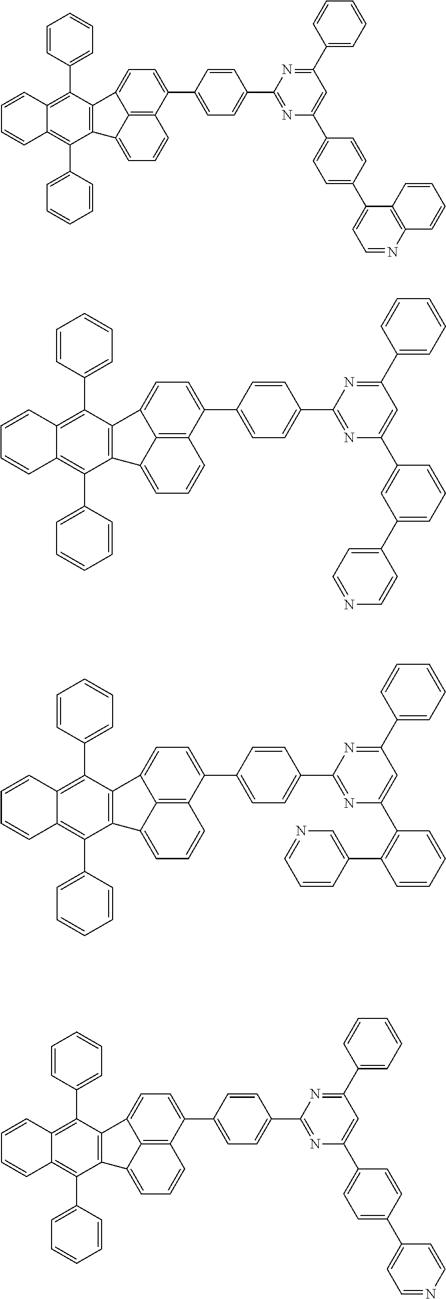 [SMILES: c1ccc(-c2cc(-c3ccc(-c4ccnc5ccccc45)cc3)nc(-c3ccc(-c4ccc5c6c(cccc46)-c4c-5c(-c5ccccc5)c5ccccc5c4-c4ccccc4)cc3)n2)cc1 ; c1ccc(-c2cc(-c3cccc(-c4ccncc4)c3)nc(-c3ccc(-c4ccc5c6c(cccc46)-c4c-5c(-c5ccccc5)c5ccccc5c4-c4ccccc4)cc3)n2)cc1 ; c1ccc(-c2cc(-c3ccccc3-c3cccnc3)nc(-c3ccc(-c4ccc5c6c(cccc46)-c4c-5c(-c5ccccc5)c5ccccc5c4-c4ccccc4)cc3)n2)cc1 ; c1ccc(-c2cc(-c3ccc(-c4ccncc4)cc3)nc(-c3ccc(-c4ccc5c6c(cccc46)-c4c-5c(-c5ccccc5)c5ccccc5c4-c4ccccc4)cc3)n2)cc1]

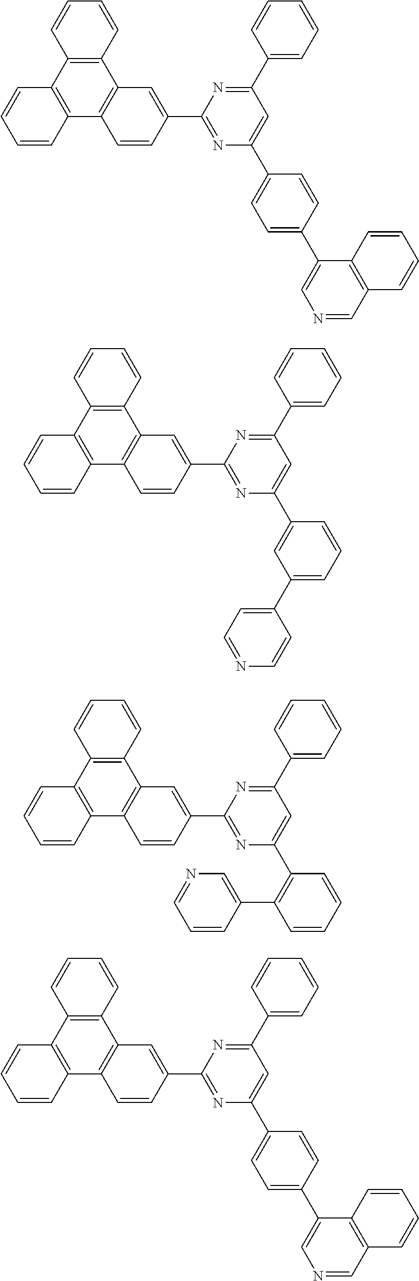

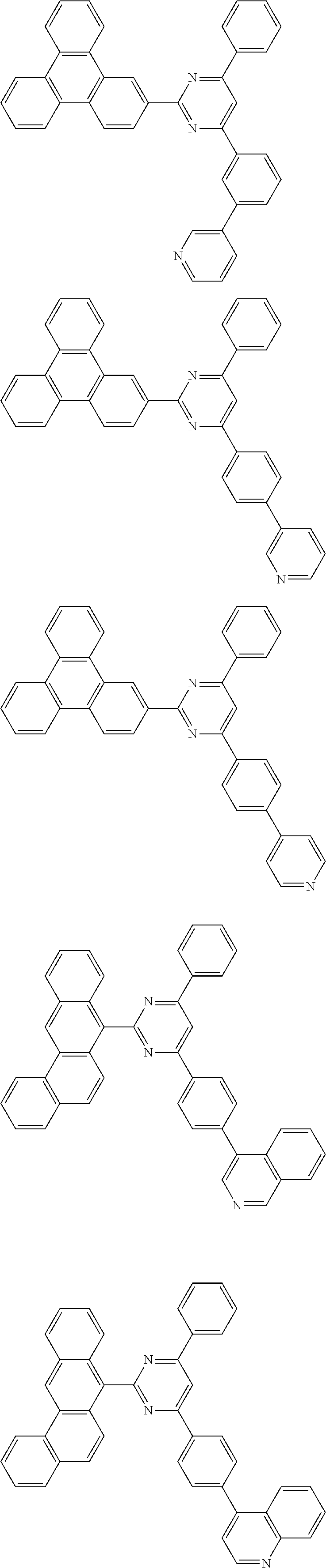

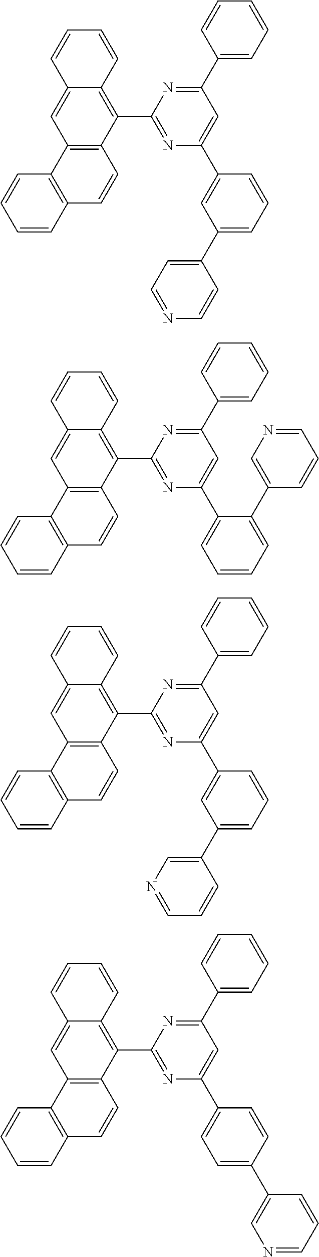

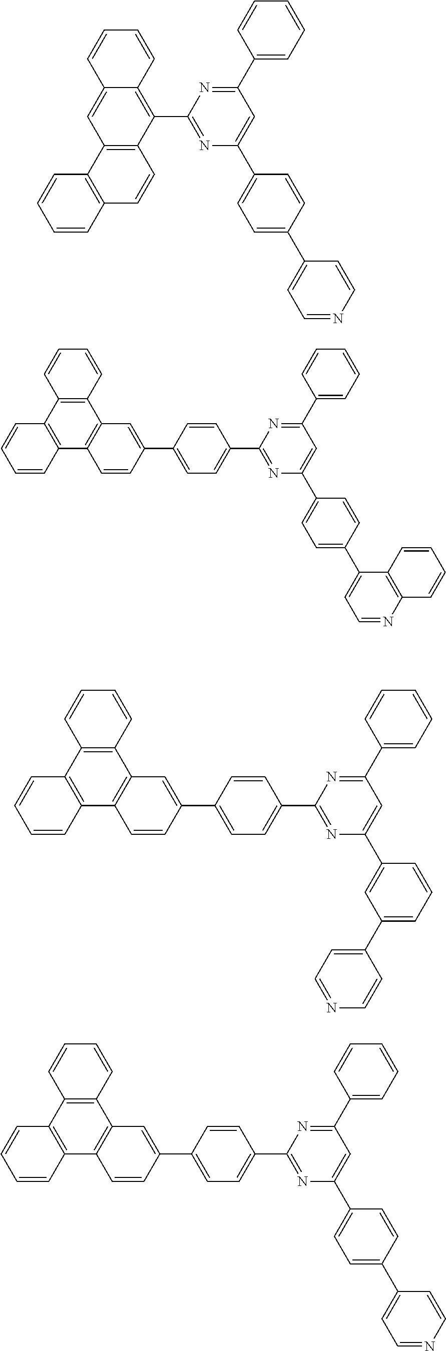

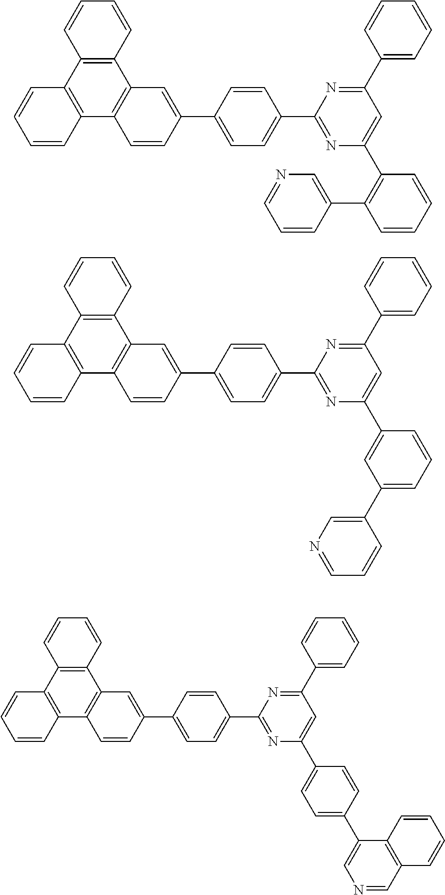

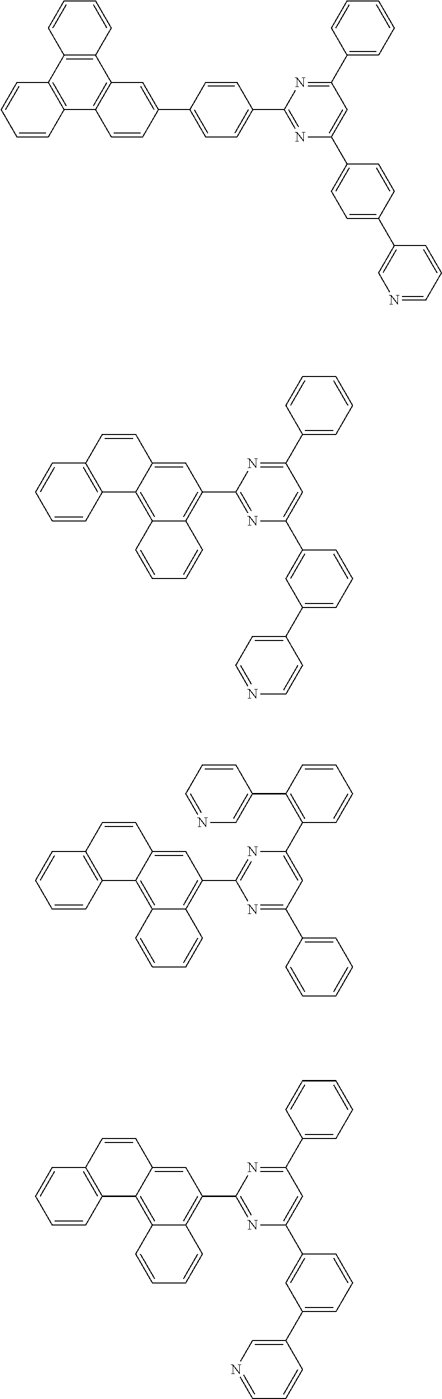 [SMILES: c1ccc(-c2cc(-c3ccc(-c4cccnc4)cc3)nc(-c3ccc(-c4ccc5c6ccccc6c6ccccc6c5c4)cc3)n2)cc1 ; c1ccc(-c2cc(-c3cccc(-c4ccncc4)c3)nc(-c3cc4ccc5ccccc5c4c4ccccc34)n2)cc1 ; c1ccc(-c2cc(-c3ccccc3-c3cccnc3)nc(-c3cc4ccc5ccccc5c4c4ccccc34)n2)cc1 ; c1ccc(-c2cc(-c3cccc(-c4cccnc4)c3)nc(-c3cc4ccc5ccccc5c4c4ccccc34)n2)cc1]

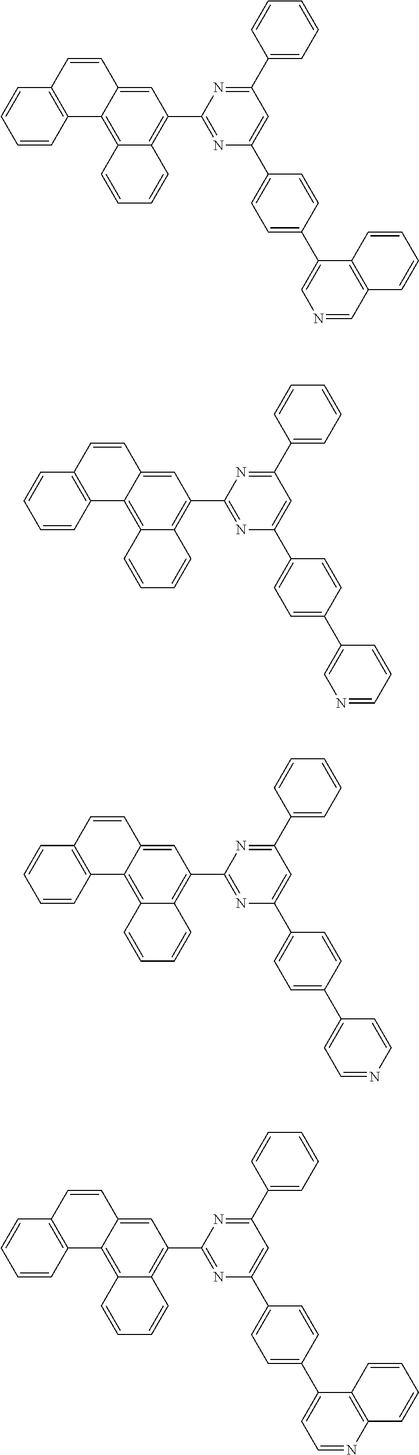 [SMILES: c1ccc(-c2cc(-c3ccc(-c4cncc5ccccc45)cc3)nc(-c3cc4ccc5ccccc5c4c4ccccc34)n2)cc1 ; c1ccc(-c2cc(-c3ccc(-c4cccnc4)cc3)nc(-c3cc4ccc5ccccc5c4c4ccccc34)n2)cc1 ; c1ccc(-c2cc(-c3ccc(-c4ccncc4)cc3)nc(-c3cc4ccc5ccccc5c4c4ccccc34)n2)cc1 ; c1ccc(-c2cc(-c3ccc(-c4ccnc5ccccc45)cc3)nc(-c3cc4ccc5ccccc5c4c4ccccc34)n2)cc1]

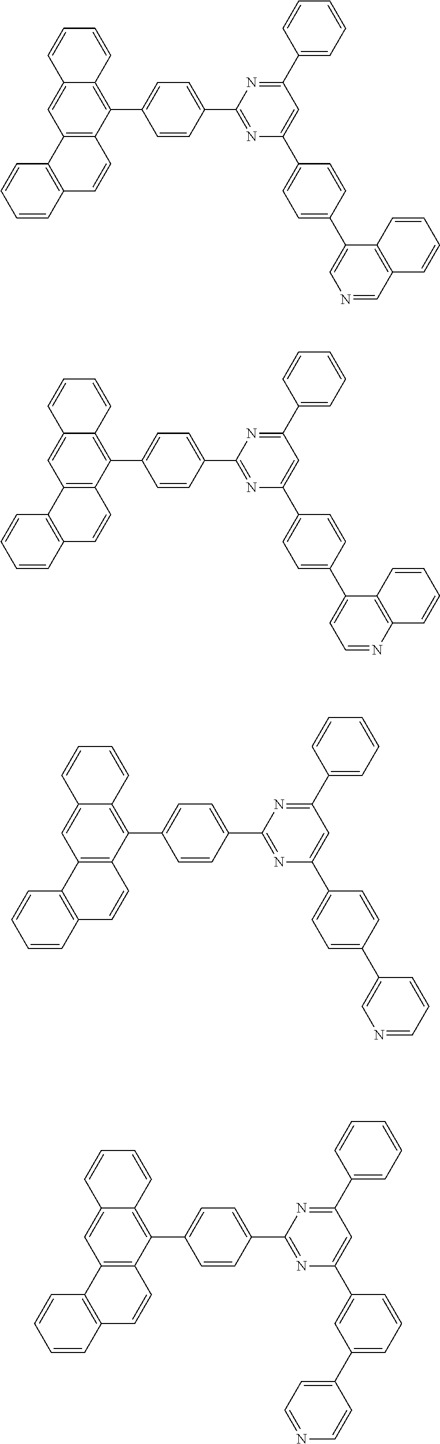 [SMILES: c1ccc(-c2cc(-c3ccc(-c4cncc5ccccc45)cc3)nc(-c3ccc(-c4c5ccccc5cc5c4ccc4ccccc45)cc3)n2)cc1 ; c1ccc(-c2cc(-c3ccc(-c4ccnc5ccccc45)cc3)nc(-c3ccc(-c4c5ccccc5cc5c4ccc4ccccc45)cc3)n2)cc1 ; c1ccc(-c2cc(-c3ccc(-c4cccnc4)cc3)nc(-c3ccc(-c4c5ccccc5cc5c4ccc4ccccc45)cc3)n2)cc1 ; c1ccc(-c2cc(-c3cccc(-c4ccncc4)c3)nc(-c3ccc(-c4c5ccccc5cc5c4ccc4ccccc45)cc3)n2)cc1]

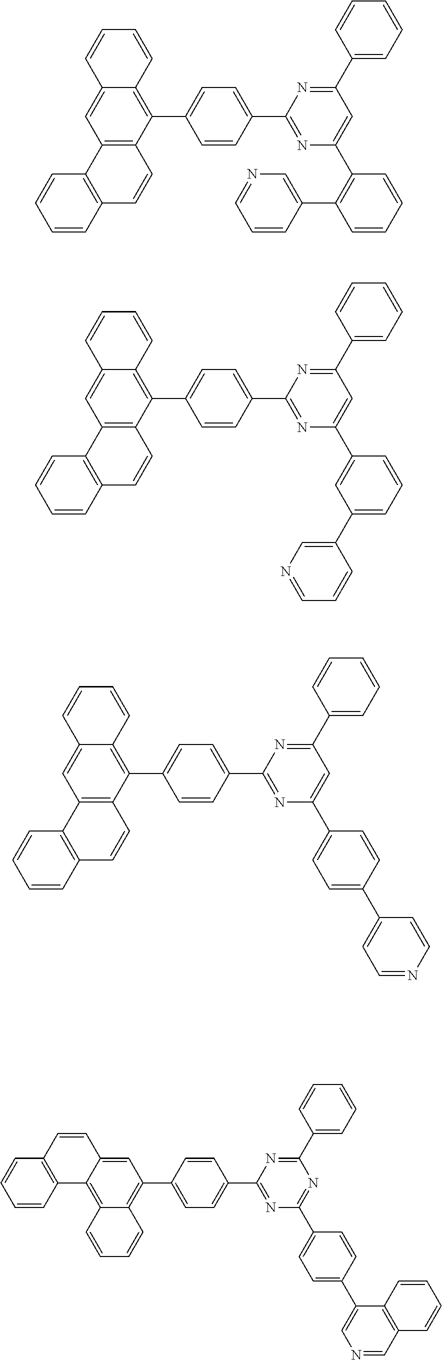 [SMILES: c1ccc(-c2cc(-c3ccccc3-c3cccnc3)nc(-c3ccc(-c4c5ccccc5cc5c4ccc4ccccc45)cc3)n2)cc1 ; c1ccc(-c2cc(-c3cccc(-c4cccnc4)c3)nc(-c3ccc(-c4c5ccccc5cc5c4ccc4ccccc45)cc3)n2)cc1 ; c1ccc(-c2cc(-c3ccc(-c4ccncc4)cc3)nc(-c3ccc(-c4c5ccccc5cc5c4ccc4ccccc45)cc3)n2)cc1 ; c1ccc(-c2nc(-c3ccc(-c4cncc5ccccc45)cc3)nc(-c3ccc(-c4cc5ccc6ccccc6c5c5ccccc45)cc3)n2)cc1]

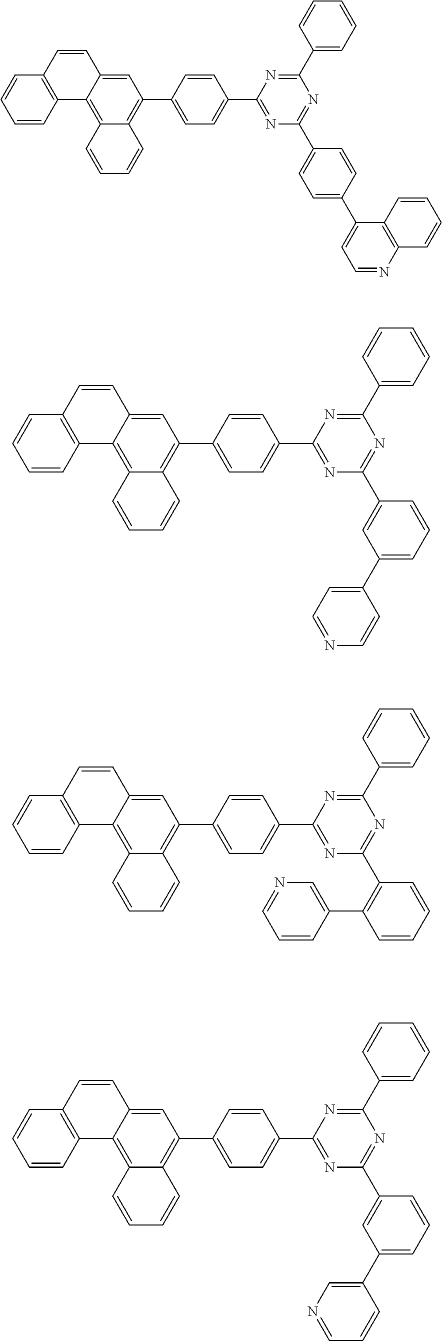 [SMILES: c1ccc(-c2nc(-c3ccc(-c4ccnc5ccccc45)cc3)nc(-c3ccc(-c4cc5ccc6ccccc6c5c5ccccc45)cc3)n2)cc1 ; c1ccc(-c2nc(-c3ccc(-c4cc5ccc6ccccc6c5c5ccccc45)cc3)nc(-c3cccc(-c4ccncc4)c3)n2)cc1 ; c1ccc(-c2nc(-c3ccc(-c4cc5ccc6ccccc6c5c5ccccc45)cc3)nc(-c3ccccc3-c3cccnc3)n2)cc1 ; c1ccc(-c2nc(-c3ccc(-c4cc5ccc6ccccc6c5c5ccccc45)cc3)nc(-c3cccc(-c4cccnc4)c3)n2)cc1]

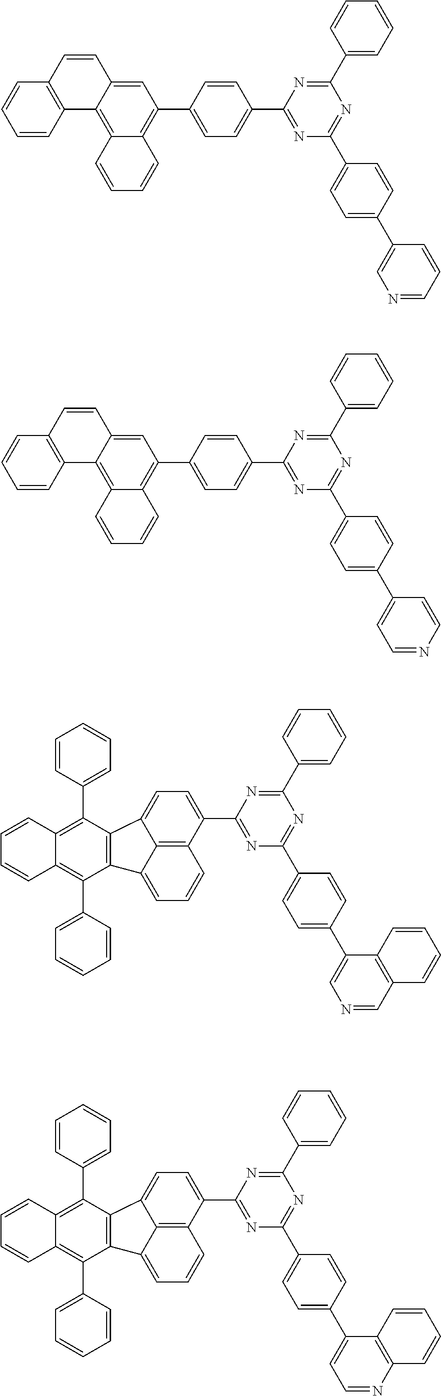 [SMILES: c1ccc(-c2nc(-c3ccc(-c4cccnc4)cc3)nc(-c3ccc(-c4cc5ccc6ccccc6c5c5ccccc45)cc3)n2)cc1 ; c1ccc(-c2nc(-c3ccc(-c4ccncc4)cc3)nc(-c3ccc(-c4cc5ccc6ccccc6c5c5ccccc45)cc3)n2)cc1 ; c1ccc(-c2nc(-c3ccc(-c4cncc5ccccc45)cc3)nc(-c3ccc4c5c(cccc35)-c3c-4c(-c4ccccc4)c4ccccc4c3-c3ccccc3)n2)cc1 ; c1ccc(-c2nc(-c3ccc(-c4ccnc5ccccc45)cc3)nc(-c3ccc4c5c(cccc35)-c3c-4c(-c4ccccc4)c4ccccc4c3-c3ccccc3)n2)cc1]

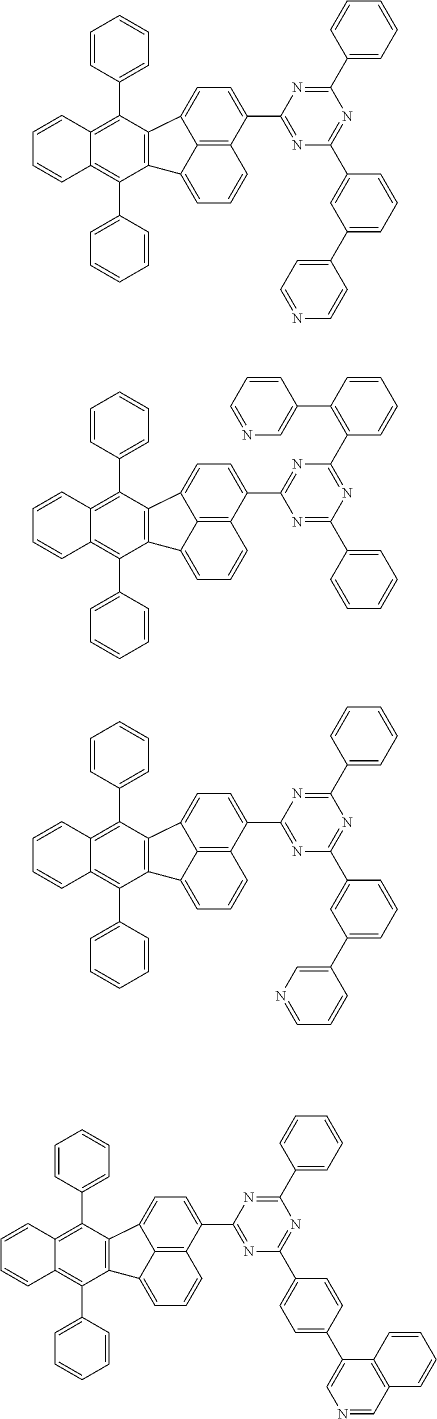 [SMILES: c1ccc(-c2nc(-c3cccc(-c4ccncc4)c3)nc(-c3ccc4c5c(cccc35)-c3c-4c(-c4ccccc4)c4ccccc4c3-c3ccccc3)n2)cc1 ; c1ccc(-c2nc(-c3ccccc3-c3cccnc3)nc(-c3ccc4c5c(cccc35)-c3c-4c(-c4ccccc4)c4ccccc4c3-c3ccccc3)n2)cc1 ; c1ccc(-c2nc(-c3cccc(-c4cccnc4)c3)nc(-c3ccc4c5c(cccc35)-c3c-4c(-c4ccccc4)c4ccccc4c3-c3ccccc3)n2)cc1 ; c1ccc(-c2nc(-c3ccc(-c4cncc5ccccc45)cc3)nc(-c3ccc4c5c(cccc35)-c3c-4c(-c4ccccc4)c4ccccc4c3-c3ccccc3)n2)cc1]

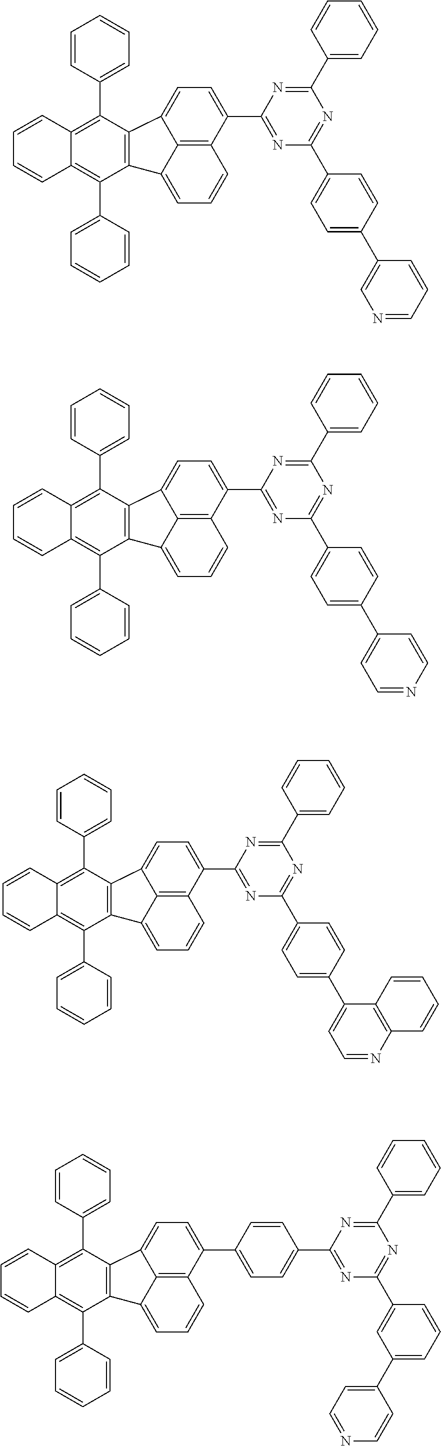 [SMILES: c1ccc(-c2nc(-c3ccc(-c4cccnc4)cc3)nc(-c3ccc4c5c(cccc35)-c3c-4c(-c4ccccc4)c4ccccc4c3-c3ccccc3)n2)cc1 ; c1ccc(-c2nc(-c3ccc(-c4ccncc4)cc3)nc(-c3ccc4c5c(cccc35)-c3c-4c(-c4ccccc4)c4ccccc4c3-c3ccccc3)n2)cc1 ; c1ccc(-c2nc(-c3ccc(-c4ccnc5ccccc45)cc3)nc(-c3ccc4c5c(cccc35)-c3c-4c(-c4ccccc4)c4ccccc4c3-c3ccccc3)n2)cc1 ; c1ccc(-c2nc(-c3ccc(-c4ccc5c6c(cccc46)-c4c-5c(-c5ccccc5)c5ccccc5c4-c4ccccc4)cc3)nc(-c3cccc(-c4ccncc4)c3)n2)cc1]

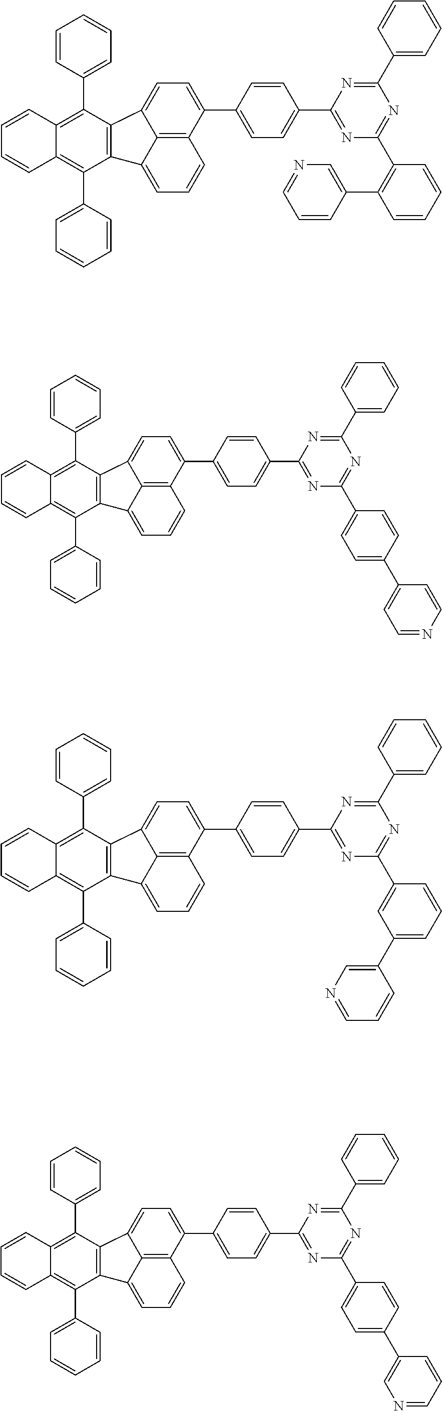 [SMILES: c1ccc(-c2nc(-c3ccc(-c4ccc5c6c(cccc46)-c4c-5c(-c5ccccc5)c5ccccc5c4-c4ccccc4)cc3)nc(-c3ccccc3-c3cccnc3)n2)cc1 ; c1ccc(-c2nc(-c3ccc(-c4ccncc4)cc3)nc(-c3ccc(-c4ccc5c6c(cccc46)-c4c-5c(-c5ccccc5)c5ccccc5c4-c4ccccc4)cc3)n2)cc1 ; c1ccc(-c2nc(-c3ccc(-c4ccc5c6c(cccc46)-c4c-5c(-c5ccccc5)c5ccccc5c4-c4ccccc4)cc3)nc(-c3cccc(-c4cccnc4)c3)n2)cc1 ; c1ccc(-c2nc(-c3ccc(-c4cccnc4)cc3)nc(-c3ccc(-c4ccc5c6c(cccc46)-c4c-5c(-c5ccccc5)c5ccccc5c4-c4ccccc4)cc3)n2)cc1]

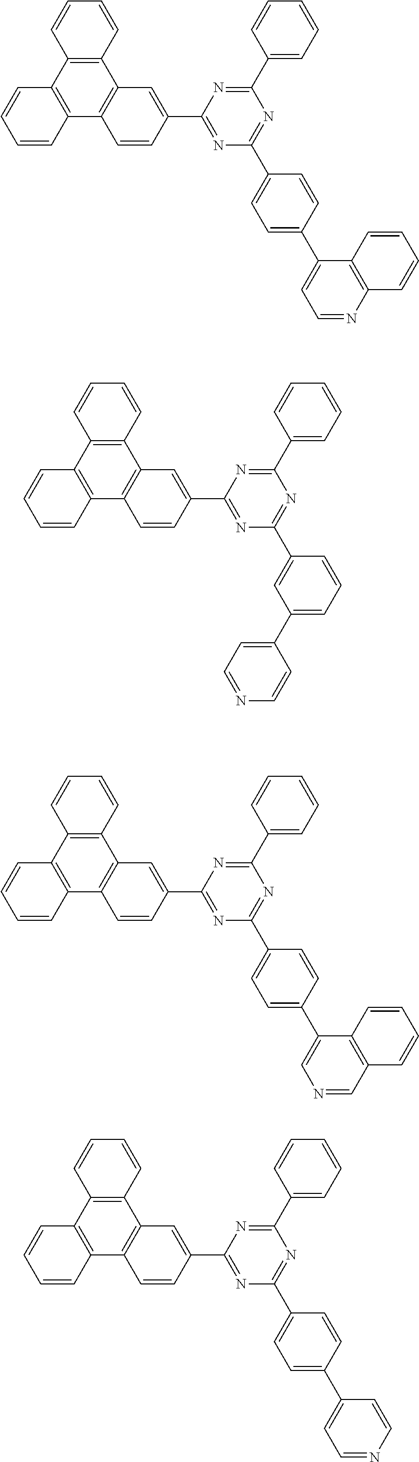

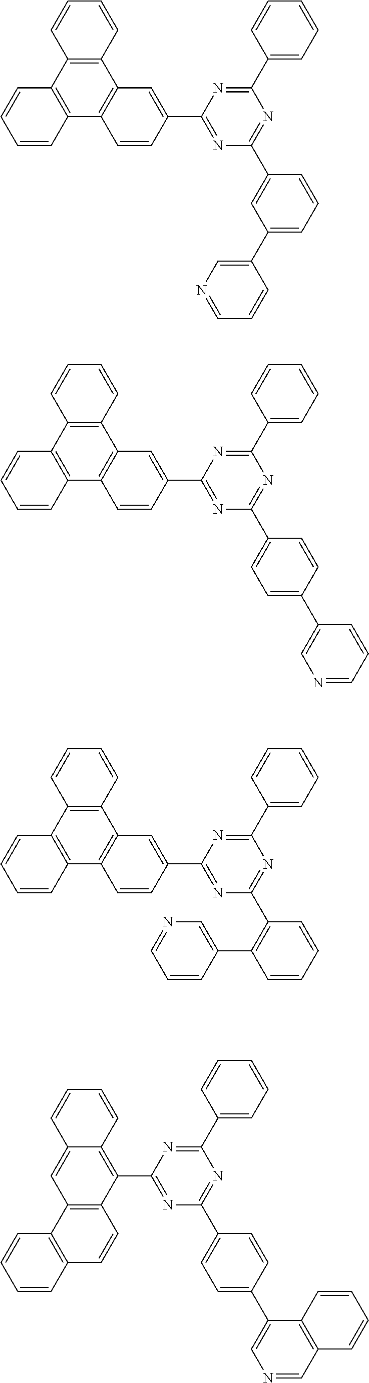 [SMILES: c1ccc(-c2nc(-c3cccc(-c4cccnc4)c3)nc(-c3ccc4c5ccccc5c5ccccc5c4c3)n2)cc1 ; c1ccc(-c2nc(-c3ccc(-c4cccnc4)cc3)nc(-c3ccc4c5ccccc5c5ccccc5c4c3)n2)cc1 ; c1ccc(-c2nc(-c3ccc4c5ccccc5c5ccccc5c4c3)nc(-c3ccccc3-c3cccnc3)n2)cc1 ; c1ccc(-c2nc(-c3ccc(-c4cncc5ccccc45)cc3)nc(-c3c4ccccc4cc4c3ccc3ccccc34)n2)cc1]

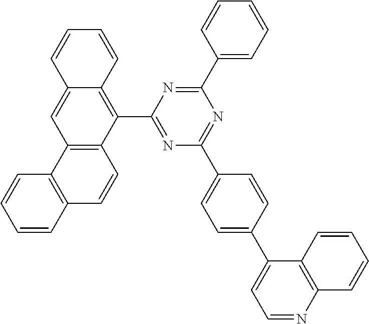 [SMILES: c1ccc(-c2nc(-c3ccc(-c4ccnc5ccccc45)cc3)nc(-c3c4ccccc4cc4c3ccc3ccccc34)n2)cc1]

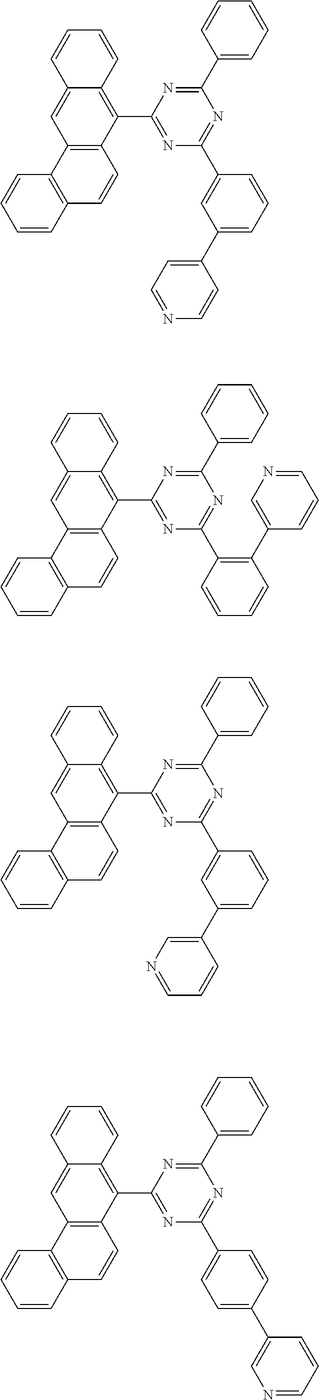 [SMILES: c1ccc(-c2nc(-c3cccc(-c4ccncc4)c3)nc(-c3c4ccccc4cc4c3ccc3ccccc34)n2)cc1 ; c1ccc(-c2nc(-c3ccccc3-c3cccnc3)nc(-c3c4ccccc4cc4c3ccc3ccccc34)n2)cc1 ; c1ccc(-c2nc(-c3cccc(-c4cccnc4)c3)nc(-c3c4ccccc4cc4c3ccc3ccccc34)n2)cc1 ; c1ccc(-c2nc(-c3ccc(-c4cccnc4)cc3)nc(-c3c4ccccc4cc4c3ccc3ccccc34)n2)cc1]

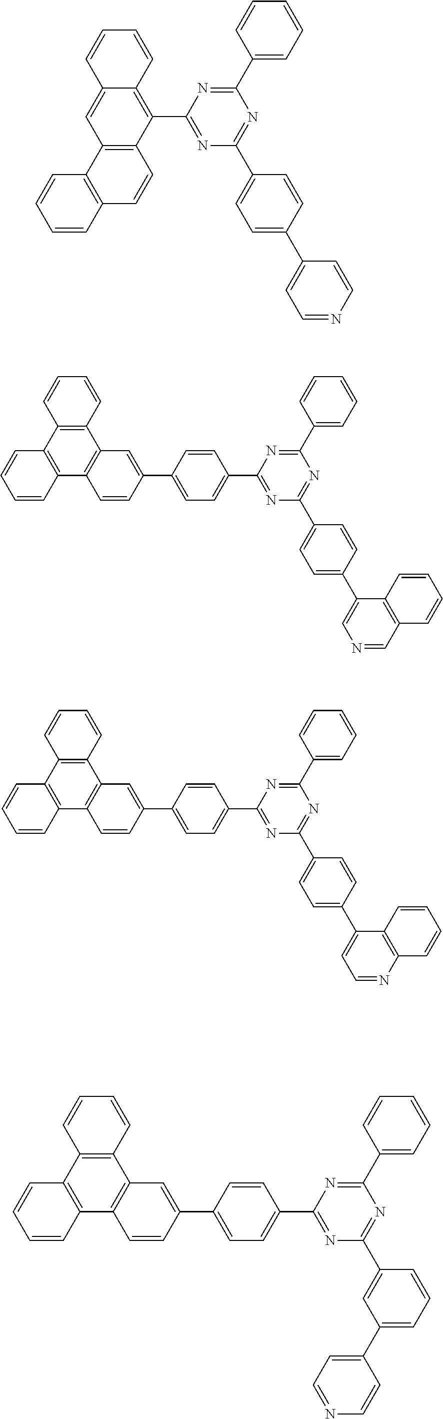 [SMILES: c1ccc(-c2nc(-c3ccc(-c4ccncc4)cc3)nc(-c3c4ccccc4cc4c3ccc3ccccc34)n2)cc1 ; c1ccc(-c2nc(-c3ccc(-c4ccc5c6ccccc6c6ccccc6c5c4)cc3)nc(-c3ccc(-c4cncc5ccccc45)cc3)n2)cc1 ; c1ccc(-c2nc(-c3ccc(-c4ccc5c6ccccc6c6ccccc6c5c4)cc3)nc(-c3ccc(-c4ccnc5ccccc45)cc3)n2)cc1 ; c1ccc(-c2nc(-c3ccc(-c4ccc5c6ccccc6c6ccccc6c5c4)cc3)nc(-c3cccc(-c4ccncc4)c3)n2)cc1]

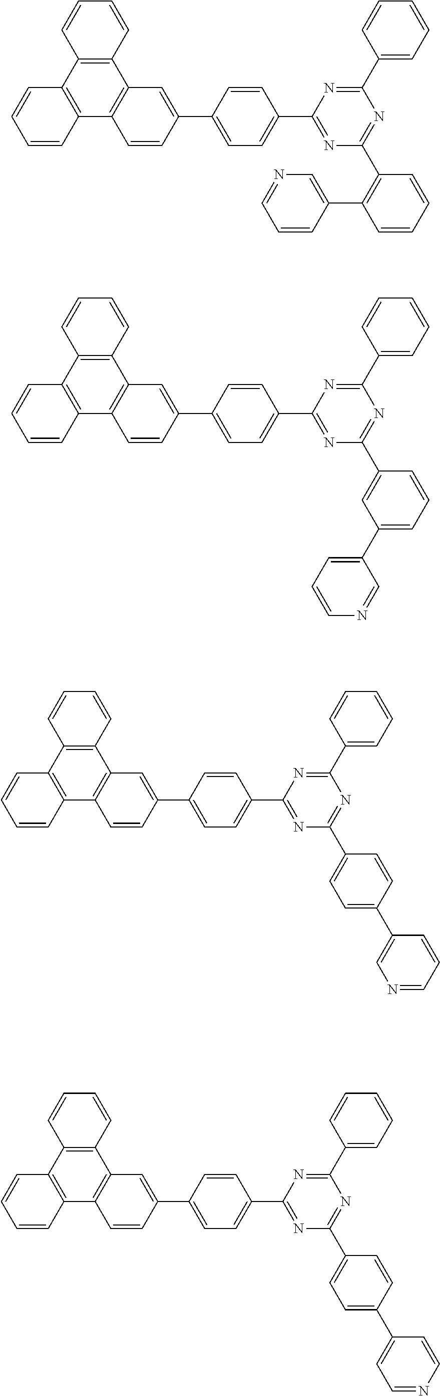 [SMILES: c1ccc(-c2nc(-c3ccc(-c4ccc5c6ccccc6c6ccccc6c5c4)cc3)nc(-c3ccccc3-c3cccnc3)n2)cc1 ; c1ccc(-c2nc(-c3ccc(-c4ccc5c6ccccc6c6ccccc6c5c4)cc3)nc(-c3cccc(-c4cccnc4)c3)n2)cc1 ; c1ccc(-c2nc(-c3ccc(-c4cccnc4)cc3)nc(-c3ccc(-c4ccc5c6ccccc6c6ccccc6c5c4)cc3)n2)cc1 ; c1ccc(-c2nc(-c3ccc(-c4ccncc4)cc3)nc(-c3ccc(-c4ccc5c6ccccc6c6ccccc6c5c4)cc3)n2)cc1]

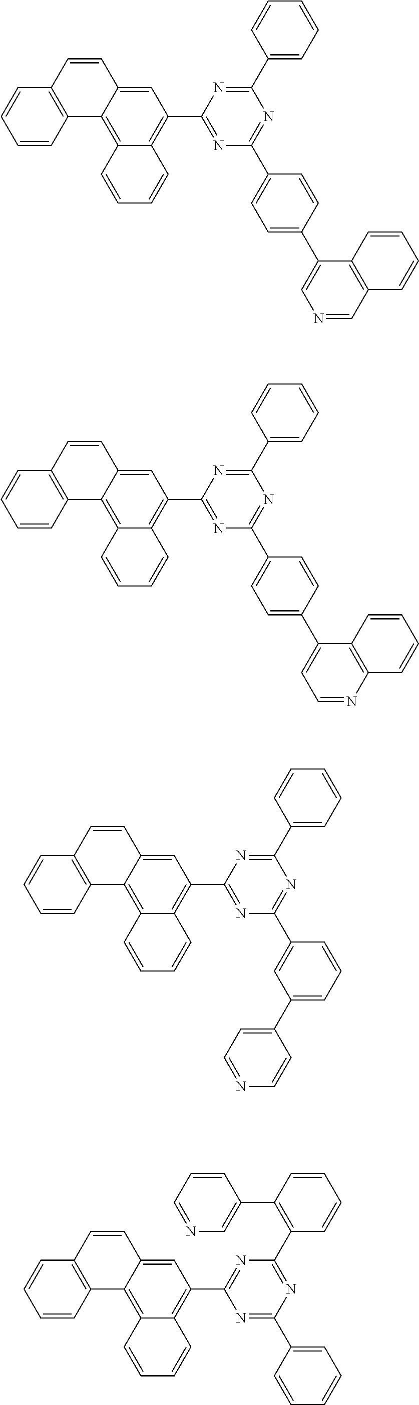 [SMILES: c1ccc(-c2nc(-c3ccc(-c4cncc5ccccc45)cc3)nc(-c3cc4ccc5ccccc5c4c4ccccc34)n2)cc1 ; c1ccc(-c2nc(-c3ccc(-c4ccnc5ccccc45)cc3)nc(-c3cc4ccc5ccccc5c4c4ccccc34)n2)cc1 ; c1ccc(-c2nc(-c3cccc(-c4ccncc4)c3)nc(-c3cc4ccc5ccccc5c4c4ccccc34)n2)cc1 ; c1ccc(-c2nc(-c3ccccc3-c3cccnc3)nc(-c3cc4ccc5ccccc5c4c4ccccc34)n2)cc1]

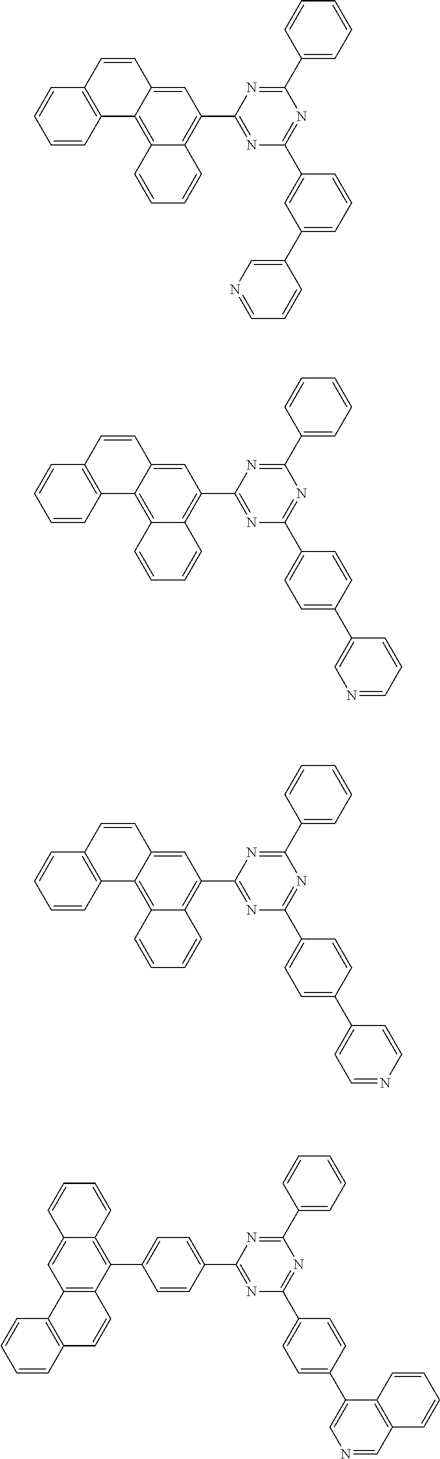 [SMILES: c1ccc(-c2nc(-c3cccc(-c4cccnc4)c3)nc(-c3cc4ccc5ccccc5c4c4ccccc34)n2)cc1 ; c1ccc(-c2nc(-c3ccc(-c4cccnc4)cc3)nc(-c3cc4ccc5ccccc5c4c4ccccc34)n2)cc1 ; c1ccc(-c2nc(-c3ccc(-c4ccncc4)cc3)nc(-c3cc4ccc5ccccc5c4c4ccccc34)n2)cc1 ; c1ccc(-c2nc(-c3ccc(-c4cncc5ccccc45)cc3)nc(-c3ccc(-c4c5ccccc5cc5c4ccc4ccccc45)cc3)n2)cc1]

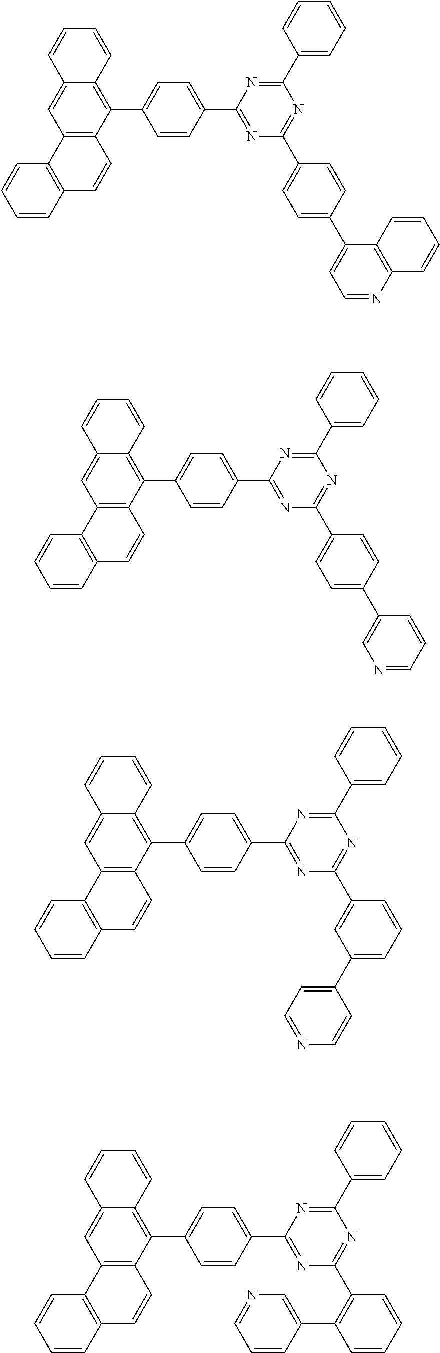 [SMILES: c1ccc(-c2nc(-c3ccc(-c4ccnc5ccccc45)cc3)nc(-c3ccc(-c4c5ccccc5cc5c4ccc4ccccc45)cc3)n2)cc1 ; c1ccc(-c2nc(-c3ccc(-c4cccnc4)cc3)nc(-c3ccc(-c4c5ccccc5cc5c4ccc4ccccc45)cc3)n2)cc1 ; c1ccc(-c2nc(-c3ccc(-c4c5ccccc5cc5c4ccc4ccccc45)cc3)nc(-c3cccc(-c4ccncc4)c3)n2)cc1 ; c1ccc(-c2nc(-c3ccc(-c4c5ccccc5cc5c4ccc4ccccc45)cc3)nc(-c3ccccc3-c3cccnc3)n2)cc1]

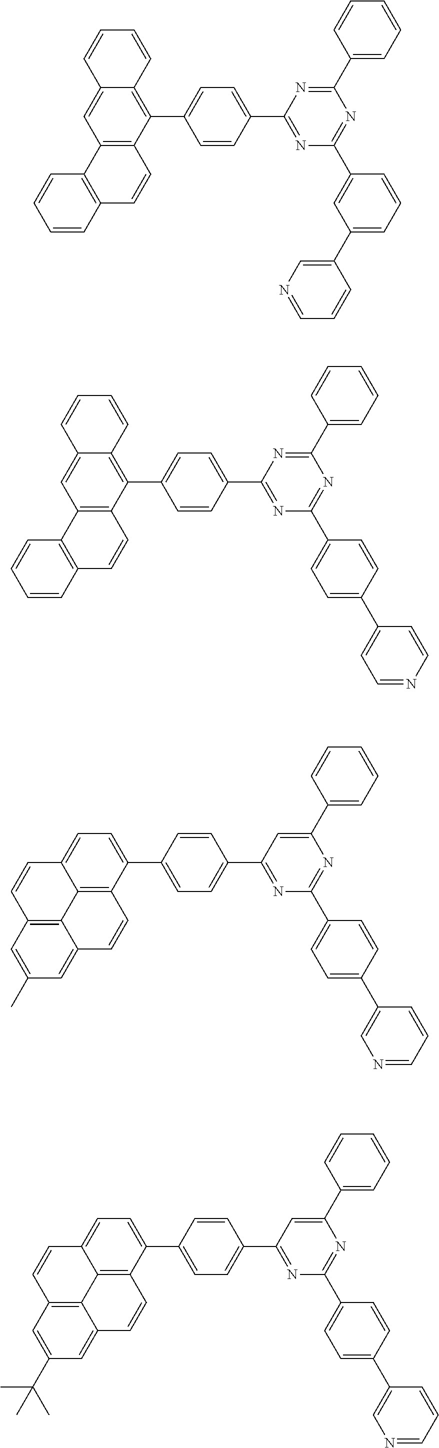 [SMILES: c1ccc(-c2nc(-c3ccc(-c4c5ccccc5cc5c4ccc4ccccc45)cc3)nc(-c3cccc(-c4cccnc4)c3)n2)cc1 ; c1ccc(-c2nc(-c3ccc(-c4ccncc4)cc3)nc(-c3ccc(-c4c5ccccc5cc5c4ccc4ccccc45)cc3)n2)cc1 ; Cc1cc2ccc3ccc(-c4ccc(-c5cc(-c6ccccc6)nc(-c6ccc(-c7cccnc7)cc6)n5)cc4)c4ccc(c1)c2c34 ; CC(C)(C)c1cc2ccc3ccc(-c4ccc(-c5cc(-c6ccccc6)nc(-c6ccc(-c7cccnc7)cc6)n5)cc4)c4ccc(c1)c2c34]

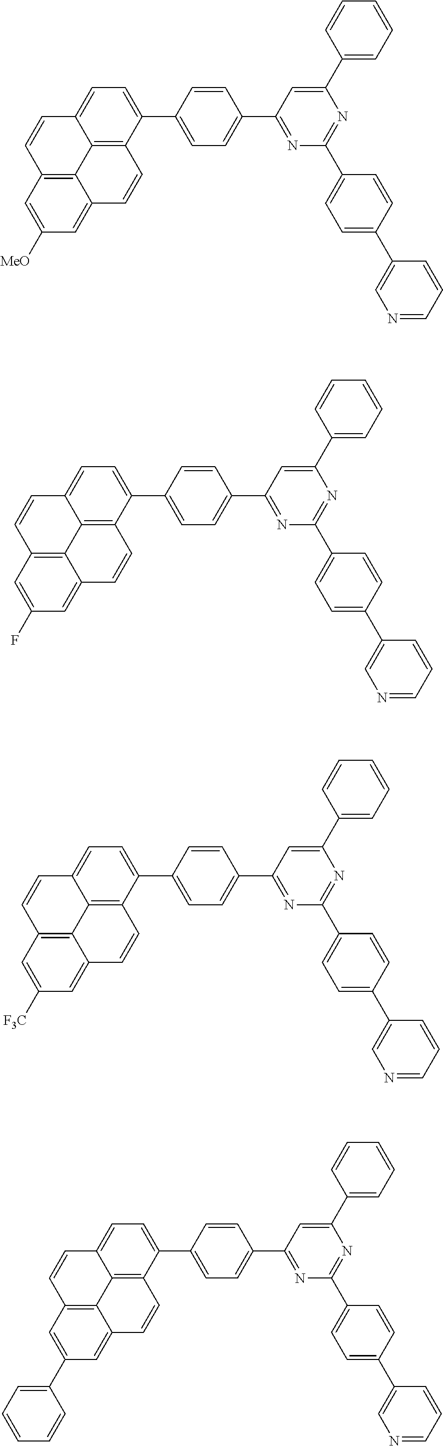 [SMILES: COc1cc2ccc3ccc(-c4ccc(-c5cc(-c6ccccc6)nc(-c6ccc(-c7cccnc7)cc6)n5)cc4)c4ccc(c1)c2c34 ; Fc1cc2ccc3ccc(-c4ccc(-c5cc(-c6ccccc6)nc(-c6ccc(-c7cccnc7)cc6)n5)cc4)c4ccc(c1)c2c34 ; FC(F)(F)c1cc2ccc3ccc(-c4ccc(-c5cc(-c6ccccc6)nc(-c6ccc(-c7cccnc7)cc6)n5)cc4)c4ccc(c1)c2c34 ; c1ccc(-c2cc3ccc4ccc(-c5ccc(-c6cc(-c7ccccc7)nc(-c7ccc(-c8cccnc8)cc7)n6)cc5)c5ccc(c2)c3c45)cc1]

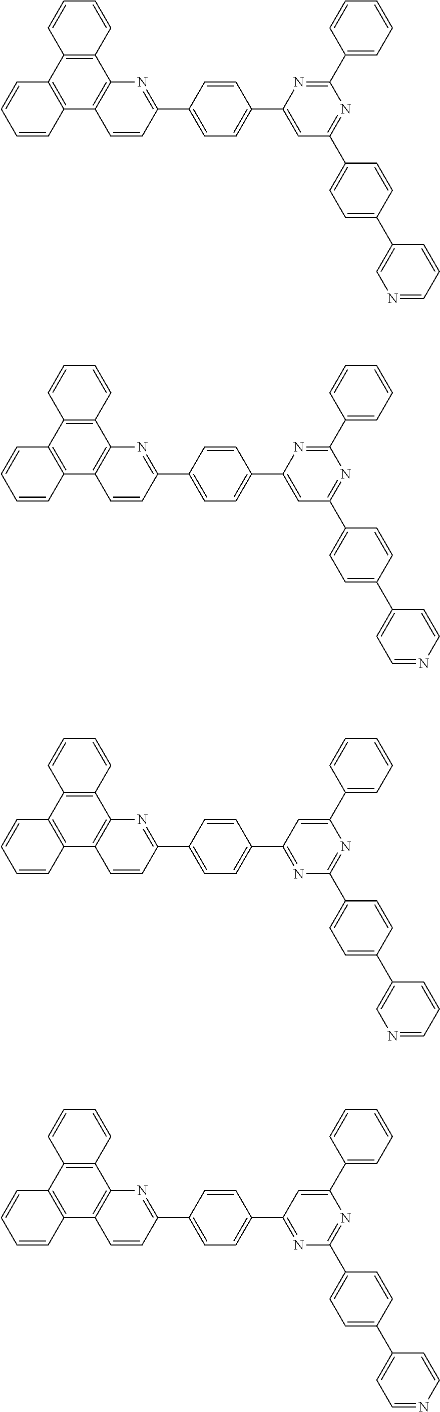 [SMILES: c1ccc(-c2nc(-c3ccc(-c4cccnc4)cc3)cc(-c3ccc(-c4ccc5c6ccccc6c6ccccc6c5n4)cc3)n2)cc1 ; c1ccc(-c2nc(-c3ccc(-c4ccncc4)cc3)cc(-c3ccc(-c4ccc5c6ccccc6c6ccccc6c5n4)cc3)n2)cc1 ; c1ccc(-c2cc(-c3ccc(-c4ccc5c6ccccc6c6ccccc6c5n4)cc3)nc(-c3ccc(-c4cccnc4)cc3)n2)cc1 ; c1ccc(-c2cc(-c3ccc(-c4ccc5c6ccccc6c6ccccc6c5n4)cc3)nc(-c3ccc(-c4ccncc4)cc3)n2)cc1]

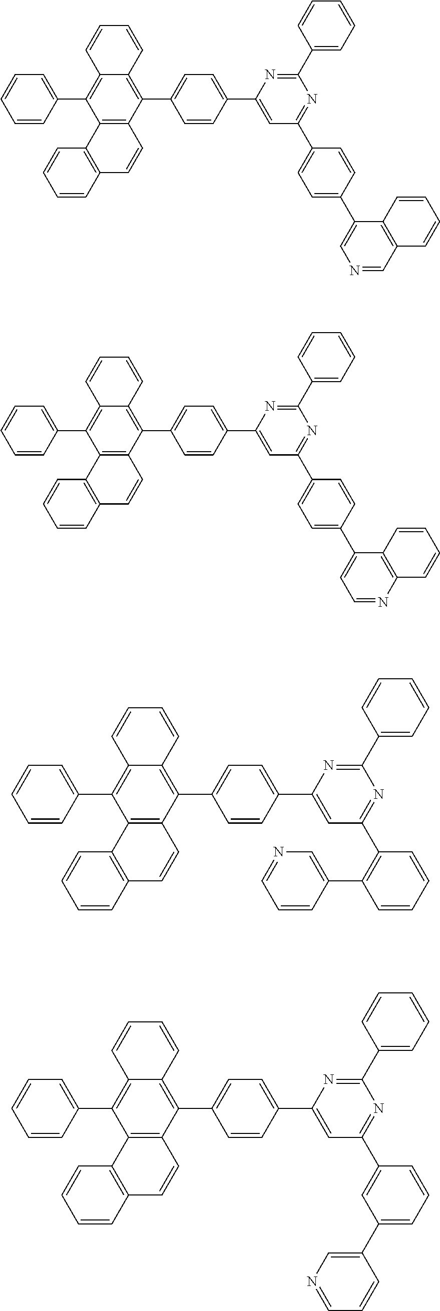 [SMILES: c1ccc(-c2nc(-c3ccc(-c4cncc5ccccc45)cc3)cc(-c3ccc(-c4c5ccccc5c(-c5ccccc5)c5c4ccc4ccccc45)cc3)n2)cc1 ; c1ccc(-c2nc(-c3ccc(-c4ccnc5ccccc45)cc3)cc(-c3ccc(-c4c5ccccc5c(-c5ccccc5)c5c4ccc4ccccc45)cc3)n2)cc1 ; c1ccc(-c2nc(-c3ccc(-c4c5ccccc5c(-c5ccccc5)c5c4ccc4ccccc45)cc3)cc(-c3ccccc3-c3cccnc3)n2)cc1 ; c1ccc(-c2nc(-c3ccc(-c4c5ccccc5c(-c5ccccc5)c5c4ccc4ccccc45)cc3)cc(-c3cccc(-c4cccnc4)c3)n2)cc1]

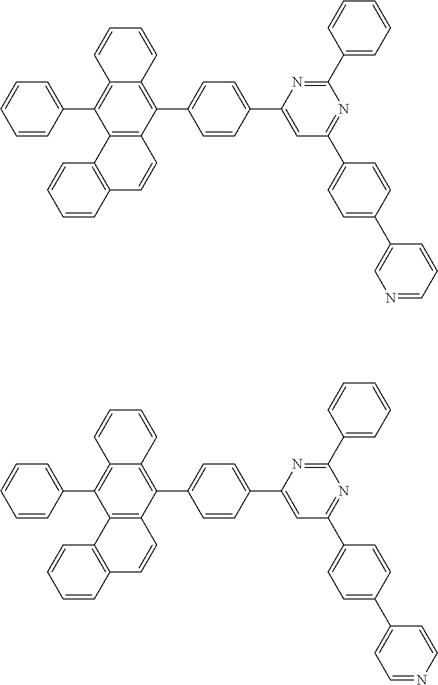 [SMILES: c1ccc(-c2nc(-c3ccc(-c4cccnc4)cc3)cc(-c3ccc(-c4c5ccccc5c(-c5ccccc5)c5c4ccc4ccccc45)cc3)n2)cc1 ; c1ccc(-c2nc(-c3ccc(-c4ccncc4)cc3)cc(-c3ccc(-c4c5ccccc5c(-c5ccccc5)c5c4ccc4ccccc45)cc3)n2)cc1]

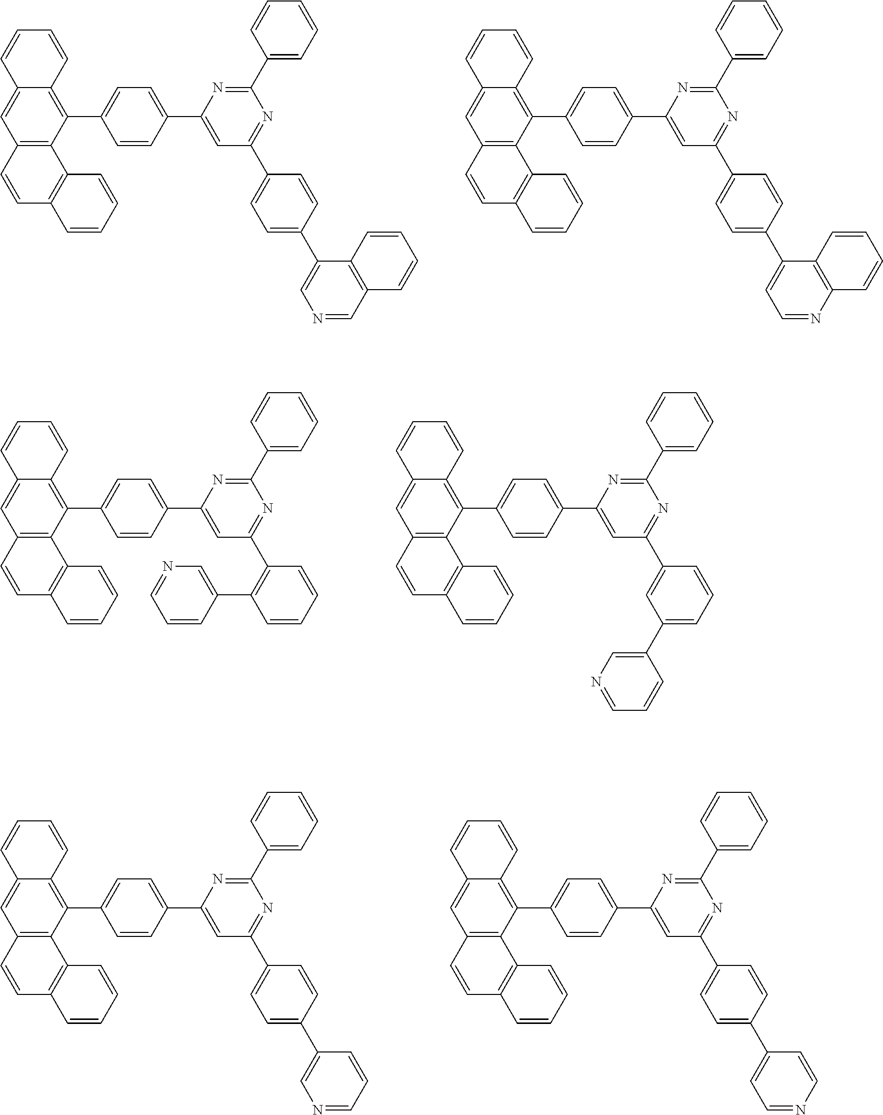

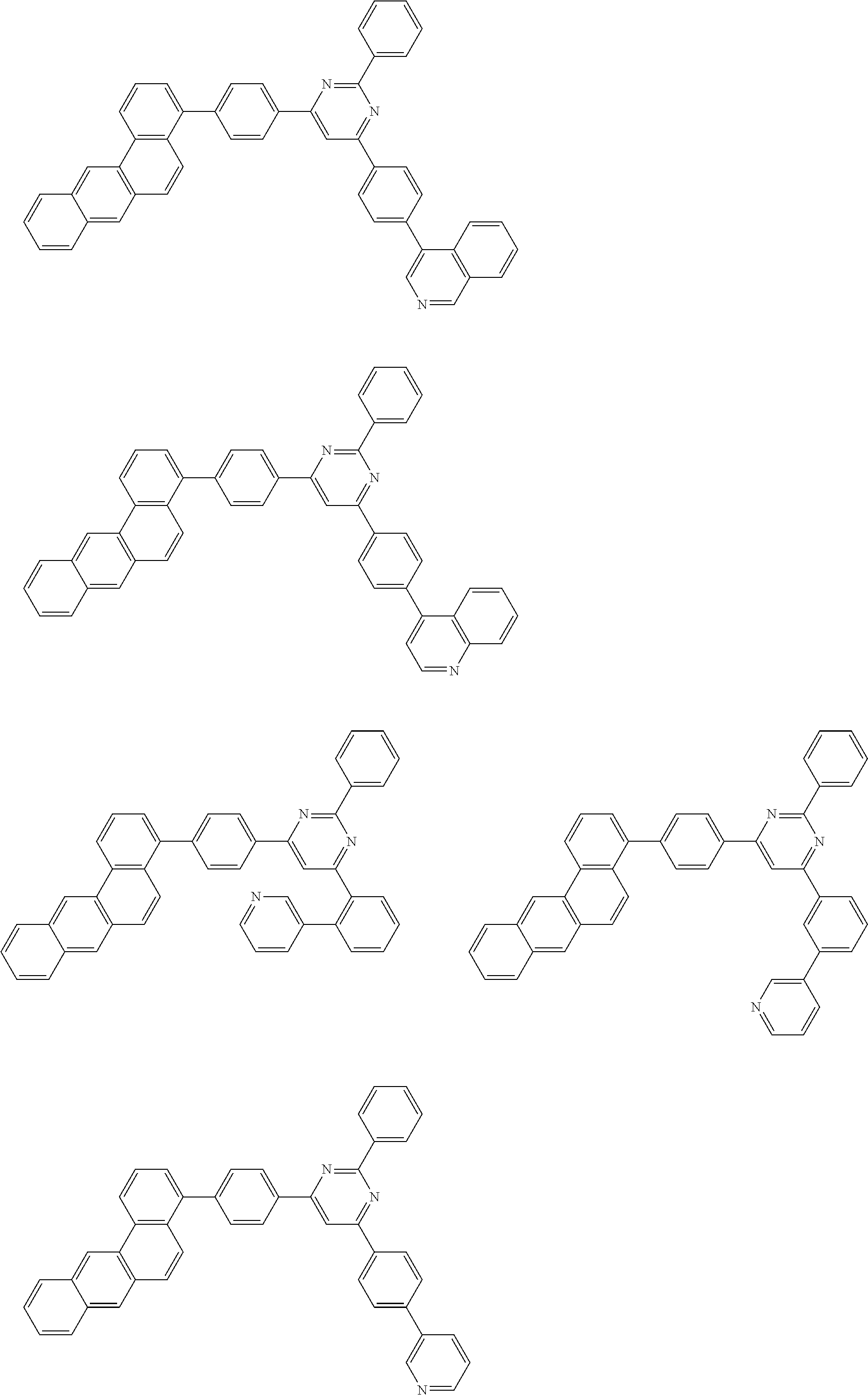 [SMILES: c1ccc(-c2nc(-c3ccc(-c4cncc5ccccc45)cc3)cc(-c3ccc(-c4cccc5c4ccc4cc6ccccc6cc45)cc3)n2)cc1 ; c1ccc(-c2nc(-c3ccc(-c4ccnc5ccccc45)cc3)cc(-c3ccc(-c4cccc5c4ccc4cc6ccccc6cc45)cc3)n2)cc1 ; c1ccc(-c2nc(-c3ccc(-c4cccc5c4ccc4cc6ccccc6cc45)cc3)cc(-c3ccccc3-c3cccnc3)n2)cc1 ; c1ccc(-c2nc(-c3ccc(-c4cccc5c4ccc4cc6ccccc6cc45)cc3)cc(-c3cccc(-c4cccnc4)c3)n2)cc1 ; c1ccc(-c2nc(-c3ccc(-c4cccnc4)cc3)cc(-c3ccc(-c4cccc5c4ccc4cc6ccccc6cc45)cc3)n2)cc1]

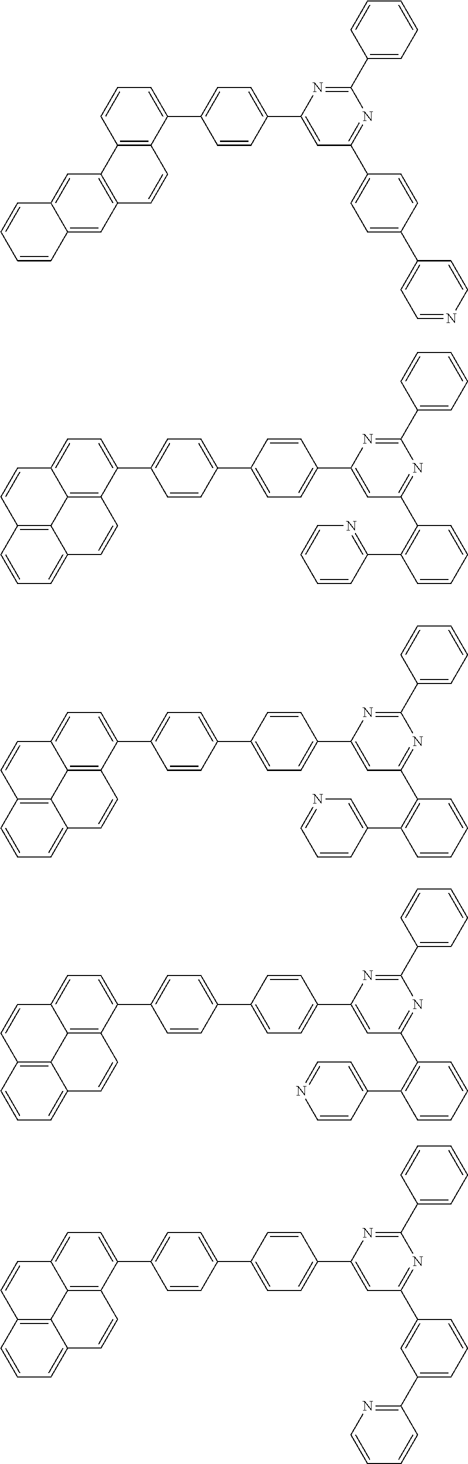

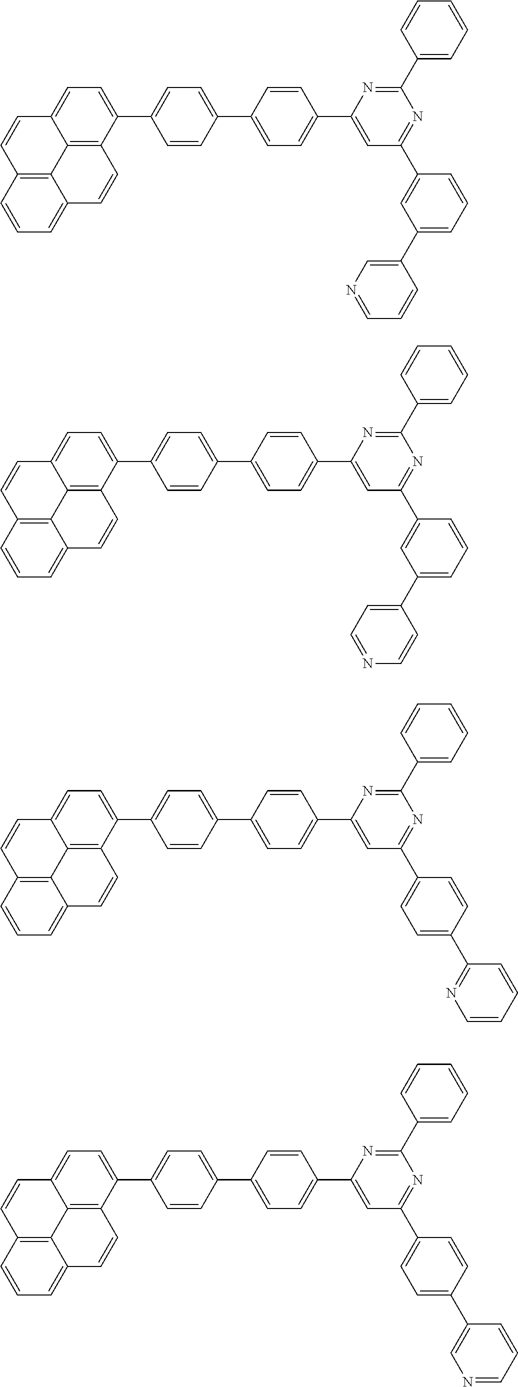

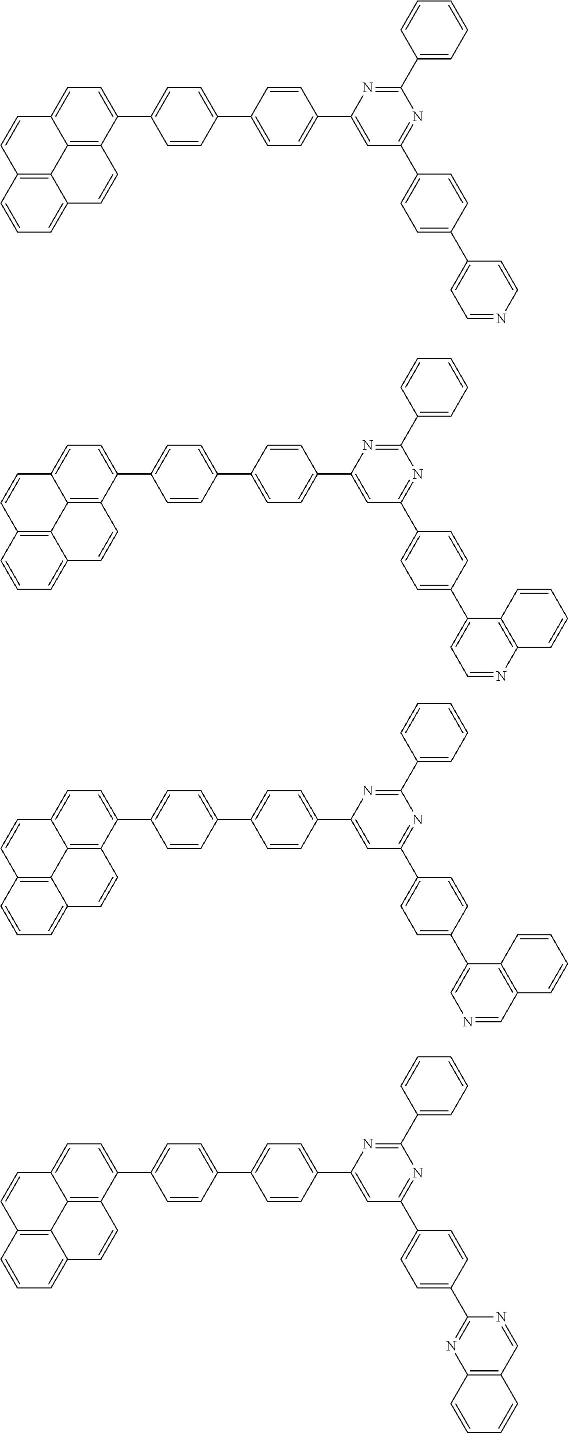

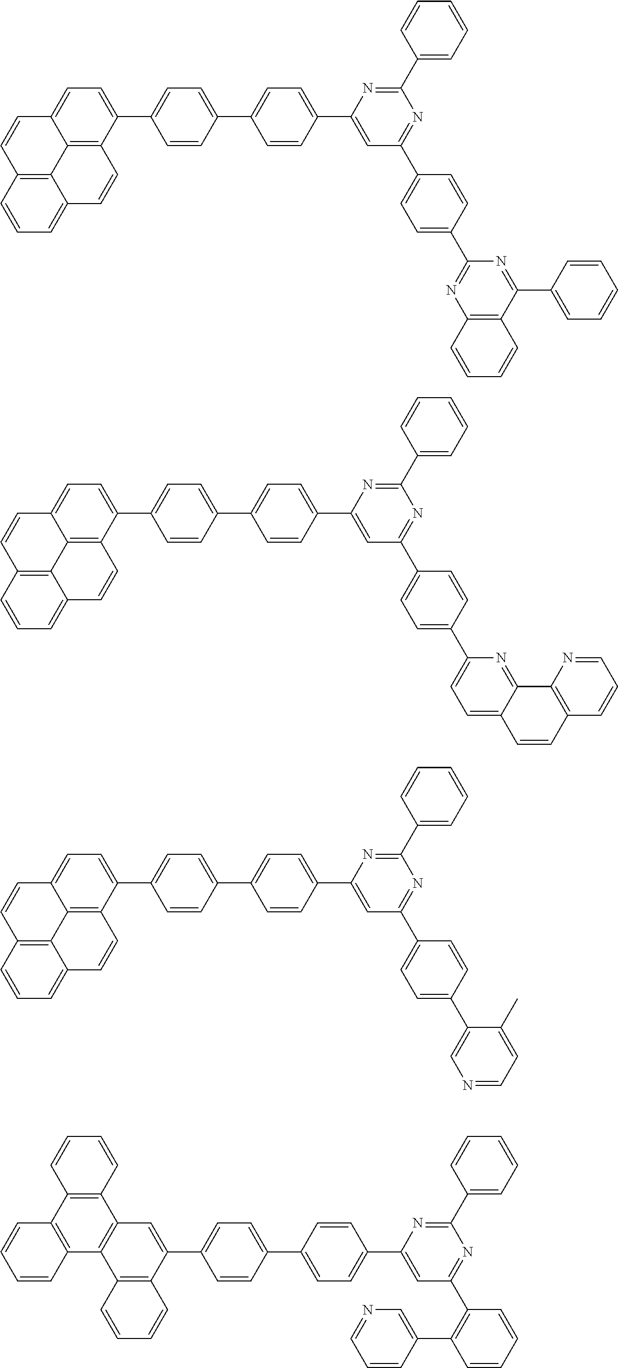

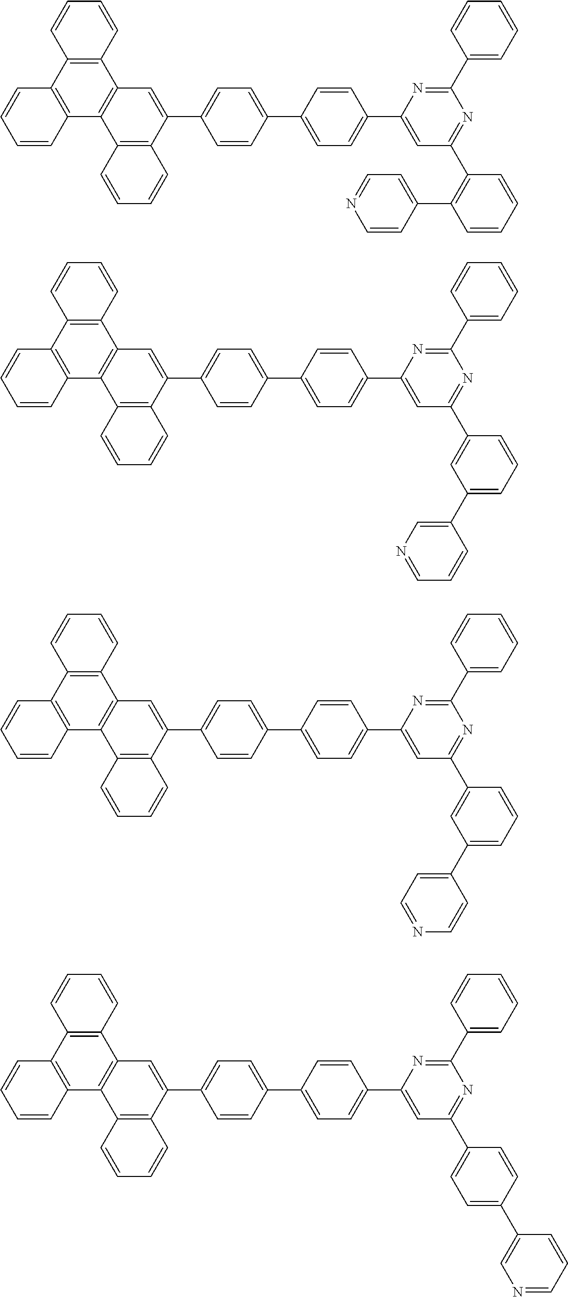

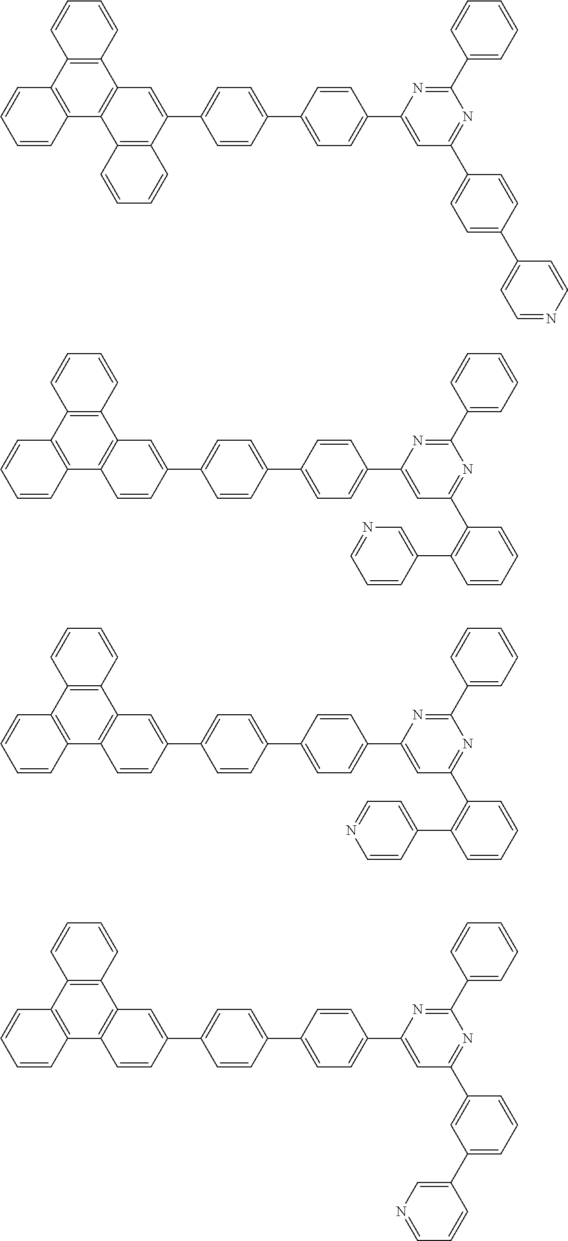

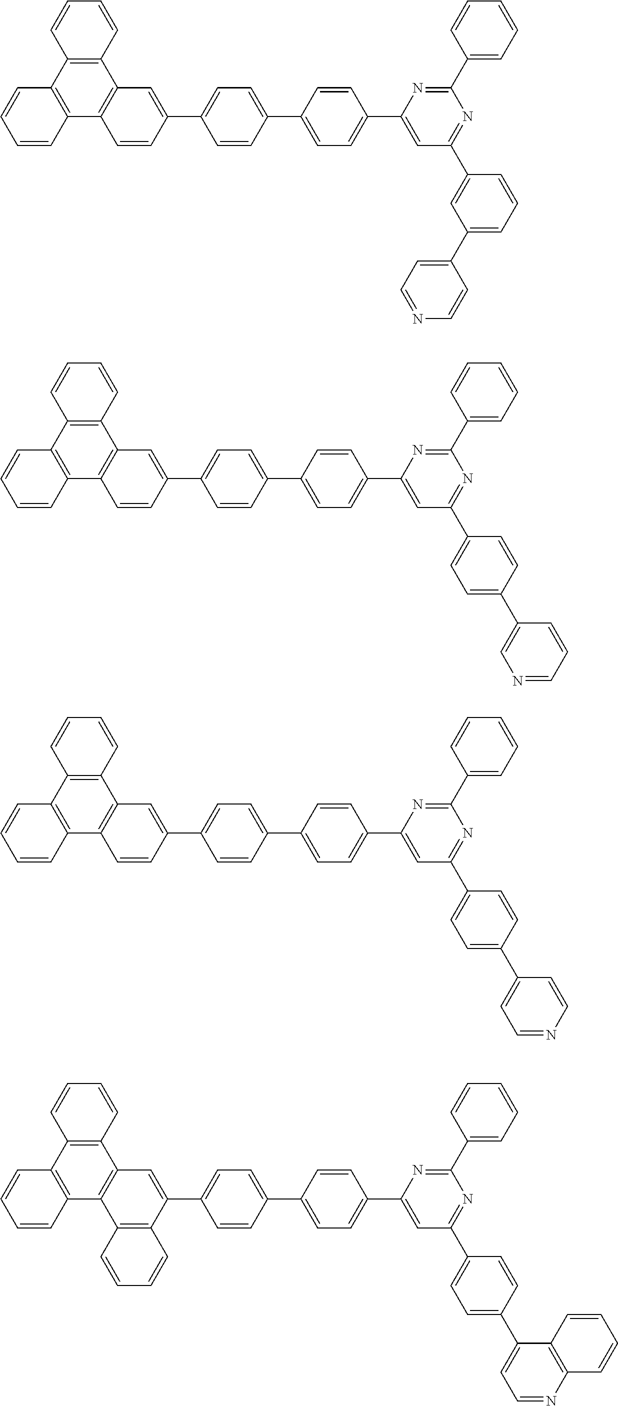

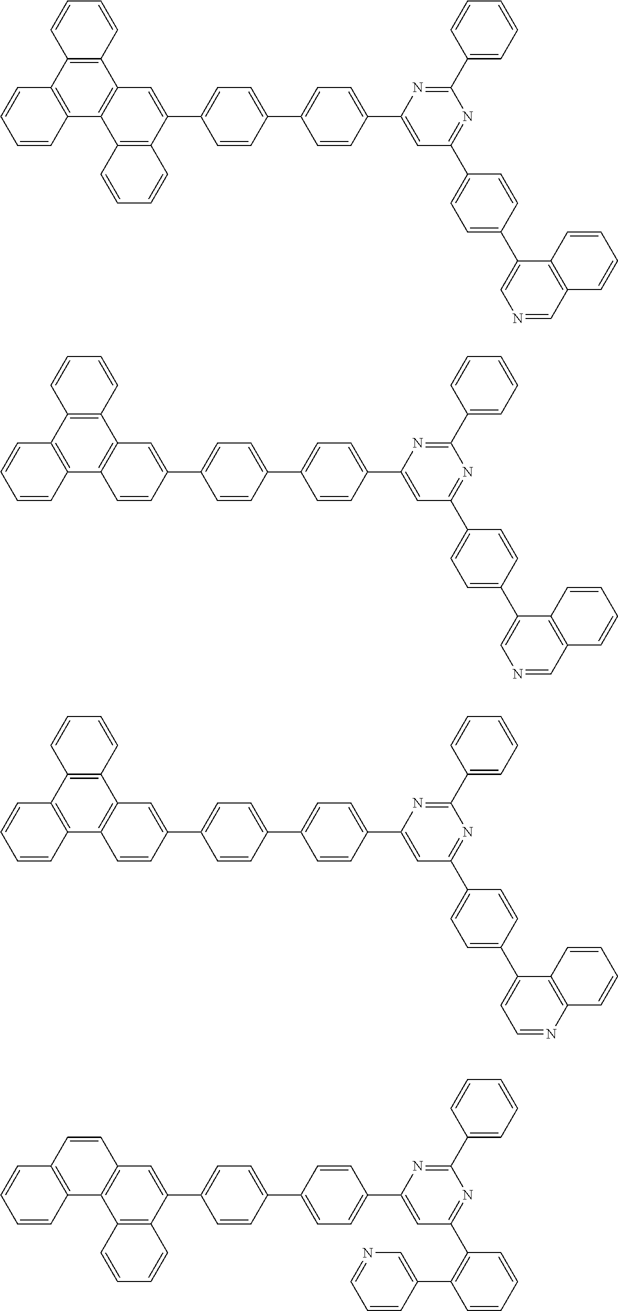

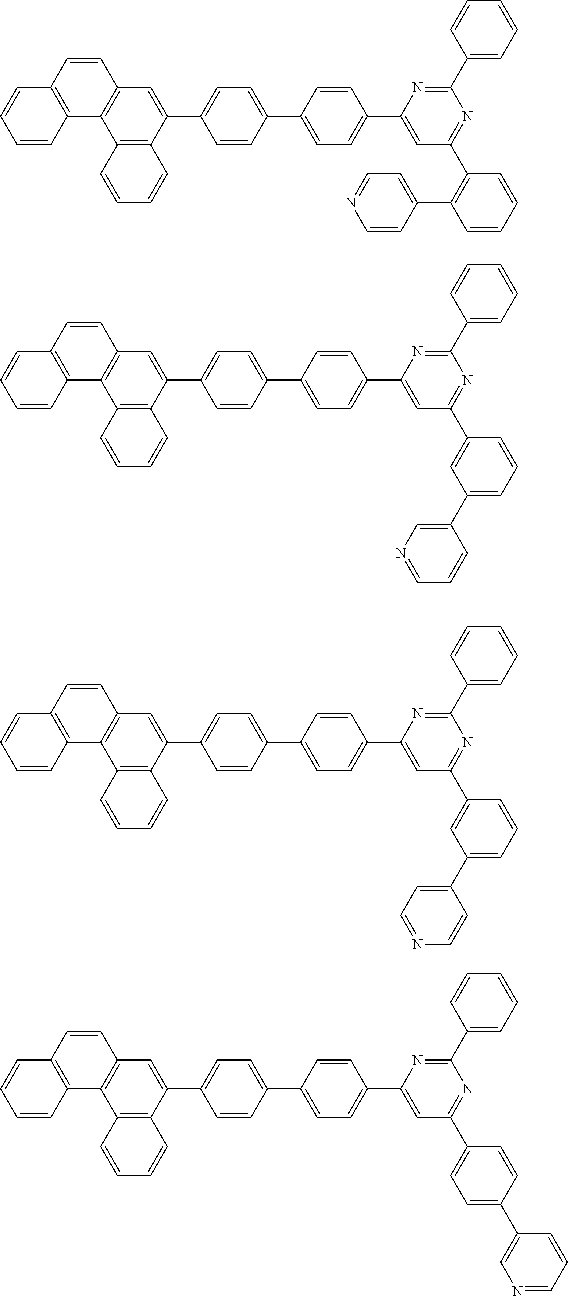

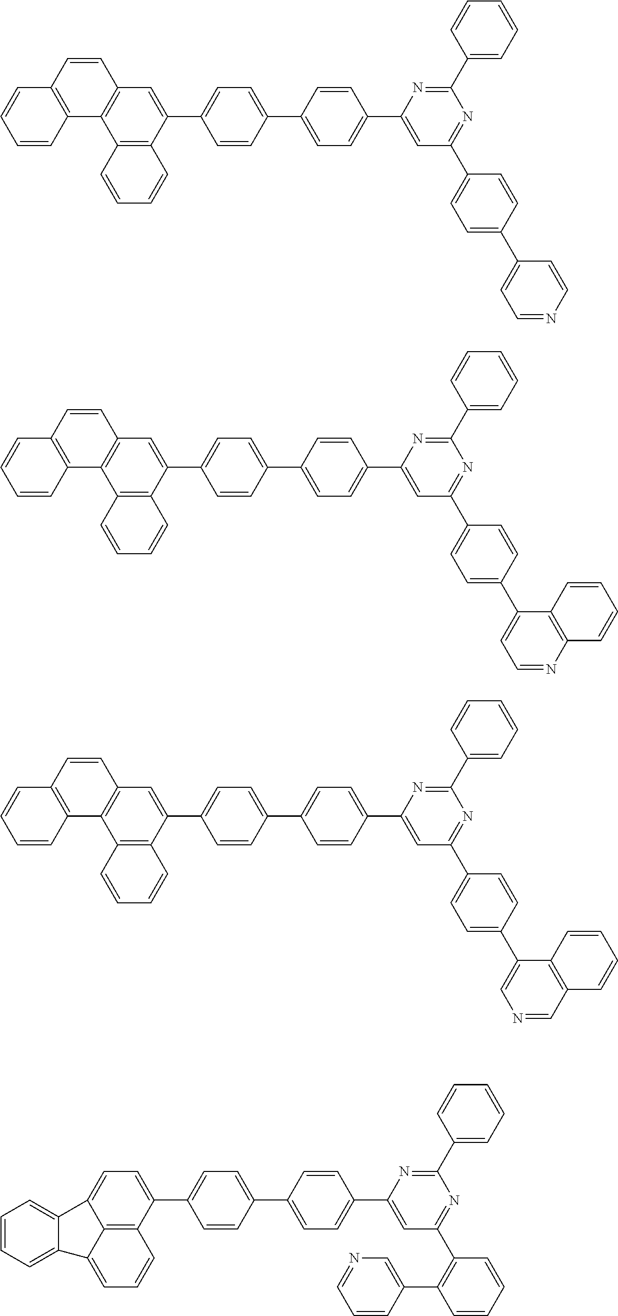

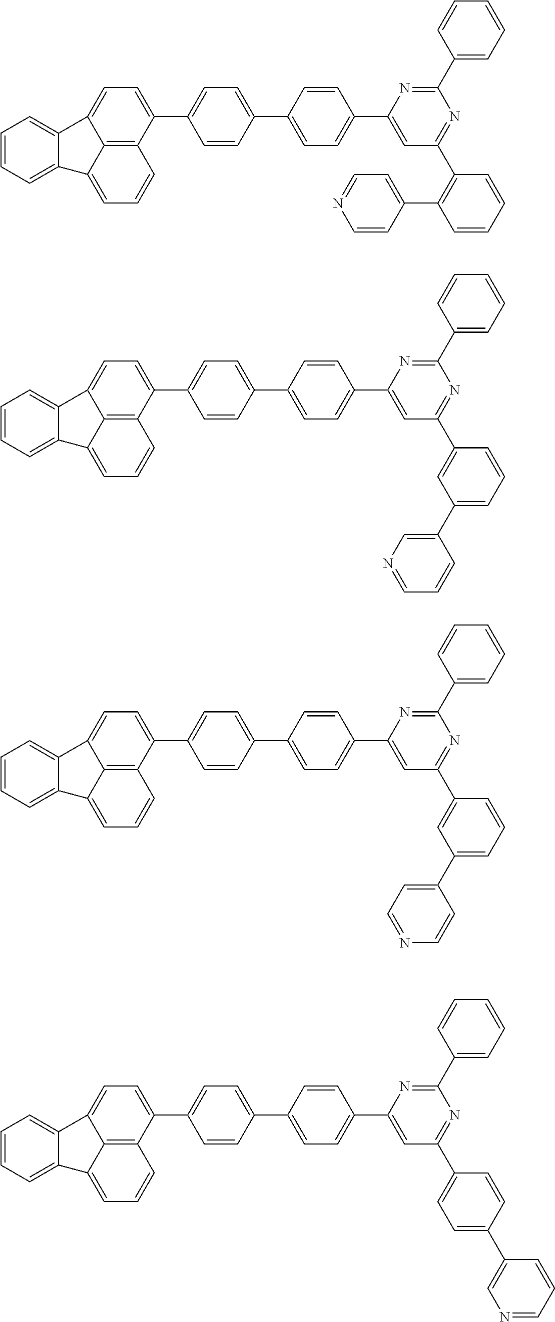

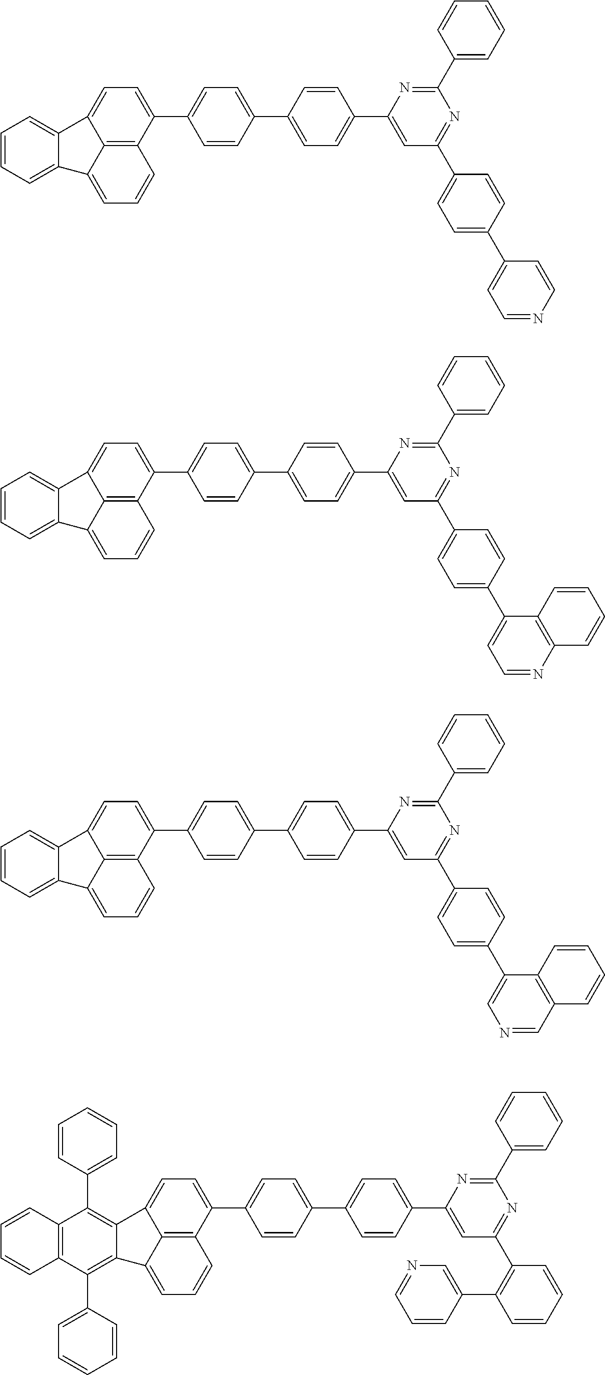

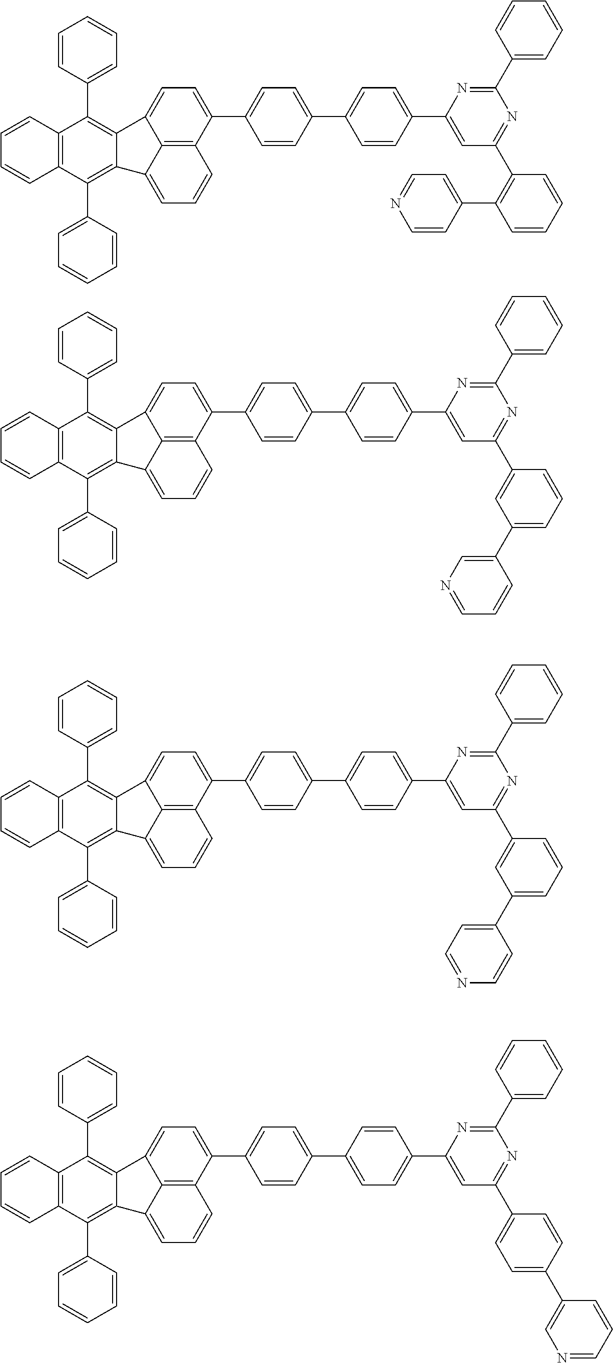

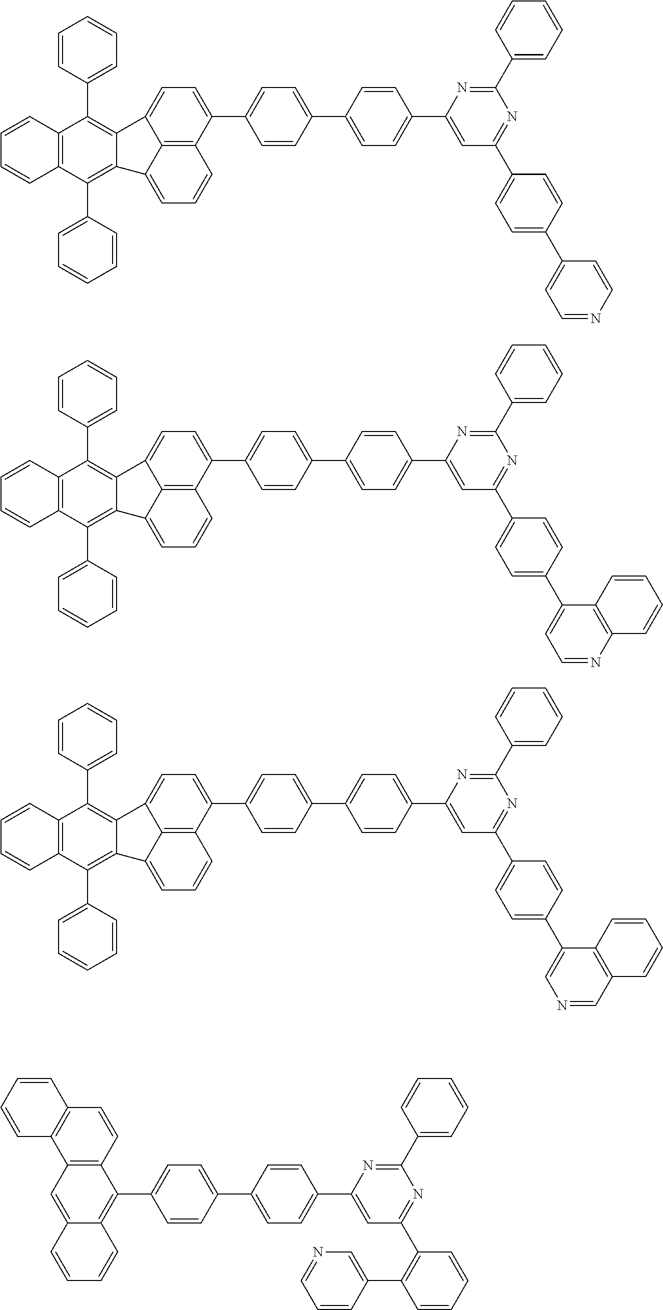

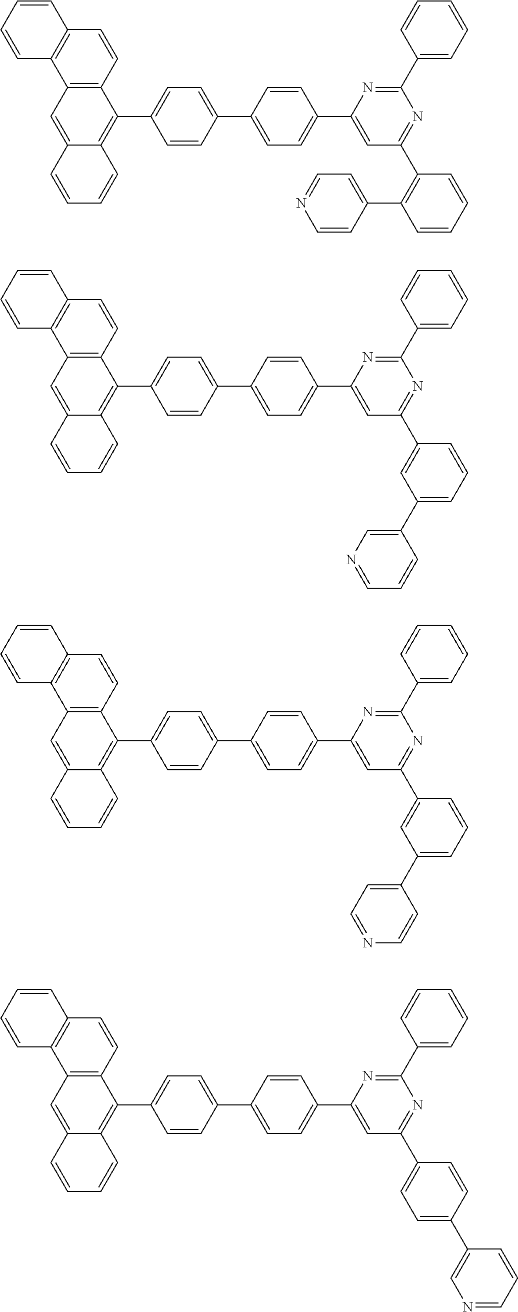

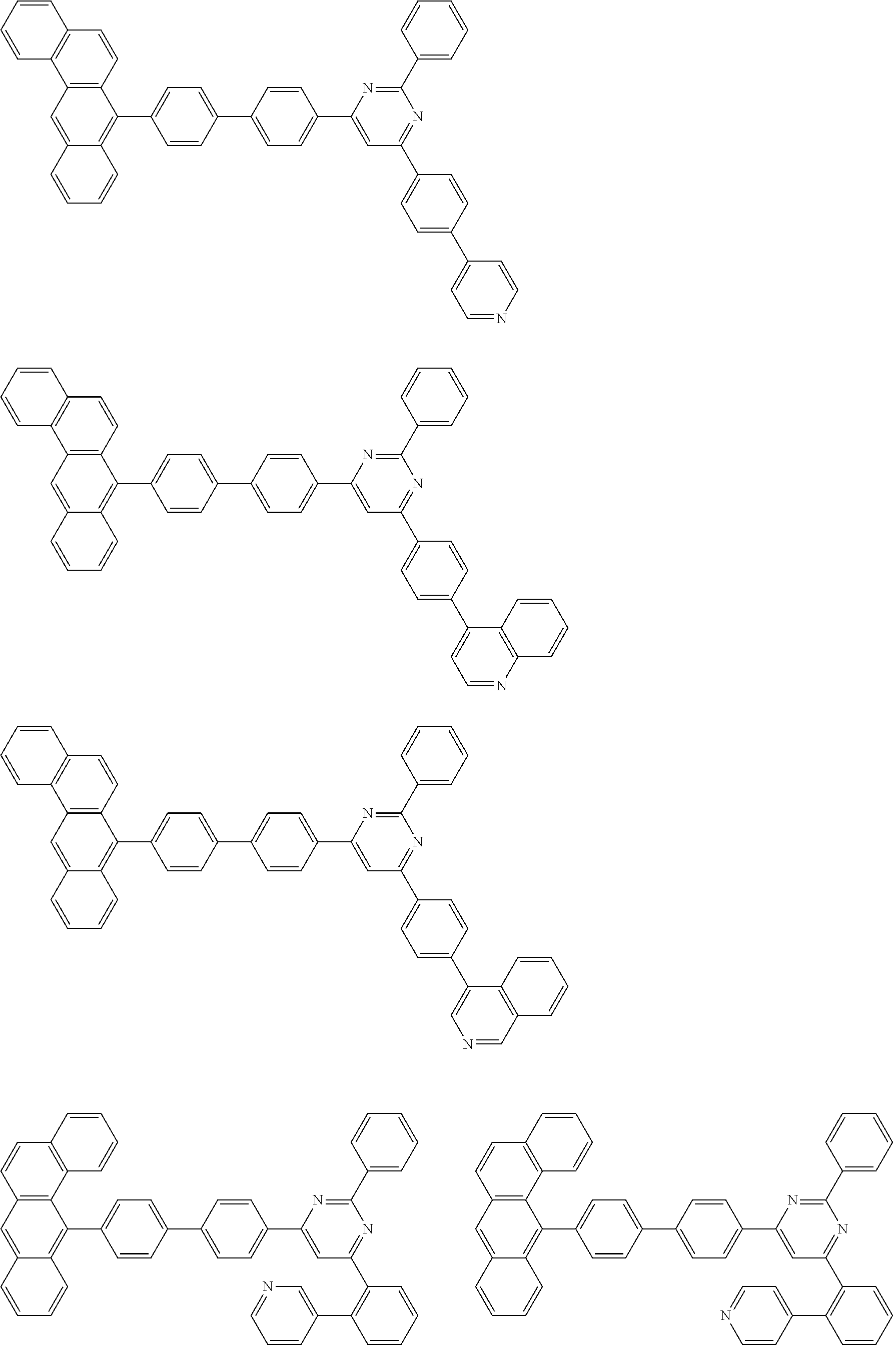

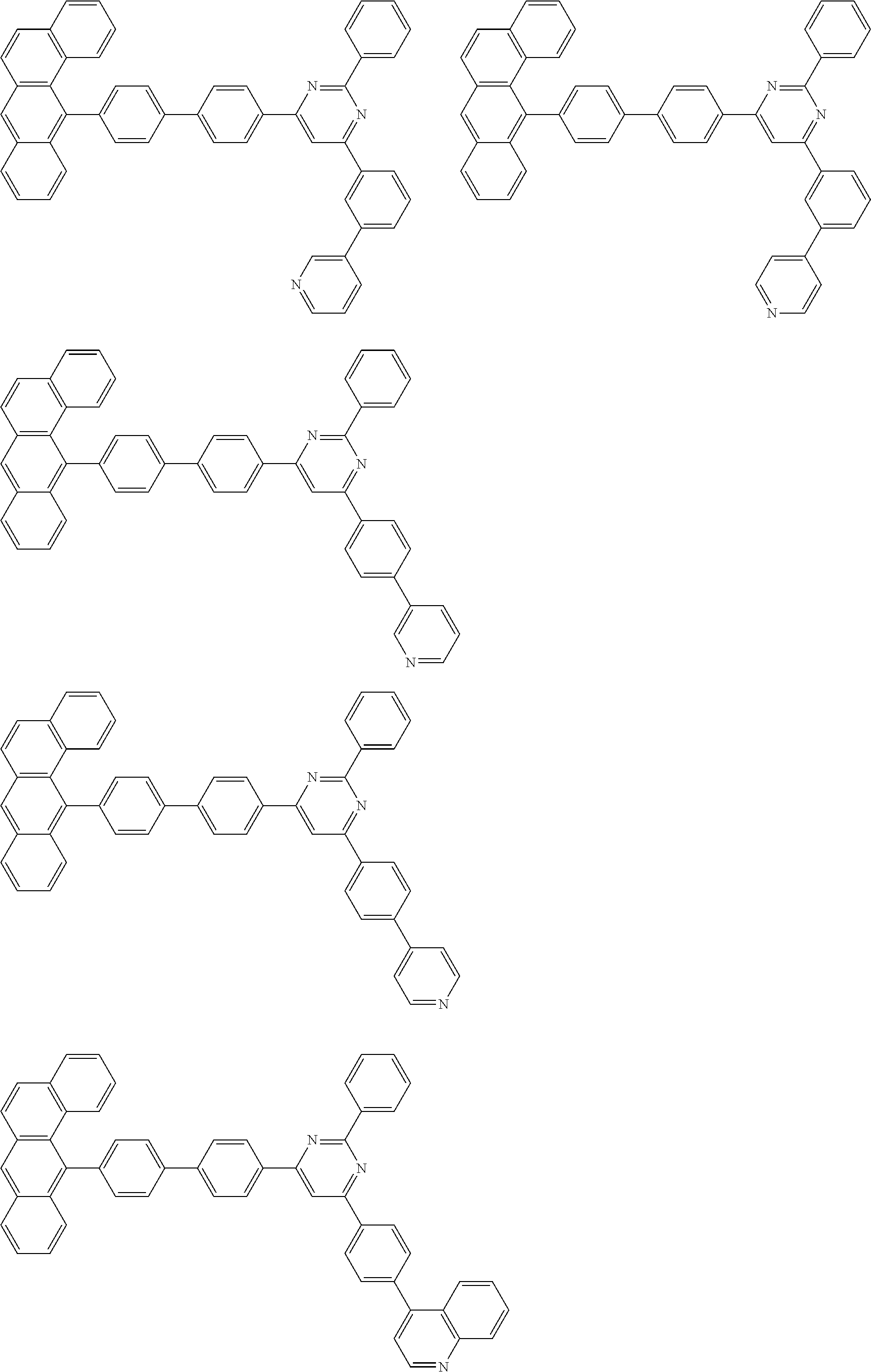

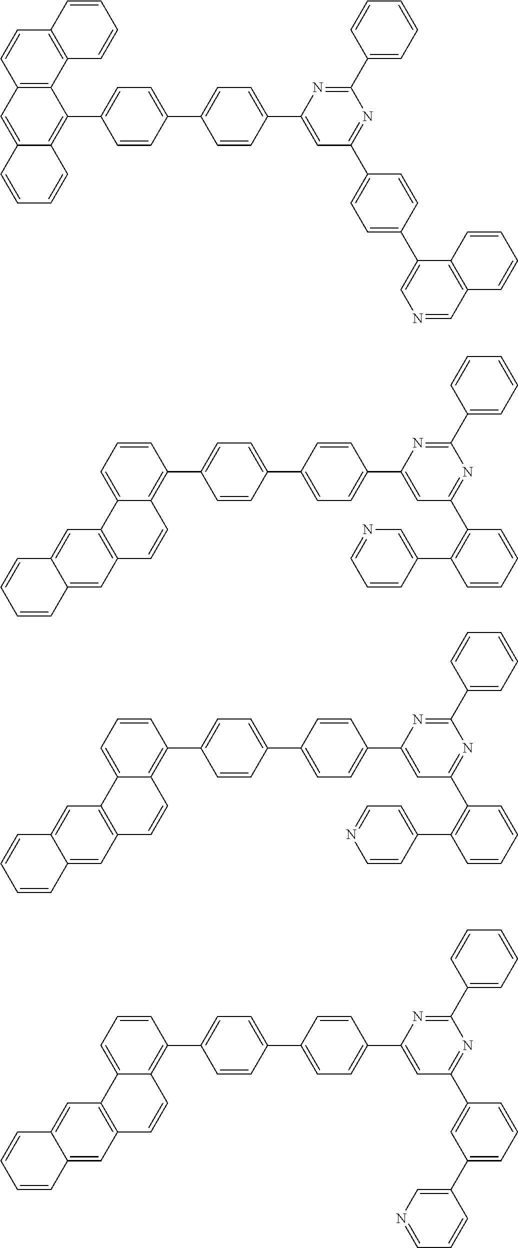

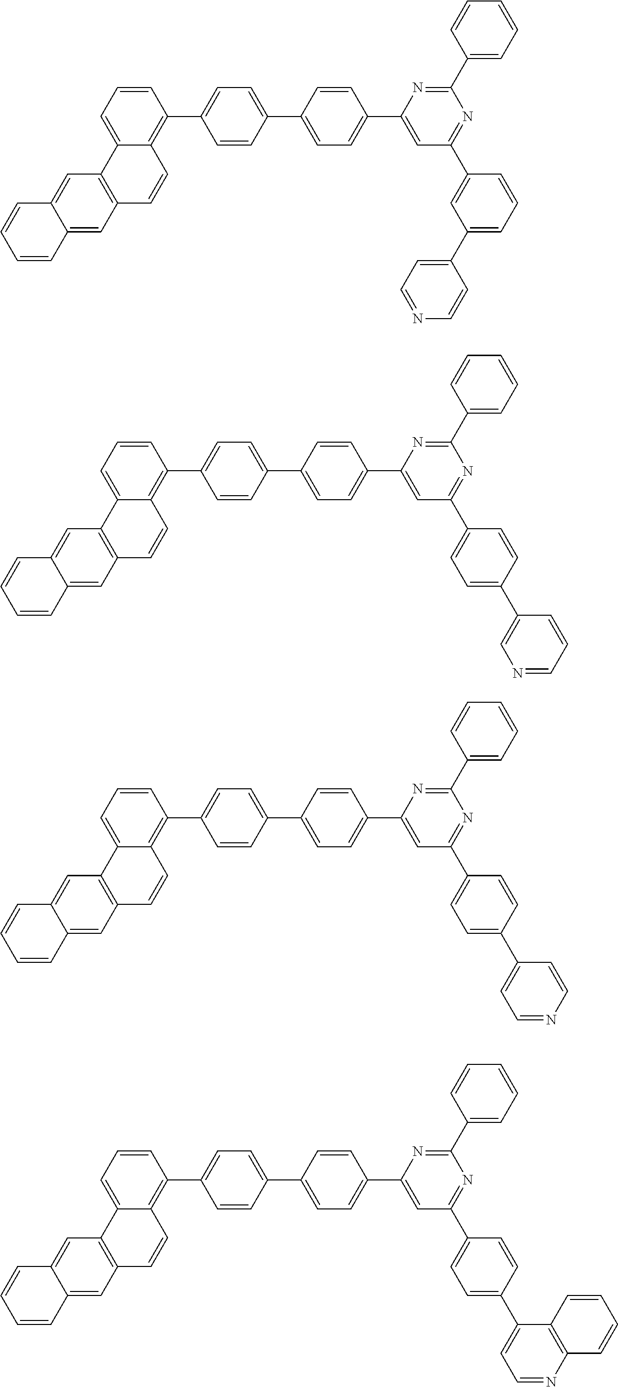

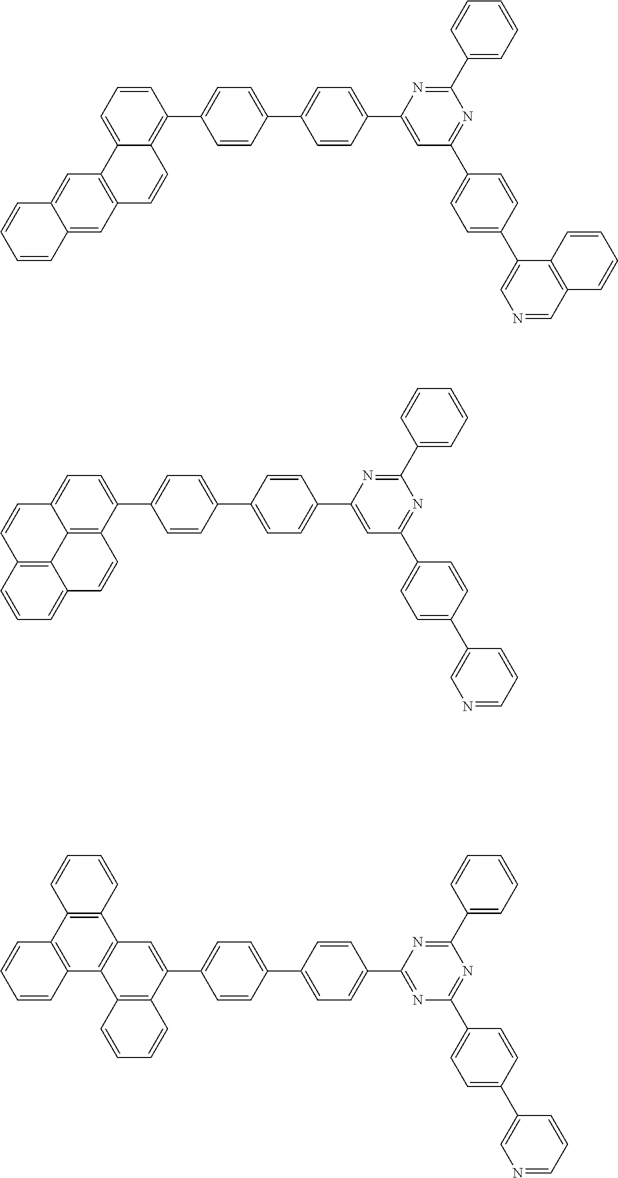 [SMILES: c1ccc(-c2nc(-c3ccc(-c4ccc(-c5cccc6c5ccc5cc7ccccc7cc56)cc4)cc3)cc(-c3ccc(-c4cncc5ccccc45)cc3)n2)cc1 ; c1ccc(-c2nc(-c3ccc(-c4ccc(-c5ccc6ccc7cccc8ccc5c6c78)cc4)cc3)cc(-c3ccc(-c4cccnc4)cc3)n2)cc1 ; c1ccc(-c2nc(-c3ccc(-c4ccc(-c5cc6c7ccccc7c7ccccc7c6c6ccccc56)cc4)cc3)nc(-c3ccc(-c4cccnc4)cc3)n2)cc1]

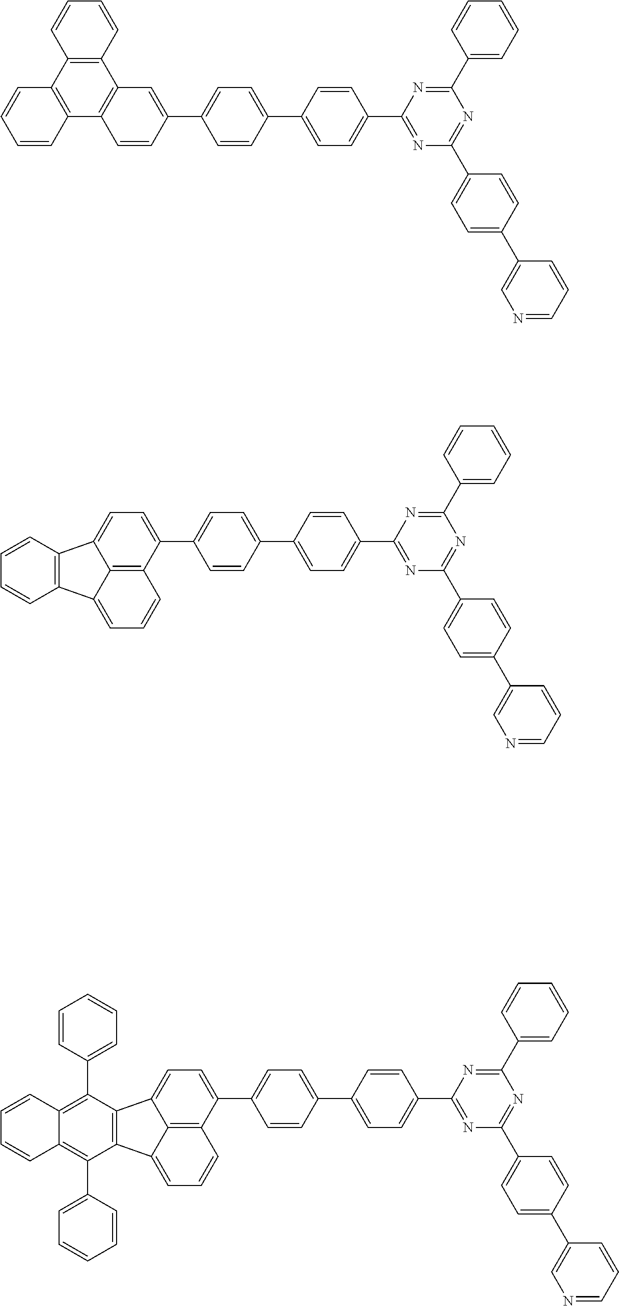 [SMILES: c1ccc(-c2nc(-c3ccc(-c4ccc(-c5ccc6c7ccccc7c7ccccc7c6c5)cc4)cc3)nc(-c3ccc(-c4cccnc4)cc3)n2)cc1 ; c1ccc(-c2nc(-c3ccc(-c4ccc(-c5ccc6c7c(cccc57)-c5ccccc5-6)cc4)cc3)nc(-c3ccc(-c4cccnc4)cc3)n2)cc1 ; c1ccc(-c2nc(-c3ccc(-c4ccc(-c5ccc6c7c(cccc57)-c5c-6c(-c6ccccc6)c6ccccc6c5-c5ccccc5)cc4)cc3)nc(-c3ccc(-c4cccnc4)cc3)n2)cc1]

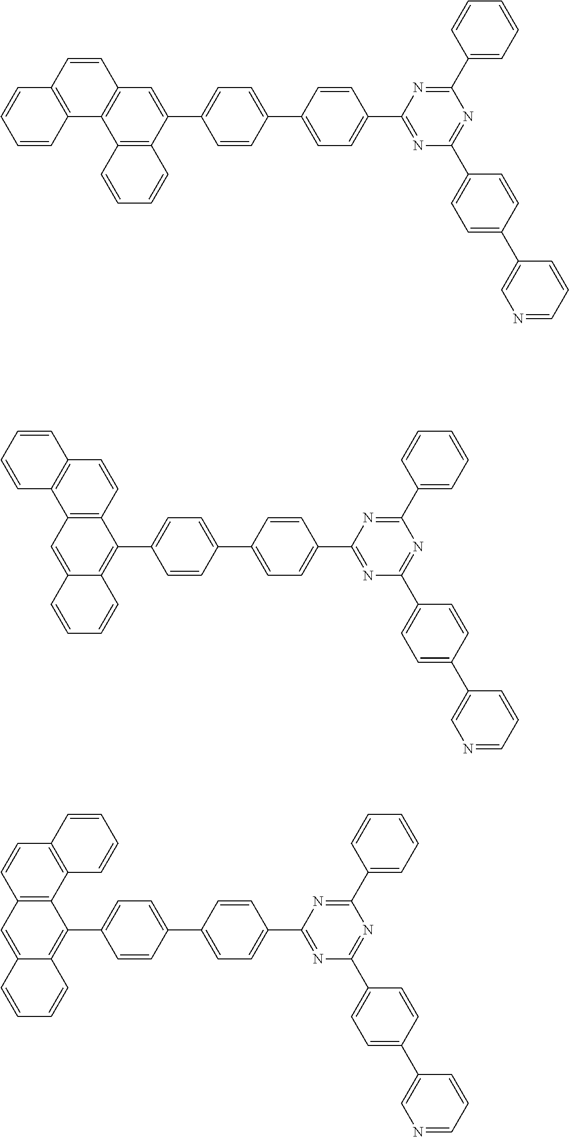 [SMILES: c1ccc(-c2nc(-c3ccc(-c4ccc(-c5cc6ccc7ccccc7c6c6ccccc56)cc4)cc3)nc(-c3ccc(-c4cccnc4)cc3)n2)cc1 ; c1ccc(-c2nc(-c3ccc(-c4ccc(-c5c6ccccc6cc6c5ccc5ccccc56)cc4)cc3)nc(-c3ccc(-c4cccnc4)cc3)n2)cc1 ; c1ccc(-c2nc(-c3ccc(-c4ccc(-c5c6ccccc6cc6ccc7ccccc7c56)cc4)cc3)nc(-c3ccc(-c4cccnc4)cc3)n2)cc1]

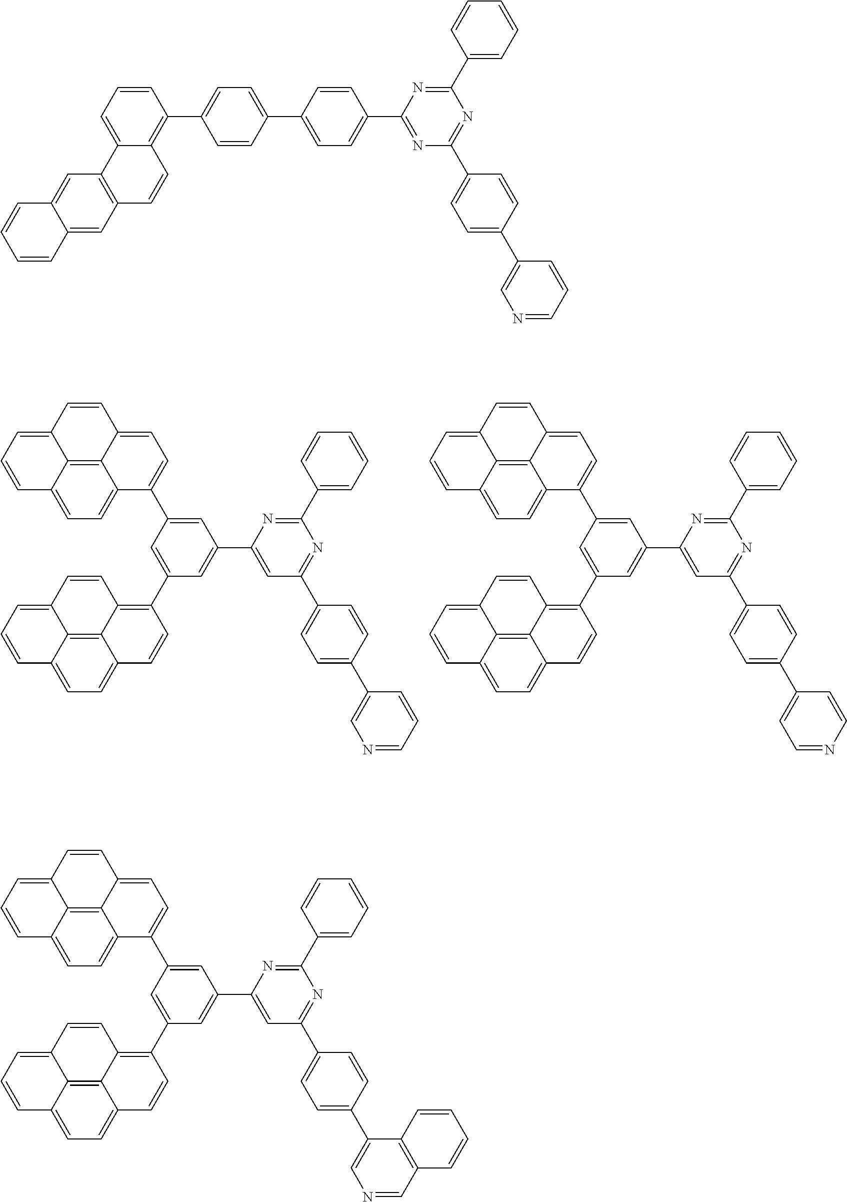 [SMILES: c1ccc(-c2nc(-c3ccc(-c4ccc(-c5cccc6c5ccc5cc7ccccc7cc56)cc4)cc3)nc(-c3ccc(-c4cccnc4)cc3)n2)cc1 ; c1ccc(-c2nc(-c3ccc(-c4cccnc4)cc3)cc(-c3cc(-c4ccc5ccc6cccc7ccc4c5c67)cc(-c4ccc5ccc6cccc7ccc4c5c67)c3)n2)cc1 ; c1ccc(-c2nc(-c3ccc(-c4ccncc4)cc3)cc(-c3cc(-c4ccc5ccc6cccc7ccc4c5c67)cc(-c4ccc5ccc6cccc7ccc4c5c67)c3)n2)cc1 ; c1ccc(-c2nc(-c3ccc(-c4cncc5ccccc45)cc3)cc(-c3cc(-c4ccc5ccc6cccc7ccc4c5c67)cc(-c4ccc5ccc6cccc7ccc4c5c67)c3)n2)cc1]

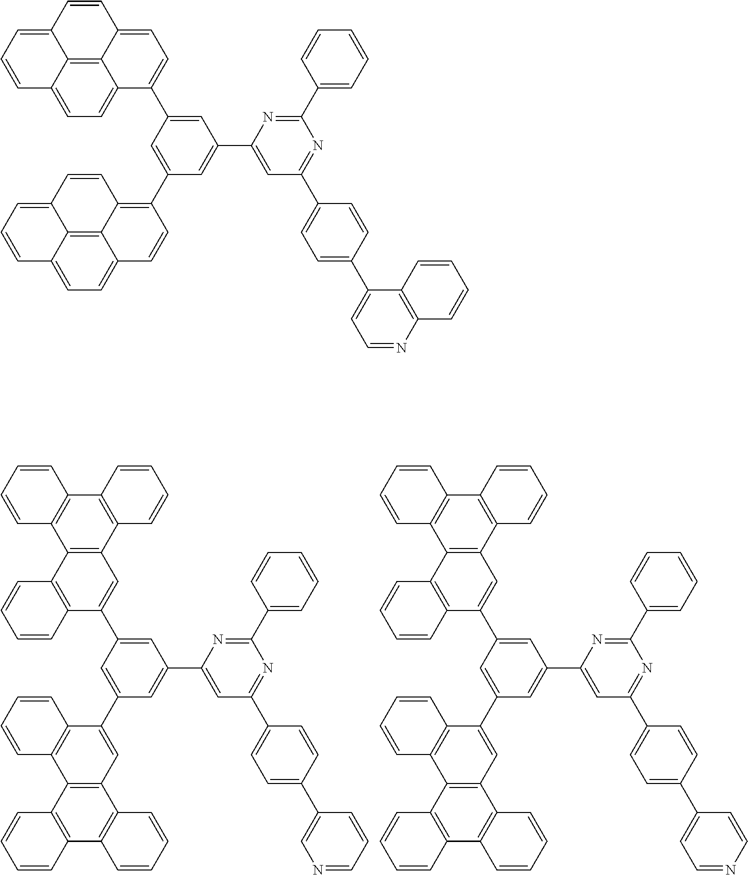

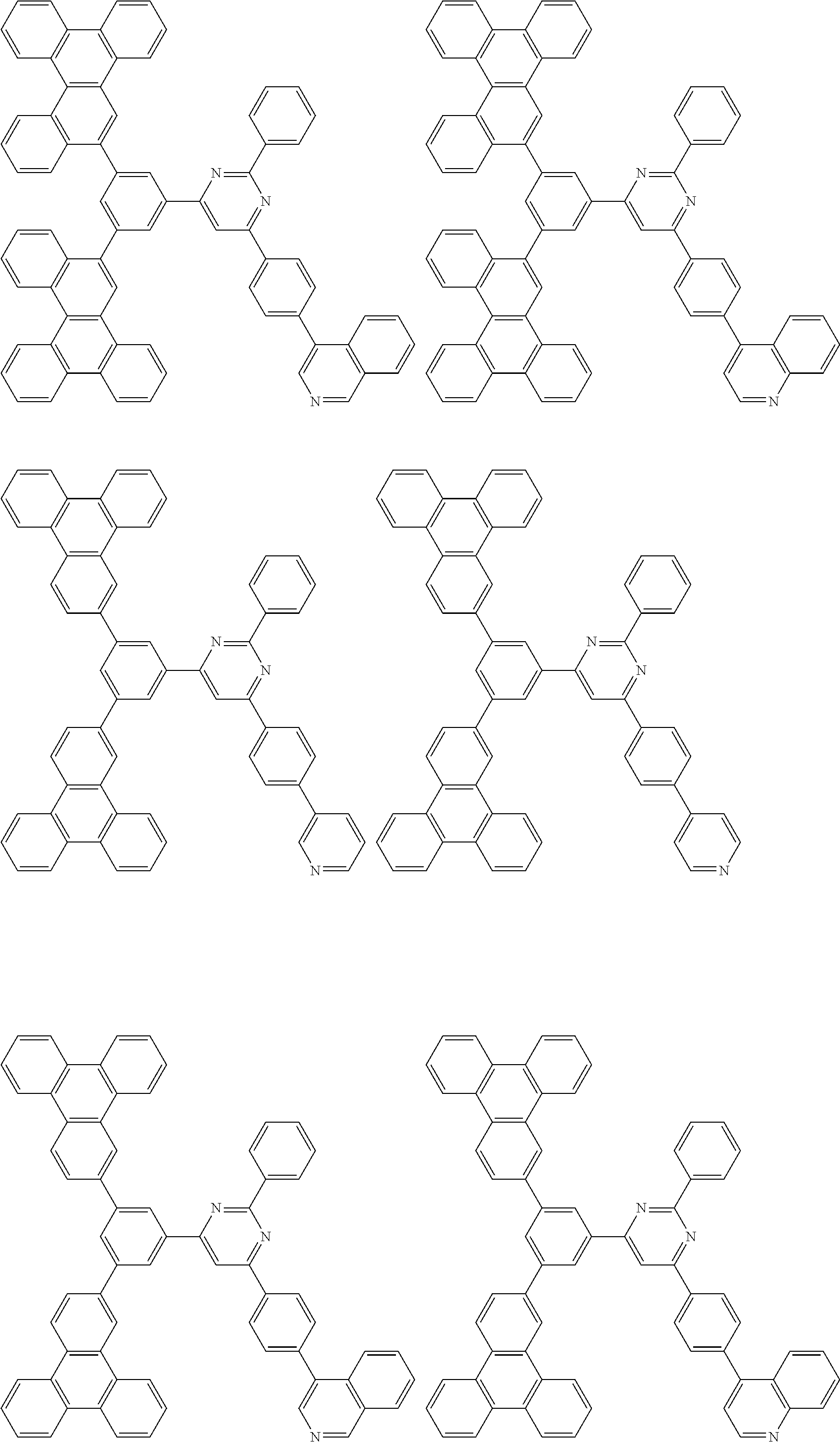

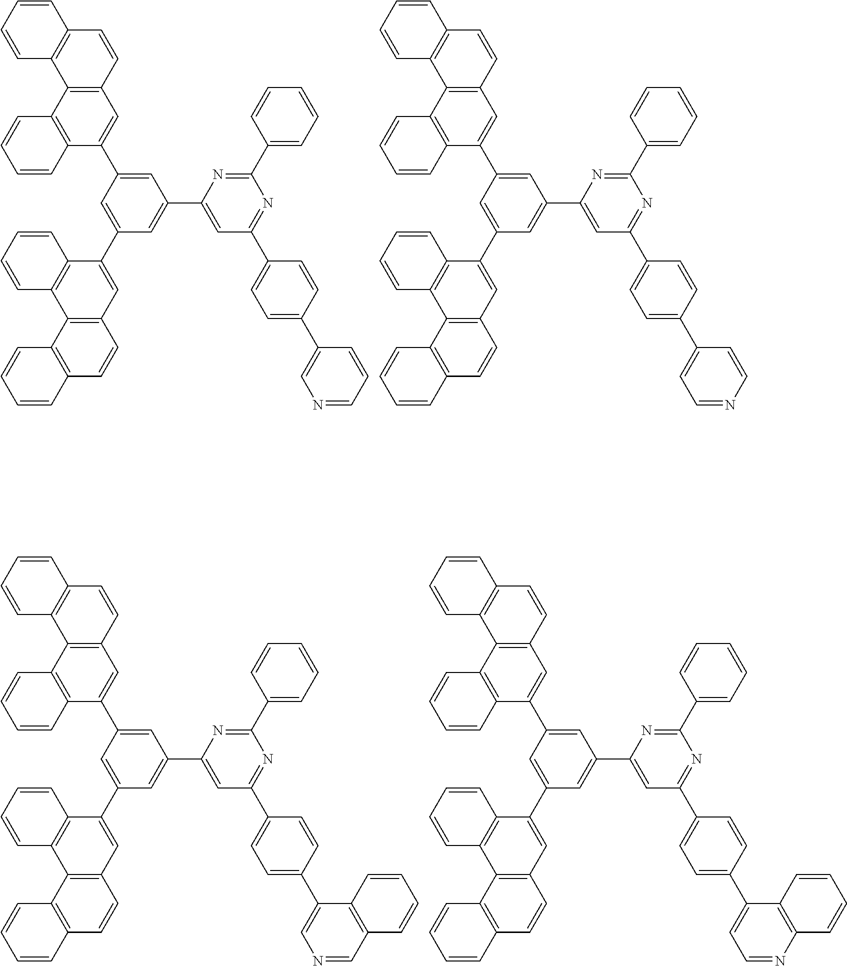

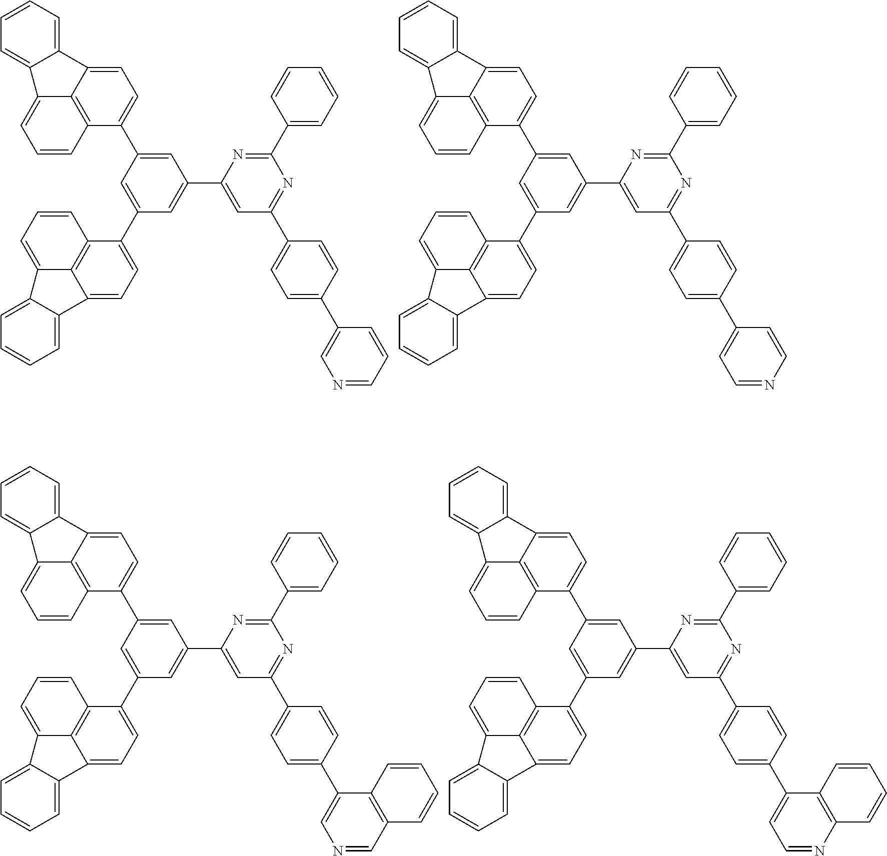

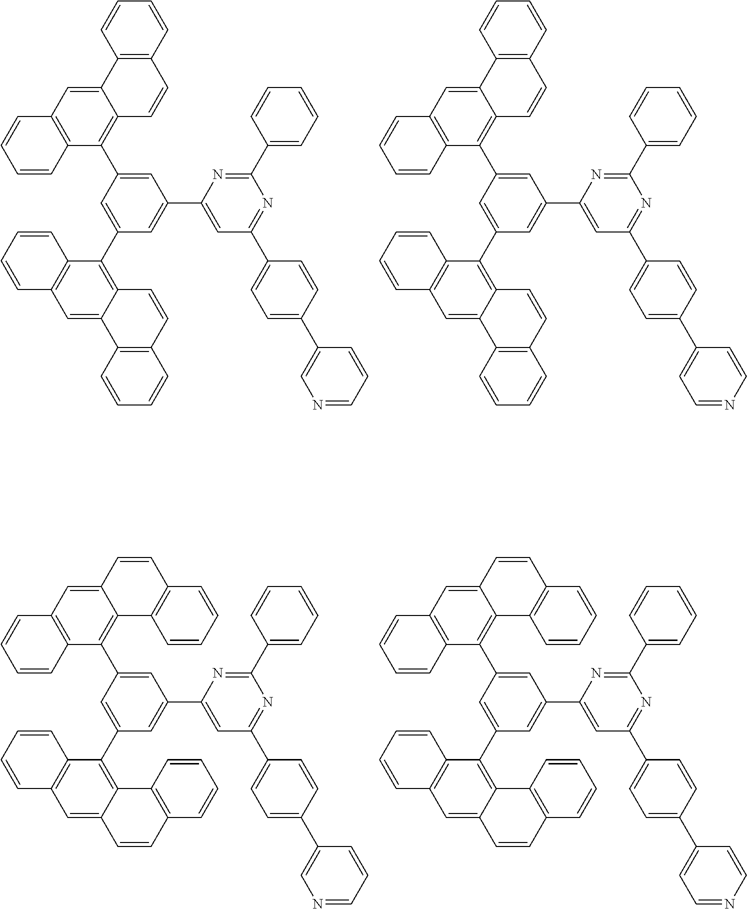 [SMILES: c1ccc(-c2nc(-c3ccc(-c4cccnc4)cc3)cc(-c3cc(-c4c5ccccc5cc5c4ccc4ccccc45)cc(-c4c5ccccc5cc5c4ccc4ccccc45)c3)n2)cc1 ; c1ccc(-c2nc(-c3ccc(-c4ccncc4)cc3)cc(-c3cc(-c4c5ccccc5cc5c4ccc4ccccc45)cc(-c4c5ccccc5cc5c4ccc4ccccc45)c3)n2)cc1 ; c1ccc(-c2nc(-c3ccc(-c4cccnc4)cc3)cc(-c3cc(-c4c5ccccc5cc5ccc6ccccc6c45)cc(-c4c5ccccc5cc5ccc6ccccc6c45)c3)n2)cc1 ; c1ccc(-c2nc(-c3ccc(-c4ccncc4)cc3)cc(-c3cc(-c4c5ccccc5cc5ccc6ccccc6c45)cc(-c4c5ccccc5cc5ccc6ccccc6c45)c3)n2)cc1]

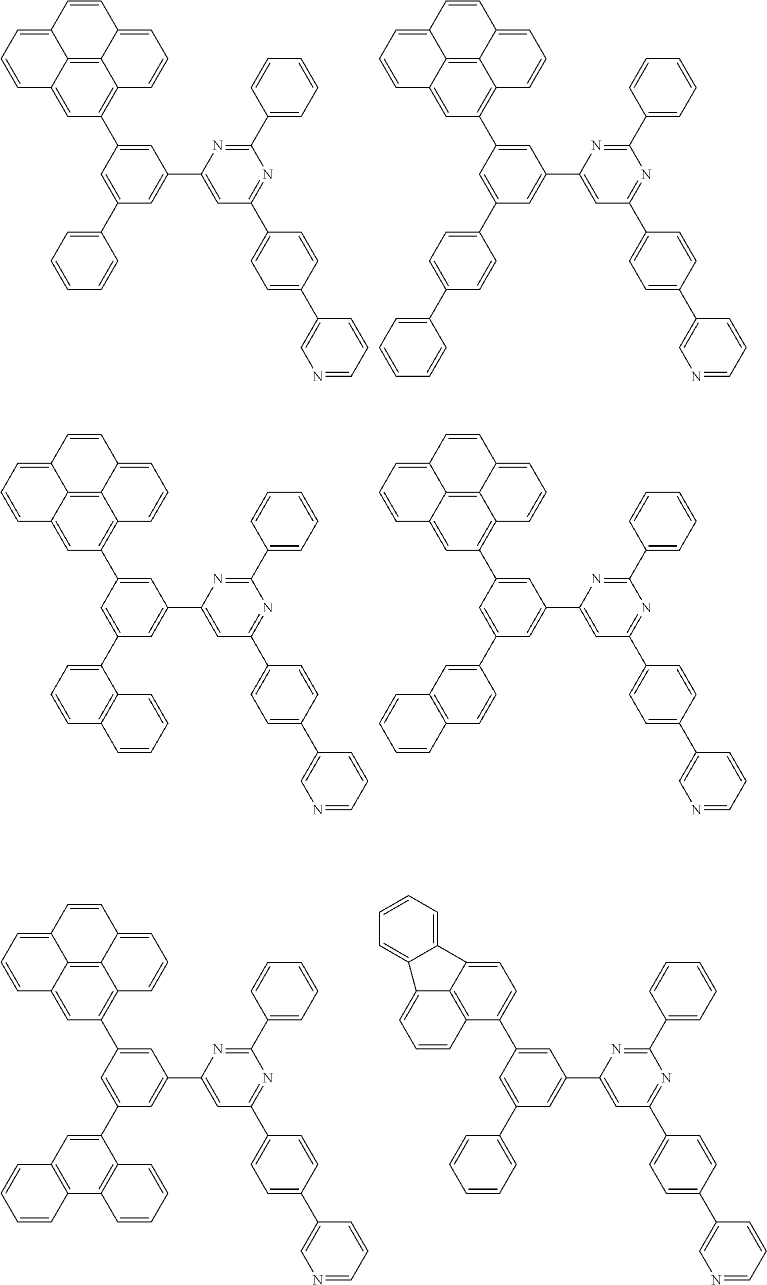

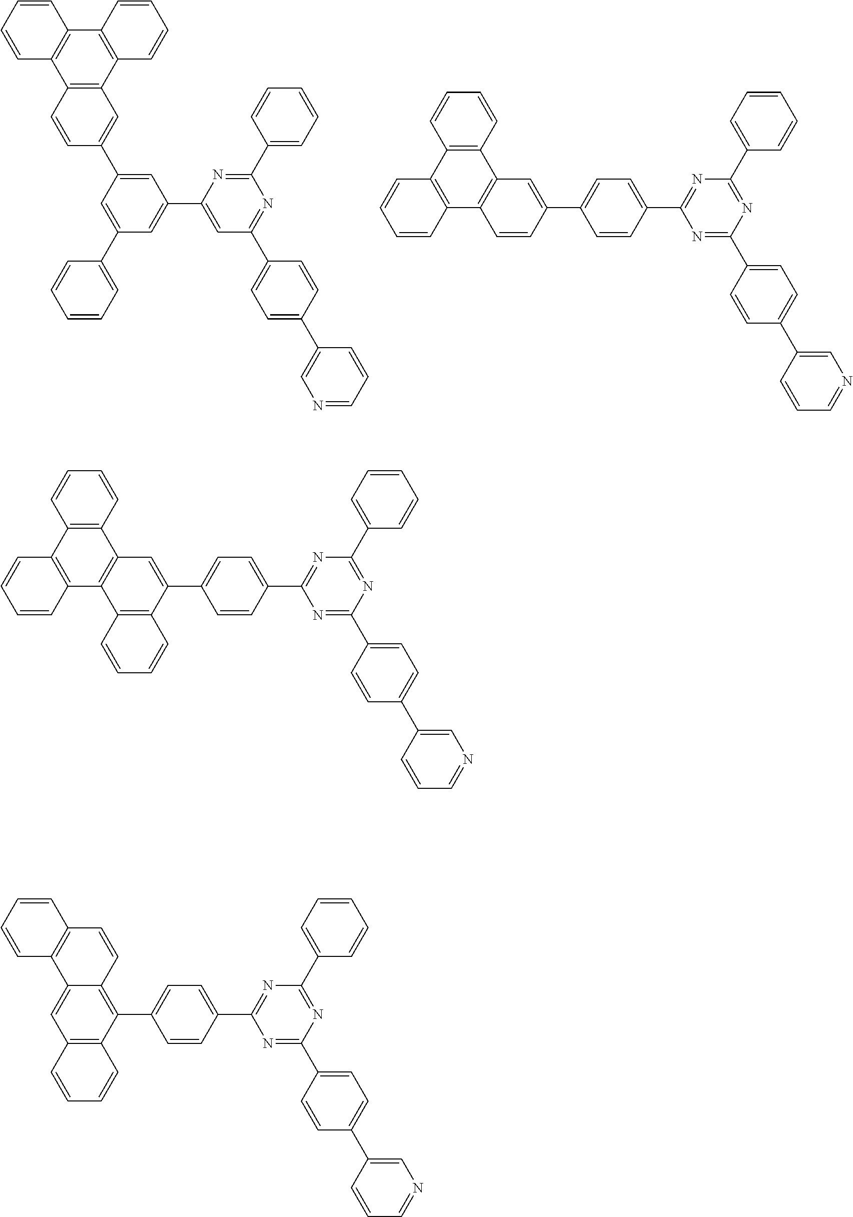 [SMILES: c1ccc(-c2cc(-c3ccc4c5ccccc5c5ccccc5c4c3)cc(-c3cc(-c4ccc(-c5cccnc5)cc4)nc(-c4ccccc4)n3)c2)cc1 ; c1ccc(-c2nc(-c3ccc(-c4cccnc4)cc3)nc(-c3ccc(-c4ccc5c6ccccc6c6ccccc6c5c4)cc3)n2)cc1 ; c1ccc(-c2nc(-c3ccc(-c4cccnc4)cc3)nc(-c3ccc(-c4cc5c6ccccc6c6ccccc6c5c5ccccc45)cc3)n2)cc1 ; c1ccc(-c2nc(-c3ccc(-c4cccnc4)cc3)nc(-c3ccc(-c4c5ccccc5cc5c4ccc4ccccc45)cc3)n2)cc1]

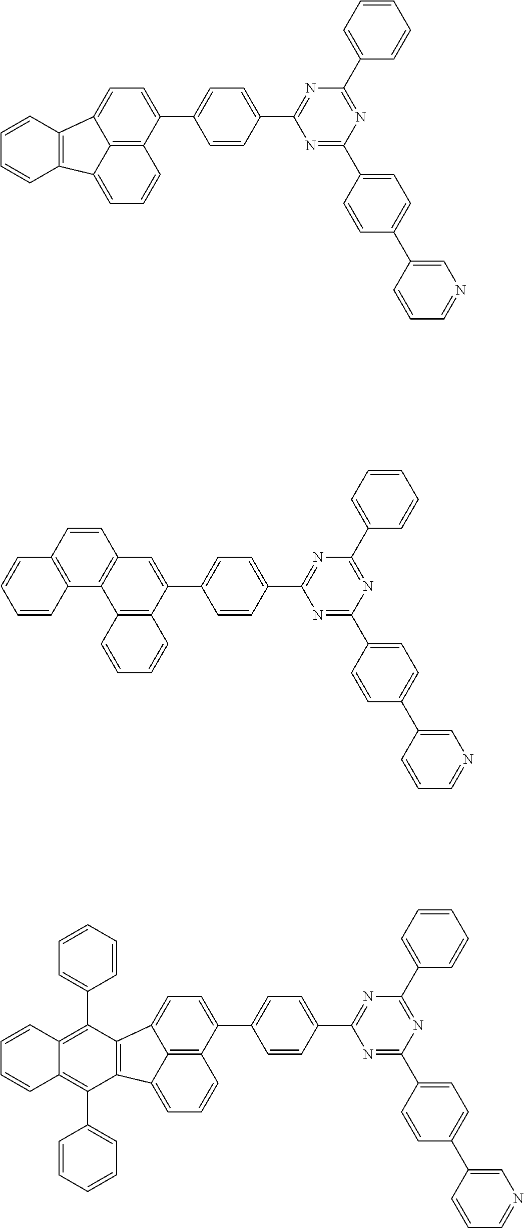 [SMILES: c1ccc(-c2nc(-c3ccc(-c4cccnc4)cc3)nc(-c3ccc(-c4ccc5c6c(cccc46)-c4ccccc4-5)cc3)n2)cc1 ; c1ccc(-c2nc(-c3ccc(-c4cccnc4)cc3)nc(-c3ccc(-c4cc5ccc6ccccc6c5c5ccccc45)cc3)n2)cc1 ; c1ccc(-c2nc(-c3ccc(-c4cccnc4)cc3)nc(-c3ccc(-c4ccc5c6c(cccc46)-c4c-5c(-c5ccccc5)c5ccccc5c4-c4ccccc4)cc3)n2)cc1]

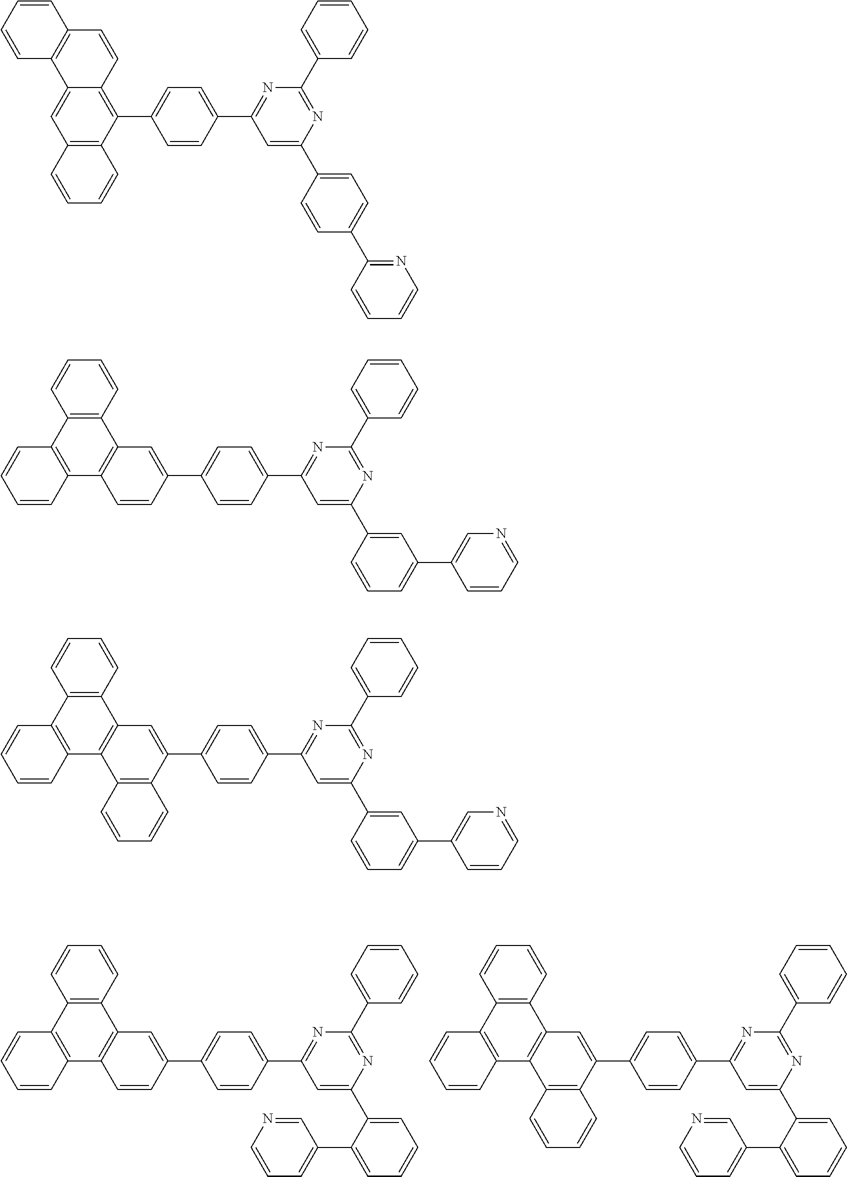

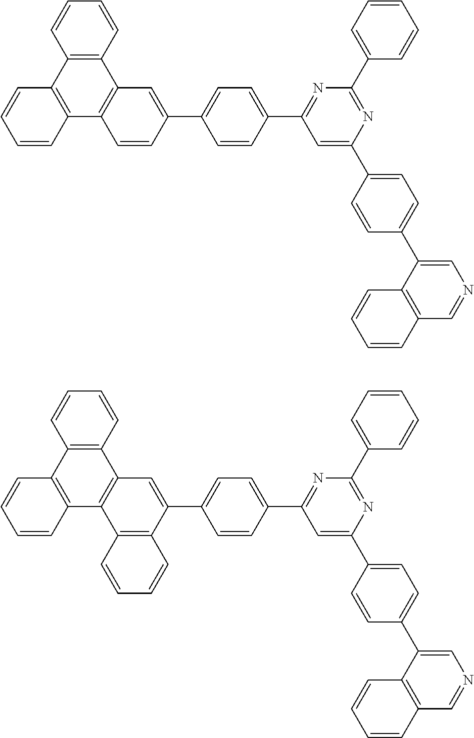

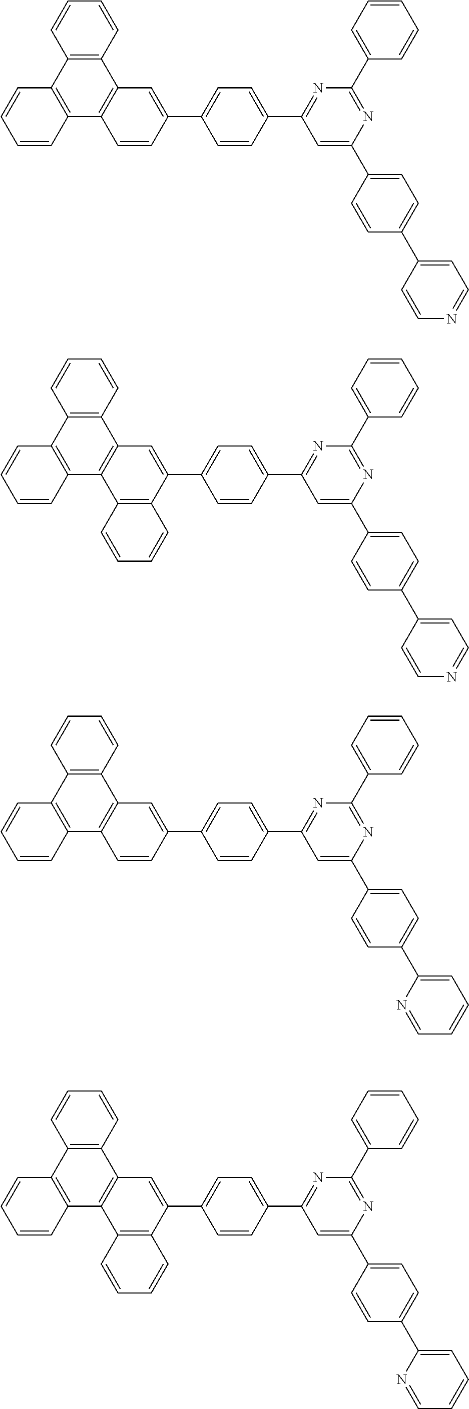

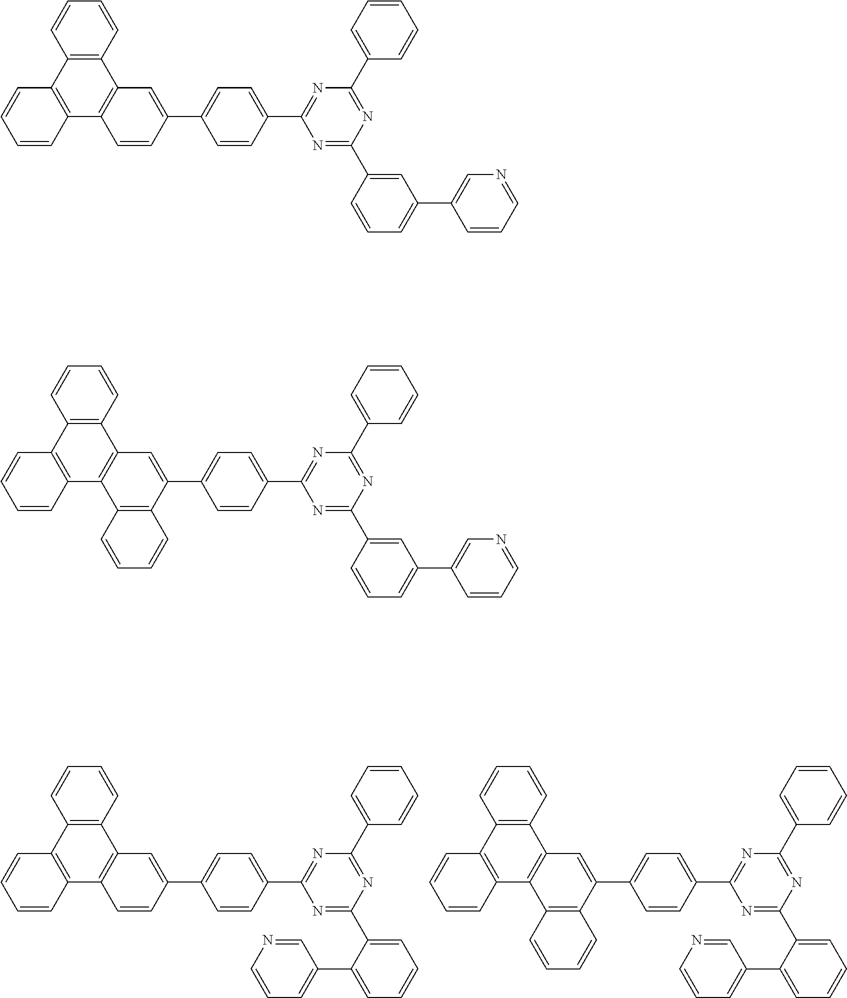 [SMILES: c1ccc(-c2nc(-c3ccc(-c4ccc5c6ccccc6c6ccccc6c5c4)cc3)nc(-c3cccc(-c4cccnc4)c3)n2)cc1 ; c1ccc(-c2nc(-c3ccc(-c4cc5c6ccccc6c6ccccc6c5c5ccccc45)cc3)nc(-c3cccc(-c4cccnc4)c3)n2)cc1 ; c1ccc(-c2nc(-c3ccc(-c4ccc5c6ccccc6c6ccccc6c5c4)cc3)nc(-c3ccccc3-c3cccnc3)n2)cc1 ; c1ccc(-c2nc(-c3ccc(-c4cc5c6ccccc6c6ccccc6c5c5ccccc45)cc3)nc(-c3ccccc3-c3cccnc3)n2)cc1]

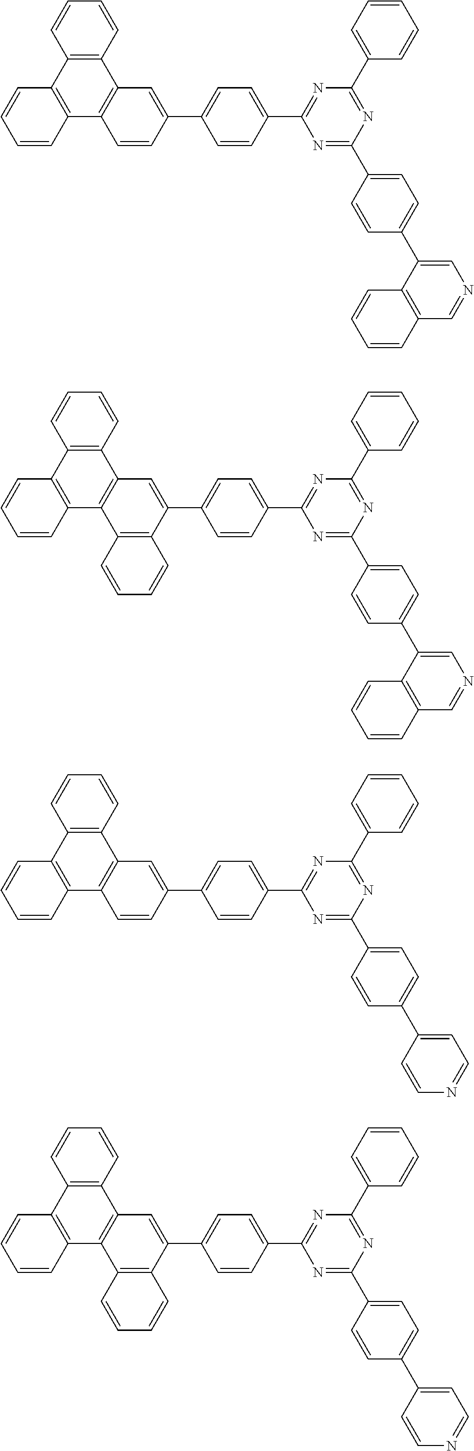

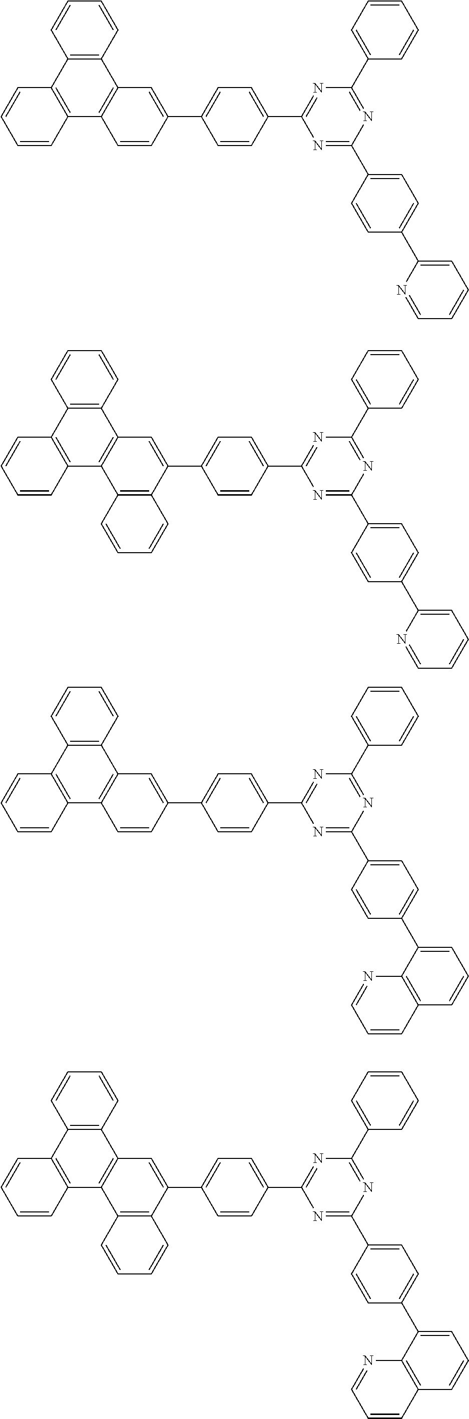

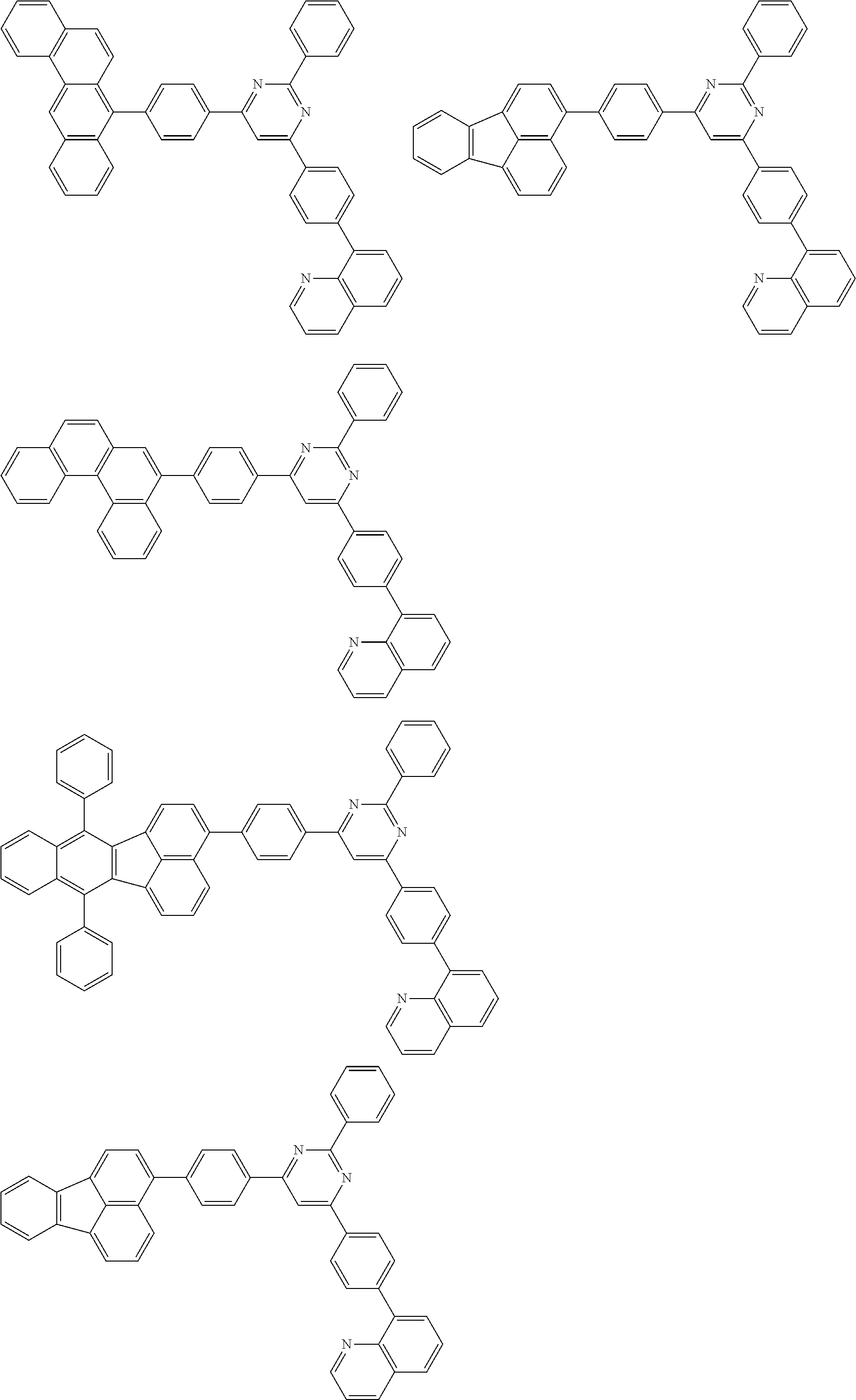

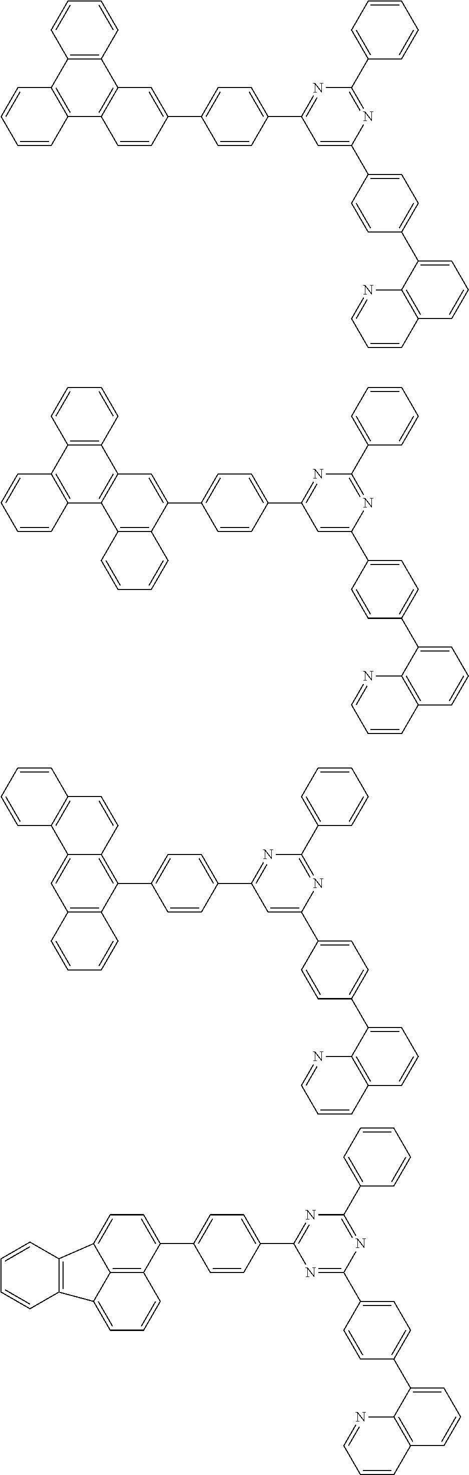

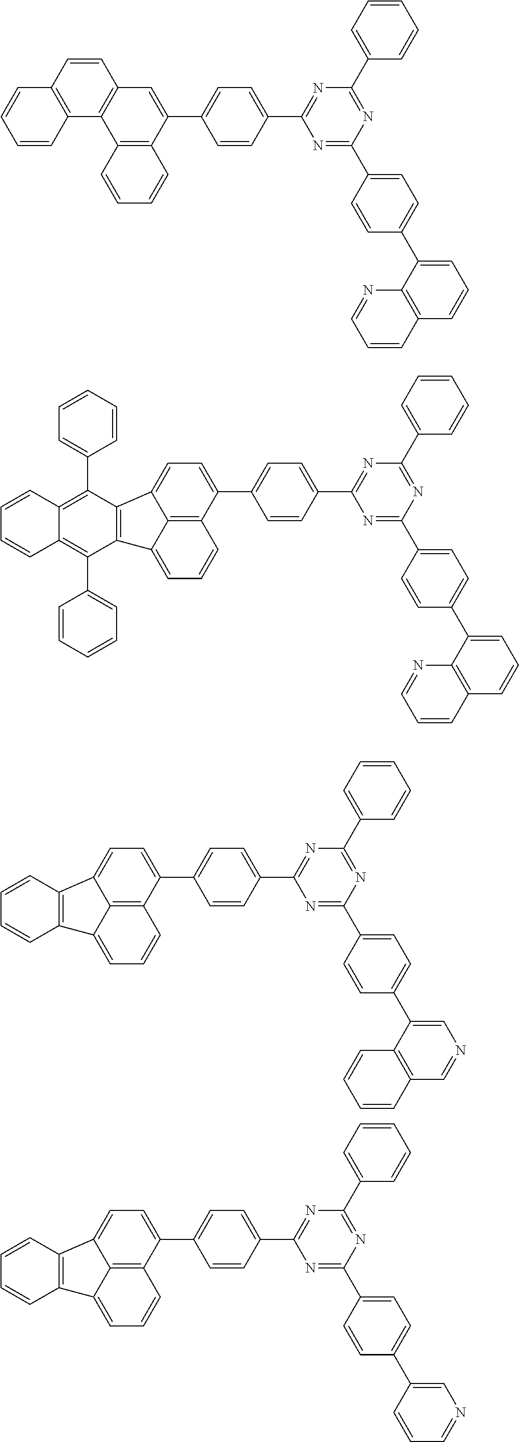

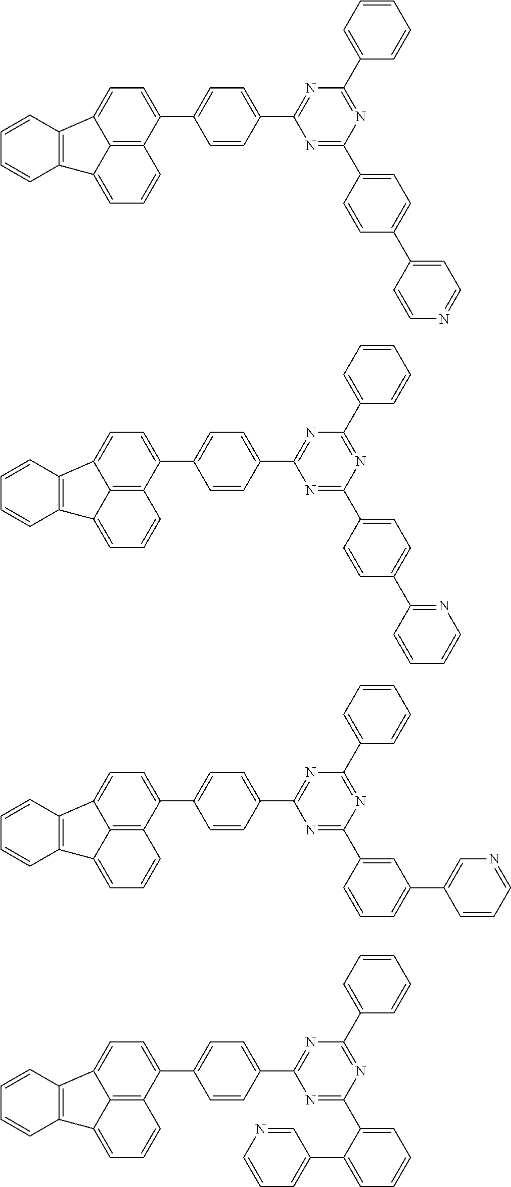

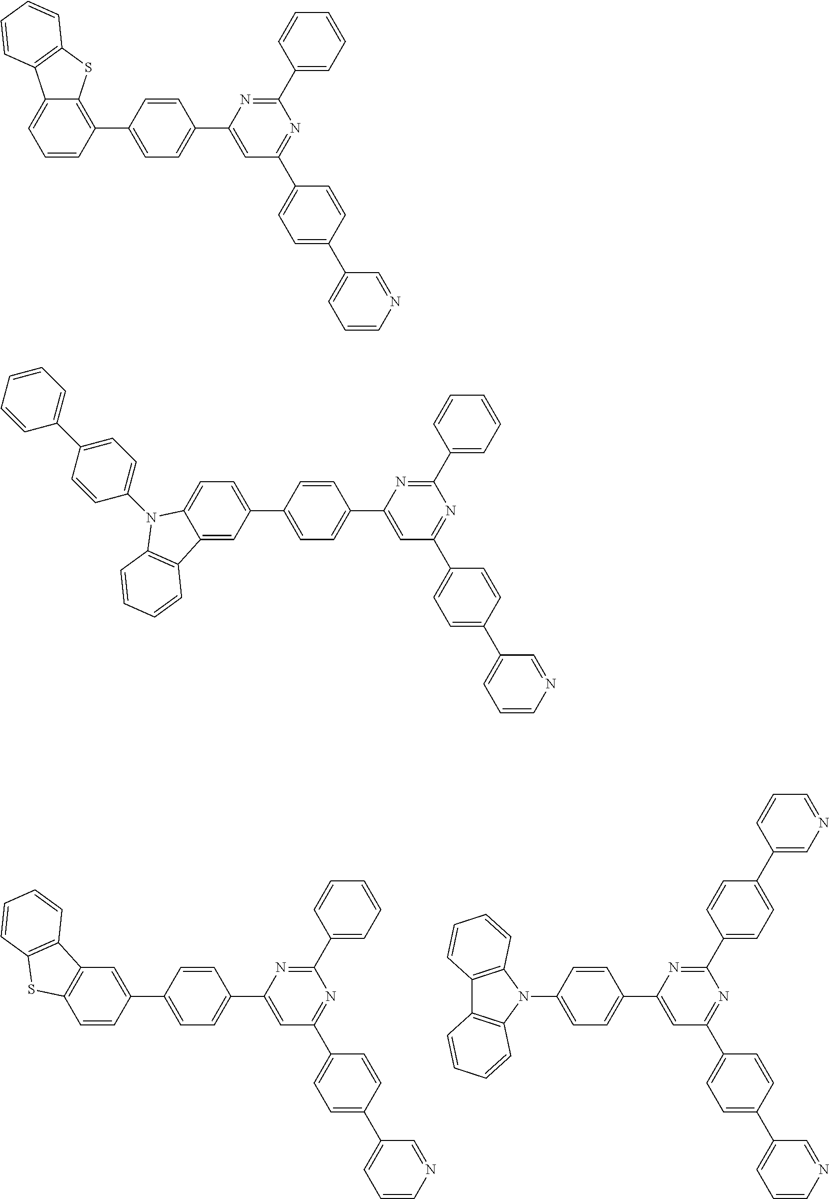 [SMILES: c1ccc(-c2nc(-c3ccc(-c4cccnc4)cc3)cc(-c3ccc(-c4cccc5c4sc4ccccc45)cc3)n2)cc1 ; c1ccc(-c2ccc(-n3c4ccccc4c4cc(-c5ccc(-c6cc(-c7ccc(-c8cccnc8)cc7)nc(-c7ccccc7)n6)cc5)ccc43)cc2)cc1 ; c1ccc(-c2nc(-c3ccc(-c4cccnc4)cc3)cc(-c3ccc(-c4ccc5sc6ccccc6c5c4)cc3)n2)cc1 ; c1cncc(-c2ccc(-c3cc(-c4ccc(-n5c6ccccc6c6ccccc65)cc4)nc(-c4ccc(-c5cccnc5)cc4)n3)cc2)c1]

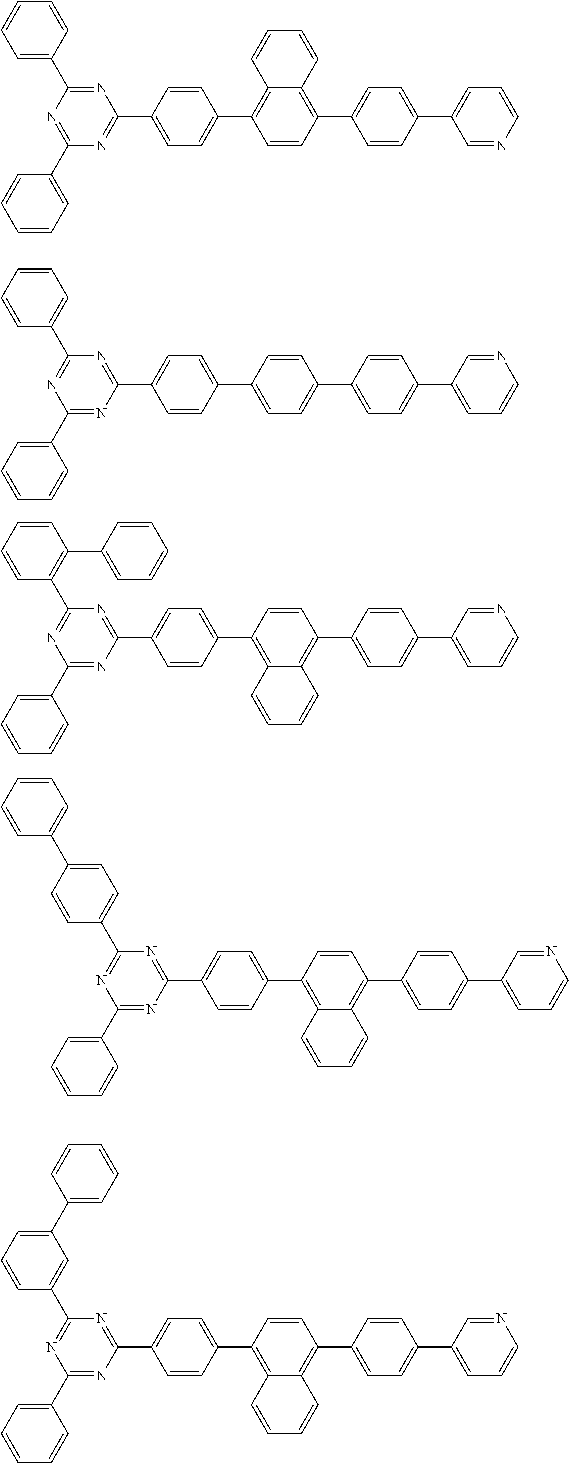

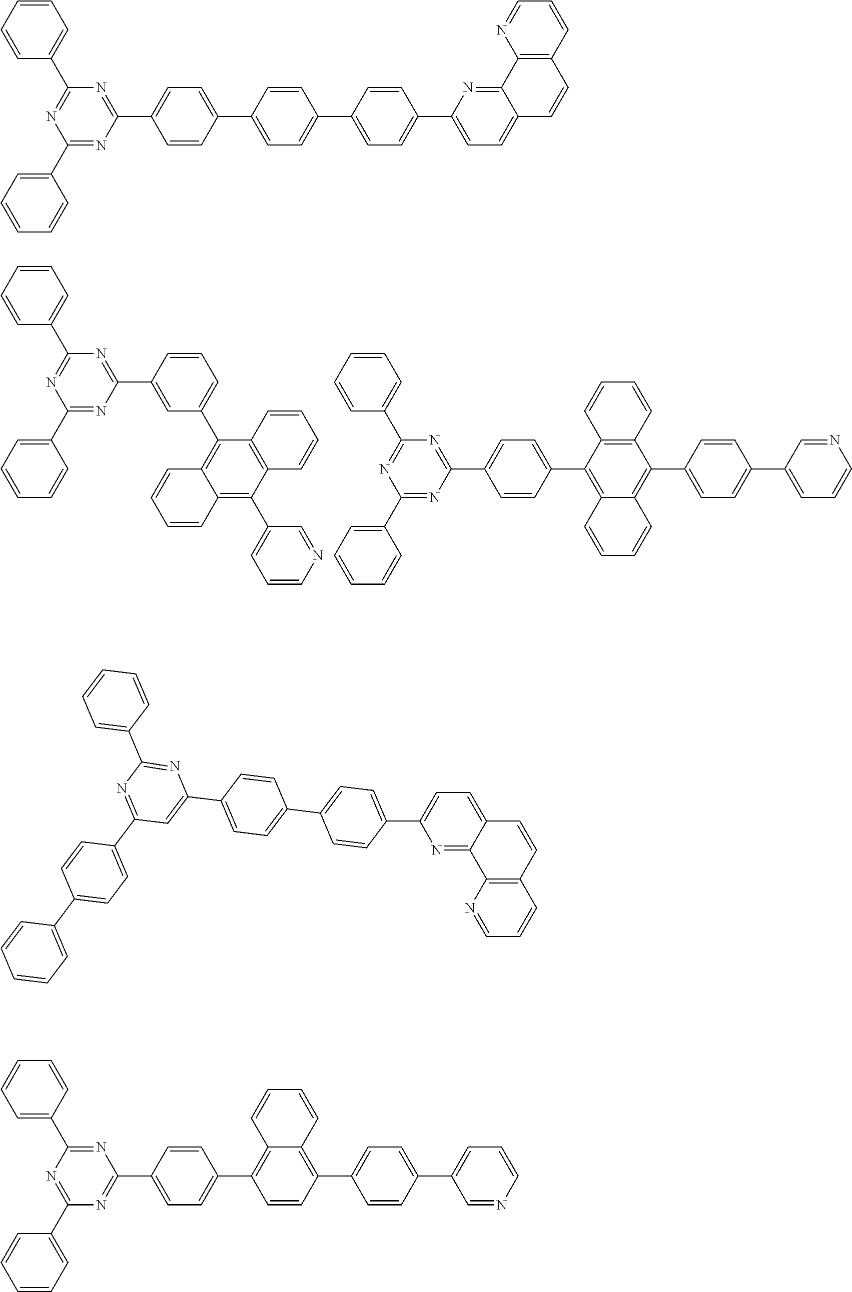

### [Compound Represented by Formula (4)]

A compound represented by the formula (4) is shown as follows:

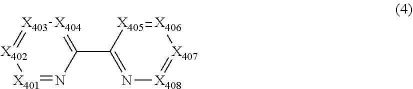

wherein in the formula (4),
X₄₀₁ to X₄₀ₛ are independently N, CR₄₀₁ or CR₄₀₂; provided that at least one of X₄₀₁ to X₄₀₈ is CR₄₀₁; when two or more R₄₀₁'s are present, the two or more R₄₀₁'s may be the same as or different from each other; when two or more R₄₀₂'s are present, the two or more R₄₀₂'s may be the same as or different from each other;
when both of X₄₀₄ and X₄₀₅ are CR₄₀₂, the two R₄₀₂'s form a substituted or unsubstituted, saturated or unsaturated fused ring by bonding with each other, or do not form the substituted or unsubstituted, saturated or unsaturated fused ring;
R₄₀₁ is a monovalent group represented by the following formula (4A):

(Ar₄₀₁)_{m4A}—(L₄₀₁)_{n4A}—* (4A)

wherein in the formula (4A),
L₄₀₁ is
a single bond,
a substituted or unsubstituted aromatic hydrocarbon ring group having 6 to 50 ring carbon atoms, or
a substituted or unsubstituted heterocyclic group having 5 to 50 ring atoms;
Ar₄₀₁ is
a hydrogen atom,
a substituted or unsubstituted aryl group having 6 to 50 ring carbon atoms, or
a substituted or unsubstituted monovalent heterocyclic group having 5 to 50 ring atoms; m4A is an integer of 1 to 5; when m4A is 2 or more, two or more Ar₄₁'s may be the same as or different from each other;
n4A is an integer of 1 to 3; when n4A is 2 or more, two or more L₄₀₁'s may be the same as or different from each other; provided that when all of L₄₀₁'s are a single bond, m4A is 1;
R₄₀₂ which do not form the substituted or unsubstituted, saturated or unsaturated fused ring is a hydrogen atom, or a substituent R; and
the substituent R is the same as defined in the formula (1).

When all of L₄₀₁'s is a single bond in the formula (4A), Ar₄₀₁ is directly bonded with the carbon atom of CR₄₀₁.

When m4A is 2 or more in the formula (4A), each of two or more Ar₄'s is directly bonded with L₄₀₁. Further, when a plurality of L₄₀₁'s is present (when n4A is 2 or more), Ar₄₀₁ is directly bonded with leftmost L₄₀₁ on paper. When m4A is 2 or more, each of two or more Ar₄'s is directly bonded with leftmost L₄₀₁ on paper. For example, when m4A is 2 and nA4 is 2, the group represented by the formula (4A) is the following structure.

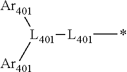

In one embodiment, the compound represented by the formula (4) is a compound represented by the following formula (4-1) or (4-2):

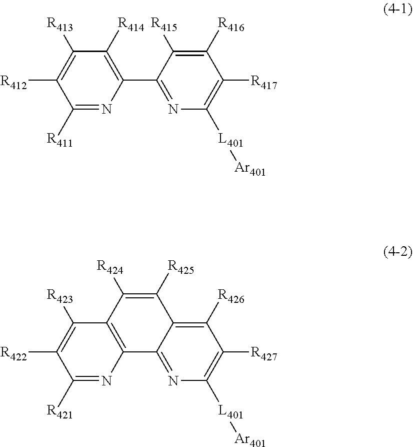

wherein in the formulas (4-1) and (4-2),
L₄₀₁, and Ar₄₀₁ are the same as defined in the formula (4);
R₄₁₁ to R₄₁₇, and R₄₂₁ to R₄₂₇ are independently a hydrogen atom, or a substituent R; and
the substituent R is the same as defined in the formula (1).

In one embodiment, the monovalent group represented by the formula (4A) is a monovalent group represented by the following formula (4A-1):

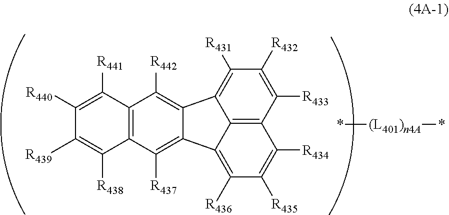

wherein in the formula (4A-1), L₄₀₁ and n4A are the same as defined in the formula (4A);
one of R₄₃₁ to R₄₄₂ is bonded with L₄₀₁ via a single bond;
R₄₃₁ to R₄₄₂ which are not bonded with L₄₀₁ are independently a hydrogen atom, or a substituent R; and
the substituent R is the same as defined in the formula (1).

In one embodiment, the compound represented by the formula (4) is a compound represented by the following formula (4-11) or (4-12):

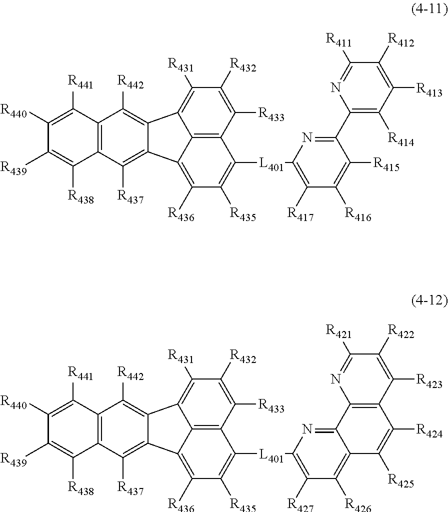

wherein in the formulas (4-11) and (4-12),
L₄₀₁ is the same as defined in the formula (4);
R₄₁₁ to R₄₁₇, R₄₂₁ to R₄₂₇, R₄₃₁ to R₄₃₃, and R₄₃₅ to R₄₄₂ are independently a hydrogen atom, or a substituent R; and
the substituent R is the same as defined in the formula (1).

In one embodiment, the monovalent group represented by the formula (4A) is a monovalent group represented by the following formula (4A-2):

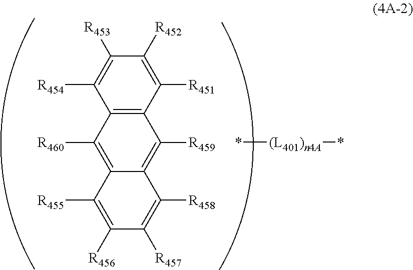

wherein in the formula (4A-2), L₄₀₁ and n4A are the same as defined in the formula (4A);
one of R₄₅₁ to R₄₆₀ is bonded with L₄₀₁ via a single bond;
R₄₅₁ to R₄₆₀ which are not bonded with L₄₀₁ are independently a hydrogen atom, or a substituent R; and
the substituent R is the same as defined in the formula (1).

In one embodiment, the compound represented by the formula (4) is a compound represented by the following formula (4-21) or (4-22):

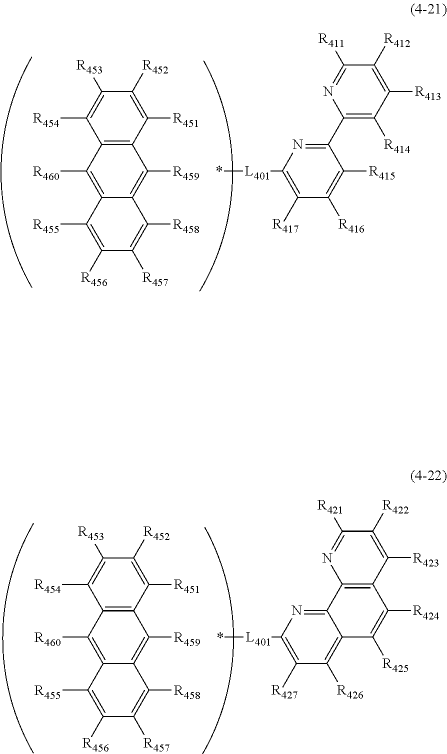

wherein in the formulas (4-21) and (4-22),
L₄₀₁ is the same as defined in the formula (4);
one of R₄₅₁ to R₄₆₀ is bonded with L₄₀₁ via a single bond;
R₄₁₁ to R₄₁₇, R₄₂₁ to R₄₂₇, and R₄₅₁ to R₄₆₀ which are not bonded with L₄₀₁ are independently
a hydrogen atom, or a substituent R; and
the substituent R is the same as defined in the formula (1).

In one embodiment, the compound represented by the formula (4-22) is a compound represented by the following formula (4-22-1) or (4-22-2):

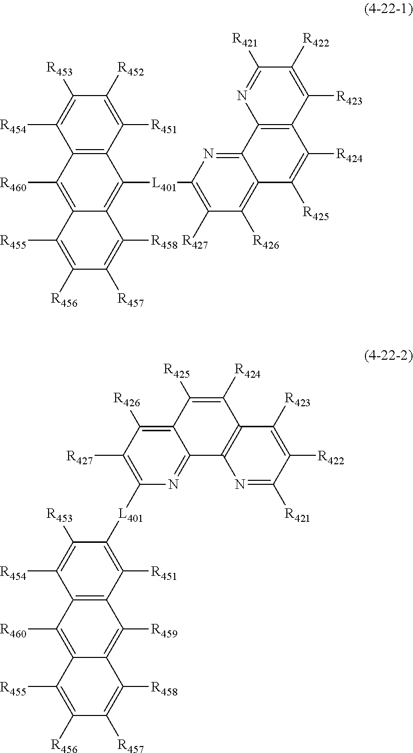

wherein in the formulas (4-22-1) and (4-22-2),
L₄₀₁, R₄₂₁ to R₄₂₇, and R₄₅₁ to R₄₆₀ are the same as defined in the formula (4-22).

In one embodiment, a substituent in the case of “substituted or unsubstituted” in the formula (4) is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,
—Si(R₄₁)(R₄₂)(R₄₃),
—C(═O)R₄₄, —COOR₄₅,
—S(═O)₂R₄₆,
—P(═O)(R₄₇)(R₄₈),
—Ge(R₄₉)(R₅₀)(R₅₁),
—N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other),
a hydroxy group,
a halogen atom,
a cyano group,
a nitro group,
an aryl group having 6 to 50 ring carbon atoms, and
a monovalent heterocyclic group having 5 to 50 ring atoms.

In one embodiment, a substituent in the case of “substituted or unsubstituted” in the formula (4) is selected from the group consisting of
an alkyl group having 1 to 18 carbon atoms,
an aryl group having 6 to 18 ring carbon atoms, and
a monovalent heterocyclic group having 5 to 18 ring atoms.

The compound represented by the formula (4) can be synthesized by using known alternative reactions or raw materials adapted to the target compound.

Specific examples of the compound represented by the formula (4) will be described below, but these are merely examples, and the compound represented by the formula (4) is not limited to the following specific examples.

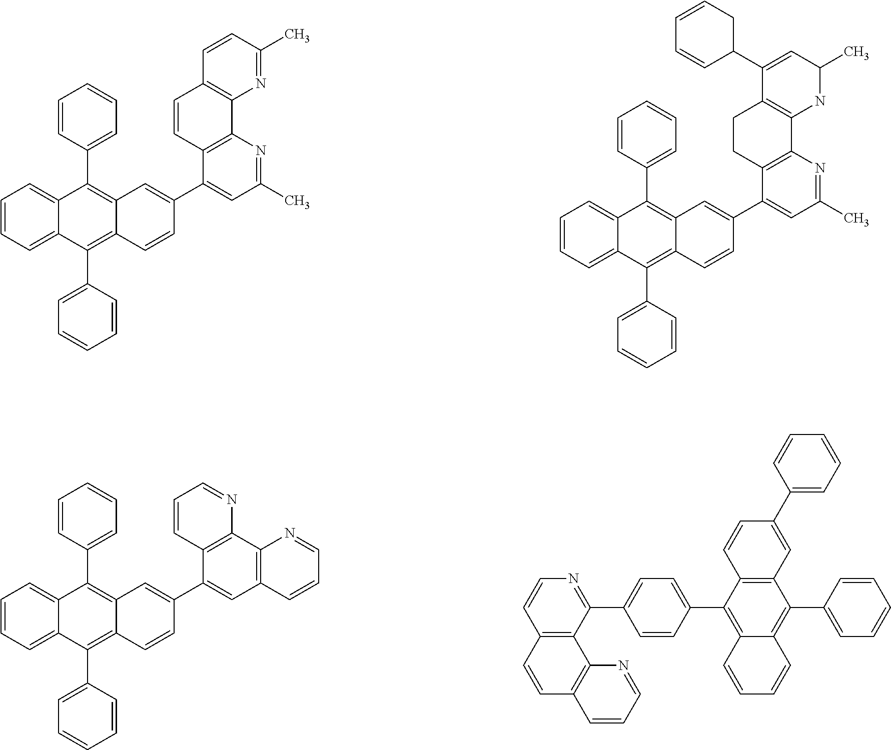 [SMILES: Cc1ccc2ccc3c(-c4ccc5c(-c6ccccc6)c6ccccc6c(-c6ccccc6)c5c4)cc(C)nc3c2n1 ; Cc1cc(-c2ccc3c(-c4ccccc4)c4ccccc4c(-c4ccccc4)c3c2)c2c(n1)C1=C(CC2)C(C2C=CC=CC2)=CC(C)N1 ; c1ccc(-c2c3ccccc3c(-c3ccccc3)c3cc(-c4cc5cccnc5c5ncccc45)ccc23)cc1 ; c1ccc(-c2ccc3c(-c4ccc(-c5nccc6ccc7cccnc7c56)cc4)c4ccccc4c(-c4ccccc4)c3c2)cc1]

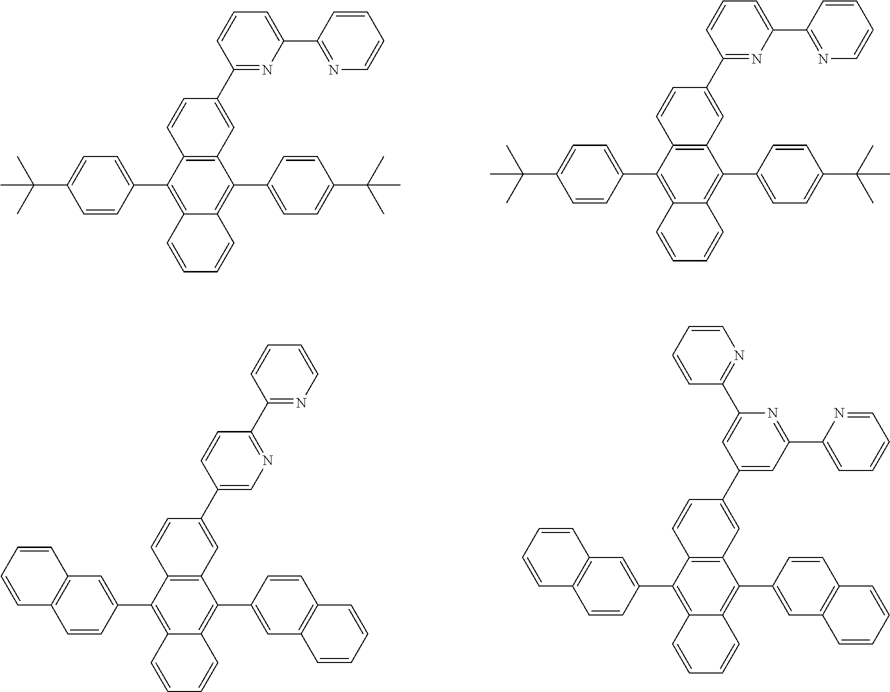 [SMILES: CC(C)(C)c1ccc(-c2c3ccccc3c(-c3ccc(C(C)(C)C)cc3)c3cc(-c4cccc(-c5ccccn5)n4)ccc23)cc1 ; CC(C)(C)c1ccc(-c2c3ccccc3c(-c3ccc(C(C)(C)C)cc3)c3cc(-c4cccc(-c5ccccn5)n4)ccc23)cc1 ; c1ccc(-c2ccc(-c3ccc4c(-c5ccc6ccccc6c5)c5ccccc5c(-c5ccc6ccccc6c5)c4c3)cn2)nc1 ; c1ccc(-c2cc(-c3ccc4c(-c5ccc6ccccc6c5)c5ccccc5c(-c5ccc6ccccc6c5)c4c3)cc(-c3ccccn3)n2)nc1]

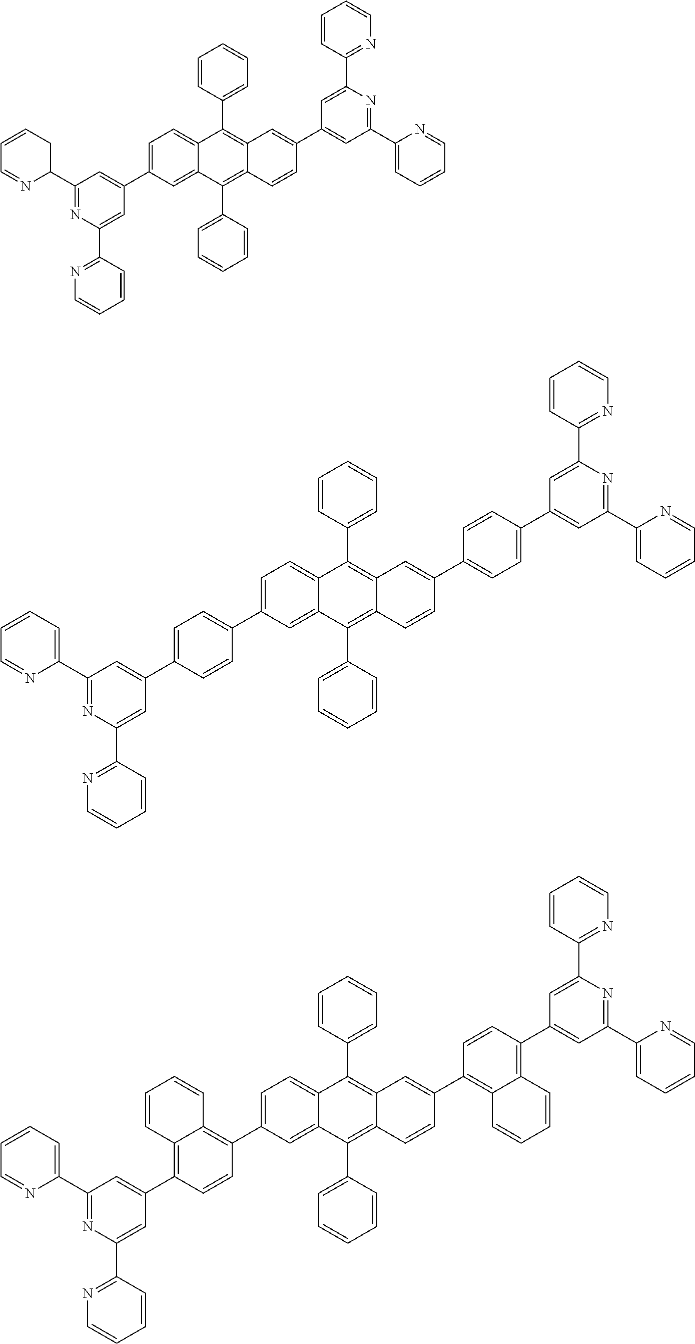 [SMILES: C1=CCC(c2cc(-c3ccc4c(-c5ccccc5)c5cc(-c6cc(-c7ccccn7)nc(-c7ccccn7)c6)ccc5c(-c5ccccc5)c4c3)cc(-c3ccccn3)n2)N=C1 ; c1ccc(-c2c3ccc(-c4ccc(-c5cc(-c6ccccn6)nc(-c6ccccn6)c5)cc4)cc3c(-c3ccccc3)c3ccc(-c4ccc(-c5cc(-c6ccccn6)nc(-c6ccccn6)c5)cc4)cc23)cc1 ; c1ccc(-c2c3ccc(-c4ccc(-c5cc(-c6ccccn6)nc(-c6ccccn6)c5)c5ccccc45)cc3c(-c3ccccc3)c3ccc(-c4ccc(-c5cc(-c6ccccn6)nc(-c6ccccn6)c5)c5ccccc45)cc23)cc1]

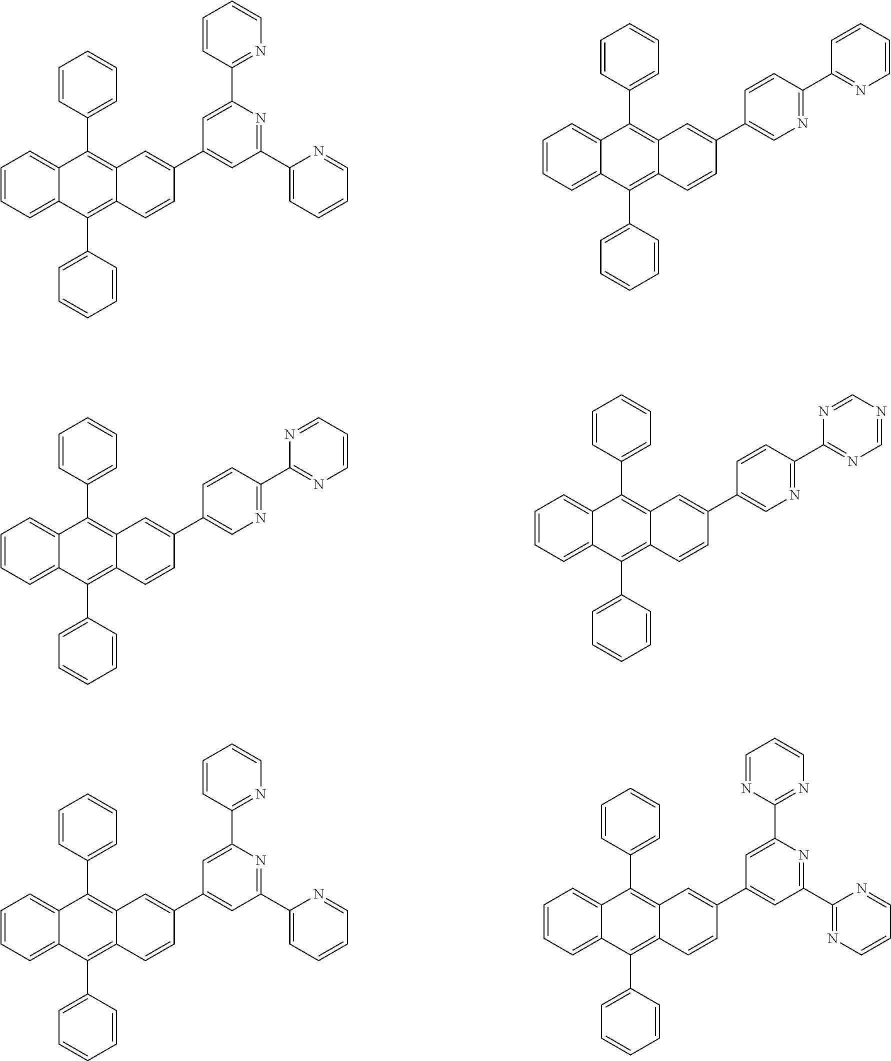 [SMILES: c1ccc(-c2c3ccccc3c(-c3ccccc3)c3cc(-c4cc(-c5ccccn5)nc(-c5ccccn5)c4)ccc23)cc1 ; c1ccc(-c2c3ccccc3c(-c3ccccc3)c3cc(-c4ccc(-c5ccccn5)nc4)ccc23)cc1 ; c1ccc(-c2c3ccccc3c(-c3ccccc3)c3cc(-c4ccc(-c5ncccn5)nc4)ccc23)cc1 ; c1ccc(-c2c3ccccc3c(-c3ccccc3)c3cc(-c4ccc(-c5ncncn5)nc4)ccc23)cc1 ; c1ccc(-c2c3ccccc3c(-c3ccccc3)c3cc(-c4cc(-c5ccccn5)nc(-c5ccccn5)c4)ccc23)cc1 ; c1ccc(-c2c3ccccc3c(-c3ccccc3)c3cc(-c4cc(-c5ncccn5)nc(-c5ncccn5)c4)ccc23)cc1]

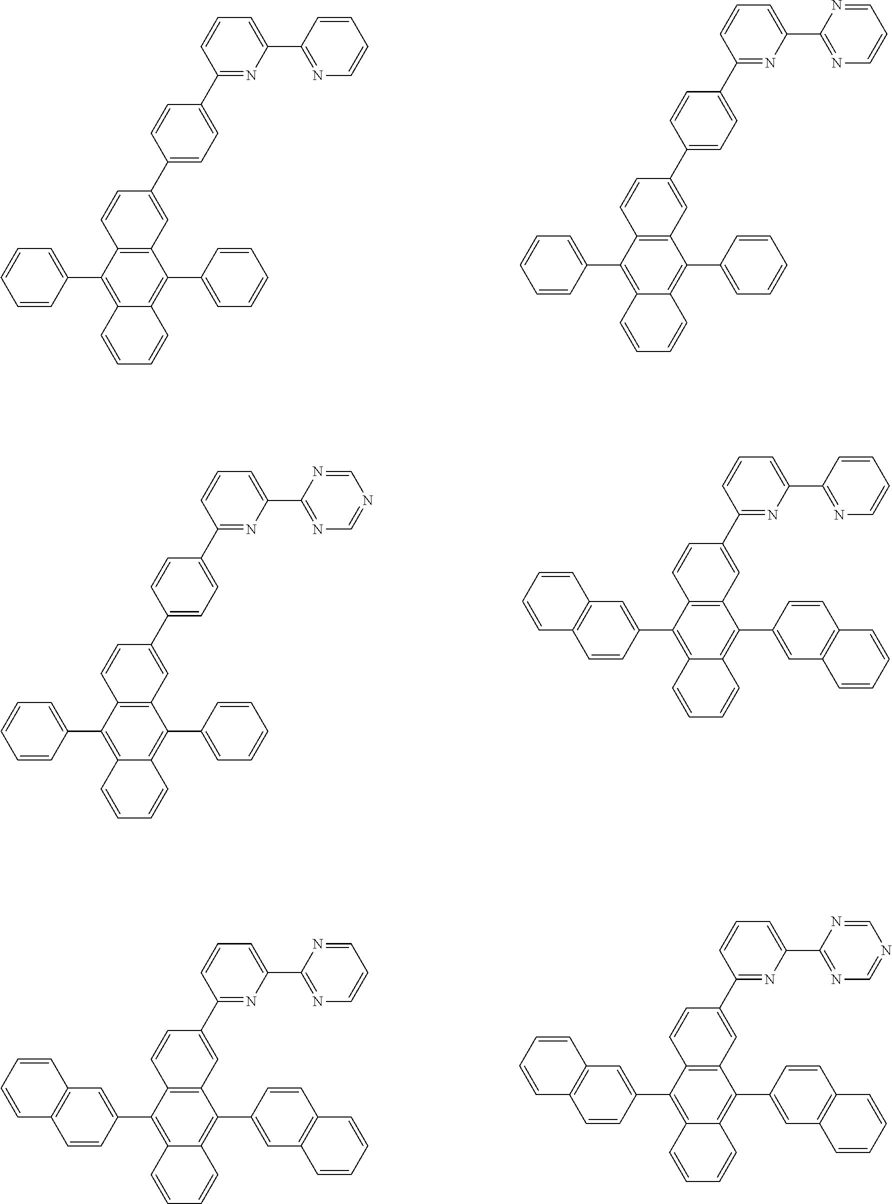 [SMILES: c1ccc(-c2c3ccccc3c(-c3ccccc3)c3cc(-c4ccc(-c5cccc(-c6ccccn6)n5)cc4)ccc23)cc1 ; c1ccc(-c2c3ccccc3c(-c3ccccc3)c3cc(-c4ccc(-c5cccc(-c6ncccn6)n5)cc4)ccc23)cc1 ; c1ccc(-c2c3ccccc3c(-c3ccccc3)c3cc(-c4ccc(-c5cccc(-c6ncncn6)n5)cc4)ccc23)cc1 ; c1ccc(-c2cccc(-c3ccc4c(-c5ccc6ccccc6c5)c5ccccc5c(-c5ccc6ccccc6c5)c4c3)n2)nc1 ; c1cnc(-c2cccc(-c3ccc4c(-c5ccc6ccccc6c5)c5ccccc5c(-c5ccc6ccccc6c5)c4c3)n2)nc1 ; c1cc(-c2ccc3c(-c4ccc5ccccc5c4)c4ccccc4c(-c4ccc5ccccc5c4)c3c2)nc(-c2ncncn2)c1]

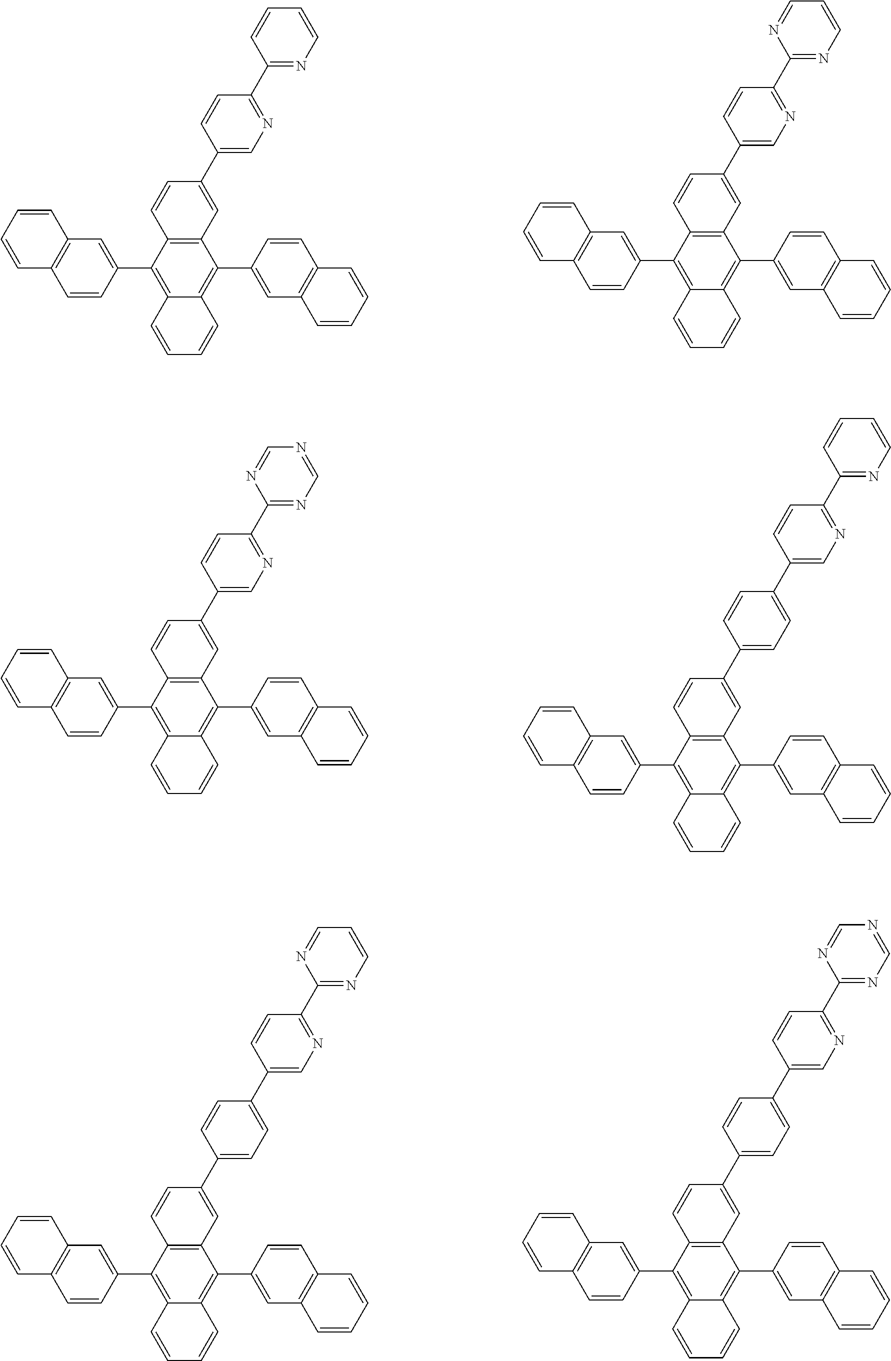

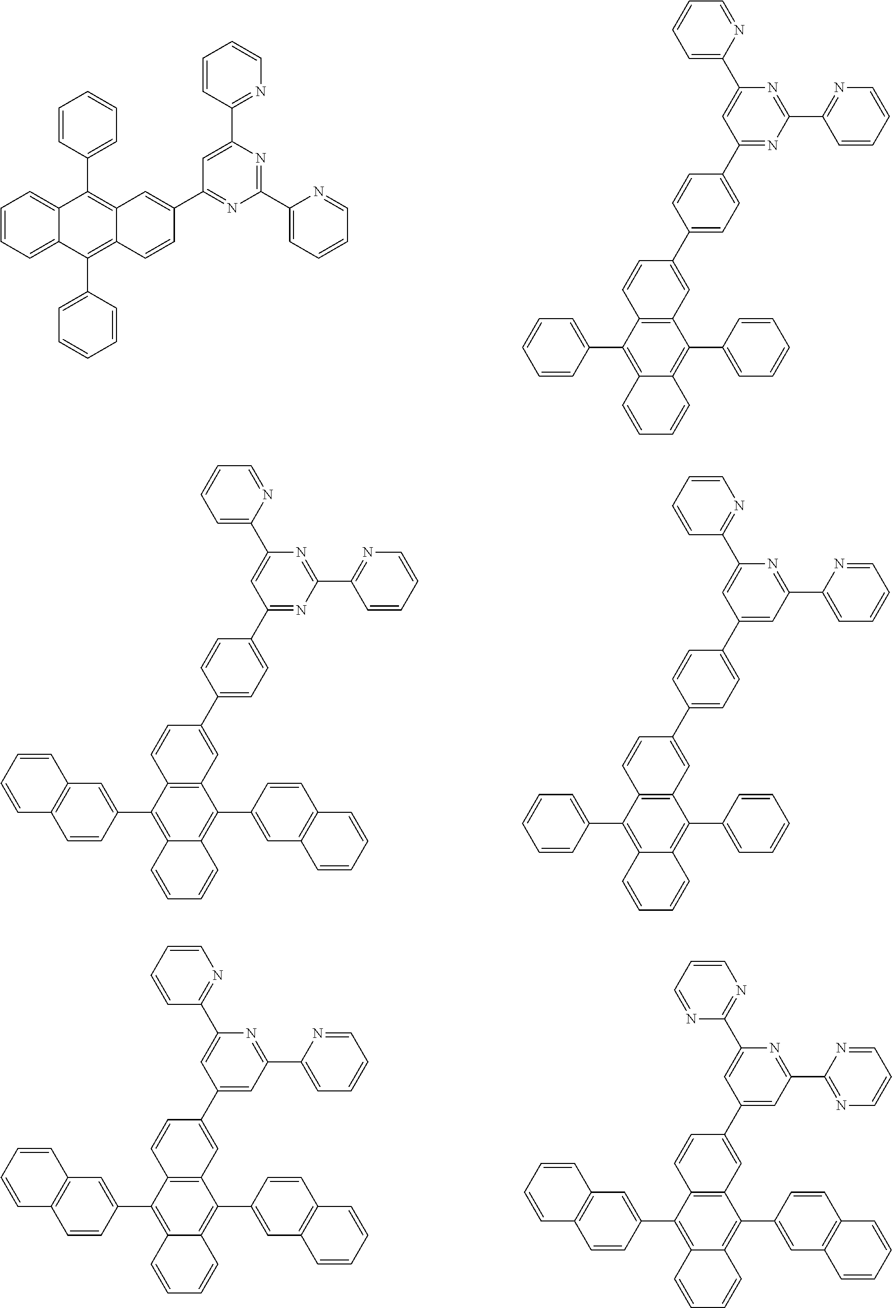

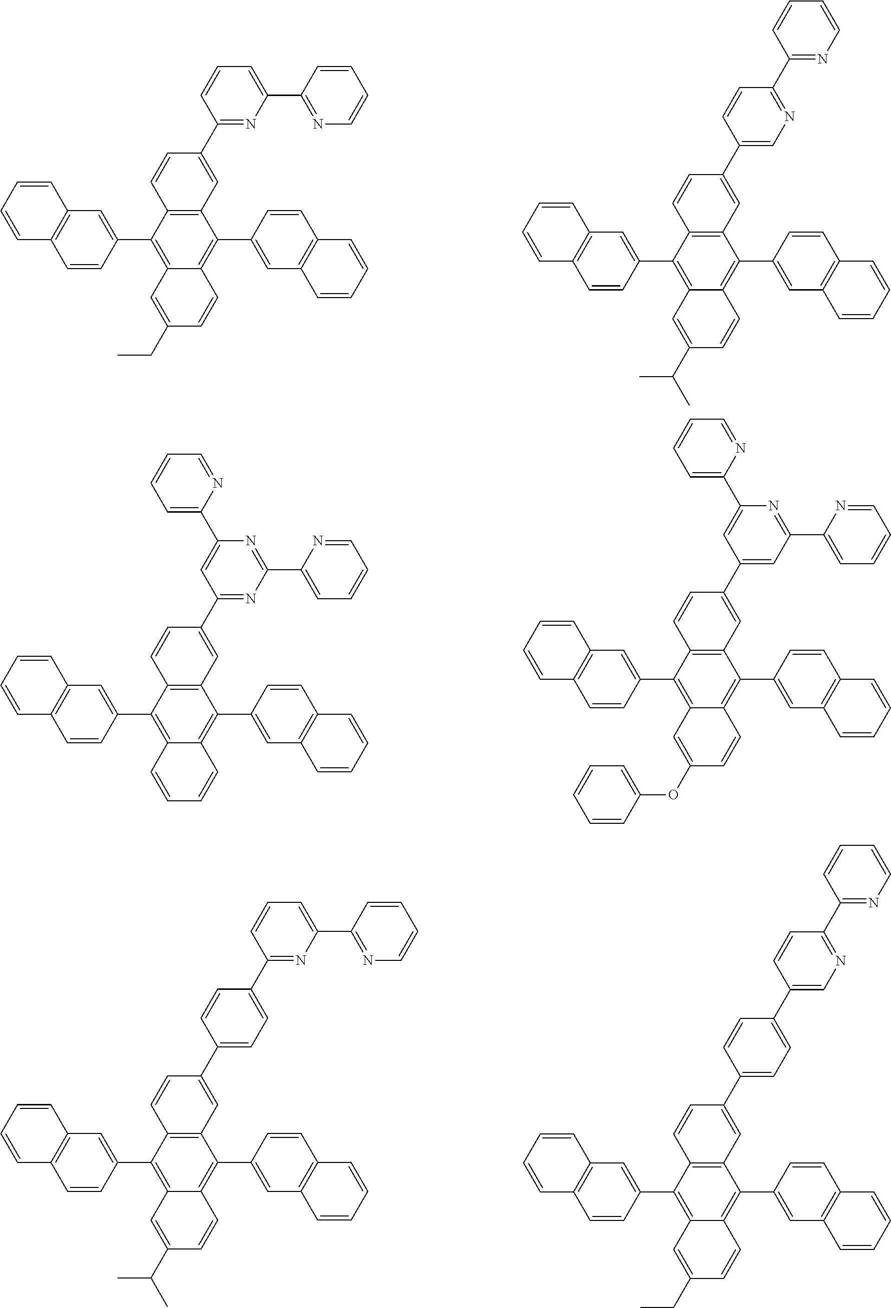

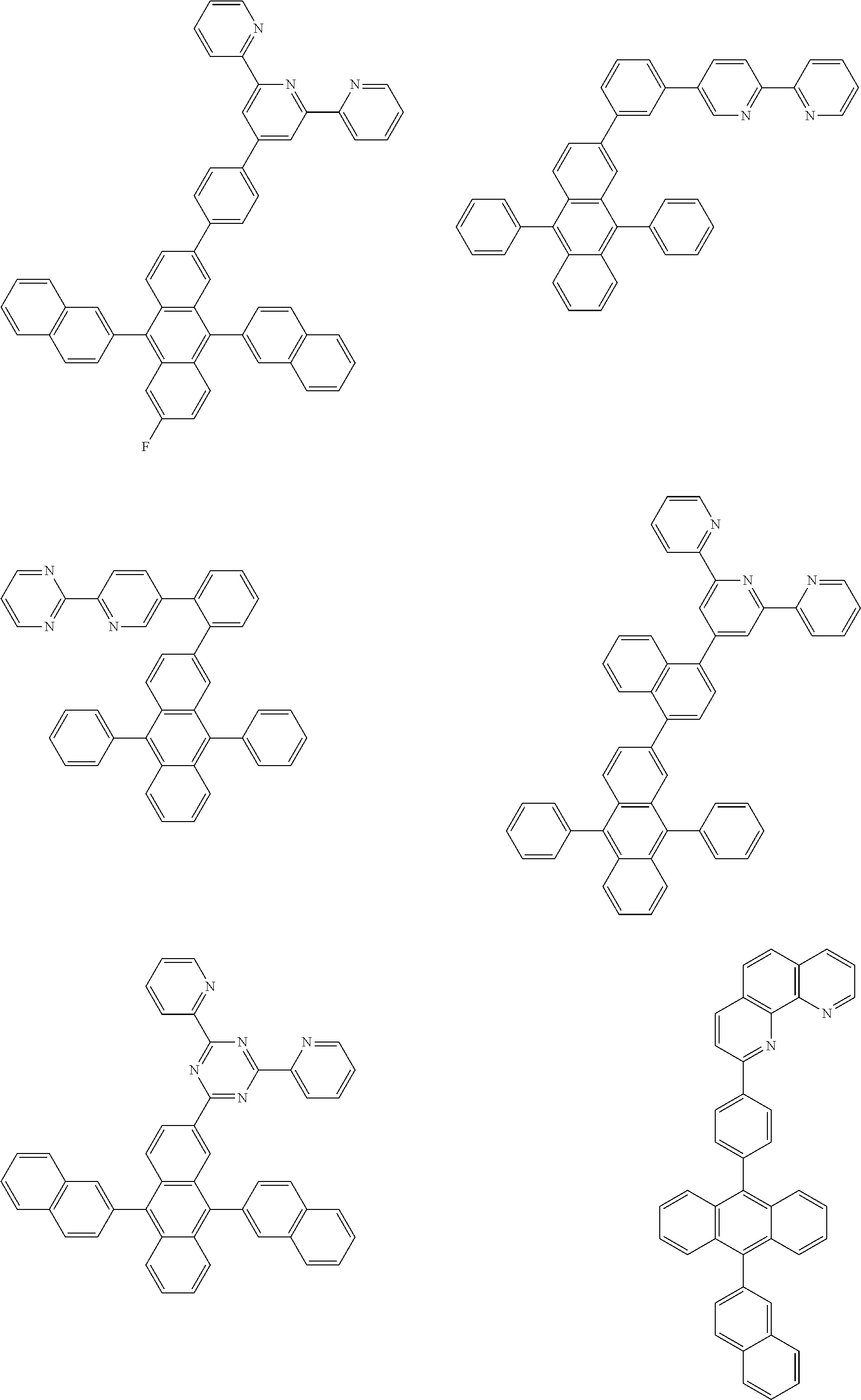

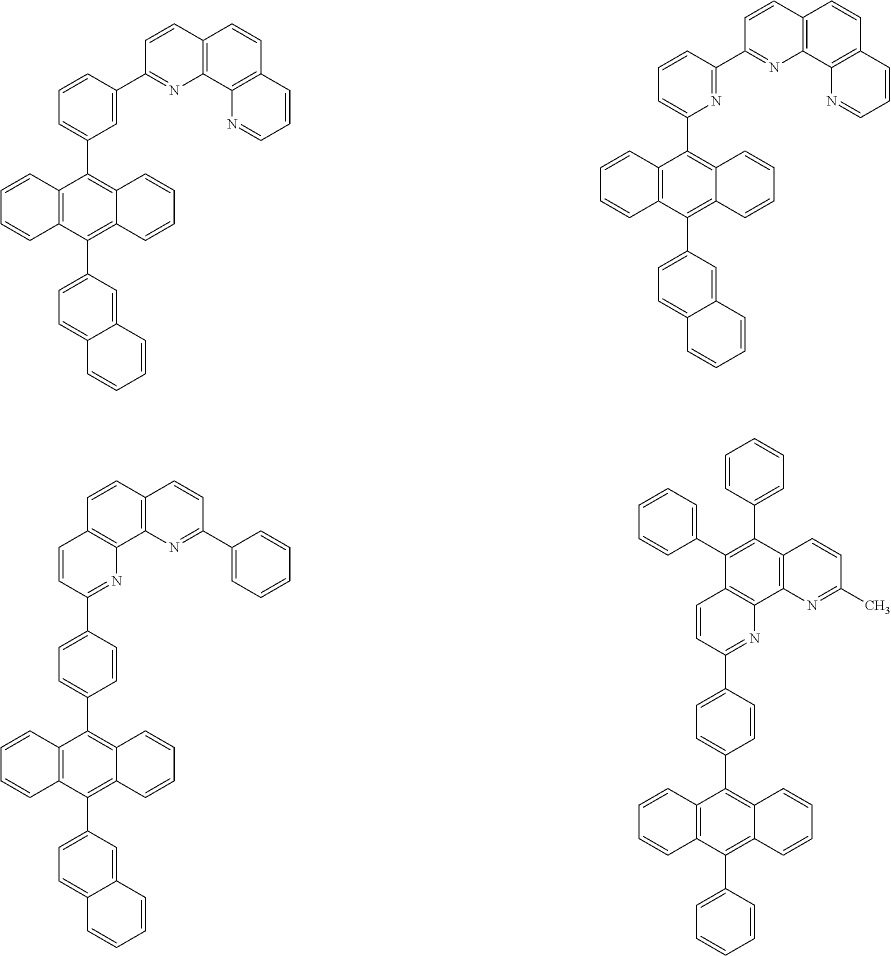 [SMILES: c1cc(-c2ccc3ccc4cccnc4c3n2)cc(-c2c3ccccc3c(-c3ccc4ccccc4c3)c3ccccc23)c1 ; c1cc(-c2ccc3ccc4cccnc4c3n2)nc(-c2c3ccccc3c(-c3ccc4ccccc4c3)c3ccccc23)c1 ; c1ccc(-c2ccc3ccc4ccc(-c5ccc(-c6c7ccccc7c(-c7ccc8ccccc8c7)c7ccccc67)cc5)nc4c3n2)cc1 ; Cc1ccc2c(-c3ccccc3)c(-c3ccccc3)c3ccc(-c4ccc(-c5c6ccccc6c(-c6ccccc6)c6ccccc56)cc4)nc3c2n1]

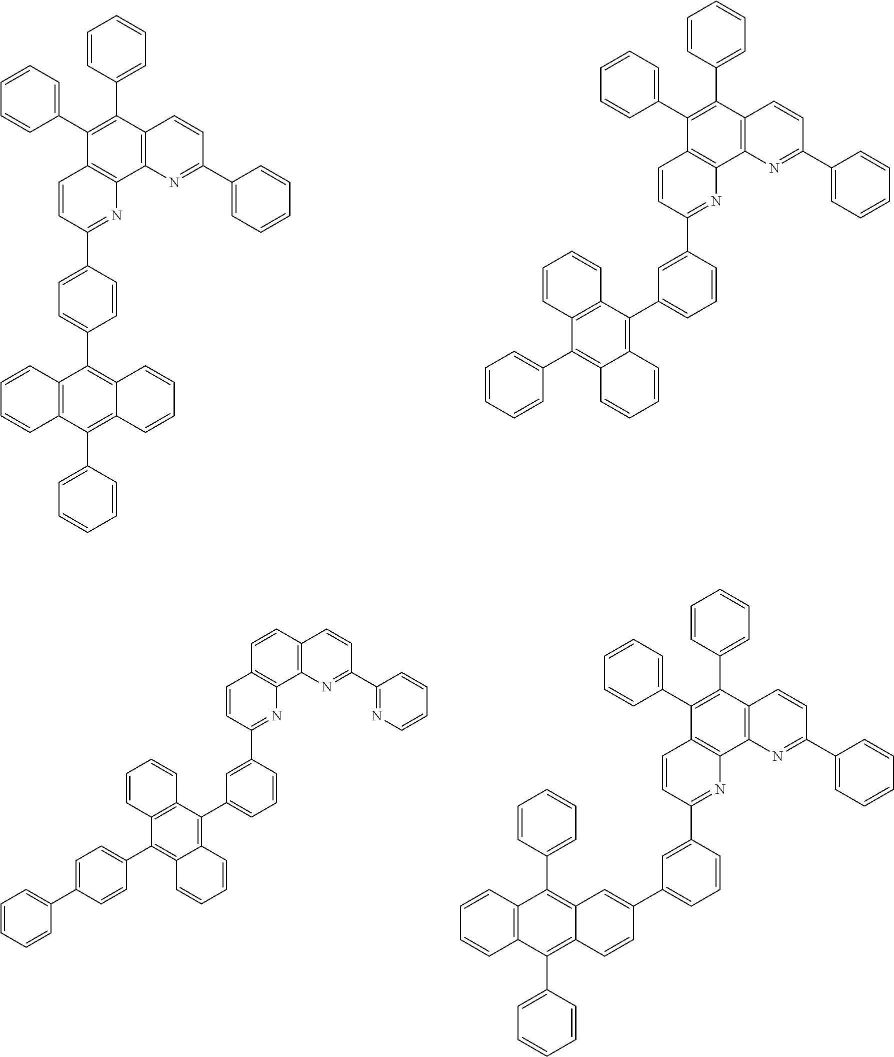 [SMILES: c1ccc(-c2ccc3c(-c4ccccc4)c(-c4ccccc4)c4ccc(-c5ccc(-c6c7ccccc7c(-c7ccccc7)c7ccccc67)cc5)nc4c3n2)cc1 ; c1ccc(-c2ccc3c(-c4ccccc4)c(-c4ccccc4)c4ccc(-c5cccc(-c6c7ccccc7c(-c7ccccc7)c7ccccc67)c5)nc4c3n2)cc1 ; c1ccc(-c2ccc(-c3c4ccccc4c(-c4cccc(-c5ccc6ccc7ccc(-c8ccccn8)nc7c6n5)c4)c4ccccc34)cc2)cc1 ; c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1]

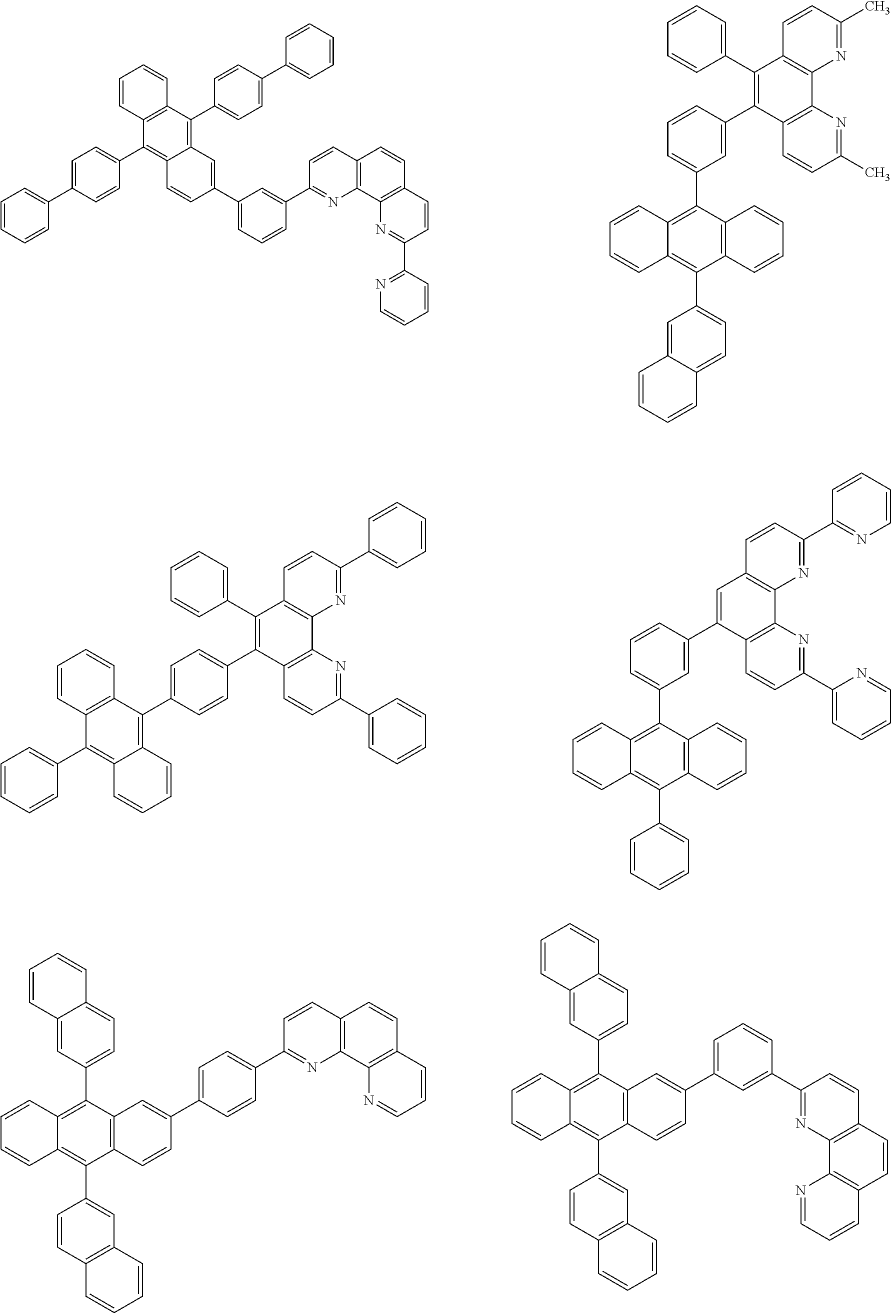

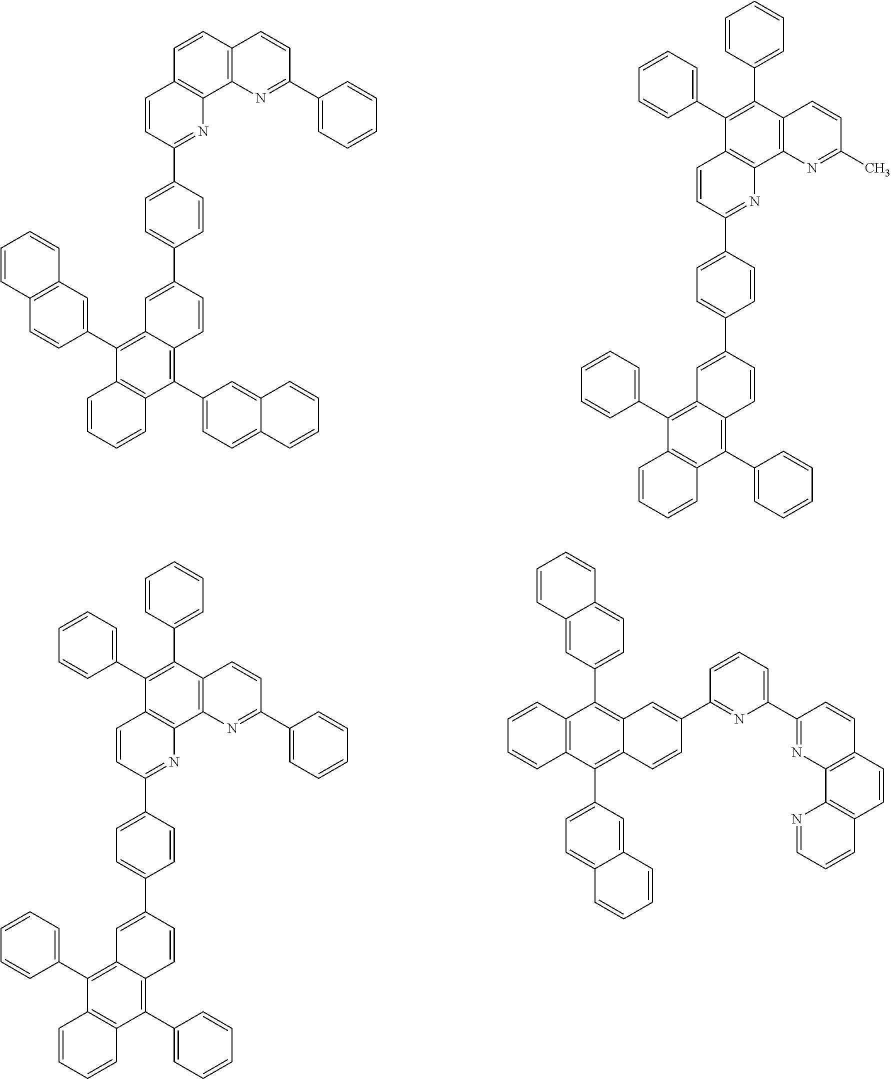 [SMILES: c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1 ; Cc1ccc2c(-c3ccccc3)c(-c3ccccc3)c3ccc(-c4ccc(-c5ccc6c(-c7ccccc7)c7ccccc7c(-c7ccccc7)c6c5)cc4)nc3c2n1 ; c1ccc(-c2ccc3c(-c4ccccc4)c(-c4ccccc4)c4ccc(-c5ccc(-c6ccc7c(-c8ccccc8)c8ccccc8c(-c8ccccc8)c7c6)cc5)nc4c3n2)cc1 ; c1cc(-c2ccc3c(-c4ccc5ccccc5c4)c4ccccc4c(-c4ccc5ccccc5c4)c3c2)nc(-c2ccc3ccc4cccnc4c3n2)c1]

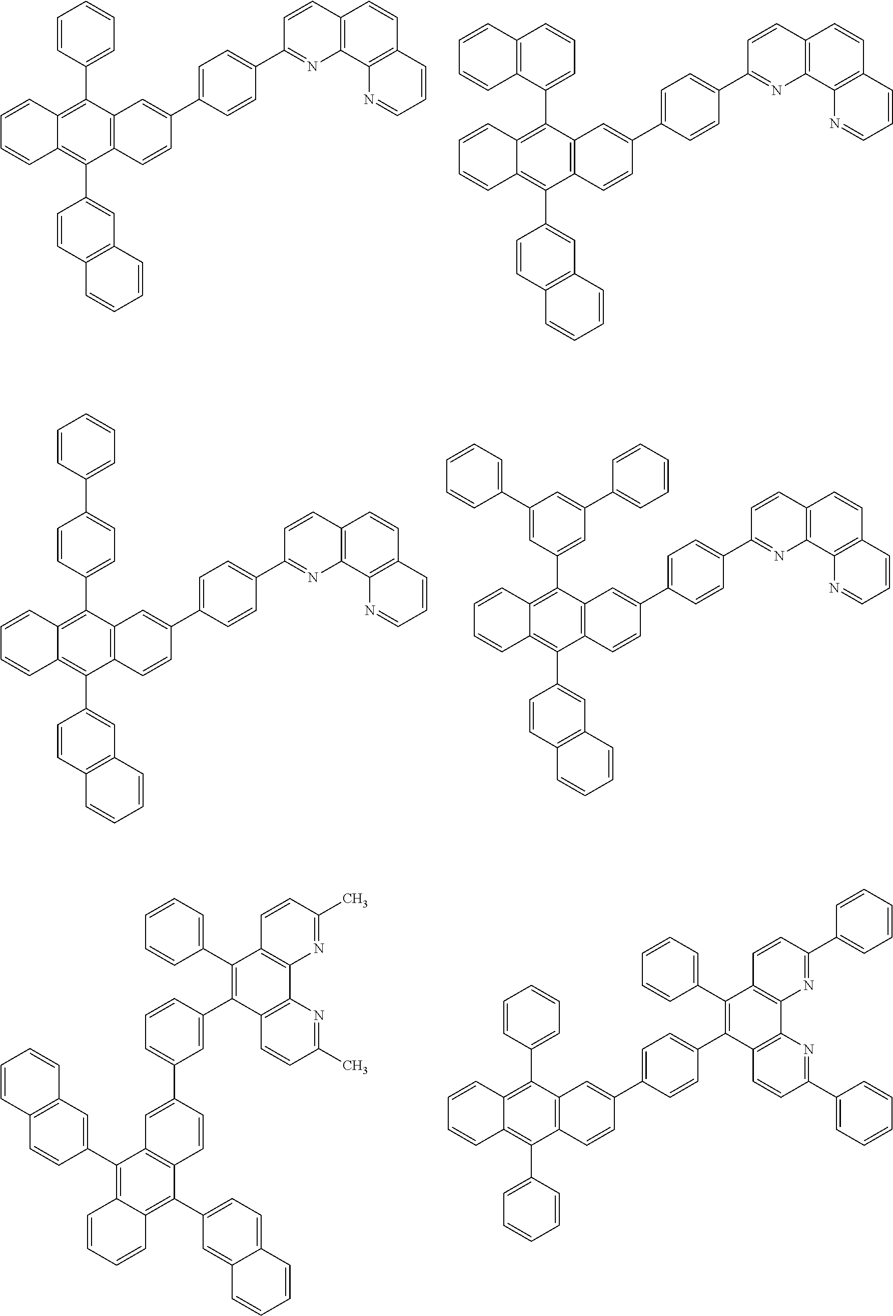

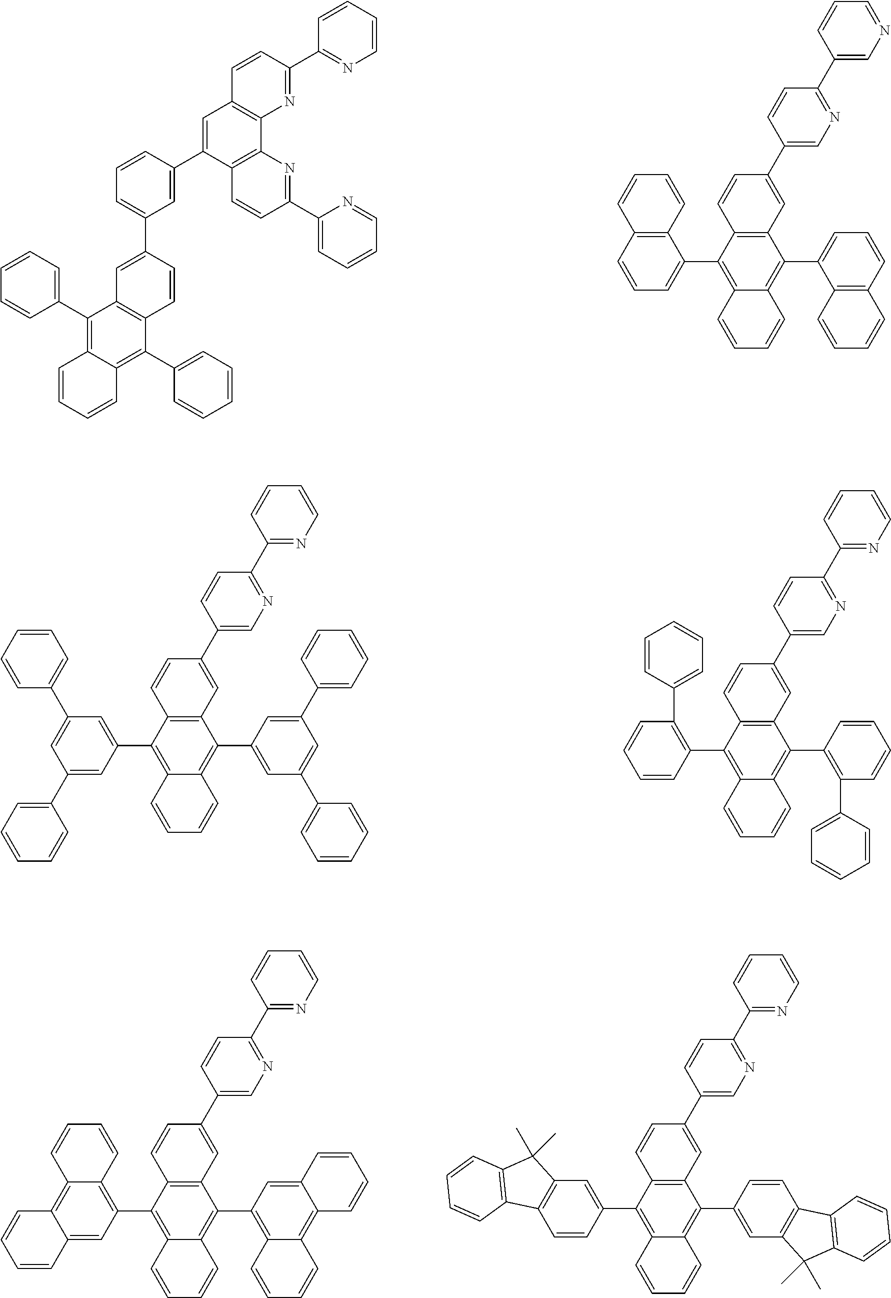

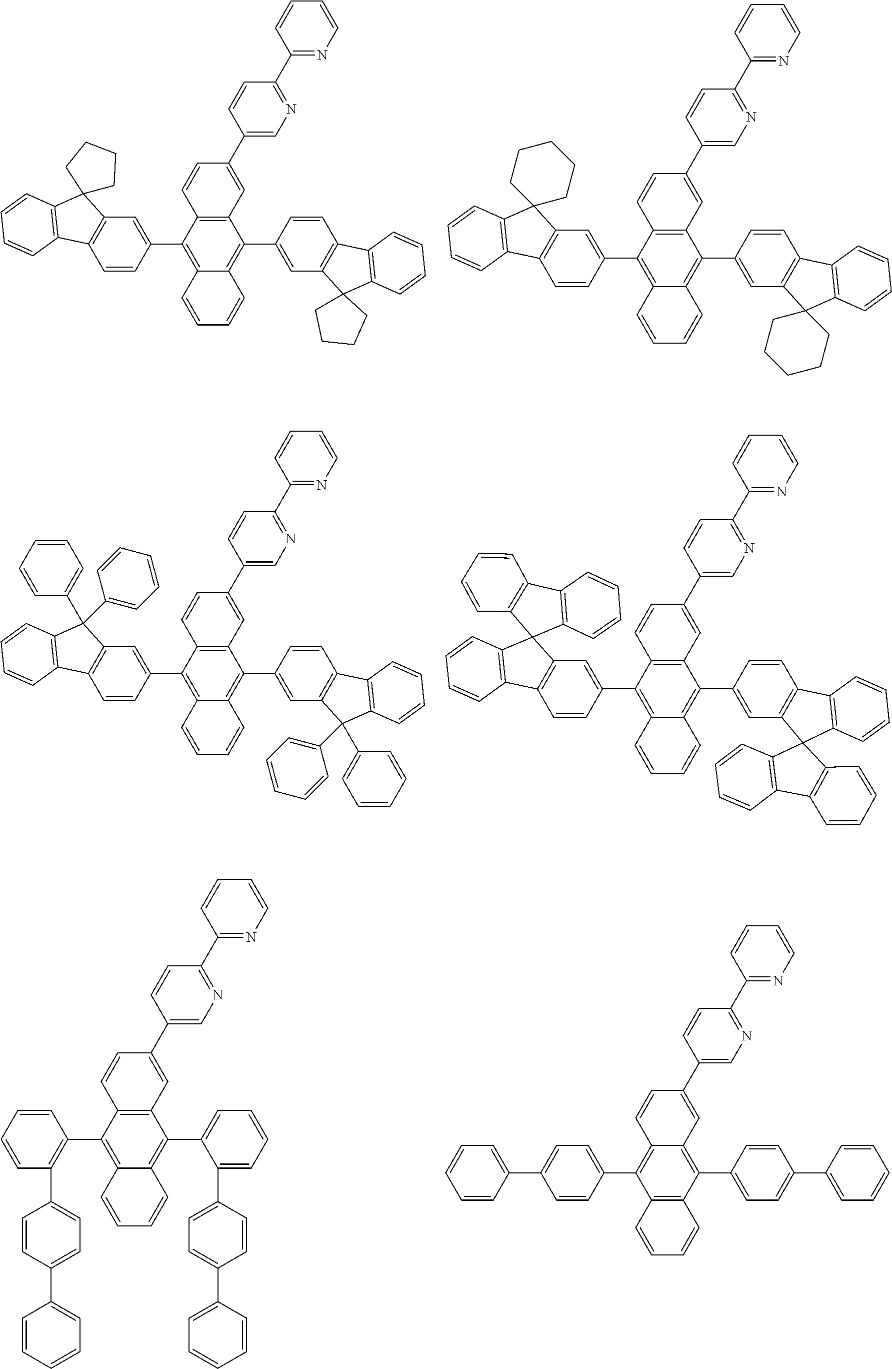

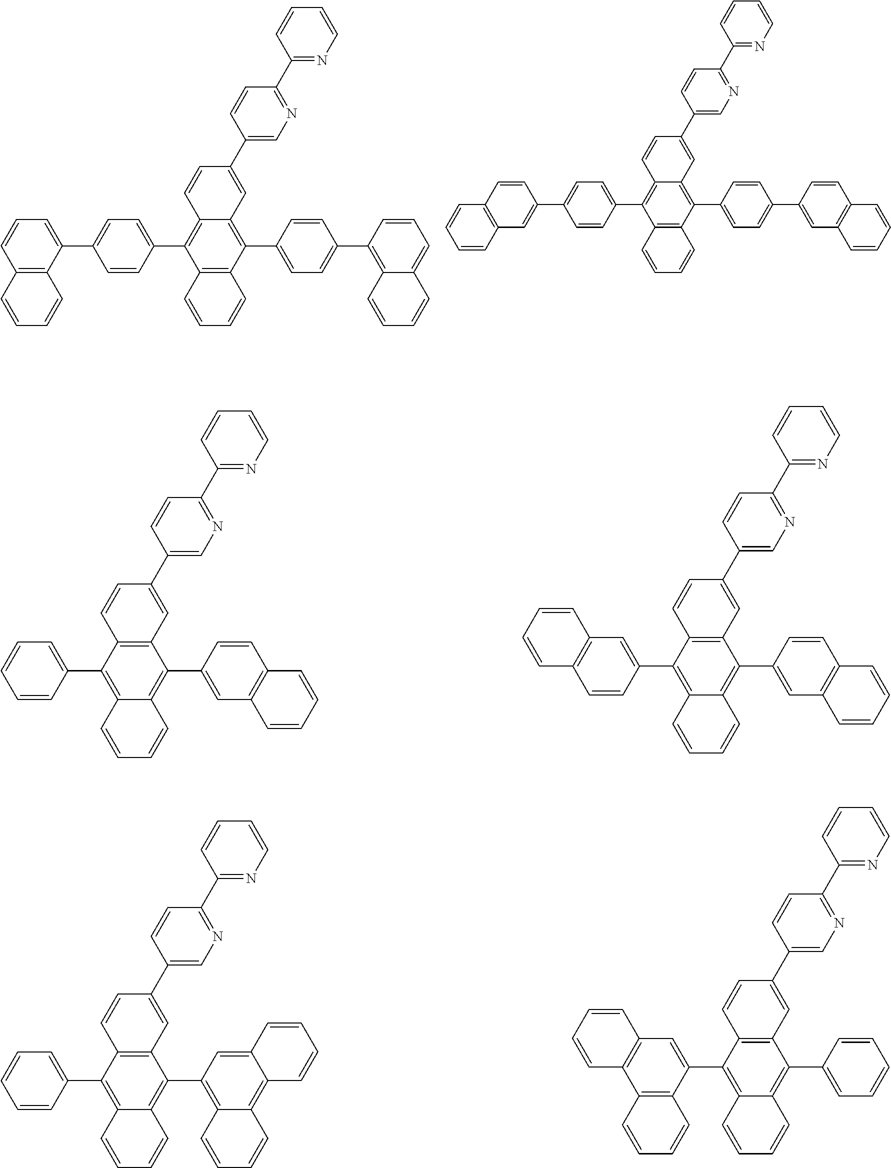

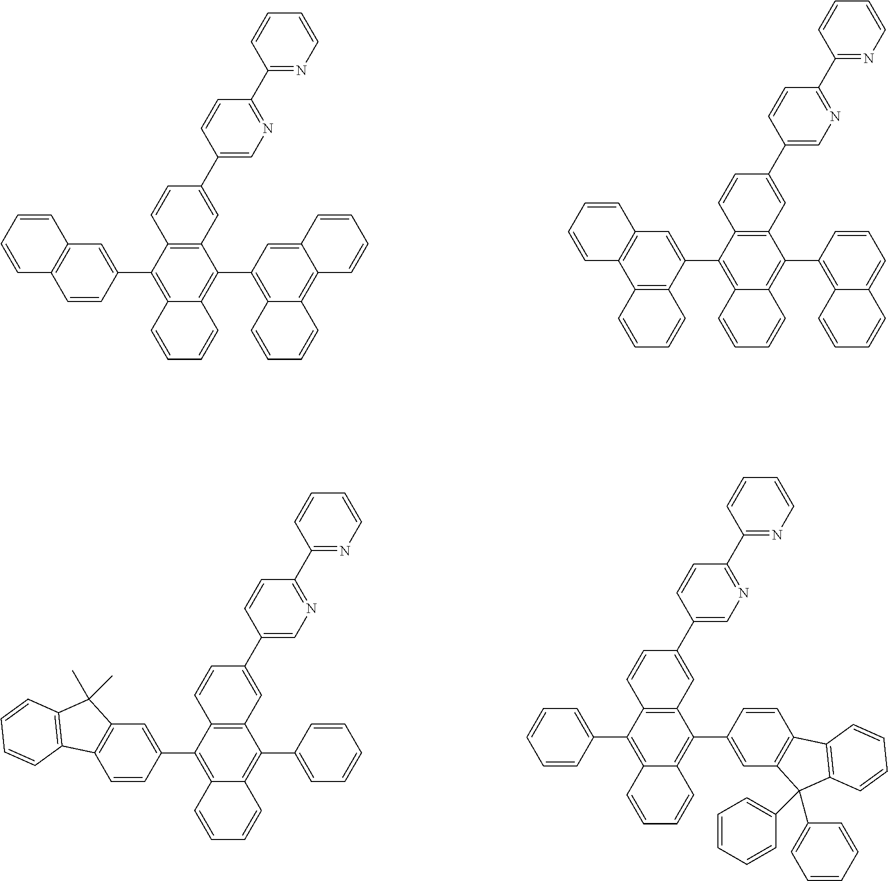 [SMILES: c1ccc(-c2ccc(-c3ccc4c(-c5ccc6ccccc6c5)c5ccccc5c(-c5cc6ccccc6c6ccccc56)c4c3)cn2)nc1 ; c1ccc(-c2ccc(-c3ccc4c(-c5cc6ccccc6c6ccccc56)c5ccccc5c(-c5cccc6ccccc56)c4c3)cn2)nc1 ; CC1(C)c2ccccc2-c2ccc(-c3c4ccccc4c(-c4ccccc4)c4cc(-c5ccc(-c6ccccn6)nc5)ccc34)cc21 ; c1ccc(-c2c3ccccc3c(-c3ccc4c(c3)C(c3ccccc3)(c3ccccc3)c3ccccc3-4)c3cc(-c4ccc(-c5ccccn5)nc4)ccc23)cc1]

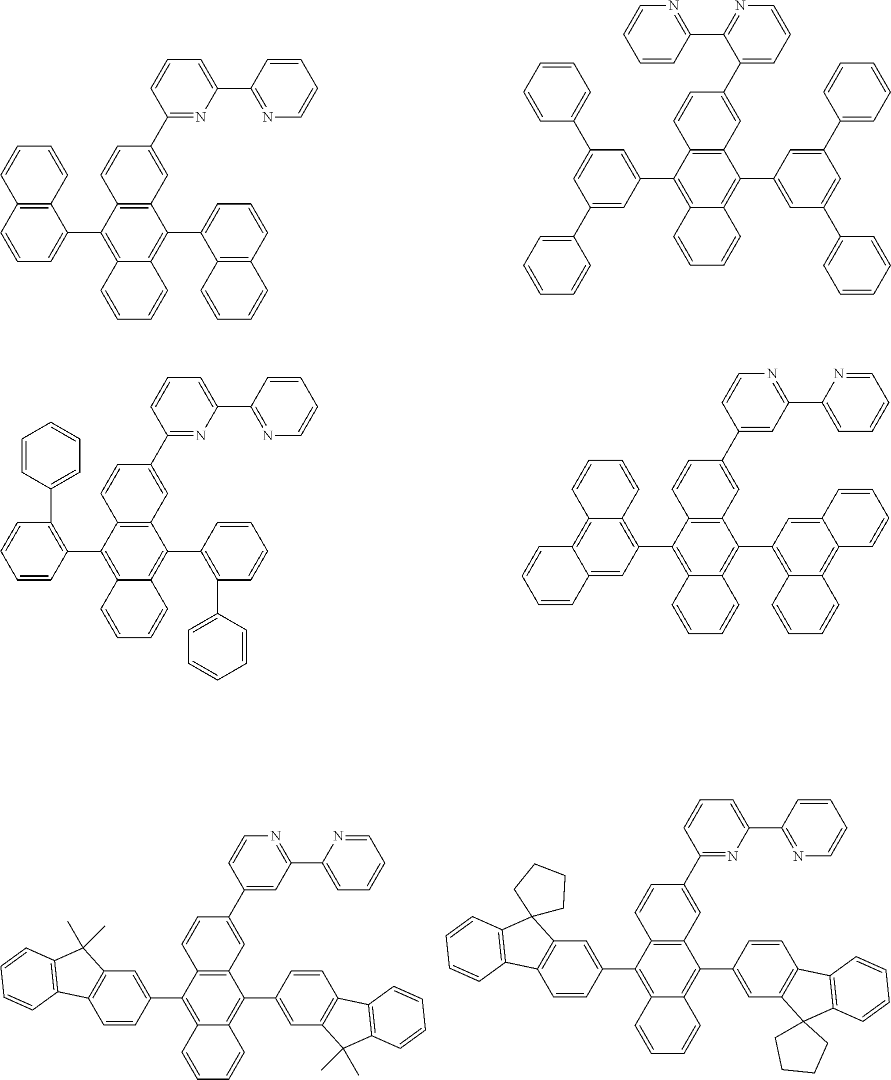

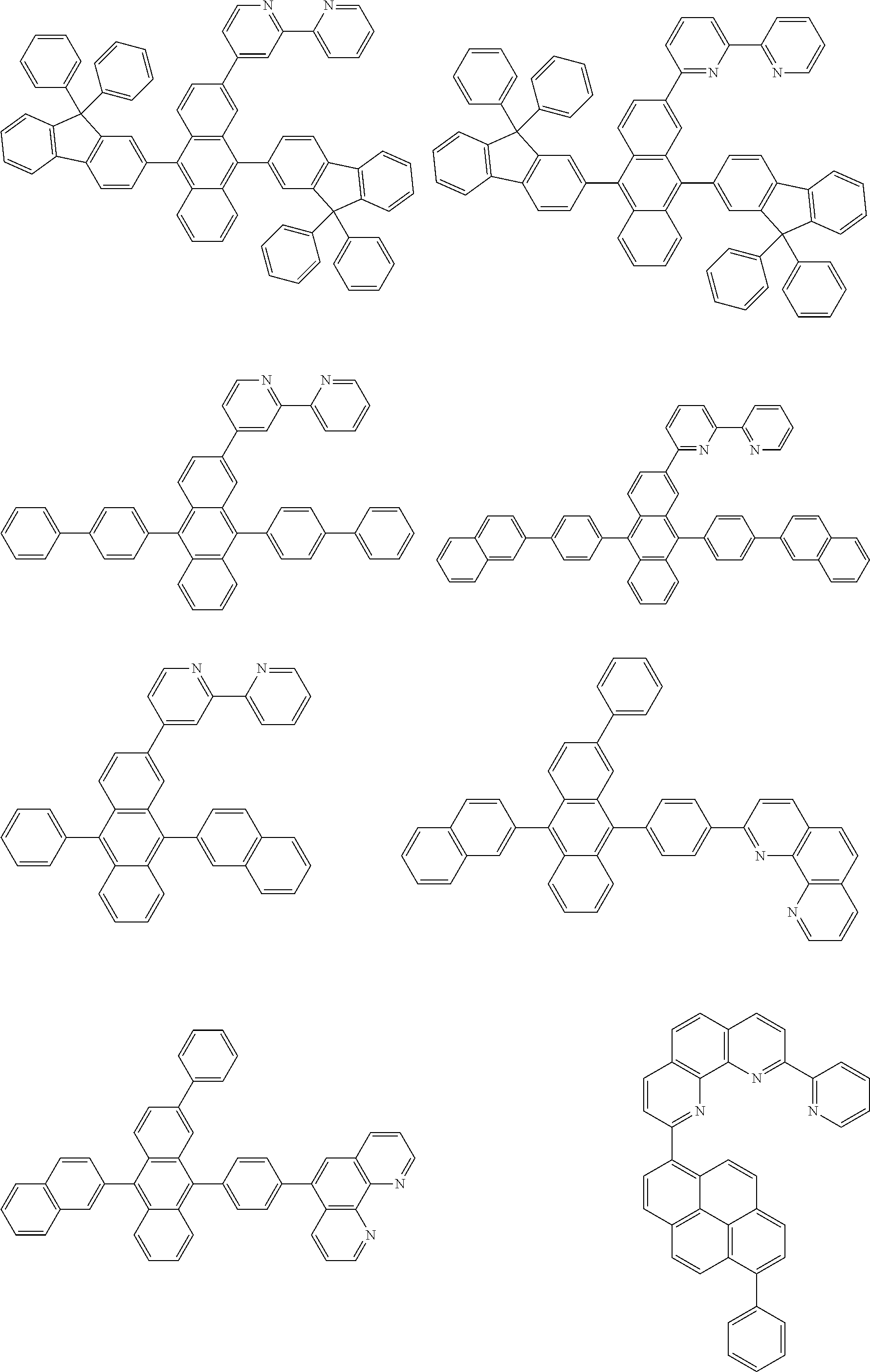

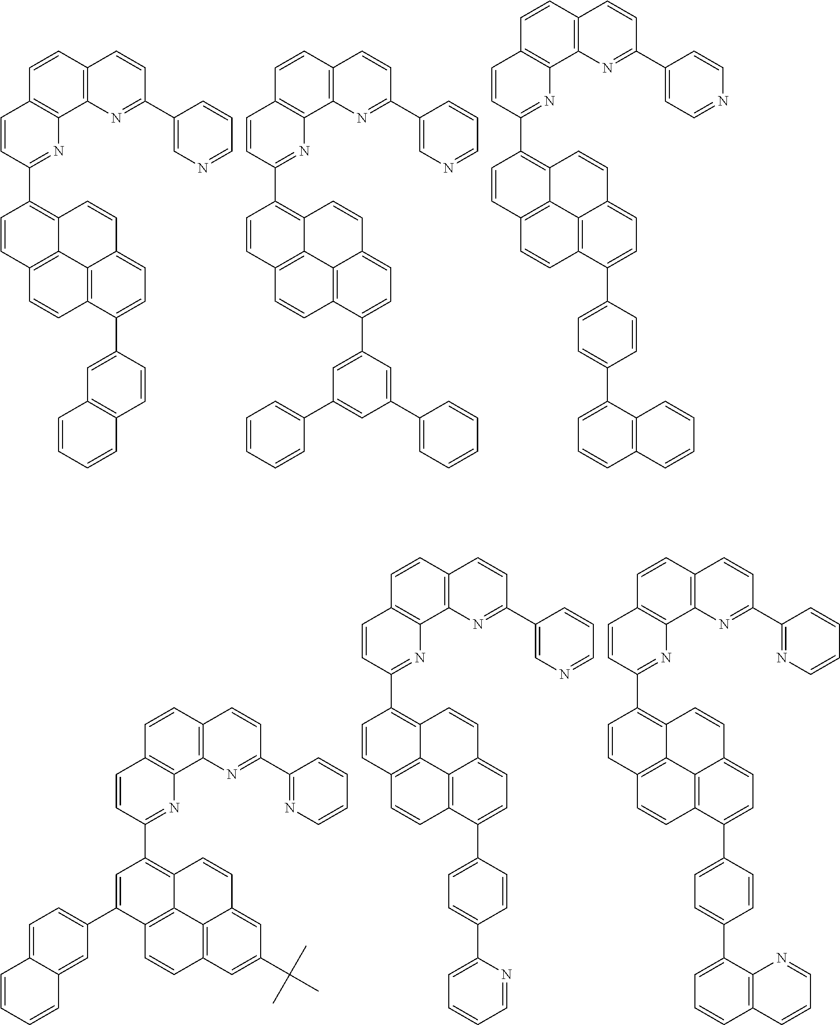

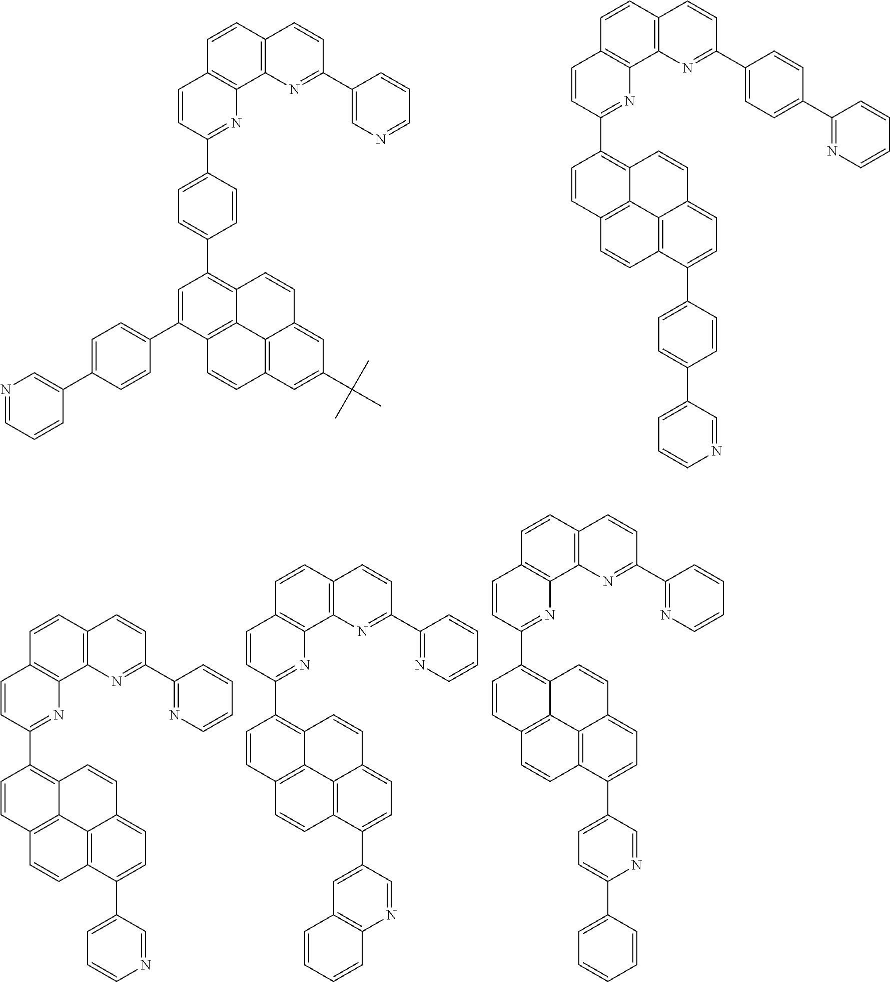

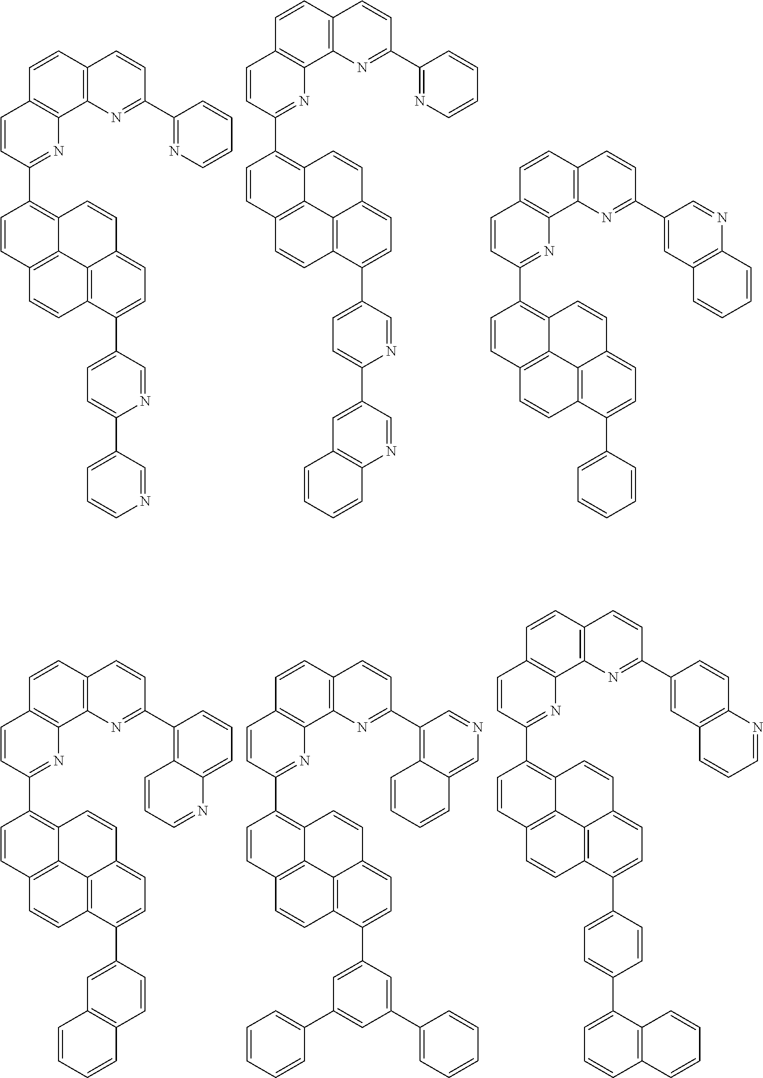

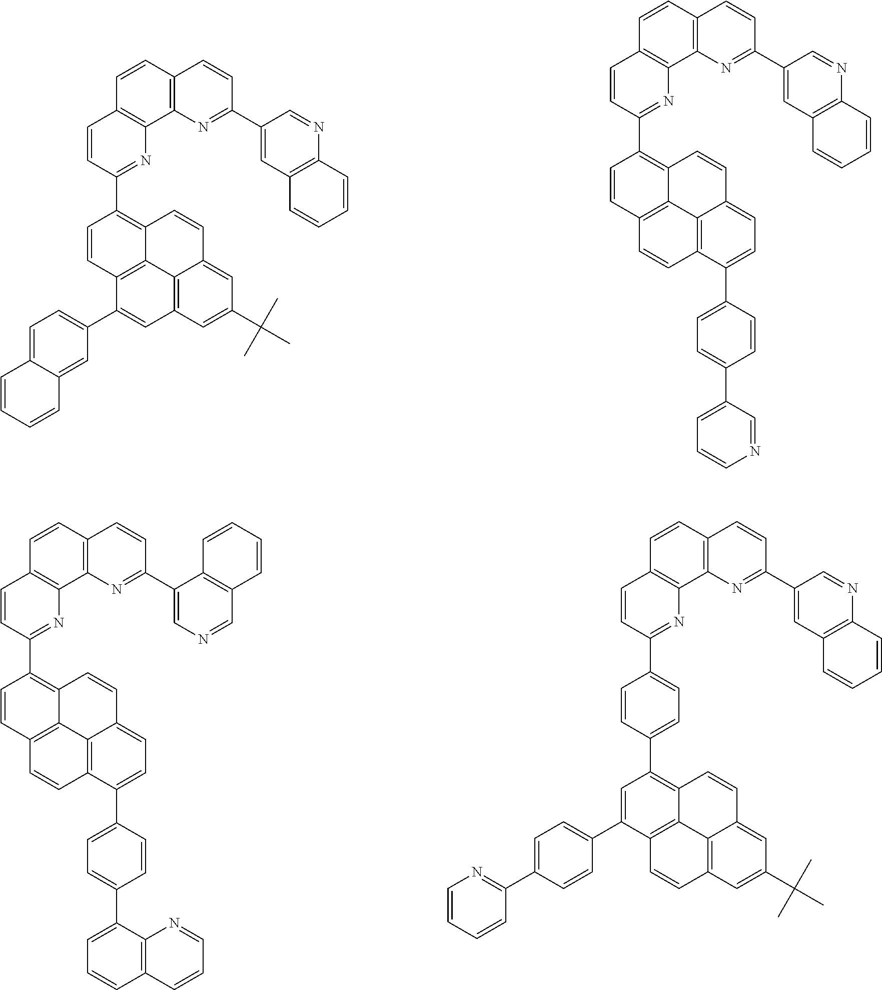 [SMILES: CC(C)(C)c1cc2ccc3c(-c4ccc5ccc6ccc(-c7cnc8ccccc8c7)nc6c5n4)ccc4c(-c5ccc6ccccc6c5)cc(c1)c2c43 ; c1cncc(-c2ccc(-c3ccc4ccc5c(-c6ccc7ccc8ccc(-c9cnc%10ccccc%10c9)nc8c7n6)ccc6ccc3c4c65)cc2)c1 ; c1ccc2c(-c3ccc4ccc5ccc(-c6ccc7ccc8c(-c9ccc(-c%10cccc%11cccnc%10%11)cc9)ccc9ccc6c7c98)nc5c4n3)cncc2c1 ; CC(C)(C)c1cc2ccc3c(-c4ccc(-c5ccccn5)cc4)cc(-c4ccc(-c5ccc6ccc7ccc(-c8cnc9ccccc9c8)nc7c6n5)cc4)c4ccc(c1)c2c34]

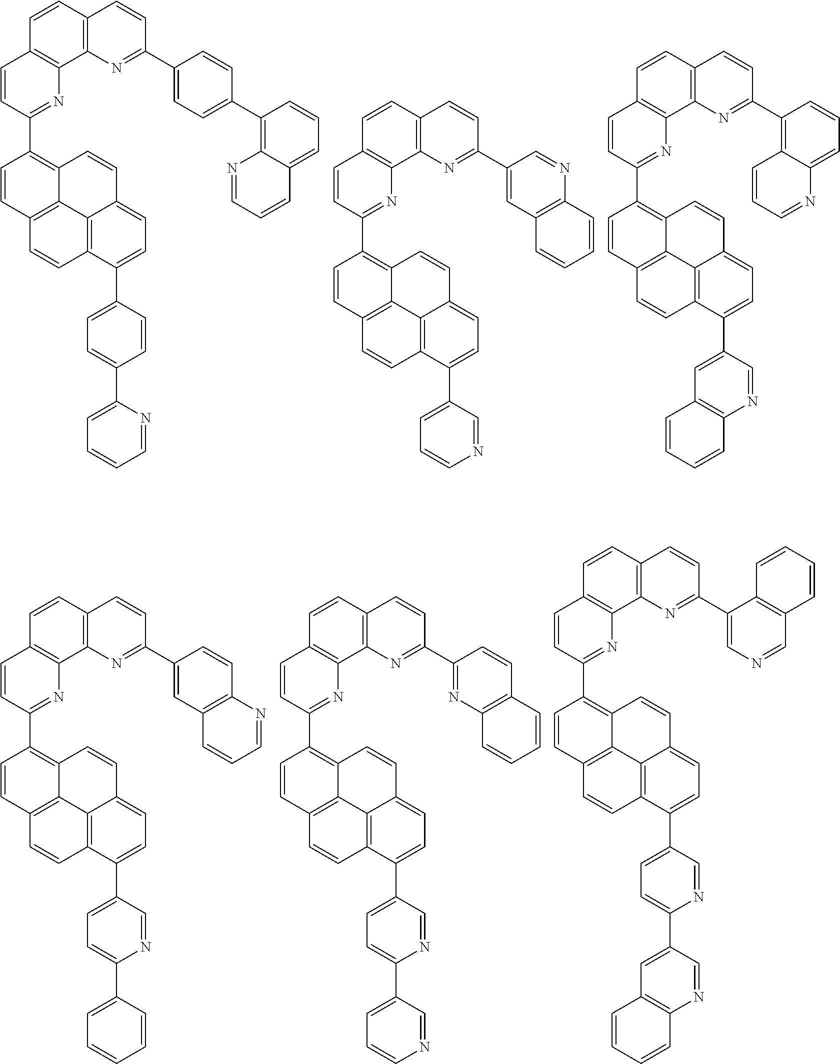

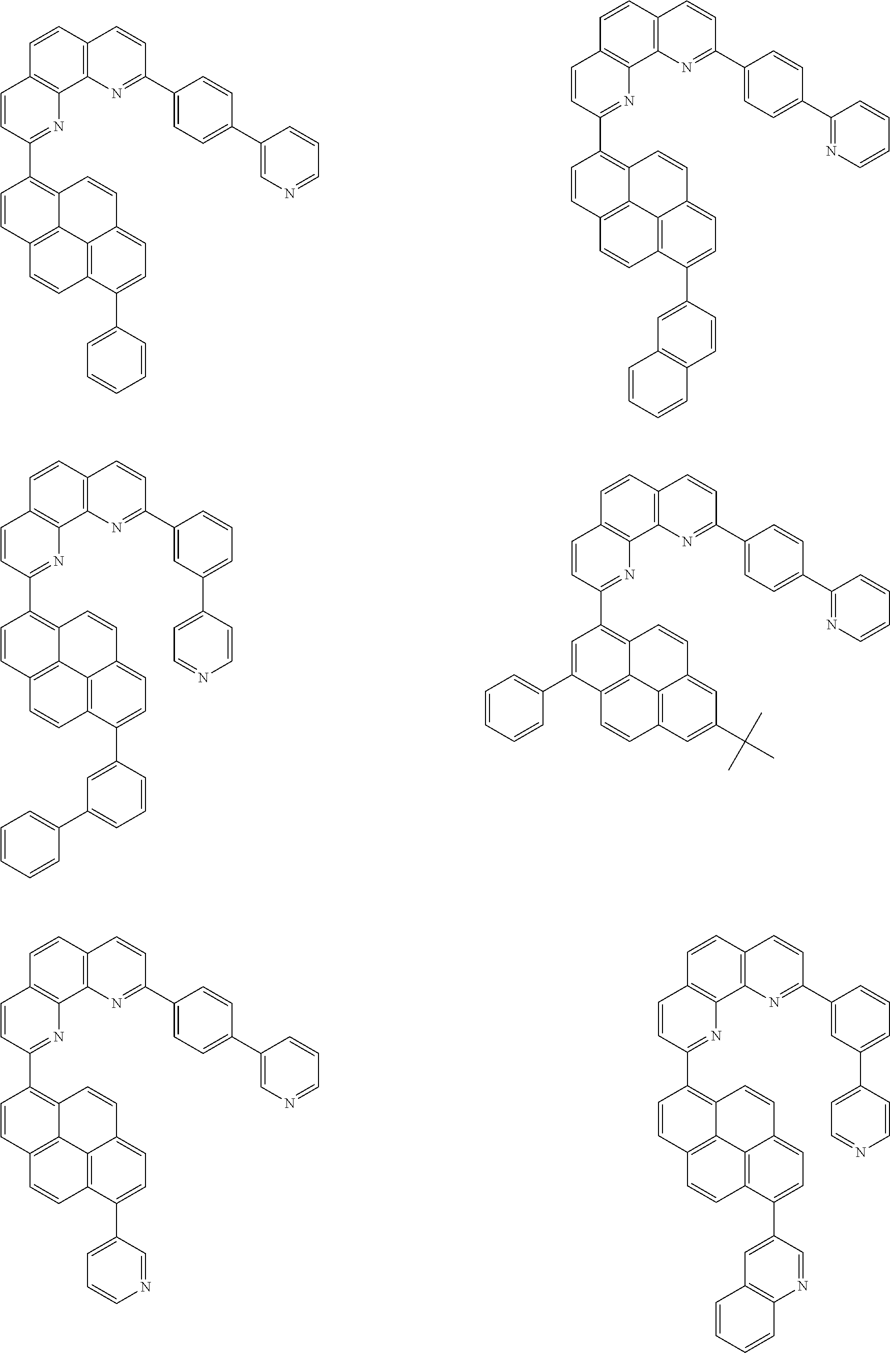

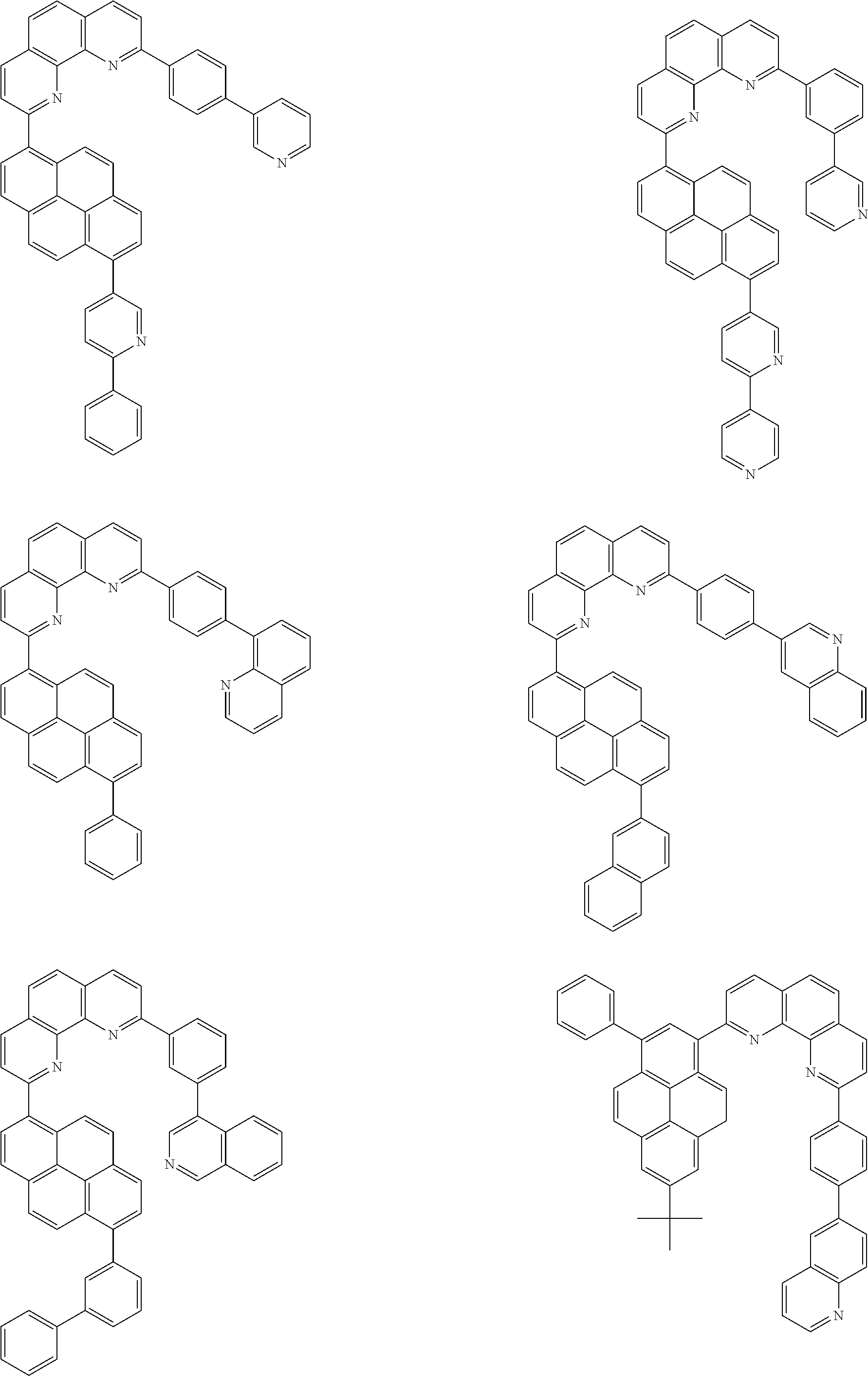

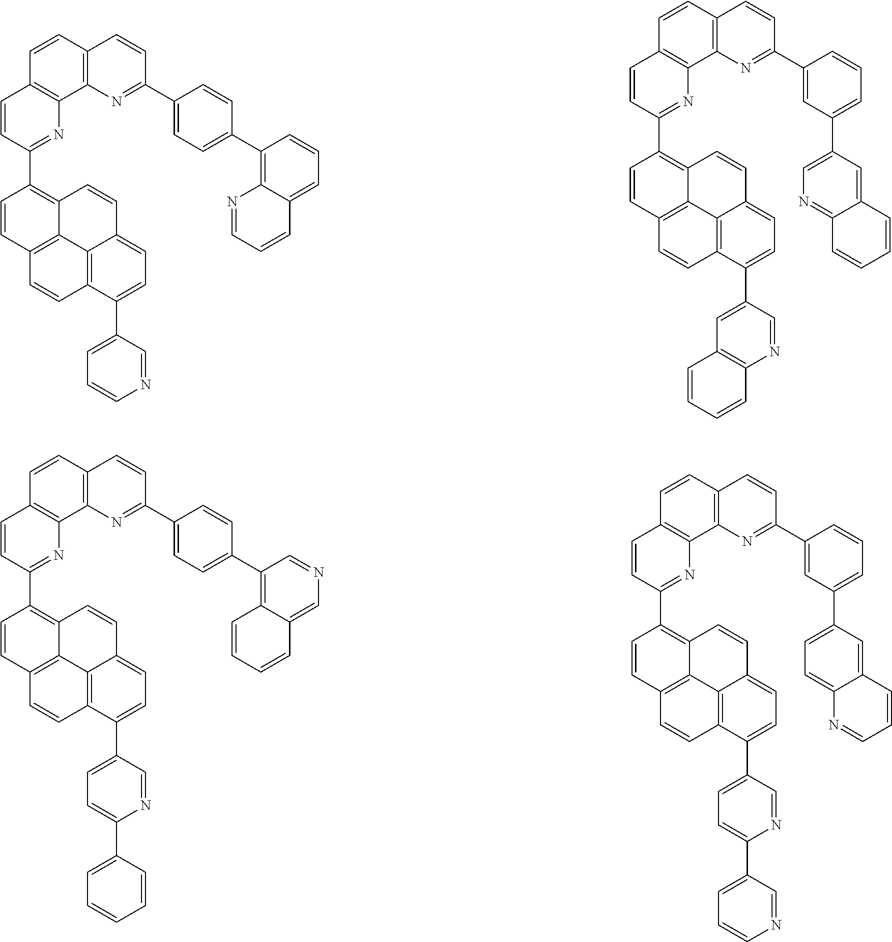 [SMILES: c1cncc(-c2ccc3ccc4c(-c5ccc6ccc7ccc(-c8ccc(-c9cccc%10cccnc9%10)cc8)nc7c6n5)ccc5ccc2c3c54)c1 ; c1cc(-c2cnc3ccccc3c2)cc(-c2ccc3ccc4ccc(-c5ccc6ccc7c(-c8cnc9ccccc9c8)ccc8ccc5c6c87)nc4c3n2)c1 ; c1ccc(-c2ccc(-c3ccc4ccc5c(-c6ccc7ccc8ccc(-c9ccc(-c%10cncc%11ccccc%10%11)cc9)nc8c7n6)ccc6ccc3c4c65)cn2)cc1 ; c1cncc(-c2ccc(-c3ccc4ccc5c(-c6ccc7ccc8ccc(-c9cccc(-c%10ccc%11ncccc%11c%10)c9)nc8c7n6)ccc6ccc3c4c65)cn2)c1]

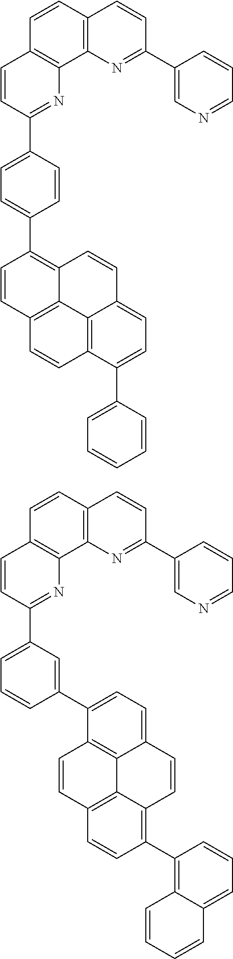

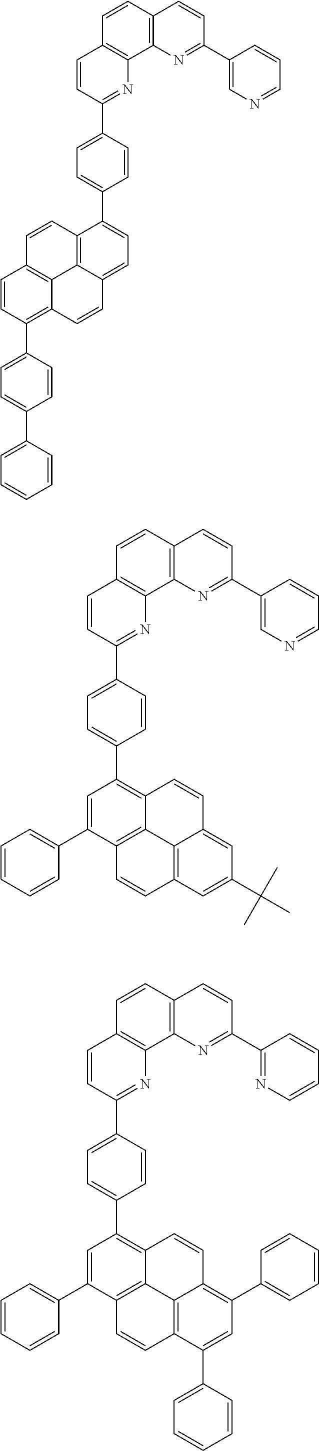 [SMILES: c1ccc(-c2ccc(-c3ccc4ccc5c(-c6ccc(-c7ccc8ccc9ccc(-c%10cccnc%10)nc9c8n7)cc6)ccc6ccc3c4c65)cc2)cc1 ; CC(C)(C)c1cc2ccc3c(-c4ccccc4)cc(-c4ccc(-c5ccc6ccc7ccc(-c8cccnc8)nc7c6n5)cc4)c4ccc(c1)c2c34 ; c1ccc(-c2cc(-c3ccccc3)c3ccc4c(-c5ccc(-c6ccc7ccc8ccc(-c9ccccn9)nc8c7n6)cc5)cc(-c5ccccc5)c5ccc2c3c54)cc1]

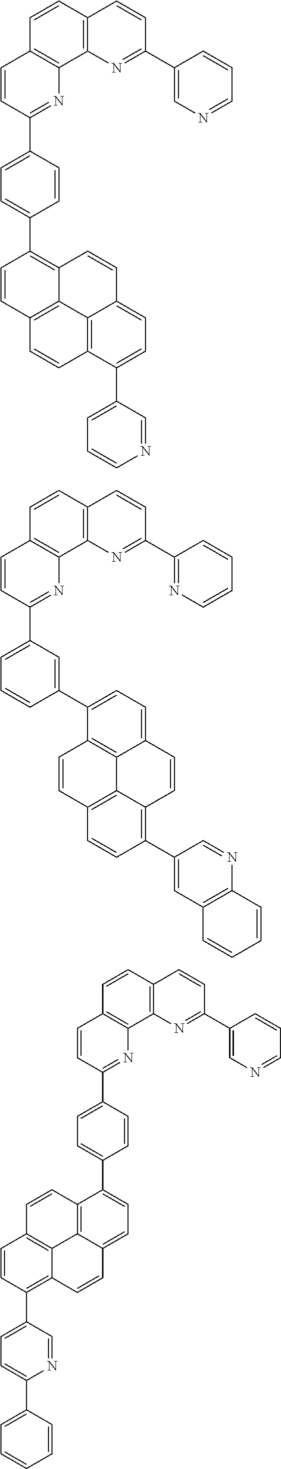

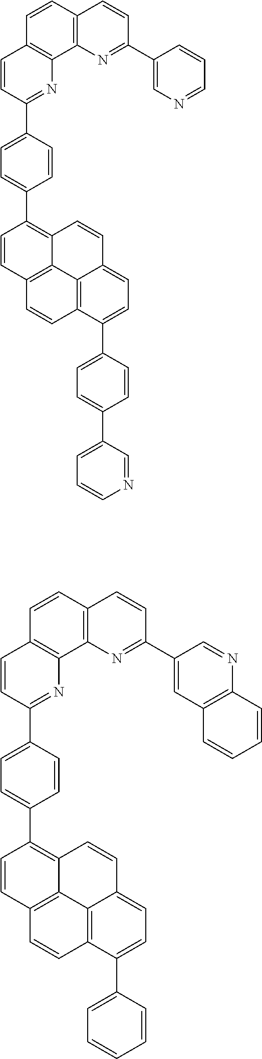 [SMILES: c1cncc(-c2ccc(-c3ccc4ccc5c(-c6ccc(-c7ccc8ccc9ccc(-c%10cccnc%10)nc9c8n7)cc6)ccc6ccc3c4c65)cc2)c1 ; c1ccc(-c2ccc3ccc4c(-c5ccc(-c6ccc7ccc8ccc(-c9cnc%10ccccc%10c9)nc8c7n6)cc5)ccc5ccc2c3c54)cc1]

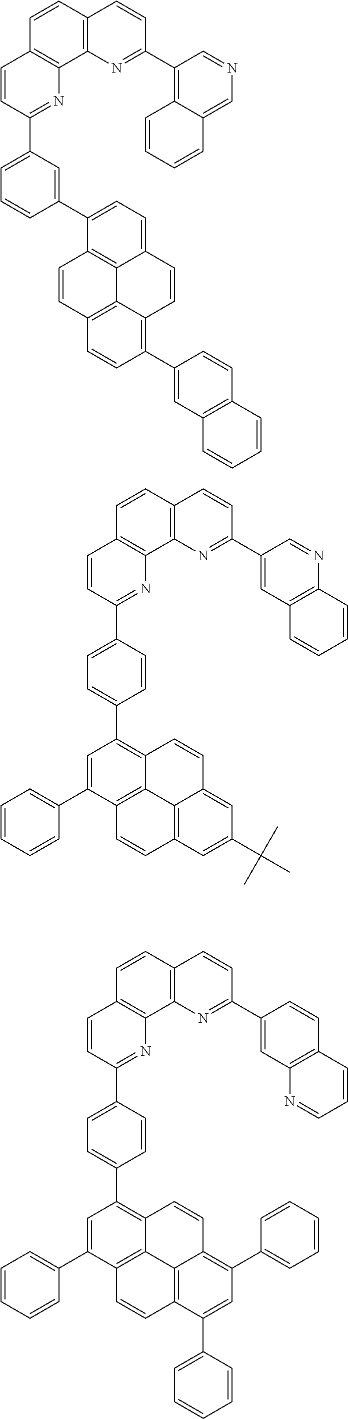

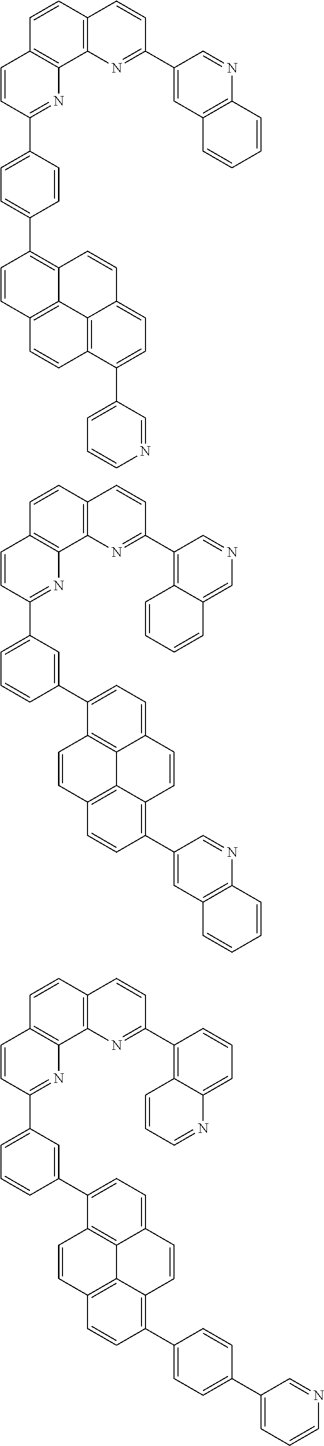

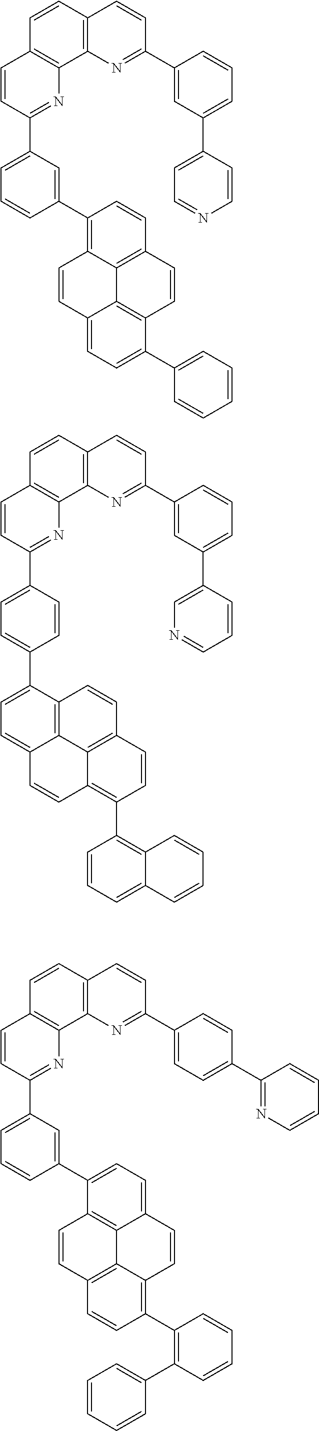 [SMILES: c1ccc(-c2ccc3ccc4c(-c5cccc(-c6ccc7ccc8ccc(-c9cccc(-c%10ccncc%10)c9)nc8c7n6)c5)ccc5ccc2c3c54)cc1 ; c1cncc(-c2cccc(-c3ccc4ccc5ccc(-c6ccc(-c7ccc8ccc9c(-c%10cccc%11ccccc%10%11)ccc%10ccc7c8c%109)cc6)nc5c4n3)c2)c1 ; c1ccc(-c2ccccc2-c2ccc3ccc4c(-c5cccc(-c6ccc7ccc8ccc(-c9ccc(-c%10ccccn%10)cc9)nc8c7n6)c5)ccc5ccc2c3c54)cc1]

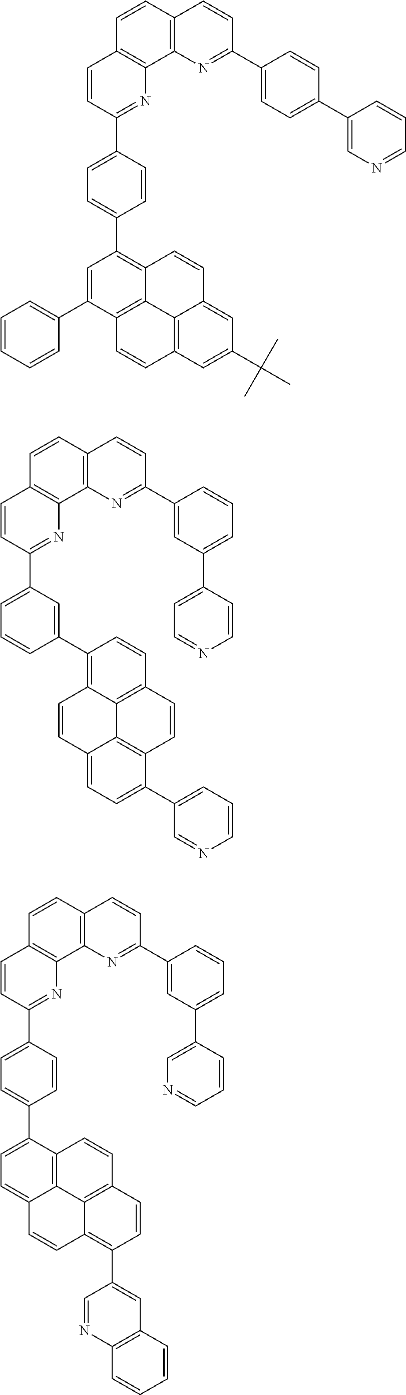 [SMILES: CC(C)(C)c1cc2ccc3c(-c4ccccc4)cc(-c4ccc(-c5ccc6ccc7ccc(-c8ccc(-c9cccnc9)cc8)nc7c6n5)cc4)c4ccc(c1)c2c34 ; c1cc(-c2ccncc2)cc(-c2ccc3ccc4ccc(-c5cccc(-c6ccc7ccc8c(-c9cccnc9)ccc9ccc6c7c98)c5)nc4c3n2)c1 ; c1cncc(-c2cccc(-c3ccc4ccc5ccc(-c6ccc(-c7ccc8ccc9c(-c%10cnc%11ccccc%11c%10)ccc%10ccc7c8c%109)cc6)nc5c4n3)c2)c1]

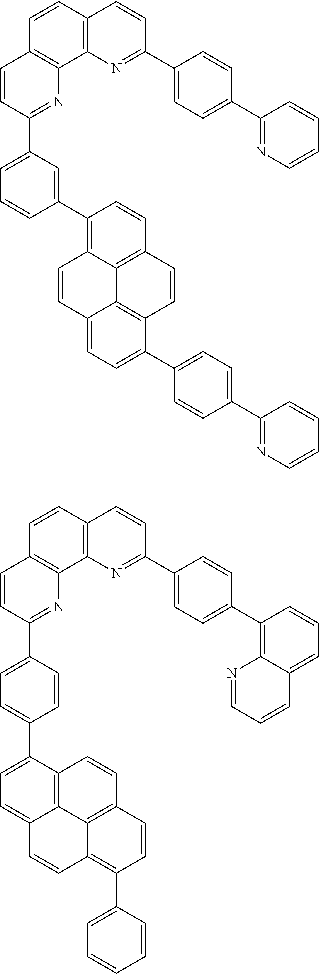 [SMILES: c1ccc(-c2ccc(-c3ccc4ccc5ccc(-c6cccc(-c7ccc8ccc9c(-c%10ccc(-c%11ccccn%11)cc%10)ccc%10ccc7c8c%109)c6)nc5c4n3)cc2)nc1 ; c1ccc(-c2ccc3ccc4c(-c5ccc(-c6ccc7ccc8ccc(-c9ccc(-c%10cccc%11cccnc%10%11)cc9)nc8c7n6)cc5)ccc5ccc2c3c54)cc1]

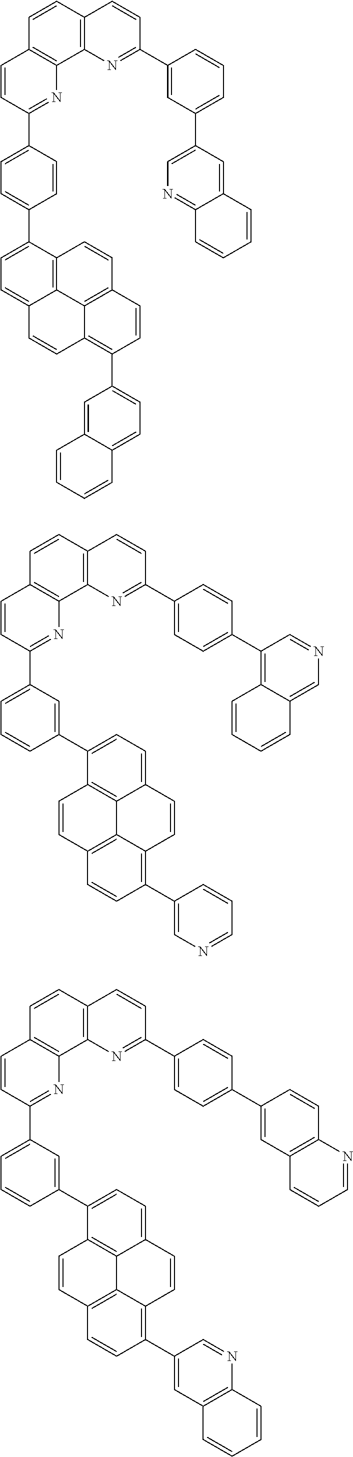

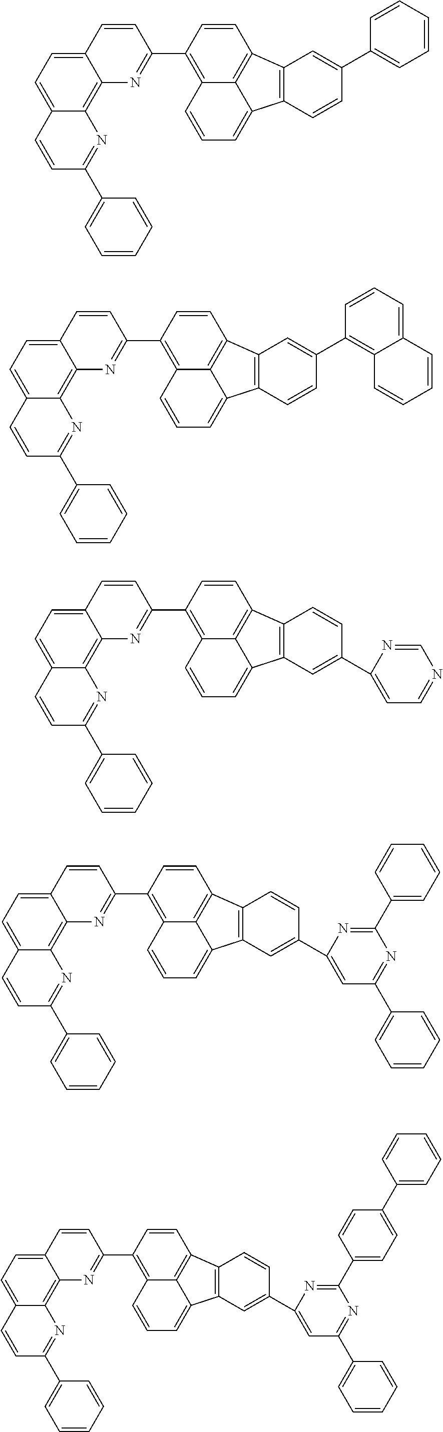

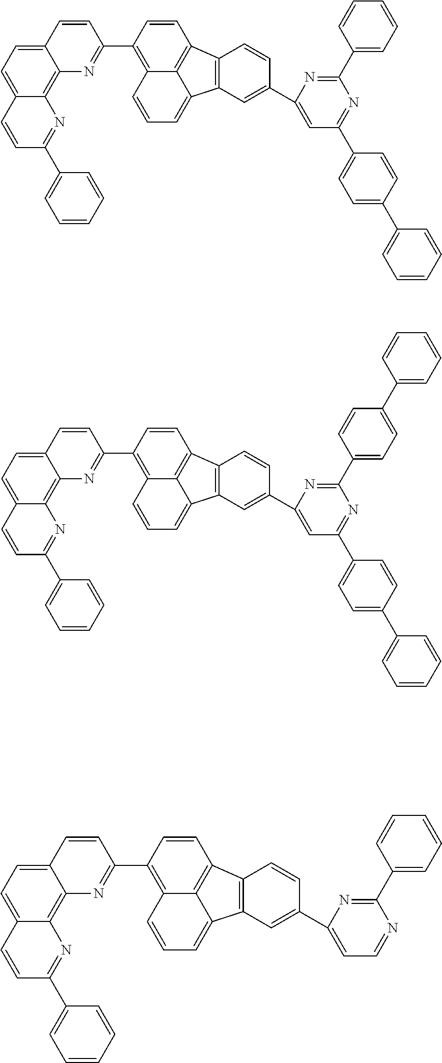 [SMILES: c1ccc(-c2ccc(-c3cc(-c4ccc5c(c4)-c4cccc6c(-c7ccc8ccc9ccc(-c%10ccccc%10)nc9c8n7)ccc-5c46)nc(-c4ccccc4)n3)cc2)cc1 ; c1ccc(-c2ccc(-c3cc(-c4ccc5c(c4)-c4cccc6c(-c7ccc8ccc9ccc(-c%10ccccc%10)nc9c8n7)ccc-5c46)nc(-c4ccc(-c5ccccc5)cc4)n3)cc2)cc1 ; c1ccc(-c2ccc3ccc4ccc(-c5ccc6c7c(cccc57)-c5cc(-c7ccnc(-c8ccccc8)n7)ccc5-6)nc4c3n2)cc1]

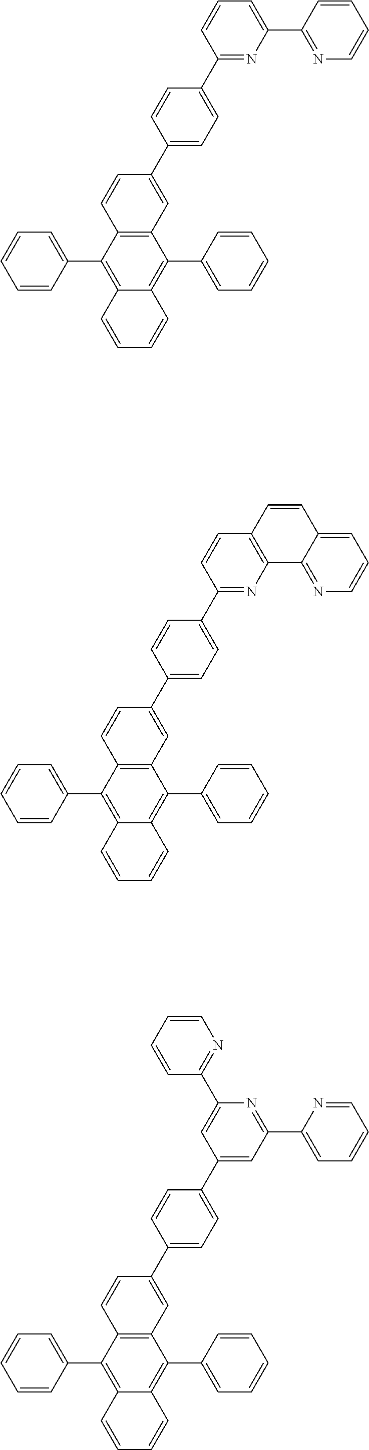 [SMILES: c1ccc(-c2c3ccccc3c(-c3ccccc3)c3cc(-c4ccc(-c5cccc(-c6ccccn6)n5)cc4)ccc23)cc1 ; c1ccc(-c2c3ccccc3c(-c3ccccc3)c3cc(-c4ccc(-c5ccc6ccc7cccnc7c6n5)cc4)ccc23)cc1 ; c1ccc(-c2c3ccccc3c(-c3ccccc3)c3cc(-c4ccc(-c5cc(-c6ccccn6)nc(-c6ccccn6)c5)cc4)ccc23)cc1]

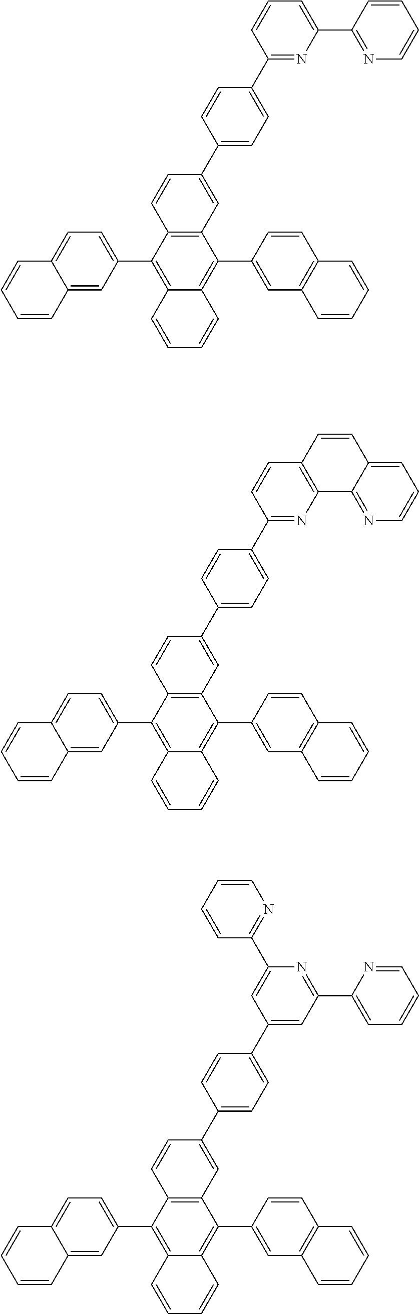 [SMILES: c1ccc(-c2cccc(-c3ccc(-c4ccc5c(-c6ccc7ccccc7c6)c6ccccc6c(-c6ccc7ccccc7c6)c5c4)cc3)n2)nc1 ; c1ccc2cc(-c3c4ccccc4c(-c4ccc5ccccc5c4)c4cc(-c5ccc(-c6ccc7ccc8cccnc8c7n6)cc5)ccc34)ccc2c1 ; c1ccc(-c2cc(-c3ccc(-c4ccc5c(-c6ccc7ccccc7c6)c6ccccc6c(-c6ccc7ccccc7c6)c5c4)cc3)cc(-c3ccccn3)n2)nc1]

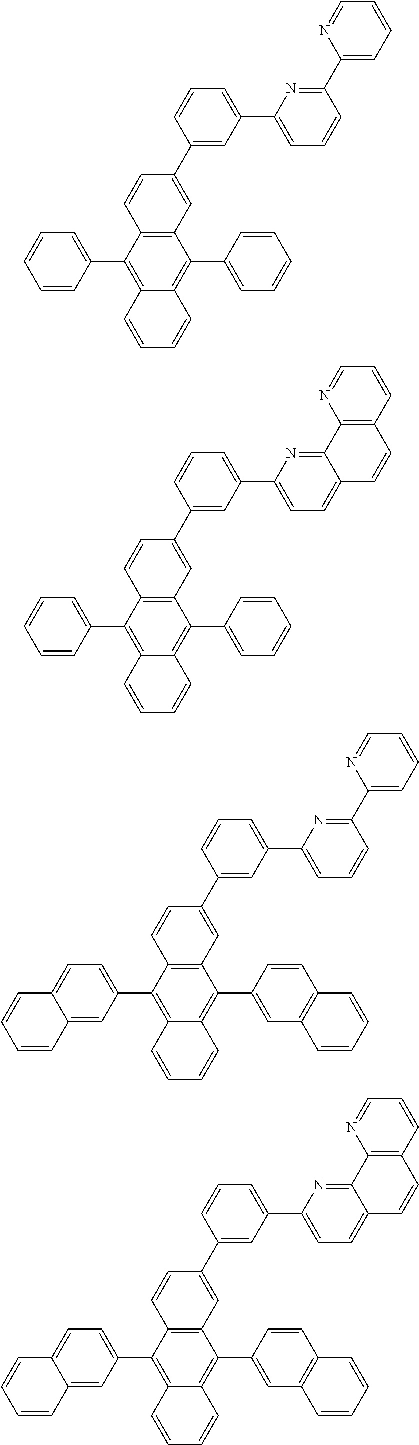 [SMILES: c1ccc(-c2c3ccccc3c(-c3ccccc3)c3cc(-c4cccc(-c5cccc(-c6ccccn6)n5)c4)ccc23)cc1 ; c1ccc(-c2c3ccccc3c(-c3ccccc3)c3cc(-c4cccc(-c5ccc6ccc7cccnc7c6n5)c4)ccc23)cc1 ; c1ccc(-c2cccc(-c3cccc(-c4ccc5c(-c6ccc7ccccc7c6)c6ccccc6c(-c6ccc7ccccc7c6)c5c4)c3)n2)nc1 ; c1cc(-c2ccc3c(-c4ccc5ccccc5c4)c4ccccc4c(-c4ccc5ccccc5c4)c3c2)cc(-c2ccc3ccc4cccnc4c3n2)c1]

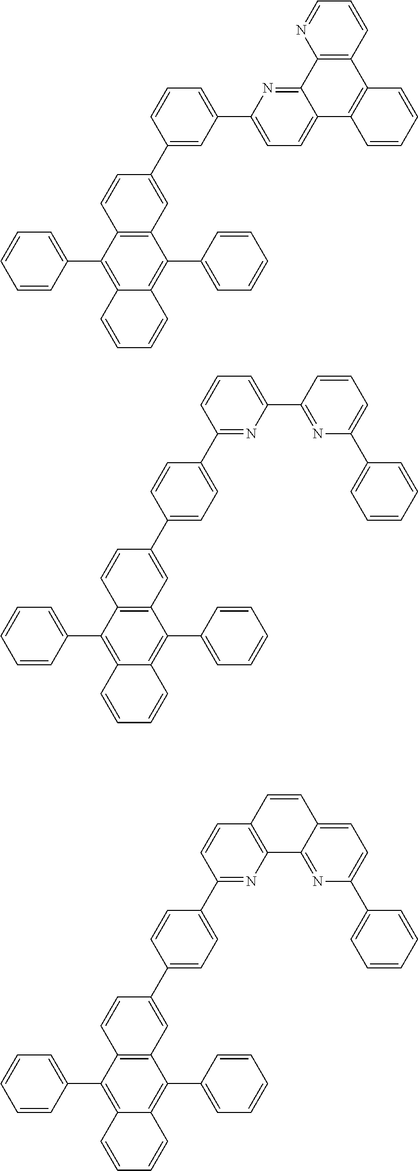 [SMILES: c1ccc(-c2c3ccccc3c(-c3ccccc3)c3cc(-c4cccc(-c5ccc6c7ccccc7c7cccnc7c6n5)c4)ccc23)cc1 ; c1ccc(-c2cccc(-c3cccc(-c4ccc(-c5ccc6c(-c7ccccc7)c7ccccc7c(-c7ccccc7)c6c5)cc4)n3)n2)cc1 ; c1ccc(-c2ccc3ccc4ccc(-c5ccc(-c6ccc7c(-c8ccccc8)c8ccccc8c(-c8ccccc8)c7c6)cc5)nc4c3n2)cc1]

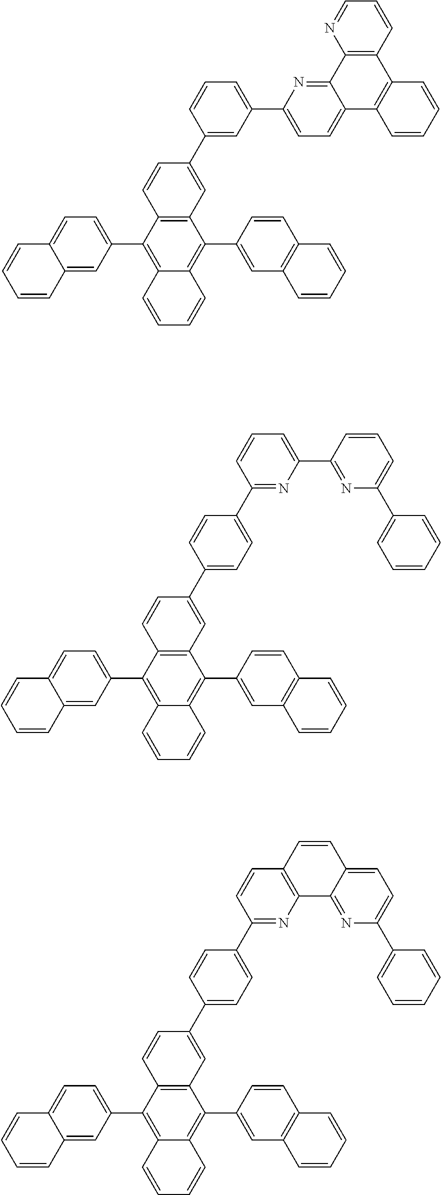 [SMILES: c1cc(-c2ccc3c(-c4ccc5ccccc5c4)c4ccccc4c(-c4ccc5ccccc5c4)c3c2)cc(-c2ccc3c4ccccc4c4cccnc4c3n2)c1 ; c1ccc(-c2cccc(-c3cccc(-c4ccc(-c5ccc6c(-c7ccc8ccccc8c7)c7ccccc7c(-c7ccc8ccccc8c7)c6c5)cc4)n3)n2)cc1 ; c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1]

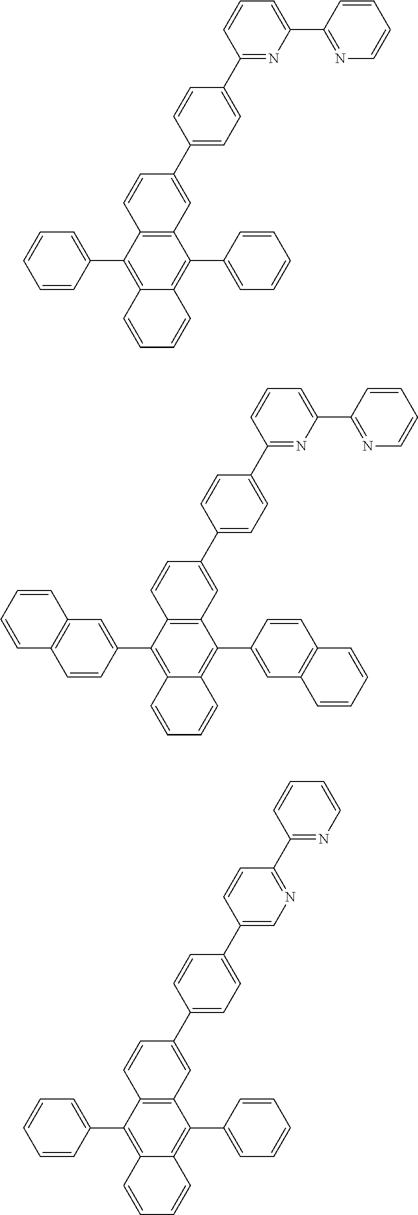 [SMILES: c1ccc(-c2c3ccccc3c(-c3ccccc3)c3cc(-c4ccc(-c5cccc(-c6ccccn6)n5)cc4)ccc23)cc1 ; c1ccc(-c2cccc(-c3ccc(-c4ccc5c(-c6ccc7ccccc7c6)c6ccccc6c(-c6ccc7ccccc7c6)c5c4)cc3)n2)nc1 ; c1ccc(-c2c3ccccc3c(-c3ccccc3)c3cc(-c4ccc(-c5ccc(-c6ccccn6)nc5)cc4)ccc23)cc1]

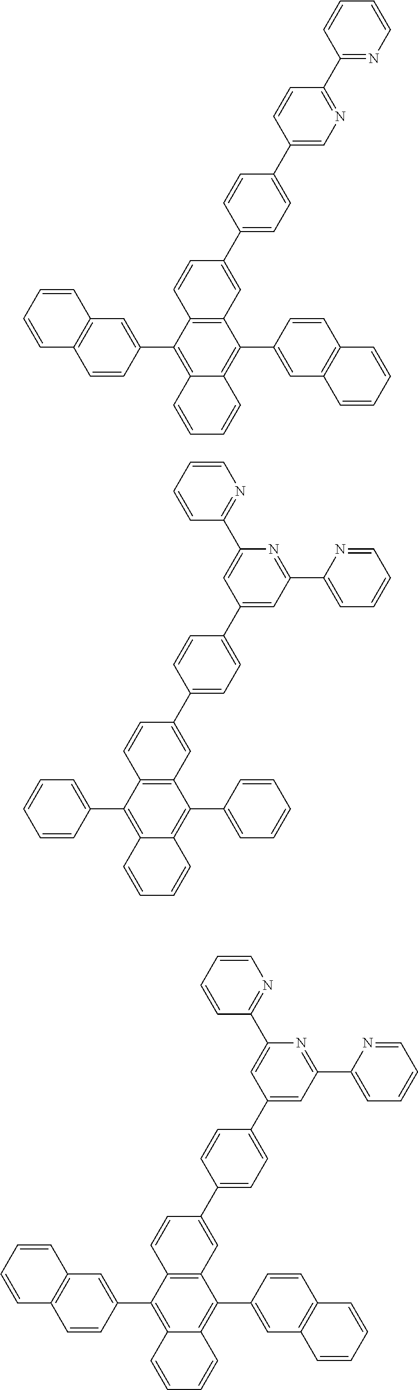

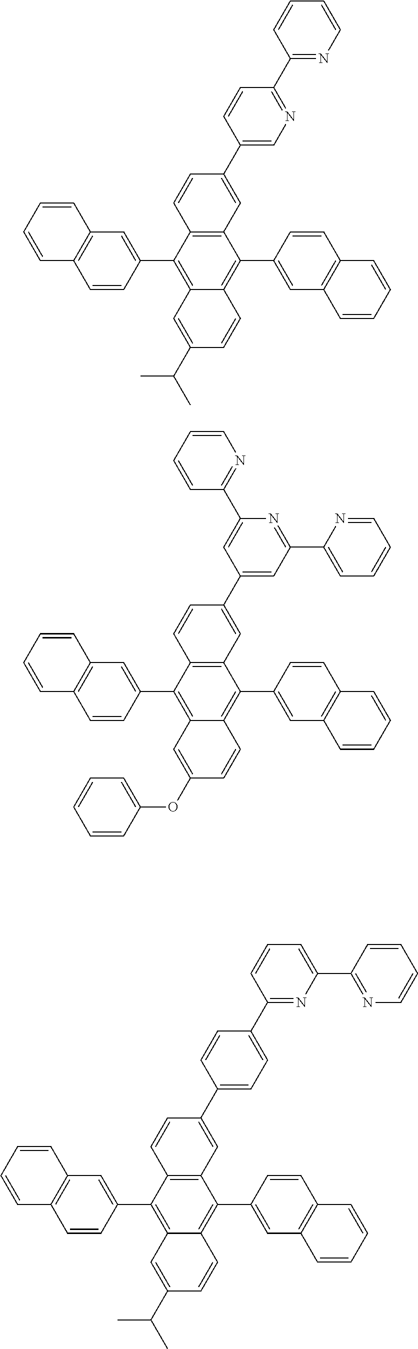

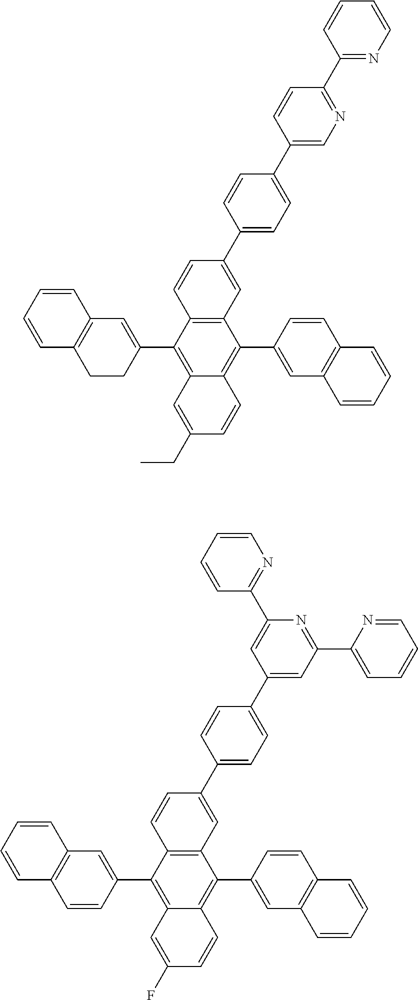 [SMILES: CCc1ccc2c(-c3ccc4ccccc4c3)c3cc(-c4ccc(-c5ccc(-c6ccccn6)nc5)cc4)ccc3c(C3=Cc4ccccc4CC3)c2c1 ; Fc1ccc2c(-c3ccc4ccccc4c3)c3cc(-c4ccc(-c5cc(-c6ccccn6)nc(-c6ccccn6)c5)cc4)ccc3c(-c3ccc4ccccc4c3)c2c1]

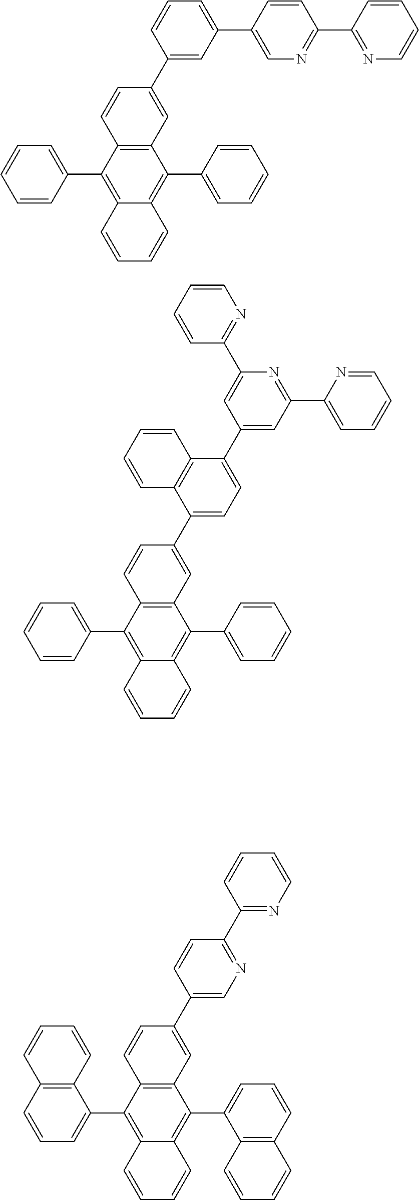

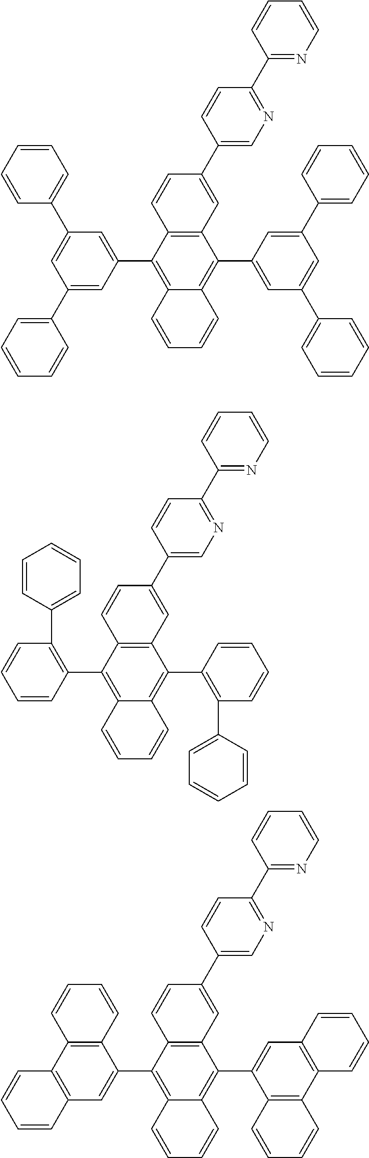

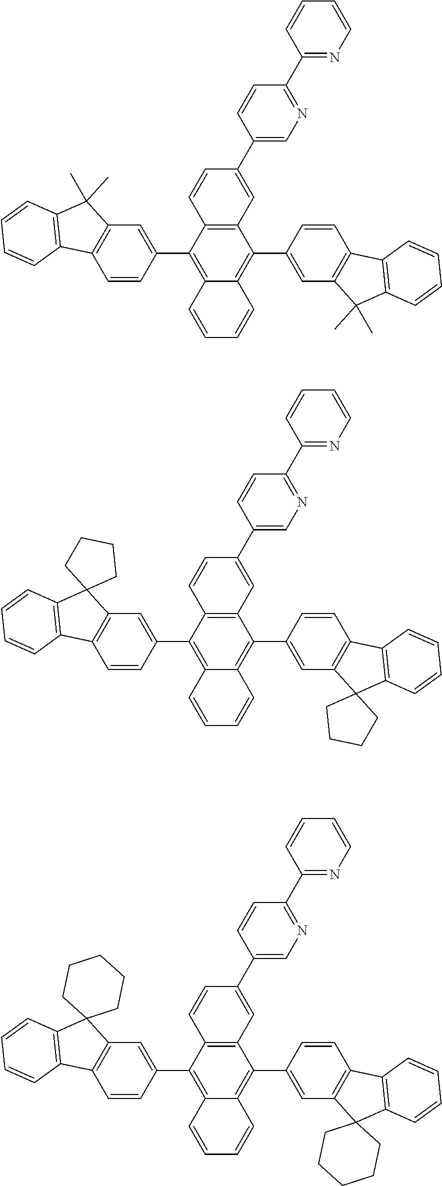 [SMILES: CC1(C)c2ccccc2-c2ccc(-c3c4ccccc4c(-c4ccc5c(c4)C(C)(C)c4ccccc4-5)c4cc(-c5ccc(-c6ccccn6)nc5)ccc34)cc21 ; c1ccc(-c2ccc(-c3ccc4c(-c5ccc6c(c5)C5(CCCC5)c5ccccc5-6)c5ccccc5c(-c5ccc6c(c5)C5(CCCC5)c5ccccc5-6)c4c3)cn2)nc1 ; c1ccc(-c2ccc(-c3ccc4c(-c5ccc6c(c5)C5(CCCCC5)c5ccccc5-6)c5ccccc5c(-c5ccc6c(c5)C5(CCCCC5)c5ccccc5-6)c4c3)cn2)nc1]

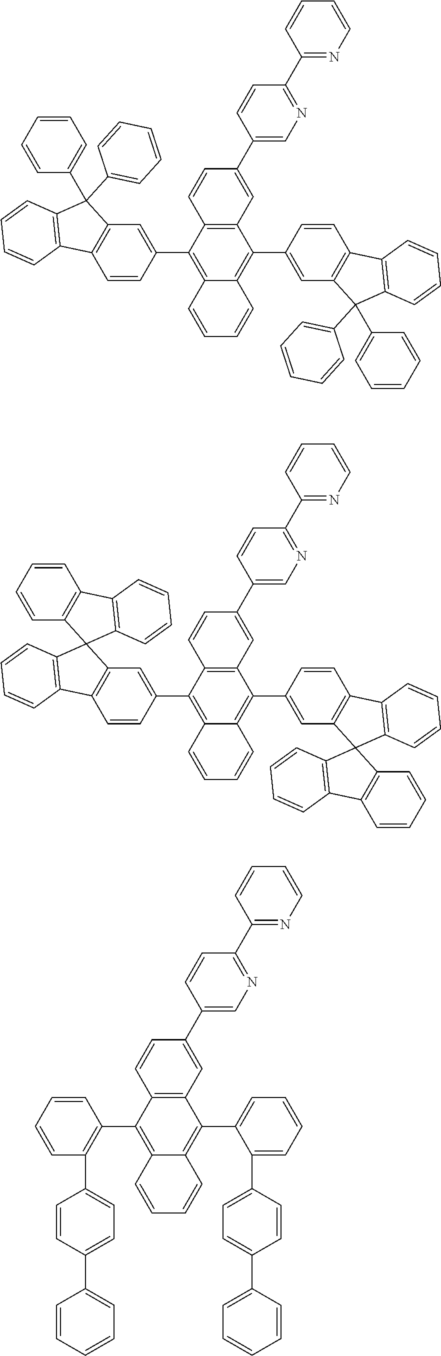 [SMILES: c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccc(-c7ccccn7)nc6)ccc45)cc32)cc1 ; c1ccc(-c2ccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)cn2)nc1 ; c1ccc(-c2ccc(-c3ccccc3-c3c4ccccc4c(-c4ccccc4-c4ccc(-c5ccccc5)cc4)c4cc(-c5ccc(-c6ccccn6)nc5)ccc34)cc2)cc1]

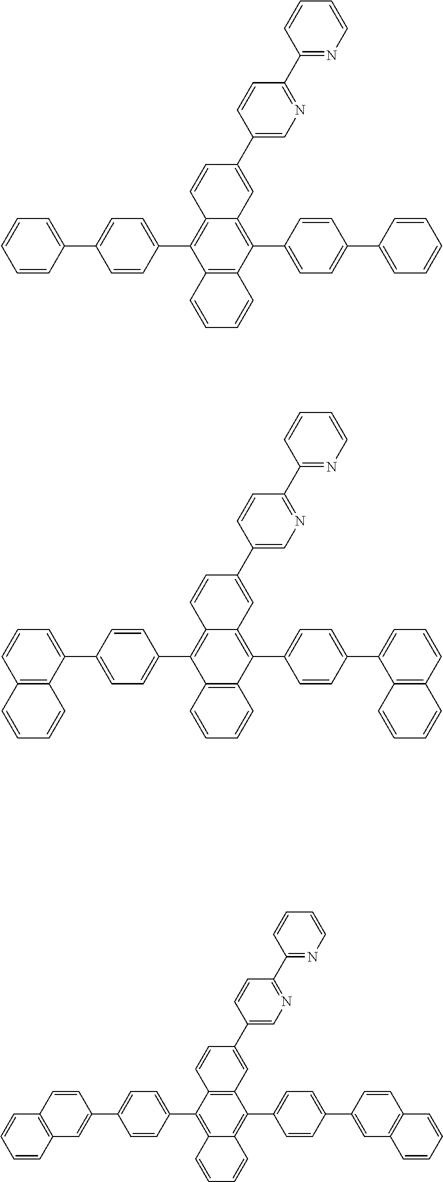 [SMILES: c1ccc(-c2ccc(-c3c4ccccc4c(-c4ccc(-c5ccccc5)cc4)c4cc(-c5ccc(-c6ccccn6)nc5)ccc34)cc2)cc1 ; c1ccc(-c2ccc(-c3ccc4c(-c5ccc(-c6cccc7ccccc67)cc5)c5ccccc5c(-c5ccc(-c6cccc7ccccc67)cc5)c4c3)cn2)nc1 ; c1ccc(-c2ccc(-c3ccc4c(-c5ccc(-c6ccc7ccccc7c6)cc5)c5ccccc5c(-c5ccc(-c6ccc7ccccc7c6)cc5)c4c3)cn2)nc1]

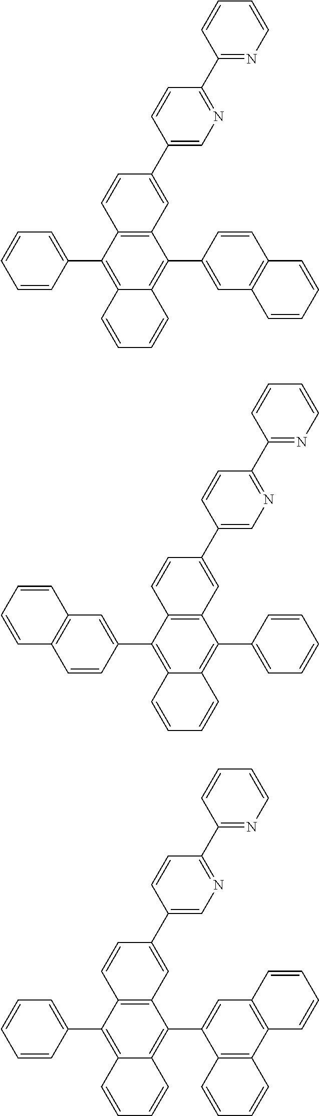

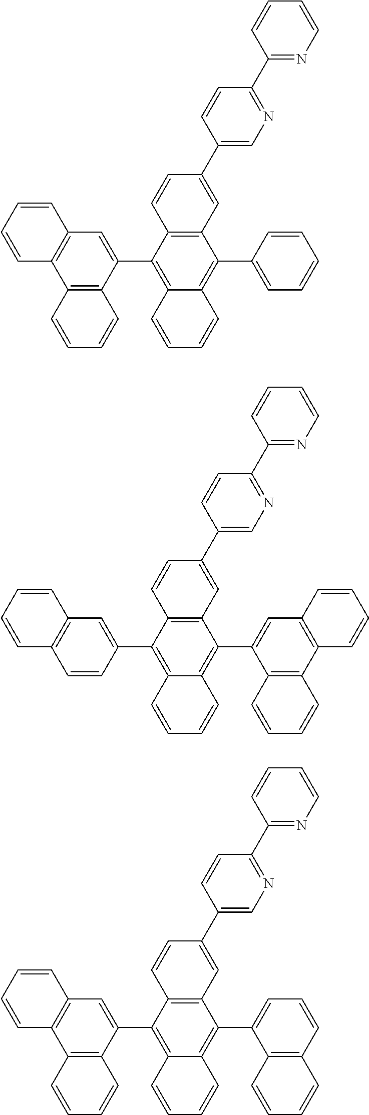

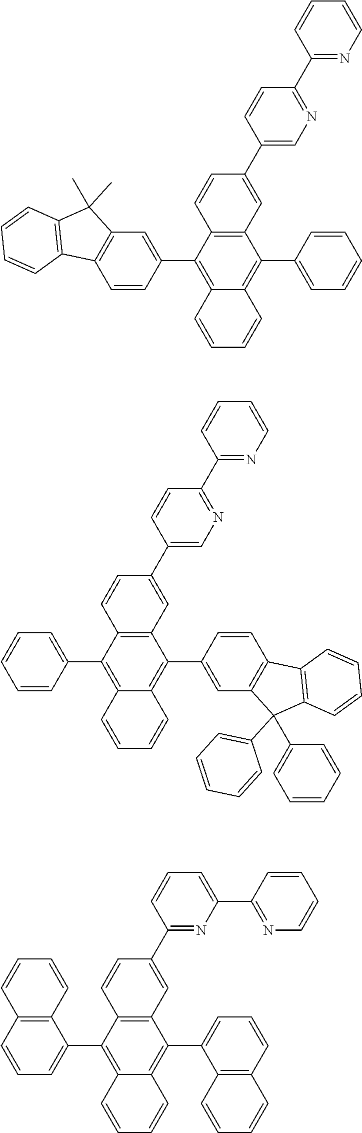 [SMILES: CC1(C)c2ccccc2-c2ccc(-c3c4ccccc4c(-c4ccccc4)c4cc(-c5ccc(-c6ccccn6)nc5)ccc34)cc21 ; c1ccc(-c2c3ccccc3c(-c3ccc4c(c3)C(c3ccccc3)(c3ccccc3)c3ccccc3-4)c3cc(-c4ccc(-c5ccccn5)nc4)ccc23)cc1 ; c1ccc(-c2cccc(-c3ccc4c(-c5cccc6ccccc56)c5ccccc5c(-c5cccc6ccccc56)c4c3)n2)nc1]

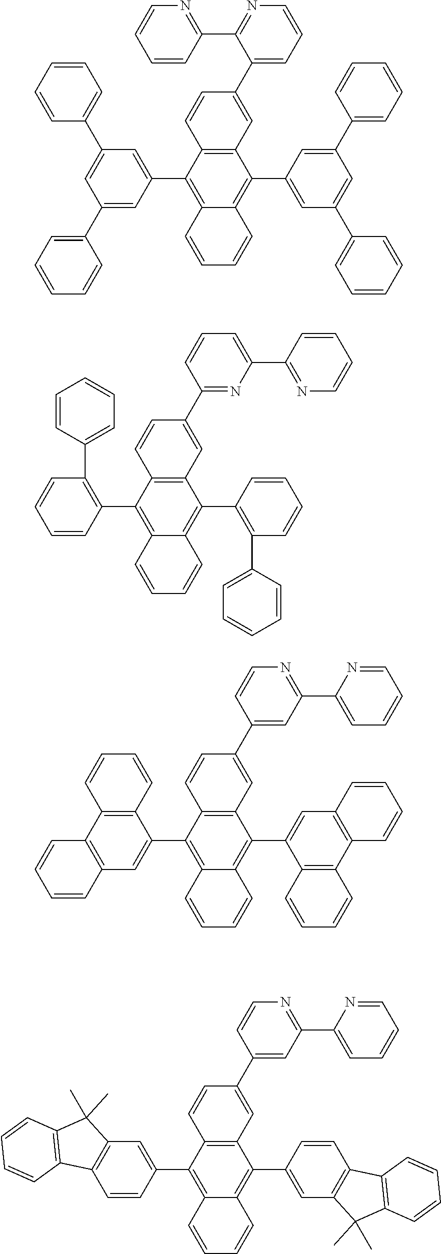

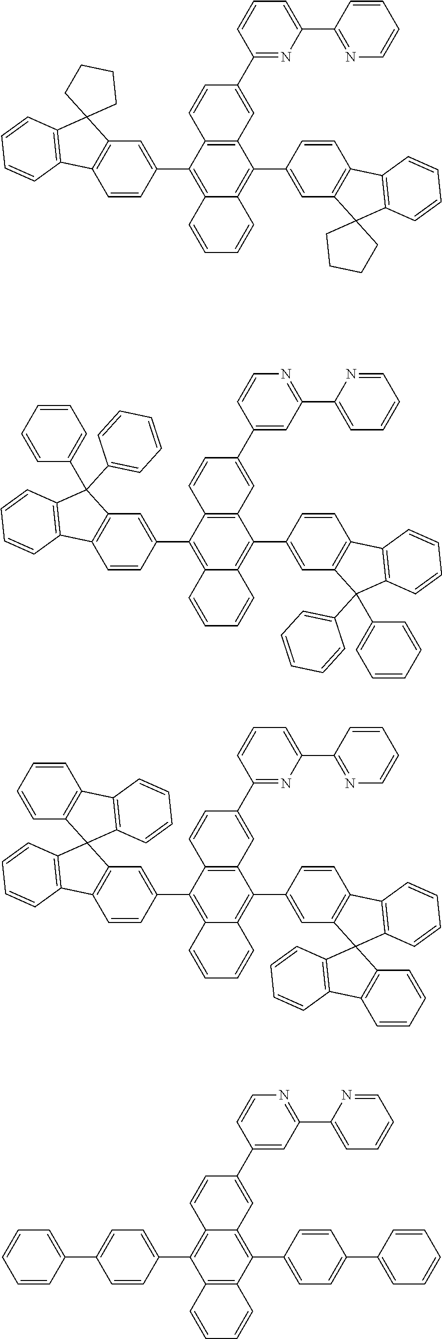 [SMILES: c1ccc(-c2cccc(-c3ccc4c(-c5ccc6c(c5)C5(CCCC5)c5ccccc5-6)c5ccccc5c(-c5ccc6c(c5)C5(CCCC5)c5ccccc5-6)c4c3)n2)nc1 ; c1ccc(C2(c3ccccc3)c3ccccc3-c3ccc(-c4c5ccccc5c(-c5ccc6c(c5)C(c5ccccc5)(c5ccccc5)c5ccccc5-6)c5cc(-c6ccnc(-c7ccccn7)c6)ccc45)cc32)cc1 ; c1ccc(-c2cccc(-c3ccc4c(-c5ccc6c(c5)C5(c7ccccc7-c7ccccc75)c5ccccc5-6)c5ccccc5c(-c5ccc6c(c5)C5(c7ccccc7-c7ccccc75)c5ccccc5-6)c4c3)n2)nc1 ; c1ccc(-c2ccc(-c3c4ccccc4c(-c4ccc(-c5ccccc5)cc4)c4cc(-c5ccnc(-c6ccccn6)c5)ccc34)cc2)cc1]

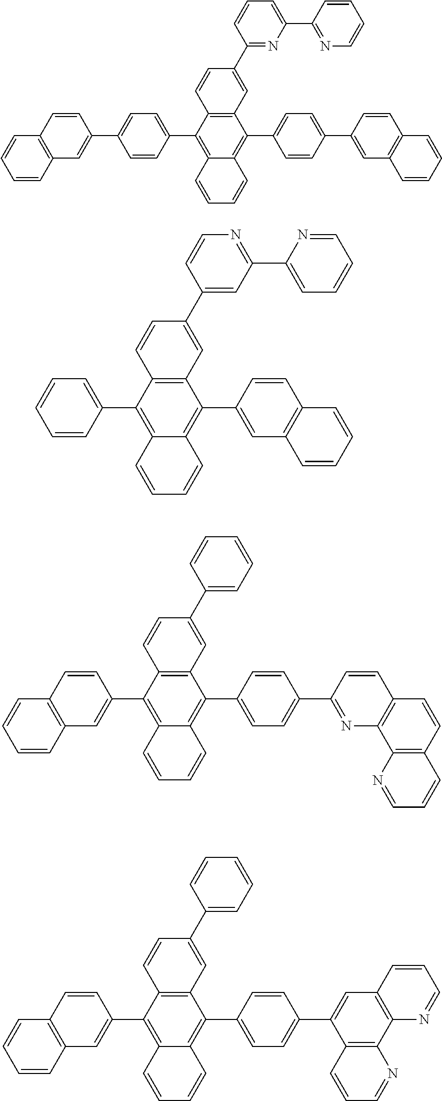

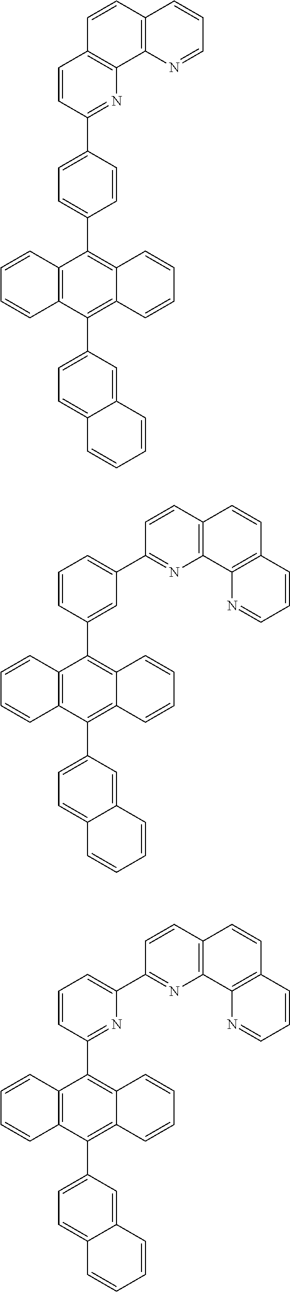 [SMILES: c1ccc2cc(-c3c4ccccc4c(-c4ccc(-c5ccc6ccc7cccnc7c6n5)cc4)c4ccccc34)ccc2c1 ; c1cc(-c2ccc3ccc4cccnc4c3n2)cc(-c2c3ccccc3c(-c3ccc4ccccc4c3)c3ccccc23)c1 ; c1cc(-c2ccc3ccc4cccnc4c3n2)nc(-c2c3ccccc3c(-c3ccc4ccccc4c3)c3ccccc23)c1]

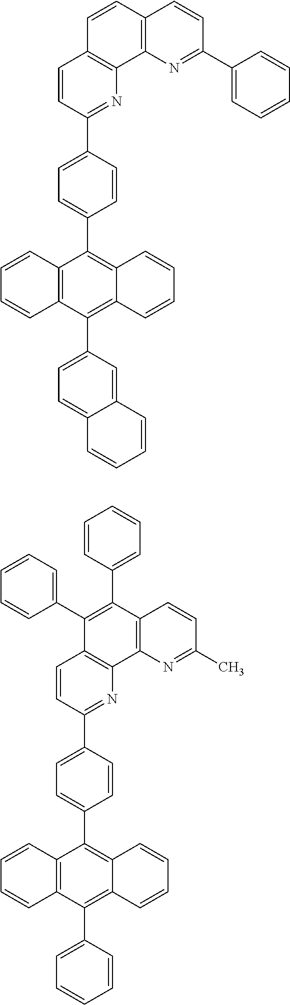

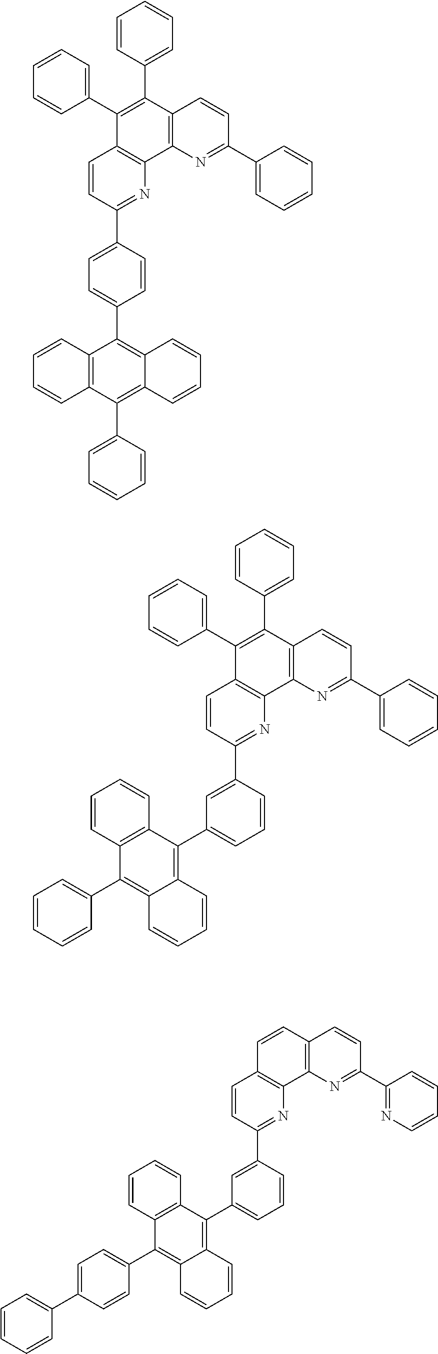 [SMILES: c1ccc(-c2ccc3c(-c4ccccc4)c(-c4ccccc4)c4ccc(-c5ccc(-c6c7ccccc7c(-c7ccccc7)c7ccccc67)cc5)nc4c3n2)cc1 ; c1ccc(-c2ccc3c(-c4ccccc4)c(-c4ccccc4)c4ccc(-c5cccc(-c6c7ccccc7c(-c7ccccc7)c7ccccc67)c5)nc4c3n2)cc1 ; c1ccc(-c2ccc(-c3c4ccccc4c(-c4cccc(-c5ccc6ccc7ccc(-c8ccccn8)nc7c6n5)c4)c4ccccc34)cc2)cc1]

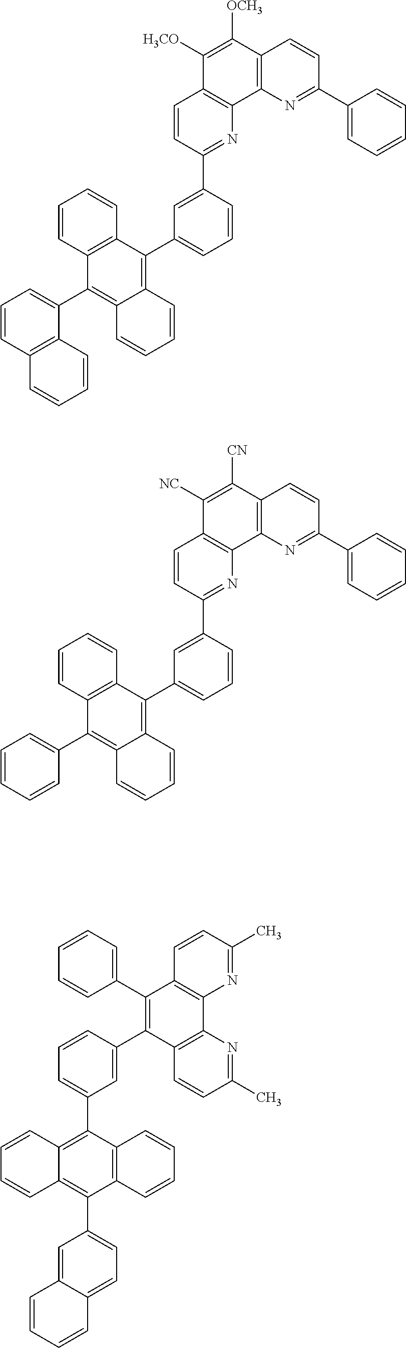 [SMILES: COc1c(OC)c2ccc(-c3cccc(-c4c5ccccc5c(-c5cccc6ccccc56)c5ccccc45)c3)nc2c2nc(-c3ccccc3)ccc12 ; N#Cc1c(C#N)c2ccc(-c3cccc(-c4c5ccccc5c(-c5ccccc5)c5ccccc45)c3)nc2c2nc(-c3ccccc3)ccc12 ; Cc1ccc2c(-c3ccccc3)c(-c3cccc(-c4c5ccccc5c(-c5ccc6ccccc6c5)c5ccccc45)c3)c3ccc(C)nc3c2n1]

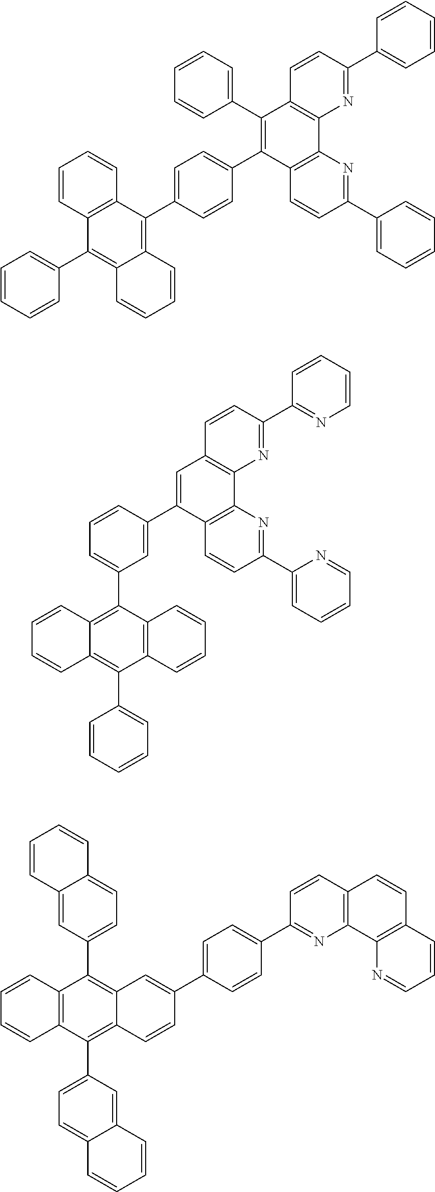 [SMILES: c1ccc(-c2ccc3c(-c4ccccc4)c(-c4ccc(-c5c6ccccc6c(-c6ccccc6)c6ccccc56)cc4)c4ccc(-c5ccccc5)nc4c3n2)cc1 ; c1ccc(-c2c3ccccc3c(-c3cccc(-c4cc5ccc(-c6ccccn6)nc5c5nc(-c6ccccn6)ccc45)c3)c3ccccc23)cc1 ; c1ccc2cc(-c3c4ccccc4c(-c4ccc5ccccc5c4)c4cc(-c5ccc(-c6ccc7ccc8cccnc8c7n6)cc5)ccc34)ccc2c1]

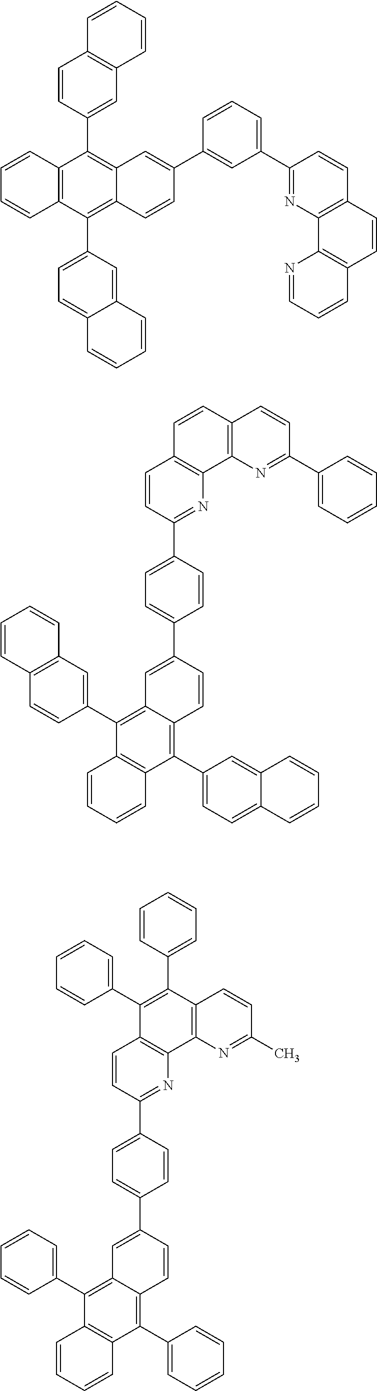 [SMILES: c1cc(-c2ccc3c(-c4ccc5ccccc5c4)c4ccccc4c(-c4ccc5ccccc5c4)c3c2)cc(-c2ccc3ccc4cccnc4c3n2)c1 ; c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1 ; Cc1ccc2c(-c3ccccc3)c(-c3ccccc3)c3ccc(-c4ccc(-c5ccc6c(-c7ccccc7)c7ccccc7c(-c7ccccc7)c6c5)cc4)nc3c2n1]

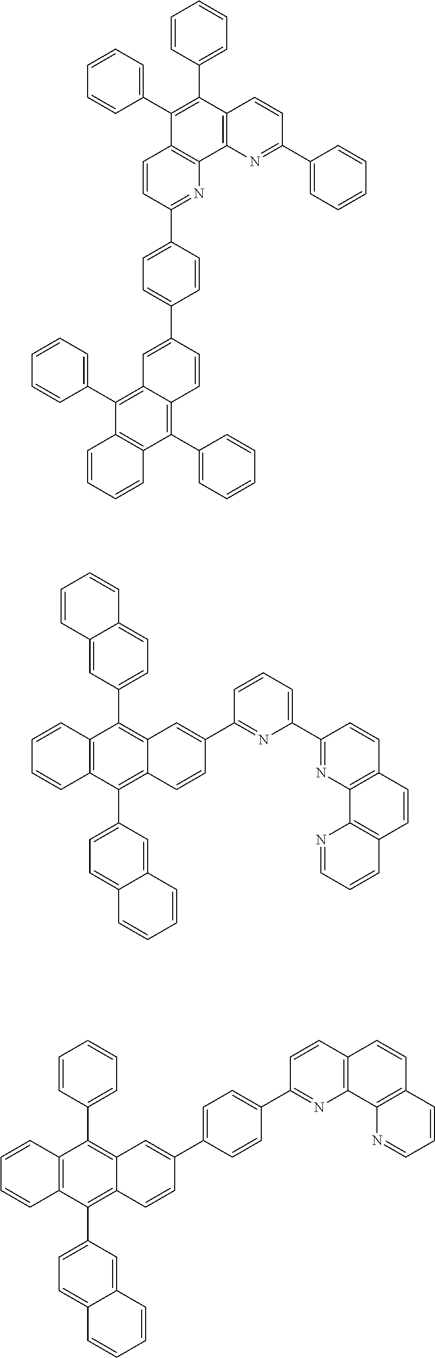 [SMILES: c1ccc(-c2ccc3c(-c4ccccc4)c(-c4ccccc4)c4ccc(-c5ccc(-c6ccc7c(-c8ccccc8)c8ccccc8c(-c8ccccc8)c7c6)cc5)nc4c3n2)cc1 ; c1cc(-c2ccc3c(-c4ccc5ccccc5c4)c4ccccc4c(-c4ccc5ccccc5c4)c3c2)nc(-c2ccc3ccc4cccnc4c3n2)c1 ; c1ccc(-c2c3ccccc3c(-c3ccc4ccccc4c3)c3ccc(-c4ccc(-c5ccc6ccc7cccnc7c6n5)cc4)cc23)cc1]

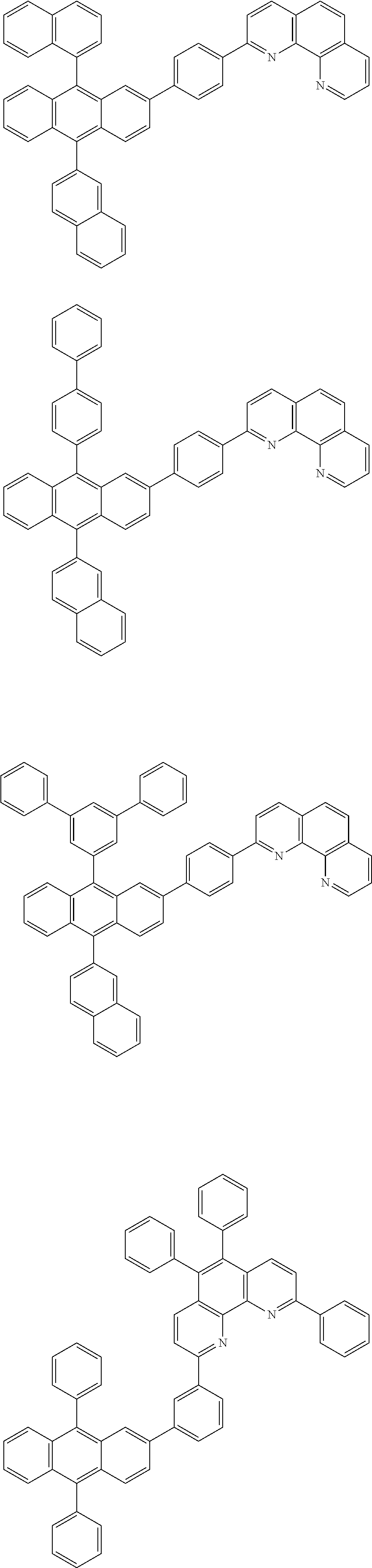 [SMILES: c1ccc2cc(-c3c4ccccc4c(-c4cccc5ccccc45)c4cc(-c5ccc(-c6ccc7ccc8cccnc8c7n6)cc5)ccc34)ccc2c1 ; c1ccc(-c2ccc(-c3c4ccccc4c(-c4ccc5ccccc5c4)c4ccc(-c5ccc(-c6ccc7ccc8cccnc8c7n6)cc5)cc34)cc2)cc1 ; c1ccc(-c2cc(-c3ccccc3)cc(-c3c4ccccc4c(-c4ccc5ccccc5c4)c4ccc(-c5ccc(-c6ccc7ccc8cccnc8c7n6)cc5)cc34)c2)cc1 ; c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1]

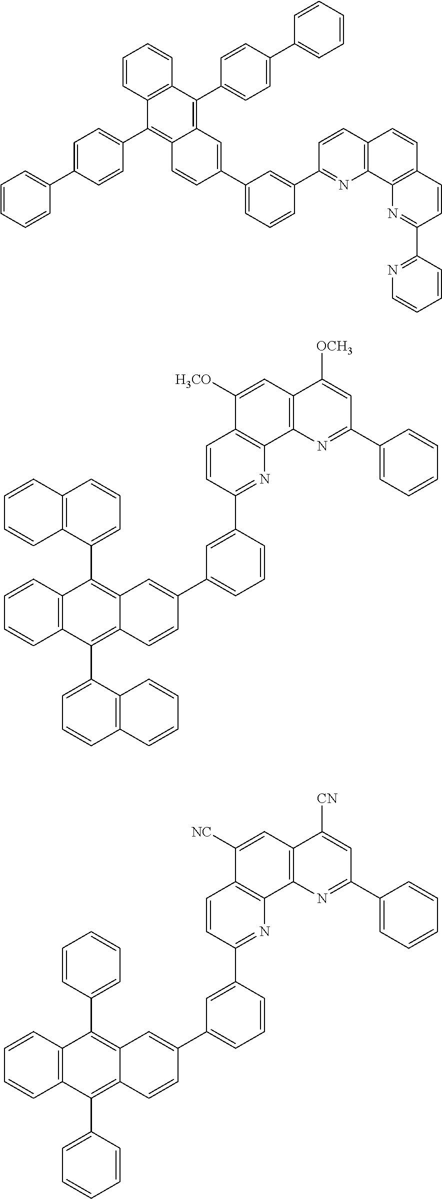 [SMILES: c1ccc(-c2ccc(-c3c4ccccc4c(-c4ccc(-c5ccccc5)cc4)c4cc(-c5cccc(-c6ccc7ccc8ccc(-c9ccccn9)nc8c7n6)c5)ccc34)cc2)cc1 ; COc1cc2c(OC)cc(-c3ccccc3)nc2c2nc(-c3cccc(-c4ccc5c(-c6cccc7ccccc67)c6ccccc6c(-c6cccc7ccccc67)c5c4)c3)ccc12 ; N#Cc1cc2c(C#N)cc(-c3ccccc3)nc2c2nc(-c3cccc(-c4ccc5c(-c6ccccc6)c6ccccc6c(-c6ccccc6)c5c4)c3)ccc12]

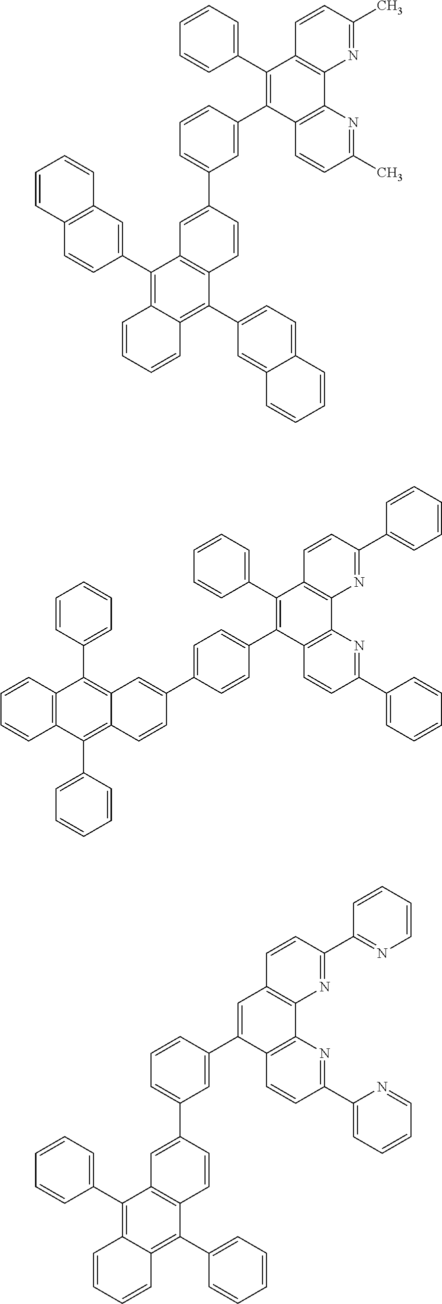 [SMILES: Cc1ccc2c(-c3ccccc3)c(-c3cccc(-c4ccc5c(-c6ccc7ccccc7c6)c6ccccc6c(-c6ccc7ccccc7c6)c5c4)c3)c3ccc(C)nc3c2n1 ; c1ccc(-c2ccc3c(-c4ccccc4)c(-c4ccc(-c5ccc6c(-c7ccccc7)c7ccccc7c(-c7ccccc7)c6c5)cc4)c4ccc(-c5ccccc5)nc4c3n2)cc1 ; c1ccc(-c2c3ccccc3c(-c3ccccc3)c3cc(-c4cccc(-c5cc6ccc(-c7ccccn7)nc6c6nc(-c7ccccn7)ccc56)c4)ccc23)cc1]

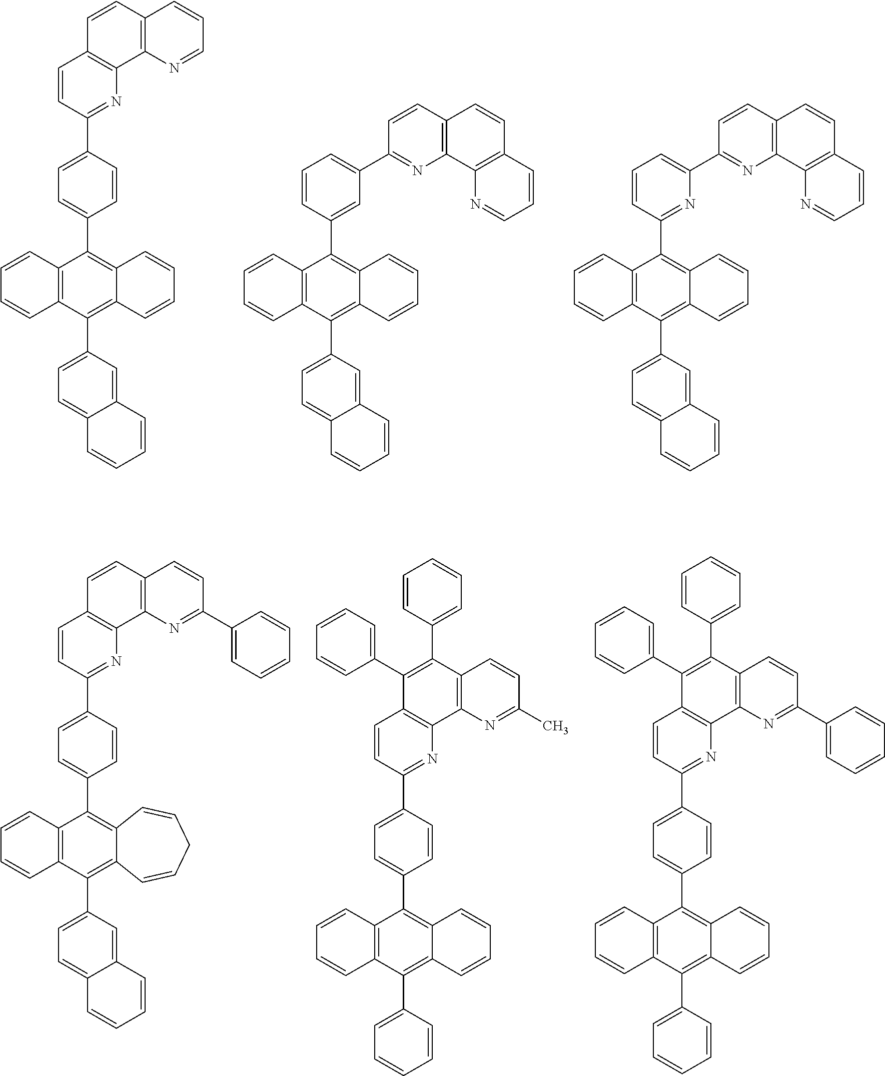

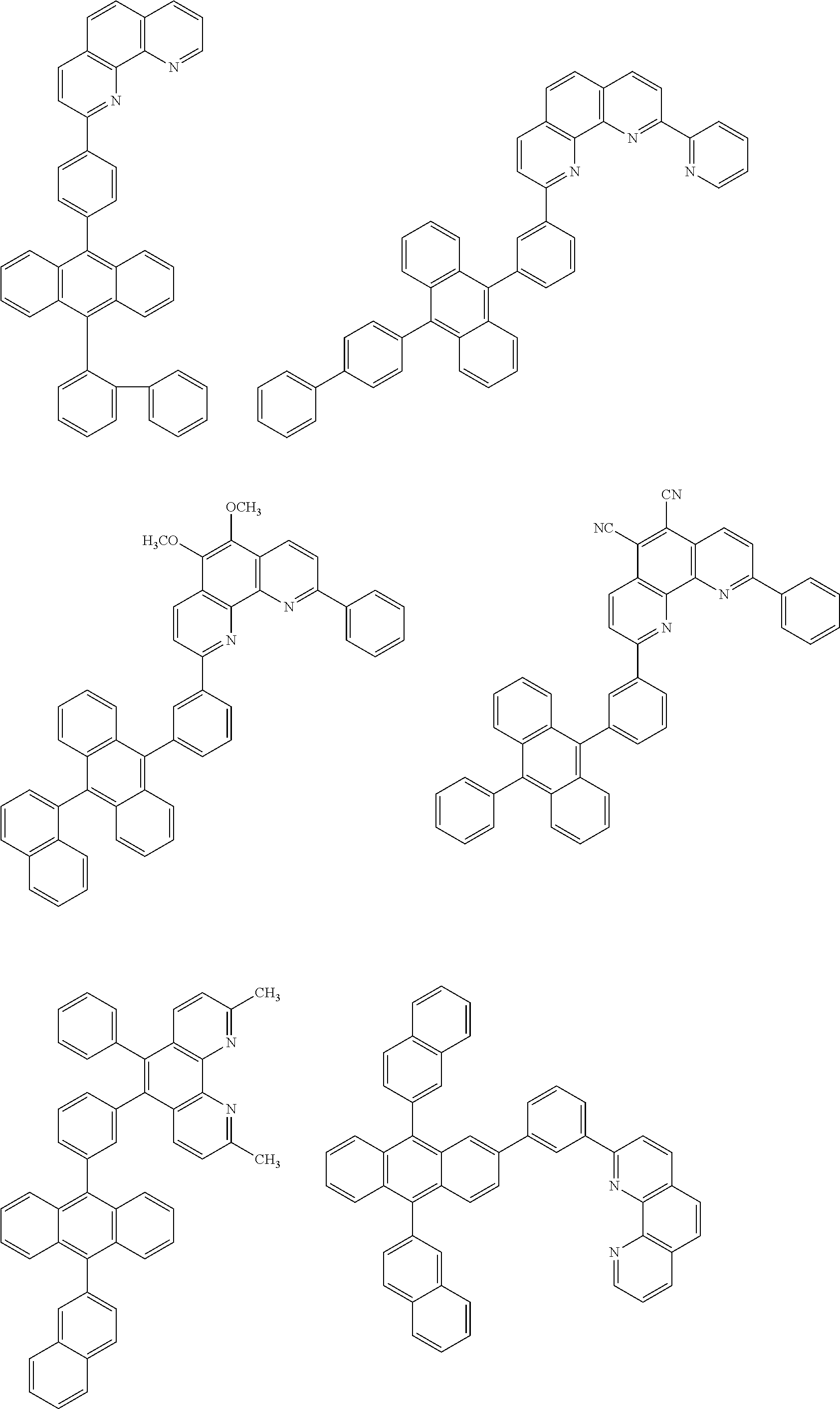

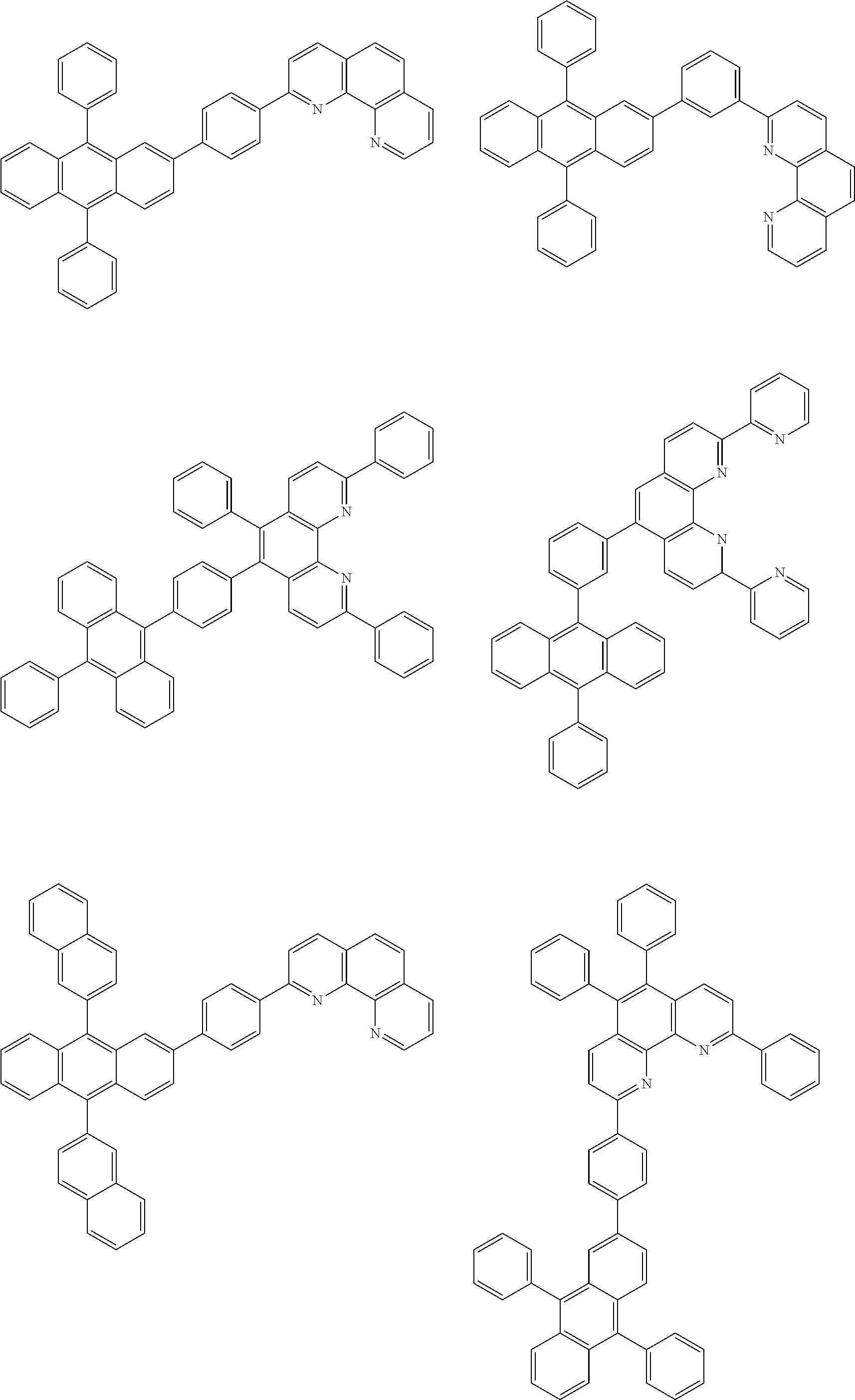

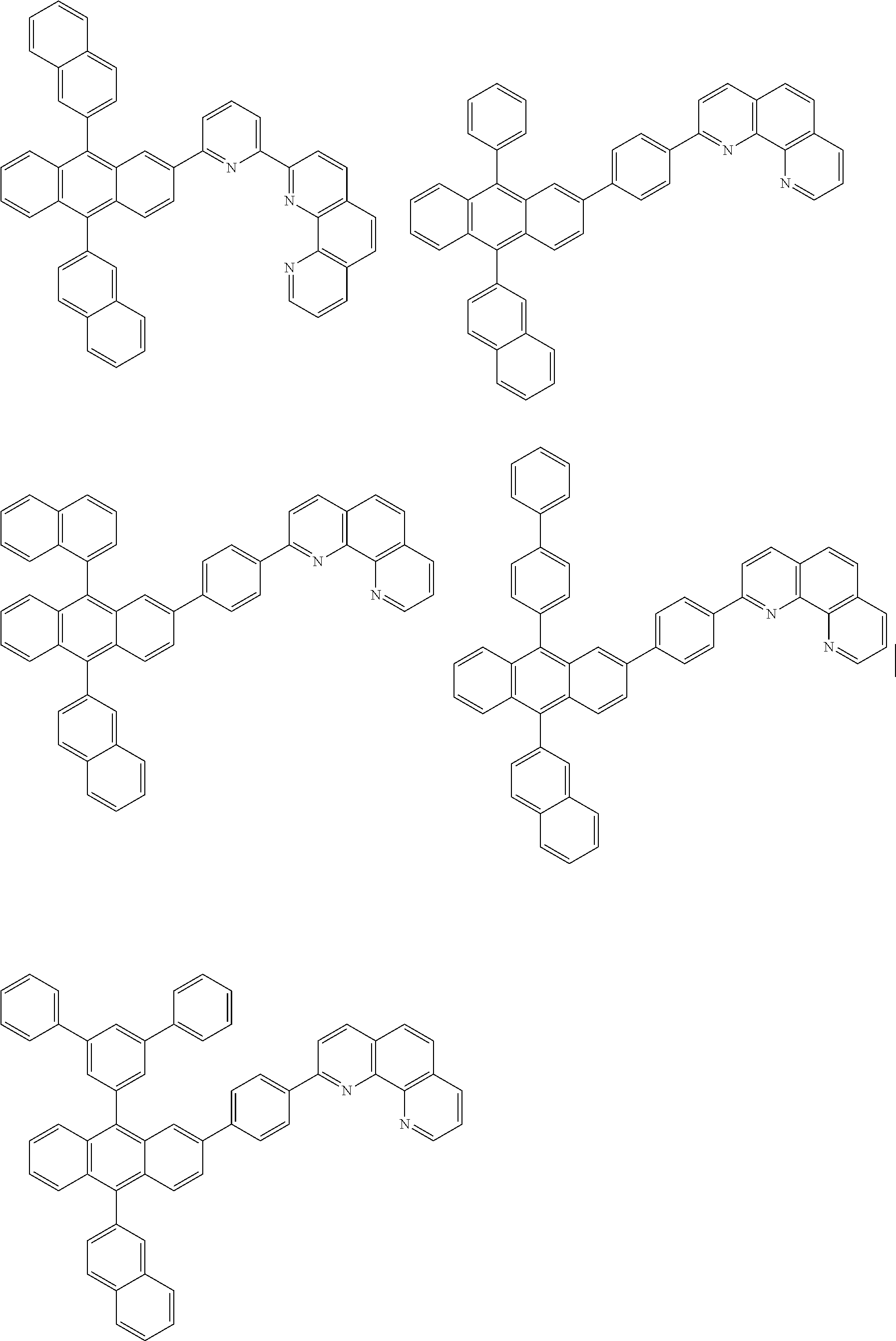

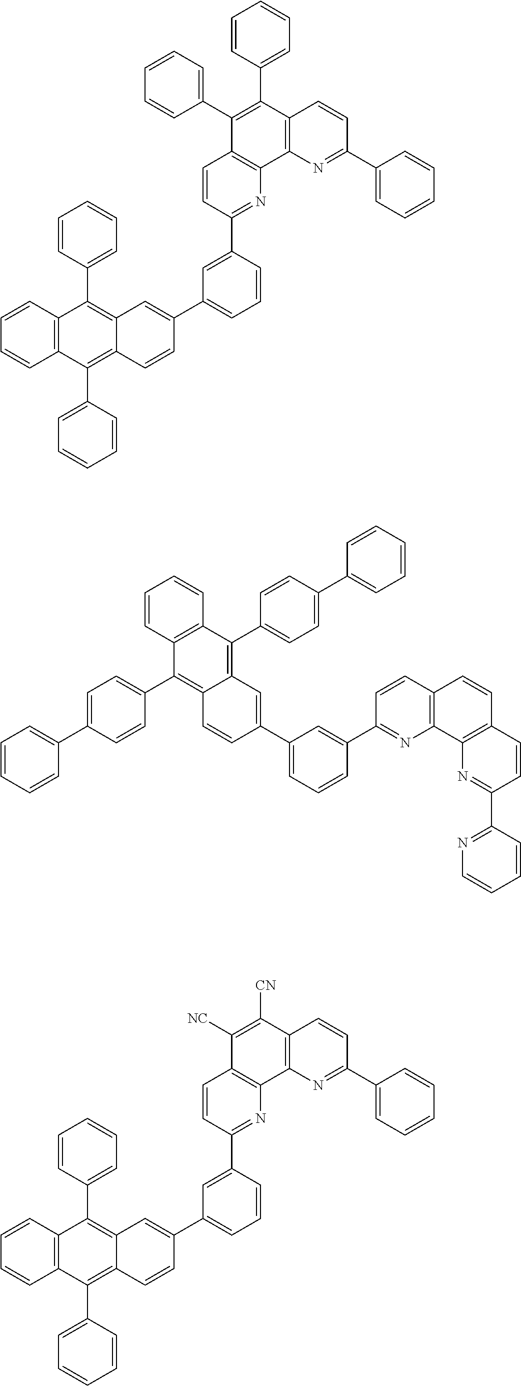 [SMILES: c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1 ; c1ccc(-c2ccc(-c3c4ccccc4c(-c4ccc(-c5ccccc5)cc4)c4cc(-c5cccc(-c6ccc7ccc8ccc(-c9ccccn9)nc8c7n6)c5)ccc34)cc2)cc1 ; N#Cc1c(C#N)c2ccc(-c3cccc(-c4ccc5c(-c6ccccc6)c6ccccc6c(-c6ccccc6)c5c4)c3)nc2c2nc(-c3ccccc3)ccc12]

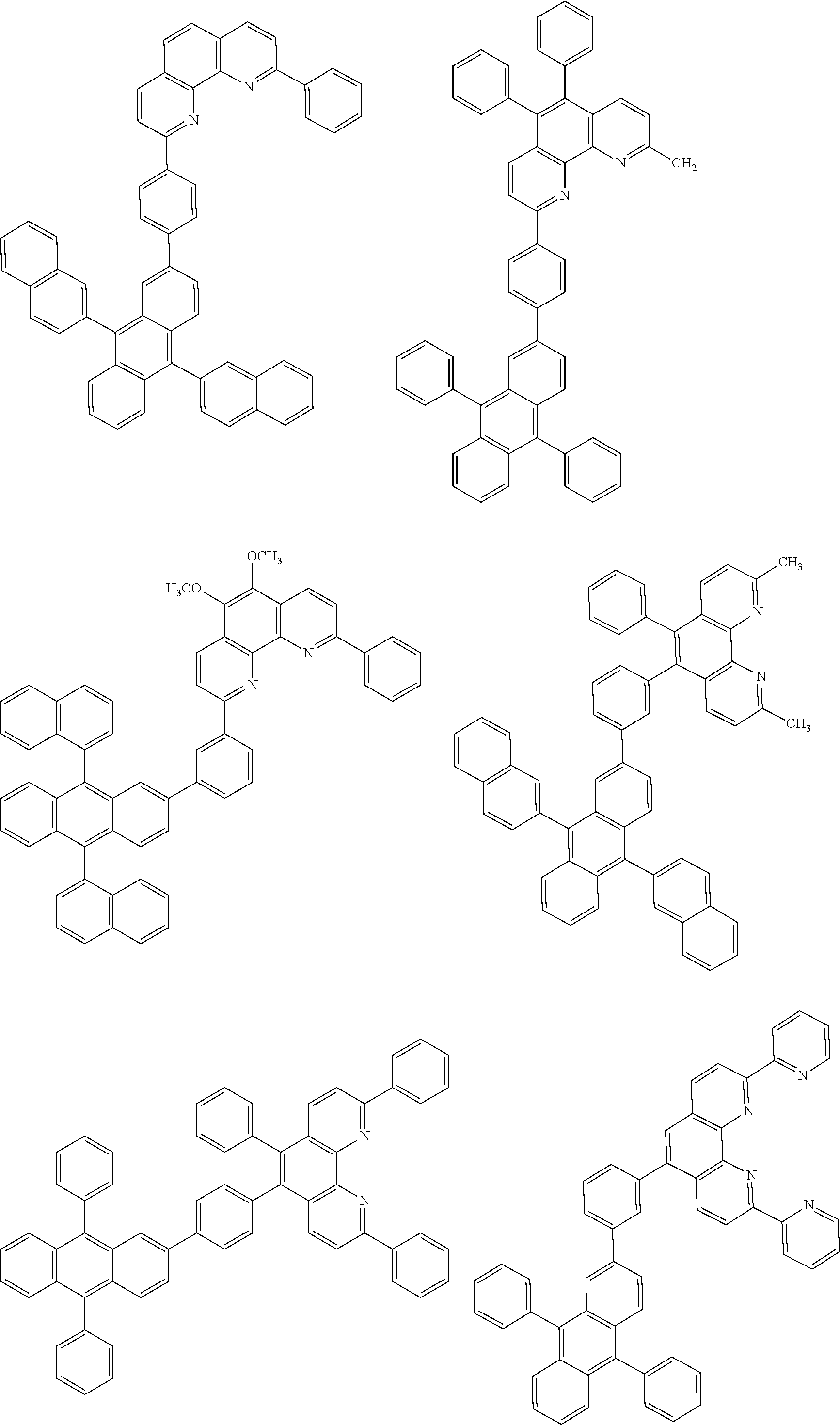

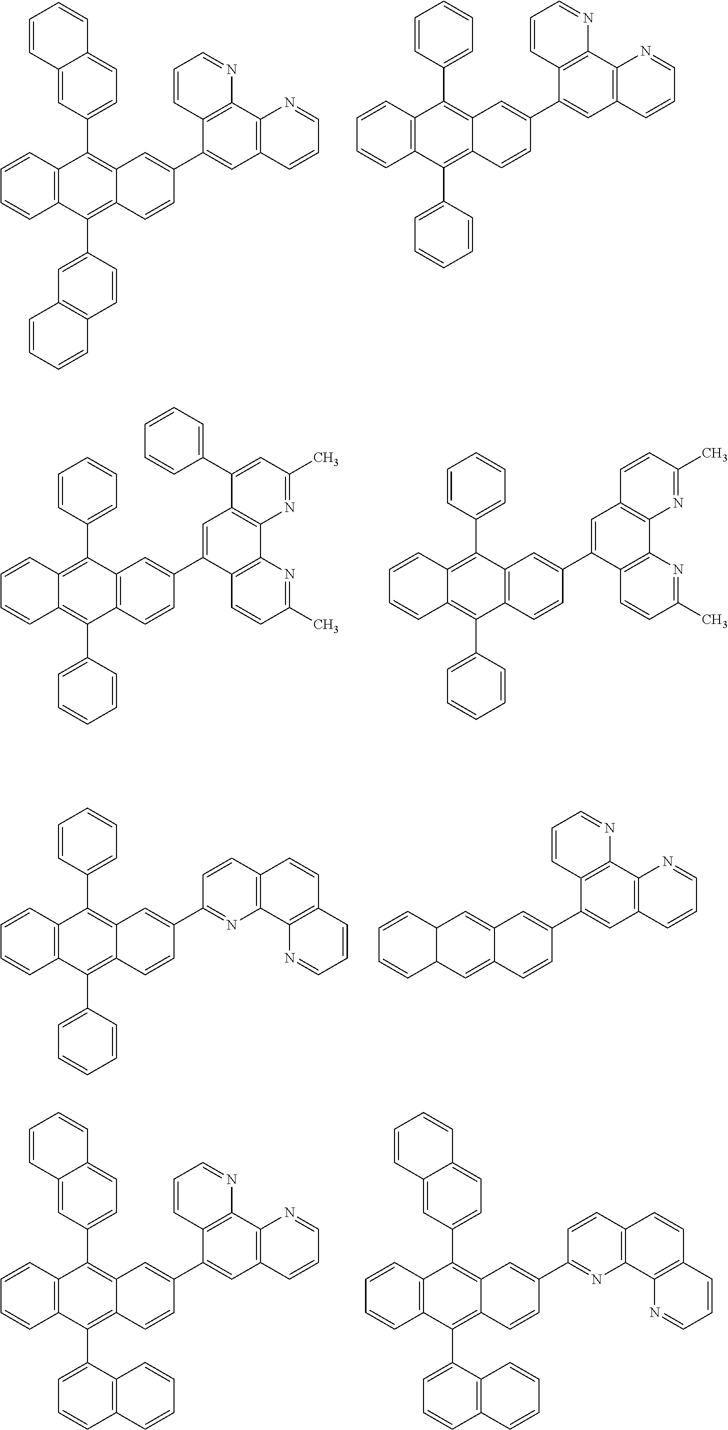

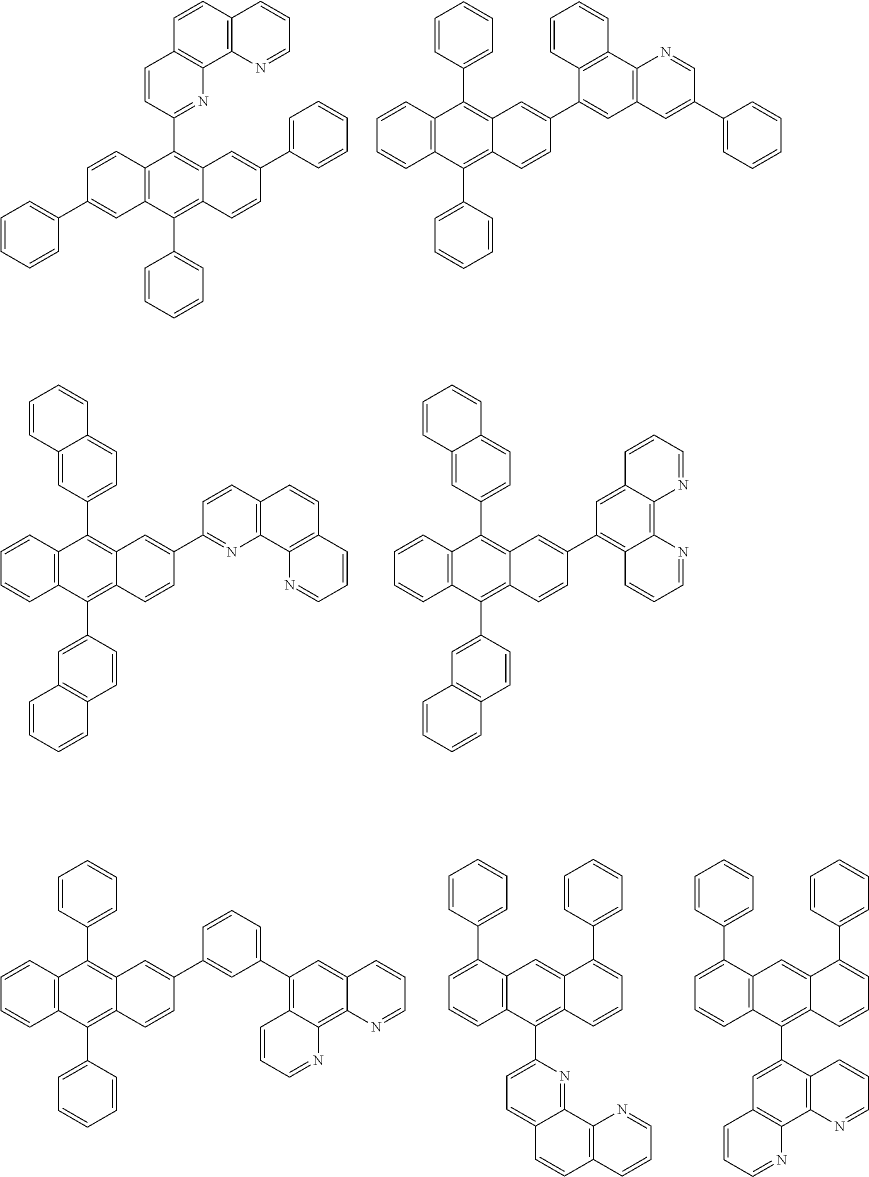

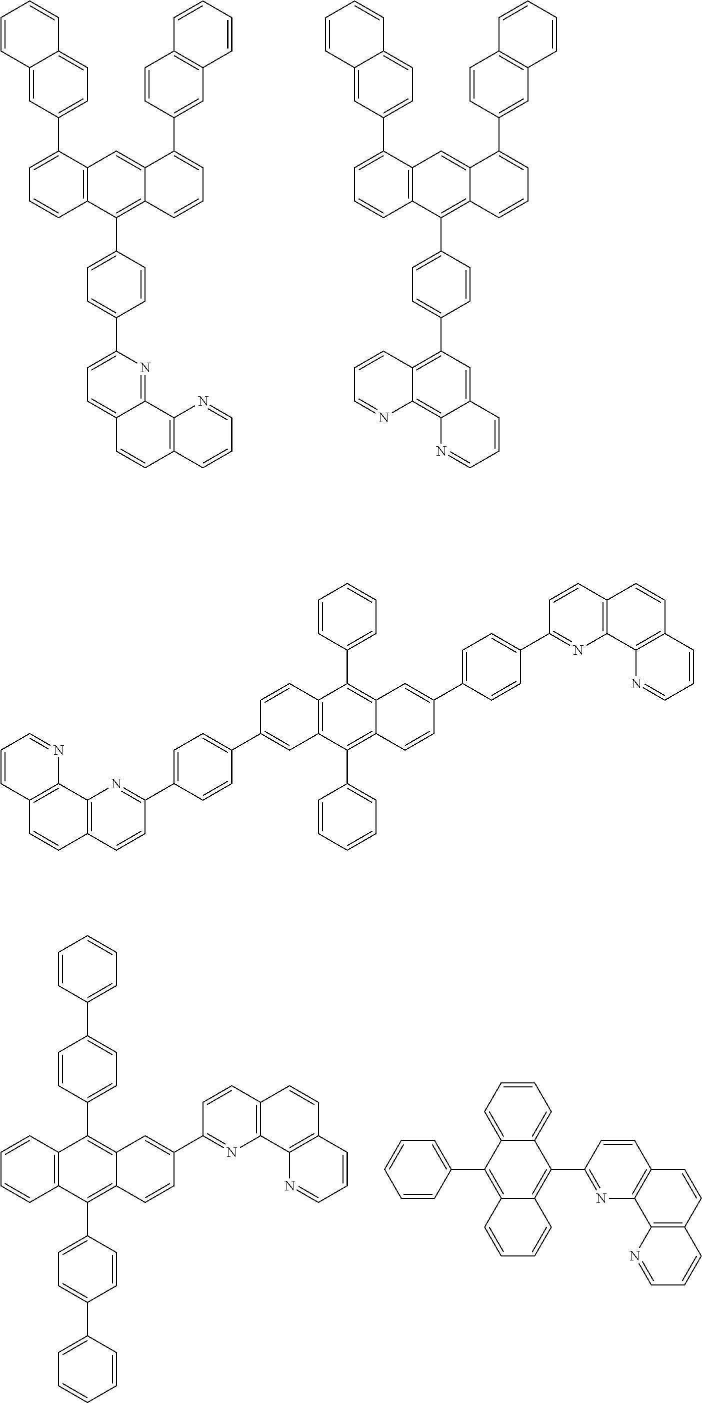

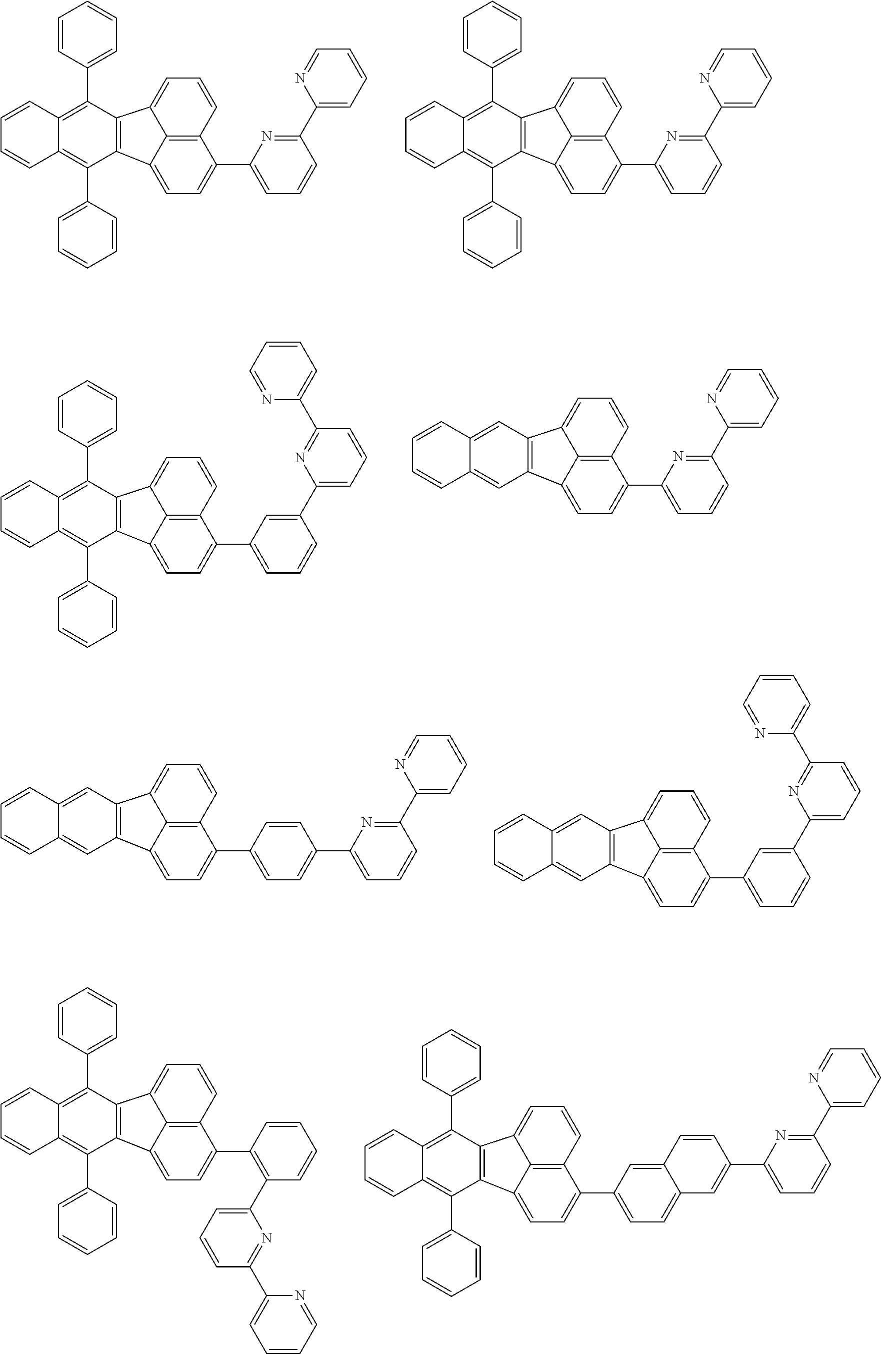

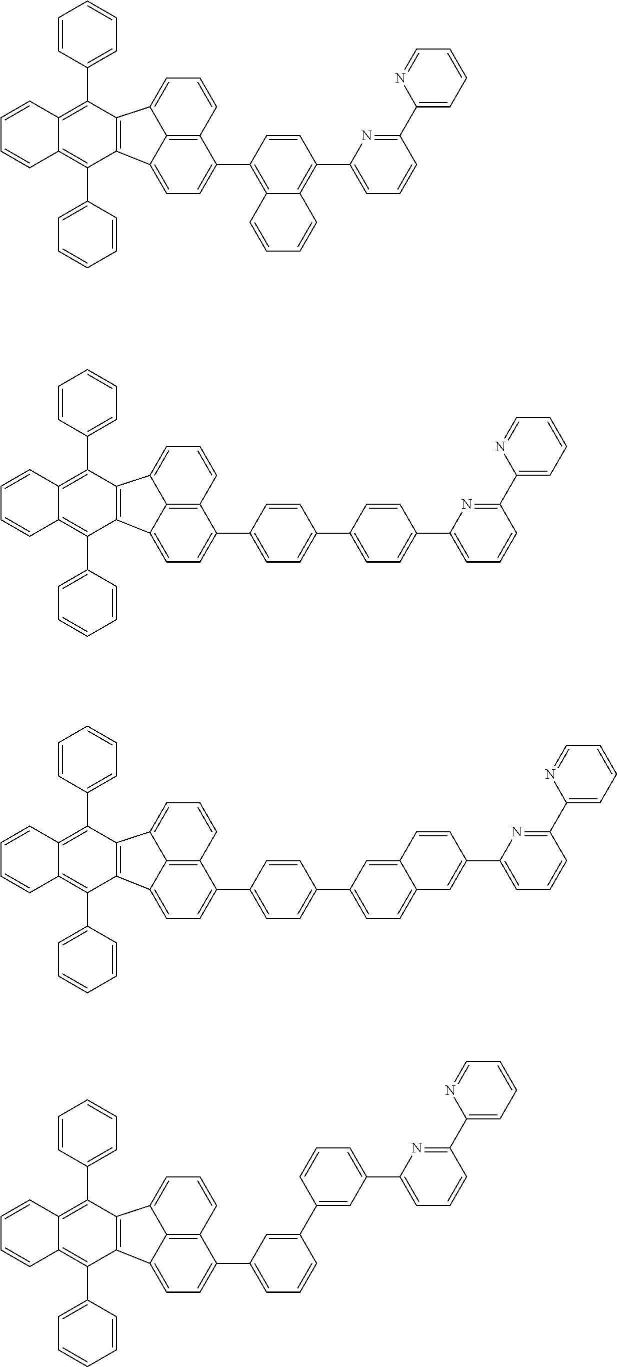 [SMILES: c1ccc(-c2c3c(c(-c4ccccc4)c4ccccc24)-c2ccc(-c4ccc(-c5cccc(-c6ccccn6)n5)c5ccccc45)c4cccc-3c24)cc1 ; c1ccc(-c2c3c(c(-c4ccccc4)c4ccccc24)-c2ccc(-c4ccc(-c5ccc(-c6cccc(-c7ccccn7)n6)cc5)cc4)c4cccc-3c24)cc1 ; c1ccc(-c2c3c(c(-c4ccccc4)c4ccccc24)-c2ccc(-c4ccc(-c5ccc6cc(-c7cccc(-c8ccccn8)n7)ccc6c5)cc4)c4cccc-3c24)cc1 ; c1ccc(-c2c3c(c(-c4ccccc4)c4ccccc24)-c2ccc(-c4cccc(-c5cccc(-c6cccc(-c7ccccn7)n6)c5)c4)c4cccc-3c24)cc1]

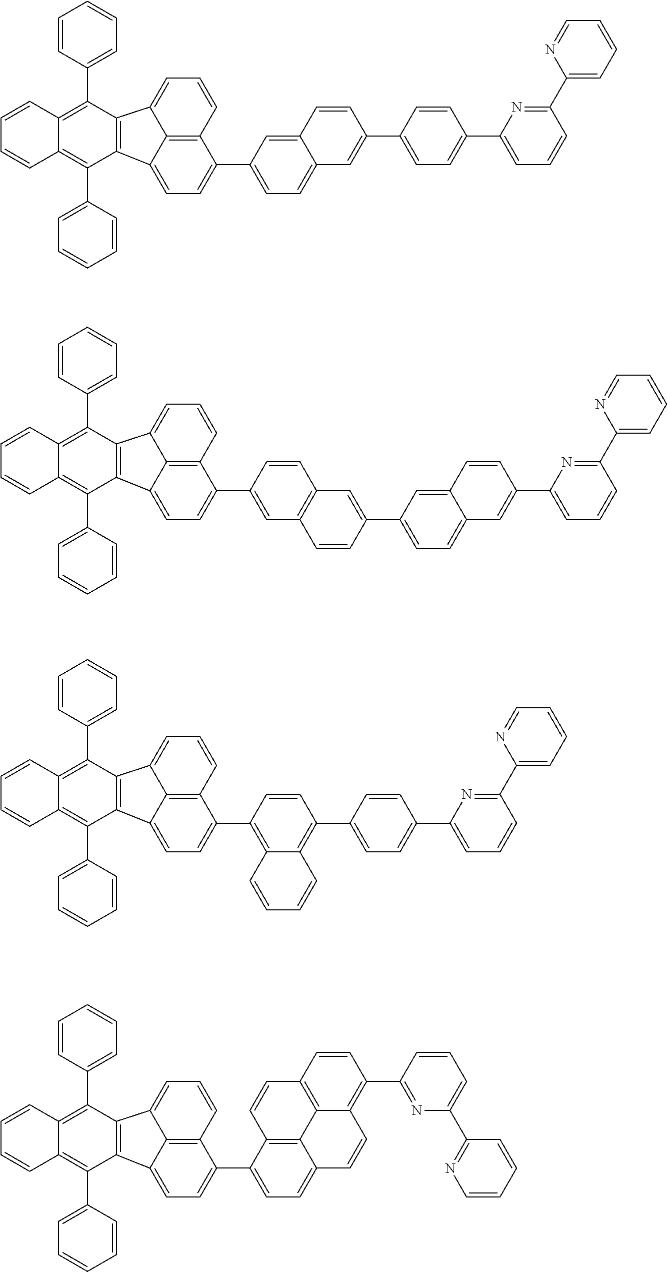 [SMILES: c1ccc(-c2c3c(c(-c4ccccc4)c4ccccc24)-c2ccc(-c4ccc5cc(-c6ccc(-c7cccc(-c8ccccn8)n7)cc6)ccc5c4)c4cccc-3c24)cc1 ; c1ccc(-c2c3c(c(-c4ccccc4)c4ccccc24)-c2ccc(-c4ccc5cc(-c6ccc7cc(-c8cccc(-c9ccccn9)n8)ccc7c6)ccc5c4)c4cccc-3c24)cc1 ; c1ccc(-c2c3c(c(-c4ccccc4)c4ccccc24)-c2ccc(-c4ccc(-c5ccc(-c6cccc(-c7ccccn7)n6)cc5)c5ccccc45)c4cccc-3c24)cc1 ; c1ccc(-c2c3c(c(-c4ccccc4)c4ccccc24)-c2ccc(-c4ccc5ccc6c(-c7cccc(-c8ccccn8)n7)ccc7ccc4c5c76)c4cccc-3c24)cc1]

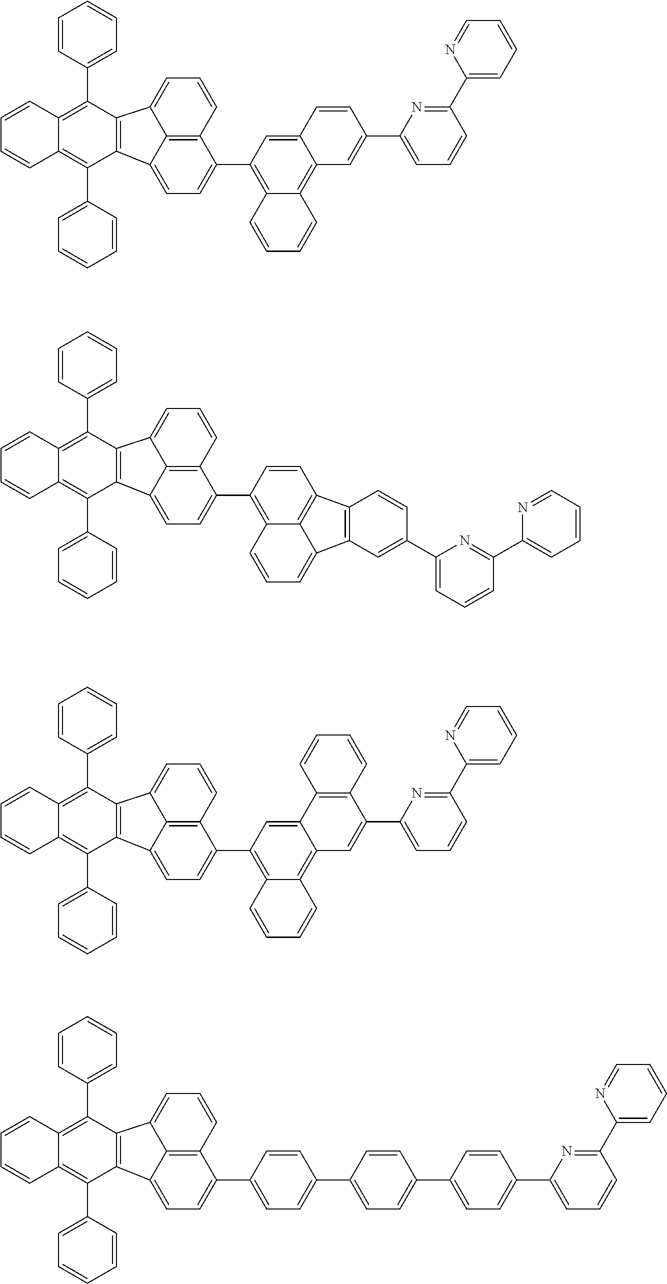 [SMILES: c1ccc(-c2c3c(c(-c4ccccc4)c4ccccc24)-c2ccc(-c4cc5ccc(-c6cccc(-c7ccccn7)n6)cc5c5ccccc45)c4cccc-3c24)cc1 ; c1ccc(-c2c3c(c(-c4ccccc4)c4ccccc24)-c2ccc(-c4ccc5c6c(cccc46)-c4cc(-c6cccc(-c7ccccn7)n6)ccc4-5)c4cccc-3c24)cc1 ; c1ccc(-c2c3c(c(-c4ccccc4)c4ccccc24)-c2ccc(-c4cc5c6ccccc6c(-c6cccc(-c7ccccn7)n6)cc5c5ccccc45)c4cccc-3c24)cc1 ; c1ccc(-c2c3c(c(-c4ccccc4)c4ccccc24)-c2ccc(-c4ccc(-c5ccc(-c6ccc(-c7cccc(-c8ccccn8)n7)cc6)cc5)cc4)c4cccc-3c24)cc1]

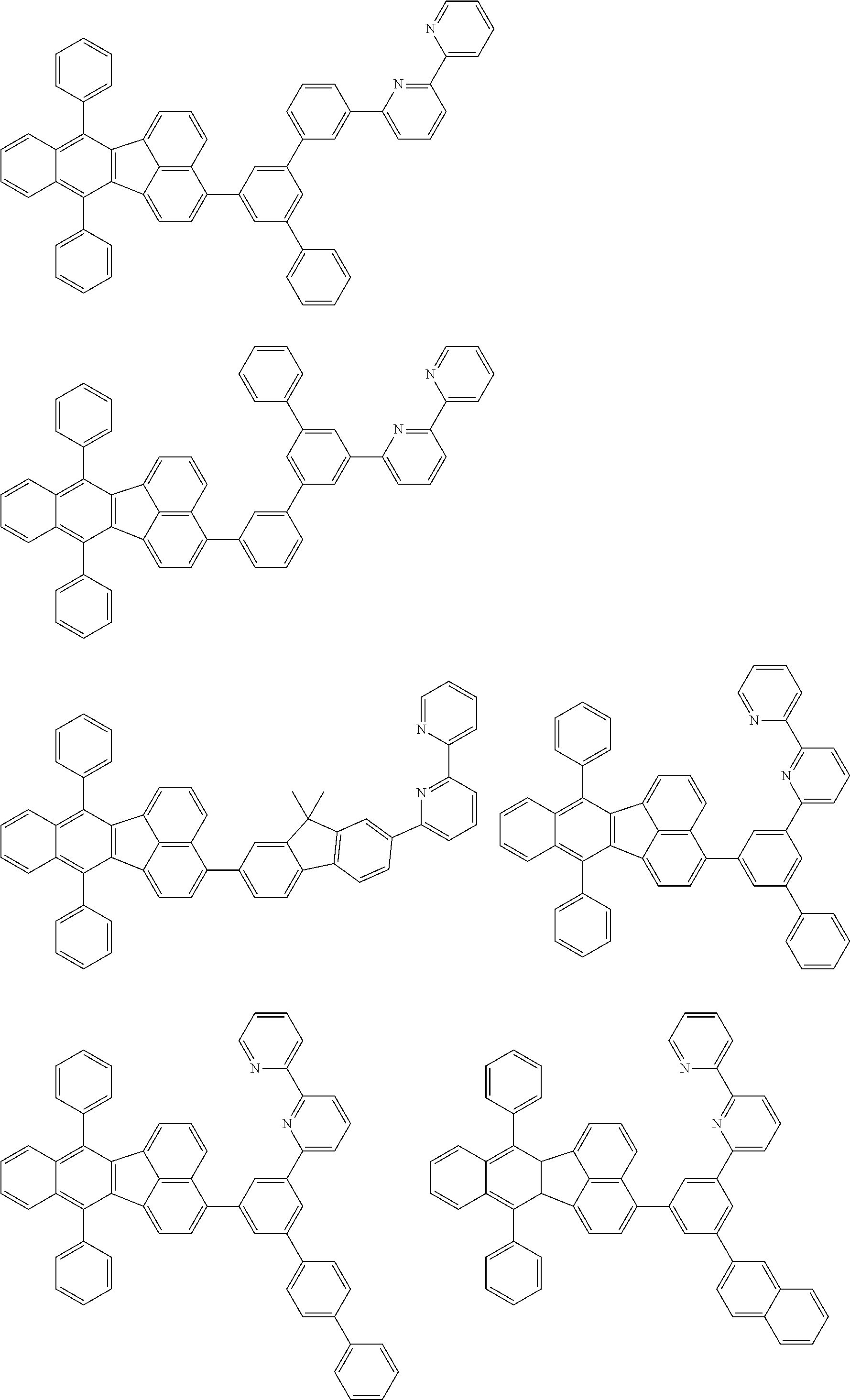

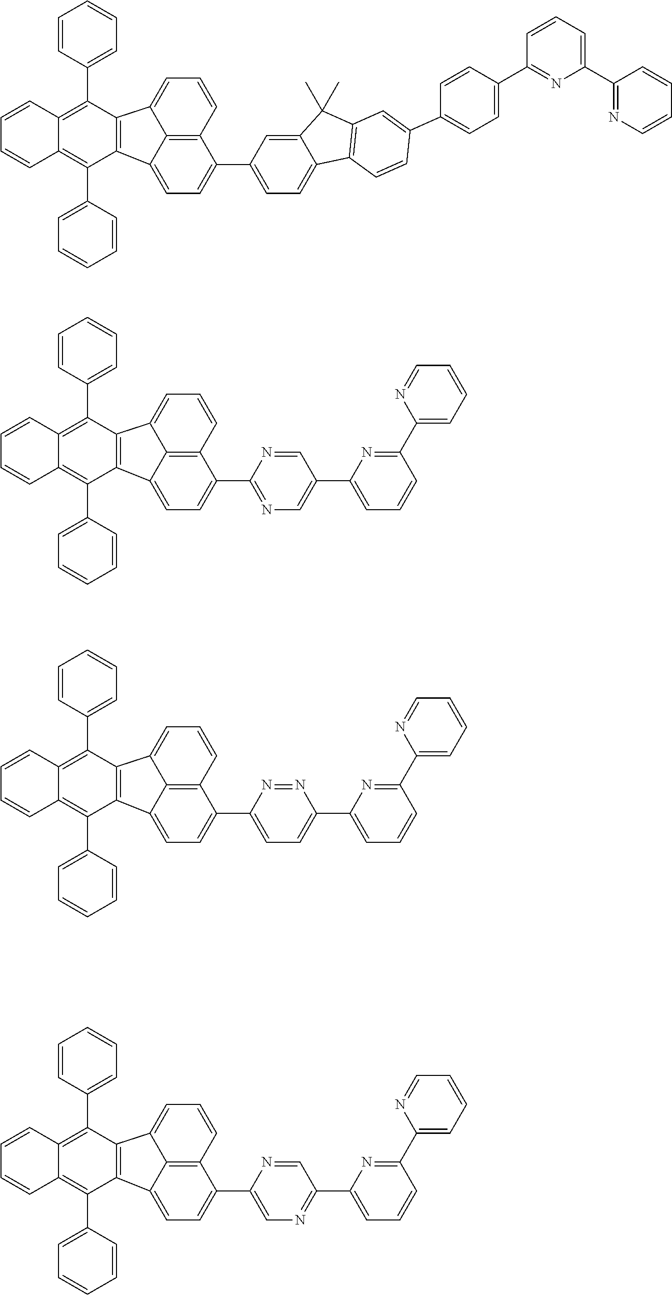 [SMILES: CC1(C)c2cc(-c3ccc(-c4cccc(-c5ccccn5)n4)cc3)ccc2-c2ccc(-c3ccc4c5c(cccc35)-c3c-4c(-c4ccccc4)c4ccccc4c3-c3ccccc3)cc21 ; c1ccc(-c2c3c(c(-c4ccccc4)c4ccccc24)-c2ccc(-c4ncc(-c5cccc(-c6ccccn6)n5)cn4)c4cccc-3c24)cc1 ; c1ccc(-c2c3c(c(-c4ccccc4)c4ccccc24)-c2ccc(-c4ccc(-c5cccc(-c6ccccn6)n5)nn4)c4cccc-3c24)cc1 ; c1ccc(-c2c3c(c(-c4ccccc4)c4ccccc24)-c2ccc(-c4cnc(-c5cccc(-c6ccccn6)n5)cn4)c4cccc-3c24)cc1]

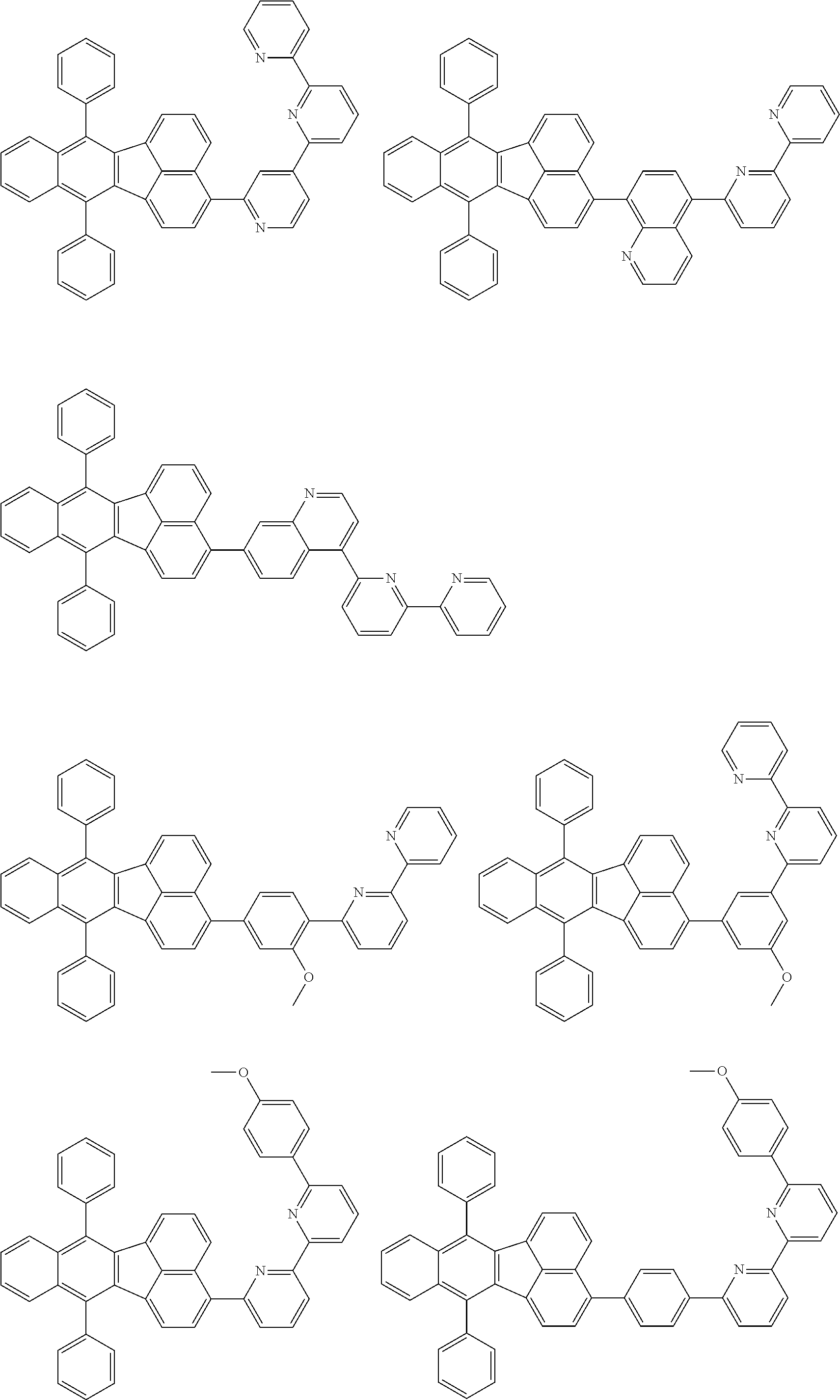

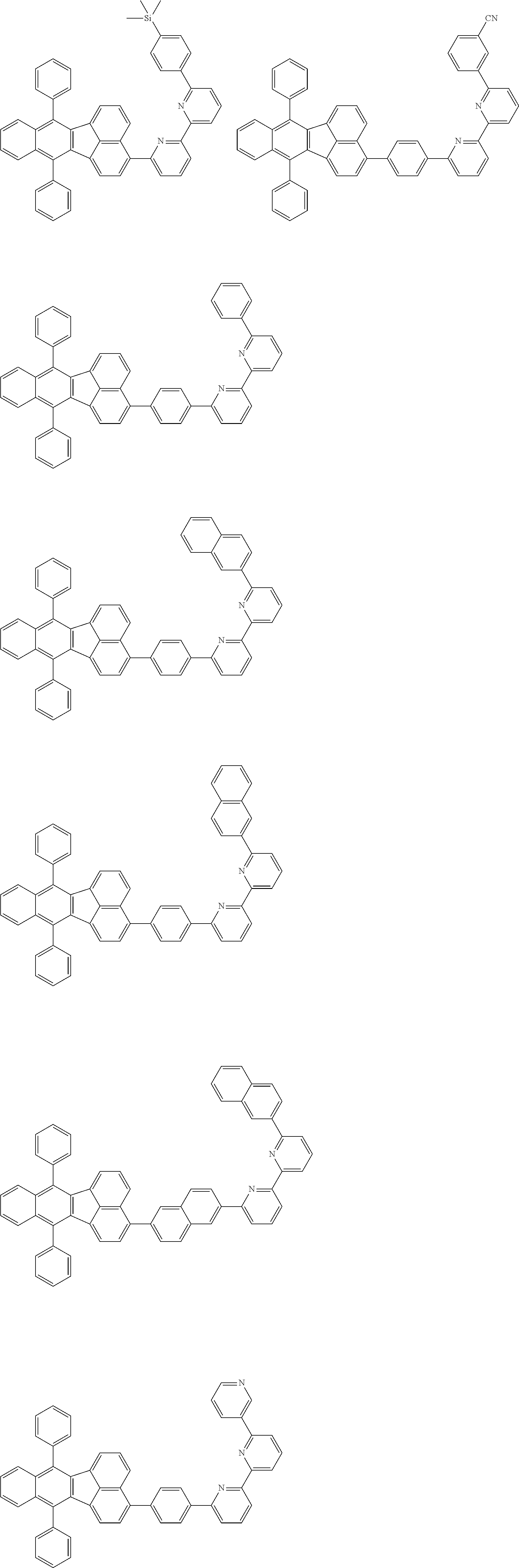

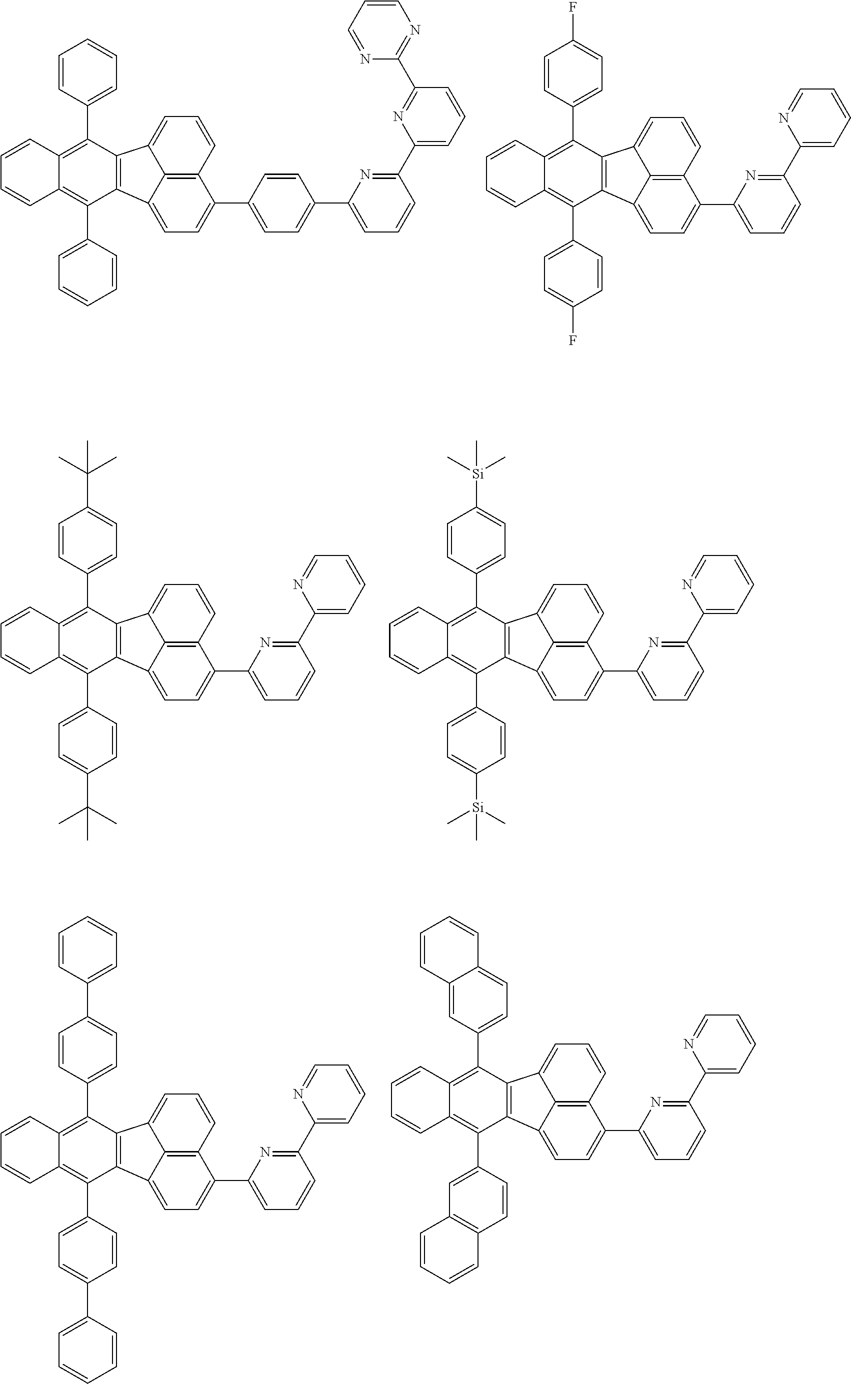

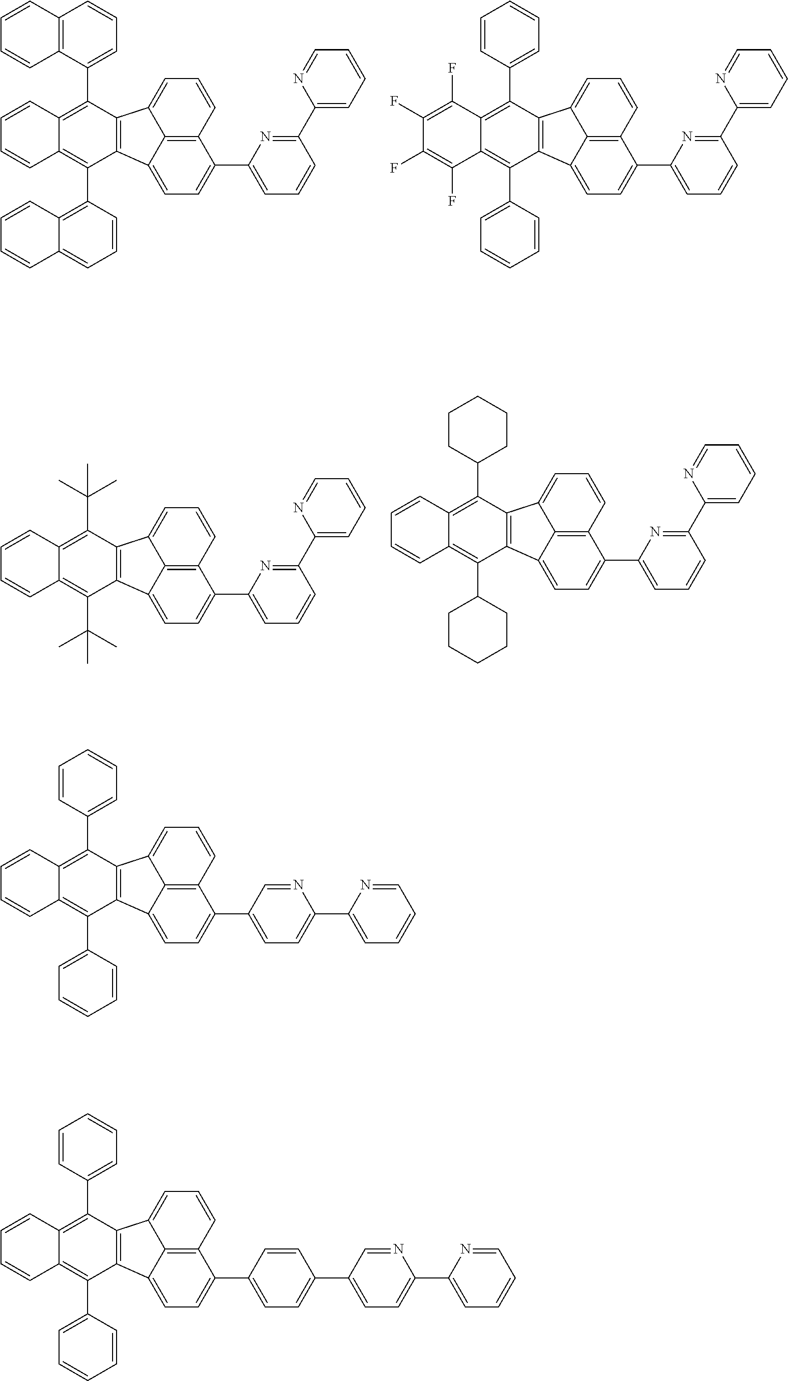

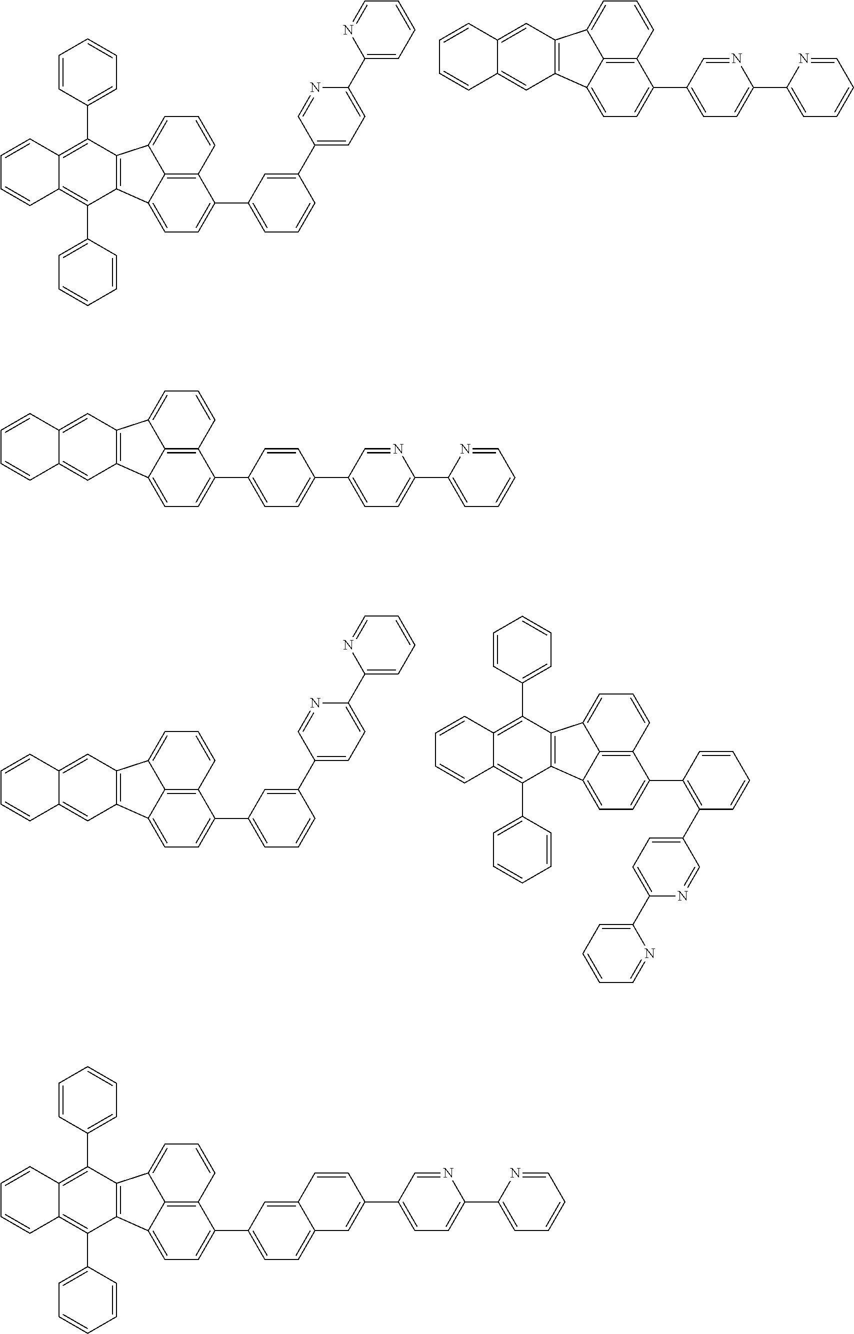

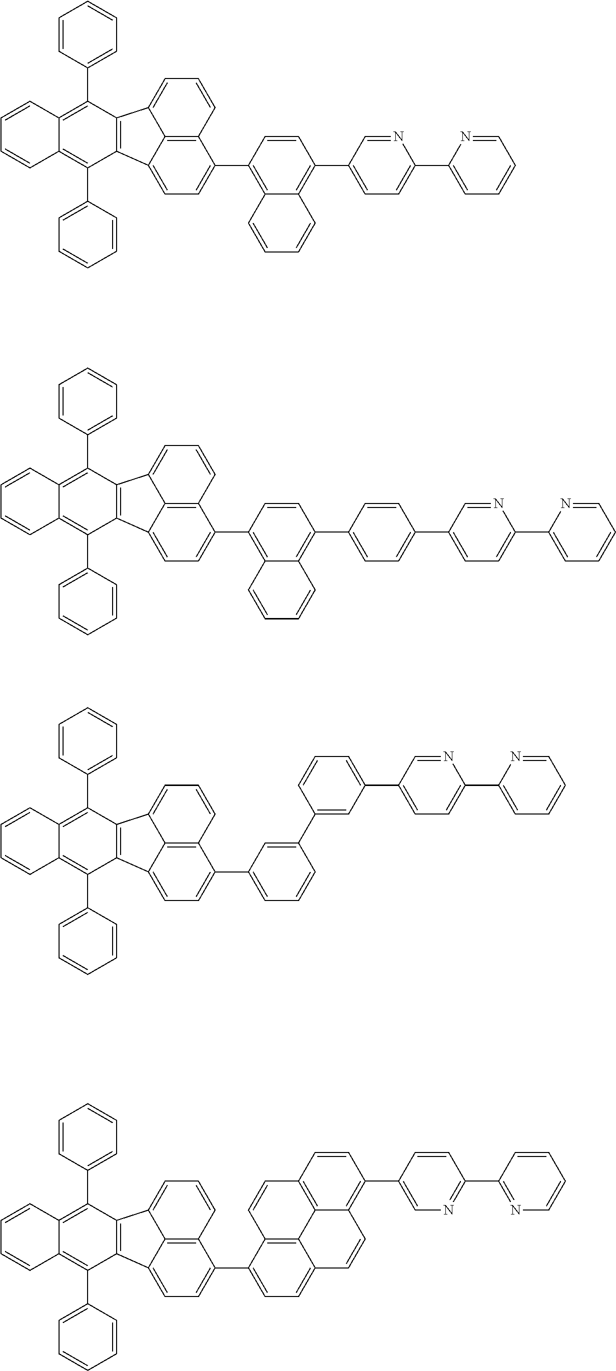 [SMILES: c1ccc(-c2c3c(c(-c4ccccc4)c4ccccc24)-c2ccc(-c4ccc(-c5ccc(-c6ccccn6)nc5)c5ccccc45)c4cccc-3c24)cc1 ; c1ccc(-c2c3c(c(-c4ccccc4)c4ccccc24)-c2ccc(-c4ccc(-c5ccc(-c6ccc(-c7ccccn7)nc6)cc5)c5ccccc45)c4cccc-3c24)cc1 ; c1ccc(-c2c3c(c(-c4ccccc4)c4ccccc24)-c2ccc(-c4cccc(-c5cccc(-c6ccc(-c7ccccn7)nc6)c5)c4)c4cccc-3c24)cc1 ; c1ccc(-c2c3c(c(-c4ccccc4)c4ccccc24)-c2ccc(-c4ccc5ccc6c(-c7ccc(-c8ccccn8)nc7)ccc7ccc4c5c76)c4cccc-3c24)cc1]

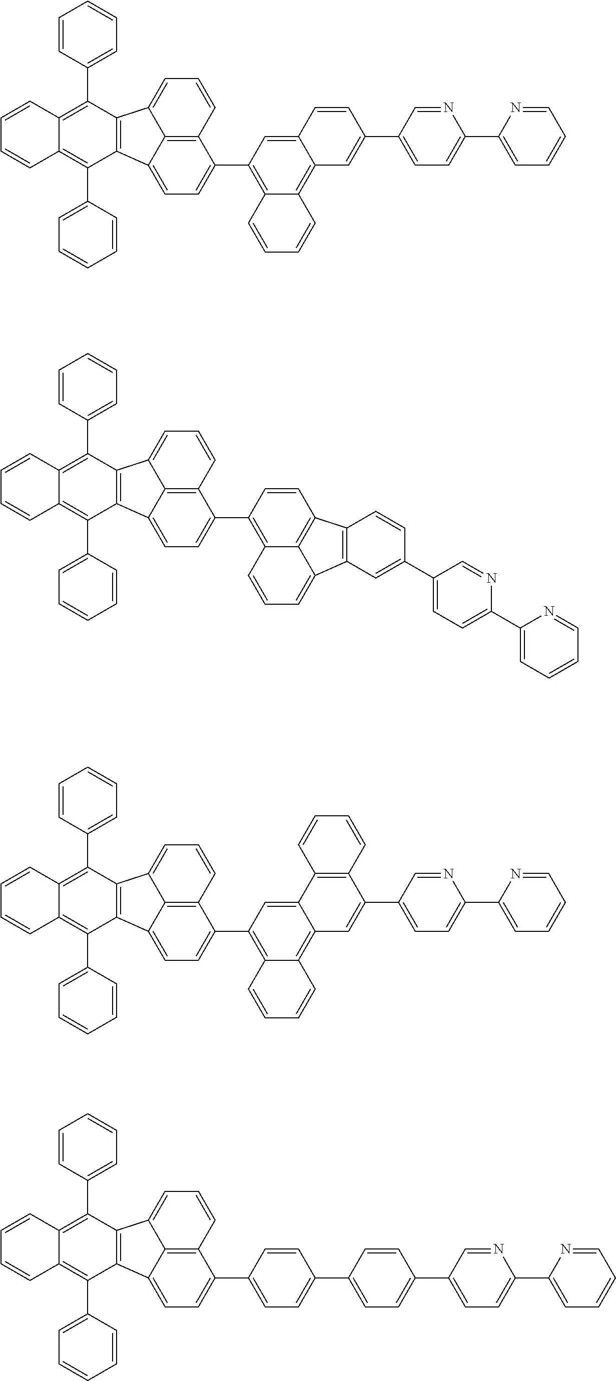 [SMILES: c1ccc(-c2c3c(c(-c4ccccc4)c4ccccc24)-c2ccc(-c4cc5ccc(-c6ccc(-c7ccccn7)nc6)cc5c5ccccc45)c4cccc-3c24)cc1 ; c1ccc(-c2c3c(c(-c4ccccc4)c4ccccc24)-c2ccc(-c4ccc5c6c(cccc46)-c4cc(-c6ccc(-c7ccccn7)nc6)ccc4-5)c4cccc-3c24)cc1 ; c1ccc(-c2c3c(c(-c4ccccc4)c4ccccc24)-c2ccc(-c4cc5c6ccccc6c(-c6ccc(-c7ccccn7)nc6)cc5c5ccccc45)c4cccc-3c24)cc1 ; c1ccc(-c2c3c(c(-c4ccccc4)c4ccccc24)-c2ccc(-c4ccc(-c5ccc(-c6ccc(-c7ccccn7)nc6)cc5)cc4)c4cccc-3c24)cc1]

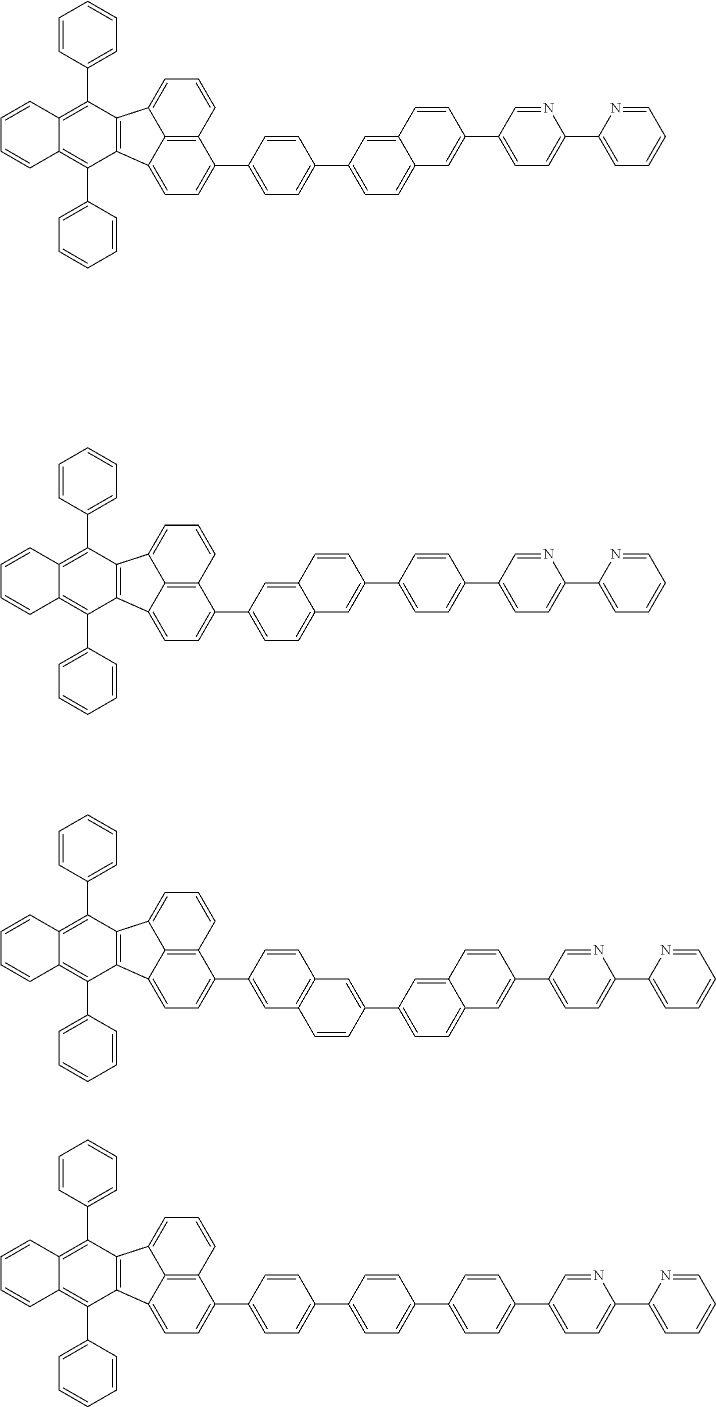 [SMILES: c1ccc(-c2c3c(c(-c4ccccc4)c4ccccc24)-c2ccc(-c4ccc(-c5ccc6cc(-c7ccc(-c8ccccn8)nc7)ccc6c5)cc4)c4cccc-3c24)cc1 ; c1ccc(-c2c3c(c(-c4ccccc4)c4ccccc24)-c2ccc(-c4ccc5cc(-c6ccc(-c7ccc(-c8ccccn8)nc7)cc6)ccc5c4)c4cccc-3c24)cc1 ; c1ccc(-c2c3c(c(-c4ccccc4)c4ccccc24)-c2ccc(-c4ccc5cc(-c6ccc7cc(-c8ccc(-c9ccccn9)nc8)ccc7c6)ccc5c4)c4cccc-3c24)cc1 ; c1ccc(-c2c3c(c(-c4ccccc4)c4ccccc24)-c2ccc(-c4ccc(-c5ccc(-c6ccc(-c7ccc(-c8ccccn8)nc7)cc6)cc5)cc4)c4cccc-3c24)cc1]

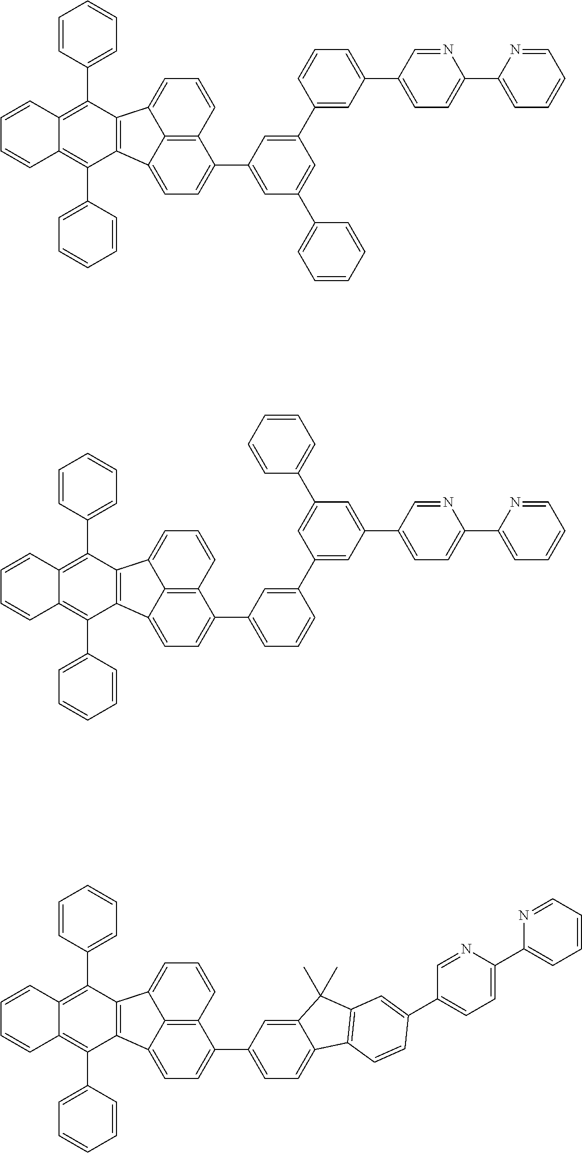 [SMILES: c1ccc(-c2cc(-c3cccc(-c4ccc(-c5ccccn5)nc4)c3)cc(-c3ccc4c5c(cccc35)-c3c-4c(-c4ccccc4)c4ccccc4c3-c3ccccc3)c2)cc1 ; c1ccc(-c2cc(-c3ccc(-c4ccccn4)nc3)cc(-c3cccc(-c4ccc5c6c(cccc46)-c4c-5c(-c5ccccc5)c5ccccc5c4-c4ccccc4)c3)c2)cc1 ; CC1(C)c2cc(-c3ccc(-c4ccccn4)nc3)ccc2-c2ccc(-c3ccc4c5c(cccc35)-c3c-4c(-c4ccccc4)c4ccccc4c3-c3ccccc3)cc21]

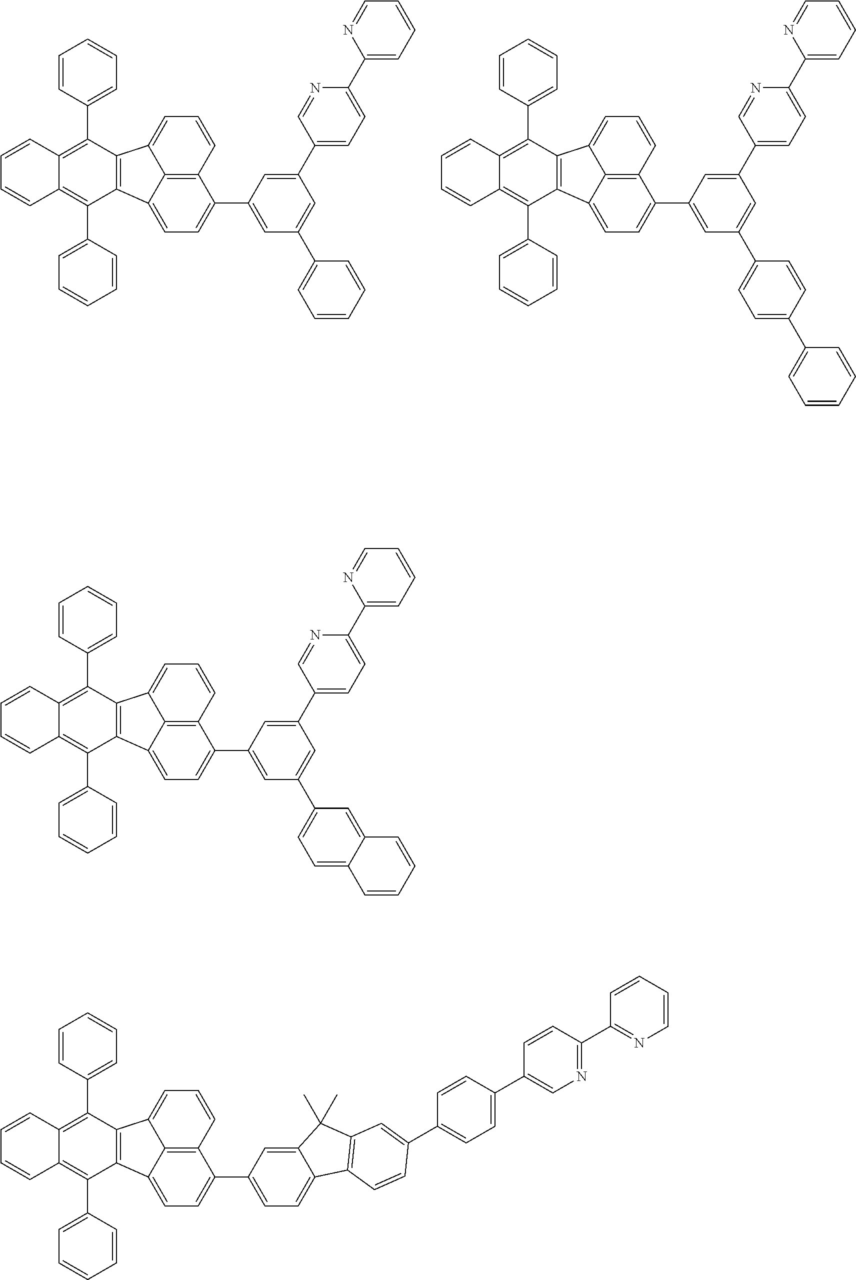 [SMILES: c1ccc(-c2cc(-c3ccc(-c4ccccn4)nc3)cc(-c3ccc4c5c(cccc35)-c3c-4c(-c4ccccc4)c4ccccc4c3-c3ccccc3)c2)cc1 ; c1ccc(-c2ccc(-c3cc(-c4ccc(-c5ccccn5)nc4)cc(-c4ccc5c6c(cccc46)-c4c-5c(-c5ccccc5)c5ccccc5c4-c4ccccc4)c3)cc2)cc1 ; c1ccc(-c2c3c(c(-c4ccccc4)c4ccccc24)-c2ccc(-c4cc(-c5ccc(-c6ccccn6)nc5)cc(-c5ccc6ccccc6c5)c4)c4cccc-3c24)cc1 ; CC1(C)c2cc(-c3ccc(-c4ccc(-c5ccccn5)nc4)cc3)ccc2-c2ccc(-c3ccc4c5c(cccc35)-c3c-4c(-c4ccccc4)c4ccccc4c3-c3ccccc3)cc21]

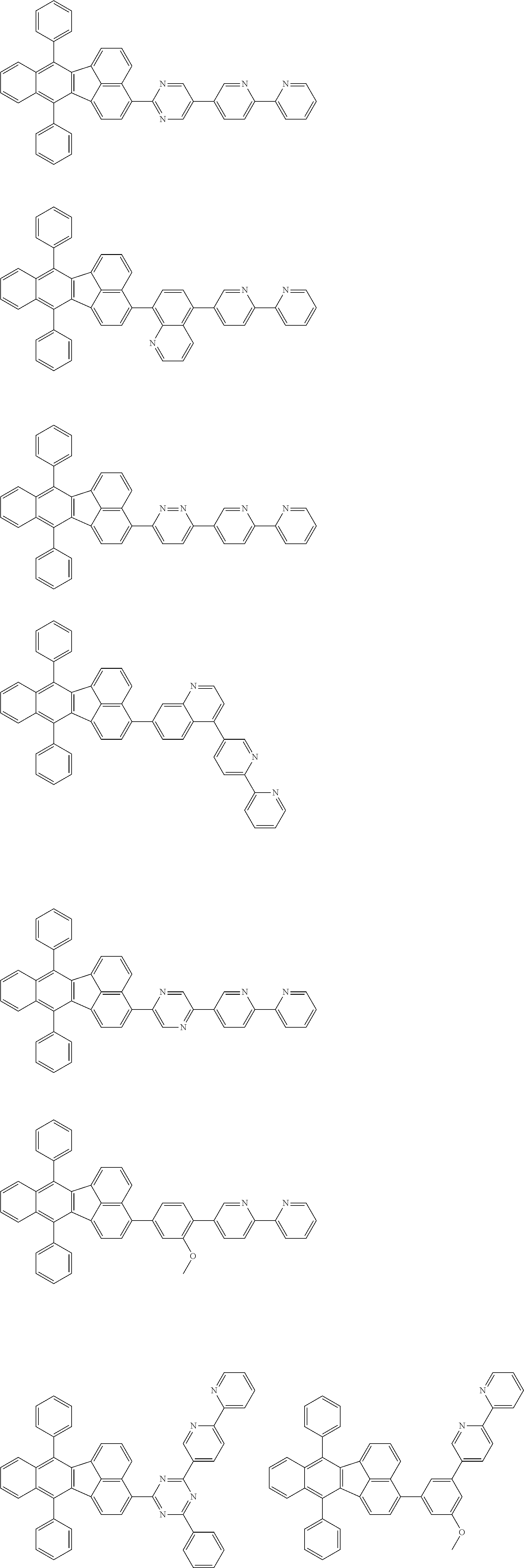

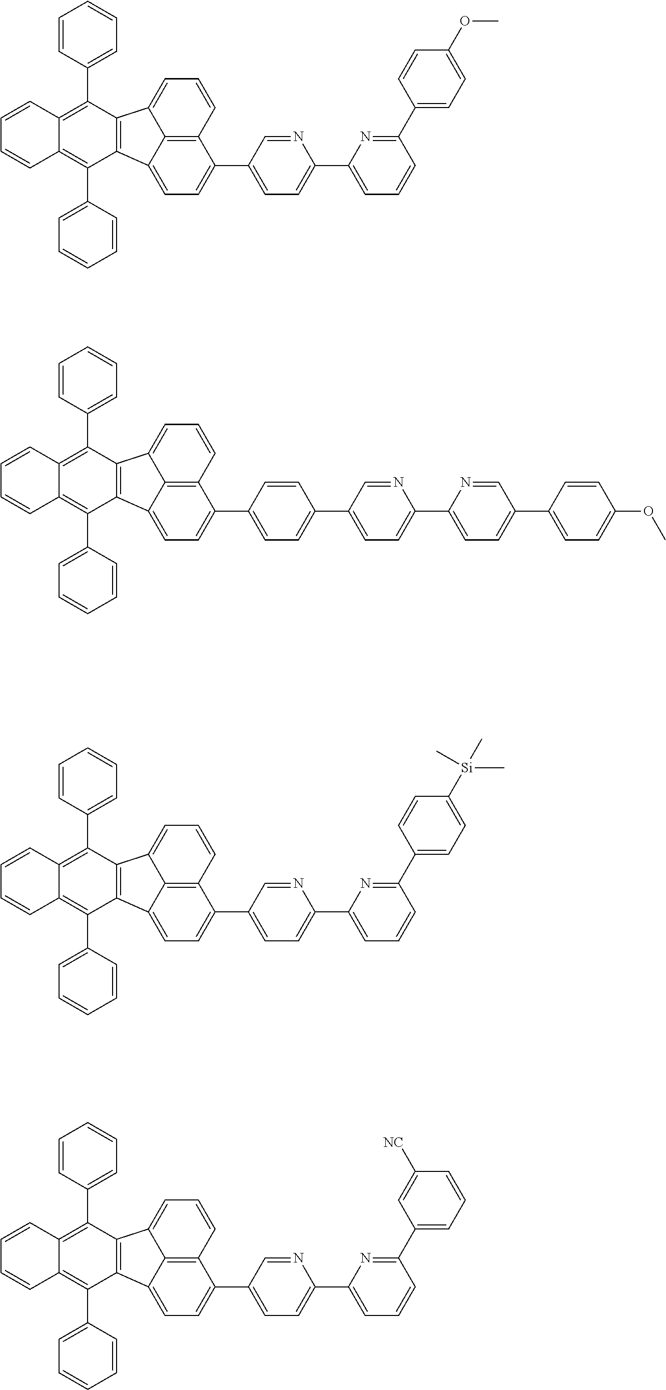 [SMILES: COc1ccc(-c2cccc(-c3ccc(-c4ccc5c6c(cccc46)-c4c-5c(-c5ccccc5)c5ccccc5c4-c4ccccc4)cn3)n2)cc1 ; COc1ccc(-c2ccc(-c3ccc(-c4ccc(-c5ccc6c7c(cccc57)-c5c-6c(-c6ccccc6)c6ccccc6c5-c5ccccc5)cc4)cn3)nc2)cc1 ; C[Si](C)(C)c1ccc(-c2cccc(-c3ccc(-c4ccc5c6c(cccc46)-c4c-5c(-c5ccccc5)c5ccccc5c4-c4ccccc4)cn3)n2)cc1 ; N#Cc1cccc(-c2cccc(-c3ccc(-c4ccc5c6c(cccc46)-c4c-5c(-c5ccccc5)c5ccccc5c4-c4ccccc4)cn3)n2)c1]

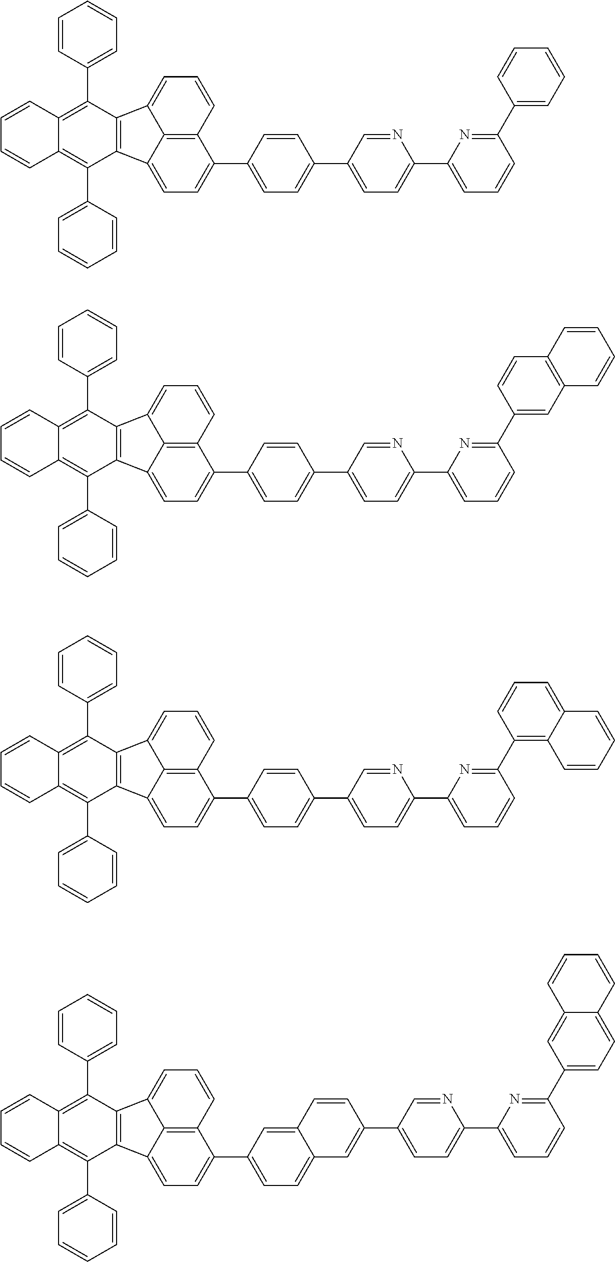

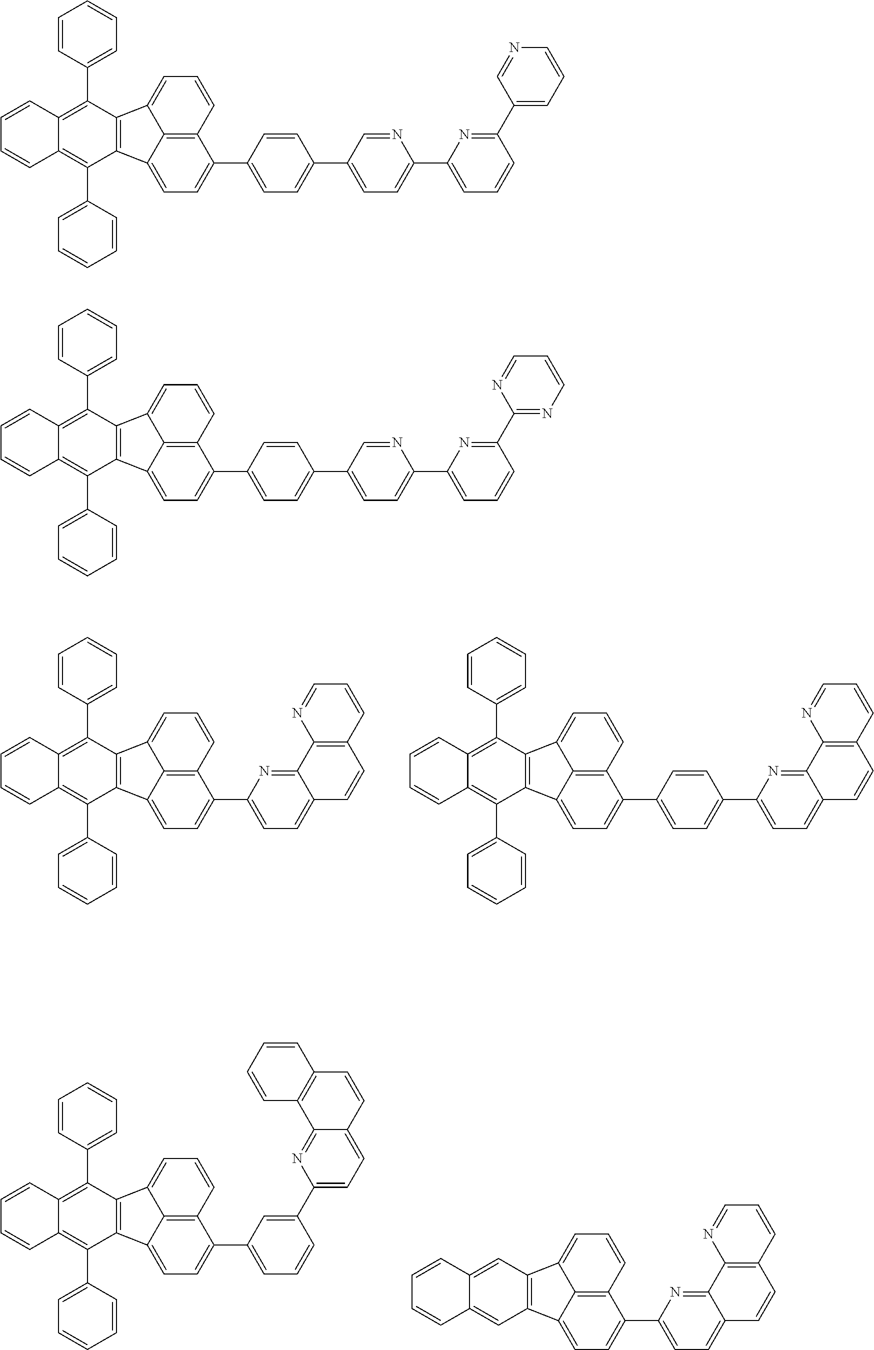

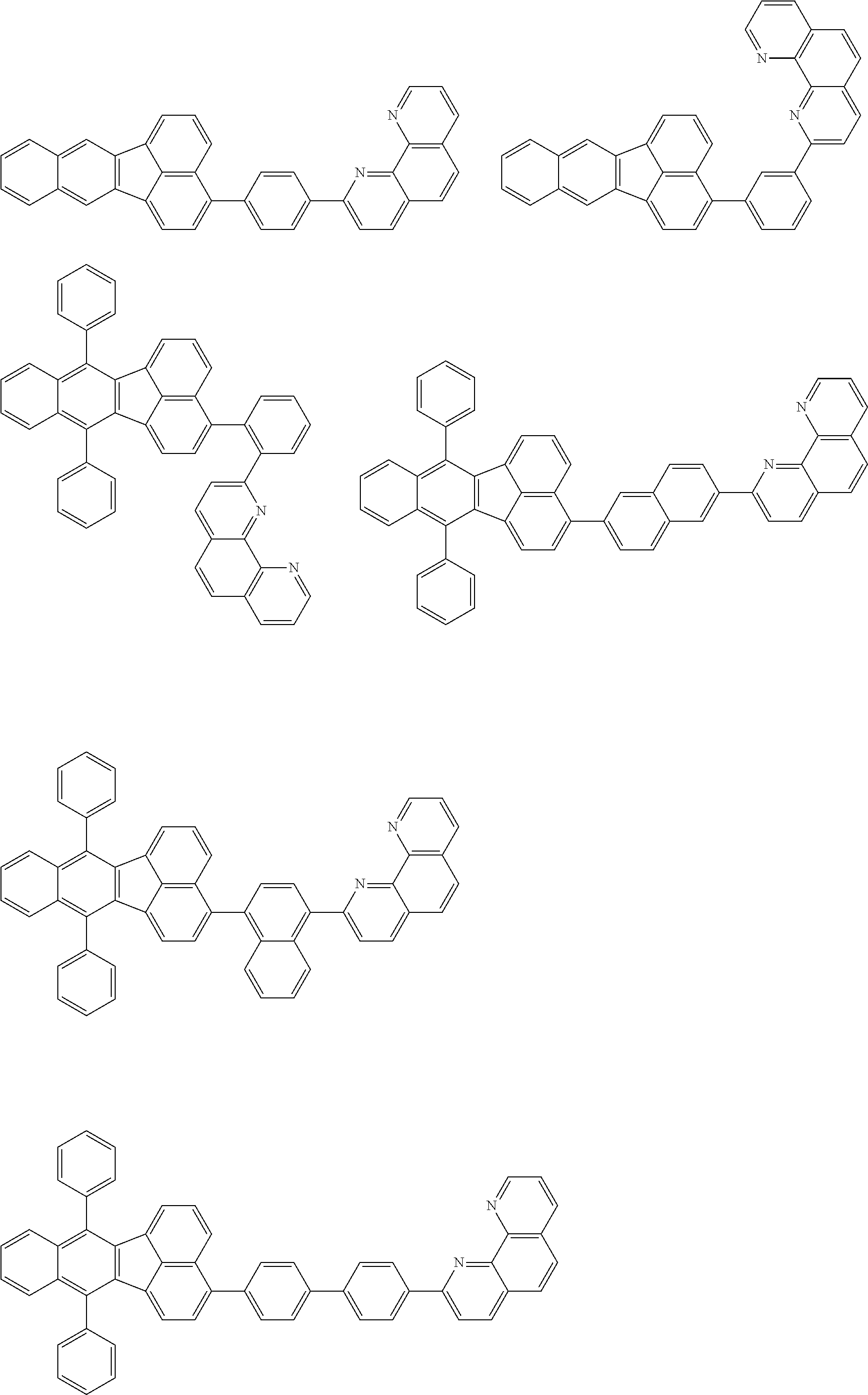

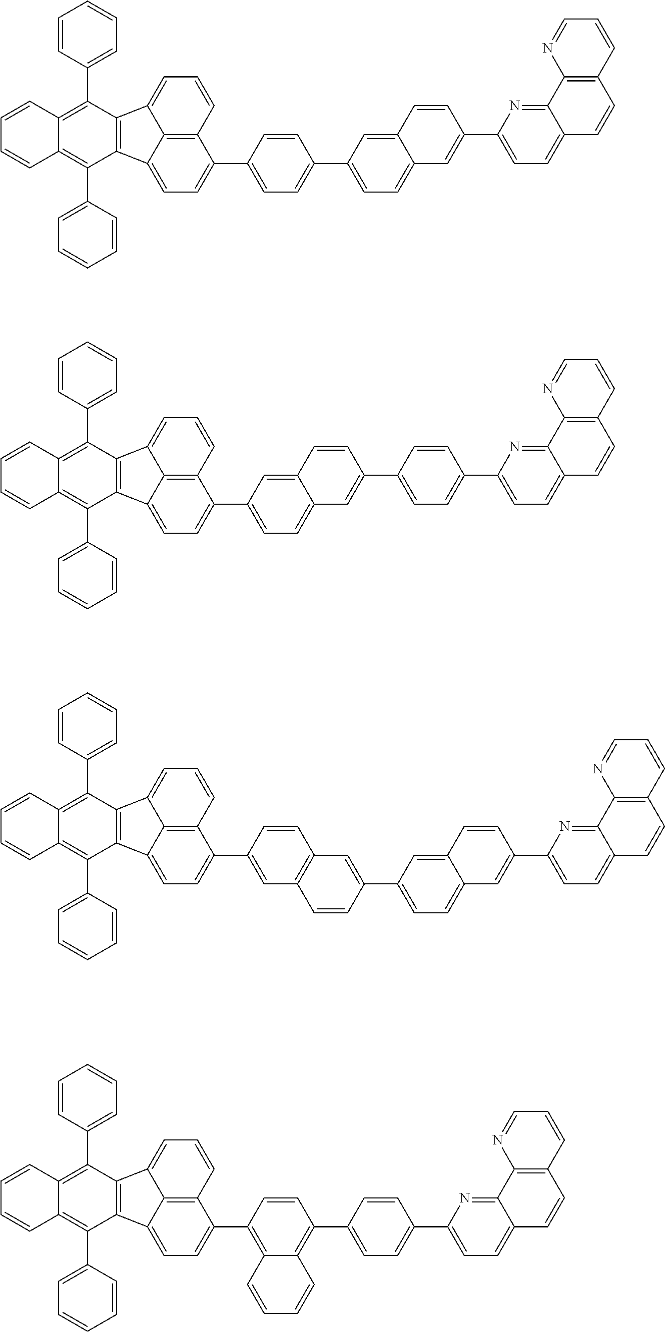 [SMILES: c1ccc(-c2c3c(c(-c4ccccc4)c4ccccc24)-c2ccc(-c4ccc(-c5ccc6cc(-c7ccc8ccc9cccnc9c8n7)ccc6c5)cc4)c4cccc-3c24)cc1 ; c1ccc(-c2c3c(c(-c4ccccc4)c4ccccc24)-c2ccc(-c4ccc5cc(-c6ccc(-c7ccc8ccc9cccnc9c8n7)cc6)ccc5c4)c4cccc-3c24)cc1 ; c1ccc(-c2c3c(c(-c4ccccc4)c4ccccc24)-c2ccc(-c4ccc5cc(-c6ccc7cc(-c8ccc9ccc%10cccnc%10c9n8)ccc7c6)ccc5c4)c4cccc-3c24)cc1 ; c1ccc(-c2c3c(c(-c4ccccc4)c4ccccc24)-c2ccc(-c4ccc(-c5ccc(-c6ccc7ccc8cccnc8c7n6)cc5)c5ccccc45)c4cccc-3c24)cc1]

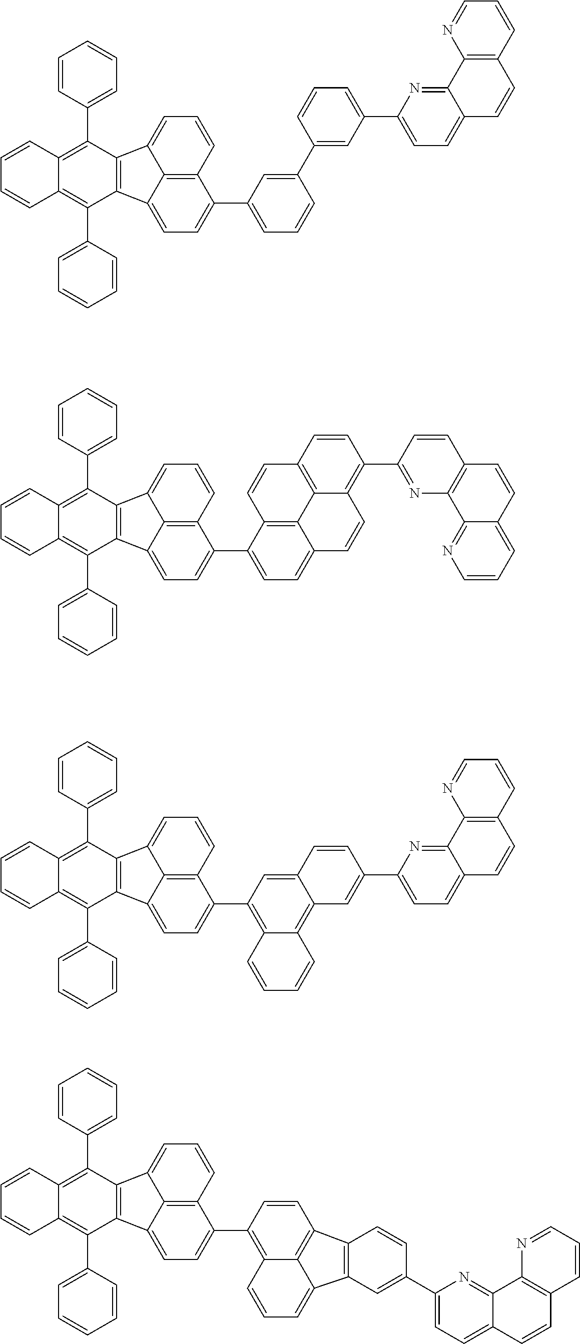 [SMILES: c1ccc(-c2c3c(c(-c4ccccc4)c4ccccc24)-c2ccc(-c4cccc(-c5cccc(-c6ccc7ccc8cccnc8c7n6)c5)c4)c4cccc-3c24)cc1 ; c1ccc(-c2c3c(c(-c4ccccc4)c4ccccc24)-c2ccc(-c4ccc5ccc6c(-c7ccc8ccc9cccnc9c8n7)ccc7ccc4c5c76)c4cccc-3c24)cc1 ; c1ccc(-c2c3c(c(-c4ccccc4)c4ccccc24)-c2ccc(-c4cc5ccc(-c6ccc7ccc8cccnc8c7n6)cc5c5ccccc45)c4cccc-3c24)cc1 ; c1ccc(-c2c3c(c(-c4ccccc4)c4ccccc24)-c2ccc(-c4ccc5c6c(cccc46)-c4cc(-c6ccc7ccc8cccnc8c7n6)ccc4-5)c4cccc-3c24)cc1]

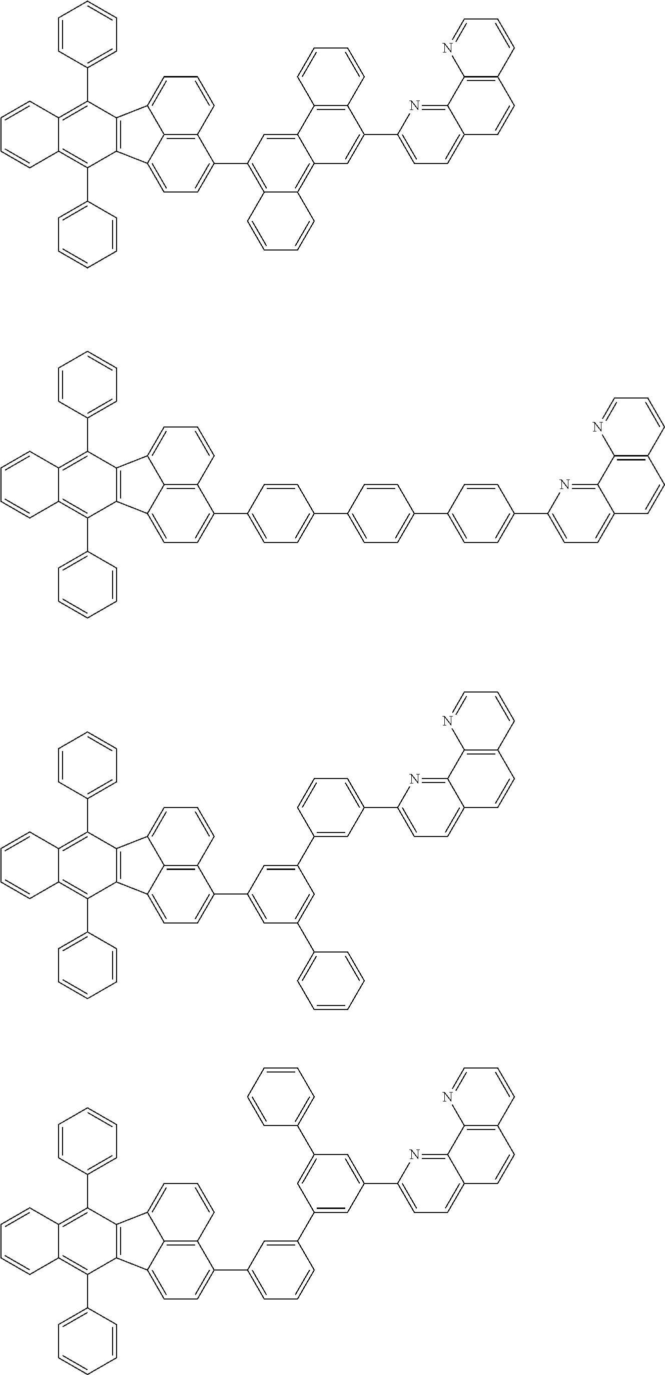 [SMILES: c1ccc(-c2c3c(c(-c4ccccc4)c4ccccc24)-c2ccc(-c4cc5c6ccccc6c(-c6ccc7ccc8cccnc8c7n6)cc5c5ccccc45)c4cccc-3c24)cc1 ; c1ccc(-c2c3c(c(-c4ccccc4)c4ccccc24)-c2ccc(-c4ccc(-c5ccc(-c6ccc(-c7ccc8ccc9cccnc9c8n7)cc6)cc5)cc4)c4cccc-3c24)cc1 ; c1ccc(-c2cc(-c3cccc(-c4ccc5ccc6cccnc6c5n4)c3)cc(-c3ccc4c5c(cccc35)-c3c-4c(-c4ccccc4)c4ccccc4c3-c3ccccc3)c2)cc1 ; c1ccc(-c2cc(-c3cccc(-c4ccc5c6c(cccc46)-c4c-5c(-c5ccccc5)c5ccccc5c4-c4ccccc4)c3)cc(-c3ccc4ccc5cccnc5c4n3)c2)cc1]

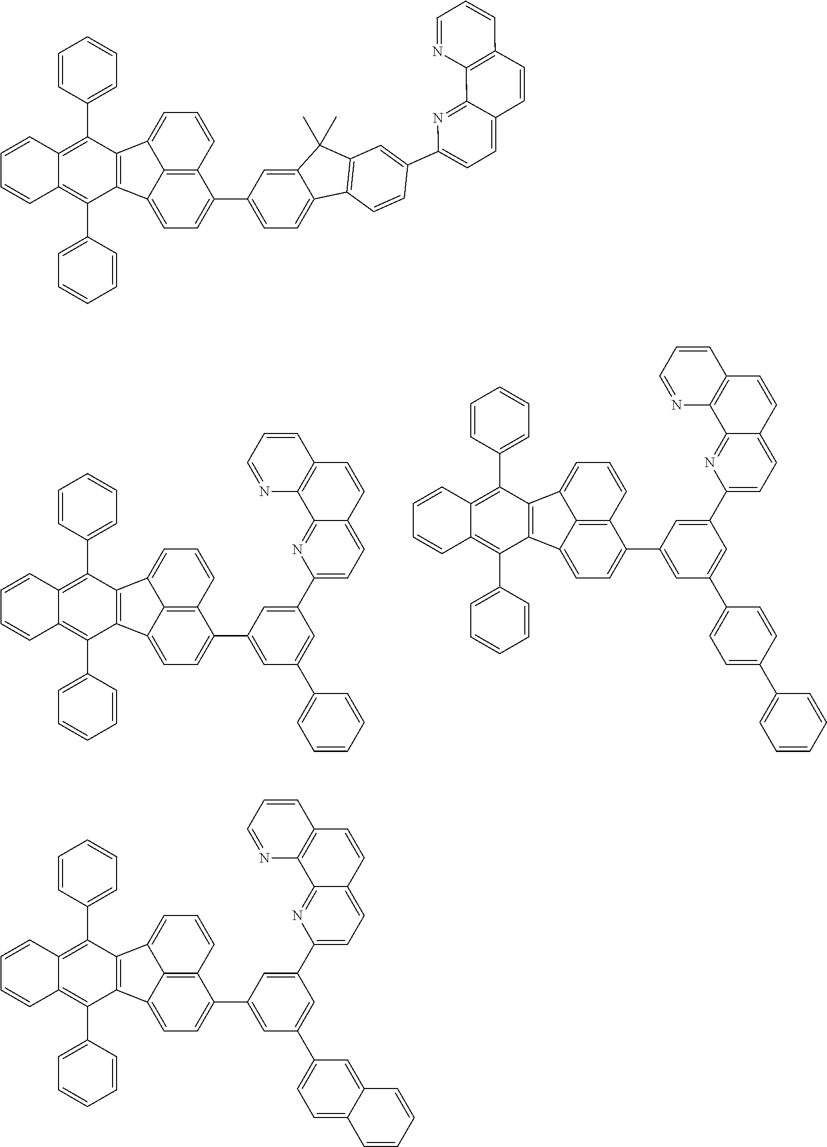 [SMILES: CC1(C)c2cc(-c3ccc4ccc5cccnc5c4n3)ccc2-c2ccc(-c3ccc4c5c(cccc35)-c3c-4c(-c4ccccc4)c4ccccc4c3-c3ccccc3)cc21 ; c1ccc(-c2cc(-c3ccc4ccc5cccnc5c4n3)cc(-c3ccc4c5c(cccc35)-c3c-4c(-c4ccccc4)c4ccccc4c3-c3ccccc3)c2)cc1 ; c1ccc(-c2ccc(-c3cc(-c4ccc5ccc6cccnc6c5n4)cc(-c4ccc5c6c(cccc46)-c4c-5c(-c5ccccc5)c5ccccc5c4-c4ccccc4)c3)cc2)cc1 ; c1ccc(-c2c3c(c(-c4ccccc4)c4ccccc24)-c2ccc(-c4cc(-c5ccc6ccccc6c5)cc(-c5ccc6ccc7cccnc7c6n5)c4)c4cccc-3c24)cc1]

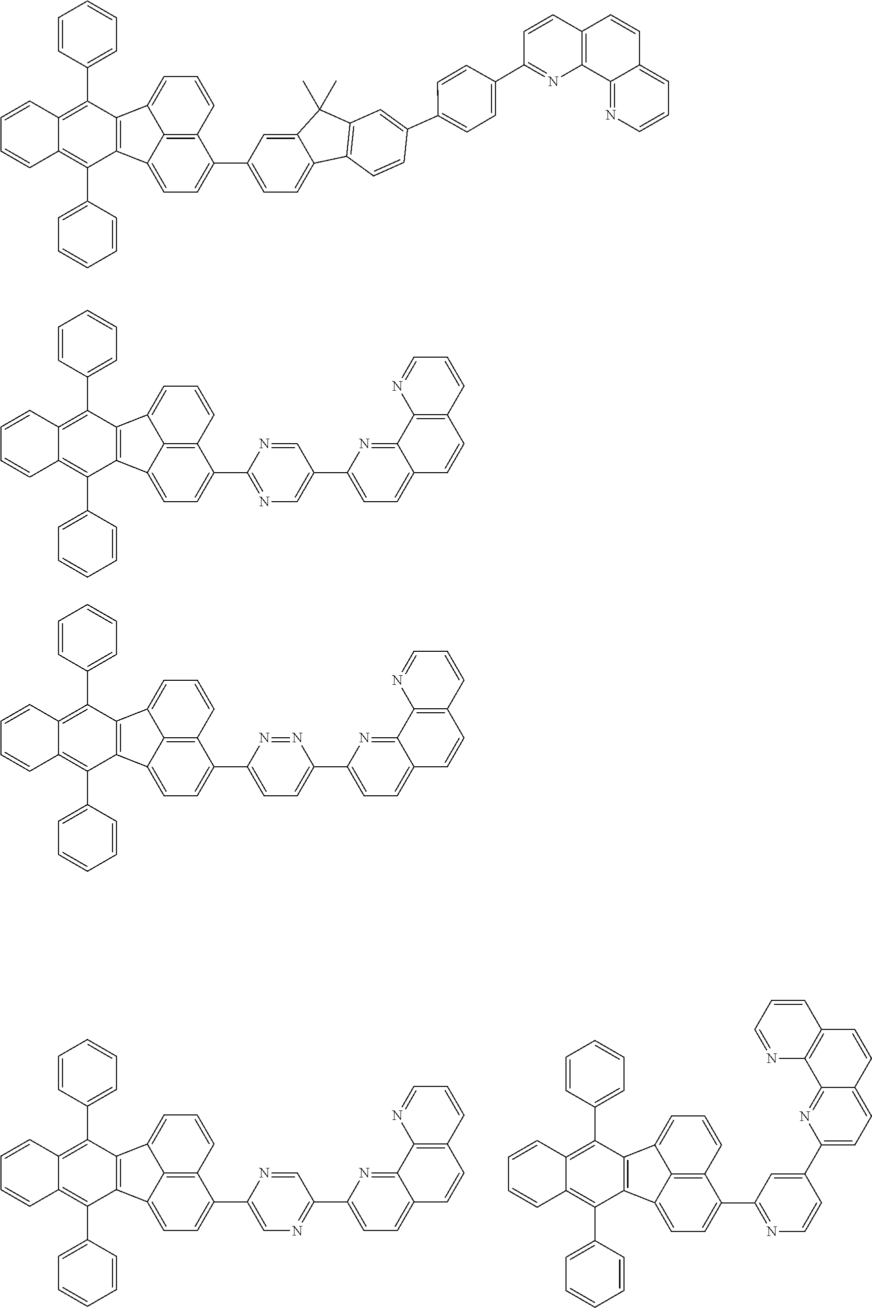

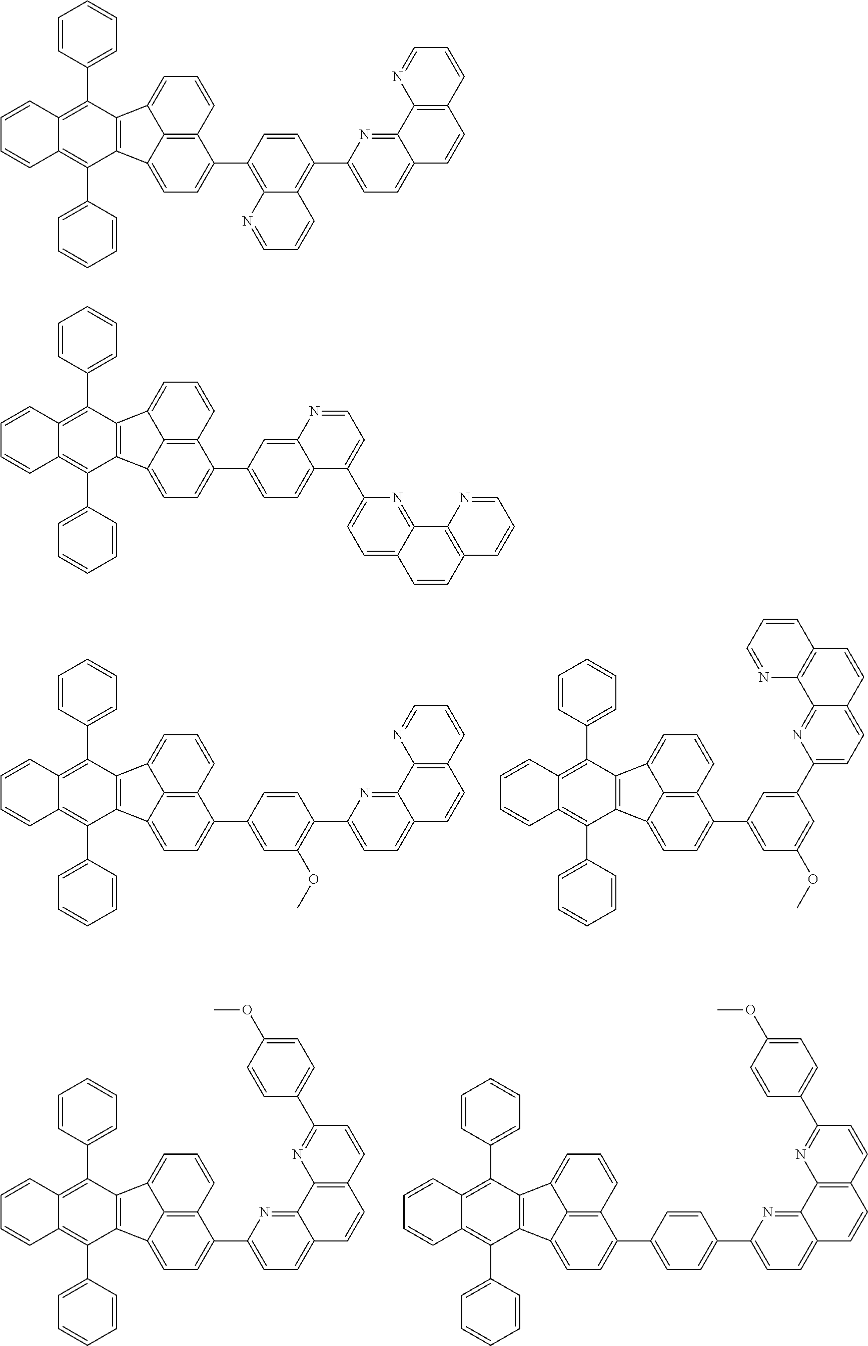

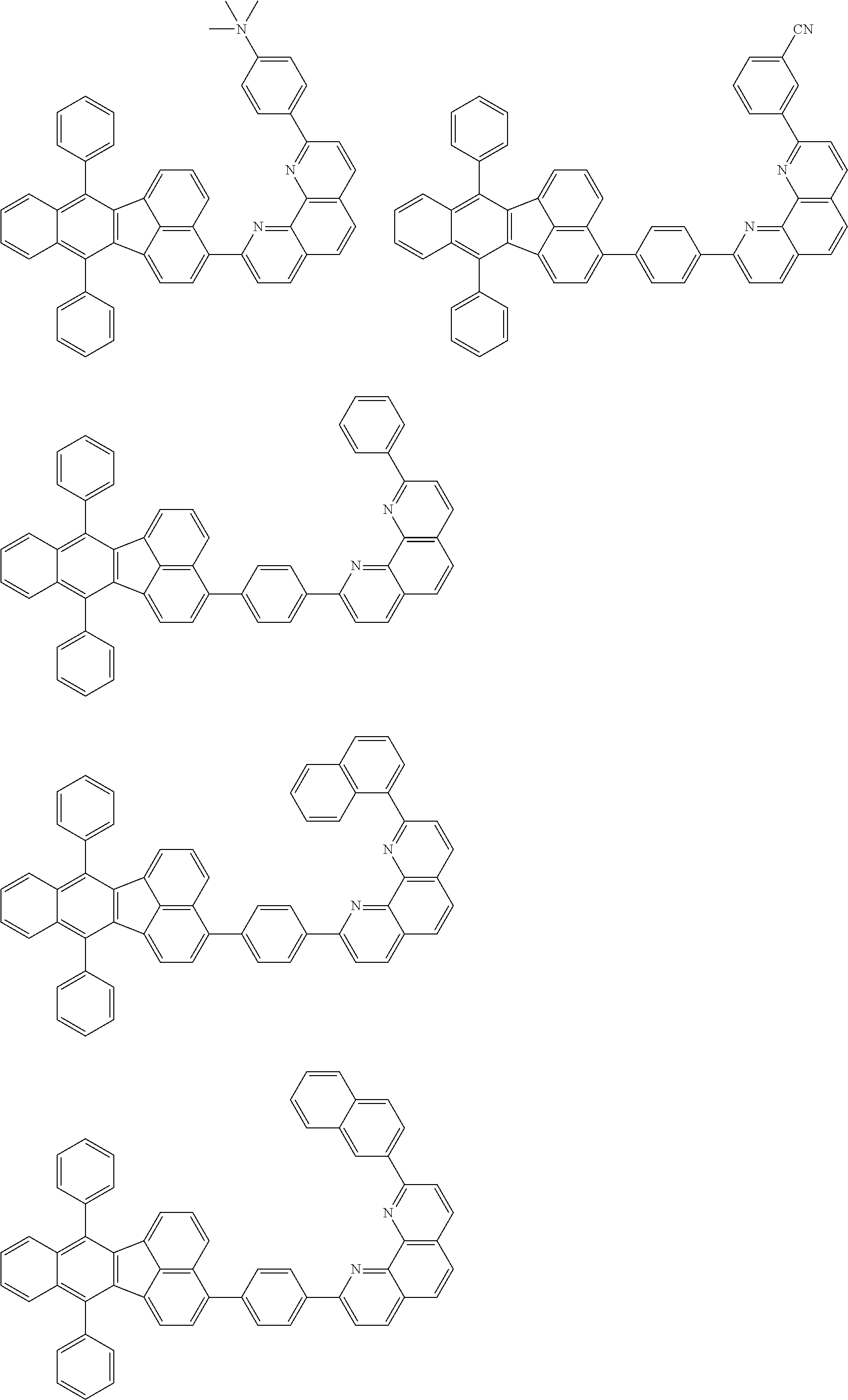

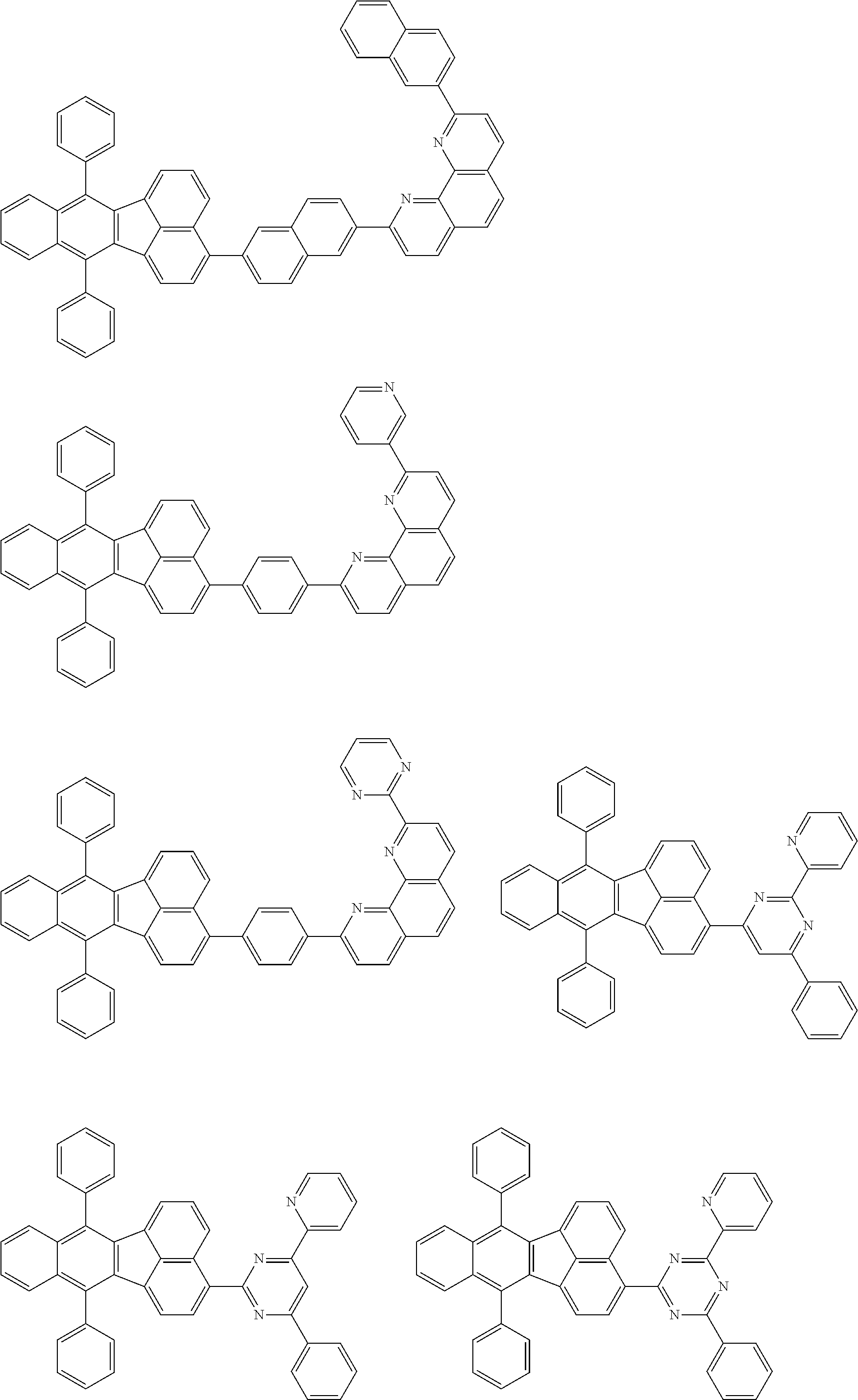

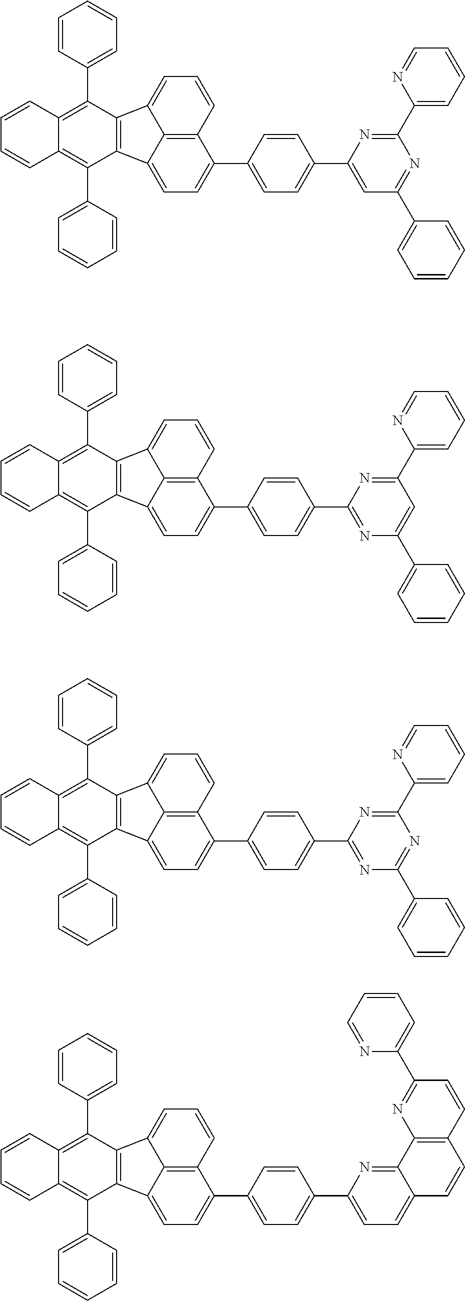

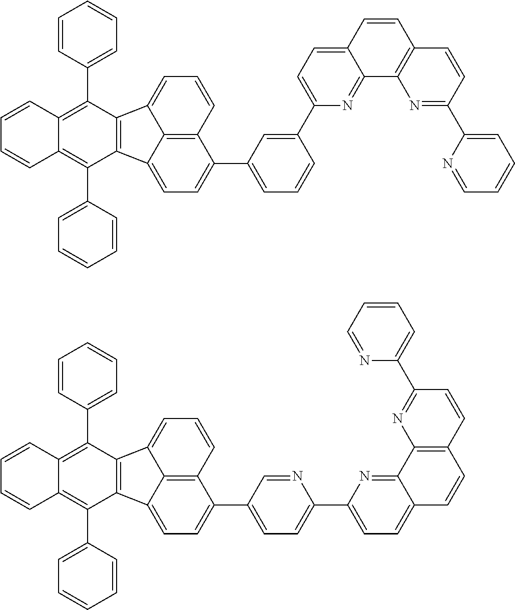 [SMILES: c1ccc(-c2c3c(c(-c4ccccc4)c4ccccc24)-c2ccc(-c4cccc(-c5ccc6ccc7ccc(-c8ccccn8)nc7c6n5)c4)c4cccc-3c24)cc1 ; c1ccc(-c2c3c(c(-c4ccccc4)c4ccccc24)-c2ccc(-c4ccc(-c5ccc6ccc7ccc(-c8ccccn8)nc7c6n5)nc4)c4cccc-3c24)cc1]

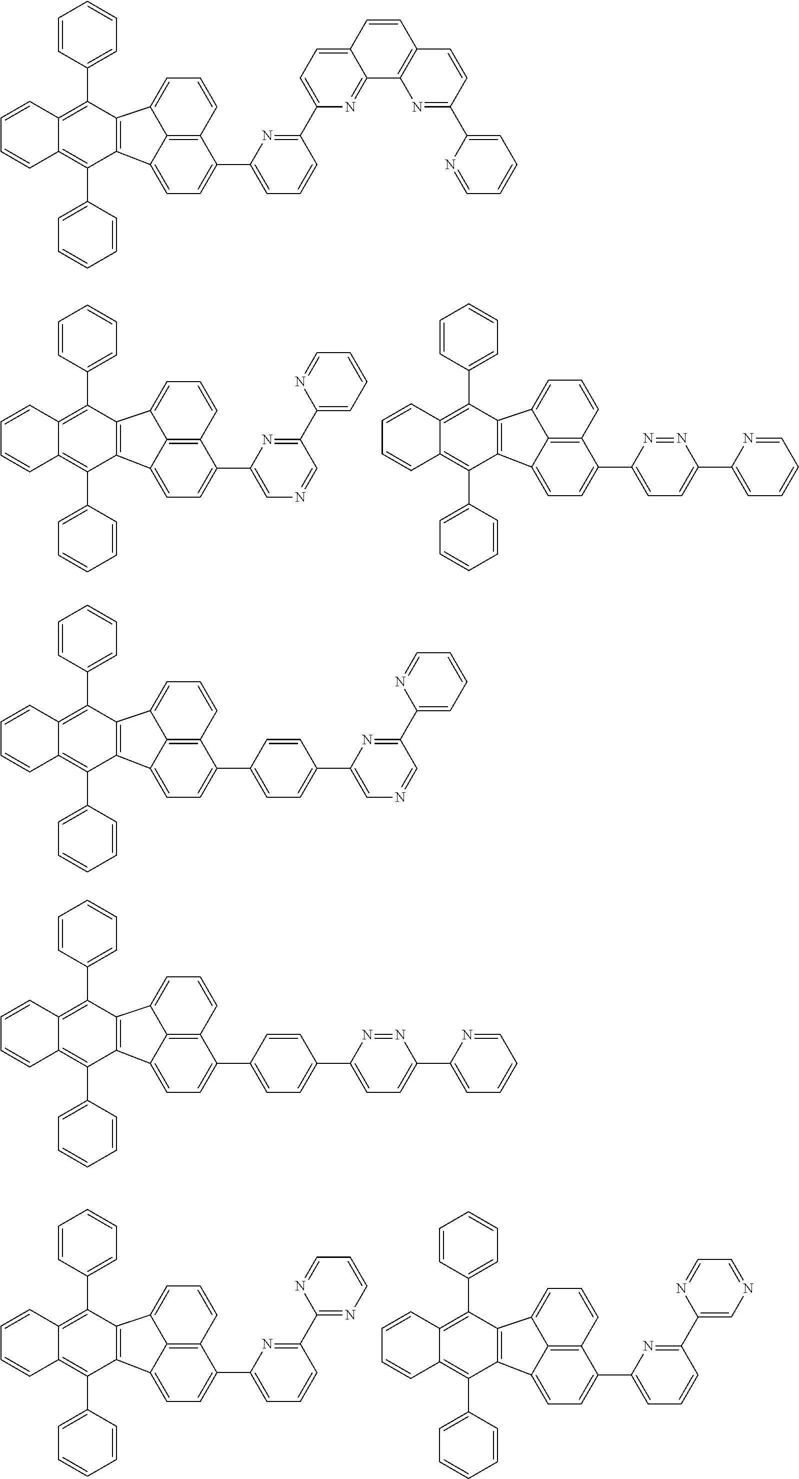

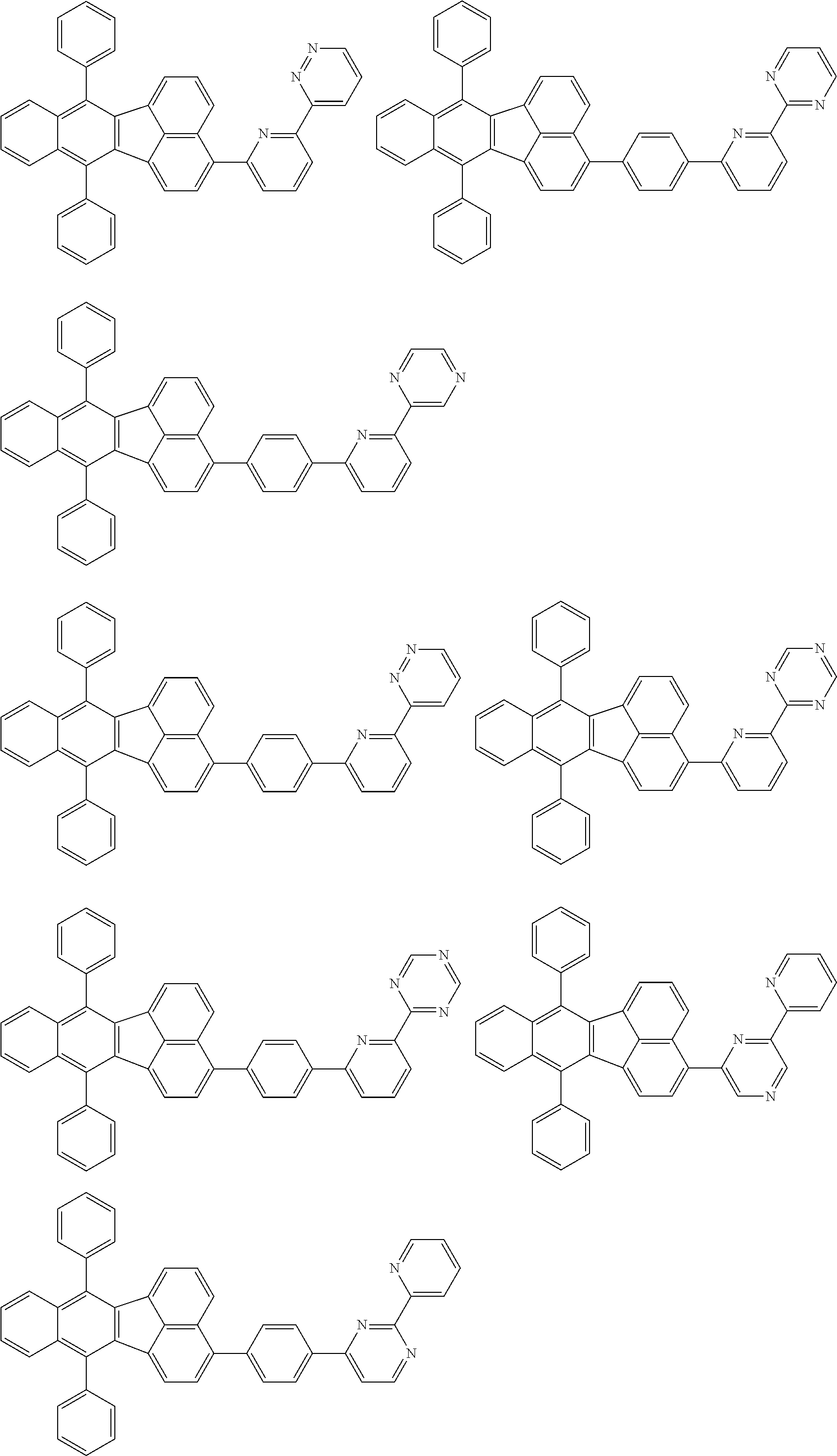

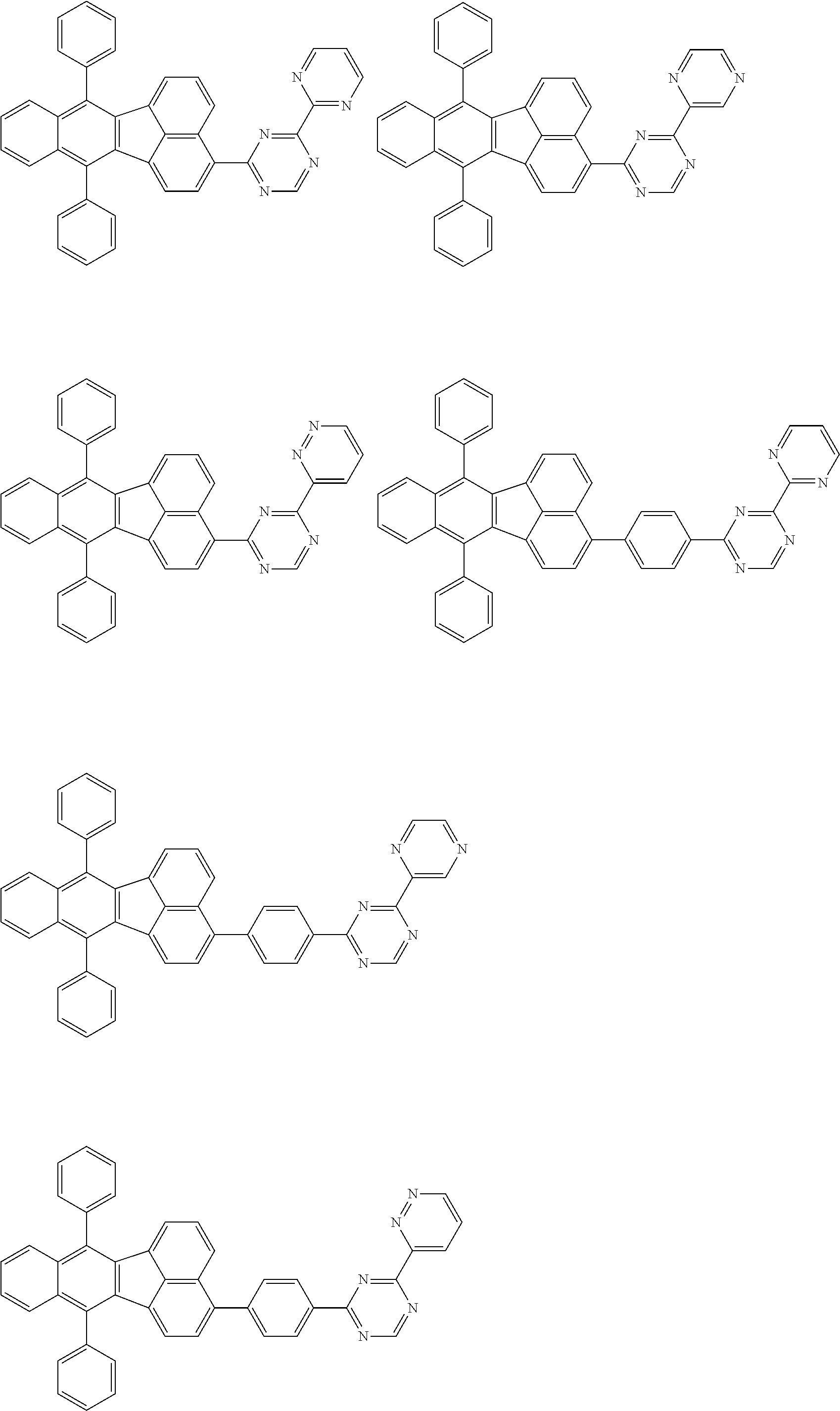

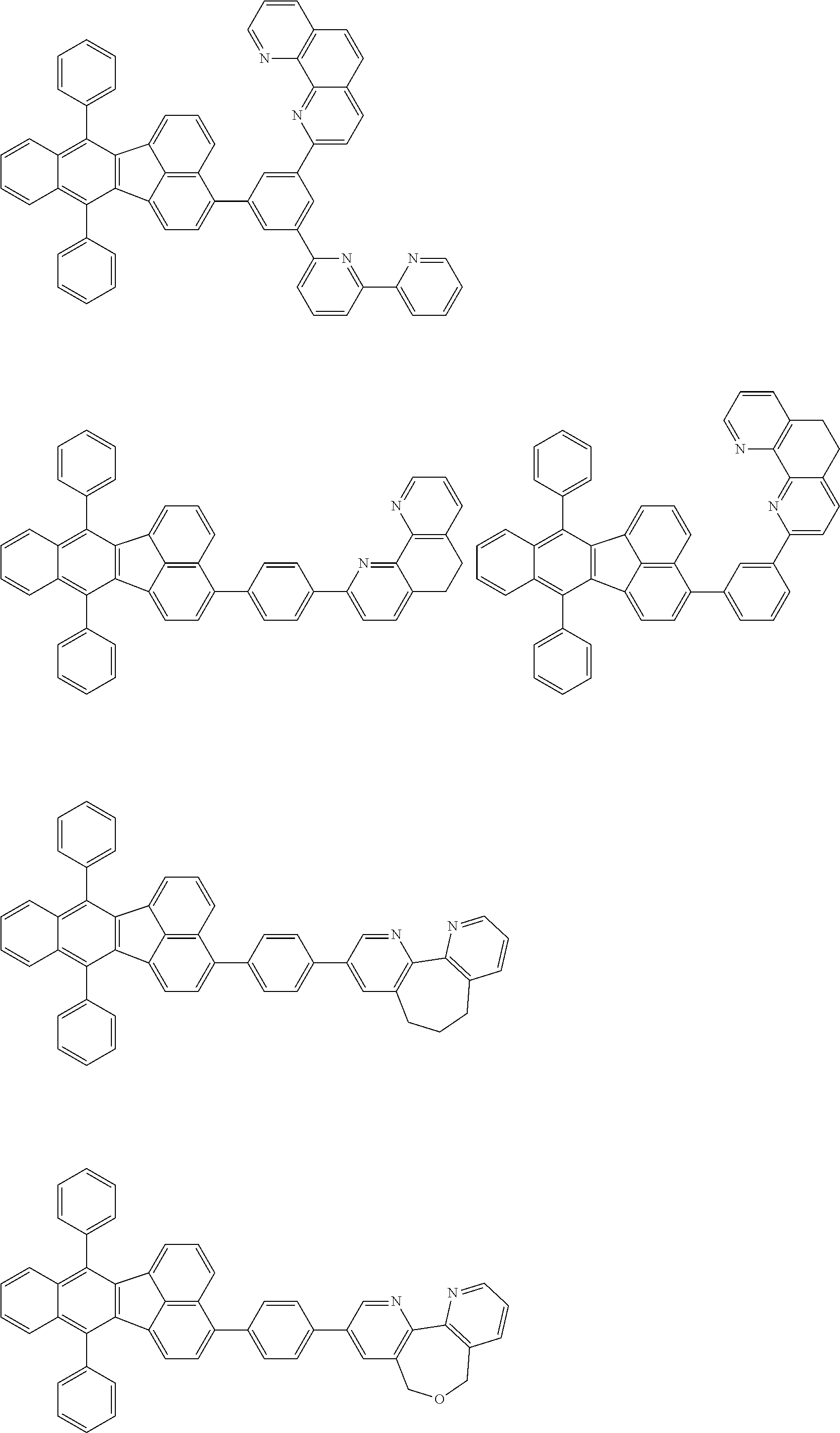

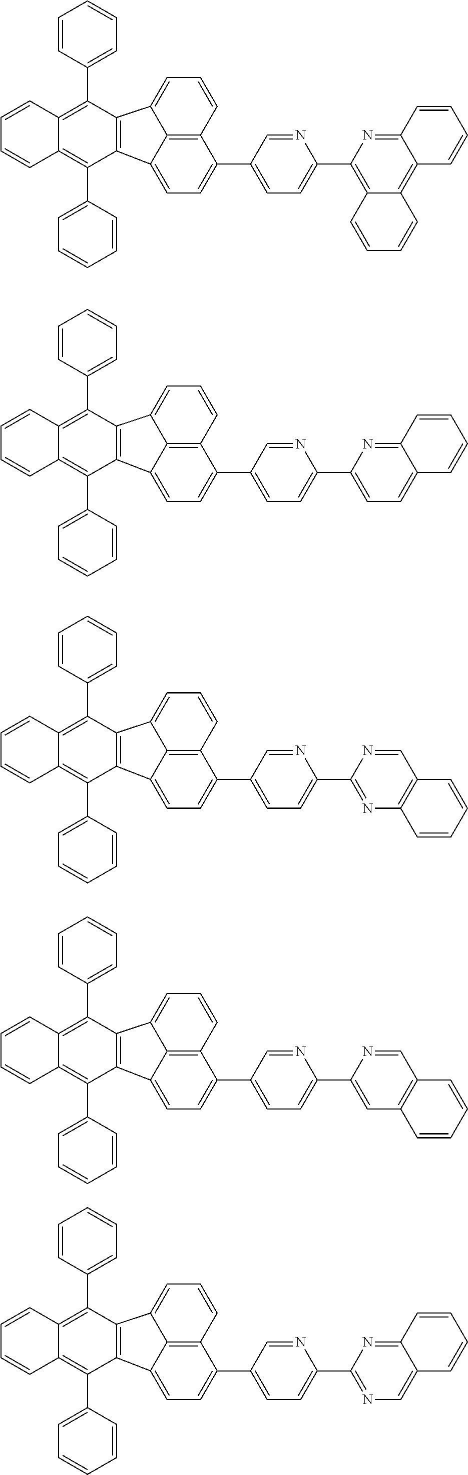

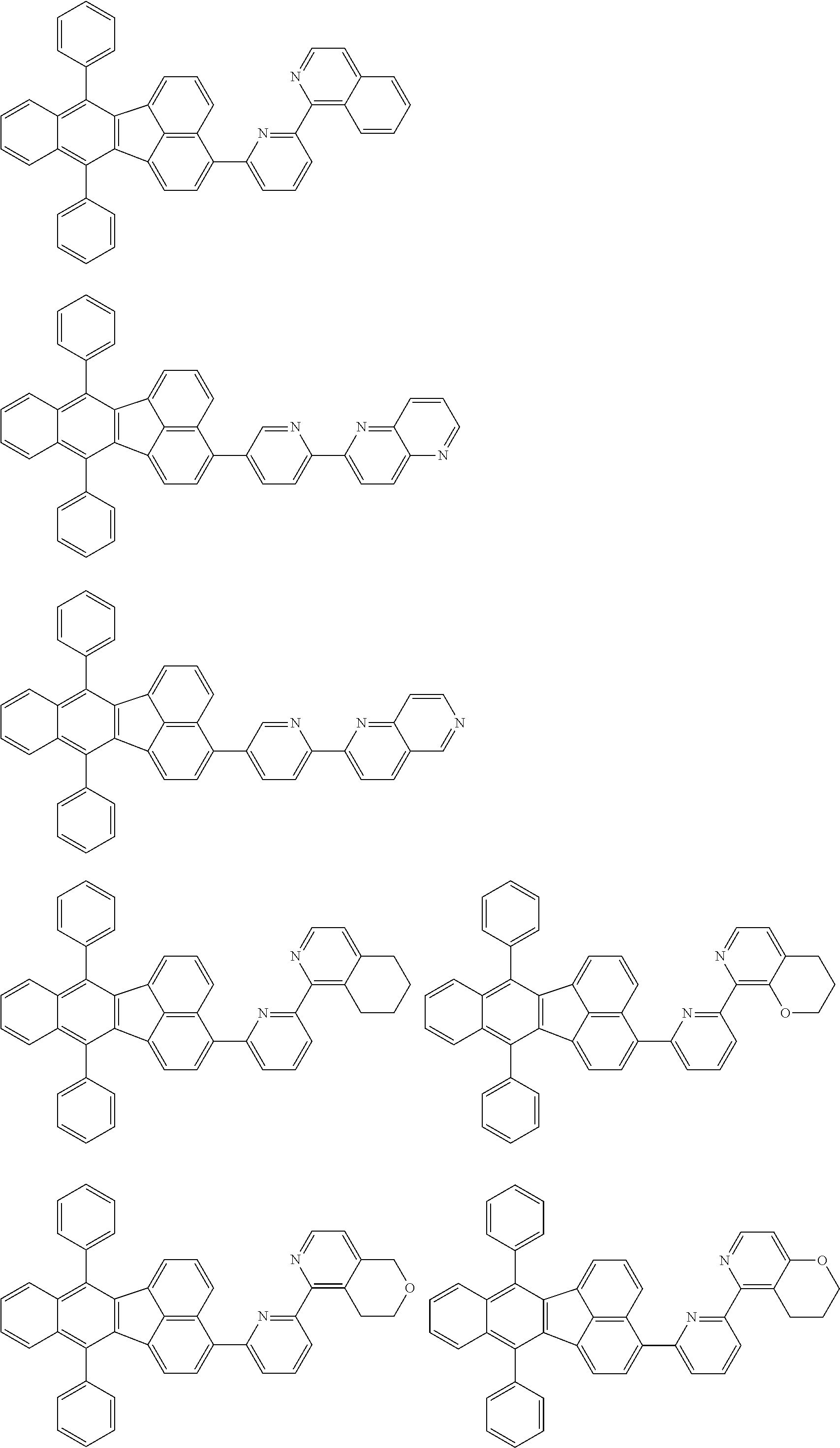

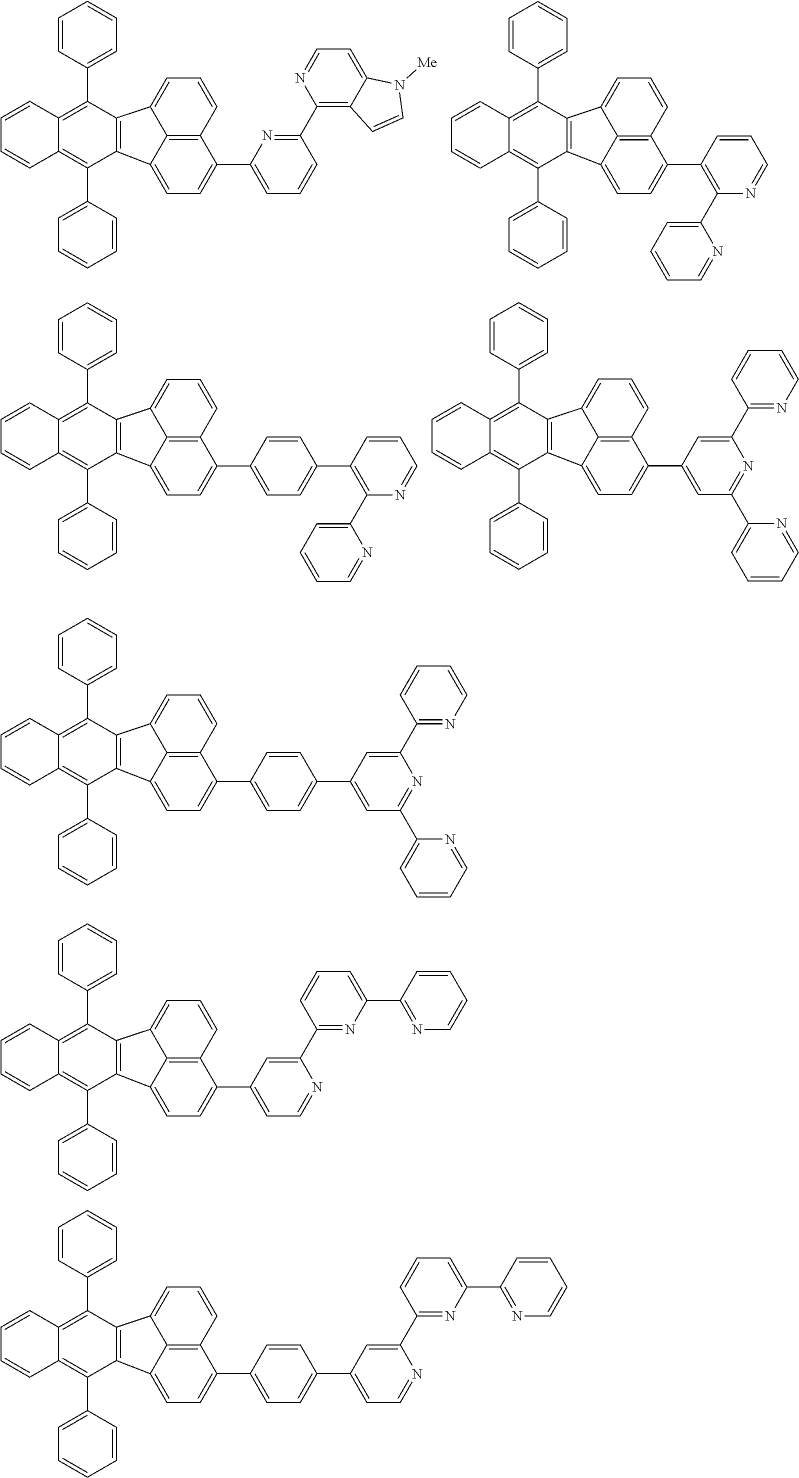

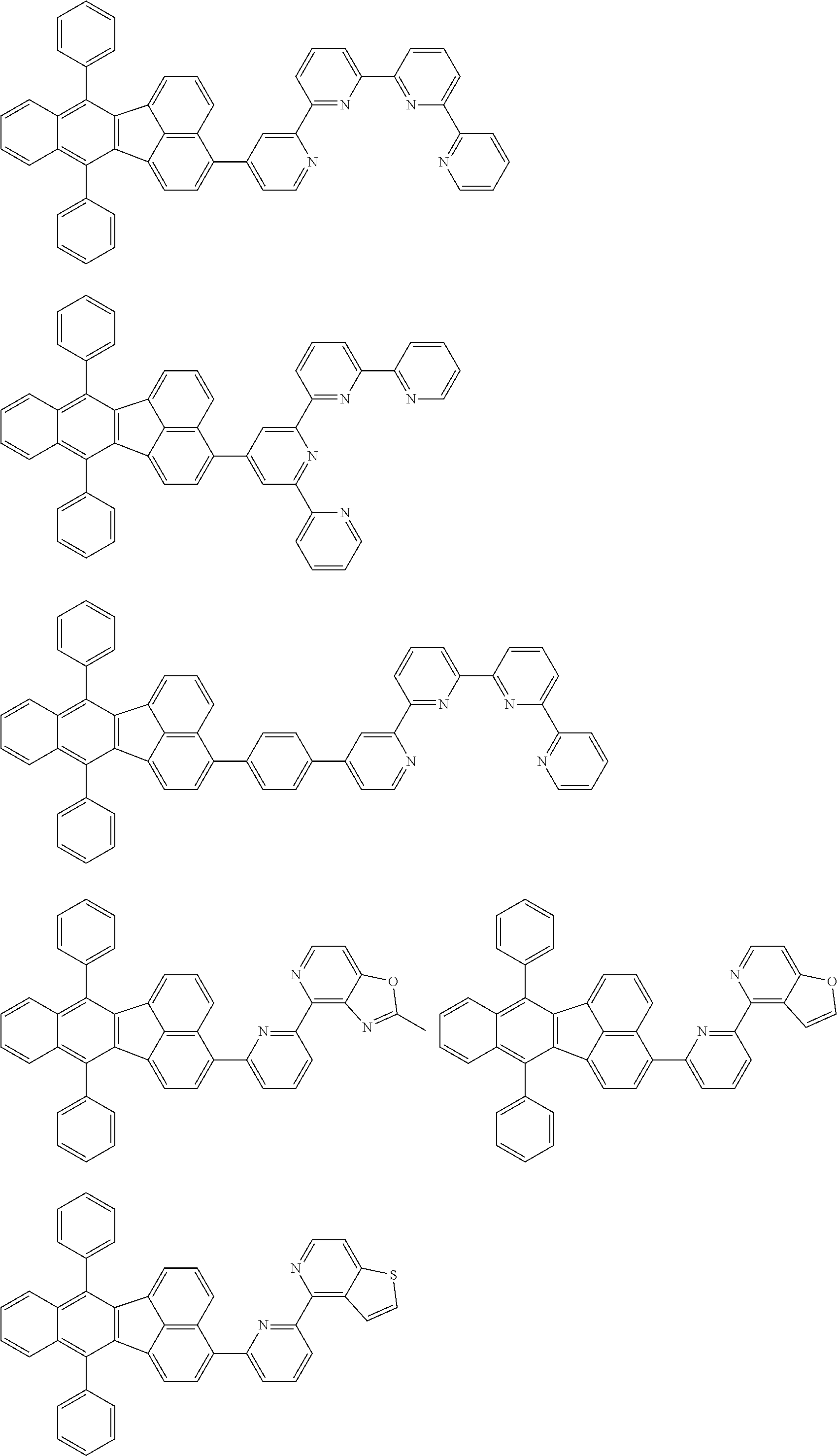

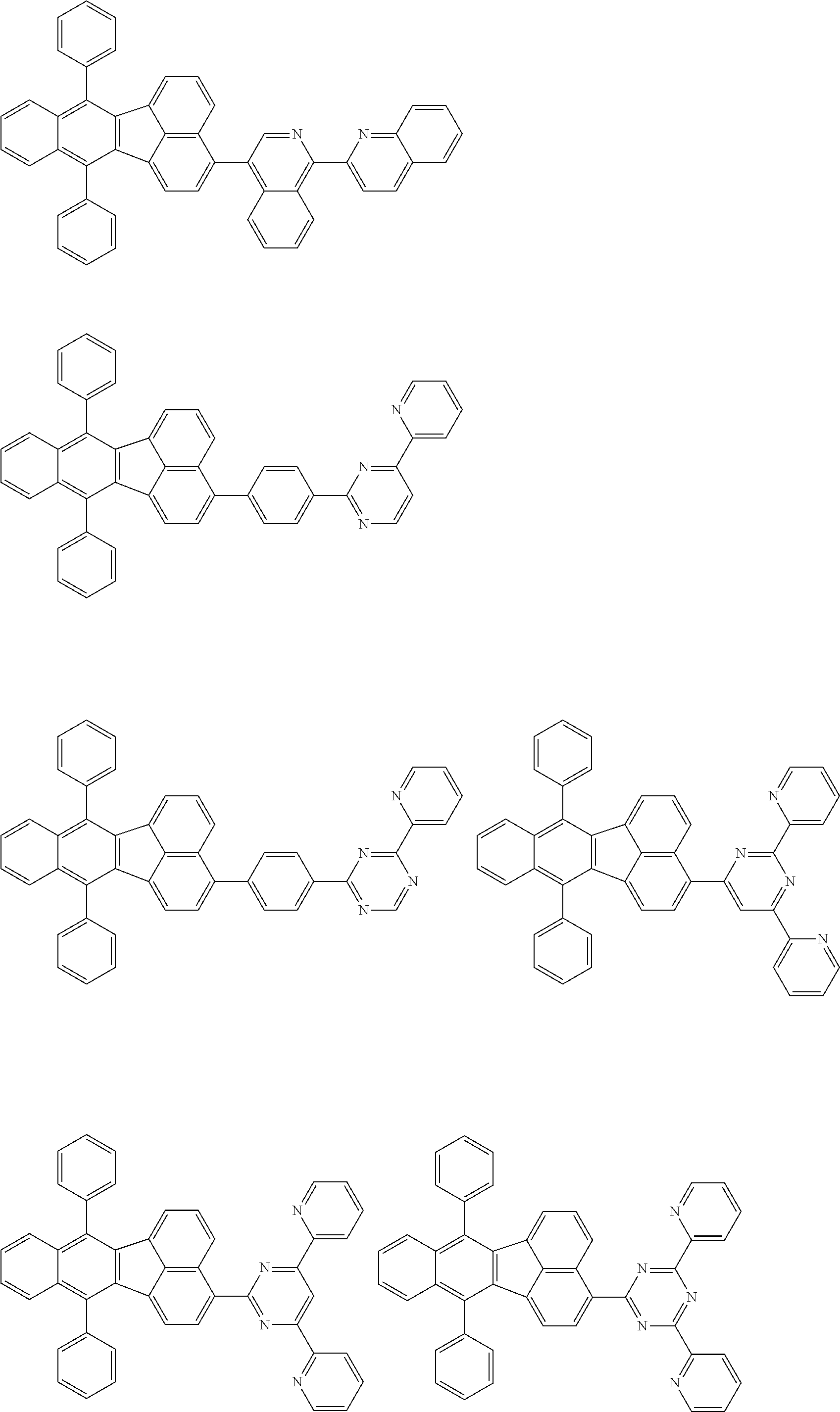

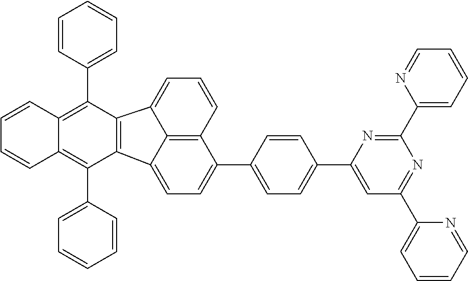 [SMILES: c1ccc(-c2c3c(c(-c4ccccc4)c4ccccc24)-c2ccc(-c4ccc(-c5cc(-c6ccccn6)nc(-c6ccccn6)n5)cc4)c4cccc-3c24)cc1]

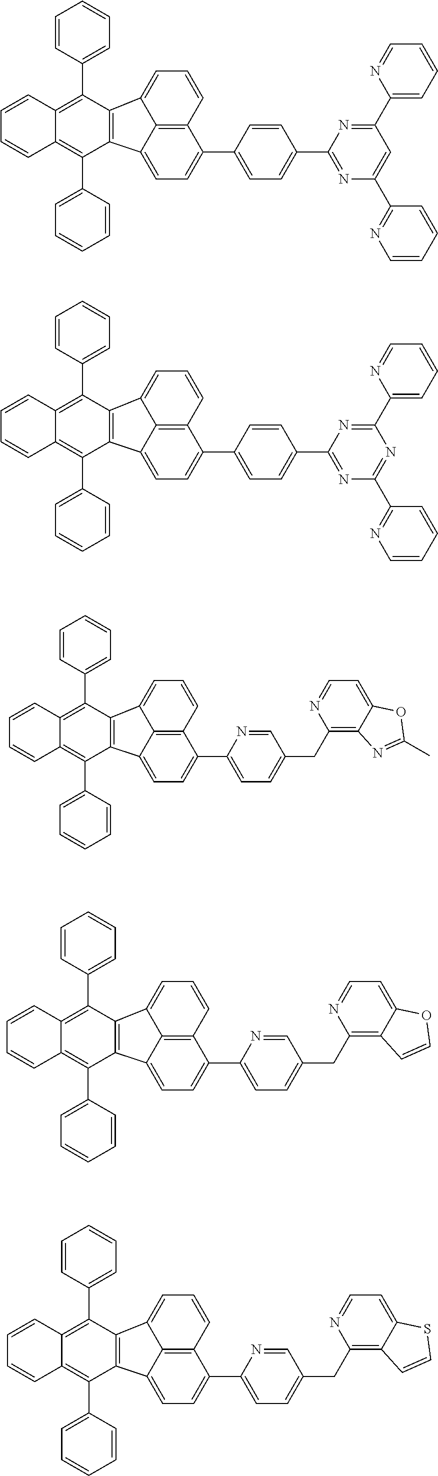 [SMILES: c1ccc(-c2c3c(c(-c4ccccc4)c4ccccc24)-c2ccc(-c4ccc(-c5nc(-c6ccccn6)cc(-c6ccccn6)n5)cc4)c4cccc-3c24)cc1 ; c1ccc(-c2c3c(c(-c4ccccc4)c4ccccc24)-c2ccc(-c4ccc(-c5nc(-c6ccccn6)nc(-c6ccccn6)n5)cc4)c4cccc-3c24)cc1 ; Cc1nc2c(Cc3ccc(-c4ccc5c6c(cccc46)-c4c-5c(-c5ccccc5)c5ccccc5c4-c4ccccc4)nc3)nccc2o1 ; c1ccc(-c2c3c(c(-c4ccccc4)c4ccccc24)-c2ccc(-c4ccc(Cc5nccc6occc56)cn4)c4cccc-3c24)cc1 ; c1ccc(-c2c3c(c(-c4ccccc4)c4ccccc24)-c2ccc(-c4ccc(Cc5nccc6sccc56)cn4)c4cccc-3c24)cc1]

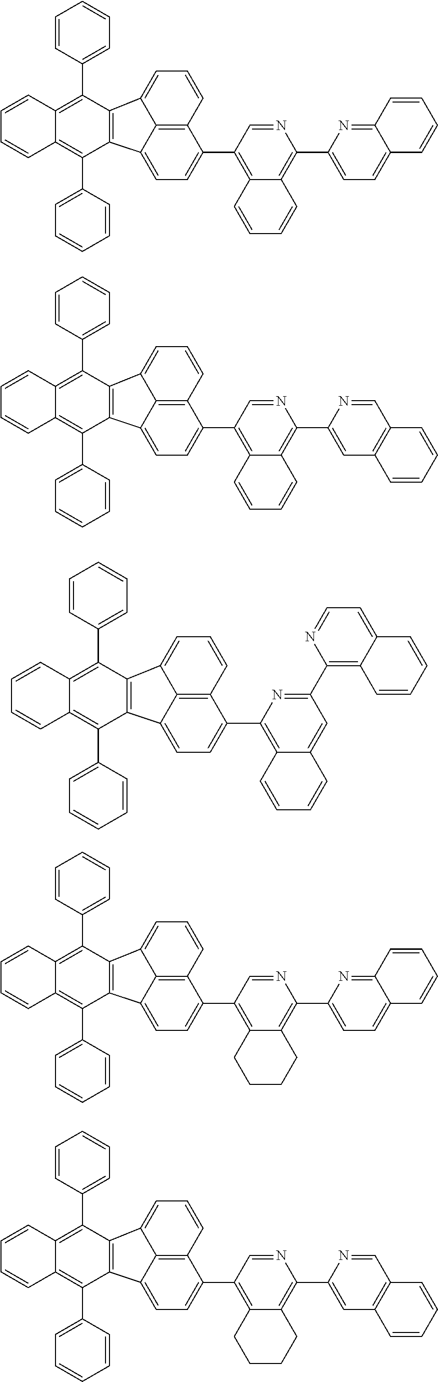

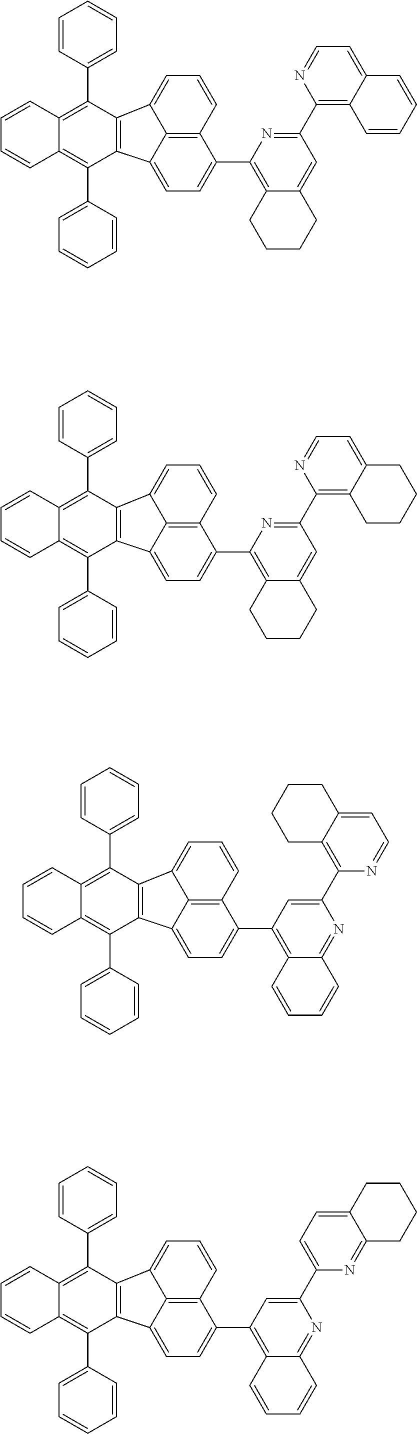 [SMILES: c1ccc(-c2c3c(c(-c4ccccc4)c4ccccc24)-c2ccc(-c4nc(-c5nccc6ccccc56)cc5c4CCCC5)c4cccc-3c24)cc1 ; c1ccc(-c2c3c(c(-c4ccccc4)c4ccccc24)-c2ccc(-c4nc(-c5nccc6c5CCCC6)cc5c4CCCC5)c4cccc-3c24)cc1 ; c1ccc(-c2c3c(c(-c4ccccc4)c4ccccc24)-c2ccc(-c4cc(-c5nccc6c5CCCC6)nc5ccccc45)c4cccc-3c24)cc1 ; c1ccc(-c2c3c(c(-c4ccccc4)c4ccccc24)-c2ccc(-c4cc(-c5ccc6c(n5)CCCC6)nc5ccccc45)c4cccc-3c24)cc1]

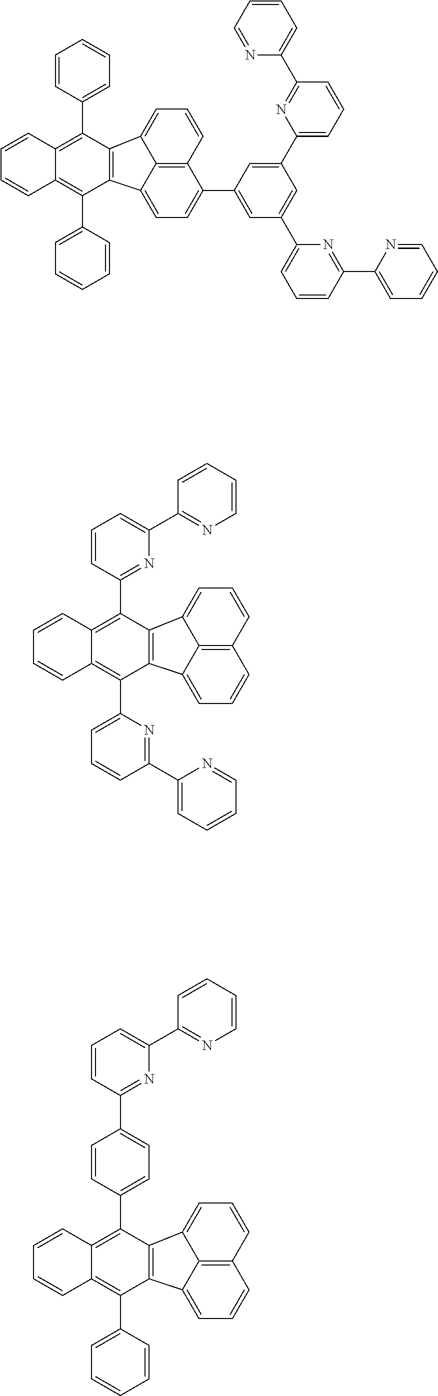 [SMILES: c1ccc(-c2c3c(c(-c4ccccc4)c4ccccc24)-c2ccc(-c4cc(-c5cccc(-c6ccccn6)n5)cc(-c5cccc(-c6ccccn6)n5)c4)c4cccc-3c24)cc1 ; c1ccc(-c2cccc(-c3c4c(c(-c5cccc(-c6ccccn6)n5)c5ccccc35)-c3cccc5cccc-4c35)n2)nc1 ; c1ccc(-c2c3c(c(-c4ccc(-c5cccc(-c6ccccn6)n5)cc4)c4ccccc24)-c2cccc4cccc-3c24)cc1]

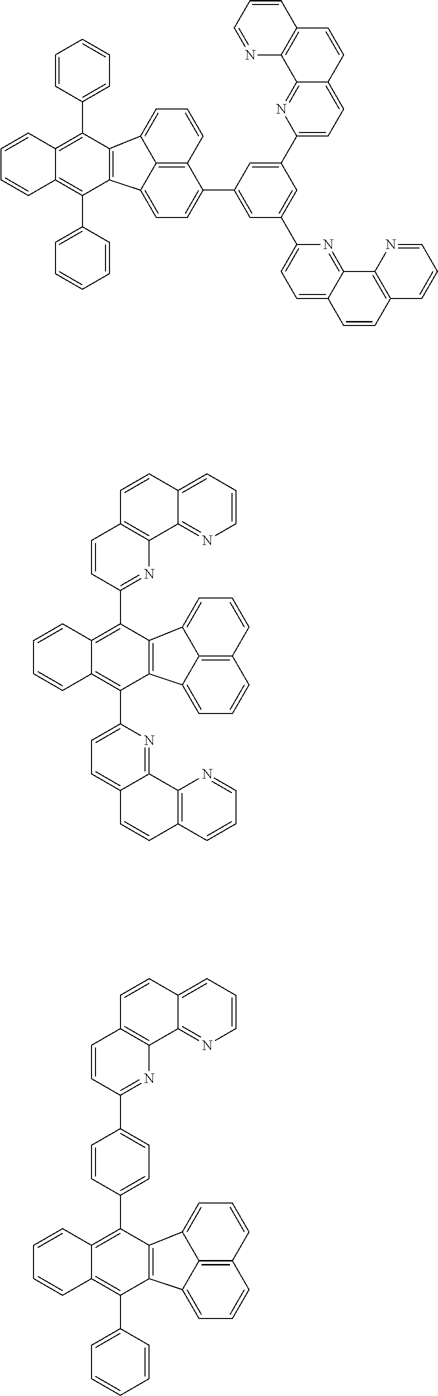 [SMILES: c1ccc(-c2c3c(c(-c4ccccc4)c4ccccc24)-c2ccc(-c4cc(-c5ccc6ccc7cccnc7c6n5)cc(-c5ccc6ccc7cccnc7c6n5)c4)c4cccc-3c24)cc1 ; c1cc2c3c(cccc3c1)-c1c-2c(-c2ccc3ccc4cccnc4c3n2)c2ccccc2c1-c1ccc2ccc3cccnc3c2n1 ; c1ccc(-c2c3c(c(-c4ccc(-c5ccc6ccc7cccnc7c6n5)cc4)c4ccccc24)-c2cccc4cccc-3c24)cc1]

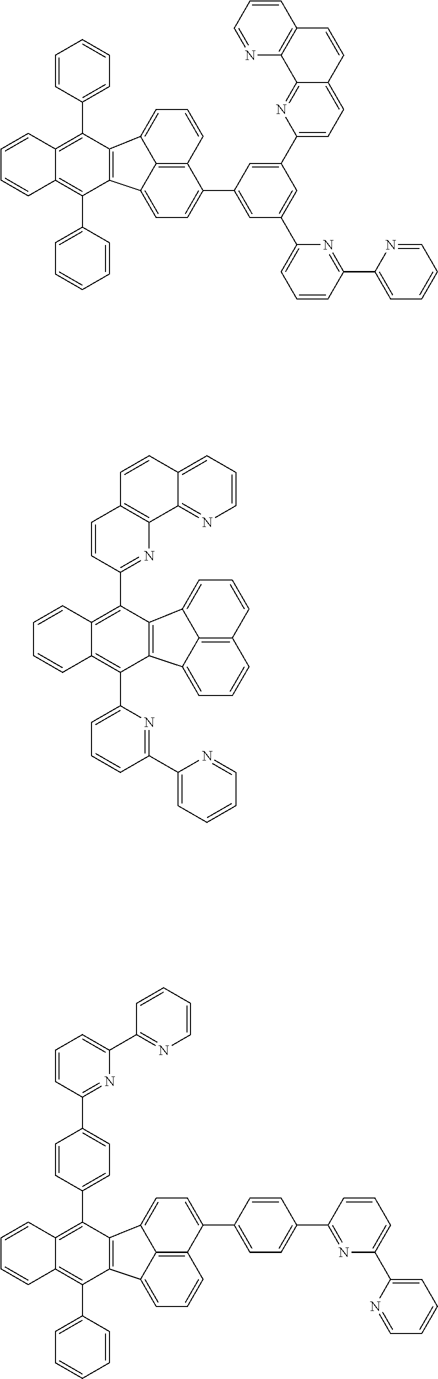 [SMILES: c1ccc(-c2c3c(c(-c4ccccc4)c4ccccc24)-c2ccc(-c4cc(-c5cccc(-c6ccccn6)n5)cc(-c5ccc6ccc7cccnc7c6n5)c4)c4cccc-3c24)cc1 ; c1ccc(-c2cccc(-c3c4c(c(-c5ccc6ccc7cccnc7c6n5)c5ccccc35)-c3cccc5cccc-4c35)n2)nc1 ; c1ccc(-c2c3c(c(-c4ccc(-c5cccc(-c6ccccn6)n5)cc4)c4ccccc24)-c2ccc(-c4ccc(-c5cccc(-c6ccccn6)n5)cc4)c4cccc-3c24)cc1]

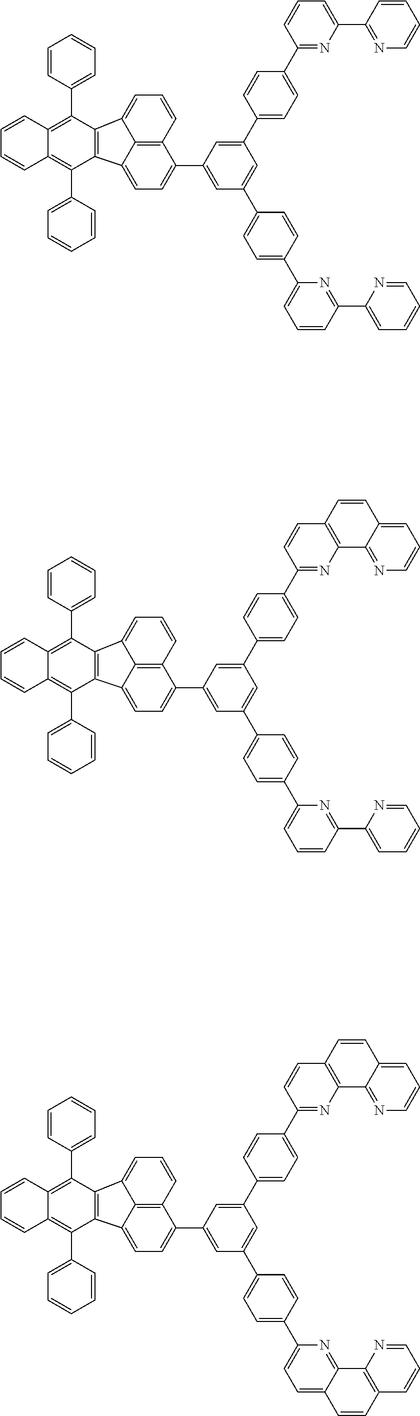 [SMILES: c1ccc(-c2c3c(c(-c4ccccc4)c4ccccc24)-c2ccc(-c4cc(-c5ccc(-c6cccc(-c7ccccn7)n6)cc5)cc(-c5ccc(-c6cccc(-c7ccccn7)n6)cc5)c4)c4cccc-3c24)cc1 ; c1ccc(-c2c3c(c(-c4ccccc4)c4ccccc24)-c2ccc(-c4cc(-c5ccc(-c6cccc(-c7ccccn7)n6)cc5)cc(-c5ccc(-c6ccc7ccc8cccnc8c7n6)cc5)c4)c4cccc-3c24)cc1 ; c1ccc(-c2c3c(c(-c4ccccc4)c4ccccc24)-c2ccc(-c4cc(-c5ccc(-c6ccc7ccc8cccnc8c7n6)cc5)cc(-c5ccc(-c6ccc7ccc8cccnc8c7n6)cc5)c4)c4cccc-3c24)cc1]

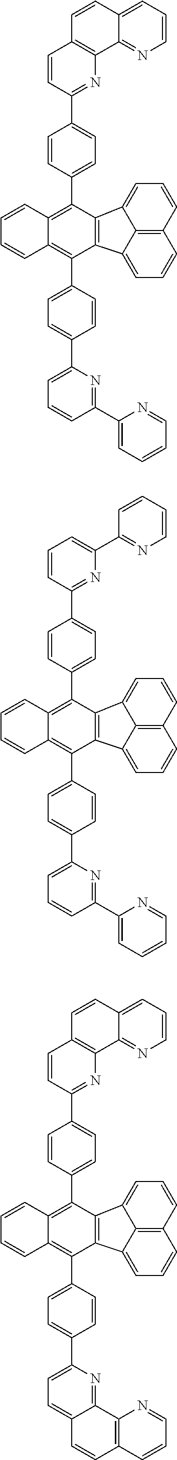

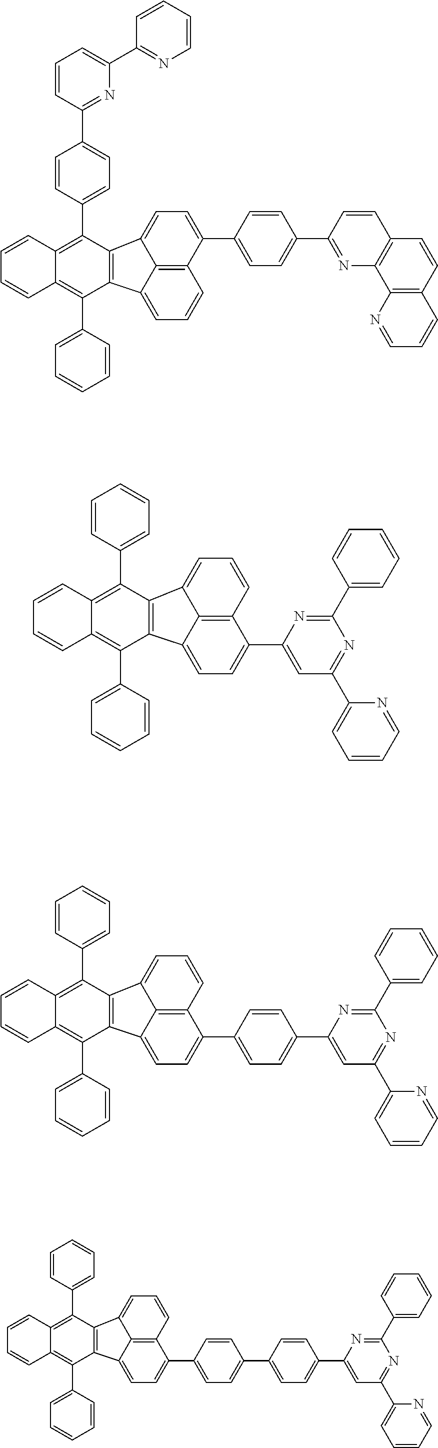 [SMILES: c1ccc(-c2c3c(c(-c4ccc(-c5cccc(-c6ccccn6)n5)cc4)c4ccccc24)-c2ccc(-c4ccc(-c5ccc6ccc7cccnc7c6n5)cc4)c4cccc-3c24)cc1 ; c1ccc(-c2nc(-c3ccccn3)cc(-c3ccc4c5c(cccc35)-c3c-4c(-c4ccccc4)c4ccccc4c3-c3ccccc3)n2)cc1 ; c1ccc(-c2nc(-c3ccc(-c4ccc5c6c(cccc46)-c4c-5c(-c5ccccc5)c5ccccc5c4-c4ccccc4)cc3)cc(-c3ccccn3)n2)cc1 ; c1ccc(-c2nc(-c3ccc(-c4ccc(-c5ccc6c7c(cccc57)-c5c-6c(-c6ccccc6)c6ccccc6c5-c5ccccc5)cc4)cc3)cc(-c3ccccn3)n2)cc1]

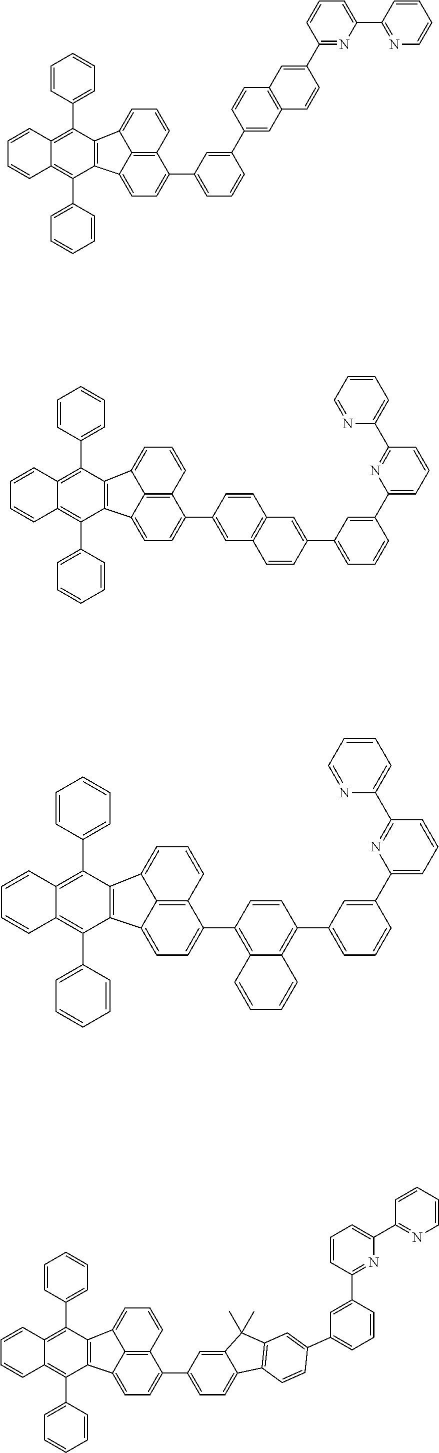 [SMILES: c1ccc(-c2c3c(c(-c4ccccc4)c4ccccc24)-c2ccc(-c4cccc(-c5ccc6cc(-c7cccc(-c8ccccn8)n7)ccc6c5)c4)c4cccc-3c24)cc1 ; c1ccc(-c2c3c(c(-c4ccccc4)c4ccccc24)-c2ccc(-c4ccc5cc(-c6cccc(-c7cccc(-c8ccccn8)n7)c6)ccc5c4)c4cccc-3c24)cc1 ; c1ccc(-c2c3c(c(-c4ccccc4)c4ccccc24)-c2ccc(-c4ccc(-c5cccc(-c6cccc(-c7ccccn7)n6)c5)c5ccccc45)c4cccc-3c24)cc1 ; CC1(C)c2cc(-c3cccc(-c4cccc(-c5ccccn5)n4)c3)ccc2-c2ccc(-c3ccc4c5c(cccc35)-c3c-4c(-c4ccccc4)c4ccccc4c3-c3ccccc3)cc21]

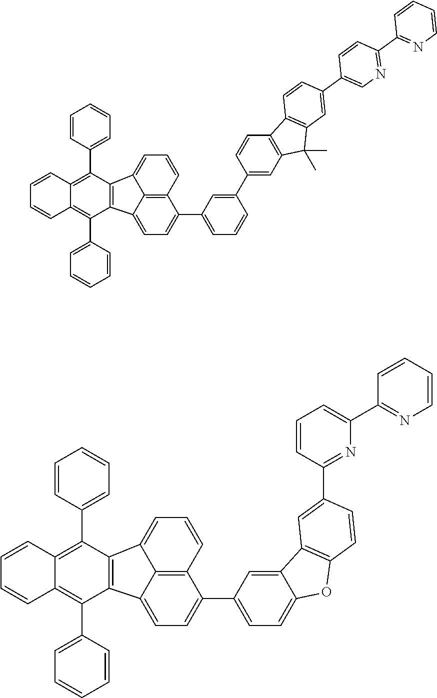 [SMILES: CC1(C)c2cc(-c3ccc(-c4ccccn4)nc3)ccc2-c2ccc(-c3cccc(-c4ccc5c6c(cccc46)-c4c-5c(-c5ccccc5)c5ccccc5c4-c4ccccc4)c3)cc21 ; c1ccc(-c2c3c(c(-c4ccccc4)c4ccccc24)-c2ccc(-c4ccc5oc6ccc(-c7cccc(-c8ccccn8)n7)cc6c5c4)c4cccc-3c24)cc1]

### (Other Constitution of Electron-Transporting Region)

As a material other than those which can used in the electron-transporting region, especially as the electron-transporting layer, 1) a metal complex such as a beryllium complex and a zinc complex; 2) a heteroaromatic compound such as an imidazole derivative, a benzimidazole derivative, an azine derivative, carbazole derivative and a phenanthroline derivative; and 3) a polymer compound; or the like can be mentioned. Further, especially as the electron-injecting layer, magnesium, an alkaline earth metal, a compound thereof, and the like can be used. These materials may be mixed and used in combination with the compounds of the formulas (1) to (4) or the rare earth element described above, or a layer consisting of these compounds may separately be provided.

The case where the third layer (hole-barrier layer) is provided in the electron-transporting region will be described. The hole-barrier layer is a layer which has a function of preventing leakage of holes from the emitting layer to the electron-transporting layer, and is usually a layer arranged nearest on the emitting layer side in the electron-transporting region. As a material used for the layer, materials adapted to the function of the layer can be used among the compounds described above for the electron-transporting layer.

When the fourth layer (second electron-transporting layer) is provided in the electron-transporting region, the compounds described above for the electron-transporting layer or the like can be used as materials of the layer.

### (Cathode)

For the cathode, metals, alloys, electrically conductive compounds, mixtures thereof, and the like, which have small work function (specifically 3.8 eV or less) are preferably used. Specific examples of such a cathode material include an element belonging to Group 1 or Group 2 of the Periodic Table of the Elements, i.e., an alkali metal such as lithium (Li) and cesium (Cs), and an alkaline earth metal such as magnesium (Mg), calcium (Ca) and strontium (Sr), and an alloy containing these (e.g., MgAg and AILi).

In the organic EL device according to an aspect of the present invention, the cathode is a layer which substantially does not include the rare earth element. The expression “substantially does not include” refers the case where the rare earth element is not included at all or the case where the rare earth element is unavoidably included as impurities.

### (Other Configuration of Organic EL Device)

Conventionally-known materials and device configurations can be applied to the organic EL device according to an aspect of the present invention as long as the electron-transporting region satisfy the above conditions, and further as long as the effects of the present invention are not impaired.

Device configurations, materials for forming each layer, and the like in the organic EL device according to an aspect of the present invention will be described below.

Examples of the device configuration of the organic EL device according to an aspect of the present invention include the following configurations:
(1) an anode/an emitting layer/an electron-transporting region/a cathode,
(2) an anode/a hole-transporting region/an emitting layer/an electron-transporting region/a cathode,
   wherein the hole-transporting region is usually composed of one or more layers selected from the hole-injecting layer and the hole-transporting layer.

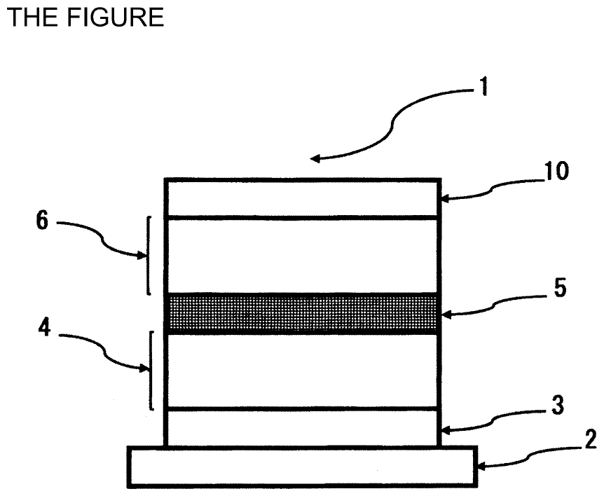

A schematic configuration of the organic EL device according to an aspect of the present invention will be described with reference to FIG. 1.

The organic EL device 1 according to an aspect of the present invention includes a substrate 2, an anode 3, an emitting layer 5, a cathode 10, a hole-transporting region 4 between the anode 3 and the emitting layer 5, and an electron-transporting region 6 between the emitting layer 5 and the cathode 10.

Each layer of the organic EL device will be described below.

### (Substrate)

The substrate is used as a support of an emitting device. As the substrate, glass, quartz, plastic or the like can be used, for example. Further, a flexible substrate may be used. The term “flexible substrate” means a bendable (flexible) substrate, and specific examples thereof include a plastic substrate formed of polycarbonate, polyvinyl chloride or the like.

### (Anode)

For the anode formed on the substrate, metals, alloys, electrically conductive compounds, mixtures thereof, and the like, which have large work function (specifically 4.0 eV or more) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), or the like.

### (Hole-Injecting Layer)

The hole-injecting layer is a layer containing a substance having high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, and the like), or the like can be given.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. Provided that a substance other than the above-described substances may be used as long as the substance has higher hole-transporting property than electron-transporting property. The layer containing the substance having high hole-transporting property may be not only a single layer, but also layers in which two or more layers formed of the above-described substances are stacked.

### (Guest (Dopant) Material of Emitting Layer)

The emitting layer is a layer containing a substance having high luminous property, and various materials can be used. For example, as the substance having high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes and platinum complexes are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes and europium complexes are used.

### (Host Material for Emitting Layer)

The emitting layer may have a constitution in which the substance having high emitting property (guest material) is dispersed in another substance (host material). As a substance for dispersing the substance having high emitting property, a variety of substances can be used, and it is preferable to use a substance having a higher lowest unoccupied molecular orbital level (LUMO level) and a lower highest occupied molecular orbital level (HOMO level) than a substance having high emitting property.

As a substance (host material) for dispersing the substance having high emitting property, 1) a metal complex such as an aluminum complex, a beryllium complex, and a zinc complex, 2) a heterocyclic compound such as an oxadiazole derivative, a benzimidazole derivative, and a phenanthroline derivative, 3) a fused aromatic compound such as a carbazole derivative, an anthracene derivative, a phenanthrene derivative, a pyrene derivative, and a chrysene derivative, and 4) an aromatic amine compound such as a triarylamine derivative and a fused polycyclic aromatic amine derivative are used.

A compound having delayed fluorescence (thermally activated delayed fluorescence) can also be used as the host material. It is also preferable that the emitting layer includes the material used in the present invention described above and the host compound having delayed fluorescence.

### (Hole-Blocking Layer, Exciton-Blocking Layer)

A hole-blocking layer, an exciton (triplet)-blocking layer, and the like may be provided adjacent to the emitting layer. The hole-blocking layer is a layer which has a function of preventing leakage of holes from the emitting layer to the electron-transporting layer. The exciton-blocking layer is a layer which has a function of preventing diffusion of excitons generated in the emitting layer into the adjacent layers to confine the excitons within the emitting layer.

In the organic EL device according to an aspect of the present invention, the thickness of each layer is not particularly limited, but is normally preferable several nm to 1 μm generally in order to suppress defects such as pinholes, to suppress applied voltages to be low, and to improve luminous efficiency.

In the organic EL device according to an aspect of the present invention, the method for forming each layer is not particularly limited. A conventionally-known method for forming each layer such as a vacuum deposition process and a spin coating process can be used. Each layer such as the emitting layer can be formed by a known method such as a vacuum deposition process, a molecular beam deposition process (MBE process), or an application process such as a dipping process, a spin coating process, a casting process, a bar coating process and a roll coating process, using a solution prepared by dissolving the material in a solvent.

### [Electronic Apparatus]

An electronic apparatus according to an aspect of the present invention is characterized by including the organic EL device according to an aspect of the present invention.

Specific examples of the electronic apparatus include display components such as an organic EL panel module; display devices for a television, a cellular phone and a personal computer; and emitting devices such as a light and a vehicular lamp; and the like.

### EXAMPLES

### <Compound>

Compounds represented by the formulas (1) to (4) used in Examples 1 to 25 are shown below.

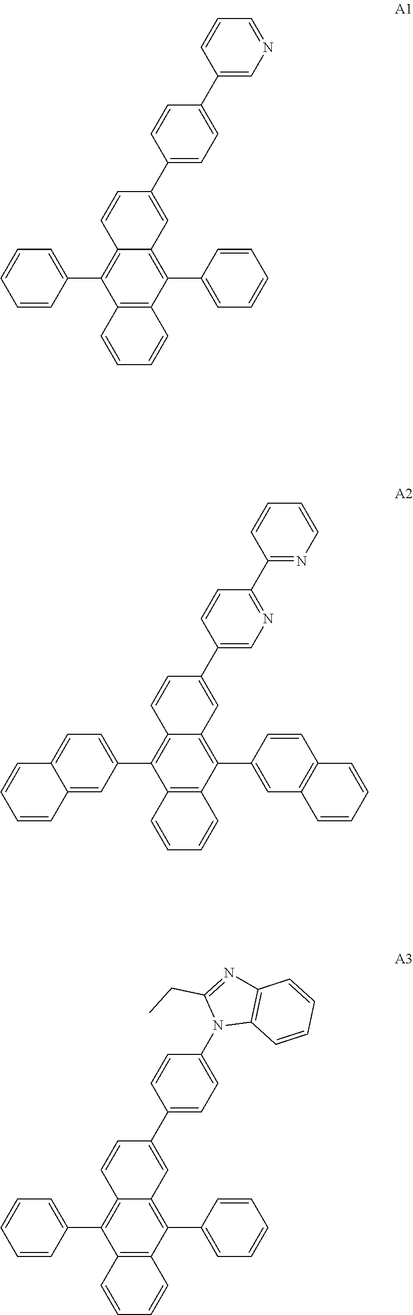 [SMILES: c1ccc(-c2c3ccccc3c(-c3ccccc3)c3cc(-c4ccc(-c5cccnc5)cc4)ccc23)cc1 ; c1ccc(-c2ccc(-c3ccc4c(-c5ccc6ccccc6c5)c5ccccc5c(-c5ccc6ccccc6c5)c4c3)cn2)nc1 ; CCc1nc2ccccc2n1-c1ccc(-c2ccc3c(-c4ccccc4)c4ccccc4c(-c4ccccc4)c3c2)cc1]

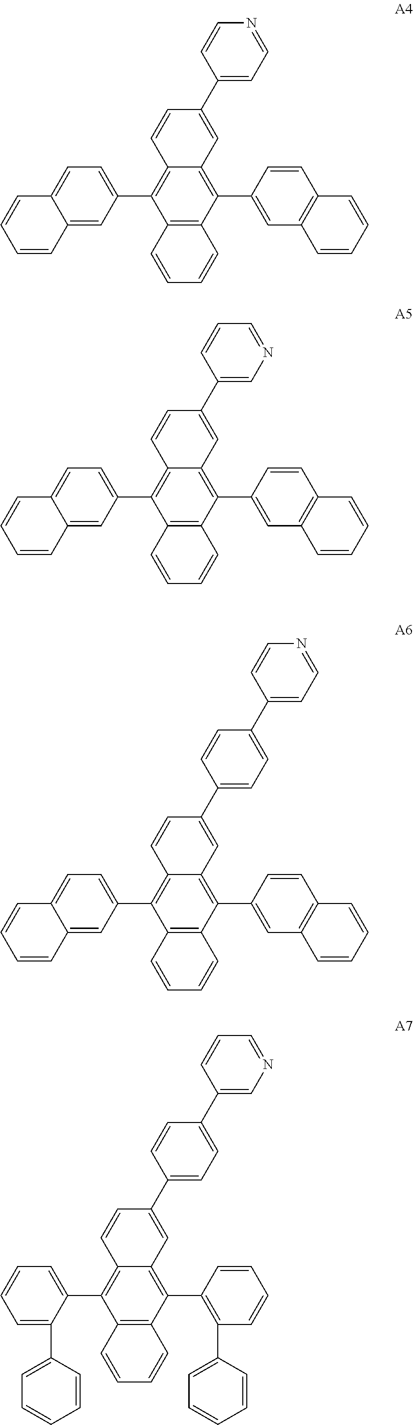

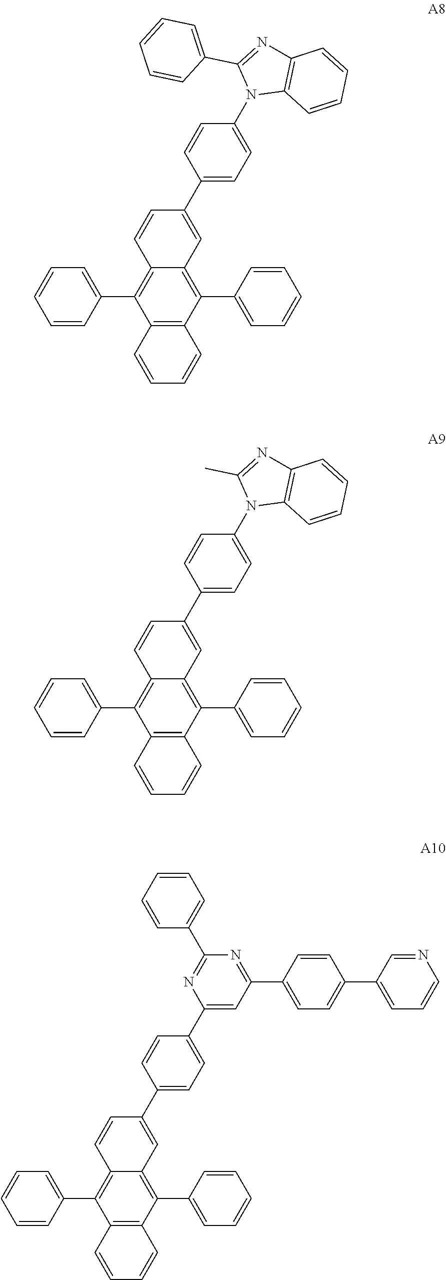 [SMILES: c1ccc(-c2c3ccccc3c(-c3ccccc3)c3cc(-c4ccc(-n5c(-c6ccccc6)nc6ccccc65)cc4)ccc23)cc1 ; Cc1nc2ccccc2n1-c1ccc(-c2ccc3c(-c4ccccc4)c4ccccc4c(-c4ccccc4)c3c2)cc1 ; c1ccc(-c2nc(-c3ccc(-c4cccnc4)cc3)cc(-c3ccc(-c4ccc5c(-c6ccccc6)c6ccccc6c(-c6ccccc6)c5c4)cc3)n2)cc1]

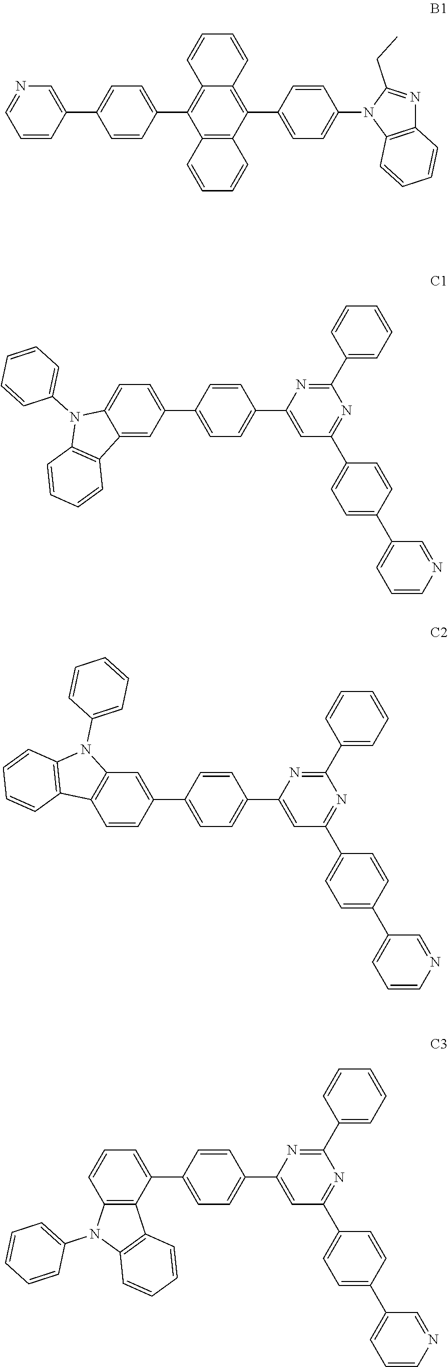 [SMILES: CCc1nc2ccccc2n1-c1ccc(-c2c3ccccc3c(-c3ccc(-c4cccnc4)cc3)c3ccccc23)cc1 ; c1ccc(-c2nc(-c3ccc(-c4cccnc4)cc3)cc(-c3ccc(-c4ccc5c(c4)c4ccccc4n5-c4ccccc4)cc3)n2)cc1 ; c1ccc(-c2nc(-c3ccc(-c4cccnc4)cc3)cc(-c3ccc(-c4ccc5c6ccccc6n(-c6ccccc6)c5c4)cc3)n2)cc1 ; c1ccc(-c2nc(-c3ccc(-c4cccnc4)cc3)cc(-c3ccc(-c4cccc5c4c4ccccc4n5-c4ccccc4)cc3)n2)cc1]

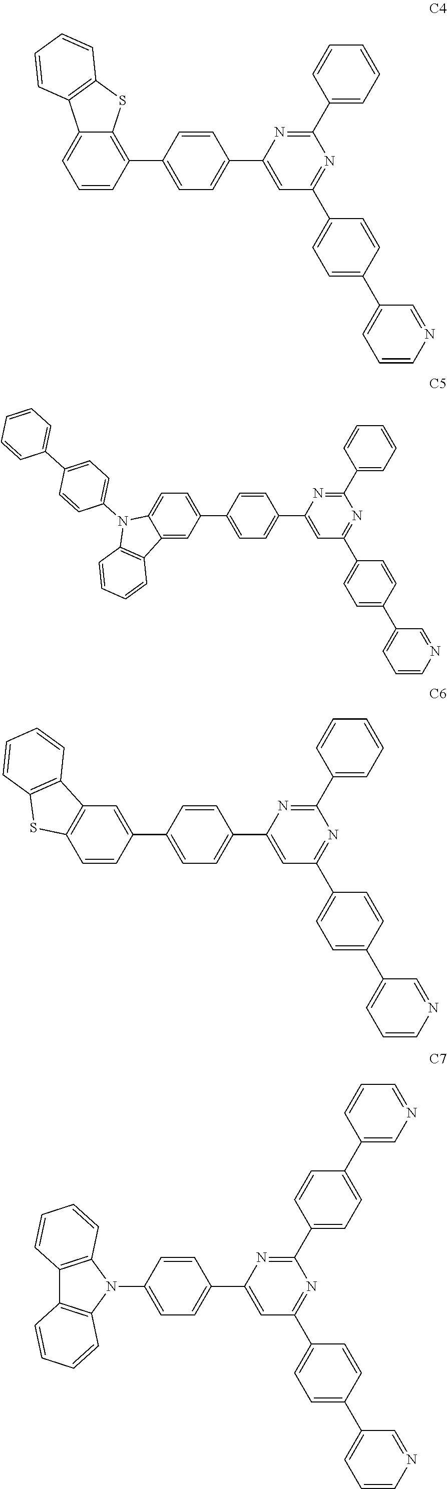 [SMILES: c1ccc(-c2nc(-c3ccc(-c4cccnc4)cc3)cc(-c3ccc(-c4cccc5c4sc4ccccc45)cc3)n2)cc1 ; c1ccc(-c2ccc(-n3c4ccccc4c4cc(-c5ccc(-c6cc(-c7ccc(-c8cccnc8)cc7)nc(-c7ccccc7)n6)cc5)ccc43)cc2)cc1 ; c1ccc(-c2nc(-c3ccc(-c4cccnc4)cc3)cc(-c3ccc(-c4ccc5sc6ccccc6c5c4)cc3)n2)cc1 ; c1cncc(-c2ccc(-c3cc(-c4ccc(-n5c6ccccc6c6ccccc65)cc4)nc(-c4ccc(-c5cccnc5)cc4)n3)cc2)c1]

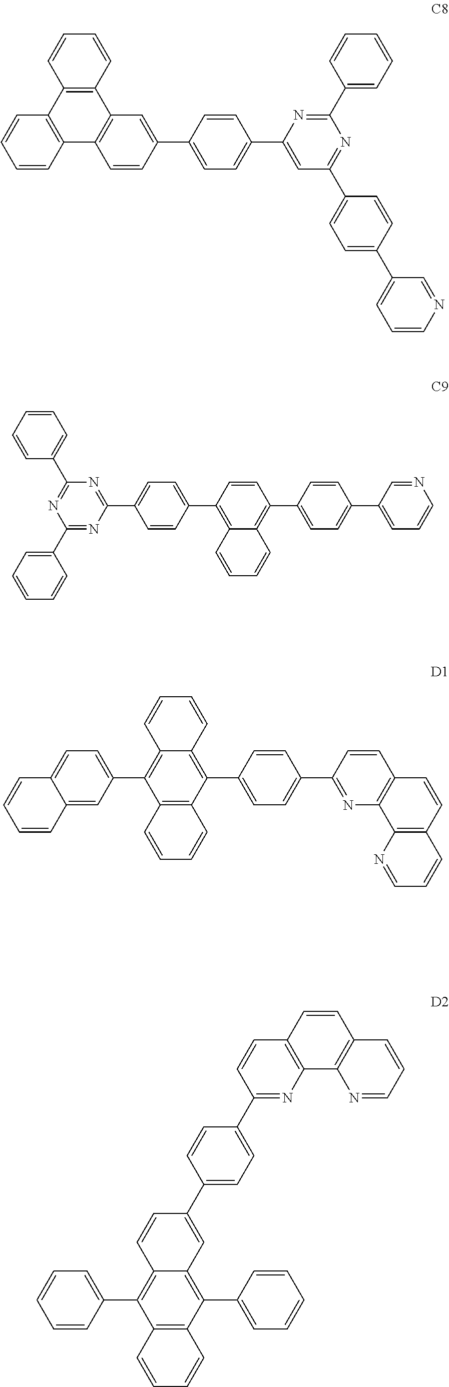 [SMILES: c1ccc(-c2nc(-c3ccc(-c4cccnc4)cc3)cc(-c3ccc(-c4ccc5c6ccccc6c6ccccc6c5c4)cc3)n2)cc1 ; c1ccc(-c2nc(-c3ccccc3)nc(-c3ccc(-c4ccc(-c5ccc(-c6cccnc6)cc5)c5ccccc45)cc3)n2)cc1 ; c1ccc2cc(-c3c4ccccc4c(-c4ccc(-c5ccc6ccc7cccnc7c6n5)cc4)c4ccccc34)ccc2c1 ; c1ccc(-c2c3ccccc3c(-c3ccccc3)c3cc(-c4ccc(-c5ccc6ccc7cccnc7c6n5)cc4)ccc23)cc1]

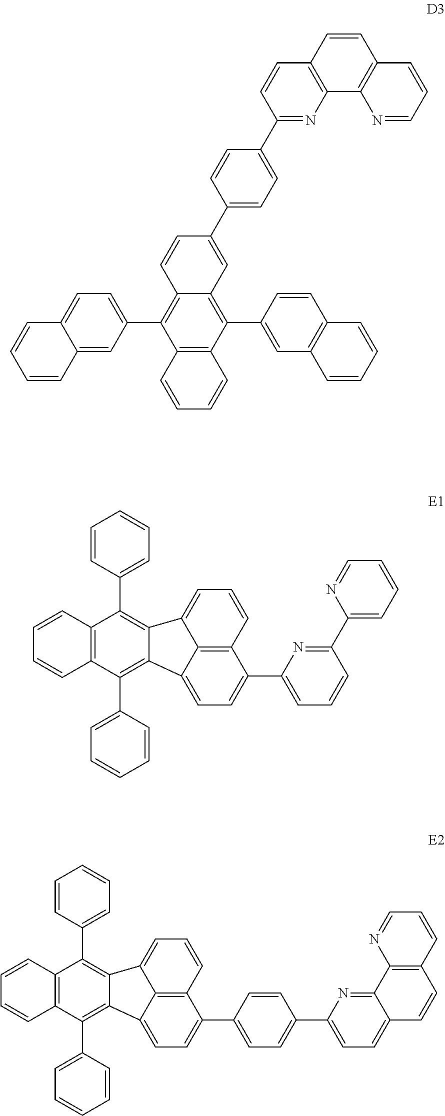 [SMILES: c1ccc2cc(-c3c4ccccc4c(-c4ccc5ccccc5c4)c4cc(-c5ccc(-c6ccc7ccc8cccnc8c7n6)cc5)ccc34)ccc2c1 ; c1ccc(-c2c3c(c(-c4ccccc4)c4ccccc24)-c2ccc(-c4cccc(-c5ccccn5)n4)c4cccc-3c24)cc1 ; c1ccc(-c2c3c(c(-c4ccccc4)c4ccccc24)-c2ccc(-c4ccc(-c5ccc6ccc7cccnc7c6n5)cc4)c4cccc-3c24)cc1]

Compound structures used in the fabrication of the organic EL devices of Comparative Examples 1 to 5 are shown below.

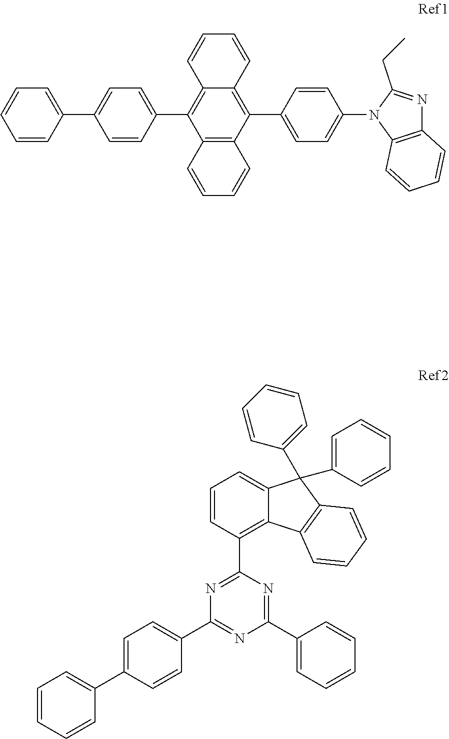 [SMILES: CCc1nc2ccccc2n1-c1ccc(-c2c3ccccc3c(-c3ccc(-c4ccccc4)cc3)c3ccccc23)cc1 ; c1ccc(-c2ccc(-c3nc(-c4ccccc4)nc(-c4cccc5c4-c4ccccc4C5(c4ccccc4)c4ccccc4)n3)cc2)cc1]

Other compound structures used in the fabrication of the organic EL devices of Examples 1 to 25 and Comparative Examples 1 to 5 are shown below.

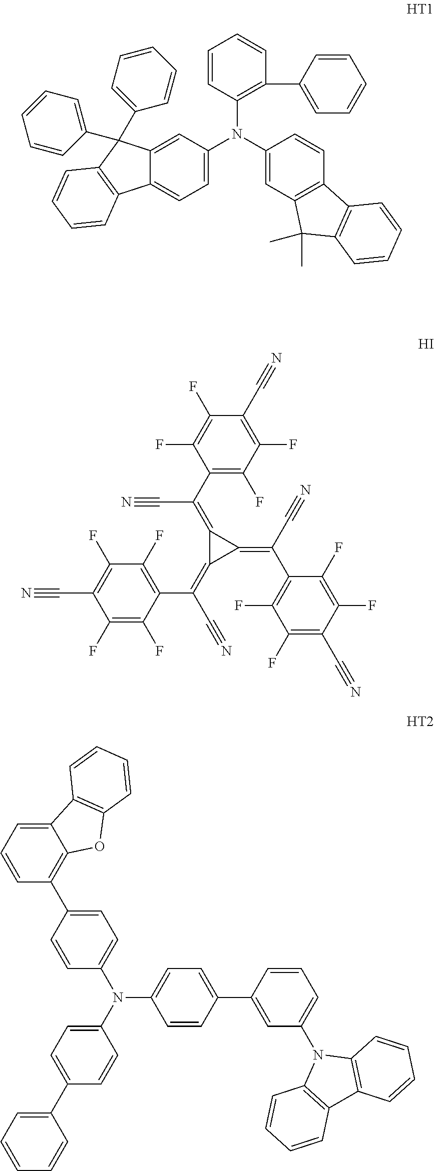 [SMILES: CC1(C)c2ccccc2-c2ccc(N(c3ccc4c(c3)C(c3ccccc3)(c3ccccc3)c3ccccc3-4)c3ccccc3-c3ccccc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

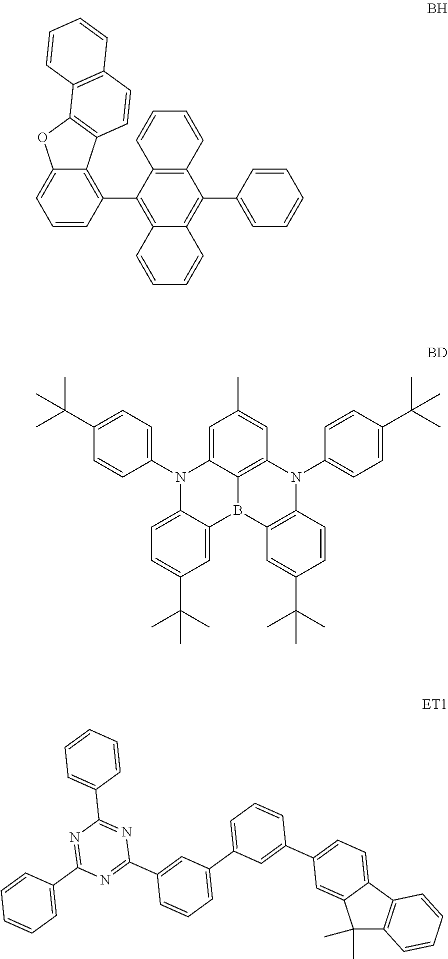 [SMILES: c1ccc(-c2c3ccccc3c(-c3cccc4oc5c6ccccc6ccc5c34)c3ccccc23)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; CC1(C)c2ccccc2-c2ccc(-c3cccc(-c4cccc(-c5nc(-c6ccccc6)nc(-c6ccccc6)n5)c4)c3)cc21]

### Example 1

### <Fabrication of Organic EL Device>

Organic EL devices were fabricated as follows.

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The ITO has the film thickness of 130 nm.

The glass substrate with the transparent electrode after being cleaned was mounted onto a substrate holder in a vacuum vapor deposition apparatus. First, compounds HT1 and HI were co-deposited on the surface on the side where the transparent electrode was formed so as to cover the transparent electrode to be 3% by mass in a proportion of the compound HI to form a first hole-transporting layer having the thickness of 10 nm.

The compound HT1 was deposited on the first hole-transporting layer to form a second hole-transporting layer having the thickness of 80 nm.

The compound HT2 was deposited on the second hole-transporting layer to form a third hole-transporting layer having the thickness of 5 nm.

A compound BH (host material) and a compound BD (dopant material) were co-deposited on the third hole-transporting layer to be 1% by mass in a proportion of the compound BD to form an emitting layer having the thickness of 20 nm.

A compound ET1 was deposited on the emitting layer to form a hole-barrier layer having the thickness of 5 nm.

A compound A1 was deposited on the hole-barrier layer to form a electron-transporting layer having the thickness of 25 nm.

A metal Yb was deposited on the electron-transporting layer to form an electron-injecting layer having the thickness of 1 nm.

A metal A1 was deposited on the electron-injecting layer to form a cathode having the thickness of 60 nm.

The device configuration of the organic EL device of Example 1 is schematically shown as follows.
HT1:HI(10:3%)/HT1 (80)/HT2(5)/BH:BD(20:1%)/ET1(5)/A1(25)/Yb(1)/AI(60)

The numerical values in parentheses indicate the film thickness (unit: nm). The numerical values represented by percent in parentheses indicate a proportion (% by mass) of the latter compound in the layer.

Further, the specific metal and specific compound are not used in the electron-transporting region, the electron-transporting region substantially does not include the specific metal and specific compound.

### <Evaluation of Organic EL Device>

The obtained organic EL device was evaluated as follows. The results are shown in Table 1.
Driving voltage

The initial property (driving voltage) of the organic EL device was measured by driving it using DC (direct current) constant current of 10 mA/cm² at room temperature. The numerical values in Table 1 are relative values when Comparative Example 1 described later is ±0 V.
Device lifetime

A voltage was applied to the obtained organic EL device at room temperature so that the current density became 50 mA/cm², and the time until the luminance became 95% of the initial luminance (LT95 (unit: h)) was measured. The numerical values in Table 1 are relative values when Comparative Example 1 described later is 100%.

### Examples 2 to 25 and Comparative Example 1

Organic EL devices were prepared and evaluated in the same manner as in Example 1, except that compounds shown in Table 1 were used as the material of the electron-transporting layer. The results are shown in Table 1.

The specific metal and specific compound are not used in the electron-transporting region, the electron-transporting region substantially does not include the specific metal and specific compound in Examples 2 to 25 and Comparative Example 1.

### Comparative Example 2

An organic EL device was fabricated and evaluated in the same manner as in Example 1, except that the compound Ref1 and Liq were co-deposited to be 1:1 in mass ratio to form the electron-transporting layer instead of the compound A1. The results are shown in Table 1.

### Comparative Example 3

An organic EL device was fabricated and evaluated in the same manner as in Example 1, except that the compound A1 and Liq were co-deposited to be 1:1 in mass ratio to form the electron-transporting layer by using Liq in addition to the compound A1. The results are shown in Table 1.

### Comparative Example 4

An organic EL device was fabricated and evaluated in the same manner as in Example 1, except that the compound Ref2 was used to form the electron-transporting layer instead of the compound A1. The results are shown in Table 1.

The specific metal and specific compound are not used in the electron-transporting region, the electron-transporting region substantially does not include the specific metal and specific compound in Comparative Example 4.

### Comparative Example 5

An organic EL device was fabricated and evaluated in the same manner as in Example 1, except that the compound Ref2 and Liq were co-deposited to be 1:1 in mass ratio to form the electron-transporting layer instead of the compound A1. The results are shown in Table 1.

TABLE 1

| | Electron-transporting | Driving | Device |
|---|---|---|---|
| | layer | voltage (V) | lifetime |
| Example 1 | Compound A1 | −0.70 | 667% |
| Example 2 | Compound A2 | −0.42 | 673% |
| Example 3 | Compound A3 | −0.66 | 733% |
| Example 4 | Compound A4 | −0.15 | 673% |
| Example 5 | Compound A5 | −0.52 | 653% |
| Example 6 | Compound A6 | −0.34 | 627% |
| Example 7 | Compound A7 | −0.62 | 647% |
| Example 8 | Compound A8 | −0.34 | 1040% |
| Example 9 | Compound A9 | −0.38 | 813% |
| Example 10 | Compound A10 | −0.46 | 900% |
| Example 11 | Compound B1 | −0.60 | 727% |
| Example 12 | Compound C1 | −0.42 | 867% |
| Example 13 | Compound C2 | −0.41 | 913% |
| Example 14 | Compound C3 | −0.47 | 860% |
| Example 15 | Compound C4 | −0.35 | 873% |
| Example 16 | Compound C5 | −0.25 | 720% |
| Example 17 | Compound C6 | −0.18 | 827% |
| Example 18 | Compound C7 | −0.36 | 993% |
| Example 19 | Compound C8 | −0.29 | 753% |
| Example 20 | Compound C9 | −0.65 | 1092% |
| Example 21 | Compound D1 | 0.10 | 660% |
| Example 22 | Compound D2 | −0.62 | 627% |
| Example 23 | Compound D3 | −0.55 | 693% |
| Example 24 | Compound E1 | −0.80 | 552% |
| Example 25 | Compound E2 | −0.80 | 564% |
| Comparative | Compound Ref1 | ±0 V | 100% |
| Example 1 | | (basing point) | (basing point) |
| Comparative | Compound Ref1 + | −0.36 | 574% |
| Example 2 | Liq (1:1) | | |
| Comparative | Compound A1 + | −0.82 | 601% |
| Example 3 | Liq (1:1) | | |
| Comparative | Compound Ref2 | 1.65 | 234% |
| Example 4 | | | |
| Comparative | Compound Ref2 + | −0.32 | 587% |
| Example 5 | Liq (1:1) | | |

As seen from Examples 1 to 25 being compared to Comparative Example 3, it was found that when the compounds having the specific structures represented by the formulas (1) to (4) were used for the electron-transporting region, approximately the same device performance thereof can be secured even without using Liq as compared to device performance in the case where Liq was used in combination with them, and the device lifetime thereof can remarkably be increased in some cases. On the other hand, as seen from Comparative Example 1 being compared to Comparative Example 2, it was found that when a compound other than specific compound was used for the electron-transporting region, the device lifetime thereof was extremely decreased without using Liq. As the case where Comparative Example 4 was compared to Comparative Example 5 was similar to it, it was found that the device performance even in terms of the voltage and even in terms of the device lifetime was extremely decreased without using Liq in combination with them.

### <Synthesis of Compound>

The compound C9 was synthesized through the synthetic route described below.

### (1) Synthesis of Intermediate 1

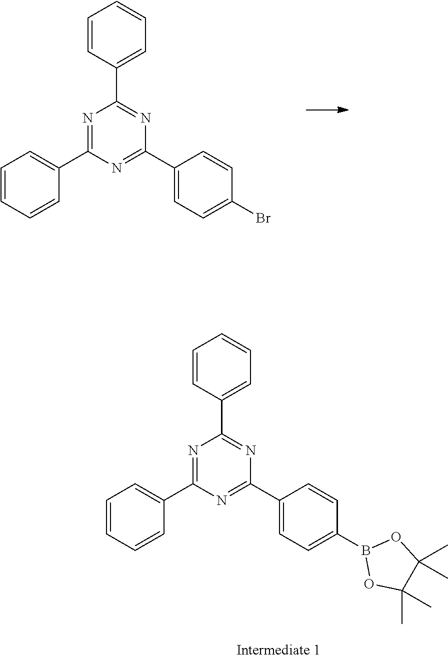 [SMILES: Brc1ccc(-c2nc(-c3ccccc3)nc(-c3ccccc3)n2)cc1 ; CC1(C)OB(c2ccc(-c3nc(-c4ccccc4)nc(-c4ccccc4)n3)cc2)OC1(C)C]

2-(4-bromophenyl)-4,6-diphenyl-1,3,5-triazine (40 g, 103 mmol), bis(pinacolato)diboron (65.4 g, 258 mmol), [1,1′-bis(diphenylphosphino)ferrocene]dichloropalladium (II)-dichloromethane adduct (4.2 g, 5.1 mmol), and potassium acetate (30.3 g, 309 mmol) were dissolved in 350 mL of N,N-dimethylformamide under a nitrogen atmosphere in a 1000 mL of three-necked round flask replaced by nitrogen-flashing. The reaction mixture was heated to 70° C. for two hours by using oil bath. It was cooled to room temperature, and then the reaction mixture was poured into water while stirring. The precipitated precipitate was collected by filtering. Subsequently, the precipitate was suspended in methanol (1 L), and then it was stirred at room temperature for two hours. The precipitate was collected by filtering again, and was dried. Thereafter, the crude product was dissolved in dichloromethane, it was filtered by using silica pad, and then it was washed with dichloromethane. Dichloromethane was evaporated under reduced pressure to obtain 40.6 g of white solid (91% yield), and it was used without further purification. Intermediate 1 was identified by using ESI-MS (electrospray ionization mass spectrometry). The molecular weight (M) calculated from the formula C₂₇H₂₆BN₃O₂ of the Intermediate 1 is 453, and a mass measurement value (M+1) obtained using the ESI-MS was 454.

### (2) Synthesis of Intermediate 2

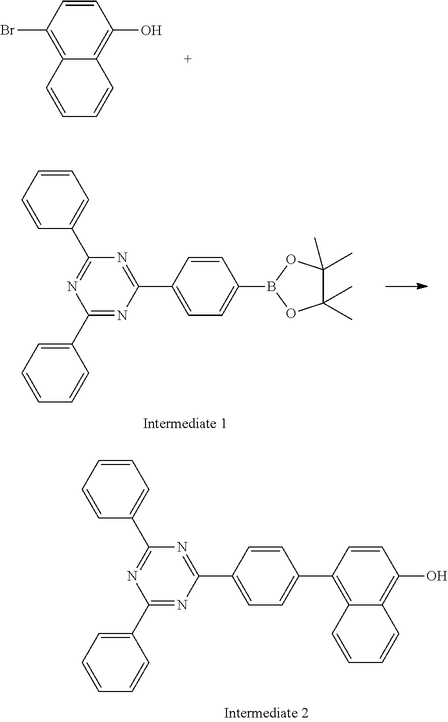 [SMILES: Oc1ccc(Br)c2ccccc12 ; CC1(C)OB(c2ccc(-c3nc(-c4ccccc4)nc(-c4ccccc4)n3)cc2)OC1(C)C ; Oc1ccc(-c2ccc(-c3nc(-c4ccccc4)nc(-c4ccccc4)n3)cc2)c2ccccc12]

4-bromonaphthalene-1-ol (17.7 g, 79 mmol) was mixed with the Intermediate 1 (23 g, 52.8 mmol) in dimethoxyethane (250 mL) and tetrakis(triphenylphosphine)palladium (0) (3,05 g, 2.64 mmol) in a 1000 mL of three-necked round flask replaced by nitrogen-flashing, and subsequently, 2 M of aqueous solution of sodium carbonate (79 mL, 158 mmol) was added thereto. The reaction mixture was heated at reflux for 6 hours. The reactant was cooled to room temperature, and then the solvents were removed under reduced pressure. The crude residue was suspended in 1 M of aqueous solution of HCl/methanol (1: 1, 500 mL), and then it was stirred at room temperature for an hour. The precipitate was collected by filtering, it was washed with water and methanol, and then it was dried. The obtained crude product was further purified by recrystallizing using xylene. 13.5 g (56.6%) of the obtained pale brown solid was used without further purification. Intermediate 2 was identified by using ESI-MS (electrospray ionization mass spectrometry). The molecular weight (M) calculated from the formula C₃₁ H₂₁N₃O of the Intermediate 2 is 451, and a mass measurement value (M+1) obtained using the ESI-MS was 452.

### (3) Synthesis of Intermediate 3

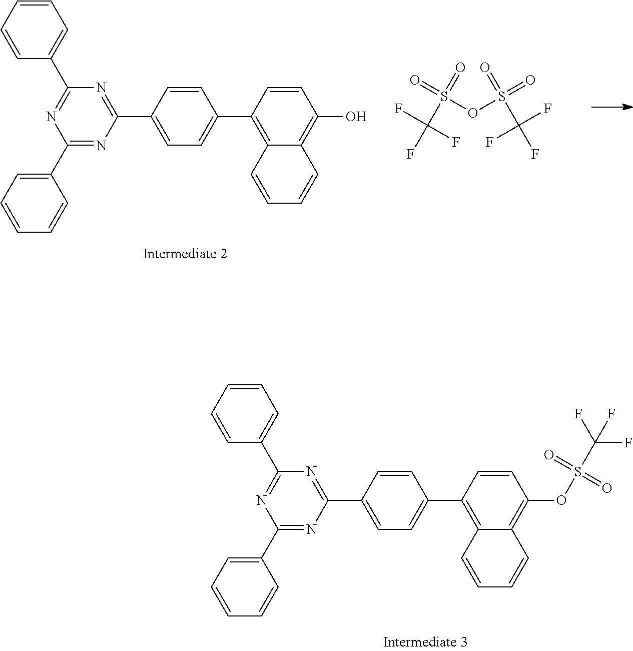 [SMILES: Oc1ccc(-c2ccc(-c3nc(-c4ccccc4)nc(-c4ccccc4)n3)cc2)c2ccccc12 ; O=S(=O)(OS(=O)(=O)C(F)(F)F)C(F)(F)F ; O=S(=O)(Oc1ccc(-c2ccc(-c3nc(-c4ccccc4)nc(-c4ccccc4)n3)cc2)c2ccccc12)C(F)(F)F]

Intermediate 2 (8.3 g, 18.38 mmol) was suspended in dichloromethane, and it was cooled in ice bath. Thereafter, 2,6-lutidine (4.3 ml, 36.8 mmol) was added thereto, and subsequently, trifluoromethanesulfonic acid anhydride (4.63 ml, 27.6 mmol) was added thereto. After an hour, the reaction was finished. The reaction mixture was washed with aqueous saturated sodium hydrogen carbonate, water, and brine, and then it was dried with anhydrous magnesium sulfate. The crude product was purified by silica chromatography using 20 to 40% of dichloromethane in heptane as eluent. 6.1 g (57%) of Intermediate 3 was isolated as pale brown oil, and then it was used without further purification. Intermediate 3 was identified by using ESI-MS (electrospray ionization mass spectrometry). The molecular weight (M) calculated from the formula C₃₂H₂₀F₃N₃O₃S of the Intermediate 3 is 583, and a mass measurement value (M+1) obtained using the ESI-MS was 584.

### (4) Synthesis of Compound C9

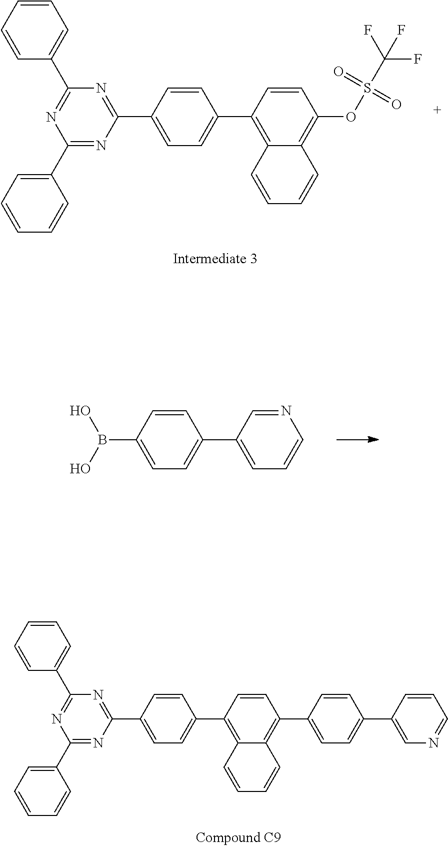 [SMILES: O=S(=O)(Oc1ccc(-c2ccc(-c3nc(-c4ccccc4)nc(-c4ccccc4)n3)cc2)c2ccccc12)C(F)(F)F ; OB(O)c1ccc(-c2cccnc2)cc1 ; c1ccc(-c2nc(-c3ccccc3)nc(-c3ccc(-c4ccc(-c5ccc(-c6cccnc6)cc5)c5ccccc45)cc3)n2)cc1]

Intermediate 3 (4 g, 6.8 mmol) was mixed with (4-(pyridine-3-yl)phenyl)boronic acid (1.2 g, 6.2 mmol), tetrakis(triphenylphosphine)palladium (0) (0.16 g, 0.14 mmol), and K₂CO₃ (1.9 g, 13.7 mmol) in a 250 mL of three-necked round flask replaced by nitrogen-flashing. Dioxane (30 mL) and water (7.5 mL) were added to the reaction mixture, and then it was heated overnight by using oil bath having temperature of 90° C. Subsequently, the reactant was cooled to room temperature, and then the solvents were removed under reduced pressure. Thereafter, the crude residue was suspended in methanol/water (1: 1, 100 mL), and then the mixture was stirred at room temperature for an hour. The precipitate was collected by filtering. Subsequently, the crude product was recrystallized using xylene, and then it was further purified using Train Sublimation method. The obtained Compound C9 (46% yield, white solid) was identified by using ESI-MS (electrospray ionization mass spectrometry), maximum ultraviolet absorption wavelength (UV(PhMe)λonset) in toluene, and maximum fluorescence wavelength (FL(PhMe, λex=330 nm)λmax) in toluene. The molecular weight (M) calculated from the formula C₄₂H₂₈N₄ of the Compound C9 is 588, and a mass measurement value (M+1) obtained using the ESI-MS was 589. Further, UV(PhMe)λonset is 384 nm, and FL(PhMe,λex=330 nm)λmax is 425 nm.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. An organic electroluminescence device comprising a cathode,
an anode,
an emitting layer arranged between the cathode and the anode, and an electron-transporting region arranged between the emitting layer and the cathode,
wherein the electron-transporting region comprises one or more compounds selected from the group consisting of compounds represented by the following formulas (1) to (4) and a rare earth element, and
the electron-transporting region substantially does not comprise an alkali metal, a compound containing an alkali metal, a metal belonging to Group 13 of the Periodic Table of the Elements, and a compound having a metal belonging to Group 13 of the Periodic Table of the Elements: 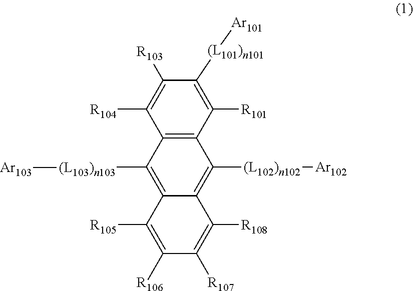
wherein in the formula (1),
R₁₀₁ and R₁₀₃ to R₁₀₈ are independently a hydrogen atom, or a substituent R;
L₁₀₁ is
a single bond,
a substituted or unsubstituted arylene group having 6 to 50 ring carbon atoms, or
a substituted or unsubstituted divalent heterocyclic group having 5 to 50 ring atoms;
L₁₀₂ and L₁₀₃ are independently
a single bond, or
a substituted or unsubstituted arylene group having 6 to 50 ring carbon atoms;
Ar₁₀₁ is
a substituted or unsubstituted monovalent group including a nitrogen-containing six-membered ring, or
a substituted or unsubstituted benzimidazolyl group;
Ar₁₀₂ and Ar₁₀₃ are independently a substituted or unsubstituted aryl group having 6 to 50 ring carbon atoms;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
n101 is an integer of 1 to 3; when n101 is 2 or more, two or more L₁₀₁'s may be the same as or different from each other;
n102 is an integer of 1 to 3; when n102 is 2 or more, two or more L₁₀₂'s may be the same as or different from each other;
n103 is an integer of 1 to 3; when n103 is 2 or more, two or more L₁₀₃'s may be the same as or different from each other: 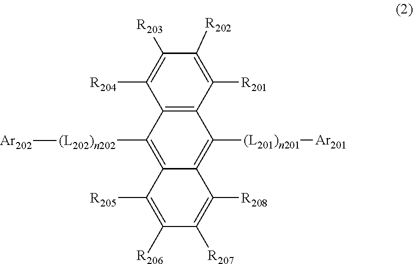
wherein in the formula (2),
R₂₀₁ to R₂₀₈ are independently a hydrogen atom, or a substituent R;
L₂₀₁ and L₂₀₂ are independently
a single bond, or
a substituted or unsubstituted arylene group having 6 to 50 ring carbon atoms;
Ar₂₀₁ and Ar₂₀₂ are independently a substituted or unsubstituted monovalent heterocyclic group having 5 to 50 ring atoms;
n201 is an integer of 1 to 3; when n201 is 2 or more, two or more L₂₀₁'s may be the same as or different from each other;
n202 is an integer of 1 to 3; when n202 is 2 or more, two or more L₂₀₂'s may be the same as or different from each other;
the substituent R is the same as defined in the formula (1): 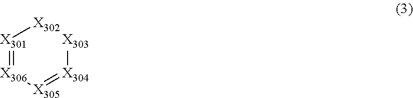
wherein in the formula (3),
X₃₀₁ to X₃₀₆ are independently N, CR₃₀₁ or CR₃₀₂; provided that at least two of X₃₀₁ to X₃₀₆ are N, at least one thereof is CR₃₀₁, and at least two thereof are CR₃₀₂; when two or more R₃₀₁'s are present, the two or more R₃₀₁'s may be the same as or different from each other;
two or more R₃₀₂'s may be the same as or different from each other;
R₃₀₁ is a monovalent group represented by the following formula (3A):
(Ar₃₀₁)_{m3A}-(L₃₀₁)_{n3A}-* (3A)
wherein in the formula (3A),
L₃₀₁ is
a single bond,
a substituted or unsubstituted aromatic hydrocarbon ring group having 6 to 50 ring carbon atoms, or
a substituted or unsubstituted heterocyclic group having 5 to 50 ring atoms;
Ar₃₀₁ is
a substituted or unsubstituted dibenzofuranyl group,
a substituted or unsubstituted dibenzothiophenyl group,
a substituted or unsubstituted carbazolyl group,
a substituted or unsubstituted phenanthryl group,
a substituted or unsubstituted monovalent group including a nitrogen-containing five-membered ring, or
a monovalent group represented by the following formula (3B): 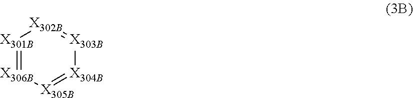
wherein in the formula (3B),
X_{301B} to X_{306B} are independently N, CR_{301B} or CR_{302B}; provided that one of X_{301B} to X_{306B} is CR_{301B}, and at least one thereof is N;
R_{301B} is a single bond bonding with L₃₀₁;
when two or more R_{302B}'s are present, the adjacent two of R_{302B}'s form a substituted or unsubstituted, saturated or unsaturated fused ring by bonding with each other, or do not form the substituted or unsubstituted, saturated or unsaturated fused ring;
R_{302B} which do not form the substituted or unsubstituted, saturated or unsaturated fused ring is a hydrogen atom, or a substituent R;
when two or more R_{302B}'s are present, the two or more R_{302B}'s may be the same as or different from each other;
m3A is an integer of 1 to 5; when m3A is 2 or more, two or more Ar₃₀₁'s may be the same as or different from each other;
n3A is an integer of 1 to 3; when n3A is 2 or more, two or more L₃₀₁'s may be the same as or different from each other; provided that when all of L₃₀₁'s are a single bond, m3A is 1;
R₃₀₂ is a hydrogen atom, or a substituent R;
the substituent R is the same as defined in the formula (1): 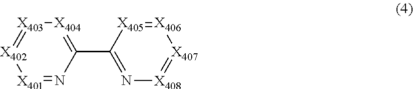
wherein in the formula (4),
X₄₀₁ to X₄₀₅ are independently N, CR₄₀₁ or CR₄₀₂; provided that at least one of X₄₀₁ to X₄₀₈ is CR₄₀₁; when two or more R₄₀₁'s are present, the two or more R₄₀₁'s may be the same as or different from each other; when two or more R₄₀₂'s are present, the two or more R₄₀₂'s may be the same as or different from each other;
when both of X₄₀₄ and X₄₀₅ are CR₄₀₂, the two R₄₀₂'s form a substituted or unsubstituted, saturated or unsaturated fused ring by bonding with each other, or do not form the substituted or unsubstituted, saturated or unsaturated fused ring;
R₄₀₁ is a monovalent group represented by the following formula (4A):
(Ar₄₀₁)_{m4A}—(L₄₀₁)_{n4A}—* (4A)
wherein in the formula (4A),
L₄₀₁ is
a single bond,
a substituted or unsubstituted aromatic hydrocarbon ring group having 6 to 50 ring carbon atoms, or
a substituted or unsubstituted heterocyclic group having 5 to 50 ring atoms;
Ar₄₀₁ is
a hydrogen atom,
a substituted or unsubstituted aryl group having 6 to 50 ring carbon atoms, or
a substituted or unsubstituted monovalent heterocyclic group having 5 to 50 ring atoms;
m4A is an integer of 1 to 5; when m4A is 2 or more, two or more Ar₄₀₁'s may be the same as or different from each other;
n4A is an integer of 1 to 3; when n4A is 2 or more, two or more L₄₀₁'s may be the same as or different from each other; provided that when all of L₄₀₁'s are a single bond, m4A is 1;
R₄₀₂ which do not form the substituted or unsubstituted, saturated or unsaturated fused ring is a hydrogen atom, or a substituent R; and
the substituent R is the same as defined in the formula (1).

2. The organic electroluminescence device according to claim 1,
wherein the compound represented by the formula (1) is a compound represented by the following formula (1-1): 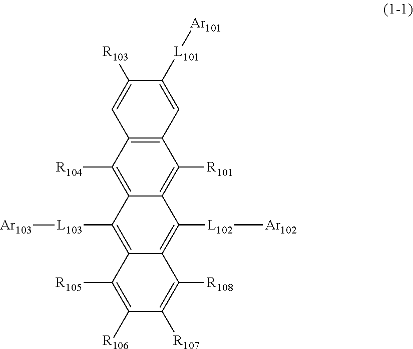
wherein in the formula (1-1),
R₁₀₁ and R₁₀₃ to Rios, L₁₀₁ to L₁₀₃, and Ar₁₀₁ to Ar₁₀₃ are the same as defined in the formula (1).

3-4. (canceled)

5. The organic electroluminescence device according to claim 1, wherein the compound represented by the formula (1) is a compound represented by the following formula (1-11): 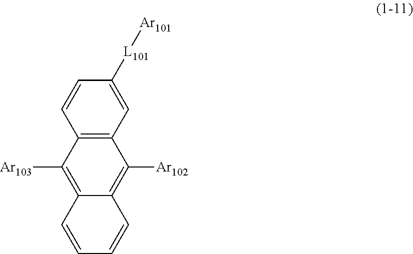
wherein in the formula (1-11),
L₁₀₁, and Ar₁₀₁ to Ar₁₀₃ are the same as defined in the formula (1).

6. The organic electroluminescence device according to claim 1, wherein the compound represented by the formula (2) is a compound represented by the following formula (2-1): 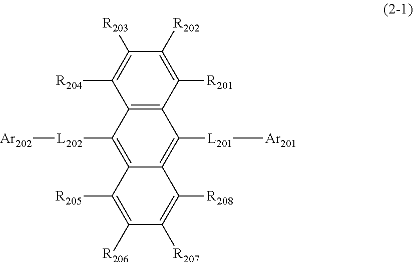
wherein in the formula (2-1),
R₂₀₁ to R₂₀₈, L₂₀₁, L₂₀₂, Ar₂₀₁, and Ar₂₀₂ are the same as defined in the formula (2).

7. The organic electroluminescence device according to claim 1, wherein Ar₂₀₁ in the formula (2) is a substituted or unsubstituted benzimidazolyl group.

8. (canceled)

9. The organic electroluminescence device according to claim 1, wherein the compound represented by the formula (2) is a compound represented by the following formula (2-11): 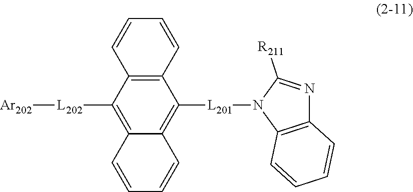
wherein in the formula (2-11),
L₂₀₁, L₂₀₂, and Ar₂₀₂ are the same as defined in the formula (2);
R₂₁₁ is a hydrogen atom, or a substituent R; and
the substituent R is the same as defined in the formula (1).

10. (canceled)

11. The organic electroluminescence device according to claim 1, wherein the compound represented by the formula (3) is a compound represented by the following formula (3-1): 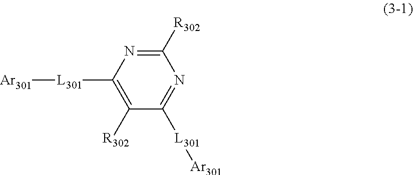
wherein in the formula (3-1),
R₃₀₂, L₃₀₁, and Ar₃₀₁ are the same as defined in the formula (3).

12. The organic electroluminescence device according to claim 1, wherein the compound represented by the formula (3) is a compound represented by the following formula (3-11): 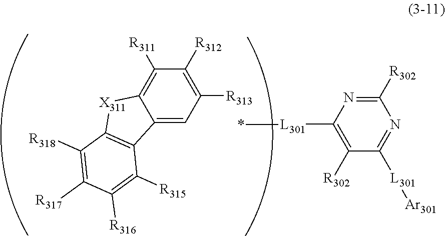
wherein in the formula (3-11),
R₃₀₂, L₃₀₁, and Ar₃₀₁ are the same as defined in the formula (3);
X₃₁₁ is O, S or NR₃₁₉;
one of R₃₁₁ to R₃₁₉ is bonded with L₃₀₁ via a single bond;
R₃₁₁ to R₃₁₉ which are not bonded with L₃₀₁ are independently a hydrogen atom, or a substituent R; and
the substituent R is the same as defined in the formula (1).

13. (canceled)

14. The organic electroluminescence device according to claim 1, wherein the compound represented by the formula (3) is a compound represented by the following formula (3-2): 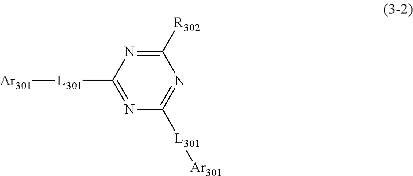
wherein in the formula (3-2),
R₃₀₂, L₃₀₁, and Ar₃₀₁ are the same as defined in the formula (3).

15. The organic electroluminescence device according to claim 1, wherein the compound represented by the formula (3) is a compound represented by the following formula (3-21): 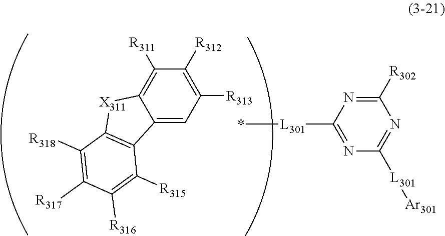
wherein in the formula (3-21),
R₃₀₂, L₃₀₁, and Ar₃₀₁ are the same as defined in the formula (3);
X₃₁₁ is O, S or NR₃₁₉;
one of R₃₁₁ to R₃₁₉ is bonded with L₃₀₁ via a single bond;
R₃₁₁ to R₃₁₉ which are not bonded with L₃₀₁ are independently a hydrogen atom, or a substituent R; and
the substituent R is the same as defined in the formula (1).

16. (canceled)

17. The organic electroluminescence device according to claim 1, wherein the compound represented by the formula (3) is a compound represented by the following formula (3-3): 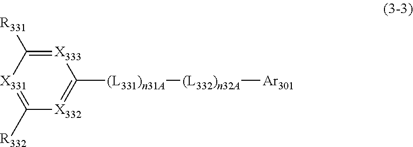
wherein in the formula (3-3), Ar₃₀₁ is the same as defined in the formula (3);
X₃₃₁ to X₃₃₃ are independently N or CR₃₃₃; provided that at least two of X₃₃₁ to X₃₃₃ are N;
R₃₃₁ and R₃₃₂ are independently
a substituted or unsubstituted alkyl group having 1 to 25 carbon atoms,
a substituted or unsubstituted cycloalkyl group having 3 to 18 ring carbon atoms,
a cyano group,
a substituted or unsubstituted aryl group having 6 to 30 ring carbon atoms, or
a substituted or unsubstituted monovalent heterocyclic group having 5 to 30 ring atoms;
R₃₃₃ is
a hydrogen atom,
a substituted or unsubstituted alkyl group having 1 to 25 carbon atoms,
a substituted or unsubstituted cycloalkyl group having 3 to 18 ring carbon atoms,
a cyano group,
a substituted or unsubstituted aryl group having 6 to 30 ring carbon atoms, or
a substituted or unsubstituted monovalent heterocyclic group having 5 to 30 ring atoms;
L₃₃₁ is
a substituted or unsubstituted aromatic hydrocarbon ring group having 6 to 30 ring carbon atoms, or
a substituted or unsubstituted heterocyclic group having 5 to 30 ring atoms;
L₃₃₂ is
a substituted or unsubstituted aromatic hydrocarbon ring group having 6 to 30 ring carbon atoms, or
a substituted or unsubstituted heterocyclic group containing at least one nitrogen atom and having 5 to 30 ring atoms;
n31A is an integer of 0 to 2; when n31A is 2, two L₃₃₁'s may be the same as or different from each other;
n32A is an integer of 0 to 2; when n32A is 2, two L₃₃₂'s may be the same as or different from each other; and
provided that the sum of n31A and n32A is two or more.

18. The organic electroluminescence device according to claim 17, wherein the compound represented by the formula (3-3) is a compound represented by the following formula (3-31): 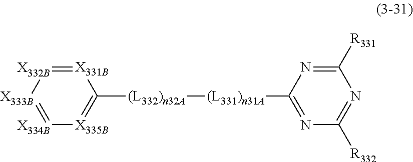
wherein in the formula (3-31),
R₃₃₁, R₃₃₂, L₃₃₁, L₃₃₂, n31A, and n32A are the same as defined in the formula (3-3);
X_{331B} to X_{335B} are independently N or CR_{331B}; provided that at least one of X₃₃₁B to X_{335B} are N;
when two or more R_{331B}'s are present, the adjacent two of R_{331B}'s form a substituted or unsubstituted, saturated or unsaturated fused ring by bonding with each other, or do not form the substituted or unsubstituted, saturated or unsaturated fused ring;
R_{331B} which do not form the substituted or unsubstituted, saturated or unsaturated fused ring is a hydrogen atom, or a substituent R;
when two or more R_{331B}'s are present, the two or more R_{331B}'s may be the same as or different from each other; and
the substituent R is the same as defined in the formula (1).

19. The organic electroluminescence device according to claim 1, wherein the compound represented by the formula (4) is a compound represented by the following formula (4-1) or (4-2): 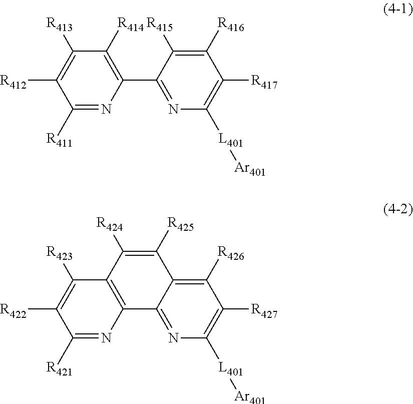
wherein in the formulas (4-1) and (4-2),
L₄₀₁, and Ar₄₀₁ are the same as defined in the formula (4);
R₄₁₁ to R₄₁₇, and R₄₂₁ to R₄₂₇ are independently a hydrogen atom, or a substituent R; and
the substituent R is the same as defined in the formula (1).

20. The organic electroluminescence device according to claim 1, wherein the monovalent group represented by the formula (4A) is a monovalent group represented by the following formula (4A-1): 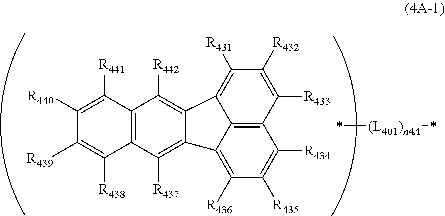
wherein in the formula (4A-1), L₄₀₁ and n4A are the same as defined in the formula (4A);
one of R₄₃₁ to R₄₄₂ is bonded with L₄₀₁ via a single bond;
R₄₃₁ to R₄₄₂ which are not bonded with L₄₀₁ are independently a hydrogen atom, or a substituent R; and
the substituent R is the same as defined in the formula (1).

21. The organic electroluminescence device according to claim 1, wherein the compound represented by the formula (4) is a compound represented by the following formula (4-11) or (4-12): 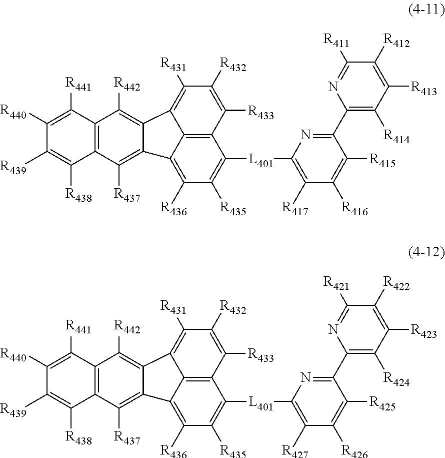
wherein in the formulas (4-11) and (4-12),
L₄₀₁ is the same as defined in the formula (4);
R₄₁₁ to R₄₁₇, R₄₂₁ to R₄₂₇, R₄₃₁ to R₄₃₃, and R₄₃₅ to R₄₄₂ are independently a hydrogen atom, or a substituent R; and
the substituent R is the same as defined in the formula (1).

22. The organic electroluminescence device according to claim 1, wherein the monovalent group represented by the formula (4A) is a monovalent group represented by the following formula (4A-2): 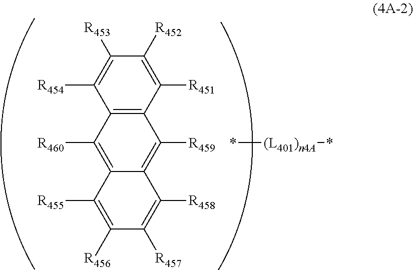
wherein in the formula (4A-2), L₄₀₁ and n4A are the same as defined in the formula (4A);
one of R₄₅₁ to R₄₆₀ is bonded with L₄₀₁ via a single bond;
R₄₅₁ to R₄₆₀ which are not bonded with L₄₀₁ are independently a hydrogen atom, or a substituent R; and
the substituent R is the same as defined in the formula (1).

23. (canceled)

24. The organic electroluminescence device according to claim 1, wherein the compound represented by the formula 4 is a compound represented by the following formula (4-22-1) or (4-22-2): 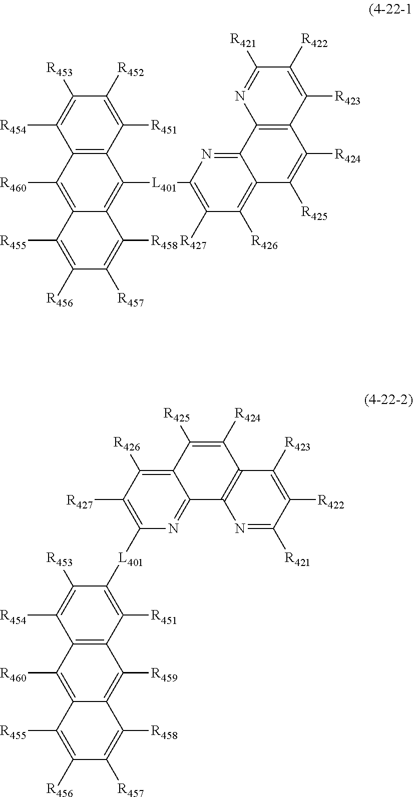
wherein in the formulas (4-22-1) and (4-22-2),
L₄₀₁ is the same as defined in the formula (4);
R₄₂₁ to R₄₂₇, and R₄₅₁ to R₄₆₀ which are not bonded with L₄₀₁ are independently a hydrogen atom, or a substituent R; and
the substituent R is the same as defined in the formula (1).

25. The organic electroluminescence device according to claim 1, wherein the electron-transporting region comprises a first layer and a second layer in this order from the emitting layer side,
the first layer comprises one or more compounds selected from the group consisting of compounds represented by the formulas (1) to (4), and
the second layer comprises the rare earth element.

26. The organic electroluminescence device according to claim 25, wherein the electron-transporting region comprises a third layer between the emitting layer and the first layer, and
the electron-transporting region does not comprise other layers between the emitting layer and the third layer.

27. The organic electroluminescence device according to claim 26, wherein the electron-transporting region comprises a fourth layer between the third layer and the first layer.

28. (canceled)
